(19) United States
(12) Patent Application Publication
(10) Pub. No.: US 2025/0195513 A1
(43) Pub. Date: Jun. 19, 2025

(54) DEGRADATION OF IRAK4 BY CONJUGATION OF IRAK4 INHIBITORS WITH E3 LIGASE LIGAND AND METHODS OF USE

(71) Applicant: BeiGene, Ltd., Camana Bay (KY)

(72) Inventors: Huaqing LIU, Beijing (CN); Dongqing SUN, Beijing (CN); Zhiwei WANG, Beijing (CN)

(73) Assignee: BeiGene, Ltd., Camana Bay (KY)

(21) Appl. No.: 18/972,490

(22) Filed: Dec. 6, 2024

Related U.S. Application Data
(63) Continuation of application No. PCT/CN2023/099146, filed on Jun. 8, 2023.

(30) Foreign Application Priority Data
Jun. 9, 2022 (WO) PCT/CN2022/097833
Oct. 28, 2022 (WO) PCT/CN2022/128219
May 11, 2023 (WO) PCT/CN2023/093639

(51) Int. Cl. A61K 31/5025; C07D 487/04

(52) U.S. Cl. CPC A61K 31/5025; C07D 487/04

(57) ABSTRACT

Disclosed herein are novel bifunctional compounds formed by conjugating IRAK4 inhibitor moieties with E3 ligase ligand moieties, which function to recruit targeted proteins to E3 ubiquitin ligase for degradation, and methods of preparation and uses thereof.

### FIELD OF THE INVENTION

Disclosed herein are novel bifunctional compounds formed by conjugating IRAK4 inhibitor moieties with E3 ligase ligand moieties, which function to recruit targeted proteins to E3 ubiquitin ligase for degradation, and methods of preparation and uses thereof.

### BACKGROUND OF THE INVENTION

Proteolysis targeting chimera (PROTAC) consists of two covalently linked protein-binding molecules: one capable of engaging an E3 ubiquitin ligase, and the other that binds to the protein of interest (POI) a target meant for degradation (Sakamoto K M et al., Proc. Natl. Acad. Sci. 2001, 98: 8554-9; Sakamoto K. M. et al., Methods Enzymol. 2005; 399:833-847). Rather than inhibiting the target protein's enzymatic activity, recruitment of the E3 ligase to the specific unwanted proteins results in ubiquitination and subsequent degradation of the target protein by the proteasome. The whole process of ubiquitination and proteasomal degradation is known as the ubiquitin-proteasome pathway (UPP) (Ardley H. et al., Essays Biochem. 2005, 41, 15-30; Komander D. et al., Biochem. 2012, 81, 203-229; Grice G. L. et al., Cell Rep. 2015, 12, 545-553; Swatek K. N. et al., Cell Res. 2016, 26, 399-422; Lydia M. et al., ACS Infect. Dis. 2019, 5, 12, 2105-2117). Proteasomes are protein complexes which degrade unneeded, misfolded or abnormal proteins into small peptides and amino acids to maintain health and productivity of the cells. Ubiquitin ligases, also called an E3 ubiquitin ligase, directly catalyze the transfer of ubiquitin from the E2 to the target protein for degradation. Although the human genome encodes over 600 putative E3 ligases, only a limited number of E3 ubiquitin ligases have been widely applied by small molecule PROTAC technology: cereblon (CRBN), Von Hippel-Lindau (VHL), mouse double minute 2 homologue (MDM2) and cellular inhibitor of apoptosis protein (cIAP) (Philipp O. et al., Chem. Biol. 2017, 12, 2570-2578), recombinant Human Ring Finger Protein 114 (RNF114) (Spradlin, J. N. et al. Nat. Chem. Biol. 2019, 15, 747-755) and DDB1 And CUL4 Associated Factor 16 (DCAF16) (Zhang, X. et al. Nat. Chem. Biol. 2019, 15, 737-746). For example, cereblon (CRBN) forms an E3 ubiquitin ligase complex with damaged DNA binding protein 1 (DDB1) and Cullin-4A (CUL4A) to ubiquitinate a number of other proteins followed by the degradation via proteasomes. (Yi-An Chen, et al., Scientific Reports 2015, 5, 1-13). Immunomodulatory drugs (IMiDs), including thalidomide, lenalidomide, and pomalidomide, function as monovalent promoters of PPIs by binding to the cereblon (CRBN) subunit of the CRL4ACRBN E3 ligase complex and recruiting neosubstrate proteins. (Matyskiela, M. E. et al., Nat Chem Biol 2018, 14, 981-987.) As a consequence, the ability of thalidomide, and its derivatives, to recruit CRBN has been widely applied in proteolysis-targeting chimeras (PROTACs) related studies (Christopher T. et al. ACS Chem. Biol. 2019, 14, 342-347; Honorine L. et al, ACS Cent. Sci. 2016, 2, 927-934). PROTACs have great potential to eliminate protein targets that are “undruggable” by traditional inhibitors or are non-enzymatic proteins. (Chu T T. et al., Cell Chem Biol. 2016; 23:453-461. Qin C. et al., J Med Chem 2018; 61: 6685-6704. Winter G E. et al., Science 2015; 348:1376-1381.) In the recent years, PROTACs as useful modulators that promote selective degradations of a wide range of target proteins have been reported in antitumor studies. (Lu J. et al., Chem Biol. 2015; 22(6):755-763; Ottis P. et al., Chem Biol. 2017; 12(4):892-898; Crews C. M. et al., J Med Chem. 2018; 61(2):403-404; Neklesa T. K. et al., Pharmacol Ther. 2017, 174:138-144; Cermakova K. et al., Molecules, 2018.23(8); An S. et al., EBioMedicine, 2018; Lebraud H. et al., Essays Biochem. 2017; 61(5): 517-527; Sun Y. H. et al., Cell Res. 2018; 28:779-81; Toure M. et al., Angew Chem Int Ed Engl. 2016; 55(6):1966-1973; Yonghui Sun et al., Leukemia, volume 33, pages 2105-2110(2019); Shaodong Liu et al., Medicinal Chemistry Research, volume 29, pages 802-808 (2020); and has been disclosed or discussed in patent publications, e.g., US20160045607, US20170008904, US20180050021, US20180072711, WO2002020740, WO2014108452, WO2016146985, WO2016149668, WO2016197032, WO2016197114, WO2017011590, WO2017030814, WO2017079267, WO2017182418, WO2017197036, WO2017197046, WO2017197051, WO2017197056, WO2017201449, and WO2018071606.

Interluekin-1 receptor associated kinases (IRAK1, IRAK2, IRAK3 and IRAK4) are serine-threonine kinases that regulate innate immune and inflammatory responses. In addition to performing normal kinase functions, IRAK4 has protein scaffolding functions and regulates downstream signaling via the myddosome protein complex. Activations of TLRs or IL-1Rs initiate receptor dimerization and recruit the myeloid differentiation primary response 88 (MyD88) adaptor protein through their intracellular domains. MyD88 further recruits IRAK4 and IRAK1/2 to form a multiprotein complex, the myddosome.

Interaction of MyD88 and IRAK4 firstly recruits pre-formed IRAK-1. Then IRAK4 phosphorylates and activates IRAK-1. Fully activated IRAK-1 recruits TRAF6-TAK1-TAB-1/2 complex. Activated TAK-1 phosphorylates downstream signaling cascades including the IκB kinase (IKK)-nuclear factor-κB(NF-κB) and MAPKs such as c-Jun N-terminal kinase (JNK) and p38, leading to production of proinflammatory cytokines (including IL-1β, IL-6, IL-8, IL-12, TNF). These signaling pathways play crucial roles in both innate immune response and adaptive immunity (Elizabeth L. et al., Eur. J. Immunol. 2008, 38, 3, 870-876).

The pro-inflammatory role of IRAK4 has been demonstrated with studies of IRAK4 kinase dead, knock-in mice (W. Michael S., et al., ACS Med. Chem. Lett. 2015, 6, 942-947). Mice possessing this genotype are resistant to joint inflammation in several rodent arthritis models. (Koziczak-Holbro et al, 2009, Arthritis & rheumatism). IRAK4 deficiency ameliorates disease activity in multiple RA animal models. Additionally, a small IRAK4 deficient human population has been identified. Reduced pro-inflammatory cytokine expressions from macrophage, reduced fibroblast like synoviocytes inflammation and migration were observed. Cells from these patients have impaired responses to ILR/TLR receptor stimulation. No severe viral, fungal, or parasitic infections were observed in those adult patients (Gosu, V. et al., Sci Rep 4, 2014, 5748; Shichijo K. et al., Pediatrics International, 2015). These data in rodents and humans implies that an IRAK4 inhibitor could modulate the production of key inflammatory cytokines and cytokine induced pathologies.

However, there are reports that pharmacological inhibition of IRAK4 did not result in IL-6 and TNF-α inhibition despite IRAK4 phosphorylation levels being reduced in IL-1β stimulated human dermal fibroblasts, which suggests that the kinase activity may not be the sole factor in certain cell types (O'Neill, L. A. Immunol. Rev. 2008, 226, 10-8; Nunes J., et al. ACS Med. Chem. Lett. 2019, 10, 1081). Therefore, removing the IRAK4 scaffolding function in addition to the inhibition of kinase activities may offer a better therapeutic outcome. In this regard, IRAK4-targeting PROTACs may serve as a potential strategy to targeting both IRAK4 kinase activity and scaffolding function. IRAK4 PROTACs could ultimately lead to new therapeutic opportunities to treat autoimmune, inflammatory, and oncological diseases. Following patents have disclosed or discussed the IRAK4 PROTACs: WO2019133531, US20190192668, WO2019099926, WO2020113233, WO2020264499, WO2021158634, WO2020264490, WO2021119159, WO2021168197 and WO2021127278.

Recently some IRAK4-targeting PROTACs have been published (Nunes J., et al. ACS Med. Chem. Lett. 2019, 10, 1081; Zhang et al., Cell Chem. Bio. 2020, 27, 1; Robert B. K. et al. ACS Med. Chem. Lett. 2019, 10, 1251). Most of these molecules are based on published IRAK4 inhibitors as warheads. However, there were few data showed those IRAK4 PROTACs gave stronger inhibition of cytokine productions such as IL-6 and TNF-α than IRAK4 kinase inhibitors. In this following invention, well designed IRAK4 PROTACs have demonstrated superior reductions of proinflammatory cytokines in multiple cell lines than small molecular inhibitors.

The present application provides novel bifunctional compounds and compositions for the treatment of autoimmune, inflammatory, and oncological diseases.

### SUMMARY OF THE INVENTION

In one embodiment, disclosed herein is bifunctional compounds of Formula (I) that can selectively degrade IRAK4. The compounds described herein or salts thereof are useful in the treatment of a disease that can be affected by IRAK4 modulation. The present invention provides the use of the compounds described herein or a pharmaceutically acceptable salt thereof in the manufacture of a medicament for the treatment of a disease that can be affected by IRAK4 modulation. The present invention further provides a compound described herein or a pharmaceutically acceptable salt thereof, for use in the treatment of a disease that can be affected by IRAK4 modulation. The present application further provides a method of treating a proliferative disorder, comprising administering to a subject in need thereof a therapeutically effective amount of the compounds described herein or a pharmaceutically acceptable salt thereof. The embodiment comprises the following aspects:

Aspect 1. A compound of formula (X)

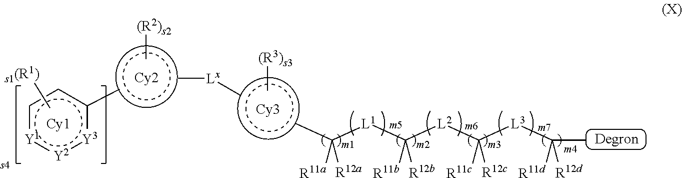

or a N-oxide thereof, or a pharmaceutically acceptable salt thereof, or a stereoisomer thereof, or a deuterated analog thereof, or a prodrug thereof,
wherein:
L^{x} is *^{Lx}—CONH—**^{Lx} or *^{Lx}—NHCO—**^{Lx}, where in *^{Lx} refers to the position attached to the Cy2 moiety, and **^{Lx} refers to the position attached to the Cy3 moiety;
Cy1 is a 6-membered partially or completely unsaturated ring; said ring is substituted with at least one substituent R¹;
Y¹, Y² and Y³ are each independently C or N;
s1 is 0, 1, 2, 3, 4 or 5;
Cy2 is a 5-, 6-, 7-, 8-, 9- or 10-membered partially or completely unsaturated ring (aromatic ring); said ring comprising 0-3 heteroatoms independently selected from nitrogen, oxygen or optionally oxidized sulfur; said ring is substituted with at least one substituent R²;
s2 is 0, 1 or 2;
s4 is 0 or 1;
Cy3 is a 5-, 6-, 7-, 8-, 9- or 10-membered aromatic ring; said ring comprising 0-3 heteroatoms independently selected from nitrogen, oxygen or optionally oxidized sulfur; said ring is substituted with at least one substituent R³;
s3 is 0, 1, 2, 3, 4 or 5;
R¹, R², and R³ are each independently hydrogen, halogen, —C₁-C₈alkyl, —C₃-C₈cycloalkyl, 3- to 8-membered heterocyclyl, C₆-C₁₂aryl, 5- to 12-membered heteroaryl, oxo(═O), —CN, —OR^{1a}, —C(O)R^{1a}—, —CO₂R^{1a}, —C(O)NR^{1a}R^{1b}, —NR^{1a}R^{1b}, —NR^{1a}COR^{1b} or —NR^{1a}CO₂R^{1b}; each of —C₁-C₈alkyl, C₃-C₈cycloalkyl, 3- to 8-membered heterocyclyl, C₆-C₁₂aryl or 5- to 12-membered heteroaryl is optionally substituted with at least one substituent R^{1c}; or
when adjacent, two R¹, two R² or two R³ together with the atoms to which they are attached, form a 3- to 12-membered ring, said ring comprising 0-3 heteroatoms independently selected from nitrogen, oxygen or sulfur; said ring is optionally substituted with at least one substituent R^{1c};
   R^{1a} and R^{1b} are each independently selected from hydrogen, —C₁-C₈alkyl, —C₁-C₈haloalkyl, —C₂-C₈alkenyl, —C₂-C₈alkynyl, C₁-C₈alkoxy-C₁-C₈alkyl-, C₃-C₈cycloalkyl, 3- to 8-membered heterocyclyl, C₆-C₁₂aryl or 5- to 12-membered heteroaryl; each of —C₁-C₈alkyl, —C₁-C₈haloalkyl, —C₂-C₈alkenyl, —C₂-C₈alkynyl, C₁-C₈alkoxy-C₁-C₈alkyl-, C₃-C₈cycloalkyl, 3- to 8-membered heterocyclyl, C₆-C₁₂aryl or 5- to 12-membered heteroaryl is optionally substituted with at least one substituent selected from halogen, —OH, —CN, oxo (═O), —C₁-C₈alkyl, —C₁-C₈alkyl-OH, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;
   R^{1c}, at each occurrence, is independently halogen, —OH, —CN, oxo (═O), —C₁-C₈alkyl, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;
R^{11a}, R^{11b}, R^{11c}, R^{11d}, R^{12a}, R^{12b}, R^{12c} and R^{12d} are each independently absent, oxo, hydrogen, halogen, —C₁₋₈alkyl, —C₂₋₈alkenyl, —C₂₋₈alkynyl, —C₁₋₈alkoxy or —C₃₋₈cycloalkyl; each of said —C₁₋₈alkyl, —C₂₋₈alkenyl, —C₂₋₈alkynyl, —C₁₋₈alkoxy or —C₃₋₈cycloalkyl is optionally substituted with at least one substituent selected from hydrogen, halogen, —C₁₋₈alkyl, —C₂₋₈alkenyl, —C₂₋₈alkynyl, —C₁₋₈alkoxy or —CN; or
   R^{11a} and R^{12a} together with the carbon atoms to which they are attached, form a 3- to 12-membered ring, said ring comprising 0-3 heteroatoms independently selected from nitrogen, oxygen or sulfur; said ring is optionally substituted with at least one substituent selected from hydrogen, halogen, —C₁₋₈alkyl, —C₂₋₈alkenyl, —C₂₋₈alkynyl, —C₁₋₈alkoxy or —CN; and/or
   R^{11b} and R^{12b} together with the carbon atoms to which they are attached, form a 3- to 12-membered ring, said ring comprising 0-3 heteroatoms independently selected from nitrogen, oxygen or sulfur; said ring is optionally substituted with at least one substituent selected from hydrogen, halogen, —C₁₋₈alkyl, —C₂₋₈alkenyl, —C₂₋₈alkynyl, —C₁₋₈alkoxy or —CN; and/or
   R^{11c} and R^{12c} together with the carbon atoms to which they are attached, form a 3- to 12-membered ring, said ring comprising 0-3 heteroatoms independently selected from nitrogen, oxygen or sulfur; said ring is optionally substituted with at least one substituent selected from hydrogen, halogen, —C₁₋₈alkyl, —C₂₋₈alkenyl, —C₂₋₈alkynyl, —C₁₋₈alkoxy or —CN; and/or
   R^{11d} and R^{12d} together with the carbon atoms to which they are attached, form a 3- to 12-membered ring, said ring comprising 0-3 heteroatoms independently selected from nitrogen, oxygen or sulfur; said ring is optionally substituted with at least one substituent selected from hydrogen, halogen, —C₁₋₈alkyl, —C₂₋₈alkenyl, —C₂₋₈alkynyl, —C₁₋₈alkoxy or —CN;
each of occurrence, L¹ is independently selected from —O—, —NR^{a}—, —C(O)NR^{a}—, —C(O)O—,

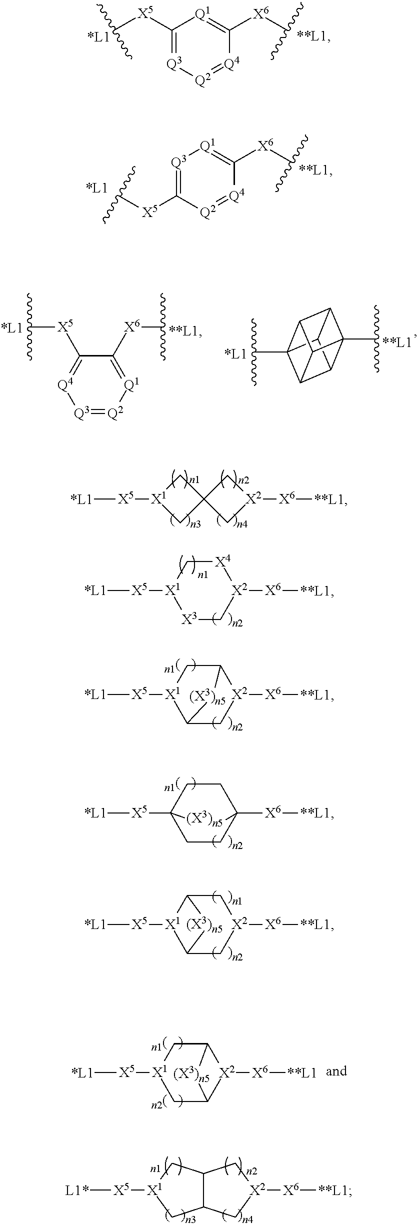

wherein each of said

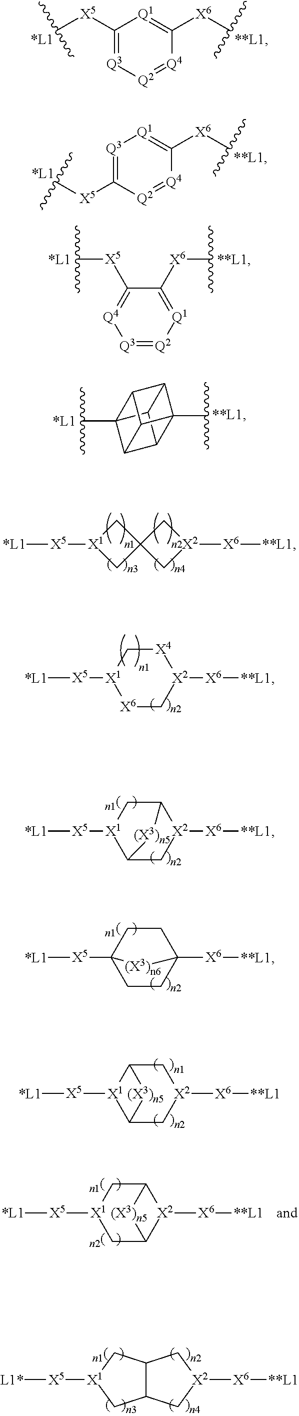

is optionally substituted with at least one R^{L1c};
wherein *^{L1} refers to the position attached to the

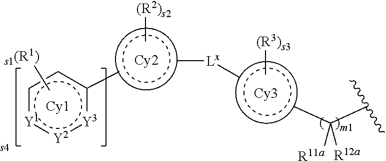

moiety, and **^{L1} refers to the position attached to the

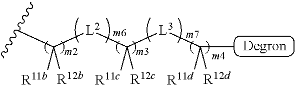

moiety;
each of occurrence, L² is independently selected from —O—, —NR^{a}—, —CONR^{a}—, —C(O)O—,

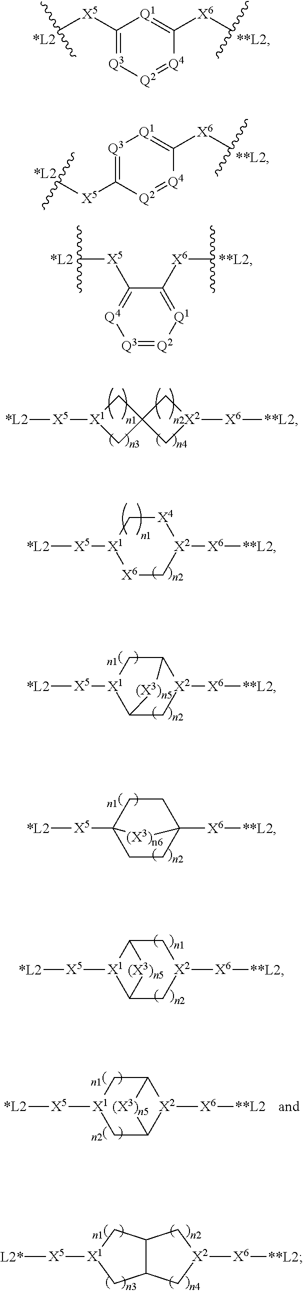

wherein each of said

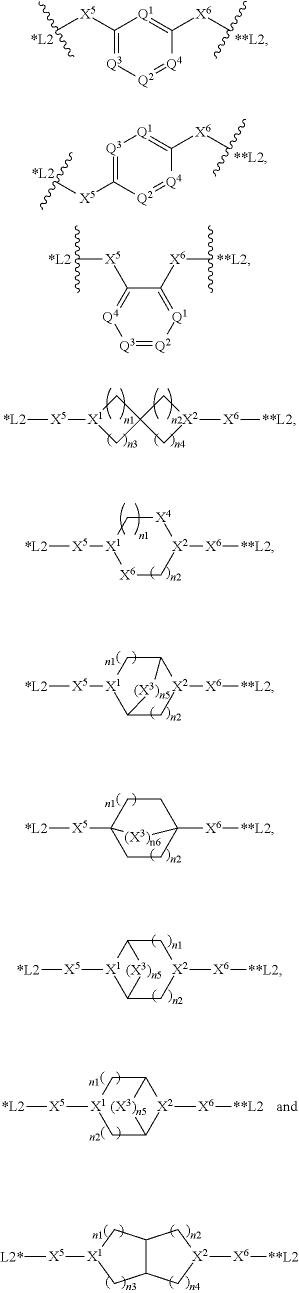

is optionally substituted with at least one R^{L2c};
wherein *^{L2} refers to the position attached to the

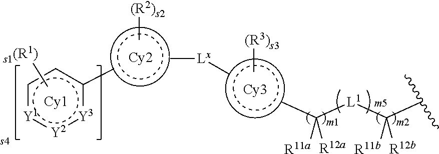

moiety, and **^{L2} refers to the position attached to the

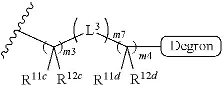

moiety;
each of occurrence, L³ is independently selected from —O—, —NR^{a}—, —CONR^{a}—, —C(O)O—,

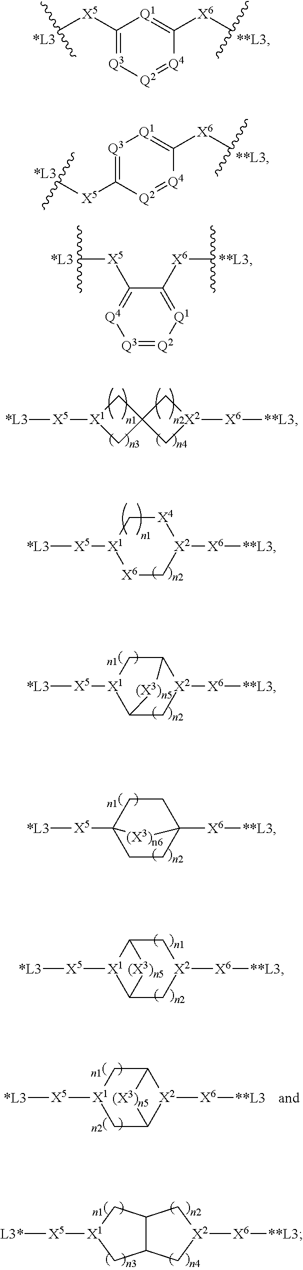

wherein each of said

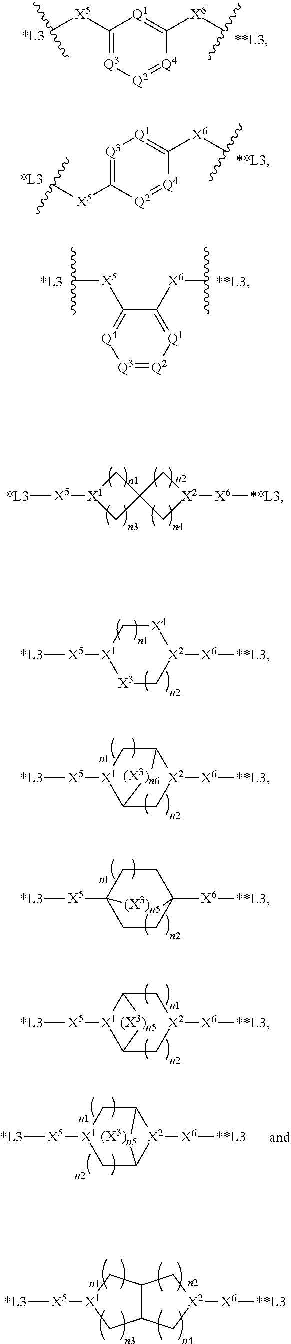

is optionally substituted with at least one R^{L3c};
wherein *^{L3} refers to the position attached to the

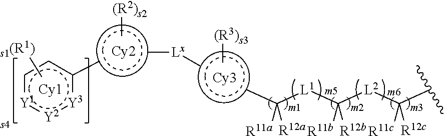

moiety, and **^{L3} refers to the position attached to the

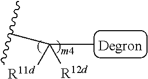

moiety;
each of said R^{L1c}, R^{L2c} and R^{L3c} are independently absent, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; each of said —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl and 5- to 12-membered heteroaryl is optionally substituted with at least one R^{Lca},
R^{Lca} is independently absent, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or
two R^{L1c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L2c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L3c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
[symbol] is selected from

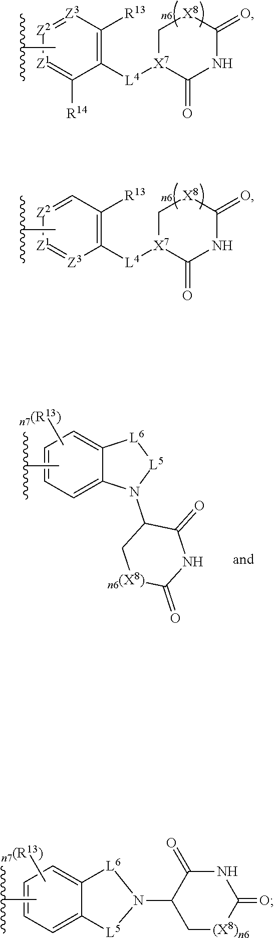

Z¹, Z² and Z³ are each independently N or CR^{z}, provided that Z¹, Z² and Z³ are not N at the same time;
R^{z}, at each occurrence, is independently selected from absent, hydrogen, halogen, —C₁₋₈alkyl, —NR^{Za}R^{Zb}, —OR^{Za}, —SR^{Za}, C₃-C₈cycloalkyl, 3- to 8-membered heterocyclyl or CN; each of —C₁₋₈alkyl, C₃-C₈cycloalkyl, 3- to 8-membered heterocyclyl is optionally substituted with at least one R^{Zc};
the

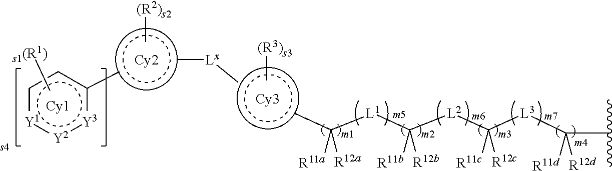

moiety is linked to the

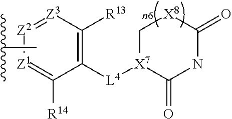

moiety via any one of Z¹, Z² or Z³ which is CR^{z} and R^{z} is absent;
R^{Za} and R^{Zb} are each independently selected from absent, hydrogen, —C₁-C₈alkyl, C₃-C₈cycloalkyl, 3- to 8-membered heterocyclyl, C₆-C₁₂aryl, or 5- to 12-membered heteroaryl, each of said —C₁₋₈alkyl, C₃-C₈cycloalkyl, 3- to 8-membered heterocyclyl, C₆-C₁₂aryl, or 5- to 12-membered heteroaryl is optionally substituted with at least one substituent R^{Zd};
R^{Zc} and R^{Zd} are each independently halogen, hydroxy, —C₁-C₈alkyl, —C₁₋₈alkoxy, C₃-C₈cycloalkyl, 3- to 8-membered heterocyclyl, C₆-C₁₂aryl, or 5- to 12-membered heteroaryl;
R¹³ and R¹⁴ are each independently selected from absent, hydrogen, halogen, —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}, —NR^{13a}CO₂R^{13b}, or —NR^{13a}SO₂R^{13b}; each of —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl or 5- to 12-membered heteroaryl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{13c}, —CONR^{13c}R^{13d}, —NR^{13c}R^{13d}, —NR^{13c}COR^{13d}, —NR^{13c}CO₂R^{13d}, or —NR^{13c}SO₂R^{13d};
at each occurrence, R^{13a}, R^{13b}, R^{13c} and R^{13d} are each independently absent, hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl, or 5- to 12-membered heteroaryl;
L⁴, L⁵ and L⁶ are each independently selected from a absent, single bond, —O—, —NR^{a}—, —(CR^{a}R^{b})ₙ₈—, —O(CR^{a}R^{b})ₙ₈—, —NR^{a}(CR^{a}R^{b})ₙ₈— or —C(O)—;
at each occurrence, X¹, X² and X⁷ are each independently selected from —CR^{a}, or N;
at each occurrence, X³, X⁴ and X⁸ are each independently selected from —NR^{a}—, —O—, —S— and —CR^{a}R^{b}—;
at each occurrence, X⁵ and X⁶ are each independently selected from absent, single bond, —C(O)—, —NR^{a}— and —O—;
Q¹, Q², Q³ and Q⁴ are each independently selected from CR^{a} or N;
at each occurrence, R^{a} and R^{b} are each independently selected from hydrogen, hydroxy, halogen, CN, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl, each of said —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl is optionally substituted with at least one substituent halogen, hydroxy, halogen, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; or
R^{a} and R^{b} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl, —C₂-C₈alkenyl, —C₂-C₈alkynyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl;
m1, m2, m3 and m4 are each independently 0, 1 or 2;
m5 and m7 are each independently 0 or 1;
m6 is 0, 1 or 2; when m6 is 2, two L2 are same or different;
n1, n2, n3, n4 and n5 are each independently 0, 1, 2 or 3;
n6, n7 and n8 are each independently 0, 1, 2, 3 or 4.

Aspect 2. A compound of formula (I)

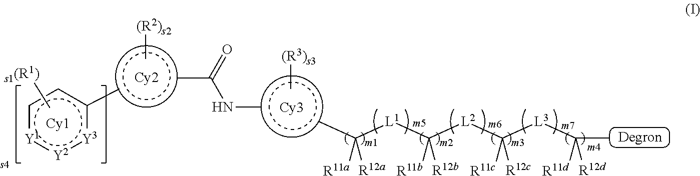

or a N-oxide thereof, or a pharmaceutically acceptable salt thereof, or a stereoisomer thereof, or a deuterated analog thereof, or a prodrug thereof,
wherein:
Cy1 is a 6-membered partially or completely unsaturated ring; said ring is substituted with at least one substituent R¹;
Y¹, Y² and Y³ are each independently C or N;
s1 is 0, 1, 2, 3, 4 or 5;
Cy2 is a 5-, 6-, 7-, 8-, 9- or 10-membered partially or completely unsaturated ring (aromatic ring); said ring comprising 0-3 heteroatoms independently selected from nitrogen, oxygen or optionally oxidized sulfur; said ring is substituted with at least one substituent R²;
s2 is 0, 1 or 2;
s4 is 0 or 1;
Cy3 is a 5-, 6-, 7-, 8-, 9- or 10-membered aromatic ring; said ring comprising 0-3 heteroatoms independently selected from nitrogen, oxygen or optionally oxidized sulfur; said ring is substituted with at least one substituent R³;
s3 is 0, 1, 2, 3, 4 or 5;
R¹, R², and R³ are each independently hydrogen, halogen, —C₁-C₈alkyl, —C₃-C₈cycloalkyl, 3- to 8-membered heterocyclyl, C₆-C₁₂aryl, 5- to 12-membered heteroaryl, oxo(═O), —CN, —OR^{1a}, —C(O)R^{1a}—, —CO₂R^{1a}, —C(O)NR^{1a}R^{1b}, —NR^{1a}R^{1b}, —NR^{1a}COR^{1b} or —NR^{1a}CO₂R^{1b}; each of —C₁-C₈alkyl, C₃-C₈cycloalkyl, 3- to 8-membered heterocyclyl, C₆-C₁₂aryl or 5- to 12-membered heteroaryl is optionally substituted with at least one substituent R^{1c}; or
when adjacent, two R¹, two R² or two R³ together with the atoms to which they are attached, form a 3- to 12-membered ring, said ring comprising 0-3 heteroatoms independently selected from nitrogen, oxygen or sulfur; said ring is optionally substituted with at least one substituent R^{1c};
   R^{1a} and R^{1b} are each independently selected from hydrogen, —C₁-C₈alkyl, —C₁-C₈haloalkyl, —C₂-C₈alkenyl, —C₂-C₈alkynyl, C₁-C₈alkoxy-C₁-C₈alkyl-, C₃-C₈cycloalkyl, 3- to 8-membered heterocyclyl, C₆-C₁₂aryl or 5- to 12-membered heteroaryl; each of —C₁-C₈alkyl, —C₁-C₈haloalkyl, —C₂-C₈alkenyl, —C₂-C₈alkynyl, C₁-C₈alkoxy-C₁-C₈alkyl-, C₃-C₈cycloalkyl, 3- to 8-membered heterocyclyl, C₆-C₁₂aryl or 5- to 12-membered heteroaryl is optionally substituted with at least one substituent selected from halogen, —OH, —CN, oxo (═O), —C₁-C₈alkyl, —C₁-C₈alkyl-OH, —C₂—C₈alkenyl, —C₂-C₈alkynyl, —C₃-C₈cycloalkyl, 3- to 8-membered heterocyclyl, —C₆-C₁₂aryl, or 5- to 12-membered heteroaryl;
   R^{1c}, at each occurrence, is independently halogen, —OH, —CN, oxo (═O), —C₁-C₈alkyl, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;
R^{11a}, R^{11b}, R^{11c}, R^{11d}, R^{12a}, R^{12b}, R^{12c} and R^{12d} are each independently absent, oxo, hydrogen, halogen, —C₁₋₈alkyl, —C₂₋₈alkenyl, —C₂₋₈alkynyl, —C₁₋₈alkoxy or —C₃₋₈cycloalkyl; each of said —C₁₋₈alkyl, —C₂₋₈alkenyl, —C₂₋₈alkynyl, —C₁₋₈alkoxy or —C₃₋₈cycloalkyl is optionally substituted with at least one substituent selected from hydrogen, halogen, —C₁₋₈alkyl, —C₂₋₈alkenyl, —C₂₋₈alkynyl, —C₁₋₈alkoxy or —CN; or
   R^{11a} and R^{12a} together with the carbon atoms to which they are attached, form a 3- to 12-membered ring, said ring comprising 0-3 heteroatoms independently selected from nitrogen, oxygen or sulfur; said ring is optionally substituted with at least one substituent selected from hydrogen, halogen, —C₁₋₈alkyl, —C₂₋₈alkenyl, —C₂₋₈alkynyl, —C₁₋₈alkoxy or —CN; and/or
   R^{11b} and R^{12b} together with the carbon atoms to which they are attached, form a 3- to 12-membered ring, said ring comprising 0-3 heteroatoms independently selected from nitrogen, oxygen or sulfur; said ring is optionally substituted with at least one substituent selected from hydrogen, halogen, —C₁₋₈alkyl, —C₂₋₈alkenyl, —C₂₋₈alkynyl, —C₁₋₈alkoxy or —CN; and/or
   R^{11c} and R^{12d} together with the carbon atoms to which they are attached, form a 3- to 12-membered ring, said ring comprising 0-3 heteroatoms independently selected from nitrogen, oxygen or sulfur; said ring is optionally substituted with at least one substituent selected from hydrogen, halogen, —C₁₋₈alkyl, —C₂₋₈alkenyl, —C₂₋₈alkynyl, —C₁₋₈alkoxy or —CN; and/or
   R^{11d} and R^{12d} together with the carbon atoms to which they are attached, form a 3- to 12-membered ring, said ring comprising 0-3 heteroatoms independently selected from nitrogen, oxygen or sulfur; said ring is optionally substituted with at least one substituent selected from hydrogen, halogen, —C₁₋₈alkyl, —C₂₋₈alkenyl, —C₂₋₈alkynyl, —C₁₋₈alkoxy or —CN; each of occurrence, L¹ is independently selected from —O—, —NR^{a}—, —C(O)NR^{a}—, —C(O)O—,

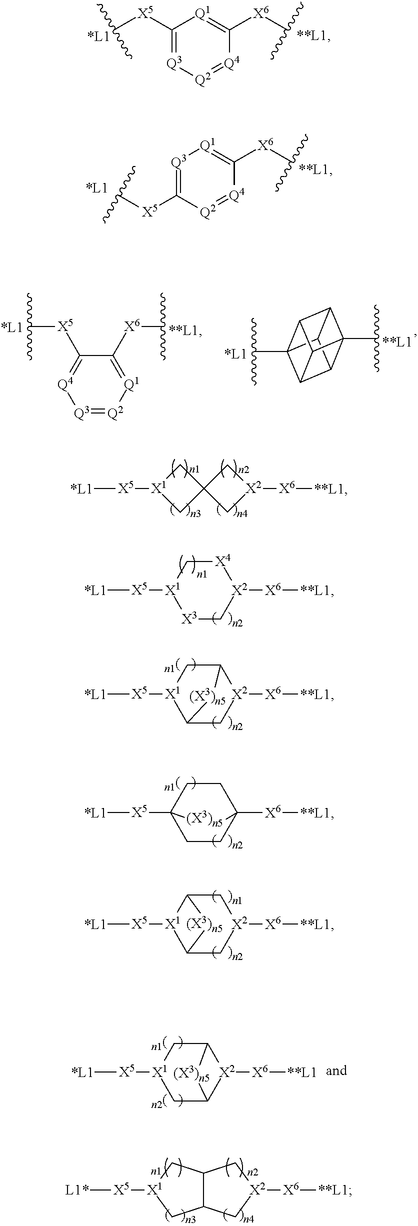

wherein each of said

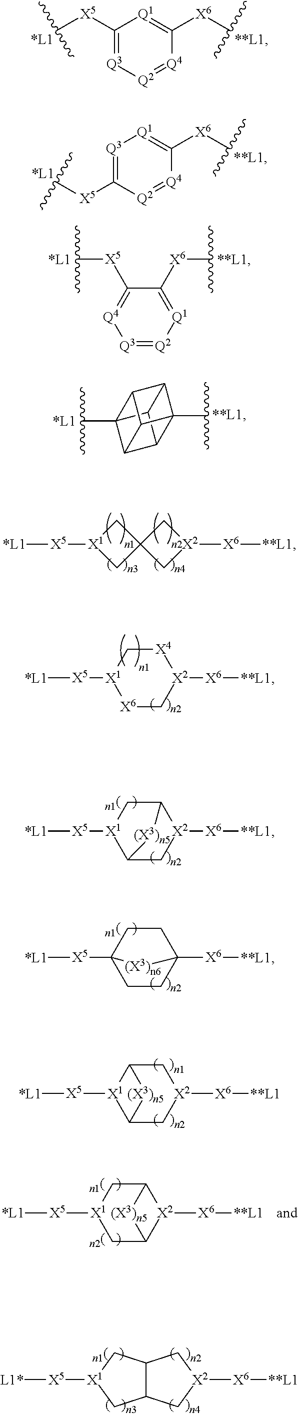

is optionally substituted with at least one R^{L1c};
wherein *^{L1} refers to the position attached to the

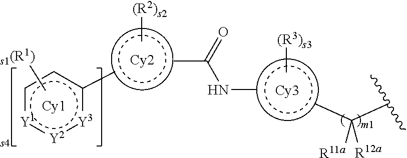

moiety, and **^{L1} refers to the position attached to the

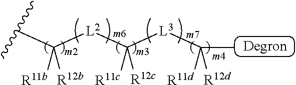

moiety;
each of occurrence, L² is independently selected from —O—, —NR^{a}—, —CONR^{a}—, —C(O)O—,

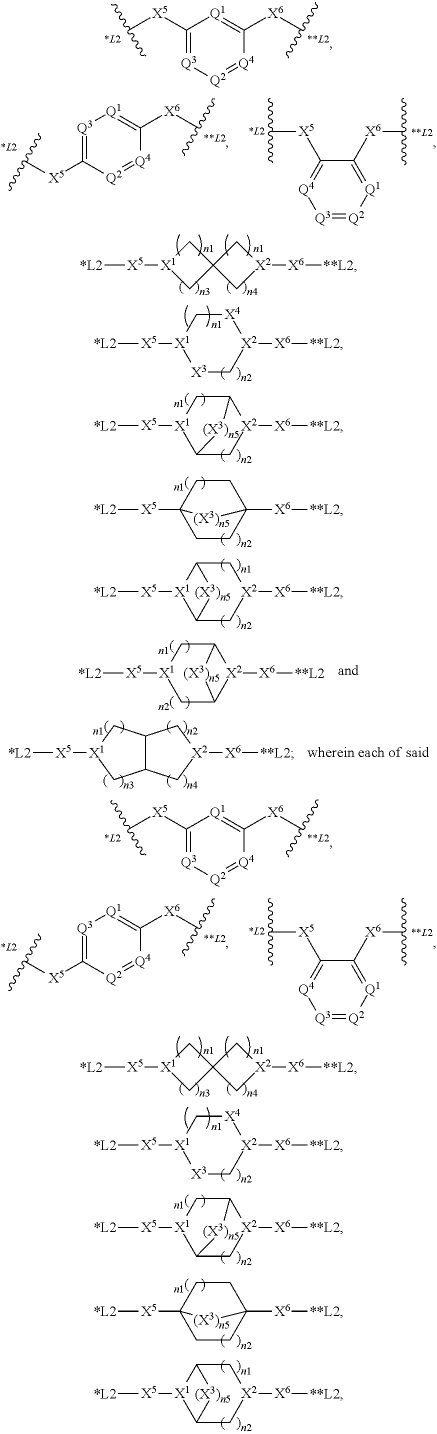

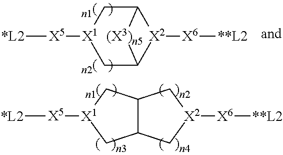

is optionally substituted with at least one R^{L2c};
wherein *L² refers to the position attached to the

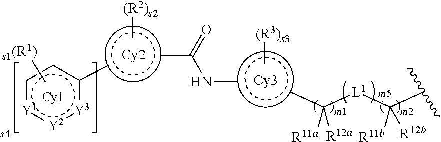

moiety, and **^{L2} refers to the position attached to the

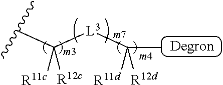

moiety;
each of occurrence, L³ is independently selected from —O—, —NR^{a}—, —CONR^{a}—, —C(O)O—,

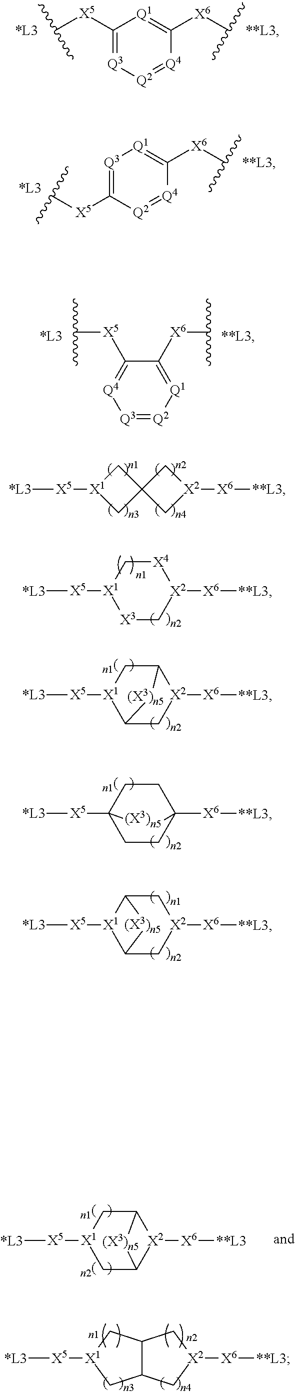

wherein each of said

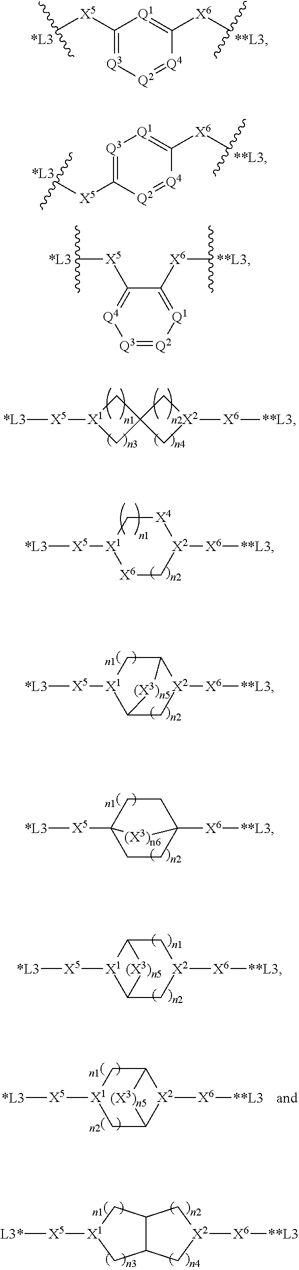

is optionally substituted with at least one R^{L3c};
wherein *^{L3} refers to the position attached to the

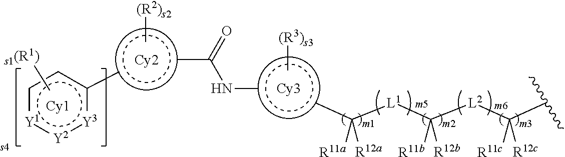

moiety, and **^{L3} refers to the position attached to the

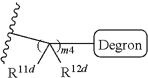

moiety;
each of said R^{L1c}, R^{L2c} and R^{L3c} are independently absent, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; each of said —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl and 5- to 12-membered heteroaryl is optionally substituted with at least one R^{Lca},
R^{Lca} is independently absent, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or
two R^{L1c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L2c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L3c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
[symbol] is selected from

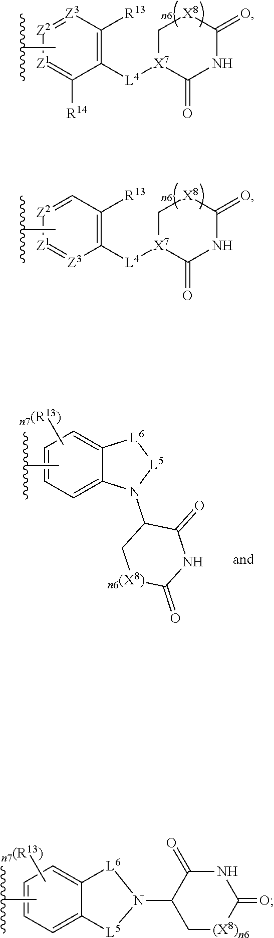

Z¹, Z² and Z³ are each independently N or CR^{z}, provided that Z¹, Z² and Z³ are not N at the same time;
R^{z}, at each occurrence, is independently selected from absent, hydrogen, halogen, —C₁₋₈alkyl, —NR^{Za}R^{Zb}, —OR^{Za}, SR^{Za} C₃-C₈cycloalkyl, 3- to 8-membered heterocyclyl or CN; each of —C₁₋₈alkyl, C₃-C₈cycloalkyl, 3- to 8-membered heterocyclyl is optionally substituted with at least one R^{Zc};
the

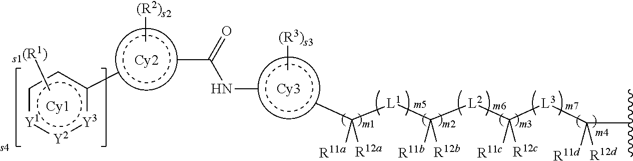

moiety is linked to

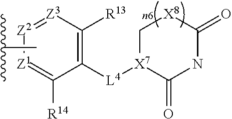

moiety via any one of Z¹, Z² or Z³ which is CR^{z} and R^{z} is absent;
R^{Za} and R^{Zb} are each independently selected from absent, hydrogen, —C₁-C₈alkyl, C₃-C₈cycloalkyl, 3- to 8-membered heterocyclyl, C₆-C₁₂aryl, or 5- to 12-membered heteroaryl, each of said —C₁₋₈alkyl, C₃-C₈cycloalkyl, 3- to 8-membered heterocyclyl, C₆-C₁₂aryl, or 5- to 12-membered heteroaryl is optionally substituted with at least one substituent R^{Zd};
R^{Zc} and R^{Zd} are each independently halogen, hydroxy, —C₁-C₈alkyl, —C₁₋₈alkoxy, C₃-C₈cycloalkyl, 3- to 8-membered heterocyclyl, C₆-C₁₂aryl, or 5- to 12-membered heteroaryl;
R¹³ and R¹⁴ are each independently selected from absent, hydrogen, halogen, —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}, —NR^{13a}CO₂R^{13b} or —NR^{13a}SO₂R^{13b}; each of —C₁₋₈alkyl, —C₂₋₈alkenyl, —C₂₋₈alkynyl, —C₁₋₈ alkoxy, —C₃-C₈cycloalkyl, 3- to 8-membered heterocyclyl, —C₆-C₁₂aryl or 5- to 12-membered heteroaryl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{13c}, —CONR^{13c}R^{13a}, —NR^{13c}R^{13d}, —NR^{13c}COR^{13d}, —NR^{13c}CO₂R^{13a}, or —NR^{13c}SO₂R^{13d};
at each occurrence, R^{13a}, R^{13b}, R^{13c} and R^{13d} are each independently absent, hydrogen, —C₁₋₈alkyl, —C₂-8alkenyl, —C₂₋₈alkynyl, C₃-C₈cycloalkyl, 3- to 8-membered heterocyclyl, C₆-C₁₂aryl, or 5- to 12-membered heteroaryl;
L⁴, L⁵ and L⁶ are each independently selected from a absent, single bond, —O—, —NR^{a}—, —(CR^{a}R^{b})ₙ₈—, —O(CR^{a}R^{b})ₙ₈—, —NR^{a}(CR^{a}R^{b})ₙ₈— or —C(O)—;
at each occurrence, X¹, X² and X⁷ are each independently selected from —CR^{a}, or N;
at each occurrence, X³, X⁴ and X⁸ are each independently selected from —NR^{a}—, —O—, —S— and —CR^{a}R^{b}—;
at each occurrence, X⁵ and X⁶ are each independently selected from absent, single bond, —C(O)—, —NR^{a}— and —O—;
Q¹, Q², Q³ and Q⁴ are each independently selected from CR^{a} or N;
at each occurrence, R^{a} and R^{b} are each independently selected from hydrogen, hydroxy, halogen, CN, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl, each of said —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl is optionally substituted with at least one substituent halogen, hydroxy, halogen, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; or
R^{a} and R^{b} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl, —C₂-C₈alkenyl, —C₂-C₈alkynyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl;
m1, m2, m3 and m4 are each independently 0, 1 or 2;
m5 and m7 are each independently 0 or 1;
m6 is 0, 1 or 2; when m6 is 2, two L2 are same or different;
n1, n2, n3, n4 and n5 are each independently 0, 1, 2 or 3;
n6, n7 and n8 are each independently 0, 1, 2, 3 or 4.

Aspect 3. A compound of formula (I′)

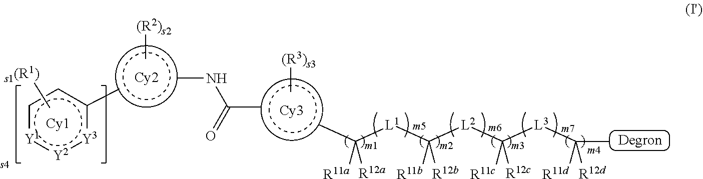

or a N-oxide thereof, or a pharmaceutically acceptable salt thereof, or a stereoisomer thereof, or a deuterated analog thereof, or a prodrug thereof,
wherein:
Cy1 is a 6-membered partially or completely unsaturated ring; said ring is substituted with at least one substituent R¹;
Y¹, Y² and Y³ are each independently C or N;
s1 is 0, 1, 2, 3, 4 or 5;
Cy2 is a 5-, 6-, 7-, 8-, 9- or 10-membered partially or completely unsaturated ring (aromatic ring); said ring comprising 0-3 heteroatoms independently selected from nitrogen, oxygen or optionally oxidized sulfur; said ring is substituted with at least one substituent R²;
s2 is 0, 1 or 2;
s4 is 0 or 1;
Cy3 is a 5-, 6-, 7-, 8-, 9- or 10-membered aromatic ring; said ring comprising 0-3 heteroatoms independently selected from nitrogen, oxygen or optionally oxidized sulfur; said ring is substituted with at least one substituent R³;
s3 is 0, 1, 2, 3, 4 or 5;
R¹, R², and R³ are each independently hydrogen, halogen, —C₁-C₈alkyl, —C₃-C₈cycloalkyl, 3- to 8-membered heterocyclyl, C₆-C₁₂aryl, 5- to 12-membered heteroaryl, oxo(═O), —CN, —OR^{1a}, —C(O)R^{1a}, —CO₂R^{1a}, —C(O)NR^{1a}R^{1b}, —NR^{1a}R^{1b}, —NR^{1a}COR^{1b} or —NR^{1a}CO₂R^{1b}; each of —C₁-C₈alkyl, C₃-C₈cycloalkyl, 3- to 8-membered heterocyclyl, C₆-C₁₂aryl or 5- to 12-membered heteroaryl is optionally substituted with at least one substituent R^{1c}; or
when adjacent, two R¹, two R² or two R³ together with the atoms to which they are attached, form a 3- to 12-membered ring, said ring comprising 0-3 heteroatoms independently selected from nitrogen, oxygen or sulfur; said ring is optionally substituted with at least one substituent R^{1c};
   R^{1a} and R^{1b} are each independently selected from hydrogen, —C₁-C₈alkyl, —C₁-C₈haloalkyl, —C₂-C₈alkenyl, —C₂-C₈alkynyl, C₁-C₈alkoxy-C₁-C₈alkyl-, C₃-C₈cycloalkyl, 3- to 8-membered heterocyclyl, C₆-C₁₂aryl or 5- to 12-membered heteroaryl; each of —C₁-C₈alkyl, —C₁-C₈haloalkyl, —C₂-C₈alkenyl, —C₂-C₈alkynyl, C₁-C₈alkoxy-C₁-C₈alkyl-, C₃-C₈cycloalkyl, 3- to 8-membered heterocyclyl, C₆-C₁₂aryl or 5- to 12-membered heteroaryl is optionally substituted with at least one substituent selected from halogen, —OH, —CN, oxo (═O), —C₁-C₈alkyl, —C₁-C₈alkyl-OH, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;
   R^{1c}, at each occurrence, is independently halogen, —OH, —CN, oxo (═O), —C₁-C₈alkyl, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;
R^{11a}, R^{11b}, R^{11c}, R^{11d}, R^{12a}, R^{12b}, R^{12c} and R^{12d} are each independently absent, oxo, hydrogen, halogen, —C₁₋₈alkyl, —C₂₋₈alkenyl, —C₂₋₈alkynyl, —C₁₋₈alkoxy or —C₃₋₈cycloalkyl; each of said —C₁₋₈alkyl, —C₂₋₈alkenyl, —C₂₋₈alkynyl, —C₁₋₈alkoxy or —C₃₋₈cycloalkyl is optionally substituted with at least one substituent selected from hydrogen, halogen, —C₁₋₈alkyl, —C₂₋₈alkenyl, —C₂₋₈alkynyl, —C₁₋₈alkoxy or —CN; or
   R^{11a} and R^{12a} together with the carbon atoms to which they are attached, form a 3- to 12-membered ring, said ring comprising 0-3 heteroatoms independently selected from nitrogen, oxygen or sulfur; said ring is optionally substituted with at least one substituent selected from hydrogen, halogen, —C₁₋₈alkyl, —C₂₋₈alkenyl, —C₂₋₈alkynyl, —C₁₋₈alkoxy or —CN; and/or
   R^{11b} and R^{12b} together with the carbon atoms to which they are attached, form a 3- to 12-membered ring, said ring comprising 0-3 heteroatoms independently selected from nitrogen, oxygen or sulfur; said ring is optionally substituted with at least one substituent selected from hydrogen, halogen, —C₁₋₈alkyl, —C₂₋₈alkenyl, —C₂₋₈alkynyl, —C₁₋₈alkoxy or —CN; and/or
   R^{11c} and R^{12c} together with the carbon atoms to which they are attached, form a 3- to 12-membered ring, said ring comprising 0-3 heteroatoms independently selected from nitrogen, oxygen or sulfur; said ring is optionally substituted with at least one substituent selected from hydrogen, halogen, —C₁₋₈alkyl, —C₂₋₈alkenyl, —C₂₋₈alkynyl, —C₁₋₈alkoxy or —CN; and/or
   R^{11d} and R^{12d} together with the carbon atoms to which they are attached, form a 3- to 12-membered ring, said ring comprising 0-3 heteroatoms independently selected from nitrogen, oxygen or sulfur; said ring is optionally substituted with at least one substituent selected from hydrogen, halogen, —C₁₋₈alkyl, —C₂₋₈alkenyl, —C₂₋₈alkynyl, —C₁₋₈alkoxy or —CN;
each of occurrence, L¹ is independently selected from —O—, —NR^{a}—, —C(O)NR^{a}—, —C(O)O—,

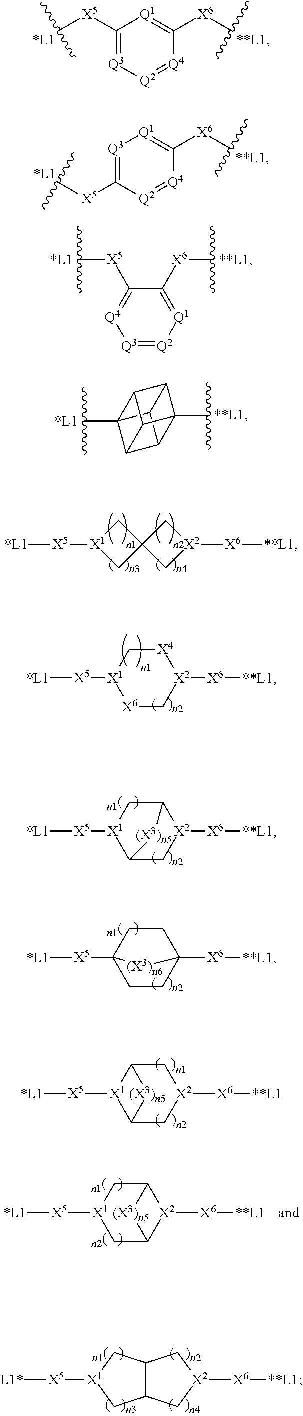

wherein each of said

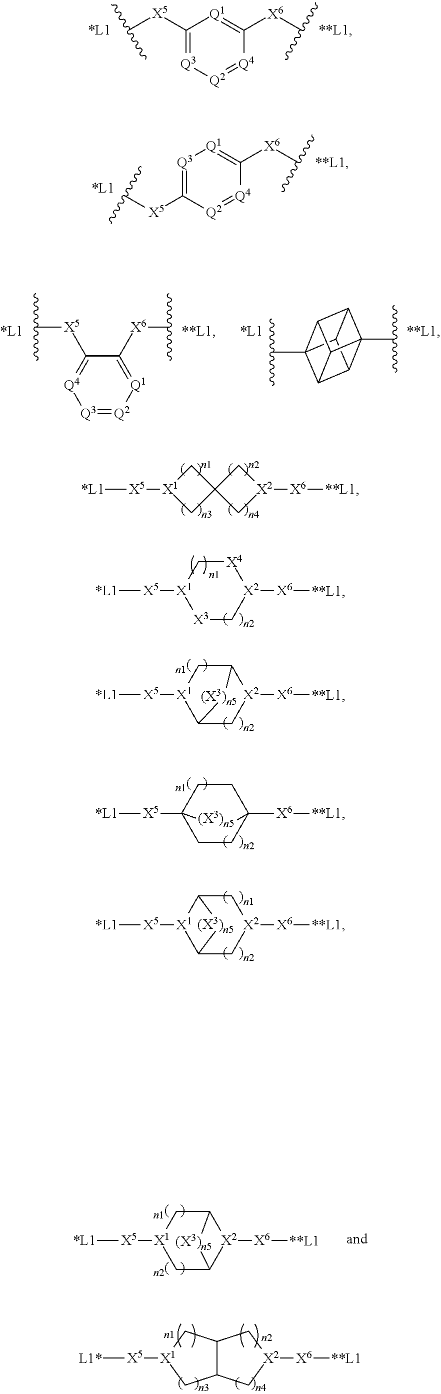

is optionally substituted with at least one R^{L1c};
wherein *^{L1} refers to the position attached to the

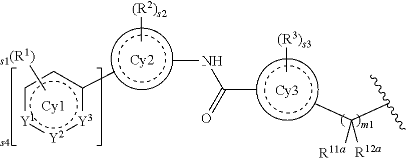

moiety, and **^{L1} refers to the position attached to the

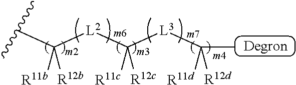

moiety;
each of occurrence, L² is independently selected from —O—, —NR^{a}—, —CONR^{a}—, —C(O)O—,

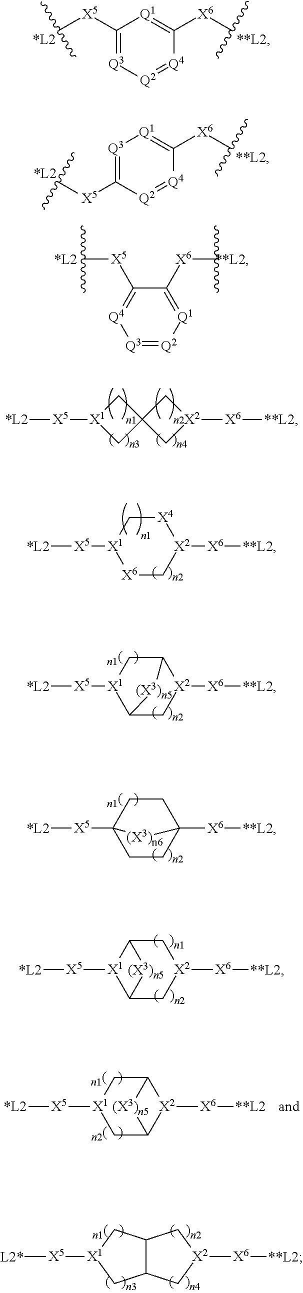

wherein each of said

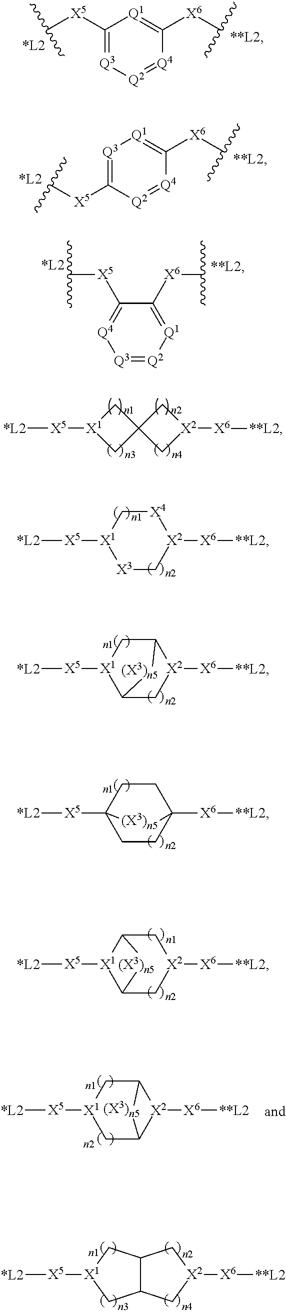

is optionally substituted with at least one R^{L2c};
wherein *^{L2} refers to the position attached to the

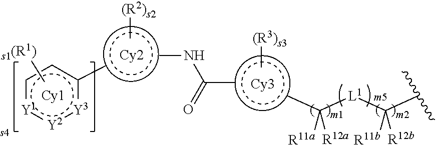

moiety, and **^{L2} refers to the position attached to the

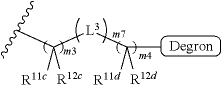

moiety;
each of occurrence, L³ is independently selected from —O—, —NR^{a}—, —CONR^{a}—, —C(O)O—,

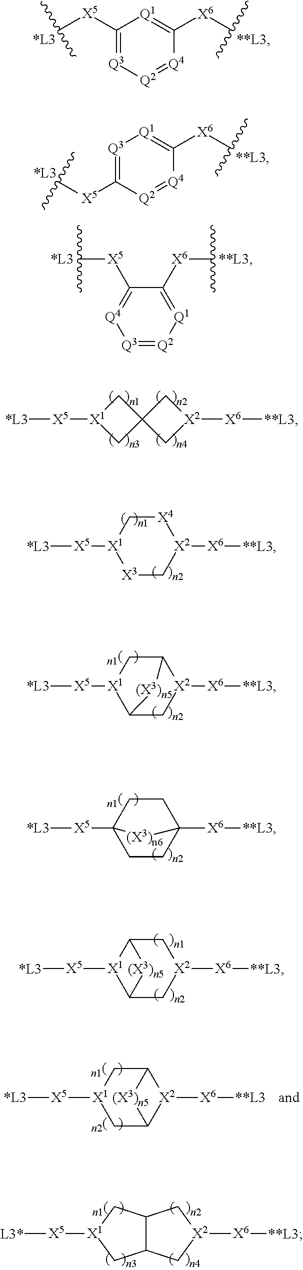

wherein each of said

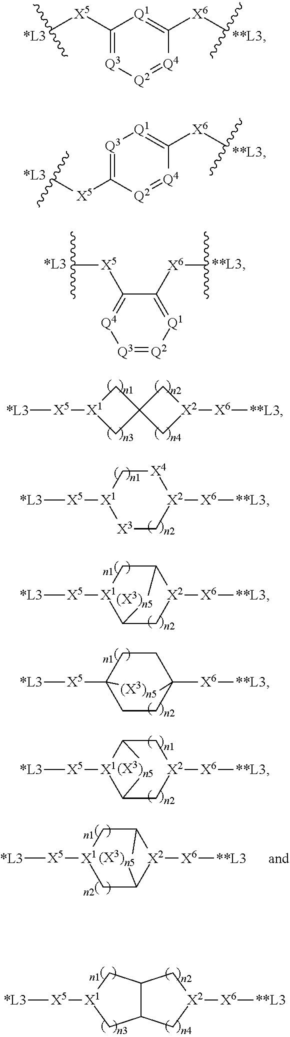

is optionally substituted with at least one R^{L3c};
wherein *^{L3} refers to the position attached to the

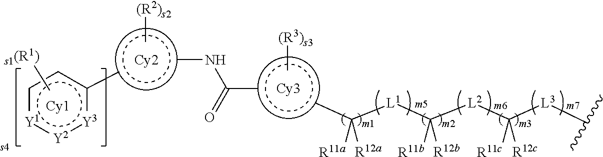

moiety, and **^{L3} refers to the position attached to the

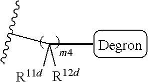

moiety;
each of said R^{L1c}, R^{L2c} and R^{L3c} are independently absent, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; each of said —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl and 5- to 12-membered heteroaryl is optionally substituted with at least one R^{Lca},
R^{Lca} is independently absent, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or
two R^{L1c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L2c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L3c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl; [symbol] is selected from

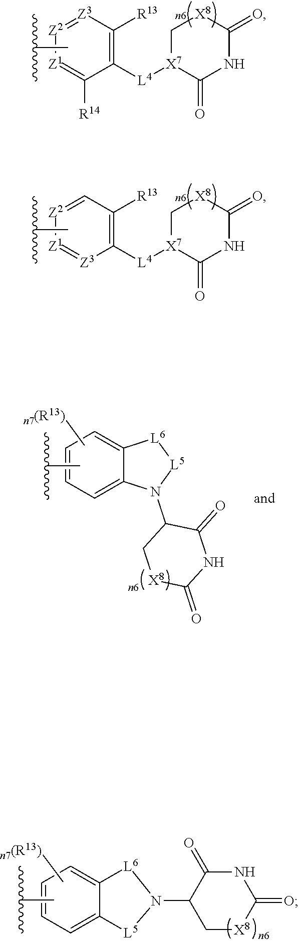

Z¹, Z² and Z³ are each independently N or CR^{z}, provided that Z¹, Z² and Z³ are not N at the same time;
R^{z}, at each occurrence, is independently selected from absent, hydrogen, halogen, —C₁₋₈alkyl, —NR^{Za}R^{Zb}, —OR^{Za}, SR^{Za} C₃-C₈cycloalkyl, 3- to 8-membered heterocyclyl or CN; each of —C₁₋₈alkyl, C₃-C₈cycloalkyl, 3- to 8-membered heterocyclyl is optionally substituted with at least one R^{Zc};
the

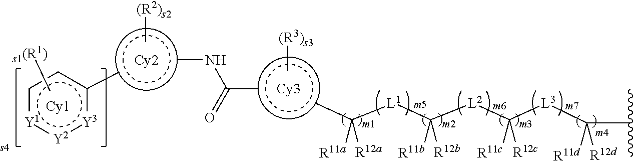

moiety is linked to the

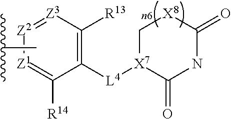

moiety via any one of Z¹, Z² or Z³ which is CR^{z} and R^{z} is absent;
R^{Za} and R^{Zb} are each independently selected from absent, hydrogen, —C₁-C₈alkyl, C₃-C₈cycloalkyl, 3- to 8-membered heterocyclyl, C₆-C₁₂aryl, or 5- to 12-membered heteroaryl, each of said —C₁₋₈alkyl, C₃-C₈cycloalkyl, 3- to 8-membered heterocyclyl, C₆-C₁₂aryl, or 5- to 12-membered heteroaryl is optionally substituted with at least one substituent R^{Zd};
R^{Zc} and R^{Zd} are each independently halogen, hydroxy, —C₁-C₈alkyl, —C₁₋₈alkoxy, C₃-C₈cycloalkyl, 3- to 8-membered heterocyclyl, C₆-C₁₂aryl, or 5- to 12-membered heteroaryl;
R¹³ and R¹⁴ are each independently selected from absent, hydrogen, halogen, —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, CONR^{13a}R^{13b}, —NR^{13a}R^{13b}, —NR^{13a}COR^{13b}, —NR^{13a}CO₂R^{13b}, or —NR^{13a}SO₂R^{13b}; each of —C₁₋₈alkyl, —C₂₋₈alkenyl, —C₂₋₈alkynyl, —C₁₋₈ alkoxy, —C₃-C₈cycloalkyl, 3- to 8-membered heterocyclyl, —C₆-C₁₂aryl or 5- to 12-membered heteroaryl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{13c}, —CONR^{13c}R^{13d}, —NR^{13c}R^{13d}, —NR^{13c}COR^{13d}, —NR^{13c}CO₂R^{13a}, or —NR^{13c}SO₂R^{13d};
at each occurrence, R^{13a}, R^{13b}, R^{13c} and R^{13d} are each independently absent, hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl, or 5- to 12-membered heteroaryl;
L⁴, L⁵ and L⁶ are each independently selected from a absent, single bond, —O—, —NR^{a}—, —(CR^{a}R^{b})ₙ₈—, —O(CR^{a}R^{b})ₙ₈—, —NR^{a}(CR^{a}R^{b})ₙ₈— or —C(O)—;
at each occurrence, X¹, X² and X⁷ are each independently selected from —CR^{a}, or N;
at each occurrence, X³, X⁴ and X⁸ are each independently selected from —NR^{a}—, —O—, —S— and —CR^{a}R^{b}—;
at each occurrence, X⁵ and X⁶ are each independently selected from absent, single bond, —C(O)—, —NR^{a}— and —O—;
Q¹, Q², Q³ and Q⁴ are each independently selected from CR or N;
at each occurrence, R^{a} and R^{b} are each independently selected from hydrogen, hydroxy, halogen, CN, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl, each of said —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl is optionally substituted with at least one substituent halogen, hydroxy, halogen, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; or
R^{a} and R^{b} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl, —C₂-C₈alkenyl, —C₂-C₈alkynyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl;
m1, m2, m3 and m4 are each independently 0, 1 or 2;
m5 and m7 are each independently 0 or 1;
m6 is 0, 1 or 2; when m6 is 2, two L2 are same or different;
n1, n2, n3, n4 and n5 are each independently 0, 1, 2 or 3;
n6, n7 and n8 are each independently 0, 1, 2, 3 or 4.

Aspect 4. The compound of Aspect 1, wherein the compound is selected from formula (IIa), (IIa′), (IIb), (IIb′), (IIc), (IIc′), (IId), (IId′), (IIe) and (IIe′),

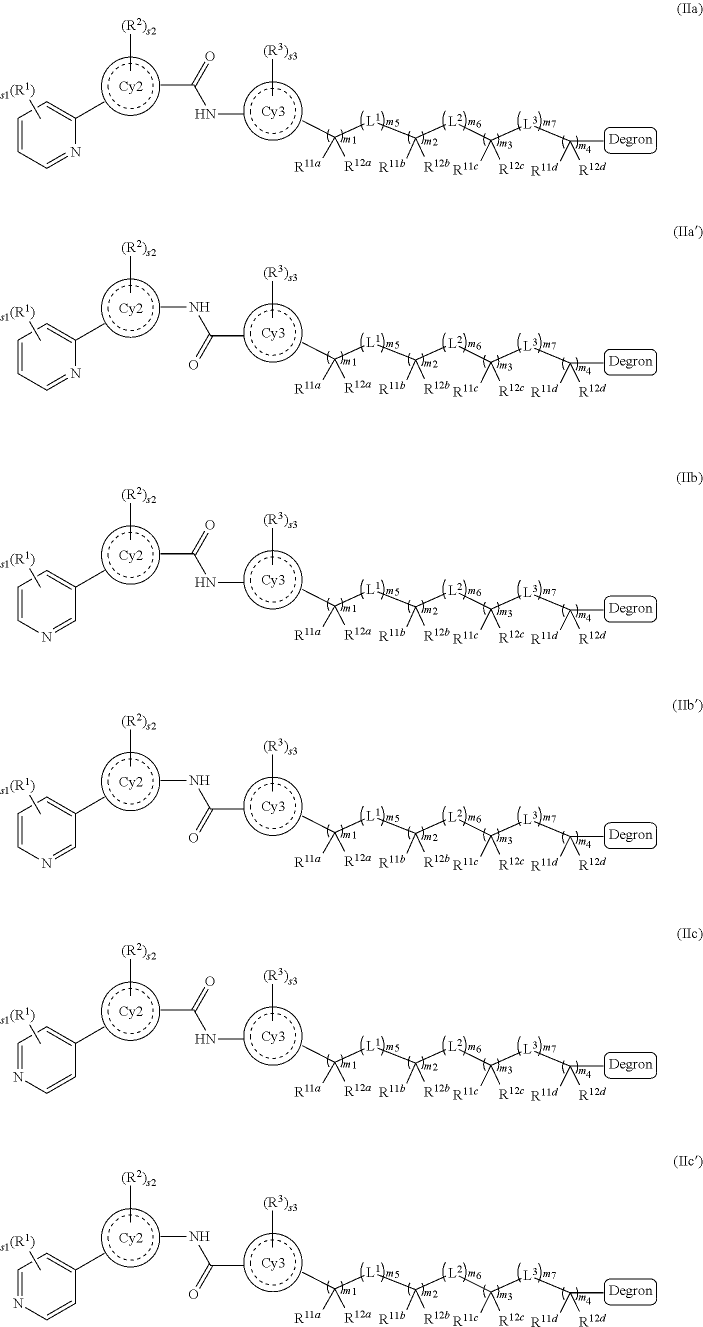

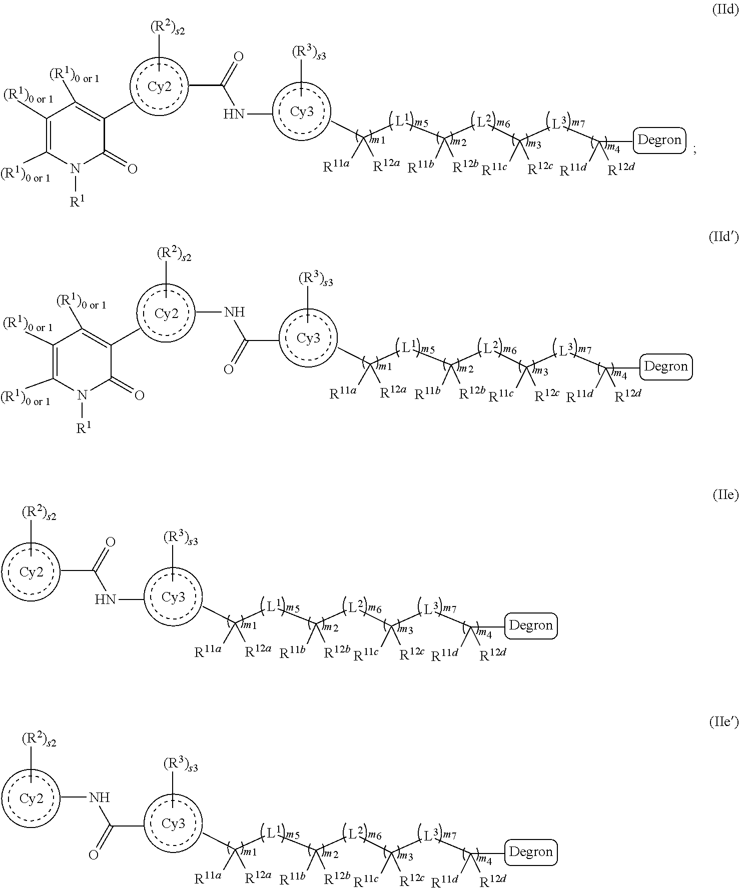

wherein R¹, R², R³, R^{11a}, R^{11b}, R^{11c}, R^{11d}, R^{12a}, R^{12b}, R^{12c}, R^{12d}, L¹, L², L³, s1, s2, s3, m1, m2, m3, m4, m5, m6, m7, Cy2, Cy3, Degron are defined as Aspect 1.

Aspect 5. The compound of Aspect 1, wherein the compound is selected from formula (IIIa), (IIIa′), (IIIb), (IIIb′), (IIIc), (IIIc′), (IIId), (IIId′), (IIIe), (IIIe′), (IIIf), (IIIf′), (IIIg), (IIIg′), (IIIh), (IIIh′), (IIIi), (IIIi′, (IIIj) (IIIj′), (IIIk), (IIIk′), (IIIl) or (IIIl′):

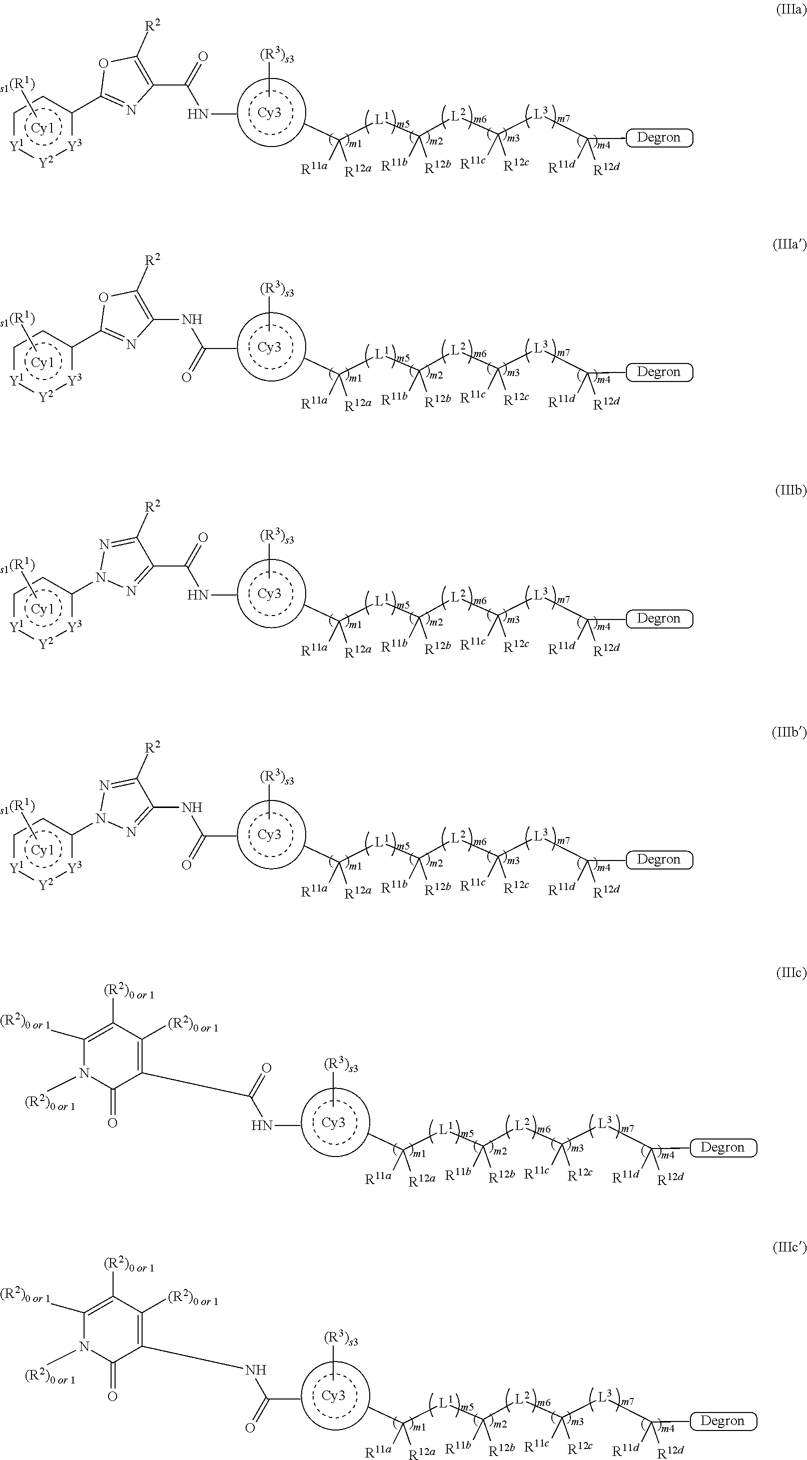

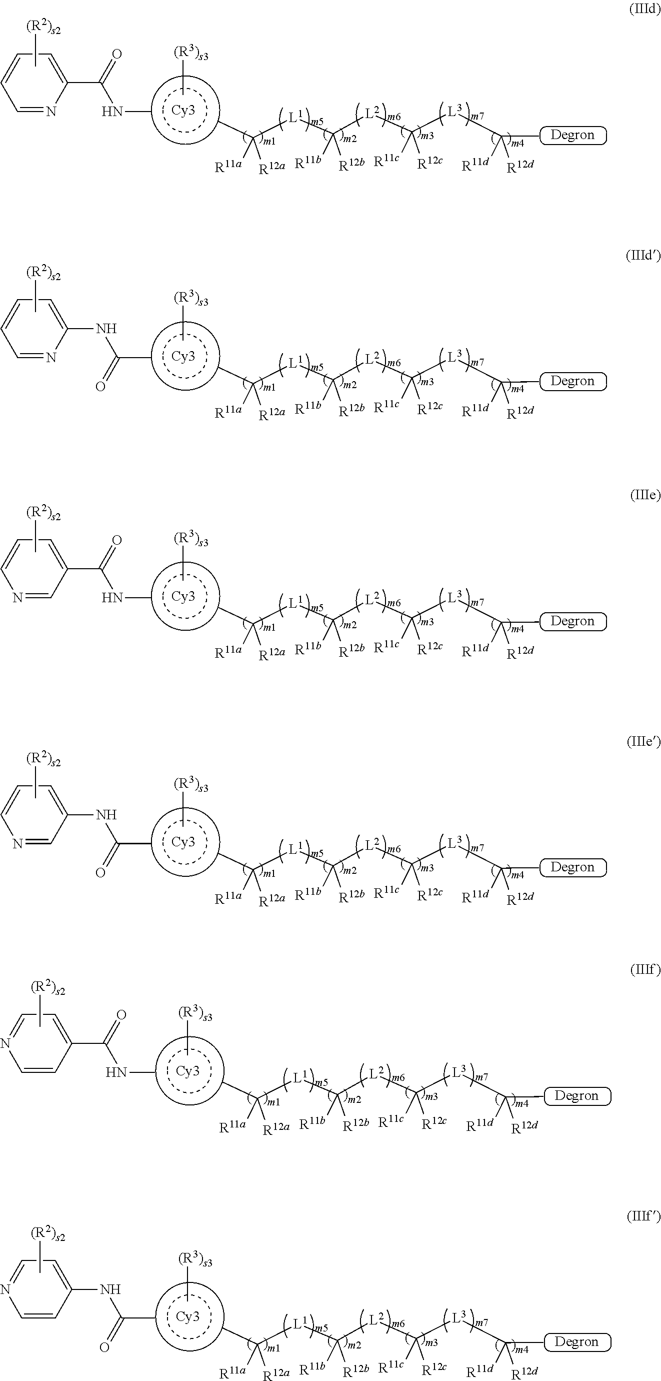

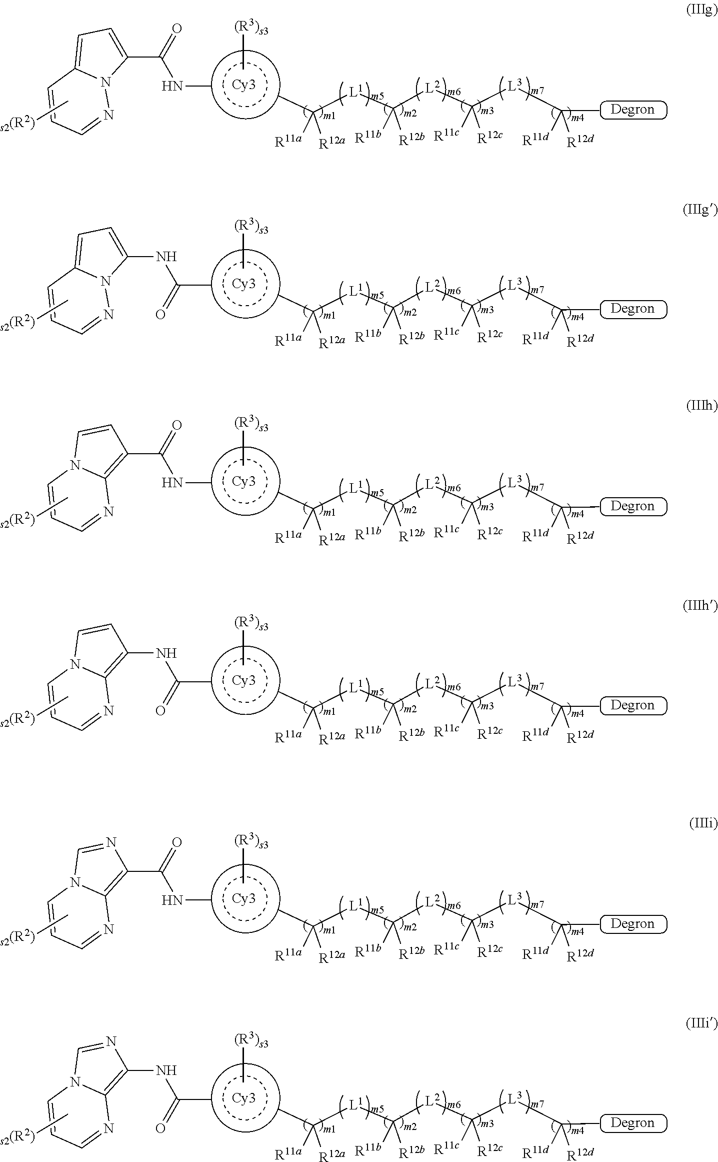

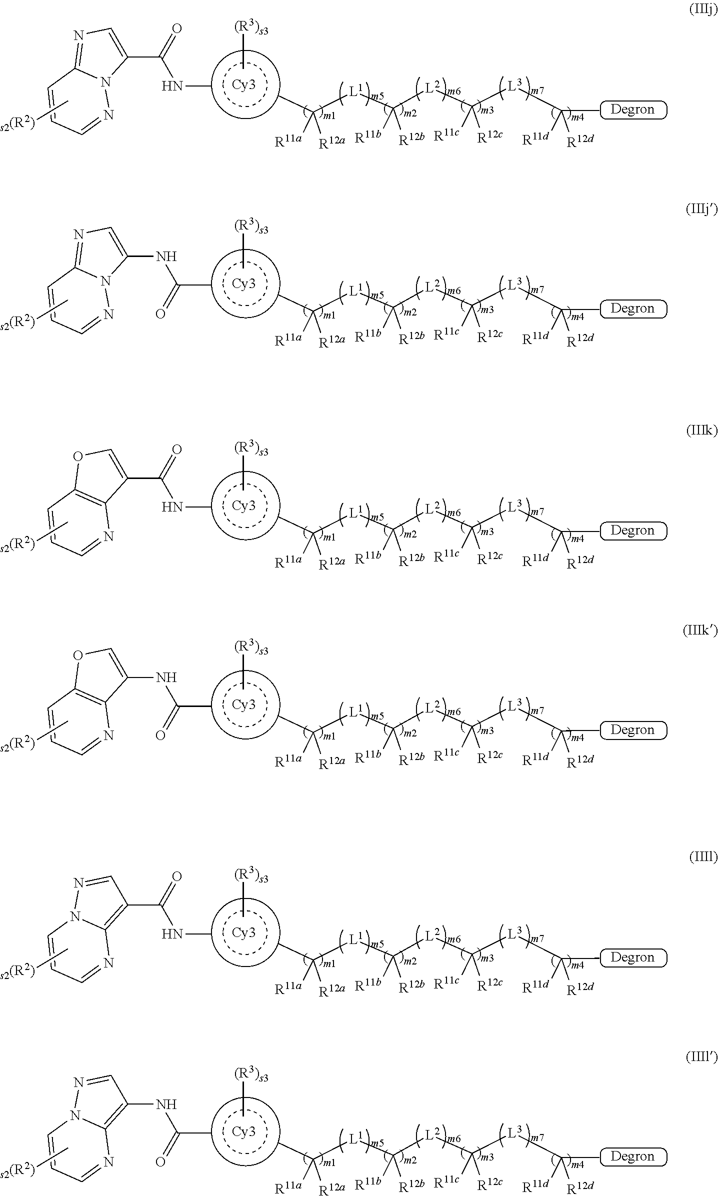

wherein R¹, R², R³, R^{11a}, R^{11b}, R^{11c}, R^{11d}, R^{12a}, R^{12b}, R^{12c}, R^{12d}, Y¹, Y², Y³, L¹, L², L³, s1, s2, s3, m1, m2, m3, m4, m5, m6, m7, Cy1, Cy3, Degron are defined as Aspect 1.

4. The compound of Aspect 1, wherein the compound is selected from formula (IVa), (Iva′), (IVb), (IVb′), (IVc) and (IVc′),

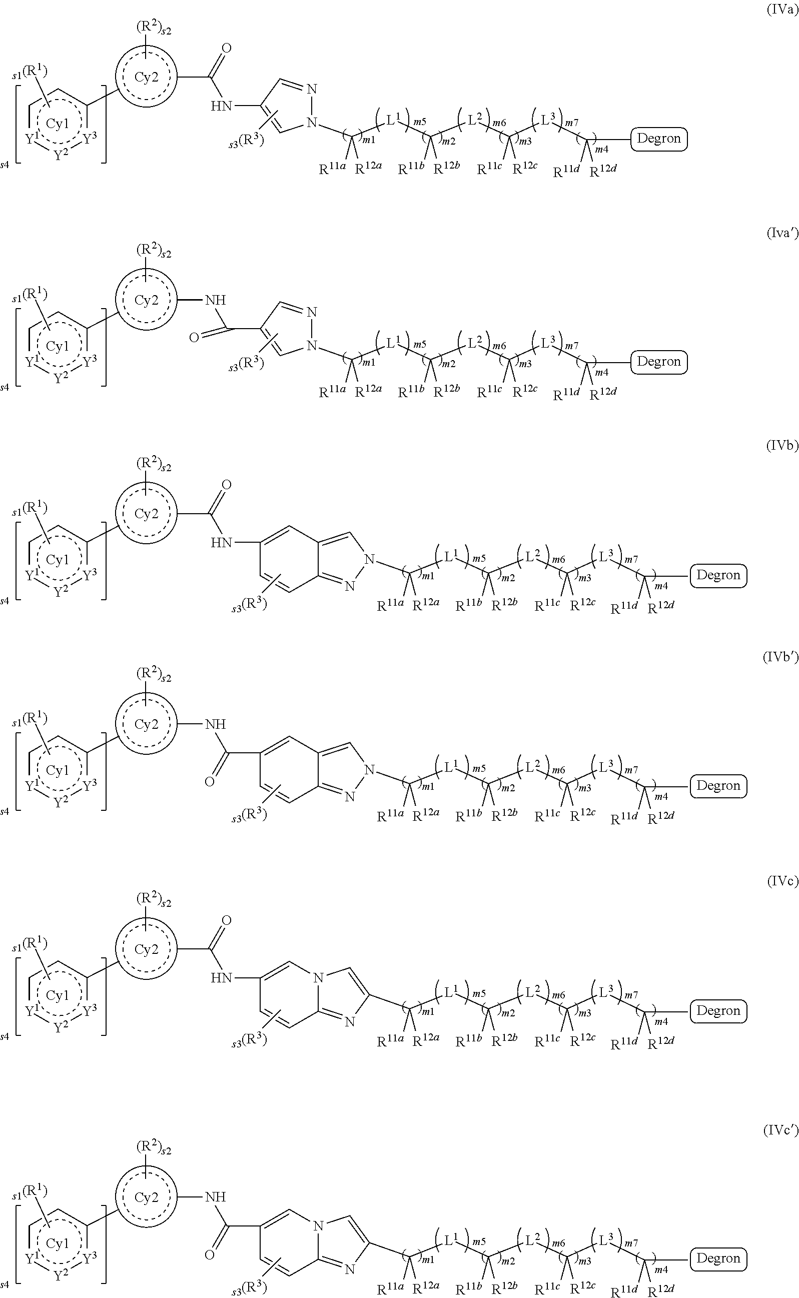

wherein, R¹, R², R³, R^{11a}, R^{11b}, R^{11c}, R^{11d}, R^{12a}, R^{12b}, R^{12c}, R^{12d}, Y¹, Y², Y³, Cy1, Cy2, L¹, L², L³, s1, s2, s3, s4, m1, m2, m3, m4, m5, m6, m7 and Degron are as defined as Aspect 1.

5. The compound of Aspect 1, wherein the compound is selected from formula (Va) or (Va′)

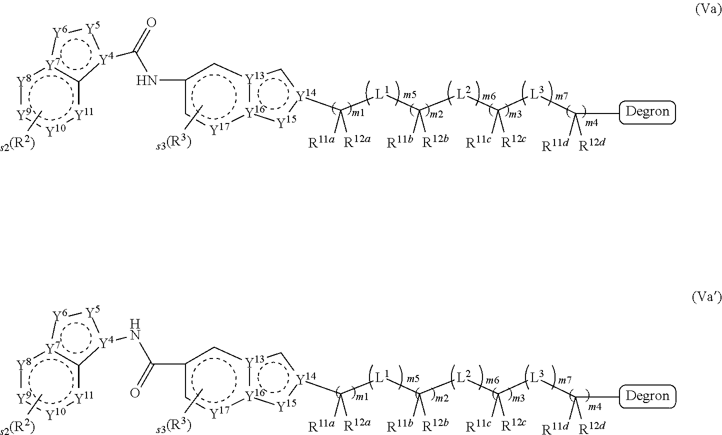

preferably, the compound is selected from formulas (Vb), (Vb′), (Vc) and (Vc′),

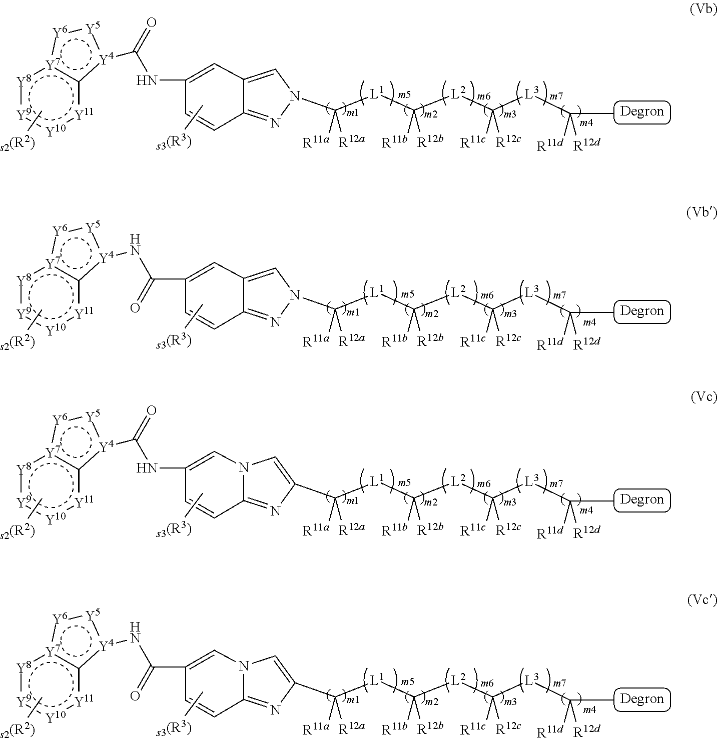

more preferably the compound is selected from formula (Vd) (Vd′), (Ve) and (Ve′),

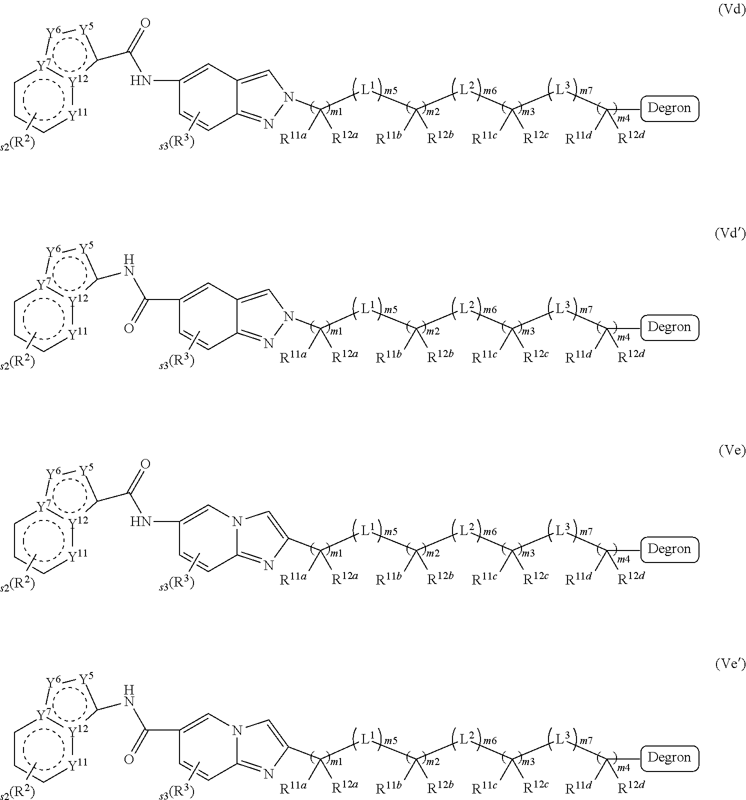

more preferably, the compound is selected from formula (Vf), (Vf′), (Vg), (Vg′), (Vh), (Vh′), (Vi), (Vi′), (Vj), (Vj′), (Vk), (Vk′), (Vl), (Vl′), (Vm), (Vm′), (Vn), (Vn′), (Vo), (Vo′), (Vp), (Vp′), (Vq) or (Vq′),

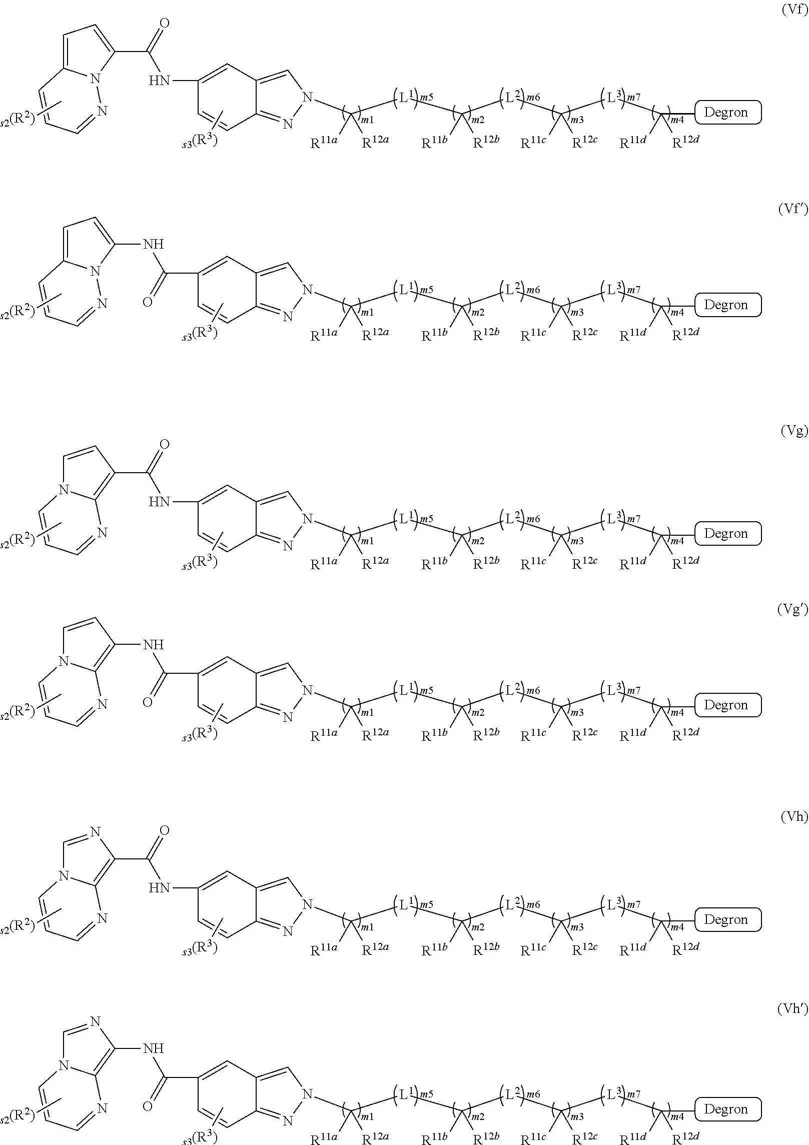

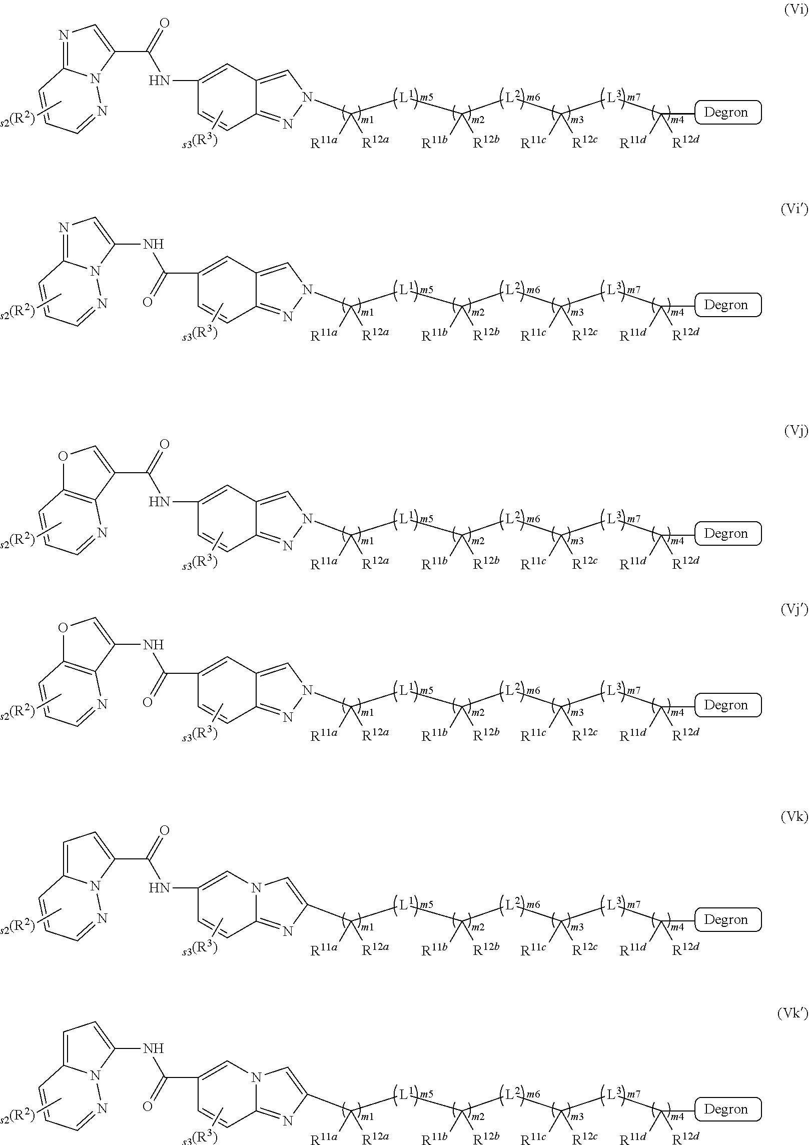

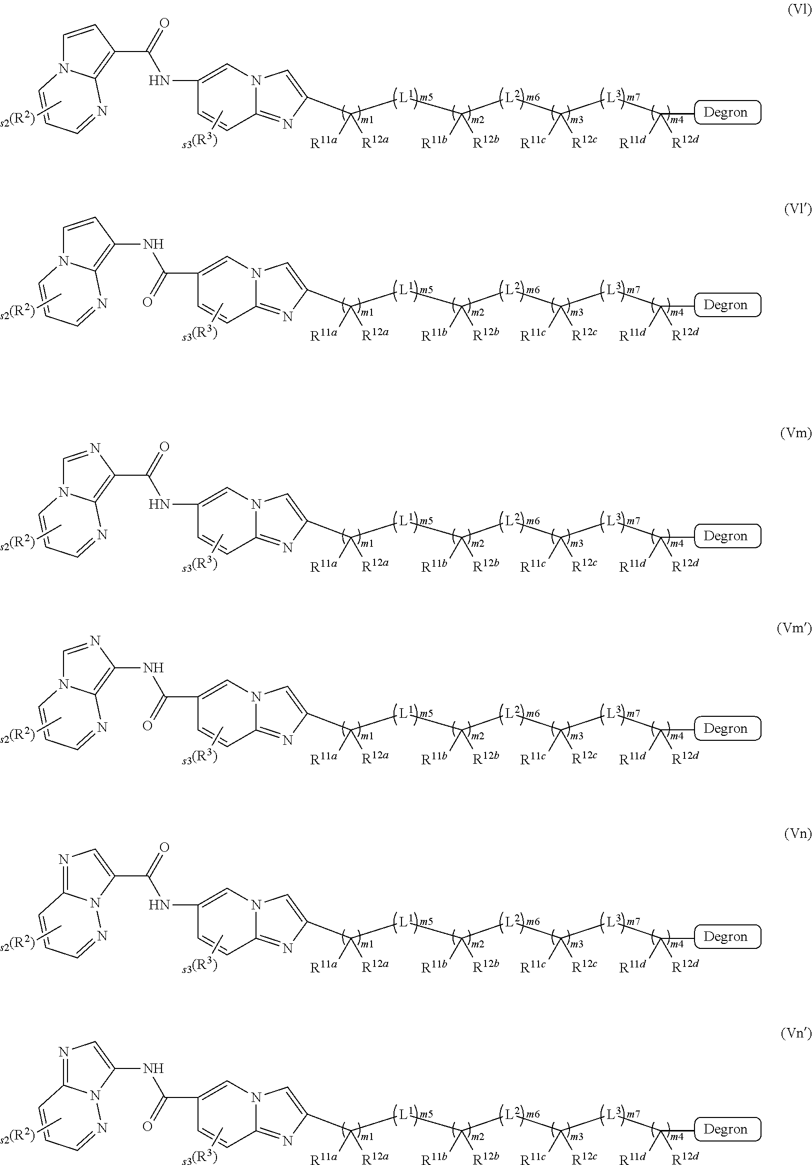

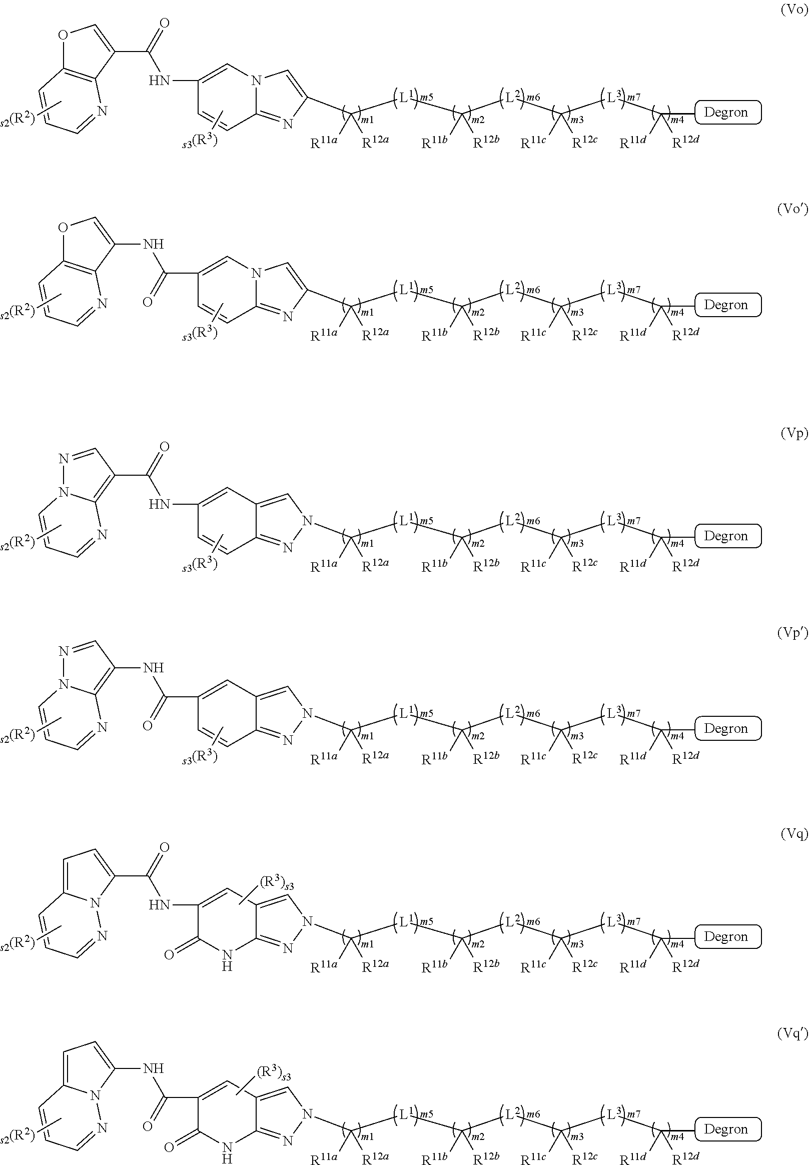

even more preferably, the compound is selected from formula (Vr), (Vr′), (Vs), (Vs′), (Vt), (Vt′), (Vu), (Vu′), (Vv), (Vv′), (Vw), (Vw′), (Vx), (Vx′), (Vy), (Vy′), (Vz), (Vz′), (Vaa), (Vaa′), (Vab), (Vab′), (Vac) or (Vac′),

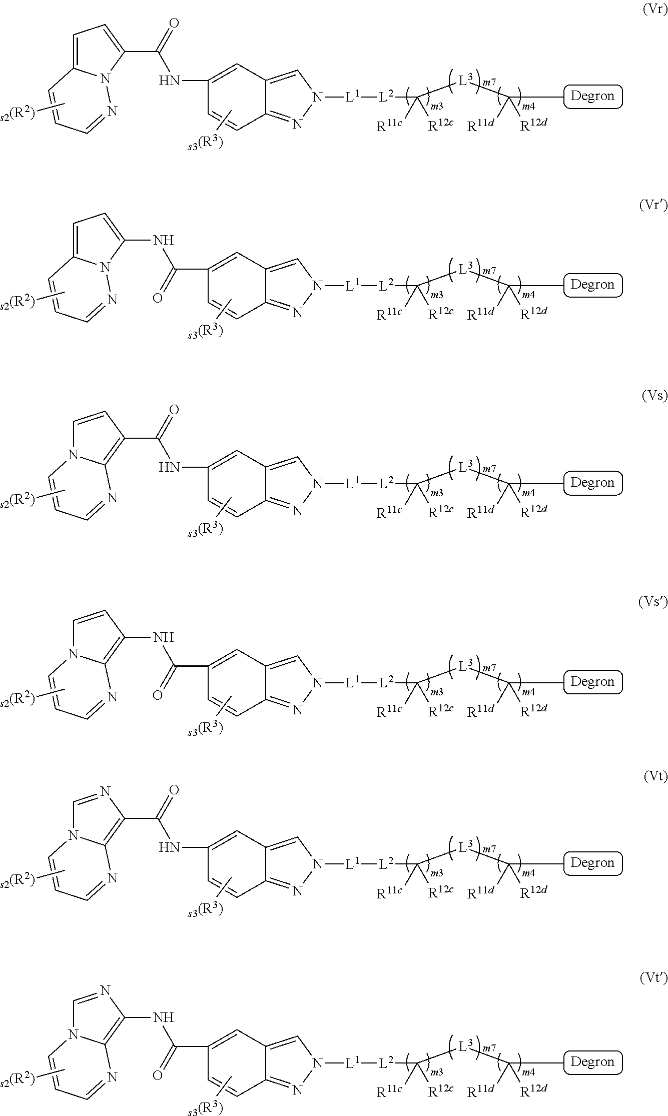

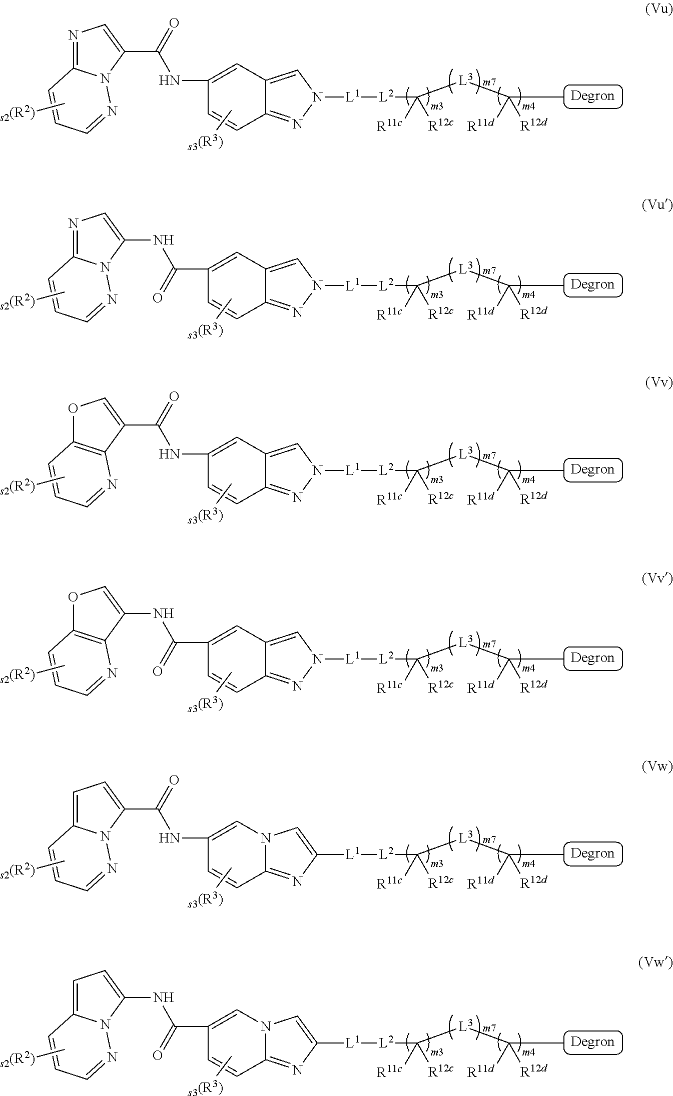

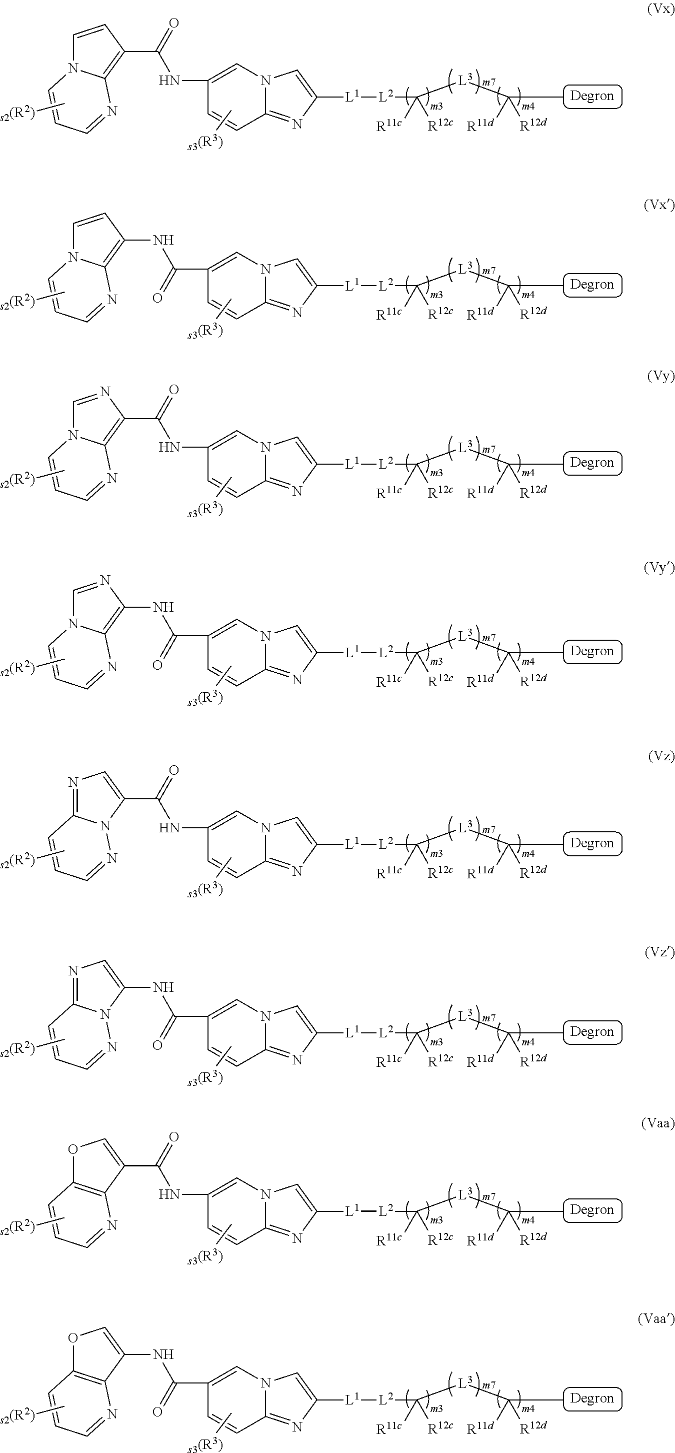

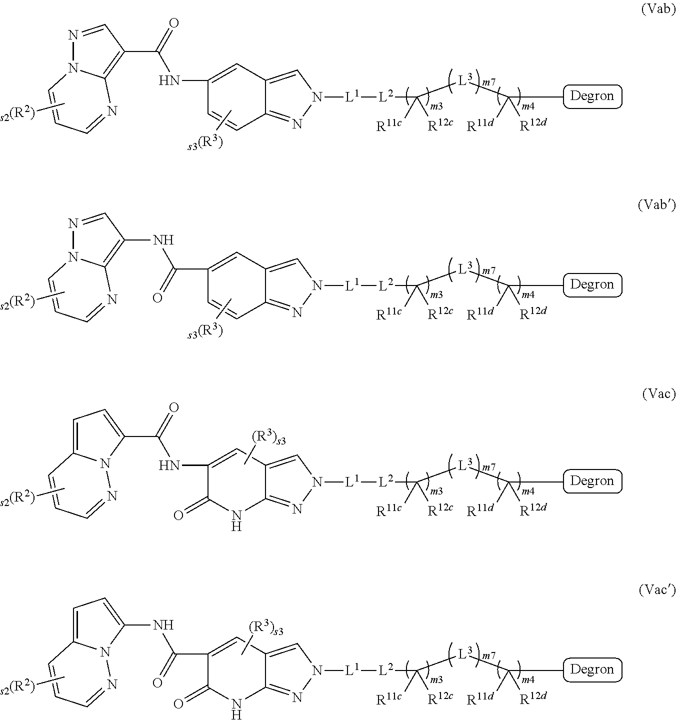

wherein, Y⁴, Y⁷, Y⁸, Y⁹, Y¹⁰, Y¹¹, Y¹², Y¹³, Y¹⁴, Y¹⁵, Y¹⁶ and Y¹⁷ are each independently selected from C or N; Y⁵ and Y⁶ are each independently selected from C, N, O or S; provided that

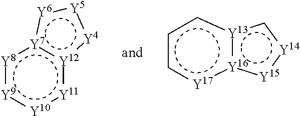

are aromatic ring;
wherein, R², R³, R^{11a}, R^{11b}, R^{11c}, R^{11d}, R^{12a}, R^{12b}, R^{12c}, R^{12d}, L¹, L², L³, s2, s3, m1, m2, m3, m4, m5, m6, m7 and Degron are as defined as Aspect 1.

Aspect 6. The compound of Aspect 1, wherein the compound is selected from formula (VIa) or (Via′)

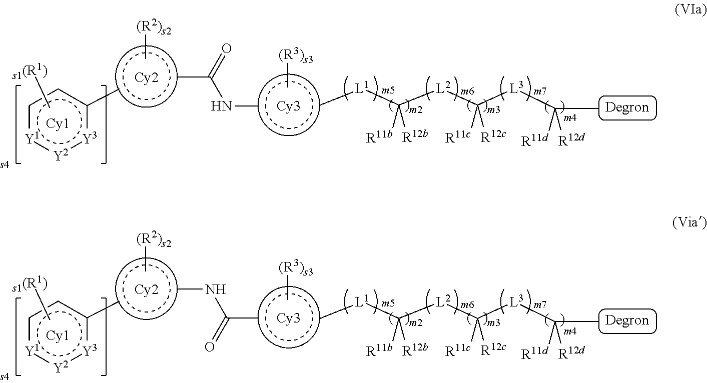

preferably, the compound is selected from (VIb) or (VIb),

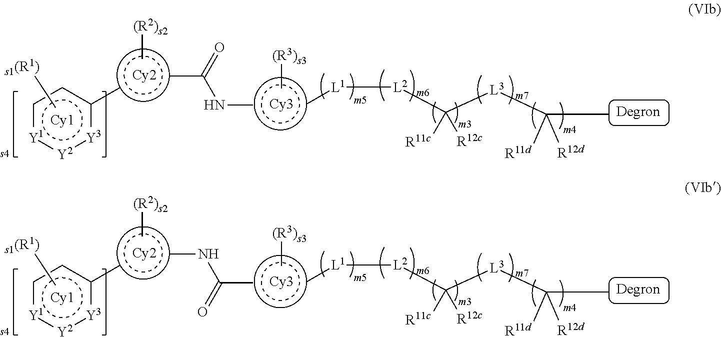

wherein, Y¹, Y², Y³, Cy1, Cy2, Cy3, R¹, R², R³, R^{11b}, R^{11c}, R^{11d}, R^{12b}, R^{12c}, R^{12d}, L¹, L², L³, s1, s2, s3, s4, m2, m3, m4, m5, m6, m7 and Degron are as defined as Aspect 1.

Aspect 7. The compound of anyone of the preceding Aspects, wherein at least one of Y¹, Y² and Y³ is N; preferably, Y¹ is N, Y² and Y³ are C; or Y² is N, Y¹ and Y³ are C; or Y³ is N, Y¹ and Y² are C.

Aspect 8. The compound of anyone of the preceding Aspects, wherein Cy1 is pyridinyl or pyridinonyl.

Aspect 9. The compound of anyone of the preceding Aspects, wherein

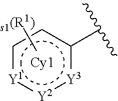

moiety is

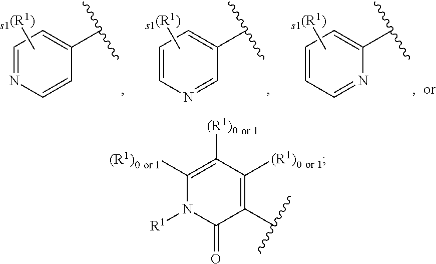

preferably,

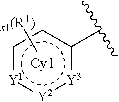

moiety is

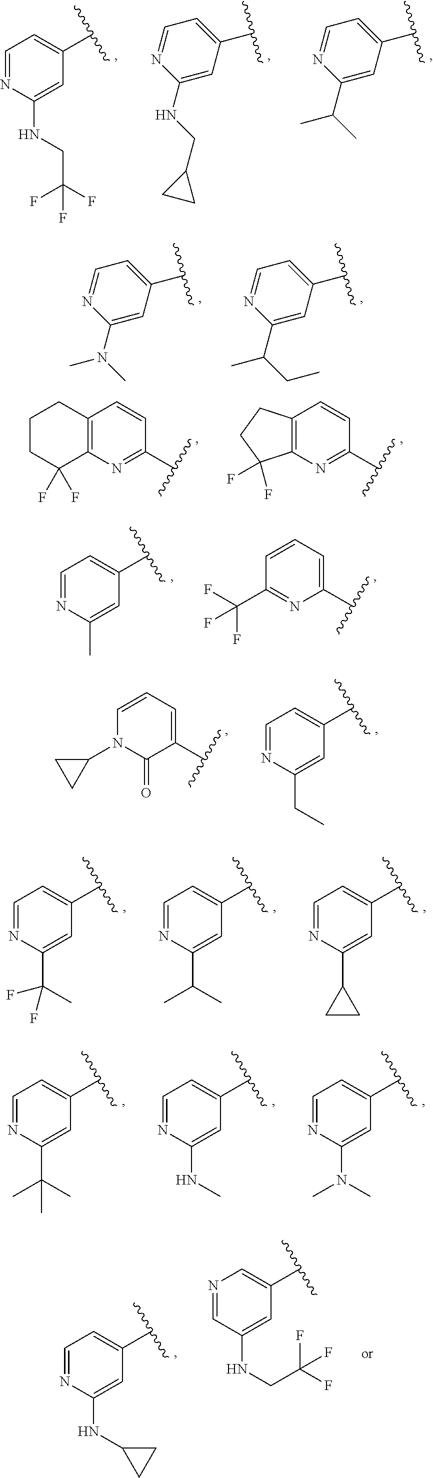

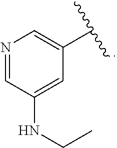 [SMILES: *c1cncc(NCC)c1]

Aspect 10. The compound of anyone of the preceding Aspects, wherein the

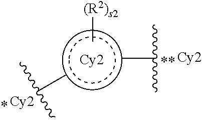

moiety is

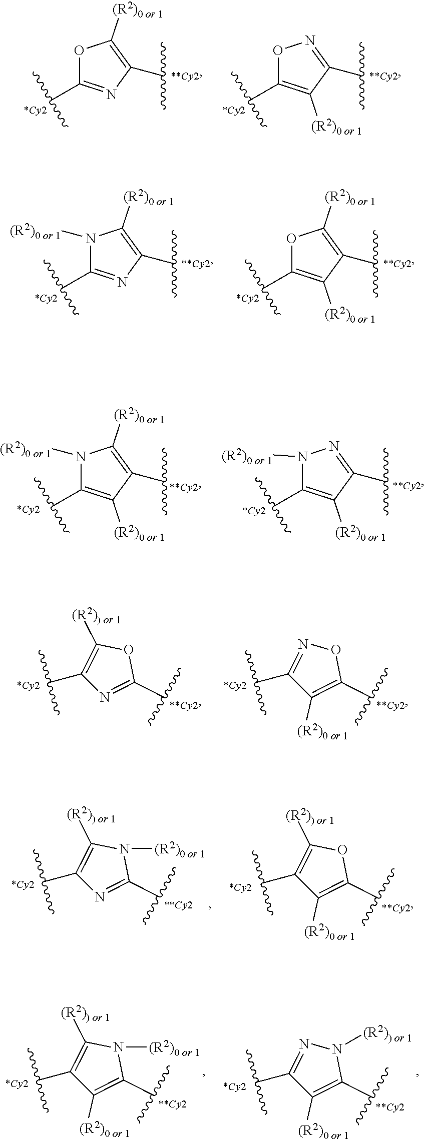

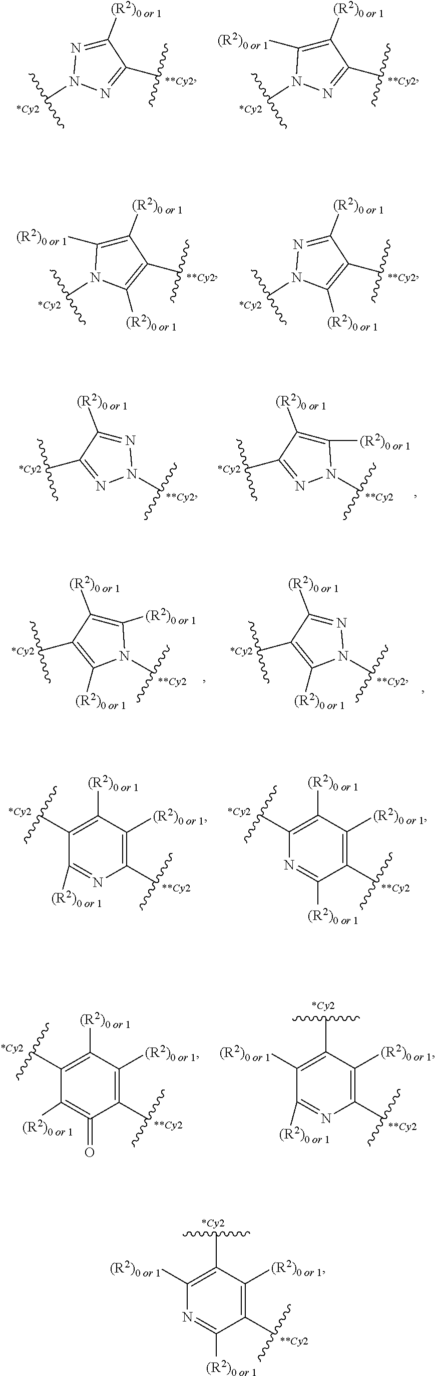

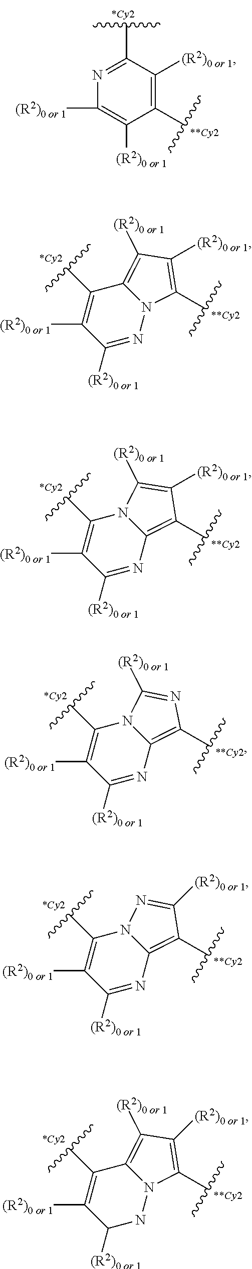

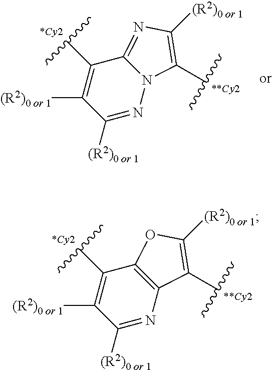

wherein, *^{Cy2} refers to the position attached to the

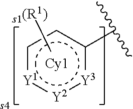

moiety, and **^{Cy2} refers to the position attached to the

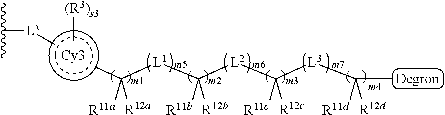

moiety;
preferably,

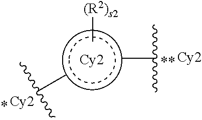

moiety is

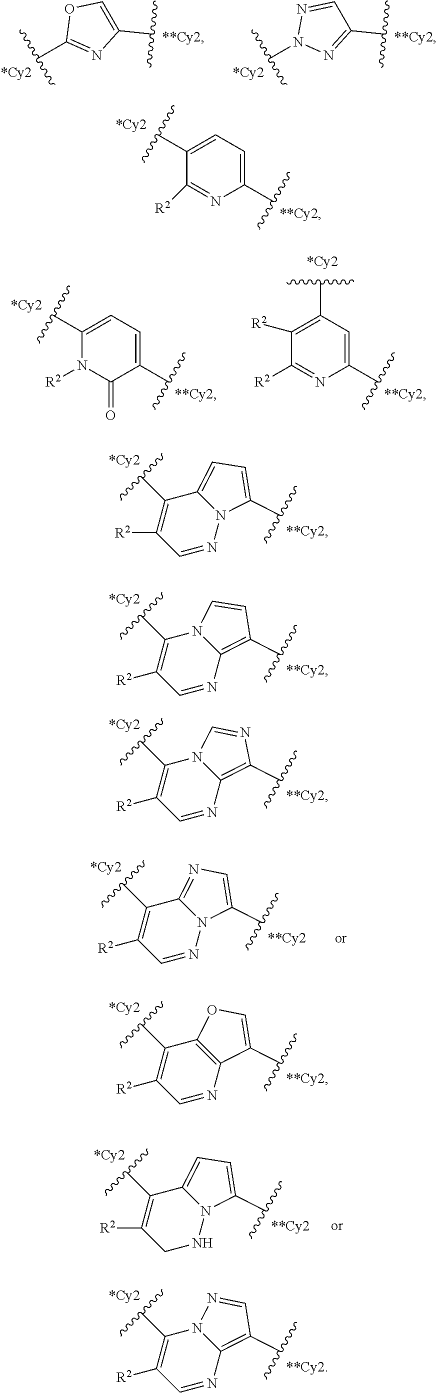

Aspect 11. The compound of anyone of the preceding Aspects, wherein Cy3 is a 5-, 6-, 7-, 8-, 9- or 10-membered aromatic ring; said ring comprising 0, 1, 2 or 3 heteroatoms independently selected from nitrogen, oxygen or optionally oxidized sulfur; said ring is substituted with at least one substituent R³;

In one embodiment, Cy3 is a 5-, 6-, 7-, 8-, 9- or 10-membered aromatic ring, wherein, each of 5-, 6-membered aromatic ring is monocyclic rings, each of 8-, 9- or 10-membered aromatic ring is bicyclic rings, 7-membered aromatic ring is monocyclic or bicyclic rings.

Preferably, Cy3 is a 5- or 9-membered aromatic ring; said ring comprising 0, 1, 2 or 3 heteroatoms independently selected from nitrogen; said ring is substituted with at least one substituent R³;
even more preferably,

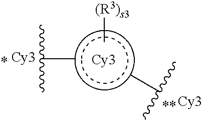

is

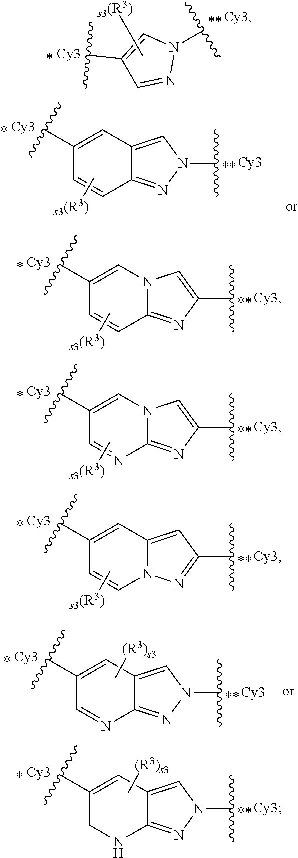

wherein, *^{Cy3} refers to the position attached to the

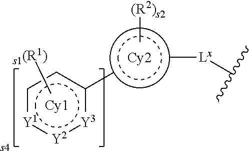

moiety, and **^{Cy3} refers to the position attached to the

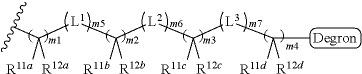

moiety;
even more preferably,

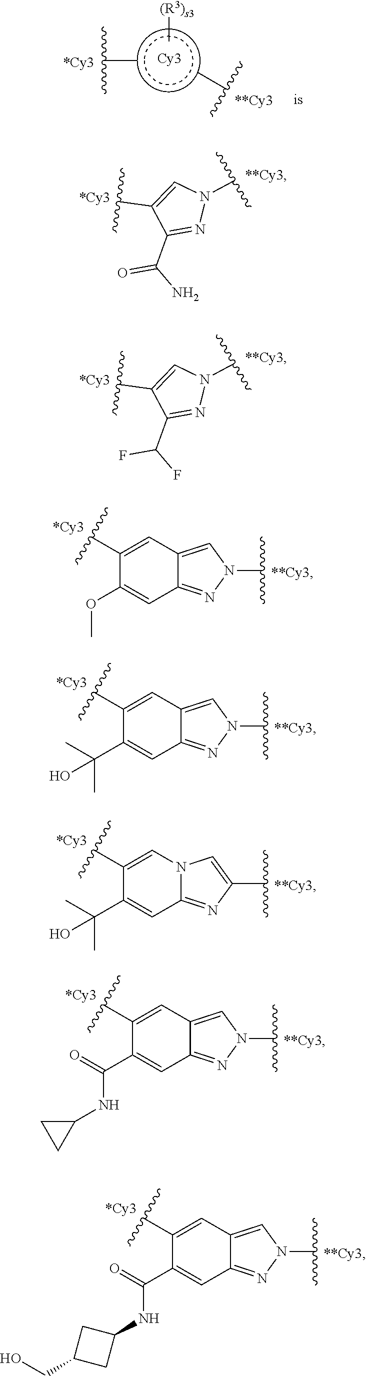

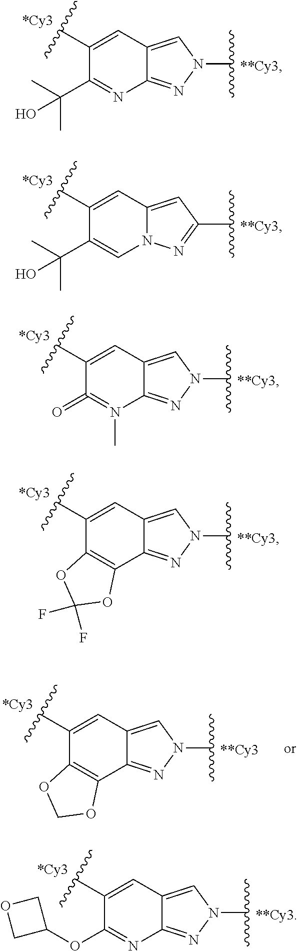

Aspect 12. The compound of any one of the preceding Aspects, wherein R¹, R², and R³ are each independently hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo(═O), —CN, —OR^{1a}, —C(O)R^{1a}, —CO₂R^{1a}, —C(O)NR^{1a}R^{1b}, —NR^{1a}R^{1b}, —NR^{1a}COR^{1b} or —NR^{1a}CO₂R^{1b}; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{1c},
R^{1a} and R^{1b} are each independently selected from hydrogen, methyl, ethyl, propyl, butyl, pentyl, hexyl, heptyl, octyl, —C₁-C₈haloalkyl (preferably —CH₂F, —CHF₂, —CF₃, —CH₂CH₂F, —CH₂CHF₂, —CH₂CF₃), —C₂-C₈alkenyl, —C₂-C₈alkynyl, C₁-C₈alkoxy-C₁-C₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl; each of methyl, ethyl, propyl, butyl, pentyl, hexyl, heptyl, octyl, —C₁-C₈haloalkyl (preferably —CH₂F, —CHF₂, —CF₃, —CH₂CH₂F, —CH₂CHF₂, —CH₂CF₃), —C₂-C₈alkenyl, —C₂-C₈alkynyl, C₁-C₈alkoxy-C₁-C₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent selected from —F, —Cl, —Br, —I, —OH, —CN, oxo (═O), methyl, ethyl, propyl, butyl, pentyl, hexyl, heptyl, octyl, —CH₂OH, —C₂H₄OH, —C₃H₆OH, —C₄H₈OH, —C₅H₁₀OH, —C₆H₁₂OH, —C₇H₁₄OH, —C₈H₁₆OH, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
R^{1c}, at each occurrence, is independently —F, —Cl, —Br, —I, —OH, —CN, oxo (═O), methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 13. The compound of anyone of the preceding Aspects, wherein R¹, R², and R³ are each independently hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, oxo(═O), —CN, —OR^{1a} or —C(O)NR^{1a}R^{1b}, —NR^{1a}R^{1b}; each of methyl, ethyl, propyl, butyl, pentyl, hexyl, heptyl, octyl, cyclopropyl, cyclobutyl, cyclopentyl, cyclohexyl, cycloheptyl, cyclooctyl or 3- to 8-membered heterocyclyl is optionally substituted with at least one substituent R^{1c},
R^{1a} and R^{1b} are each independently selected from hydrogen, methyl, ethyl, propyl, butyl, pentyl, hexyl, heptyl, octyl, —CH₂F, —CHF₂, —CF₃, —CH₂CH₂F, —CH₂CHF₂, —CH₂CF₃, cyclopropyl, cyclobutyl, cyclopentyl, cyclohexyl, cycloheptyl or cyclooctyl; each of methyl, ethyl, propyl, butyl, pentyl, hexyl, heptyl, octyl, —CH₂OH, —C₂H₄OH, —C₃H₆OH, —C₄H₈OH, —C₅H₁₀OH, —C₆H₁₂OH, —C₇H₁₄OH, —C₈H₁₆OH, —CH₂F, —CHF₂, —CF₃, —CH₂CH₂F, —CH₂CHF₂, —CH₂CF₃, cyclopropyl, cyclobutyl, cyclopentyl, cyclohexyl, cycloheptyl or cyclooctyl is optionally substituted with at least one substituent selected from —F, —Cl, —Br, —I, —OH, —CN, oxo (═O), methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
R^{1c}, at each occurrence, is independently —F, —Cl, —Br, —I, —OH;
preferably, R¹, R², and R³ are each independently H, —F, —Cl, —Br, —I, —CH₃, —CH₂CH₃, —CF₃, —CF₂CH₃, —CH₂CF₃, —CH(CH₃)₂, —C(CH₃)₃, —NHCH₃, —NHCH₂CH₃, —N(CH₃)CH₂CH₃, —N(CH₃)₂, —CHF₂, oxo (═O), —CN, cyclopropyl, —CONH₂, —OMe, —OEt, —OCH₂CH₂CH₃, —OCH(CH₃)₂,

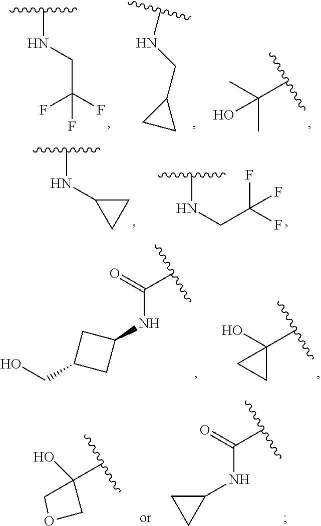

even more preferably, R¹ is H, —CH₃, —CH₂CH₃, —CF₃, —CF₂CH₃, —CH(CH₃)₂, —C(CH₃)₃, —NHCH₃, —NHCH₂CH₃, —N(CH₃)CH₂CH₃, —N(CH₃)₂, oxo (═O), cyclopropyl,

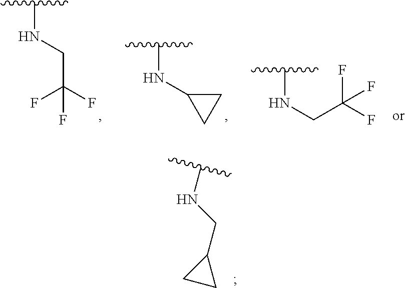

R² is H, —CF₃, cyclopropyl, oxo (═O), —F, —Cl, —Br, —I or —CN;
R³ is H, —CH₃; oxo (═O), —CHF₂, —CONH₂, —OMe, —OCD₃, —OEt, —OCH₂CH₂CH₃, —OCH(CH₃)₂,

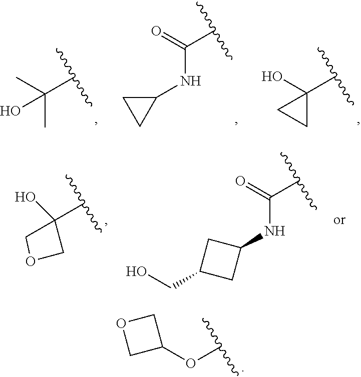

Aspect 14. The compound of any one of the preceding Aspects, wherein two R³ together with the atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent R^{1c};
R^{1c}, at each occurrence, is independently —F, —Cl, —Br, —I, —OH, —CN, oxo (═O), methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.
preferably, two R³ together with the atoms to which they are attached, form a 3-, 4-, 5- or 6-membered ring, said ring comprising 0, 1, 2 or 3 heteroatoms independently selected from oxygen; said ring is optionally substituted with at least one substituent R^{1c};
R^{1c}, at each occurrence, is independently —F, —Cl, —Br, —I, —OH, —CN, oxo (═O), methyl, ethyl, propyl, cyclopropyl, cyclobutyl or cyclopentyl.

Aspect 15. The compound of any one of the preceding Aspects, wherein R^{11a}, R^{11b}, R^{11c}, R^{11d}, R^{12a}, R^{12b}, R^{12c} and R^{12d} are each independently hydrogen, F, Cl, Br, I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptoxy, octyloxy, cyclopropyl, cyclobutyl, cyclopentyl, cyclohexyl, cycloheptyl or cyclooctyl; each of said methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptoxy, octyloxy, cyclopropyl, cyclobutyl, cyclopentyl, cyclohexyl, cycloheptyl or cyclooctyl is optionally substituted with at least one substituent selected from hydrogen, F, Cl, Br, I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptoxy, octyloxy or —CN; or
(R^{11a} and R^{12a}), (R^{11b} and R^{12b}), (R^{11c} and R^{12c}) or (R^{11d} and R^{12d}) together with the carbon atoms to which they are attached, form a 3-, 4-, 5-, 6-, 7- or 8-membered ring, said ring comprising 0, 1, 2, or 3 heteroatoms independently selected from nitrogen, oxygen or sulfur; said ring is optionally substituted with at least one substituent selected from hydrogen, halogen, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptoxy, octyloxy or —CN.

Aspect 16. The compound of any one of the preceding Aspects, wherein

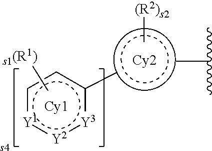

moiety is

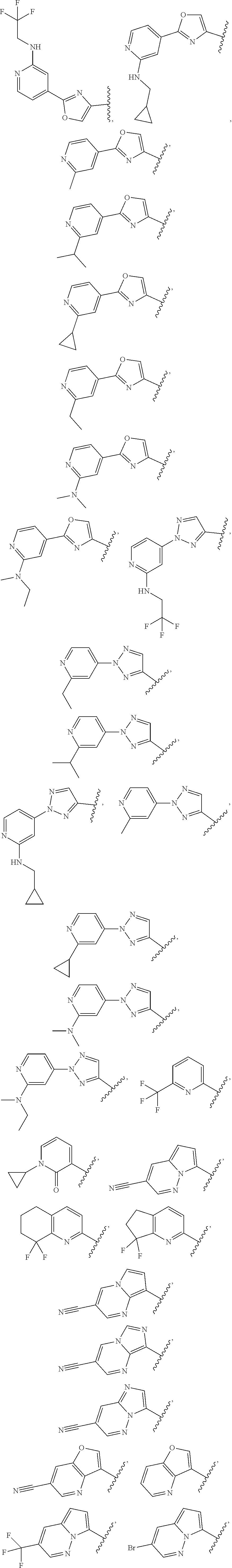

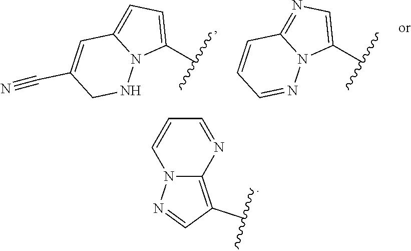

Aspect 17. The compound of any one of the preceding Aspects, wherein the

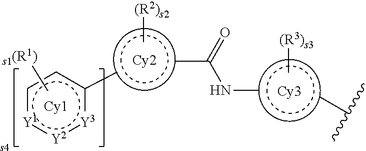

moiety is

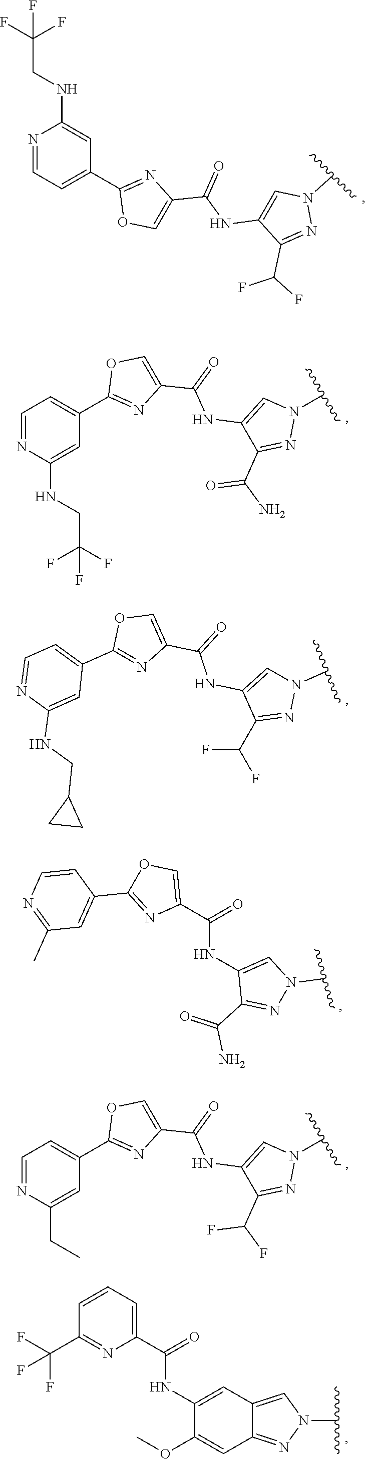

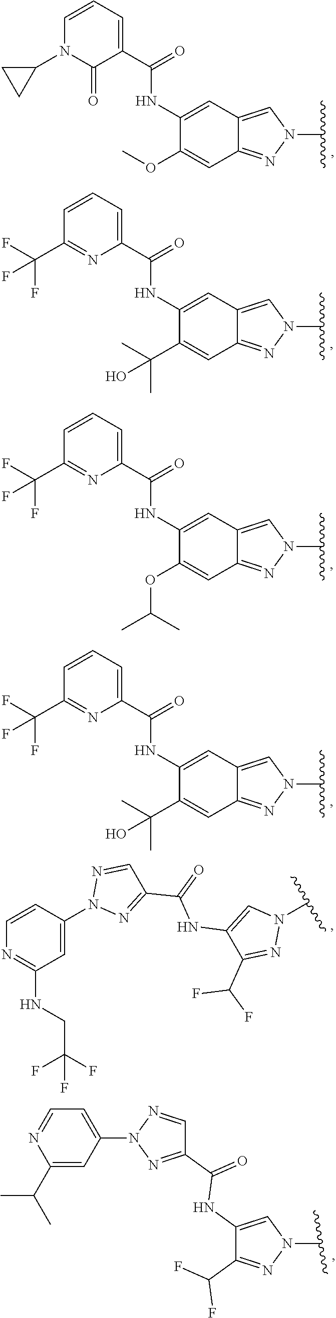

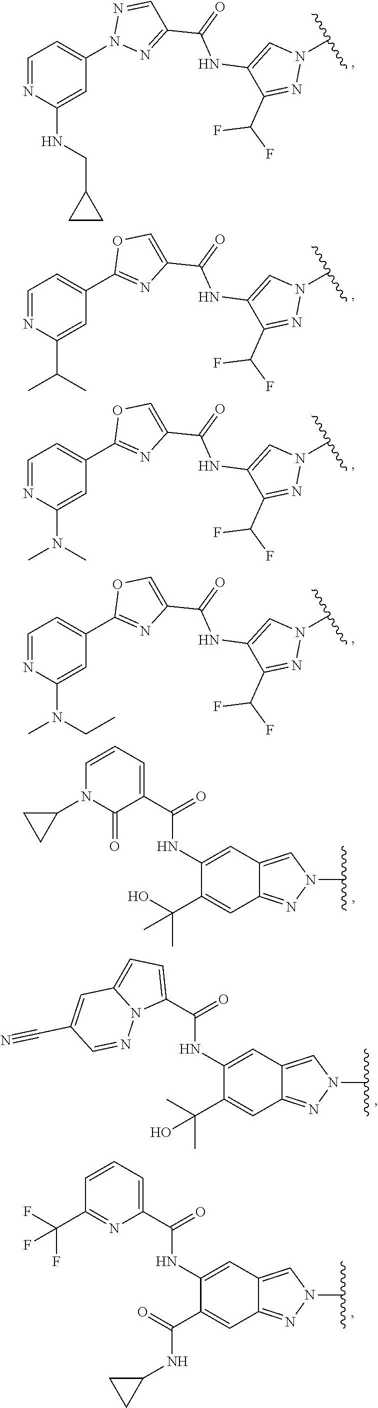

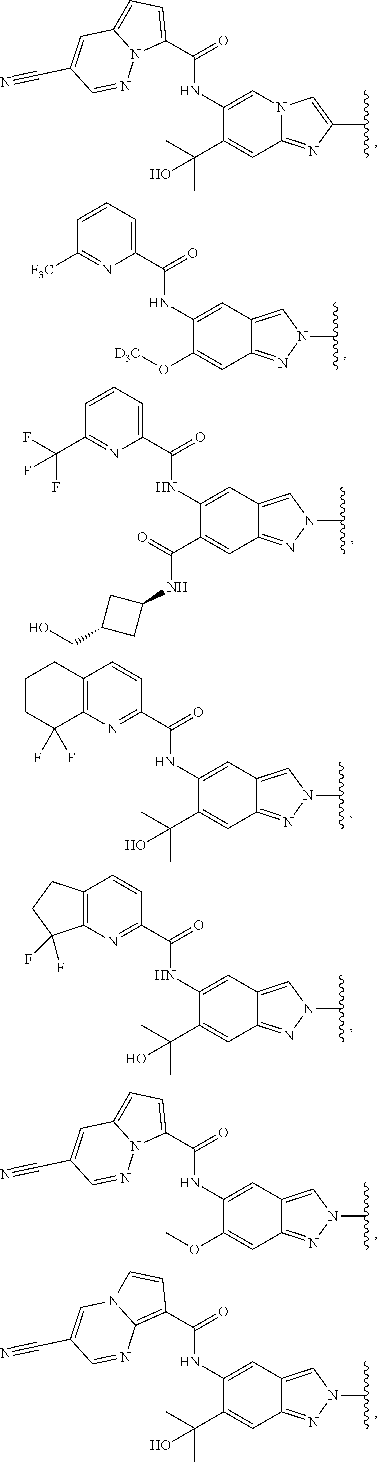

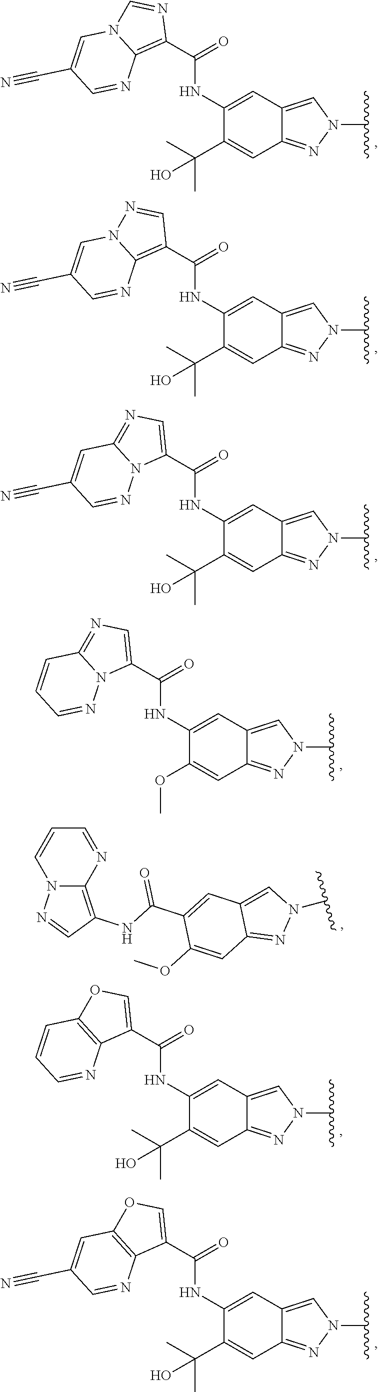

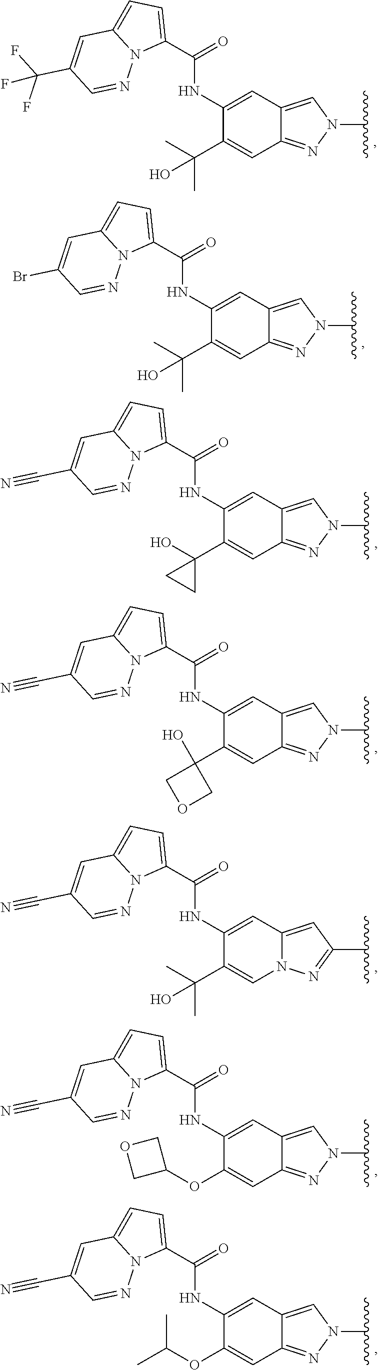

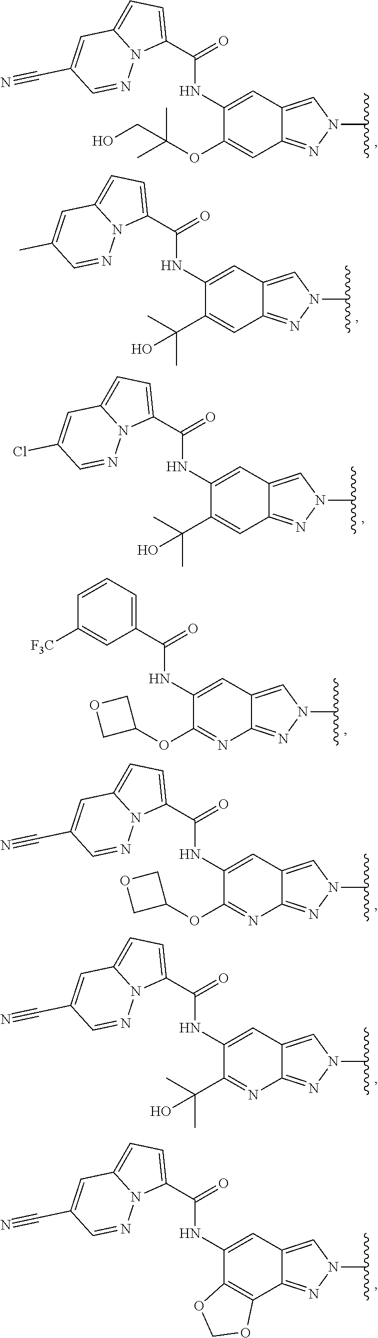

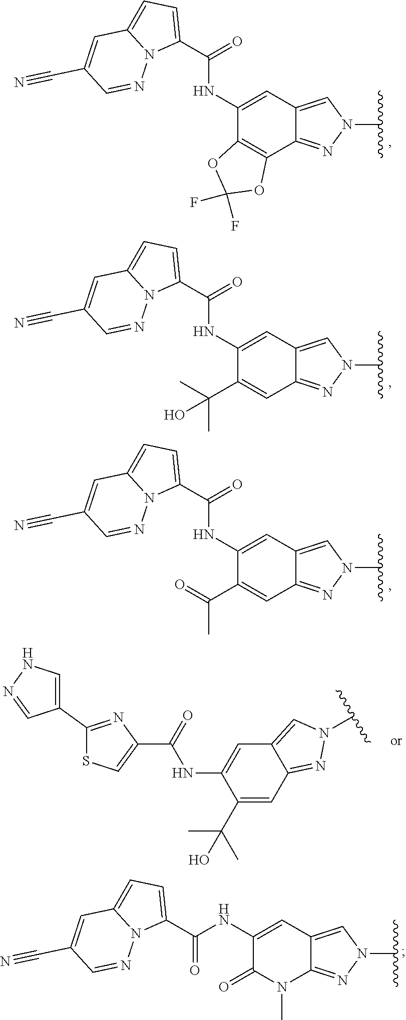

preferably, the

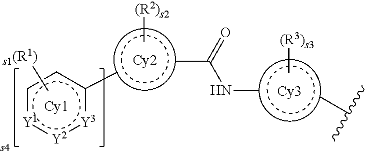

moiety is

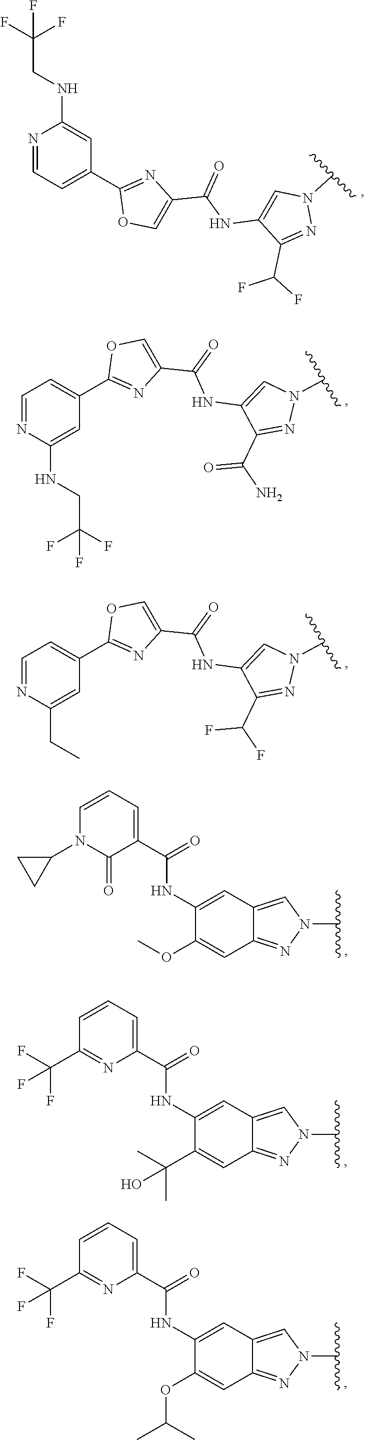

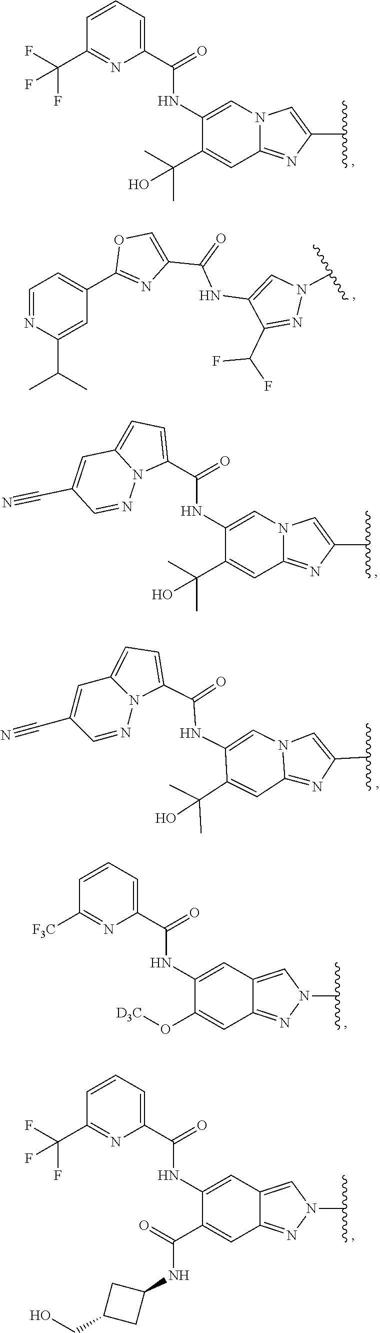

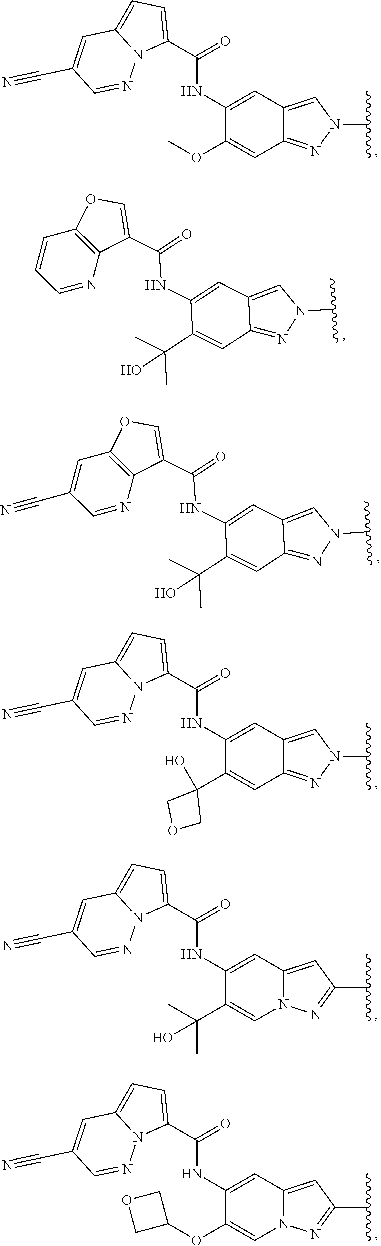

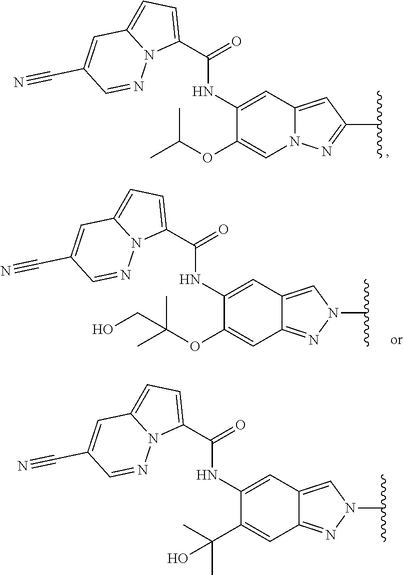

Aspect 18. The compound of any one of the preceding Aspects, wherein R^{11a}, R^{11b}, R^{11c}, R^{11d}, R^{12a}, R^{12b}, R^{12c} and R^{12d} are each independently hydrogen, F, Cl, Br, I, methyl, ethyl, propyl, butyl, pentyl, hexyl, heptyl, octyl, methoxy, ethoxy, propoxy, butoxy, pentoxy, hexoxy, heptoxy, octyloxy, cyclopropyl, cyclobutyl, cyclopentyl, cyclohexyl, cycloheptyl or cyclooctyl; preferably, R^{11a}, R^{11b}, R^{11c}, R^{11d}, R^{12a}, R^{12b}R^{12c} and R^{12d} are each independently hydrogen, F, Cl, Br, I, methyl, ethyl or propyl; more preferably, R^{11a}, R^{11b}, R^{11c}, R^{11d}, R^{12a}, R^{12b}, R^{12c} and R^{12d} are each independently hydrogen or methyl; or
(R^{11a} and R^{12a}), (R^{11b} and R^{12b}), (R^{11c} and R^{12c}) or (R^{11d} and R^{12d}) together with the carbon atoms to which they are attached, form a 3-, 4-, 5-, 6-, 7- or 8-membered ring, said ring comprising 0, 1, 2, or 3 heteroatoms independently selected from nitrogen, oxygen or sulfur; preferably (R^{11a} and R^{12a}), (R^{11b} and R^{12b}), (R^{11c} and R^{12c}) or (R^{11d} and R^{12d}) together with the carbon atoms to which they are attached, form a 3-, 4- or 5-membered cycloalkyl ring.

Aspect 19. The compound of any one of the preceding Aspects, wherein L¹ is selected from —O—, —N(R^{a})—,

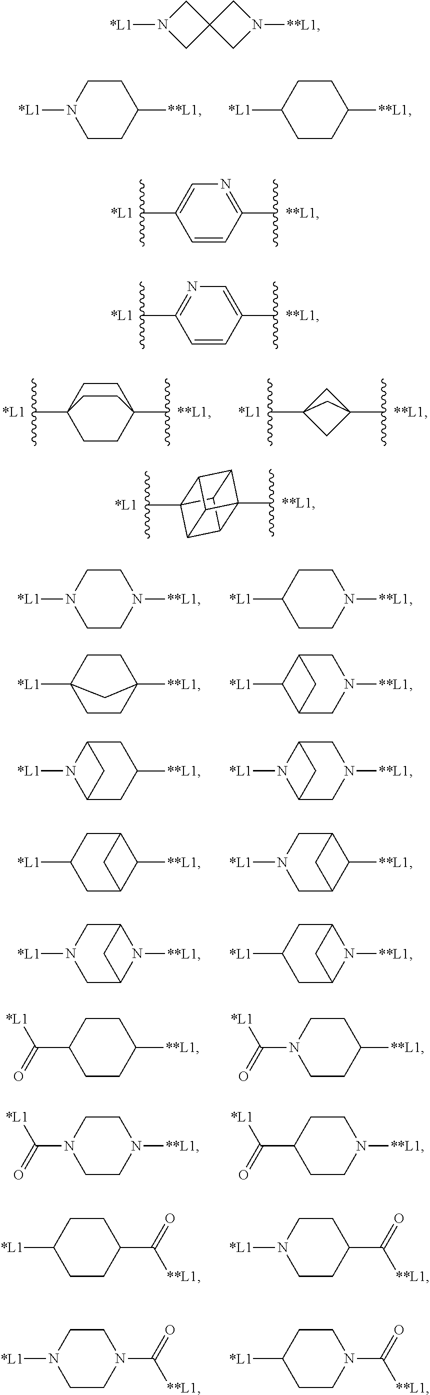

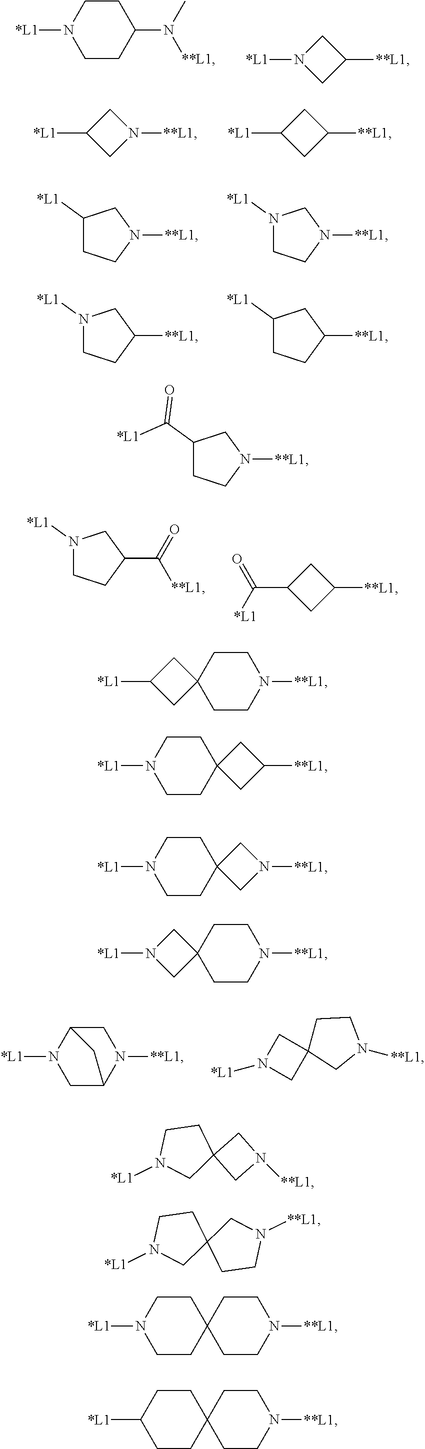

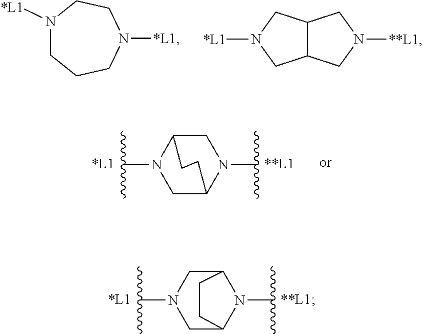

wherein each of said

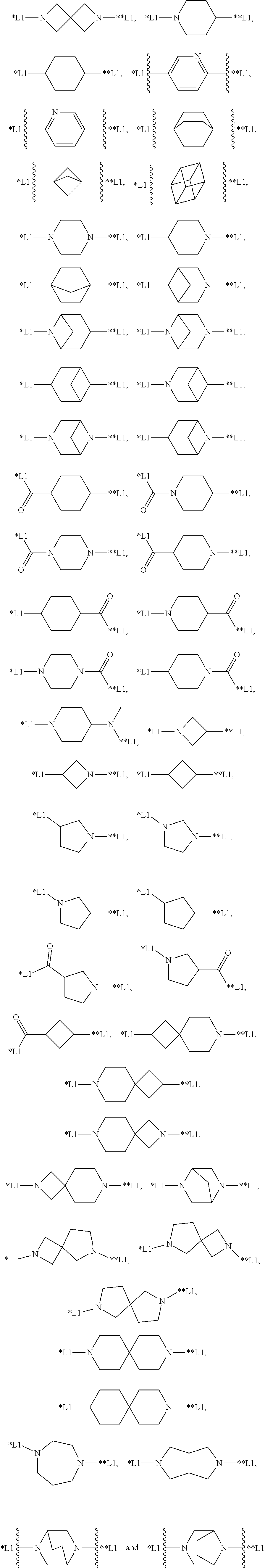

is optionally substituted with at least one R^{L1c};
each of said R^{L1c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L1c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, or octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂—C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 20. The compound of any one of the preceding Aspects, wherein L¹ is selected from —O—, —N(CH₃)—,

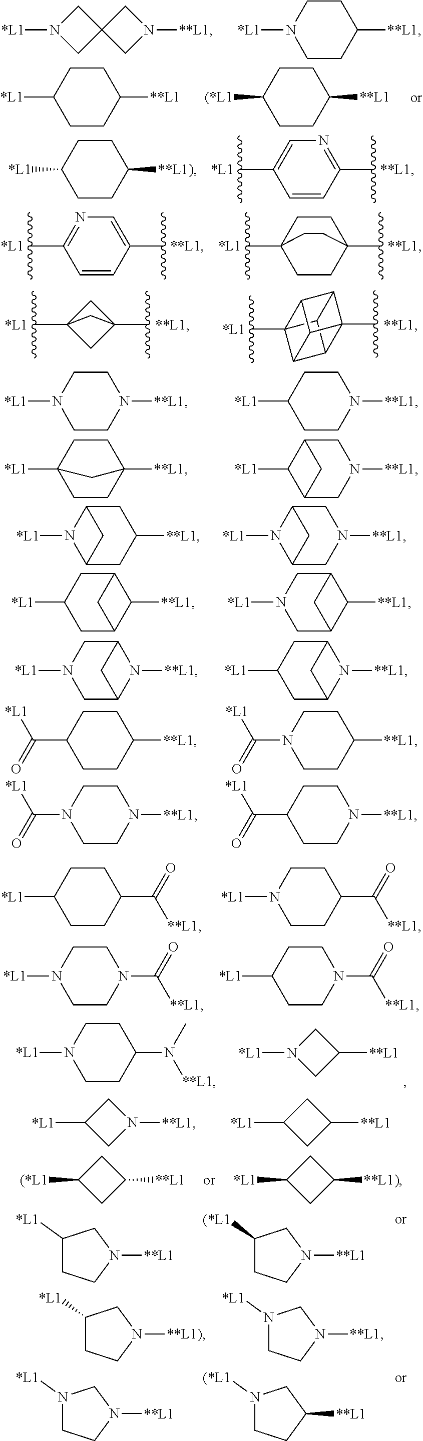

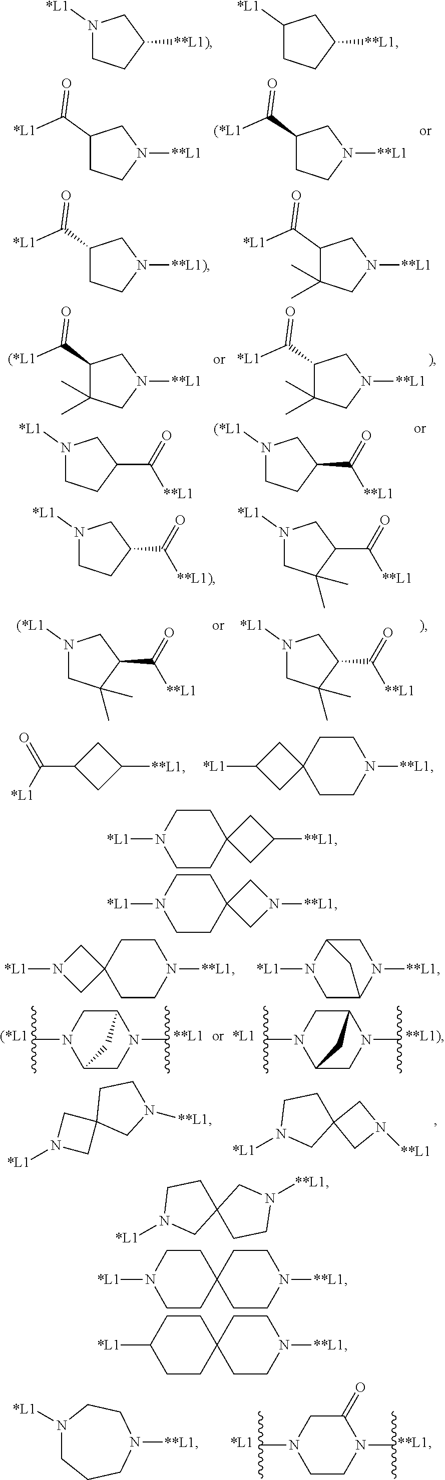

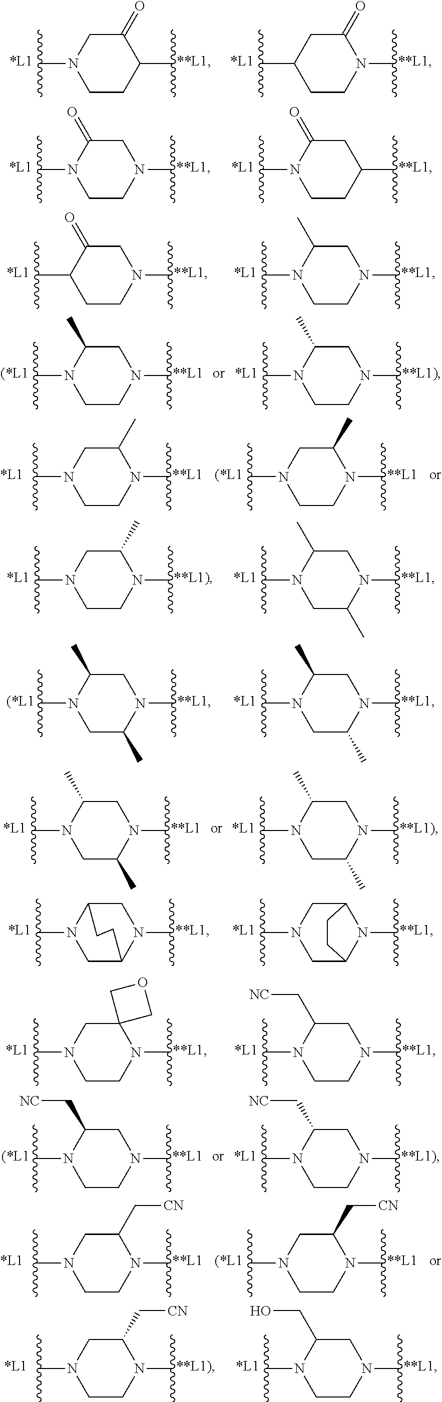

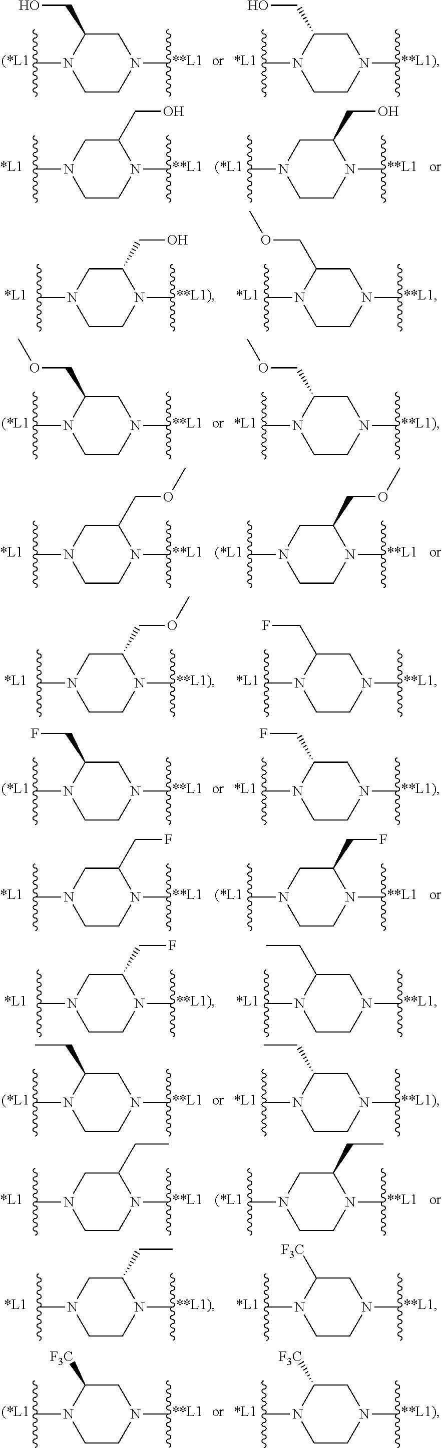

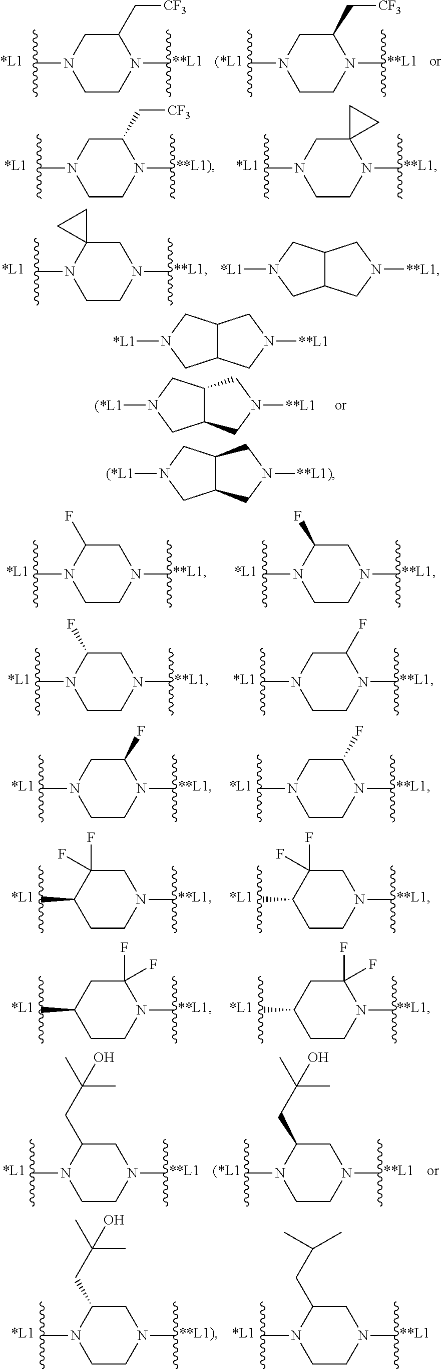

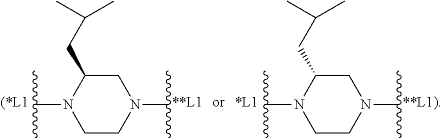 [SMILES: *N1CCN(*)[C@@H](CC(C)C)C1 ; *N1CCN(*)[C@H](CC(C)C)C1]

Aspect 21. The compound of any one of the preceding Aspects, wherein L² is selected from —O—, —N(R^{a})—,

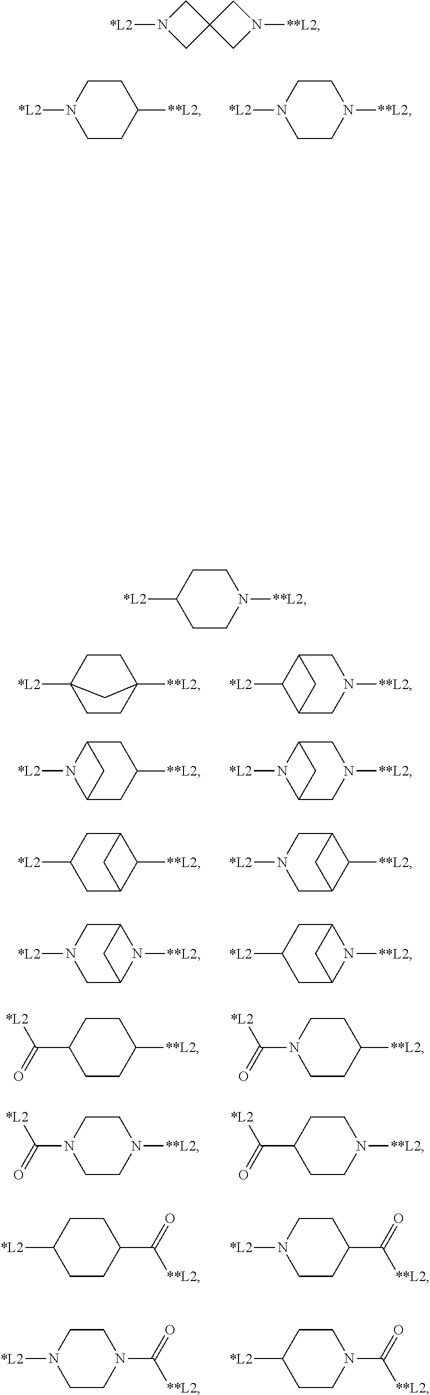

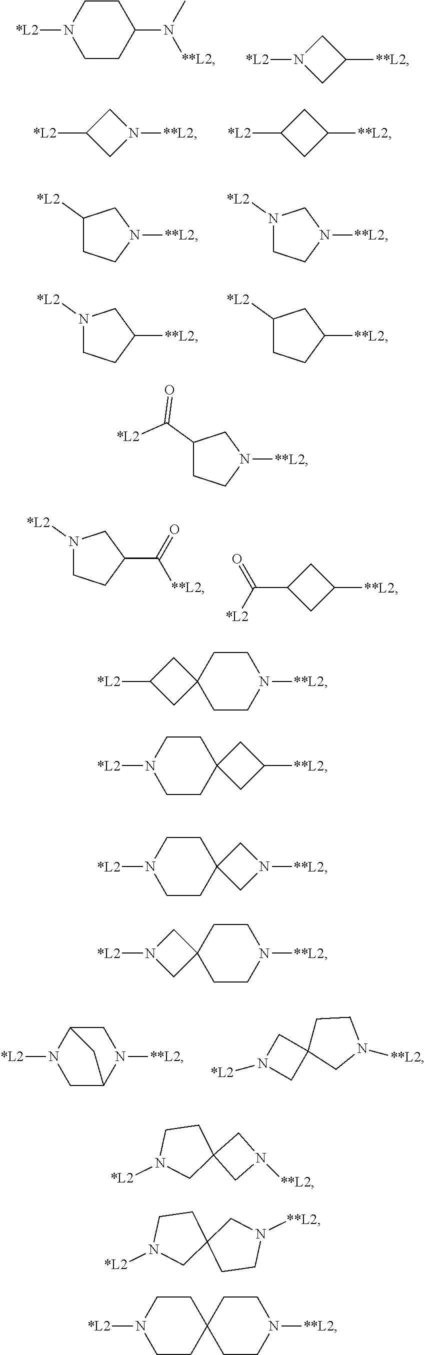

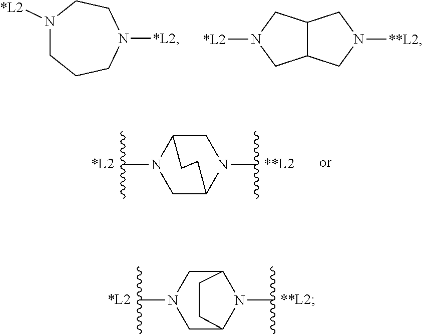

wherein each of said

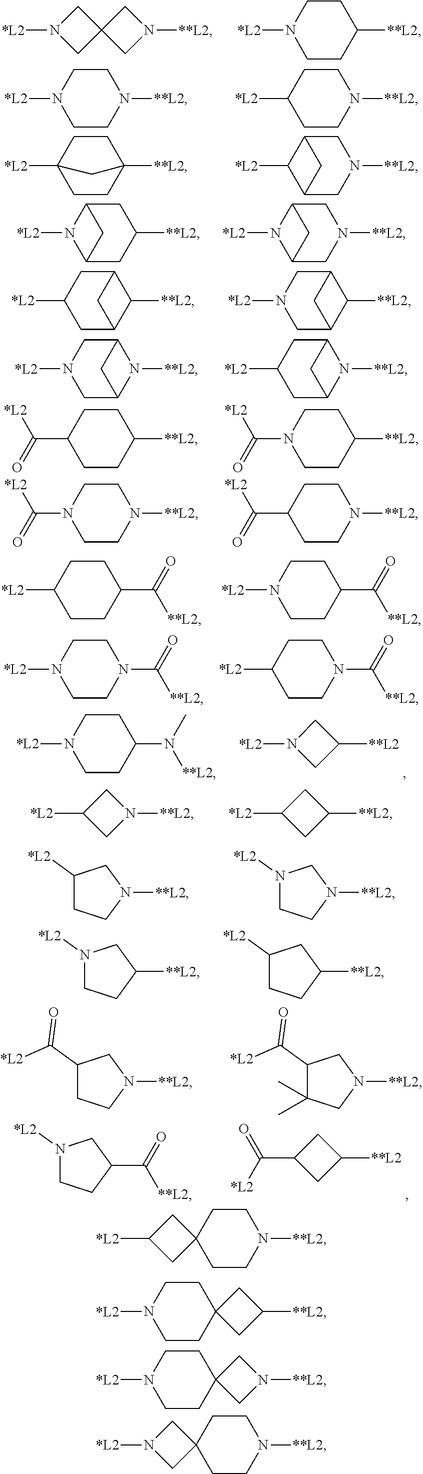

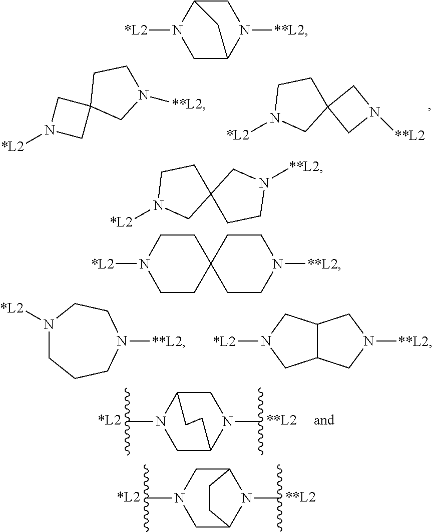

is optionally substituted with at least one R^{L2c};
each of said R^{L2c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca}
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L2c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 22. The compound of any one of the preceding Aspects, wherein L² is selected from —O—, —N(CH₃)—,

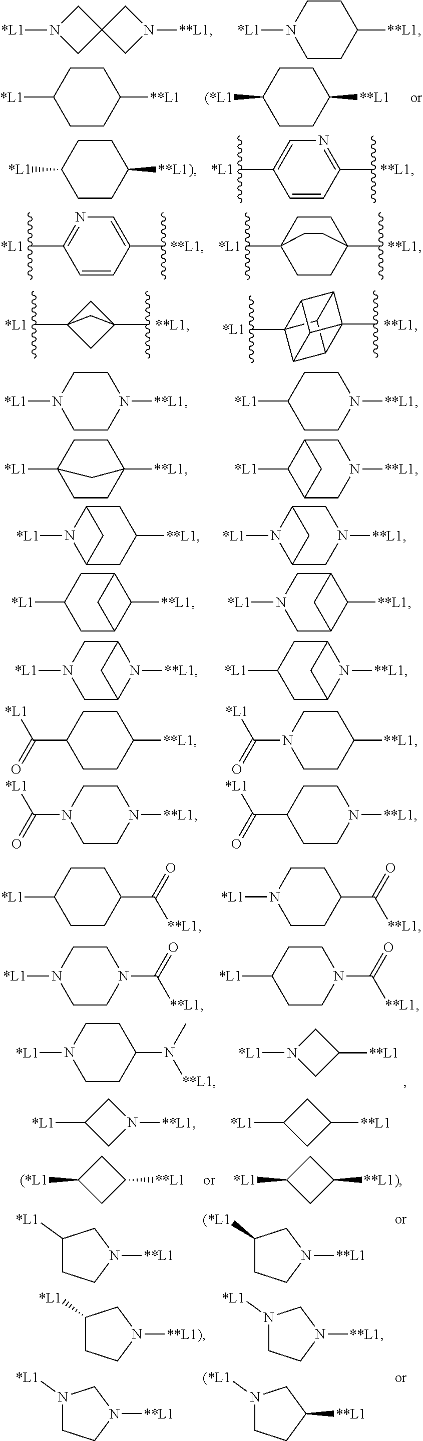

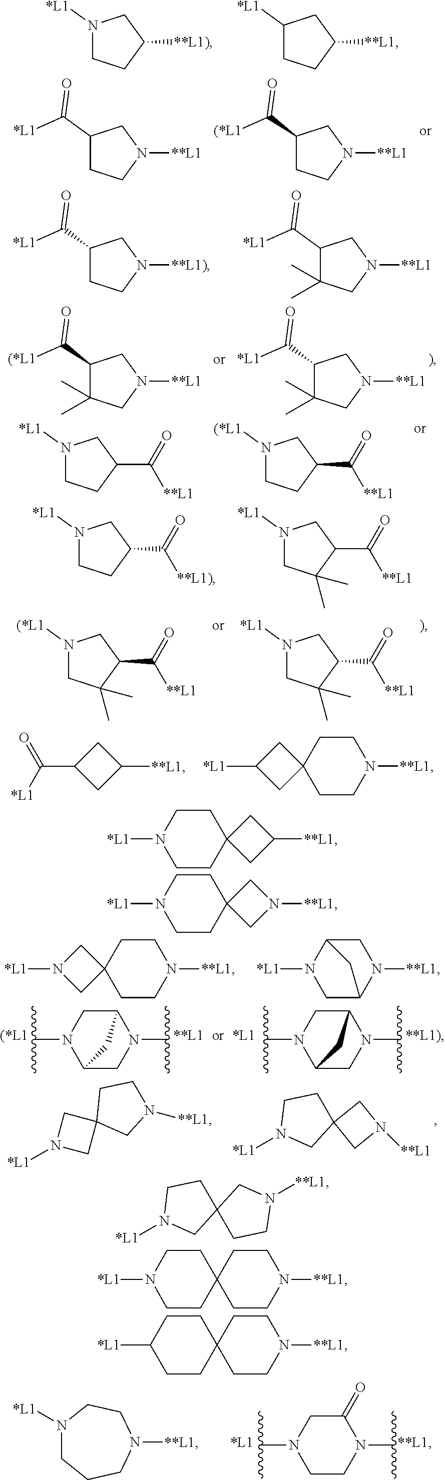

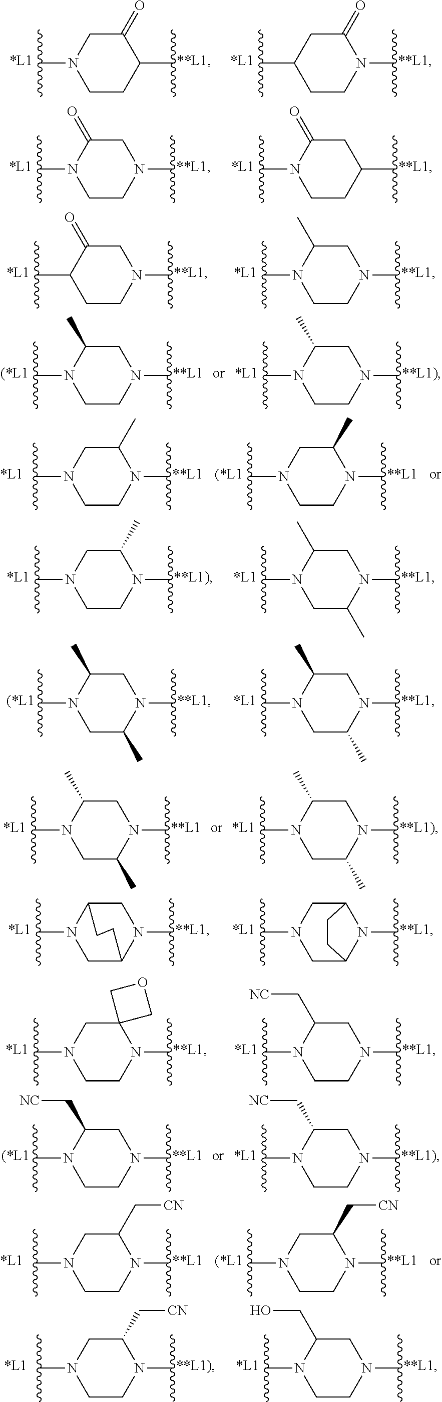

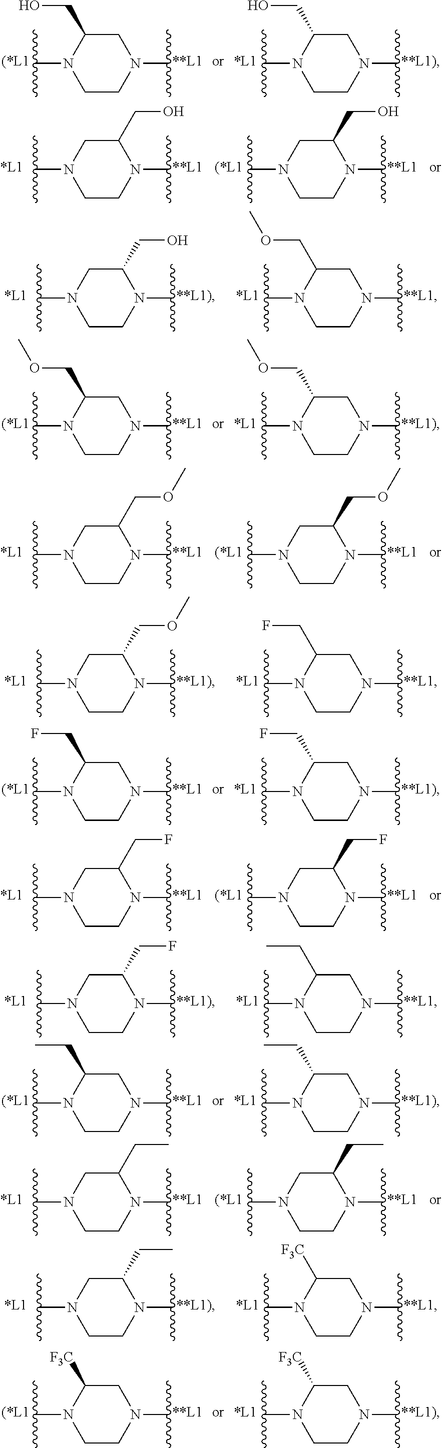

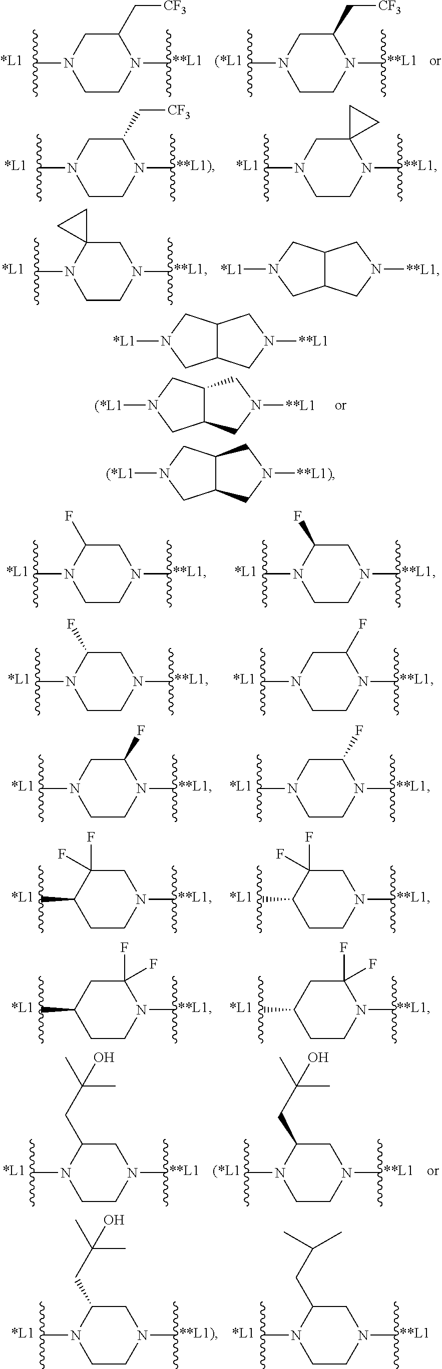

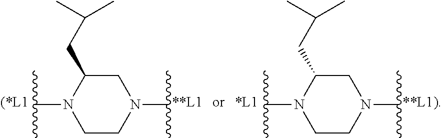 [SMILES: *N1CCN(*)[C@@H](CC(C)C)C1 ; *N1CCN(*)[C@H](CC(C)C)C1]

Aspect 23. The compound of any one of the preceding Aspects, wherein the

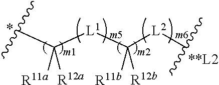

moiety is

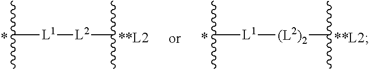

preferably, the

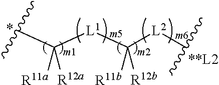

moiety is

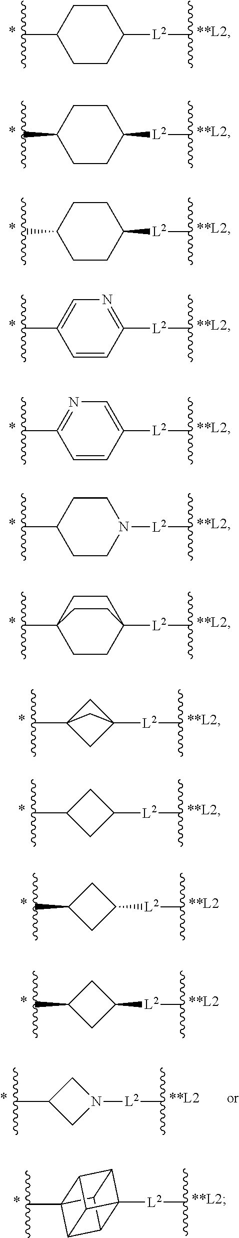

more preferably, the

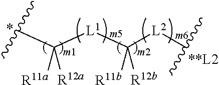

moiety is

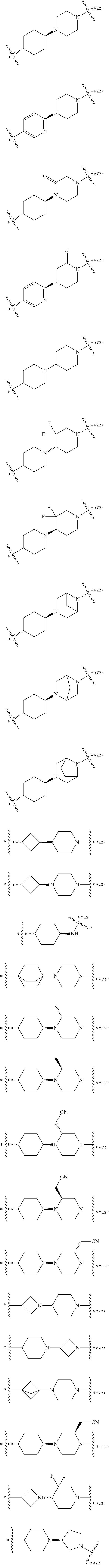

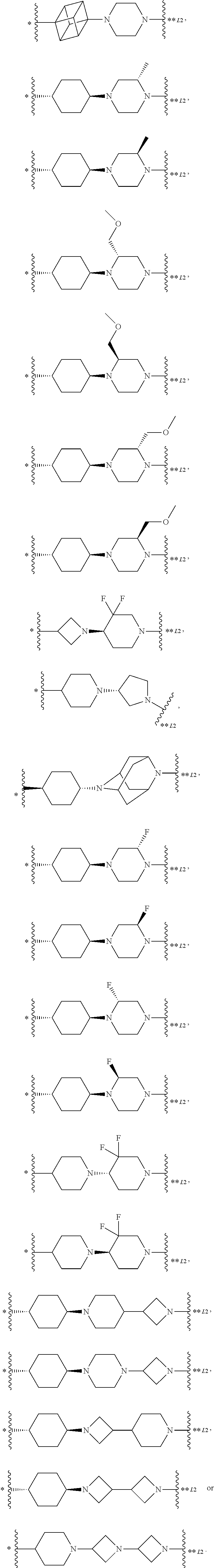

Aspect 24. The compound of any one of the preceding Aspects, wherein L³ is selected from —O—, —N(R^{a})—,

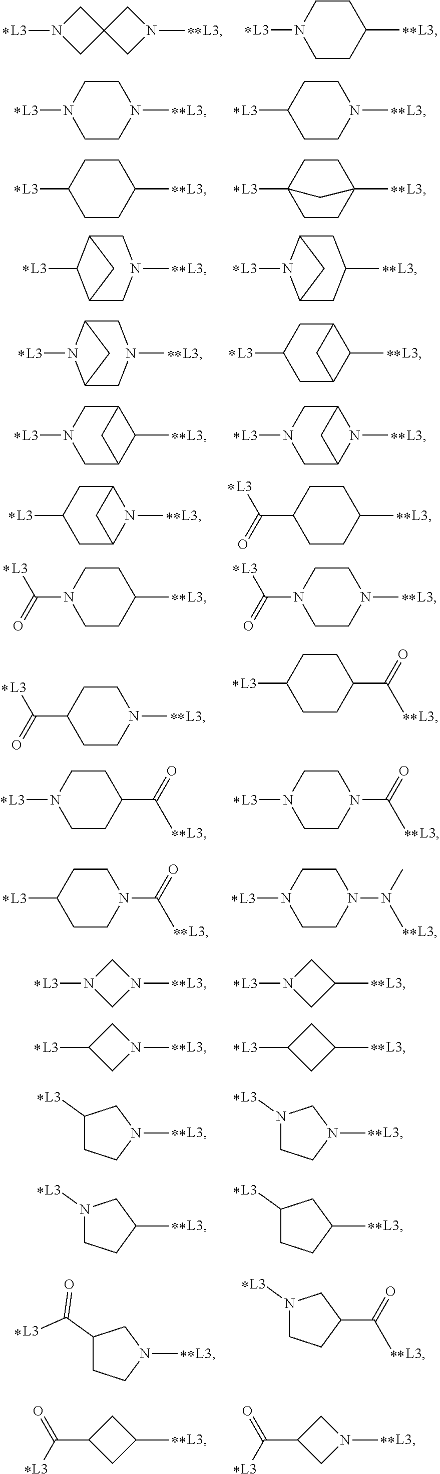

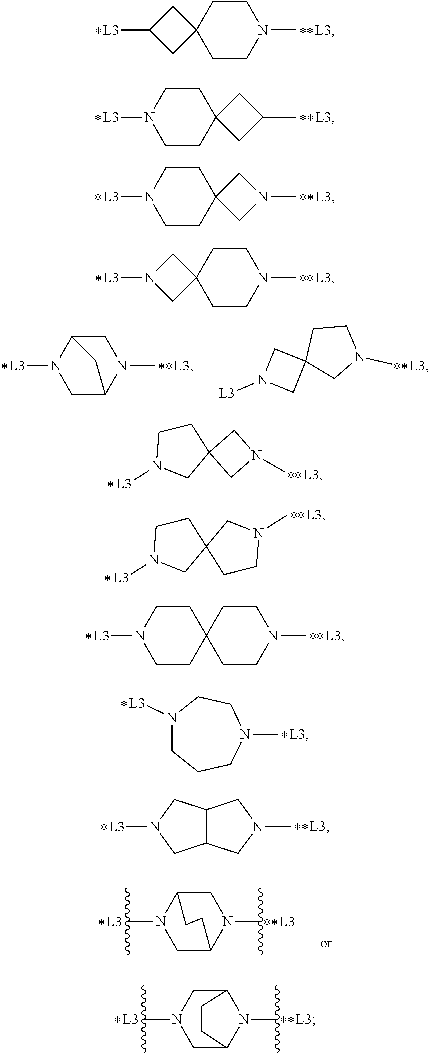

wherein each of said

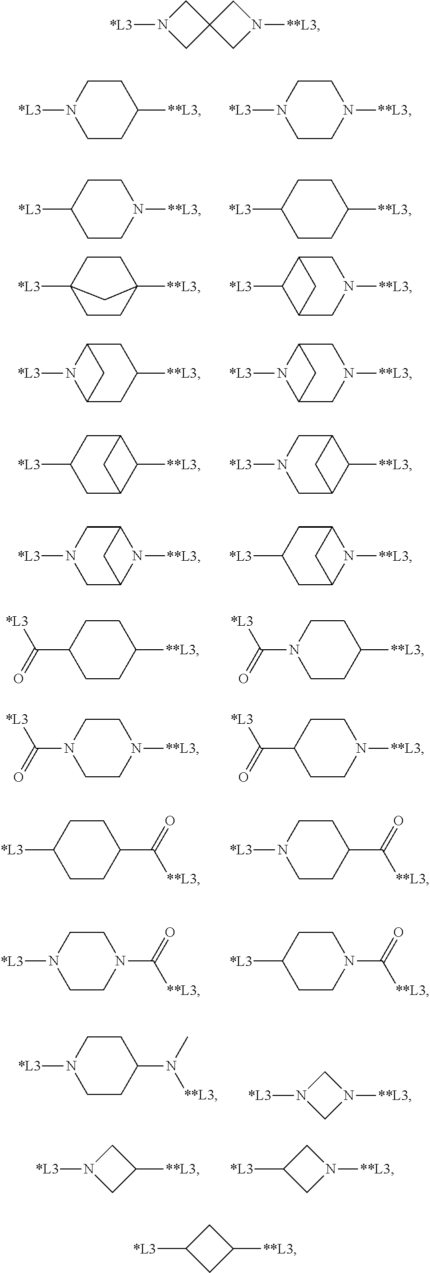

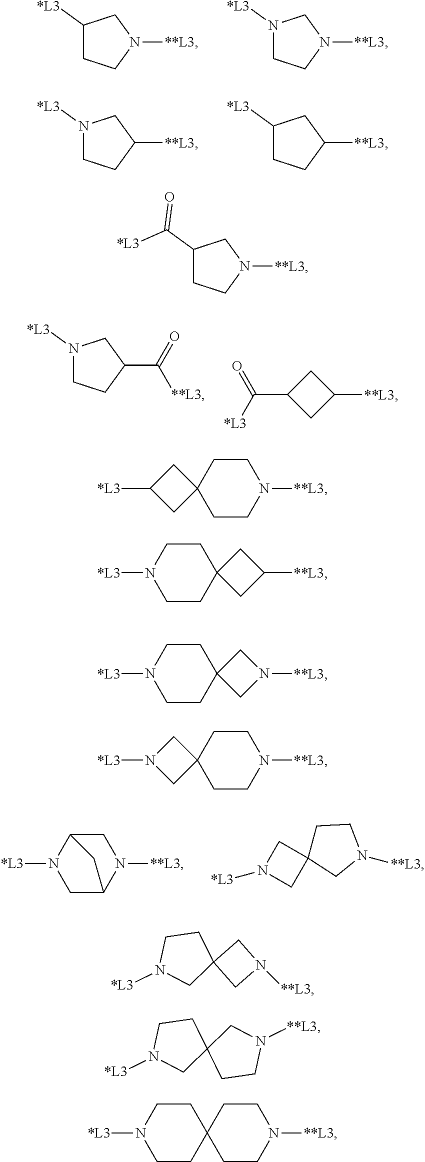

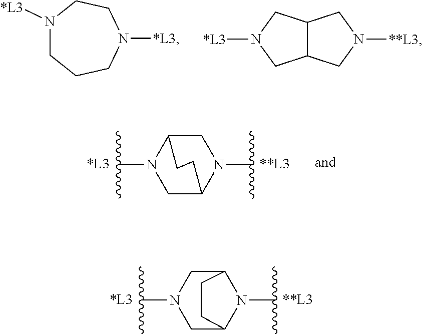

is optionally substituted with at least one R^{L3c};
each of said R^{L3c} is independently oxo (═O) F, Cl, Br, I, hydroxy, methyl, ethyl, propyl butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L3c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 25. The compound of any one of the preceding Aspects, wherein L³ is selected from —O—, —N(CH₃)—, —NH—,

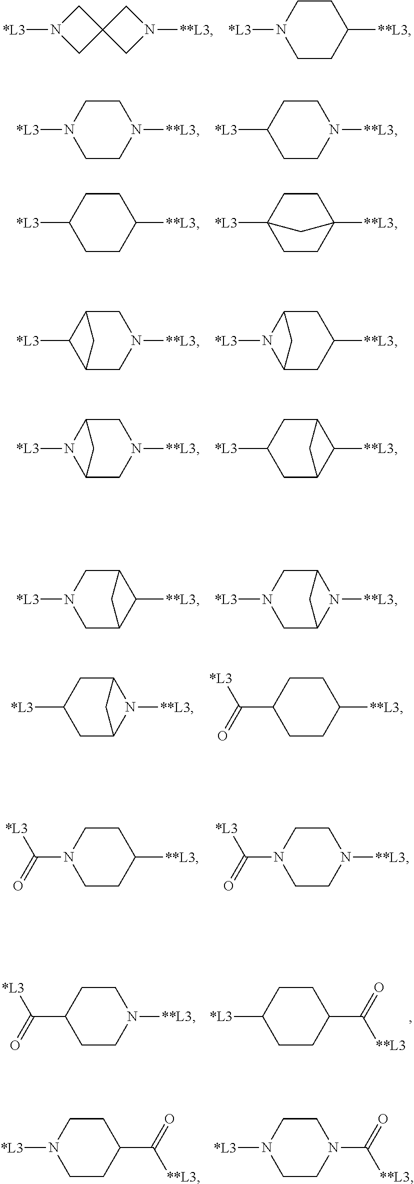

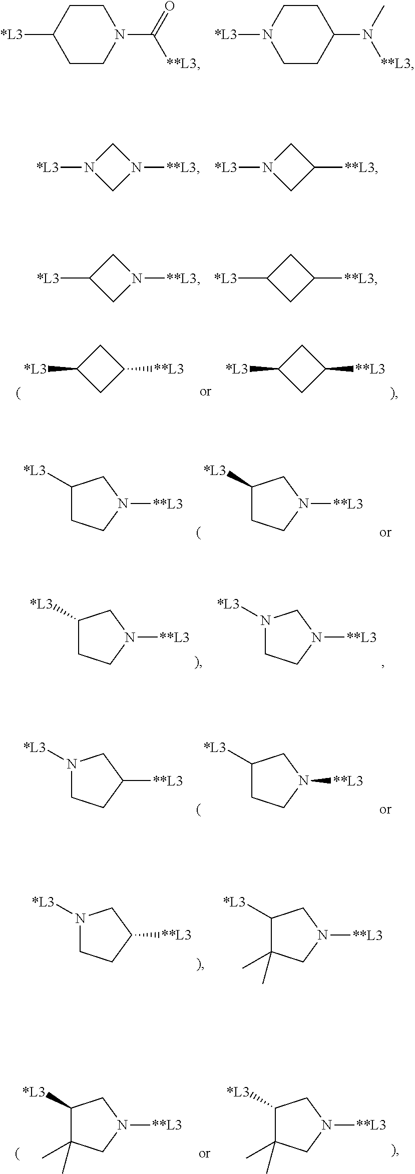

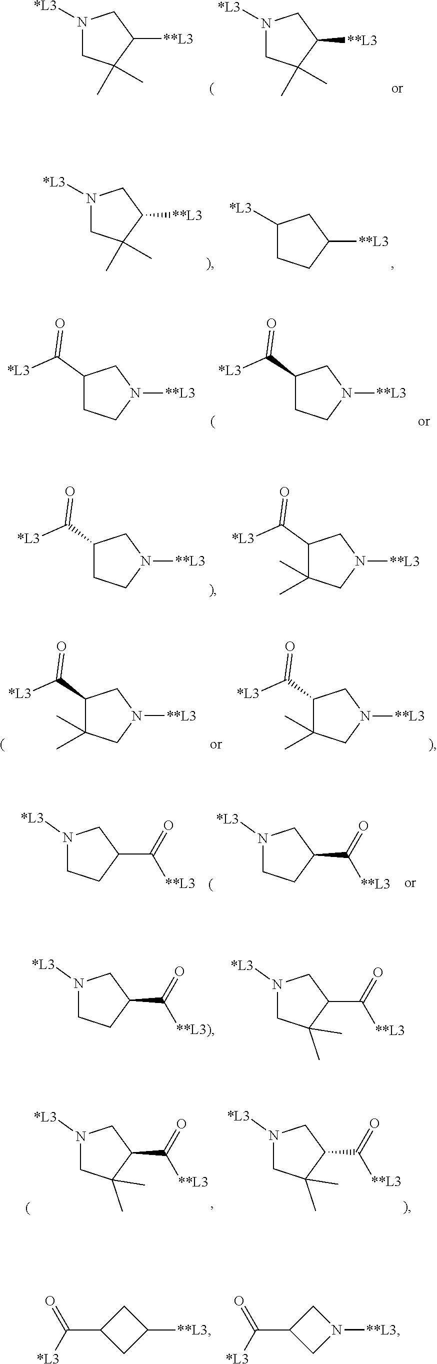

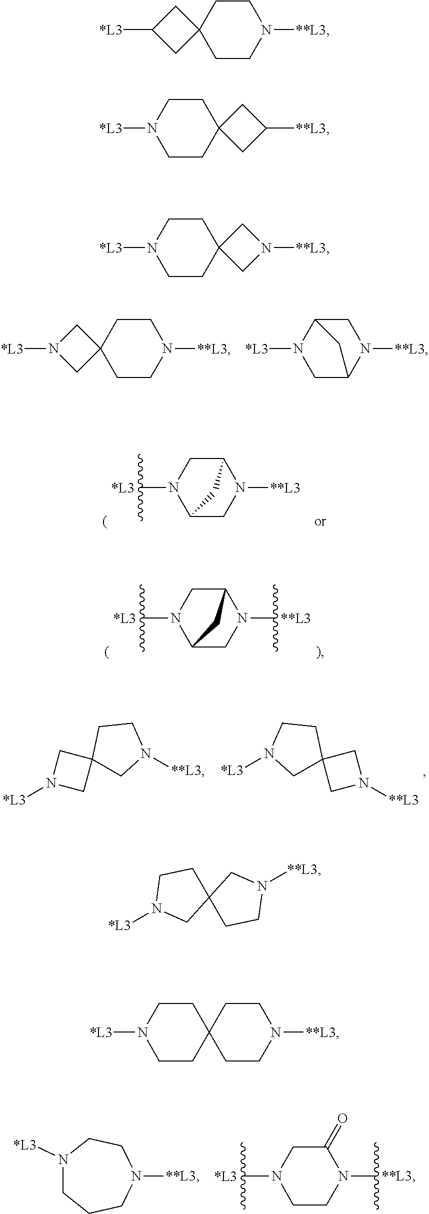

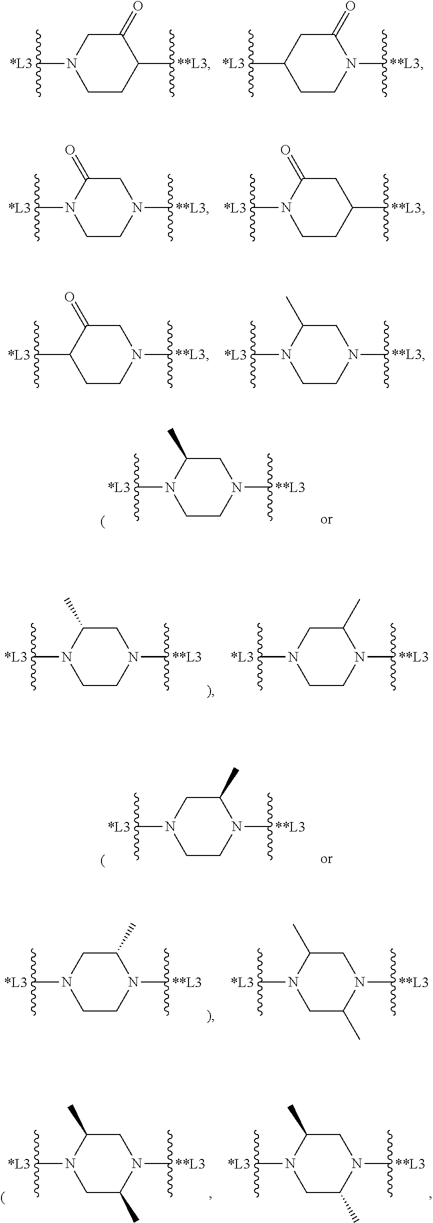

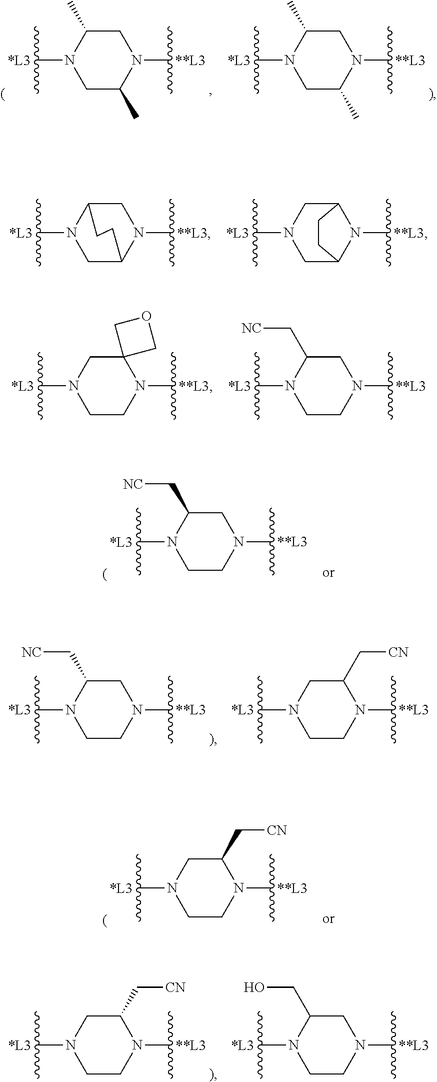

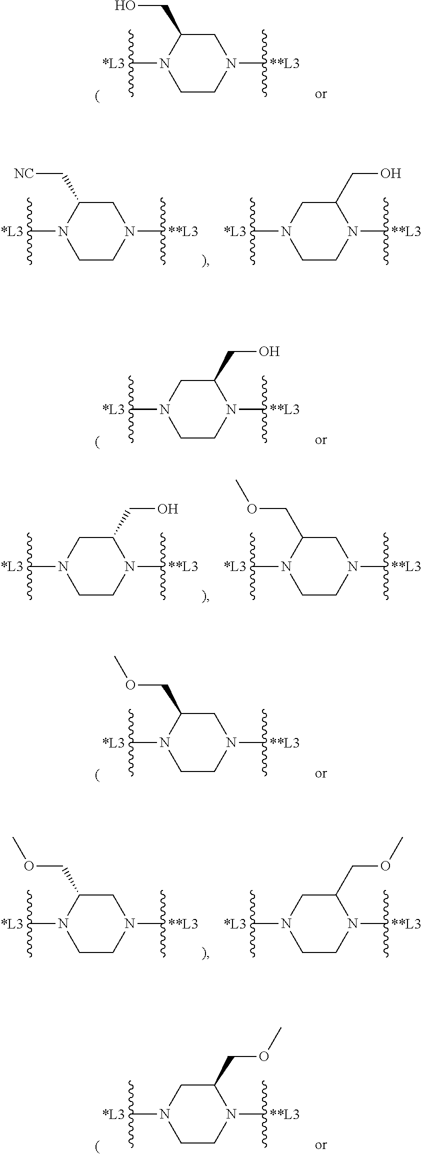

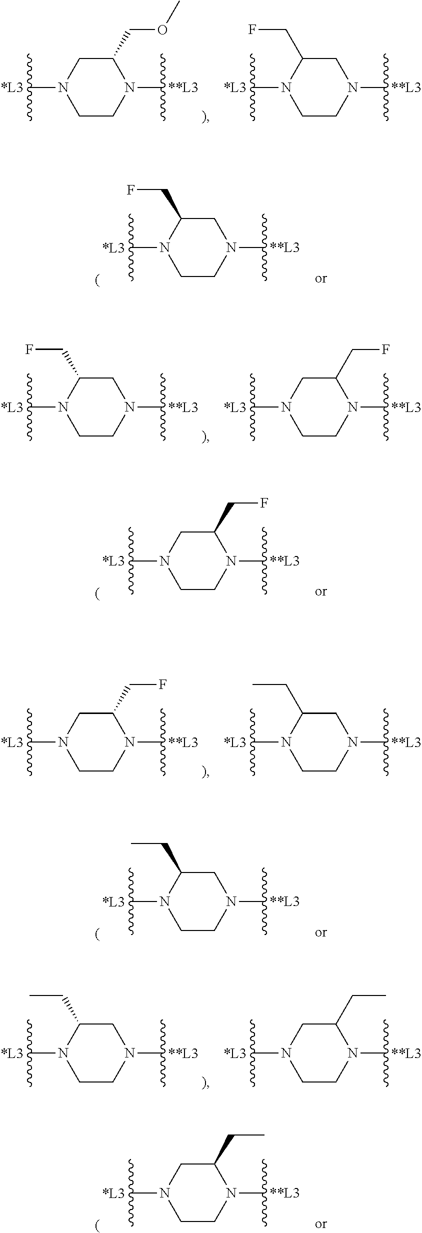

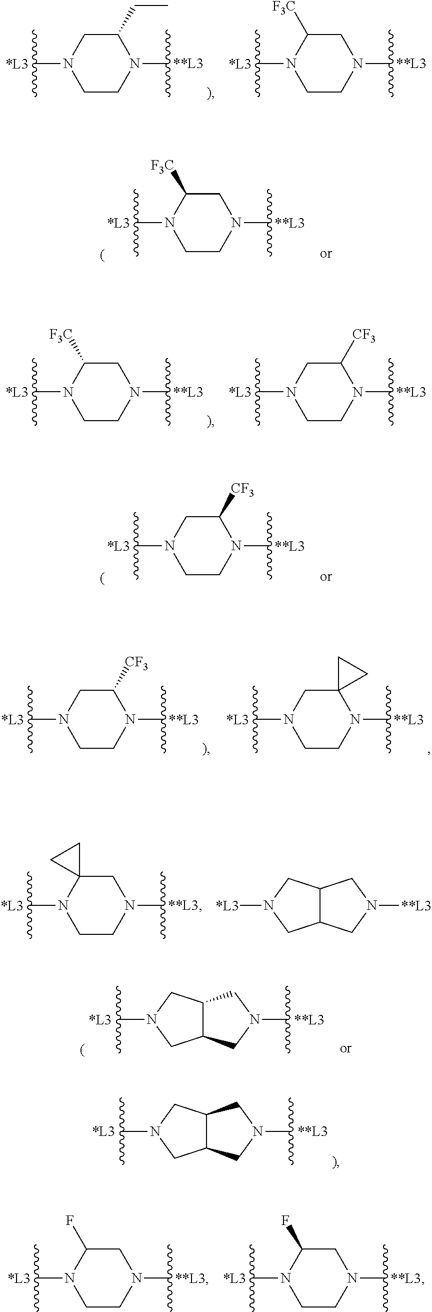

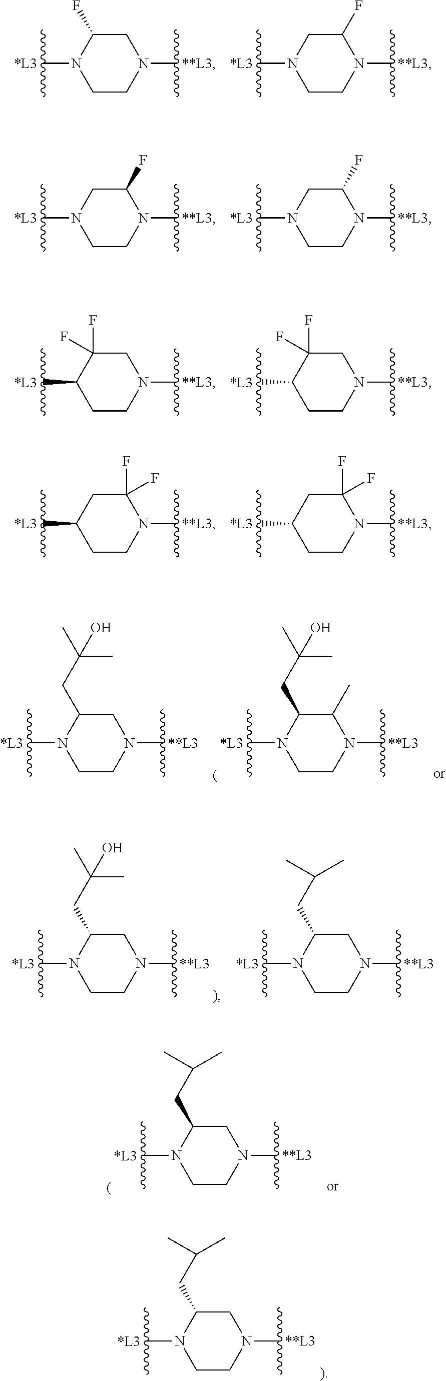

Aspect 26. The compound of any one of the preceding Aspects, wherein

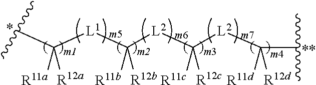

moiety is selected from

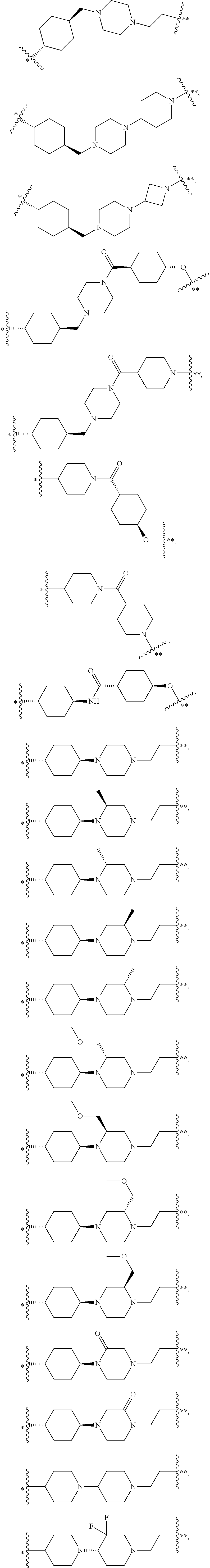

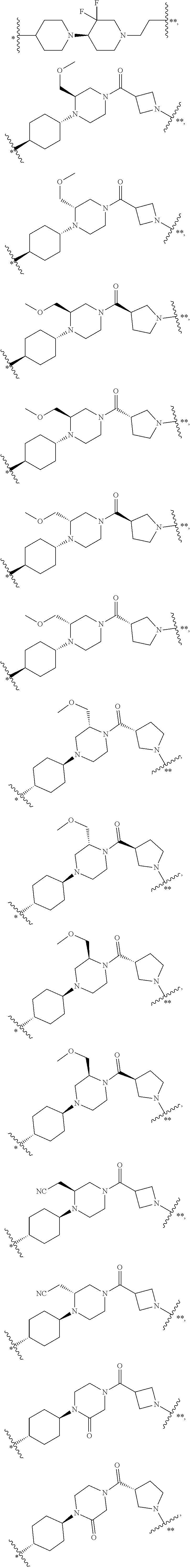

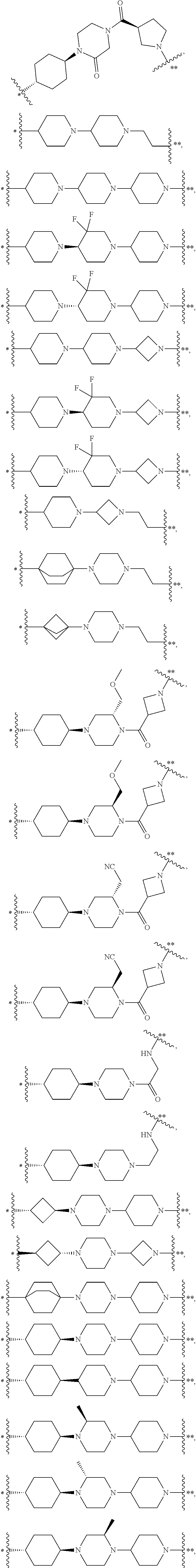

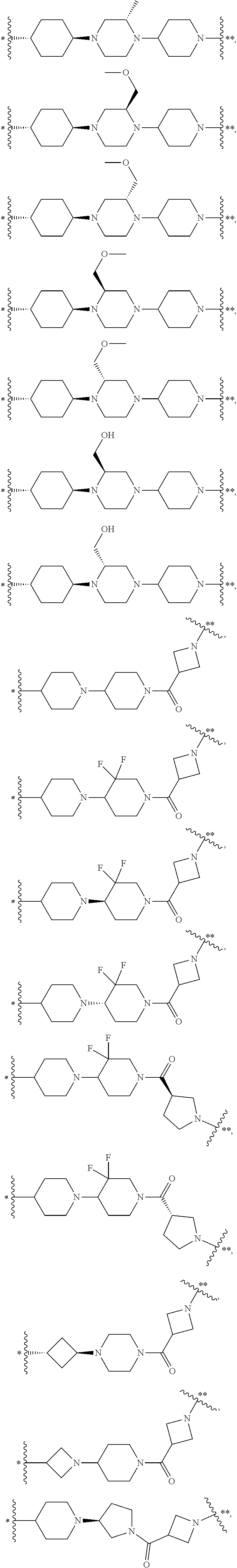

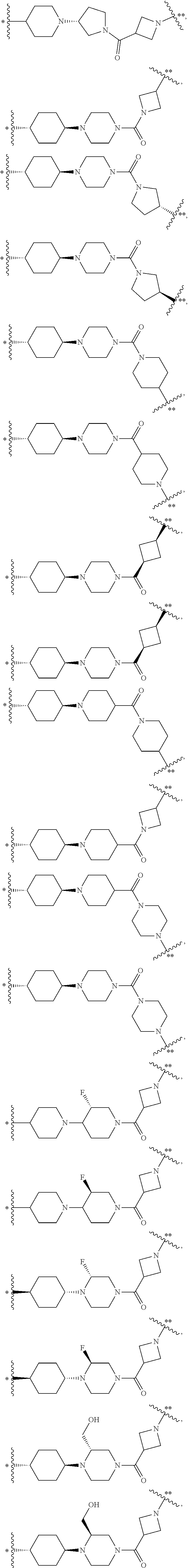

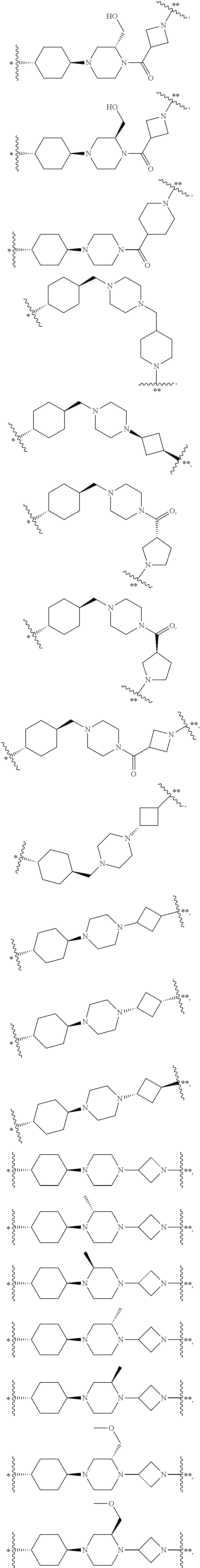

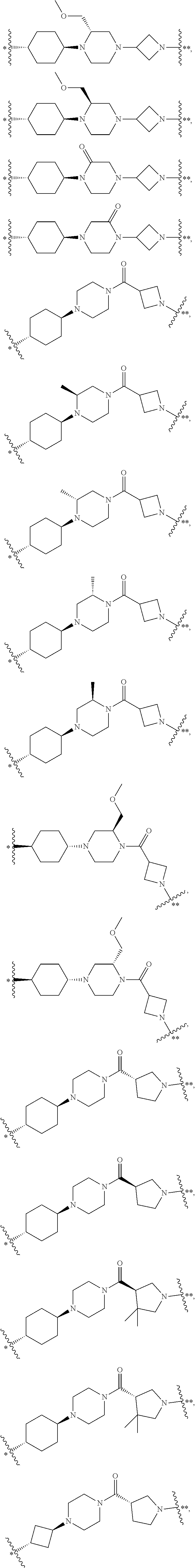

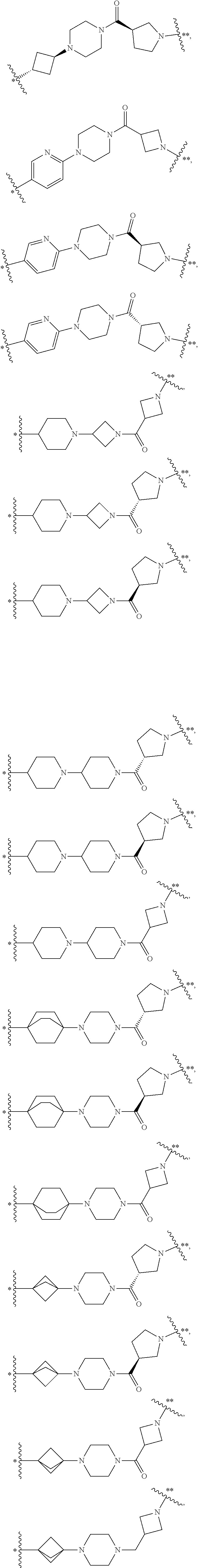

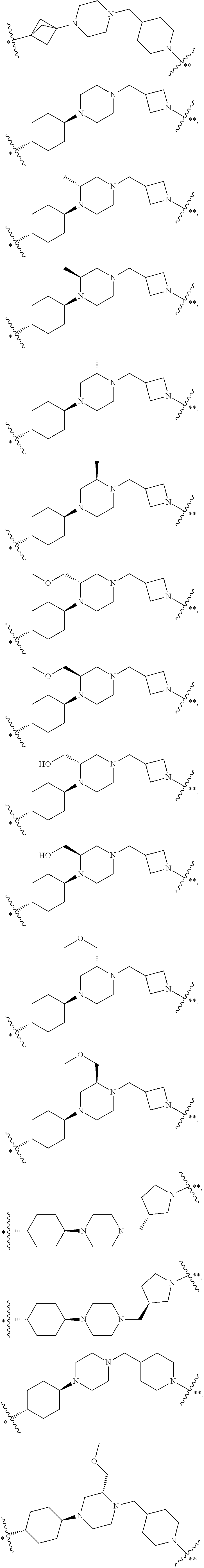

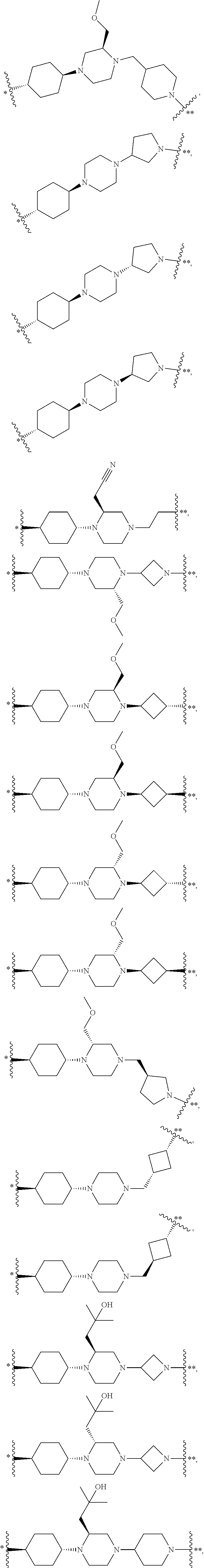

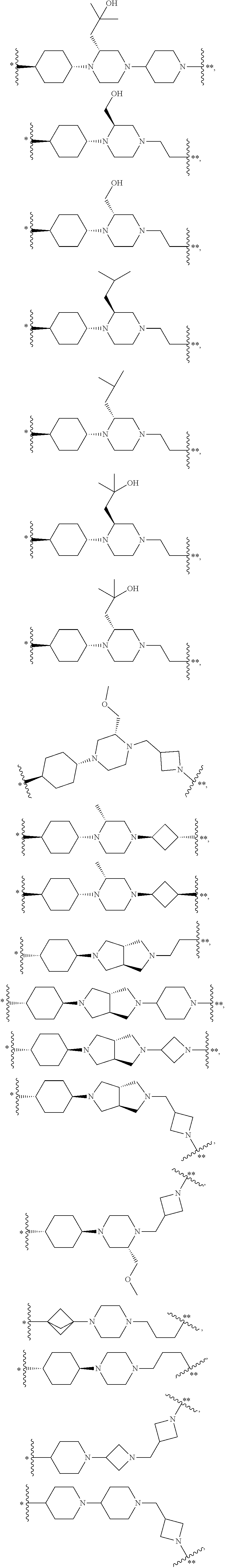

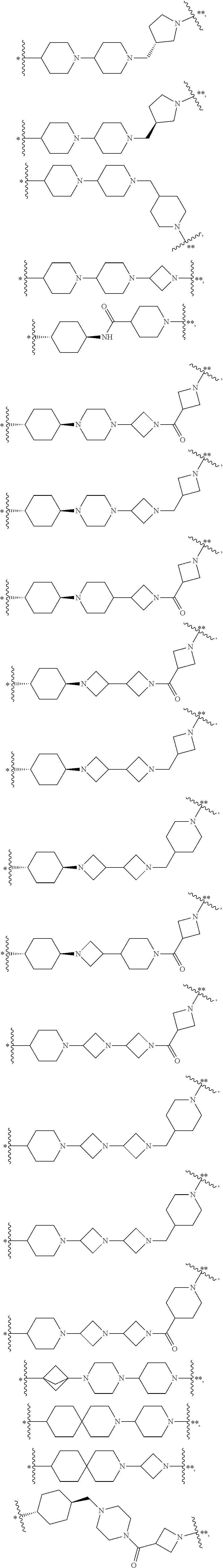

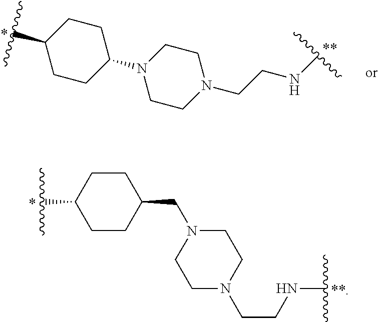 [SMILES: *NCCN1CCN([C@H]2CC[C@H](*)CC2)CC1 ; *NCCN1CCN(C[C@H]2CC[C@H](*)CC2)CC1]

preferably,

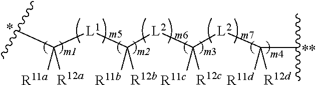

moiety is selected from

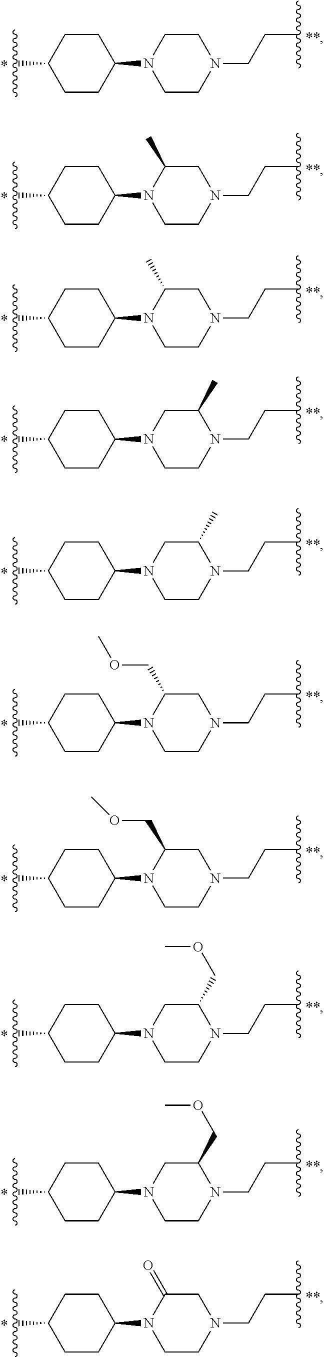

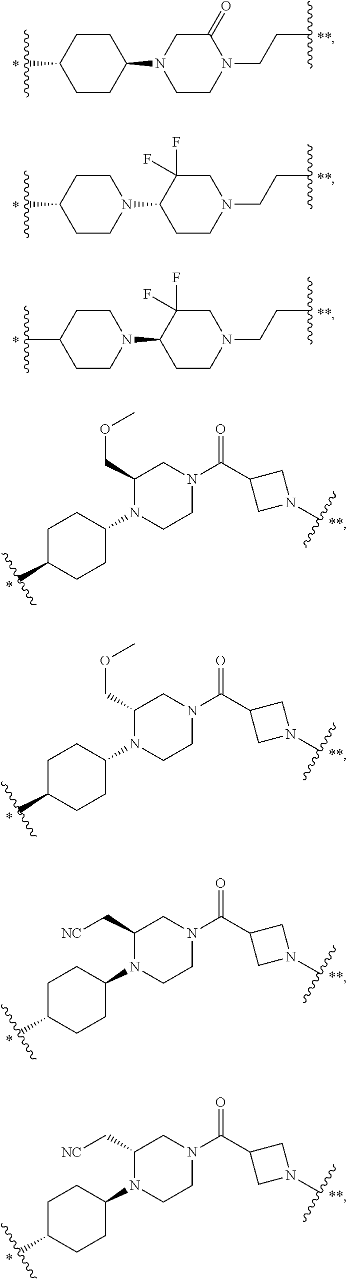

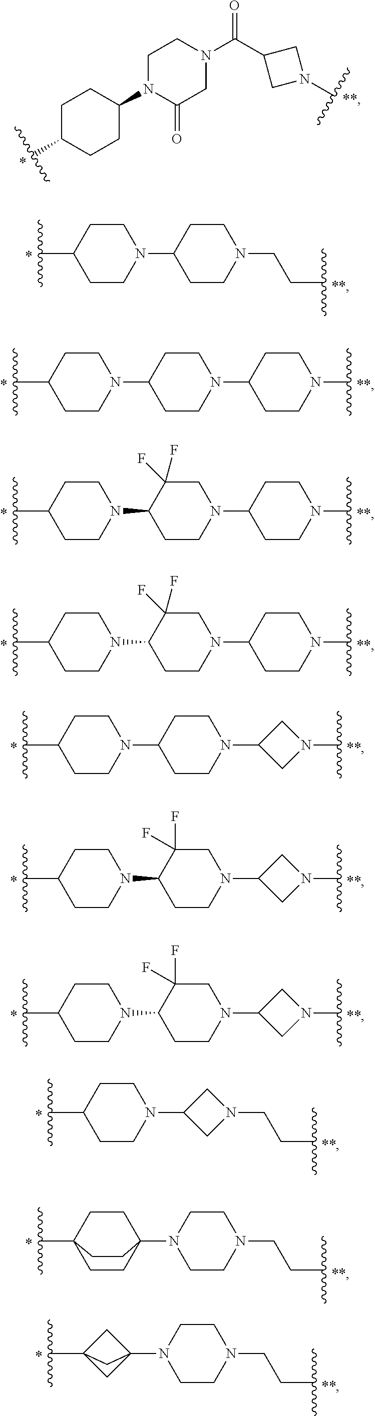

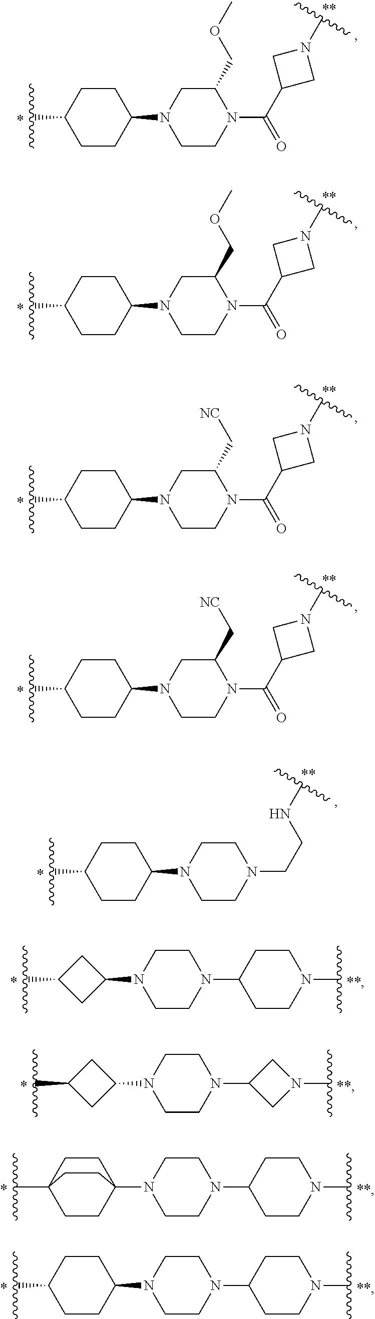

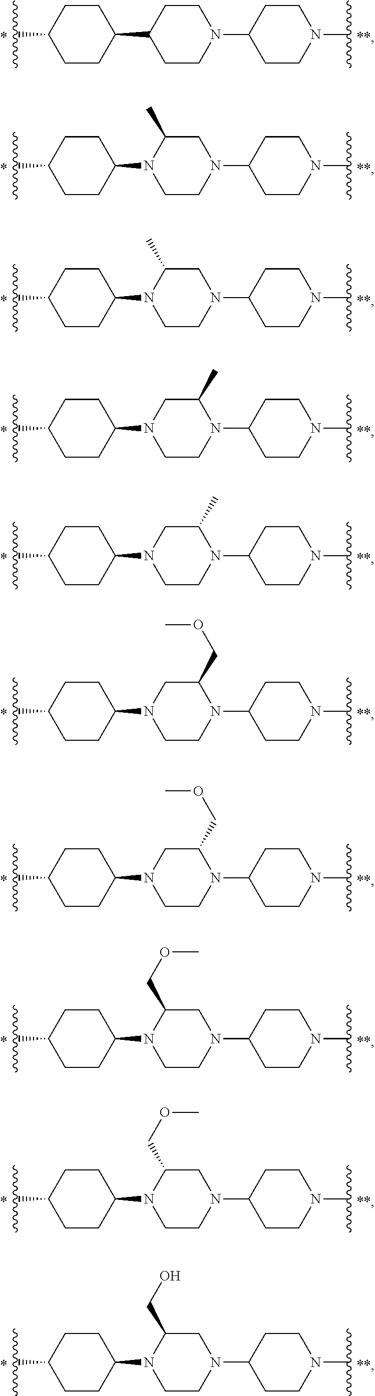

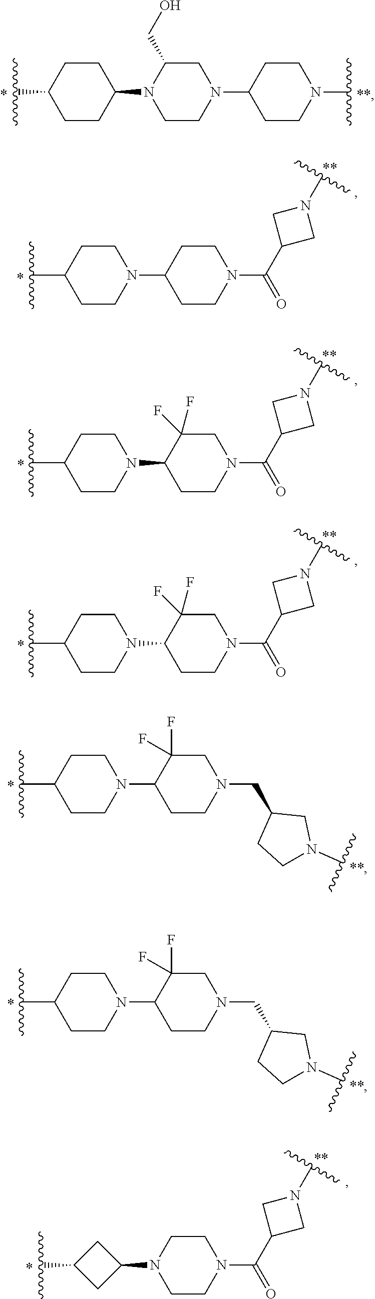

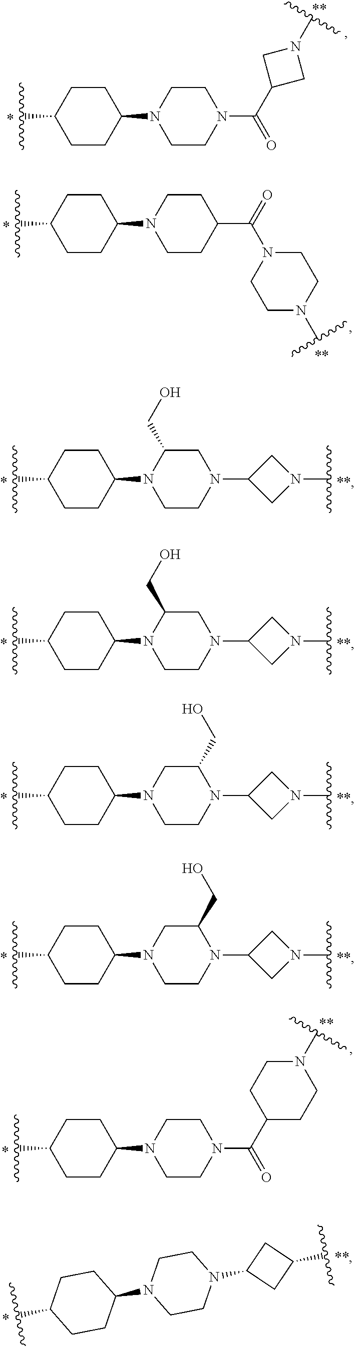

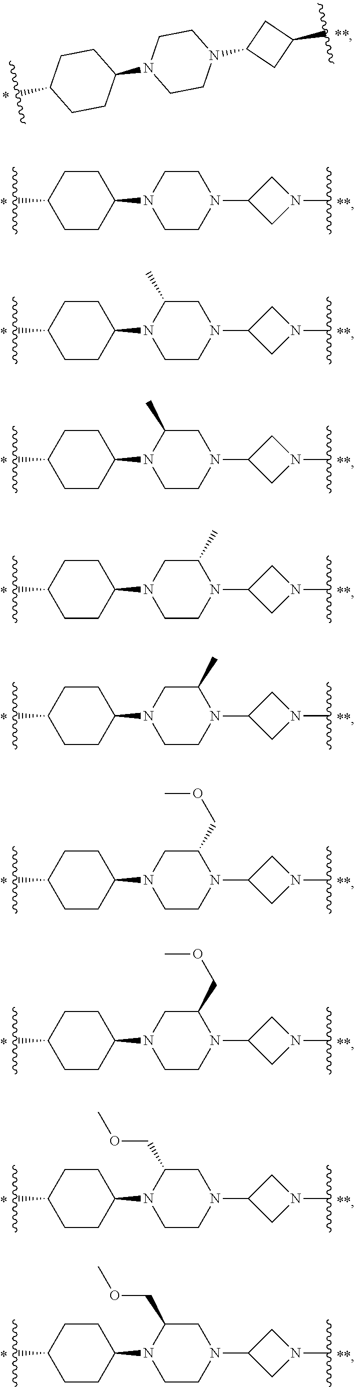

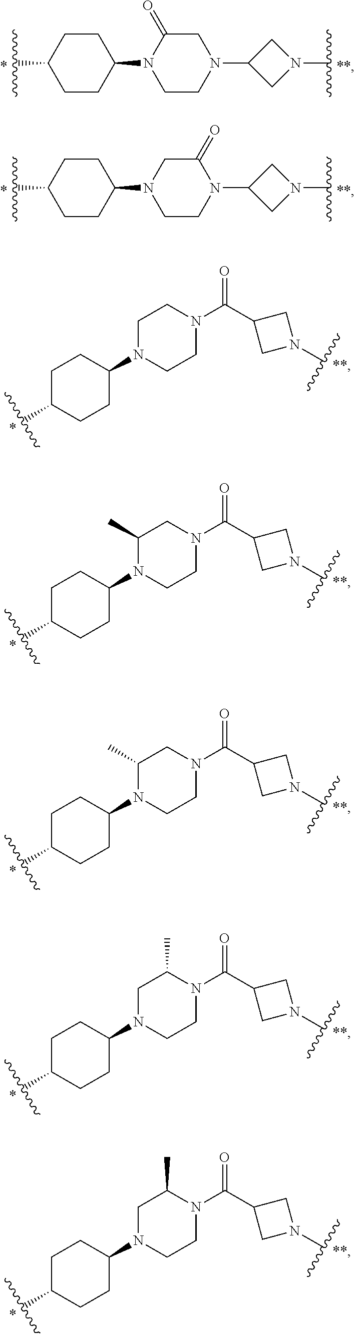

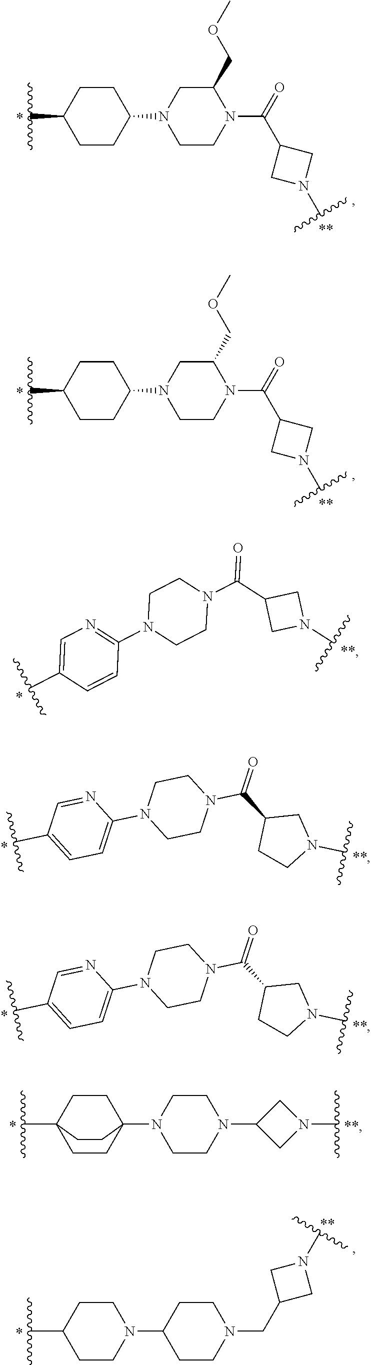

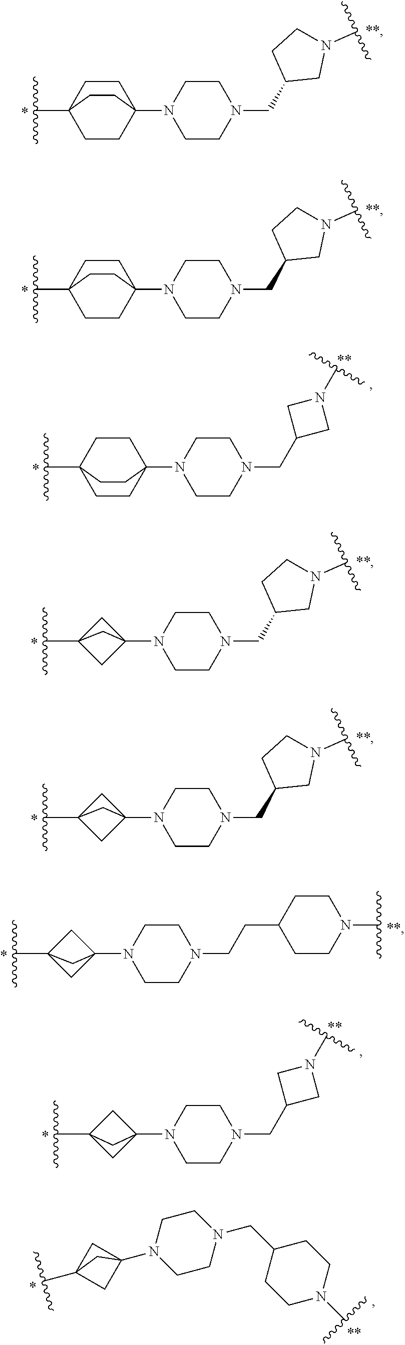

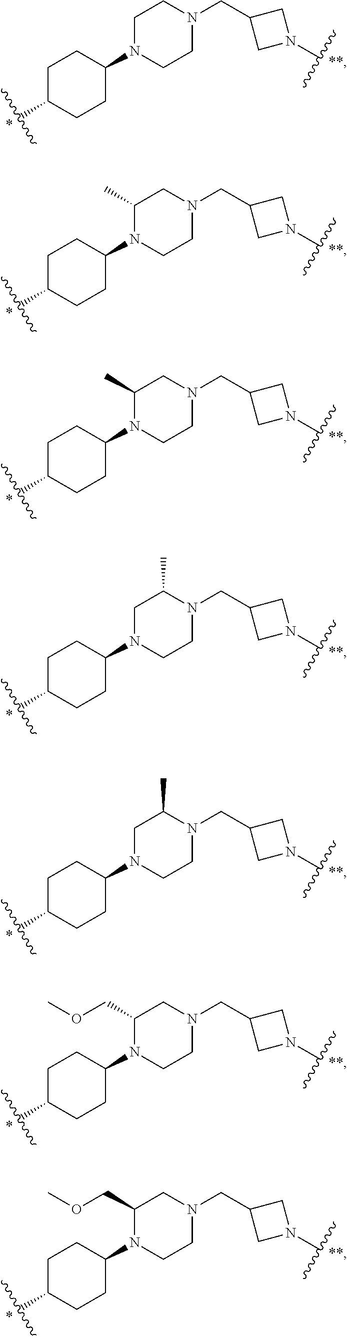

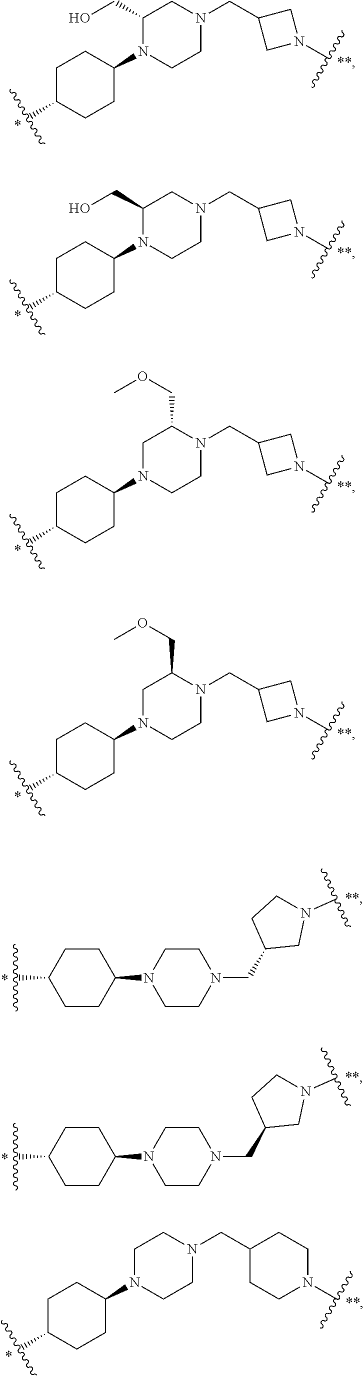

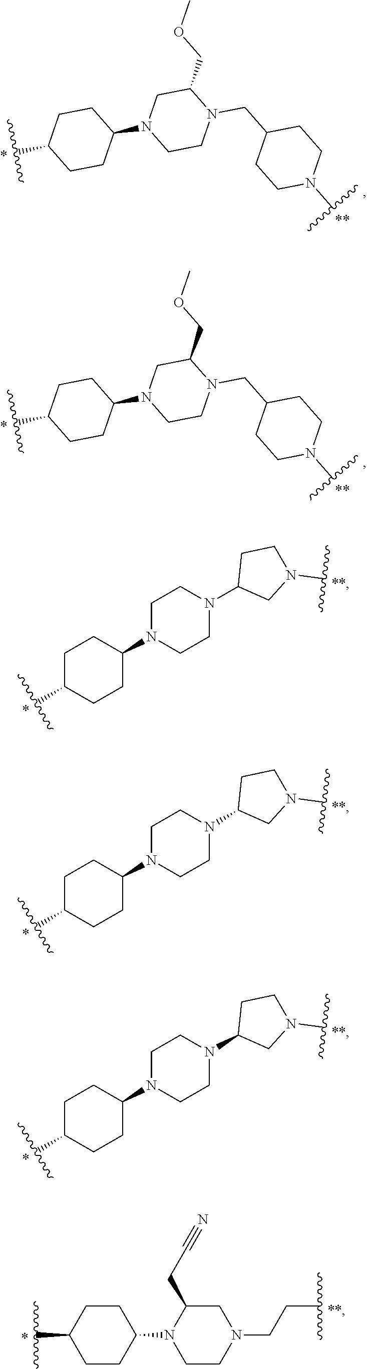 [SMILES: *N1CCC(CN2CCN([C@H]3CC[C@H](*)CC3)C[C@@H]2COC)CC1 ; *N1CCC(CN2CCN([C@H]3CC[C@H](*)CC3)C[C@H]2COC)CC1 ; *N1CCC(N2CCN([C@H]3CC[C@H](*)CC3)CC2)C1 ; *N1CC[C@@H](N2CCN([C@H]3CC[C@H](*)CC3)CC2)C1 ; *N1CC[C@H](N2CCN([C@H]3CC[C@H](*)CC3)CC2)C1 ; *CCN1CCN([C@H]2CC[C@H](*)CC2)[C@@H](CC#N)C1]

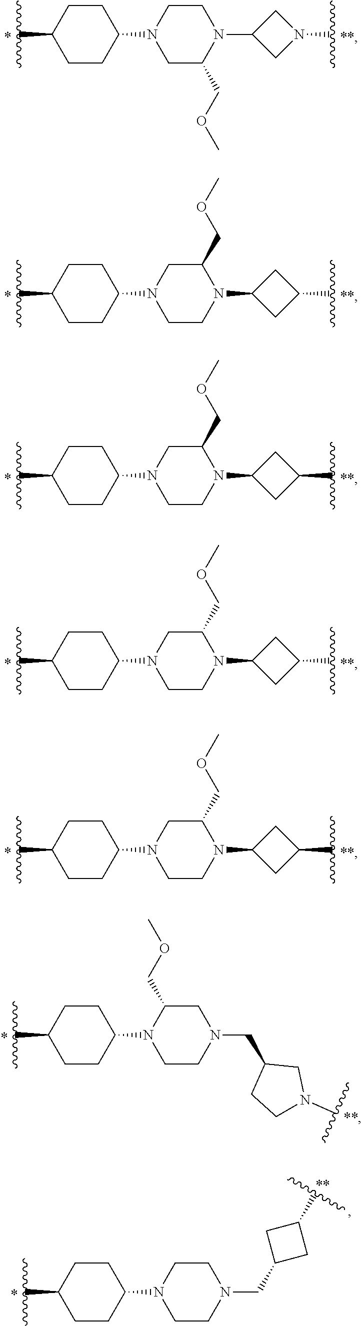

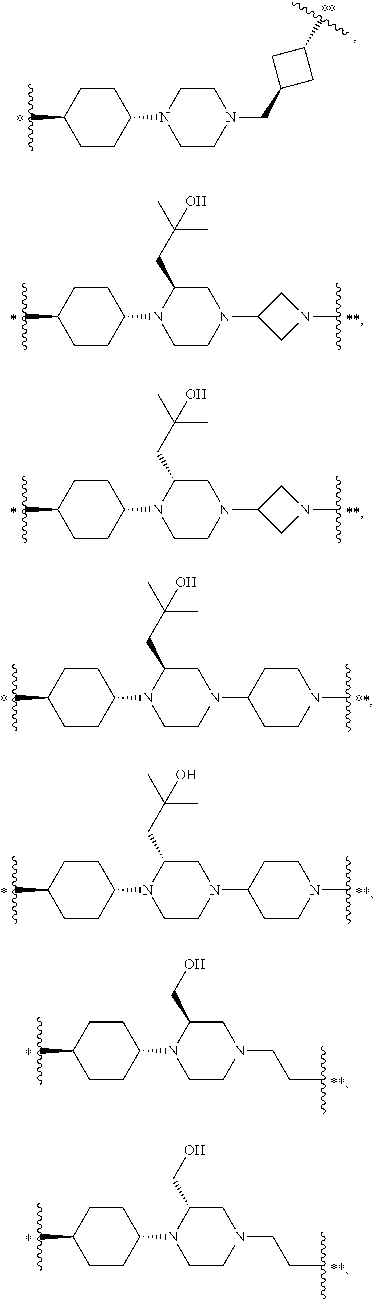

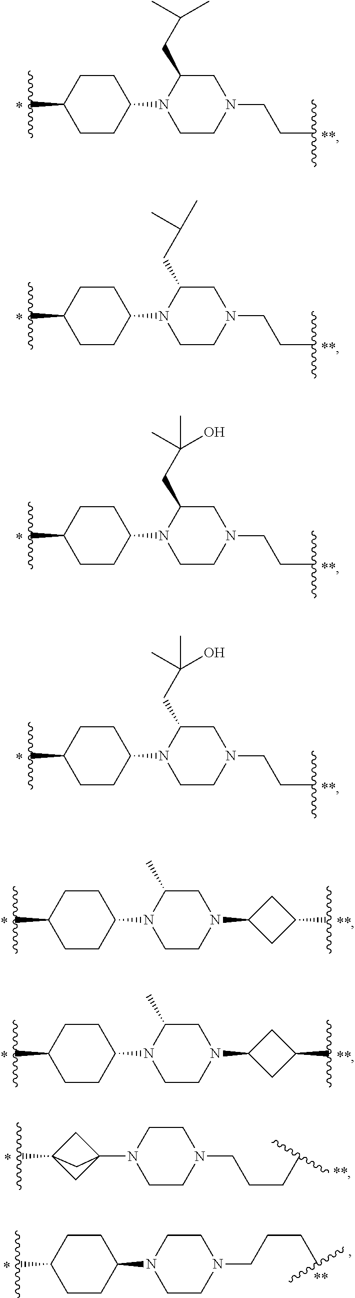

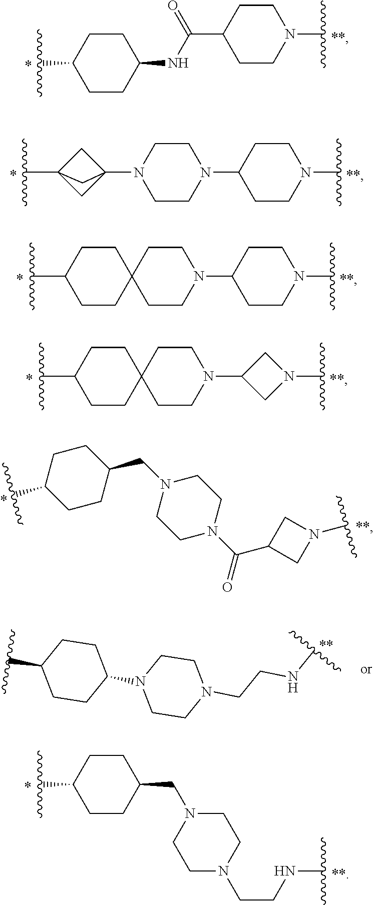

Aspect 27. The compound of any one of the preceding Aspects, wherein L⁴ is independently selected from a single bond, —O—, —NR^{a}—, —(CR^{a}R^{b})ₙ₈—, —O(CR^{a}R^{b})ₙ₈—, —NR^{a}(CR^{a}R^{b})ₙ₈— or —C(O)—;
at each occurrence, R^{a} and R^{b} are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 28. The compound of any one of the preceding Aspects, wherein L⁴ is independently selected from a single bond or —NH—.

Aspect 29. The compound of any one of the preceding Aspects, wherein X⁷ is independently selected from —CR^{a}, or N;
R^{a} is independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 30. The compound of any one of the preceding Aspects, wherein X⁷ is independently selected from —CH, —C(CH₃), or N; preferably X⁷ is independently selected from —CH.

Aspect 31. The compound of any one of the preceding Aspects, wherein X⁸ is independently selected from —NR^{a}—, —O—, —S— and —CR^{a}R^{b}—;
at each occurrence, R^{a} and R are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 32. The compound of any one of the preceding Aspects, wherein X⁸ is independently selected from —NH— and —CH₂—; preferably X⁸ is independently selected from —CH₂—.

Aspect 33. The compound of any one of the preceding Aspects, wherein [symbol] is selected from

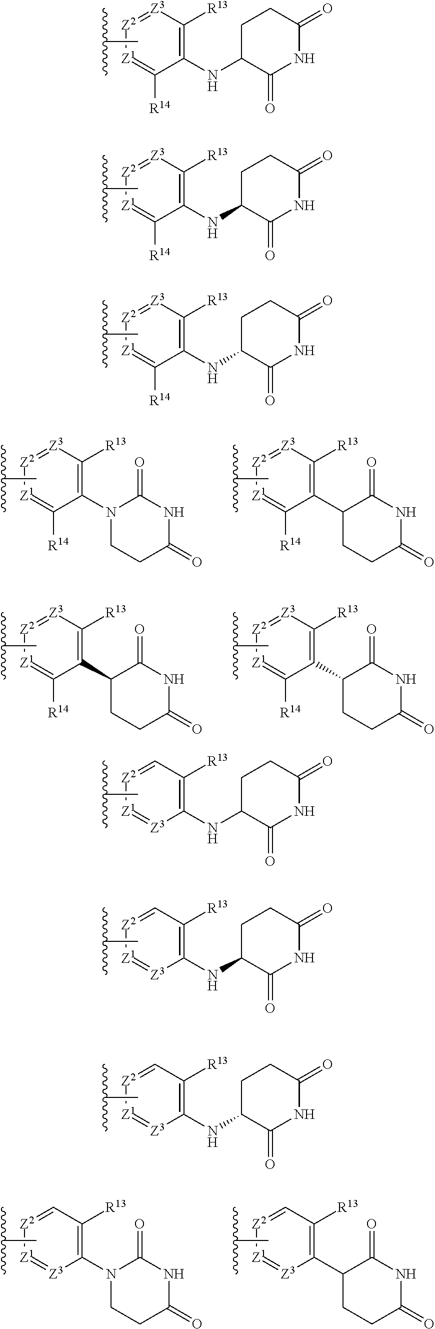

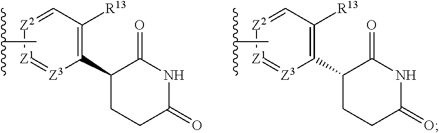

preferably, [symbol] is selected from

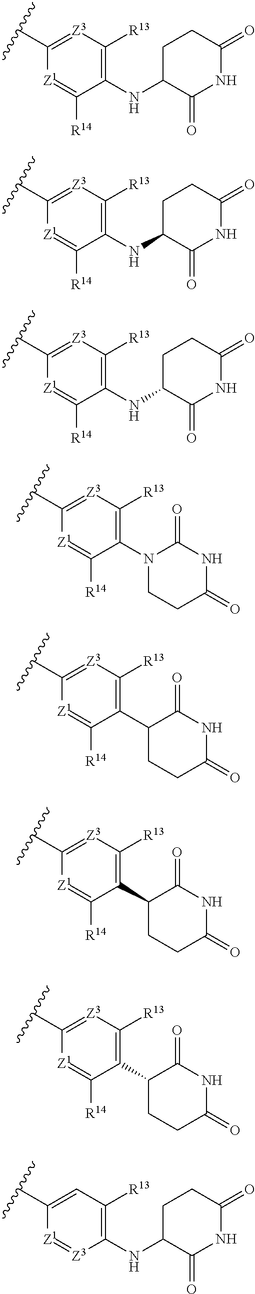

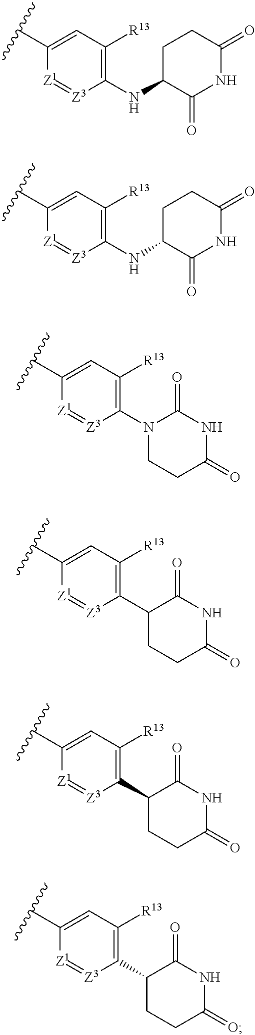

more preferably, [symbol] is selected from

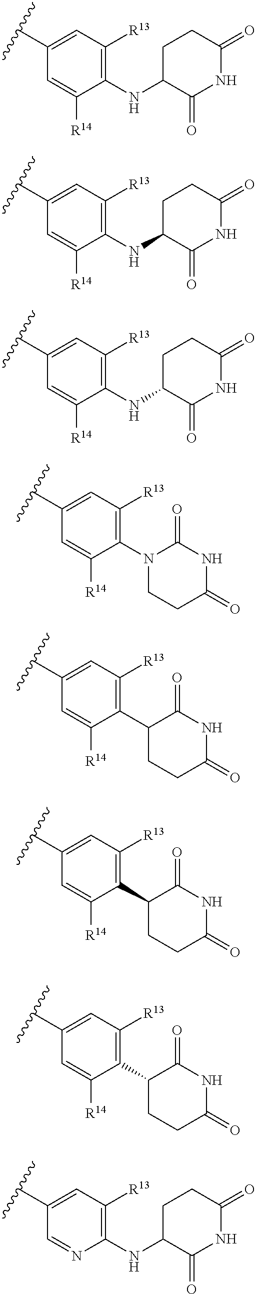

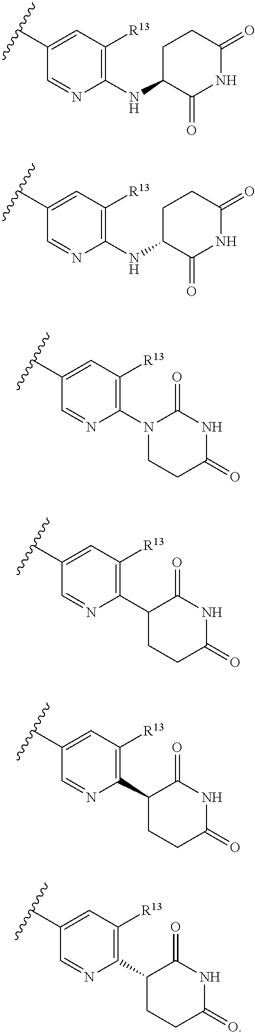

Aspect 34. The compound of any one of the preceding Aspects, wherein at most one of Z¹, Z² and Z³ is N.

Aspect 35. The compound of any one of the preceding Aspects, wherein Z¹, Z² and Z³ are each independently CR^{z}.

Aspect 36. The compound of any one of the preceding Aspects, wherein R^{Z}, at each occurrence, is independently selected from hydrogen, —F, —Cl, —Br, —I, methyl, ethyl, propyl, butyl, pentyl, hexyl, heptyl, octyl, —NR^{Za}R^{Zb}, —OR^{Za}, —SR^{Za}, cyclopropyl, cyclobutyl, cyclopentyl, cyclohexyl, cycloheptyl, cyclooctyl, 3- to 8-membered heterocyclyl or CN; each of methyl, ethyl, propyl, butyl, pentyl, hexyl, heptyl, octyl, cyclopropyl, cyclobutyl, cyclopentyl, cyclohexyl, cycloheptyl, cyclooctyl or 3- to 8-membered heterocyclyl is optionally substituted with at least one R^{Zc};
R^{Za} and R^{Zb} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{Zd};
R^{Zc} and R^{Zd} are each independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptoxy, octyloxy, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 37. The compound of any one of the preceding Aspects, wherein R^{z} is selected from H, —CH₃, —C₂H₅, F, —CH₂F, —CHF₂, —CF₃, —OCH₃, —OC₂H₅, —C₃H₇, —OCH₂F, —OCHF₂, —OCH₂CF₃, —OCF₃, —SCF₃, —CF₃ or —CH(OH)CH₃.

Aspect 38. The compound of any one of the preceding Aspects, wherein R¹³ and R¹⁴ are each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptoxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}, —NR^{13a}CO₂R^{13b}, or —NR^{13a}SO₂R^{13b}; each of methyl, ethyl, propyl, butyl, pentyl, hexyl, heptyl, octyl, methoxy, ethoxy, propoxy, butoxy, pentoxy, hexoxy, heptoxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl is optionally substituted with F, Cl, Br, I, methyl, ethyl, propyl, butyl, pentyl, hexyl, heptyl, octyl, methoxy, ethoxy, propoxy, butoxy, pentoxy, hexoxy, heptoxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, oxo, —CN, —OR^{13c}, —SO₂R^{13c}, —SO₂NR^{13c}R^{13a}, —COR^{13c}, —CO₂R^{13c}, —CONR^{13c}R^{13a}, —NR^{13c}R^{13a}, —NR^{13c}COR^{13a}, —NR^{13c}CO₂R^{13a}, or —NR^{13c}SO₂R^{13d};
R^{13a}, R^{13b}, R^{13c} and R^{13d} are each independently hydrogen, methyl, ethyl, propyl, butyl, pentyl, hexyl, heptyl, octyl, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, or 5- to 12-membered heteroaryl.

Aspect 39. The compound of any one of the preceding Aspects, wherein R¹³ and R¹⁴ are each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptoxy, octyloxy, cyclopropyl, cyclobutyl, cyclopentyl, cyclohexyl, cycloheptyl, cyclooctyl, —CN, —CH₂F, —CHF₂, —CF₃, —OCH₂F, —OCHF₂, —OCH₂CF₃, —OCF₃, —SCF₃, or phenyl.

Aspect 40. The compound of any one of the preceding Aspects wherein [symbol] is

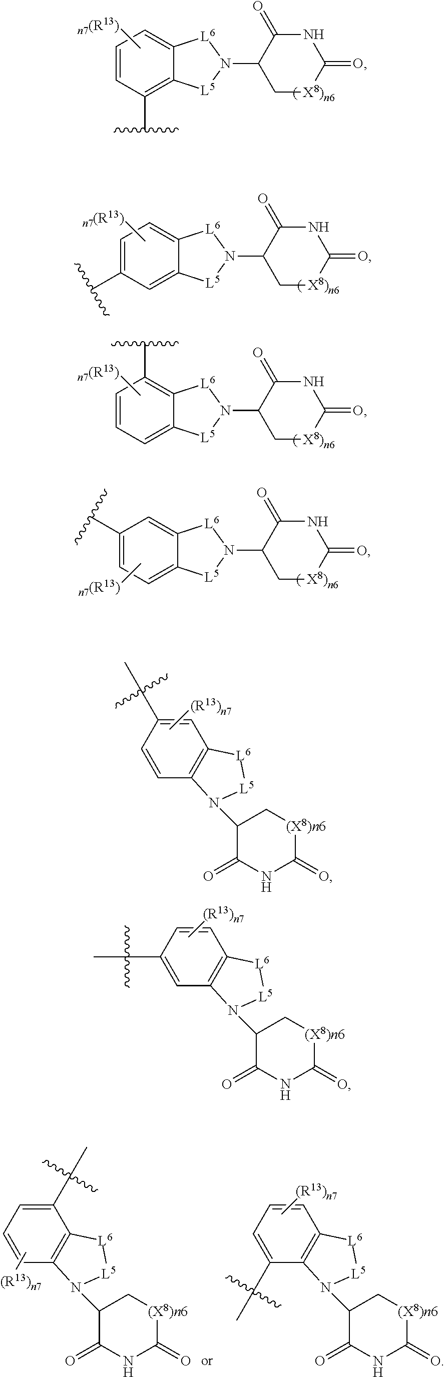

Aspect 41. The compound of any one of the preceding Aspects, wherein L⁵ and L⁶ are each independently selected from a single bond, —O—, —NR^{a}—, —(CR^{a}R^{b})ₙ₈—, —O(CR^{a}R^{b})ₙ₈—, —NR^{a}(CR^{a}R^{b})ₙ₈— or —C(O)—;
X⁸ is —CR^{a}R^{b}—;
at each occurrence, R^{a} and R^{b} are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 42. The compound of any one of the preceding Aspects, wherein L⁵ and L⁶ are each independently a single bond,

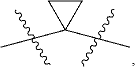 [SMILES: *C1(*)CC1]

—O—, —NH—, —N(CH₂CH₃)—, —CH₂—, —CHF—, —CF₂—, —C(CH₃)₂— or —CO— (preferably L⁵ is —CO— or —CH₂—, and L⁶ is

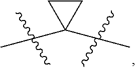 [SMILES: *C1(*)CC1]

—O—, —NH—, —NMe-, —N(CH₂CH₃)—, —CH₂—, —CHF—, —CF₂—, —C(CH₃)₂— or —CO—);
X⁸ is CH₂; and
n6 is 0 or 1.

Aspect 43. The compound of any one of the preceding Aspects, wherein R¹³ is independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptoxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}, —NR^{13a}CO₂R^{13b}, or —NR^{13a}SO₂R^{13b}; each of methyl, ethyl, propyl, butyl, pentyl, hexyl, heptyl, octyl, methoxy, ethoxy, propoxy, butoxy, pentoxy, hexoxy, heptoxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl is optionally substituted with F, Cl, Br, I, methyl, ethyl, propyl, butyl, pentyl, hexyl, heptyl, octyl, methoxy, ethoxy, propoxy, butoxy, pentoxy, hexoxy, heptoxy, octyloxy, cyclopropyl, cyclobutyl, cyclopentyl, cyclohexyl, cycloheptyl, cyclooctyl, —C₂₋₈ alkenyl, —C₂₋₈alkynyl, 3- to 8-membered heterocyclyl, —C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{13c}, —CONR^{13c}R^{13d}, —NR^{13c}R^{13d}, —NR^{13c}COR^{13d}, —NR^{13c}CO₂R^{13d}, or —NR^{13c}SO₂R^{13d};
at each occurrence, R^{13a}, R^{13b}, R^{13c} and R^{13d} are each independently hydrogen, methyl, ethyl, propyl, butyl, pentyl, hexyl, heptyl, octyl, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, or 5- to 12-membered heteroaryl.

Aspect 44. The compound of any one of the preceding Aspects, wherein R¹³ is independently selected from hydrogen, F, Cl, Br, I, CN, —C₁-C₈alkyl, or —C₁-C₈alkoxy; preferably R¹³ is independently selected from hydrogen, F, Cl, Br, I, CN, -Me, -Et, —C₃H₇, —C₄H₉, —OMe, —OEt, —OC₃H₇ or —OC₄H₉;
n₇ is 0, 1 or 2.

Aspect 45. The compound of any one of the preceding Aspects, wherein [symbol] is

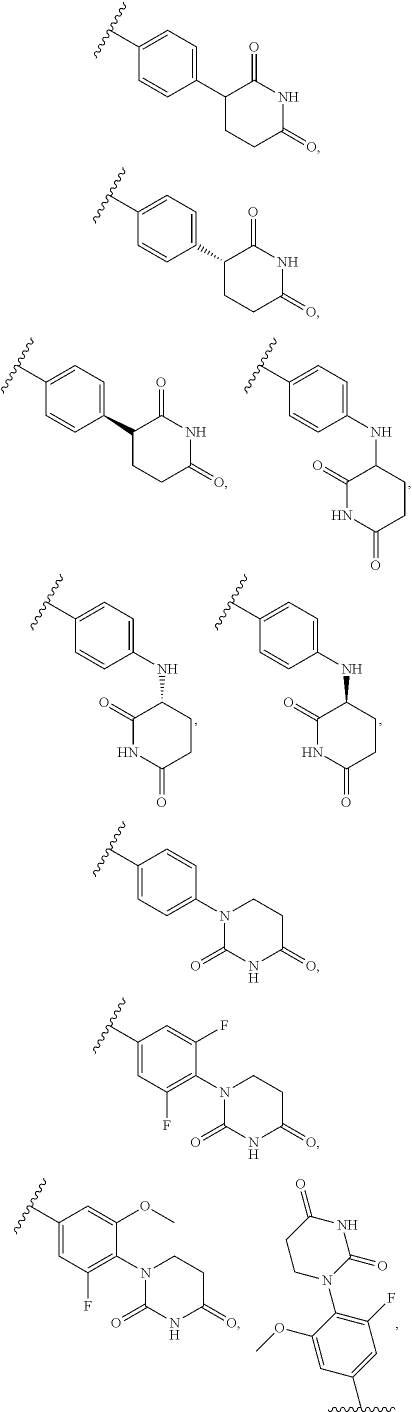

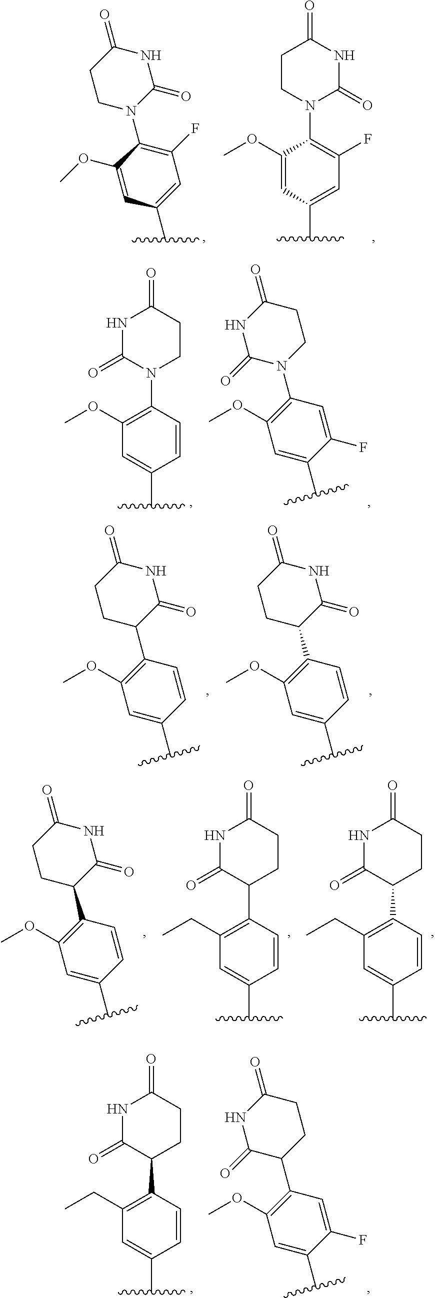

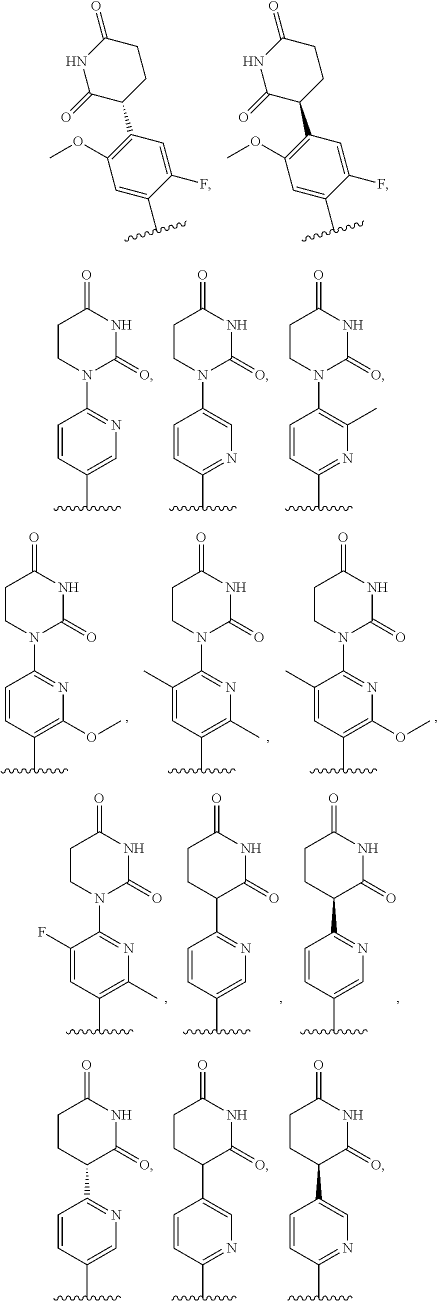

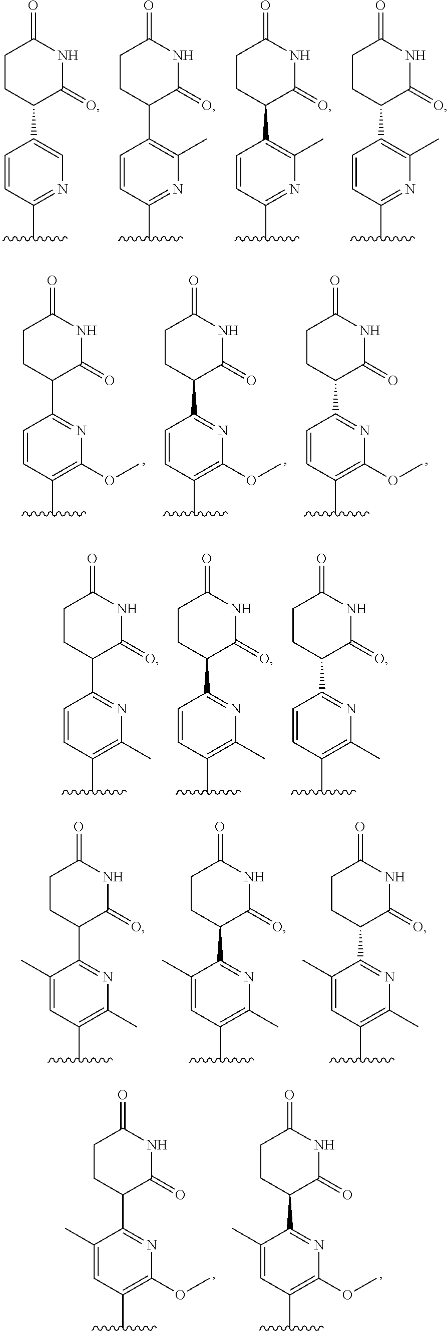

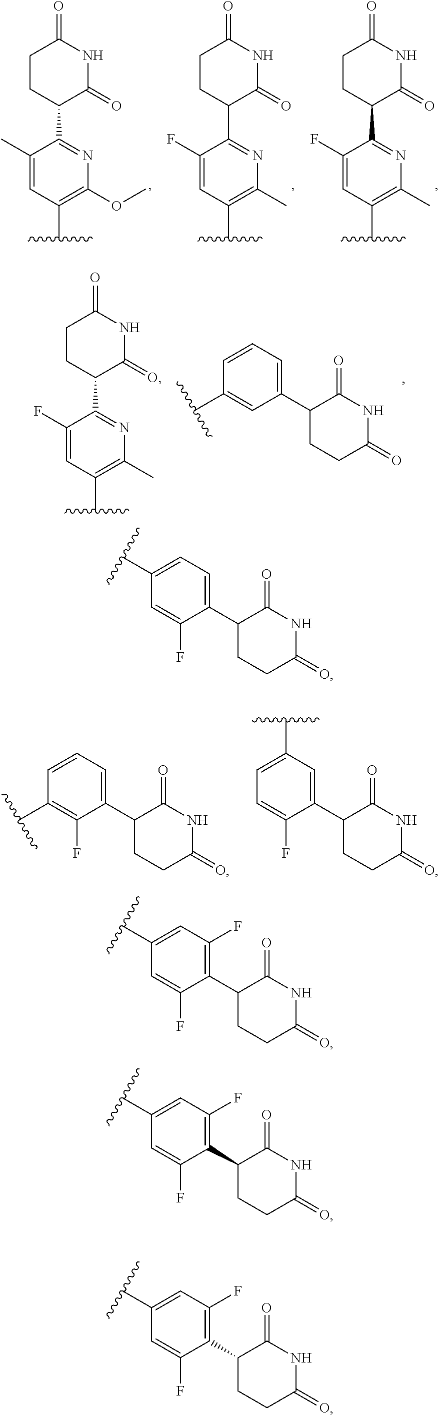

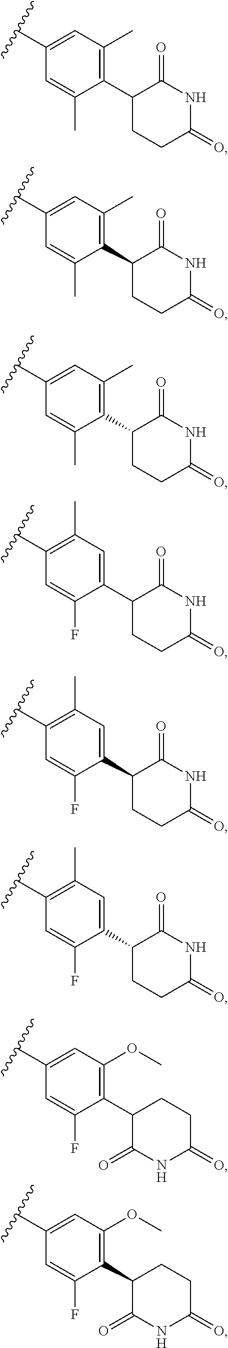

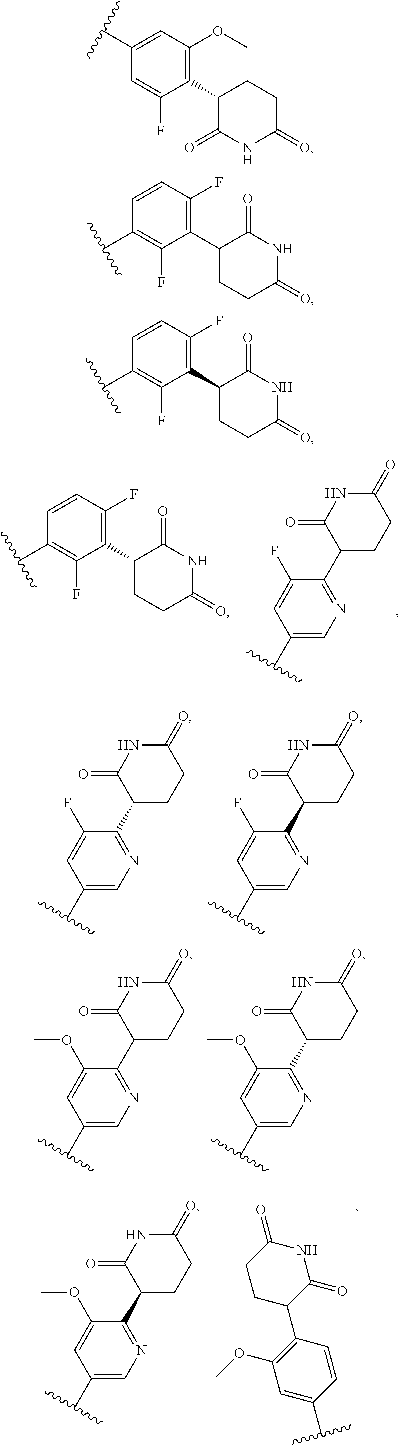

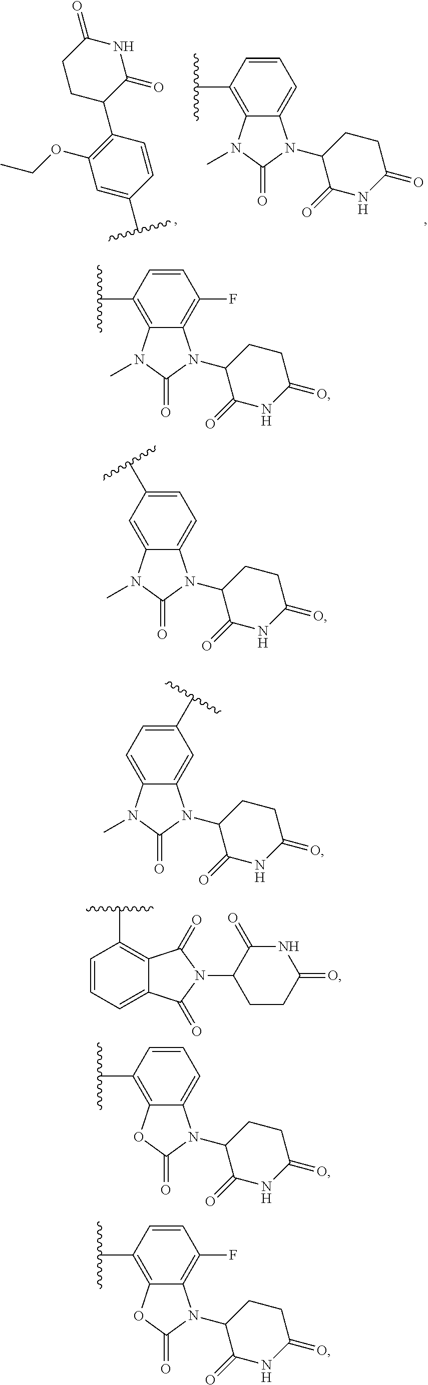

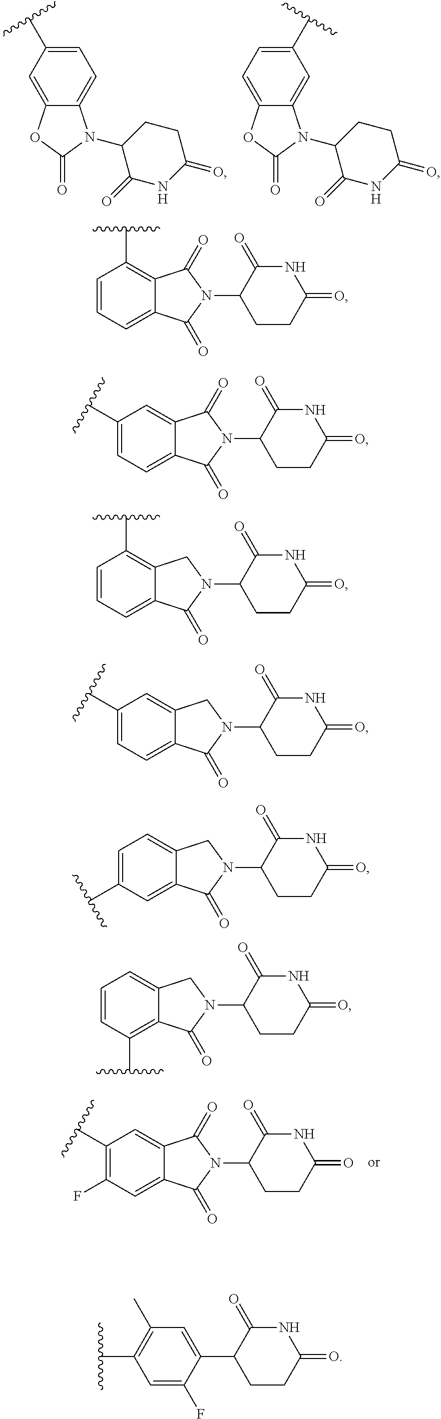

Aspect 46. The compound of any one of the preceding Aspects, wherein the compound is selected from

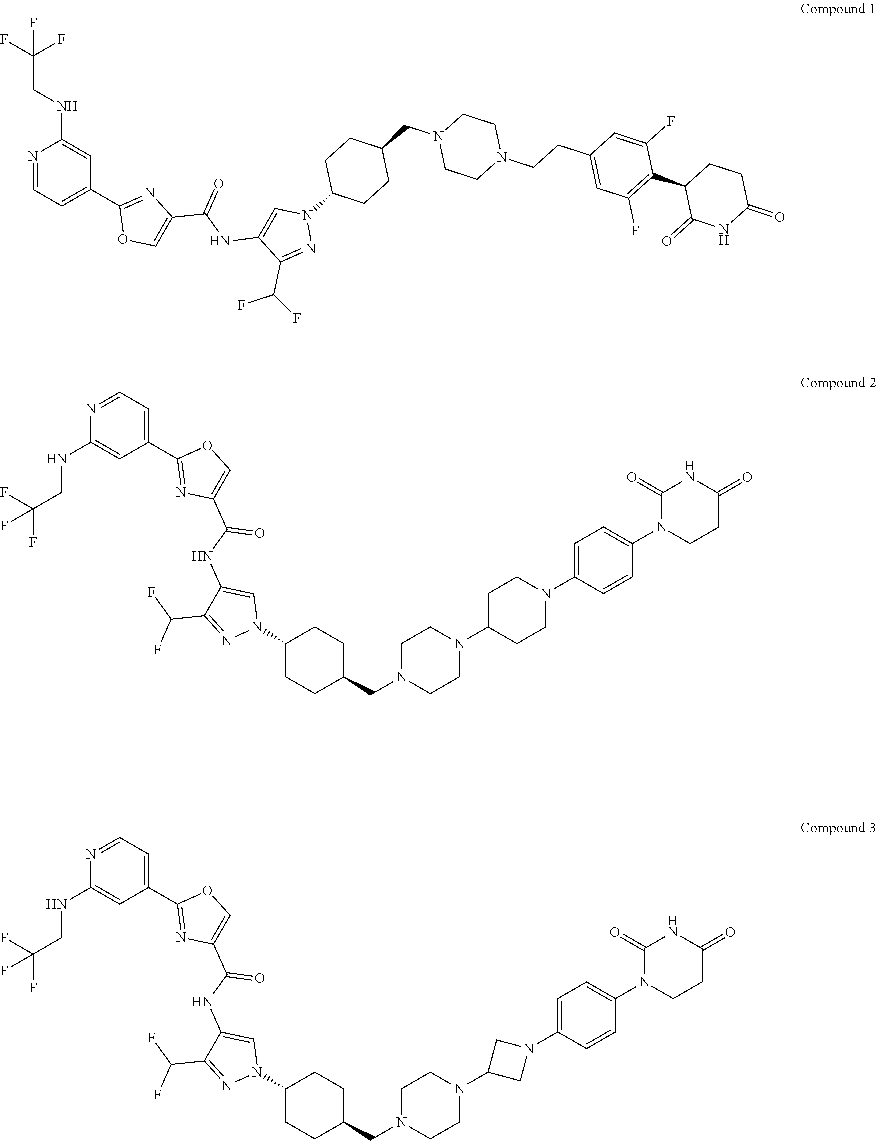 [SMILES: O=C1CC[C@H](c2c(F)cc(CCN3CCN(C[C@H]4CC[C@H](n5cc(NC(=O)c6coc(-c7ccnc(NCC(F)(F)F)c7)n6)c(C(F)F)n5)CC4)CC3)cc2F)C(=O)N1 ; O=C1CCN(c2ccc(N3CCC(N4CCN(C[C@H]5CC[C@H](n6cc(NC(=O)c7coc(-c8ccnc(NCC(F)(F)F)c8)n7)c(C(F)F)n6)CC5)CC4)CC3)cc2)C(=O)N1 ; O=C1CCN(c2ccc(N3CC(N4CCN(C[C@H]5CC[C@H](n6cc(NC(=O)c7coc(-c8ccnc(NCC(F)(F)F)c8)n7)c(C(F)F)n6)CC5)CC4)C3)cc2)C(=O)N1]

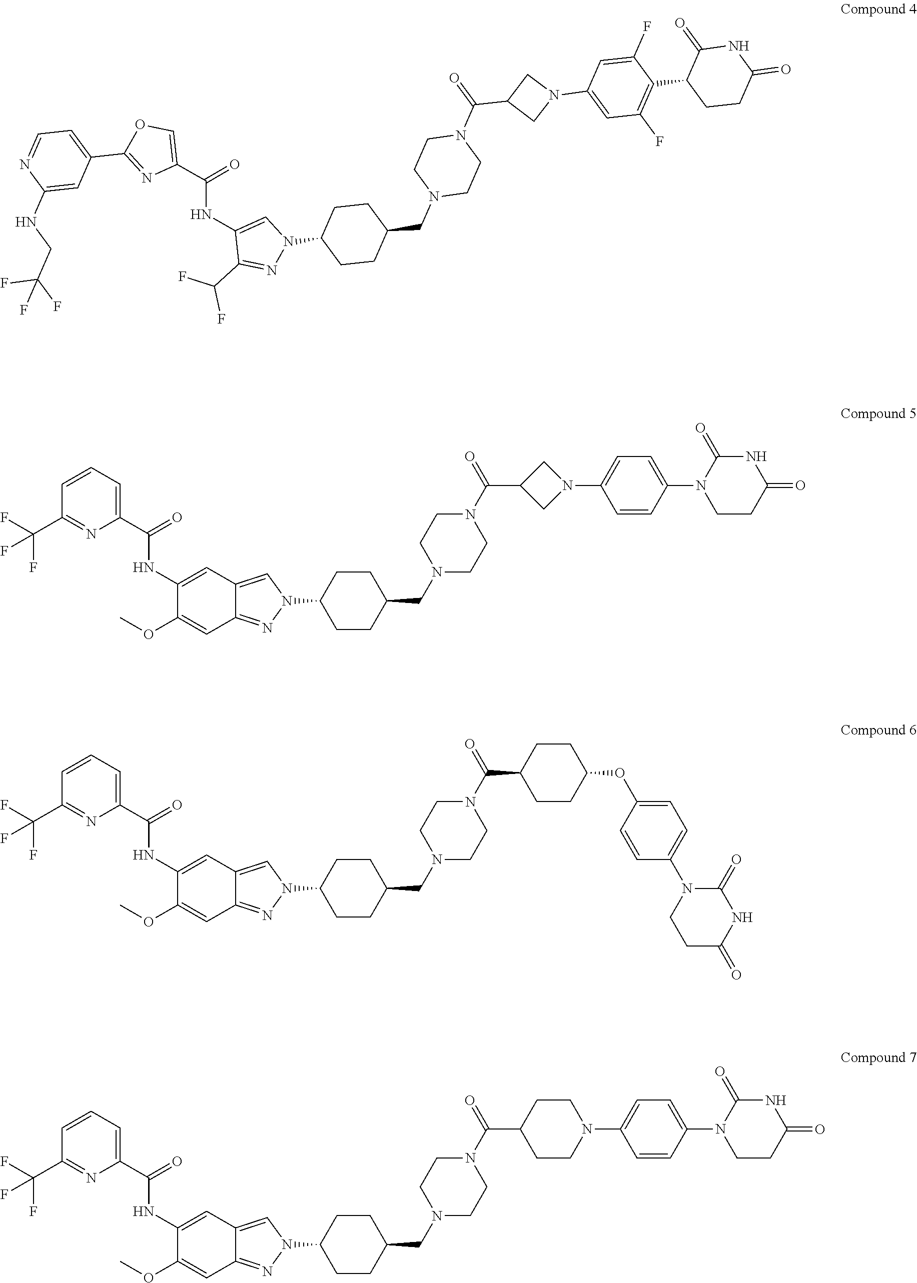 [SMILES: O=C1CC[C@H](c2c(F)cc(N3CC(C(=O)N4CCN(C[C@H]5CC[C@H](n6cc(NC(=O)c7coc(-c8ccnc(NCC(F)(F)F)c8)n7)c(C(F)F)n6)CC5)CC4)C3)cc2F)C(=O)N1 ; COc1cc2nn([C@H]3CC[C@H](CN4CCN(C(=O)C5CN(c6ccc(N7CCC(=O)NC7=O)cc6)C5)CC4)CC3)cc2cc1NC(=O)c1cccc(C(F)(F)F)n1 ; COc1cc2nn([C@H]3CC[C@H](CN4CCN(C(=O)[C@H]5CC[C@H](Oc6ccc(N7CCC(=O)NC7=O)cc6)CC5)CC4)CC3)cc2cc1NC(=O)c1cccc(C(F)(F)F)n1 ; COc1cc2nn([C@H]3CC[C@H](CN4CCN(C(=O)C5CCN(c6ccc(N7CCC(=O)NC7=O)cc6)CC5)CC4)CC3)cc2cc1NC(=O)c1cccc(C(F)(F)F)n1]

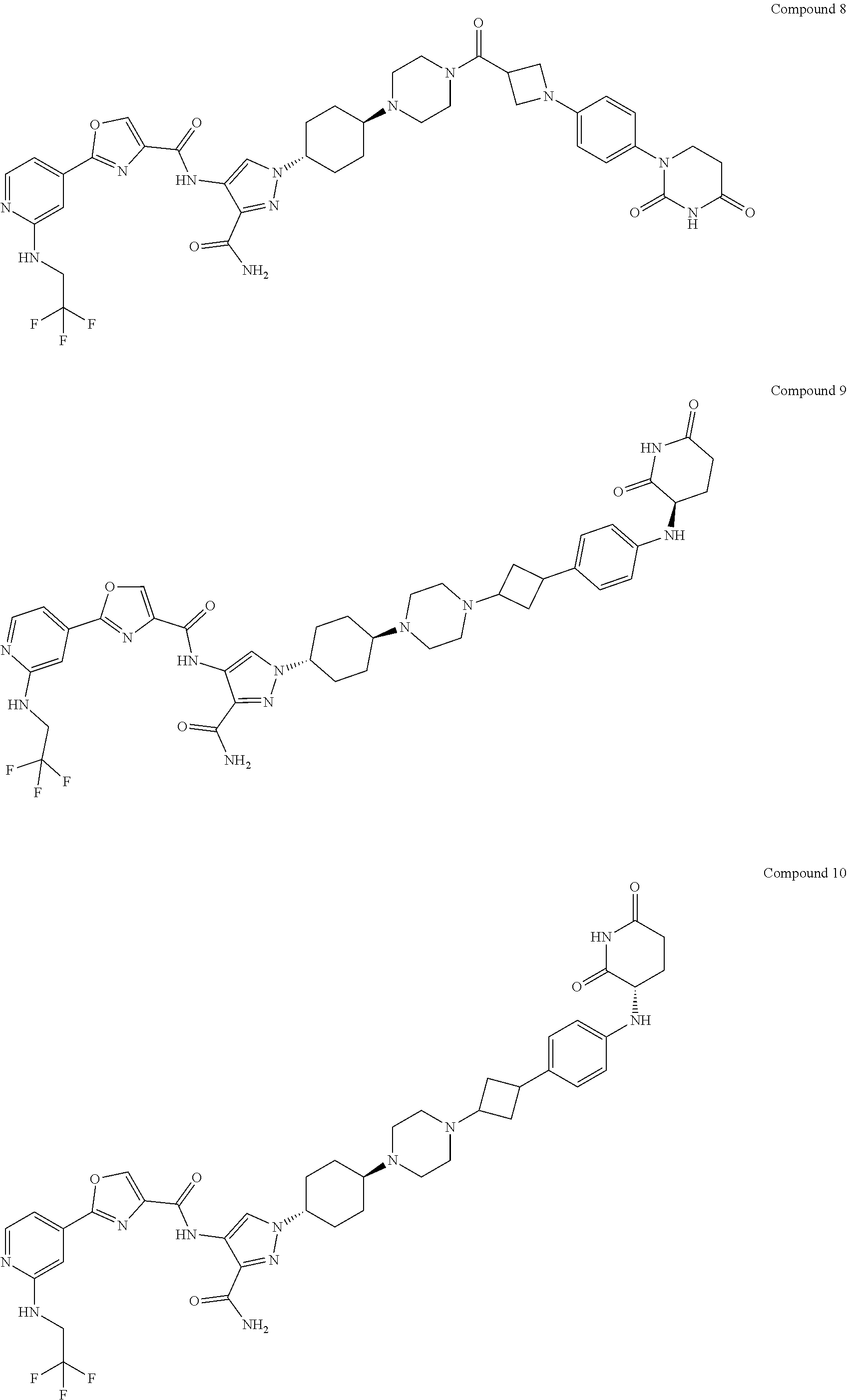 [SMILES: NC(=O)c1nn([C@H]2CC[C@H](N3CCN(C(=O)C4CN(c5ccc(N6CCC(=O)NC6=O)cc5)C4)CC3)CC2)cc1NC(=O)c1coc(-c2ccnc(NCC(F)(F)F)c2)n1 ; NC(=O)c1nn([C@H]2CC[C@H](N3CCN(C4CC(c5ccc(N[C@@H]6CCC(=O)NC6=O)cc5)C4)CC3)CC2)cc1NC(=O)c1coc(-c2ccnc(NCC(F)(F)F)c2)n1 ; NC(=O)c1nn([C@H]2CC[C@H](N3CCN(C4CC(c5ccc(N[C@H]6CCC(=O)NC6=O)cc5)C4)CC3)CC2)cc1NC(=O)c1coc(-c2ccnc(NCC(F)(F)F)c2)n1]

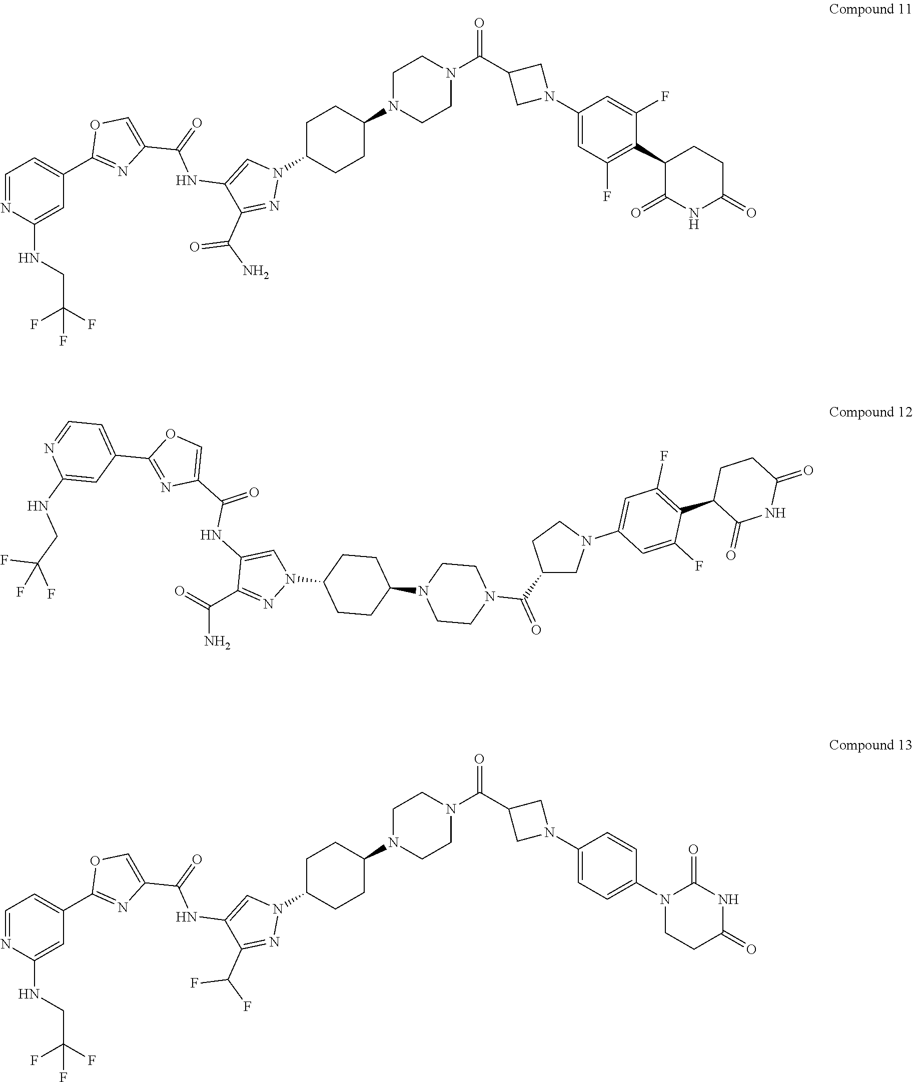 [SMILES: NC(=O)c1nn([C@H]2CC[C@H](N3CCN(C(=O)C4CN(c5cc(F)c([C@H]6CCC(=O)NC6=O)c(F)c5)C4)CC3)CC2)cc1NC(=O)c1coc(-c2ccnc(NCC(F)(F)F)c2)n1 ; NC(=O)c1nn([C@H]2CC[C@H](N3CCN(C(=O)[C@@H]4CCN(c5cc(F)c([C@H]6CCC(=O)NC6=O)c(F)c5)C4)CC3)CC2)cc1NC(=O)c1coc(-c2ccnc(NCC(F)(F)F)c2)n1 ; O=C1CCN(c2ccc(N3CC(C(=O)N4CCN([C@H]5CC[C@H](n6cc(NC(=O)c7coc(-c8ccnc(NCC(F)(F)F)c8)n7)c(C(F)F)n6)CC5)CC4)C3)cc2)C(=O)N1]

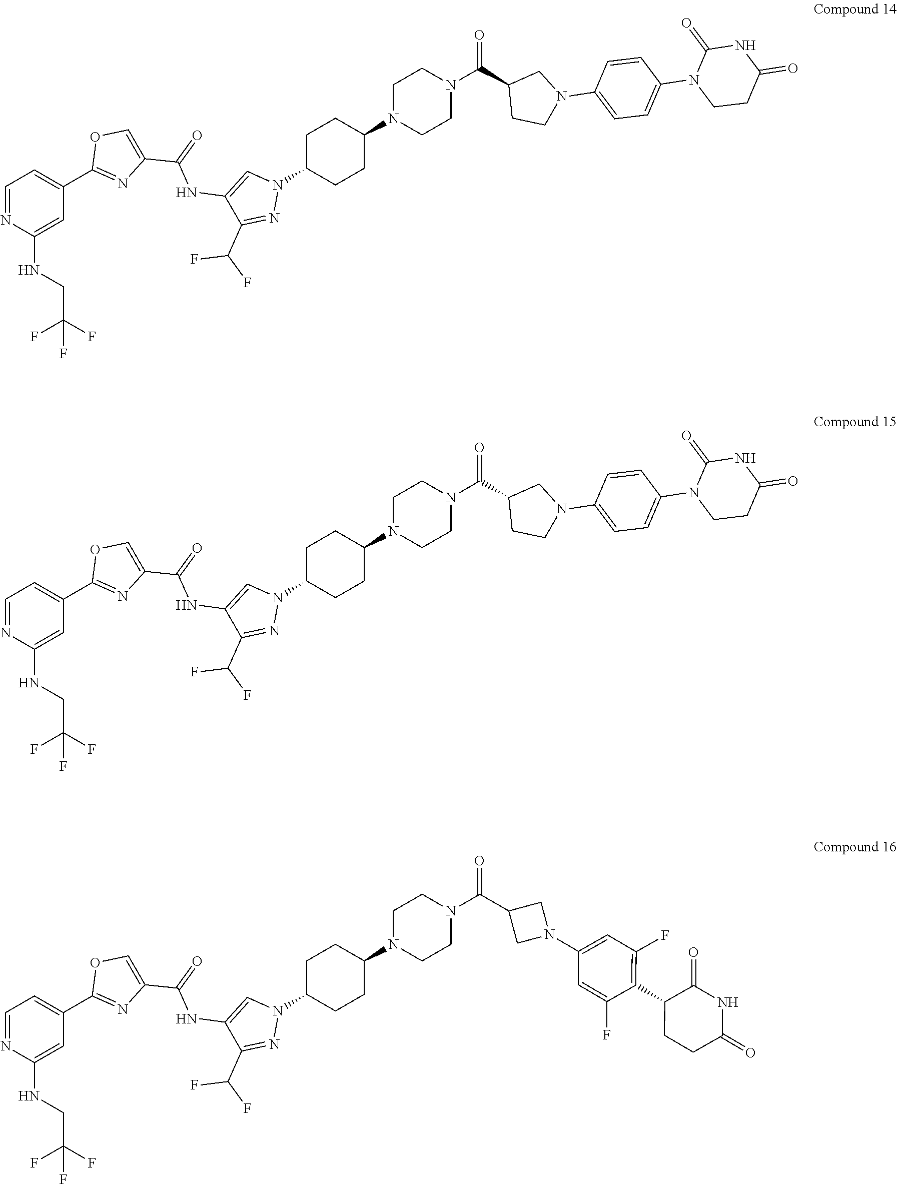 [SMILES: O=C1CCN(c2ccc(N3CC[C@@H](C(=O)N4CCN([C@H]5CC[C@H](n6cc(NC(=O)c7coc(-c8ccnc(NCC(F)(F)F)c8)n7)c(C(F)F)n6)CC5)CC4)C3)cc2)C(=O)N1 ; O=C1CCN(c2ccc(N3CC[C@H](C(=O)N4CCN([C@H]5CC[C@H](n6cc(NC(=O)c7coc(-c8ccnc(NCC(F)(F)F)c8)n7)c(C(F)F)n6)CC5)CC4)C3)cc2)C(=O)N1 ; O=C1CC[C@H](c2c(F)cc(N3CC(C(=O)N4CCN([C@H]5CC[C@H](n6cc(NC(=O)c7coc(-c8ccnc(NCC(F)(F)F)c8)n7)c(C(F)F)n6)CC5)CC4)C3)cc2F)C(=O)N1]

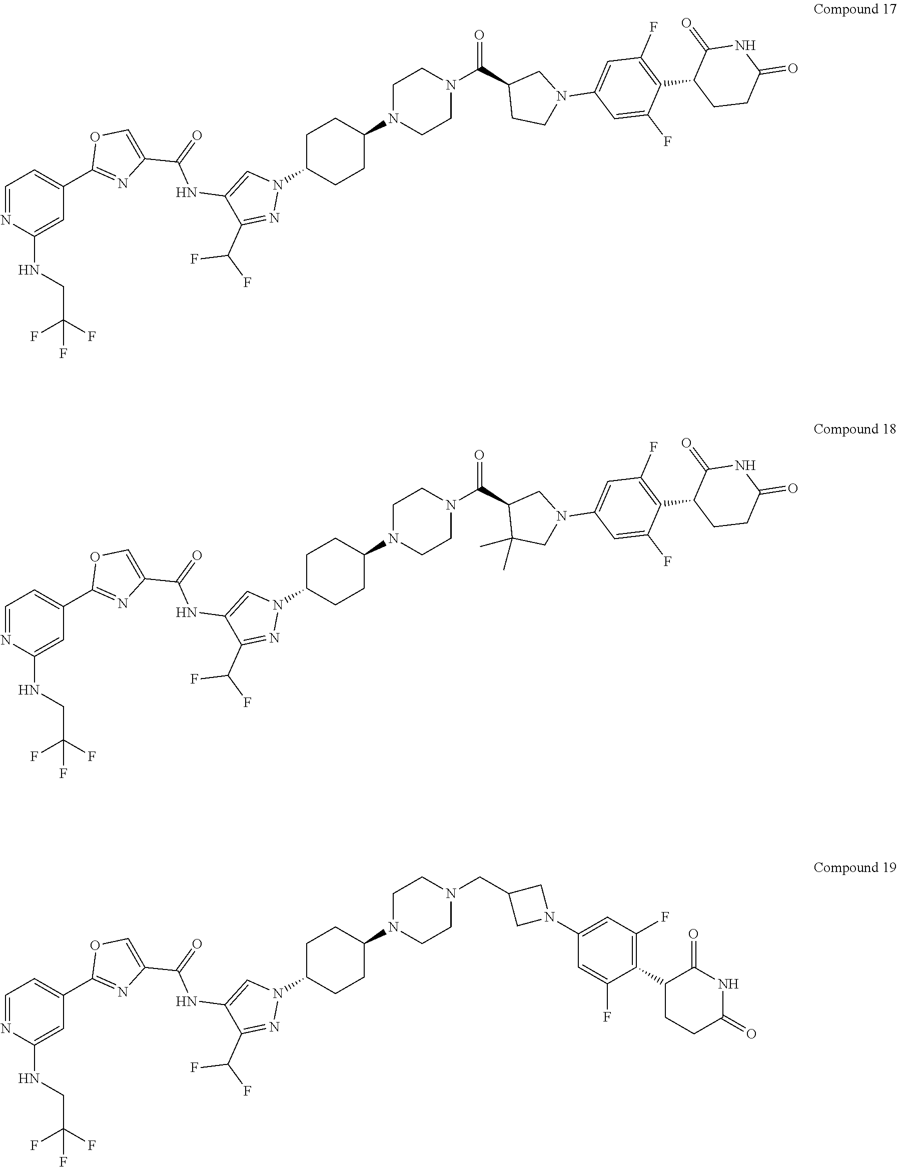

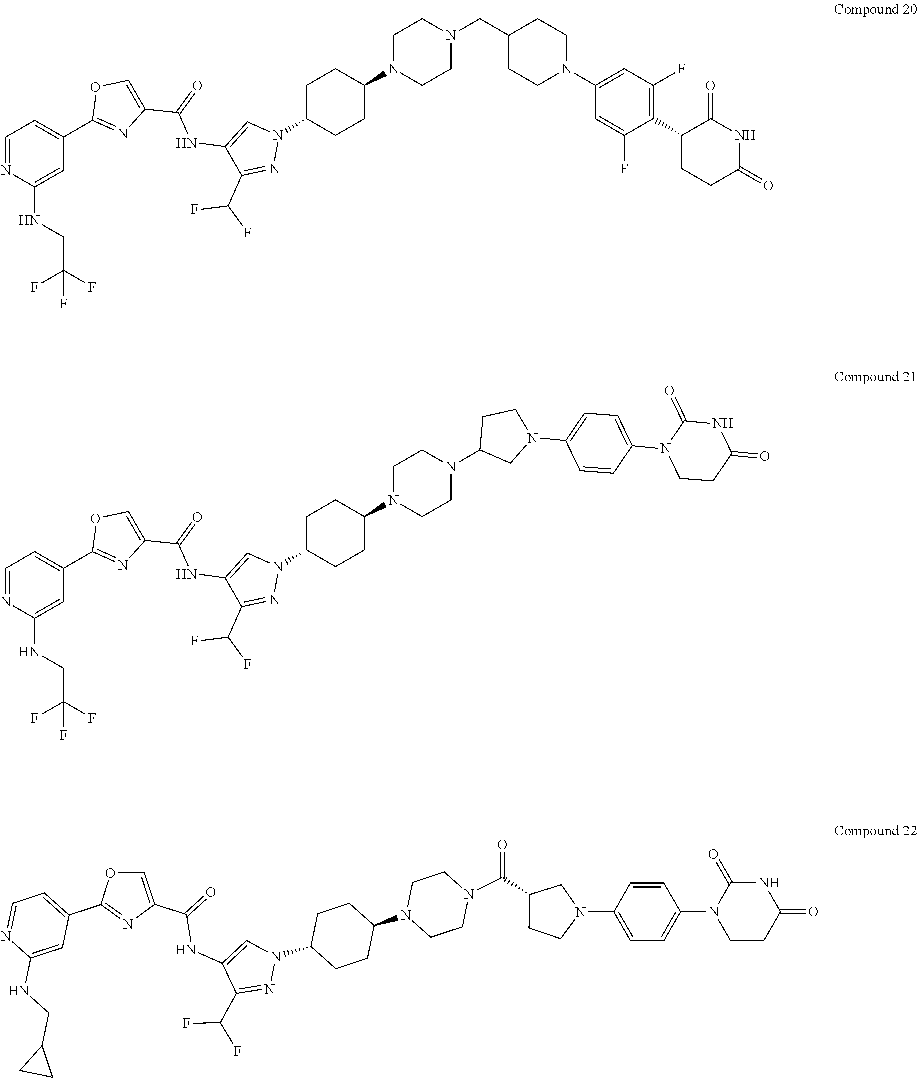 [SMILES: O=C1CC[C@H](c2c(F)cc(N3CCC(CN4CCN([C@H]5CC[C@H](n6cc(NC(=O)c7coc(-c8ccnc(NCC(F)(F)F)c8)n7)c(C(F)F)n6)CC5)CC4)CC3)cc2F)C(=O)N1 ; O=C1CCN(c2ccc(N3CCC(N4CCN([C@H]5CC[C@H](n6cc(NC(=O)c7coc(-c8ccnc(NCC(F)(F)F)c8)n7)c(C(F)F)n6)CC5)CC4)C3)cc2)C(=O)N1 ; O=C1CCN(c2ccc(N3CC[C@H](C(=O)N4CCN([C@H]5CC[C@H](n6cc(NC(=O)c7coc(-c8ccnc(NCC9CC9)c8)n7)c(C(F)F)n6)CC5)CC4)C3)cc2)C(=O)N1]

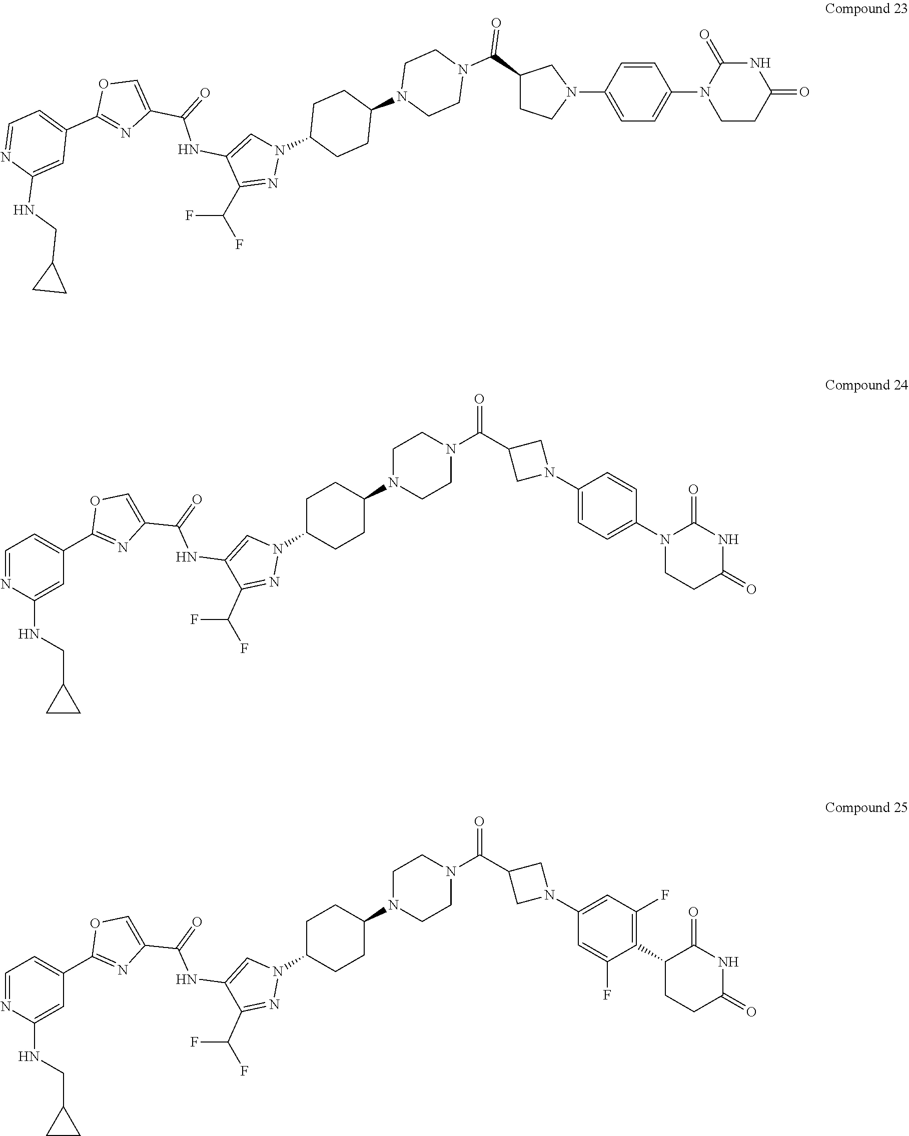 [SMILES: O=C1CCN(c2ccc(N3CC[C@@H](C(=O)N4CCN([C@H]5CC[C@H](n6cc(NC(=O)c7coc(-c8ccnc(NCC9CC9)c8)n7)c(C(F)F)n6)CC5)CC4)C3)cc2)C(=O)N1 ; O=C1CCN(c2ccc(N3CC(C(=O)N4CCN([C@H]5CC[C@H](n6cc(NC(=O)c7coc(-c8ccnc(NCC9CC9)c8)n7)c(C(F)F)n6)CC5)CC4)C3)cc2)C(=O)N1 ; O=C1CC[C@H](c2c(F)cc(N3CC(C(=O)N4CCN([C@H]5CC[C@H](n6cc(NC(=O)c7coc(-c8ccnc(NCC9CC9)c8)n7)c(C(F)F)n6)CC5)CC4)C3)cc2F)C(=O)N1]

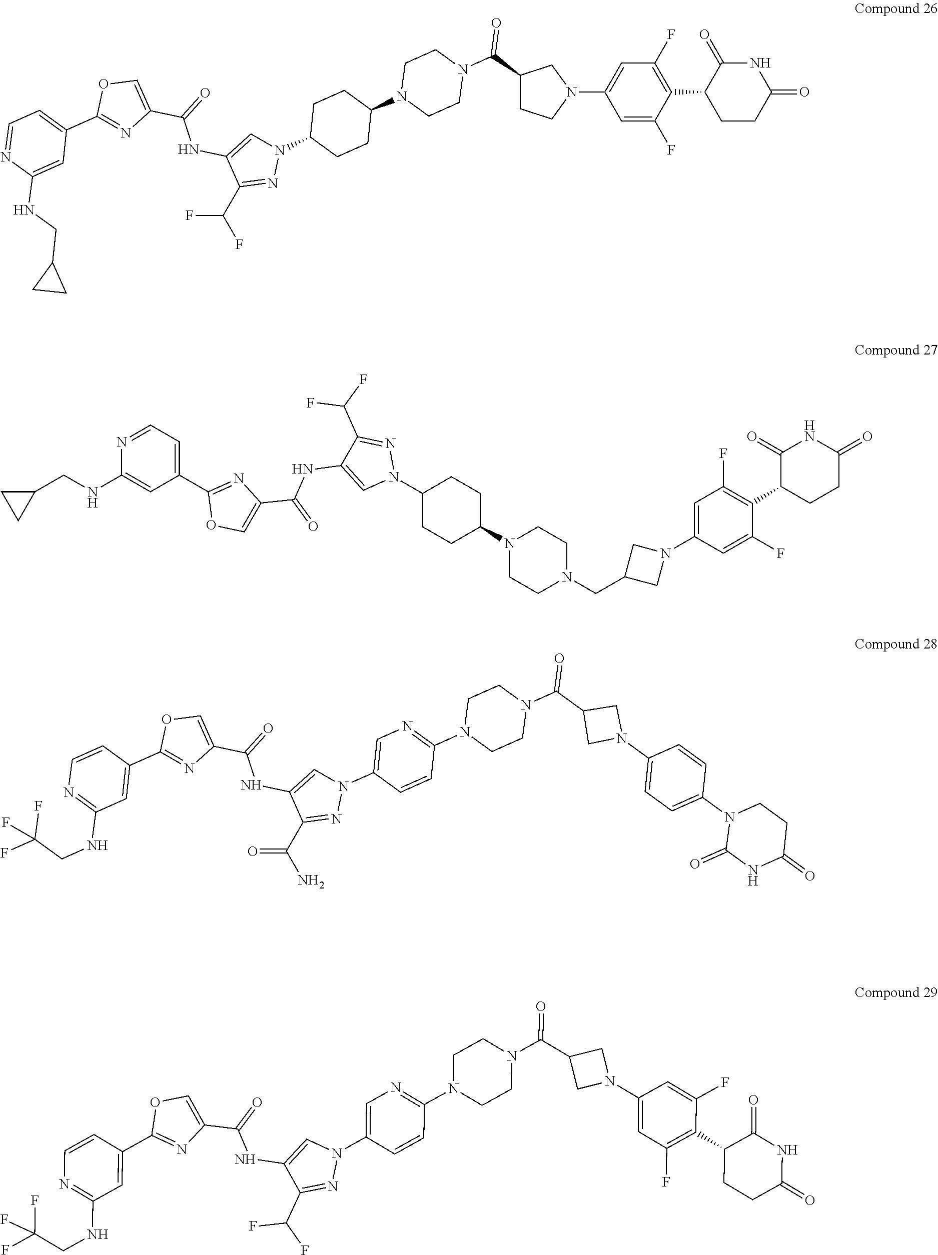

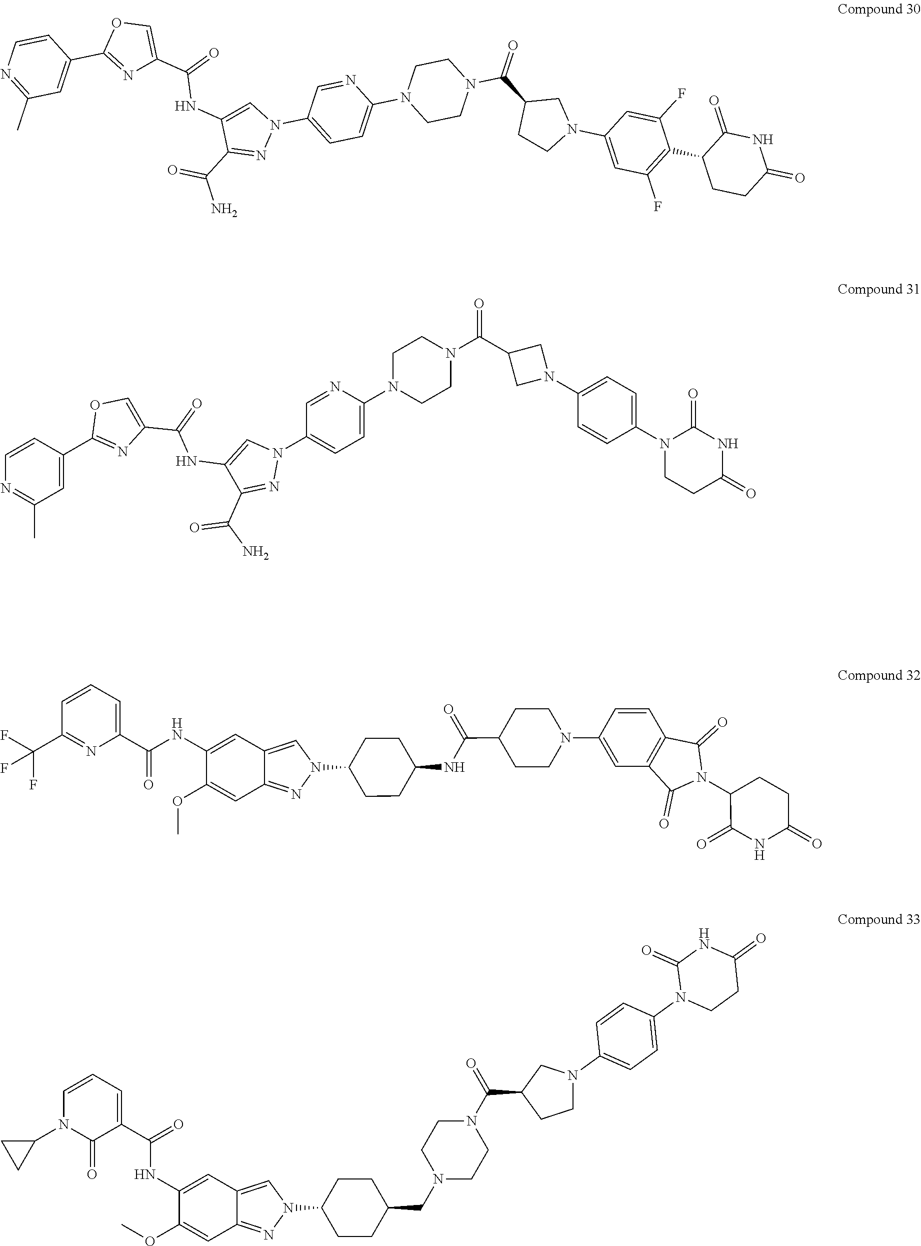 [SMILES: Cc1cc(-c2nc(C(=O)Nc3cn(-c4ccc(N5CCN(C(=O)[C@@H]6CCN(c7cc(F)c([C@H]8CCC(=O)NC8=O)c(F)c7)C6)CC5)nc4)nc3C(N)=O)co2)ccn1 ; Cc1cc(-c2nc(C(=O)Nc3cn(-c4ccc(N5CCN(C(=O)C6CN(c7ccc(N8CCC(=O)NC8=O)cc7)C6)CC5)nc4)nc3C(N)=O)co2)ccn1 ; COc1cc2nn([C@H]3CC[C@H](NC(=O)C4CCN(c5ccc6c(c5)C(=O)N(C5CCC(=O)NC5=O)C6=O)CC4)CC3)cc2cc1NC(=O)c1cccc(C(F)(F)F)n1 ; COc1cc2nn([C@H]3CC[C@H](CN4CCN(C(=O)[C@@H]5CCN(c6ccc(N7CCC(=O)NC7=O)cc6)C5)CC4)CC3)cc2cc1NC(=O)c1cccn(C2CC2)c1=O]

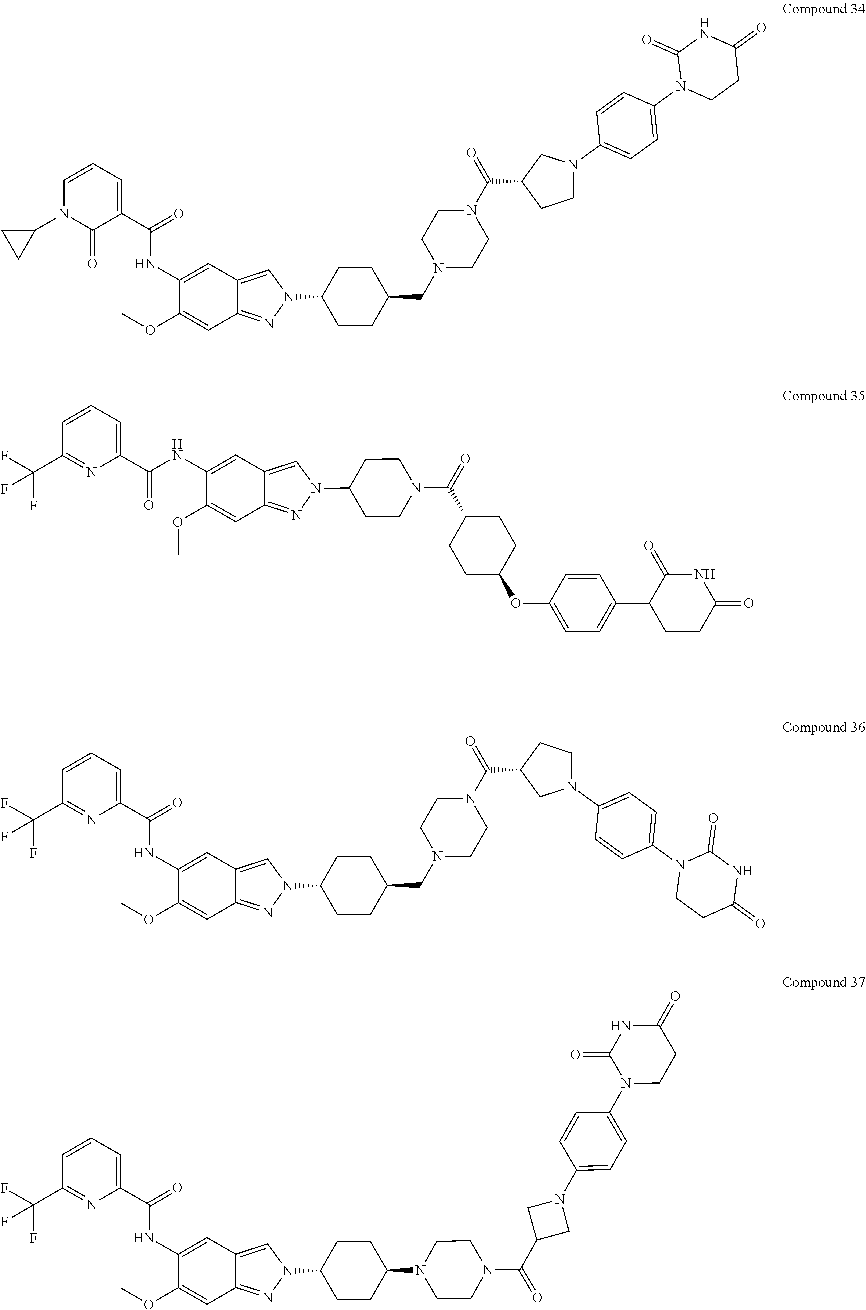 [SMILES: COc1cc2nn([C@H]3CC[C@H](CN4CCN(C(=O)[C@H]5CCN(c6ccc(N7CCC(=O)NC7=O)cc6)C5)CC4)CC3)cc2cc1NC(=O)c1cccn(C2CC2)c1=O ; COc1cc2nn(C3CCN(C(=O)[C@H]4CC[C@H](Oc5ccc(C6CCC(=O)NC6=O)cc5)CC4)CC3)cc2cc1NC(=O)c1cccc(C(F)(F)F)n1 ; COc1cc2nn([C@H]3CC[C@H](CN4CCN(C(=O)[C@@H]5CCN(c6ccc(N7CCC(=O)NC7=O)cc6)C5)CC4)CC3)cc2cc1NC(=O)c1cccc(C(F)(F)F)n1 ; COc1cc2nn([C@H]3CC[C@H](N4CCN(C(=O)C5CN(c6ccc(N7CCC(=O)NC7=O)cc6)C5)CC4)CC3)cc2cc1NC(=O)c1cccc(C(F)(F)F)n1]

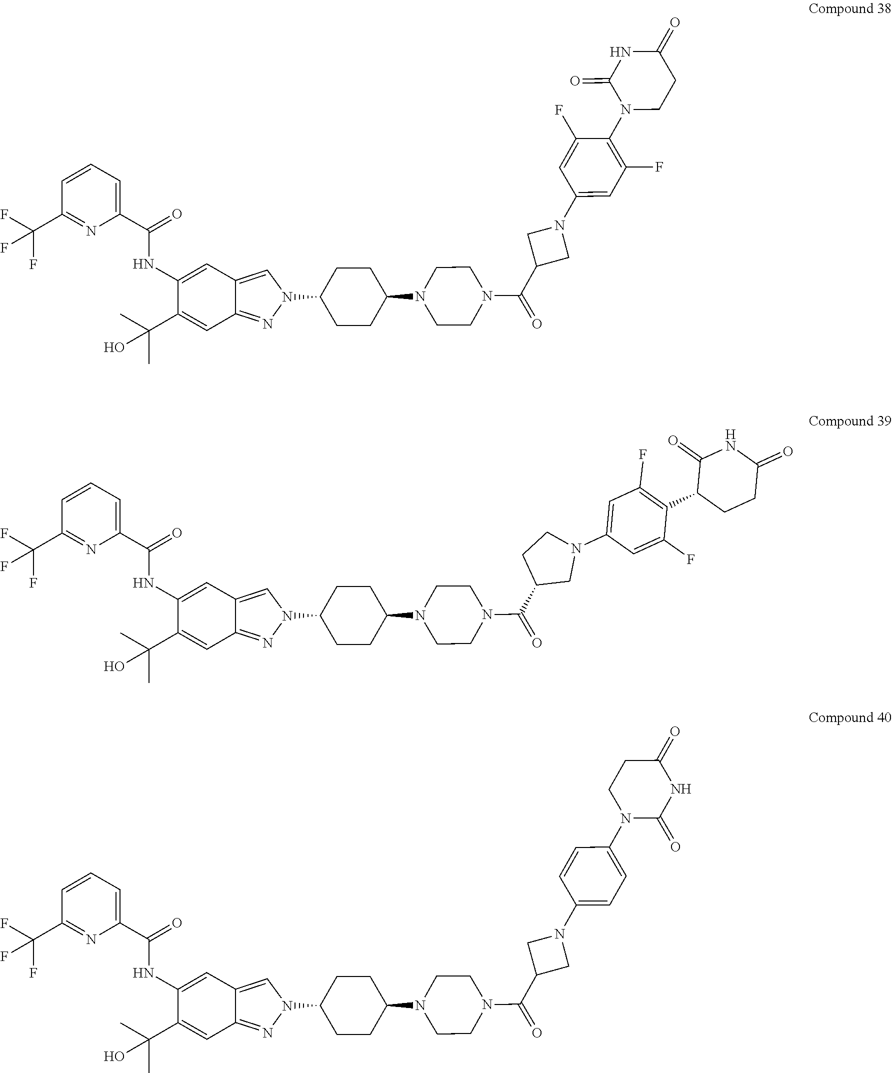 [SMILES: CC(C)(O)c1cc2nn([C@H]3CC[C@H](N4CCN(C(=O)C5CN(c6cc(F)c(N7CCC(=O)NC7=O)c(F)c6)C5)CC4)CC3)cc2cc1NC(=O)c1cccc(C(F)(F)F)n1 ; CC(C)(O)c1cc2nn([C@H]3CC[C@H](N4CCN(C(=O)[C@@H]5CCN(c6cc(F)c([C@H]7CCC(=O)NC7=O)c(F)c6)C5)CC4)CC3)cc2cc1NC(=O)c1cccc(C(F)(F)F)n1 ; CC(C)(O)c1cc2nn([C@H]3CC[C@H](N4CCN(C(=O)C5CN(c6ccc(N7CCC(=O)NC7=O)cc6)C5)CC4)CC3)cc2cc1NC(=O)c1cccc(C(F)(F)F)n1]

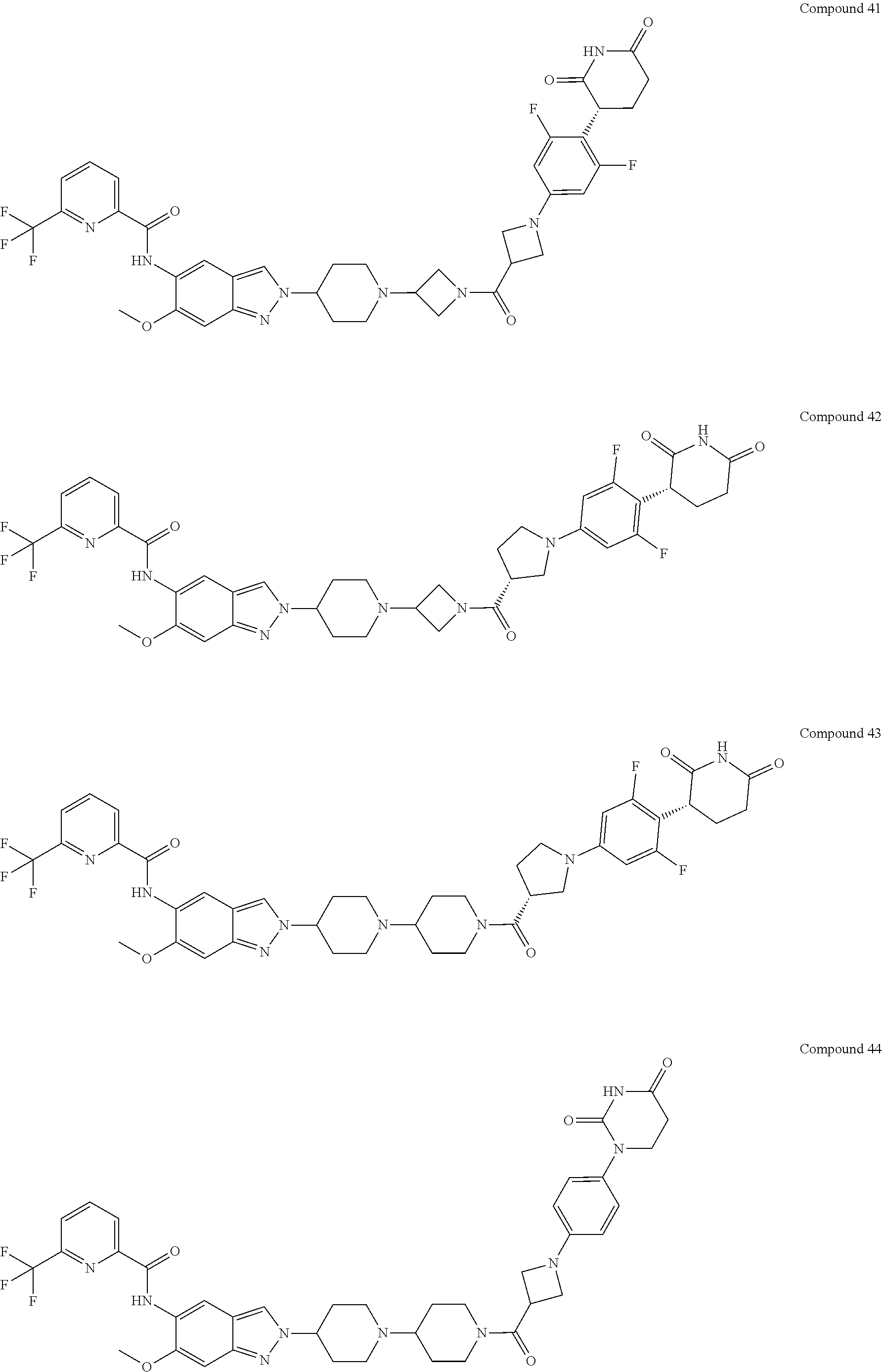 [SMILES: COc1cc2nn(C3CCN(C4CN(C(=O)C5CN(c6cc(F)c([C@H]7CCC(=O)NC7=O)c(F)c6)C5)C4)CC3)cc2cc1NC(=O)c1cccc(C(F)(F)F)n1 ; COc1cc2nn(C3CCN(C4CN(C(=O)[C@@H]5CCN(c6cc(F)c([C@H]7CCC(=O)NC7=O)c(F)c6)C5)C4)CC3)cc2cc1NC(=O)c1cccc(C(F)(F)F)n1 ; COc1cc2nn(C3CCN(C4CCN(C(=O)[C@@H]5CCN(c6cc(F)c([C@H]7CCC(=O)NC7=O)c(F)c6)C5)CC4)CC3)cc2cc1NC(=O)c1cccc(C(F)(F)F)n1 ; COc1cc2nn(C3CCN(C4CCN(C(=O)C5CN(c6ccc(N7CCC(=O)NC7=O)cc6)C5)CC4)CC3)cc2cc1NC(=O)c1cccc(C(F)(F)F)n1]

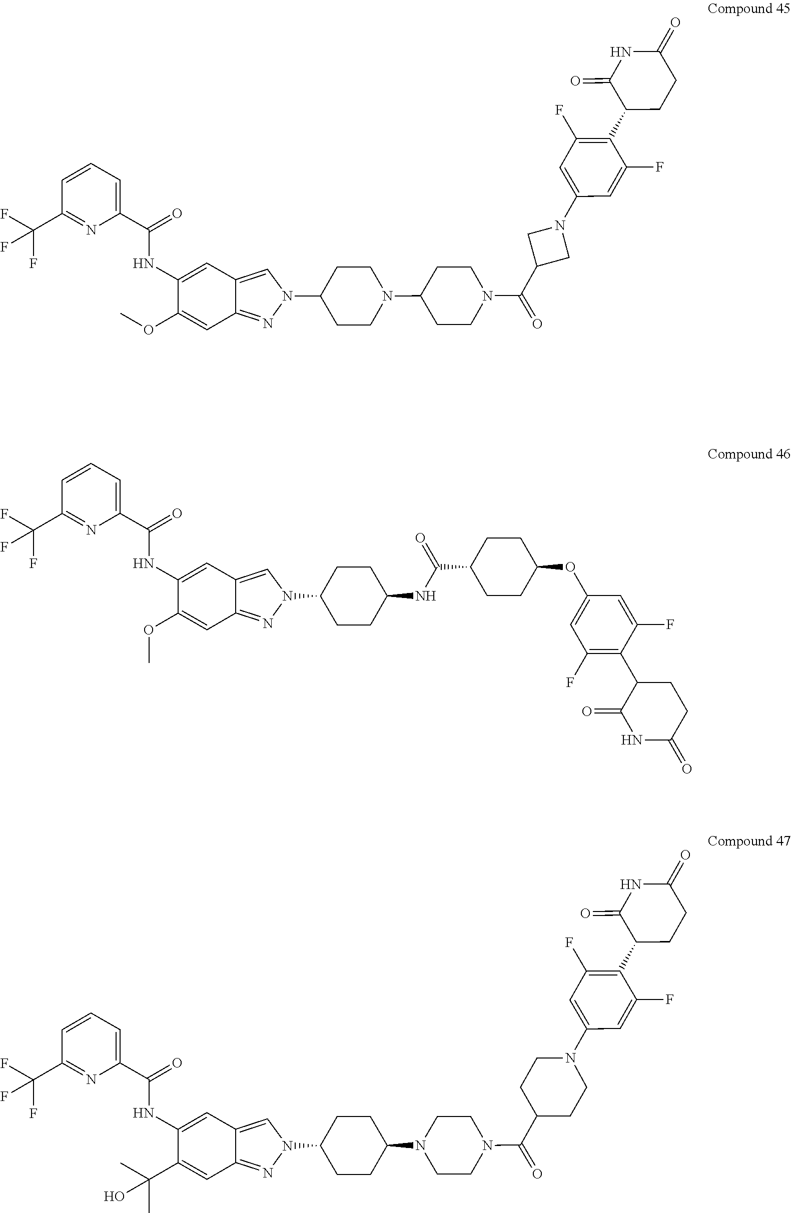 [SMILES: COc1cc2nn(C3CCN(C4CCN(C(=O)C5CN(c6cc(F)c([C@H]7CCC(=O)NC7=O)c(F)c6)C5)CC4)CC3)cc2cc1NC(=O)c1cccc(C(F)(F)F)n1 ; COc1cc2nn([C@H]3CC[C@H](NC(=O)[C@H]4CC[C@H](Oc5cc(F)c(C6CCC(=O)NC6=O)c(F)c5)CC4)CC3)cc2cc1NC(=O)c1cccc(C(F)(F)F)n1 ; CC(C)(O)c1cc2nn([C@H]3CC[C@H](N4CCN(C(=O)C5CCN(c6cc(F)c([C@H]7CCC(=O)NC7=O)c(F)c6)CC5)CC4)CC3)cc2cc1NC(=O)c1cccc(C(F)(F)F)n1]

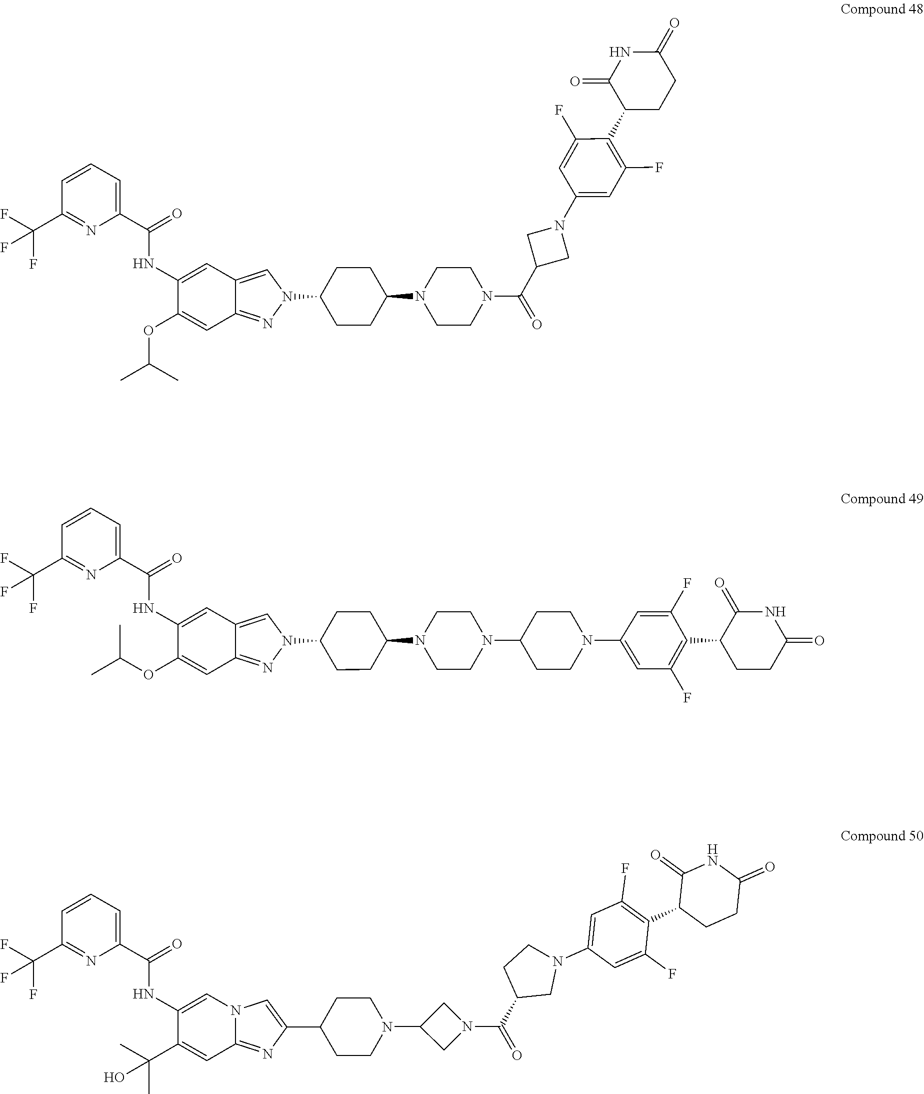 [SMILES: CC(C)Oc1cc2nn([C@H]3CC[C@H](N4CCN(C(=O)C5CN(c6cc(F)c([C@H]7CCC(=O)NC7=O)c(F)c6)C5)CC4)CC3)cc2cc1NC(=O)c1cccc(C(F)(F)F)n1 ; CC(C)Oc1cc2nn([C@H]3CC[C@H](N4CCN(C5CCN(c6cc(F)c([C@H]7CCC(=O)NC7=O)c(F)c6)CC5)CC4)CC3)cc2cc1NC(=O)c1cccc(C(F)(F)F)n1 ; CC(C)(O)c1cc2nc(C3CCN(C4CN(C(=O)[C@@H]5CCN(c6cc(F)c([C@H]7CCC(=O)NC7=O)c(F)c6)C5)C4)CC3)cn2cc1NC(=O)c1cccc(C(F)(F)F)n1]

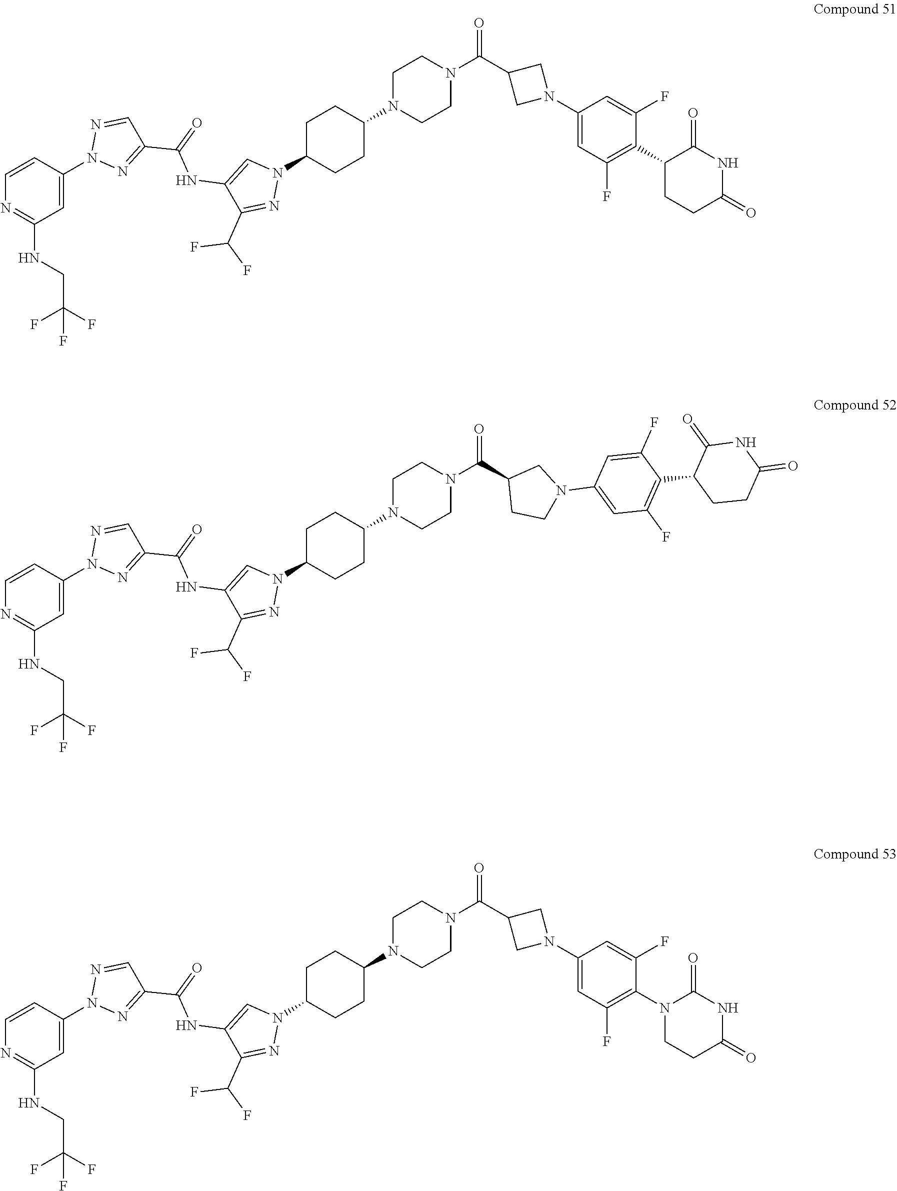 [SMILES: O=C1CC[C@H](c2c(F)cc(N3CC(C(=O)N4CCN([C@H]5CC[C@H](n6cc(NC(=O)c7cnn(-c8ccnc(NCC(F)(F)F)c8)n7)c(C(F)F)n6)CC5)CC4)C3)cc2F)C(=O)N1 ; O=C1CC[C@H](c2c(F)cc(N3CC[C@@H](C(=O)N4CCN([C@H]5CC[C@H](n6cc(NC(=O)c7cnn(-c8ccnc(NCC(F)(F)F)c8)n7)c(C(F)F)n6)CC5)CC4)C3)cc2F)C(=O)N1 ; O=C1CCN(c2c(F)cc(N3CC(C(=O)N4CCN([C@H]5CC[C@H](n6cc(NC(=O)c7cnn(-c8ccnc(NCC(F)(F)F)c8)n7)c(C(F)F)n6)CC5)CC4)C3)cc2F)C(=O)N1]

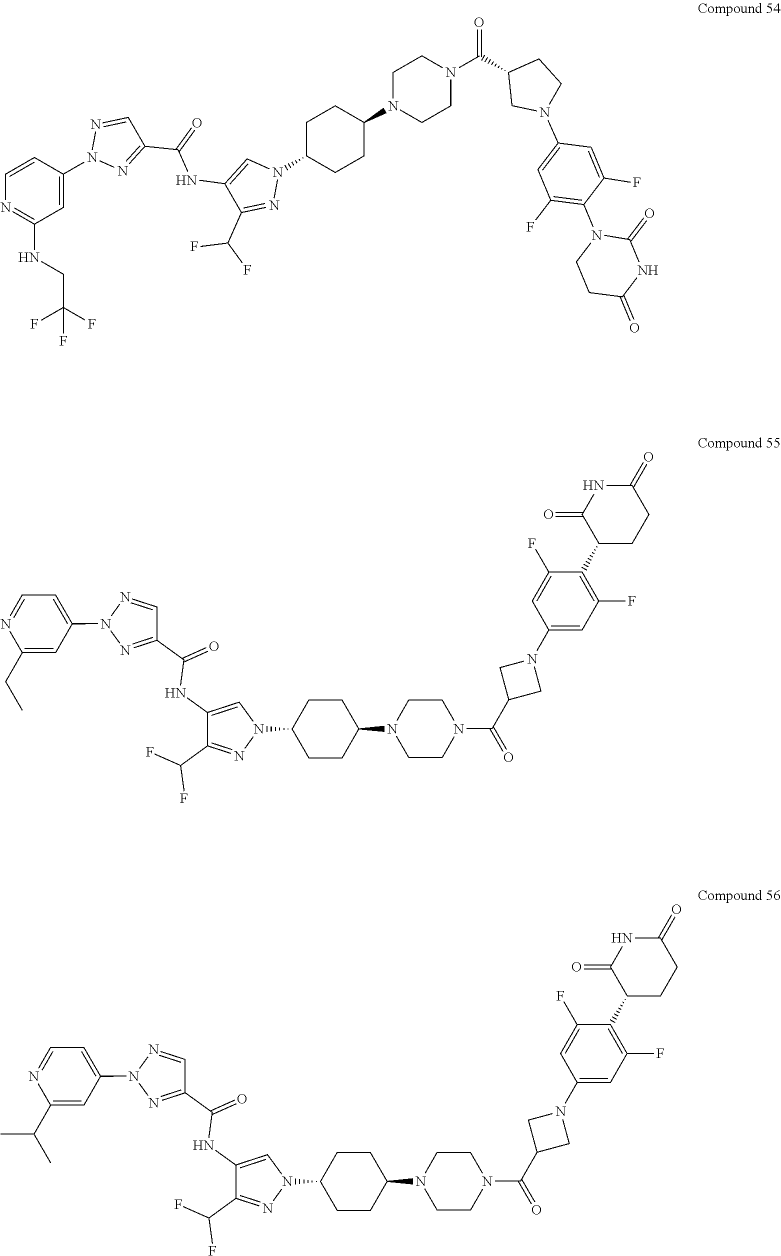 [SMILES: O=C1CCN(c2c(F)cc(N3CC[C@@H](C(=O)N4CCN([C@H]5CC[C@H](n6cc(NC(=O)c7cnn(-c8ccnc(NCC(F)(F)F)c8)n7)c(C(F)F)n6)CC5)CC4)C3)cc2F)C(=O)N1 ; CCc1cc(-n2ncc(C(=O)Nc3cn([C@H]4CC[C@H](N5CCN(C(=O)C6CN(c7cc(F)c([C@H]8CCC(=O)NC8=O)c(F)c7)C6)CC5)CC4)nc3C(F)F)n2)ccn1 ; CC(C)c1cc(-n2ncc(C(=O)Nc3cn([C@H]4CC[C@H](N5CCN(C(=O)C6CN(c7cc(F)c([C@H]8CCC(=O)NC8=O)c(F)c7)C6)CC5)CC4)nc3C(F)F)n2)ccn1]

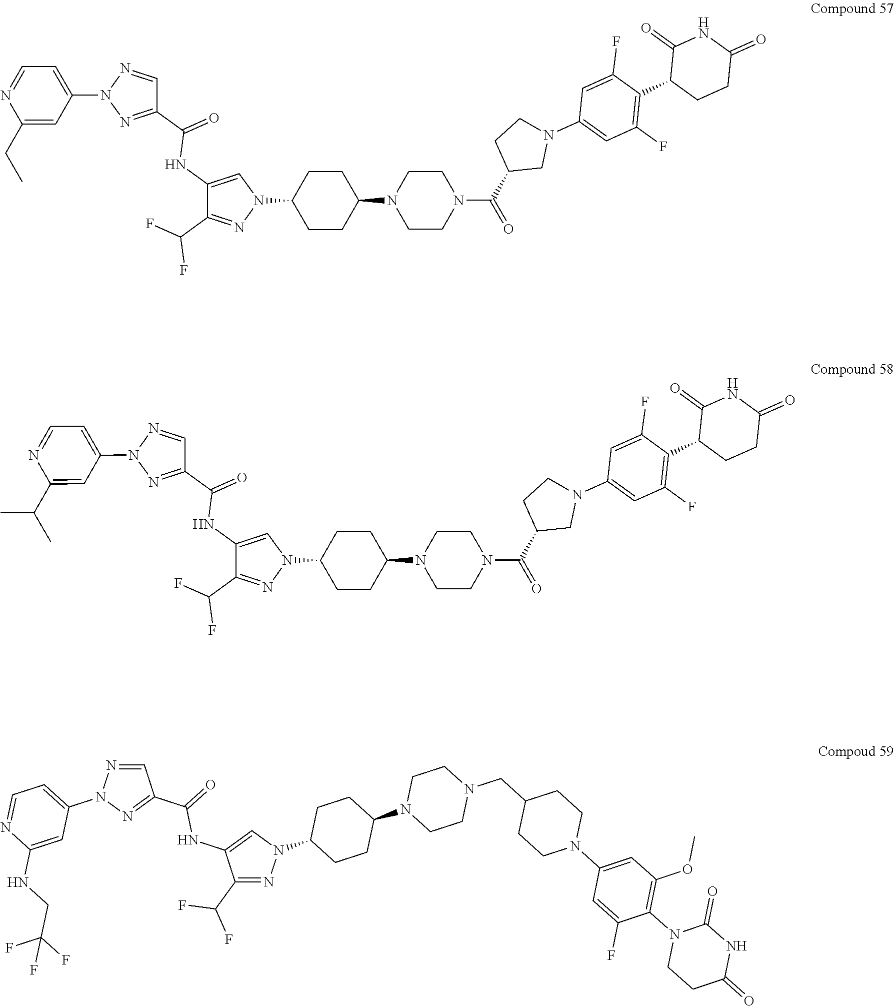 [SMILES: CCc1cc(-n2ncc(C(=O)Nc3cn([C@H]4CC[C@H](N5CCN(C(=O)[C@@H]6CCN(c7cc(F)c([C@H]8CCC(=O)NC8=O)c(F)c7)C6)CC5)CC4)nc3C(F)F)n2)ccn1 ; CC(C)c1cc(-n2ncc(C(=O)Nc3cn([C@H]4CC[C@H](N5CCN(C(=O)[C@@H]6CCN(c7cc(F)c([C@H]8CCC(=O)NC8=O)c(F)c7)C6)CC5)CC4)nc3C(F)F)n2)ccn1 ; COc1cc(N2CCC(CN3CCN([C@H]4CC[C@H](n5cc(NC(=O)c6cnn(-c7ccnc(NCC(F)(F)F)c7)n6)c(C(F)F)n5)CC4)CC3)CC2)cc(F)c1N1CCC(=O)NC1=O]

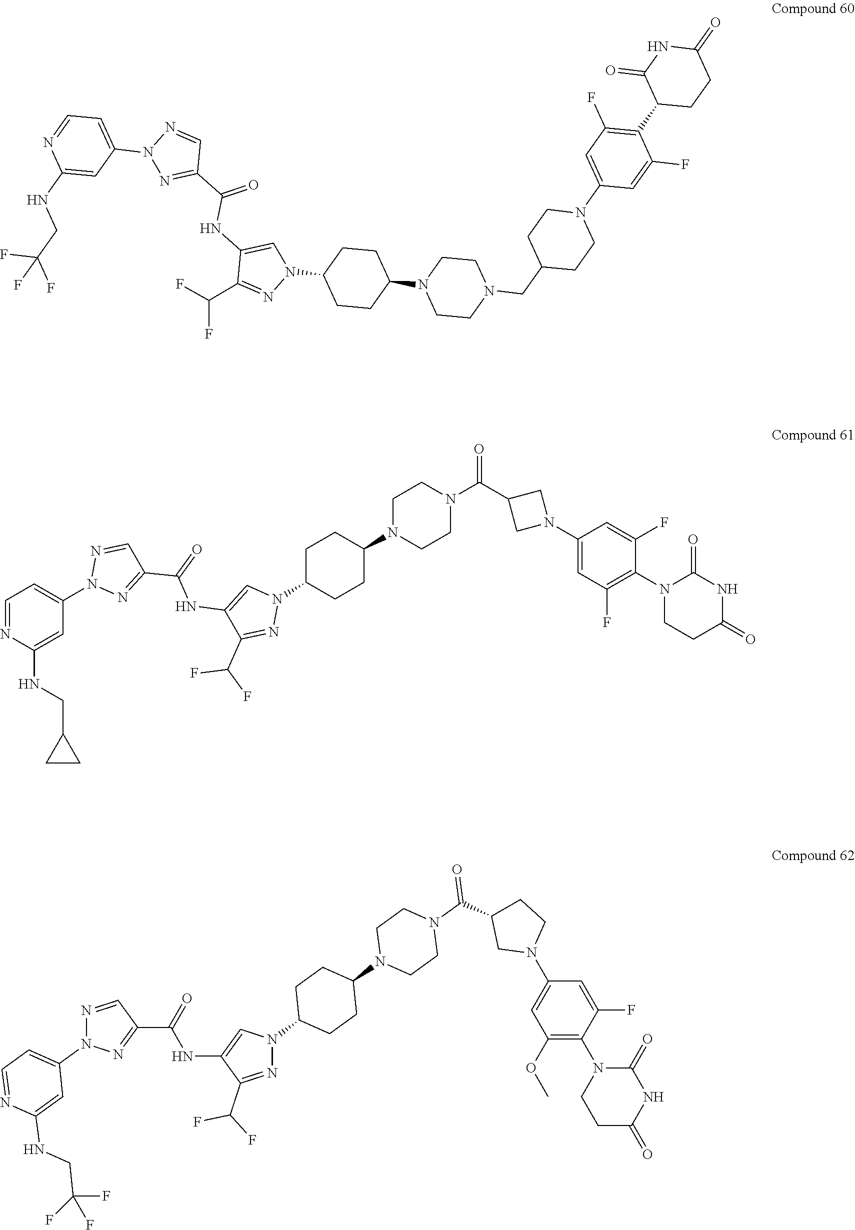 [SMILES: O=C1CC[C@H](c2c(F)cc(N3CCC(CN4CCN([C@H]5CC[C@H](n6cc(NC(=O)c7cnn(-c8ccnc(NCC(F)(F)F)c8)n7)c(C(F)F)n6)CC5)CC4)CC3)cc2F)C(=O)N1 ; O=C1CCN(c2c(F)cc(N3CC(C(=O)N4CCN([C@H]5CC[C@H](n6cc(NC(=O)c7cnn(-c8ccnc(NCC9CC9)c8)n7)c(C(F)F)n6)CC5)CC4)C3)cc2F)C(=O)N1 ; COc1cc(N2CC[C@@H](C(=O)N3CCN([C@H]4CC[C@H](n5cc(NC(=O)c6cnn(-c7ccnc(NCC(F)(F)F)c7)n6)c(C(F)F)n5)CC4)CC3)C2)cc(F)c1N1CCC(=O)NC1=O]

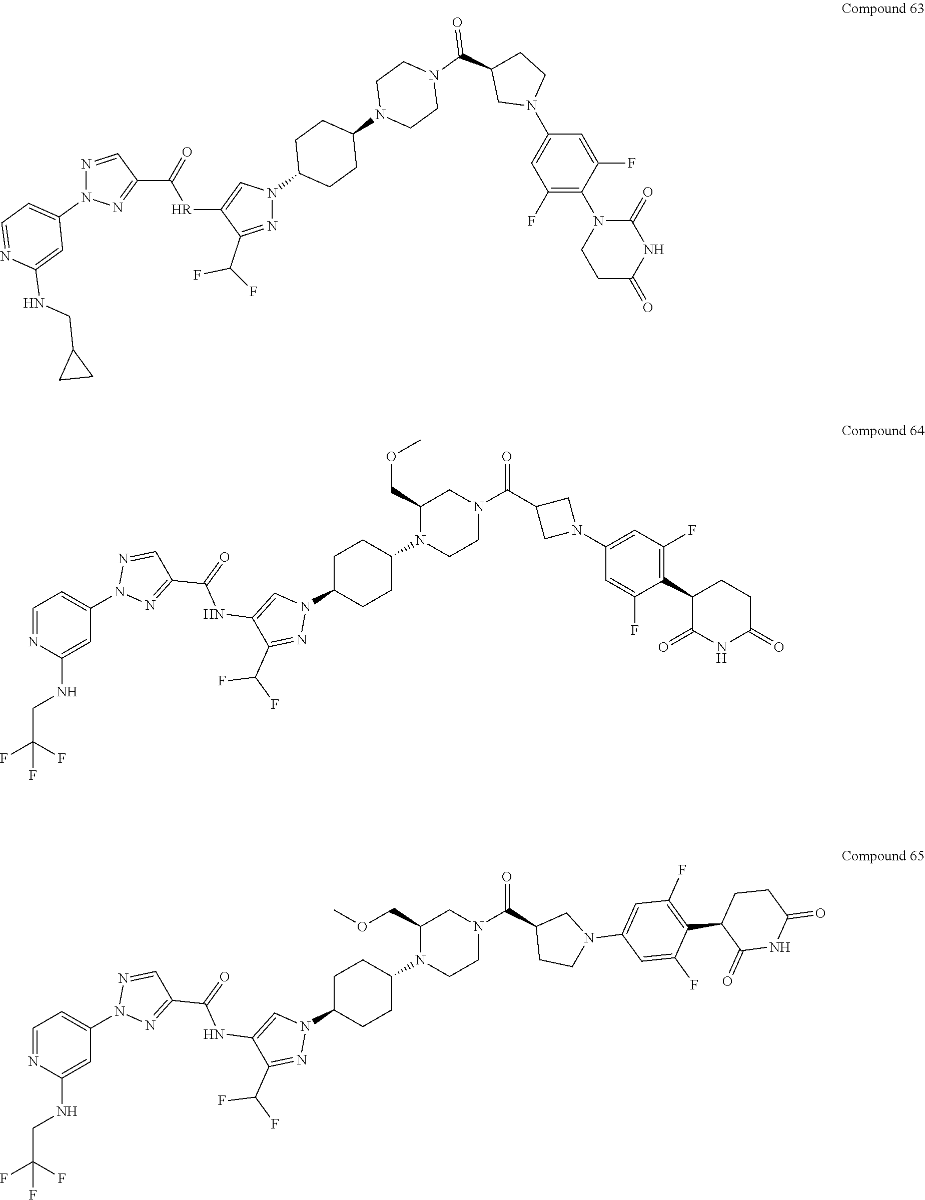

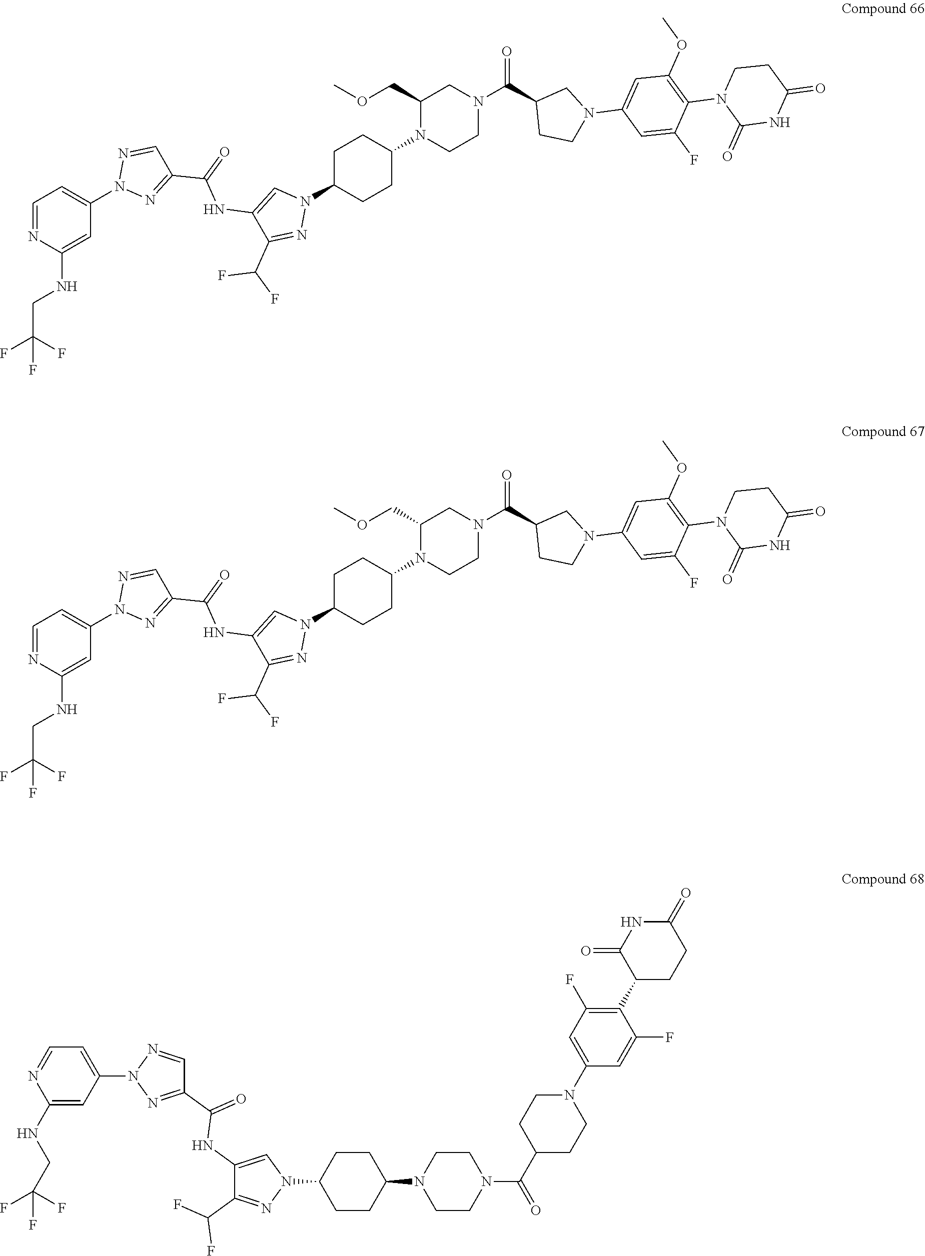 [SMILES: COC[C@H]1CN(C(=O)[C@@H]2CCN(c3cc(F)c(N4CCC(=O)NC4=O)c(OC)c3)C2)CCN1[C@H]1CC[C@H](n2cc(NC(=O)c3cnn(-c4ccnc(NCC(F)(F)F)c4)n3)c(C(F)F)n2)CC1 ; COC[C@@H]1CN(C(=O)[C@@H]2CCN(c3cc(F)c(N4CCC(=O)NC4=O)c(OC)c3)C2)CCN1[C@H]1CC[C@H](n2cc(NC(=O)c3cnn(-c4ccnc(NCC(F)(F)F)c4)n3)c(C(F)F)n2)CC1 ; O=C1CC[C@H](c2c(F)cc(N3CCC(C(=O)N4CCN([C@H]5CC[C@H](n6cc(NC(=O)c7cnn(-c8ccnc(NCC(F)(F)F)c8)n7)c(C(F)F)n6)CC5)CC4)CC3)cc2F)C(=O)N1]

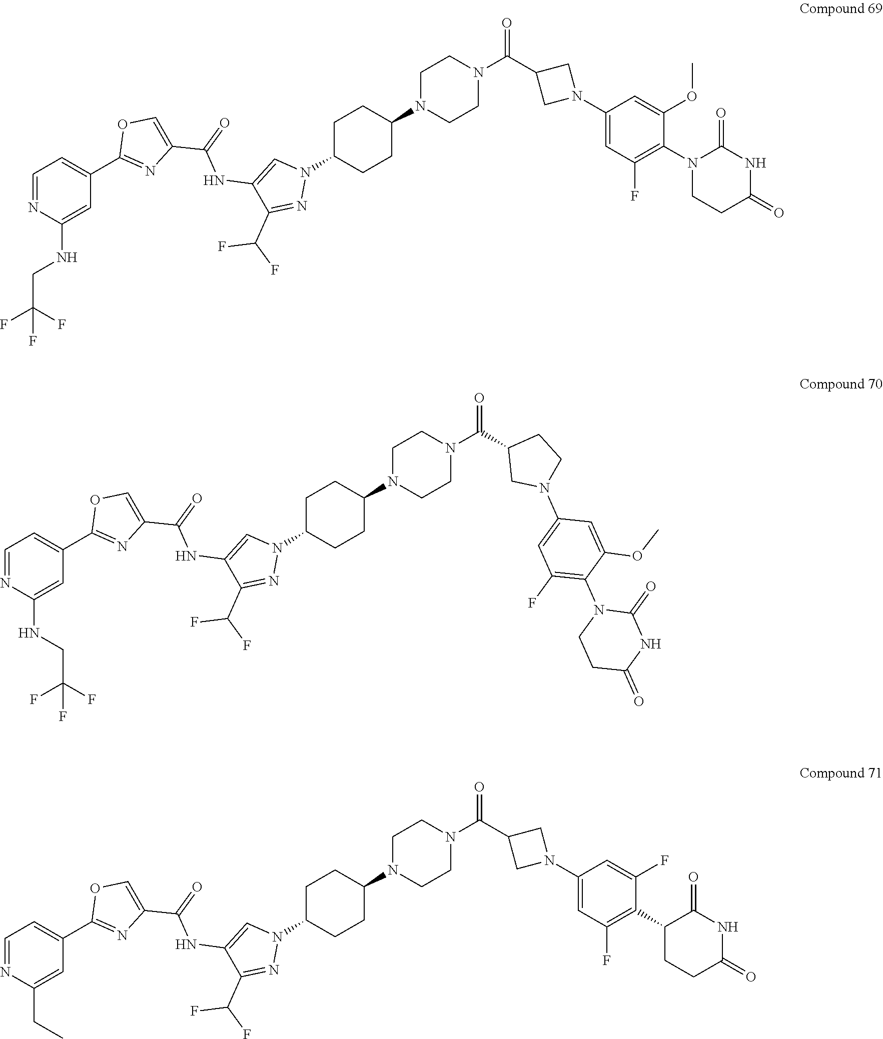 [SMILES: COc1cc(N2CC(C(=O)N3CCN([C@H]4CC[C@H](n5cc(NC(=O)c6coc(-c7ccnc(NCC(F)(F)F)c7)n6)c(C(F)F)n5)CC4)CC3)C2)cc(F)c1N1CCC(=O)NC1=O ; COc1cc(N2CC[C@@H](C(=O)N3CCN([C@H]4CC[C@H](n5cc(NC(=O)c6coc(-c7ccnc(NCC(F)(F)F)c7)n6)c(C(F)F)n5)CC4)CC3)C2)cc(F)c1N1CCC(=O)NC1=O ; CCc1cc(-c2nc(C(=O)Nc3cn([C@H]4CC[C@H](N5CCN(C(=O)C6CN(c7cc(F)c([C@H]8CCC(=O)NC8=O)c(F)c7)C6)CC5)CC4)nc3C(F)F)co2)ccn1]

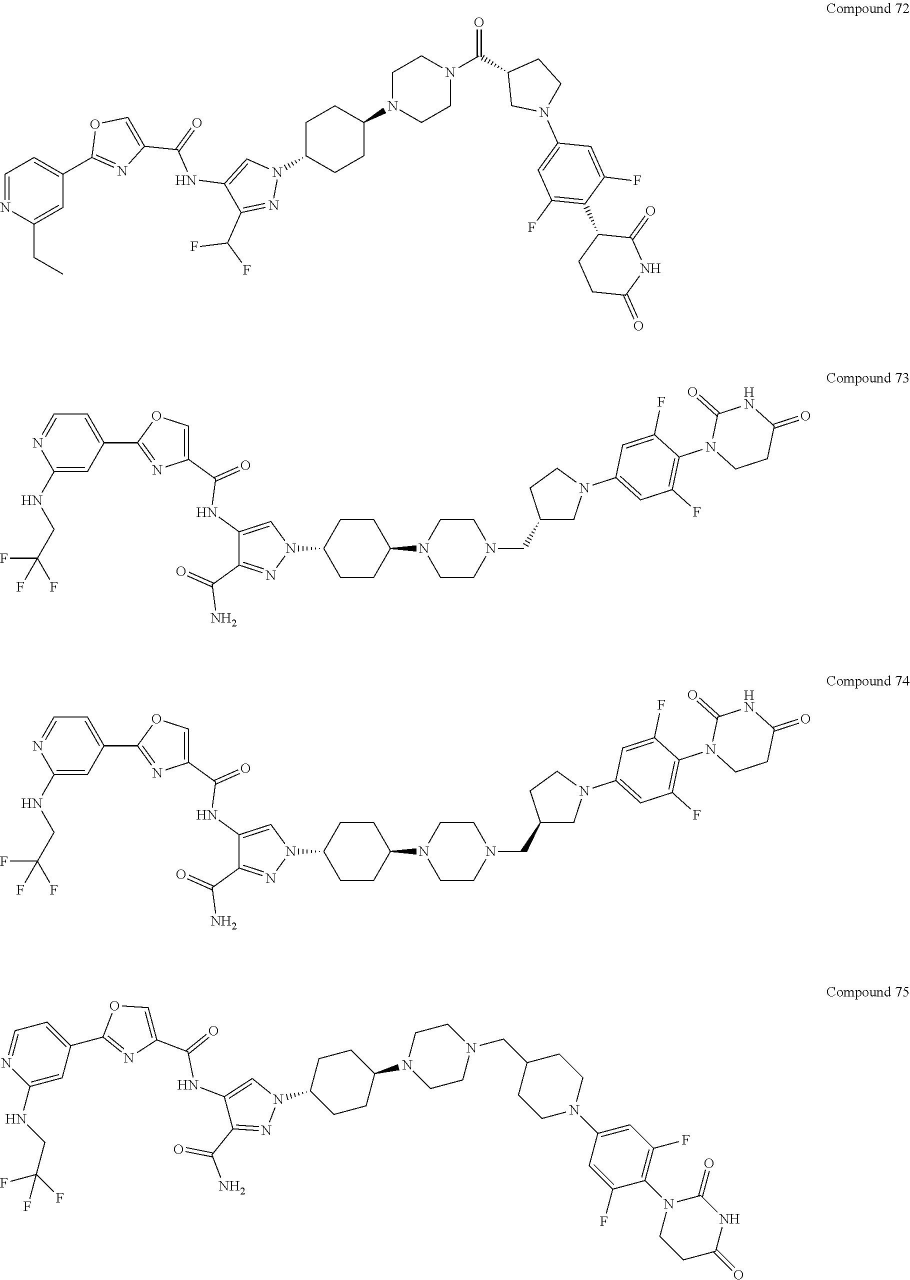 [SMILES: CCc1cc(-c2nc(C(=O)Nc3cn([C@H]4CC[C@H](N5CCN(C(=O)[C@@H]6CCN(c7cc(F)c([C@H]8CCC(=O)NC8=O)c(F)c7)C6)CC5)CC4)nc3C(F)F)co2)ccn1 ; NC(=O)c1nn([C@H]2CC[C@H](N3CCN(C[C@@H]4CCN(c5cc(F)c(N6CCC(=O)NC6=O)c(F)c5)C4)CC3)CC2)cc1NC(=O)c1coc(-c2ccnc(NCC(F)(F)F)c2)n1 ; NC(=O)c1nn([C@H]2CC[C@H](N3CCN(C[C@H]4CCN(c5cc(F)c(N6CCC(=O)NC6=O)c(F)c5)C4)CC3)CC2)cc1NC(=O)c1coc(-c2ccnc(NCC(F)(F)F)c2)n1 ; NC(=O)c1nn([C@H]2CC[C@H](N3CCN(CC4CCN(c5cc(F)c(N6CCC(=O)NC6=O)c(F)c5)CC4)CC3)CC2)cc1NC(=O)c1coc(-c2ccnc(NCC(F)(F)F)c2)n1]

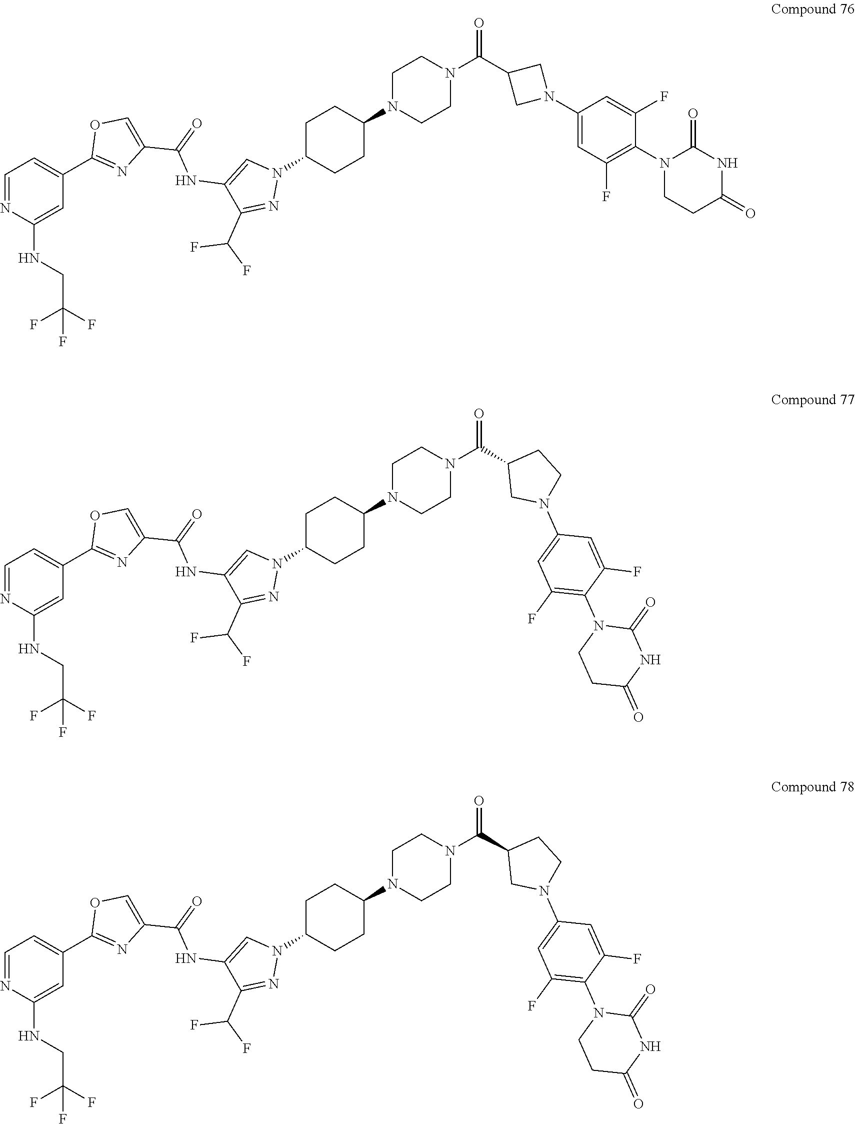 [SMILES: O=C1CCN(c2c(F)cc(N3CC(C(=O)N4CCN([C@H]5CC[C@H](n6cc(NC(=O)c7coc(-c8ccnc(NCC(F)(F)F)c8)n7)c(C(F)F)n6)CC5)CC4)C3)cc2F)C(=O)N1 ; O=C1CCN(c2c(F)cc(N3CC[C@@H](C(=O)N4CCN([C@H]5CC[C@H](n6cc(NC(=O)c7coc(-c8ccnc(NCC(F)(F)F)c8)n7)c(C(F)F)n6)CC5)CC4)C3)cc2F)C(=O)N1 ; O=C1CCN(c2c(F)cc(N3CC[C@H](C(=O)N4CCN([C@H]5CC[C@H](n6cc(NC(=O)c7coc(-c8ccnc(NCC(F)(F)F)c8)n7)c(C(F)F)n6)CC5)CC4)C3)cc2F)C(=O)N1]

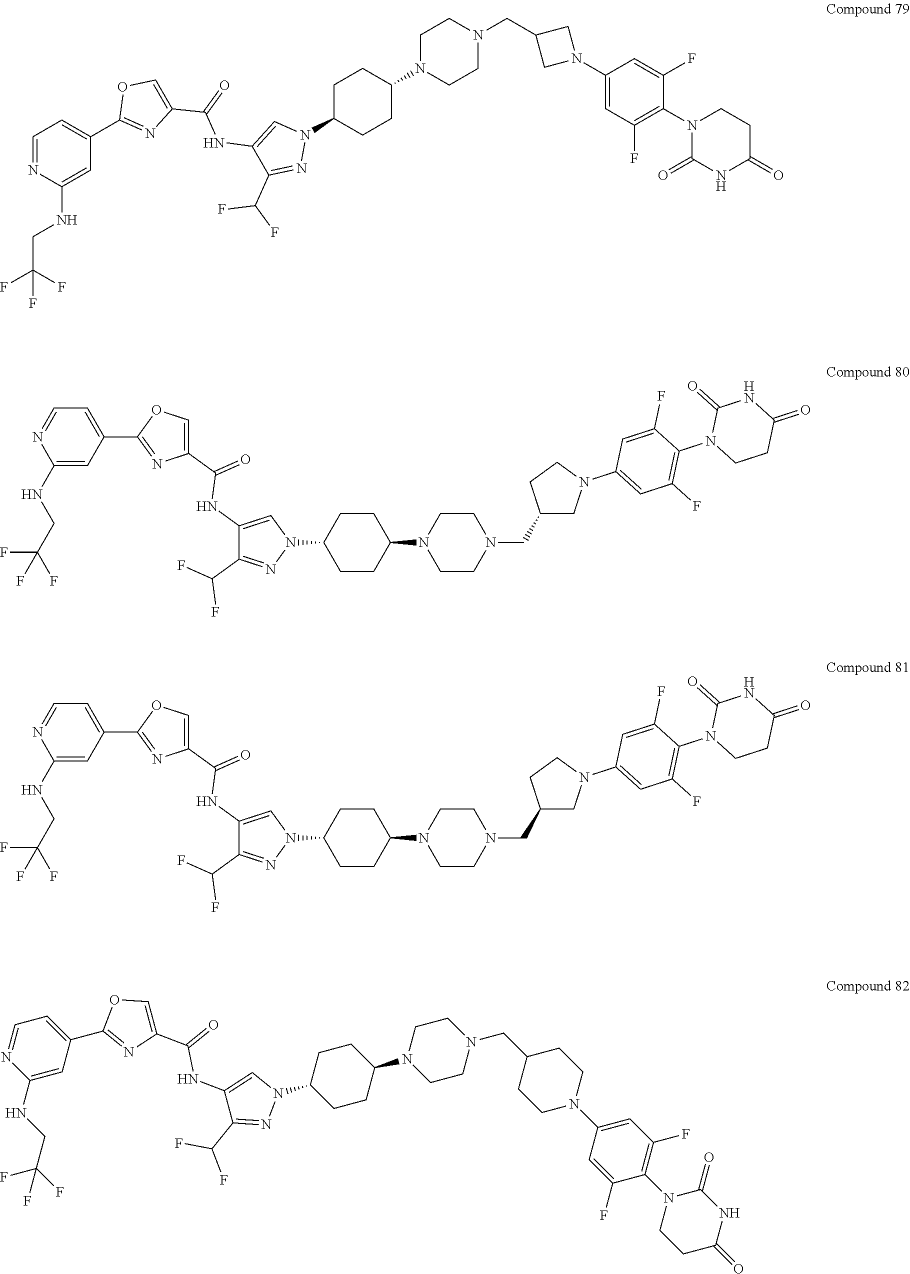 [SMILES: O=C1CCN(c2c(F)cc(N3CC(CN4CCN([C@H]5CC[C@H](n6cc(NC(=O)c7coc(-c8ccnc(NCC(F)(F)F)c8)n7)c(C(F)F)n6)CC5)CC4)C3)cc2F)C(=O)N1 ; O=C1CCN(c2c(F)cc(N3CC[C@@H](CN4CCN([C@H]5CC[C@H](n6cc(NC(=O)c7coc(-c8ccnc(NCC(F)(F)F)c8)n7)c(C(F)F)n6)CC5)CC4)C3)cc2F)C(=O)N1 ; O=C1CCN(c2c(F)cc(N3CC[C@H](CN4CCN([C@H]5CC[C@H](n6cc(NC(=O)c7coc(-c8ccnc(NCC(F)(F)F)c8)n7)c(C(F)F)n6)CC5)CC4)C3)cc2F)C(=O)N1 ; O=C1CCN(c2c(F)cc(N3CCC(CN4CCN([C@H]5CC[C@H](n6cc(NC(=O)c7coc(-c8ccnc(NCC(F)(F)F)c8)n7)c(C(F)F)n6)CC5)CC4)CC3)cc2F)C(=O)N1]

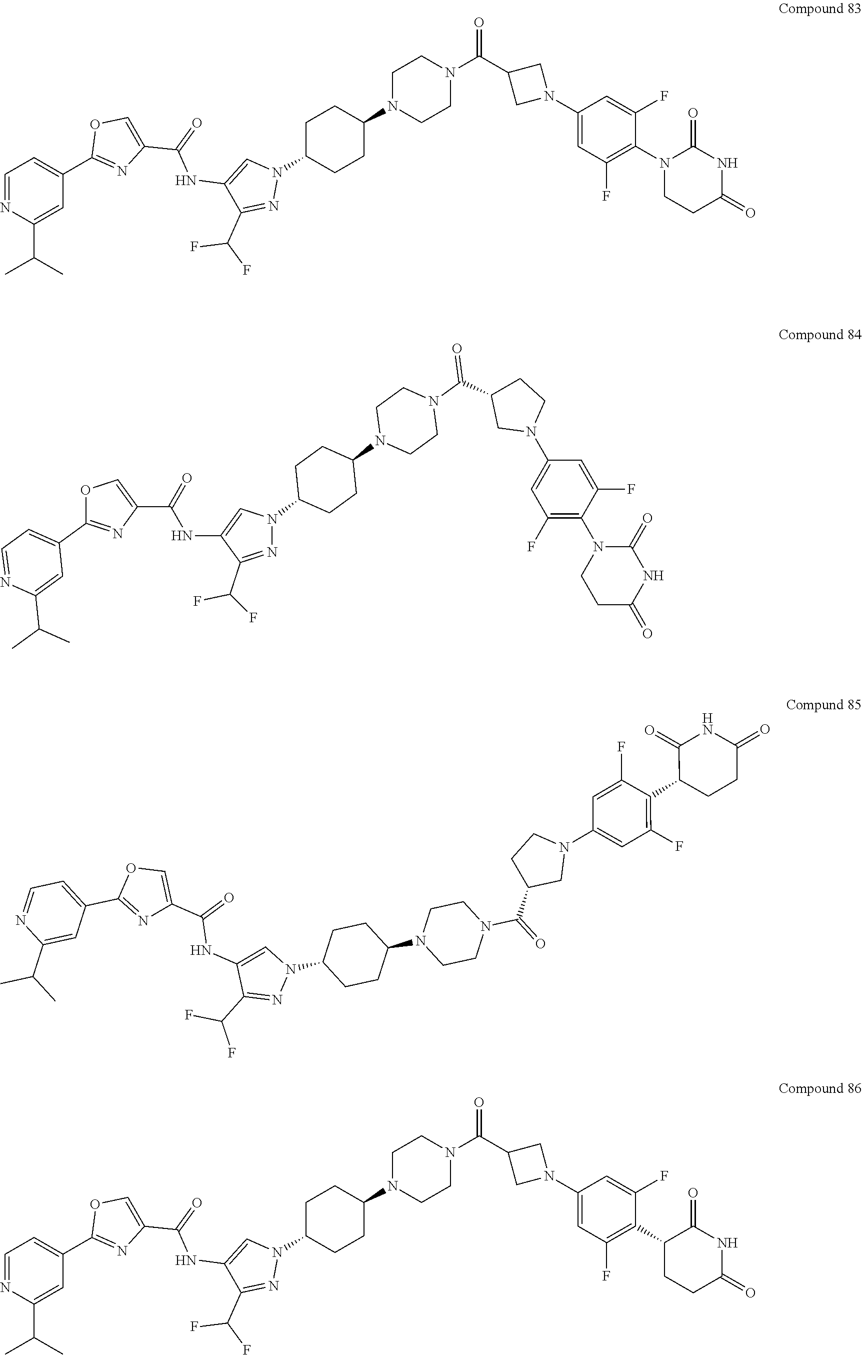 [SMILES: CC(C)c1cc(-c2nc(C(=O)Nc3cn([C@H]4CC[C@H](N5CCN(C(=O)C6CN(c7cc(F)c(N8CCC(=O)NC8=O)c(F)c7)C6)CC5)CC4)nc3C(F)F)co2)ccn1 ; CC(C)c1cc(-c2nc(C(=O)Nc3cn([C@H]4CC[C@H](N5CCN(C(=O)[C@@H]6CCN(c7cc(F)c(N8CCC(=O)NC8=O)c(F)c7)C6)CC5)CC4)nc3C(F)F)co2)ccn1 ; CC(C)c1cc(-c2nc(C(=O)Nc3cn([C@H]4CC[C@H](N5CCN(C(=O)[C@@H]6CCN(c7cc(F)c([C@H]8CCC(=O)NC8=O)c(F)c7)C6)CC5)CC4)nc3C(F)F)co2)ccn1 ; CC(C)c1cc(-c2nc(C(=O)Nc3cn([C@H]4CC[C@H](N5CCN(C(=O)C6CN(c7cc(F)c([C@H]8CCC(=O)NC8=O)c(F)c7)C6)CC5)CC4)nc3C(F)F)co2)ccn1]

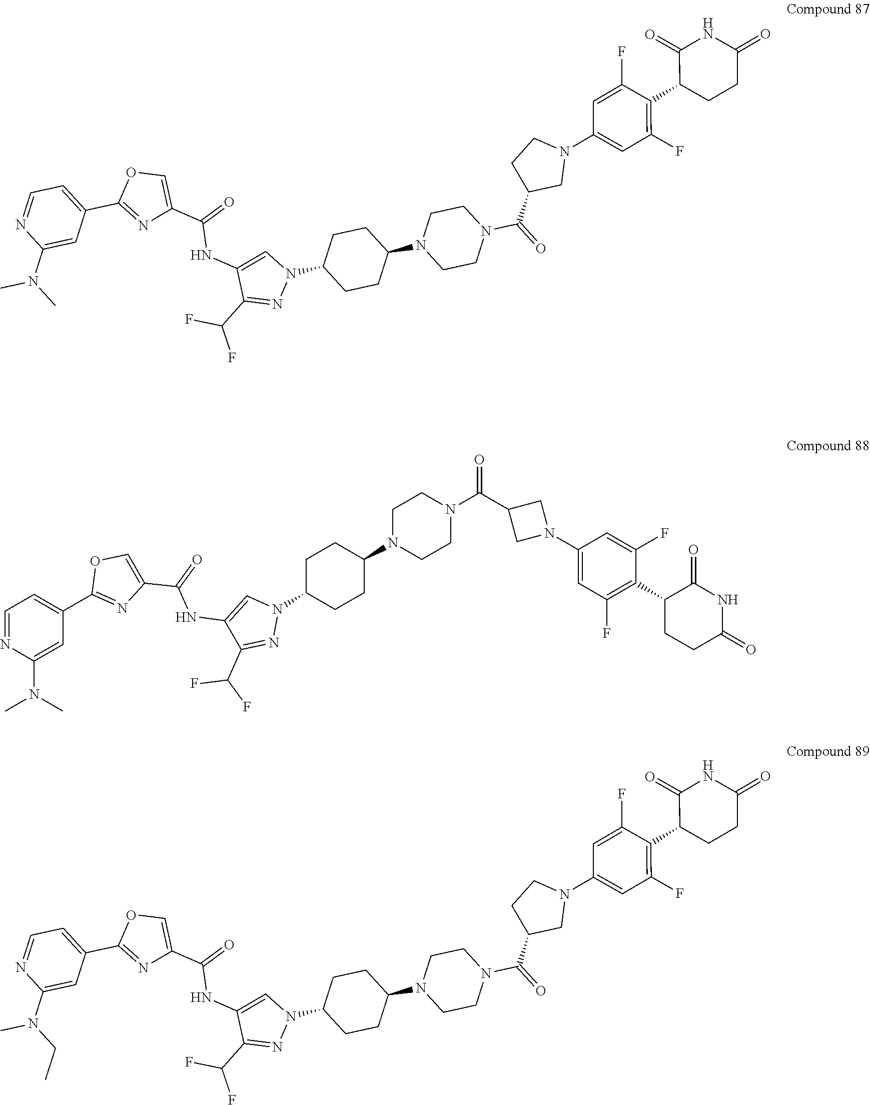 [SMILES: CN(C)c1cc(-c2nc(C(=O)Nc3cn([C@H]4CC[C@H](N5CCN(C(=O)[C@@H]6CCN(c7cc(F)c([C@H]8CCC(=O)NC8=O)c(F)c7)C6)CC5)CC4)nc3C(F)F)co2)ccn1 ; CN(C)c1cc(-c2nc(C(=O)Nc3cn([C@H]4CC[C@H](N5CCN(C(=O)C6CN(c7cc(F)c([C@H]8CCC(=O)NC8=O)c(F)c7)C6)CC5)CC4)nc3C(F)F)co2)ccn1 ; CCN(C)c1cc(-c2nc(C(=O)Nc3cn([C@H]4CC[C@H](N5CCN(C(=O)[C@@H]6CCN(c7cc(F)c([C@H]8CCC(=O)NC8=O)c(F)c7)C6)CC5)CC4)nc3C(F)F)co2)ccn1]

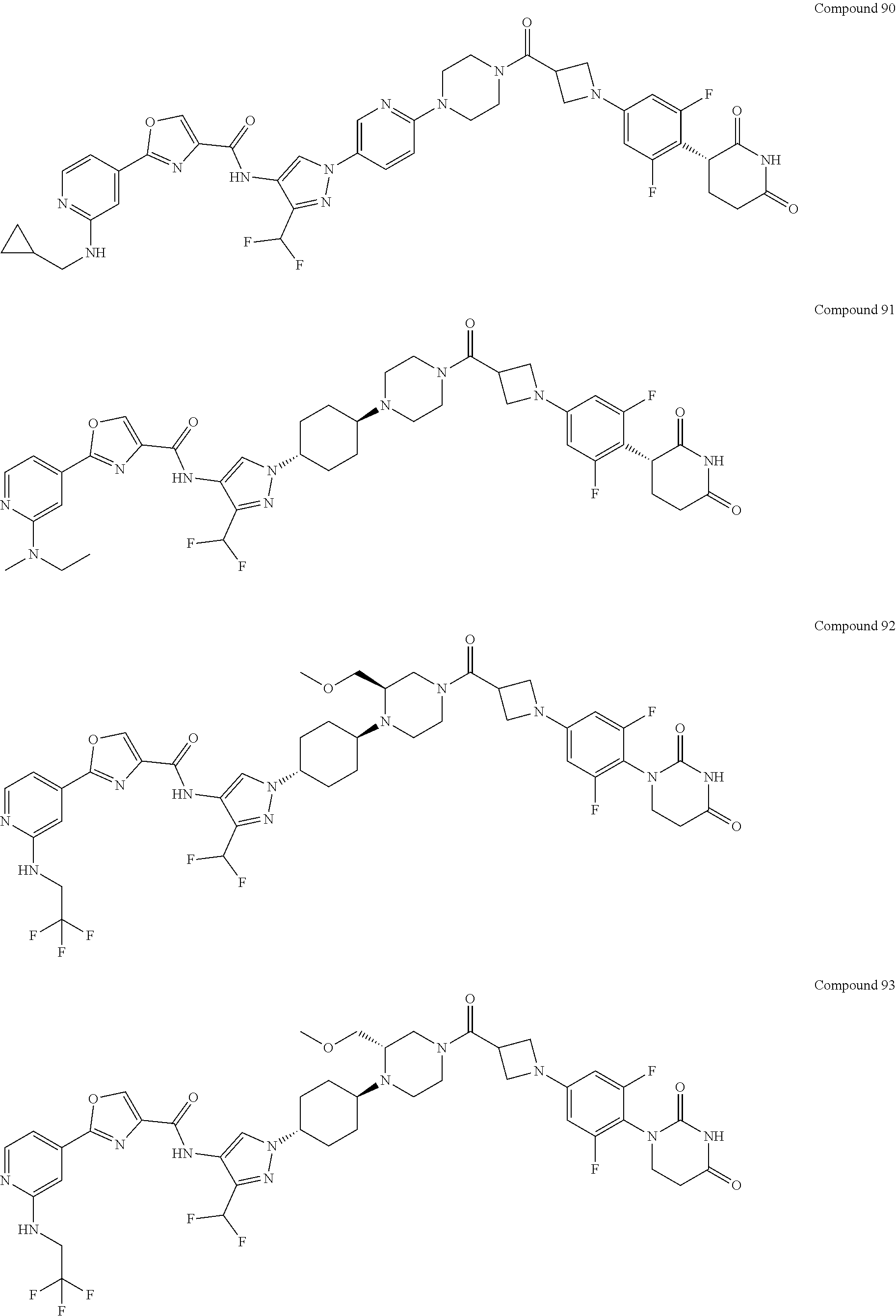

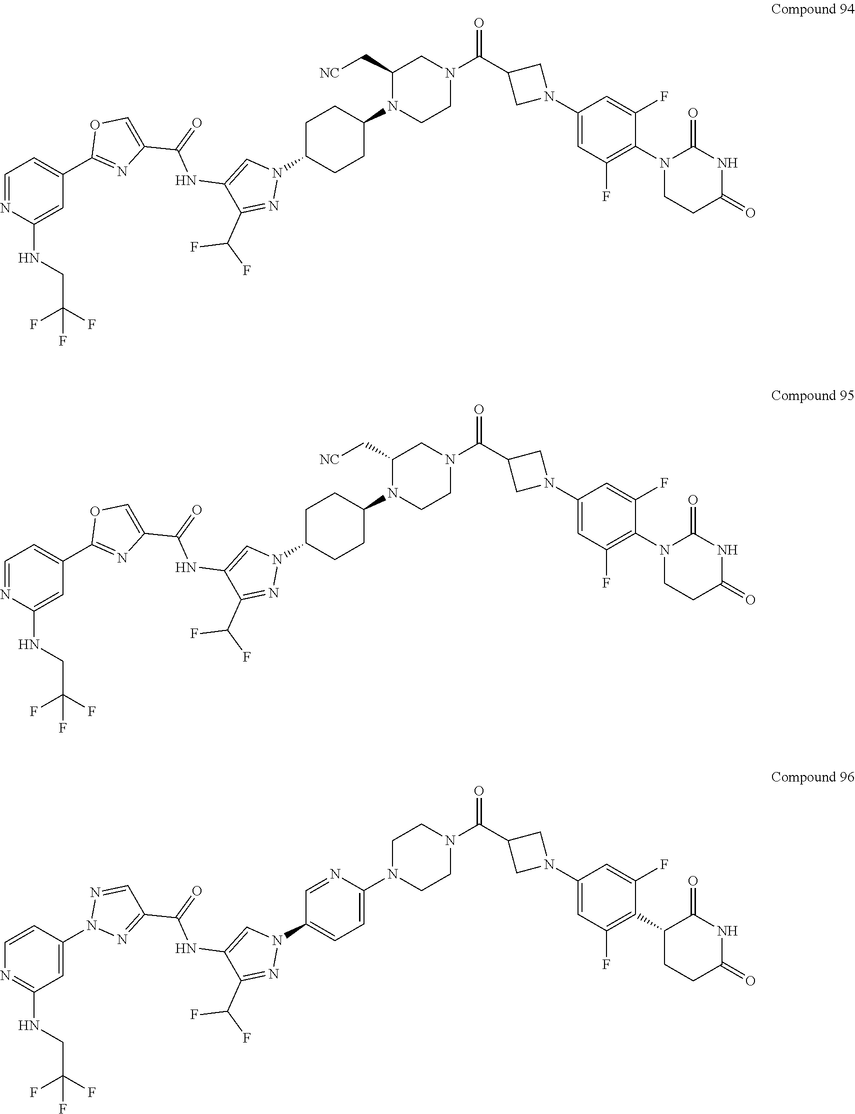

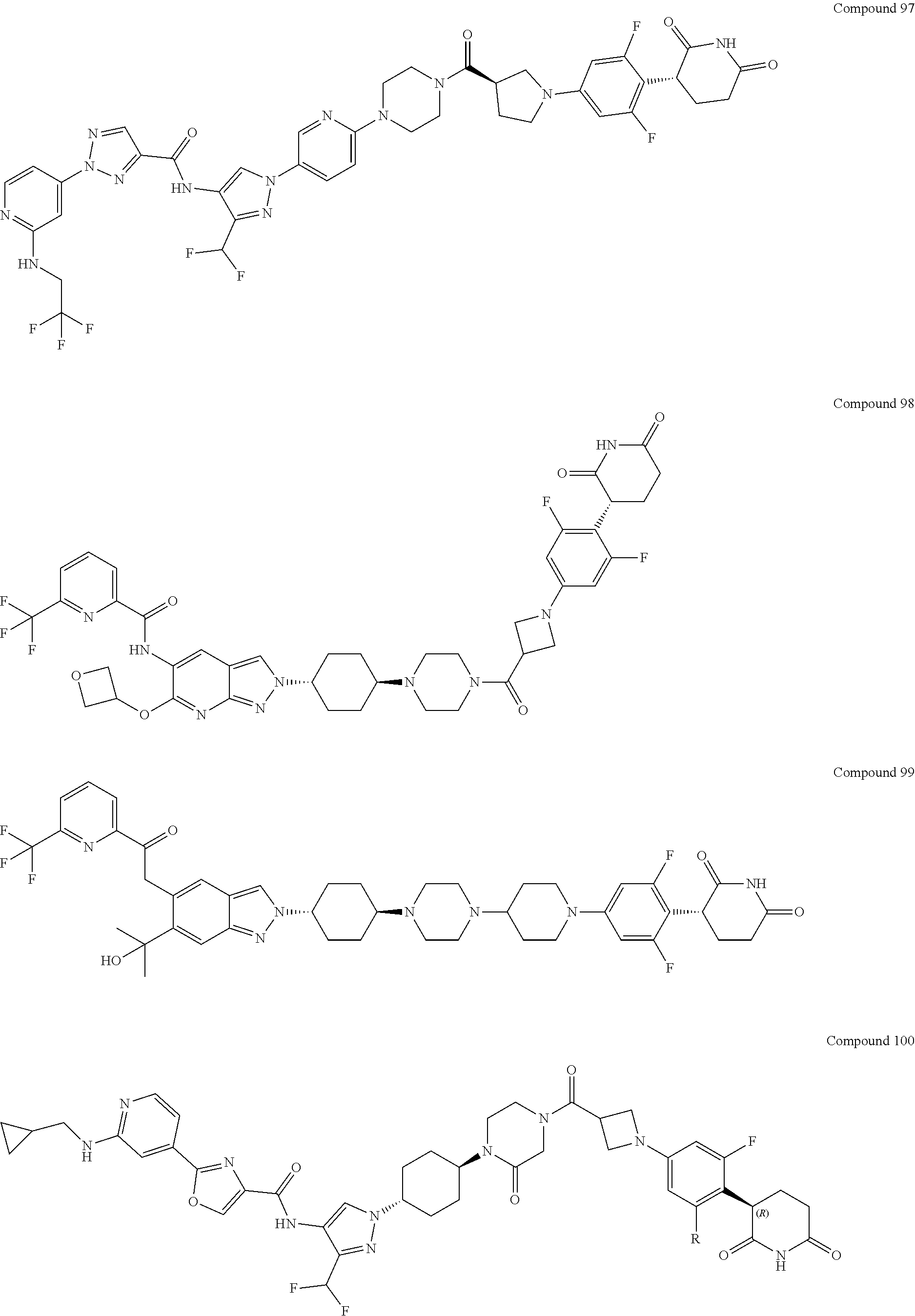

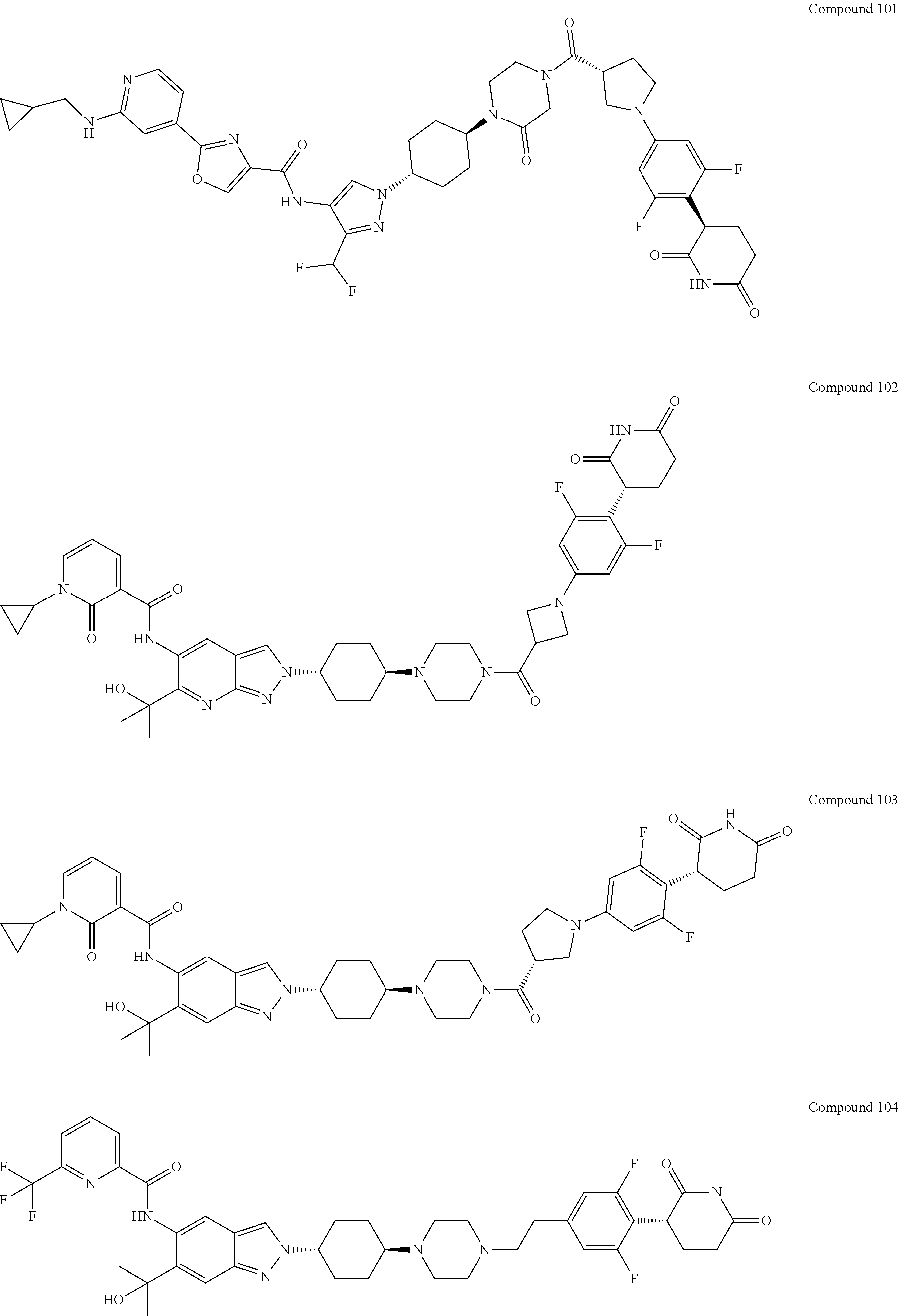

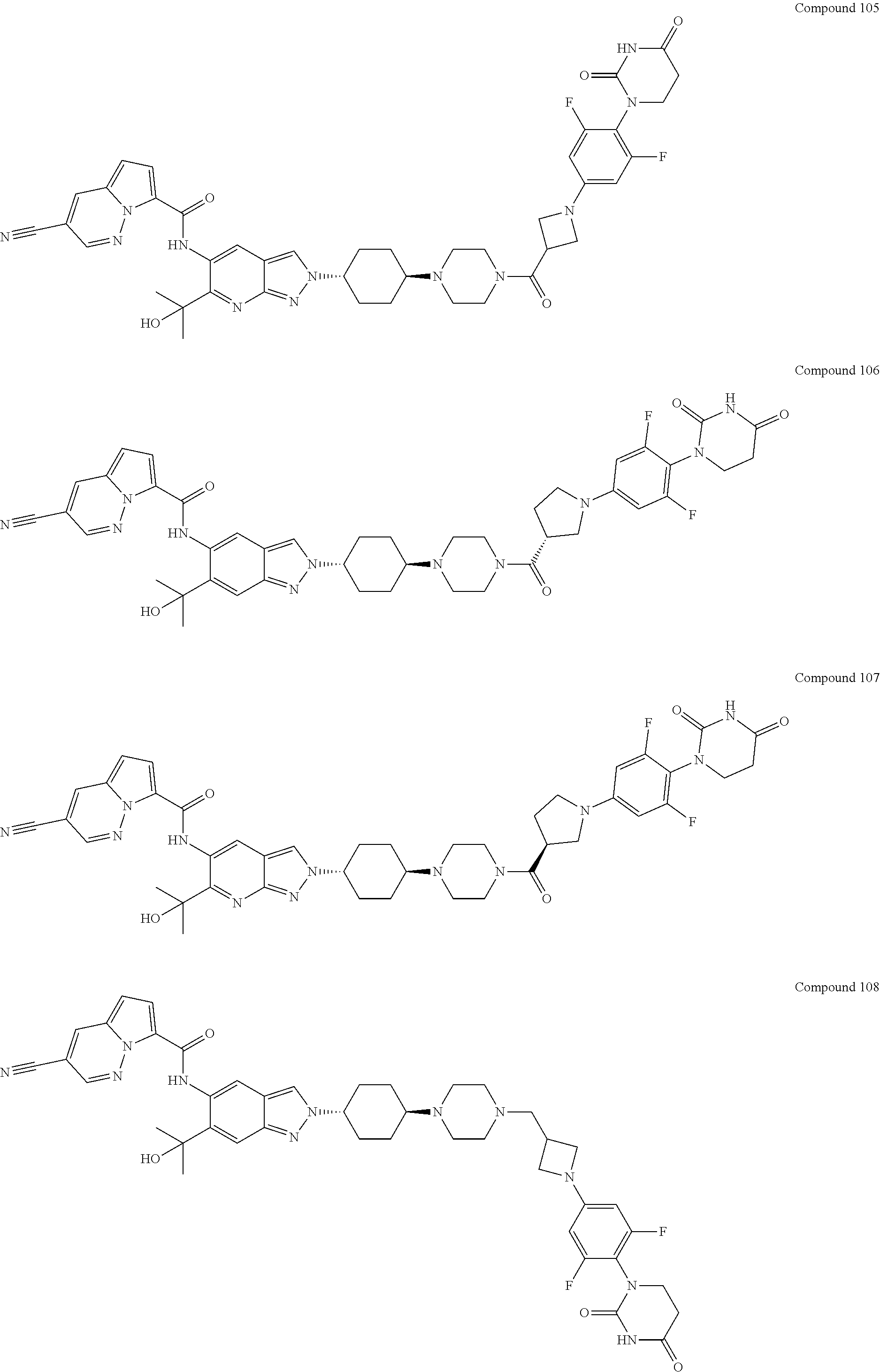 [SMILES: CC(C)(O)c1nc2nn([C@H]3CC[C@H](N4CCN(C(=O)C5CN(c6cc(F)c(N7CCC(=O)NC7=O)c(F)c6)C5)CC4)CC3)cc2cc1NC(=O)c1ccc2cc(C#N)cnn12 ; CC(C)(O)c1cc2nn([C@H]3CC[C@H](N4CCN(C(=O)[C@@H]5CCN(c6cc(F)c(N7CCC(=O)NC7=O)c(F)c6)C5)CC4)CC3)cc2cc1NC(=O)c1ccc2cc(C#N)cnn12 ; CC(C)(O)c1nc2nn([C@H]3CC[C@H](N4CCN(C(=O)[C@H]5CCN(c6cc(F)c(N7CCC(=O)NC7=O)c(F)c6)C5)CC4)CC3)cc2cc1NC(=O)c1ccc2cc(C#N)cnn12 ; CC(C)(O)c1cc2nn([C@H]3CC[C@H](N4CCN(CC5CN(c6cc(F)c(N7CCC(=O)NC7=O)c(F)c6)C5)CC4)CC3)cc2cc1NC(=O)c1ccc2cc(C#N)cnn12]

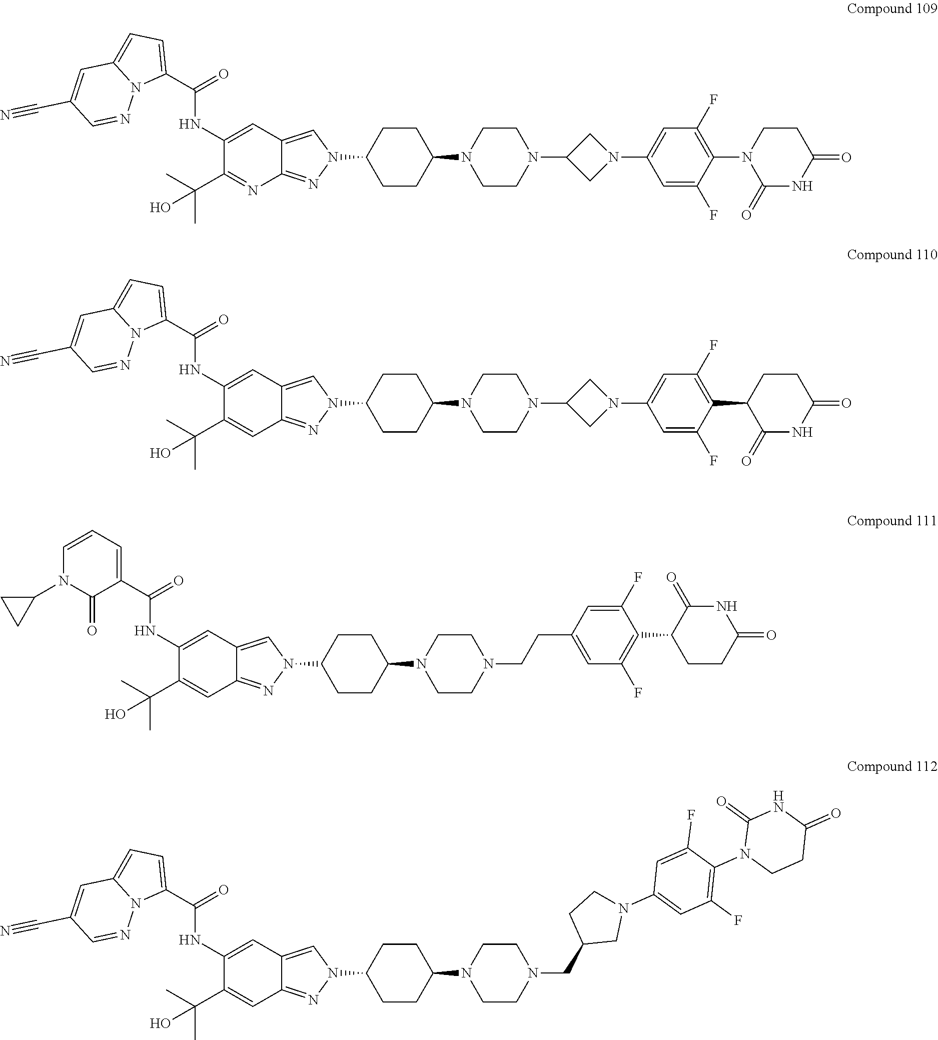

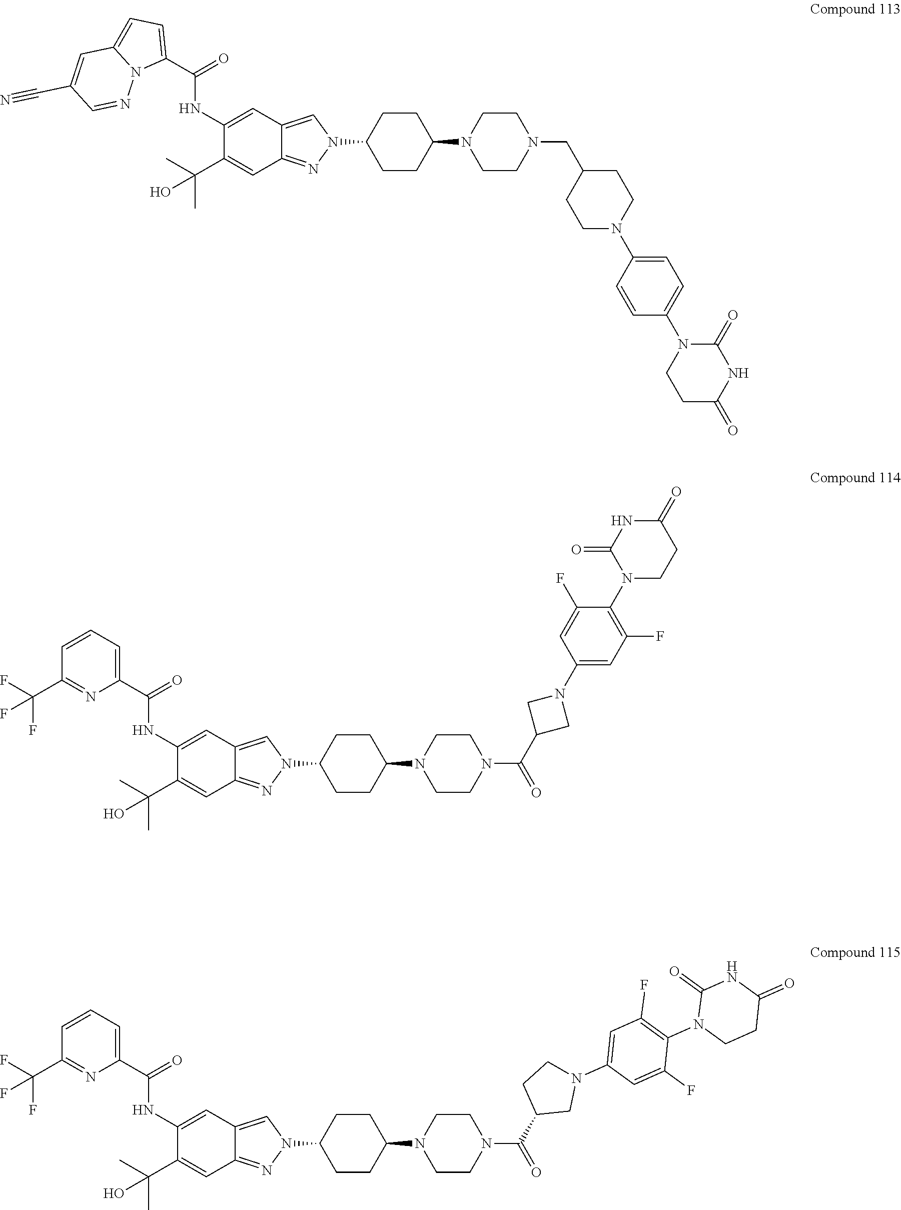 [SMILES: CC(C)(O)c1cc2nn([C@H]3CC[C@H](N4CCN(CC5CCN(c6ccc(N7CCC(=O)NC7=O)cc6)CC5)CC4)CC3)cc2cc1NC(=O)c1ccc2cc(C#N)cnn12 ; CC(C)(O)c1cc2nn([C@H]3CC[C@H](N4CCN(C(=O)C5CN(c6cc(F)c(N7CCC(=O)NC7=O)c(F)c6)C5)CC4)CC3)cc2cc1NC(=O)c1cccc(C(F)(F)F)n1 ; CC(C)(O)c1cc2nn([C@H]3CC[C@H](N4CCN(C(=O)[C@@H]5CCN(c6cc(F)c(N7CCC(=O)NC7=O)c(F)c6)C5)CC4)CC3)cc2cc1NC(=O)c1cccc(C(F)(F)F)n1]

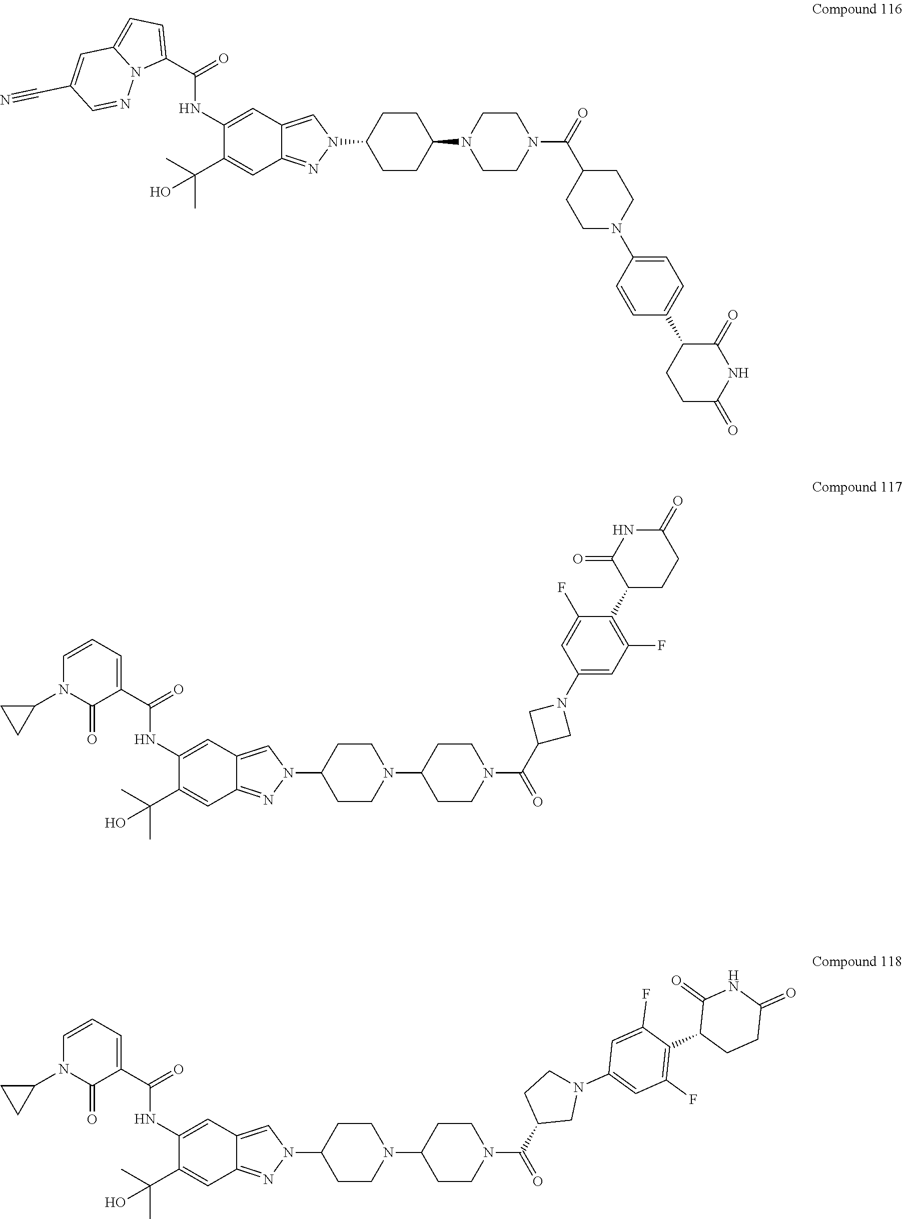 [SMILES: CC(C)(O)c1cc2nn([C@H]3CC[C@H](N4CCN(C(=O)C5CCN(c6ccc([C@H]7CCC(=O)NC7=O)cc6)CC5)CC4)CC3)cc2cc1NC(=O)c1ccc2cc(C#N)cnn12 ; CC(C)(O)c1cc2nn(C3CCN(C4CCN(C(=O)C5CN(c6cc(F)c([C@H]7CCC(=O)NC7=O)c(F)c6)C5)CC4)CC3)cc2cc1NC(=O)c1cccn(C2CC2)c1=O ; CC(C)(O)c1cc2nn(C3CCN(C4CCN(C(=O)[C@@H]5CCN(c6cc(F)c([C@H]7CCC(=O)NC7=O)c(F)c6)C5)CC4)CC3)cc2cc1NC(=O)c1cccn(C2CC2)c1=O]

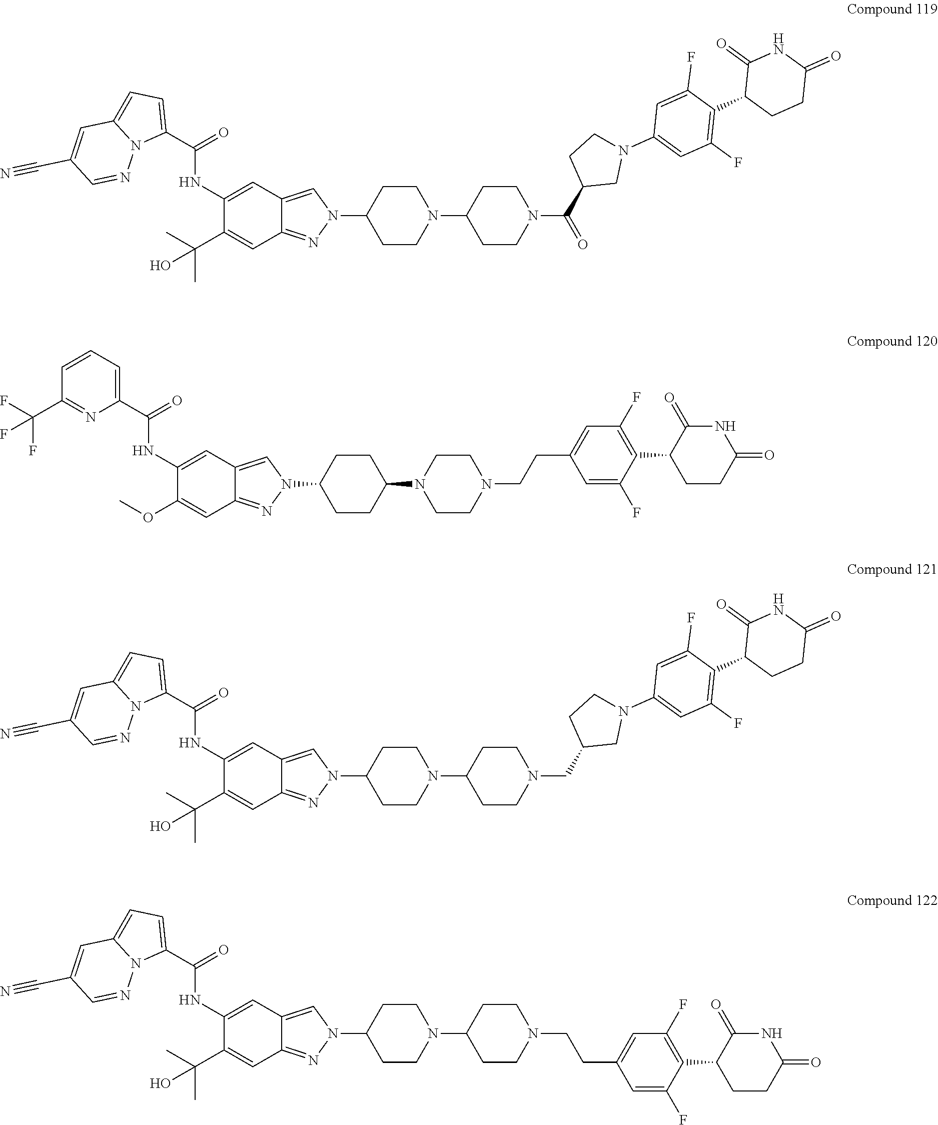 [SMILES: CC(C)(O)c1cc2nn(C3CCN(C4CCN(C(=O)[C@H]5CCN(c6cc(F)c([C@H]7CCC(=O)NC7=O)c(F)c6)C5)CC4)CC3)cc2cc1NC(=O)c1ccc2cc(C#N)cnn12 ; COc1cc2nn([C@H]3CC[C@H](N4CCN(CCc5cc(F)c([C@H]6CCC(=O)NC6=O)c(F)c5)CC4)CC3)cc2cc1NC(=O)c1cccc(C(F)(F)F)n1 ; CC(C)(O)c1cc2nn(C3CCN(C4CCN(C[C@@H]5CCN(c6cc(F)c([C@H]7CCC(=O)NC7=O)c(F)c6)C5)CC4)CC3)cc2cc1NC(=O)c1ccc2cc(C#N)cnn12 ; CC(C)(O)c1cc2nn(C3CCN(C4CCN(CCc5cc(F)c([C@H]6CCC(=O)NC6=O)c(F)c5)CC4)CC3)cc2cc1NC(=O)c1ccc2cc(C#N)cnn12]

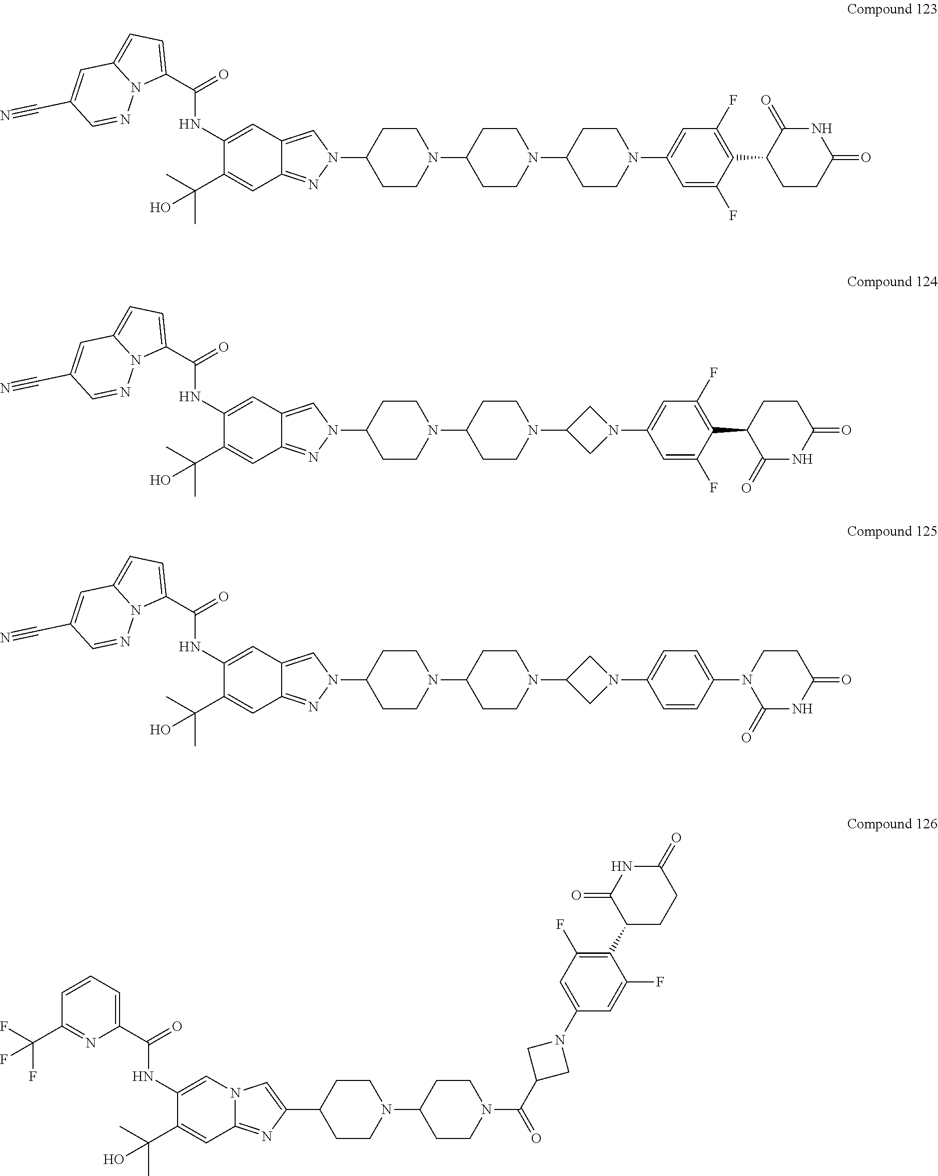 [SMILES: CC(C)(O)c1cc2nn(C3CCN(C4CCN(C5CCN(c6cc(F)c([C@H]7CCC(=O)NC7=O)c(F)c6)CC5)CC4)CC3)cc2cc1NC(=O)c1ccc2cc(C#N)cnn12 ; CC(C)(O)c1cc2nn(C3CCN(C4CCN(C5CN(c6cc(F)c([C@H]7CCC(=O)NC7=O)c(F)c6)C5)CC4)CC3)cc2cc1NC(=O)c1ccc2cc(C#N)cnn12 ; CC(C)(O)c1cc2nn(C3CCN(C4CCN(C5CN(c6ccc(N7CCC(=O)NC7=O)cc6)C5)CC4)CC3)cc2cc1NC(=O)c1ccc2cc(C#N)cnn12 ; CC(C)(O)c1cc2nc(C3CCN(C4CCN(C(=O)C5CN(c6cc(F)c([C@H]7CCC(=O)NC7=O)c(F)c6)C5)CC4)CC3)cn2cc1NC(=O)c1cccc(C(F)(F)F)n1]

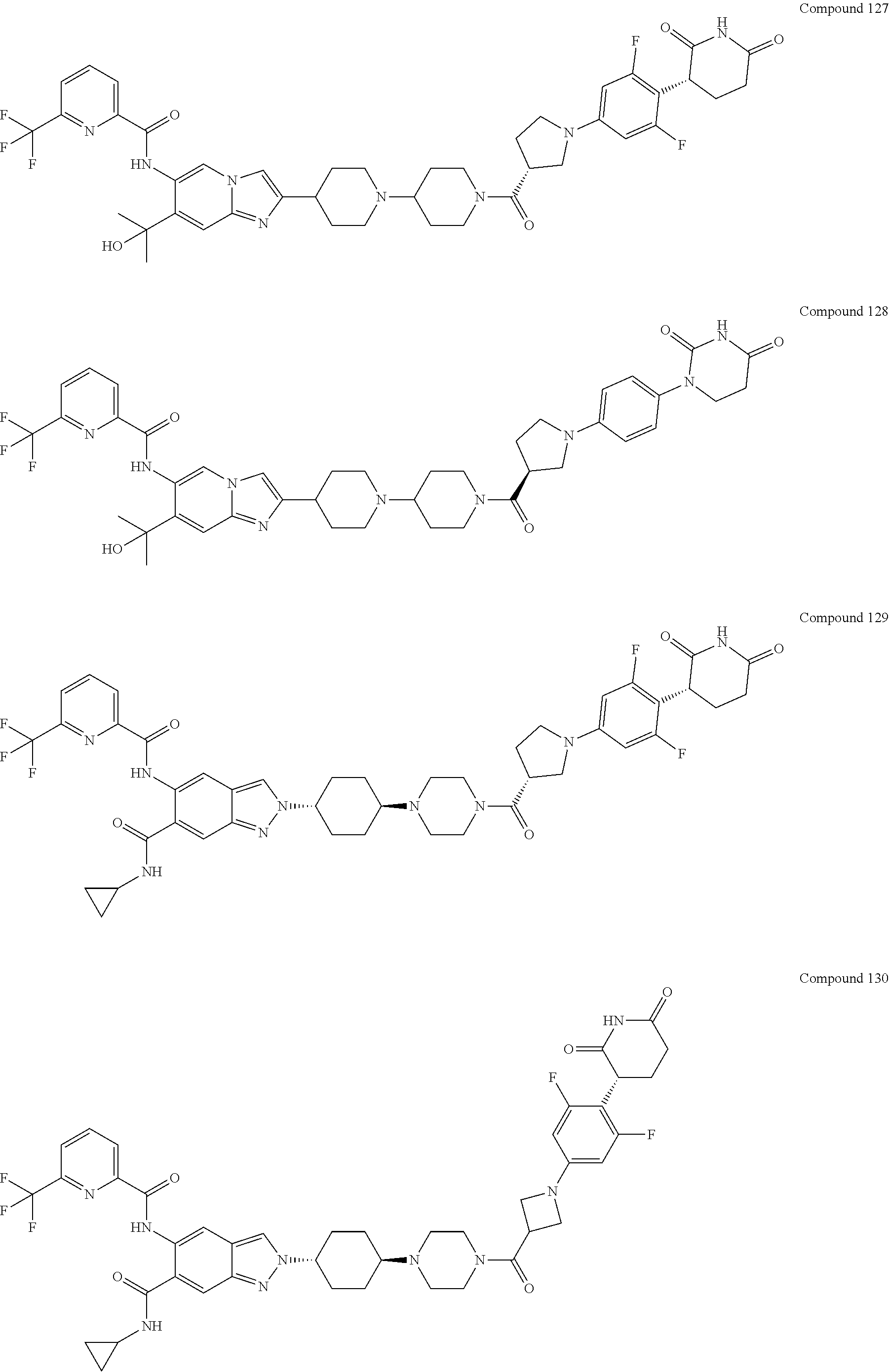 [SMILES: CC(C)(O)c1cc2nc(C3CCN(C4CCN(C(=O)[C@@H]5CCN(c6cc(F)c([C@H]7CCC(=O)NC7=O)c(F)c6)C5)CC4)CC3)cn2cc1NC(=O)c1cccc(C(F)(F)F)n1 ; CC(C)(O)c1cc2nc(C3CCN(C4CCN(C(=O)[C@H]5CCN(c6ccc(N7CCC(=O)NC7=O)cc6)C5)CC4)CC3)cn2cc1NC(=O)c1cccc(C(F)(F)F)n1 ; O=C1CC[C@H](c2c(F)cc(N3CC[C@@H](C(=O)N4CCN([C@H]5CC[C@H](n6cc7cc(NC(=O)c8cccc(C(F)(F)F)n8)c(C(=O)NC8CC8)cc7n6)CC5)CC4)C3)cc2F)C(=O)N1 ; O=C1CC[C@H](c2c(F)cc(N3CC(C(=O)N4CCN([C@H]5CC[C@H](n6cc7cc(NC(=O)c8cccc(C(F)(F)F)n8)c(C(=O)NC8CC8)cc7n6)CC5)CC4)C3)cc2F)C(=O)N1]

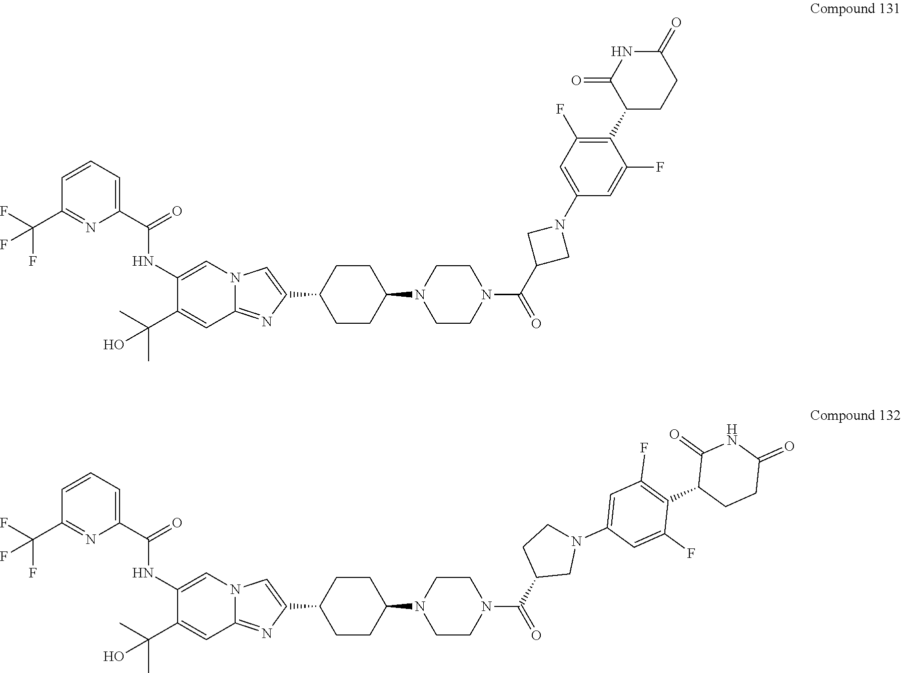 [SMILES: CC(C)(O)c1cc2nc([C@H]3CC[C@H](N4CCN(C(=O)C5CN(c6cc(F)c([C@H]7CCC(=O)NC7=O)c(F)c6)C5)CC4)CC3)cn2cc1NC(=O)c1cccc(C(F)(F)F)n1 ; CC(C)(O)c1cc2nc([C@H]3CC[C@H](N4CCN(C(=O)[C@@H]5CCN(c6cc(F)c([C@H]7CCC(=O)NC7=O)c(F)c6)C5)CC4)CC3)cn2cc1NC(=O)c1cccc(C(F)(F)F)n1]

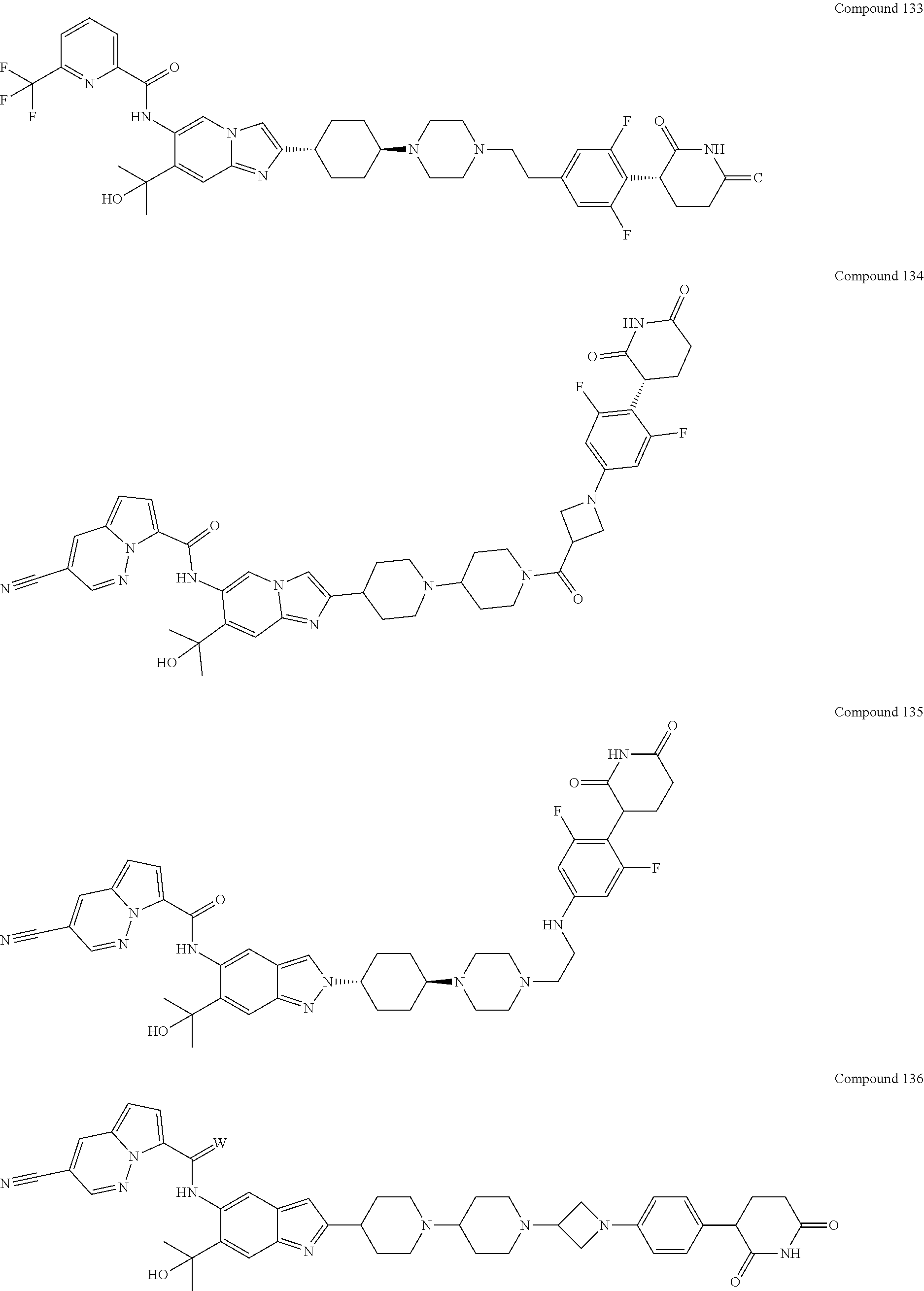

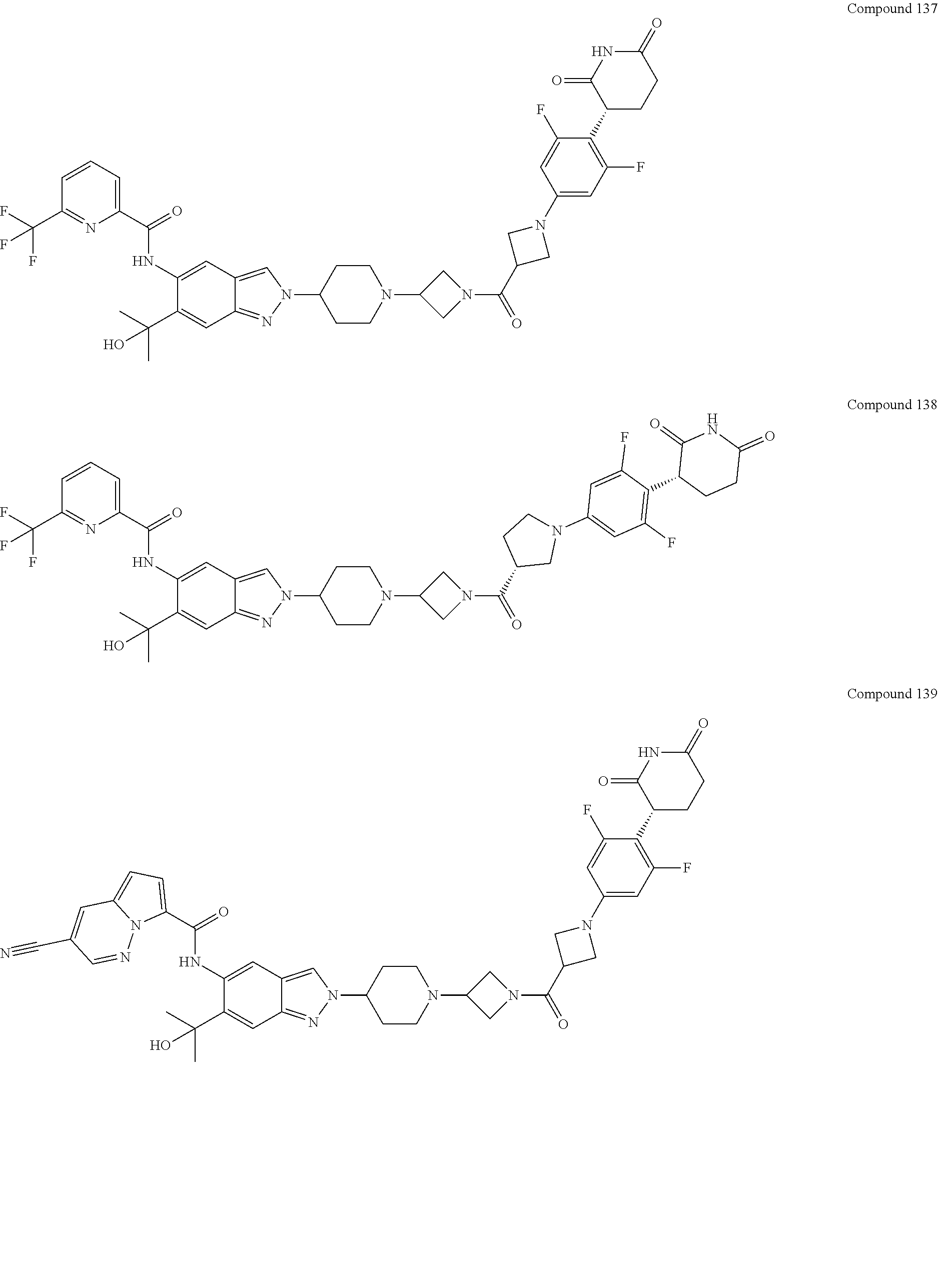 [SMILES: CC(C)(O)c1cc2nn(C3CCN(C4CN(C(=O)C5CN(c6cc(F)c([C@H]7CCC(=O)NC7=O)c(F)c6)C5)C4)CC3)cc2cc1NC(=O)c1cccc(C(F)(F)F)n1 ; CC(C)(O)c1cc2nn(C3CCN(C4CN(C(=O)[C@@H]5CCN(c6cc(F)c([C@H]7CCC(=O)NC7=O)c(F)c6)C5)C4)CC3)cc2cc1NC(=O)c1cccc(C(F)(F)F)n1 ; CC(C)(O)c1cc2nn(C3CCN(C4CN(C(=O)C5CN(c6cc(F)c([C@H]7CCC(=O)NC7=O)c(F)c6)C5)C4)CC3)cc2cc1NC(=O)c1ccc2cc(C#N)cnn12]

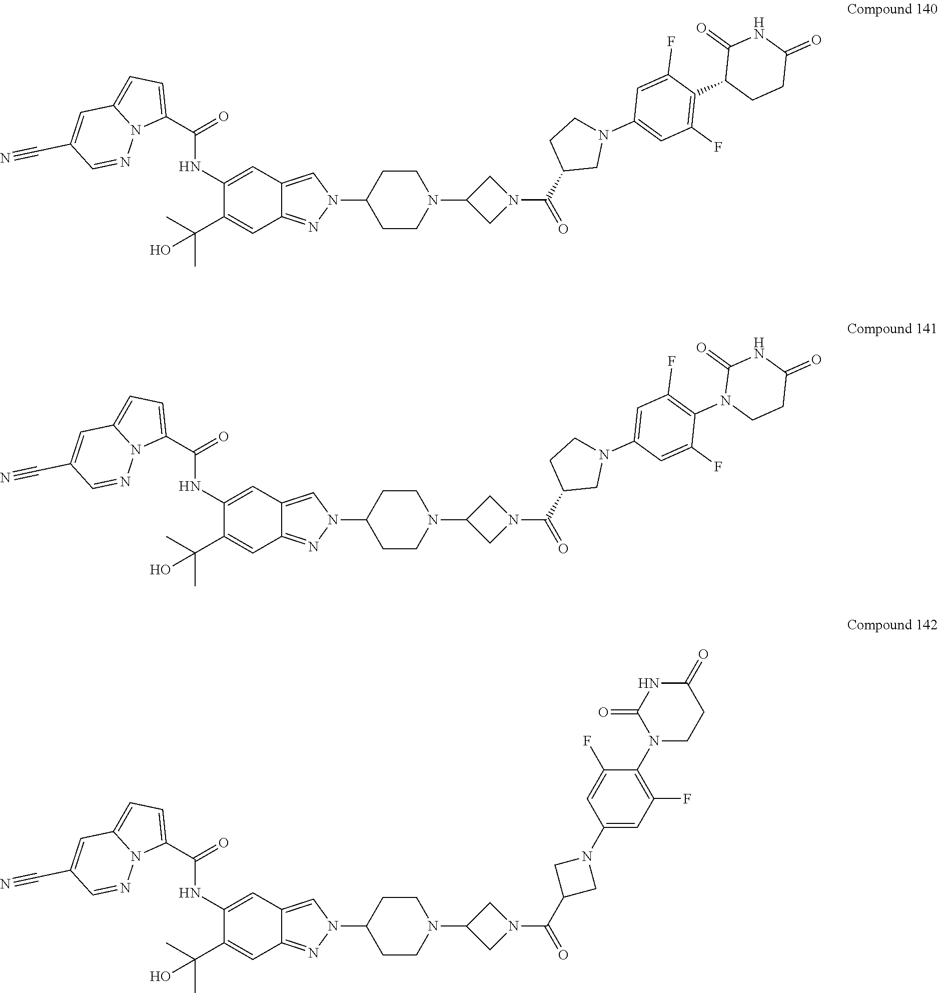 [SMILES: CC(C)(O)c1cc2nn(C3CCN(C4CN(C(=O)[C@@H]5CCN(c6cc(F)c([C@H]7CCC(=O)NC7=O)c(F)c6)C5)C4)CC3)cc2cc1NC(=O)c1ccc2cc(C#N)cnn12 ; CC(C)(O)c1cc2nn(C3CCN(C4CN(C(=O)[C@@H]5CCN(c6cc(F)c(N7CCC(=O)NC7=O)c(F)c6)C5)C4)CC3)cc2cc1NC(=O)c1ccc2cc(C#N)cnn12 ; CC(C)(O)c1cc2nn(C3CCN(C4CN(C(=O)C5CN(c6cc(F)c(N7CCC(=O)NC7=O)c(F)c6)C5)C4)CC3)cc2cc1NC(=O)c1ccc2cc(C#N)cnn12]

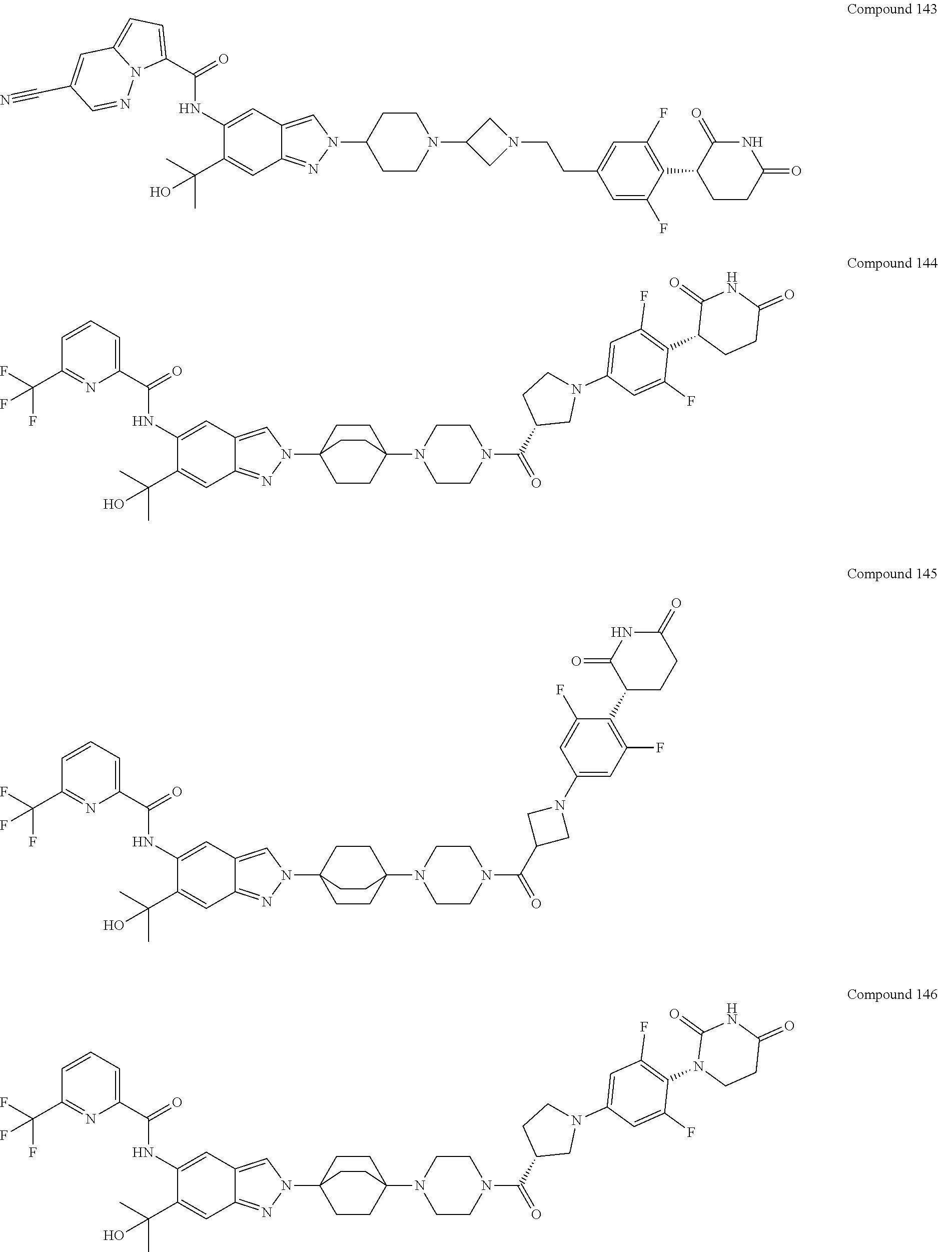

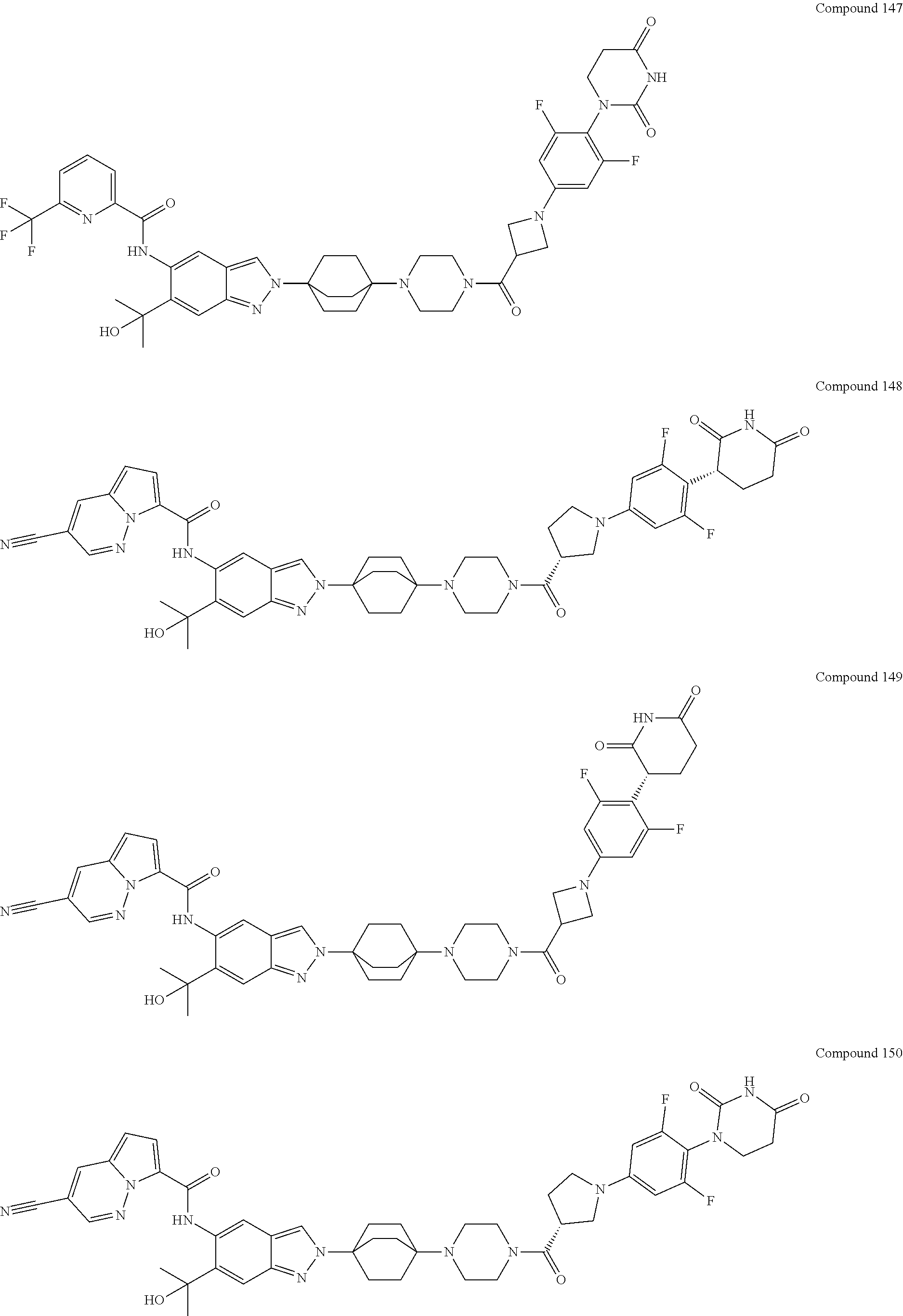 [SMILES: CC(C)(O)c1cc2nn(C34CCC(N5CCN(C(=O)C6CN(c7cc(F)c(N8CCC(=O)NC8=O)c(F)c7)C6)CC5)(CC3)CC4)cc2cc1NC(=O)c1cccc(C(F)(F)F)n1 ; CC(C)(O)c1cc2nn(C34CCC(N5CCN(C(=O)[C@@H]6CCN(c7cc(F)c([C@H]8CCC(=O)NC8=O)c(F)c7)C6)CC5)(CC3)CC4)cc2cc1NC(=O)c1ccc2cc(C#N)cnn12 ; CC(C)(O)c1cc2nn(C34CCC(N5CCN(C(=O)C6CN(c7cc(F)c([C@H]8CCC(=O)NC8=O)c(F)c7)C6)CC5)(CC3)CC4)cc2cc1NC(=O)c1ccc2cc(C#N)cnn12 ; CC(C)(O)c1cc2nn(C34CCC(N5CCN(C(=O)[C@@H]6CCN(c7cc(F)c(N8CCC(=O)NC8=O)c(F)c7)C6)CC5)(CC3)CC4)cc2cc1NC(=O)c1ccc2cc(C#N)cnn12]

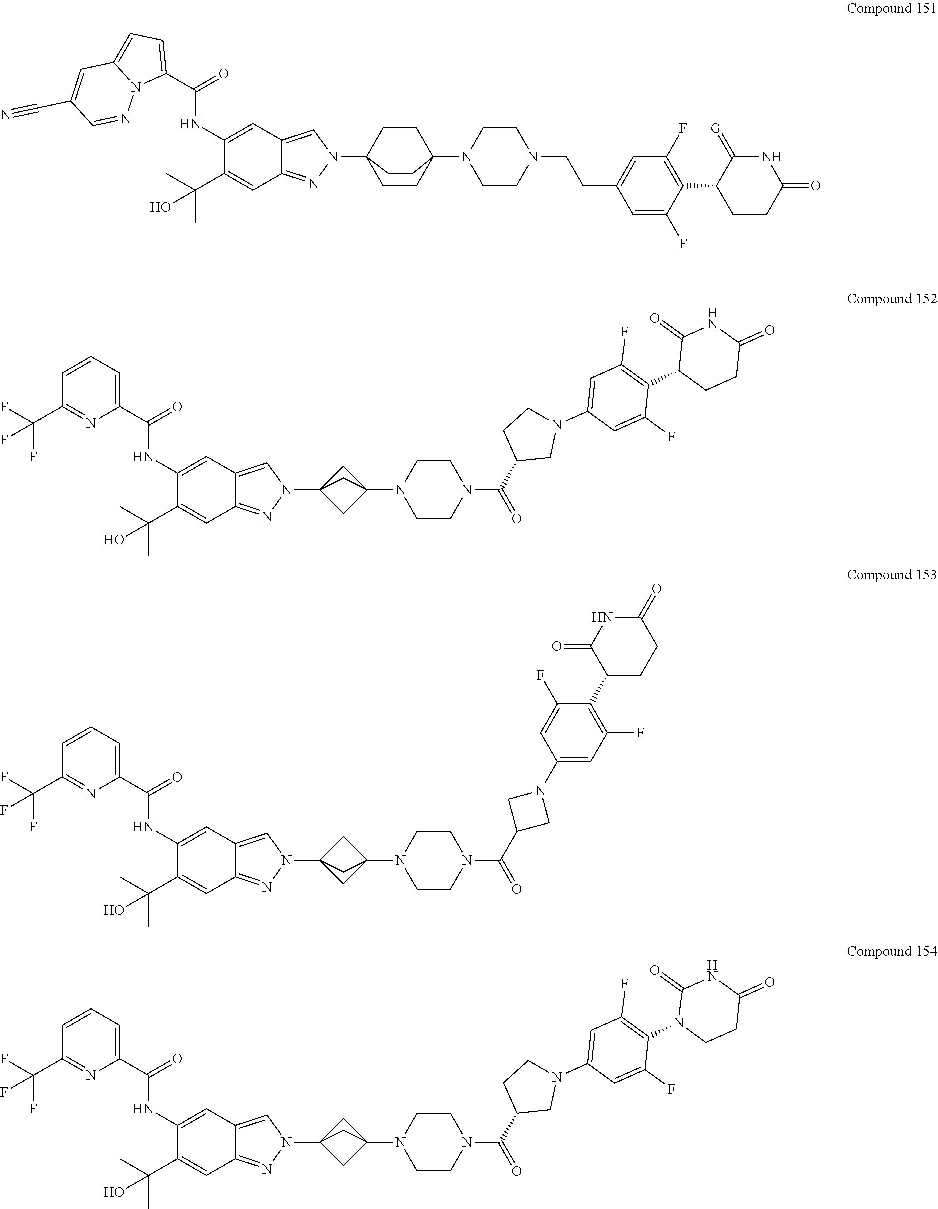

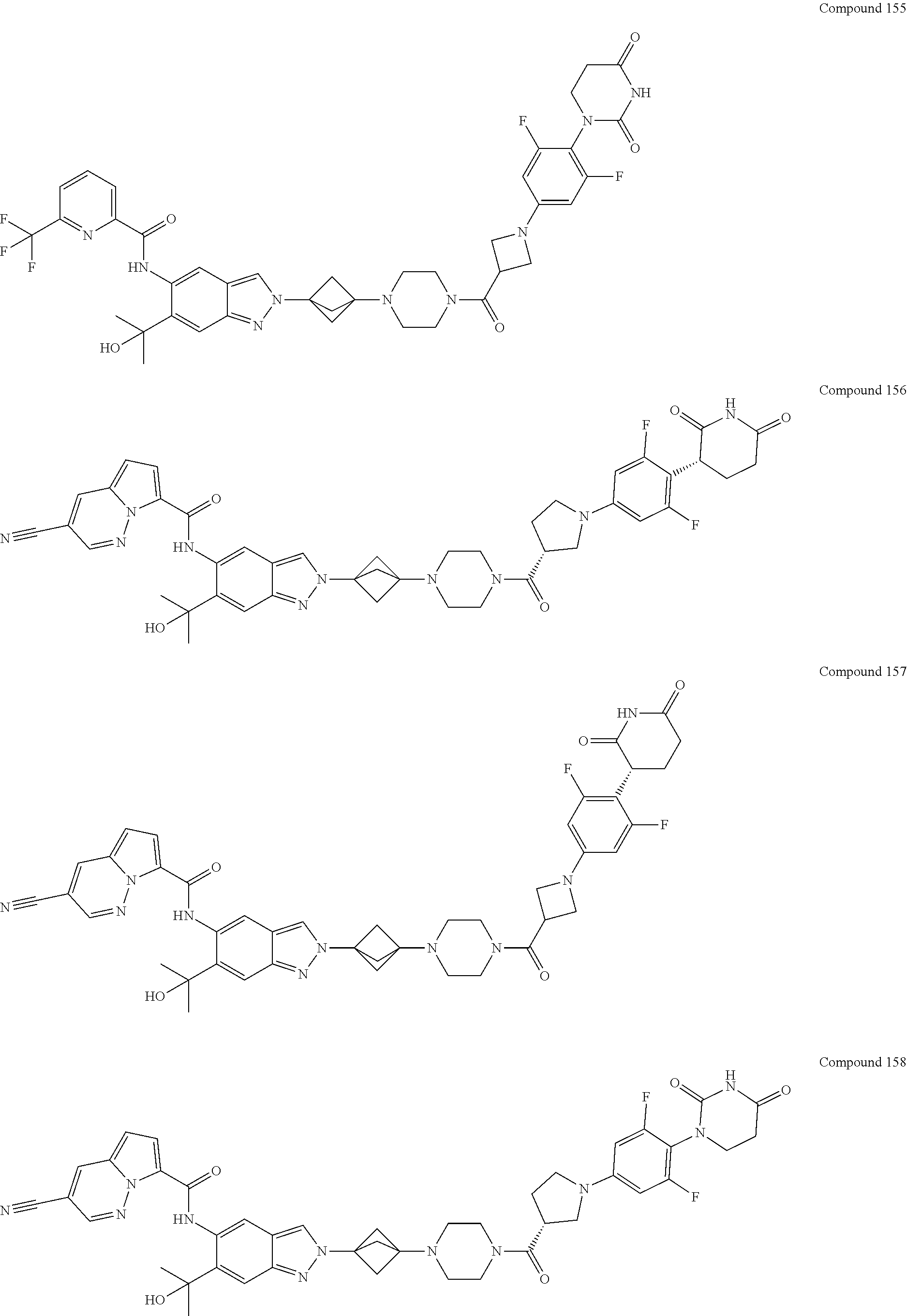 [SMILES: CC(C)(O)c1cc2nn(C34CC(N5CCN(C(=O)C6CN(c7cc(F)c(N8CCC(=O)NC8=O)c(F)c7)C6)CC5)(C3)C4)cc2cc1NC(=O)c1cccc(C(F)(F)F)n1 ; CC(C)(O)c1cc2nn(C34CC(N5CCN(C(=O)[C@@H]6CCN(c7cc(F)c([C@H]8CCC(=O)NC8=O)c(F)c7)C6)CC5)(C3)C4)cc2cc1NC(=O)c1ccc2cc(C#N)cnn12 ; CC(C)(O)c1cc2nn(C34CC(N5CCN(C(=O)C6CN(c7cc(F)c([C@H]8CCC(=O)NC8=O)c(F)c7)C6)CC5)(C3)C4)cc2cc1NC(=O)c1ccc2cc(C#N)cnn12 ; CC(C)(O)c1cc2nn(C34CC(N5CCN(C(=O)[C@@H]6CCN(c7cc(F)c(N8CCC(=O)NC8=O)c(F)c7)C6)CC5)(C3)C4)cc2cc1NC(=O)c1ccc2cc(C#N)cnn12]

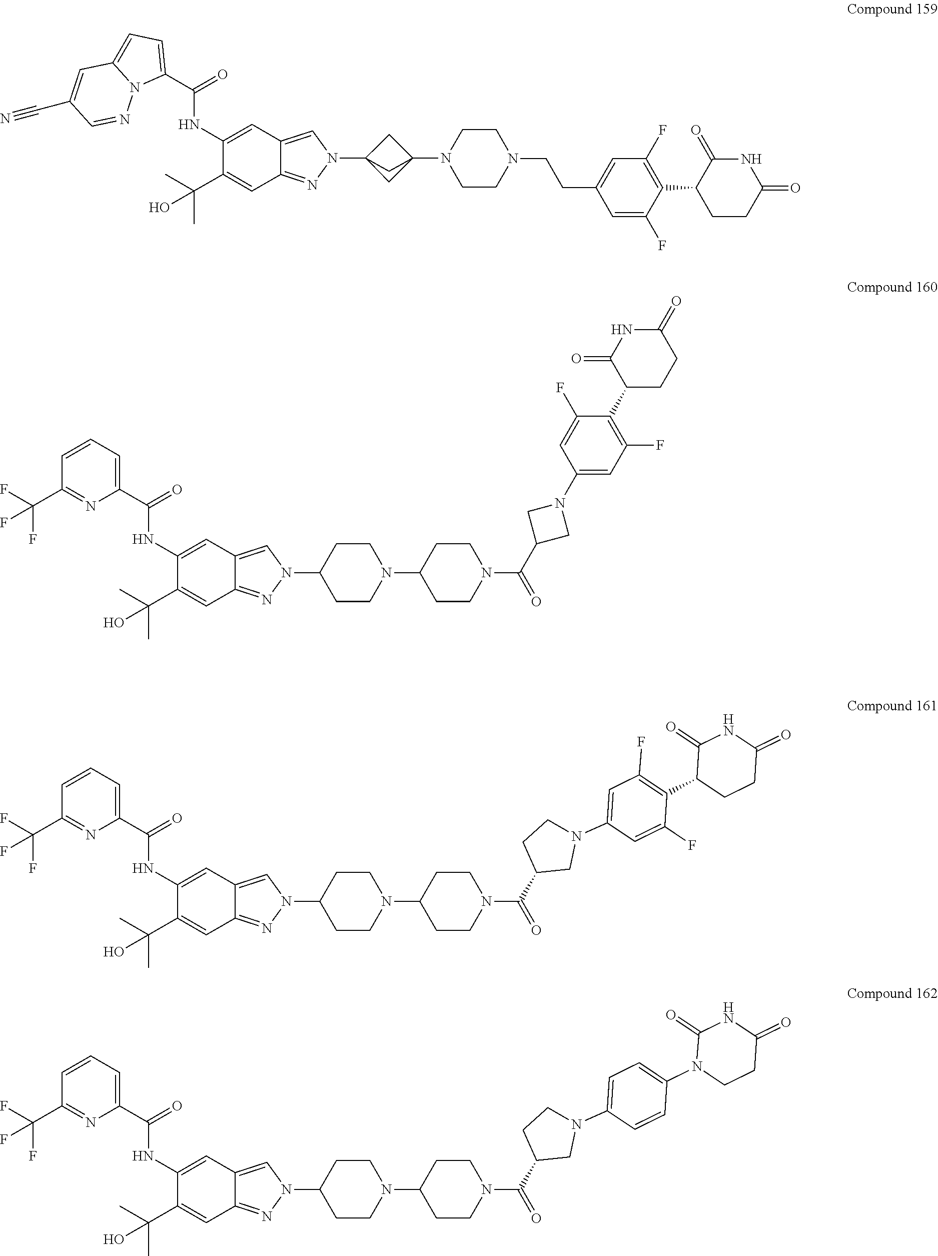 [SMILES: CC(C)(O)c1cc2nn(C34CC(N5CCN(CCc6cc(F)c([C@H]7CCC(=O)NC7=O)c(F)c6)CC5)(C3)C4)cc2cc1NC(=O)c1ccc2cc(C#N)cnn12 ; CC(C)(O)c1cc2nn(C3CCN(C4CCN(C(=O)C5CN(c6cc(F)c([C@H]7CCC(=O)NC7=O)c(F)c6)C5)CC4)CC3)cc2cc1NC(=O)c1cccc(C(F)(F)F)n1 ; CC(C)(O)c1cc2nn(C3CCN(C4CCN(C(=O)[C@@H]5CCN(c6cc(F)c([C@H]7CCC(=O)NC7=O)c(F)c6)C5)CC4)CC3)cc2cc1NC(=O)c1cccc(C(F)(F)F)n1 ; CC(C)(O)c1cc2nn(C3CCN(C4CCN(C(=O)[C@@H]5CCN(c6ccc(N7CCC(=O)NC7=O)cc6)C5)CC4)CC3)cc2cc1NC(=O)c1cccc(C(F)(F)F)n1]

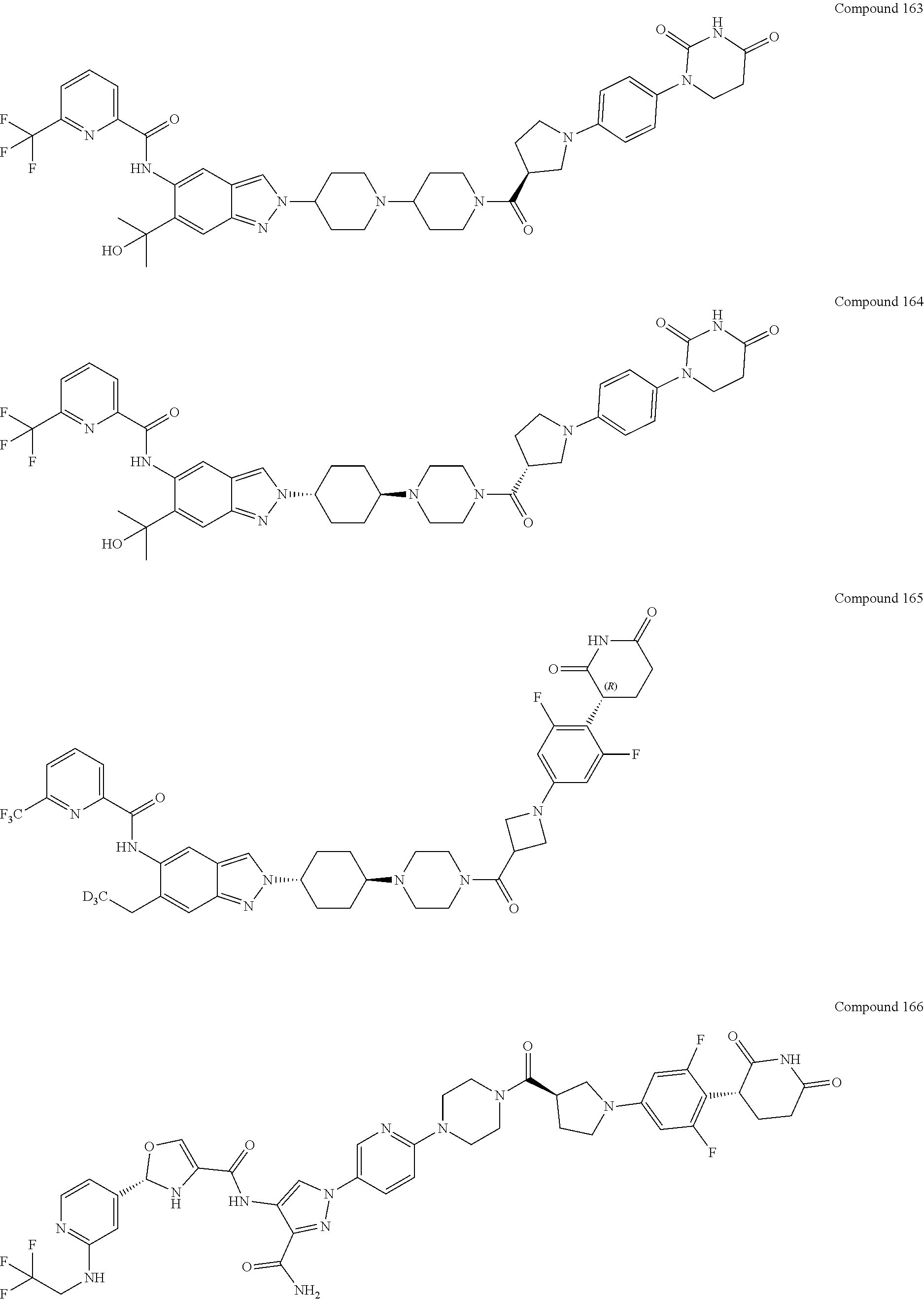

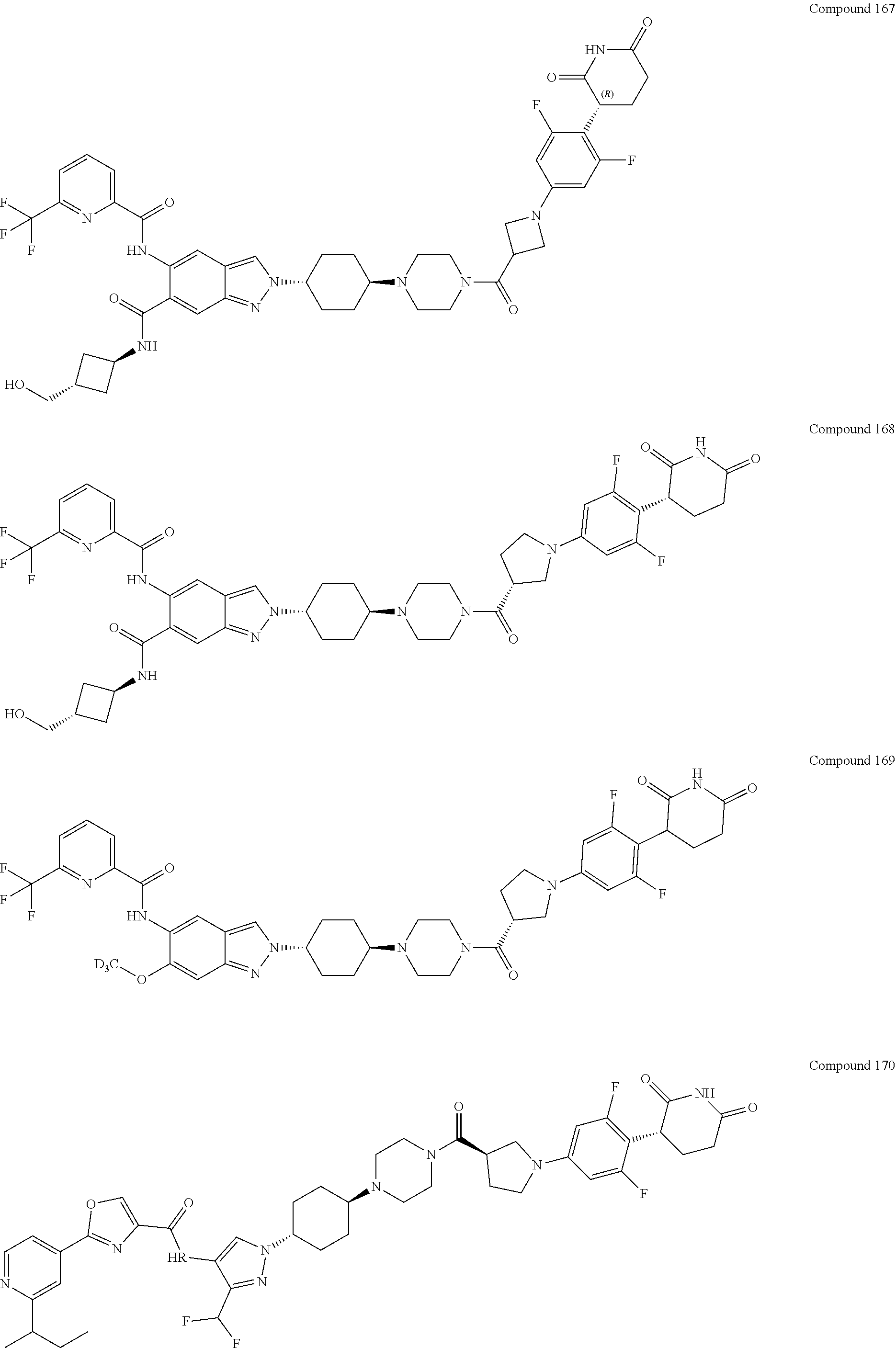

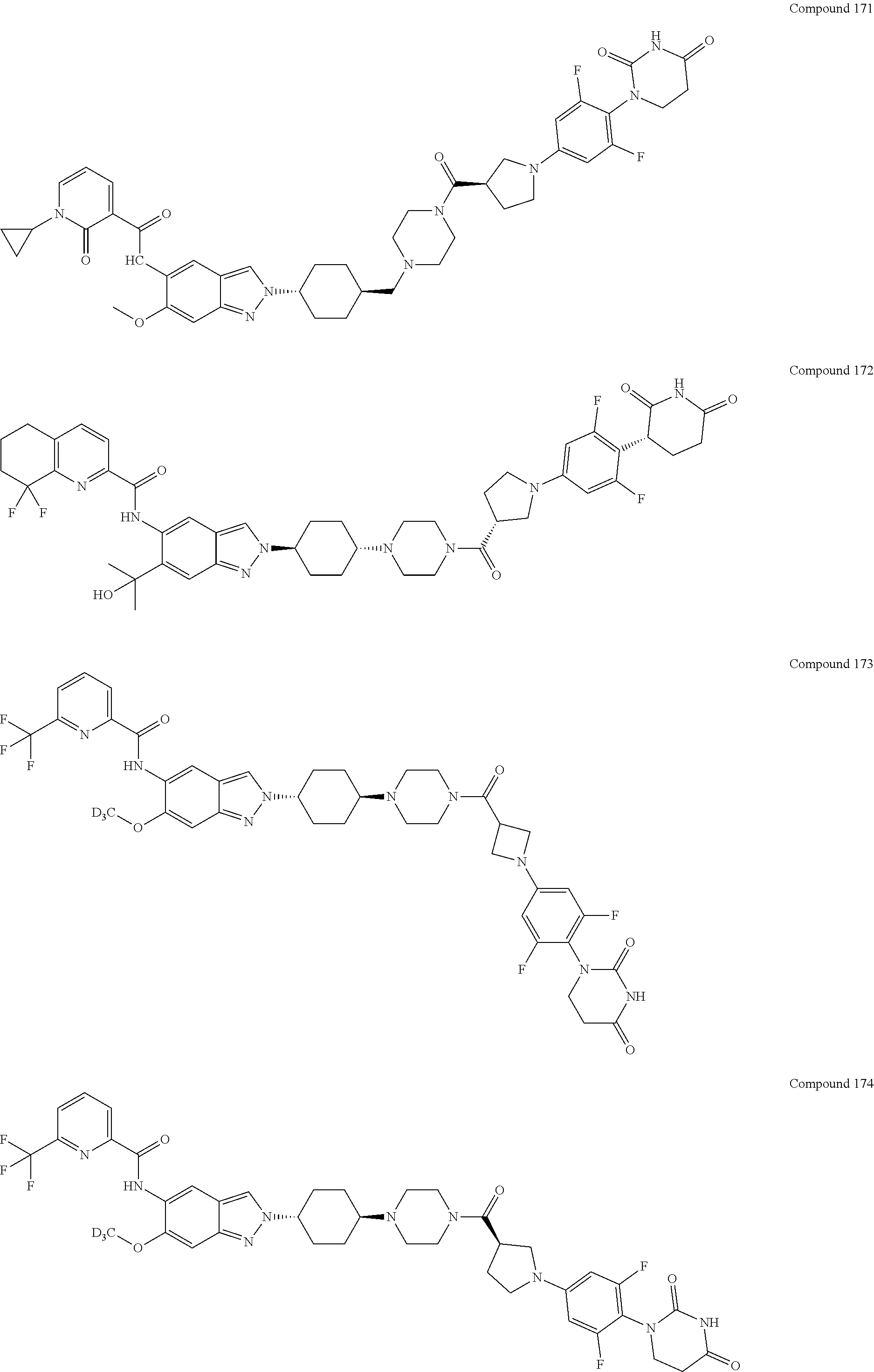

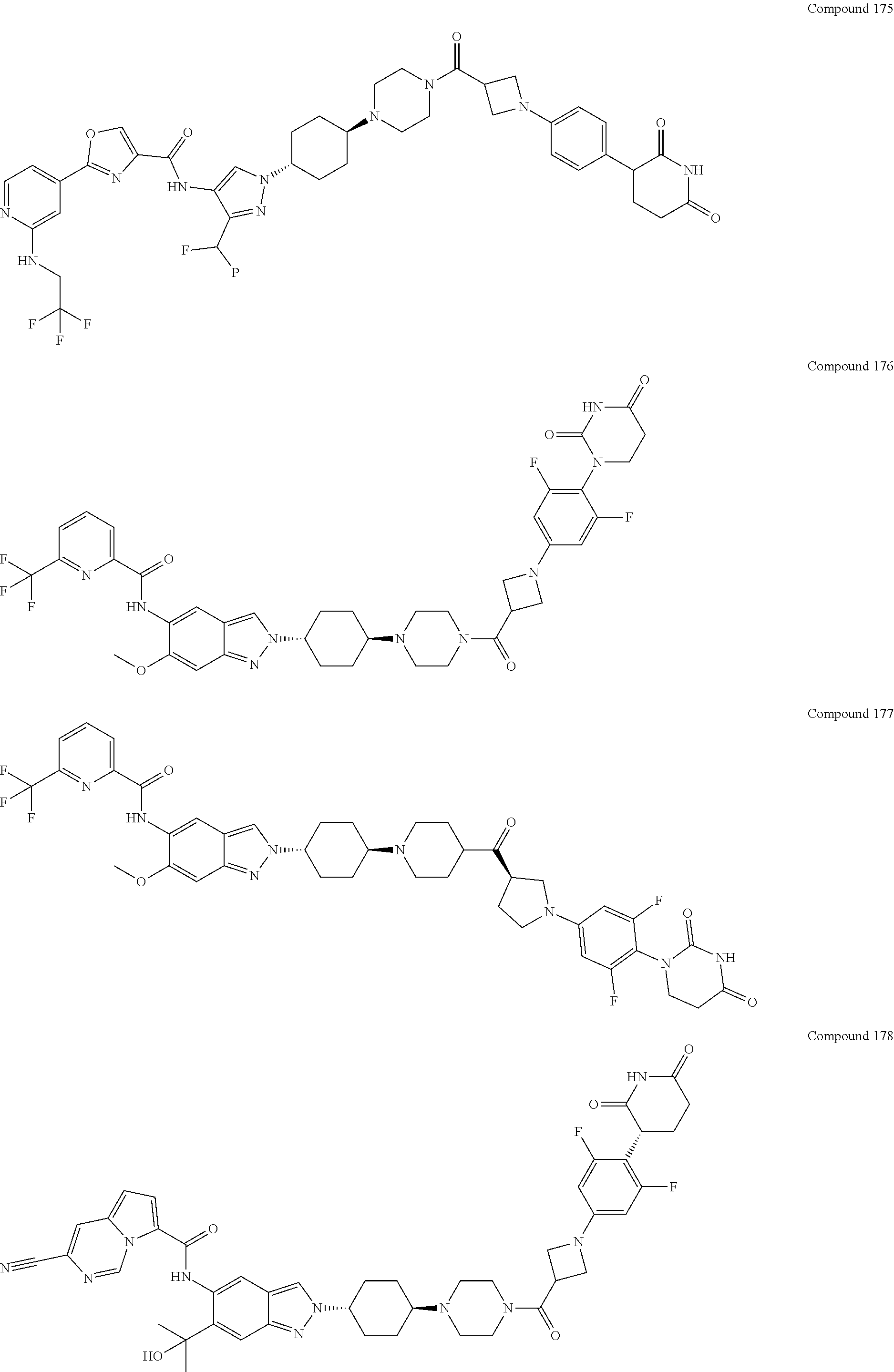

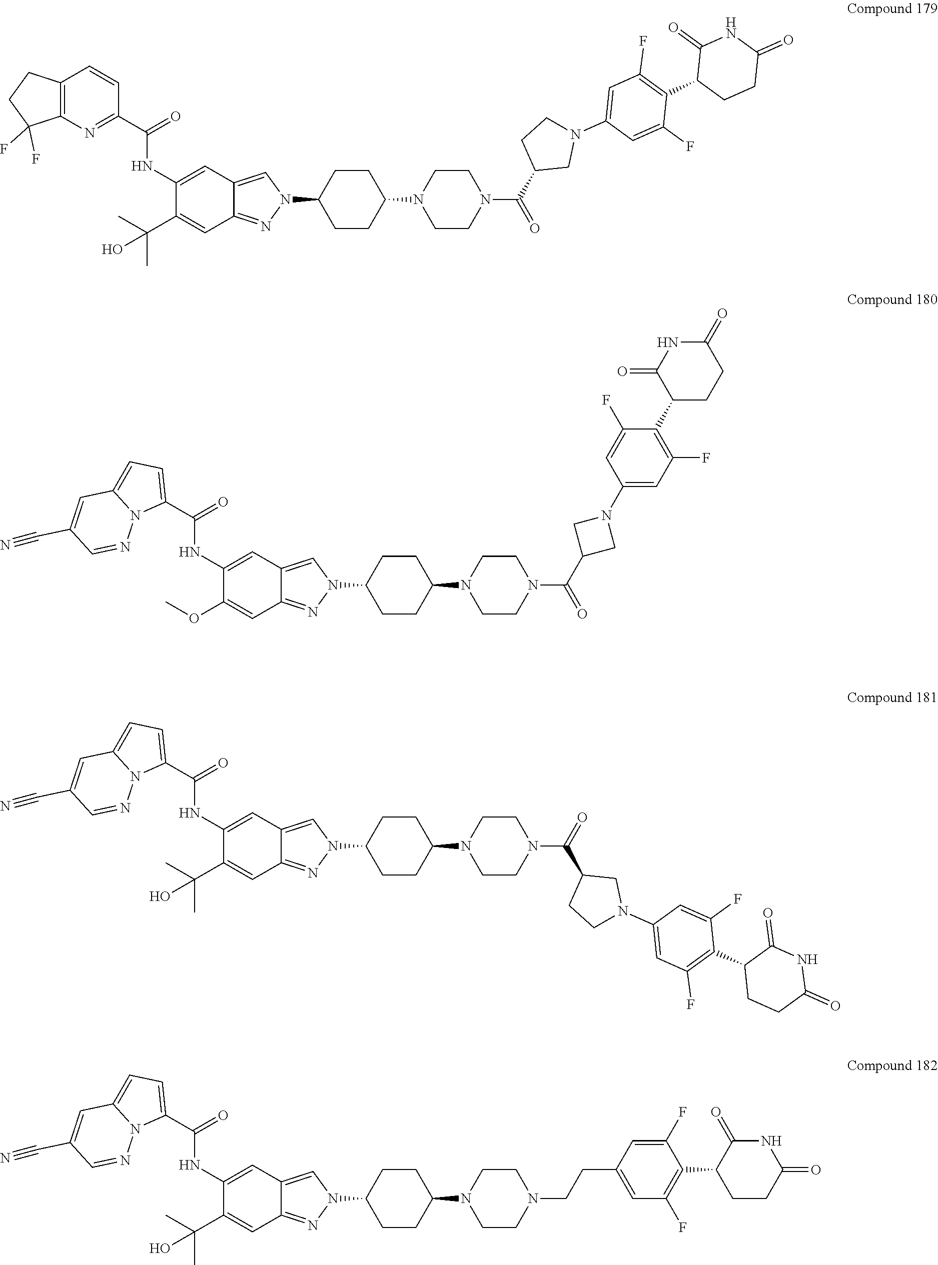 [SMILES: CC(C)(O)c1cc2nn([C@H]3CC[C@H](N4CCN(C(=O)[C@@H]5CCN(c6cc(F)c([C@H]7CCC(=O)NC7=O)c(F)c6)C5)CC4)CC3)cc2cc1NC(=O)c1ccc2c(n1)C(F)(F)CC2 ; COc1cc2nn([C@H]3CC[C@H](N4CCN(C(=O)C5CN(c6cc(F)c([C@H]7CCC(=O)NC7=O)c(F)c6)C5)CC4)CC3)cc2cc1NC(=O)c1ccc2cc(C#N)cnn12 ; CC(C)(O)c1cc2nn([C@H]3CC[C@H](N4CCN(C(=O)[C@@H]5CCN(c6cc(F)c([C@H]7CCC(=O)NC7=O)c(F)c6)C5)CC4)CC3)cc2cc1NC(=O)c1ccc2cc(C#N)cnn12 ; CC(C)(O)c1cc2nn([C@H]3CC[C@H](N4CCN(CCc5cc(F)c([C@H]6CCC(=O)NC6=O)c(F)c5)CC4)CC3)cc2cc1NC(=O)c1ccc2cc(C#N)cnn12]

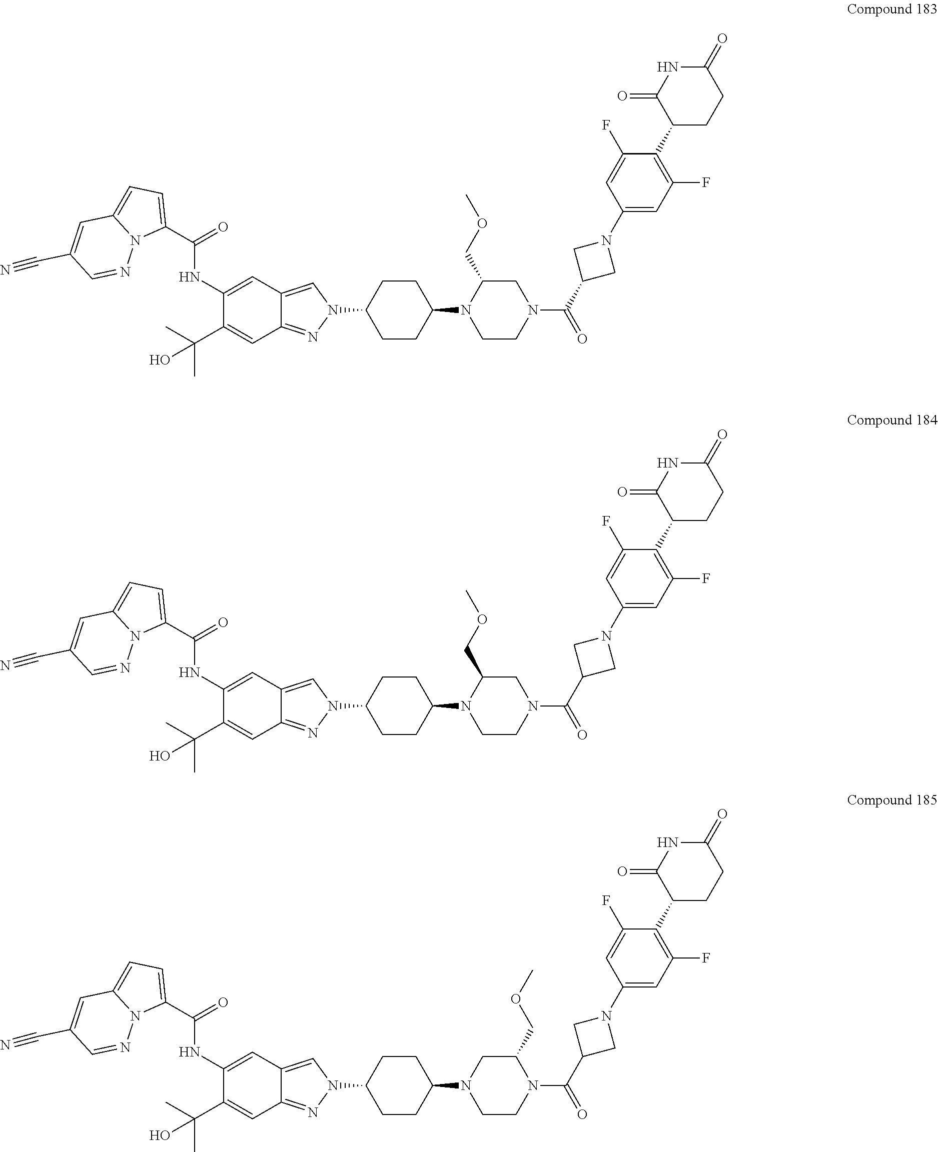

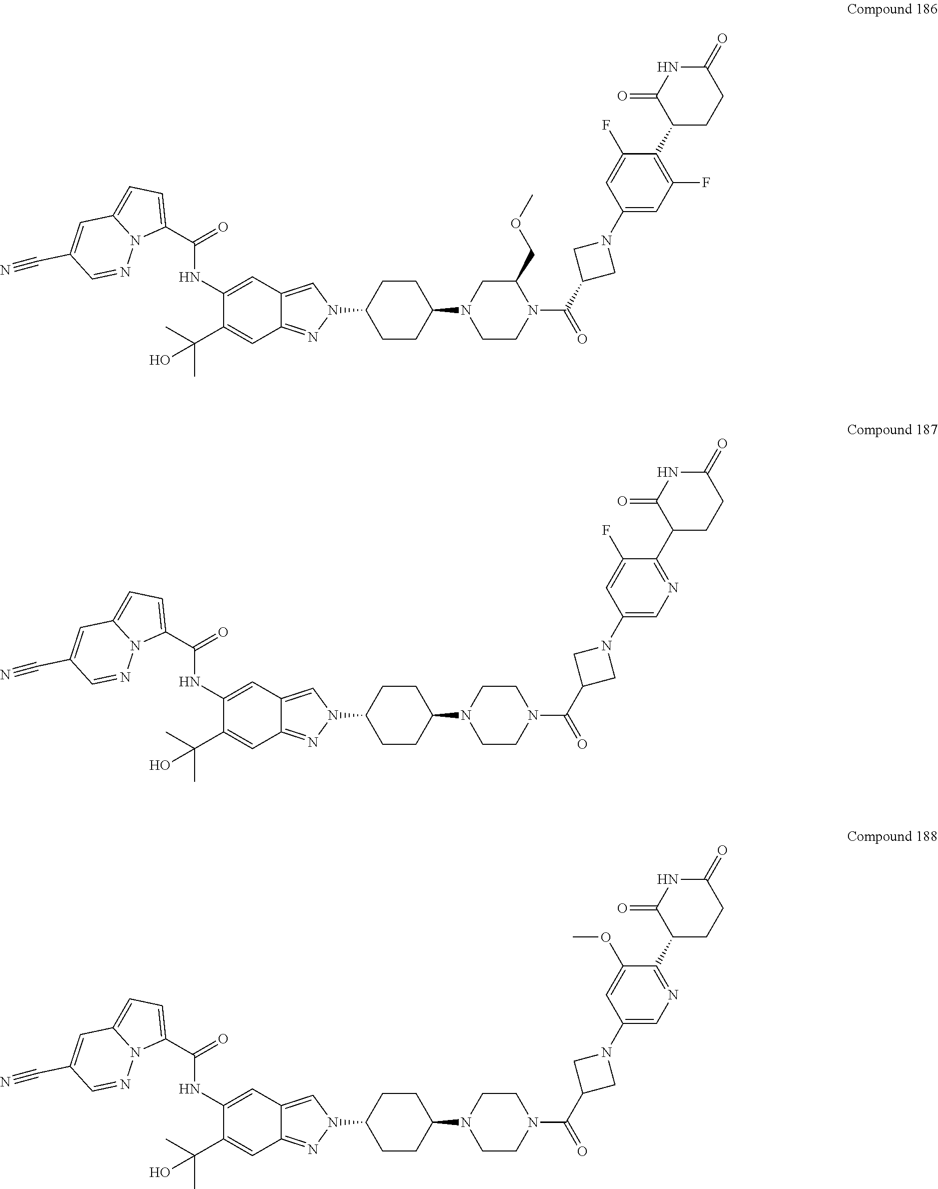

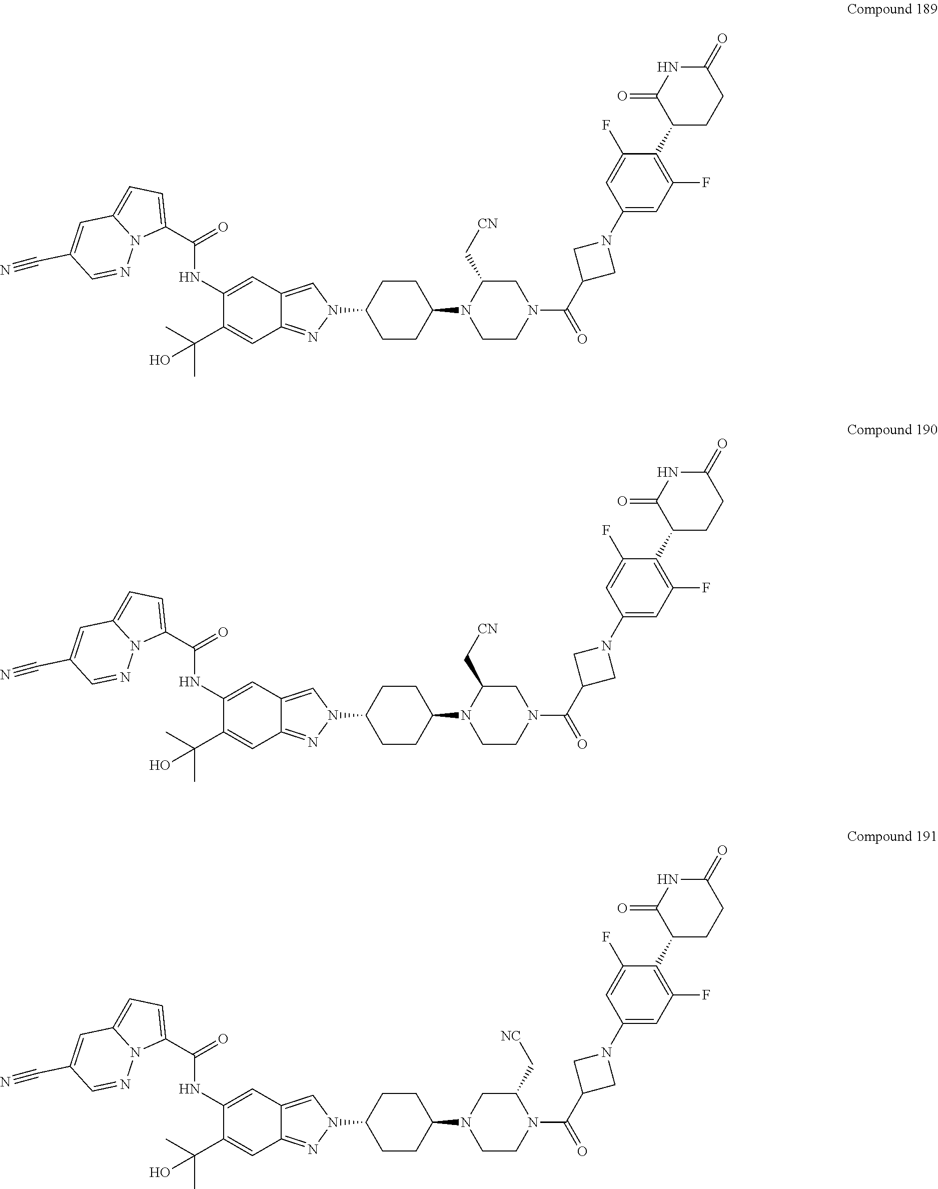 [SMILES: CC(C)(O)c1cc2nn([C@H]3CC[C@H](N4CCN(C(=O)C5CN(c6cc(F)c([C@H]7CCC(=O)NC7=O)c(F)c6)C5)C[C@H]4CC#N)CC3)cc2cc1NC(=O)c1ccc2cc(C#N)cnn12 ; CC(C)(O)c1cc2nn([C@H]3CC[C@H](N4CCN(C(=O)C5CN(c6cc(F)c([C@H]7CCC(=O)NC7=O)c(F)c6)C5)C[C@@H]4CC#N)CC3)cc2cc1NC(=O)c1ccc2cc(C#N)cnn12 ; CC(C)(O)c1cc2nn([C@H]3CC[C@H](N4CCN(C(=O)C5CN(c6cc(F)c([C@H]7CCC(=O)NC7=O)c(F)c6)C5)[C@@H](CC#N)C4)CC3)cc2cc1NC(=O)c1ccc2cc(C#N)cnn12]

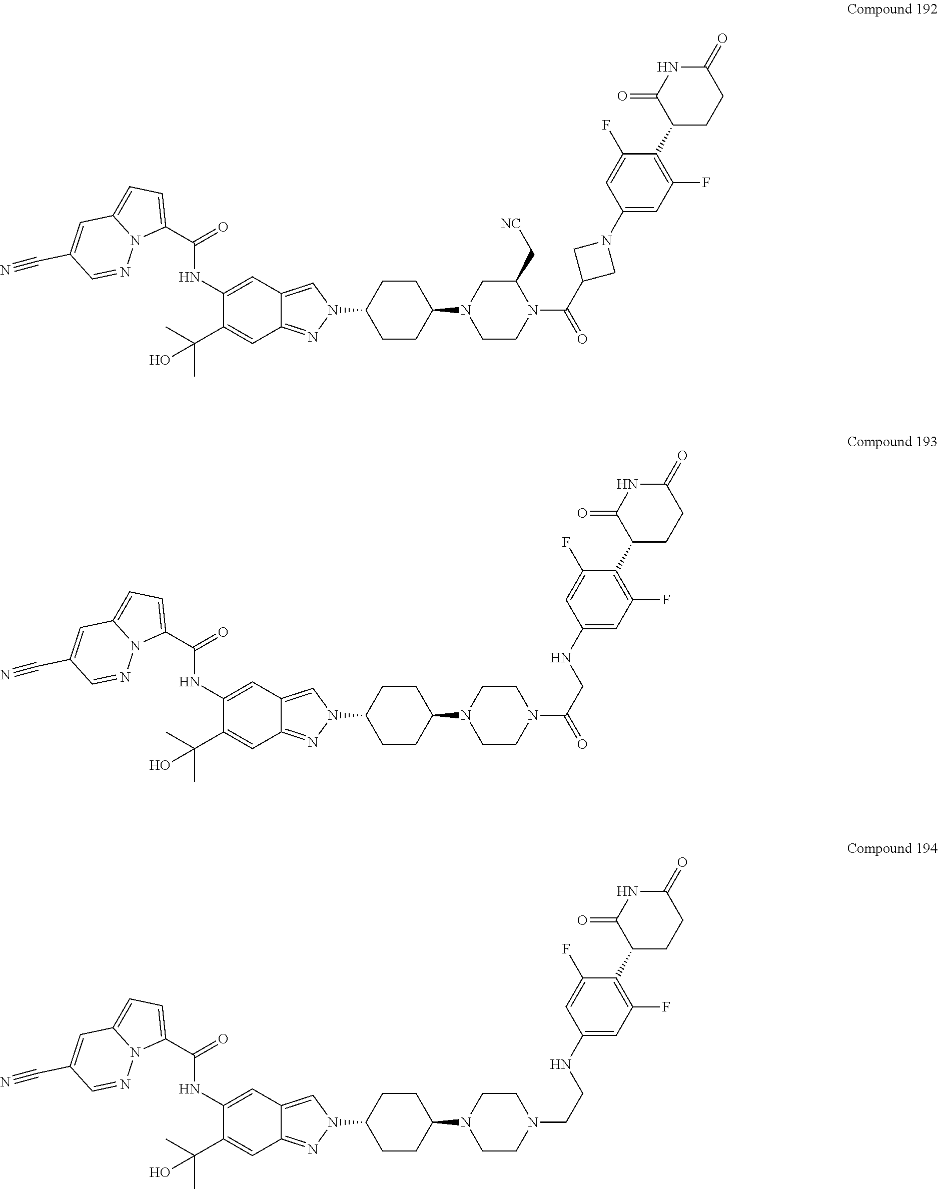 [SMILES: CC(C)(O)c1cc2nn([C@H]3CC[C@H](N4CCN(C(=O)C5CN(c6cc(F)c([C@H]7CCC(=O)NC7=O)c(F)c6)C5)[C@H](CC#N)C4)CC3)cc2cc1NC(=O)c1ccc2cc(C#N)cnn12 ; CC(C)(O)c1cc2nn([C@H]3CC[C@H](N4CCN(C(=O)CNc5cc(F)c([C@H]6CCC(=O)NC6=O)c(F)c5)CC4)CC3)cc2cc1NC(=O)c1ccc2cc(C#N)cnn12 ; CC(C)(O)c1cc2nn([C@H]3CC[C@H](N4CCN(CCNc5cc(F)c([C@H]6CCC(=O)NC6=O)c(F)c5)CC4)CC3)cc2cc1NC(=O)c1ccc2cc(C#N)cnn12]

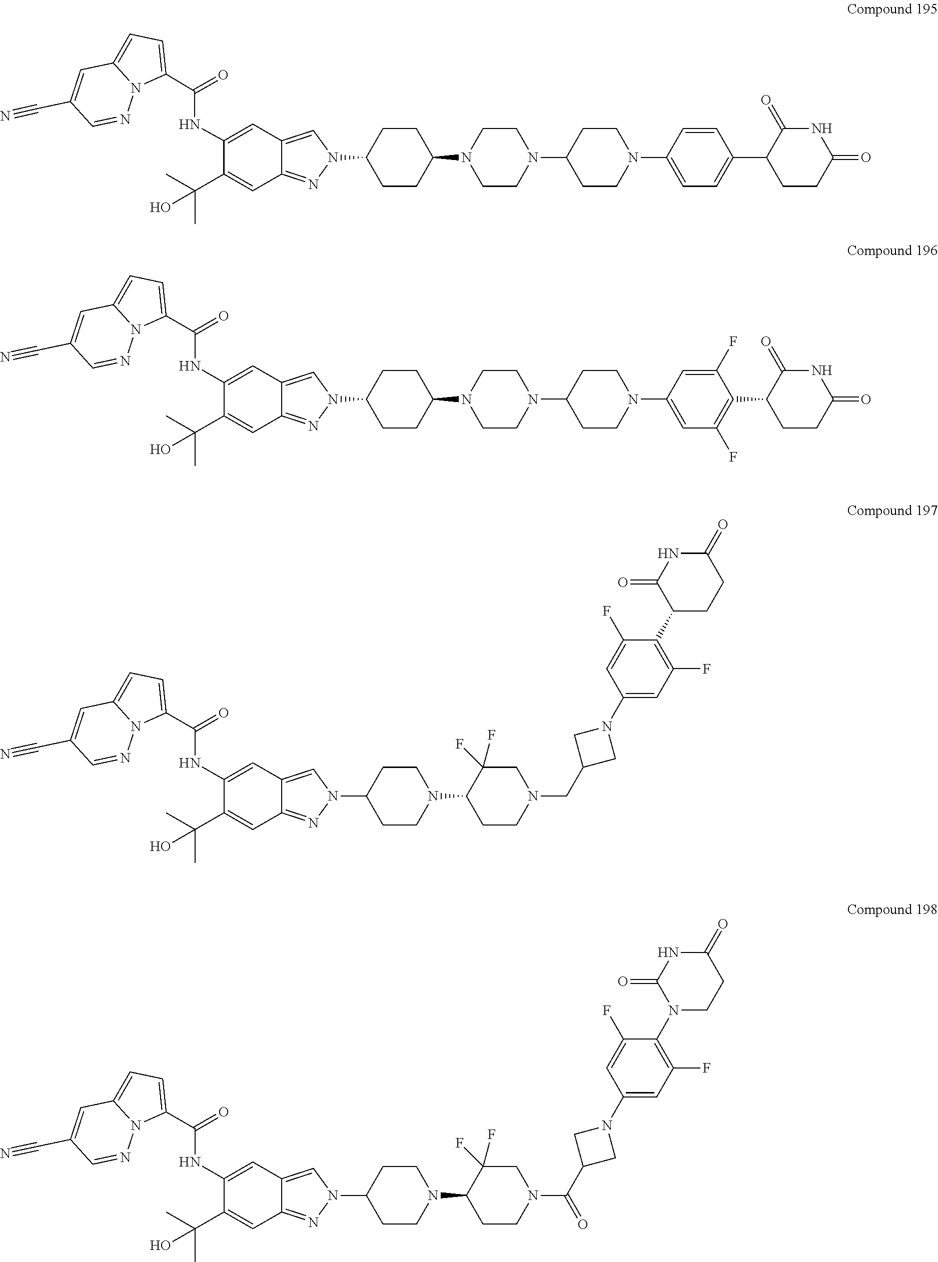 [SMILES: CC(C)(O)c1cc2nn([C@H]3CC[C@H](N4CCN(C5CCN(c6ccc(C7CCC(=O)NC7=O)cc6)CC5)CC4)CC3)cc2cc1NC(=O)c1ccc2cc(C#N)cnn12 ; CC(C)(O)c1cc2nn([C@H]3CC[C@H](N4CCN(C5CCN(c6cc(F)c([C@H]7CCC(=O)NC7=O)c(F)c6)CC5)CC4)CC3)cc2cc1NC(=O)c1ccc2cc(C#N)cnn12 ; CC(C)(O)c1cc2nn(C3CCN([C@H]4CCN(CC5CN(c6cc(F)c([C@H]7CCC(=O)NC7=O)c(F)c6)C5)CC4(F)F)CC3)cc2cc1NC(=O)c1ccc2cc(C#N)cnn12 ; CC(C)(O)c1cc2nn(C3CCN([C@@H]4CCN(C(=O)C5CN(c6cc(F)c(N7CCC(=O)NC7=O)c(F)c6)C5)CC4(F)F)CC3)cc2cc1NC(=O)c1ccc2cc(C#N)cnn12]

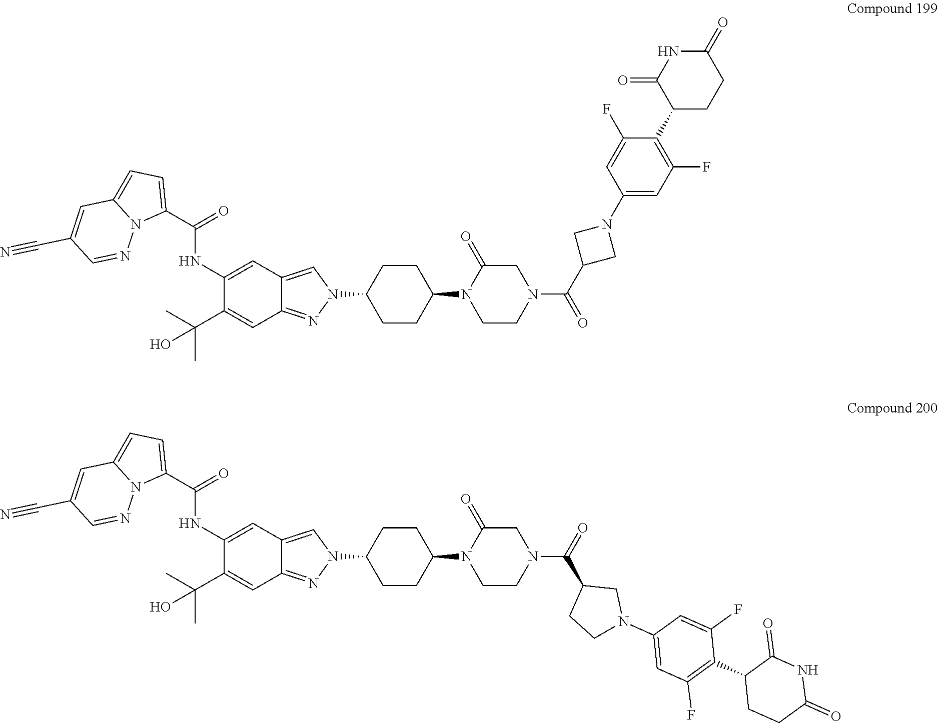 [SMILES: CC(C)(O)c1cc2nn([C@H]3CC[C@H](N4CCN(C(=O)C5CN(c6cc(F)c([C@H]7CCC(=O)NC7=O)c(F)c6)C5)CC4=O)CC3)cc2cc1NC(=O)c1ccc2cc(C#N)cnn12 ; CC(C)(O)c1cc2nn([C@H]3CC[C@H](N4CCN(C(=O)[C@@H]5CCN(c6cc(F)c([C@H]7CCC(=O)NC7=O)c(F)c6)C5)CC4=O)CC3)cc2cc1NC(=O)c1ccc2cc(C#N)cnn12]

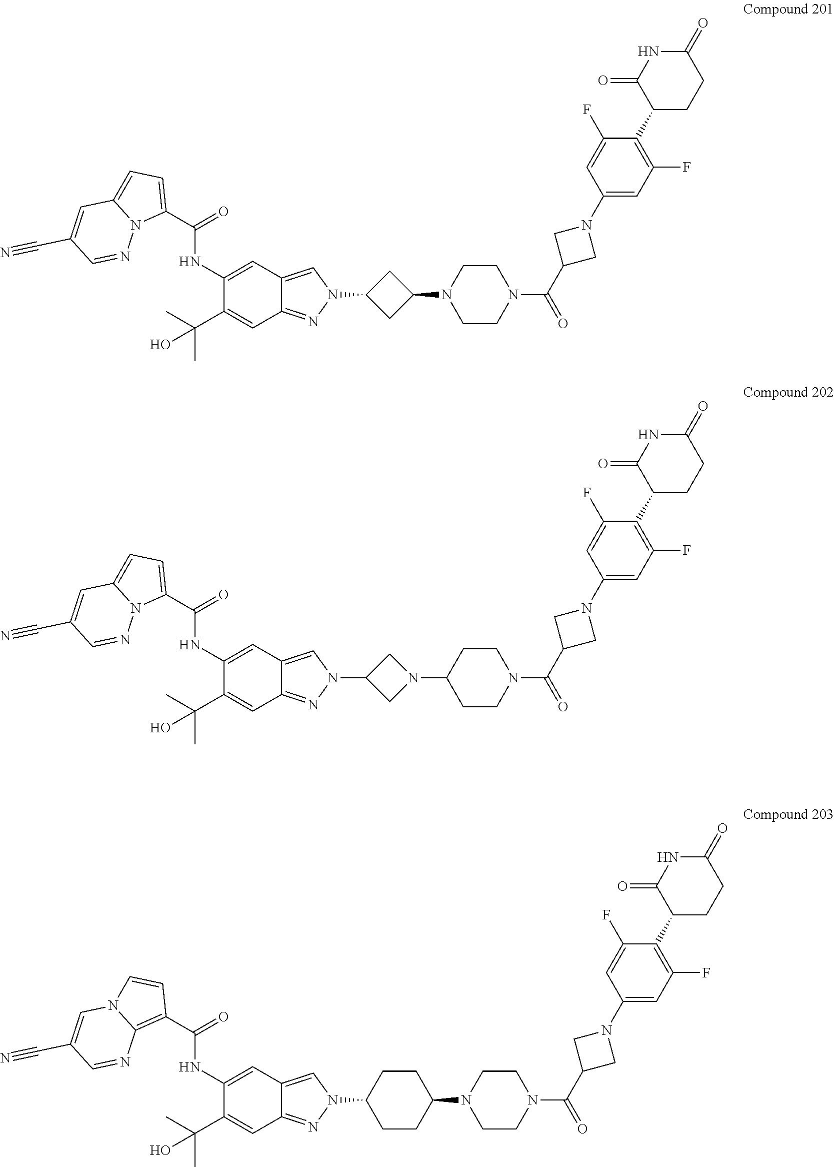 [SMILES: CC(C)(O)c1cc2nn([C@H]3C[C@H](N4CCN(C(=O)C5CN(c6cc(F)c([C@H]7CCC(=O)NC7=O)c(F)c6)C5)CC4)C3)cc2cc1NC(=O)c1ccc2cc(C#N)cnn12 ; CC(C)(O)c1cc2nn(C3CN(C4CCN(C(=O)C5CN(c6cc(F)c([C@H]7CCC(=O)NC7=O)c(F)c6)C5)CC4)C3)cc2cc1NC(=O)c1ccc2cc(C#N)cnn12 ; CC(C)(O)c1cc2nn([C@H]3CC[C@H](N4CCN(C(=O)C5CN(c6cc(F)c([C@H]7CCC(=O)NC7=O)c(F)c6)C5)CC4)CC3)cc2cc1NC(=O)c1ccn2cc(C#N)cnc12]

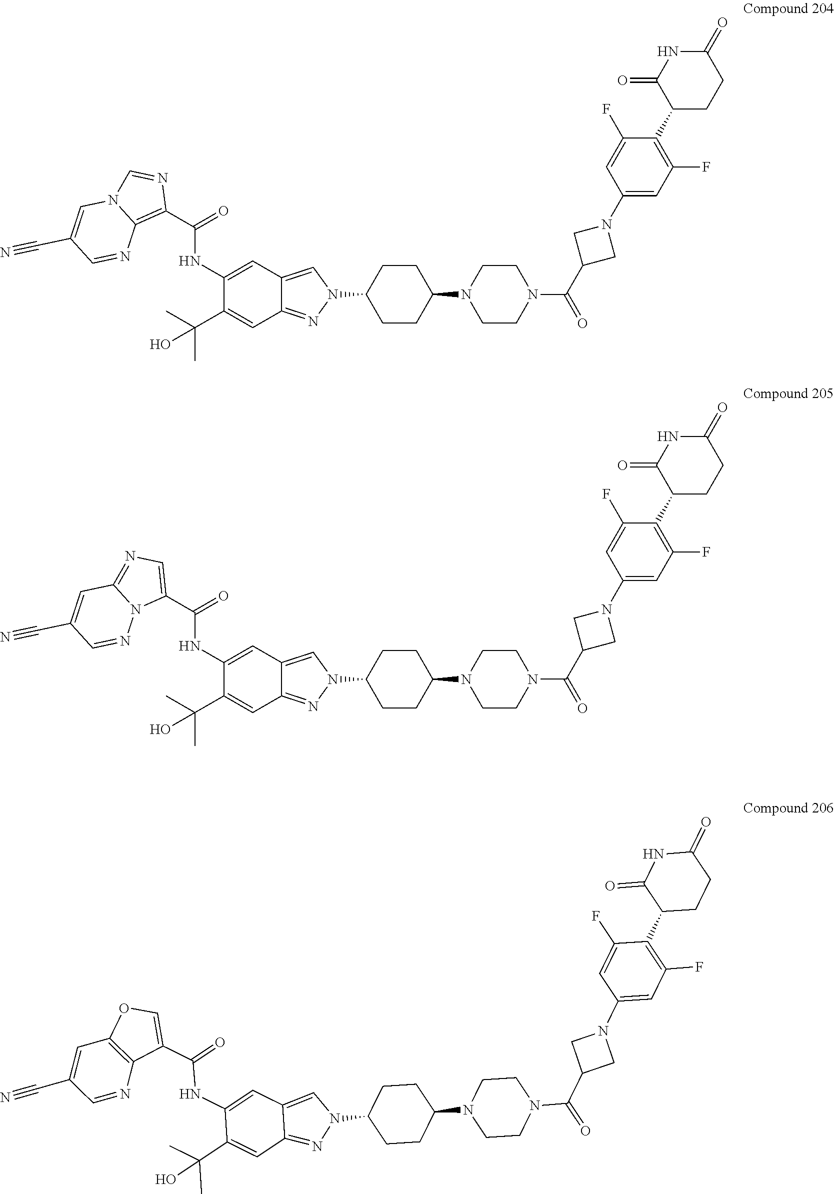 [SMILES: CC(C)(O)c1cc2nn([C@H]3CC[C@H](N4CCN(C(=O)C5CN(c6cc(F)c([C@H]7CCC(=O)NC7=O)c(F)c6)C5)CC4)CC3)cc2cc1NC(=O)c1ncn2cc(C#N)cnc12 ; CC(C)(O)c1cc2nn([C@H]3CC[C@H](N4CCN(C(=O)C5CN(c6cc(F)c([C@H]7CCC(=O)NC7=O)c(F)c6)C5)CC4)CC3)cc2cc1NC(=O)c1cnc2cc(C#N)cnn12 ; CC(C)(O)c1cc2nn([C@H]3CC[C@H](N4CCN(C(=O)C5CN(c6cc(F)c([C@H]7CCC(=O)NC7=O)c(F)c6)C5)CC4)CC3)cc2cc1NC(=O)c1coc2cc(C#N)cnc12]

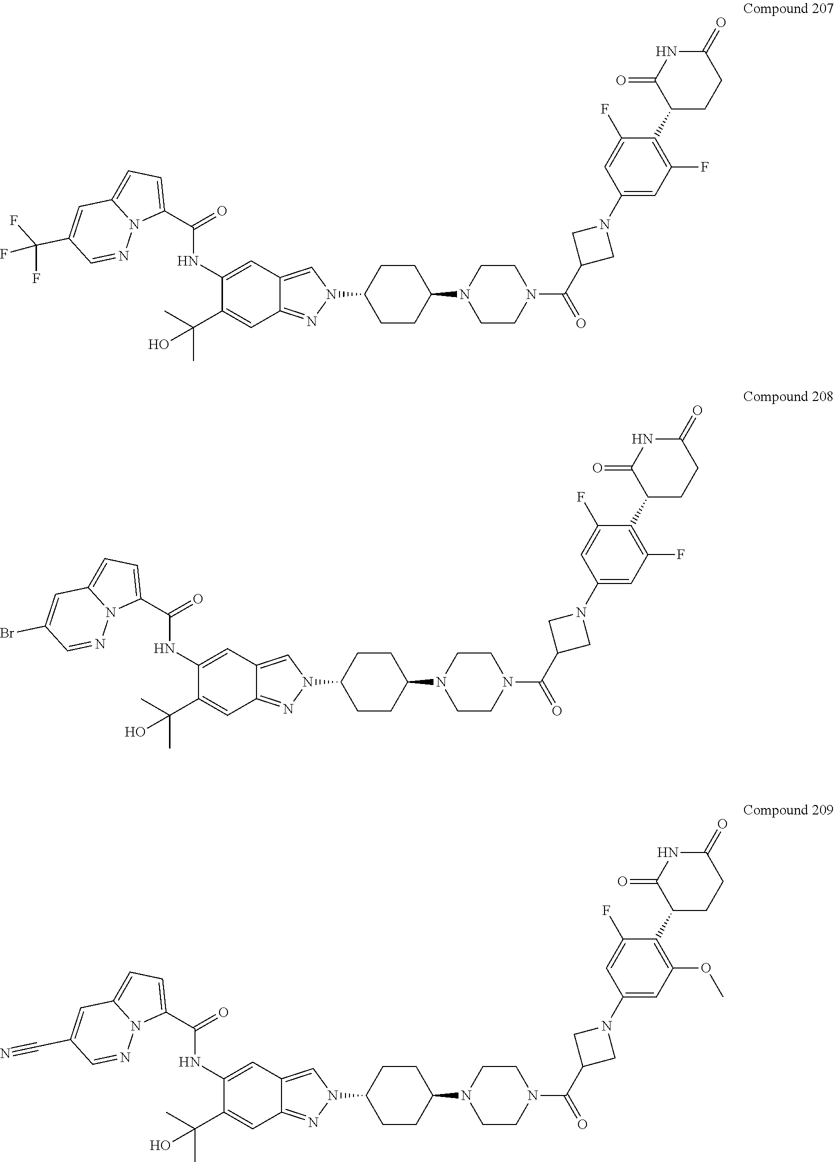 [SMILES: CC(C)(O)c1cc2nn([C@H]3CC[C@H](N4CCN(C(=O)C5CN(c6cc(F)c([C@H]7CCC(=O)NC7=O)c(F)c6)C5)CC4)CC3)cc2cc1NC(=O)c1ccc2cc(C(F)(F)F)cnn12 ; CC(C)(O)c1cc2nn([C@H]3CC[C@H](N4CCN(C(=O)C5CN(c6cc(F)c([C@H]7CCC(=O)NC7=O)c(F)c6)C5)CC4)CC3)cc2cc1NC(=O)c1ccc2cc(Br)cnn12 ; COc1cc(N2CC(C(=O)N3CCN([C@H]4CC[C@H](n5cc6cc(NC(=O)c7ccc8cc(C#N)cnn78)c(C(C)(C)O)cc6n5)CC4)CC3)C2)cc(F)c1[C@H]1CCC(=O)NC1=O]

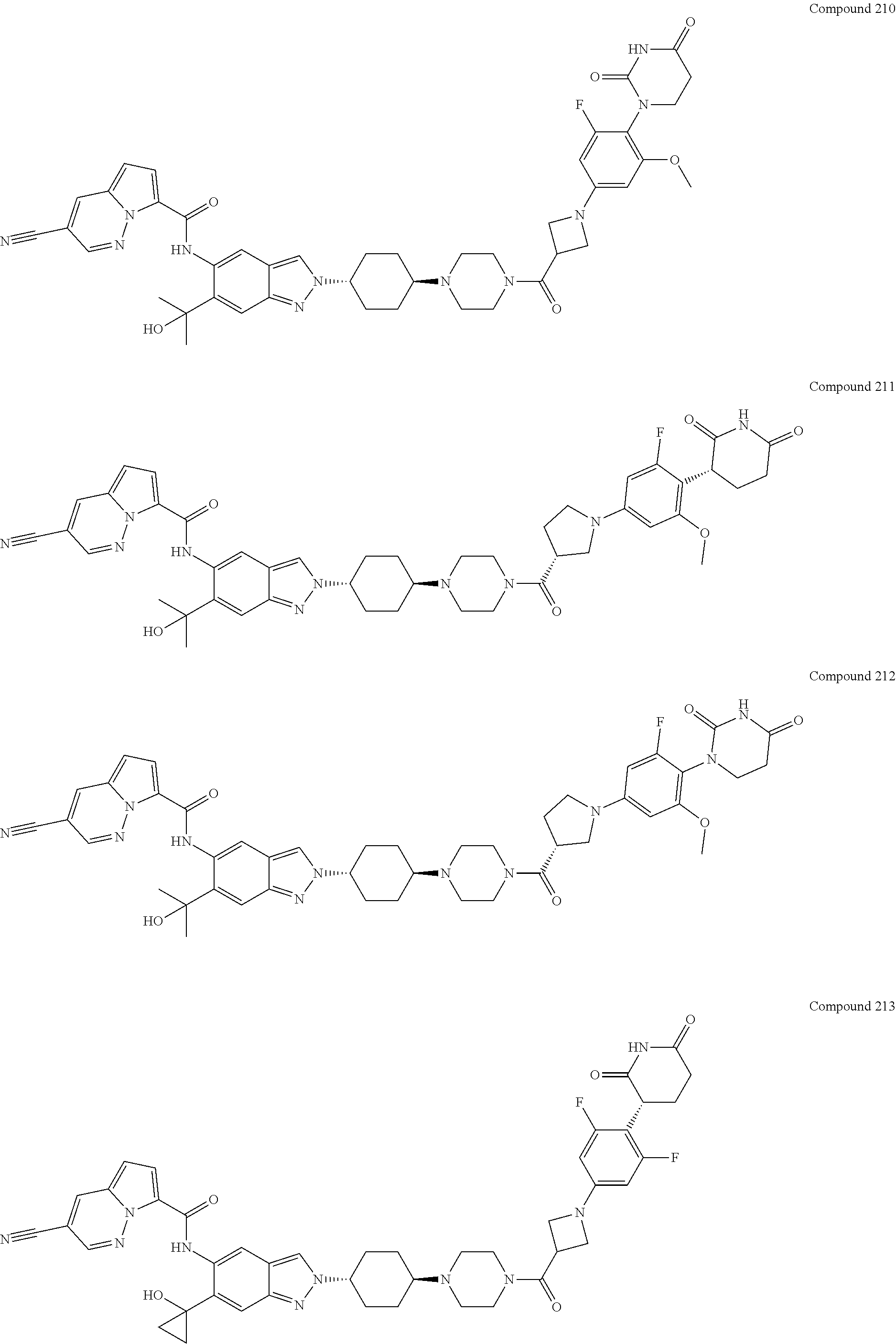

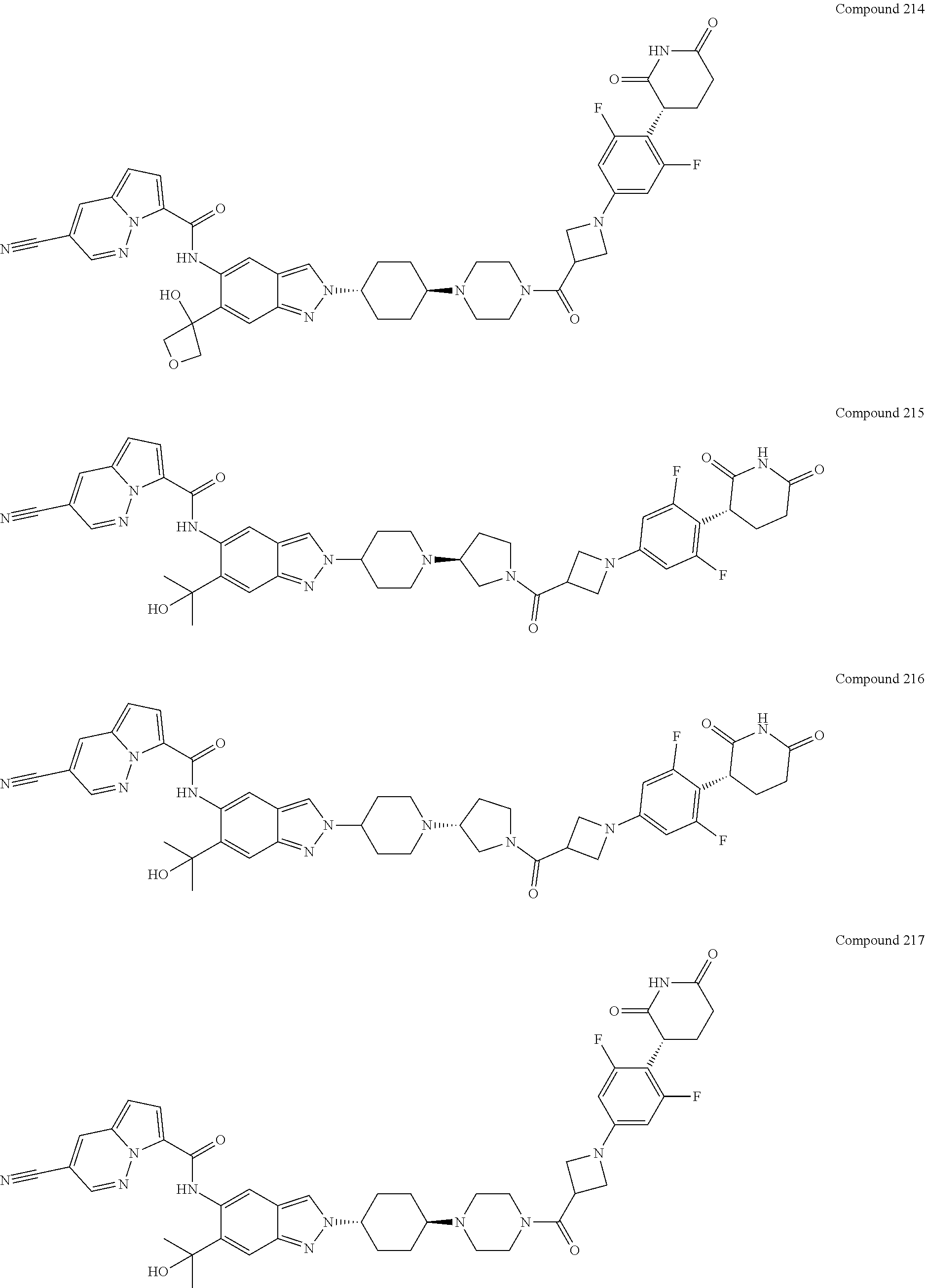 [SMILES: N#Cc1cnn2c(C(=O)Nc3cc4cn([C@H]5CC[C@H](N6CCN(C(=O)C7CN(c8cc(F)c([C@H]9CCC(=O)NC9=O)c(F)c8)C7)CC6)CC5)nc4cc3C3(O)COC3)ccc2c1 ; CC(C)(O)c1cc2nn(C3CCN([C@H]4CCN(C(=O)C5CN(c6cc(F)c([C@H]7CCC(=O)NC7=O)c(F)c6)C5)C4)CC3)cc2cc1NC(=O)c1ccc2cc(C#N)cnn12 ; CC(C)(O)c1cc2nn(C3CCN([C@@H]4CCN(C(=O)C5CN(c6cc(F)c([C@H]7CCC(=O)NC7=O)c(F)c6)C5)C4)CC3)cc2cc1NC(=O)c1ccc2cc(C#N)cnn12 ; CC(C)(O)c1cc2nn([C@H]3CC[C@H](N4CCN(C(=O)C5CN(c6cc(F)c([C@H]7CCC(=O)NC7=O)c(F)c6)C5)CC4)CC3)cc2cc1NC(=O)c1ccc2cc(C#N)cnn12]

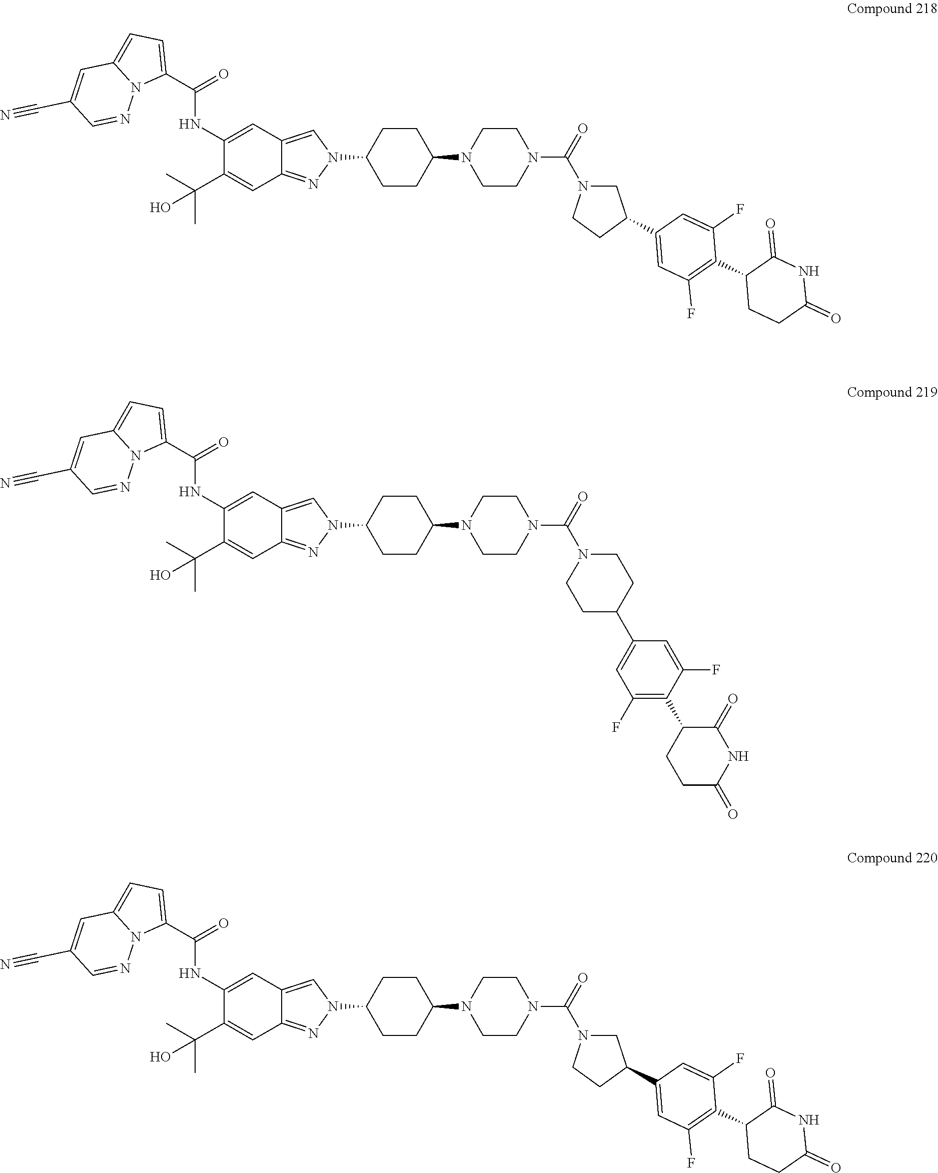 [SMILES: CC(C)(O)c1cc2nn([C@H]3CC[C@H](N4CCN(C(=O)N5CC[C@@H](c6cc(F)c([C@H]7CCC(=O)NC7=O)c(F)c6)C5)CC4)CC3)cc2cc1NC(=O)c1ccc2cc(C#N)cnn12 ; CC(C)(O)c1cc2nn([C@H]3CC[C@H](N4CCN(C(=O)N5CCC(c6cc(F)c([C@H]7CCC(=O)NC7=O)c(F)c6)CC5)CC4)CC3)cc2cc1NC(=O)c1ccc2cc(C#N)cnn12 ; CC(C)(O)c1cc2nn([C@H]3CC[C@H](N4CCN(C(=O)N5CC[C@H](c6cc(F)c([C@H]7CCC(=O)NC7=O)c(F)c6)C5)CC4)CC3)cc2cc1NC(=O)c1ccc2cc(C#N)cnn12]

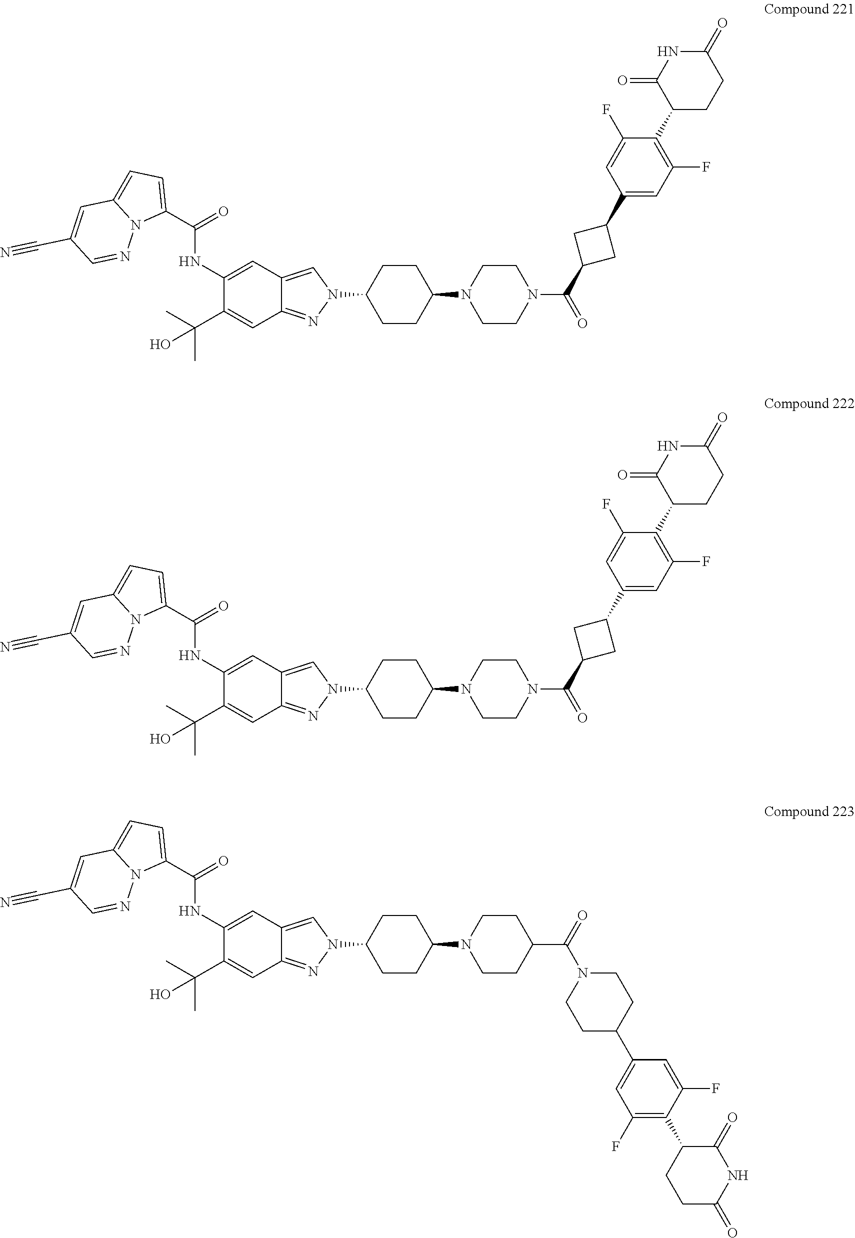 [SMILES: CC(C)(O)c1cc2nn([C@H]3CC[C@H](N4CCN(C(=O)[C@H]5C[C@@H](c6cc(F)c([C@H]7CCC(=O)NC7=O)c(F)c6)C5)CC4)CC3)cc2cc1NC(=O)c1ccc2cc(C#N)cnn12 ; CC(C)(O)c1cc2nn([C@H]3CC[C@H](N4CCN(C(=O)[C@H]5C[C@H](c6cc(F)c([C@H]7CCC(=O)NC7=O)c(F)c6)C5)CC4)CC3)cc2cc1NC(=O)c1ccc2cc(C#N)cnn12 ; CC(C)(O)c1cc2nn([C@H]3CC[C@H](N4CCC(C(=O)N5CCC(c6cc(F)c([C@H]7CCC(=O)NC7=O)c(F)c6)CC5)CC4)CC3)cc2cc1NC(=O)c1ccc2cc(C#N)cnn12]

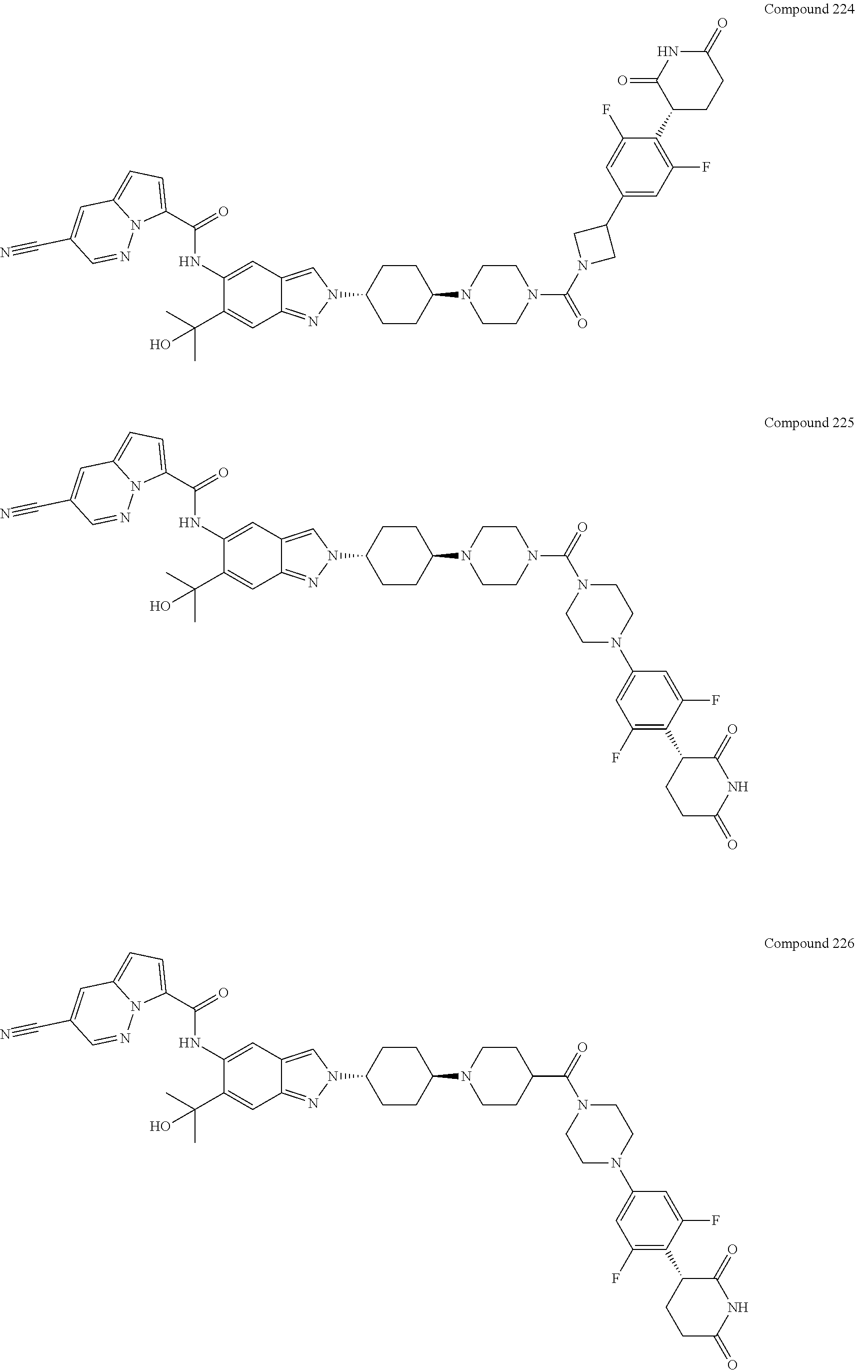 [SMILES: CC(C)(O)c1cc2nn([C@H]3CC[C@H](N4CCN(C(=O)N5CC(c6cc(F)c([C@H]7CCC(=O)NC7=O)c(F)c6)C5)CC4)CC3)cc2cc1NC(=O)c1ccc2cc(C#N)cnn12 ; CC(C)(O)c1cc2nn([C@H]3CC[C@H](N4CCN(C(=O)N5CCN(c6cc(F)c([C@H]7CCC(=O)NC7=O)c(F)c6)CC5)CC4)CC3)cc2cc1NC(=O)c1ccc2cc(C#N)cnn12 ; CC(C)(O)c1cc2nn([C@H]3CC[C@H](N4CCC(C(=O)N5CCN(c6cc(F)c([C@H]7CCC(=O)NC7=O)c(F)c6)CC5)CC4)CC3)cc2cc1NC(=O)c1ccc2cc(C#N)cnn12]

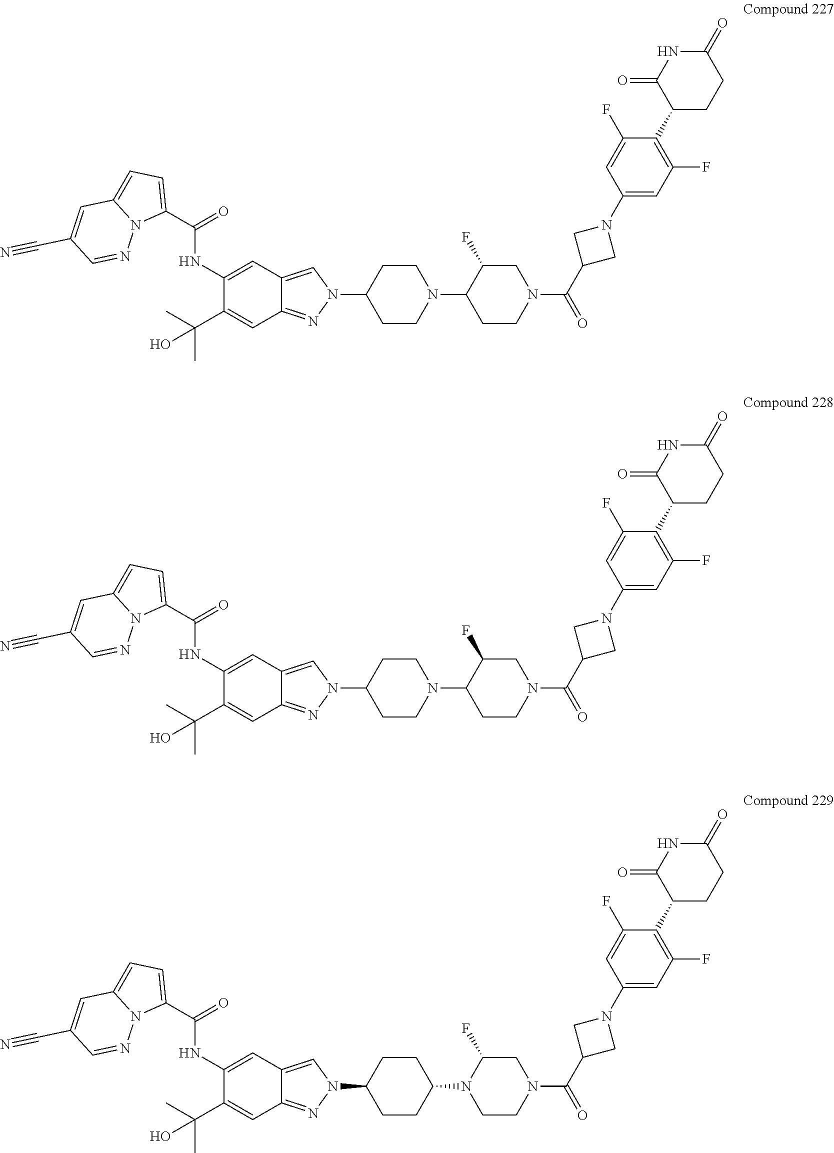 [SMILES: CC(C)(O)c1cc2nn(C3CCN(C4CCN(C(=O)C5CN(c6cc(F)c([C@H]7CCC(=O)NC7=O)c(F)c6)C5)C[C@H]4F)CC3)cc2cc1NC(=O)c1ccc2cc(C#N)cnn12 ; CC(C)(O)c1cc2nn(C3CCN(C4CCN(C(=O)C5CN(c6cc(F)c([C@H]7CCC(=O)NC7=O)c(F)c6)C5)C[C@@H]4F)CC3)cc2cc1NC(=O)c1ccc2cc(C#N)cnn12 ; CC(C)(O)c1cc2nn([C@H]3CC[C@H](N4CCN(C(=O)C5CN(c6cc(F)c([C@H]7CCC(=O)NC7=O)c(F)c6)C5)C[C@H]4F)CC3)cc2cc1NC(=O)c1ccc2cc(C#N)cnn12]

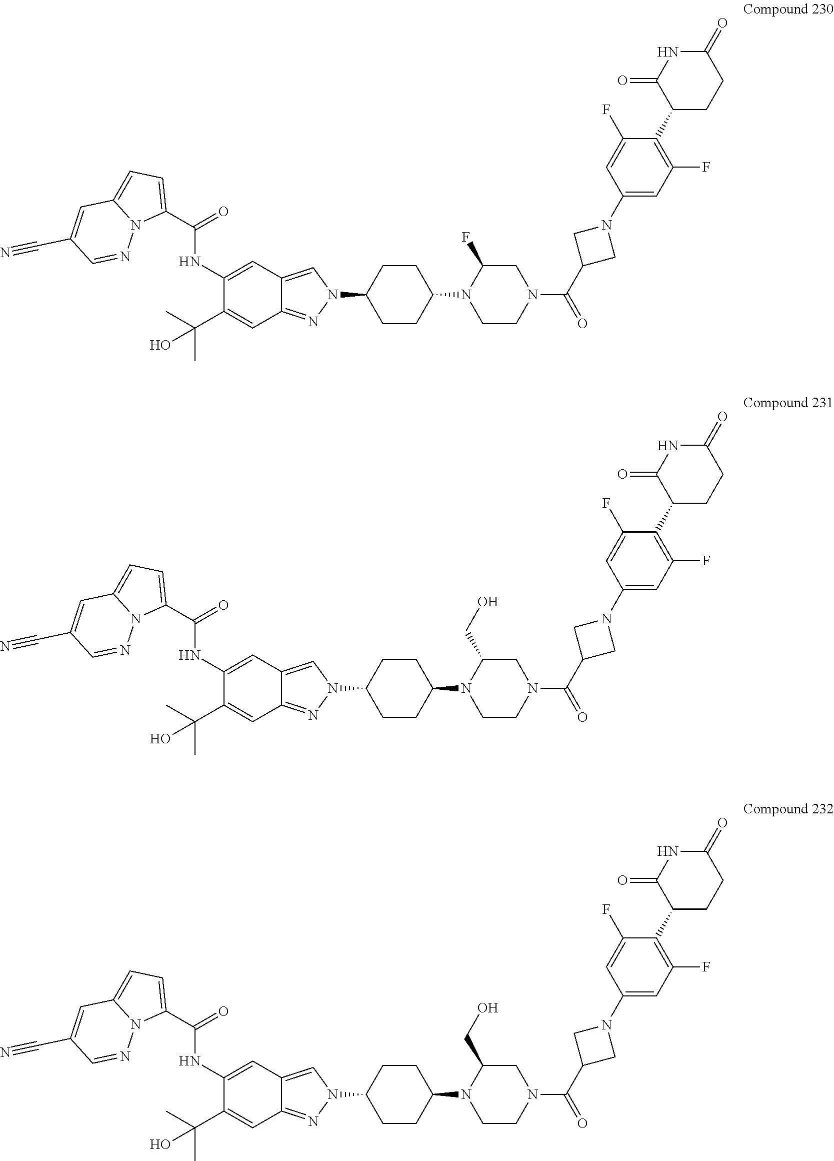 [SMILES: CC(C)(O)c1cc2nn([C@H]3CC[C@H](N4CCN(C(=O)C5CN(c6cc(F)c([C@H]7CCC(=O)NC7=O)c(F)c6)C5)C[C@@H]4F)CC3)cc2cc1NC(=O)c1ccc2cc(C#N)cnn12 ; CC(C)(O)c1cc2nn([C@H]3CC[C@H](N4CCN(C(=O)C5CN(c6cc(F)c([C@H]7CCC(=O)NC7=O)c(F)c6)C5)C[C@H]4CO)CC3)cc2cc1NC(=O)c1ccc2cc(C#N)cnn12 ; CC(C)(O)c1cc2nn([C@H]3CC[C@H](N4CCN(C(=O)C5CN(c6cc(F)c([C@H]7CCC(=O)NC7=O)c(F)c6)C5)C[C@@H]4CO)CC3)cc2cc1NC(=O)c1ccc2cc(C#N)cnn12]

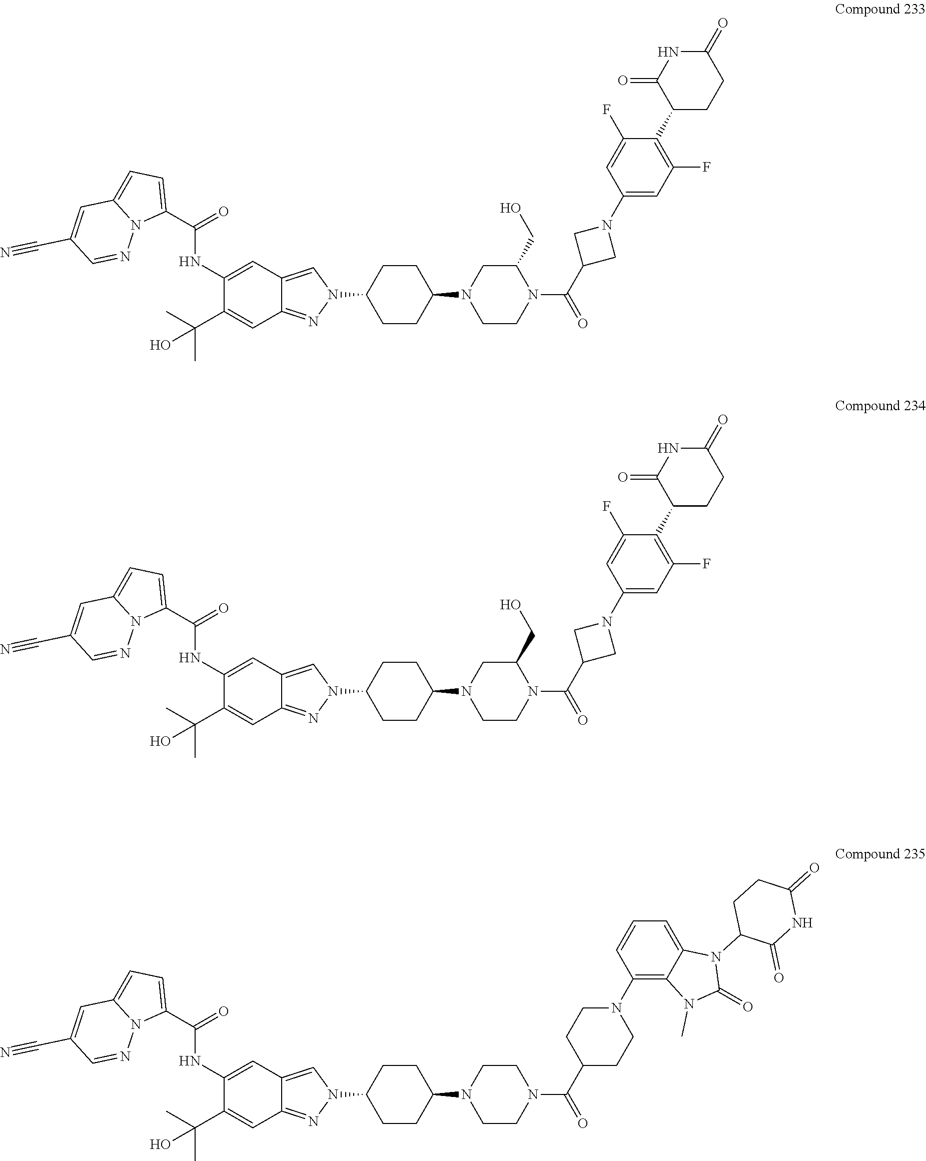 [SMILES: CC(C)(O)c1cc2nn([C@H]3CC[C@H](N4CCN(C(=O)C5CN(c6cc(F)c([C@H]7CCC(=O)NC7=O)c(F)c6)C5)[C@@H](CO)C4)CC3)cc2cc1NC(=O)c1ccc2cc(C#N)cnn12 ; CC(C)(O)c1cc2nn([C@H]3CC[C@H](N4CCN(C(=O)C5CN(c6cc(F)c([C@H]7CCC(=O)NC7=O)c(F)c6)C5)[C@H](CO)C4)CC3)cc2cc1NC(=O)c1ccc2cc(C#N)cnn12 ; Cn1c(=O)n(C2CCC(=O)NC2=O)c2cccc(N3CCC(C(=O)N4CCN([C@H]5CC[C@H](n6cc7cc(NC(=O)c8ccc9cc(C#N)cnn89)c(C(C)(C)O)cc7n6)CC5)CC4)CC3)c21]

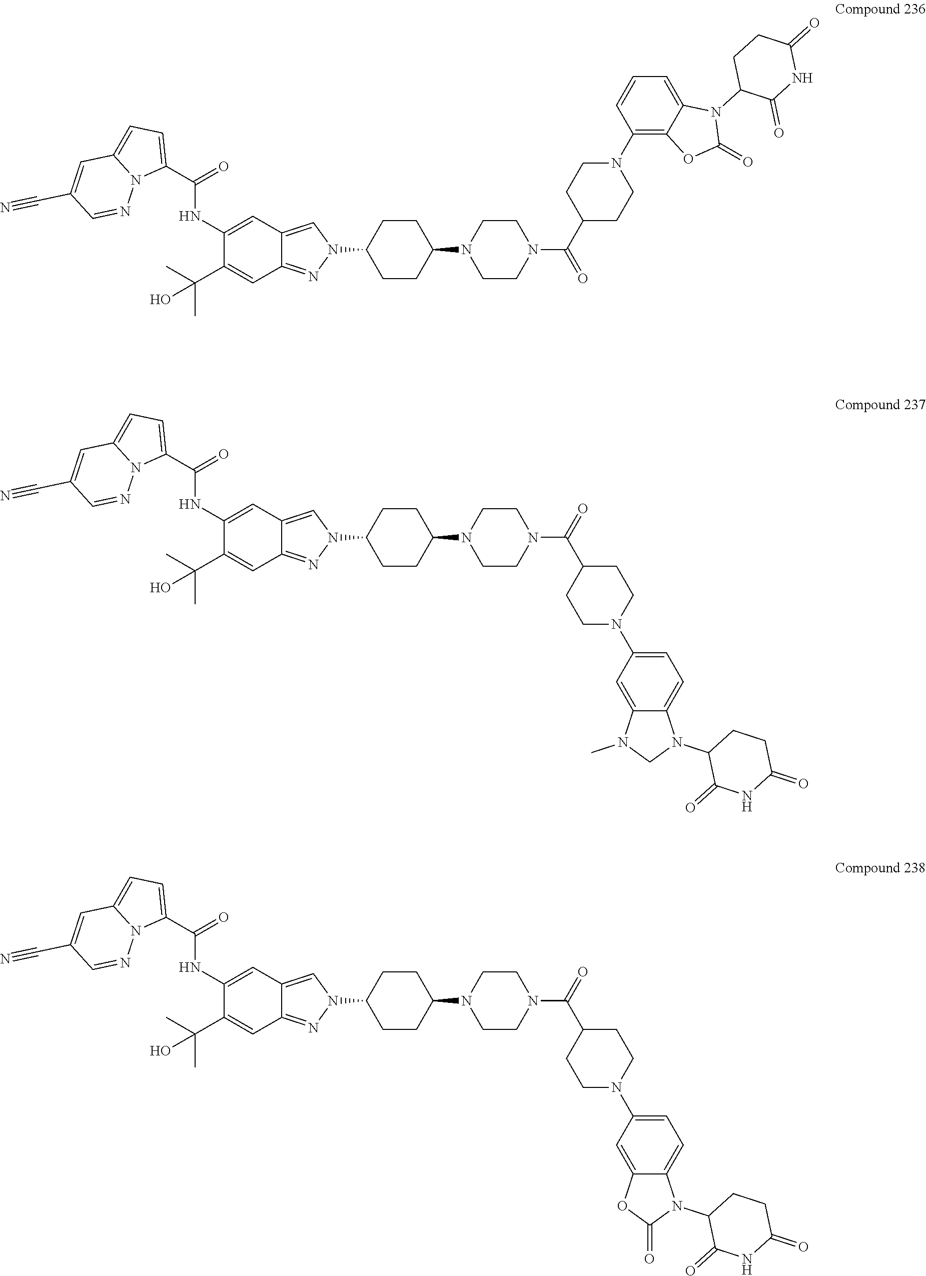 [SMILES: CC(C)(O)c1cc2nn([C@H]3CC[C@H](N4CCN(C(=O)C5CCN(c6cccc7c6oc(=O)n7C6CCC(=O)NC6=O)CC5)CC4)CC3)cc2cc1NC(=O)c1ccc2cc(C#N)cnn12 ; CN1CN(C2CCC(=O)NC2=O)c2ccc(N3CCC(C(=O)N4CCN([C@H]5CC[C@H](n6cc7cc(NC(=O)c8ccc9cc(C#N)cnn89)c(C(C)(C)O)cc7n6)CC5)CC4)CC3)cc21 ; CC(C)(O)c1cc2nn([C@H]3CC[C@H](N4CCN(C(=O)C5CCN(c6ccc7c(c6)oc(=O)n7C6CCC(=O)NC6=O)CC5)CC4)CC3)cc2cc1NC(=O)c1ccc2cc(C#N)cnn12]

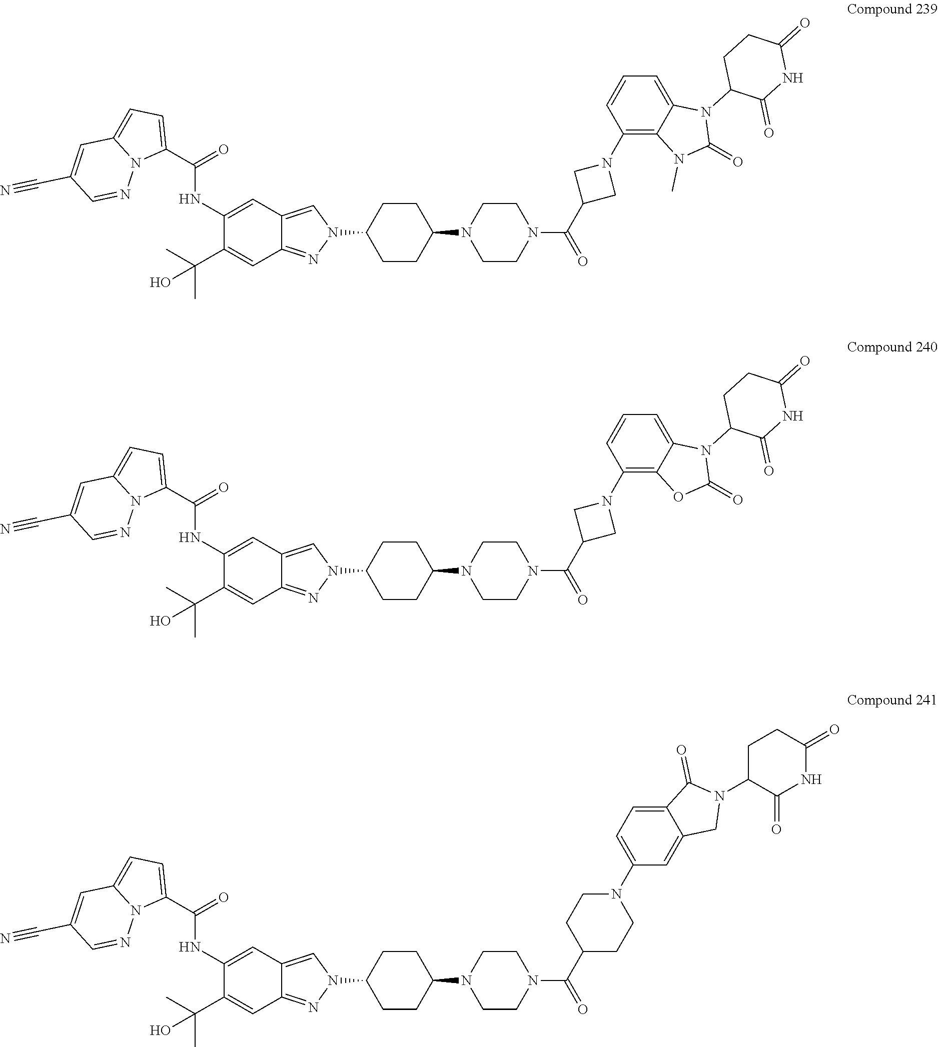 [SMILES: Cn1c(=O)n(C2CCC(=O)NC2=O)c2cccc(N3CC(C(=O)N4CCN([C@H]5CC[C@H](n6cc7cc(NC(=O)c8ccc9cc(C#N)cnn89)c(C(C)(C)O)cc7n6)CC5)CC4)C3)c21 ; CC(C)(O)c1cc2nn([C@H]3CC[C@H](N4CCN(C(=O)C5CN(c6cccc7c6oc(=O)n7C6CCC(=O)NC6=O)C5)CC4)CC3)cc2cc1NC(=O)c1ccc2cc(C#N)cnn12 ; CC(C)(O)c1cc2nn([C@H]3CC[C@H](N4CCN(C(=O)C5CCN(c6ccc7c(c6)CN(C6CCC(=O)NC6=O)C7=O)CC5)CC4)CC3)cc2cc1NC(=O)c1ccc2cc(C#N)cnn12]

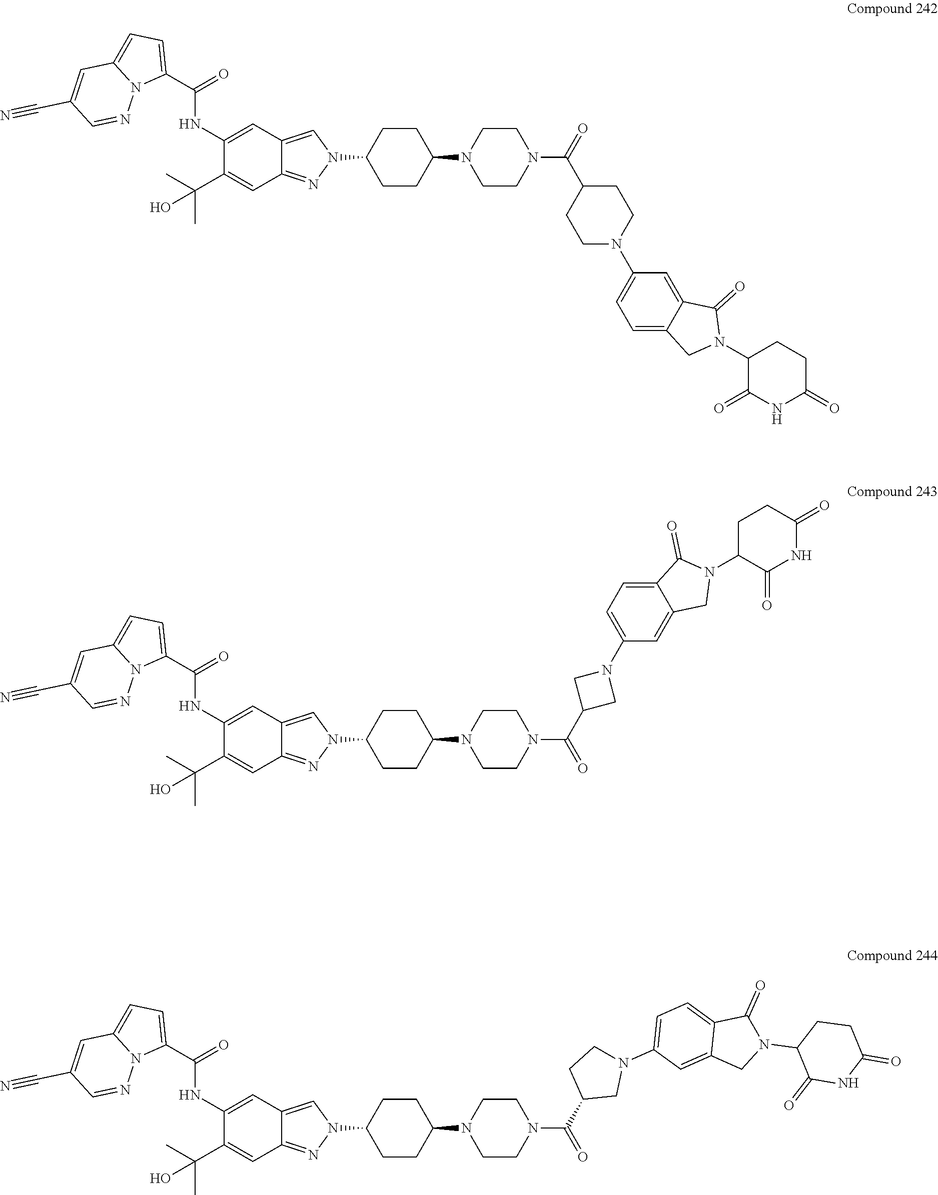 [SMILES: CC(C)(O)c1cc2nn([C@H]3CC[C@H](N4CCN(C(=O)C5CCN(c6ccc7c(c6)C(=O)N(C6CCC(=O)NC6=O)C7)CC5)CC4)CC3)cc2cc1NC(=O)c1ccc2cc(C#N)cnn12 ; CC(C)(O)c1cc2nn([C@H]3CC[C@H](N4CCN(C(=O)C5CN(c6ccc7c(c6)CN(C6CCC(=O)NC6=O)C7=O)C5)CC4)CC3)cc2cc1NC(=O)c1ccc2cc(C#N)cnn12 ; CC(C)(O)c1cc2nn([C@H]3CC[C@H](N4CCN(C(=O)[C@@H]5CCN(c6ccc7c(c6)CN(C6CCC(=O)NC6=O)C7=O)C5)CC4)CC3)cc2cc1NC(=O)c1ccc2cc(C#N)cnn12]

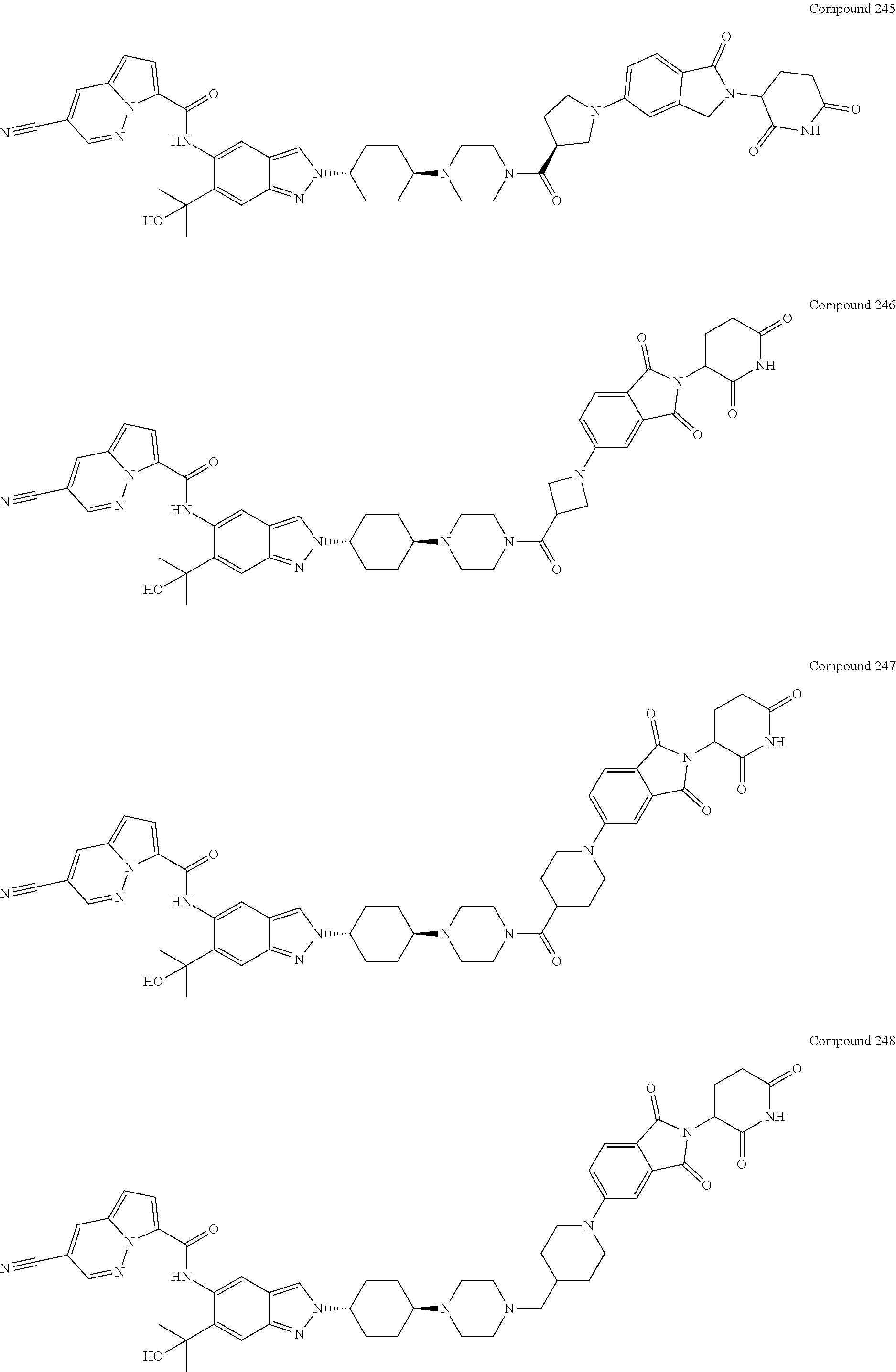

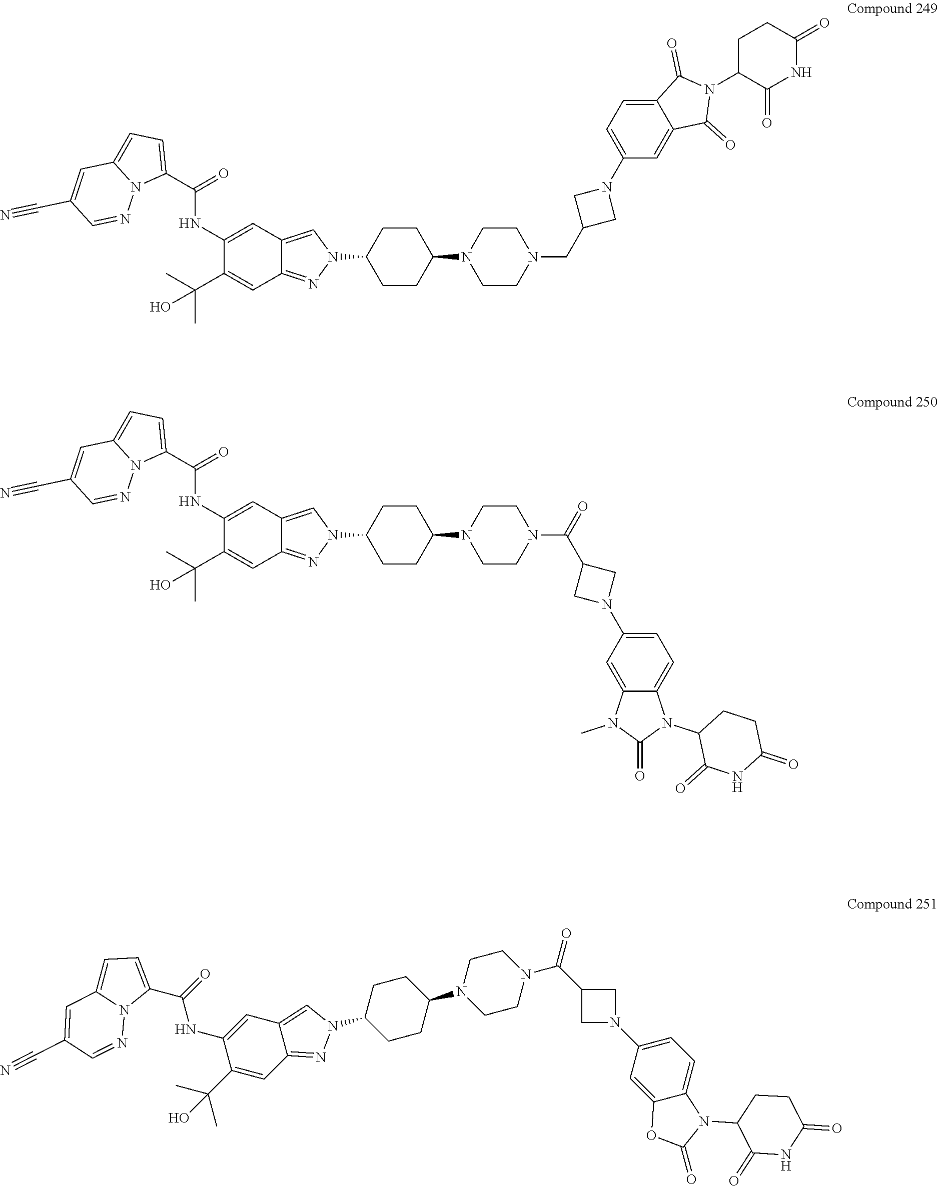 [SMILES: CC(C)(O)c1cc2nn([C@H]3CC[C@H](N4CCN(CC5CN(c6ccc7c(c6)C(=O)N(C6CCC(=O)NC6=O)C7=O)C5)CC4)CC3)cc2cc1NC(=O)c1ccc2cc(C#N)cnn12 ; Cn1c(=O)n(C2CCC(=O)NC2=O)c2ccc(N3CC(C(=O)N4CCN([C@H]5CC[C@H](n6cc7cc(NC(=O)c8ccc9cc(C#N)cnn89)c(C(C)(C)O)cc7n6)CC5)CC4)C3)cc21 ; CC(C)(O)c1cc2nn([C@H]3CC[C@H](N4CCN(C(=O)C5CN(c6ccc7c(c6)oc(=O)n7C6CCC(=O)NC6=O)C5)CC4)CC3)cc2cc1NC(=O)c1ccc2cc(C#N)cnn12]

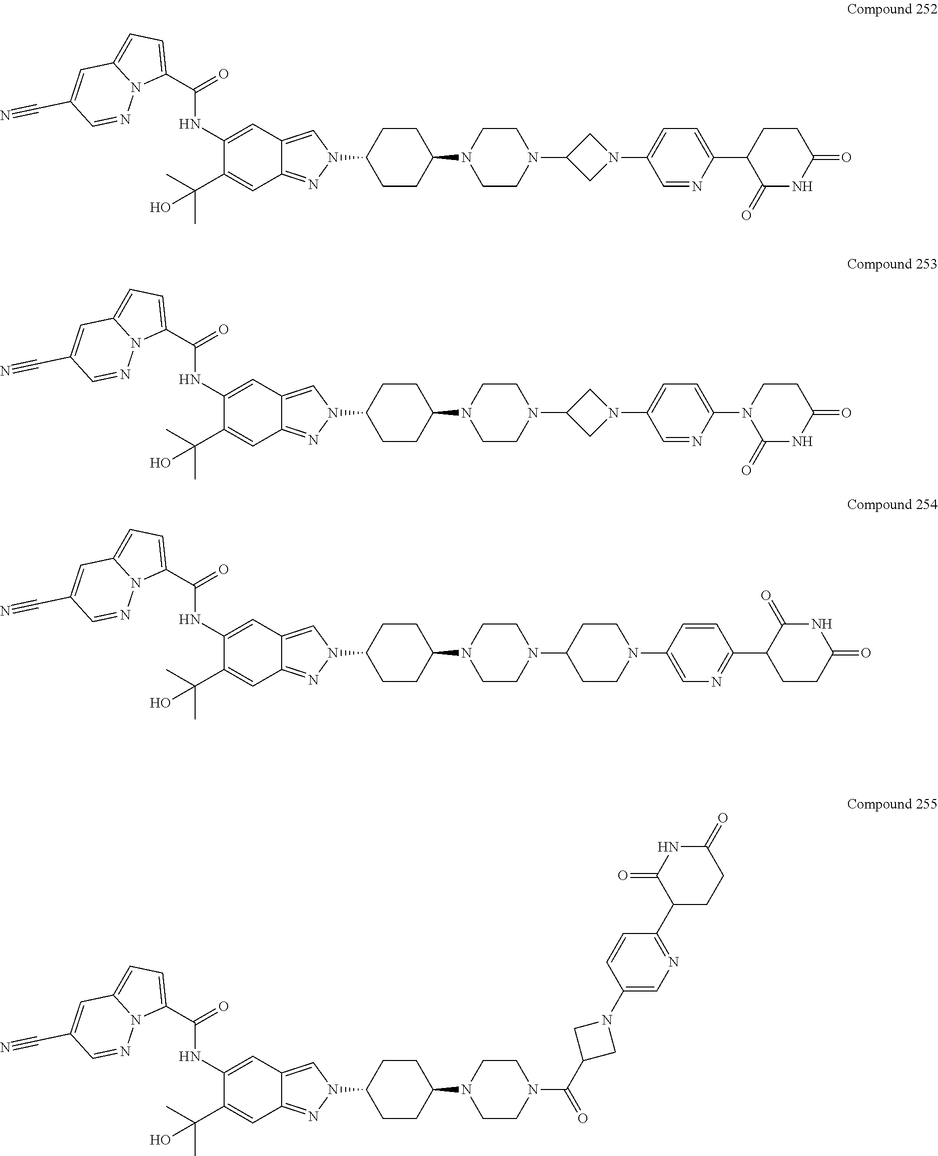 [SMILES: CC(C)(O)c1cc2nn([C@H]3CC[C@H](N4CCN(C5CN(c6ccc(C7CCC(=O)NC7=O)nc6)C5)CC4)CC3)cc2cc1NC(=O)c1ccc2cc(C#N)cnn12 ; CC(C)(O)c1cc2nn([C@H]3CC[C@H](N4CCN(C5CN(c6ccc(N7CCC(=O)NC7=O)nc6)C5)CC4)CC3)cc2cc1NC(=O)c1ccc2cc(C#N)cnn12 ; CC(C)(O)c1cc2nn([C@H]3CC[C@H](N4CCN(C5CCN(c6ccc(C7CCC(=O)NC7=O)nc6)CC5)CC4)CC3)cc2cc1NC(=O)c1ccc2cc(C#N)cnn12 ; CC(C)(O)c1cc2nn([C@H]3CC[C@H](N4CCN(C(=O)C5CN(c6ccc(C7CCC(=O)NC7=O)nc6)C5)CC4)CC3)cc2cc1NC(=O)c1ccc2cc(C#N)cnn12]

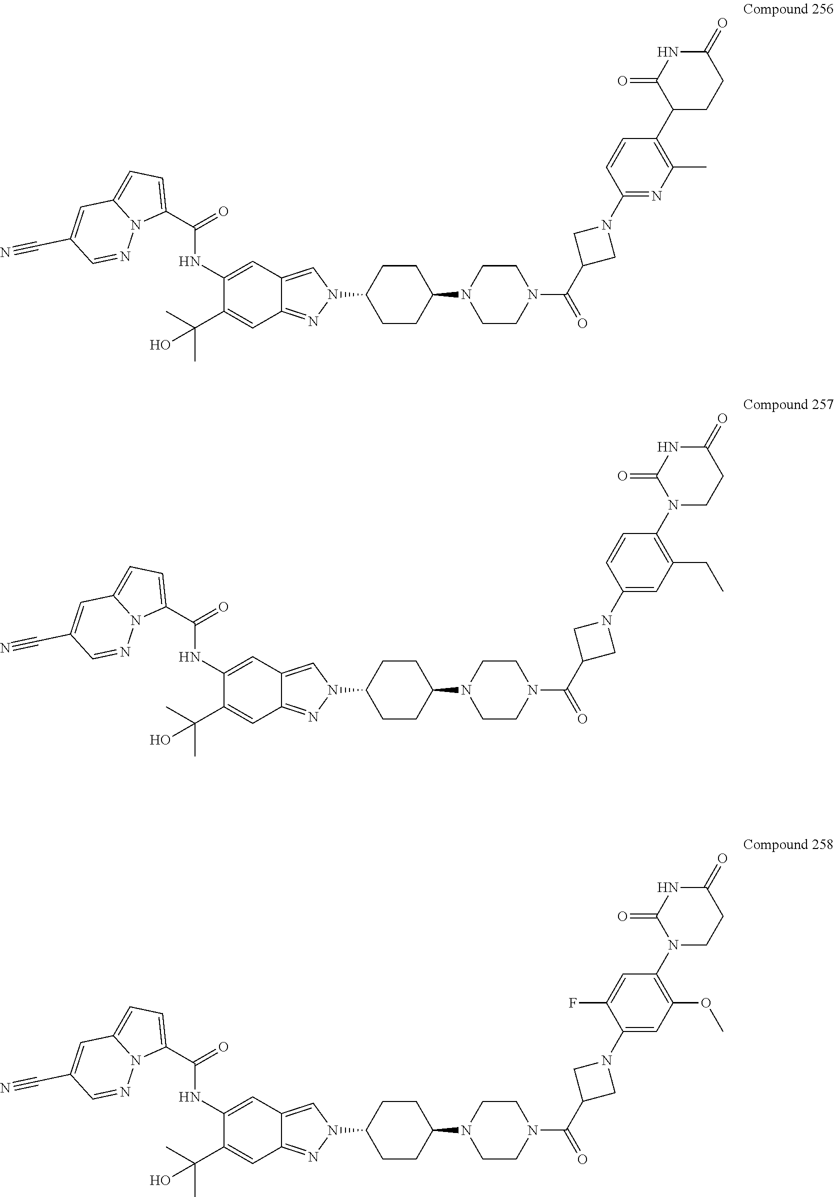 [SMILES: Cc1nc(N2CC(C(=O)N3CCN([C@H]4CC[C@H](n5cc6cc(NC(=O)c7ccc8cc(C#N)cnn78)c(C(C)(C)O)cc6n5)CC4)CC3)C2)ccc1C1CCC(=O)NC1=O ; CCc1cc(N2CC(C(=O)N3CCN([C@H]4CC[C@H](n5cc6cc(NC(=O)c7ccc8cc(C#N)cnn78)c(C(C)(C)O)cc6n5)CC4)CC3)C2)ccc1N1CCC(=O)NC1=O ; COc1cc(N2CC(C(=O)N3CCN([C@H]4CC[C@H](n5cc6cc(NC(=O)c7ccc8cc(C#N)cnn78)c(C(C)(C)O)cc6n5)CC4)CC3)C2)c(F)cc1N1CCC(=O)NC1=O]

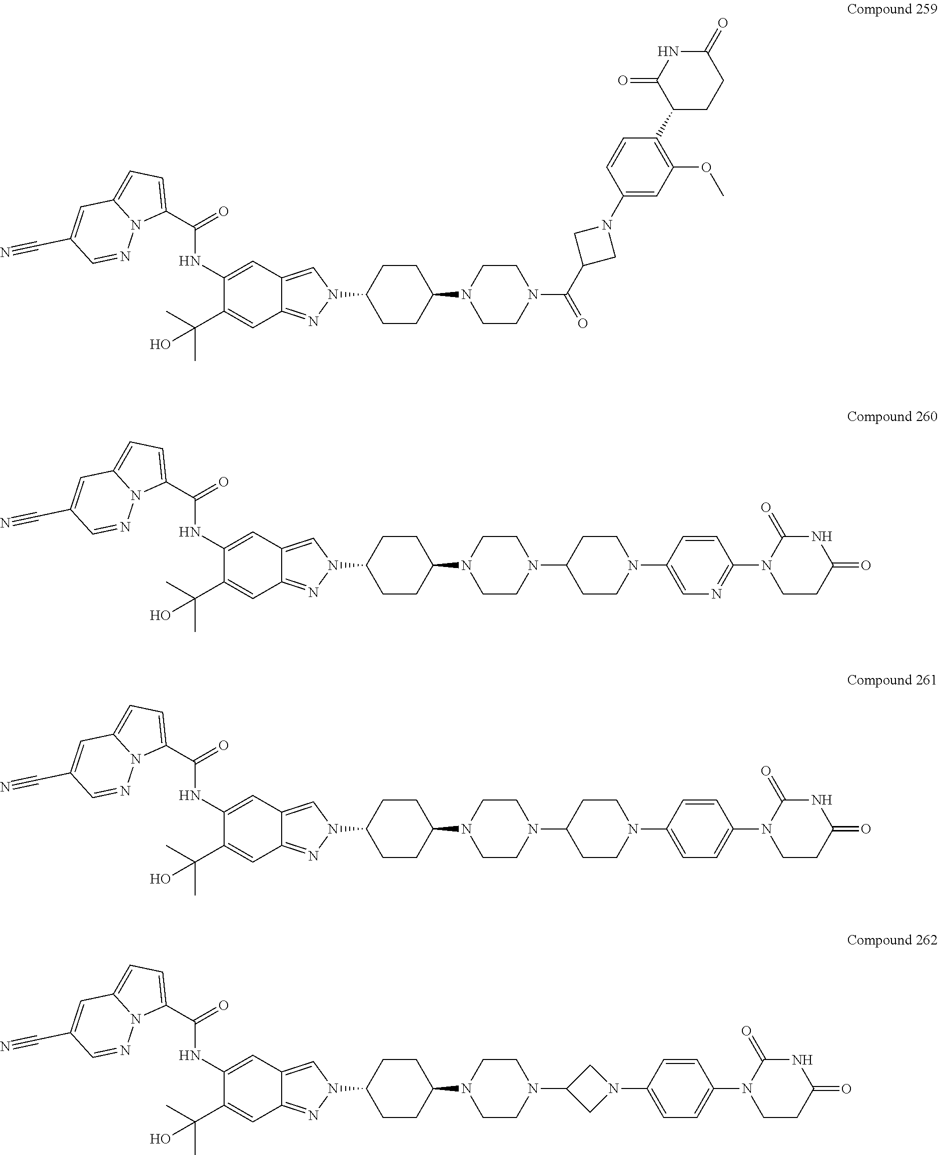 [SMILES: COc1cc(N2CC(C(=O)N3CCN([C@H]4CC[C@H](n5cc6cc(NC(=O)c7ccc8cc(C#N)cnn78)c(C(C)(C)O)cc6n5)CC4)CC3)C2)ccc1[C@H]1CCC(=O)NC1=O ; CC(C)(O)c1cc2nn([C@H]3CC[C@H](N4CCN(C5CCN(c6ccc(N7CCC(=O)NC7=O)nc6)CC5)CC4)CC3)cc2cc1NC(=O)c1ccc2cc(C#N)cnn12 ; CC(C)(O)c1cc2nn([C@H]3CC[C@H](N4CCN(C5CCN(c6ccc(N7CCC(=O)NC7=O)cc6)CC5)CC4)CC3)cc2cc1NC(=O)c1ccc2cc(C#N)cnn12 ; CC(C)(O)c1cc2nn([C@H]3CC[C@H](N4CCN(C5CN(c6ccc(N7CCC(=O)NC7=O)cc6)C5)CC4)CC3)cc2cc1NC(=O)c1ccc2cc(C#N)cnn12]

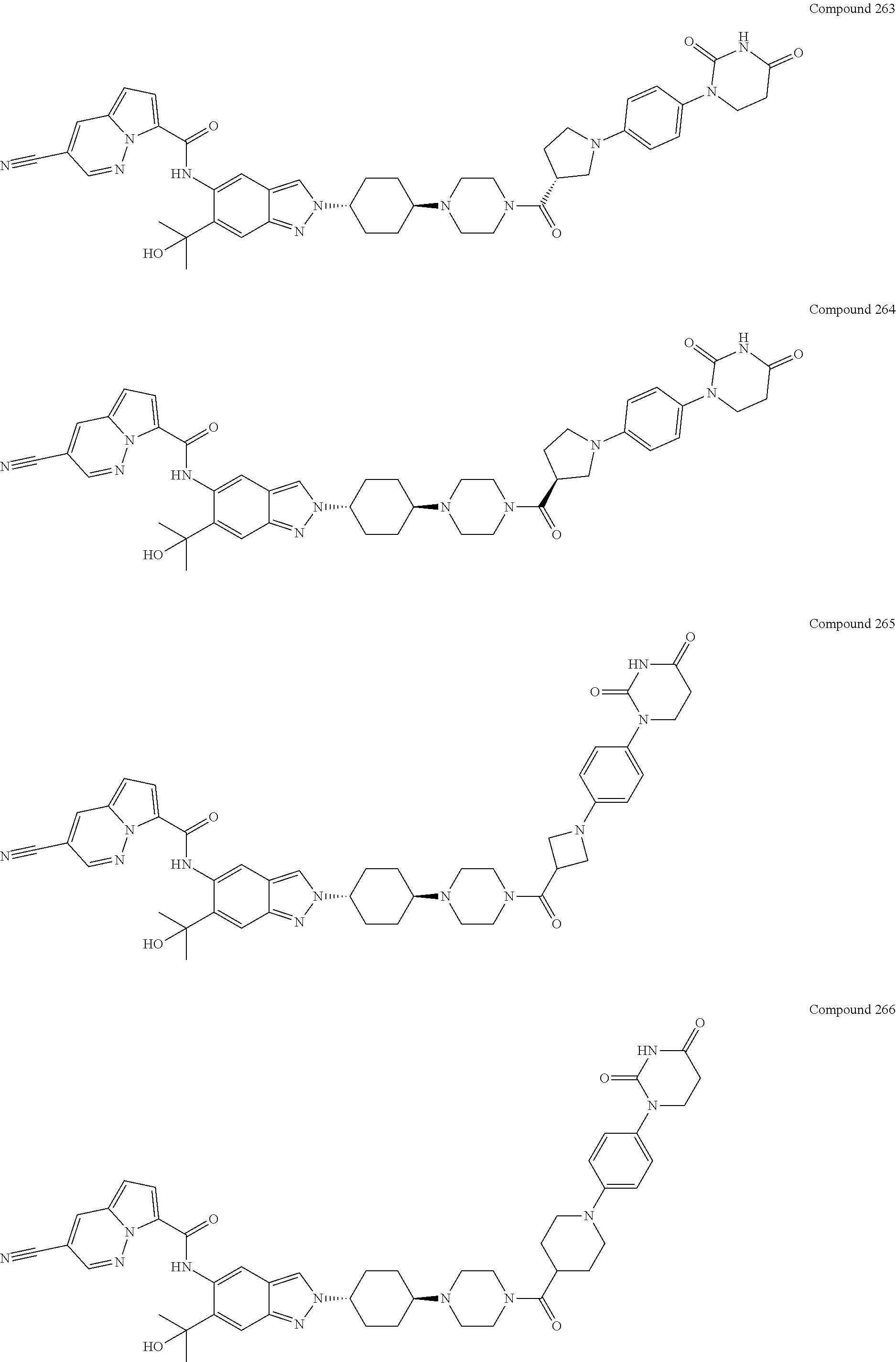 [SMILES: CC(C)(O)c1cc2nn([C@H]3CC[C@H](N4CCN(C(=O)[C@@H]5CCN(c6ccc(N7CCC(=O)NC7=O)cc6)C5)CC4)CC3)cc2cc1NC(=O)c1ccc2cc(C#N)cnn12 ; CC(C)(O)c1cc2nn([C@H]3CC[C@H](N4CCN(C(=O)[C@H]5CCN(c6ccc(N7CCC(=O)NC7=O)cc6)C5)CC4)CC3)cc2cc1NC(=O)c1ccc2cc(C#N)cnn12 ; CC(C)(O)c1cc2nn([C@H]3CC[C@H](N4CCN(C(=O)C5CN(c6ccc(N7CCC(=O)NC7=O)cc6)C5)CC4)CC3)cc2cc1NC(=O)c1ccc2cc(C#N)cnn12 ; CC(C)(O)c1cc2nn([C@H]3CC[C@H](N4CCN(C(=O)C5CCN(c6ccc(N7CCC(=O)NC7=O)cc6)CC5)CC4)CC3)cc2cc1NC(=O)c1ccc2cc(C#N)cnn12]

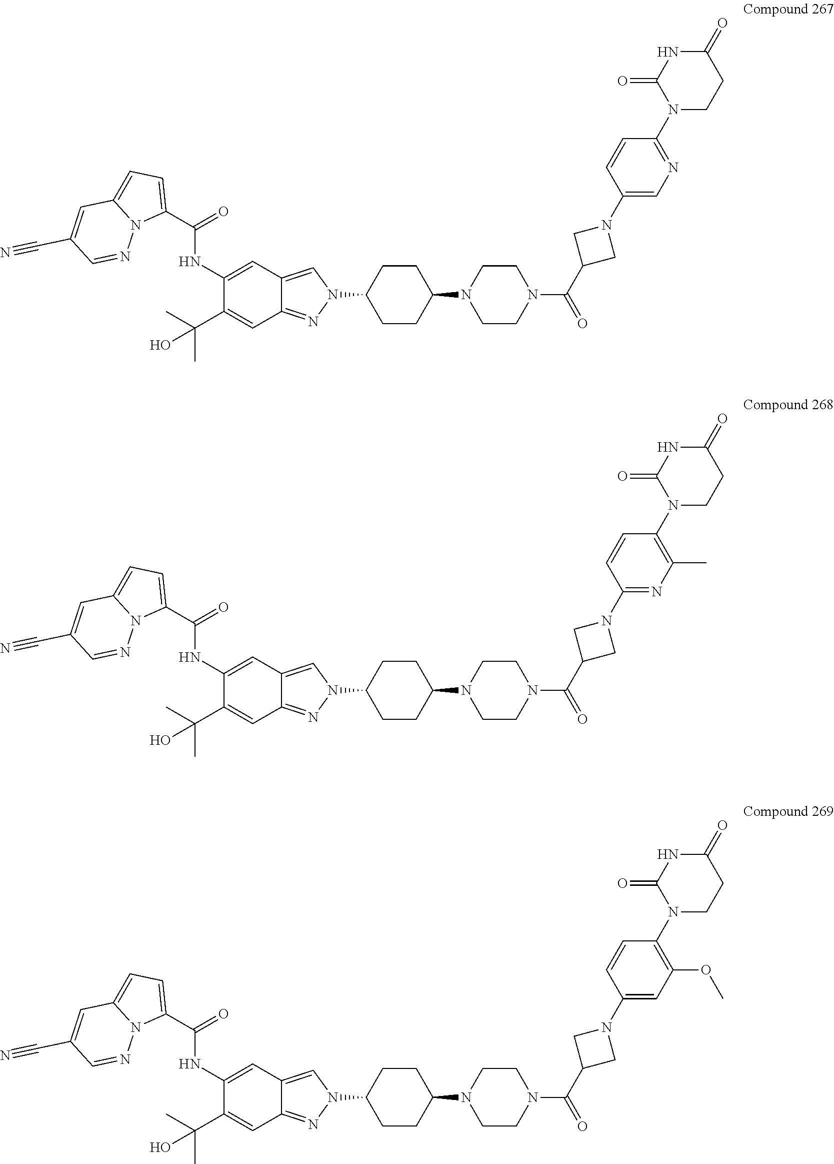 [SMILES: CC(C)(O)c1cc2nn([C@H]3CC[C@H](N4CCN(C(=O)C5CN(c6ccc(N7CCC(=O)NC7=O)nc6)C5)CC4)CC3)cc2cc1NC(=O)c1ccc2cc(C#N)cnn12 ; Cc1nc(N2CC(C(=O)N3CCN([C@H]4CC[C@H](n5cc6cc(NC(=O)c7ccc8cc(C#N)cnn78)c(C(C)(C)O)cc6n5)CC4)CC3)C2)ccc1N1CCC(=O)NC1=O ; COc1cc(N2CC(C(=O)N3CCN([C@H]4CC[C@H](n5cc6cc(NC(=O)c7ccc8cc(C#N)cnn78)c(C(C)(C)O)cc6n5)CC4)CC3)C2)ccc1N1CCC(=O)NC1=O]

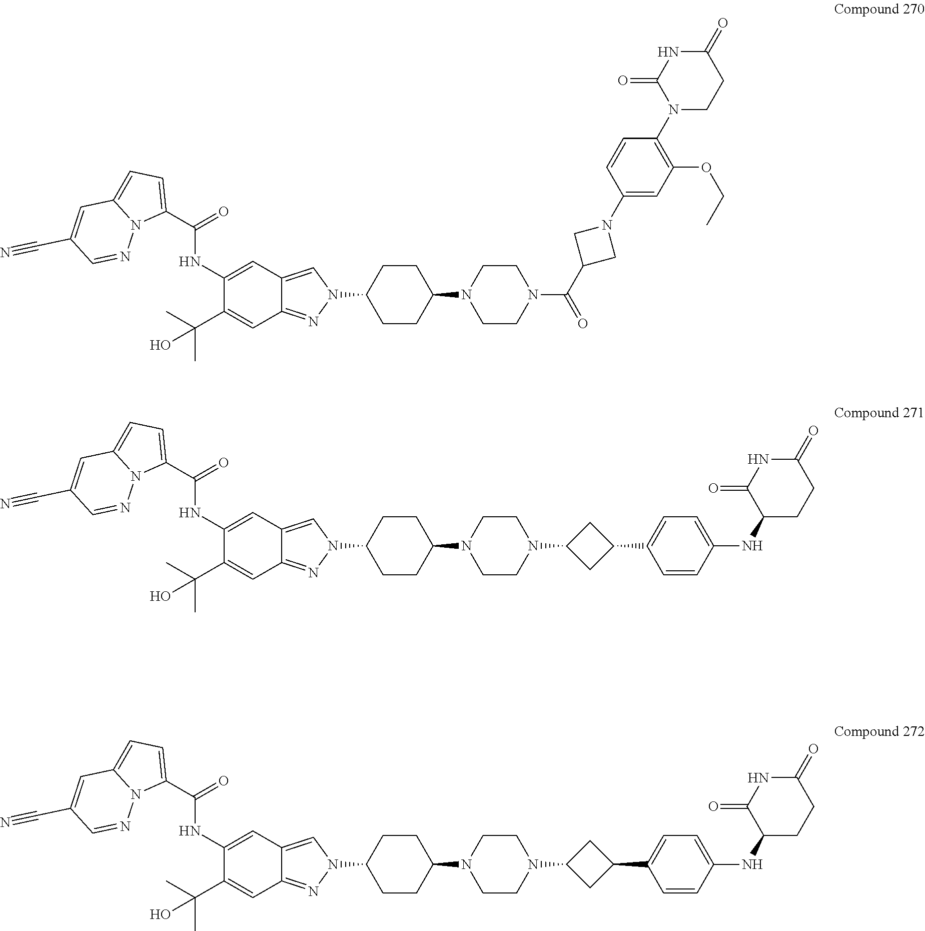 [SMILES: CCOc1cc(N2CC(C(=O)N3CCN([C@H]4CC[C@H](n5cc6cc(NC(=O)c7ccc8cc(C#N)cnn78)c(C(C)(C)O)cc6n5)CC4)CC3)C2)ccc1N1CCC(=O)NC1=O ; CC(C)(O)c1cc2nn([C@H]3CC[C@H](N4CCN([C@H]5C[C@@H](c6ccc(N[C@@H]7CCC(=O)NC7=O)cc6)C5)CC4)CC3)cc2cc1NC(=O)c1ccc2cc(C#N)cnn12 ; CC(C)(O)c1cc2nn([C@H]3CC[C@H](N4CCN([C@H]5C[C@H](c6ccc(N[C@@H]7CCC(=O)NC7=O)cc6)C5)CC4)CC3)cc2cc1NC(=O)c1ccc2cc(C#N)cnn12]

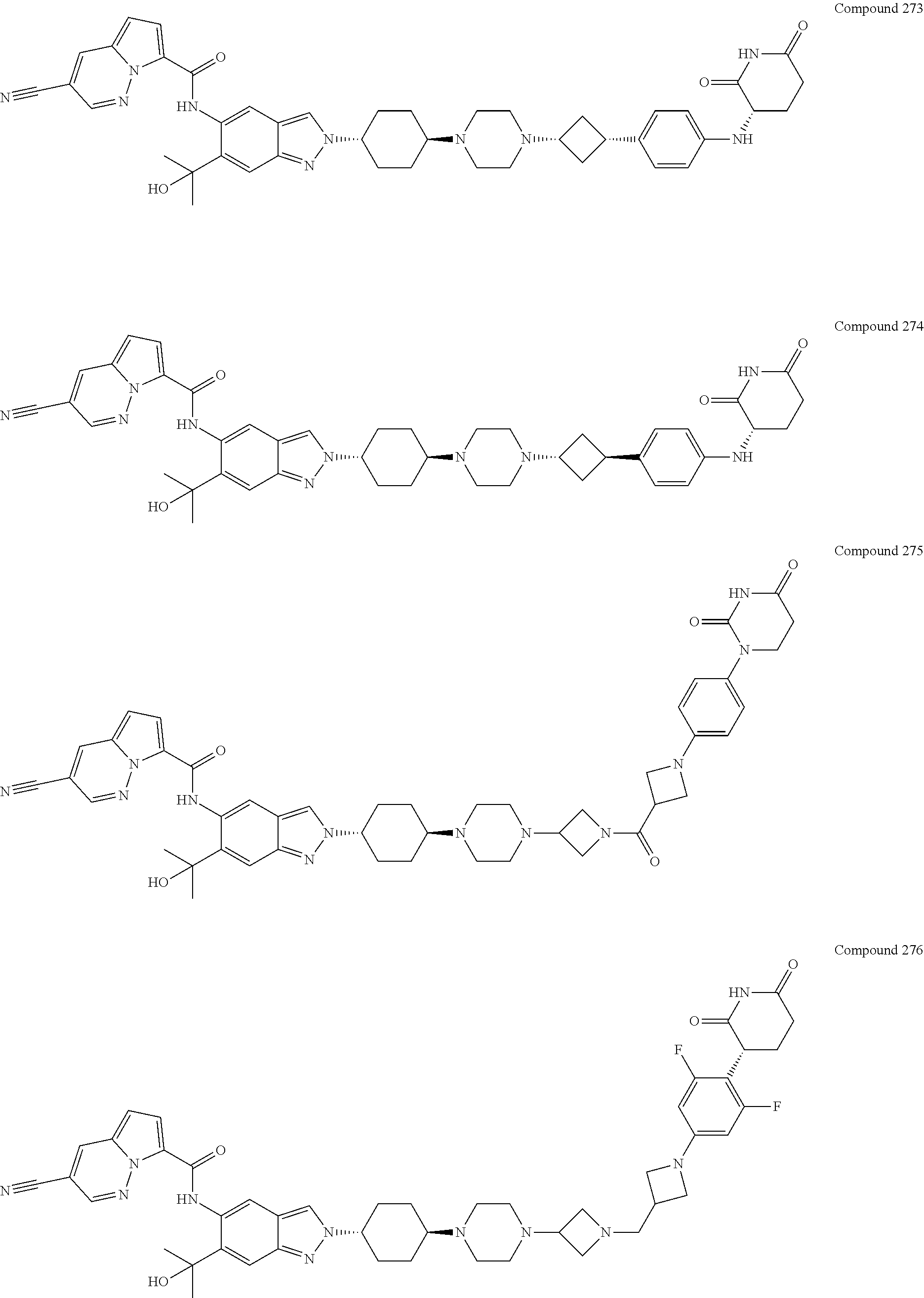 [SMILES: CC(C)(O)c1cc2nn([C@H]3CC[C@H](N4CCN([C@H]5C[C@@H](c6ccc(N[C@H]7CCC(=O)NC7=O)cc6)C5)CC4)CC3)cc2cc1NC(=O)c1ccc2cc(C#N)cnn12 ; CC(C)(O)c1cc2nn([C@H]3CC[C@H](N4CCN([C@H]5C[C@H](c6ccc(N[C@H]7CCC(=O)NC7=O)cc6)C5)CC4)CC3)cc2cc1NC(=O)c1ccc2cc(C#N)cnn12 ; CC(C)(O)c1cc2nn([C@H]3CC[C@H](N4CCN(C5CN(C(=O)C6CN(c7ccc(N8CCC(=O)NC8=O)cc7)C6)C5)CC4)CC3)cc2cc1NC(=O)c1ccc2cc(C#N)cnn12 ; CC(C)(O)c1cc2nn([C@H]3CC[C@H](N4CCN(C5CN(CC6CN(c7cc(F)c([C@H]8CCC(=O)NC8=O)c(F)c7)C6)C5)CC4)CC3)cc2cc1NC(=O)c1ccc2cc(C#N)cnn12]

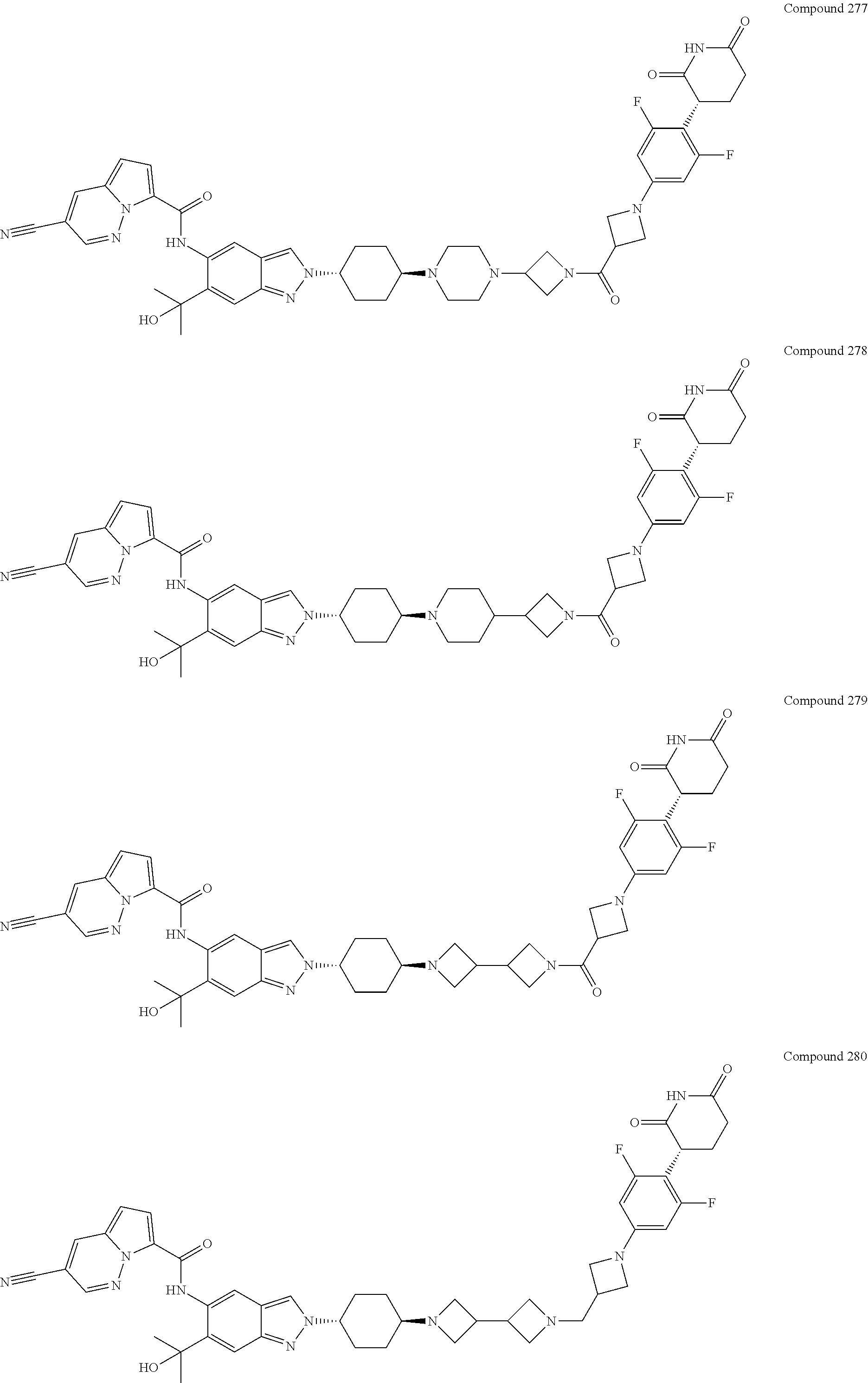

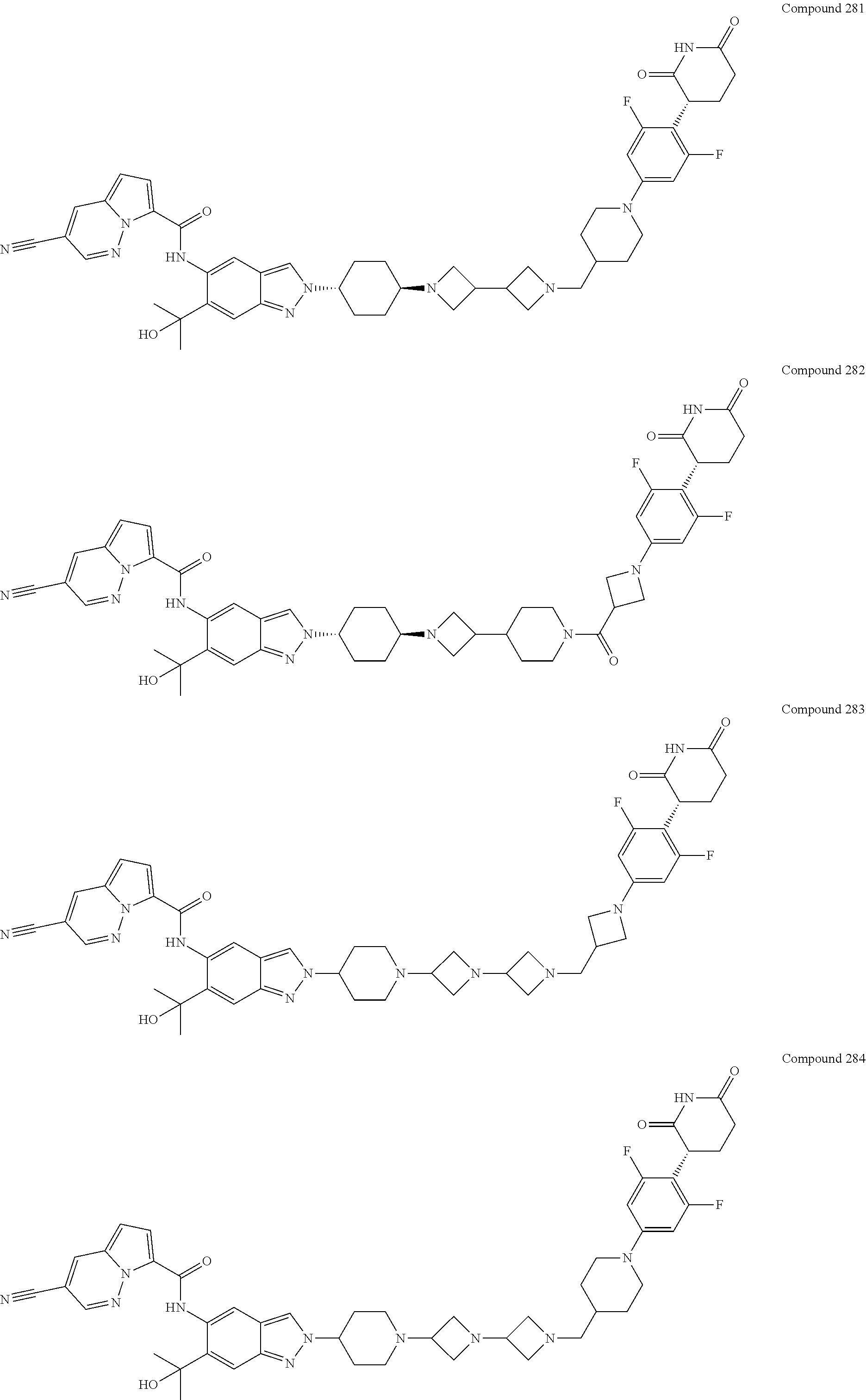 [SMILES: CC(C)(O)c1cc2nn([C@H]3CC[C@H](N4CC(C5CN(CC6CCN(c7cc(F)c([C@H]8CCC(=O)NC8=O)c(F)c7)CC6)C5)C4)CC3)cc2cc1NC(=O)c1ccc2cc(C#N)cnn12 ; CC(C)(O)c1cc2nn([C@H]3CC[C@H](N4CC(C5CCN(C(=O)C6CN(c7cc(F)c([C@H]8CCC(=O)NC8=O)c(F)c7)C6)CC5)C4)CC3)cc2cc1NC(=O)c1ccc2cc(C#N)cnn12 ; CC(C)(O)c1cc2nn(C3CCN(C4CN(C5CN(CC6CN(c7cc(F)c([C@H]8CCC(=O)NC8=O)c(F)c7)C6)C5)C4)CC3)cc2cc1NC(=O)c1ccc2cc(C#N)cnn12 ; CC(C)(O)c1cc2nn(C3CCN(C4CN(C5CN(CC6CCN(c7cc(F)c([C@H]8CCC(=O)NC8=O)c(F)c7)CC6)C5)C4)CC3)cc2cc1NC(=O)c1ccc2cc(C#N)cnn12]

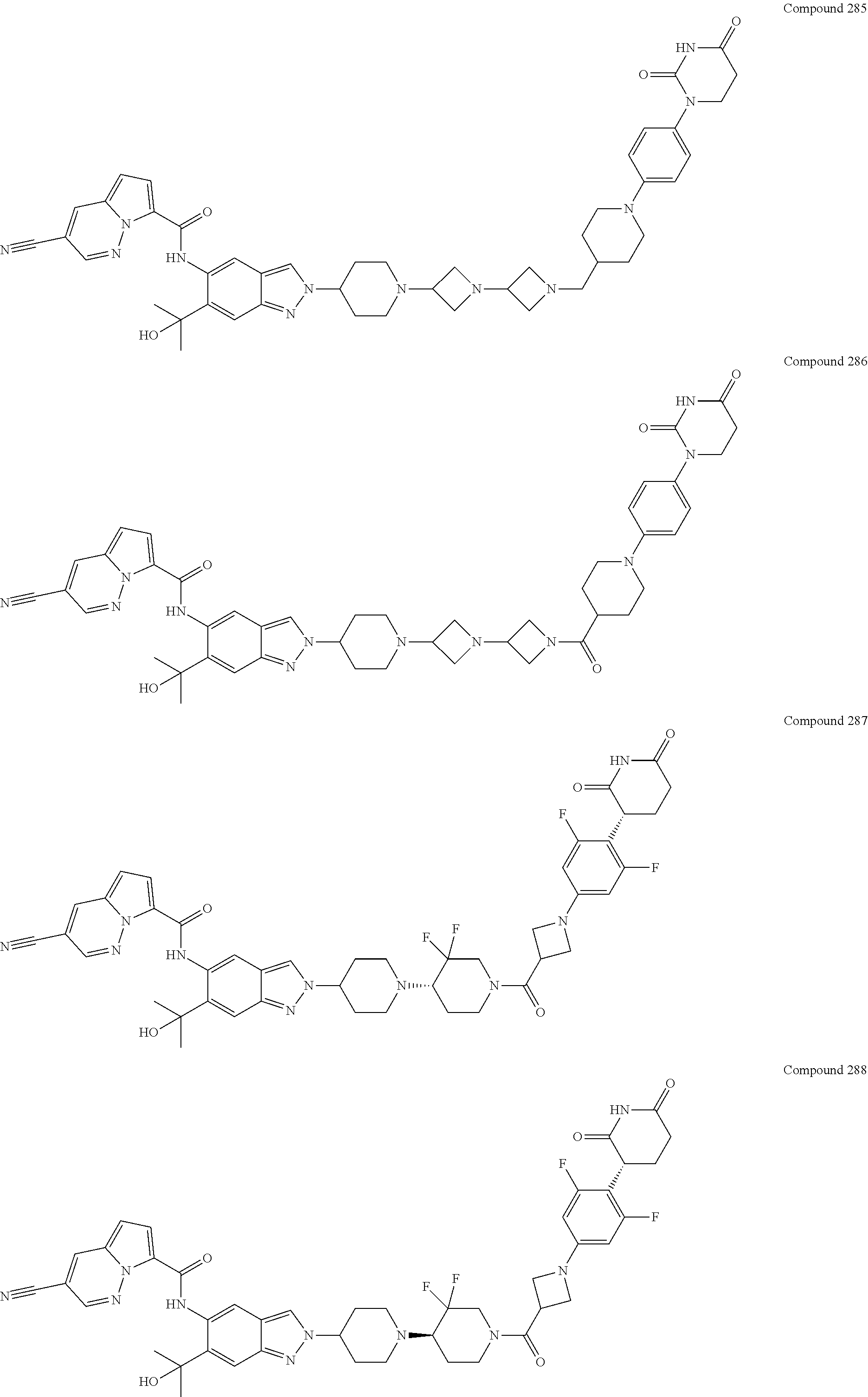 [SMILES: CC(C)(O)c1cc2nn(C3CCN(C4CN(C5CN(CC6CCN(c7ccc(N8CCC(=O)NC8=O)cc7)CC6)C5)C4)CC3)cc2cc1NC(=O)c1ccc2cc(C#N)cnn12 ; CC(C)(O)c1cc2nn(C3CCN(C4CN(C5CN(C(=O)C6CCN(c7ccc(N8CCC(=O)NC8=O)cc7)CC6)C5)C4)CC3)cc2cc1NC(=O)c1ccc2cc(C#N)cnn12 ; CC(C)(O)c1cc2nn(C3CCN([C@H]4CCN(C(=O)C5CN(c6cc(F)c([C@H]7CCC(=O)NC7=O)c(F)c6)C5)CC4(F)F)CC3)cc2cc1NC(=O)c1ccc2cc(C#N)cnn12 ; CC(C)(O)c1cc2nn(C3CCN([C@@H]4CCN(C(=O)C5CN(c6cc(F)c([C@H]7CCC(=O)NC7=O)c(F)c6)C5)CC4(F)F)CC3)cc2cc1NC(=O)c1ccc2cc(C#N)cnn12]

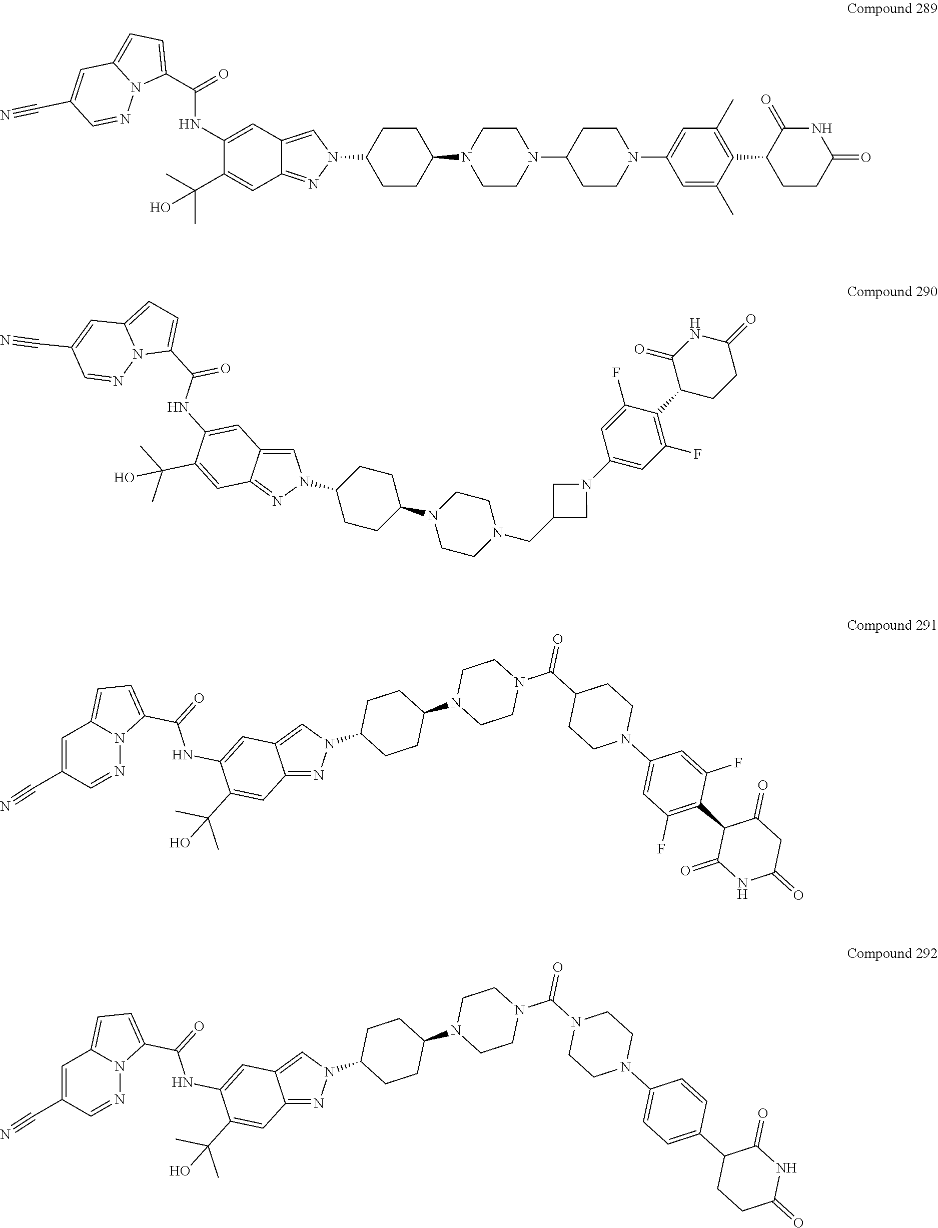

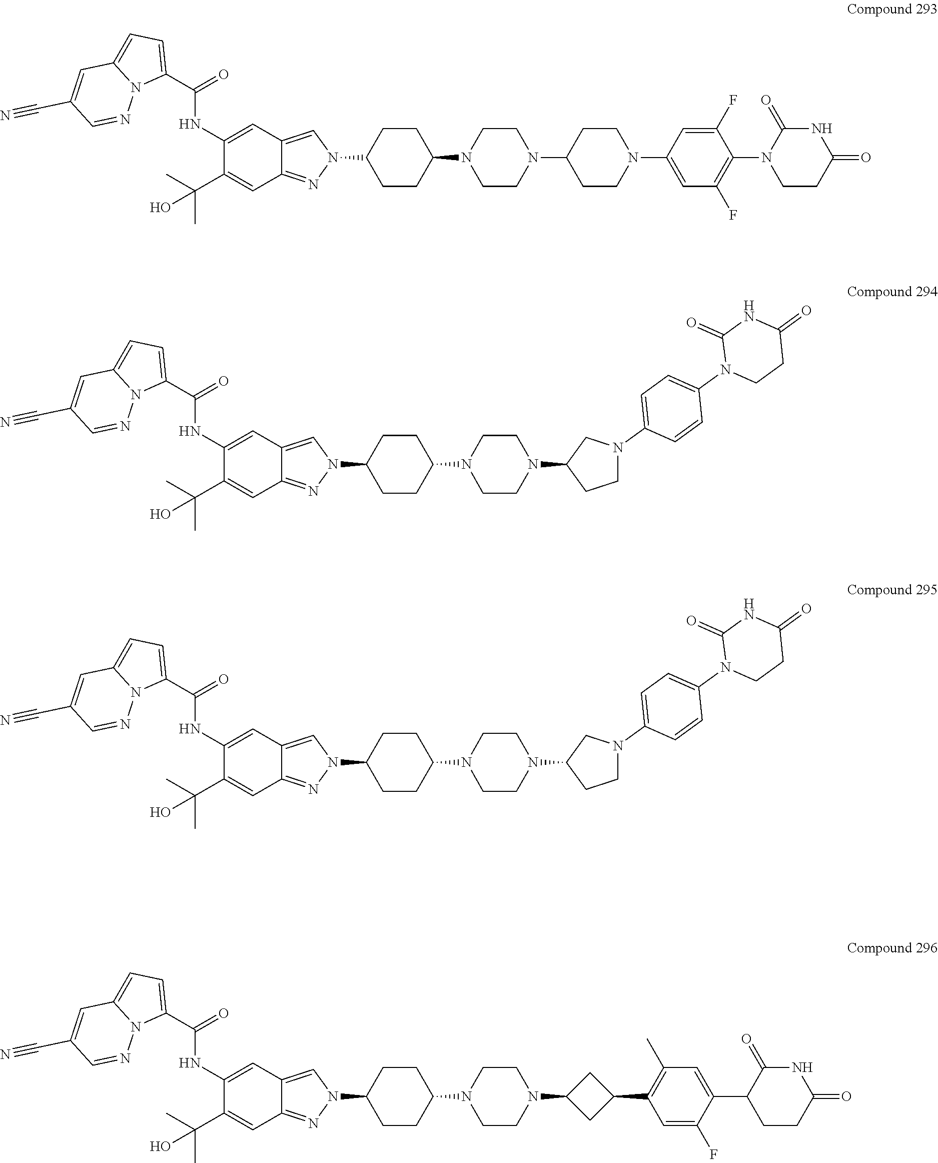

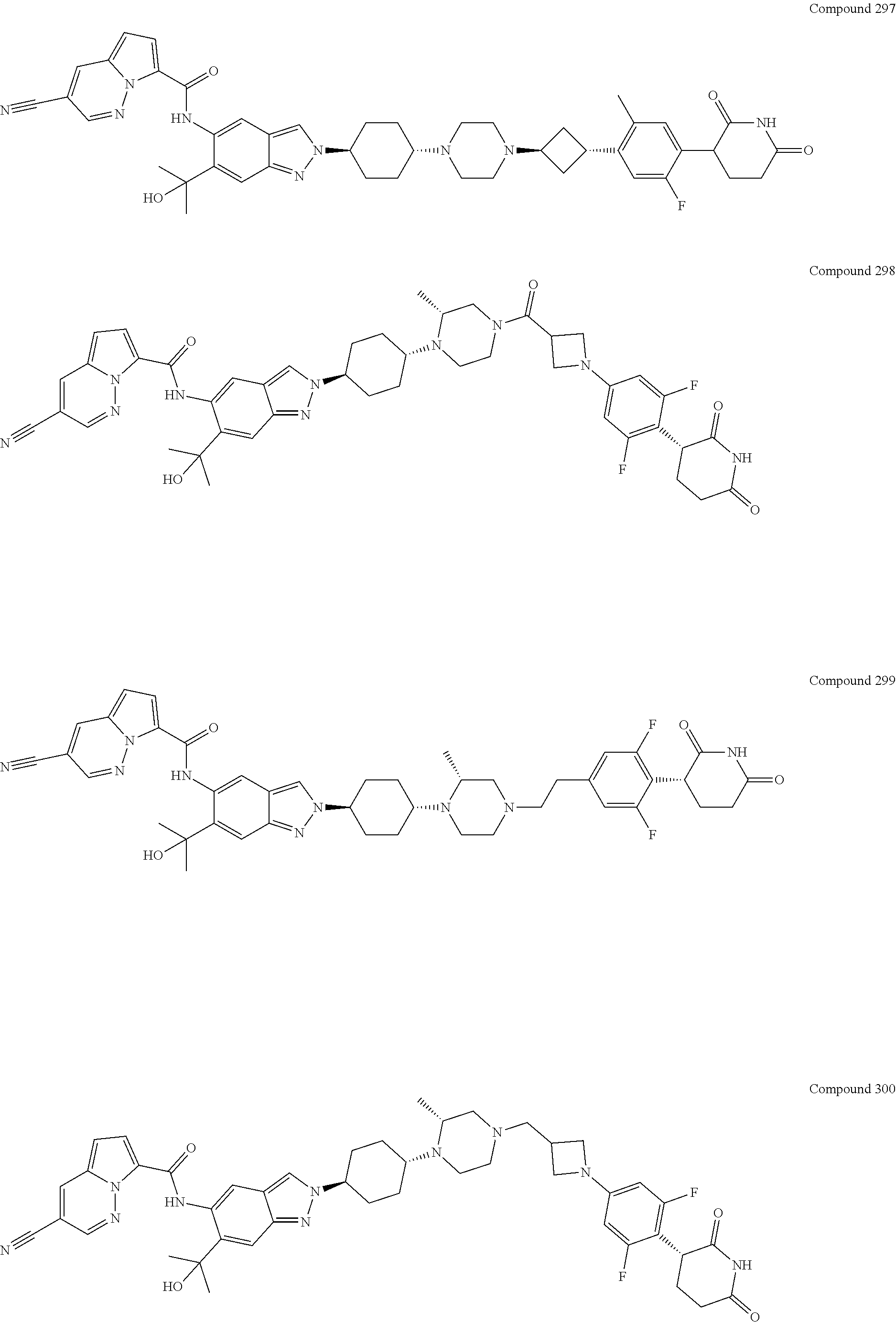

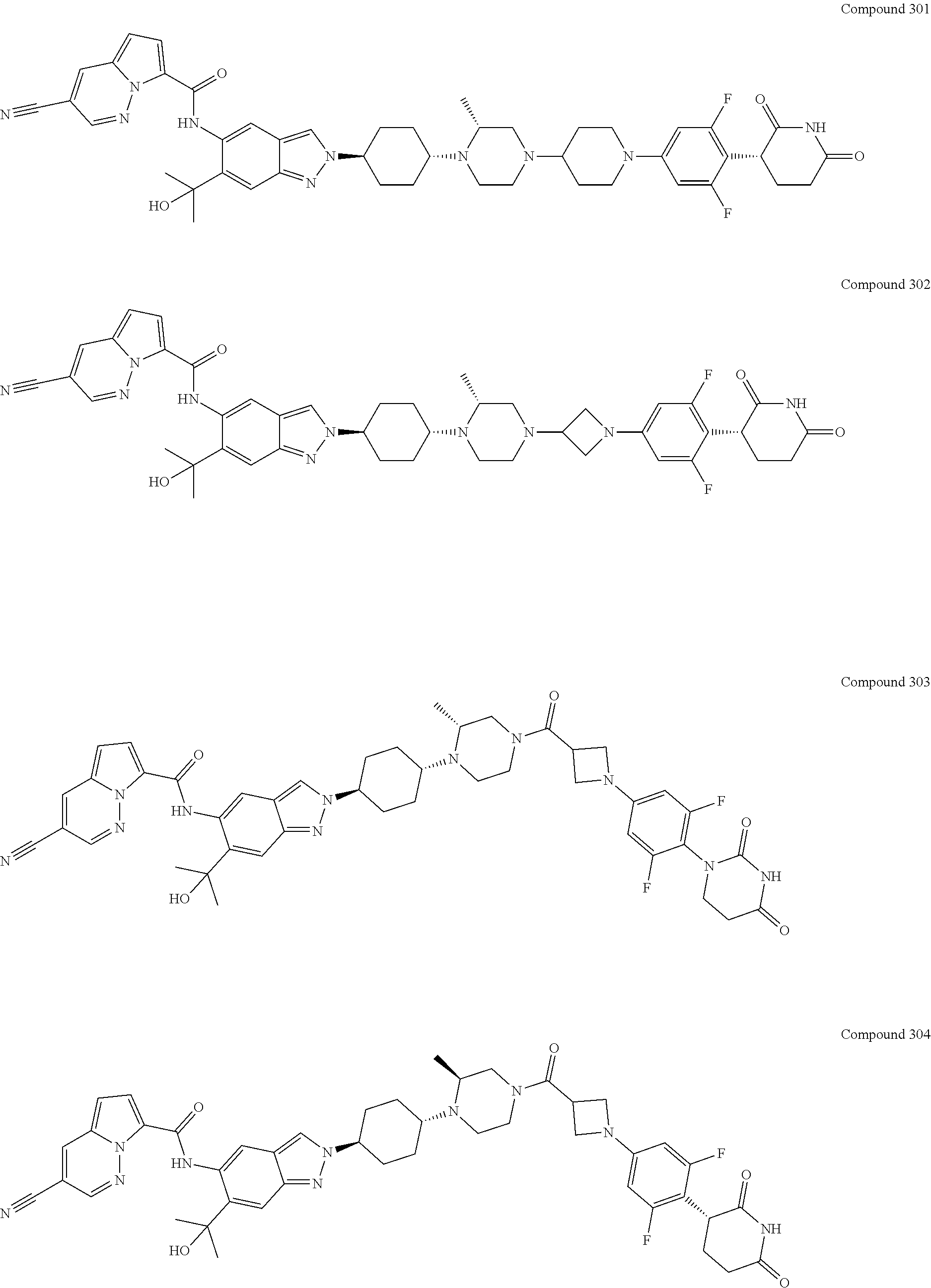

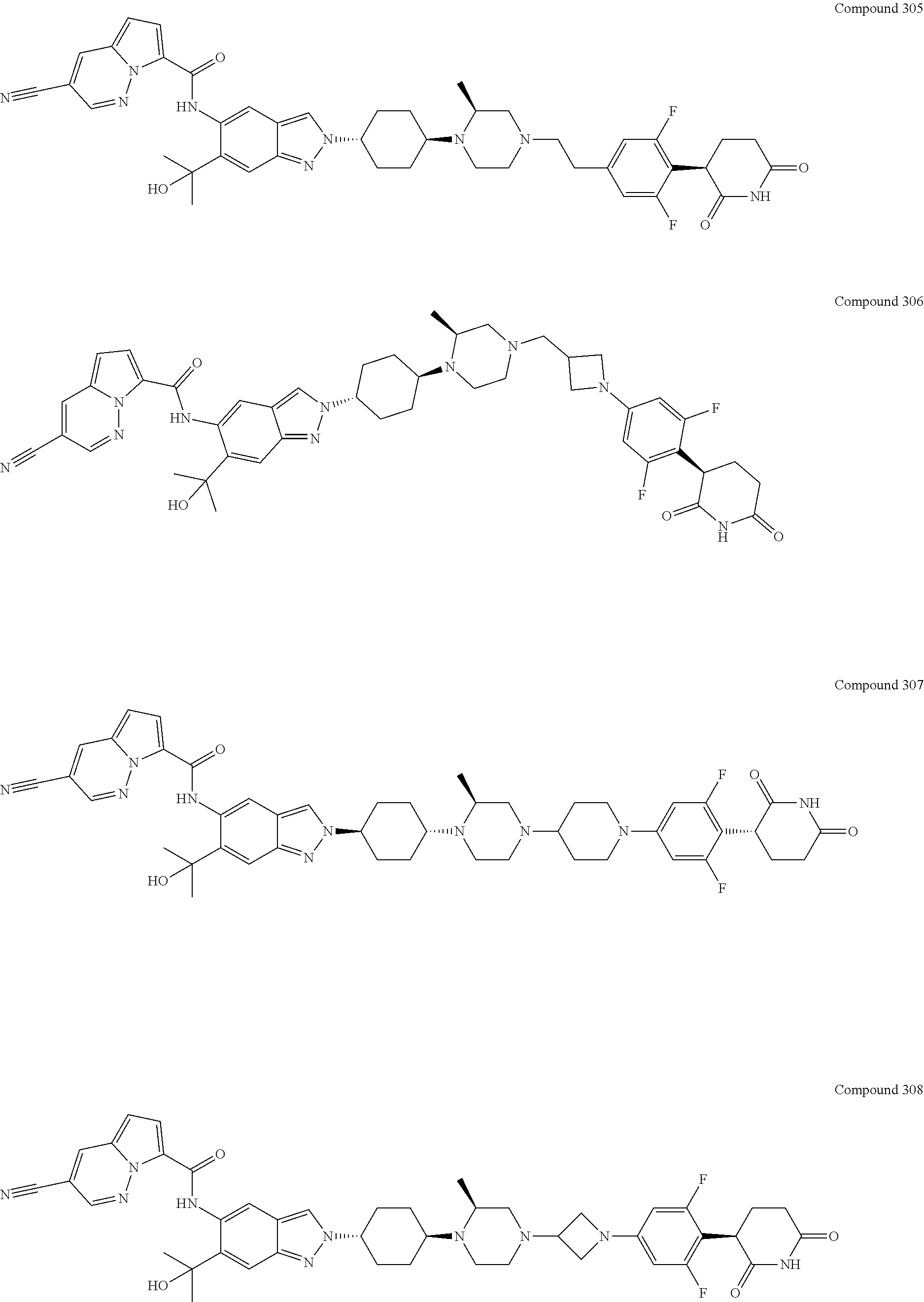

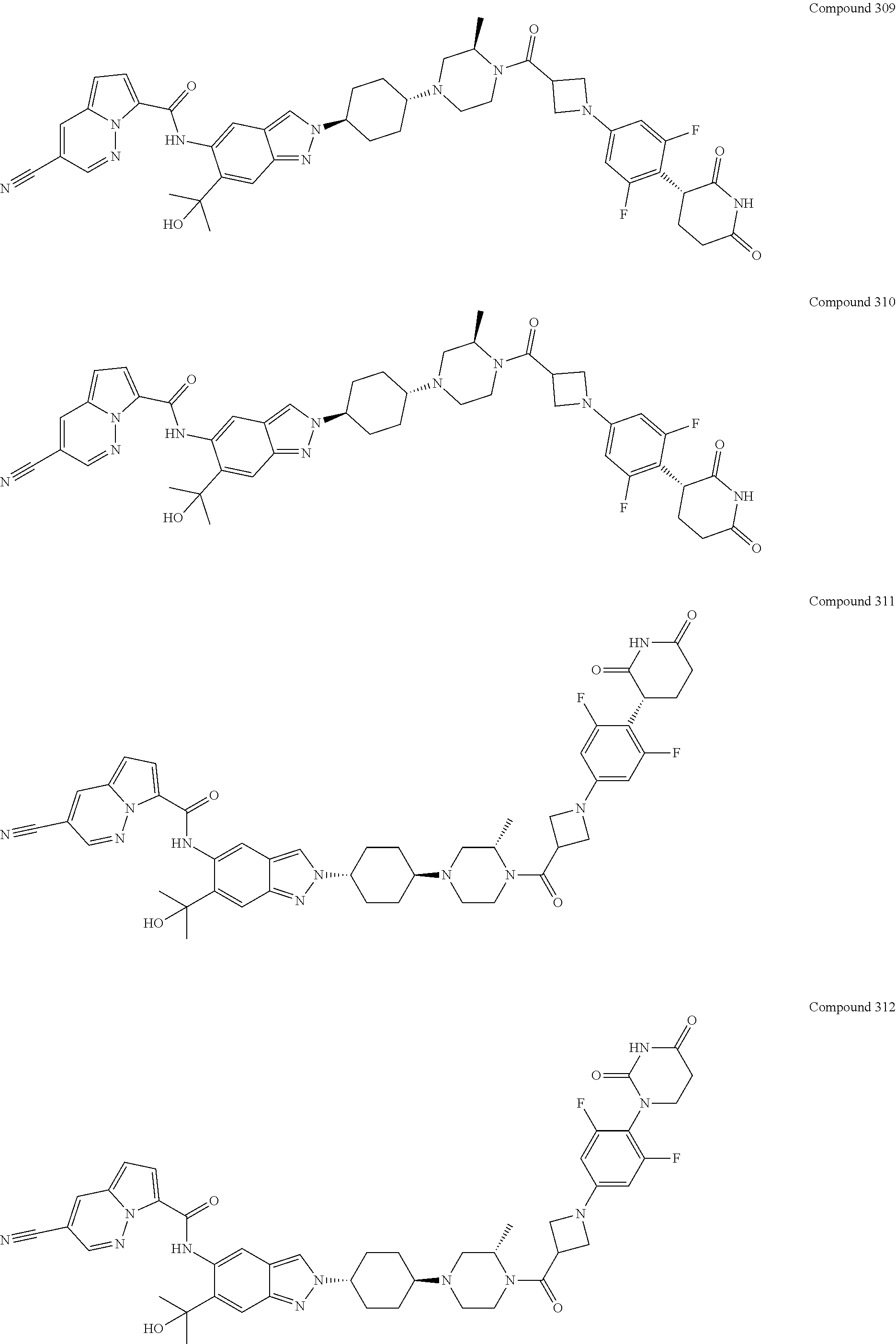

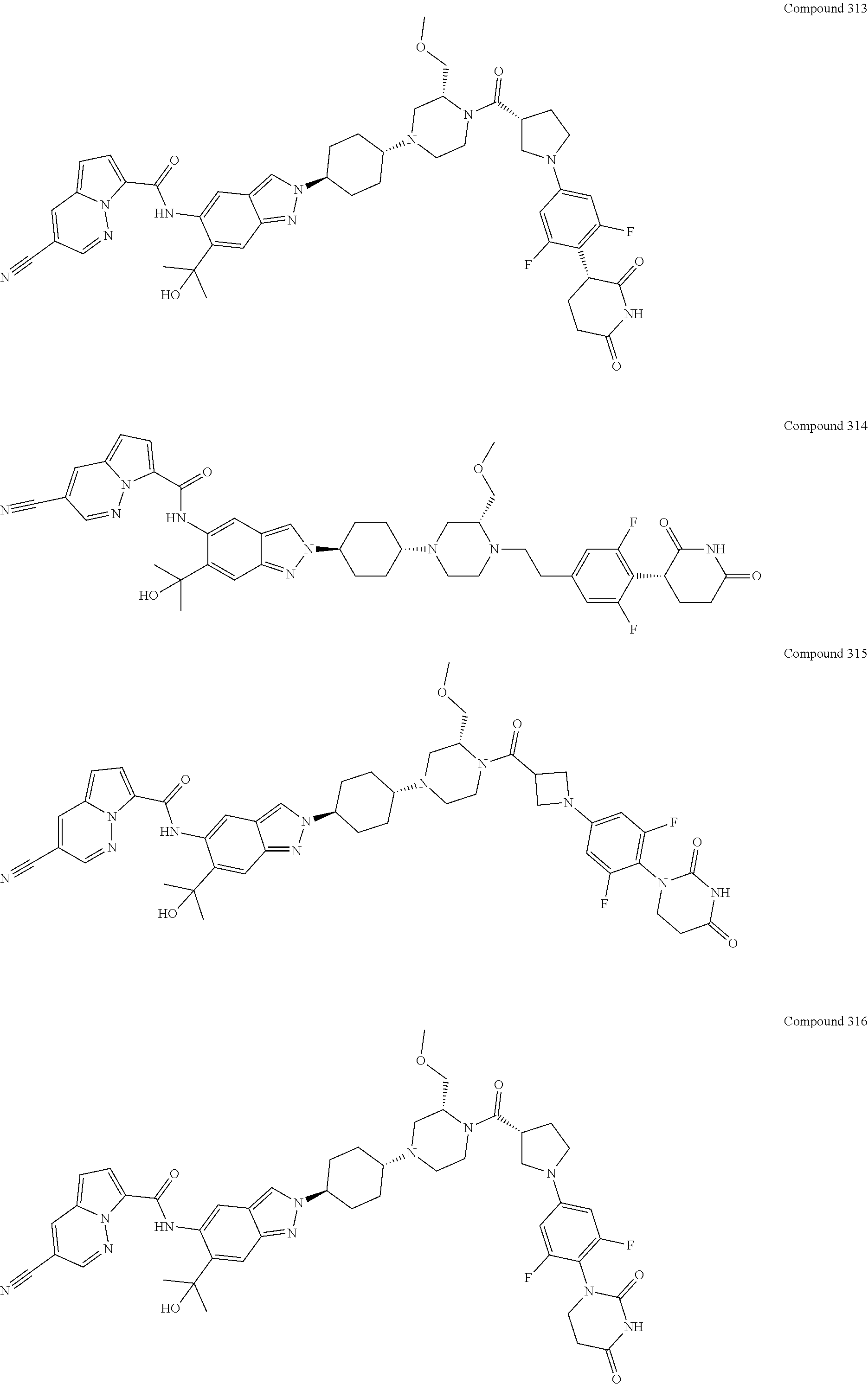

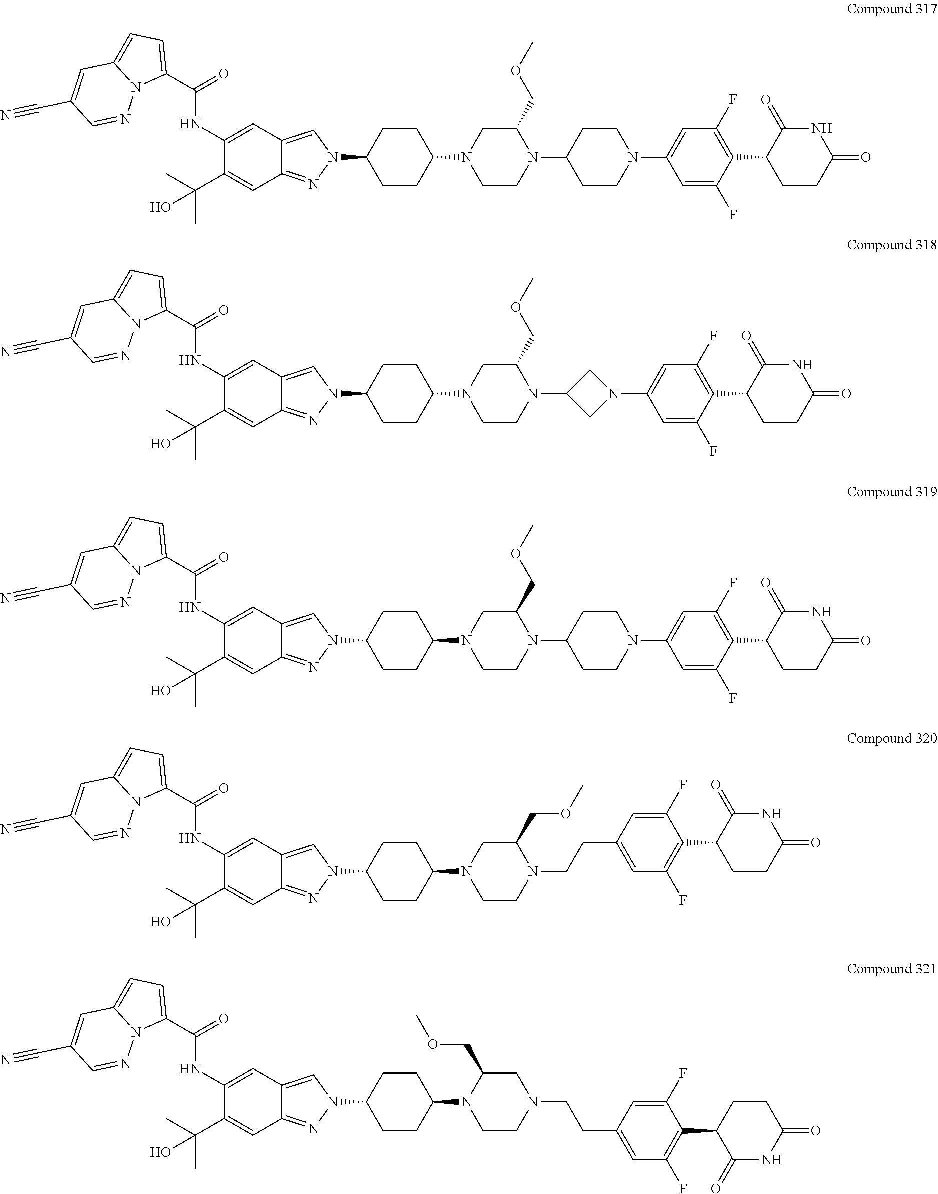

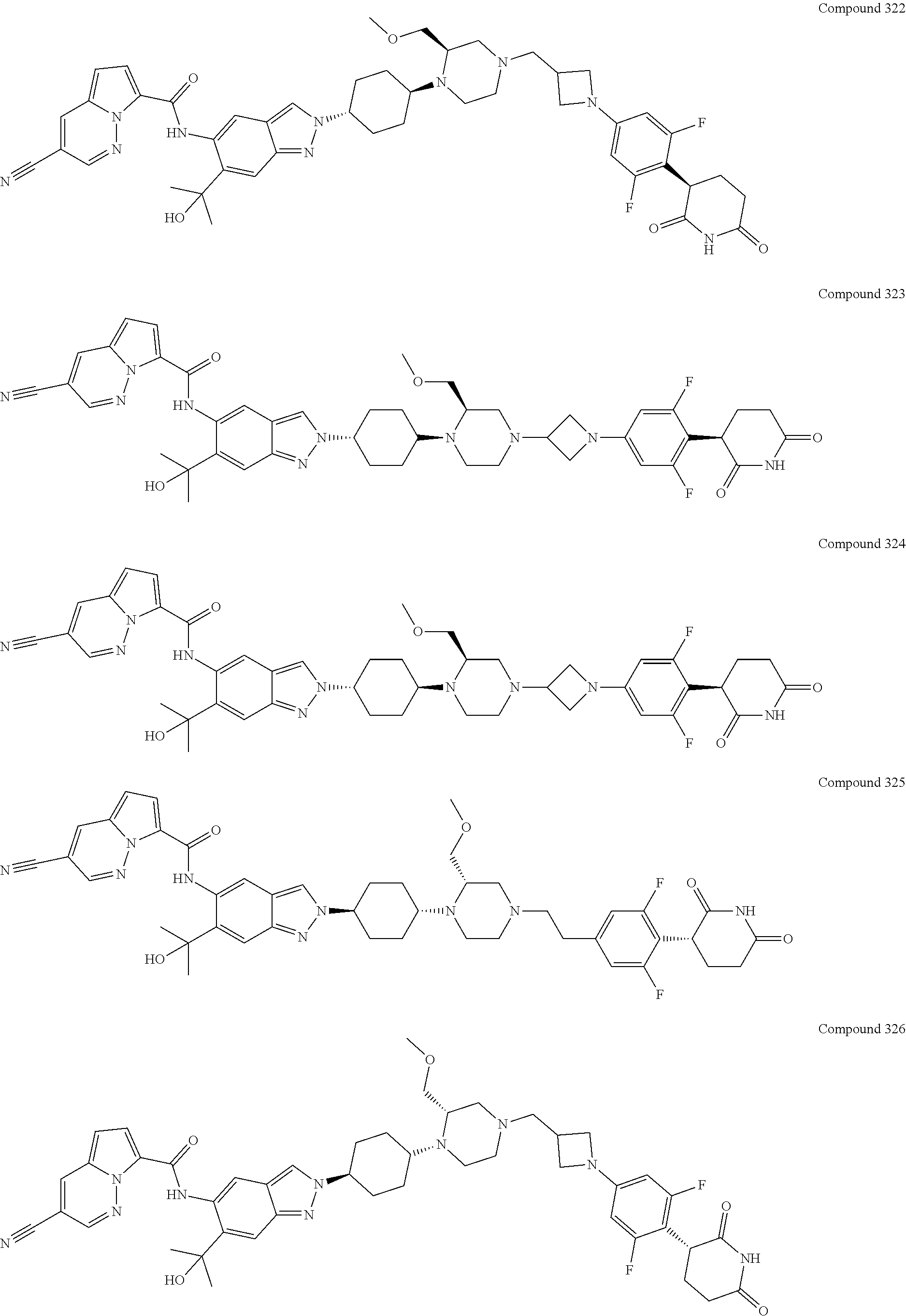

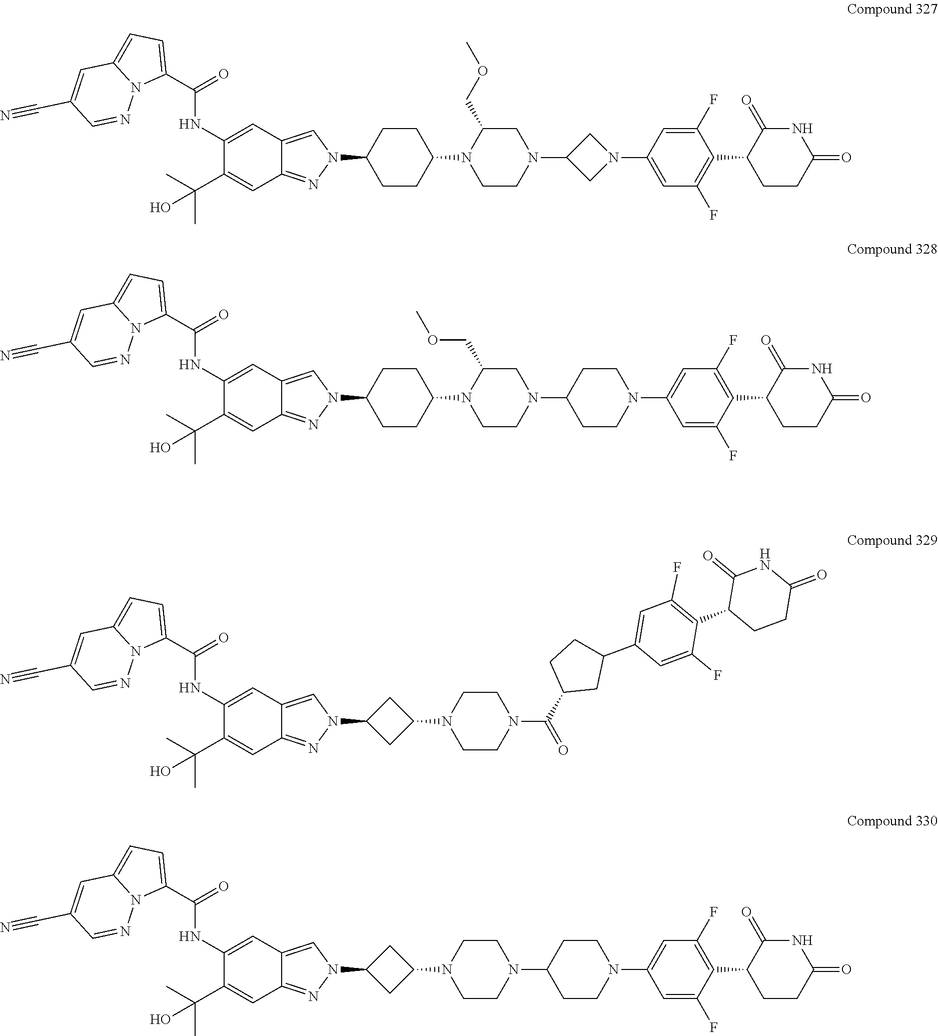

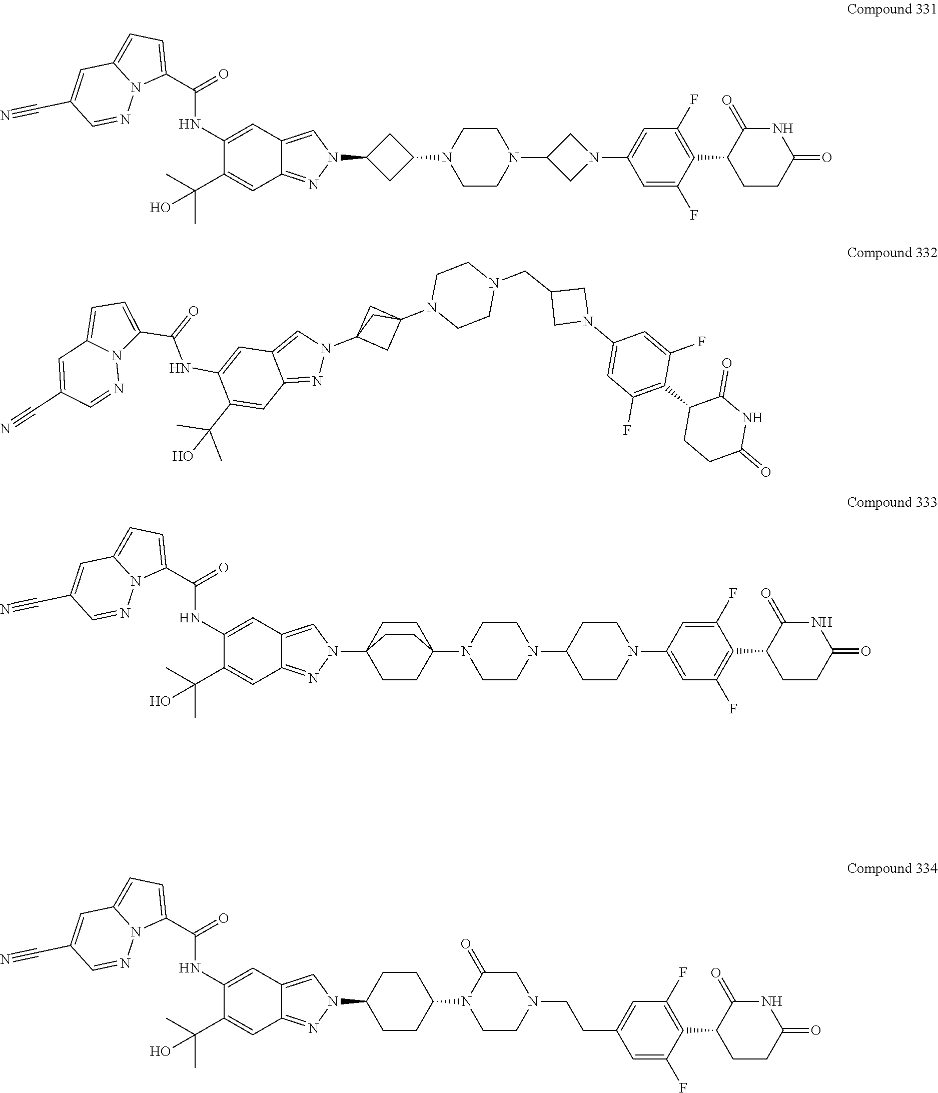 [SMILES: CC(C)(O)c1cc2nn([C@H]3C[C@H](N4CCN(C5CN(c6cc(F)c([C@H]7CCC(=O)NC7=O)c(F)c6)C5)CC4)C3)cc2cc1NC(=O)c1ccc2cc(C#N)cnn12 ; CC(C)(O)c1cc2nn(C34CC(N5CCN(CC6CN(c7cc(F)c([C@H]8CCC(=O)NC8=O)c(F)c7)C6)CC5)(C3)C4)cc2cc1NC(=O)c1ccc2cc(C#N)cnn12 ; CC(C)(O)c1cc2nn(C34CCC(N5CCN(C6CCN(c7cc(F)c([C@H]8CCC(=O)NC8=O)c(F)c7)CC6)CC5)(CC3)CC4)cc2cc1NC(=O)c1ccc2cc(C#N)cnn12 ; CC(C)(O)c1cc2nn([C@H]3CC[C@H](N4CCN(CCc5cc(F)c([C@H]6CCC(=O)NC6=O)c(F)c5)CC4=O)CC3)cc2cc1NC(=O)c1ccc2cc(C#N)cnn12]

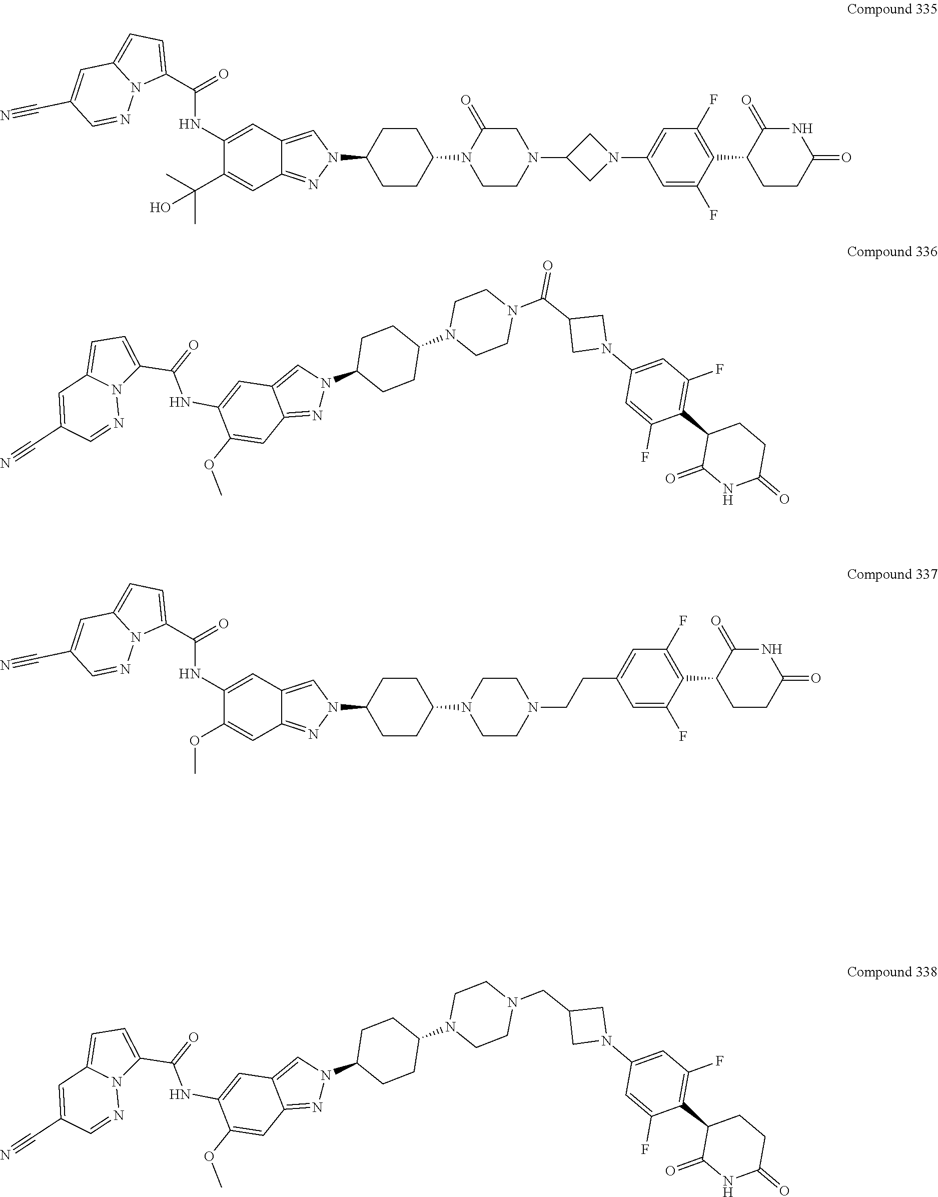 [SMILES: CC(C)(O)c1cc2nn([C@H]3CC[C@H](N4CCN(C5CN(c6cc(F)c([C@H]7CCC(=O)NC7=O)c(F)c6)C5)CC4=O)CC3)cc2cc1NC(=O)c1ccc2cc(C#N)cnn12 ; COc1cc2nn([C@H]3CC[C@H](N4CCN(C(=O)C5CN(c6cc(F)c([C@H]7CCC(=O)NC7=O)c(F)c6)C5)CC4)CC3)cc2cc1NC(=O)c1ccc2cc(C#N)cnn12 ; COc1cc2nn([C@H]3CC[C@H](N4CCN(CCc5cc(F)c([C@H]6CCC(=O)NC6=O)c(F)c5)CC4)CC3)cc2cc1NC(=O)c1ccc2cc(C#N)cnn12 ; COc1cc2nn([C@H]3CC[C@H](N4CCN(CC5CN(c6cc(F)c([C@H]7CCC(=O)NC7=O)c(F)c6)C5)CC4)CC3)cc2cc1NC(=O)c1ccc2cc(C#N)cnn12]

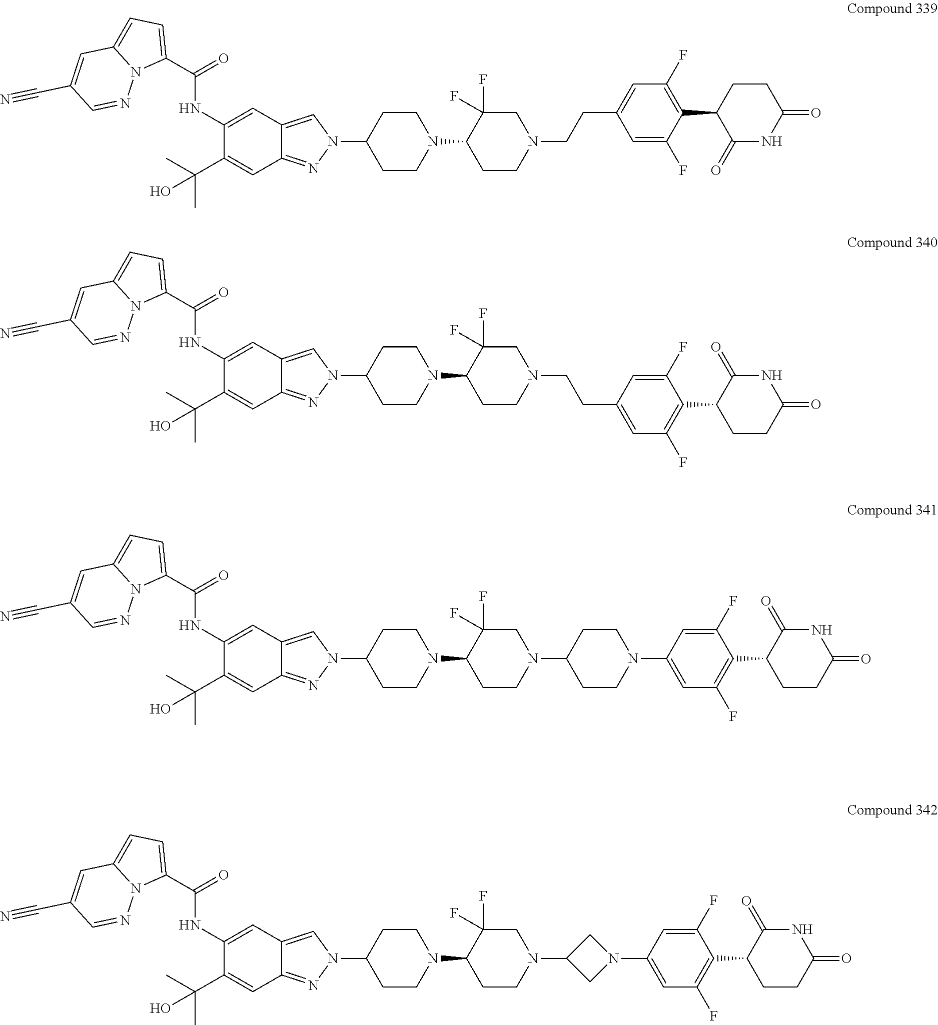 [SMILES: CC(C)(O)c1cc2nn(C3CCN([C@H]4CCN(CCc5cc(F)c([C@H]6CCC(=O)NC6=O)c(F)c5)CC4(F)F)CC3)cc2cc1NC(=O)c1ccc2cc(C#N)cnn12 ; CC(C)(O)c1cc2nn(C3CCN([C@@H]4CCN(CCc5cc(F)c([C@H]6CCC(=O)NC6=O)c(F)c5)CC4(F)F)CC3)cc2cc1NC(=O)c1ccc2cc(C#N)cnn12 ; CC(C)(O)c1cc2nn(C3CCN([C@@H]4CCN(C5CCN(c6cc(F)c([C@H]7CCC(=O)NC7=O)c(F)c6)CC5)CC4(F)F)CC3)cc2cc1NC(=O)c1ccc2cc(C#N)cnn12 ; CC(C)(O)c1cc2nn(C3CCN([C@@H]4CCN(C5CN(c6cc(F)c([C@H]7CCC(=O)NC7=O)c(F)c6)C5)CC4(F)F)CC3)cc2cc1NC(=O)c1ccc2cc(C#N)cnn12]

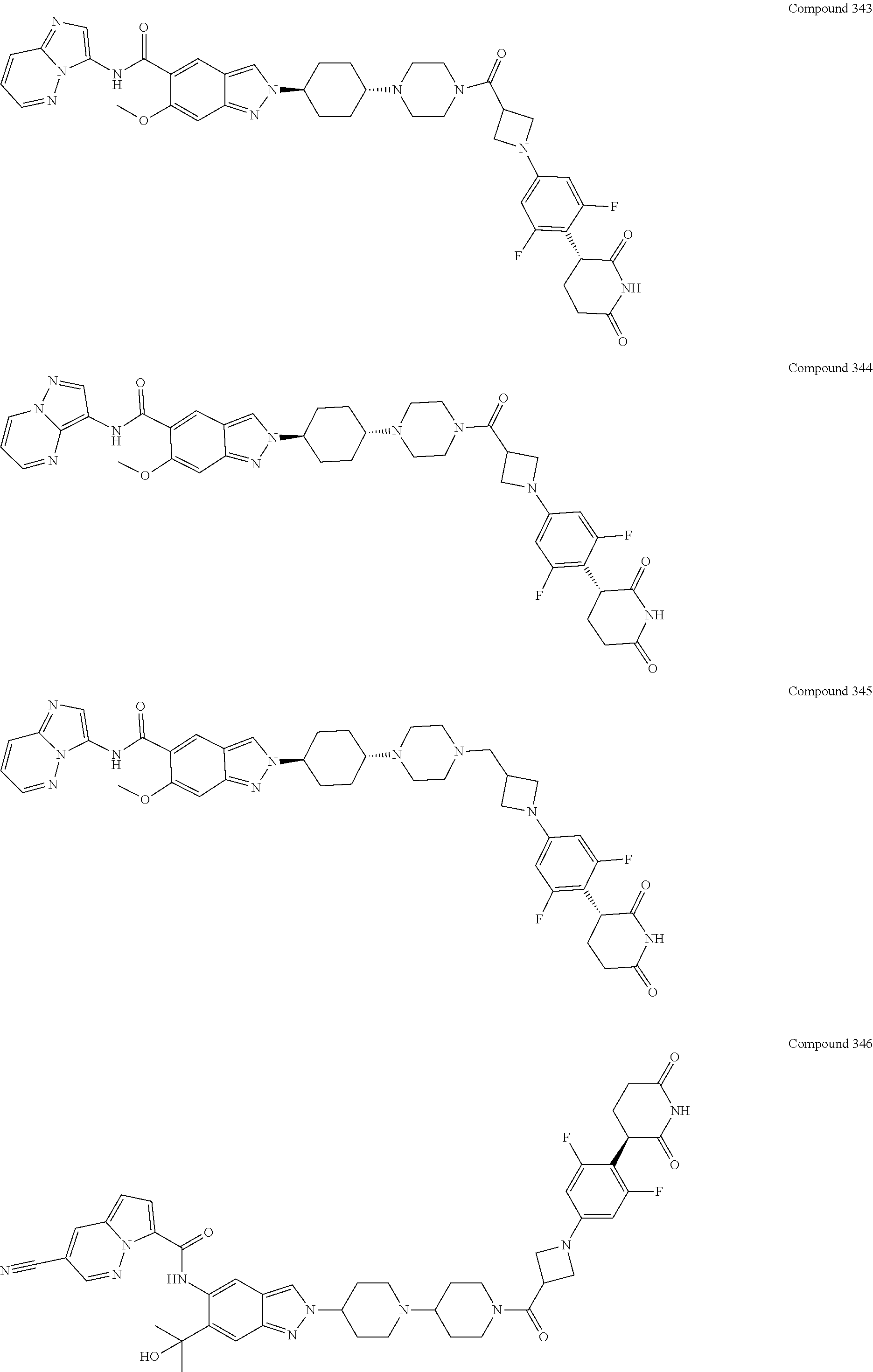 [SMILES: COc1cc2nn([C@H]3CC[C@H](N4CCN(C(=O)C5CN(c6cc(F)c([C@H]7CCC(=O)NC7=O)c(F)c6)C5)CC4)CC3)cc2cc1C(=O)Nc1cnc2cccnn12 ; COc1cc2nn([C@H]3CC[C@H](N4CCN(C(=O)C5CN(c6cc(F)c([C@H]7CCC(=O)NC7=O)c(F)c6)C5)CC4)CC3)cc2cc1C(=O)Nc1cnn2cccnc12 ; COc1cc2nn([C@H]3CC[C@H](N4CCN(CC5CN(c6cc(F)c([C@H]7CCC(=O)NC7=O)c(F)c6)C5)CC4)CC3)cc2cc1C(=O)Nc1cnc2cccnn12 ; CC(C)(O)c1cc2nn(C3CCN(C4CCN(C(=O)C5CN(c6cc(F)c([C@H]7CCC(=O)NC7=O)c(F)c6)C5)CC4)CC3)cc2cc1NC(=O)c1ccc2cc(C#N)cnn12]

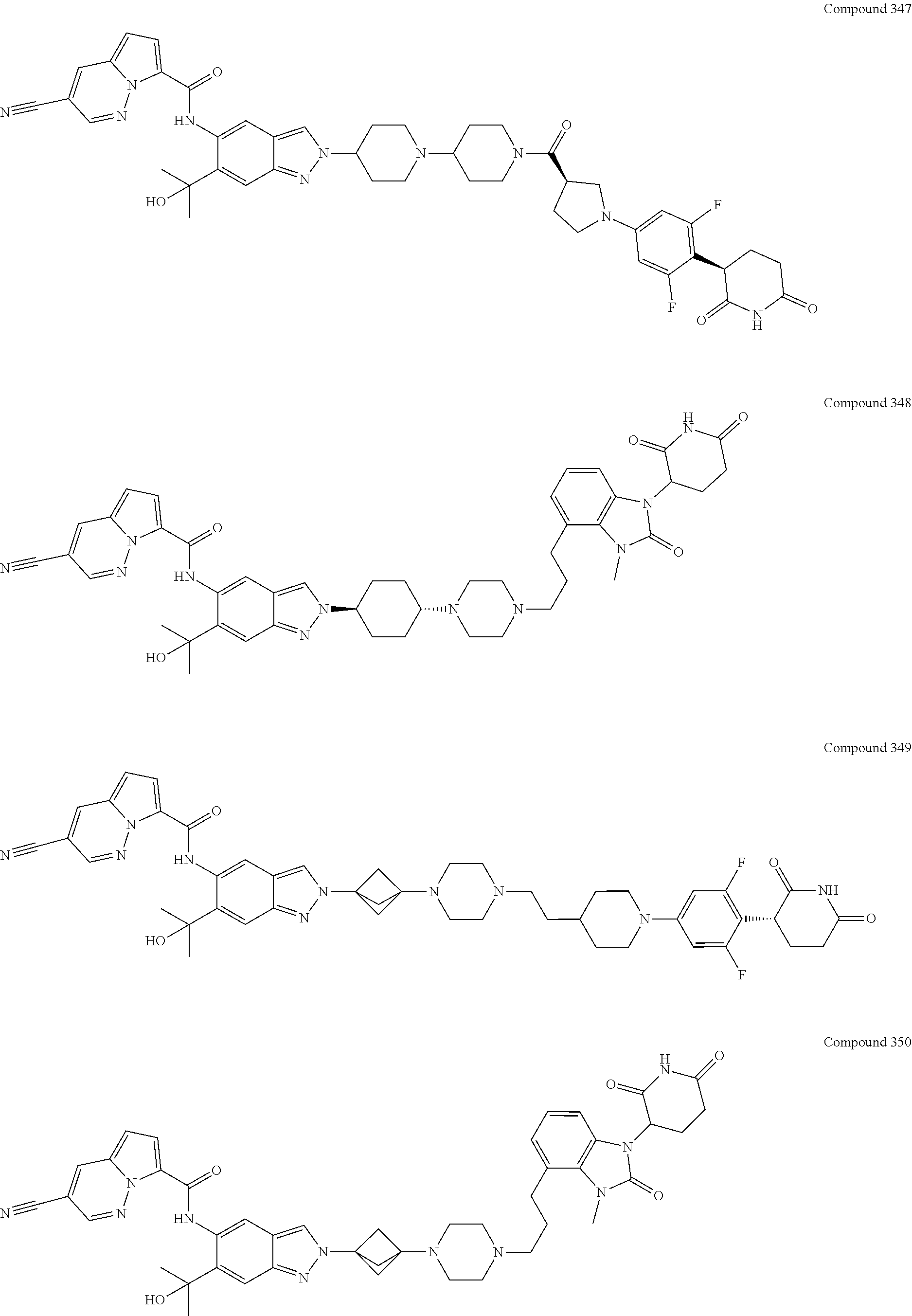 [SMILES: CC(C)(O)c1cc2nn(C3CCN(C4CCN(C(=O)[C@@H]5CCN(c6cc(F)c([C@H]7CCC(=O)NC7=O)c(F)c6)C5)CC4)CC3)cc2cc1NC(=O)c1ccc2cc(C#N)cnn12 ; Cn1c(=O)n(C2CCC(=O)NC2=O)c2cccc(CCCN3CCN([C@H]4CC[C@H](n5cc6cc(NC(=O)c7ccc8cc(C#N)cnn78)c(C(C)(C)O)cc6n5)CC4)CC3)c21 ; CC(C)(O)c1cc2nn(C34CC(N5CCN(CCC6CCN(c7cc(F)c([C@H]8CCC(=O)NC8=O)c(F)c7)CC6)CC5)(C3)C4)cc2cc1NC(=O)c1ccc2cc(C#N)cnn12 ; Cn1c(=O)n(C2CCC(=O)NC2=O)c2cccc(CCCN3CCN(C45CC(n6cc7cc(NC(=O)c8ccc9cc(C#N)cnn89)c(C(C)(C)O)cc7n6)(C4)C5)CC3)c21]

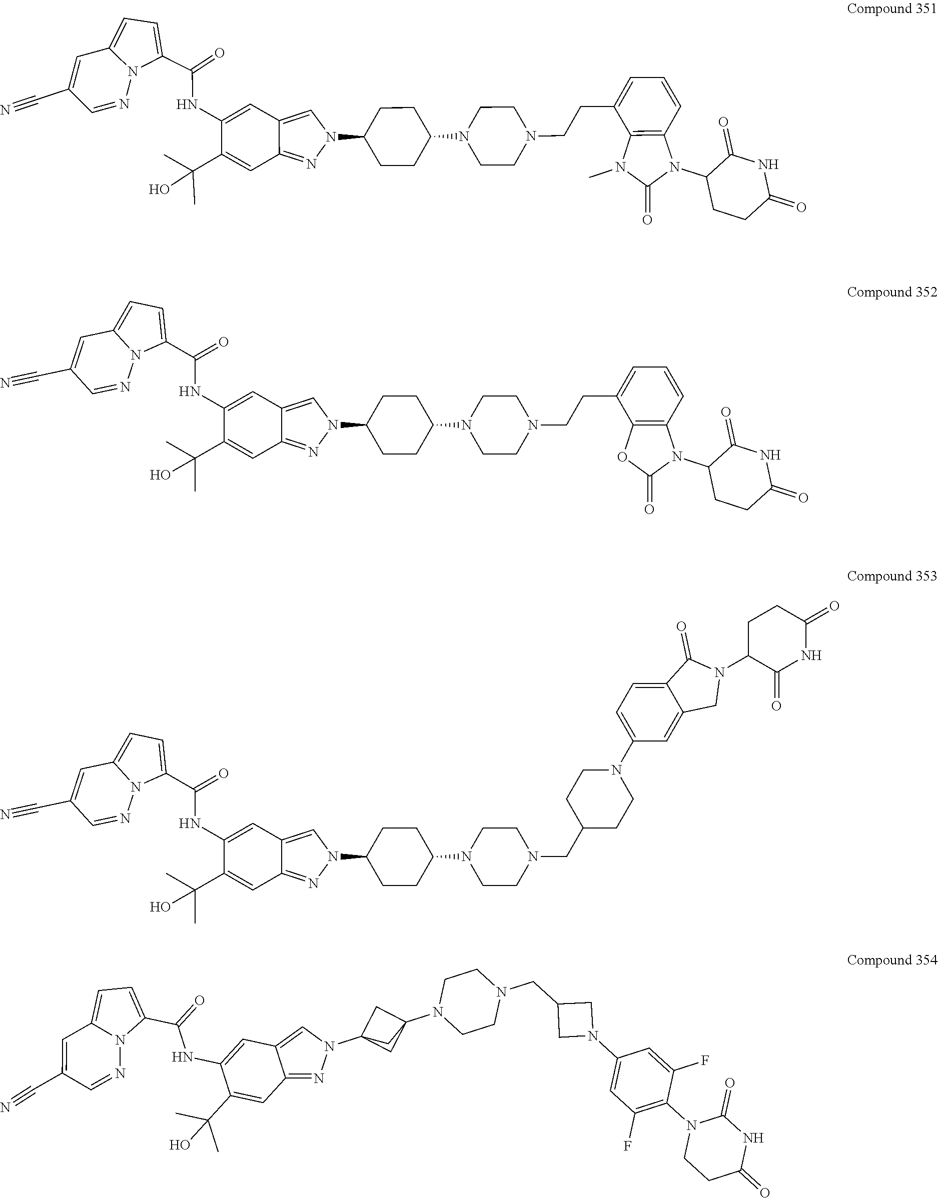 [SMILES: Cn1c(=O)n(C2CCC(=O)NC2=O)c2cccc(CCN3CCN([C@H]4CC[C@H](n5cc6cc(NC(=O)c7ccc8cc(C#N)cnn78)c(C(C)(C)O)cc6n5)CC4)CC3)c21 ; CC(C)(O)c1cc2nn([C@H]3CC[C@H](N4CCN(CCc5cccc6c5oc(=O)n6C5CCC(=O)NC5=O)CC4)CC3)cc2cc1NC(=O)c1ccc2cc(C#N)cnn12 ; CC(C)(O)c1cc2nn([C@H]3CC[C@H](N4CCN(CC5CCN(c6ccc7c(c6)CN(C6CCC(=O)NC6=O)C7=O)CC5)CC4)CC3)cc2cc1NC(=O)c1ccc2cc(C#N)cnn12 ; CC(C)(O)c1cc2nn(C34CC(N5CCN(CC6CN(c7cc(F)c(N8CCC(=O)NC8=O)c(F)c7)C6)CC5)(C3)C4)cc2cc1NC(=O)c1ccc2cc(C#N)cnn12]

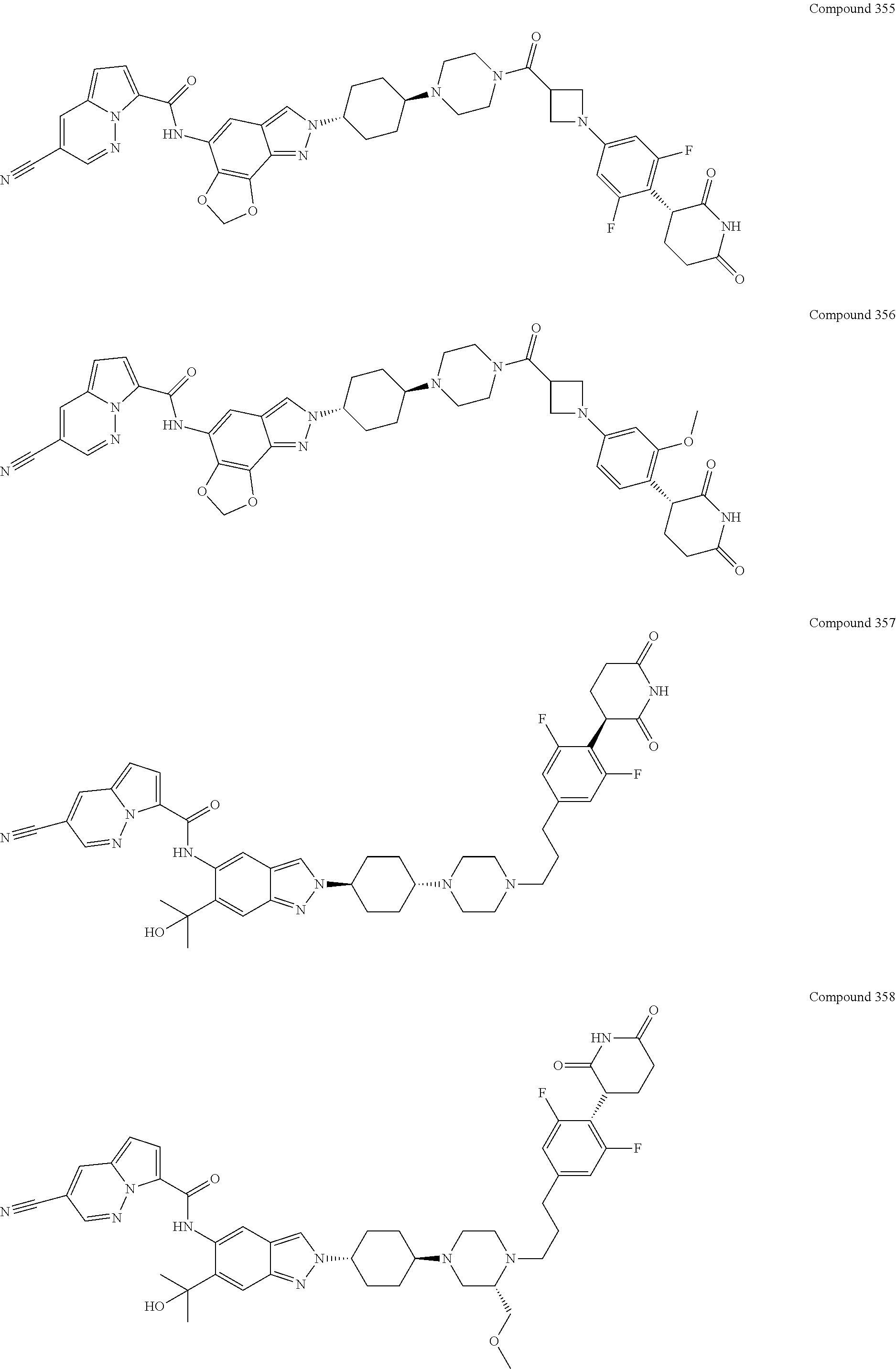

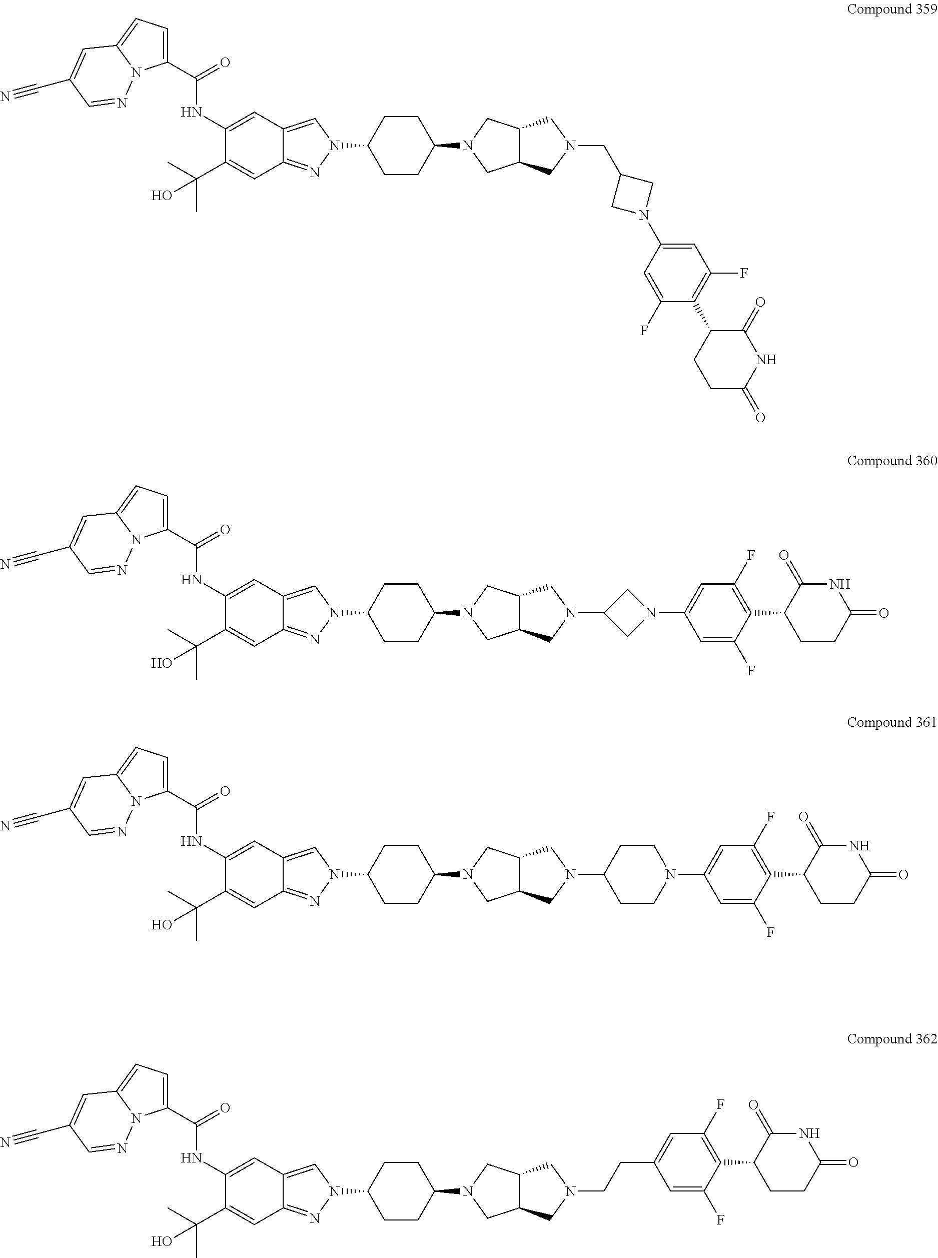

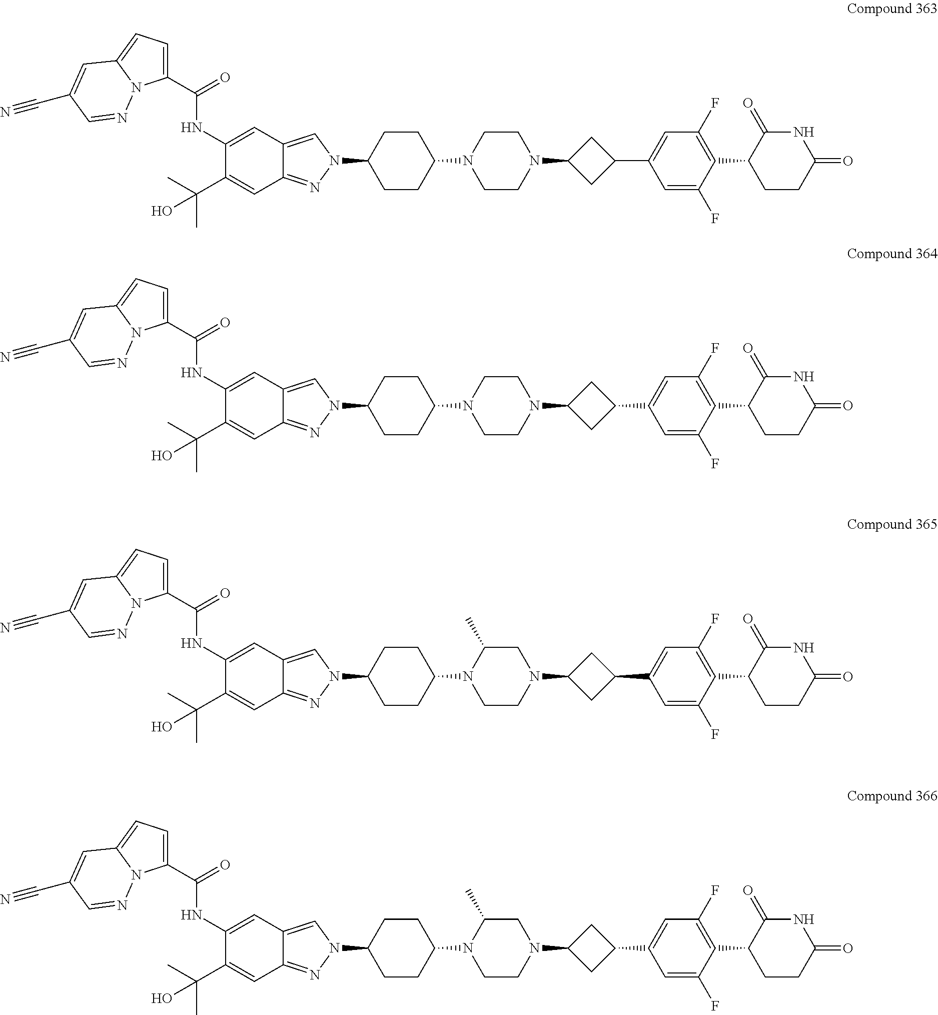

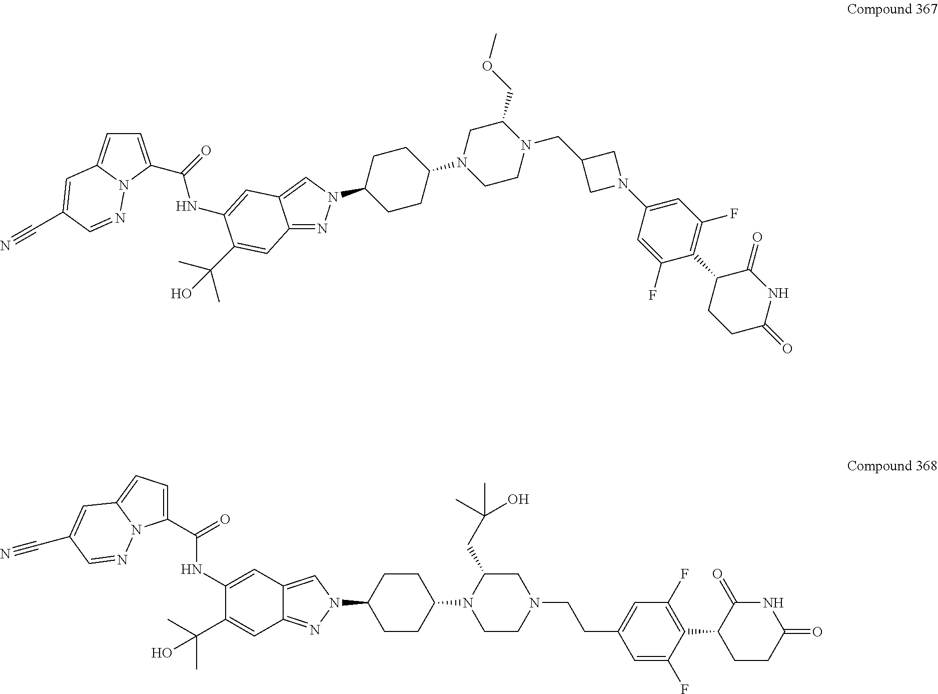 [SMILES: COC[C@H]1CN([C@H]2CC[C@H](n3cc4cc(NC(=O)c5ccc6cc(C#N)cnn56)c(C(C)(C)O)cc4n3)CC2)CCN1CC1CN(c2cc(F)c([C@H]3CCC(=O)NC3=O)c(F)c2)C1 ; CC(C)(O)C[C@@H]1CN(CCc2cc(F)c([C@H]3CCC(=O)NC3=O)c(F)c2)CCN1[C@H]1CC[C@H](n2cc3cc(NC(=O)c4ccc5cc(C#N)cnn45)c(C(C)(C)O)cc3n2)CC1]

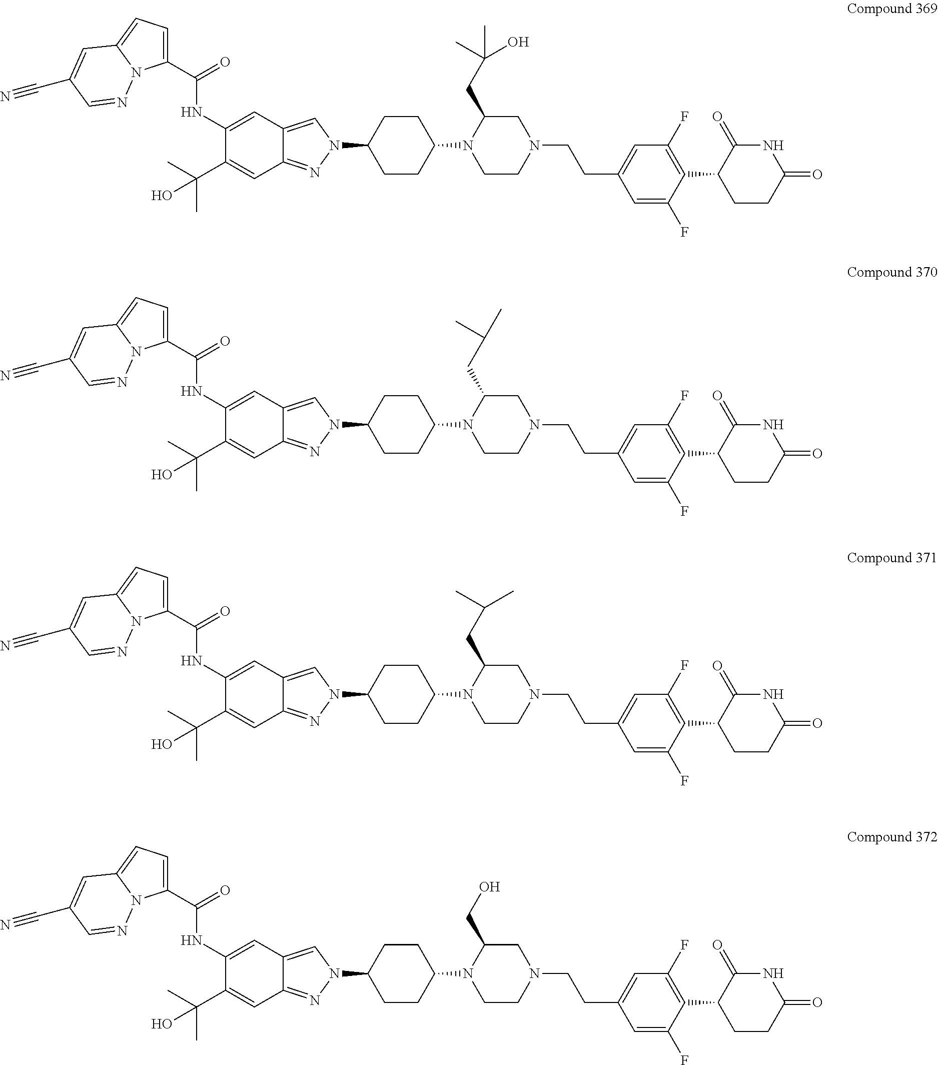 [SMILES: CC(C)(O)C[C@H]1CN(CCc2cc(F)c([C@H]3CCC(=O)NC3=O)c(F)c2)CCN1[C@H]1CC[C@H](n2cc3cc(NC(=O)c4ccc5cc(C#N)cnn45)c(C(C)(C)O)cc3n2)CC1 ; CC(C)C[C@@H]1CN(CCc2cc(F)c([C@H]3CCC(=O)NC3=O)c(F)c2)CCN1[C@H]1CC[C@H](n2cc3cc(NC(=O)c4ccc5cc(C#N)cnn45)c(C(C)(C)O)cc3n2)CC1 ; CC(C)C[C@H]1CN(CCc2cc(F)c([C@H]3CCC(=O)NC3=O)c(F)c2)CCN1[C@H]1CC[C@H](n2cc3cc(NC(=O)c4ccc5cc(C#N)cnn45)c(C(C)(C)O)cc3n2)CC1 ; CC(C)(O)c1cc2nn([C@H]3CC[C@H](N4CCN(CCc5cc(F)c([C@H]6CCC(=O)NC6=O)c(F)c5)C[C@@H]4CO)CC3)cc2cc1NC(=O)c1ccc2cc(C#N)cnn12]

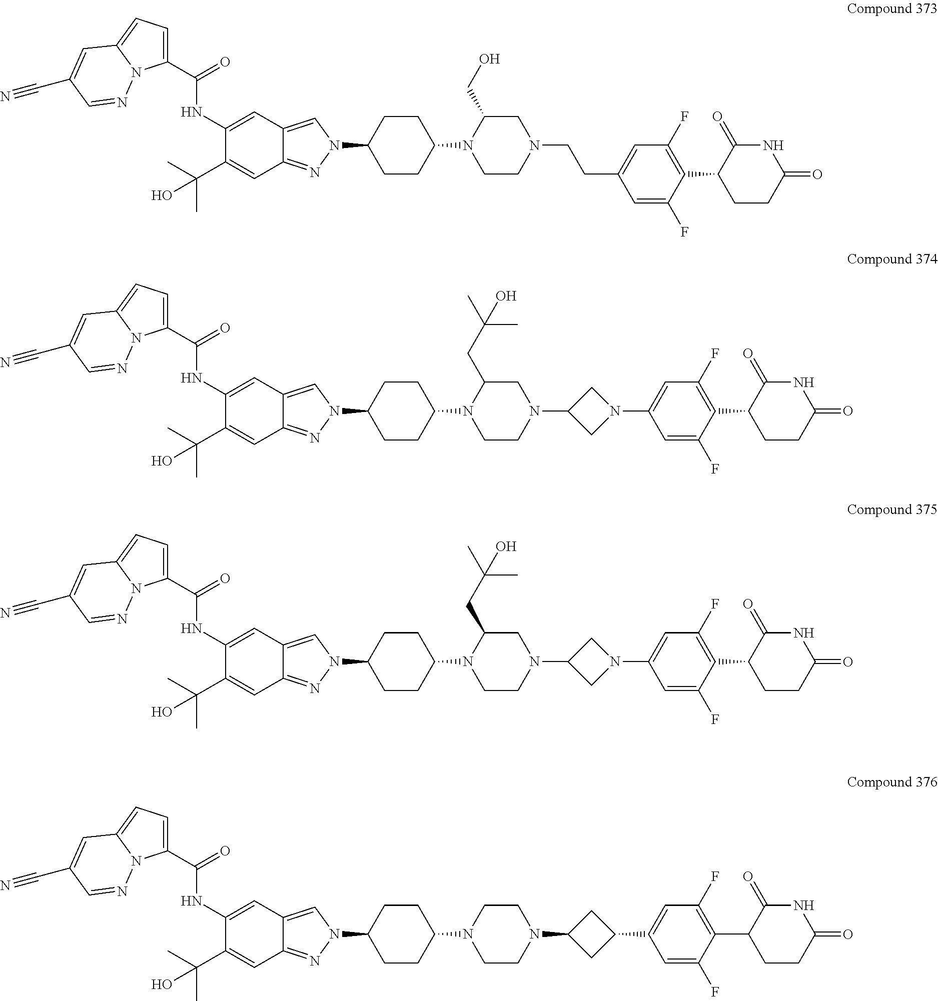

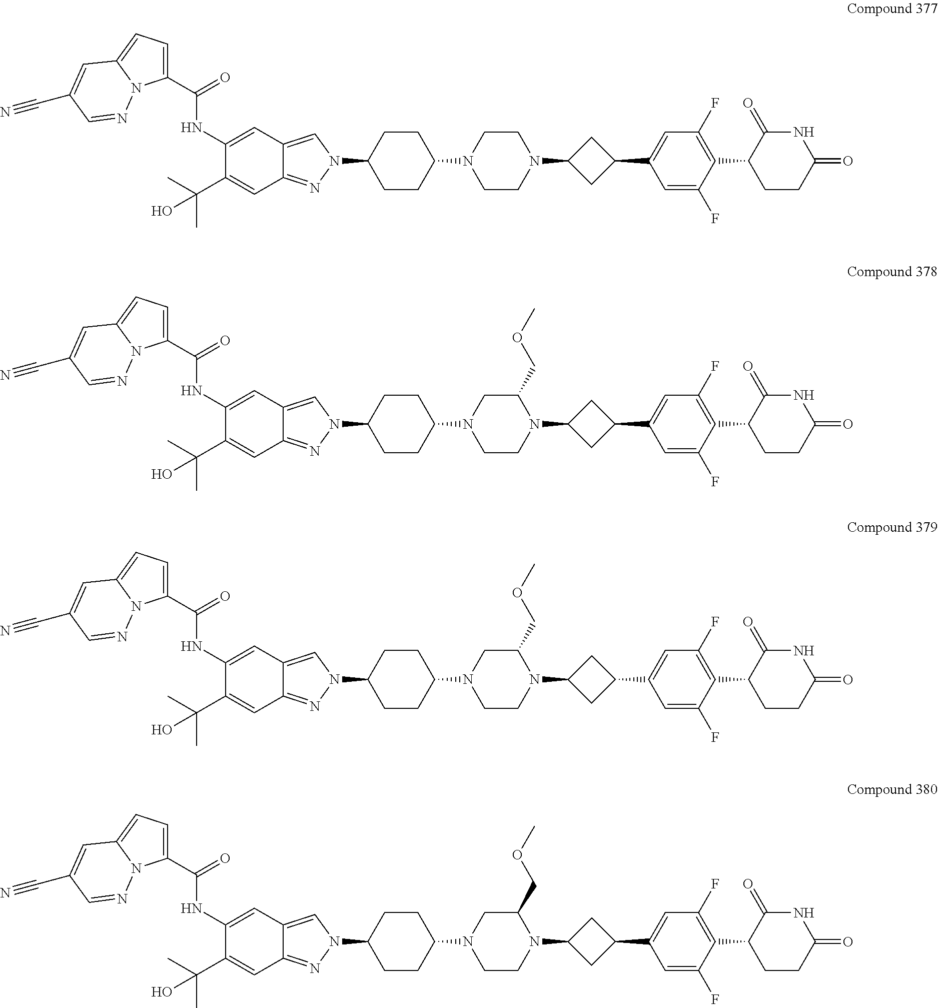

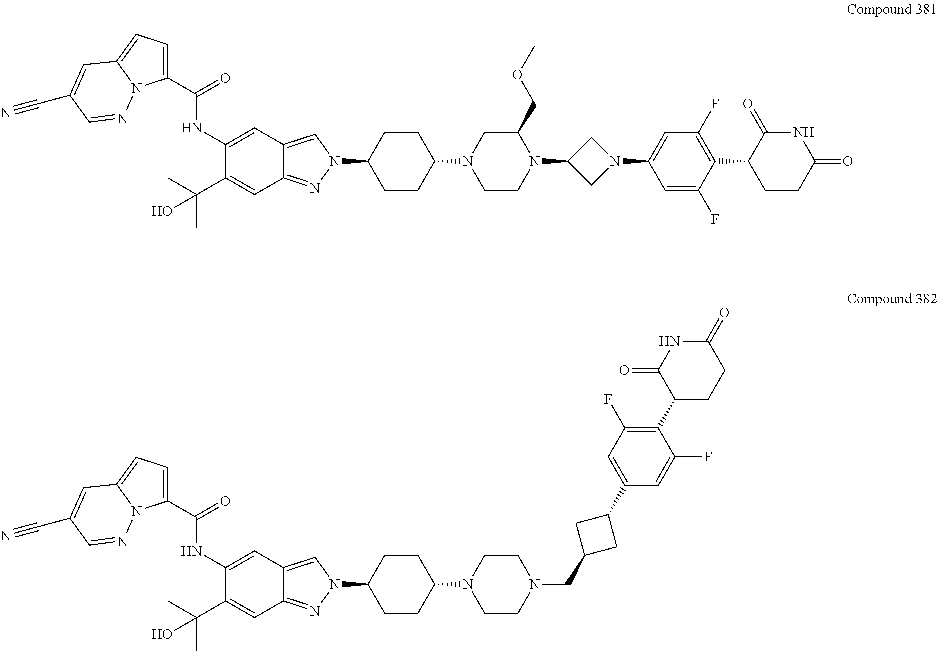

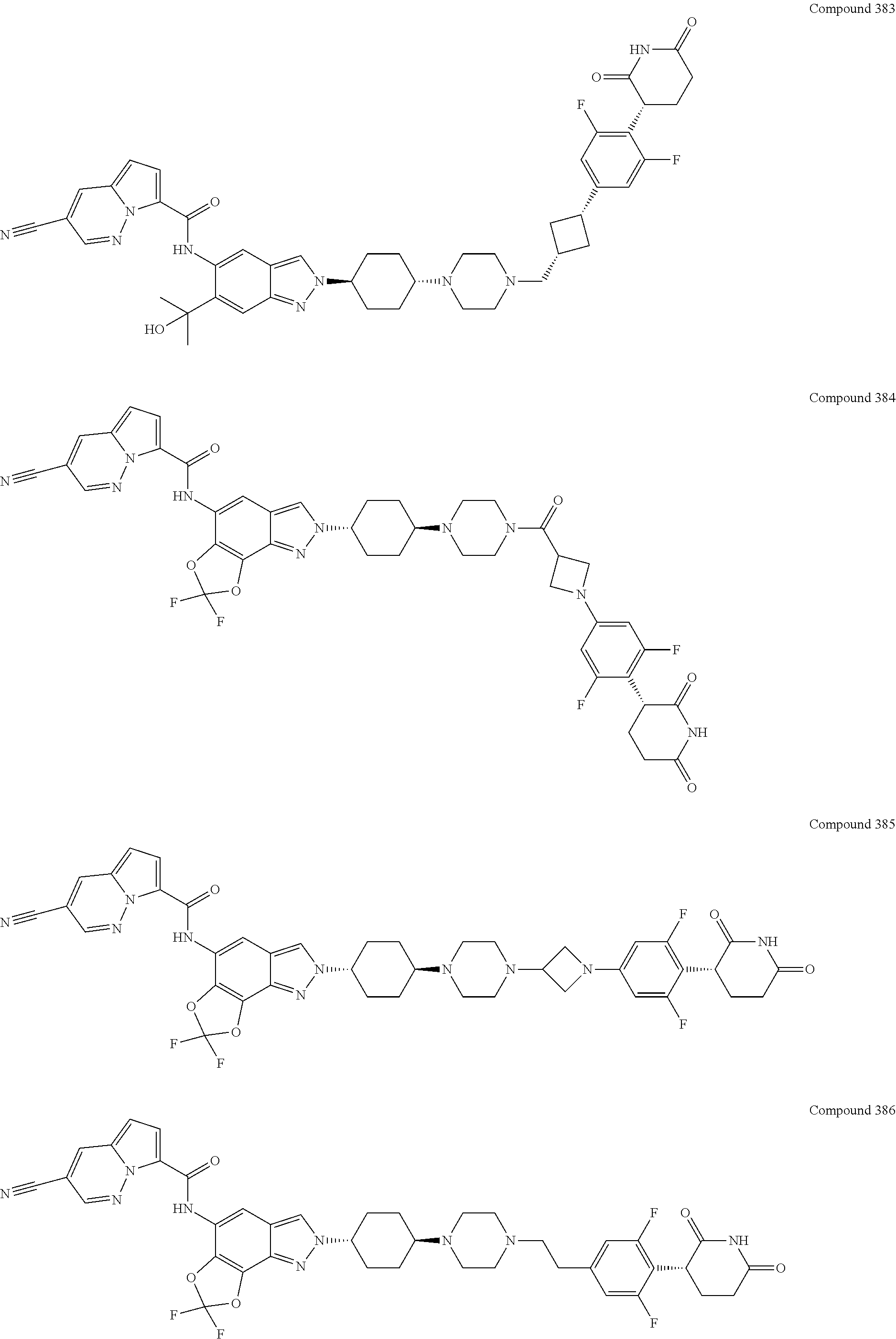 [SMILES: CC(C)(O)c1cc2nn([C@H]3CC[C@H](N4CCN(C[C@H]5C[C@@H](c6cc(F)c([C@H]7CCC(=O)NC7=O)c(F)c6)C5)CC4)CC3)cc2cc1NC(=O)c1ccc2cc(C#N)cnn12 ; N#Cc1cnn2c(C(=O)Nc3cc4cn([C@H]5CC[C@H](N6CCN(C(=O)C7CN(c8cc(F)c([C@H]9CCC(=O)NC9=O)c(F)c8)C7)CC6)CC5)nc4c4c3OC(F)(F)O4)ccc2c1 ; N#Cc1cnn2c(C(=O)Nc3cc4cn([C@H]5CC[C@H](N6CCN(C7CN(c8cc(F)c([C@H]9CCC(=O)NC9=O)c(F)c8)C7)CC6)CC5)nc4c4c3OC(F)(F)O4)ccc2c1 ; N#Cc1cnn2c(C(=O)Nc3cc4cn([C@H]5CC[C@H](N6CCN(CCc7cc(F)c([C@H]8CCC(=O)NC8=O)c(F)c7)CC6)CC5)nc4c4c3OC(F)(F)O4)ccc2c1]

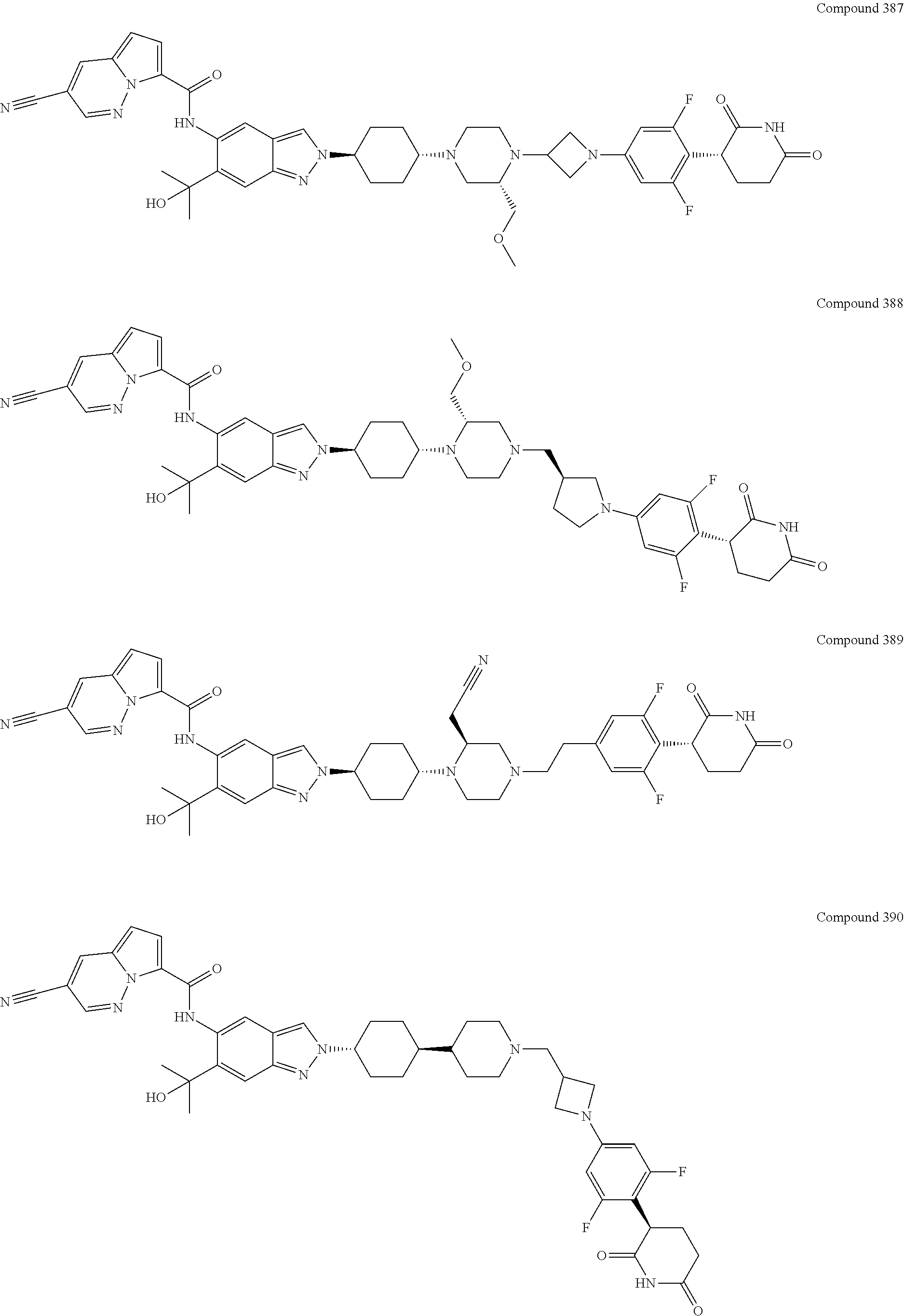

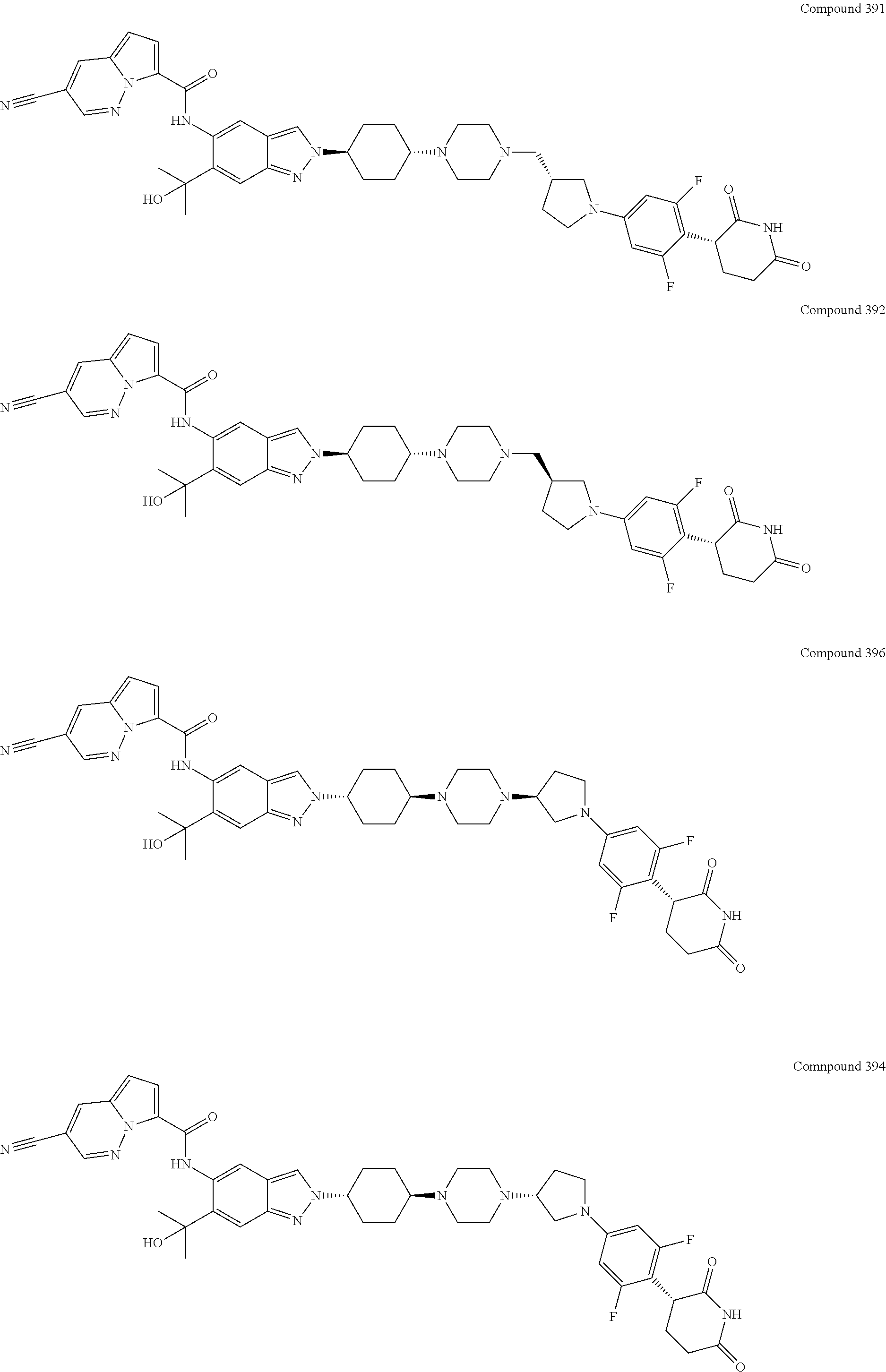

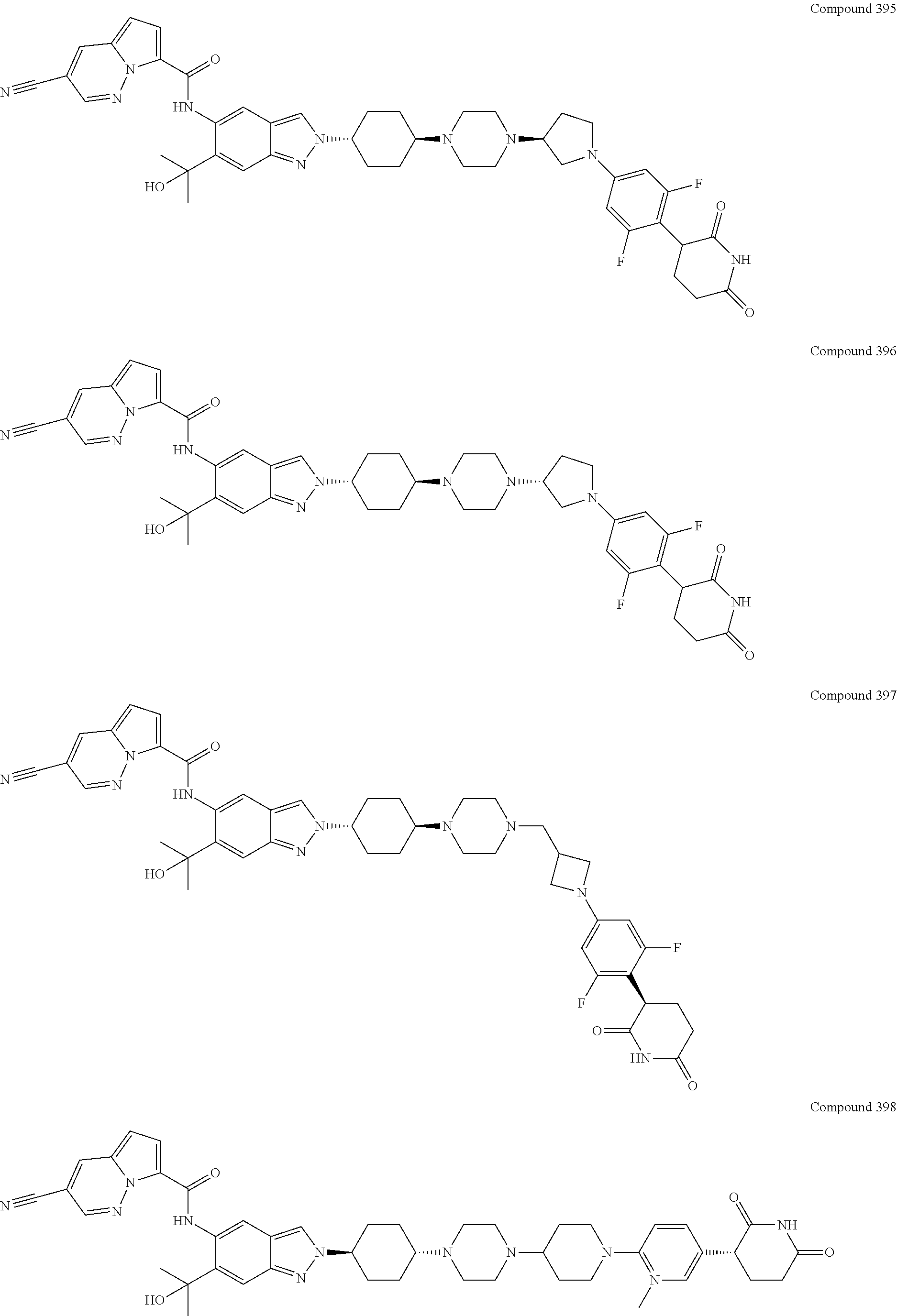

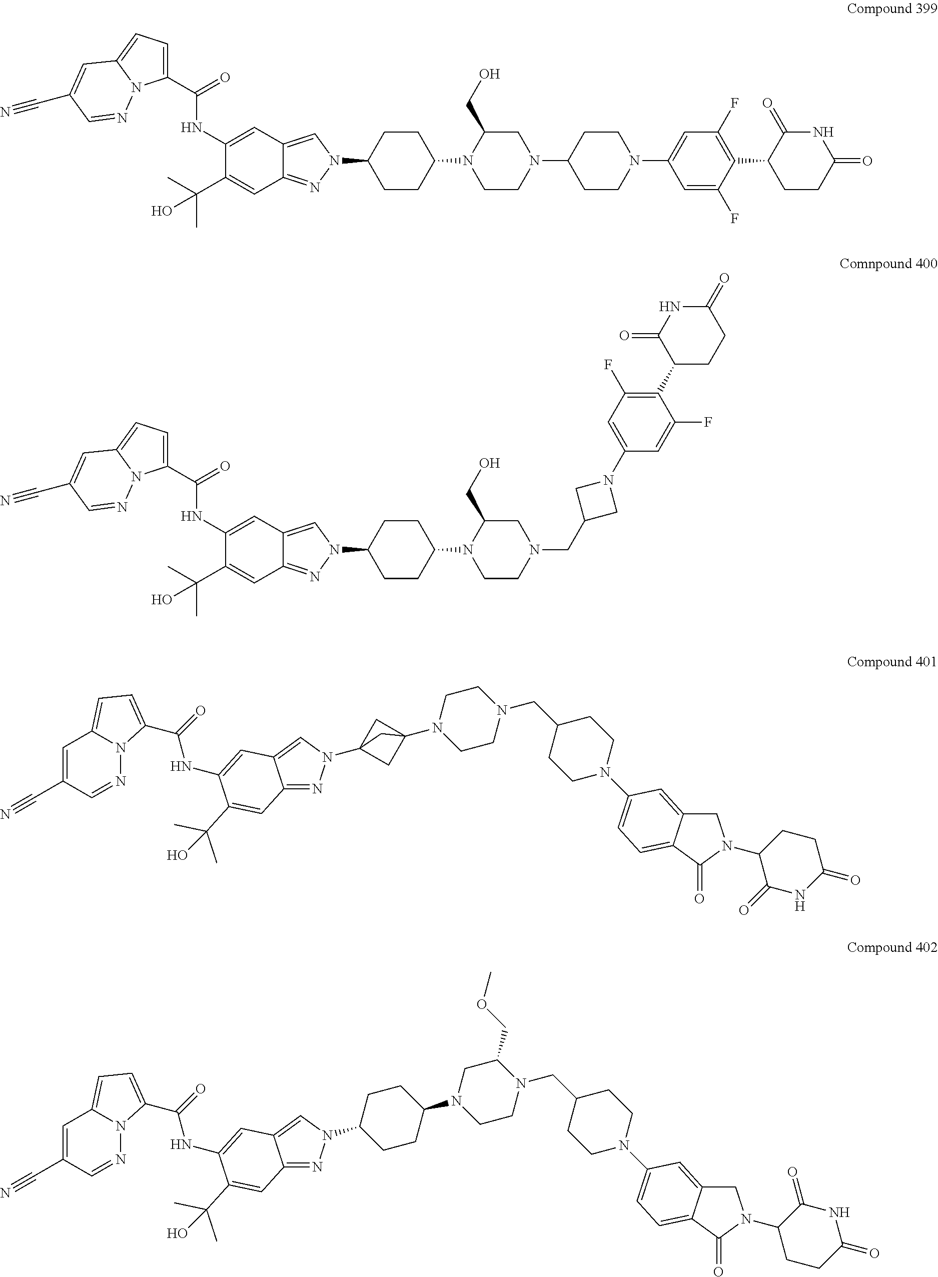

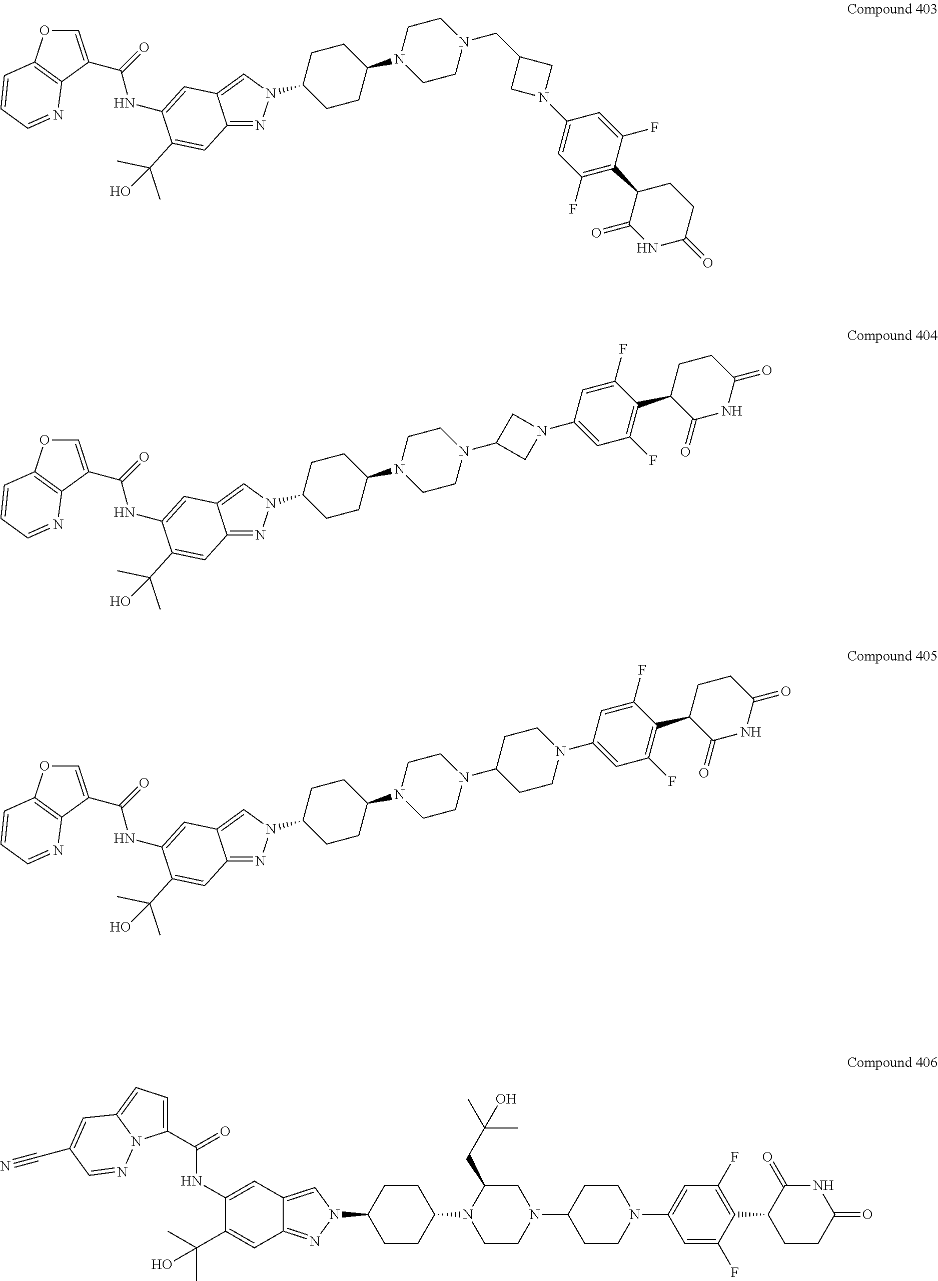 [SMILES: CC(C)(O)c1cc2nn([C@H]3CC[C@H](N4CCN(CC5CN(c6cc(F)c([C@H]7CCC(=O)NC7=O)c(F)c6)C5)CC4)CC3)cc2cc1NC(=O)c1coc2cccnc12 ; CC(C)(O)c1cc2nn([C@H]3CC[C@H](N4CCN(C5CN(c6cc(F)c([C@H]7CCC(=O)NC7=O)c(F)c6)C5)CC4)CC3)cc2cc1NC(=O)c1coc2cccnc12 ; CC(C)(O)c1cc2nn([C@H]3CC[C@H](N4CCN(C5CCN(c6cc(F)c([C@H]7CCC(=O)NC7=O)c(F)c6)CC5)CC4)CC3)cc2cc1NC(=O)c1coc2cccnc12 ; CC(C)(O)C[C@H]1CN(C2CCN(c3cc(F)c([C@H]4CCC(=O)NC4=O)c(F)c3)CC2)CCN1[C@H]1CC[C@H](n2cc3cc(NC(=O)c4ccc5cc(C#N)cnn45)c(C(C)(C)O)cc3n2)CC1]

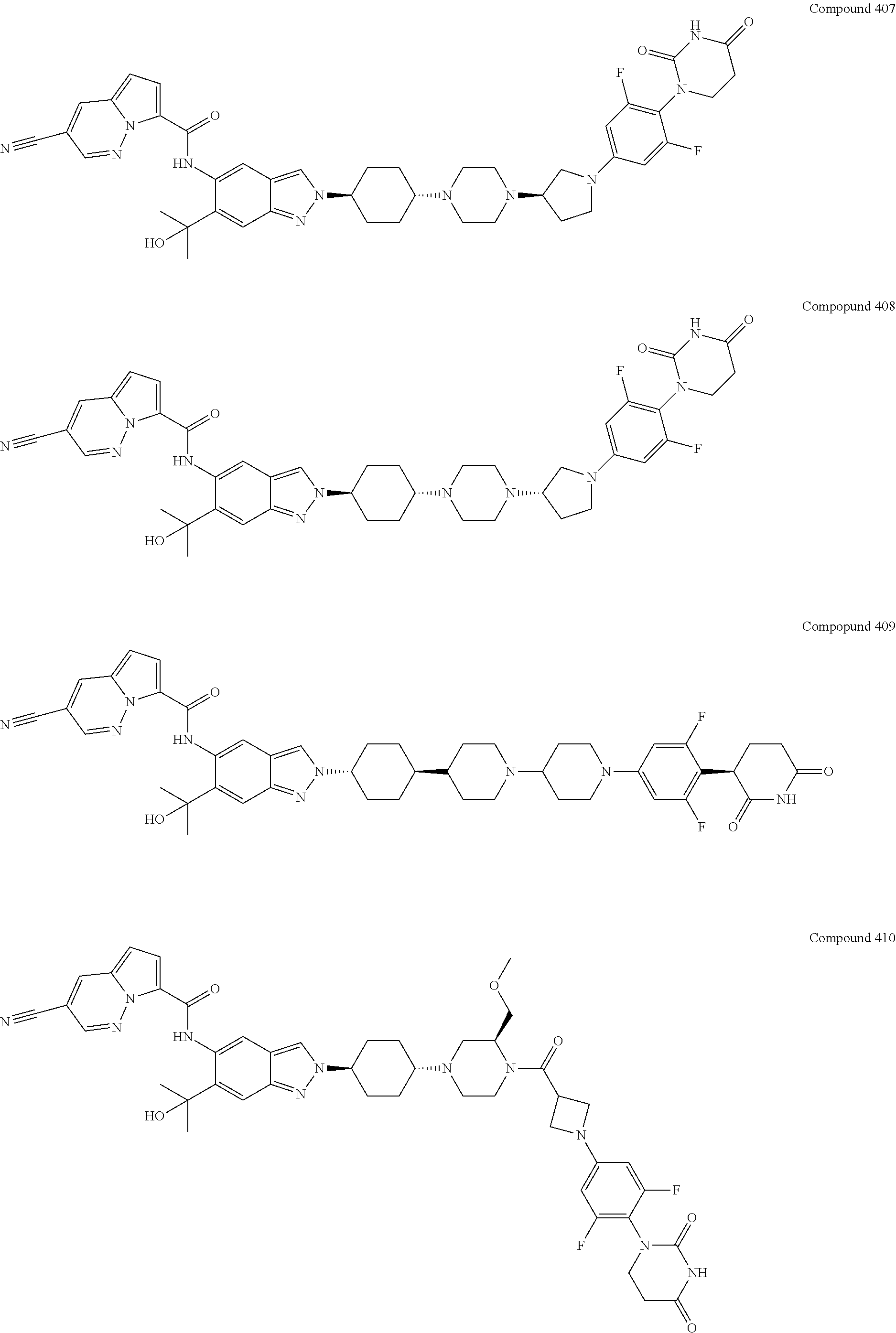 [SMILES: CC(C)(O)c1cc2nn([C@H]3CC[C@H](N4CCN([C@@H]5CCN(c6cc(F)c(N7CCC(=O)NC7=O)c(F)c6)C5)CC4)CC3)cc2cc1NC(=O)c1ccc2cc(C#N)cnn12 ; CC(C)(O)c1cc2nn([C@H]3CC[C@H](N4CCN([C@H]5CCN(c6cc(F)c(N7CCC(=O)NC7=O)c(F)c6)C5)CC4)CC3)cc2cc1NC(=O)c1ccc2cc(C#N)cnn12 ; CC(C)(O)c1cc2nn([C@H]3CC[C@H](C4CCN(C5CCN(c6cc(F)c([C@H]7CCC(=O)NC7=O)c(F)c6)CC5)CC4)CC3)cc2cc1NC(=O)c1ccc2cc(C#N)cnn12 ; COC[C@@H]1CN([C@H]2CC[C@H](n3cc4cc(NC(=O)c5ccc6cc(C#N)cnn56)c(C(C)(C)O)cc4n3)CC2)CCN1C(=O)C1CN(c2cc(F)c(N3CCC(=O)NC3=O)c(F)c2)C1]

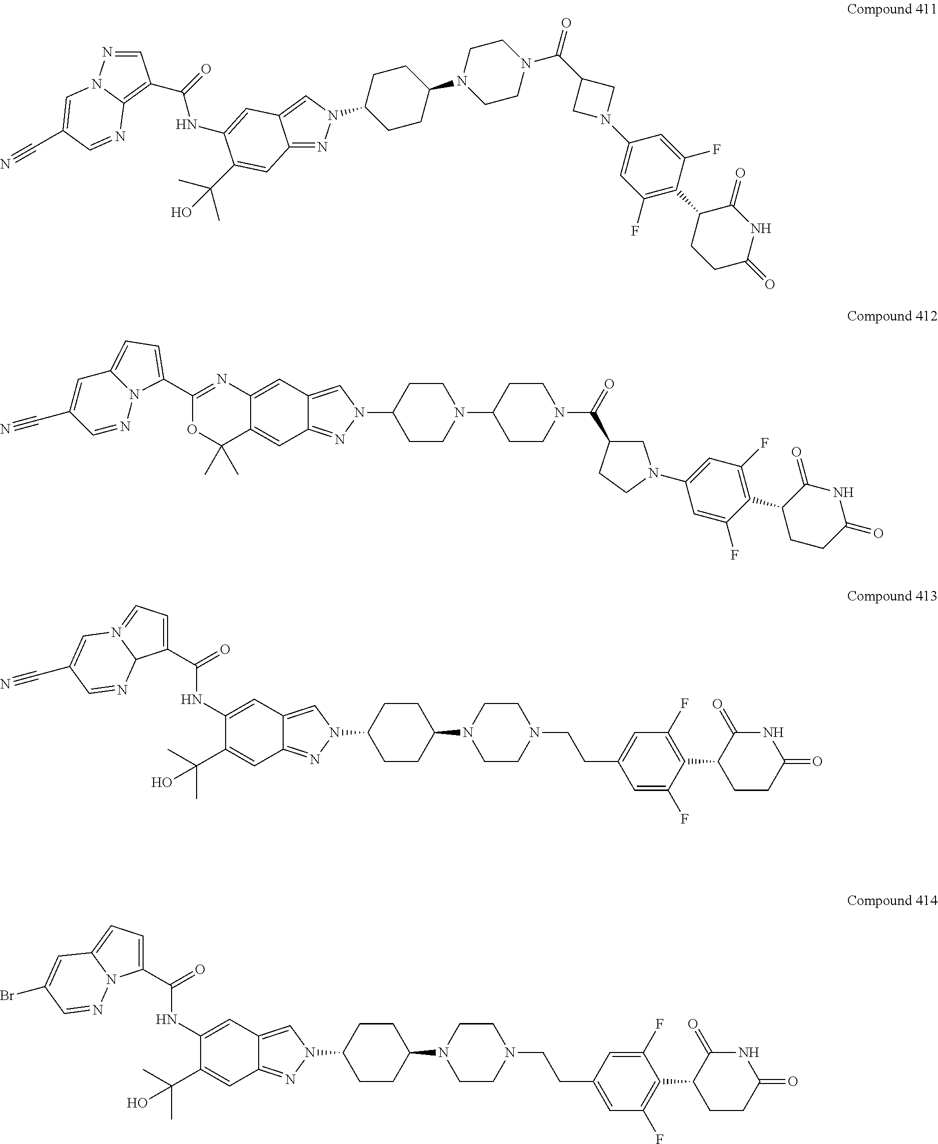

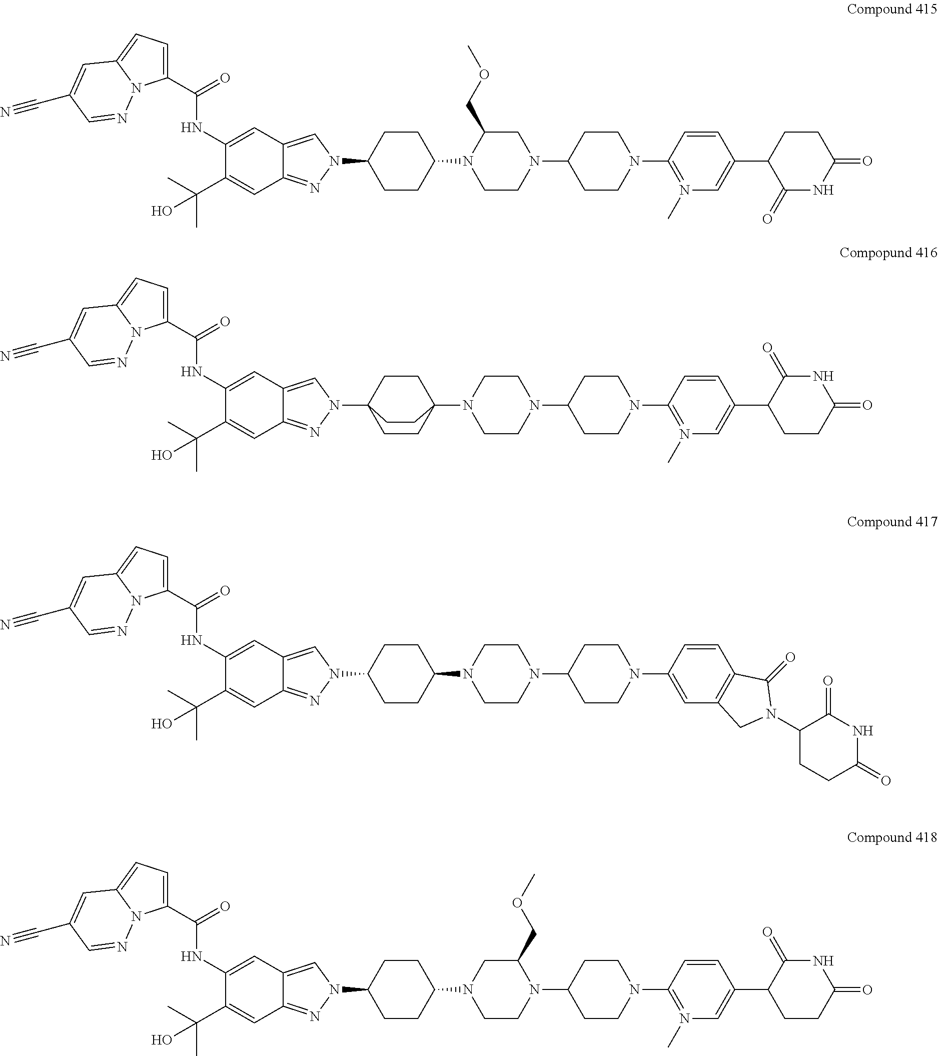

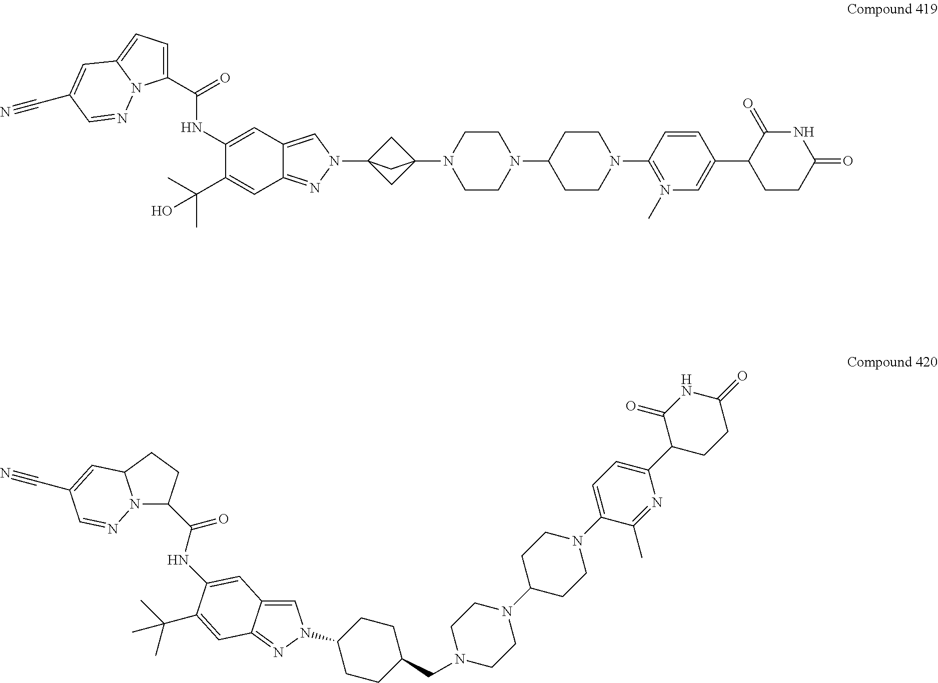

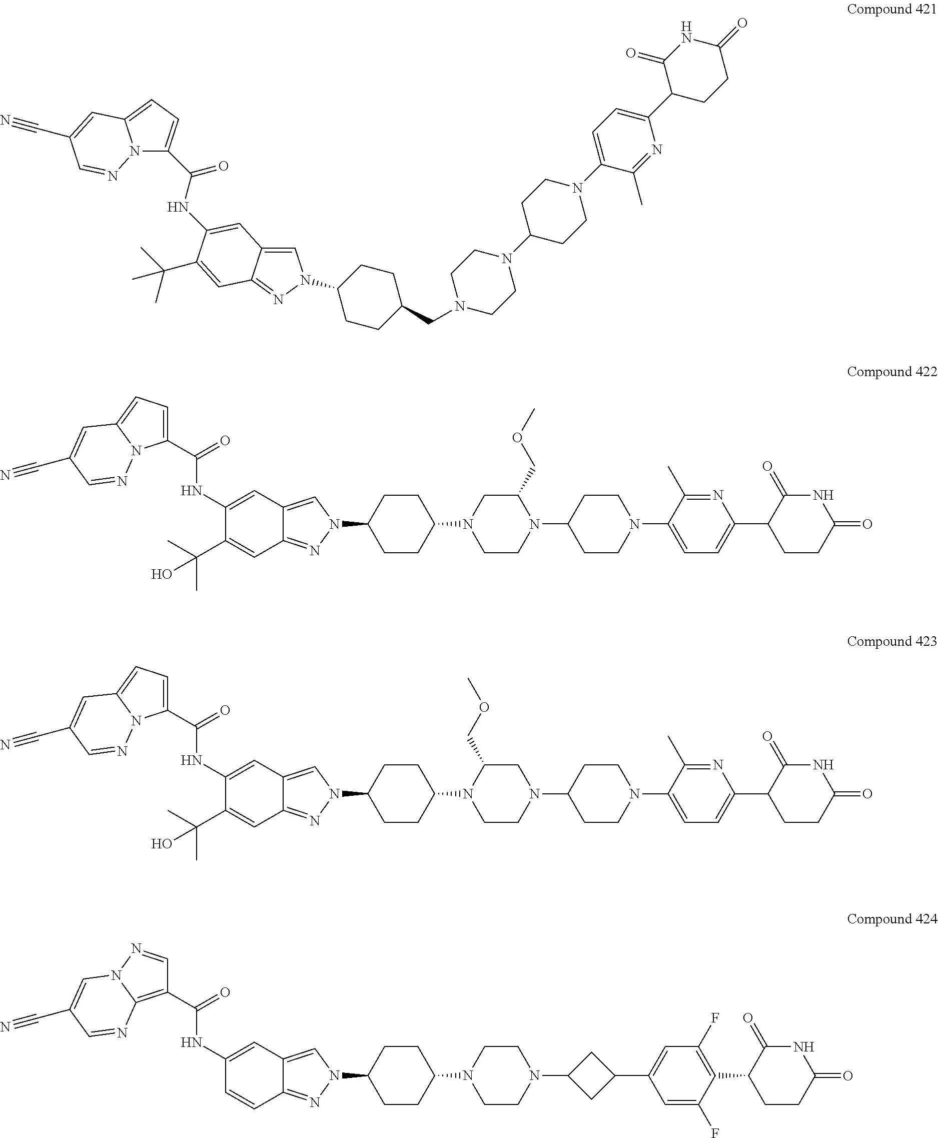 [SMILES: Cc1nc(C2CCC(=O)NC2=O)ccc1N1CCC(N2CCN(C[C@H]3CC[C@H](n4cc5cc(NC(=O)c6ccc7cc(C#N)cnn67)c(C(C)(C)C)cc5n4)CC3)CC2)CC1 ; COC[C@H]1CN([C@H]2CC[C@H](n3cc4cc(NC(=O)c5ccc6cc(C#N)cnn56)c(C(C)(C)O)cc4n3)CC2)CCN1C1CCN(c2ccc(C3CCC(=O)NC3=O)nc2C)CC1 ; COC[C@@H]1CN(C2CCN(c3ccc(C4CCC(=O)NC4=O)nc3C)CC2)CCN1[C@H]1CC[C@H](n2cc3cc(NC(=O)c4ccc5cc(C#N)cnn45)c(C(C)(C)O)cc3n2)CC1 ; N#Cc1cnc2c(C(=O)Nc3ccc4nn([C@H]5CC[C@H](N6CCN(C7CC(c8cc(F)c([C@H]9CCC(=O)NC9=O)c(F)c8)C7)CC6)CC5)cc4c3)cnn2c1]

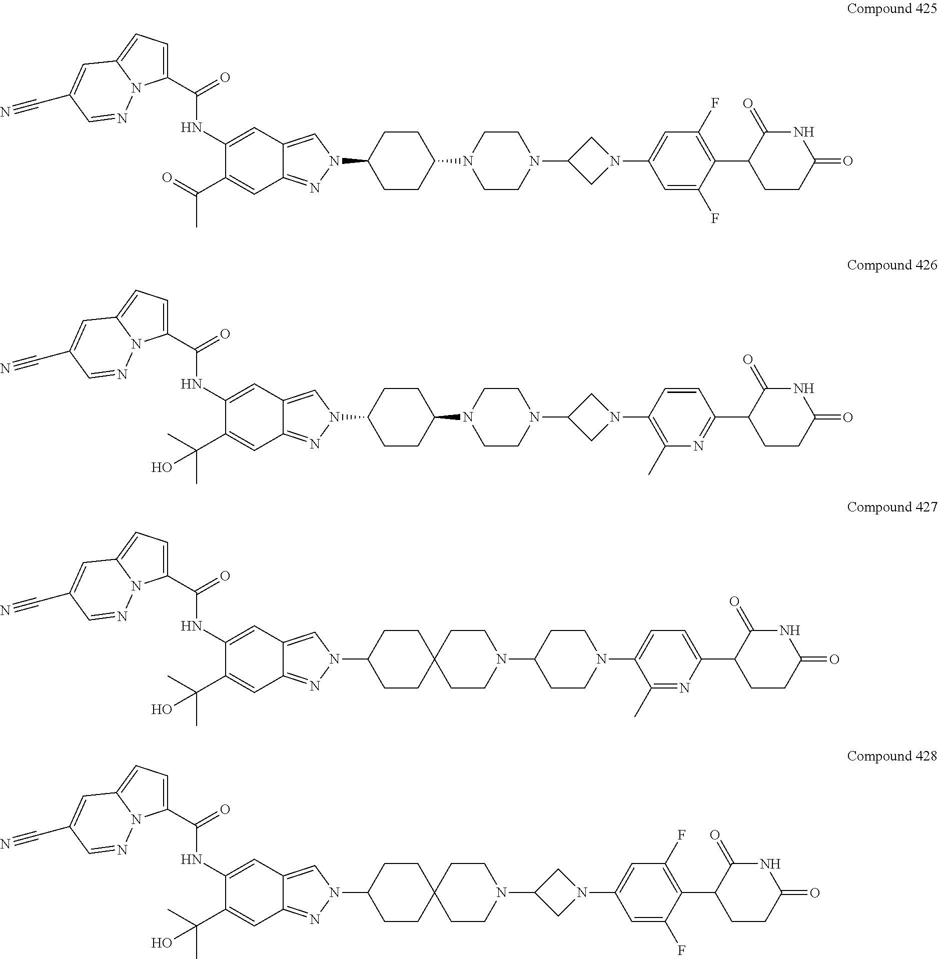 [SMILES: CC(=O)c1cc2nn([C@H]3CC[C@H](N4CCN(C5CN(c6cc(F)c(C7CCC(=O)NC7=O)c(F)c6)C5)CC4)CC3)cc2cc1NC(=O)c1ccc2cc(C#N)cnn12 ; Cc1nc(C2CCC(=O)NC2=O)ccc1N1CC(N2CCN([C@H]3CC[C@H](n4cc5cc(NC(=O)c6ccc7cc(C#N)cnn67)c(C(C)(C)O)cc5n4)CC3)CC2)C1 ; Cc1nc(C2CCC(=O)NC2=O)ccc1N1CCC(N2CCC3(CCC(n4cc5cc(NC(=O)c6ccc7cc(C#N)cnn67)c(C(C)(C)O)cc5n4)CC3)CC2)CC1 ; CC(C)(O)c1cc2nn(C3CCC4(CC3)CCN(C3CN(c5cc(F)c(C6CCC(=O)NC6=O)c(F)c5)C3)CC4)cc2cc1NC(=O)c1ccc2cc(C#N)cnn12]

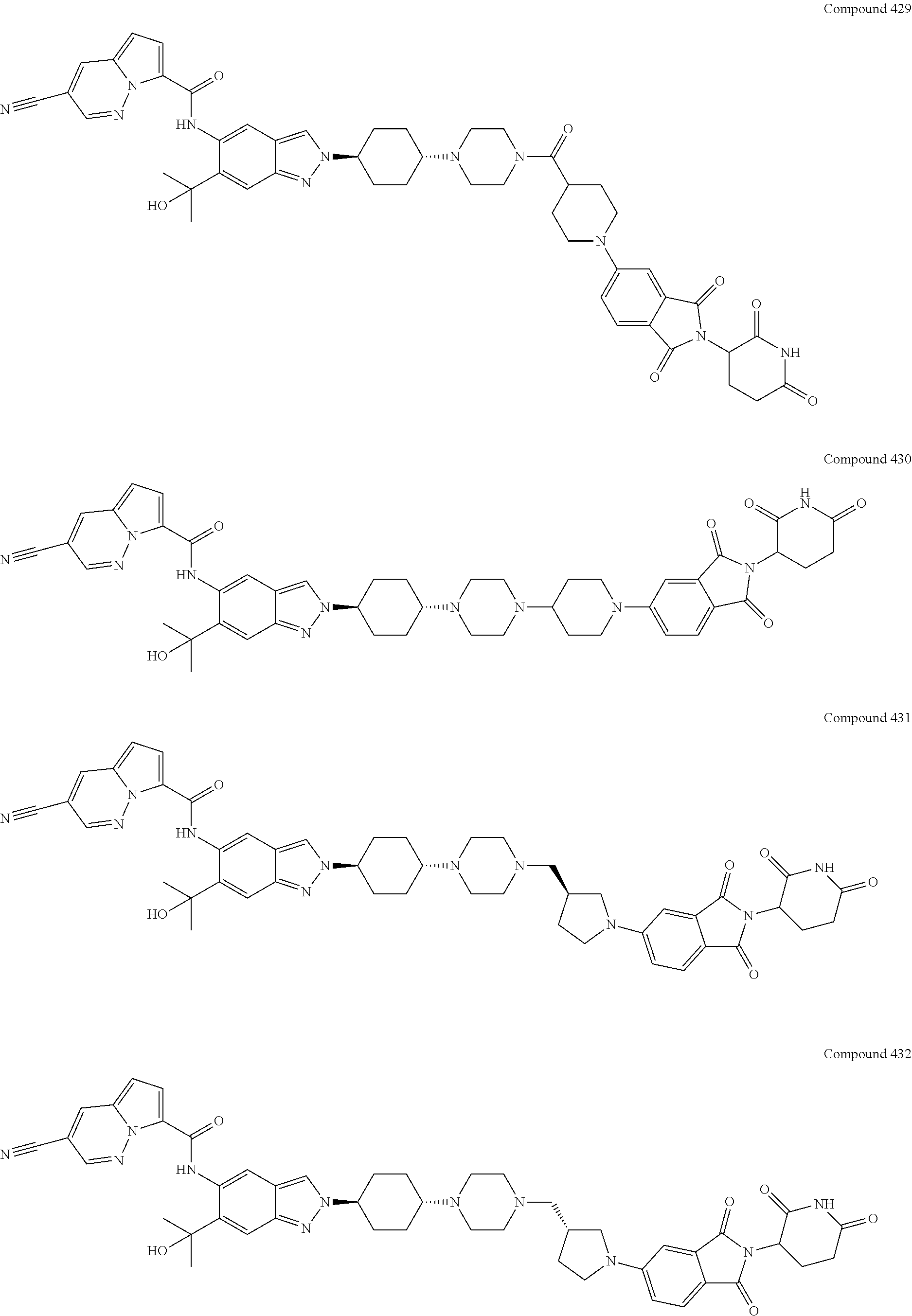

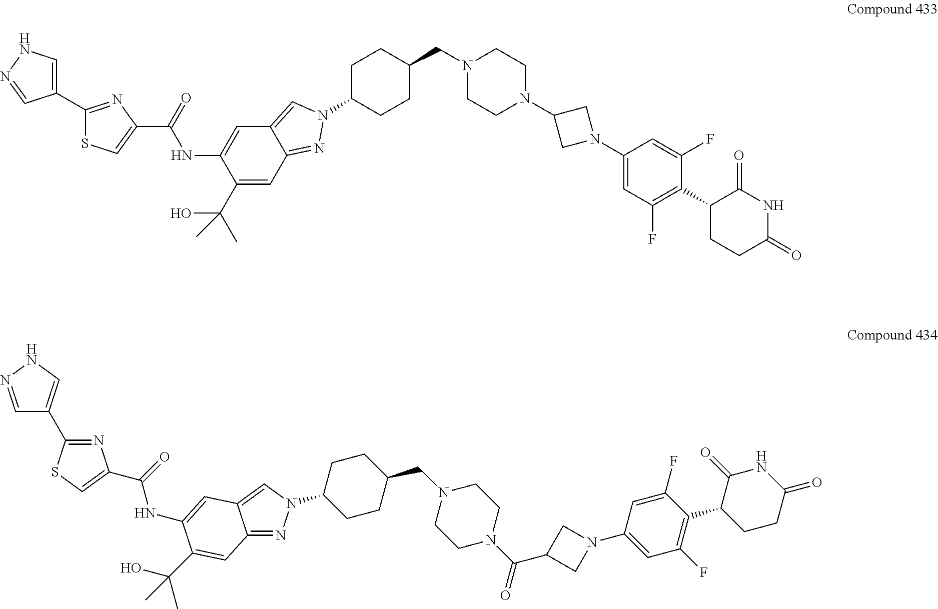 [SMILES: CC(C)(O)c1cc2nn([C@H]3CC[C@H](CN4CCN(C5CN(c6cc(F)c([C@H]7CCC(=O)NC7=O)c(F)c6)C5)CC4)CC3)cc2cc1NC(=O)c1csc(-c2cn[nH]c2)n1 ; CC(C)(O)c1cc2nn([C@H]3CC[C@H](CN4CCN(C(=O)C5CN(c6cc(F)c([C@H]7CCC(=O)NC7=O)c(F)c6)C5)CC4)CC3)cc2cc1NC(=O)c1csc(-c2cn[nH]c2)n1]

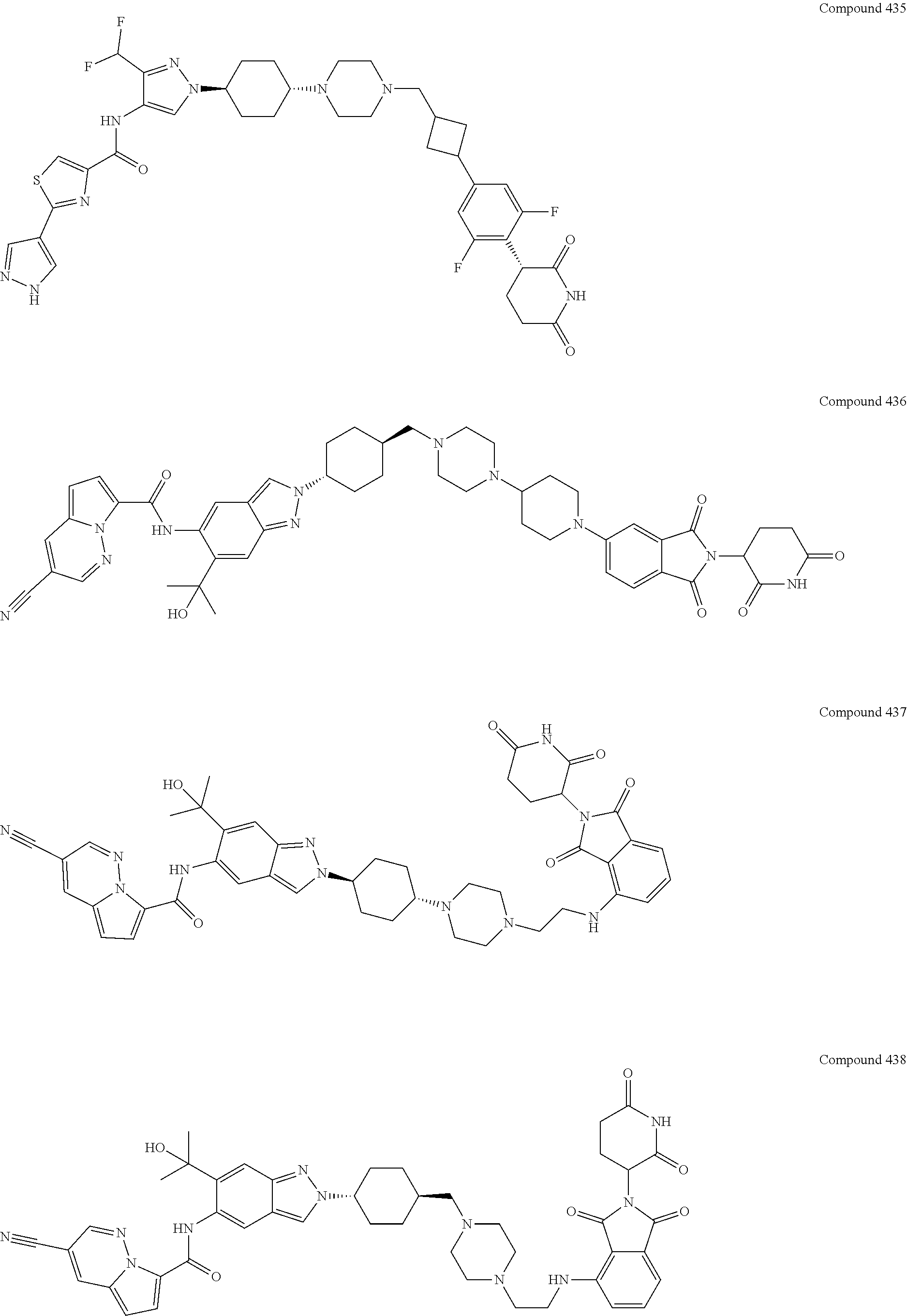 [SMILES: O=C1CC[C@H](c2c(F)cc(C3CC(CN4CCN([C@H]5CC[C@H](n6cc(NC(=O)c7csc(-c8cn[nH]c8)n7)c(C(F)F)n6)CC5)CC4)C3)cc2F)C(=O)N1 ; CC(C)(O)c1cc2nn([C@H]3CC[C@H](CN4CCN(C5CCN(c6ccc7c(c6)C(=O)N(C6CCC(=O)NC6=O)C7=O)CC5)CC4)CC3)cc2cc1NC(=O)c1ccc2cc(C#N)cnn12 ; CC(C)(O)c1cc2nn([C@H]3CC[C@H](N4CCN(CCNc5cccc6c5C(=O)N(C5CCC(=O)NC5=O)C6=O)CC4)CC3)cc2cc1NC(=O)c1ccc2cc(C#N)cnn12 ; CC(C)(O)c1cc2nn([C@H]3CC[C@H](CN4CCN(CCNc5cccc6c5C(=O)N(C5CCC(=O)NC5=O)C6=O)CC4)CC3)cc2cc1NC(=O)c1ccc2cc(C#N)cnn12]

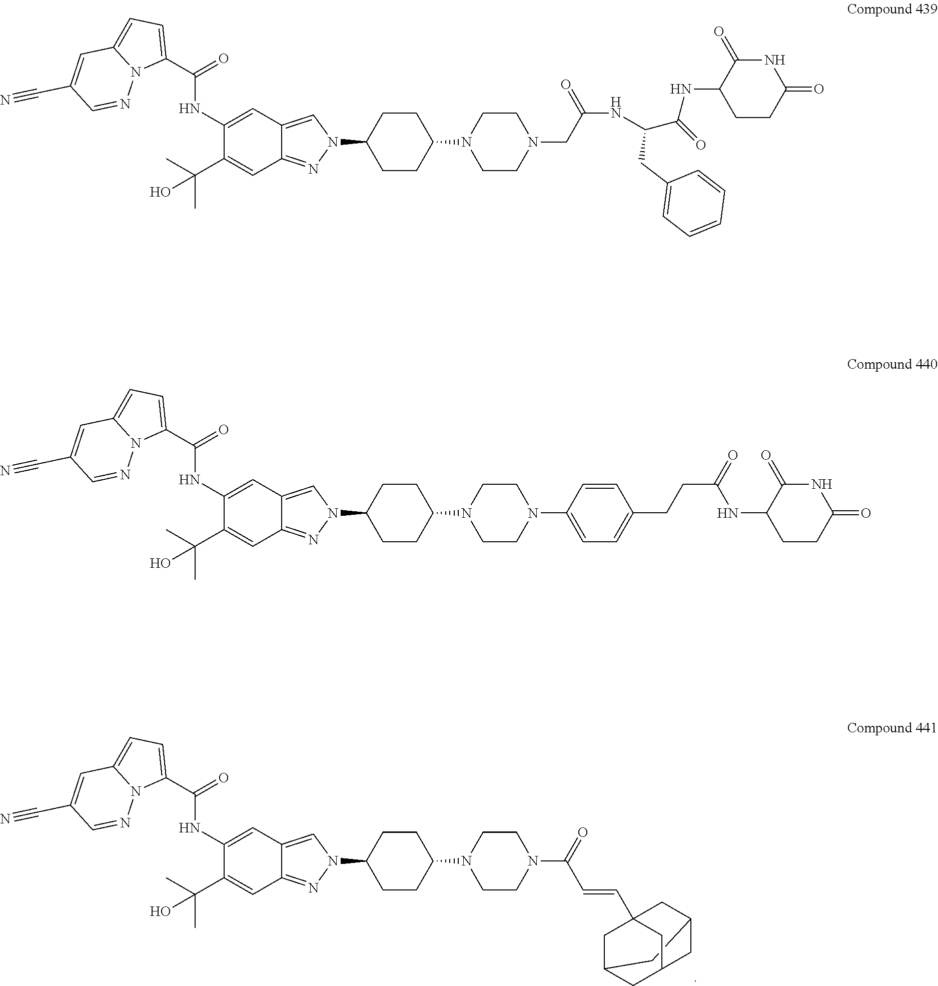 [SMILES: CC(C)(O)c1cc2nn([C@H]3CC[C@H](N4CCN(CC(=O)N[C@@H](Cc5ccccc5)C(=O)NC5CCC(=O)NC5=O)CC4)CC3)cc2cc1NC(=O)c1ccc2cc(C#N)cnn12 ; CC(C)(O)c1cc2nn([C@H]3CC[C@H](N4CCN(c5ccc(CCC(=O)NC6CCC(=O)NC6=O)cc5)CC4)CC3)cc2cc1NC(=O)c1ccc2cc(C#N)cnn12 ; CC(C)(O)c1cc2nn([C@H]3CC[C@H](N4CCN(C(=O)/C=C/C56CC7CC(CC(C7)C5)C6)CC4)CC3)cc2cc1NC(=O)c1ccc2cc(C#N)cnn12]

Aspect 47. A pharmaceutical composition comprising a compound of any one of Aspects 1-45 or a pharmaceutically acceptable salt, stereoisomer, tautomer or prodrug thereof, together with a pharmaceutically acceptable excipient.

Aspect 48. A method of treating a disease that can be affected by IRAK4 modulation, comprises administrating a subject in need thereof an effective amount of a compound of any one of Aspects 1-45 or a pharmaceutically acceptable salt, stereoisomer, tautomer or prodrug thereof.

Aspect 49. The method of Aspect 47, wherein the disease is selected from Autoimmune disease and Inflammatory disorders, preferred Systemic Lupus, Hidradenitis suppurativa, Rheumatoid arthritis, Arthritis, Gout, Multiple sclerosis, Psoriasis and cancer, preferred Acute myeloid leukemia Cancer, Lymphoma, B-cell Myelodysplasia.

Aspect 50. Use of a compound of any one of Aspects 1-45 or a pharmaceutically acceptable salt, stereoisomer, tautomer or prodrug thereof in the preparation of a medicament for treating a disease that can be affected by IRAK4 modulation.

Aspect 51. The use of Aspect 49, wherein the disease is cancer, preferred pancreatic cancer, breast cancer, glioblastoma multiforme, head and neck cancer, or non-small cell lung cancer.

The inventors of the instant invention surprisingly found that the linker like

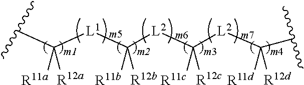

moiety between IRAK4 binder moieties and E3 ligase Ligand moieties imparts increased degradation activity other compounds indicating that the compounds could degrade the target protein (hereby means the IRAK4 protein) with high efficiency. Specifically, the DC₅₀ values of the compounds disclosed herein are lower than 20 nm, preferably lower than 10 nm, more preferably lower than 5 nm, 3 nm, 2 nm or 1 nm; the Dₘₐₓ values are higher than 60%, preferably higher than 70% or 80%.

In the preferred linkers, m1, m2, m3 and m4 are each independently 0, 1 or 2; m5 and m7 are each independently 0 or 1; and m6 is 0, 1 or 2; preferably m1 and m2 are 0; m3 and m4 are each independently 0; For 2; m5 and m7 are each independently 1; and m6 is 1 or 2. More preferably, the

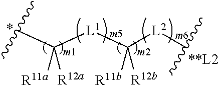

moiety is

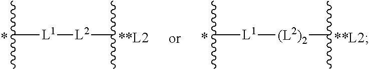

wherein, L¹ is selected from —O—, —N(R^{a})—,

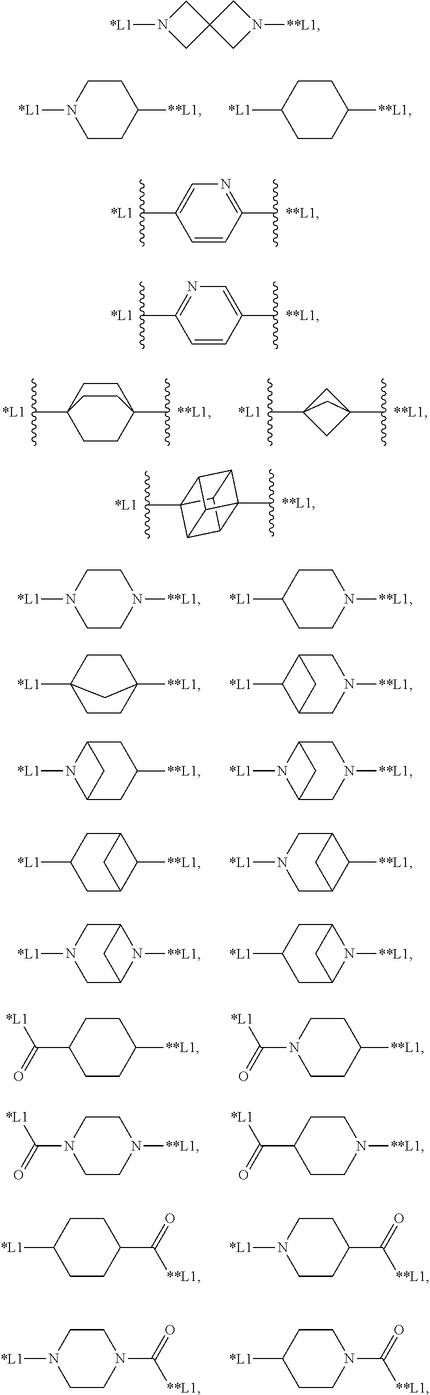

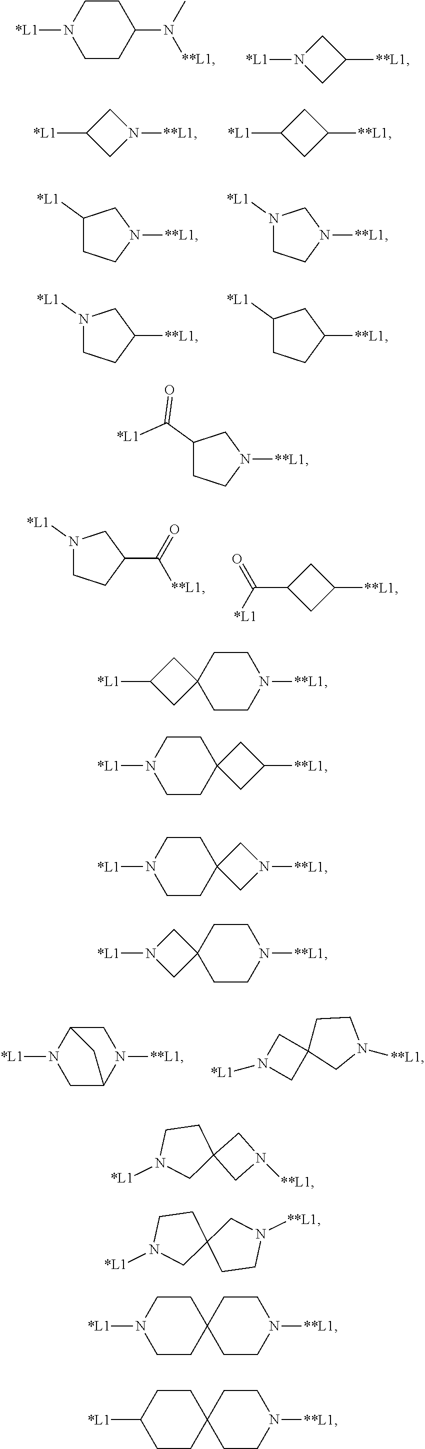

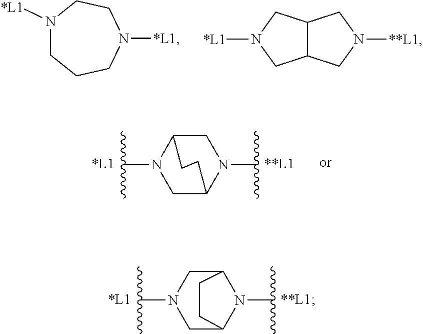

wherein each of said

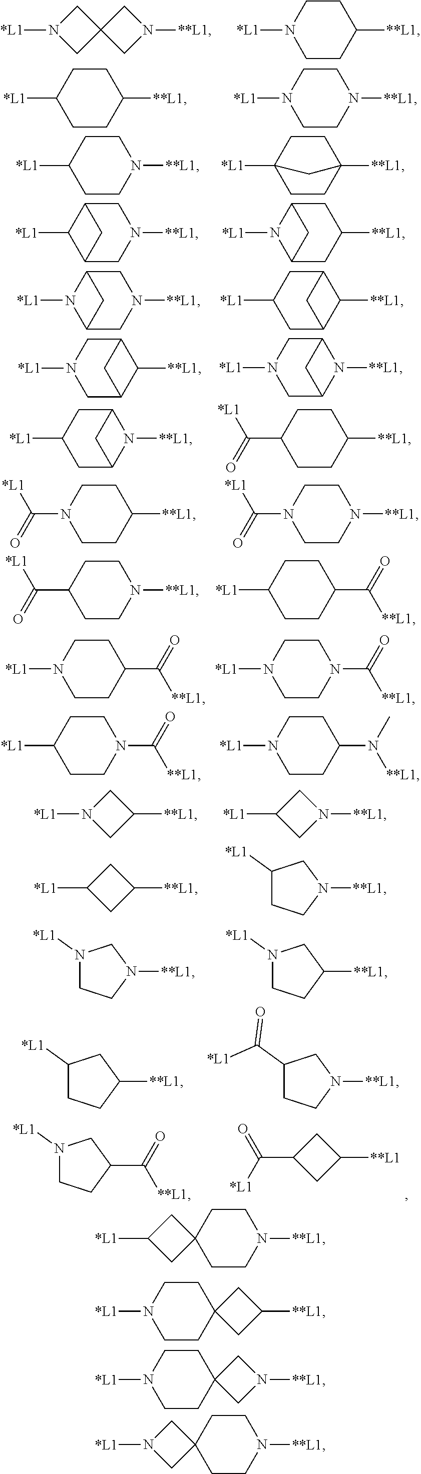

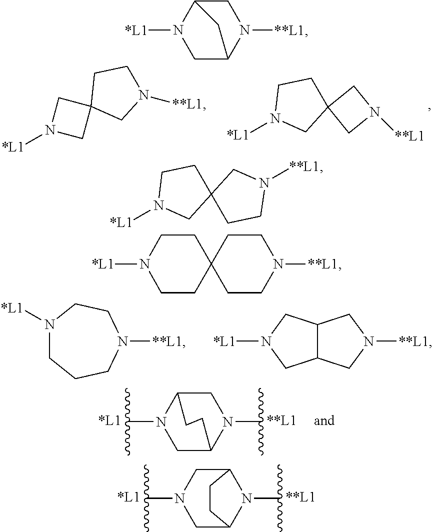

is optionally substituted with at least one R^{L1e};
each of said R^{L1c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L1c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, or octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; preferably, L¹ is selected from —O—, —N(CH₃)—,

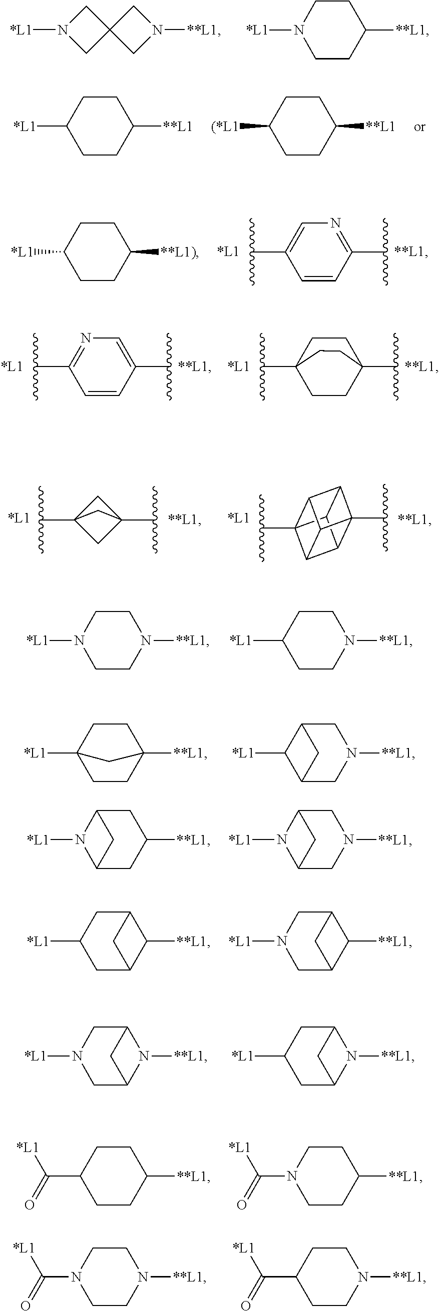

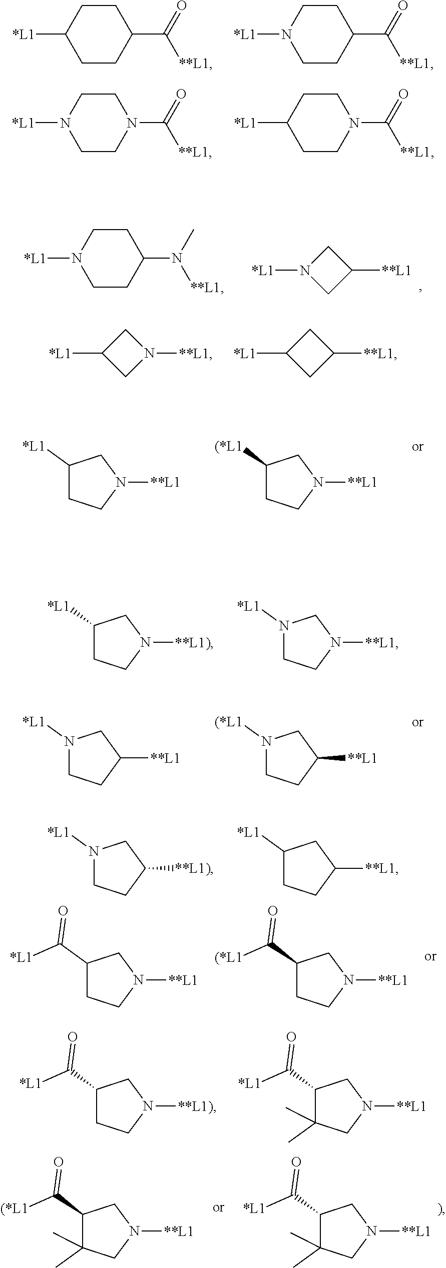

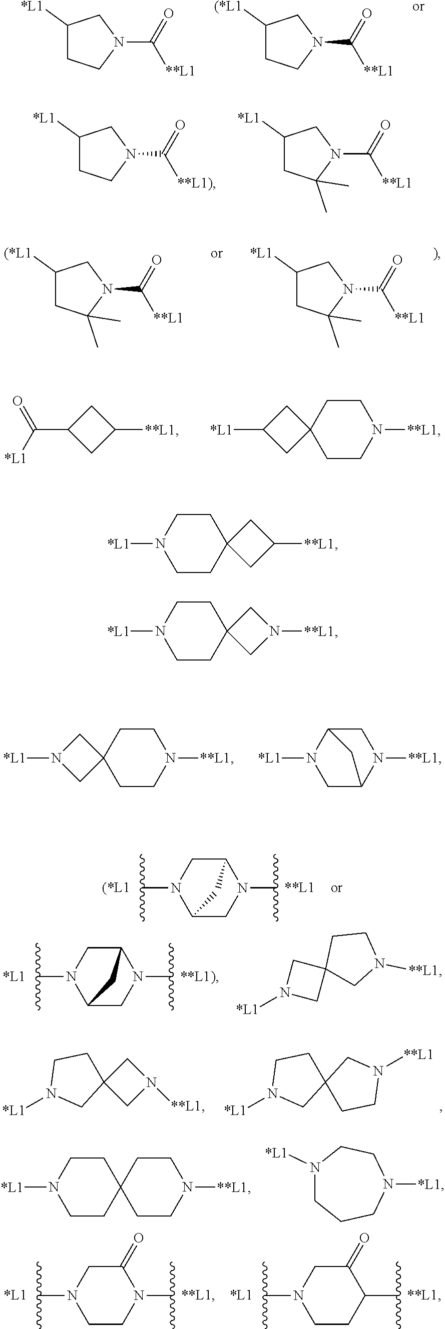

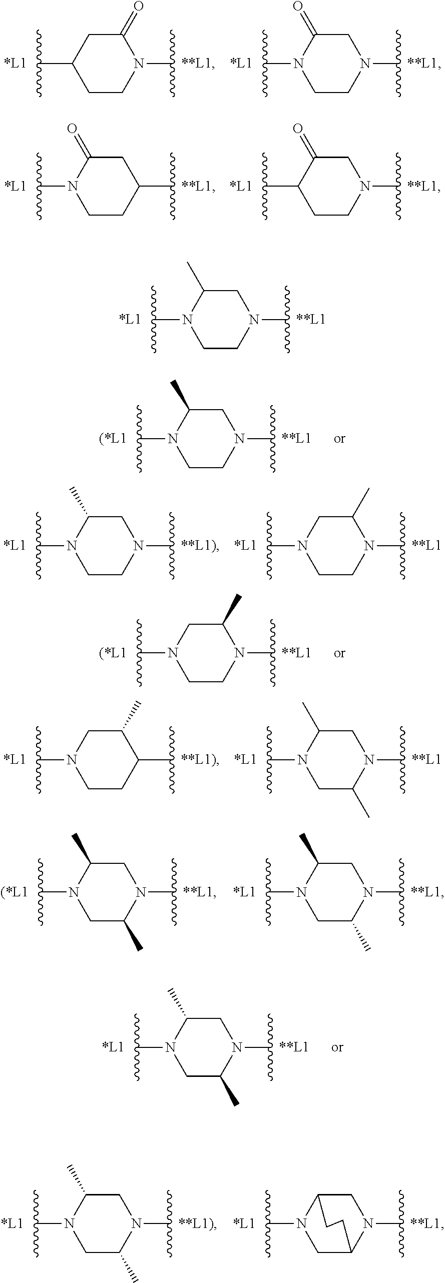

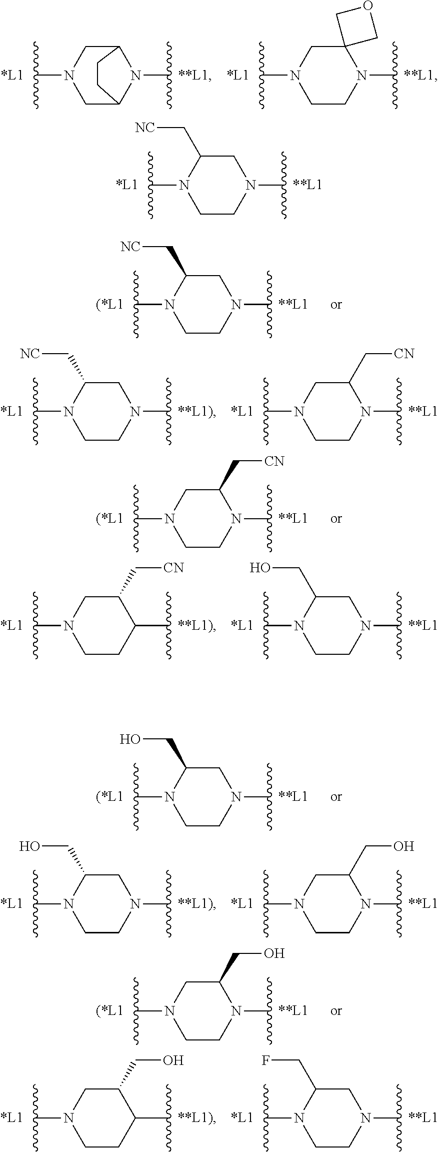

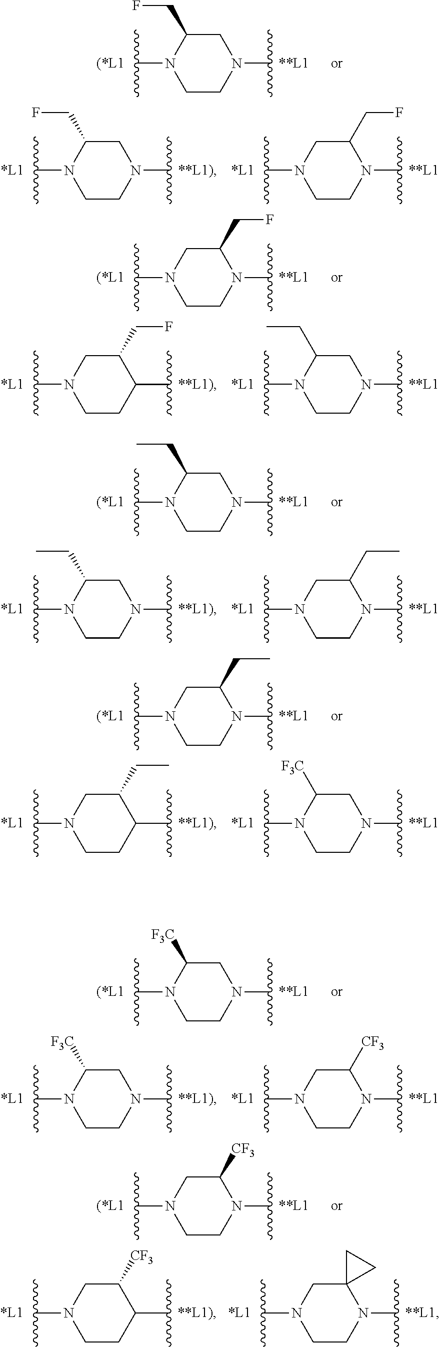

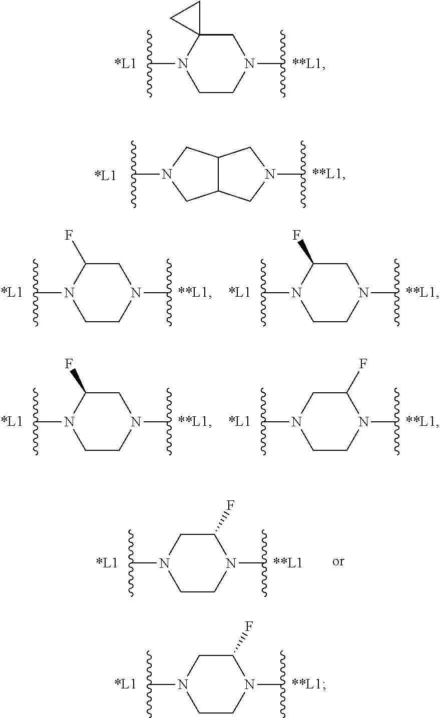

wherein L² is selected from —O—, —N(R^{a})—,

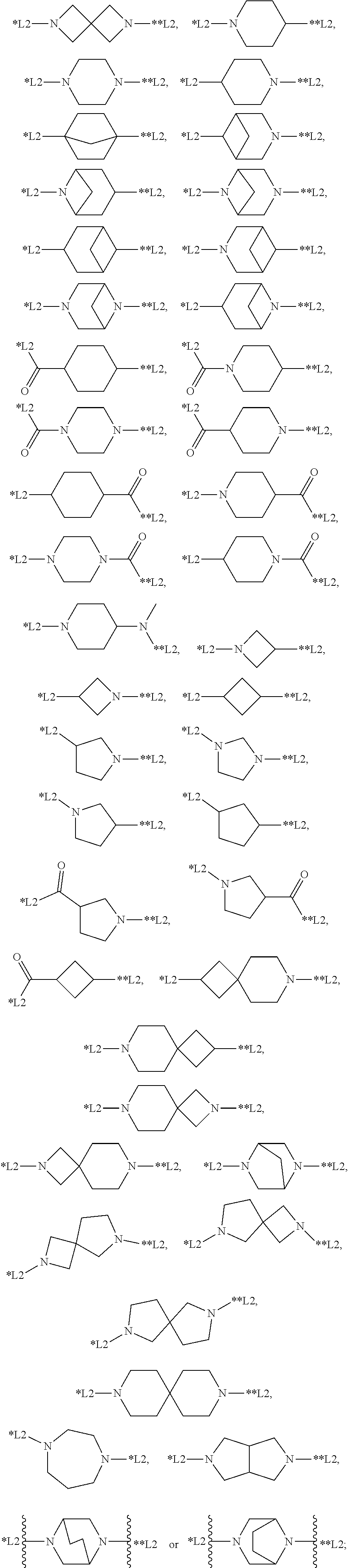

wherein each of said

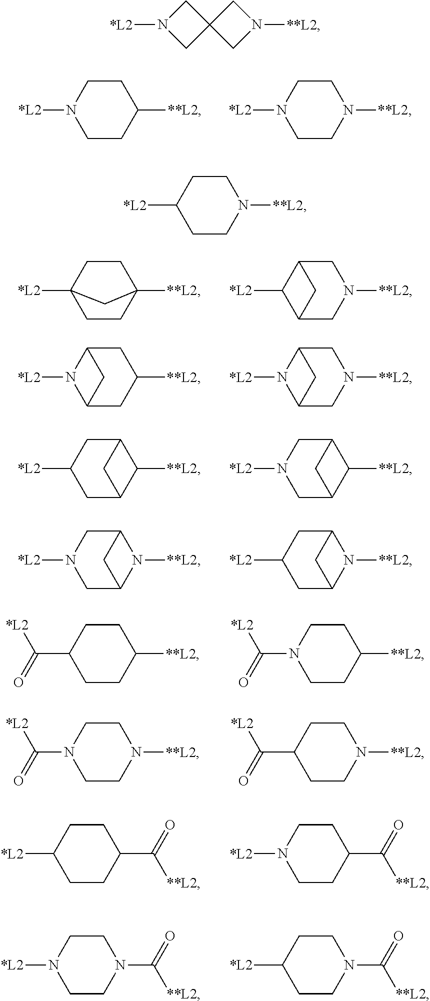

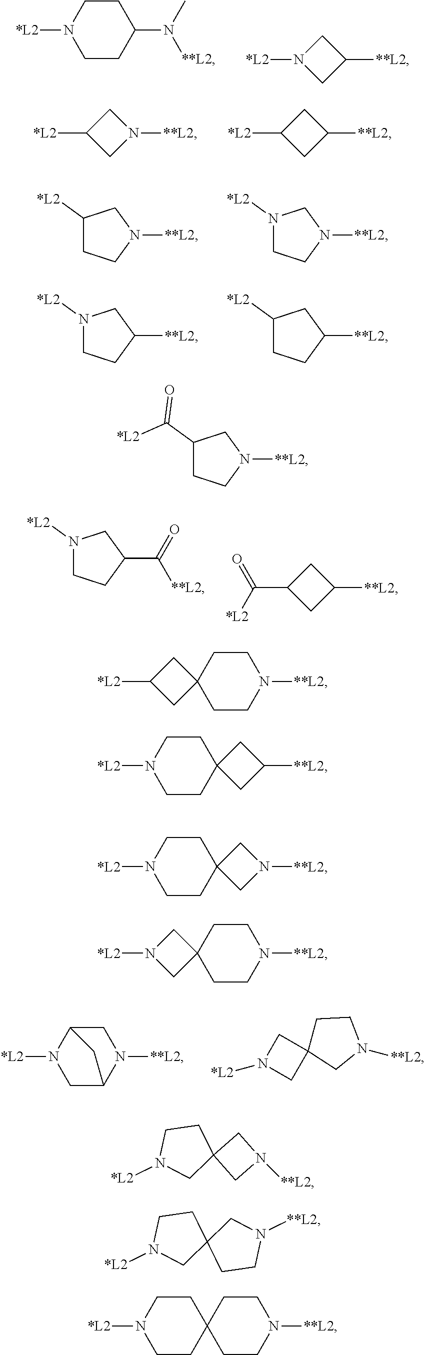

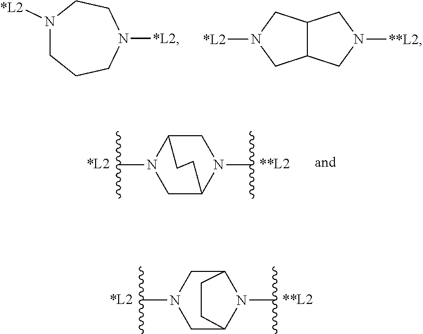

is optionally substituted with at least one R^{L2c};
each of said R^{L2c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca}
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L2c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, propyl, butyl, pentyl, henyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; preferably L² is selected from —O—, —N(CH₃)—,

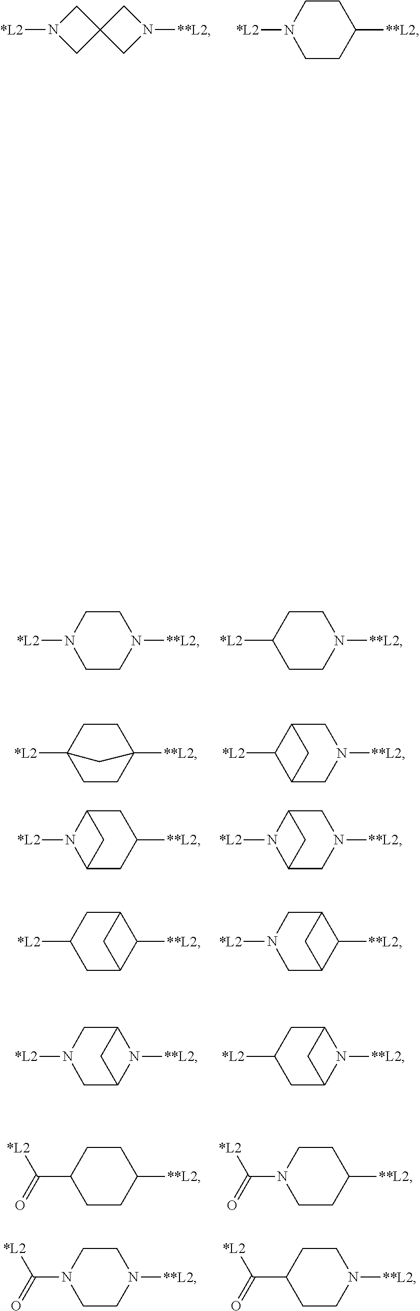

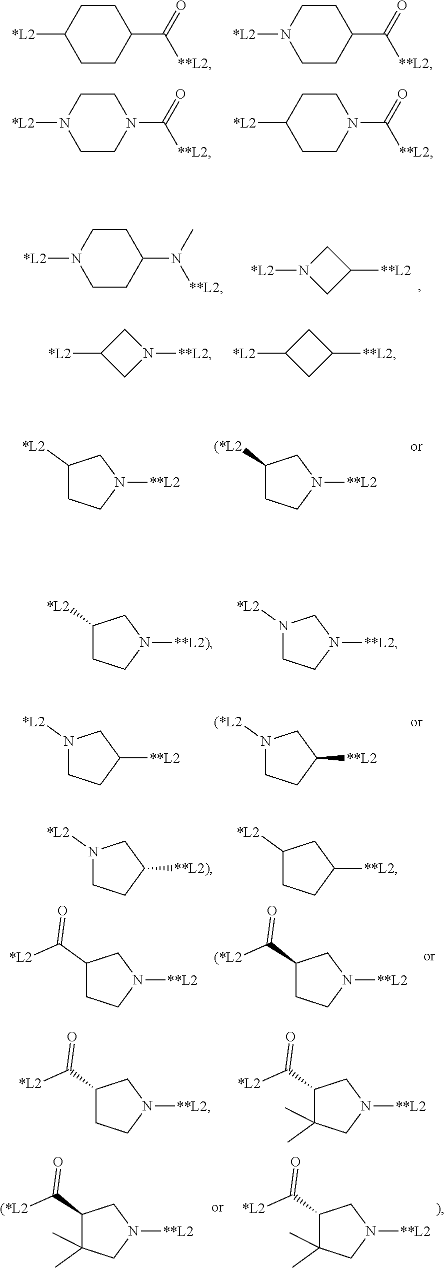

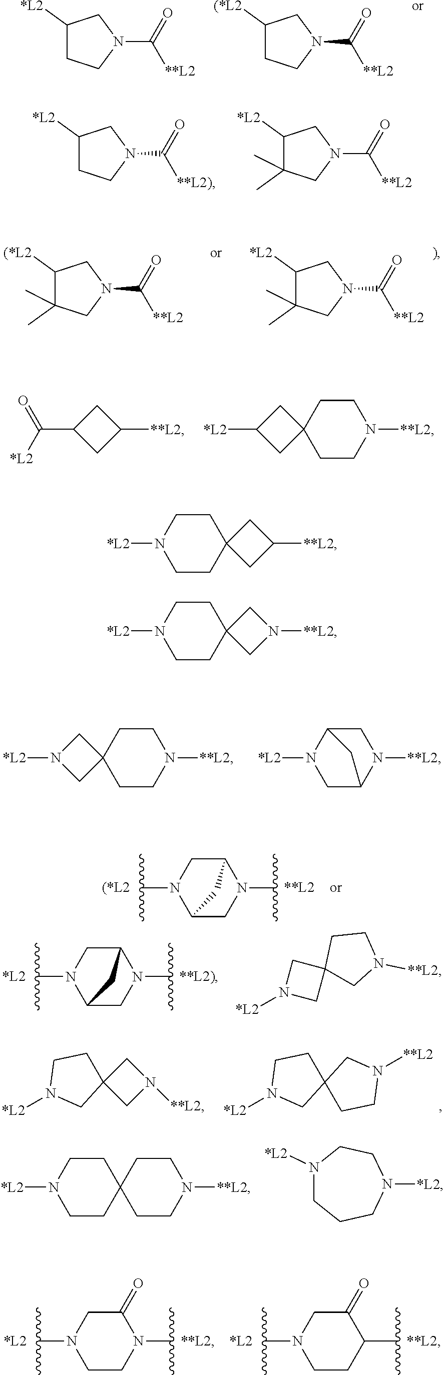

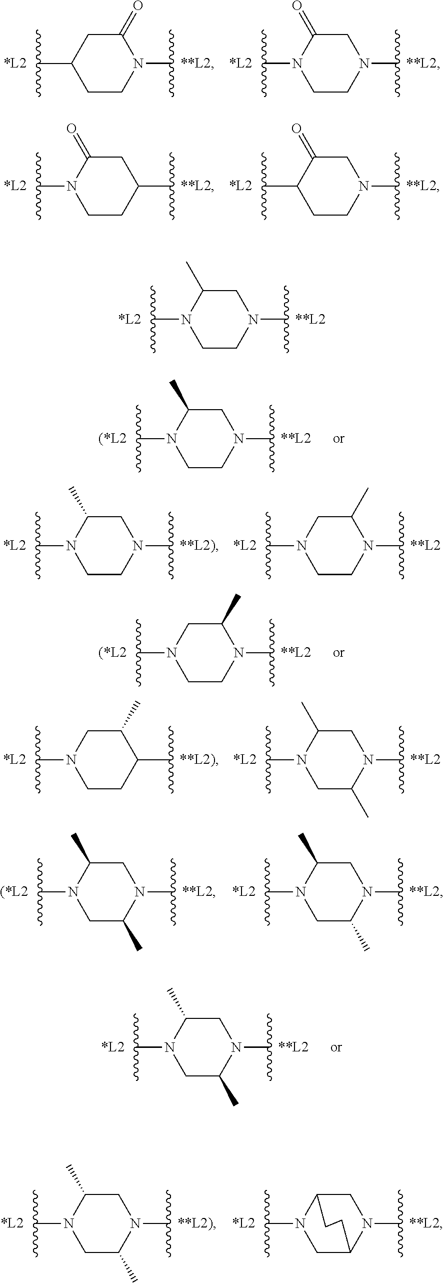

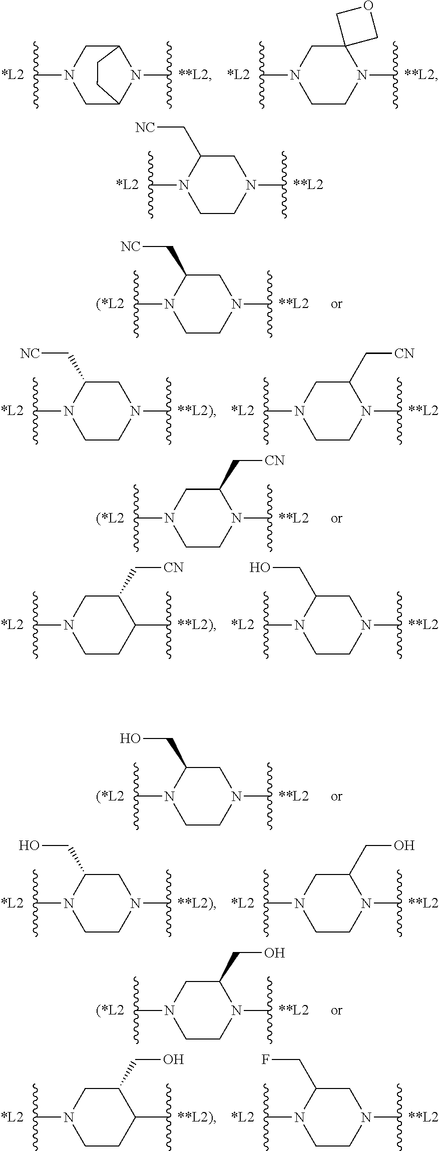

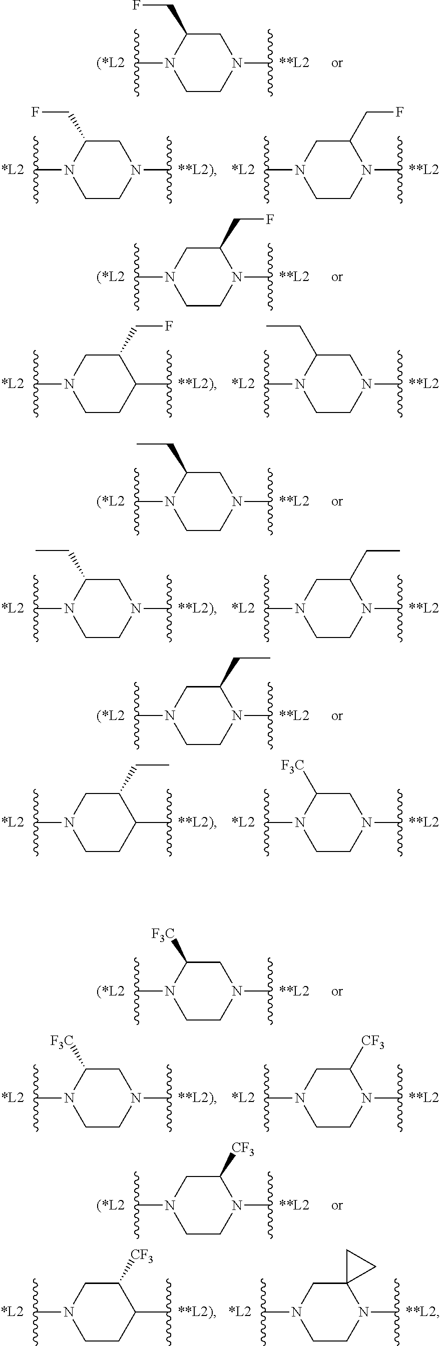

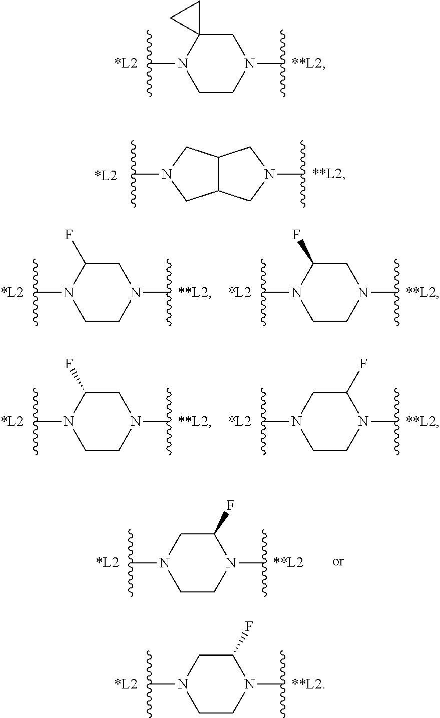

Even more preferably, the

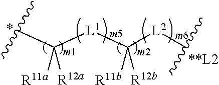

moiety is

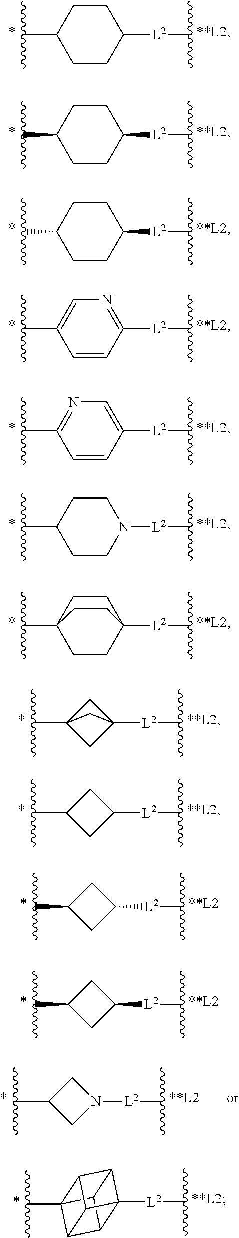

more preferably, the

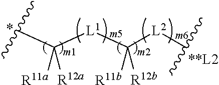

moiety is

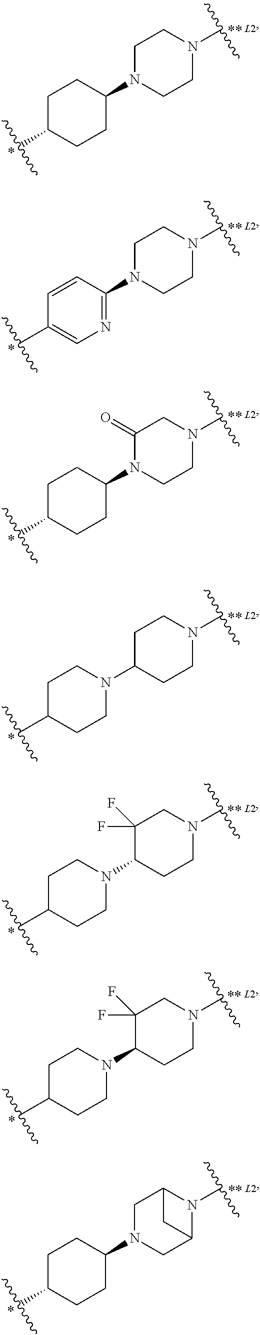

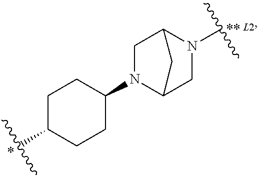 [SMILES: *N1CC2CC1CN2[C@H]1CC[C@H](*)CC1]

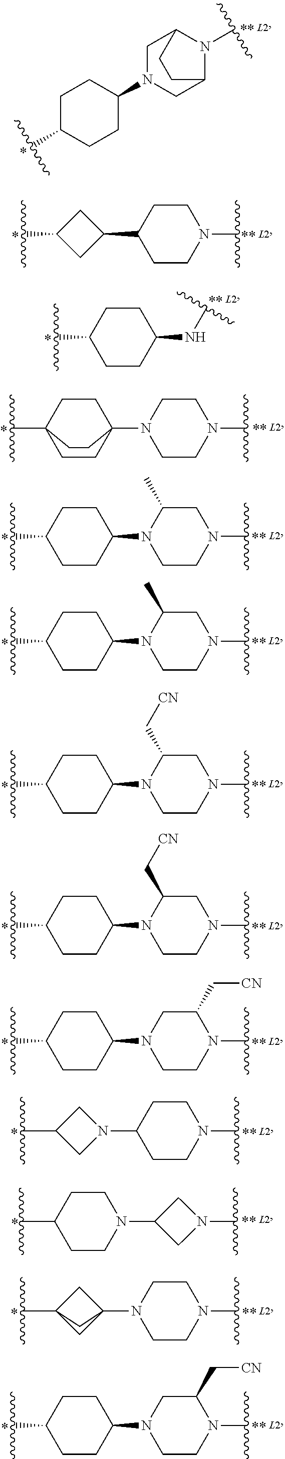

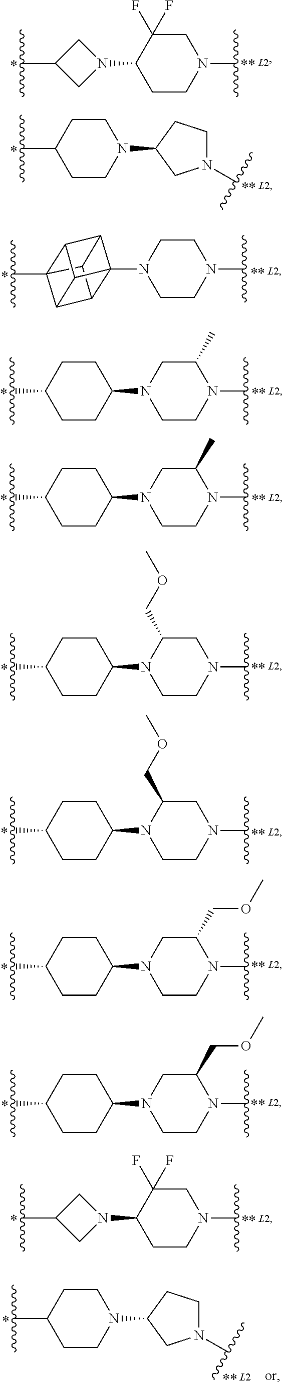

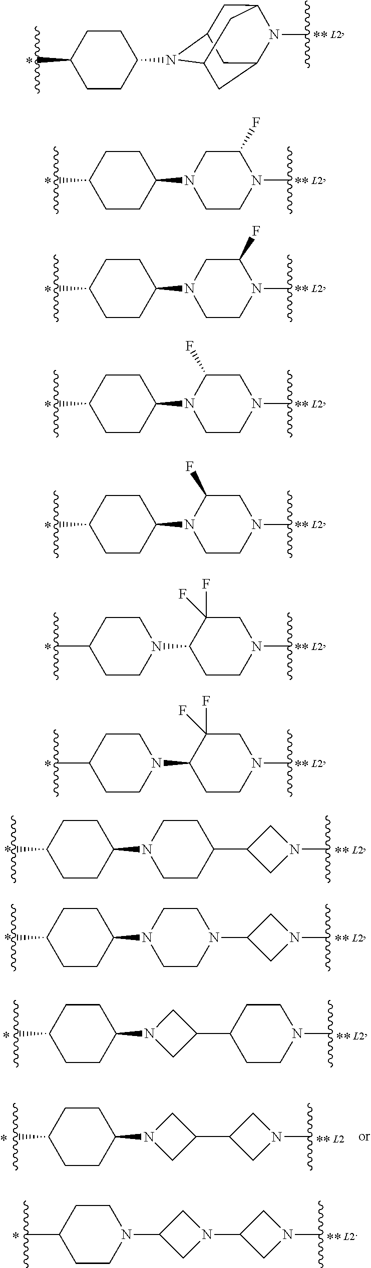

### DETAILED DESCRIPTION OF THE INVENTION

The following terms have the indicated meanings throughout the specification:

As used herein, including the appended Aspects, the singular forms of words such as “a” “an”, and “the” include their corresponding plural references unless the context clearly dictates otherwise.

The term “or” is used to mean, and is used interchangeably with, the term “and/or” unless the context clearly dictates otherwise.

The term “alkyl” refers to a hydrocarbon group selected from linear and branched saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl ort-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “cycloalkyl” refers to a hydrocarbon group selected from saturated cyclic hydrocarbon groups, comprising monocyclic and polycyclic (e.g., bicyclic and tricyclic) groups including fused, bridged or spiro cycloalkyl. In one embodiment, said “cycloalkyl” is anyone of “—C₃-C₈cycloalkyl”, “—C₃-C₁₂cycloalkyl”, “cyclopropyl”, “cyclobutyl”, “cyclopentyl”, “cyclohexyl”, “cycloheptyl” or “cyclooctyl” described in the specification or the claims.

The term “aryl” used alone or in combination with other terms refers to a group selected from: 5- and 6-membered carbocyclic aromatic rings, e.g., phenyl;
bicyclic ring systems such as 7- to 12-membered bicyclic ring systems, wherein at least one ring is carbocyclic and aromatic, e.g., naphthyl and indanyl; and,
tricyclic ring systems such as 10- to 15-membered tricyclic ring systems wherein at least one ring is carbocyclic and aromatic, e.g., fluorenyl.

In one embodiment, said “aryl” is anyone of “C₆-C₁₂aryl” described in the specification or the claims.

The terms “aromatic hydrocarbon ring” and “aryl” are used interchangeable throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

The term “aryl-alkyl-” refers to an alkyl group as defined above which is further substituted by an aryl group. Examples of an aryl-alkyl group include aryl-C₁₋₈alkyl, such as phenylethyl, or phenylmethyl (benzyl).

The term “heteroaryl” refers to a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

In one embodiment, said “heteroaryl” is anyone of “5- to 12-membered heteroaryl”, “5-membered heteroaryl”, “6-membered heteroaryl”, “7-membered heteroaryl”, “8-membered heteroaryl”, “9-membered heteroaryl”, “10-membered heteroaryl”, “11-membered heteroaryl” or “12-membered heteroaryl” described in the specification or the claims.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides. The term “C-linked heteroaryl” as used herein means that the heteroaryl group is connected to the core molecule by a bond from a C-atom of the heteroaryl ring. The terms “aromatic heterocyclic ring” and “heteroaryl” are used interchangeable throughout the disclosure herein. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur (S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur (S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur (S) and oxygen (O). In some embodiments, the monocyclic or bicyclic aromatic heterocyclic ring is an 8- to 10-membered heteroaryl ring, which is bicyclic and which has 1 or 2 heteroatom ring members independently selected from nitrogen, sulfur and oxygen.

“Heterocyclyl”, “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups. The term “optionally oxidized sulfur” used herein refers to S, SO or SO₂.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, the reference to one isomer applies to any of the possible isomers. Whenever the isomeric composition is unspecified, all possible isomers are included. The term “substantially pure” as used herein means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

When compounds disclosed herein contain a di-substituted cyclohexyl or cyclobutyl group, substituents found on cyclohexyl or cyclobutyl ring may adopt cis and trans formations. Cis formation means that both substituents are found on the upper side of the 2 substituent placements on the carbon, while trans would mean that they were on opposing sides.

It may be advantageous to separate reaction products from one another and/or from starting materials. The desired product of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation. “Diastereomers” refers to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column. “Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and/or water and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” includes salts of at least one compound of Formula (I), and salts of the stereoisomers of the compound of Formula (I), such as salts of enantiomers, and/or salts of diastereomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as the contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as a compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined above, a disease or disorder in a subject. In the case of combination therapy, the “therapeutically effective amount” refers to the total amount of the combination objects for the effective treatment of a disease, a disorder or a condition.

The pharmaceutical composition comprising the compound disclosed herein can be administrated via oral, inhalation, rectal, parenteral or topical administration to a subject in need thereof. For oral administration, the pharmaceutical composition may be a regular solid formulation such as tablets, powder, granule, capsules and the like, a liquid formulation such as water or oil suspension or other liquid formulation such as syrup, solution, suspension or the like; for parenteral administration, the pharmaceutical composition may be a solution, water solution, oil suspension concentrate, lyophilized powder or the like. Preferably, the formulation of the pharmaceutical composition is selected from a tablet, coated tablet, capsule, suppository, nasal spray or injection, more preferably tablet or capsule. The pharmaceutical composition can be a single unit administration with an accurate dosage. In addition, the pharmaceutical composition may further comprise additional active ingredients.

All formulations of the pharmaceutical composition disclosed herein can be produced by the conventional methods in the pharmaceutical field. For example, the active ingredient can be mixed with one or more excipients, then to make the desired formulation. The “pharmaceutically acceptable excipient” refers to conventional pharmaceutical carriers suitable for the desired pharmaceutical formulation, for example: a diluent, a vehicle such as water, various organic solvents, etc., a filler such as starch, sucrose, etc. a binder such as cellulose derivatives, alginates, gelatin and polyvinylpyrrolidone (PVP); a wetting agent such as glycerol; a disintegrating agent such as agar, calcium carbonate and sodium bicarbonate; an absorption enhancer such as quaternary ammonium compound; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises other pharmaceutically acceptable excipients such as a decentralized agent, a stabilizer, a thickener, a complexing agent, a buffering agent, a permeation enhancer, a polymer, aromatics, a sweetener, and a dye.

The term “disease” refers to any disease, discomfort, illness, symptoms or indications, and can be interchangeable with the term “disorder” or “condition”.

Throughout this specification and the Aspects which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the Aspects which follow, the term “Cₙ₋ₘ” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

### Abbreviations

| | |
|---|---|
| (Boc)₂O | di-tert-butyl dicarbonate |
| Ac₂O | Acetic anhydride |
| AcOH | Acetic Acid |
| DCM | dichloromethane |
| DIEA | diisopropylethylamine |
| DMAP | 4-dimethylaminopyridine |
| DMF | Dimethylformamide |
| DMSO | Dimethyl sulfoxide |
| EA | Ethyl acetate |
| EA, EtOAc | Ethyl acetate |
| EDC | 1-(3-Dimethylaminopropyl)-3-ethylcarbodiimide |
| | hydrochloride |
| EDCI | N-Ethyl-N′-(3-dimethylaminopropyl)carbodiimide |
| | hydrochloride |
| Eq. | equivalent |
| Et₃N, TEA | triethyl amine |
| EtOH | ethanol |
| Fe | iron |
| HATU | 1-[bis(dimethylamino)methylene]-1H-1,2,3-triazolo[4,5-b] |
| | pyridinium 3-oxid hexafluorophosphate |
| HCl | Hydrochloric Acid |
| HOBT | 1-Hydroxybenzotriazole |
| MeCN | acetonitrile |
| MeOH | Methanol |
| NaBH₄ | Sodium borohydride |
| NBS | N-Bromosuccinimide |
| Pd(dppf)Cl₂ | [1,1′-Bis(diphenylphosphino)ferrocene] |
| | dichloropalladium(II) |
| Pd(OAc)₂ | Palladium(II)acetate |
| PE | petroleum ether |
| PreP-HPLC | Preparative High-Performance Liquid Chromatography |
| Prep-TCL | Preparative thin-layer chromatography |
| rt | room temperature |
| t-BuONO | tert-Butyl nitrite |
| TFA | trifluoroacetic acid |
| THF | tetrahydrofuran |
| TLC | thin-layer chromatography |
| TMSCN | Trimethylsilyl cyanide |
| Tol | toluene |

### Examples

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, Pharmablock, Bidepharm or TCI, and were used without further purification unless indicated otherwise. Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

¹H NMR spectra were recorded on Agilent instruments operating at 400 MHz or 500 MHz. ¹HNMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₃CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LCMS-1: LC-MS spectrometer (Agilent 1260 Infinity) Detector: MWD (190-400 nm), Mass detector: 6120 SQ Mobile phase: A: water with 0.1% Formic acid, B: acetonitrile with 0.1% Formic acid Column: Poroshell 120 EC-C18, 4.6×50 mm 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS, LCMS-3: LC-MS spectrometer (Agilent 1260 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS-2: LC-MS spectrometer (Agilent 1290 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.2 mL/min Time (min) A (%) B (%)

| Time (min) | A(%) | B(%) |
|---|---|---|
| 0.00 | 90 | 10 |
| 1.5 | 5 | 95 |
| 2.0 | 5 | 95 |
| 2.1 | 90 | 10 |
| 3.0 | 90 | 10 |

Preparative HPLC was conducted on a column (150×21.2 mm ID, 5 pm, Gemini NXC 18) at a flow rate of 20 ml/min, injection volume 2 ml, at room temperature and UV Detection at 214 nm and 254 nm.

### Common Intermediate A

### 1-(4-(2,4-dioxo-3-((2-(trimethylsilyl)ethoxy)methyl)tetrahydropyrimidin-1(2H)-yl)phenyl)azetidine-3-carbaldehyde

### Step 1: 4-(2,4-dioxo-3-((2-(trimethylsilyl)ethoxy)methyl)tetrahydropyrimidin-1(2H)-yl)phenyl trifluoromethanesulfonate

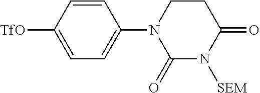 [SMILES: C[Si](C)(C)CCOCN1C(=O)CCN(c2ccc(OS(=O)(=O)C(F)(F)F)cc2)C1=O]

To a solution of 1-(4-hydroxyphenyl)-3-((2-(trimethylsilyl)ethoxy)methyl)dihydropyrimidine-2,4(1H,3H)-dione (obtained through the similar way described in WO2022012622 A1) (500 mg, 1.49 mmol) in DCM, pyridine (235 mg, 2.98 mmol) was added. Then Tf₂O (630 mg, 2.24 mmol) was added dropwise between 0° C. and 5° C. The mixture was allowed to slowly warm up to rt from 0° C. and stirred at room temperature for 3 h. The resulting mixture was concentrated under reduced pressure. The residue was purified with silica gel column chromatography, eluted with PE/EtOAc (100%:0% to 50%:50%) to afford 4-(2,4-dioxo-3-((2-(trimethylsilyl)ethoxy)methyl)tetrahydropyrimidin-1(2H)-yl)phenyl trifluoromethanesulfonate (644 mg, 92%). [M+H]⁺=469.5.

### Step 2: 1-(4-(3-(hydroxymethyl)azetidin-1-yl)phenyl)-3-((2-(trimethylsilyl)ethoxy)methyl)dihydropyrimidine-2,4(1H3H)-dione

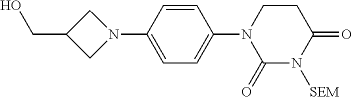 [SMILES: C[Si](C)(C)CCOCN1C(=O)CCN(c2ccc(N3CC(CO)C3)cc2)C1=O]

To a mixture of 4-(2,4-dioxo-3-((2-(trimethylsilyl)ethoxy)methyl)tetrahydropyrimidin-1(2H)-yl)phenyl trifluoromethanesulfonate (100 mg, 0.213 mol), azetidin-3-ylmethanol hydrochloride (39 mg, 0.32 mmol), Pd₂(dba)₃ (20 mg, 0.0213 mmol), Ruphos (20 mg, 0.0426 mmol) and Cs₂CO₃ (138 mg, 0.426 mmol) in dioxane (10 mL) was stirred at 100° C. for overnight under nitrogen. Upon completion monitored with LCMS, the mixture was allowed to cool to room temperature, and concentrated under vacuum. The residue was purified with silica gel column chromatography, eluted with PE/EA (100%:0% to 50%:50%) to afford 1-(4-(3-(hydroxymethyl)azetidin-1-yl)phenyl)-3-((2-(trimethylsilyl)ethoxy)methyl)dihydropyrimidine-2,4(1H,3H)-dione (67 mg, 78%). [M+H]⁺=406.4.

### Step 3: 1-(4-(2,4-dioxo-3-((2-(trimethylsilyl)ethoxy)methyl)tetrahydropyrimidin-1(2H)-yl)phenyl)azetidine-3-carbaldehyde

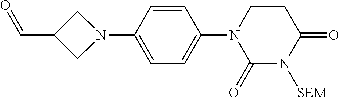 [SMILES: C[Si](C)(C)CCOCN1C(=O)CCN(c2ccc(N3CC(C=O)C3)cc2)C1=O]

To a solution of 1-(4-(3-(hydroxymethyl)azetidin-1-yl)phenyl)-3-((2-(trimethylsilyl)ethoxy)methyl)dihydropyrimidine-2,4(1H,3H)-dione (132 mg, 0.325 mmol) in DMSO (10 mL), IBX (114 mg, 0.406 mmol) was added. The mixture was stirred at room temperature overnight. The resulting mixture was extracted with EA (50 mL*3), washed with brine (100 mL), dried over Na₂SO₄, and concentrated under reduced pressure to afford 1-(4-(2,4-dioxo-3-((2-(trimethylsilyl)ethoxy)methyl)tetrahydropyrimidin-1(2H)-yl)phenyl)azetidine-3-carbaldehyde (140 mg, crude). [M+H]⁺=404.5

### Common Intermediate B

### 1-(4-(2,4-dioxotetrahydropyrimidin-1(2H)-yl)phenyl)azetidine-3-carboxylic acid

### Step 1: tert-butyl 1-(4-(2,4-dioxo-3-((2-(trimethylsilyl)ethoxy)methyl)tetrahydropyrimidin-1(2H)-yl)phenyl)azetidine-3-carboxylate

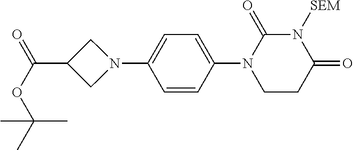 [SMILES: CC(C)(C)OC(=O)C1CN(c2ccc(N3CCC(=O)N(COCC[Si](C)(C)C)C3=O)cc2)C1]

A mixture of 4-(2,4-dioxo-3-((2-(trimethylsilyl)ethoxy)methyl)tetrahydropyrimidin-1(2H)-yl)phenyl trifluoromethanesulfonate (468 mg, 1.0 mol), tert-butyl azetidine-3-carboxylate hydrochloride (188 mg, 1.2 mmol), Pd₂(dba)₃ (92 mg, 0.1 mmol), Ruphos (93.4 mg, 0.2 mmol) and Cs₂CO₃ (652 mg, 2.0 mmol) in dioxane (50 mL) was stirred at 90° C. overnight under nitrogen. Upon completion monitored with LCMS, the mixture was allowed to cool to room temperature, and concentrated under vacuum. The residue was purified with silica gel column chromatography, eluted with PE/EA (100%:0% to 50%:50%) to afford the product (460 mg, 97%). [M+H]⁺=476.2.

### Step 2: 1-(4-(2,4-dioxotetrahydropyrimidin-1(2H)-yl)phenyl)azetidine-3-carboxylicacid

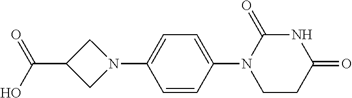 [SMILES: O=C1CCN(c2ccc(N3CC(C(=O)O)C3)cc2)C(=O)N1]

To a solution of tert-butyl 1-(4-(2,4-dioxo-3-((2-(trimethylsilyl)ethoxy)methyl)tetrahydropyrimidin-1(2H)-yl)phenyl)azetidine-3-carboxylate (460 mg, 0.97 mmol) in Dichloromethane (10 mL), TFA (10 mL) was added. The mixture was stirred at room temperature for overnight. The reaction mixture was concentrated under vacuum. The residue was diluted with MeOH (20 mL) and NH₃·H₂O (0.5 mL) was added dropwise. The mixture was stirred at room temperature for 0.5 h, then concentrated under vacuum to afford desired product (260 mg, 93%). [M+H]⁺=290.1

### Common Intermediate C

### tert-butyl 4-((trans)-4-(4-amino-3-carbamoyl-1H-pyrazol-1-yl)cyclohexyl)piperazine-1-carboxylate

### Step 1: (cis)-4-(4-benzylpiperazin-1-yl)cyclohexan-1-ol

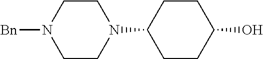 [SMILES: O[C@H]1CC[C@@H](N2CCN(Cc3ccccc3)CC2)CC1]

To a solution of (cis)-4-aminocyclohexan-1-ol hydrochloride (100 g, 660 mmol) and benzyl bis(2-chloroethyl)amine hydrochloride (212.6 g, 791 mmol) in EtOH (2 L) was added sodium carbonate (224.2 g, 2.64 mol) at 0° C. After stirring at 90° C. for 5 hours, the mixture was filtered and concentrated to yield the crude product, which was purified with silica gel column chromatography (dichloromethane:methanol=10:1) to afford the compound (82.6 g, 43%). ¹H NMR (400 MHz, DMSO-d6) δ 7.33-7.21 (m, 5H), 4.30-4.20 (m, 1H), 3.68 (s, 1H), 3.42 (s, 2H), 2.45-2.35 (m, 7H), 2.15-2.10 (m, 1H), 1.66-1.58 (m, 4H), 1.40-1.33 (m, 4H). [M+H]⁺=275.2.

### Step 2: (cis)-4-(piperazin-1-yl)cyclohexan-1-ol (Intermediate 3)

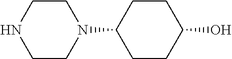 [SMILES: O[C@H]1CC[C@@H](N2CCNCC2)CC1]

To a solution of (cis)-4-(4-benzylpiperazin-1-yl)cyclohexan-1-ol (82.6 g, 301 mmol) in EtOH (2 L) was added 10% palladium on activated carbon (9.61 g) at room temperature. After stirring at 40° C. under 15 psi hydrogen atmosphere overnight, the mixture was filtered and concentrated to yield the product (53.4 g, 92%). ¹H NMR (400 MHz, CDCl₃) δ 3.97-3.95 (m, 1H), 2.91-2.88 (m, 4H), 2.56-2.54 (m, 4H), 2.27-2.21 (m, 1H), 1.85-1.50 (m, 8H). [M+H]⁺=185.1

### Step 3: (cis)-tert-butyl 4-(4-hydroxycyclohexyl)piperazine-1-carboxylate

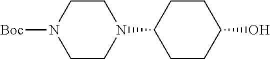 [SMILES: CC(C)(C)OC(=O)N1CCN([C@H]2CC[C@@H](O)CC2)CC1]

To a solution of (cis)-4-(piperazin-1-yl)cyclohexan-1-ol (53.4 g, 290 mmol) in THF (600 mL) was added saturated sodium bicarbonate aqueous solution (300 mL) and di-tert-butyl decarbonate (94.9 g, 435 mmol) at 0° C. After stirring at 0° C. for 2 hours, the mixture was diluted with water (500 mL) and extracted with ethyl acetate (700 mL×2). The combined organic layer was washed with brine (600 mL), dried over Na₂SO₄, filtered and concentrated to yield an oil, which was dripped into petroleum ether until a large amount of solid precipitated. The solids were filtered and filter cake was washed with petroleum ether (100 mL), filtratered and dried in oven at 40° C. for 0.5 hour to yield the product (66 g, 76%). ¹H NMR (400 MHz, CDCl₃) δ 4.00-3.93 (m, 1H), 3.44-3.41 (m, 4H), 2.52-2.50 (m, 4H), 2.32-2.25 (m, 1H), 1.85-1.59 (m, 8H), 1.46 (s, 9H). [M+H]⁺=285.2.

### Step 4: (cis)-tert-butyl 4-[4-(methanesulfonyloxy)cyclohexyl]piperazine-1-carboxylate

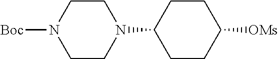 [SMILES: CC(C)(C)OC(=O)N1CCN([C@H]2CC[C@@H](OS(C)(=O)=O)CC2)CC1]

To a solution of (cis)-tert-butyl 4-(4-hydroxycyclohexyl)piperazine-1-carboxylate (66.0 g, 232 mmol) and triethylamine (47.0 g, 464 mmol) in dichloromethane (1 L) was added methanesulfonyl chloride (31.9 g, 279 mmol) at 0° C. The mixture was stirred at 0° C. for 2 hours, and water (1 L) was added. The mixture was extracted with dichloromethane (300 mL×3). The combined organic layer was washed with water (500 mL), brine (500 mL), dried over Na₂SO₄, filtered and concentrated under reduced pressure to afford the product (73 g, 78%). ¹H NMR (400 MHz, CDCl₃) δ 4.97 (br s, 1H), 3.44-3.41 (m, 4H), 3.01 (s, 3H), 2.52-249 (m, 4H), 2.34-2.29 (m, 1H), 2.16-2.13 (m, 2H), 1.70-1.64 (m, 6H), 1.46 (s, 9H). [M+H]⁺=363.2.

### Step 5: tert-butyl 4-((trans)-4-(3-carbamoyl-4-nitro-1H-pyrazol-1-yl)cyclohexyl)piperazine-1-carboxylate

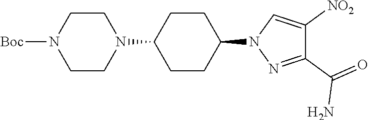 [SMILES: CC(C)(C)OC(=O)N1CCN([C@H]2CC[C@H](n3cc([N+](=O)[O-])c(C(N)=O)n3)CC2)CC1]

To a solution of 4-nitro-1H-pyrazole-3-carboxamide (7.0 g, 44.8 mmol) and (cis)-tert-butyl 4-[4-(methanesulfonyloxy)cyclohexyl]piperazine-1-carboxylate (17.9 g, 49.3 mmol) in DMF (300 mL) was added cesium carbonate (29.2 g, 89.6 mmol) at room temperature. After stirring at 130° C. for 16 hours, the mixture was diluted with water (600 mL) and extracted with ethyl acetate (500 mL×2). The combined organic layer was washed with brine (500 mL), dried over Na₂SO₄, filtered and concentrated under reduced pressure to afford the crude compound, which was purified with C18 column (acetonitrile:water=50% to 75%) to yield the product (2 g, 11%). [M+H]⁺=423.2.

### Step 6: tert-butyl 4-((trans)-4-(4-amino-3-carbamoyl-1H-pyrazol-1-yl)cyclohexyl)piperazine-1-carboxylate

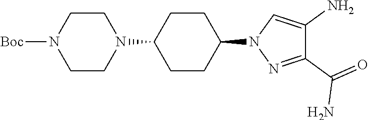 [SMILES: CC(C)(C)OC(=O)N1CCN([C@H]2CC[C@H](n3cc(N)c(C(N)=O)n3)CC2)CC1]

To a THE solution (200 mL) of tert-butyl 4-((trans)-4-(3-carbamoyl-4-nitro-1H-pyrazol-1-yl)cyclohexyl)piperazine-1-carboxylate (5.9 g, 14 mmol) was added 10% palladium on activated carbon (450 mg) at room temperature. After stirring at 25° C. under 15 psi hydrogen atmosphere for 5 hours, the mixture was filtered and concentrated to yield the title compound (5.1 g, 88%). ¹H NMR (400 MHz, CDCl₃) δ 6.95 (s, 1H), 6.56 (br s, 1H), 5.26 (br s, 1H), 4.14 (br s, 2H), 3.96-3.88 (m, 1H), 3.45-3.42 (m, 4H), 2.54-2.51 (m, 4H), 2.43-2.34 (m, 1H), 2.19 (d, J=12 Hz, 2H), 2.03 (d, J=12.4 Hz, 2H), 1.80-1.71 (m, 2H), 1.46-1.40 (m, 11H). [M+H]⁺=393.2.

### Common Intermediate D

### tert-butyl 4-((trans)-4-(4-amino-3-(difluoromethyl)-1H-pyrazol-1-yl)cyclohexyl)piperazine-1-carboxylate

### Step 1: 1-benzyl-1H-pyrazole-3-carbaldehyde

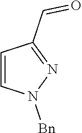 [SMILES: O=Cc1ccn(Cc2ccccc2)n1]

To a DMF solution (500 mL) of 1H-pyrazole-3-carbaldehyde (50.0 g, 0.52 mol) and benzyl bromide (93.5 g, 0.546 mol) was added cesium carbonate (423.9 g, 1.30 mol). After stirring at 25° C. for 1 hour, the mixture was added to water (2.0 L), extracted with ethyl acetate (300 mL×2). The combined organic layer was washed with brine (2.0 L), dried with Na₂SO₄, filtered and concentrated under reduced pressure to afford the crude compound, which was purified with silica gel column chromatography (PE:EtOAc=20:1) to yield the product (65 g, 64%). ¹H NMR (300 MHz, CDCl₃) δ 10.00 (s, 1H), 7.43-7.37 (m, 4H), 7.32-7.27 (m, 2H), 6.84 (s, 1H), 5.41 (s, 2H). [M+H]⁺=187.0.

### Step 2: 1-benzyl-3-(difluoromethyl)-1H-pyrazole

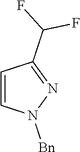 [SMILES: FC(F)c1ccn(Cc2ccccc2)n1]

To a dichloromethane solution (500 mL) of 1-benzyl-1H-pyrazole-3-carbaldehyde (65 g, 0.335 mol) was added diethylaminosulfur trifluoride (216.1 g, 1.34 mol) at 0° C. After stirring at 20° C. for 16 hours, the mixture was added iced water (1.0 L), extracted with dichloromethane (300 mL×2). The combined organic layer was washed with brine (1.0 L), dried with Na₂SO₄, filtered and concentrated under reduced pressure to afford the crude compound, which was purified with silica gel column chromatography (PE:EtOAc=40:1) to yield the product (48.5 g, 70%). ¹H NMR (300 MHz, CDCl₃) δ 7.38-7.34 (m, 4H), 7.26-7.21 (m, 2H), 6.89-6.50 (m, 2H), 5.32 (s, 2H). [M+H]⁺=209.1

### Step 3: 3-(difluoromethyl)-1H-pyrazole

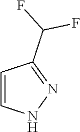

To a MeOH solution (500 mL) of 1-benzyl-3-(difluoromethyl)-1H-pyrazole (25 g, 0.120 mol) was added 10% wt. Pd(OH)₂ on carbon (2.5 g), 10% wt. Pd on carbon (2.5 g), and 12 M hydrochloric acid (13 mL). After stirred under H₂ (50 psi) at 40° C. for 16 hours, the mixture was filtered and concentrated under reduced pressure to yield the product (14 g, 99%). ¹H NMR (300 MHz, CDCl₃) δ 7.88 (s, 1H), 7.02 (t, J=54.9 Hz, 1H), 6.55 (s, 1H). [M+H]⁺=119.0

### Step 4: 3-(difluoromethyl)-4-nitro-1H-pyrazole

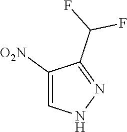 [SMILES: O=[N+]([O-])c1c[nH]nc1C(F)F]

To a concentrated sulfuric acid solution (150 mL) of 3-(difluoromethyl)-1H-pyrazole (14 g, 0.119 mol) was added concentrated nitric acid (28.7 g, 0.297 mol) at 0° C. The mixture was stirred at 115° C. for 4 hours, then added to iced water (500 g), extracted with ethyl acetate (250 mL×2). The combined organic layer was washed with brine (300 mL), dried with Na₂SO₄, filtered and concentrated under reduced pressure to afford the crude compound, which was purified with silica gel column chromatography (PE:EtOAc=3:1) to yield the product (15 g, 78%). [M−H]⁺=161.9.

### Step 5: tert-butyl 4-((trans)-4-(3-(difluoromethyl)-4-nitro-1H-pyrazol-1-yl)cyclohexyl)piperazine-1-carboxylate

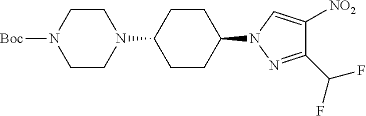 [SMILES: CC(C)(C)OC(=O)N1CCN([C@H]2CC[C@H](n3cc([N+](=O)[O-])c(C(F)F)n3)CC2)CC1]

To a N,N-dimethylformamide solution (600 mL) of 3-(difluoromethyl)-4-nitro-1H-pyrazole (13.0 g, 79.7 mmol) and (cis)-tert-butyl 4-[4-(methanesulfonyloxy)cyclohexyl]piperazine-1-carboxylate (28.9 g, 79.7 mmol) was added cesium carbonate (51.9 g, 0.159 mol). After stirring at 130° C. for 16 hours, the mixture was filtered and diluted with water (600 mL). The mixture extracted with ethyl acetate (100 mL×2). The combined organic layer was washed with brine (500 mL×2), dried over Na₂SO₄, filtered and concentrated under reduced pressure to afford the crude compound, which was purified with silica gel column chromatography (CH₂Cl₂:EtOAc=4:1) to yield the product (6.0 g, 17%). [M+H]⁺=430.2.

### Step 6: tert-butyl 4-((trans)-4-(4-amino-3-(difluoromethyl)-1H-pyrazol-1-yl)cyclohexyl)piperazine-1-carboxylate

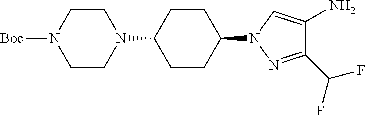 [SMILES: CC(C)(C)OC(=O)N1CCN([C@H]2CC[C@H](n3cc(N)c(C(F)F)n3)CC2)CC1]

To a THE solution (120 mL) of tert-butyl 4-((trans)-4-(3-(difluoromethyl)-4-nitro-1H-pyrazol-1-yl)cyclohexyl)piperazine-1-carboxylate (6.0 g, 13.7 mmol) was added 10% palladium on activated carbon (1.2 g) at room temperature. After stirring at room temperature under 15 psi hydrogen atmosphere for 16 hours, the mixture was filtered and concentrated to yield the crude product, which was recrystallized from ACN (10 mL) at room temperature to afford the product (4.99 g, 88%). ¹H NMR (400 MHz, CDCl₃) δ 7.00 (s, 1H), 6.67 (t, J=54.8 Hz, 1H), 3.96-3.89 (m, 1H), 3.44-3.42 (m, 4H), 3.19 (br s, 2H), 2.52 (br s, 4H), 2.42-2.36 (m, 1H), 2.18 (d, J=12 Hz, 2H), 2.03 (d, J=12.4 Hz, 2H), 1.78-1.67 (m, 2H), 1.46 (s, 9H), 1.43-1.36 (m, 2H). [M+H]⁺=400.2.

### Common intermediate E: (R)-3-(2,6-difluoro-4-(3-oxoazetidin-1-yl)phenyl)piperidine-2,6-dione

### Step 1: 2,6-bis(benzyloxy)-3-(4-(3-(benzyloxy)azetidin-1-yl)-2,6-difluorophenyl)pyridine

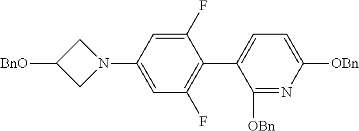 [SMILES: Fc1cc(N2CC(OCc3ccccc3)C2)cc(F)c1-c1ccc(OCc2ccccc2)nc1OCc1ccccc1]

To a solution of 2,6-bis(benzyloxy)-3-(4-bromo-2,6-difluorophenyl)pyridine (20 g, 41.49 mmol), 3-(benzyloxy)azetidine hydrochloride (9.96 g, 49.79 mmol), Pd₂(dba)₃ (3.79 g, 4.15 mmol), RuPhos (3.88 g, 8.3 mmol) and Cs₂CO₃ (40.58 g, 124.47 mmol) in dioxane (400 mL). The mixture was stirred at 100° C. for 3 h under nitrogen atmosphere. After cooled to room temperature, the reaction was quenched with water and extracted with EtOAc. The combined organic layers were washed with brine, dried over anhydrous Na₂SO₄. After filtration, the filtrate was concentrated under reduced pressure. The residue was purified by silica gel column chromatography, eluted with PE/EA (10:1) to afford 2,6-bis(benzyloxy)-3-(4-(3-(benzyloxy)azetidin-1-yl)-2,6-difluorophenyl)pyridine (17 g, 72.6%). [M+H]⁺=565.3.

### Step 2: 3-(2,6-difluoro-4-(3-hydroxyazetidin-1-yl)phenyl)piperidine-2,6-dione

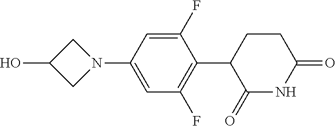 [SMILES: O=C1CCC(c2c(F)cc(N3CC(O)C3)cc2F)C(=O)N1]

To a solution of 2,6-bis(benzyloxy)-3-(4-(3-(benzyloxy)azetidin-1-yl)-2,6-difluorophenyl)pyridine (17 g, 30.09 mmol) in DMF/i-PrOH=(200 mL/600 mL), were added Pd/C (10% wet, 17 g). The mixture was stirred for 2 days at rt ° C. under hydrogen atmosphere. The resulting mixture was filtered, the filter cake was washed with IPA. The filtrate was concentrated under reduced pressure and purified by trituration with DCM/MeOH (10/1) to afford crude 3-(2,6-difluoro-4-(3-hydroxycyclobutyl)phenyl)piperidine-2,6-dione (8 g, 89.9%). [M+H]⁺=297.27.

### Step 3: (R)-3-(2,6-difluoro-4-(3-hydroxyazetidin-1-yl)phenyl)piperidine-2,6-dione

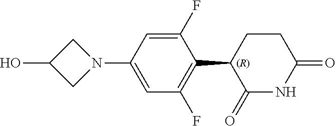

The crude 3-(2,6-difluoro-4-(3-hydroxyazetidin-1-yl)phenyl)piperidine-2,6-dione (76 g) was purified by chiral separation with the following conditions (CHIRALPAK IF (10 cm×25 cm, 10 um), MtBE(0.1% DEA):(MeOH:DCM=1:1)=50:50, 250 mL/min) to afford (R)-3-(2,6-difluoro-4-(3-hydroxyazetidin-1-yl)phenyl)piperidine-2,6-dione (35.83 g, 47.14%, ee=99.54%). [M+H]⁺=297.27

### Step 4: (R)-3-(2,6-difluoro-4-(3-oxoazetidin-1-yl)phenyl)piperidine-2,6-dione

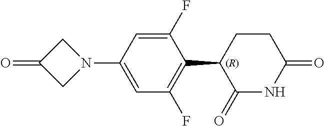

To a solution of (R)-3-(2,6-difluoro-4-(3-hydroxyazetidin-1-yl)phenyl)piperidine-2,6-dione (106 g, 357.78 mmol) in DCM (2 L) was added Dess-martin reagent (455.24 g, 1073.34 mmol) at 0° C. under nitrogen. The mixture was stirred at 0° C. for 3 hours. Then the reaction was diluted with DCM (3 L) and filtered. The filtered cake was washed with DCM (300 mL) and collected. The filtrate was washed with saturated sodium bicarbonate aqueous solution (2 L×2) and brine (2 L×2). The organic layer was dried over anhydrous sodium sulfate and filtered. The solution was concentrated in vacuo. The residue was combined with collected filter cake and dried at reduce pressure to afford (R)-3-(2,6-difluoro-4-(3-oxoazetidin-1-yl)phenyl)piperidine-2,6-dione (91 g, 86.43%). [M+H]⁺=295.27.

### Example 1: N-(3-(difluoromethyl)-1-((1R,4r)-4-((4-(4-((R)-2,6-dioxopiperidin-3-yl)-3,5-difluorophenethyl)piperazin-1-yl)methyl)cyclohexyl)-1H-pyrazol-4-yl)-2-(2-((2,2,2-trifluoroethyl)amino)pyridin-4-yl)oxazole-4-carboxamide

### Step 1: tert-butyl (4-bromopyridin-2-yl)(2,2,2-trifluoroethyl)carbamate

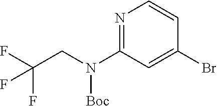 [SMILES: CC(C)(C)OC(=O)N(CC(F)(F)F)c1cc(Br)ccn1]

To a DMF solution (100 mL) of tert-butyl (4-bromopyridin-2-yl)carbamate (9 g, 33.3 mmol) was added 2,2,2-trifluoroethyl trifluoromethanesulfonate (9 g, 39.96 mmol) and Cs₂CO₃ (22 g, 66.6 mmol). The mixture was stirred overnight at room temperature. The solvent was removed under vacuum, and water (100 mL) was added to the residue. The mixture was extracted with EA (100 mL×2). The organic layer was concentrated and purified with combi flash, eluting with EA:PE=0:1˜1:0 to afford the product (9.6 g, 81%). [M+H]⁺=355.2.

### Step 2: ethyl 2-(2-((tert-butoxycarbonyl)(2,2,2-trifluoroethyl)amino)pyridin-4-yl)oxazole-4-carboxylate

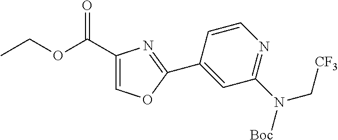 [SMILES: CCOC(=O)c1coc(-c2ccnc(N(CC(F)(F)F)C(=O)OC(C)(C)C)c2)n1]

To a DMF solution (20 mL) of tert-butyl (4-bromopyridin-2-yl)(2,2,2-trifluoroethyl)carbamate (960 mg, 2.7 mmol) was added ethyl oxazole-4-carboxylate (457 mg, 3.24 mmol), Pd(OAc)₂ (61 mg, 0.27 mmol), P(o-tol)₃ (164 mg, 0.54 mmol) and Cs₂CO₃ (1.8 g, 5.4 mmol). The mixture was stirred for 16 h at 80° C. under N₂. The solvent was removed under vacuum, then water (30 mL) was added and extracted with EA (30 mL×3). The organic layer was concentrated and by combi flash, eluting with EA:PE=0:1˜1:0 to afford the product (600 mg, 54%). [M+H]⁺=416.4.

### Step 3: 2-(2-((tert-butoxycarbonyl)(2,2,2-trifluoroethyl)amino)pyridin-4-yl)oxazole-4-carboxylic acid

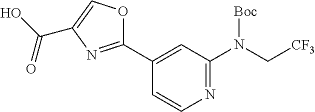 [SMILES: CC(C)(C)OC(=O)N(CC(F)(F)F)c1cc(-c2nc(C(=O)O)co2)ccn1]

Ethyl 2-(2-((tert-butoxycarbonyl)(2,2,2-trifluoroethyl)amino)pyridin-4-yl)oxazole-4-carboxylate (600 mg, 1.45 mmol) and LiOH·H₂O (122 mg, 2.90 mmol) was added to THF (30 mL) and H₂O (10 mL). The mixture was stirred overnight at room temperature. The solvent was removed under vacuum, then water (10 mL) was added and acidified to pH=4 with HCl aq. The solid was filtered and dried to yield the product (410 mg, 73%). [M+H]⁺=388.3.

### Step 4: tert-butyl (4-(4-((3-(difluoromethyl)-1-((1r,4r)-4-(hydroxymethyl)cyclohexyl)-1H-pyrazol-4-yl)carbamoyl)oxazol-2-yl)pyridin-2-yl)(2,2,2-trifluoroethyl)carbamate

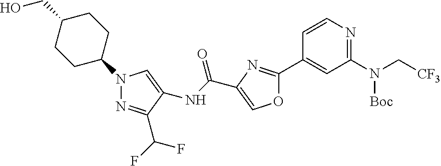 [SMILES: CC(C)(C)OC(=O)N(CC(F)(F)F)c1cc(-c2nc(C(=O)Nc3cn([C@H]4CC[C@H](CO)CC4)nc3C(F)F)co2)ccn1]

To a DMF solution (20 mL) of ((1r,4r)-4-(4-amino-3-(difluoromethyl)-1H-pyrazol-1-yl)cyclohexyl)methanol (639 mg, 2.61 mmol) was added 2-(2-((tert-butoxycarbonyl)(2,2,2-trifluoroethyl)amino)pyridin-4-yl)oxazole-4-carboxylic acid (1200 mg, 3.13 mmol), HATU (1.2 g, 3.13 mmol) and DIEA (708 mg, 5.22 mmol). The mixture was stirred overnight at room temperature under N₂. The solvent was removed under vaccum, then water (50 mL) was added and extracted with EA (50 mL×3). The organic layer was concentrated and purified with combi flash, eluting with EA:PE=0:1˜1:0 to give the product (700 mg, 44%). [M+H]⁺=615.4.

### Step 5: tert-butyl (4-(4-((3-(difluoromethyl)-1-((1r,4r)-4-formylcyclohexyl)-1H-pyrazol-4-yl)carbamoyl)oxazol-2-yl)pyridin-2-yl)(2,2,2-trifloroethyl)carbamate

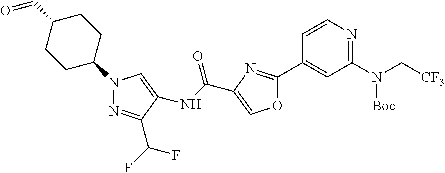 [SMILES: CC(C)(C)OC(=O)N(CC(F)(F)F)c1cc(-c2nc(C(=O)Nc3cn([C@H]4CC[C@H](C=O)CC4)nc3C(F)F)co2)ccn1]

To a DMSO solution (30 mL) of tert-butyl (4-(4-((3-(difluoromethyl)-1-((1r,4r)-4-(hydroxymethyl)cyclohexyl)-1H-pyrazol-4-yl)carbamoyl)oxazol-2-yl)pyridin-2-yl)(2,2,2-trifluoroethyl)carbamate (200 mg, 0.4 mmol) was added IBX (224 mg, 0.8 mmol). The mixture was stirred overnight at room temperature under N₂. Water (100 mL) was added and extracted with EA (30 mL×2). The organic layer was concentrated and purified with combi flash, eluting with EA:PE=0:1˜1:0 to afford the product (140 mg, 29%). [M+H]⁺=613.4.

### Step 6: tert-butyl 4-(((1r,4r)-4-(4-(2-(2-((tert-butoxycarbonyl)(2,2,2-trifluoroethyl)amino)pyridin-4-yl)oxazole-4-carboxamido)-3-(difluoromethyl)-1H-pyrazol-1-yl)cyclohexyl)methyl)piperazine-1-carboxylate

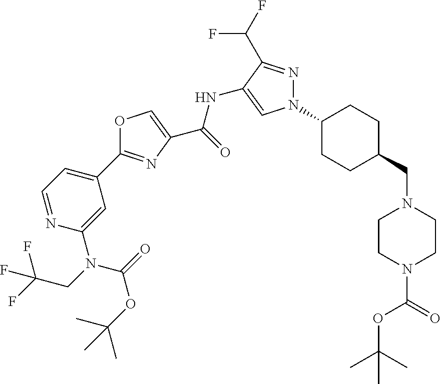 [SMILES: CC(C)(C)OC(=O)N1CCN(C[C@H]2CC[C@H](n3cc(NC(=O)c4coc(-c5ccnc(N(CC(F)(F)F)C(=O)OC(C)(C)C)c5)n4)c(C(F)F)n3)CC2)CC1]

To a DCM solution (20 mL) of tert-butyl (4-(4-((3-(difluoromethyl)-1-((1r,4r)-4-formylcyclohexyl)-1H-pyrazol-4-yl)carbamoyl)oxazol-2-yl)pyridin-2-yl)(2,2,2-trifluoroethyl)carbamate (140 mg, 0.28 mmol) was added tert-butyl piperazine-1-carboxylate (63 mg, 0.336 mmol). The mixture was stirred overnight at room temperature under N₂. To the mixture was added NaBH(Ac)₃ (119 mg, 0.56 mmol), then stirred for 3 h at room temperature under N₂. The reaction was quenched with Na₂CO₃ aq. (15 mL) and extracted with DCM (30 mL×2). The organic layer was dried over with Na₂SO₄, filtered and concentrated to afford the product (200 mg, 91%). [M+H]⁺=783.4.

### Step 7: N-(3-(difluoromethyl)-1-((1r,4r)-4-(piperazin-1-ylmethyl)cyclohexyl)-1H-pyrazol-4-yl)-2-(2-((2,2,2-trifluoroethyl)amino)pyridin-4-yl)oxazole-4-carboxamide

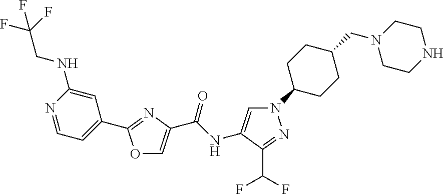 [SMILES: O=C(Nc1cn([C@H]2CC[C@H](CN3CCNCC3)CC2)nc1C(F)F)c1coc(-c2ccnc(NCC(F)(F)F)c2)n1]

To a DCM solution (10 mL) of tert-butyl 4-(((1r,4r)-4-(4-(2-(2-((tert-butoxycarbonyl)(2,2,2-trifluoroethyl)amino)pyridin-4-yl)oxazole-4-carboxamido)-3-(difluoromethyl)-1H-pyrazol-1-yl)cyclohexyl)methyl)piperazine-1-carboxylate (200 mg, 0.26 mmol) was added HCl in 1,4-dioxane (4N, 6 mL). The mixture was stirred overnight at room temperature under N₂. The solvent was removed under vacuum and the residue was diluted with saturated K₂CO₃ aqueous solution, extracted with DCM (3×20 mL), the organic layer was dried over Na₂SO₄, filtered and concentrated to yield the product (148 mg, 100%). [M+H]⁺=583.4.

### Step 8: N-(3-(difluoromethyl)-1-((1R,4r)-4-((4-(4-((R)-2,6-dioxopiperidin-3-yl)-3,5-difluorophenethyl)piperazin-1-yl)methyl)cyclohexyl)-1H-pyrazol-4-yl)-2-(2-((2,2,2-trifluoroethyl)amino)pyridin-4-yl)oxazole-4-carboxamide

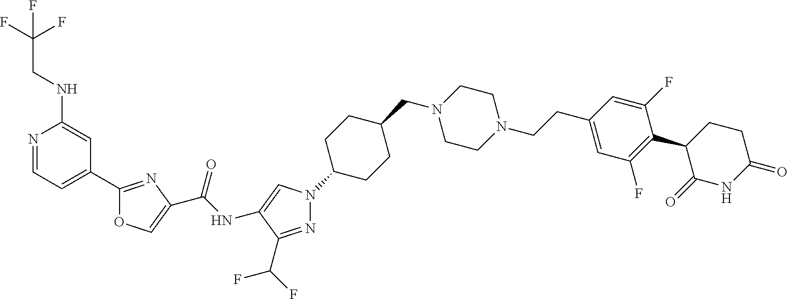 [SMILES: O=C1CC[C@H](c2c(F)cc(CCN3CCN(C[C@H]4CC[C@H](n5cc(NC(=O)c6coc(-c7ccnc(NCC(F)(F)F)c7)n6)c(C(F)F)n5)CC4)CC3)cc2F)C(=O)N1]

To a DCE solution (10 mL) of N-(3-carbamoyl-1-((1r,4r)-4-(piperazin-1-ylmethyl)cyclohexyl)-1H-pyrazol-4-yl)-2-(2-((2,2,2-trifluoroethyl)amino)pyridin-4-yl)oxazole-4-carboxamide (50 mg, 0.086 mmol) and (R)-2-(4-(2,6-dioxopiperidin-3-yl)-3,5-difluorophenyl)acetaldehyde (27 mg, 0.1029 mmol) was added NaBH(OAc)₃ (37 mg, 0.173 mmol), The mixture was stirred for 2 h at 70° C. under N₂. The mixture was purified with combi flash, eluting with DCM:MeOH=1-0 to 1:10 to yield the crude product which was further purified with preparative HPLC to afford the product (14 mg, 19.5%).

¹H NMR (500 MHz, DMSO) δ 10.94 (s, 1H), 9.70 (s, 1H), 8.95 (s, 1H), 8.24 (d, J=5 Hz, 1H), 8.18 (s, 1H), 7.62-7.59 (m, 1H), 7.27-7.05 (m, 3H), 7.02 (d, J=10.0 Hz, 2H), 4.28-4.17 (m, 4H), 2.81-2.73 (m, 2H), 2.54-2.51 (m, 5H), 2.44-2.24 (m, 5H), 2.16-1.98 (m, 6H), 1.98-1.88 (m, 2H), 1.79-1.72 (m, 2H), 1.62-1.56 (m, 1H), 1.07-1.00 (m, 2H). [M+H]⁺=834.6.

### Example 2: N-(3-(difluoromethyl)-1-((1r,4r)-4-((4-(1-(4-(2,4-dioxotetrahydropyrimidin-1(2H)-yl)phenyl)piperidin-4-yl)piperazin-1-yl)methyl)cyclohexyl)-1H-pyrazol-4-yl)-2-(2-((2,2,2-trifluoroethyl)amino)pyridin-4-yl)oxazole-4-carboxamide

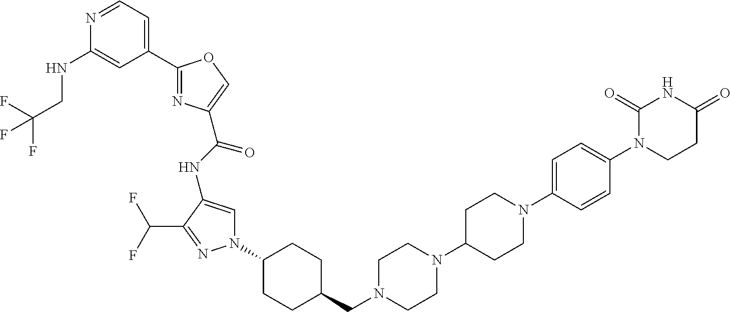 [SMILES: O=C1CCN(c2ccc(N3CCC(N4CCN(C[C@H]5CC[C@H](n6cc(NC(=O)c7coc(-c8ccnc(NCC(F)(F)F)c8)n7)c(C(F)F)n6)CC5)CC4)CC3)cc2)C(=O)N1]

The title compound (6.0 mg, 27.3% yield) was prepared in a manner similar to that described in Example 1. ¹H NMR (500 MHz, DMSO) δ 10.21 (s, 1H), 9.67 (s, 1H), 8.89 (s, 1H), 8.18 (s, 1H), 8.11 (s, 1H), 7.57-7.54 (m, 1H), 7.17 (s, 1H), 7.13 (d, J=10.0 Hz, 1H), 7.09-6.98 (m, 3H), 6.86 (d, J=10.0 Hz, 2H), 4.19-4.14 (m, 3H), 3.65-3.61 (m, 5H), 2.62-2.56 (m, 5H), 2.54-2.51 (m, 2H), 2.30-2.22 (m, 5H), 2.04-1.96 (m, 4H), 1.84-1.67 (m, 6H), 1.51-1.41 (m, 3H), 1.01-0.95 (m, 2H). [M+H]⁺=854.6.

### Example 3: N-(3-(difluoromethyl)-1-((1r,4r)-4-((4-(1-(4-(2,4-dioxotetrahydropyrimidin-1(2H)-yl)phenyl)azetidin-3-yl)piperazin-1-yl)methyl)cyclohexyl)-1H-pyrazol-4-yl)-2-(2-((2,2,2-trifluoroethyl)amino)pyridin-4-yl)oxazole-4-carboxamide

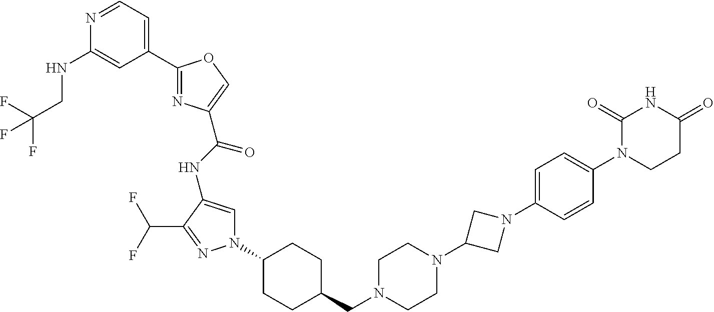 [SMILES: O=C1CCN(c2ccc(N3CC(N4CCN(C[C@H]5CC[C@H](n6cc(NC(=O)c7coc(-c8ccnc(NCC(F)(F)F)c8)n7)c(C(F)F)n6)CC5)CC4)C3)cc2)C(=O)N1]

The title compound (5.0 mg, 32.1% yield) was prepared in a manner similar to at described in Example 1. ¹H NMR (500 MHz, DMSO) δ 10.25 (s, 1H), 9.73 (s, 1H), 8.96 (s, 1H), 8.24-8.18 (m, 2H), 7.64-7.61 (m, 1H), 7.27-7.05 (m, 5H), 6.43 (d, J=10.0 Hz, 2H), 4.26-4.21 (m, 3H), 3.90 (t, J=5.0 Hz, 2H), 3.67 (t, J=5.0 Hz, 2H), 3.57 (t, J=5.0 Hz, 2H), 3.25-3.22 (m, 2H), 2.67 (t, J=5.0 Hz, 2H), 2.43-2.25 (m, 7H), 2.12 (d, J=10.0 Hz, 2H), 2.04 (d, J=10.0 Hz, 2H), 1.90 (d, J=10.0 Hz, 2H), 1.77-1.74 (m, 2H), 1.65-1.55 (m, 1H), 1.05-1.03 (m, 2H). [M+H]⁺=826.6.

### Example 4: N-(3-(difluoromethyl)-1-((1r,4r)-4-((4-(1-(4-((R)-2,6-dioxopiperidin-3-yl)-3,5-difluorophenyl)azetidine-3-carbonyl)piperazin-1-yl)methyl)cyclohexyl)-1H-pyrazol-4-yl)-2-(2-((2,2,2-trifluoroethyl)amino)pyridin-4-yl)oxazole-4-carboxamide

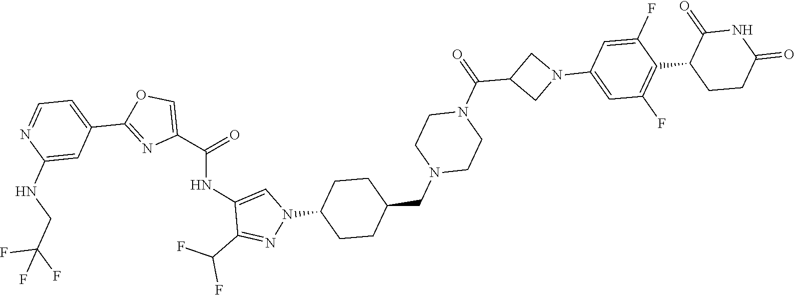 [SMILES: O=C1CC[C@H](c2c(F)cc(N3CC(C(=O)N4CCN(C[C@H]5CC[C@H](n6cc(NC(=O)c7coc(-c8ccnc(NCC(F)(F)F)c8)n7)c(C(F)F)n6)CC5)CC4)C3)cc2F)C(=O)N1]

The title compound was prepared in a manner similar to that described in Example 6.

¹H NMR (500 MHz, DMSO) δ 10.86 (s, 1H), 9.71 (s, 1H), 8.95 (s, 1H), 8.24 (d, J=5.0 Hz, 1H), 8.19 (s, 1H), 7.62-7.59 (m, 1H), 7.24 (s, 1H), 7.19-7.05 (m, 2H), 6.16 (d, J=10.0 Hz, 2H), 4.28-4.21 (m, 3H), 4.04-4.01 (m, 3H), 3.91-3.83 (m, 3H), 3.48 (s, 2H), 2.81-2.74 (m, 1H), 2.54-2.52 (m, 3H), 2.35-2.32 (m, 4H), 2.15-2.04 (m, 5H), 1.95-1.90 (m, 3H), 1.80-1.73 (m, 2H), 1.65-1.55 (m, 1H), 1.10-1.13 (m, 2H). [M+H]⁺=889.7.

### Example 5: N-(2-((1r,4r)-4-((4-(1-(4-(2,4-dioxotetrahydropyrimidin-1(2H)-yl)phenyl)azetidine-3-carbonyl)piperazin-1-yl)methyl)cyclohexyl)-6-methoxy-2H-indazol-5-yl)-6-(trifluoromethyl)picolinamide

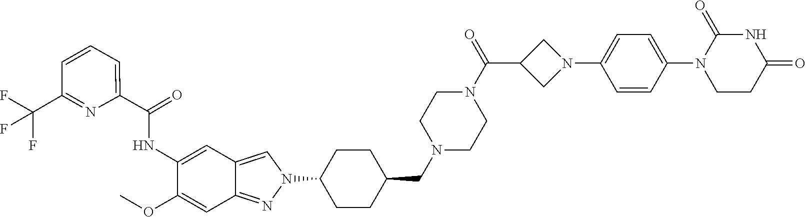 [SMILES: COc1cc2nn([C@H]3CC[C@H](CN4CCN(C(=O)C5CN(c6ccc(N7CCC(=O)NC7=O)cc6)C5)CC4)CC3)cc2cc1NC(=O)c1cccc(C(F)(F)F)n1]

The title compound was prepared in a manner similar to that described in Example 6.

¹H NMR (500 MHz, DMSO) δ 10.50 (s, 1H), 10.23 (s, 1H), 8.69 (s, 1H), 8.51-8.38 (m, 2H), 8.33 (s, 1H), 8.22 (d, J=7.7 Hz, 1H), 7.20-7.06 (m, 3H), 6.45 (d, J=8.4 Hz, 2H), 4.52-4.31 (m, 1H), 4.06-3.94 (m, 5H), 3.89-3.79 (m, 3H), 3.67 (t, J=6.5 Hz, 2H), 3.55-3.42 (m, 2H), 3.38-3.33 (m, 2H), 2.67 (t, J=6.5 Hz, 2H), 2.40-2.29 (m, 4H), 2.23-2.09 (m, 4H), 2.00-1.84 (m, 4H), 1.72-1.54 (m, 2H), 1.16-1.09 (m, 1H). [M+H]⁺=788.6.

### Example 6: N-(2-((1R,4r)-4-((4-((1r,4R)-4-(4-(2,4-dioxotetrahydropyrimidin-1(2H)-yl)phenoxy)cyclohexane-1-carbonyl)piperazin-1-yl)methyl)cyclohexyl)-6-methoxy-2H-indazol-5-yl)-6-(trifluoromethyl)picolinamide

### Step 1: tert-butyl 4-((1s,4s)-4-hydroxycyclohexane-1-carbonyl)piperazine-1-carboxylate

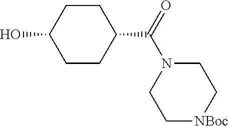 [SMILES: CC(C)(C)OC(=O)N1CCN(C(=O)[C@H]2CC[C@@H](O)CC2)CC1]

To a stirred solution of (1s,4s)-4-hydroxycyclohexane-1-carboxylic acid (20.0 g, 138.73 mmol) in THF (200 mL) was added HATU (68.6 g, 180.35 mmol) in portions at 0° C. The resulting solution was stirred for 30 min at 0° C. under nitrogen atmosphere. To the above solution were added TEA (28.1 g, 277.45 mmol, 38.62 mL) and tert-butyl piperazine-1-carboxylate (28.4 g, 152.60 mmol) at 0° C. under nitrogen atmosphere. The resulting solution was stirred for 12 hours at 20° C. The reaction mixture was quenched by addition H₂O 20 mL at 20° C., and then extracted with DCM (10 mL×3). The combined organic layers were washed with brine (10 mL*3), dried over Na₂SO₄, filtered and concentrated under reduced pressure to give a residue. The crude product was triturated with EA at 20° C. for 30 min, filtered and concentrated under reduced pressure to yield a residue compound tert-butyl 4-((1s,4s)-4-hydroxycyclohexane-1-carbonyl)piperazine-1-carboxylate (33.0 g, 75%). [M+H]⁺=313.3 ¹H NMR (400 MHz, CDCl₃) δ=4.02 (br d, J=2.9 Hz, 1H), 3.58 (br s, 3H), 3.51-3.36 (m, 6H), 2.58-2.45 (m, 1H), 2.05-1.82 (m, 5H), 1.77 (s, 1H), 1.66-1.57 (m, 3H), 1.50-1.44 (m, 9H)

### Step 2: tert-butyl 4-(((1s,4s)-4-hydroxycyclohexyl)methyl)piperazine-1-carboxylate

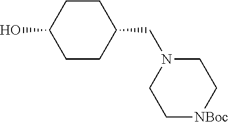 [SMILES: CC(C)(C)OC(=O)N1CCN(C[C@H]2CC[C@@H](O)CC2)CC1]

To a solution of tert-butyl 4-((1s,4s)-4-hydroxycyclohexane-1-carbonyl)piperazine-1-carboxylate (11.0 g, 35.2 mmol) in THF (120 mL) was added BH₃·THF (1 M, 140.8 mL) dropwise at 0° C. The solution was stirred at 80° C. for 16 hours. The resulting mixture was cooled to 0° C. followed by the addition of MeOH (2 mL) at 0° C. slowly. The mixture was stirred at 50° C. for 3 hours and concentrated under reduced pressure. The reaction mixture was quenched with H₂O (20 mL) at 20° C., and then extracted with DCM (10 mL*3). The combined organic layer was washed with brine (10 mL*3), dried over Na₂SO₄, filtered and concentrated under reduced pressure to afford the residue compound tert-butyl 4-(((1s,4s)-4-hydroxycyclohexyl)methyl)piperazine-1-carboxylate (10.0 g, 32%). [M+H]+=299.3 ¹H NMR (400 MHz, CHLOROFORM-d) δ=4.13 (q, J=7.2 Hz, 1H), 4.02-3.95 (m, 2H), 3.46-3.39 (m, 3H), 3.20-3.05 (m, 3H), 2.84-2.74 (m, 5H), 2.62 (s, 3H), 2.50-2.41 (m, 2H), 2.38-2.32 (m, 3H), 2.28 (d, J=6.7 Hz, 2H), 2.23-2.17 (m, 2H), 2.05 (s, 2H), 1.71 (td, J=4.1, 8.5 Hz, 7H), 1.49-1.43 (m, 9H), 1.43-1.33 (m, 5H), 1.26 (t, J=7.2 Hz, 2H)

### Step 3: tert-butyl 4-(((1s,4s)-4-((methylsulfonyl)oxy)cyclohexyl)methyl)piperazine-1-carboxylate

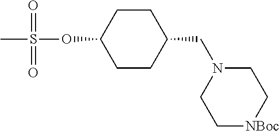 [SMILES: CC(C)(C)OC(=O)N1CCN(C[C@H]2CC[C@@H](OS(C)(=O)=O)CC2)CC1]

To a solution of tert-butyl 4-(((1s,4s)-4-hydroxycyclohexyl)methyl)piperazine-1-carboxylate (500 mg, 1.68 mmol) in DCM (5 mL) was added TEA (339 mg, 3.35 mmol, 466 uL) and MsCl (288 mg, 2.51 mmol, 195 uL) at 0° C. The mixture was stirred at 20° C. for 2 hours. The reaction mixture was quenched with aq. Na₂CO₃ (20 mL) at 20° C., and then extracted with DCM (10 mL*3). The combined organic layer was washed with brine (10 mL*3), dried over Na₂SO₄, filtered and concentrated under reduced pressure to afford a residue. The residue was purified on column chromatography (SiO₂, Petroleum ether/Ethyl acetate=40/1 to 0/1) to yield tert-butyl 4-(((1s,4s)-4-((methylsulfonyl)oxy)cyclohexyl)methyl)piperazine-1-carboxylate (280.0 mg, 44% yield). [M+H]⁺=377.3 ¹H NMR (400 MHz, CHLOROFORM-d) δ=4.97 (br s, 1H), 3.47-3.33 (m, 4H), 3.07-2.96 (m, 3H), 2.39-2.26 (m, 4H), 2.18 (d, J=7.1 Hz, 2H), 2.10-1.99 (m, 3H), 1.73-1.63 (m, 3H), 1.63-1.56 (m, 2H), 1.49-1.41 (m, 9H), 1.40-1.29 (m, 2H)

### Step 4: tert-butyl 4-(((1r,4r)-4-(6-methoxy-5-(6-(trifluoromethyl)picolinamido)-2H-indazol-2-yl)cyclohexyl)methyl)piperazine-1-carboxylate

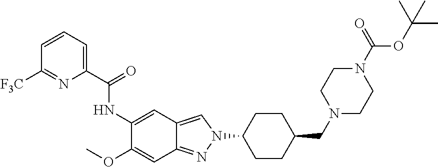 [SMILES: COc1cc2nn([C@H]3CC[C@H](CN4CCN(C(=O)OC(C)(C)C)CC4)CC3)cc2cc1NC(=O)c1cccc(C(F)(F)F)n1]

To a solution of N-(6-methoxy-2H-indazol-5-yl)-6-(trifluoromethyl)picolinamide (2.0 g, 5.95 mmol) (obtained through the same way described in Example 41) and tert-butyl 4-(((1s,4s)-4-((methylsulfonyl)oxy)cyclohexyl)methyl)piperazine-1-carboxylate (6.7 g, 17.84 mmol) in DMF (180 mL) was added Cs₂CO₃ (7.8 g, 23.79 mmol). The mixture was stirred at 80° C. for 12 hours. The reaction mixture was filtered and concentrated under reduced pressure to yield a residue. The residue was purified with prep-HPLC (water (NH₄HCO₃)-ACN]; B %: 55%-85%, 20 min). tert-butyl 4-(((1r,4r)-4-(6-methoxy-5-(6-(trifluoromethyl)picolinamido)-2H-indazol-2-yl)cyclohexyl)methyl)piperazine-1-carboxylate (300.0 mg, 7.85% yield) was obtained. [M+H]⁺=617.3 ¹H NMR (400 MHz, CHLOROFORM-d) δ=10.71 (s, 1H), 8.83 (s, 1H), 8.50 (d, J=7.9 Hz, 1H), 8.18-8.05 (m, 1H), 7.95-7.83 (m, 2H), 7.08 (s, 1H), 4.43-4.24 (m, 1H), 4.08-3.96 (m, 3H), 3.56-3.33 (m, 4H), 2.43-2.29 (m, 6H), 2.22 (d, J=7.0 Hz, 2H), 2.06 (br d, J=12.4 Hz, 2H), 1.94 (br dd, J=2.6, 12.5 Hz, 2H), 1.74-1.63 (m, 1H), 1.48 (s, 9H), 1.22-1.09 (m, 2H)

### Step 5: N-(6-methoxy-2-((1r,4r)-4-(piperazin-1-ylmethyl)cyclohexyl)-2H-indazol-5-yl)-6-(trifluoromethyl)picolinamide, TFA salt

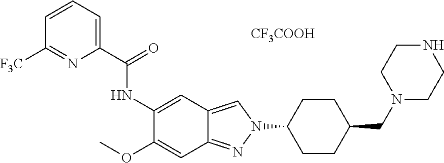 [SMILES: COc1cc2nn([C@H]3CC[C@H](CN4CCNCC4)CC3)cc2cc1NC(=O)c1cccc(C(F)(F)F)n1 ; "CF3COOH"]

A mixture of tert-butyl 4-(((1r,4r)-4-(6-methoxy-5-(6-(trifluoromethyl)picolinamido)-2H-indazol-2-yl)cyclohexyl)methyl)piperazine-1-carboxylate (280.0 mg, 454 umol), TFA (8.6 g, 75.6 mmol, 5.60 mL) in DCM (5 mL) was degassed and purged with N₂ for 3 times, and then the mixture was stirred at 20° C. for 2 hours under N₂ atmosphere. The reaction mixture was concentrated under reduced pressure to afford residue compound N-(6-methoxy-2-((1r,4r)-4-(piperazin-1-ylmethyl)cyclohexyl)-2H-indazol-5-yl)-6-(trifluoromethyl)picolinamide (TFA salt, 284 mg, 94%). [M+H]⁺=517.2 ¹H NMR (400 MHz, DMSO-d6) δ=10.55-10.47 (m, 1H), 9.52-9.33 (m, 1H), 8.72-8.66 (m, 1H), 8.47-8.36 (m, 2H), 8.32 (s, 1H), 8.20 (br d, J=7.5 Hz, 1H), 7.14 (s, 1H), 4.49-4.39 (m, 2H), 3.98 (s, 3H), 3.43 (br s, 6H), 3.04 (br d, J=5.2 Hz, 2H), 2.16 (br d, J=11.0 Hz, 2H), 2.03-1.84 (m, 6H), 1.31-1.16 (m, 3H).

### Step 6: N-(2-((1R,4r)-4-((4-((1r,4R)-4-(4-(2,4-dioxo-3-((2-(trimethylsilyl)ethoxy)methyl)tetrahydropyrimidin-1(2H)-yl)phenoxy)cyclohexane-1-carbonyl)piperazin-1-yl)methyl)cyclohexyl)-6-methoxy-2H-indazol-5-yl)-6-(trifluoromethyl)picolinamide

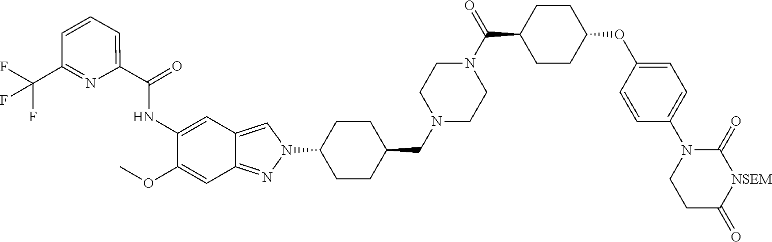 [SMILES: COc1cc2nn([C@H]3CC[C@H](CN4CCN(C(=O)[C@H]5CC[C@H](Oc6ccc(N7CCC(=O)N(COCC[Si](C)(C)C)C7=O)cc6)CC5)CC4)CC3)cc2cc1NC(=O)c1cccc(C(F)(F)F)n1]

To a solution of (trans)-4-(4-(2,4-dioxo-3-((2-(trimethylsilyl)ethoxy)methyl)tetrahydropyrimidin-1(2H)-yl)phenoxy)cyclohexane-1-carboxylic acid (obtained through the method similar to common intermediate B) (31 mg, 0.067 mmol) in DMF (3 mL) was added HATU (28 mg, 0.073 mmol). The mixture was stirred at rt for 10 min. Then, N-(6-methoxy-2-((1r,4r)-4-(piperazin-1-ylmethyl)cyclohexyl)-2H-indazol-5-yl)-6-(trifluoromethyl)picolinamide TFA salt (42 mg, 0.066 mmol) and Et₃N (46 mg, 0.45 mmol) in DMF (3 mL) was added to the reaction. The reaction was stirred at rt for 4 h. The solvent was removed by reduced pressure. The residue was diluted with water (15 mL), extracted with dichloromethane (3×20 mL), and washed with brine (30 mL). The organic phase was dried over anhydrous sodium sulfate, filtered and concentrated under reduced pressure to afford the crude product, which was purified with silica gel column chromatography (DCM:MeOH=100:0 to 19:1) to give the product (55 mg, 86% yield). [M+H]⁺=961.7.

### Step 7: N-(2-((1R,4r)-4-((4-((1r,4R)-4-(4-(2,4-dioxotetrahydropyrimidin-1(2H)-yl)phenoxy)cyclohexane-1-carbonyl)piperazin-1-yl)methyl)cyclohexyl)-6-methoxy-2H-indazol-5-yl)-6-(trifluoromethyl)picolinamide

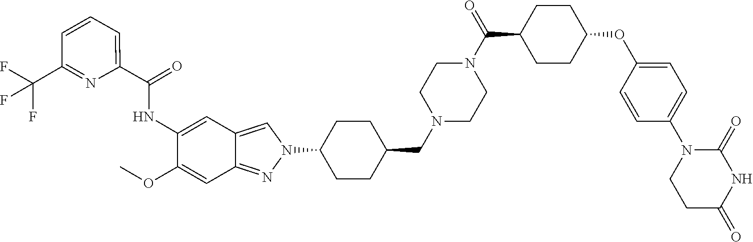 [SMILES: COc1cc2nn([C@H]3CC[C@H](CN4CCN(C(=O)[C@H]5CC[C@H](Oc6ccc(N7CCC(=O)NC7=O)cc6)CC5)CC4)CC3)cc2cc1NC(=O)c1cccc(C(F)(F)F)n1]

A solution of N-(2-((1R,4r)-4-((4-((1r,4R)-4-(4-(2,4-dioxo-3-((2-(trimethylsilyl)ethoxy)methyl)tetrahydropyrimidin-1(2H)-yl)phenoxy)cyclohexane-1-carbonyl)piperazin-1-yl)methyl)cyclohexyl)-6-methoxy-2H-indazol-5-yl)-6-(trifluoromethyl)picolinamide (50 mg, 0.052 mmol) in TFA (5 mL) was stirred at rt for 6 h. The solvent was removed by reduced pressure. The residue was dissolved in MeOH. The pH value of the mixture was adjusted to 1l with NH₃·H₂O (1N). The mixture was stirred at rt for 2 h. The mixture was concentrated under reduced pressure to afford the crude product, which was purified with silica gel column chromatography (DCM:MeOH=100:0˜9:1 gradient elution) to give the product (13 mg, 30% yield). [M+H]⁺=831.7

¹H NMR (500 MHz, DMSO) δ 10.50 (s, 1H), 10.29 (s, 1H), 8.69 (s, 1H), 8.42 (dt, J=7.7, 5.2 Hz, 2H), 8.33 (s, 1H), 8.22 (d, J=7.7 Hz, 1H), 7.21-7.16 (m, 3H), 6.95 (d, J=8.8 Hz, 2H), 4.53-4.25 (m, 2H), 3.98 (s, 3H), 3.71 (t, J=6.6 Hz, 2H), 3.56-3.41 (m, 4H), 2.73-2.61 (m, 3H), 2.40-2.23 (m, 5H), 2.19-2.06 (m, 5H), 2.00-1.86 (m, 4H), 1.77-1.71 (m, 2H), 1.68-1.39 (m, 6H), 1.16-1.08 (m, 2H).

### Example 7: N-(2-((1r,4r)-4-((4-(1-(4-(2,4-dioxotetrahydropyrimidin-1(2H)-yl)phenyl)piperidine-4-carbonyl)piperazin-1-yl)methyl)cyclohexyl)-6-methoxy-2H-indazol-5-yl)-6-(trifluoromethyl)picolinamide

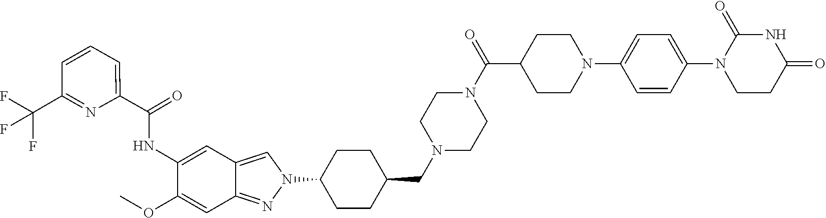 [SMILES: COc1cc2nn([C@H]3CC[C@H](CN4CCN(C(=O)C5CCN(c6ccc(N7CCC(=O)NC7=O)cc6)CC5)CC4)CC3)cc2cc1NC(=O)c1cccc(C(F)(F)F)n1]

The title compound was prepared in a manner similar to that described in Example 6.

¹H NMR (500 MHz, DMSO) δ 10.50 (s, 1H), 10.25 (s, 1H), 8.69 (s, 1H), 8.46 (d, J=7.6 Hz, 1H), 8.41 (t, J=7.7 Hz, 1H), 8.33 (s, 1H), 8.22 (d, J=7.8 Hz, 1H), 7.17-7.10 (m, 3H), 6.93 (d, J=8.8 Hz, 2H), 4.43-4.32 (m, 1H), 3.98 (s, 3H), 3.71-3.67 (m, 4H), 3.57-3.45 (m, 4H), 2.82-2.72 (m, 3H), 2.70-2.62 (m, 3H), 2.40-2.34 (m, 5H), 2.20-2.10 (m, 4H), 1.97-1.87 (m, 4H), 1.72-1.63 (m, 5H). [M+H]⁺=816.7.

### Example 12: N-(3-carbamoyl-1-((1R,4r)-4-(4-((R)-1-(4-((R)-2,6-dioxopiperidin-3-yl)-3,5-difluorophenyl)pyrrolidine-3-carbonyl)piperazin-1-yl)cyclohexyl)-1H-pyrazol-4-yl)-2-(2-((2,2,2-trifluoroethyl)amino)pyridin-4-yl)oxazole-4-carboxamide

### Step 1: tert-butyl 4-((1r,4r)-4-(4-(2-(2-((tert-butoxycarbonyl)(2,2,2-trifluoroethyl)amino)pyridin-4-yl)oxazole-4-carboxamido)-3-carbamoyl-1H-pyrazol-1-yl)cyclohexyl)piperazine-1-carboxylate

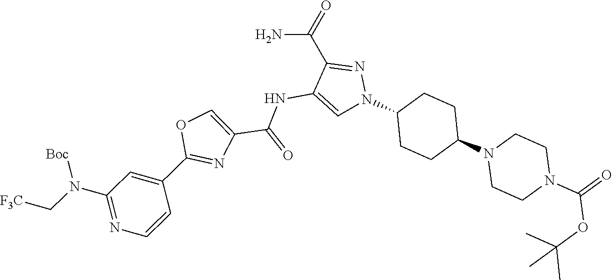 [SMILES: CC(C)(C)OC(=O)N1CCN([C@H]2CC[C@H](n3cc(NC(=O)c4coc(-c5ccnc(N(CC(F)(F)F)C(=O)OC(C)(C)C)c5)n4)c(C(N)=O)n3)CC2)CC1]

To a DMF solution (5 mL) of tert-butyl 4-((trans)-4-(4-amino-3-carbamoyl-1H-pyrazol-1-yl)cyclohexyl)piperazine-1-carboxylate (1.0 g, 2.54 mmol) was added 2-(2-((tert-butoxycarbonyl)(2,2,2-trifluoroethyl)amino)pyridin-4-yl)oxazole-4-carboxylic acid (1.2 g, 3.048 mmol), DIEA (518 mg, 5.08 mmol) and HATU (1.45 g, 5.08 mmol). The mixture was stirred overnight at room temperature. The mixture was purified with combi flash, eluted with DCM:MeOH=1-0 to 1:10 to afford the product (1.8 g, 93%). [M+H]⁺=761.8.

### Step 2: N-(3-carbamoyl-1-((1r,4r)-4-(piperazin-1-yl)cyclohexyl)-1H-pyrazol-4-yl)-2-(2-((2,2,2-trifluoroethyl)amino)pyridin-4-yl)oxazole-4-carboxamide

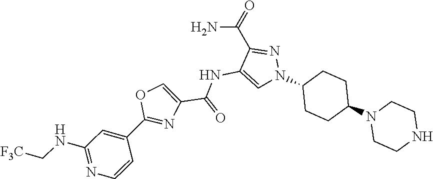 [SMILES: NC(=O)c1nn([C@H]2CC[C@H](N3CCNCC3)CC2)cc1NC(=O)c1coc(-c2ccnc(NCC(F)(F)F)c2)n1]

To a DCM solution (10 mL) of tert-butyl 4-((1r,4r)-4-(4-(2-(2-((tert-butoxycarbonyl)(2,2,2-trifluoroethyl)amino)pyridin-4-yl)oxazole-4-carboxamido)-3-carbamoyl-1H-pyrazol-1-yl)cyclohexyl)piperazine-1-carboxylate (1.8 g, 2.36 mmol) was added TFA (10 mL). The mixture was stirred overnight at room temperature. The solvent was removed under vacuum. The residue was basified to pH>7 with Na₂CO₃ (aq.), extracted with DCM (50 mL×3). The organic layer was dried over Na₂SO₄, filtered and concentrated to afford the crude product (1.2 g, 91%).

### Step 3: N-(3-carbamoyl-1-((1R,4r)-4-(4-((R)-1-(4-((R)-2,6-dioxopiperidin-3-yl)-3,5-difluorophenyl)pyrrolidine-3-carbonyl)piperazin-1-yl)cyclohexyl)-1H-pyrazol-4-yl)-2-(2-((2,2,2-trifluoroethyl)amino)pyridin-4-yl)oxazole-4-carboxamide

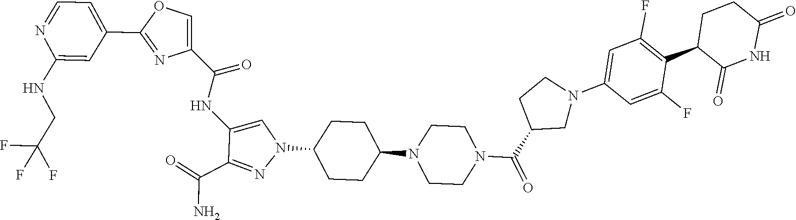 [SMILES: NC(=O)c1nn([C@H]2CC[C@H](N3CCN(C(=O)[C@@H]4CCN(c5cc(F)c([C@H]6CCC(=O)NC6=O)c(F)c5)C4)CC3)CC2)cc1NC(=O)c1coc(-c2ccnc(NCC(F)(F)F)c2)n1]

To a DMF solution (6 mL) of N-(3-carbamoyl-1-((1r,4r)-4-(piperazin-1-yl)cyclohexyl)-1H-pyrazol-4-yl)-2-(2-((2,2,2-trifluoroethyl)amino)pyridin-4-yl)oxazole-4-carboxamide (100 mg, 0.18 mmol) was added (R)-1-(4-((R)-2,6-dioxopiperidin-3-yl)-3,5-difluorophenyl)pyrrolidine-3-carboxylic acid (73 mg, 0.216 mmol) (obtained through the same way described in WO2022012623A1), DIEA (1.0 mL) and HATU (103 mg, 0.27 mmol). The mixture was stirred overnight at room temperature. The mixture was purified with combi flash, eluted with DCM:MeOH=1-0 to 1:10 to afford the crude product which was further purified with prepare HPLC to yield the product (57 mg, 36%). ¹H NMR (500 MHz, DMSO) δ 10.94 (s, 1H), 10.84 (s, 1H), 8.97 (s, 1H), 8.36 (s, 1H), 8.25 (d, J=5.0 Hz, 1H), 7.69-7.67 (m, 2H), 7.50 (s, 1H), 7.25 (s, 1H), 7.16 (d, J=5.0 Hz, 1H), 6.23 (d, J=10.0 Hz, 2H), 4.25-4.22 (m, 3H), 4.04-4.00 (m, 1H), 3.54-3.42 (m, 6H), 3.28-3.24 (m, 4H), 2.81-2.76 (m, 1H), 2.64-2.41 (m, 5H), 2.14-2.07 (m, 5H), 1.95-1.84 (m, 5H), 1.47-1.43 (m, 2H). [M+H]⁺=882.3.

### Example 13: N-(3-(difluoromethyl)-1-((1r,4r)-4-(4-(1-(4-(2,4-dioxotetrahydropyrimidin-1(2H)-yl)phenyl)azetidine-3-carbonyl)piperazin-1-yl)cyclohexyl)-1H-pyrazol-4-yl)-2-(2-((2,2,2-trifluoroethyl)amino)pyridin-4-yl)oxazole-4-carboxamide

### Step 1: tert-butyl 4-((1r,4r)-4-(4-(2-(2-((tert-butoxycarbonyl)(2,2,2-trifluoroethyl)amino)pyridin-4-yl)oxazole-4-carboxamido)-3-(difluoromethyl)-1H-pyrazol-1-yl)cyclohexyl)piperazine-1-carboxylate

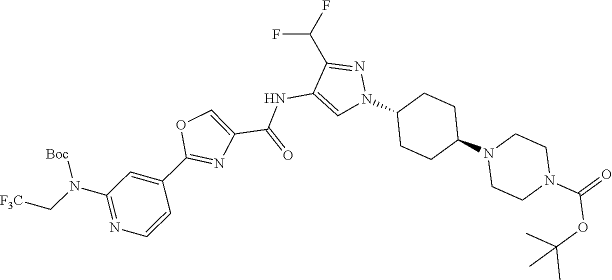 [SMILES: CC(C)(C)OC(=O)N1CCN([C@H]2CC[C@H](n3cc(NC(=O)c4coc(-c5ccnc(N(CC(F)(F)F)C(=O)OC(C)(C)C)c5)n4)c(C(F)F)n3)CC2)CC1]

To a DMF solution (10 mL) of tert-butyl 4-((trans)-4-(4-amino-3-(difluoromethyl)-1H-pyrazol-1-yl)cyclohexyl)piperazine-1-carboxylate (1.0 g, 2.54 mmol) was added 2-(2-((tert-butoxycarbonyl)(2,2,2-trifluoroethyl)amino)pyridin-4-yl)oxazole-4-carboxylic acid (1.2 g, 3.05 mmol), DIEA (518 mg, 5.08 mmol) and HATU (1.45 g, 3.81 mmol). The mixture was stirred overnight at room temperature. The mixture was purified with combi flash, eluted with DCM:MeOH=1-0 to 1:10 to afford the product (1.8 g, 92%). [M+H]⁺=769.4.

### Step 2: N-(3-(difluoromethyl)-1-((1r,4r)-4-(piperazin-1-yl)cyclohexyl)-1H-pyrazol-4-yl)-2-(2-((2,2,2-trifluoroethyl)amino)pyridin-4-yl)oxazole-4-carboxamide

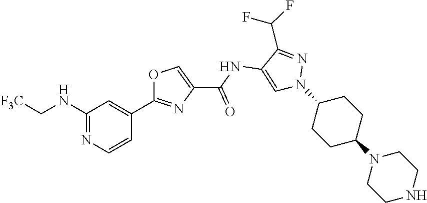 [SMILES: O=C(Nc1cn([C@H]2CC[C@H](N3CCNCC3)CC2)nc1C(F)F)c1coc(-c2ccnc(NCC(F)(F)F)c2)n1]

To a DCM solution (10 mL) of tert-butyl 4-((1r,4r)-4-(4-(2-(2-((tert-butoxycarbonyl)(2,2,2-trifluoroethyl)amino)pyridin-4-yl)oxazole-4-carboxamido)-3-(difluoromethyl)-1H-pyrazol-1-yl)cyclohexyl)piperazine-1-carboxylate (1.8 g, 2.34 mmol) was added TFA (5 mL). The mixture was stirred overnight at room temperature. The solvent was removed under vacuum. The residue was basified to pH 10 with Na₂CO₃ (aq.), extracted with DCM (50 mL×3). The organic layer was dried over Na₂SO₄, filtered and concentrated to afford the crude product (1.2 g, 90%).

### Step 3: N-(3-(difluoromethyl)-1-((1r,4r)-4-(4-(1-(4-(2,4-dioxotetrahydropyrimidin-1(2H)-yl)phenyl)azetidine-3-carbonyl)piperazin-1-yl)cyclohexyl)-1H-pyrazol-4-yl)-2-(2-((2,2,2-trifluoroethyl)amino)pyridin-4-yl)oxazole-4-carboxamide

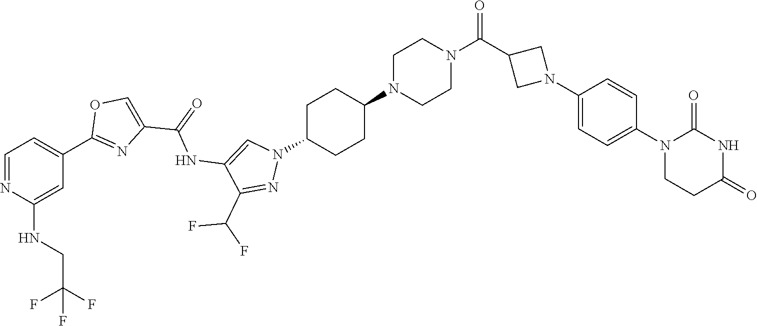 [SMILES: O=C1CCN(c2ccc(N3CC(C(=O)N4CCN([C@H]5CC[C@H](n6cc(NC(=O)c7coc(-c8ccnc(NCC(F)(F)F)c8)n7)c(C(F)F)n6)CC5)CC4)C3)cc2)C(=O)N1]

To a DCM solution (5 mL) of N-(3-(difluoromethyl)-1-((1r,4r)-4-(piperazin-1-yl)cyclohexyl)-1H-pyrazol-4-yl)-2-(2-((2,2,2-trifluoroethyl)amino)pyridin-4-yl)oxazole-4-carboxamide (50 mg, 0.088 mmol) was added 1-(4-(2,4-dioxotetrahydropyrimidin-1(2H)-yl)phenyl)azetidine-3-carboxylic acid (31 mg, 0.1056 mmol), DIEA (0.5 mL) and HATU (50 mg, 0.132 mmol). The mixture was stirred overnight at room temperature. The mixture was purified with combi flash, eluted with DCM:MeOH=1-0 to 1:10 to afford the crude product which was further purified with prep. HPLC to yield the product (14 mg, 19%). ¹H NMR (500 MHz, DMSO) δ 10.23 (s, 1H), 9.71 (s, 1H), 8.95 (s, 1H), 8.24 (d, J=5.0 Hz, 1H), 8.18 (s, 1H), 7.62-7.59 (m, 1H), 7.24 (s, 1H), 7.19-7.16 (m, 2H), 7.12-7.05 (m, 2H), 6.45 (d, J=10.0 Hz, 2H), 4.26-4.19 (m, 3H), 4.03-4.00 (m, 2H), 3.85-3.80 (m, 3H), 3.67 (t, J=10.0 Hz, 2H), 3.47 (s, 2H), 3.31-3.29 (m, 2H), 2.69-2.66 (m, 2H), 2.54-2.45 (m, 5H), 2.10-2.08 (m, 2H), 1.92-1.90 (m, 2H), 1.83-1.76 (m, 2H), 1.47-1.42 (m, 2H). [M+H]⁺=840.6.

### Example 14: N-(3-(difluoromethyl)-1-((1R,4r)-4-(4-((R)-1-(4-(2,4-dioxotetrahydropyrimidin-1(2H)-yl)phenyl)pyrrolidine-3-carbonyl)piperazin-1-yl)cyclohexyl)-1H-pyrazol-4-yl)-2-(2-((2,2,2-trifluoroethyl)amino)pyridin-4-yl)oxazole-4-carboxamide

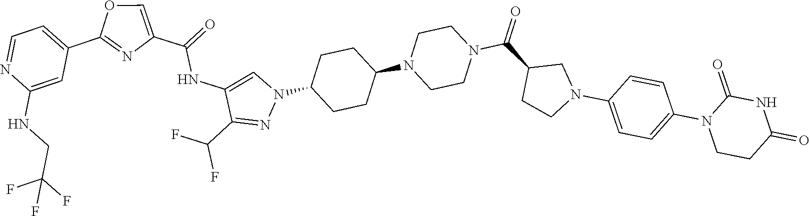 [SMILES: O=C1CCN(c2ccc(N3CC[C@@H](C(=O)N4CCN([C@H]5CC[C@H](n6cc(NC(=O)c7coc(-c8ccnc(NCC(F)(F)F)c8)n7)c(C(F)F)n6)CC5)CC4)C3)cc2)C(=O)N1]

The title compound was prepared in a manner similar to that described in Example 13.

¹H NMR (500 MHz, DMSO) δ 10.21 (s, 1H), 9.71 (s, 1H), 8.95 (s, 1H), 8.24 (d, J=5.0 Hz, 1H), 8.19 (s, 1H), 7.62-7.60 (m, 1H), 7.27-7.16 (m, 3H), 7.10 (d, J=10.0 Hz, 2H), 6.53 (d, J=10.0 Hz, 2H), 4.28-4.20 (m, 3H), 3.68-3.65 (m, 2H), 3.55-3.44 (m, 7H), 3.29-3.26 (m, 3H), 2.69-2.67 (m, 2H), 2.55 (s, 2H), 2.48-2.43 (m, 2H), 2.19-2.08 (m, 4H), 1.93-1.91 (m, 2H), 1.83-1.76 (m, 2H), 1.47-1.43 (m, 2H). [M+H]⁺=854.7.

### Example 15: N-(3-(difluoromethyl)-1-((1S,4r)-4-(4-((S)-1-(4-(2,4-dioxotetrahydropyrimidin-1(2H)-yl)phenyl)pyrrolidine-3-carbonyl)piperazin-1-yl)cyclohexyl)-1H-pyrazol-4-yl)-2-(2-((2,2,2-trifluoroethyl)amino)pyridin-4-yl)oxazole-4-carboxamide

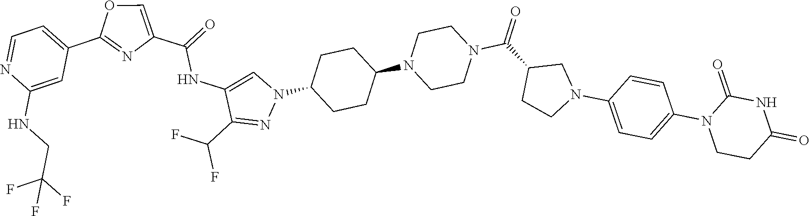 [SMILES: O=C1CCN(c2ccc(N3CC[C@H](C(=O)N4CCN([C@H]5CC[C@H](n6cc(NC(=O)c7coc(-c8ccnc(NCC(F)(F)F)c8)n7)c(C(F)F)n6)CC5)CC4)C3)cc2)C(=O)N1]

The title compound was prepared in a manner similar to that described in Example 13.

¹H NMR (500 MHz, DMSO) δ 10.21 (s, 1H), 9.72 (s, 1H), 8.95 (s, 1H), 8.24 (d, J=5.0 Hz, 1H), 8.19 (s, 1H), 7.62-7.60 (m, 1H), 7.27-7.16 (m, 3H), 7.10 (d, J=10.0 Hz, 2H), 6.53 (d, J=10.0 Hz, 2H), 4.26-4.22 (m, 3H), 3.68-3.65 (m, 2H), 3.48-3.44 (m, 6H), 3.35-3.26 (m, 4H), 2.69-2.66 (m, 2H), 2.55 (s, 2H), 2.48-2.43 (m, 2H), 2.16-2.08 (m, 4H), 1.93-1.91 (m, 2H), 1.83-1.76 (m, 2H), 1.47-1.43 (m, 2H). [M+H]⁺=854.7.

### Example 16: N-(3-(difluoromethyl)-1-((1r,4r)-4-(4-(1-(4-((R)-2,6-dioxopiperidin-3-yl)-3,5-difluorophenyl)azetidine-3-carbonyl)piperazin-1-yl)cyclohexyl)-1H-pyrazol-4-yl)-2-(2-((2,2,2-trifluoroethyl)amino)pyridin-4-yl)oxazole-4-carboxamide

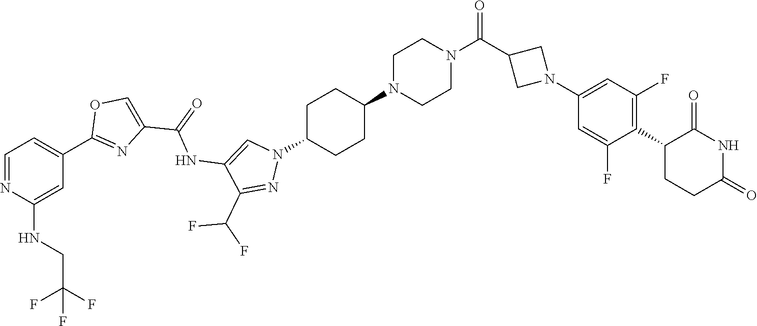 [SMILES: O=C1CC[C@H](c2c(F)cc(N3CC(C(=O)N4CCN([C@H]5CC[C@H](n6cc(NC(=O)c7coc(-c8ccnc(NCC(F)(F)F)c8)n7)c(C(F)F)n6)CC5)CC4)C3)cc2F)C(=O)N1]

The title compound was prepared in a manner similar to that described in Example 13.

¹H NMR (500 MHz, DMSO) δ 10.84 (s, 1H), 9.71 (s, 1H), 8.95 (s, 1H), 8.24 (d, J=5.0 Hz, 1H), 8.18 (s, 1H), 7.62-7.60 (m, 1H), 7.24-7.16 (m, 3H), 6.23 (d, J=10.0 Hz, 2H), 4.26-4.22 (m, 3H), 4.04-4.00 (m, 1H), 3.54-3.42 (m, 6H), 3.35-3.22 (m, 4H), 2.81-2.74 (m, 1H), 2.55 (s, 2H), 2.48-2.43 (m, 3H), 2.16-2.08 (m, 5H), 1.93-1.91 (m, 3H), 1.83-1.76 (m, 2H), 1.47-1.40 (m, 2H). [M+H]⁺=875.8.

### Example 17: N-(3-(difluoromethyl)-1-((1R,4r)-4-(4-((R)-1-(4-((R)-2,6-dioxopiperidin-3-yl)-3,5-difluorophenyl)pyrrolidine-3-carbonyl)piperazin-1-yl)cyclohexyl)-1H-pyrazol-4-yl)-2-(2-((2,2,2-trifluoroethyl)amino)pyridin-4-yl)oxazole-4-carboxamide

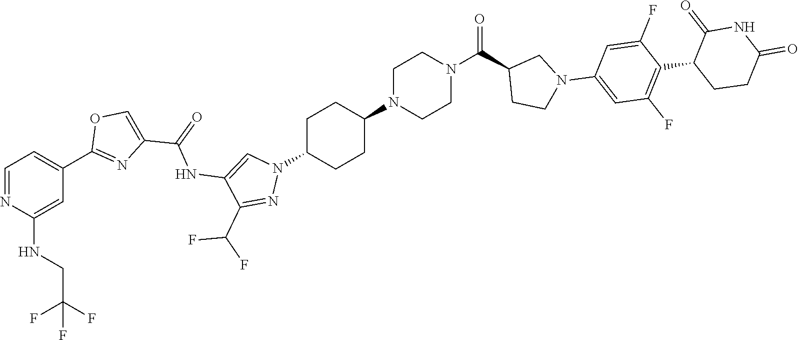 [SMILES: O=C1CC[C@H](c2c(F)cc(N3CC[C@@H](C(=O)N4CCN([C@H]5CC[C@H](n6cc(NC(=O)c7coc(-c8ccnc(NCC(F)(F)F)c8)n7)c(C(F)F)n6)CC5)CC4)C3)cc2F)C(=O)N1]

The title compound was prepared in a manner similar to that described in Example 13.

¹H NMR (500 MHz, DMSO) δ 10.87 (s, 1H), 9.73 (s, 1H), 8.99 (s, 1H), 8.24 (d, J=5.0 Hz, 1H), 8.18 (s, 1H), 7.62-7.60 (m, 1H), 7.24-7.06 (m, 3H), 6.18 (d, J=10.0 Hz, 2H), 4.26-4.03 (m, 4H), 3.58-3.43 (m, 11H), 2.84-2.71 (m, 2H), 2.09-1.82 (m, 13H), 1.45-1.41 (m, 2H). [M+H]⁺=889.7.

### Example 18: N-(3-(difluoromethyl)-1-((1R,4r)-4-(4-((R)-1-(4-((R)-2,6-dioxopiperidin-3-yl)-3,5-difluorophenyl)-4,4-dimethylpyrrolidine-3-carbonyl)piperazin-1-yl)cyclohexyl)-1H-pyrazol-4-yl)-2-(2-((2,2,2-trifluoroethyl)amino)pyridin-4-yl)oxazole-4-carboxamide

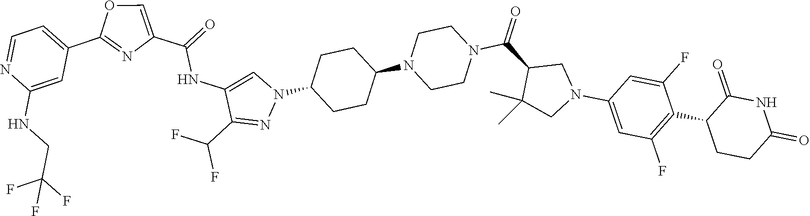 [SMILES: CC1(C)CN(c2cc(F)c([C@H]3CCC(=O)NC3=O)c(F)c2)C[C@@H]1C(=O)N1CCN([C@H]2CC[C@H](n3cc(NC(=O)c4coc(-c5ccnc(NCC(F)(F)F)c5)n4)c(C(F)F)n3)CC2)CC1]

The title compound was prepared in a manner similar to that described in Example 13.

¹H NMR (500 MHz, DMSO) δ 10.84 (s, 1H), 9.72 (s, 1H), 8.95 (s, 1H), 8.24 (d, J=5.0 Hz, 1H), 8.18 (s, 1H), 7.62-7.60 (m, 1H), 7.24-7.06 (m, 3H), 6.15 (d, J=10.0 Hz, 2H), 4.26-4.22 (m, 3H), 4.04-4.00 (m, 1H), 3.58-3.43 (m, 9H), 3.09-3.06 (m, 2H), 2.81-2.74 (m, 1H), 2.55-2.40 (m, 4H), 2.10-2.07 (m, 3H), 1.95-1.91 (m, 3H), 1.82-1.77 (m, 2H), 1.45-1.41 (m, 2H), 1.47 (s, 3H), 1.40 (s, 2H). [M+H]+=917.8.

### Example 19: N-(3-(difluoromethyl)-1-((1r,4r)-4-(4-((1-(4-((R)-2,6-dioxopiperidin-3-yl)-3,5-difluorophenyl)azetidin-3-yl)methyl)piperazin-1-yl)cyclohexyl)-1H-pyrazol-4-yl)-2-(2-((2,2,2-trifluoroethyl)amino)pyridin-4-yl)oxazole-4-carboxamide

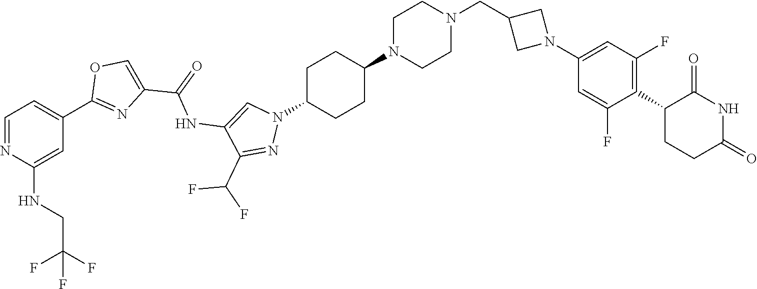 [SMILES: O=C1CC[C@H](c2c(F)cc(N3CC(CN4CCN([C@H]5CC[C@H](n6cc(NC(=O)c7coc(-c8ccnc(NCC(F)(F)F)c8)n7)c(C(F)F)n6)CC5)CC4)C3)cc2F)C(=O)N1]

The title compound was prepared in a manner similar to that described in Example 13.

¹H NMR (500 MHz, DMSO) δ 10.85 (s, 1H), 9.71 (s, 1H), 8.95 (s, 1H), 8.24 (d, J=5.0 Hz, 1H), 8.18 (s, 1H), 7.61-7.59 (m, 1H), 7.26-7.05 (m, 3H), 6.10 (d, J=10.0 Hz, 2H), 4.26-4.18 (m, 3H), 4.03-4.00 (m, 1H), 3.94-3.91 (m, 3H), 3.48-3.45 (m, 2H), 2.92-2.89 (m, 1H), 2.81-2.74 (m, 1H), 2.55-2.39 (m, 12H), 2.09-2.06 (m, 3H), 1.82-1.75 (m, 2H), 1.45-1.37 (m, 2H). [M+H]⁺=861.5.

### Example 20: N-(3-(difluoromethyl)-1-((1r,4r)-4-(4-((1-(4-((R)-2,6-dioxopiperidin-3-yl)-3,5-difluorophenyl)piperidin-4-yl)methyl)piperazin-1-yl)cyclohexyl)-1H-pyrazol-4-yl)-2-(2-((2,2,2-trifluoroethyl)amino)pyridin-4-yl)oxazole-4-carboxamide

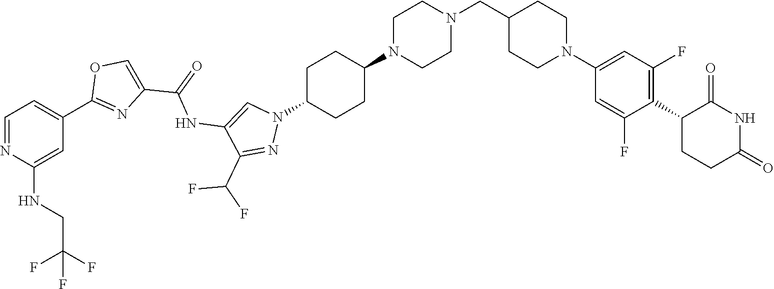 [SMILES: O=C1CC[C@H](c2c(F)cc(N3CCC(CN4CCN([C@H]5CC[C@H](n6cc(NC(=O)c7coc(-c8ccnc(NCC(F)(F)F)c8)n7)c(C(F)F)n6)CC5)CC4)CC3)cc2F)C(=O)N1]

The title compound was prepared in a manner similar to that described in Example 13.

¹H NMR (500 MHz, DMSO) δ 10.85 (s, 1H), 9.72 (s, 1H), 8.95 (s, 1H), 8.24 (d, J=5.0 Hz, 1H), 8.18 (s, 1H), 7.62-7.59 (m, 1H), 7.27-7.05 (m, 3H), 6.61 (d, J=10.0 Hz, 2H), 4.28-4.21 (m, 3H), 4.06-4.02 (m, 1H), 3.76-3.73 (m, 2H), 2.81-2.72 (m, 2H), 2.64-2.36 (m, 9H), 2.10-2.05 (m, 5H), 1.96-1.91 (m, 3H), 1.83-1.73 (m, 5H), 1.50-1.37 (m, 2H), 1.16-1.09 (m, 2H). [M+H]⁺=889.6

### Example 21: N-(3-(difluoromethyl)-1-((1r,4r)-4-(4-(1-(4-(2,4-dioxotetrahydropyrimidin-1(2H)-yl)phenyl)pyrrolidin-3-yl)piperazin-1-yl)cyclohexyl)-1H-pyrazol-4-yl)-2-(2-((2,2,2-trifluoroethyl)amino)pyridin-4-yl)oxazole-4-carboxamide

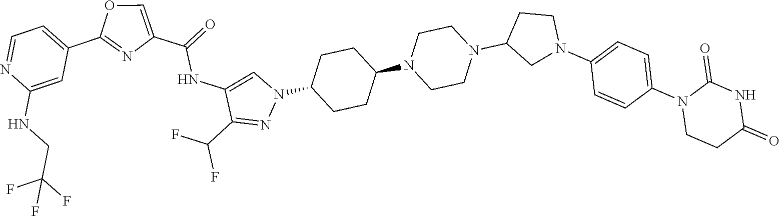 [SMILES: O=C1CCN(c2ccc(N3CCC(N4CCN([C@H]5CC[C@H](n6cc(NC(=O)c7coc(-c8ccnc(NCC(F)(F)F)c8)n7)c(C(F)F)n6)CC5)CC4)C3)cc2)C(=O)N1]

The title compound was prepared in a manner similar to that described in Example 13.

¹H NMR (500 MHz, DMSO) δ 10.20 (s, 1H), 9.71 (s, 1H), 8.95 (s, 1H), 8.24 (d, J=5.0 Hz, 1H), 8.18 (s, 1H), 7.62-7.59 (m, 1H), 7.27-7.05 (m, 5H), 6.52 (d, J=10.0 Hz, 2H), 4.26-4.19 (m, 3H), 3.67-3.65 (m, 2H), 3.47-3.43 (m, 1H), 3.37-3.32 (m, 4H), 3.23-3.21 (m, 1H), 3.05-3.02 (m, 1H), 2.90-2.87 (m, 1H), 2.67-2.64 (m, 2H), 2.54 (s, 4H), 2.36-2.33 (m, 2H), 2.18-2.07 (m, 3H), 1.94-1.92 (m, 2H), 1.83-1.75 (m, 3H), 1.44-1.37 (m, 2H). [M+H]⁺=826.6.

### Example 175: N-(3-(difluoromethyl)-1-((1r,4r)-4-(4-(1-(4-(2,6-dioxopiperidin-3-yl)phenyl)azetidine-3-carbonyl)piperazin-1-yl)cyclohexyl)-1H-pyrazol-4-yl)-2-(2-((2,2,2-trifluoroethyl)amino)pyridin-4-yl)oxazole-4-carboxamide

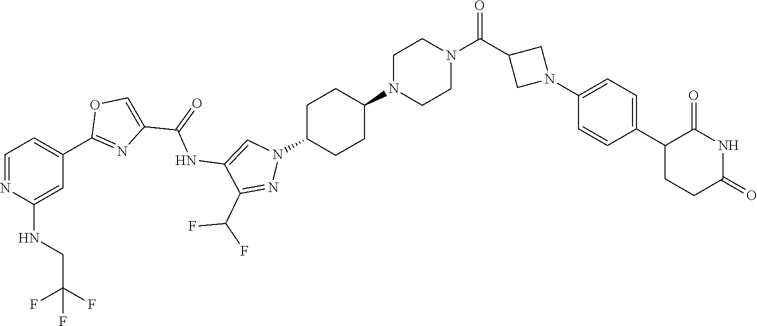 [SMILES: O=C1CCC(c2ccc(N3CC(C(=O)N4CCN([C@H]5CC[C@H](n6cc(NC(=O)c7coc(-c8ccnc(NCC(F)(F)F)c8)n7)c(C(F)F)n6)CC5)CC4)C3)cc2)C(=O)N1]

The title compound was prepared in a manner similar to that described in Example 13.

¹H NMR (500 MHz, DMSO) δ 10.75 (s, 1H), 9.71 (s, 1H), 8.95 (s, 1H), 8.24 (d, J=5.0 Hz, 1H), 8.18 (s, 1H), 7.61 (t, J=5.0 Hz, 1H), 7.24-7.16 (m, 3H), 7.01 (d, J=10.0 Hz, 2H), 6.42 (d, J=10.0 Hz, 2H), 4.28-4.19 (m, 3H), 4.01-3.99 (m, 2H), 3.84-3.79 (m, 3H), 3.72-3.69 (m, 1H), 3.59 (t, J=5.0 Hz, 2H), 3.47 (s, 2H), 2.66-2.60 (m, 1H), 2.54-2.44 (m, 8H), 2.12-2.07 (m, 3H), 2.01-1.98 (m, 1H), 1.91-1.89 (m, 2H), 1.83-1.76 (m, 2H), 1.46-1.39 (m, 2H). [M+H]⁺=839.6.

### Example 22: 2-(2-((cyclopropylmethyl)amino)pyridin-4-yl)-N-(3-(difluoromethyl)-1-((1S,4r)-4-(4-((S)-1-(4-(2,4-dioxotetrahydropyrimidin-1(2H)-yl)phenyl)pyrrolidine-3-carbonyl)piperazin-1-yl)cyclohexyl)-1H-pyrazol-4-yl)oxazole-4-carboxamide

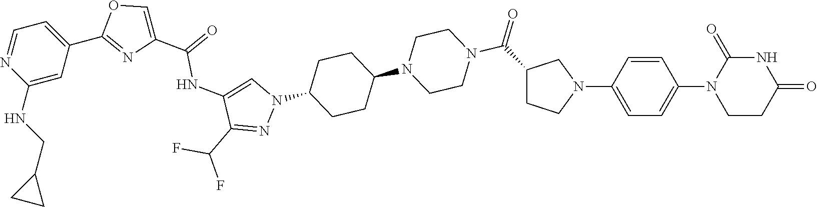 [SMILES: O=C1CCN(c2ccc(N3CC[C@H](C(=O)N4CCN([C@H]5CC[C@H](n6cc(NC(=O)c7coc(-c8ccnc(NCC9CC9)c8)n7)c(C(F)F)n6)CC5)CC4)C3)cc2)C(=O)N1]

The title compound was prepared in a manner similar to that described in Example 13.

¹H NMR (500 MHz, DMSO) δ 10.21 (s, 1H), 9.70 (s, 1H), 8.92 (s, 1H), 8.18 (s, 1H), 8.15 (d, J=5.0 Hz, 1H), 7.27-7.01 (m, 6H), 6.53 (d, J=10.0 Hz, 2H), 4.22-4.19 (m, 1H), 3.68-3.65 (m, 2H), 3.55-3.44 (m, 6H), 3.35-3.24 (m, 4H), 3.18 (t, J=5.0 Hz, 2H), 2.68 (t, J=5.0 Hz, 2H), 2.57-2.55 (m, 1H), 2.48-2.42 (m, 3H), 2.16-2.08 (m, 4H), 1.93-1.91 (m, 2H), 1.83-1.76 (m, 2H), 1.47-1.43 (m, 2H), 1.08-1.05 (m, 1H), 0.47-0.43 (m, 2H), 0.24-0.21 (m, 2H). [M+H]⁺=826.9.

### Example 23: 2-(2-((cyclopropylmethyl)amino)pyridin-4-yl)-N-(3-(difluoromethyl)-1-((1R,4r)-4-(4-((R)-1-(4-(2,4-dioxotetrahydropyrimidin-1(2H)-yl)phenyl)pyrrolidine-3-carbonyl)piperazin-1-yl)cyclohexyl)-1H-pyrazol-4-yl)oxazole-4-carboxamide

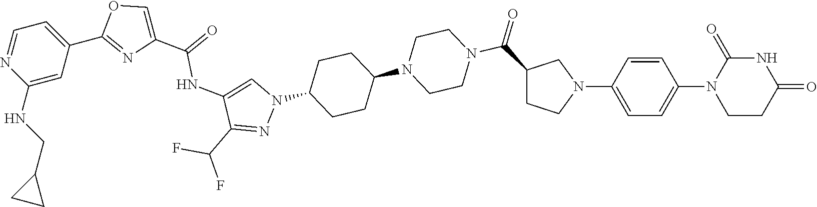 [SMILES: O=C1CCN(c2ccc(N3CC[C@@H](C(=O)N4CCN([C@H]5CC[C@H](n6cc(NC(=O)c7coc(-c8ccnc(NCC9CC9)c8)n7)c(C(F)F)n6)CC5)CC4)C3)cc2)C(=O)N1]

The title compound was prepared in a manner similar to that described in Example 13.

¹H NMR (500 MHz, DMSO) δ 10.21 (s, 1H), 9.70 (s, 1H), 8.92 (s, 1H), 8.18 (s, 1H), 8.15 (d, J=5.0 Hz, 1H), 7.27-7.01 (m, 6H), 6.53 (d, J=10.0 Hz, 2H), 4.24-4.19 (m, 1H), 3.68-3.65 (m, 2H), 3.56-3.44 (m, 6H), 3.35-3.24 (m, 4H), 3.18 (t, J=5.0 Hz, 2H), 2.68 (t, J=5.0 Hz, 2H), 2.57-2.55 (m, 1H), 2.48-2.42 (m, 3H), 2.16-2.08 (m, 4H), 1.93-1.91 (m, 2H), 1.83-1.76 (m, 2H), 1.47-1.40 (m, 2H), 1.08-1.05 (m, 1H), 0.47-0.43 (m, 2H), 0.24-0.21 (m, 2H). [M+H]⁺=826.9.

### Example 24: 2-(2-((cyclopropylmethyl)amino)pyridin-4-yl)-N-(3-(difluoromethyl)-1-((1r,4r)-4-(4-(1-(4-(2,4-dioxotetrahydropyrimidin-1(2H)-yl)phenyl)azetidine-3-carbonyl)piperazin-1-yl)cyclohexyl)-1H-pyrazol-4-yl)oxazole-4-carboxamide

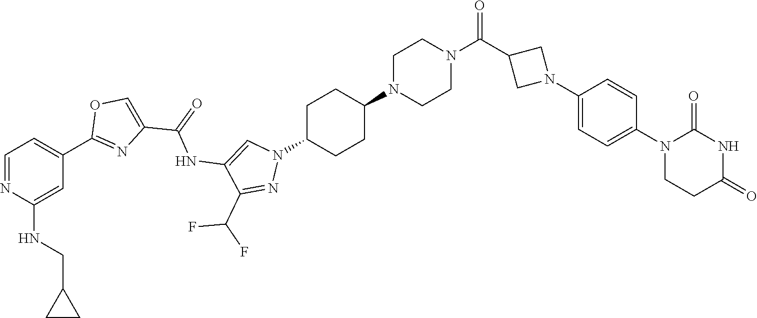 [SMILES: O=C1CCN(c2ccc(N3CC(C(=O)N4CCN([C@H]5CC[C@H](n6cc(NC(=O)c7coc(-c8ccnc(NCC9CC9)c8)n7)c(C(F)F)n6)CC5)CC4)C3)cc2)C(=O)N1]

The title compound was prepared in a manner similar to that described in Example 13.

¹H NMR (500 MHz, DMSO) δ 10.23 (s, 1H), 9.70 (s, 1H), 8.92 (s, 1H), 8.18 (s, 1H), 8.15 (d, J=5.0 Hz, 1H), 7.27-7.01 (m, 6H), 6.45 (d, J=10.0 Hz, 2H), 4.24-4.19 (m, 1H), 4.03-4.00 (m, 2H), 3.87-3.81 (m, 3H), 3.67 (t, J=5.0 Hz, 2H), 3.47 (s, 2H), 3.18 (t, J=5.0 Hz, 2H), 2.67 (t, J=5.0 Hz, 2H), 2.57-2.42 (m, 7H), 2.10-2.08 (m, 2H), 1.92-1.90 (m, 2H), 1.83-1.76 (m, 2H), 1.45-1.42 (m, 2H), 1.08-1.05 (m, 1H), 0.47-0.44 (m, 2H), 0.23-0.21 (m, 2H). [M+H]⁺=812.6.

### Example 25: 2-(2-((cyclopropylmethyl)amino)pyridin-4-yl)-N-(3-(difluoromethyl)-1-((1r,4r)-4-(4-(1-(4-((R)-2,6-dioxopiperidin-3-yl)-3,5-difluorophenyl)azetidine-3-carbonyl)piperazin-1-yl)cyclohexyl)-1H-pyrazol-4-yl)oxazole-4-carboxamide

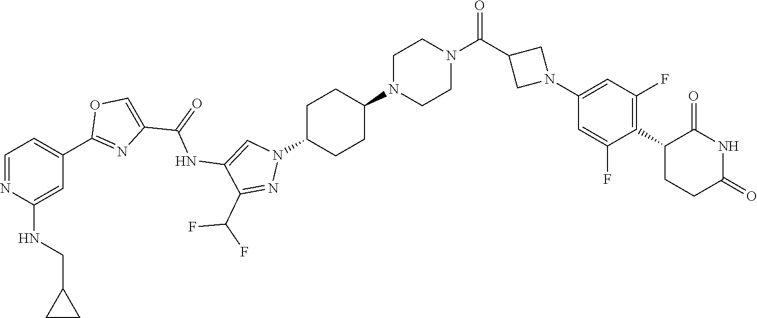 [SMILES: O=C1CC[C@H](c2c(F)cc(N3CC(C(=O)N4CCN([C@H]5CC[C@H](n6cc(NC(=O)c7coc(-c8ccnc(NCC9CC9)c8)n7)c(C(F)F)n6)CC5)CC4)C3)cc2F)C(=O)N1]

The title compound was prepared in a manner similar to that described in Example 13.

¹H NMR (500 MHz, DMSO) δ 10.87 (s, 1H), 9.92-9.81 (m, 2H), 9.01 (s, 1H), 8.22 (s, 1H), 8.13 (d, J=5.0 Hz, 1H), 7.35-7.07 (m, 3H), 6.17 (d, J=10.0 Hz, 2H), 4.54-4.52 (m, 1H), 4.33-4.30 (m, 1H), 4.06-3.83 (m, 7H), 3.49-3.40 (m, 4H), 3.24-3.02 (m, 5H), 2.82-2.75 (m, 1H), 2.21-2.04 (m, 5H), 1.96-1.85 (m, 3H), 1.71-1.64 (m, 2H), 1.12-1.08 (m, 1H), 0.52-0.51 (m, 2H), 0.28-0.27 (m, 2H). [M+H]⁺=848.0.

### Example 26: 2-(2-((cyclopropylmethyl)amino)pyridin-4-yl)-N-(3-(difluoromethyl)-1-((1R,4r)-4-(4-((R)-1-(4-((R)-2,6-dioxopiperidin-3-yl)-3,5-difluorophenyl)pyrrolidine-3-carbonyl)piperazin-1-yl)cyclohexyl)-1H-pyrazol-4-yl)oxazole-4-carboxamide

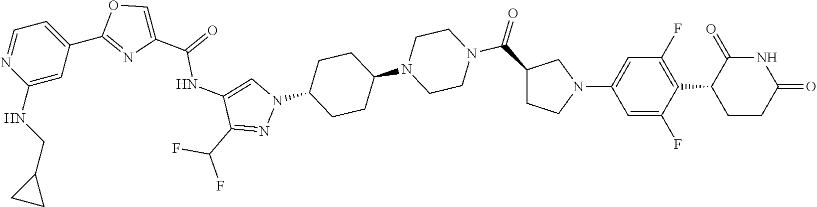 [SMILES: O=C1CC[C@H](c2c(F)cc(N3CC[C@@H](C(=O)N4CCN([C@H]5CC[C@H](n6cc(NC(=O)c7coc(-c8ccnc(NCC9CC9)c8)n7)c(C(F)F)n6)CC5)CC4)C3)cc2F)C(=O)N1]

The title compound was prepared in a manner similar to that described in Example 13.

¹H NMR (500 MHz, DMSO) δ 10.84 (s, 1H), 9.70 (s, 1H), 8.92 (s, 1H), 8.18 (s, 1H), 8.15 (d, J=5.0 Hz, 1H), 7.27-7.01 (m, 4H), 6.23 (d, J=10.0 Hz, 2H), 4.24-4.19 (m, 1H), 4.04-4.00 (m, 1H), 3.54-3.42 (m, 6H), 3.35-3.22 (m, 5H), 3.19-3.17 (m, 2H), 2.81-2.74 (m, 1H), 2.55 (s, 2H), 2.48-2.43 (m, 2H), 2.18-2.05 (m, 5H), 1.95-1.91 (m, 3H), 1.83-1.76 (m, 2H), 1.47-1.40 (m, 2H), 1.09-1.04 (m, 1H), 0.47-0.43 (m, 2H), 0.24-0.21 (m, 2H). [M+H]⁺=861.7.

### Example 27: 2-(2-((cyclopropylmethyl)amino)pyridin-4-yl)-N-(3-(difluoromethyl)-1-((1r,4r)-4-(4-((1-(4-((R)-2,6-dioxopiperidin-3-yl)-3,5-difluorophenyl)azetidin-3-yl)methyl)piperazin-1-yl)cyclohexyl)-1H-pyrazol-4-yl)oxazole-4-carboxamide

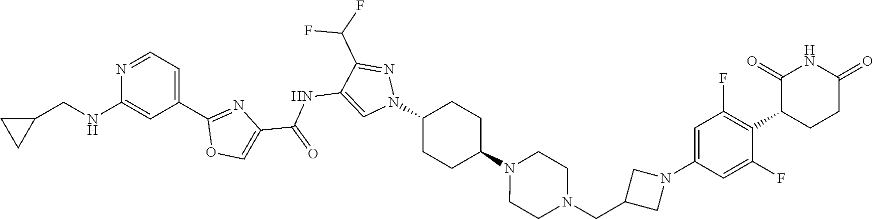 [SMILES: O=C1CC[C@H](c2c(F)cc(N3CC(CN4CCN([C@H]5CC[C@H](n6cc(NC(=O)c7coc(-c8ccnc(NCC9CC9)c8)n7)c(C(F)F)n6)CC5)CC4)C3)cc2F)C(=O)N1]

The title compound was prepared in a manner similar to that described in Example 13.

¹H NMR (500 MHz, DMSO) δ 10.85 (s, 1H), 9.70 (s, 1H), 8.92 (s, 1H), 8.18 (s, 1H), 8.15 (d, J=5.0 Hz, 1H), 7.26-7.01 (m, 4H), 6.10 (d, J=10.0 Hz, 2H), 4.24-4.19 (m, 1H), 4.04-4.00 (m, 1H), 3.94-3.91 (m, 2H), 3.48-3.46 (m, 2H), 3.19-3.17 (m, 2H), 2.92-2.91 (m, 1H), 2.77-2.74 (m, 1H), 2.63-2.36 (m, 12H), 2.18-2.05 (m, 3H), 1.95-1.93 (m, 3H), 1.82-1.75 (m, 2H), 1.45-1.40 (m, 2H), 1.08-1.05 (m, 1H), 0.46-0.43 (m, 2H), 0.23-0.21 (m, 2H). [M+H]⁺=833.7.

### Example 85: N-(3-(difluoromethyl)-1-((1R,4r)-4-(4-((R)-1-(4-((R)-2,6-dioxopiperidin-3-yl)-3,5-difluorophenyl)pyrrolidine-3-carbonyl)piperazin-1-yl)cyclohexyl)-1H-pyrazol-4-yl)-2-(2-isopropylpyridin-4-yl)oxazole-4-carboxamide

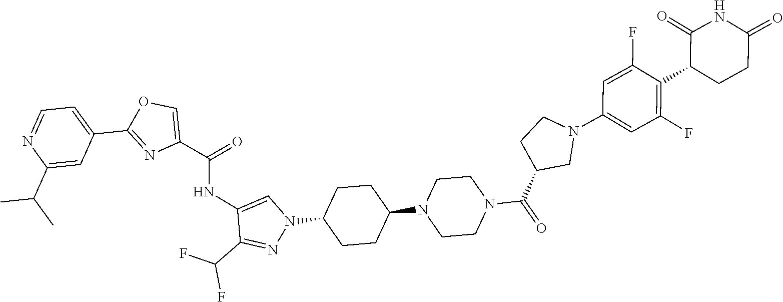 [SMILES: CC(C)c1cc(-c2nc(C(=O)Nc3cn([C@H]4CC[C@H](N5CCN(C(=O)[C@@H]6CCN(c7cc(F)c([C@H]8CCC(=O)NC8=O)c(F)c7)C6)CC5)CC4)nc3C(F)F)co2)ccn1]

The title compound was prepared in a manner similar to that described in Example 13.

¹H NMR (500 MHz, DMSO) δ 10.84 (s, 1H), 9.85 (s, 1H), 8.99 (d, J=2.3 Hz, 1H), 8.73 (s, 1H), 7.15 (t, J=54.6 Hz, 1H), 6.22 (d, J=13.4 Hz, 2H), 4.26-4.20 (m, 1H), 4.10-3.95 (m, 1H), 3.67-3.39 (m, 8H), 3.30-2.99 (m, 4H), 2.84-2.61 (m, 2H), 2.58-2.49 (m, 3H), 2.24-2.01 (m, 5H), 1.97-1.86 (brs, 3H), 1.82-1.73 (m, 2H), 1.48-1.31 (m, 2H), 1.28 (d J=15.1 Hz, 6H). [M+H]⁺=834.6

### Example 86: N-(3-(difluoromethyl)-1-((1r,4r)-4-(4-(1-(4-((R)-2,6-dioxopiperidin-3-yl)-3,5-difluorophenyl)azetidine-3-carbonyl)piperazin-1-yl)cyclohexyl)-1H-pyrazol-4-yl)-2-(2-isopropylpyridin-4-yl)oxazole-4-carboxamide

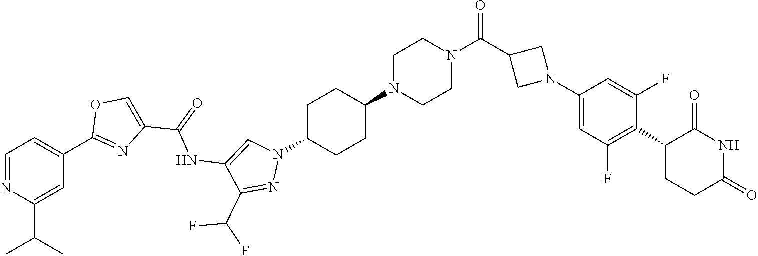 [SMILES: CC(C)c1cc(-c2nc(C(=O)Nc3cn([C@H]4CC[C@H](N5CCN(C(=O)C6CN(c7cc(F)c([C@H]8CCC(=O)NC8=O)c(F)c7)C6)CC5)CC4)nc3C(F)F)co2)ccn1]

The title compound was prepared in a manner similar to that described in Example 13.

¹H NMR (500 MHz, DMSO) δ 10.86 (s, 1H), 9.85 (s, 1H), 8.99 (s, 1H), 8.73 (d, J=5.0 Hz, 1H), 8.15 (s, 1H), 7.84 (s, 1H), 7.78 (d, J=5.0 Hz, 1H), 7.15 (t, J=54.4 Hz, 1H), 6.17 (d, J=11.1 Hz, 2H), 4.21 (s, 1H), 4.03 (t, J=7.3 Hz, 3H), 3.91 (d, J=6.2 Hz, 2H), 3.89-3.78 (m, 1H), 3.47 (s, 2H), 3.38-3.24 (m, 3H), 3.23-3.10 (m, 1H), 2.88 (s, 1H), 2.84-2.71 (m, 1H), 2.48-2.36 (m, 3H), 2.19-2.04 (m, 3H), 1.96-1.81 (m, 3H), 1.80-1.73 (m, 2H), 1.43 (d, J=11.5 Hz, 2H), 1.29 (d, J=6.9 Hz, 6H). [M+H]⁺=820.7

### Example 170: 2-(2-(sec-butyl)pyridin-4-yl)-N-(3-(difluoromethyl)-1-((1R,4r)-4-(4-((R)-1-(4-((R)-2,6-dioxopiperidin-3-yl)-3,5-difluorophenyl)pyrrolidine-3-carbonyl)piperazin-1-yl)cyclohexyl)-1H-pyrazol-4-yl)oxazole-4-carboxamide

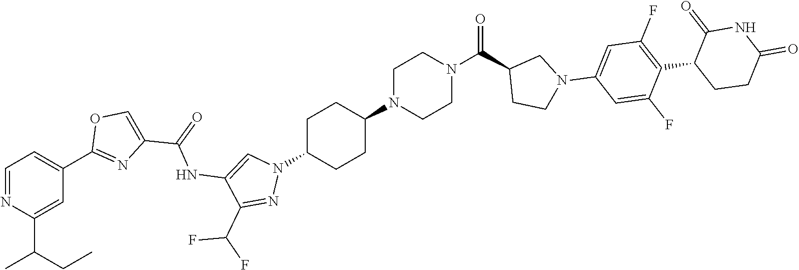 [SMILES: CCC(C)c1cc(-c2nc(C(=O)Nc3cn([C@H]4CC[C@H](N5CCN(C(=O)[C@@H]6CCN(c7cc(F)c([C@H]8CCC(=O)NC8=O)c(F)c7)C6)CC5)CC4)nc3C(F)F)co2)ccn1]

The title compound was prepared in a manner similar to that described in Example 13.

¹H NMR (500 MHz, DMSO) δ 10.84 (s, 1H), 9.85 (s, 1H), 8.99 (s, 1H), 8.75 (d, J=5.0 Hz, 1H), 8.15 (s, 1H), 7.86-7.73 (m, 2H), 7.15 (t, J=54.4 Hz, 1H), 6.22 (d, J=12.2 Hz, 2H), 4.22 (brs, 1H), 4.02 (dd, J=12.5, 4.8 Hz, 1H), 3.66-3.35 (m, 6H), 3.28-3.15 (m, 5H), 2.97-2.89 (m, 1H), 2.86-2.73 (m, 2H), 2.54 (s, 2H), 2.22-2.00 (m, 6H), 1.93 (brs, 3H), 1.85-1.70 (m, 3H), 1.69-1.60 (m, 1H), 1.52-1.39 (m, 2H), 1.32-1.20 (m, 4H), 0.80 (t, J=7.4 Hz, 3H). [M+H]⁺=848.6

### Example 28: N-(3-carbamoyl-1-(6-(4-(1-(4-(2,4-dioxotetrahydropyrimidin-1(2H)-yl)phenyl)azetidine-3-carbonyl)piperazin-1-yl)pyridin-3-yl)-1H-pyrazol-4-yl)-2-(2-((2,2,2-trifluoroethyl)amino)pyridin-4-yl)oxazole-4-carboxamide

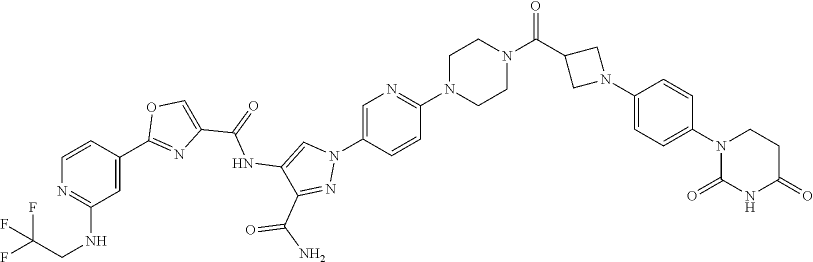 [SMILES: NC(=O)c1nn(-c2ccc(N3CCN(C(=O)C4CN(c5ccc(N6CCC(=O)NC6=O)cc5)C4)CC3)nc2)cc1NC(=O)c1coc(-c2ccnc(NCC(F)(F)F)c2)n1]

The title compound was prepared in a manner similar to that described in Example 166.

¹H NMR (500 MHz, DMSO) δ 10.95 (s, 1H), 10.19 (s, 1H), 8.96 (s, 1H), 8.79 (s, 1H), 8.65 (d, J=5.0 Hz, 1H), 8.19 (d, J=5.0 Hz, 1H), 8.08-8.06 (m, 1H), 7.96 (s, 1H), 7.64 (s, 2H), 7.20 (s, 1H), 7.11 (d, J=10.0 Hz, 2H), 7.05 (d, J=10.0 Hz, 2H), 6.98 (d, J=10.0 Hz, 1H), 6.40 (d, J=10.0 Hz, 2H), 4.20-4.16 (m, 2H), 4.01-3.98 (m, 2H), 3.87-3.85 (m, 3H), 3.62-3.59 (m, 2H), 3.56-3.53 (m, 6H), 3.44-3.40 (m, 2H), 2.62-2.59 (m, 2H). [M+H]⁺=828.6.

### Example 8: N-(3-carbamoyl-1-((1r,4r)-4-(4-(1-(4-(2,4-dioxotetrahydropyrimidin-1(2H)-yl)phenyl)azetidine-3-carbonyl)piperazin-1-yl)cyclohexyl)-1H-pyrazol-4-yl)-2-(2-((2,2,2-trifluoroethyl)amino)pyridin-4-yl)oxazole-4-carboxamide

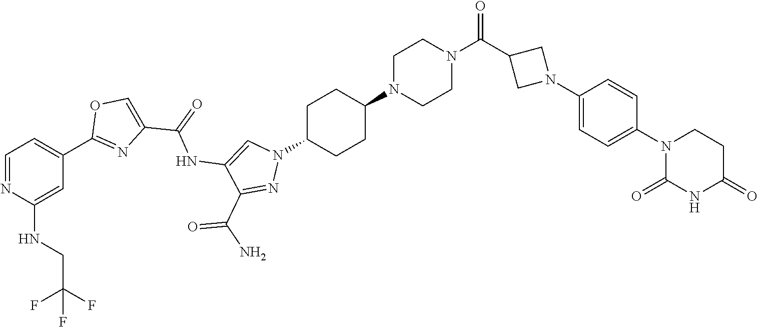 [SMILES: NC(=O)c1nn([C@H]2CC[C@H](N3CCN(C(=O)C4CN(c5ccc(N6CCC(=O)NC6=O)cc5)C4)CC3)CC2)cc1NC(=O)c1coc(-c2ccnc(NCC(F)(F)F)c2)n1]

To a solution of N-(3-carbamoyl-1-((1r,4r)-4-(piperazin-1-yl)cyclohexyl)-1H-pyrazol-4-yl)-2-(2-((2,2,2-trifluoroethyl)amino)pyridin-4-yl)oxazole-4-carboxamide (56 mg, 0.1 mmol), 1-(4-(2,4-dioxotetrahydropyrimidin-1(2H)-yl)phenyl)azetidine-3-carboxylic acid (30 mg, 0.1 mmol) and DIEA (65 mg, 0.5 mmol) in DMF (5 mL) was added HATU (38 mg, 0.1 mmol) at room temperature. The resulting mixture was stirred at room temperature for 18 hours. The reaction was evaporated under vacuum to afford the crude residue, which was purified with Prep-TLC (DCM:MeOH=7:1) to afford product (41 mg, 49%). ¹H NMR (500 MHz, DMSO) δ 10.94 (s, 1H), 10.23 (s, 1H), 8.97 (s, 1H), 8.36 (s, 1H), 8.24 (d, J=5.5 Hz, 1H), 7.71-7.64 (m, 2H), 7.50 (s, 1H), 7.25 (s, 1H), 7.16 (d, J=6.0 Hz, 1H), 7.11 (d, J=8.5 Hz, 2H), 6.45 (d, J=8.5 Hz, 2H), 4.28-4.19 (m, 3H), 4.04-3.98 (m, 2H), 3.89-3.78 (m, 3H), 3.71-3.63 (m, 2H), 3.51-3.42 (m, 2H), 3.35-3.32 (m, 2H), 2.71-2.64 (m, 2H), 2.55-2.51 (m, 3H), 2.47-2.40 (m, 2H), 2.15-2.08 (m, 2H), 1.96-1.79 (m, 4H), 1.51-1.37 (m, 2H). [M+H]⁺=833.70.

### Example 11: N-(3-carbamoyl-1-((1r,4r)-4-(4-(1-(4-((R)-2,6-dioxopiperidin-3-yl)-3,5-difluorophenyl)azetidine-3-carbonyl)piperazin-1-yl)cyclohexyl)-1H-pyrazol-4-yl)-2-(2-((2,2,2-trifluoroethyl)amino)pyridin-4-yl)oxazole-4-carboxamide

### Step 1: methyl 1-(4-(2,6-bis(benzyloxy)pyridin-3-yl)-3,5-difluorophenyl)azetidine-3-carboxylate

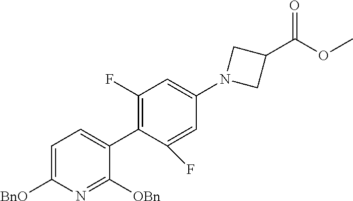 [SMILES: COC(=O)C1CN(c2cc(F)c(-c3ccc(OCc4ccccc4)nc3OCc3ccccc3)c(F)c2)C1]

A mixture of 2,6-bis(benzyloxy)-3-(4-bromo-2,6-difluorophenyl)pyridine (3.00 g, 6.22 mmol), methyl azetidine-3-carboxylate hydrochloride (1.41 g, 9.33 mmol), Cs₂CO₃ (6.06 g, 18.66 mmol) and RuPhos Pd G3 (520.7 mg, 0.622 mmol) in toluene (50 mL) was stirred overnight at 100° C. under nitrogen atmosphere. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EtOAc (2:1) to afford the product (1.7 g, 53%). [M+1]⁺=517.1.

### Step 2: 1-(4-(2,6-bis(benzyloxy)pyridin-3-yl)-3,5-difluorophenyl)azetidine-3-carboxylic acid

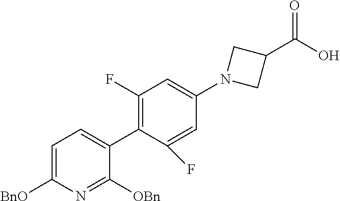 [SMILES: O=C(O)C1CN(c2cc(F)c(-c3ccc(OCc4ccccc4)nc3OCc3ccccc3)c(F)c2)C1]

To a stirred mixture of methyl 1-(4-(2,6-bis(benzyloxy)pyridin-3-yl)-3,5-difluorophenyl)azetidine-3-carboxylate (1.7 g, 3.29 mmol) in THF (20 mL) was added LiOH H₂O (168 mg, 4 mmol in 10 mL water) dropwise at room temperature. Then the mixture was stirred for 2 hours. The resulting mixture was concentrated in vacuum. The water layer was adjusted pH<5 with 1N HCl and then extracted with EtOAc (3×50 mL). The combined organic layers were washed with brine, dried over anhydrous Na₂SO₄. After filtration, the filtrate was concentrated under reduced pressure to afford the product (1.4 g, 85%) [M+1]⁺=503.2.

### Step 3: (R)-1-(4-(2,6-dioxopiperidin-3-yl)-3,5-difluorophenyl)azetidine-3-carboxylic acid

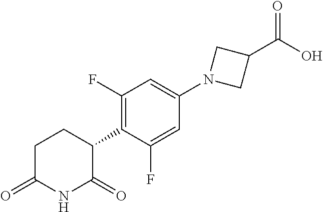 [SMILES: O=C1CC[C@H](c2c(F)cc(N3CC(C(=O)O)C3)cc2F)C(=O)N1]

To a solution of 1-(4-(2,6-bis(benzyloxy)pyridin-3-yl)-3,5-difluorophenyl)azetidine-3-carboxylic acid (1.40 g, 2.79 mmol) in iPrOH (20 mL) and DCM (20 mL) was added Pd/C (1.0 g, 10% wt) which was stirred at room temperature under hydrogen atmosphere for 48 hours. The resulting mixture was filtered, the filter cake was washed with MeOH (20 mL). The filtrate was concentrated under reduced pressure to afford the crude product. The residue was purified by SFC (IH (3*25 cm, 5 um), 13% EtOH/87% CO₂, 100 bar, 20 ml/min) and the title compound corresponded to peak A @1.853 min/254 nm (190 mg, 21%). [M+1]⁺=325.3.

### Step 4: N-(3-carbamoyl-1-((1r,4r)-4-(4-(1-(4-((R)-2,6-dioxopiperidin-3-yl)-3,5-difluorophenyl)azetidine-3-carbonyl)piperazin-1-yl)cyclohexyl)-1H-pyrazol-4-yl)-2-(2-((2,2,2-trifluoroethyl)amino)pyridin-4-yl)oxazole-4-carboxamide

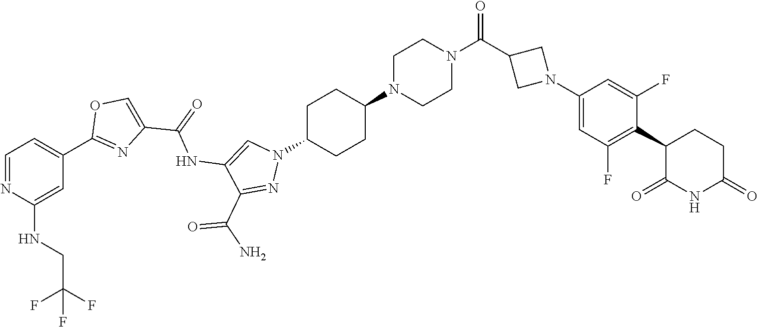 [SMILES: NC(=O)c1nn([C@H]2CC[C@H](N3CCN(C(=O)C4CN(c5cc(F)c([C@H]6CCC(=O)NC6=O)c(F)c5)C4)CC3)CC2)cc1NC(=O)c1coc(-c2ccnc(NCC(F)(F)F)c2)n1]

To a solution of N-(3-carbamoyl-1-((1r,4r)-4-(piperazin-1-yl)cyclohexyl)-1H-pyrazol-4-yl)-2-(2-((2,2,2-trifluoroethyl)amino)pyridin-4-yl)oxazole-4-carboxamide (56 mg, 0.1 mmol), (R)-1-(4-(2,6-dioxopiperidin-3-yl)-3,5-difluorophenyl)azetidine-3-carboxylic acid (32 mg, 0.1 mmol) and DIEA (65 mg, 0.5 mmol) in DMF (5 mL) was added HATU (38 mg, 0.1 mmol) at room temperature. The resulting mixture was stirred at room temperature for 18 hours. The reaction was evaporated under vacuum to afford the crude residue, which was purified with Prep-TLC (DCM:MeOH=8:1) to give product (36 mg, 42%). ¹H NMR (500 MHz, DMSO) δ 10.94 (s, 1H), 10.86 (s, 1H), 8.97 (s, 1H), 8.36 (s, 1H), 8.24 (d, J=5.5 Hz, 1H), 7.74-7.63 (m, 2H), 7.50 (s, 1H), 7.25 (s, 1H), 7.16 (d, J=5.5 Hz, 1H), 6.17 (d, J=11.0 Hz, 2H), 4.29-4.18 (m, 3H), 4.07-3.98 (m, 3H), 3.93-3.89 (m, 2H), 3.86-3.76 (m, 1H), 3.50-3.44 (m, 2H), 3.30-3.26 (m, 2H), 2.82-2.73 (m, 1H), 2.56-2.52 (m, 2H), 2.49-2.36 (m, 4H), 2.16-2.04 (m, 3H), 1.98-1.78 (m, 5H), 1.43 (dd, J=23.0, 11.5 Hz, 2H). [M+H]⁺=868.80.

### Example 9: N-(3-carbamoyl-1-((1r,4r)-4-(4-(3-(4-(((R)-2,6-dioxopiperidin-3-yl)amino)phenyl)cyclobutyl)piperazin-1-yl)cyclohexyl)-1H-pyrazol-4-yl)-2-(2-((2,2,2-trifluoroethyl)amino)pyridin-4-yl)oxazole-4-carboxamide

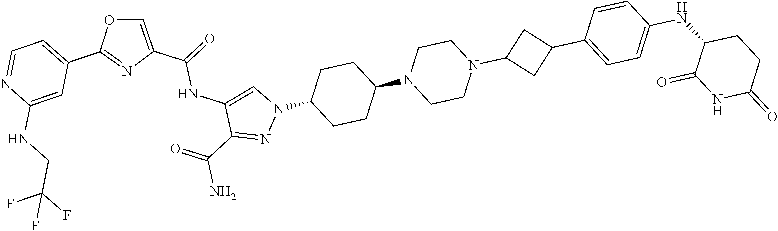 [SMILES: NC(=O)c1nn([C@H]2CC[C@H](N3CCN(C4CC(c5ccc(N[C@@H]6CCC(=O)NC6=O)cc5)C4)CC3)CC2)cc1NC(=O)c1coc(-c2ccnc(NCC(F)(F)F)c2)n1]

To a solution of N-(3-carbamoyl-1-((1r,4r)-4-(piperazin-1-yl)cyclohexyl)-1H-pyrazol-4-yl)-2-(2-((2,2,2-trifluoroethyl)amino)pyridin-4-yl)oxazole-4-carboxamide (56 mg, 0.1 mmol) and (R)-3-((4-(3-oxocyclobutyl)phenyl)amino)piperidine-2,6-dione (obtained through the similar way described in WO2021219070 A1) (30 mg, 0.11 mmol) in DCM/MeOH (5 mL/1 mL) was stirred at room temperature for 2 hours. The resulting mixture was added NaBH(OAc)₃ (106 mg, 0.5 mmol) and stirred at room temperature for 18 hours. The reaction was evaporated under vacuum to afford the crude residue, which was purified with Prep-HPLC to give product P1 (42 mg, 51.4% yield, trans:cis=86:12). ¹H NMR (500 MHz, DMSO) δ 10.94 (s, 1H), 10.76 (s, 1H), 8.97 (s, 1H), 8.36 (s, 1H), 8.24 (d, J=5.5 Hz, 1H), 8.19 (s, 1H), 7.72-7.62 (m, 2H), 7.49 (s, 1H), 7.25 (s, 1H), 7.15 (d, J=5.5 Hz, 1H), 7.03-6.91 (m, 2H), 6.61 (d, J=8.5 Hz, 2H), 5.67 (d, J=7.5 Hz, 1H), 4.30-4.18 (m, 4H), 2.99-2.90 (m, 1H), 2.78-2.70 (m, 1H), 2.67-2.58 (m, 2H), 2.58-2.52 (m, 4H), 2.42-2.25 (m, 6H), 2.15-2.07 (m, 3H), 1.99-1.91 (m, 2H), 1.89-1.81 (m, 3H), 1.79-1.69 (m, 2H), 1.42 (dd, J=23.0, 11.5 Hz, 2H). [M+H]⁺=818.70. And P2 (8 mg, 9.8% yield, trans:cis=30:57). ¹H NMR (500 MHz, DMSO) δ 10.94 (s, 1H), 10.77 (s, 1H), 8.97 (s, 1H), 8.36 (s, 1H), 8.24 (d, J=5.5 Hz, 1H), 8.17 (s, 1H), 7.72-7.61 (m, 2H), 7.49 (s, 1H), 7.25 (s, 1H), 7.16 (d, J=5.5 Hz, 1H), 7.01 (d, J=8.5 Hz, 1H), 6.95 (d, J=8.5 Hz, 1H), 6.62 (t, J=9.0 Hz, 2H), 5.67 (d, J=7.5 Hz, 1H), 4.31-4.17 (m, 4H), 3.01-2.69 (m, 2H), 2.67-2.52 (m, 6H), 2.43-2.30 (m, 4H), 2.29-2.19 (m, 2H), 2.15-2.07 (m, 3H), 2.06-2.01 (m, 1H), 1.99-1.91 (m, 2H), 1.90-1.79 (m, 3H), 1.78-1.63 (m, 1H), 1.49-1.33 (m, 2H). [M+H]⁺=818.70.

### Example 10: N-(3-carbamoyl-1-((1r,4r)-4-(4-(3-(4-(((S)-2,6-dioxopiperidin-3-yl)amino)phenyl)cyclobutyl)piperazin-1-yl)cyclohexyl)-1H-pyrazol-4-yl)-2-(2-((2,2,2-trifluoroethyl)amino)pyridin-4-yl)oxazole-4-carboxamide

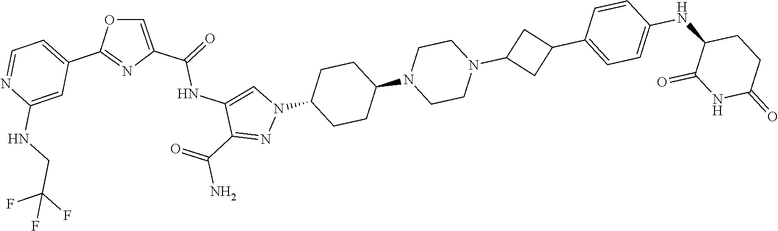 [SMILES: NC(=O)c1nn([C@H]2CC[C@H](N3CCN(C4CC(c5ccc(N[C@H]6CCC(=O)NC6=O)cc5)C4)CC3)CC2)cc1NC(=O)c1coc(-c2ccnc(NCC(F)(F)F)c2)n1]

To a solution of N-(3-carbamoyl-1-((1r,4r)-4-(piperazin-1-yl)cyclohexyl)-1H-pyrazol-4-yl)-2-(2-((2,2,2-trifluoroethyl)amino)pyridin-4-yl)oxazole-4-carboxamide (56 mg, 0.1 mmol) and (S)-3-((4-(3-oxocyclobutyl)phenyl)amino)piperidine-2,6-dione (obtained through the similar way described in WO2021219070 A1) (30 mg, 0.11 mmol) in DCM/MeOH (5 mL/1 mL) was stirred at room temperature for 2 hours. The resulting mixture was added NaBH(OAc)₃ (106 mg, 0.5 mmol) and stirred at room temperature for 18 hours. The reaction was evaporated under vacuum to afford the crude residue, which was purified with Prep-HPLC to afford product (16 mg, 20% yield, trans:cis=72:18). ¹H NMR (500 MHz, DMSO) δ 10.95 (s, 1H), 10.79 (s, 1H), 8.98 (s, 1H), 8.39 (s, 1H), 8.25 (d, J=5.5 Hz, 1H), 7.72 (t, J=6.0 Hz, 1H), 7.60 (s, 1H), 7.57 (s, 1H), 7.25 (s, 1H), 7.16 (d, J=5.5 Hz, 1H), 7.08-6.98 (m, 2H), 6.69-6.59 (m, 2H), 4.36-4.18 (m, 4H), 3.66-3.22 (m, 5H), 3.06-3.01 (m, 1H), 2.83-2.67 (m, 2H), 2.65-2.51 (m, 5H), 2.49-2.43 (m, 3H), 2.32-2.17 (m, 3H), 2.16-2.04 (m, 4H), 1.96-1.83 (m, 3H), 1.69-1.51 (m, 2H). [M+H]⁺=818.60.

### Example 76: N-(3-(difluoromethyl)-1-((1r,4r)-4-(4-(1-(4-(2,4-dioxotetrahydropyrimidin-1(2H)-yl)-3,5-difluorophenyl)azetidine-3-carbonyl)piperazin-1-yl)cyclohexyl)-1H-pyrazol-4-yl)-2-(2-((2,2,2-trifluoroethyl)amino)pyridin-4-yl)oxazole-4-carboxamide

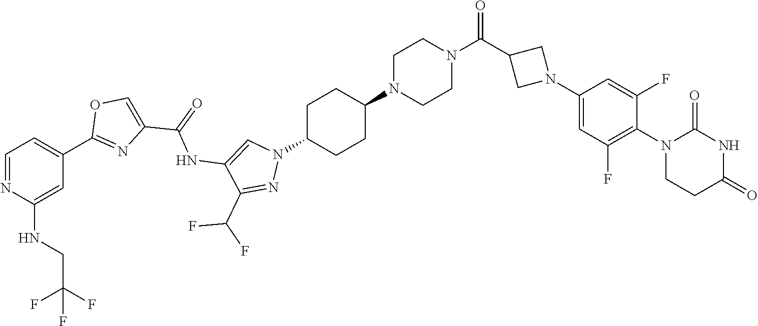 [SMILES: O=C1CCN(c2c(F)cc(N3CC(C(=O)N4CCN([C@H]5CC[C@H](n6cc(NC(=O)c7coc(-c8ccnc(NCC(F)(F)F)c8)n7)c(C(F)F)n6)CC5)CC4)C3)cc2F)C(=O)N1]

The title compound was prepared in a manner similar to that described in Example 13.

¹H NMR (500 MHz, DMSO) δ 10.50 (s, 1H), 9.71 (s, 1H), 8.95 (s, 1H), 8.24 (d, J=5.0 Hz, 1H), 8.18 (s, 1H), 7.62-7.60 (m, 1H), 7.24-7.05 (m, 3H), 6.25 (d, J=10.0 Hz, 2H), 4.26-4.19 (m, 3H), 4.07-4.04 (m, 2H), 3.96-3.93 (m, 2H), 3.85-3.82 (m, 1H), 3.59 (t, J=5.0 Hz, 2H), 3.48 (s, 2H), 3.34 (s, 2H), 2.68 (t, J=5.0 Hz, 2H), 2.48-2.43 (m, 5H), 2.10-2.08 (m, 2H), 1.92-1.89 (m, 2H), 1.81-1.76 (m, 2H), 1.47-1.42 (m, 2H). [M+H]⁺=876.7.

### Example 100: 2-(2-((cyclopropylmethyl)amino)pyridin-4-yl)-N-(3-(difluoromethyl)-1-((1r,4r)-4-(4-(1-(4-((R)-2,6-dioxopiperidin-3-yl)-3,5-difluorophenyl)azetidine-3-carbonyl)-2-oxopiperazin-1-yl)cyclohexyl)-1H-pyrazol-4-yl)oxazole-4-carboxamide

### Step 1: tert-butyl 4-((1r,4r)-4-(4-(2-(2-((cyclopropylmethyl)amino)pyridin-4-yl)oxazole-4-carboxamido)-3-(difluoromethyl)-1H-pyrazol-1-yl)cyclohexyl)-3-oxopiperazine-1-carboxylate

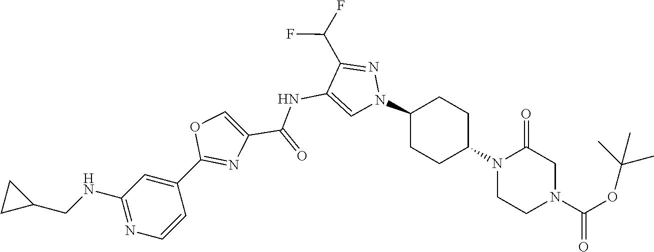 [SMILES: CC(C)(C)OC(=O)N1CCN([C@H]2CC[C@H](n3cc(NC(=O)c4coc(-c5ccnc(NCC6CC6)c5)n4)c(C(F)F)n3)CC2)C(=O)C1]

To a solution of tert-butyl 4-((1r,4r)-4-(4-(2-(2-((cyclopropylmethyl)amino)pyridin-4-yl)oxazole-4-carboxamido)-3-(difluoromethyl)-1H-pyrazol-1-yl)cyclohexyl)piperazine-1-carboxylate which was synthesize in a manner method to Example 13 (200 mg, 0.312 mmol) in THF (10 mL) and H₂O (4 mL) was added I₂ (594 mg, 2.34 mmol) and NaHCO₃ (262 mg, 3.12 mmol). The mixture was stirred overnight at room temperature under N₂. The reaction was quenched with Na₂S₂O₃ aq. (20 mL) and extracted with EA (30 mL×3). The organic layer was dried over with Na₂SO₄, filtered and concentrated to afford the product (150 mg, 73%). [M+H]⁺=655.3.

### Step 2: 2-(2-((cyclopropylmethyl)amino)pyridin-4-yl)-N-(3-(difluoromethyl)-1-((1r,4r)-4-(2-oxopiperazin-1-yl)cyclohexyl)-1H-pyrazol-4-yl)oxazole-4-carboxamide

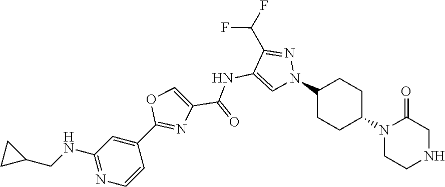

To a DCM solution (10 mL) of tert-butyl 4-((1r,4r)-4-(4-(2-(2-((cyclopropylmethyl)amino)pyridin-4-yl)oxazole-4-carboxamido)-3-(difluoromethyl)-1H-pyrazol-1-yl)cyclohexyl)-3-oxopiperazine-1-carboxylate (20 mg, 0.23 mmol) was added TFA (5 mL). The mixture was stirred overnight at room temperature. The solvent was removed under vacuum. The residue was basified to pH>7 with Na₂CO₃ aq., extracted with DCM (50 mL×3). The organic layer was dried over Na₂SO₄, filtered and concentrated to afford crude product (100 mg, 78%). [M+H]⁺=555.3

### Step 3: 2-(2-((cyclopropylmethyl)amino)pyridin-4-yl)-N-(3-(difluoromethyl)-1-((1r,4r)-4-(4-(1-(4-((R)-2,6-dioxopiperidin-3-yl)-3,5-difluorophenyl)azetidine-3-carbonyl)-2-oxopiperazin-1-yl)cyclohexyl)-1H-pyrazol-4-yl)oxazole-4-carboxamide

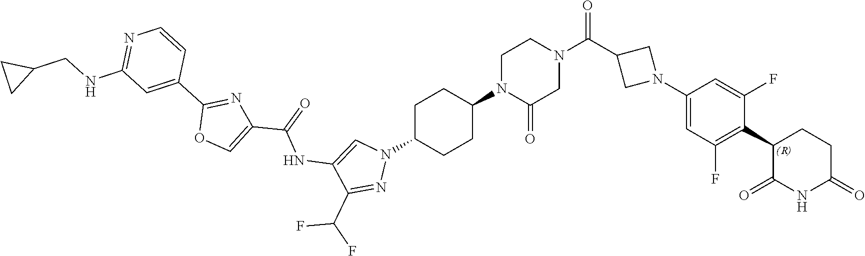

To a DMF solution (5 mL) of 2-(2-((cyclopropylmethyl)amino)pyridin-4-yl)-N-(3-(difluoromethyl)-1-((1r,4r)-4-(2-oxopiperazin-1-yl)cyclohexyl)-1H-pyrazol-4-yl)oxazole-4-carboxamide (50 mg, 0.09 mmol) was added (R)-1-(4-(2,6-dioxopiperidin-3-yl)-3,5-difluorophenyl)azetidine-3-carboxylic acid (35 mg, 0.108 mmol), DIEA (0.5 mL) and HATU (51 mg, 0.135 mmol). The mixture was stirred overnight at room temperature. The mixture was purified with combi flash, eluted with DCM:MeOH=1-0 to 1:10 to yield the crude product which was further purified with prep. HPLC to afford the product (13 mg, 17%). ¹H NMR (500 MHz, DMSO) δ 10.86 (s, 1H), 9.73 (s, 1H), 8.93 (s, 1H), 8.22 (s, 1H), 8.15 (d, J=5.0 Hz, 1H), 7.27-7.01 (m, 4H), 6.17 (d, J=10.0 Hz, 2H), 4.54-4.52 (m, 1H), 4.36-4.26 (m, 2H), 4.04-4.02 (m, 5H), 3.94-3.90 (m, 3H), 3.68-3.57 (m, 2H), 3.18 (t, J=5.0 Hz, 2H), 2.81-2.74 (m, 1H), 2.54-2.51 (m, 3H), 2.13-2.04 (m, 3H), 1.94-1.89 (m, 3H), 1.79-1.72 (m, 4H), 1.12-1.08 (m, 1H), 0.46-0.45 (m, 2H), 0.23-0.22 (m, 2H). [M+H]⁺=861.6.

### Example 101: 2-(2-((cyclopropylmethyl)amino)pyridin-4-yl)-N-(3-(difluoromethyl)-1-((1R,4r)-4-(4-((R)-1-(4-((R)-2,6-dioxopiperidin-3-yl)-3,5-difluorophenyl)pyrrolidine-3-carbonyl)-2-oxopiperazin-1-yl)cyclohexyl)-1H-pyrazol-4-yl)oxazole-4-carboxamide

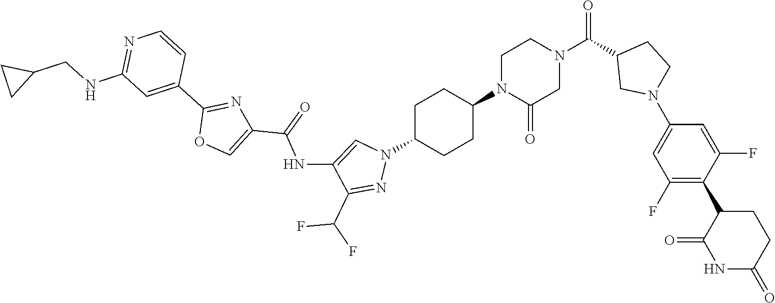

The title compound was prepared in a manner similar to that described in Example 100.

¹H NMR (500 MHz, DMSO) δ 10.84 (s, 1H), 9.72 (s, 1H), 8.93 (s, 1H), 8.22 (s, 1H), 8.15 (d, J=5.0 Hz, 1H), 7.27-7.01 (m, 4H), 6.23 (d, J=10.0 Hz, 2H), 4.37-4.26 (m, 3H), 4.04-4.00 (m, 2H), 3.81-3.80 (m, 1H), 3.68-3.67 (m, 1H), 3.56-3.53 (m, 1H), 3.47-3.39 (m, 3H), 3.29-3.19 (m, 2H), 3.18 (d, J=5.0 Hz, 2H), 2.81-2.74 (m, 1H), 2.55 (s, 2H), 2.19-2.05 (m, 5H), 1.94-1.88 (m, 3H), 1.78-1.73 (m, 4H), 1.47-1.40 (m, 2H), 1.09-1.04 (m, 1H), 0.46-0.45 (m, 2H), 0.23-0.22 (m, 2H). [M+H]⁺=875.8.

### Example 166: N-(3-carbamoyl-1-(6-(4-((R)-1-(4-((R)-2,6-dioxopiperidin-3-yl)-3,5-difluorophenyl)pyrrolidine-3-carbonyl)piperazin-1-yl)pyridin-3-yl)-1H-pyrazol-4-yl)-2-(2-((2,2,2-trifluoroethyl)amino)pyridin-4-yl)oxazole-4-carboxamide

### Step 1: (6-(4-(tert-butoxycarbonyl)piperazin-1-yl)pyridin-3-yl)boronic acid

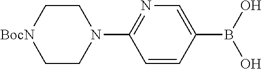 [SMILES: CC(C)(C)OC(=O)N1CCN(c2ccc(B(O)O)cn2)CC1]

A mixture of tert-butyl 4-(5-(4,4,5,5-tetramethyl-1,3,2-dioxaborolan-2-yl)pyridin-2-yl)piperazine-1-carboxylate (1 g, 2.57 mmol), NaIO₄ (2.75 g, 12.85 mmol) and NH₄OAc (989 mg, 12.85 mmol) in tetrahydrofuran (10 mL) and water (10 mL) was stirred in a round bottom flask at room temperature for overnight. The resulting mixture was extracted with DCM (3×10 mL) and water (20 mL). The combined organic layer was washed with brine (20 mL), dried over anhydrous Na₂SO₄, filtered, and concentrated under reduced pressure to yield the title product (780 mg, 99%). [M+H]⁺=308.

### Step 2: tert-butyl 4-(5-(3-(ethoxycarbonyl)-4-nitro-1H-pyrazol-1-yl)pyridin-2-yl)piperazine-1-carboxylate

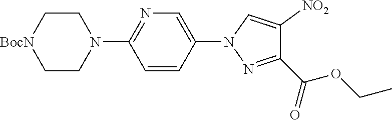 [SMILES: CCOC(=O)c1nn(-c2ccc(N3CCN(C(=O)OC(C)(C)C)CC3)nc2)cc1[N+](=O)[O-]]

A mixture of (6-(4-(tert-butoxycarbonyl)piperazin-1-yl)pyridin-3-yl)boronic acid (780 mg, 2.54 mmol), ethyl 4-nitro-1H-pyrazole-3-carboxylate (235 mg, 1.27 mmol), pyridine (200 mg, 2.54 mmol) and cupric acetate (346.7 mg, 1.9 mmol) in DCM (20.0 mL) was stirred room temperature for 16 h under oxygen. The resulting mixture was extracted with DCM (3×20 mL) and washed with water (20 mL). The combined organic layer was washed with brine (20 mL), dried over anhydrous Na₂SO₄, filtered, and concentrated under reduced pressure to afford the crude product, which was purified with silica gel column chromatography (PE:EA=5: 1-1:2 gradient elution) to afford the product (250 mg, 44%). [M+H]⁺=447.2.

### Step 3: tert-butyl 4-(5-(3-carbamoyl-4-nitro-1H-pyrazol-1-yl)pyridin-2-yl)piperazine-1-carboxylate

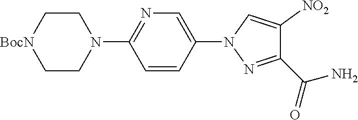 [SMILES: CC(C)(C)OC(=O)N1CCN(c2ccc(-n3cc([N+](=O)[O-])c(C(N)=O)n3)cn2)CC1]

A mixture of tert-butyl 4-(5-(3-(ethoxycarbonyl)-4-nitro-1H-pyrazol-1-yl)pyridin-2-yl)piperazine-1-carboxylate (250 mg, 0.56 mmol) and ammonium hydroxide (10 mL) in THF (10.0 mL) was stirred at 80° C. for 15 h in sealing tube. The resulting solution was extracted with DCM (2×20.0 mL) and washed with water (20 mL). The organic layer was dried over anhydrous sodium sulfate and concentrated under vacuum to yield the product (194 mg). [M−55]+=362.2.

### Step 4: tert-butyl 4-(5-(4-amino-3-carbamoyl-1H-pyrazol-1-yl)pyridin-2-yl)piperazine-1-carboxylate

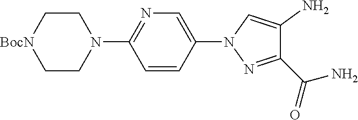 [SMILES: CC(C)(C)OC(=O)N1CCN(c2ccc(-n3cc(N)c(C(N)=O)n3)cn2)CC1]

A solution of tert-butyl 4-(5-(3-carbamoyl-4-nitro-1H-pyrazol-1-yl)pyridin-2-yl)piperazine-1-carboxylate (194 mg, 0.465 mmol) in THF (15.0 mL) was added 10% wt Pd/C (20.0 mg) at 25° C. Then the flask was evacuated and backfilled with H₂ for two times and stirred under H₂ atmosphere at 25° C. for 15 h. Reaction was monitored by LCMS. The mixture was filtered through a pad of Celite and the filter cake was washed with MeOH (20.0 mL). The filtrate was concentrated under vacuum to obtain the product (188.0 mg). [M+H]⁺=388.2.

### Step 5: tert-butyl 4-(5-(4-(2-(2-((tert-butoxycarbonyl)(2,2,2-trifluoroethyl)amino)pyridin-4-yl)oxazole-4-carboxamido)-3-carbamoyl-1H-pyrazol-1-yl)pyridin-2-yl)piperazine-1-carboxylate

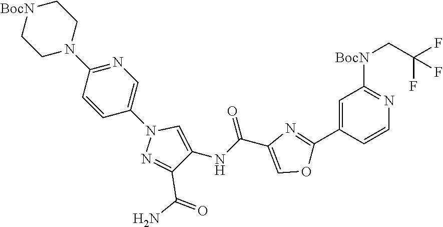 [SMILES: CC(C)(C)OC(=O)N1CCN(c2ccc(-n3cc(NC(=O)c4coc(-c5ccnc(N(CC(F)(F)F)C(=O)OC(C)(C)C)c5)n4)c(C(N)=O)n3)cn2)CC1]

A mixture of 2-(2-((tert-butoxycarbonyl)(2,2,2-trifluoroethyl)amino)pyridin-4-yl)oxazole-4-carboxylic acid (188.0 mg, 0.486 mmol), HATU (277.0 mg, 0.728 mmol), and DIEA (188.0 mg, 1.457 mmol) in DMF (20 mL) was stirred at room temperature for 40 minute, and tert-butyl 4-(5-(4-amino-3-carbamoyl-1H-pyrazol-1-yl)pyridin-2-yl)piperazine-1-carboxylate (188.0 mg, 0.486 mmol) was added. The mixture was stirred at room temperature for 16 h. Reaction was monitored with LCMS. Water (80 mL) was added and the solid was collected by filtration, washed with water (50.0 mL), and dried to obtain the product (360 mg, 98%). [M−99]⁺=657.6.

### Step 6: N-(3-carbamoyl-1-(6-(piperazin-1-yl)pyridin-3-yl)-1H-pyrazol-4-yl)-2-(2-((2,2,2-trifluoroethyl)amino)pyridin-4-yl)oxazole-4-carboxamide hydrochloride

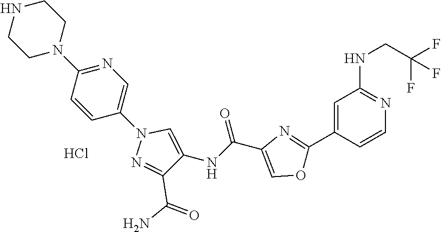 [SMILES: NC(=O)c1nn(-c2ccc(N3CCNCC3)nc2)cc1NC(=O)c1coc(-c2ccnc(NCC(F)(F)F)c2)n1 ; Cl]

tert-butyl 4-(5-(4-(2-(2-((tert-butoxycarbonyl)(2,2,2-trifluoroethyl)amino)pyridin-4-yl)oxazole-4-carboxamido)-3-carbamoyl-1H-pyrazol-1-yl)pyridin-2-yl)piperazine-1-carboxylate (360 mg, 0.476 mmol) in DCM (10 mL) was added 4N HCl/dioxane (15 mL) at 25° C. The mixture was stirred at 25° C. for 1 h. Reaction was monitored with LCMS. The solid was collected by filtration, washed with DCM (20.0 mL), and dried to obtain the product (260 mg, 93%). [M+H]⁺=557.3.

### Step 7: N-(3-carbamoyl-1-(6-(4-((R)-1-(4-((R)-2,6-dioxopiperidin-3-yl)-3,5-difluorophenyl)pyrrolidine-3-carbonyl)piperazin-1-yl)pyridin-3-yl)-1H-pyrazol-4-yl)-2-(2-((2,2,2-trifluoroethyl)amino)pyridin-4-yl)oxazole-4-carboxamide

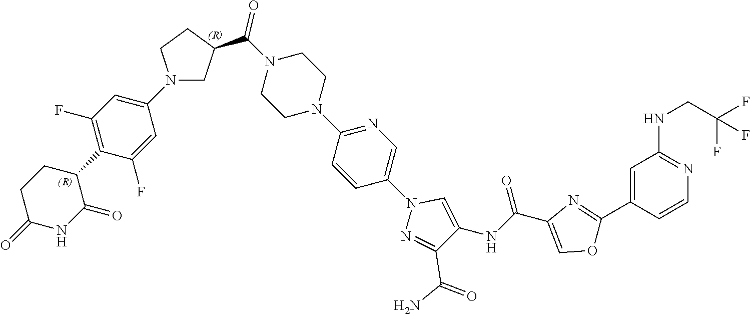

A mixture of N-(3-carbamoyl-1-(6-(piperazin-1-yl)pyridin-3-yl)-1H-pyrazol-4-yl)-2-(2-((2,2,2-trifluoroethyl)amino)pyridin-4-yl)oxazole-4-carboxamide hydrochloride (30.0 mg, 0.051 mmol), (R)-1-(4-((R)-2,6-dioxopiperidin-3-yl)-3,5-difluorophenyl)pyrrolidine-3-carboxylic acid (19.0 mg, 0.056 mmol), T₃P (1 mL) and DIEA (20 mg, 0.152 mmol) in DCM (8 mL) was stirred at room temperature for 16 h. Reaction was monitored with LCMS. The resulting solution was extracted with DCM (2×15.0 mL) and washed with water (20 mL). The organic layer was dried over anhydrous sodium sulfate and concentrated under vacuum to afford the crude product. The crude product was purified with Prep-HPLC to afford the product (4 mg, 9%). ¹H NMR (500 MHz, DMSO) δ 11.01 (s, 1H), 10.84 (s, 1H), 9.01 (s, 1H), 8.85 (s, 1H), 8.71 (d, J=2.5 Hz, 1H), 8.26 (d, J=5.2 Hz, 1H), 8.13 (dd, J=9.1, 2.5 Hz, 1H), 8.00 (s, 1H), 7.69 (s, 2H), 7.27 (s, 1H), 7.18 (d, J=5.2 Hz, 1H), 7.05 (d, J=9.2 Hz, 1H), 6.24 (d, J=12.2 Hz, 2H), 4.30-4.18 (m, 2H), 4.02 (dd, J=12.4, 4.9 Hz, 1H), 3.79-3.53 (m, 10H), 3.49 (t, J=8.8 Hz, 1H), 3.37-3.42 (m, 1H), 3.25-3.28 (m, 2H), 2.82-2.74 (m, 1H), 2.26-2.05 (m, 3H), 1.99-1.91 (m, 1H); [M+H]⁺=877.6.

### Example 30: N-(3-carbamoyl-1-(6-(4-((R)-1-(4-((R)-2,6-dioxopiperidin-3-yl)-3,5-difluorophenyl)pyrrolidine-3-carbonyl)piperazin-1-yl)pyridin-3-yl)-1H-pyrazol-4-yl)-2-(2-methylpyridin-4-yl)oxazole-4-carboxamide

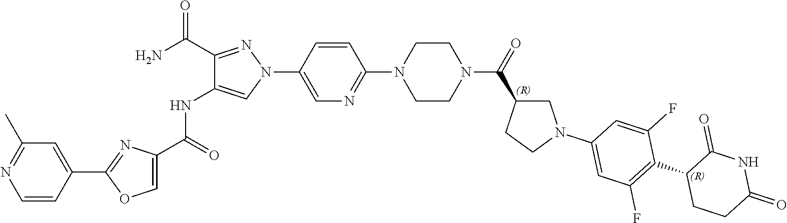

The title compound was prepared in a manner similar to that described in Example 166.

¹H NMR (500 MHz, DMSO) δ 11.02 (s, 1H), 10.77 (s, 1H), 9.00 (s, 1H), 8.78 (s, 1H), 8.64 (d, J=4.6 Hz, 2H), 8.07 (dd, J=9.1, 2.7 Hz, 1H), 7.94 (s, 1H), 7.76 (s, 1H), 7.69 (d, J=5.9 Hz, 2H), 6.98 (d, J=9.3 Hz, 1H), 6.18 (d, J=12.1 Hz, 2H), 3.95 (dd, J=12.6, 5.1 Hz, 1H), 3.58 (dt, J=15.0, 12.3 Hz, 9H), 3.45-3.30 (m, 3H), 3.24-3.08 (m, 2H), 2.76-2.67 (m, 1H), 2.55 (s, 3H), 2.18-1.96 (m, 3H), 1.92-1.84 (m, 1H). [M+H]⁺=794.6.

### Example 31: N-(3-carbamoyl-1-(6-(4-(1-(4-(2,4-dioxotetrahydropyrimidin-1(2H)-yl)phenyl)azetidine-3-carbonyl)piperazin-1-yl)pyridin-3-yl)-1H-pyrazol-4-yl)-2-(2-methylpyridin-4-yl)oxazole-4-carboxamide

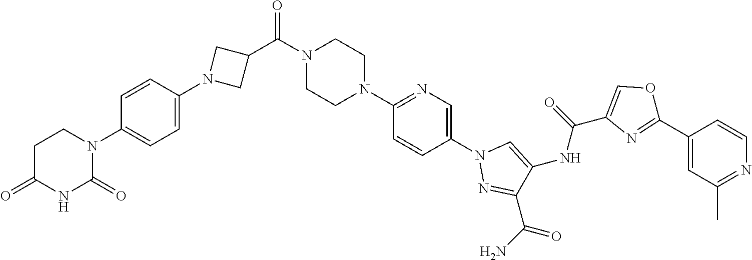 [SMILES: Cc1cc(-c2nc(C(=O)Nc3cn(-c4ccc(N5CCN(C(=O)C6CN(c7ccc(N8CCC(=O)NC8=O)cc7)C6)CC5)nc4)nc3C(N)=O)co2)ccn1]

The title compound was prepared in a manner similar to that described in Example 166. ¹H NMR (500 MHz, DMSO) δ 11.10 (s, 1H), 10.27 (s, 1H), 9.10 (s, 1H), 8.86 (s, 1H), 8.72 (dd, J=9.3, 3.8 Hz, 2H), 8.15 (d, J=9.1 Hz, 1H), 8.05 (s, 1H), 7.88 (s, 1H), 7.81 (d, J=4.6 Hz, 1H), 7.77 (s, 1H), 7.12 (d, J=8.5 Hz, 2H), 7.05 (d, J=9.2 Hz, 1H), 6.47 (d, J=8.6 Hz, 2H), 4.09-4.02 (m, 2H), 3.95-3.87 (s, 3H), 3.67 (t, J=6.7 Hz, 2H), 3.65-3.58 (m, 6H), 3.53-3.47 (m, 2H), 2.68 (t, J=6.6 Hz, 2H), 2.63 (s, 3H). [M+H]⁺=745.6.

### Example 51: N-(3-(difluoromethyl)-1-((1r,4r)-4-(4-(1-(4-((R)-2,6-dioxopiperidin-3-yl)-3,5-difluorophenyl)azetidine-3-carbonyl)piperazin-1-yl)cyclohexyl)-1H-pyrazol-4-yl)-2-(2-((2,2,2-trifluoroethyl)amino)pyridin-4-yl)-2H-1,2,3-triazole-4-carboxamide

### Step 1: 2-(2-((tert-butoxycarbonyl)(2,2,2-trifluoroethyl)amino)pyridin-4-yl)-2H-1,2,3-triazole-4-carboxylic acid

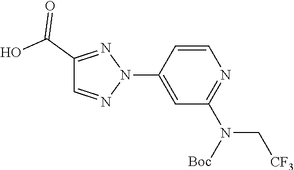 [SMILES: CC(C)(C)OC(=O)N(CC(F)(F)F)c1cc(-n2ncc(C(=O)O)n2)ccn1]

To a DMF solution (30 mL) of tert-butyl (4-bromopyridin-2-yl)(2,2,2-trifluoroethyl)carbamate (3.1 g, 8.69 mmol) was added methyl 2H-1,2,3-triazole-4-carboxylate (1.0 g, 7.9 mmol), Fe(acac)₃ (837 mg, 2.37 mmol), CuO (63 mg, 0.79 mmol) and Cs₂CO₃ (5.2 g, 15.8 mmol). The mixture was stirred for 16 h at 100° C. under N₂. The solid was filtered and the filtrate was purified with preparative HPLC to afford the product (2.5 g, 74%). [M+H]⁺=388.1.

### Step 2: tert-butyl 4-((1r,4r)-4-(4-(2-(2-((tert-butoxycarbonyl)(2,2,2-trifluoroethyl)amino)pyridin-4-yl)-2H-1,2,3-triazole-4-carboxamido)-3-(difluoromethyl)-1H-pyrazol-1-yl)cyclohexyl)piperazine-1-carboxylate

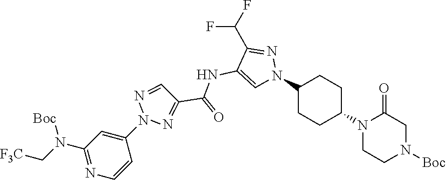

To a DMF solution (5 mL) of tert-butyl 4-((trans)-4-(4-amino-3-(difluoromethyl)-1H-pyrazol-1-yl)cyclohexyl)piperazine-1-carboxylate (300 mg, 0.752 mmol) in was added 2-(2-((tert-butoxycarbonyl)(2,2,2-trifluoroethyl)amino)pyridin-4-yl)-2H-1,2,3-triazole-4-carboxylic acid (291 mg, 0.752 mmol), DIEA (1.0 mL) and HATU (429 mg, 1.128 mmol). The mixture was stirred overnight at room temperature. The mixture was purified with combi flash, eluted with DCM:MeOH=1-0 to 1:10 to afford the product (300 mg, 52%). [M+H]⁺=769.4.

### Step 3: N-(3-(difluoromethyl)-1-((1r,4r)-4-(piperazin-1-yl)cyclohexyl)-1H-pyrazol-4-yl)-2-(2-((2,2,2-trifluoroethyl)amino)pyridin-4-yl)-2H-1,2,3-triazole-4-carboxamide

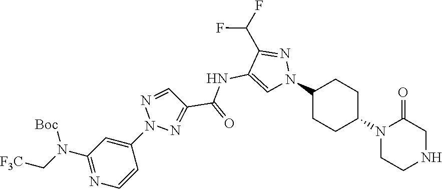

To a DCM solution (10 mL) of tert-butyl 4-((1r,4r)-4-(4-(2-(2-((tert-butoxycarbonyl)(2,2,2-trifluoroethyl)amino)pyridin-4-yl)-2H-1,2,3-triazole-4-carboxamido)-3-(difluoromethyl)-1H-pyrazol-1-yl)cyclohexyl)piperazine-1-carboxylate (800 mg, 1.04 mmol) was added TFA (10 mL). The mixture was stirred overnight at room temperature. The solvent was removed under vacuum. The residue was basified to pH>7 with Na₂CO₃ aq., extracted with DCM (50 mL×3). The organic layer was dried over Na₂SO₄, filtered and concentrated to afford crude product (580 mg, 98%). [M+H]⁺=569.2.

### Step 4: N-(3-(difluoromethyl)-1-((1r,4r)-4-(4-(1-(4-((R)-2,6-dioxopiperidin-3-yl)-3,5-difluorophenyl)azetidine-3-carbonyl)piperazin-1-yl)cyclohexyl)-1H-pyrazol-4-yl)-2-(2-((2,2,2-trifluoroethyl)amino)pyridin-4-yl)-2H-1,2,3-triazole-4-carboxamide

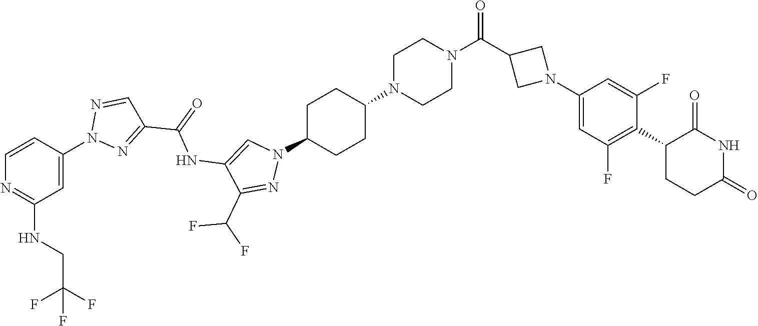 [SMILES: O=C1CC[C@H](c2c(F)cc(N3CC(C(=O)N4CCN([C@H]5CC[C@H](n6cc(NC(=O)c7cnn(-c8ccnc(NCC(F)(F)F)c8)n7)c(C(F)F)n6)CC5)CC4)C3)cc2F)C(=O)N1]

To a DCM solution (3 mL) of N-(3-(difluoromethyl)-1-((1r,4r)-4-(piperazin-1-yl)cyclohexyl)-1H-pyrazol-4-yl)-2-(2-((2,2,2-trifluoroethyl)amino)pyridin-4-yl)-2H-1,2,3-triazole-4-carboxamide (10 mg, 0.018 mmol) was added (R)-1-(4-(2,6-dioxopiperidin-3-yl)-3,5-difluorophenyl)azetidine-3-carboxylic acid (7.0 mg, 0.0216 mmol), DIEA (0.5 mL) and HATU (14 mg, 0.036 mmol). The mixture was stirred overnight at room temperature. The mixture was purified with combi flash, eluted with DCM:MeOH=1-0 to 1:10 to afford the crude product which was further purified with prepHPLC to yield the product (12 mg, 76%).

¹H NMR (500 MHz, DMSO) δ 10.86 (s, 1H), 10.24 (s, 1H), 8.63 (s, 1H), 8.25 (d, J=5.0 Hz, 1H), 8.15 (s, 1H), 7.70-7.67 (m, 1H), 7.33-7.32 (m, 2H), 7.24-7.03 (m, 1H), 6.17 (d, J=10.0 Hz, 2H), 4.28-4.20 (m, 3H), 4.04-4.01 (m, 3H), 3.92-3.90 (m, 2H), 3.84-3.81 (m, 1H), 3.47 (s, 2H), 2.78-2.74 (m, 1H), 2.54-2.41 (m, 8H), 2.11-2.07 (m, 3H), 1.94-1.90 (m, 3H), 1.84-1.77 (m, 2H), 1.47-1.42 (m, 2H). [M+H]⁺=875.8.

### Example 52: N-(3-(difluoromethyl)-1-((1R,4r)-4-(4-((R)-1-(4-((R)-2,6-dioxopiperidin-3-yl)-3,5-difluorophenyl)pyrrolidine-3-carbonyl)piperazin-1-yl)cyclohexyl)-1H-pyrazol-4-yl)-2-(2-((2,2,2-trifluoroethyl)amino)pyridin-4-yl)-2H-1,2,3-triazole-4-carboxamide

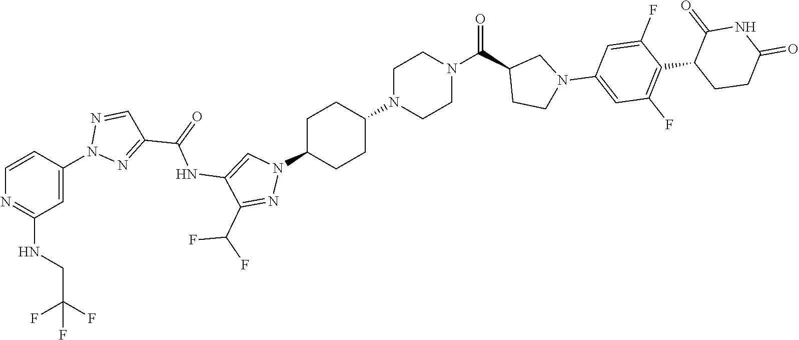 [SMILES: O=C1CC[C@H](c2c(F)cc(N3CC[C@@H](C(=O)N4CCN([C@H]5CC[C@H](n6cc(NC(=O)c7cnn(-c8ccnc(NCC(F)(F)F)c8)n7)c(C(F)F)n6)CC5)CC4)C3)cc2F)C(=O)N1]

The title compound was prepared in a manner similar to that described in Example 51.

¹H NMR (500 MHz, DMSO) δ 10.77 (s, 1H), 10.18 (s, 1H), 8.56 (s, 1H), 8.18-8.06 (m, 2H), 7.62-7.61 (m, 1H), 7.27-6.96 (m, 3H), 6.16 (d, J=10.0 Hz, 2H), 4.19-4.17 (m, 3H), 3.97-3.94 (m, 1H), 3.47-3.37 (m, 7H), 2.71-2.68 (m, 1H), 2.54-2.41 (m, 8H), 2.06-2.02 (m, 5H), 1.86-1.73 (m, 5H), 1.38-1.37 (m, 2H). [M+H]⁺=889.8.

### Example 61: 2-(2-((cyclopropylmethyl)amino)pyridin-4-yl)-N-(3-(difluoromethyl)-1-((1r,4r)-4-(4-(1-(4-(2,4-dioxotetrahydropyrimidin-1(2H)-yl)-3,5-difluorophenyl)azetidine-3-carbonyl)piperazin-1-yl)cyclohexyl)-1H-pyrazol-4-yl)-2H-1,2,3-triazole-4-carboxamide

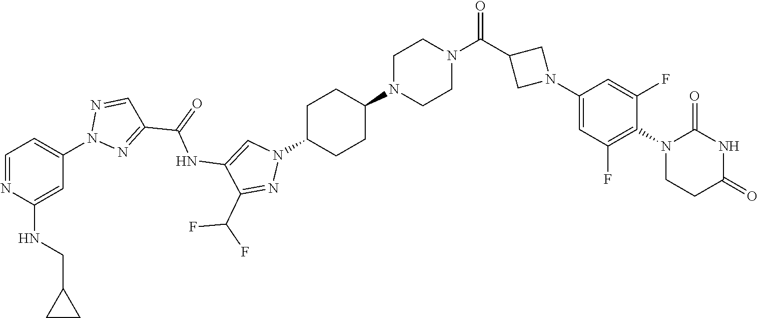

The title compound was prepared in a manner similar to that described in Example 51.

¹H NMR (500 MHz, DMSO) δ 10.50 (s, 1H), 10.22 (s, 1H), 8.60 (s, 1H), 8.16-8.14 (m, 2H), 7.24-7.03 (m, 4H), 6.25 (d, J=10.0 Hz, 2H), 4.24-4.20 (m, 1H), 4.07-4.04 (m, 2H), 3.96-3.93 (m, 2H), 3.87-3.82 (m, 1H), 3.59 (t, J=5.0 Hz, 2H), 3.51-3.45 (m, 3H), 3.20 (t, J=5.0 Hz, 2H), 2.68 (t, J=5.0 Hz, 2H), 2.54-2.41 (m, 6H), 2.11-2.09 (m, 2H), 1.93-1.77 (m, 4H), 1.48-1.41 (m, 2H), 1.07-1.06 (m, 1H), 0.46-0.45 (m, 2H), 0.23-0.22 (m, 2H). [M+H]⁺=848.6.

### Example 63: 2-(2-((cyclopropylmethyl)amino)pyridin-4-yl)-N-(3-(difluoromethyl)-1-((1S,4r)-4-(4-((S)-1-(4-(2,4-dioxotetrahydropyrimidin-1(2H)-yl)-3,5-difluorophenyl)pyrrolidine-3-carbonyl)piperazin-1-yl)cyclohexyl)-1H-pyrazol-4-yl)-2H-1,2,3-triazole-4-carboxamide

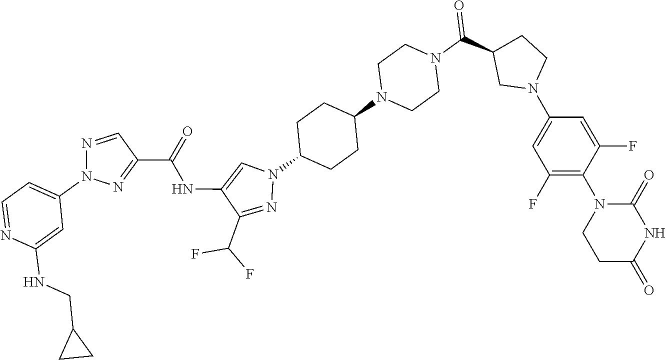 [SMILES: O=C1CCN(c2c(F)cc(N3CC[C@H](C(=O)N4CCN([C@H]5CC[C@H](n6cc(NC(=O)c7cnn(-c8ccnc(NCC9CC9)c8)n7)c(C(F)F)n6)CC5)CC4)C3)cc2F)C(=O)N1]

The title compound was prepared in a manner similar to that described in Example 51.

¹H NMR (500 MHz, DMSO) δ 10.47 (s, 1H), 10.22 (s, 1H), 8.60 (s, 1H), 8.16-8.14 (m, 2H), 7.25-7.03 (m, 4H), 6.31 (d, J=10.0 Hz, 2H), 4.26-4.20 (m, 1H), 3.60-3.44 (m, 10H), 3.37-3.35 (m, 2H), 3.28-3.26 (m, 1H), 3.20 (t, J=5.0 Hz, 2H), 2.68 (t, J=5.0 Hz, 2H), 2.60-2.54 (m, 3H), 2.18-2.06 (m, 4H), 1.98-1.90 (m, 2H), 1.85-1.77 (m, 2H), 1.48-1.41 (m, 2H), 1.07-1.06 (m, 1H), 0.46-0.45 (m, 2H), 0.23-0.22 (m, 2H). [M+H]⁺=863.2.

### Example 130: N-cyclopropyl-2-(trans)-4-(4-(1-(4-((R)-2,6-dioxopiperidin-3-yl)-3,5-difluorophenyl)azetidine-3-carbonyl)piperazin-1-yl)cyclohexyl)-5-(6-(trifluoromethyl)picolinamido)-2H-indazole-6-carboxamide

### Step 1: (cis)-4-(4-benzylpiperazin-1-yl)cyclohexan-1-ol

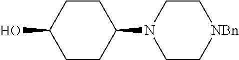 [SMILES: O[C@H]1CC[C@@H](N2CCN(Cc3ccccc3)CC2)CC1]

A mixture of N-benzyl-2-chloro-N-(2-chloroethyl)ethan-1-amine hydrochloride (8.04 g, 29.9 mmol) and (cis)-4-aminocyclohexan-1-ol (3.44 g, 29.9 mmol) in EtOH (180 mL) was added to Na₂CO₃ (11.09 g, 104.6 mmol). The mixture was stirred at 90° C. for 16 hours under nitrogen atmosphere. Then the solvent was removed by reduce pressure. The residue was washed by water and extracted with dichloromethane (3×150 mL). The organic phase was dried over anhydrous sodium sulfate, filtered and concentrated under reduced pressure to afford the crude product, which was purified with silica gel column chromatography (DCM:MeOH=100:0˜20:1 gradient elution) to yield the product (7.6 g, 93%). [M+H]⁺=275.6.

### Step 2: (cis)-4-(piperazin-1-yl)cyclohexan-1-ol

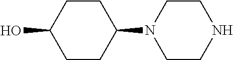 [SMILES: O[C@H]1CC[C@@H](N2CCNCC2)CC1]

A mixture of (cis)-4-(4-benzylpiperazin-1-yl)cyclohexan-1-ol (7.6 g, 27.7 mmol) in MeOH (100 mL) and AcOH (5 mL) was added to Pd/C (760 mg. 10% w). The reaction was stirred at 45° C. for 16 hours under hydrogen atmosphere. The mixture was then cooled. The mixture was filtered by celite and concentrated under reduced pressure to afford the product (4.9 g, 96%). [M+H]⁺=185.2.

### Step 3: tert-butyl 4-((cis)-4-hydroxycyclohexyl)piperazine-1-carboxylate

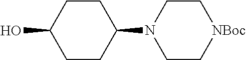 [SMILES: CC(C)(C)OC(=O)N1CCN([C@H]2CC[C@@H](O)CC2)CC1]

A mixture of (cis)-4-(piperazin-1-yl)cyclohexan-1-ol (4.9 g, 26.6 mmol) and (Boc)₂O (6.96 g, 31.9 mmol) in DCM (80 mL) was added to Et₃N (8.06 g, 79.8 mmol) at 0° C. The mixture was stirred at rt for 4 hour. The mixture was washed by water (150 mL) and extracted with dichloromethane (3×100 mL). The organic phase was dried over anhydrous sodium sulfate, filtered and concentrated under reduced pressure to afford the crude product, which was purified with silica gel column chromatography (DCM:MeOH=100:0˜20:1 gradient elution) to give the product (4.57 g, 60%). [M+H]⁺=285.6.

### Step 4: methyl 2-((trans)-4-(4-(tert-butoxycarbonyl)piperazin-1-yl)cyclohexyl)-5-nitro-2H-indazole-6-carboxylate

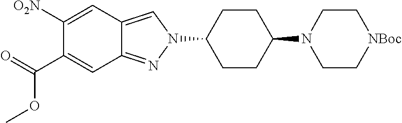 [SMILES: COC(=O)c1cc2nn([C@H]3CC[C@H](N4CCN(C(=O)OC(C)(C)C)CC4)CC3)cc2cc1[N+](=O)[O-]]

A mixture of tert-butyl 4-((cis)-4-hydroxycyclohexyl)piperazine-1-carboxylate (1.12 g, 3.93 mmol), methyl 5-nitro-2H-indazole-6-carboxylate (0.87 g, 3.93 mmol) (obtained through the same way described in WO2020035019 A1) and PPh₃ (1.55 g, 5.92 mmol) was dropwise to THF (50 mL) under nitrogen atmosphere at 0° C. The mixture was stirred at 0° C. for 15 min, and then DIAD (1.19 g, 5.92 mmol) was added. The reaction was stirred at 0° C. 1 hour. The mixture was allowed to slowly warm up to room temperature and stirred 16 h. The mixture was diluted with water (50 mL), extracted with dichloromethane (3×50 mL), and washed with brine (100 mL). The organic phase was dried over anhydrous sodium sulfate, filtered and concentrated under reduced pressure to afford the crude product, which was purified with silica gel column chromatography (DCM:MeOH=100:0˜20:1 gradient elution) to give the product (220 mg, 56%). [M+H]⁺=488.6. ¹H NMR (500 MHz, CDCl₃) δ 8.41 (s, 1H), 8.21 (s, 1H), 8.02 (s, 1H), 4.48-4.41 (m, 1H), 3.93 (s, 3H), 3.63-3.36 (m, 4H), 2.65-2.46 (m, 4H), 2.44-2.34 (m, 2H), 2.20-2.09 (m, 2H), 2.09-1.96 (m, 3H), 1.66-1.51 (m, 2H), 1.47 (s, 9H).

### Step 5: methyl 5-amino-2-((trans)-4-(4-(tert-butoxycarbonyl)piperazin-1-yl)cyclohexyl)-2H-indazole-6-carboxylate

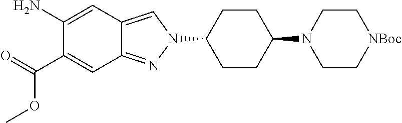 [SMILES: COC(=O)c1cc2nn([C@H]3CC[C@H](N4CCN(C(=O)OC(C)(C)C)CC4)CC3)cc2cc1N]

To a solution of methyl 2-((trans)-4-(4-(tert-butoxycarbonyl)piperazin-1-yl)cyclohexyl)-5-nitro-2H-indazole-6-carboxylate (220 mg, 0.45 mmol) in EtOH (10 mL) and Fe powder (126 mg, 2.26 mmol) was added NH₄Cl (121 mg. 2.26 mmol) (in water 3 mL). The reaction was stirred at 80° C. for 4 h. The mixture was then cooled. The mixture was filtered by celite and concentrated under reduced pressure to afford the crude product, which was purified with silica gel column chromatography (DCM:MeOH=100:0˜20:1 gradient elution) to afford the product (145 mg, 70%). [M+H]⁺=458.6.

### Step 6: methyl 2-(trans)-4-(4-(tert-butoxycarbonyl)piperazin-1-yl)cyclohexyl)-5-(6-(trifluoromethyl)picolinamido)-2H-indazole-6-carboxylate

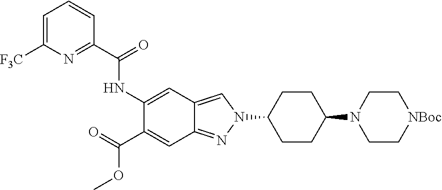 [SMILES: COC(=O)c1cc2nn([C@H]3CC[C@H](N4CCN(C(=O)OC(C)(C)C)CC4)CC3)cc2cc1NC(=O)c1cccc(C(F)(F)F)n1]

To a solution of 6-(trifluoromethyl)picolinic acid (61 mg, 0.32 mmol) in DMF (10 mL) was added HATU (133 mg, 0.35 mmol). The mixture was stirred at rt for 10 min. Then, methyl 5-amino-2-((trans)-4-(4-(tert-butoxycarbonyl)piperazin-1-yl)cyclohexyl)-2H-indazole-6-carboxylate (145 mg, 0.32 mmol) in Et₃N (96 mg, 0.95 mmol) was added to the reaction. The reaction was stirred at rt for 4 h. The solvent was removed under reduced pressure. The residue was washed water (50 mL), extracted with dichloromethane (3×50 mL), and washed with brine (100 mL). The organic phase was dried over anhydrous sodium sulfate, filtered and concentrated under reduced pressure to afford the crude product, which was purified with silica gel column chromatography (DCM:MeOH=100:0˜20:1 gradient elution) to afford the product (120 mg, 60%). [M+H]⁺=631.2

### Step 7: 2-(trans)-4-(4-(tert-butoxycarbonyl)piperazin-1-yl)cyclohexyl)-5-(6-(trifluoromethyl)picolinamido)-2H-indazole-6-carboxylicacid

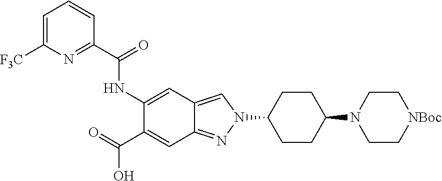 [SMILES: CC(C)(C)OC(=O)N1CCN([C@H]2CC[C@H](n3cc4cc(NC(=O)c5cccc(C(F)(F)F)n5)c(C(=O)O)cc4n3)CC2)CC1]

To a solution of methyl 2-(trans)-4-(4-(tert-butoxycarbonyl)piperazin-1-yl)cyclohexyl)-5-(6-(trifluoromethyl)picolinamido)-2H-indazole-6-carboxylate (120 mg, 0.19 mmol) in THF (2 mL)/MeOH (2 mL)/H₂O (2 mL) was added LiOH·H₂O (32 mg, 0.76 mmol). The mixture was stirred at rt for 5 h.

The solvent was removed by reduced pressure. The residue pH value was adjusted to 4-5 by 1N HCl. The mixture was filtered. The filter cake was concentrated under reduced pressure to afford the product (115 mg, 98%). [M+H]⁺=617.2

### Step 8: tert-butyl 4-((trans)-4-(6-(cyclopropylcarbamoyl)-5-(6-(trifluoromethyl)picolinamido)-2H-indazol-2-yl)cyclohexyl)piperazine-1-carboxylate

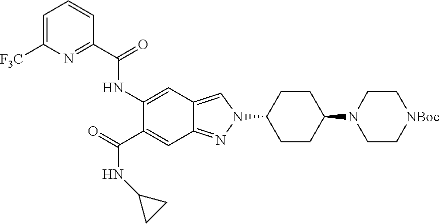 [SMILES: CC(C)(C)OC(=O)N1CCN([C@H]2CC[C@H](n3cc4cc(NC(=O)c5cccc(C(F)(F)F)n5)c(C(=O)NC5CC5)cc4n3)CC2)CC1]

To a solution of 2-(trans)-4-(4-(tert-butoxycarbonyl)piperazin-1-yl)cyclohexyl)-5-(6-(trifluoromethyl)picolinamido)-2H-indazole-6-carboxylic acid (90 mg, 0.15 mmol) in DMF (3 mL) was added HATU (61 mg, 0.16 mmol). The mixture was stirred at rt for 10 min. Then, cyclopropanamine (8 mg, 0.15 mmol) and Et₃N (46 mg, 0.45 mmol) was added to the reaction. The reaction was stirred at rt for 4 h. The solvent was removed by reduced pressure. The residue was washed water (15 mL), extracted with dichloromethane (3×20 mL), and washed with brine (30 mL). The organic phase was dried over anhydrous sodium sulfate, filtered and concentrated under reduced pressure to afford the crude product, which was purified with silica gel column chromatography (DCM:MeOH=100:0˜20:1 gradient elution) to give the product (70 mg, 73%). [M+H]⁺=656.6

### Step 9: N-cyclopropyl-2-(trans)-4-(piperazin-1-yl)cyclohexyl)-5-(6-(trifluoromethyl)picolinamido)-2H-indazole-6-carboxamide hydrochloride

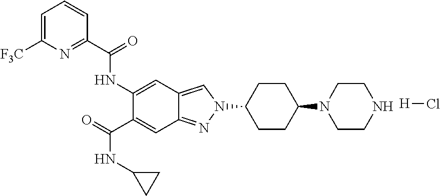

To a solution of tert-butyl 4-((trans)-4-(6-(cyclopropylcarbamoyl)-5-(6-(trifluoromethyl)picolinamido)-2H-indazol-2-yl)cyclohexyl)piperazine-1-carboxylate (70 mg, 0.11 mmol) in DCM (5 mL) was dropwise HCl/Dioxane (1 mL. 4N, 4 mmol). The reaction was stirred at rt for 4 h. The mixture was concentrated under reduced pressure to afford the product (55 mg, 92%). [M+H]⁺=556.3.

### Step 10: N-cyclopropyl-2-(trans)-4-(4-(1-(4-((R)-2,6-dioxopiperidin-3-yl)-3,5-difluorophenyl)azetidine-3-carbonyl)piperazin-1-yl)cyclohexyl)-5-(6-(trifluoromethyl)picolinamido)-2H-indazole-6-carboxamide

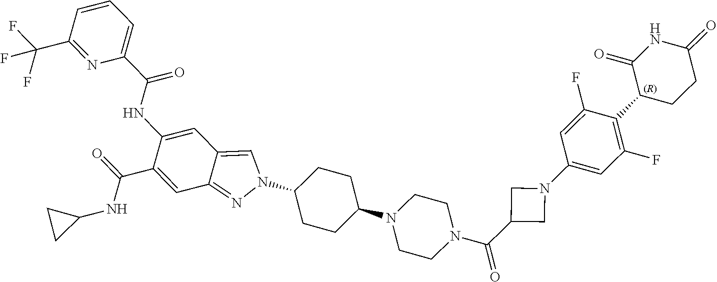

To a solution of (R)-1-(4-(2,6-dioxopiperidin-3-yl)-3,5-difluorophenyl)azetidine-3-carboxylic acid (14 mg, 0.042 mmol) in DMF (3 mL) was added HATU (18 mg, 0.046 mmol). The mixture was stirred at rt for 10 min. Then, N-cyclopropyl-2-(trans)-4-(piperazin-1-yl)cyclohexyl)-5-(6-(trifluoromethyl)picolinamido)-2H-indazole-6-carboxamide hydrochloride (25 mg, 0.042 mmol) and Et₃N (46 mg, 0.45 mmol) in DMF (2 mL) was added to the reaction. The reaction was stirred at rt for 4 h. The solvent was removed by reduced pressure. The residue was washed water (15 mL), extracted with dichloromethane (3×20 mL), and washed with brine (30 mL). The organic phase was dried over anhydrous sodium sulfate, filtered and concentrated under reduced pressure to afford the crude product, which was purified with silica gel column chromatography (DCM:MeOH=100:0˜10:1 gradient elution) to afford the product (5 mg, 14%). [M+H]⁺=862.6

¹H NMR (500 MHz, DMSO) δ 12.39 (s, 1H), 10.86 (s, 1H), 8.88 (s, 1H), 8.79 (d, J=4.3 Hz, 1H), 8.49 (s, 1H), 8.43 (d, J=7.9 Hz, 1H), 8.37 (t, J=7.8 Hz, 1H), 8.18 (d, J=7.7 Hz, 1H), 8.00 (s, 1H), 6.17 (d, J=11.1 Hz, 2H), 4.58-4.41 (m, 1H), 4.11-3.98 (m, 3H), 3.96-3.77 (m, 3H), 3.54-3.44 (m, 2H), 3.30-3.26 (m, 1H), 3.00-2.87 (m, 1H), 2.86-2.71 (m, 1H), 2.57-2.52 (m, 2H), 2.26-1.86 (m, 10H), 1.56-1.46 (m, 3H), 1.31-1.18 (m, 2H), 0.83 (dt, J=24.3, 8.6 Hz, 1H), 0.75-0.70 (m, 2H), 0.65-0.58 (m, 2H).

### Example 129: N-cyclopropyl-2-((1R,4r)-4-(4-((R)-1-(4-((R)-2,6-dioxopiperidin-3-yl)-3,5-difluorophenyl)pyrrolidine-3-carbonyl)piperazin-1-yl)cyclohexyl)-5-(6-(trifluoromethyl)picolinamido)-2H-indazole-6-carboxamide

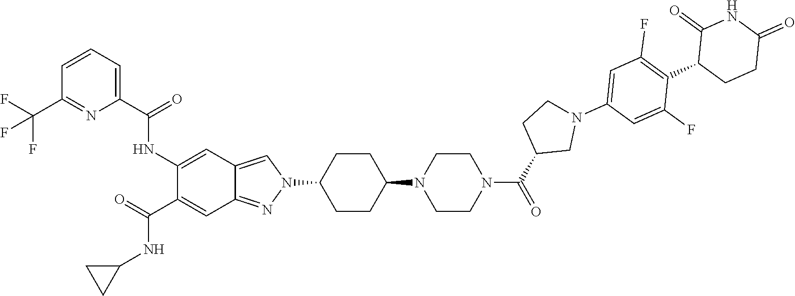 [SMILES: O=C1CC[C@H](c2c(F)cc(N3CC[C@@H](C(=O)N4CCN([C@H]5CC[C@H](n6cc7cc(NC(=O)c8cccc(C(F)(F)F)n8)c(C(=O)NC8CC8)cc7n6)CC5)CC4)C3)cc2F)C(=O)N1]

The title compound (5.24 mg, 20% yield) was prepared in a manner similar to that described in Example 130 from N-cyclopropyl-2-(trans)-4-(piperazin-1-yl)cyclohexyl)-5-(6-(trifluoromethyl)picolinamido)-2H-indazole-6-carboxamide hydrochloride. ¹H NMR (500 MHz, DMSO) δ 12.39 (s, 1H), 10.84 (s, 1H), 8.88 (s, 1H), 8.79 (d, J=4.2 Hz, 1H), 8.49 (s, 1H), 8.43 (d, J=7.4 Hz, 1H), 8.38 (d, J=7.9 Hz, 1H), 8.18 (d, J=7.7 Hz, 1H), 8.00 (s, 1H), 6.23 (d, J=12.2 Hz, 2H), 4.55-4.46 (m, 1H), 4.05-3.97 (m, 1H), 3.60-3.41 (m, 6H), 3.37-3.33 (m, 1H), 3.30-3.21 (m, 2H), 2.97-2.90 (m, 1H), 2.82-2.73 (m, 1H), 2.65-2.56 (m, 2H), 2.25-1.91 (m, 11H), 1.58-1.44 (m, 2H), 1.30-1.21 (m, 2H), 0.90-0.78 (m, 1H), 0.75-0.70 (m, 2H), 0.65-0.57 (m, 2H). [M+H]⁺=876.6

### Example 168: 2-((1R,4R)-4-(4-((R)-1-(4-((R)-2,6-dioxopiperidin-3-yl)-3,5-difluorophenyl)pyrrolidine-3-carbonyl)piperazin-1-yl)cyclohexyl)-N-((1r,3R)-3-(hydroxymethyl)cyclobutyl)-5-(6-(trifluoromethyl)picolinamido)-2H-indazole-6-carboxamide

### Step 1: tert-butyl 4-((1R,4r)-4-(6-(((1r,3R)-3-(hydroxymethyl)cyclobutyl)carbamoyl)-5-(6-(trifluoromethyl)picolinamido)-2H-indazol-2-yl)cyclohexyl)piperazine-1-carboxylate

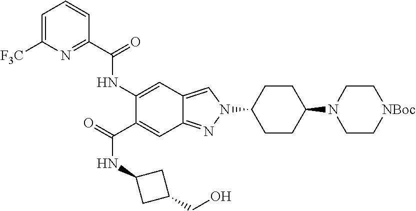 [SMILES: CC(C)(C)OC(=O)N1CCN([C@H]2CC[C@H](n3cc4cc(NC(=O)c5cccc(C(F)(F)F)n5)c(C(=O)N[C@H]5C[C@H](CO)C5)cc4n3)CC2)CC1]

To a solution of 2-(trans)-4-(4-(tert-butoxycarbonyl)piperazin-1-yl)cyclohexyl)-5-(6-(trifluoromethyl)picolinamido)-2H-indazole-6-carboxylic acid (62 mg, 0.10 mmol) in DMF (3 mL) was added HATU (42 mg, 0.11 mmol). The mixture was stirred at rt for 10 min. Then, ((1r,3r)-3-aminocyclobutyl)methanol 2,2,2-trifluoroacetate (22 mg, 0.10 mmol) and Et₃N (46 mg, 0.45 mmol) in DMF (3 mL) was added to the reaction. The reaction was stirred at rt for 4 h. The solvent was removed by reduced pressure. The residue was washed water (15 mL), extracted with dichloromethane (3×20 mL), and washed with brine (30 mL). The organic phase was dried over anhydrous sodium sulfate, filtered and concentrated under reduced pressure to afford the crude product, which was purified with silica gel column chromatography (DCM:MeOH=100:0˜10:1 gradient elution) to give the product (50 mg, 72%). [M+H]⁺=700.2

### Step 2: N-((1r,3R)-3-(hydroxymethyl)cyclobutyl)-2-((1r,4R)-4-(piperazin-1-yl)cyclohexyl)-5-(6-(trifluoromethyl)picolinamido)-2H-indazole-6-carboxamide hydrochloride

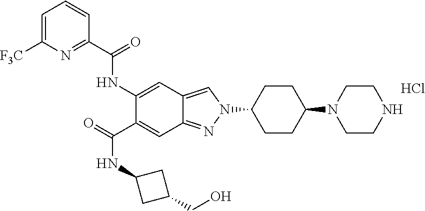 [SMILES: O=C(Nc1cc2cn([C@H]3CC[C@H](N4CCNCC4)CC3)nc2cc1C(=O)N[C@H]1C[C@H](CO)C1)c1cccc(C(F)(F)F)n1 ; Cl]

To a solution of tert-butyl 4-((1R,4r)-4-(6-(((1r,3R)-3-(hydroxymethyl)cyclobutyl)carbamoyl)-5-(6-(trifluoromethyl)picolinamido)-2H-indazol-2-yl)cyclohexyl)piperazine-1-carboxylate (50 mg, 0.07 mmol) in DCM (3 mL) was added dropwise HCl/Dioxane (1 mL. 4N, 4 mmol). The reaction was stirred at rt for 4 h. The mixture was then cooled. The mixture was concentrated under reduced pressure to afford the product (40 mg, 90%). [M+H]⁺=600.3.

### Step 3: 2-((1R,4R)-4-(4-((R)-1-(4-((R)-2,6-dioxopiperidin-3-yl)-3,5-difluorophenyl)pyrrolidine-3-carbonyl)piperazin-1-yl)cyclohexyl)-N-((1r,3R)-3-(hydroxymethyl)cyclobutyl)-5-(6-(trifluoromethyl)picolinamido)-2H-indazole-6-carboxamide

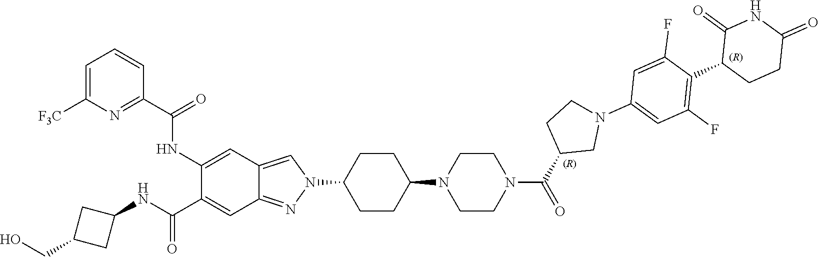

To a solution of (R)-1-(4-((R)-2,6-dioxopiperidin-3-yl)-3,5-difluorophenyl)pyrrolidine-3-carboxylic acid (9 mg, 0.017 mmol) in DMF (1 mL) was added HATU (11 mg, 0.03 mmol). The mixture was stirred at rt for 10 min. Then N-((1r,3R)-3-(hydroxymethyl)cyclobutyl)-2-((1r,4R)-4-(piperazin-1-yl)cyclohexyl)-5-(6-(trifluoromethyl)picolinamido)-2H-indazole-6-carboxamide hydrochloride (17 mg, 0.027 mmol) and Et₃N (46 mg, 0.45 mmol) in DMF (2 mL) was added to the reaction. The reaction was stirred at rt for 4 h. The solvent was removed by reduced pressure. The residue was diluted with water (15 mL), extracted with dichloromethane (3×20 mL), and washed with brine (30 mL). The organic phase was dried over anhydrous sodium sulfate, filtered and concentrated under reduced pressure to afford the crude product, which was purified with silica gel column chromatography (DCM:MeOH=100:0˜10:1 gradient elution) to afford the product (10 mg, 42%). [M+H]⁺=920.8

¹H NMR (500 MHz, DMSO) δ 12.40 (s, 1H), 10.84 (s, 1H), 9.02 (d, J=7.3 Hz, 1H), 8.89 (s, 1H), 8.50 (s, 1H), 8.45-8.34 (m, 2H), 8.17 (d, J=7.8 Hz, 1H), 8.08 (s, 1H), 6.23 (d, J=12.3 Hz, 2H), 4.60 (t, J=5.2 Hz, 1H), 4.55-4.41 (m, 2H), 4.06-4.00 (m, 1H), 3.60-3.42 (m, 8H), 3.37-3.33 (m, 1H), 3.30-3.24 (m, 2H), 3.15-2.95 (m, 3H), 2.84-2.73 (m, 2H), 2.24-2.06 (m, 9H), 2.01-1.91 (m, 5H), 1.86-1.78 (m, 2H), 1.68-1.61 (m, 1H), 1.58-1.47 (m, 2H).

### Example 167: 2-((1r,4R)-4-(4-(1-(4-((R)-2,6-dioxopiperidin-3-yl)-3,5-difluorophenyl)azetidine-3-carbonyl)piperazin-1-yl)cyclohexyl)-N-((1r,3R)-3-(hydroxymethyl)cyclobutyl)-5-(6-(trifluoromethyl)picolinamido)-2H-indazole-6-carboxamide

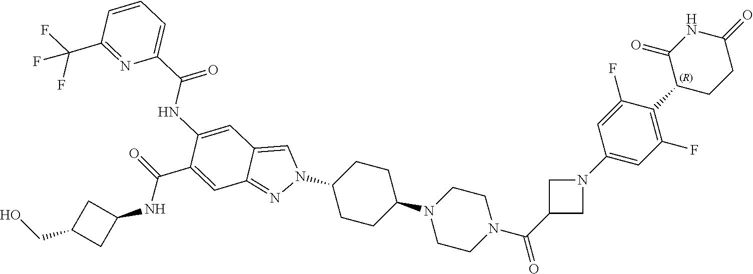

The title compound (7.24 mg, 30% yield) was prepared in a manner similar to that described in Example 168 from N-((1r,3R)-3-(hydroxymethyl)cyclobutyl)-2-((1r,4R)-4-(piperazin-1-yl)cyclohexyl)-5-(6-(trifluoromethyl)picolinamido)-2H-indazole-6-carboxamide hydrochloride. ¹H NMR (500 MHz, DMSO) δ 12.39 (s, 1H), 10.86 (s, 1H), 9.02 (d, J=7.2 Hz, 1H), 8.89 (s, 1H), 8.50 (s, 1H), 8.44-8.33 (m, 2H), 8.17 (d, J=7.8 Hz, 1H), 8.08 (s, 1H), 6.17 (d, J=11.1 Hz, 2H), 4.67-4.43 (m, 3H), 4.09-3.99 (m, 3H), 3.95-3.80 (m, 3H), 3.53-3.42 (m, 4H), 3.30-3.26 (m, 1H), 2.82-2.73 (m, 1H), 2.32-2.04 (m, 9H), 2.02-1.89 (m, 5H), 1.57-1.47 (m, 2H), 1.29-1.10 (m, 4H), 0.89-0.72 (m, 2H). [M+H]⁺=906.7

### Example 35: N-(2-(1-((1r,4r)-4-(4-(2,6-dioxopiperidin-3-yl)phenoxy)cyclohexane-1-carbonyl)piperidin-4-yl)-6-methoxy-2H-indazol-5-yl)-6-(trifluoromethyl)picolinamide

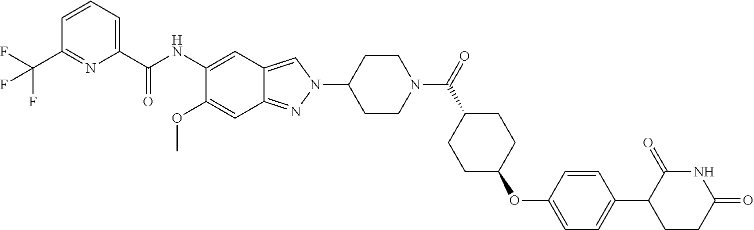 [SMILES: COc1cc2nn(C3CCN(C(=O)[C@H]4CC[C@H](Oc5ccc(C6CCC(=O)NC6=O)cc5)CC4)CC3)cc2cc1NC(=O)c1cccc(C(F)(F)F)n1]

The title compound was prepared in a manner similar to that described in Example 41.

¹H NMR (500 MHz, DMSO) δ 10.79 (s, 1H), 10.51 (s, 1H), 8.69 (s, 1H), 8.46 (d, J=7.8 Hz, 1H), 8.43-8.38 (m, 2H), 8.22 (d, J=7.7 Hz, 1H), 7.17 (s, 1H), 7.11 (d, J=8.6 Hz, 2H), 6.91 (d, J=8.6 Hz, 2H), 4.75-4.67 (m, 1H), 4.57-4.53 (m, 1H), 4.34-4.25 (m, 1H), 4.17-4.13 (m, 1H), 3.98 (s, 3H), 3.79-3.75 (m, 1H), 3.31-3.22 (m, 2H), 2.83-2.70 (m, 2H), 2.68-2.61 (m, 1H), 2.22-2.06 (m, 5H), 2.07-1.94 (m, 2H), 1.93-1.82 (m, 1H), 1.83-1.72 (m, 2H), 1.67-1.51 (m, 2H), 1.50-1.41 (m, 2H). [M+H]⁺=733.6.

### Example 36: N-(2-((1R,4r)-4-((4-((R)-1-(4-(2,4-dioxotetrahydropyrimidin-1(2H)-yl)phenyl)pyrrolidine-3-carbonyl)piperazin-1-yl)methyl)cyclohexyl)-6-methoxy-2H-indazol-5-yl)-6-(trifluoromethyl)picolinamide

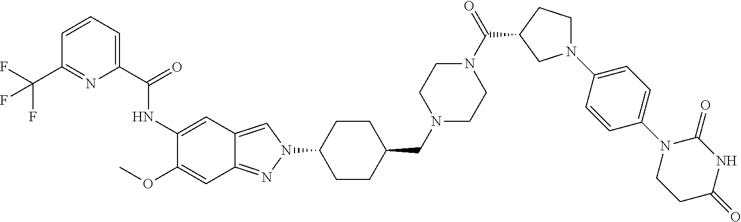 [SMILES: COc1cc2nn([C@H]3CC[C@H](CN4CCN(C(=O)[C@@H]5CCN(c6ccc(N7CCC(=O)NC7=O)cc6)C5)CC4)CC3)cc2cc1NC(=O)c1cccc(C(F)(F)F)n1]

The title compound was prepared in a manner similar to that described in Example 6.

¹H NMR (500 MHz, DMSO) δ 10.50 (s, 1H), 10.21 (s, 1H), 8.69 (s, 1H), 8.50-8.37 (m, 2H), 8.33 (s, 1H), 8.22 (d, J=7.7 Hz, 1H), 7.14 (dd, J=35.1, 10.4 Hz, 3H), 6.53 (d, J=8.7 Hz, 2H), 4.54-4.29 (m, 1H), 3.98 (s, 3H), 3.67 (t, J=6.6 Hz, 2H), 3.59-3.41 (m, 5H), 3.38-3.34 (m, 1H), 3.31-3.22 (m, 2H), 2.68 (t, J=6.6 Hz, 2H), 2.43-2.28 (m, 4H), 2.23-2.09 (m, 5H), 1.95-1.80 (m, 4H), 1.71-1.57 (m, 2H), 1.29-1.08 (m, 3H). [M+H]⁺=802.6

### Example 32: N-(2-((1r,4r)-4-(1-(2-(2,6-dioxopiperidin-3-yl)-1,3-dioxoisoindolin-5-yl)piperidine-4-carboxamido)cyclohexyl)-6-methoxy-2H-indazol-5-yl)-6-(trifluoromethyl)picolinamide

### Step 1: (1s,4s)-4-((tert-butoxycarbonyl)amino)cyclohexyl 4-methylbenzenesulfonate

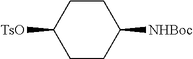 [SMILES: Cc1ccc(S(=O)(=O)O[C@H]2CC[C@@H](NC(=O)OC(C)(C)C)CC2)cc1]

To a mixture of tert-butyl ((1s,4s)-4-hydroxycyclohexyl)carbamate (5.0 g, 23.22 mmol) and DMAP (284 mg, 2.32 mmol) in pyridine (50.0 mL) was added TsCl (5.31 g, 27.87 mmol) at 0° C. and stirred at 20° C. under N₂ for 12 hours. The reaction mixture concentrated under reduced pressure. The residue was diluted with water (40.0 mL) and extracted with EtOAc (50.0 mL). The organic layer was washed with 0.1M HCl (50.0 mL), Na₂CO₃ (50.0 mL) and brine (20.0 mL), dried with anhydrous Na₂SO₄, filtered and concentrated in vacuum. The crude product was triturated with MTBE (50 ml) at 20° C. for 20 min and filtered to afford (1s,4s)-4-((tert-butoxycarbonyl)amino)cyclohexyl 4-methylbenzenesulfonate (4.2 g, 10.57 mmol, 45.52% yield). ¹HNMR (400 MHz, CDCl₃-d) δ ppm 7.79 (d, J=8.3 Hz, 2H), 7.34 (d, J=8.0 Hz, 2H), 4.66 (br s, 1H), 4.44 (br s, 1H), 3.46 (br s, 1H), 2.45 (s, 3H), 1.87 (br d, J=9.5 Hz, 2H), 1.80-1.72 (m, 2H), 1.62-1.55 (m, 2H), 1.54-1.49 (m, 2H), 1.43 (s, 9H); [M+Na]+=392.1.

### Step 2: tert-butyl ((1r,4r)-4-(6-methoxy-5-(6-(trifluoromethyl)picolinamido)-2H-indazol-2-yl)cyclohexyl)carbamate

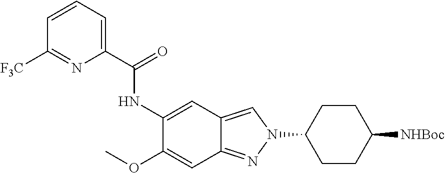 [SMILES: COc1cc2nn([C@H]3CC[C@H](NC(=O)OC(C)(C)C)CC3)cc2cc1NC(=O)c1cccc(C(F)(F)F)n1]

To a solution of (1s,4s)-4-((tert-butoxycarbonyl)amino)cyclohexyl 4-methylbenzenesulfonate (4.0 g, 10.83 mmol) and N-(6-methoxy-2H-indazol-5-yl)-6-(trifluoromethyl)pyridine-2-carboxamide (728 mg, 2.17 mmol) in DMF (50.0 mL) was added Cs₂CO₃ (3.5 g, 10.83 mmol). The mixture was stirred at 80° C. for 12 hours. The reaction mixture was quenched with H₂O (5.0 mL) at 20° C. and then extracted with EtOAc (5.0 mL*3). The combined organic layer was washed with brine (5 mL*3), dried over Na₂SO₄, filtered and concentrated under reduced pressure to afford residue, which was purified with prep-HPLC ([water (NH₄HCO₃)-ACN]; B %: 55%-75%, 20 min) to afford tert-butyl ((1r,4r)-4-(6-methoxy-5-(6-(trifluoromethyl)picolinamido)-2H-indazol-2-yl)cyclohexyl)carbamate (300.0 mg, 0.545 mmol, 25% yield). ¹HNMR (400 MHz, CDCl₃-d) δ ppm 10.74-10.67 (m, 1H), 8.82 (s, 1H), 8.50 (d, J=7.8 Hz, 1H), 8.12 (t, J=7.9 Hz, 1H), 7.86 (t, J=3.8 Hz, 2H), 7.07 (s, 1H), 4.46 (br d, J=3.5 Hz, 1H), 4.37-4.27 (m, 1H), 4.03 (s, 3H), 3.68-3.54 (m, 1H), 2.37-2.23 (m, 4H), 2.13-2.03 (m, 2H), 1.47 (s, 9H), 1.38 (br dd, J=2.9, 12.1 Hz, 2H); [M+H]⁺=534.4.

### Step 3: N-(2-((1r,4r)-4-aminocyclohexyl)-6-methoxy-2H-indazol-5-yl)-6-(trifluoromethyl)picolinamide, Hydrochloride

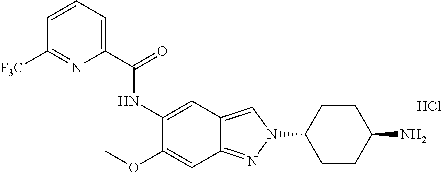 [SMILES: COc1cc2nn([C@H]3CC[C@H](N)CC3)cc2cc1NC(=O)c1cccc(C(F)(F)F)n1 ; Cl]

To a solution of tert-butyl ((1r,4r)-4-(6-methoxy-5-(6-(trifluoromethyl)picolinamido)-2H-indazol-2-yl)cyclohexyl)carbamate (280.0 mg, 0.525 mmol) in DCM (4.0 mL) was added HCl/EtOAc (4 M, 1.3 mL). The mixture was stirred at 20° C. for 1 hour. The reaction mixture was filtered and concentrated under reduced pressure to give the product. N-(2-((1r,4r)-4-aminocyclohexyl)-6-methoxy-2H-indazol-5-yl)-6-(trifluoromethyl)picolinamide, Hydrochloride (235.0 mg, 0.475 mmol, 91% yield) was obtained. ¹HNMR (400 MHz, DMSO-d6) δ pmm 10.50 (s, 1H), 8.69 (s, 1H), 8.48-8.44 (m, 1H), 8.43-8.38 (m, 1H), 8.34 (s, 1H), 8.22 (dd, J=1.1, 7.6 Hz, 1H), 8.09-8.05 (m, 2H), 7.14 (s, 1H), 4.43 (s, 1H), 4.01-3.94 (m, 3H), 3.18-3.11 (m, 1H), 2.22-2.07 (m, 4H), 2.05-1.93 (m, 2H), 1.64-1.49 (m, 2H); [M+H]⁺=434.2.

### Step 4: N-(2-((1r,4r)-4-(1-(2-(2,6-dioxopiperidin-3-yl)-1,3-dioxoisoindolin-5-yl)piperidine-4-carboxamido)cyclohexyl)-6-methoxy-2H-indazol-5-yl)-6-(trifluoromethyl)picolinamide

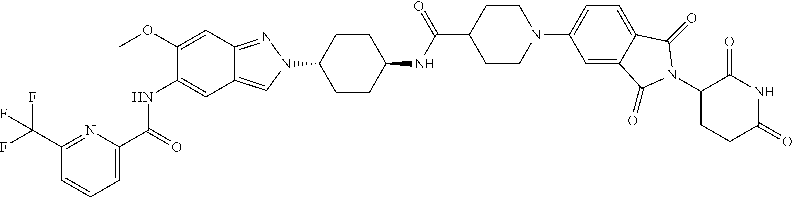 [SMILES: COc1cc2nn([C@H]3CC[C@H](NC(=O)C4CCN(c5ccc6c(c5)C(=O)N(C5CCC(=O)NC5=O)C6=O)CC4)CC3)cc2cc1NC(=O)c1cccc(C(F)(F)F)n1]

To a solution of 1-(2-(2,6-dioxopiperidin-3-yl)-1,3-dioxoisoindolin-5-yl)piperidine-4-carboxylic acid (38.5 mg, 0.10 mmol) in DMF (3 mL) was added HATU (42 mg, 0.11 mmol). The mixture was stirred at rt for 10 min. Then, N-(2-((1r,4r)-4-aminocyclohexyl)-6-methoxy-2H-indazol-5-yl)-6-(trifluoromethyl)picolinamide, Hydrochloride (47 mg, 0.10 mmol) and Et₃N (46 mg, 0.45 mmol) in DMF (3 mL) was added to the reaction. The reaction was stirred at rt for 4 h. The solvent was removed by reduced pressure. The residue was diluted with water (15 mL), extracted with dichloromethane (3×20 mL), and washed with brine (30 mL). The organic phase was dried over anhydrous sodium sulfate, filtered and concentrated under reduced pressure to afford the crude product, which was purified with silica gel column chromatography (DCM:MeOH=100:0˜10:1 gradient elution) to yield the product (12 mg, 14%). [M+H]⁺=801.5; ¹H NMR (500 MHz, DMSO) δ 11.08 (s, 1H), 10.50 (s, 1H), 8.68 (s, 1H), 8.49-8.38 (m, 2H), 8.35 (s, 1H), 8.22 (d, J=7.5 Hz, 1H), 7.82 (d, J=5 Hz, 1H), 7.67 (d, J=5 Hz, 1H), 7.34 (s, 1H), 7.26 (d, J=10 Hz, 1H), 7.17 (s, 1H), 5.09-5.03 (m, 1H), 4.50-4.35 (m, 1H), 4.17-4.03 (m, 2H), 3.98 (s, 3H), 3.71-3.60 (m, 1H), 3.06-2.96 (m, 2H), 2.89-2.84 (m, 1H), 2.65-2.54 (m, 2H), 2.43-2.38 (m, 1H), 2.17-2.09 (m, 2H), 2.05-1.91 (m, 5H), 1.82-1.73 (m, 2H), 1.68-1.57 (m, 2H), 1.50-1.36 (m, 2H).

### Example 41: (R)—N-(2-(1-(1-(1-(4-(2,6-dioxopiperidin-3-yl)-3,5-difluorophenyl)azetidine-3-carbonyl)azetidin-3-yl)piperidin-4-yl)-6-methoxy-2H-indazol-5-yl)-6-(trifluoromethyl)picolinamide

### Step 1: N-(6-methoxy-2H-indazol-5-yl)-6-(trifluoromethyl)picolinamide

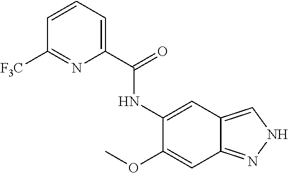 [SMILES: COc1cc2n[nH]cc2cc1NC(=O)c1cccc(C(F)(F)F)n1]

A mixture of 6-methoxy-2H-indazol-5-amine (2 g, 12.27 mmol), 6-(trifluoromethyl)picolinic acid (2.34 g, 12.27 mmol), T₃P (12 mL) and DIEA (2.37 g, 18.4 mmol) in THF (60 mL) was stirred at room temperature for 16 h. Reaction was monitored with LCMS. The resulting solution was extracted with DCM (2×50.0 mL) and washed with water (60 mL). The combined organic layer was washed with brine (50 mL), dried over anhydrous Na₂SO₄, filtered, and concentrated under reduced pressure to afford the crude product, which was further purified with silica gel column chromatography (PE:EA=5:1˜1:2 gradient elution) to yield the product (2 g, 48.5%). [M+H]⁺=337.1.

### Step 2: tert-butyl 4-(6-methoxy-5-(6-(trifluoromethyl)picolinamido)-2H-indazol-2-yl)piperidine-1-carboxylate

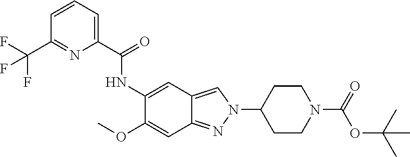 [SMILES: COc1cc2nn(C3CCN(C(=O)OC(C)(C)C)CC3)cc2cc1NC(=O)c1cccc(C(F)(F)F)n1]

A mixture of N-(6-methoxy-2H-indazol-5-yl)-6-(trifluoromethyl)picolinamide (2 g, 6 mmol), tert-butyl 4-hydroxypiperidine-1-carboxylate (3.6 g, 18 mmol) and Cyanomethylenetributylphosphorane (4.3 g, 18 mmol) in PhMe (80 mL) was stirred in a round bottom flask at 100° C. overnight. The mixture was evaporated under vacuum to afford the crude product, which was further purified with silica gel column chromatography (PE:EA=3: 1-1:5 gradient elution) to yield the product (1.7 g, 54.6%). [M+H]⁺=520.5.

### Step 3: N-(6-methoxy-2-(piperidin-4-yl)-2H-indazol-5-yl)-6-(trifluoromethyl)picolinamide hydrochloride

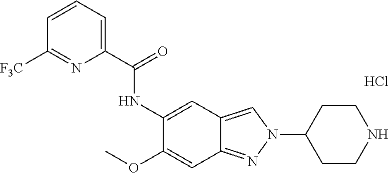 [SMILES: COc1cc2nn(C3CCNCC3)cc2cc1NC(=O)c1cccc(C(F)(F)F)n1 ; Cl]

Tert-butyl 4-(6-methoxy-5-(6-(trifluoromethyl)picolinamido)-2H-indazol-2-yl)piperidine-1-carboxylate (1.7 g, 3.27 mmol) in DCM (15 mL) was added 4N HCl/dioxane (30 mL) at 25° C. The mixture was stirred at 25° C. for 1 h. Reaction was monitored with LCMS. The solid was filtrated, washed by DCM (20.0 mL), and dried to obtain the product (1.3 g, 94.9%). [M+H]⁺=420.2.

### Step 4: tert-butyl 3-(4-(6-methoxy-5-(6-(trifluoromethyl)picolinamido)-2H-indazol-2-yl)piperidin-1-yl)azetidine-1-carboxylate

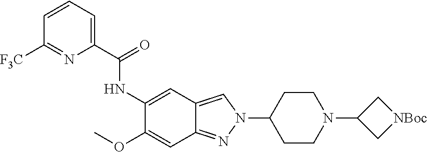 [SMILES: COc1cc2nn(C3CCN(C4CN(C(=O)OC(C)(C)C)C4)CC3)cc2cc1NC(=O)c1cccc(C(F)(F)F)n1]

A mixture of N-(6-methoxy-2-(piperidin-4-yl)-2H-indazol-5-yl)-6-(trifluoromethyl)picolinamide hydrochloride (300 mg, 0.66 mmol) and tert-butyl 3-oxoazetidine-1-carboxylate (169 mg, 0.99 mmol) in DCM (30 mL) was stirred in a round bottom flask for 16 h at room temperature. Then NaBH(AcO)₃ (419 mg, 1.98 mmol) was added and stirred for 3 h at room temperature. The reaction was quenched with water and the mixture was washed once with saturated aqueous NaHCO₃, then extracted with DCM. The organic layer was dried over anhydrous Na₂SO₄, and evaporated under vacuum to afford the product (300 mg, 79.1%). [M+H]⁺=575.3.

### Step 5: N-(2-(1-(azetidin-3-yl)piperidin-4-yl)-6-methoxy-2H-indazol-5-yl)-6-(trifluoromethyl)picolinamide trifluoroacetate

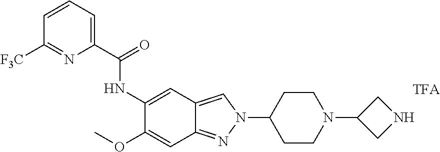 [SMILES: COc1cc2nn(C3CCN(C4CNC4)CC3)cc2cc1NC(=O)c1cccc(C(F)(F)F)n1]

Tert-butyl 3-(4-(6-methoxy-5-(6-(trifluoromethyl)picolinamido)-2H-indazol-2-yl)piperidin-1-yl)azetidine-1-carboxylate (300 mg, 0.52 mmol) in DCM (8 mL) was added TFA (20 mL) at 25° C. The mixture was stirred at 25° C. for 2 h. Reaction was monitored with LCMS. The solid was filtrated, washed with DCM (20.0 mL), and dried to obtain the product (300 mg, 98%). [M+H]⁺=475.3.

### Step 6: (R)—N-(2-(1-(1-(1-(4-(2,6-dioxopiperidin-3-yl)-3,5-difluorophenyl)azetidine-3-carbonyl)azetidin-3-yl)piperidin-4-yl)-6-methoxy-2H-indazol-5-yl)-6-(trifluoromethyl)picolinamide

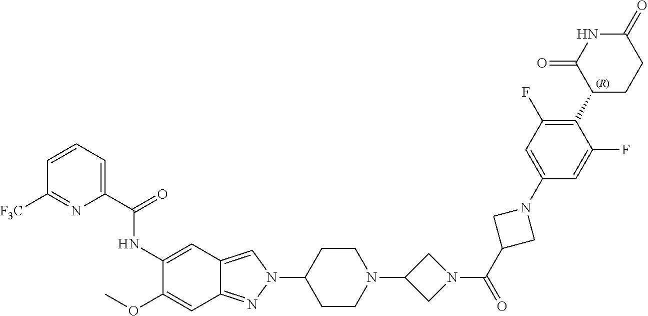

A mixture of (R)-1-(4-(2,6-dioxopiperidin-3-yl)-3,5-difluorophenyl)azetidine-3-carboxylic acid (30 mg, 0.093 mmol), HATU (43 mg, 0.112 mmol) and DIEA (36 mg, 0.279 mmol) in DMF (8 mL) was stirred at room temperature for 15 minute, and N-(2-(1-(azetidin-3-yl)piperidin-4-yl)-6-methoxy-2H-indazol-5-yl)-6-(trifluoromethyl)picolinamide trifluoroacetate (55 mg, 0.093 mmol) was added. The mixture was stirred at room temperature for 16 h. Reaction was monitored by LCMS. The resulting solution was extracted with DCM (2×15.0 mL) and water (20 mL). The organic layers were dried over anhydrous sodium sulfate and concentrated under vacuum to give the crude product. The crude product was purified with Prep-HPLC to afford the product (6 mg, 8%). ¹H NMR (500 MHz, DMSO) δ 10.86 (s, 1H), 10.52 (s, 1H), 8.71 (s, 1H), 8.47 (d, J=7.7 Hz, 1H), 8.44-8.37 (m, 2H), 8.23 (d, J=7.7 Hz, 1H), 7.17 (s, 1H), 6.17 (d, J=11.2 Hz, 2H), 4.53-4.29 (m, 1H), 4.26-4.09 (m, 2H), 4.10-3.92 (m, 7H), 3.90-3.80 (m, 2H), 3.66-3.54 (m, 2H), 3.21-3.10 (m, 2H), 3.02-2.85 (m, 1H), 2.83-2.73 (m, 1H), 2.48-2.22 (m, 4H), 2.20-2.01 (m, 4H), 1.99-1.89 (m, 1H). [M+H]⁺=781.5.

### Example 42: N-(2-(1-(1-((R)-1-(4-((R)-2,6-dioxopiperidin-3-yl)-3,5-difluorophenyl)pyrrolidine-3-carbonyl)azetidin-3-yl)piperidin-4-yl)-6-methoxy-2H-indazol-5-yl)-6-(trifluoromethyl)picolinamide

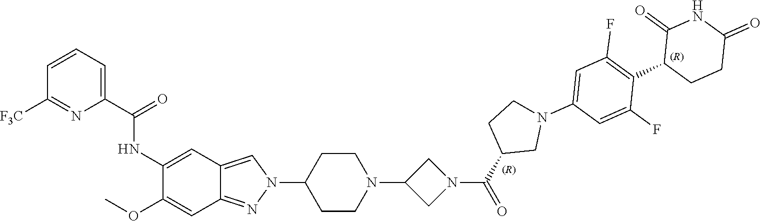

The title compound was prepared in a manner similar to that described in Example 41. ¹H NMR (500 MHz, DMSO) δ 10.77 (s, 1H), 10.44 (s, 1H), 8.63 (s, 1H), 8.40 (d, J=7.8 Hz, 1H), 8.37-8.30 (m, 2H), 8.15 (d, J=7.7 Hz, 1H), 7.10 (s, 1H), 6.17 (dd, J=12.0, 8.5 Hz, 2H), 4.43-4.33 (m, 1H), 4.27-4.17 (m, 1H), 4.09-4.00 (m, 1H), 3.99-3.90 (m, 4H), 3.86 (dt, J=14.9, 4.2 Hz, 1H), 3.73-3.61 (m, 1H), 3.44-3.34 (m, 1H), 3.23-3.06 (m, 6H), 2.95-2.82 (m, 2H), 2.77-2.65 (m, 1H), 2.16-1.94 (m, 9H), 1.93-1.82 (m, 1H). [M+H]⁺=795.6.

### Example 43: N-(2-(1′-((R)-1-(4-((R)-2,6-dioxopiperidin-3-yl)-3,5-difluorophenyl)pyrrolidine-3-carbonyl)-[1,4′-bipiperidin]-4-yl)-6-methoxy-2H-indazol-5-yl)-6-(trifluoromethyl)picolinamide

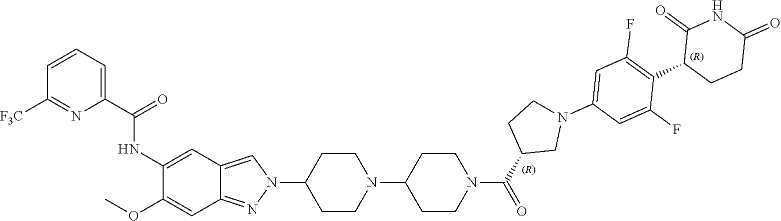

The title compound was prepared in a manner similar to that described in Example 41. ¹H NMR (500 MHz, DMSO) δ 10.84 (s, 1H), 10.52 (s, 1H), 8.72 (s, 1H), 8.47 (d, J=7.7 Hz, 1H), 8.40 (dd, J=15.8, 7.9 Hz, 2H), 8.23 (d, J=7.7 Hz, 1H), 7.16 (s, 1H), 6.23 (d, J=12.8 Hz, 2H), 4.87-4.69 (m, 1H), 4.65-4.47 (m, 1H), 4.30-4.12 (m, 1H), 4.08-3.93 (m, 4H), 3.75-3.51 (m, 3H), 3.49-3.43 (m, 2H), 3.42-3.34 (m, 2H), 3.17-3.05 (m, 2H), 2.85-2.72 (m, 1H), 2.68-2.52 (m, 2H), 2.47-2.29 (m, 5H), 2.22-2.01 (m, 6H), 1.99-1.89 (m, 1H), 1.76-1.40 (m, 2H). [M+H]⁺=823.7.

### Example 44: N-(2-(1′-(1-(4-(2,4-dioxotetrahydropyrimidin-1(2H)-yl)phenyl)azetidine-3-carbonyl)-[1,4′-bipiperidin]-4-yl)-6-methoxy-2H-indazol-5-yl)-6-(trifluoromethyl)picolinamide

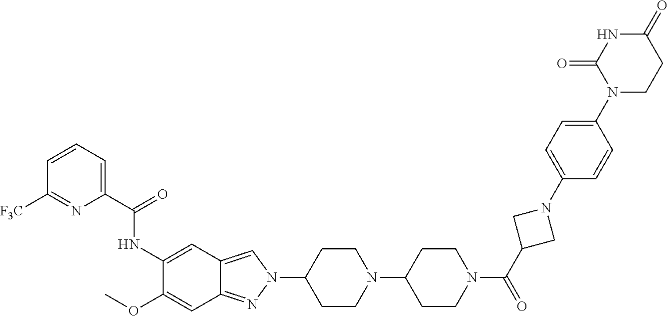 [SMILES: COc1cc2nn(C3CCN(C4CCN(C(=O)C5CN(c6ccc(N7CCC(=O)NC7=O)cc6)C5)CC4)CC3)cc2cc1NC(=O)c1cccc(C(F)(F)F)n1]

The title compound was prepared in a manner similar to that described in Example 41. ¹H NMR (500 MHz, DMSO) δ 10.50 (s, 1H), 10.23 (s, 1H), 8.69 (s, 1H), 8.46 (d, J=7.6 Hz, 1H), 8.41 (t, J=7.8 Hz, 1H), 8.37 (s, 1H), 8.22 (d, J=7.6 Hz, 1H), 7.16 (s, 1H), 7.11 (d, J=8.5 Hz, 2H), 6.46 (d, J=8.6 Hz, 2H), 4.48-4.29 (m, 2H), 4.10-3.95 (m, 5H), 3.93-3.78 (m, 3H), 3.74-3.58 (m, 3H), 3.07-2.94 (m, 3H), 2.76-2.64 (m, 2H), 2.65-2.59 (m, 2H), 2.40-2.30 (m, 2H), 2.16-1.98 (m, 4H), 1.85-1.74 (m, 2H), 1.47-1.37 (m, 1H), 1.36-1.28 (m, 1H). [M+H]⁺=774.8.

### Example 45: (R)—N-(2-(1′-(1-(4-(2,6-dioxopiperidin-3-yl)-3,5-difluorophenyl)azetidine-3-carbonyl)-[1,4′-bipiperidin]-4-yl)-6-methoxy-2H-indazol-5-yl)-6-(trifluoromethyl)picolinamide

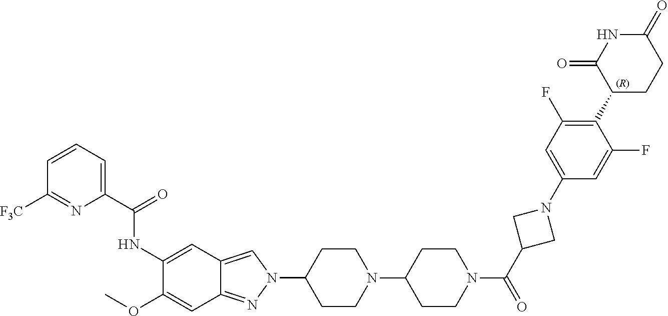

The title compound was prepared in a manner similar to that described in Example 41. ¹H NMR (500 MHz, DMSO) δ 10.79 (s, 1H), 10.44 (s, 1H), 8.62 (s, 1H), 8.40 (d, J=7.7 Hz, 1H), 8.34 (t, J=7.8 Hz, 1H), 8.30 (s, 1H), 8.15 (d, J=7.7 Hz, 1H), 7.09 (s, 1H), 6.10 (d, J=11.1 Hz, 2H), 4.45-4.28 (m, 2H), 4.02-3.93 (m, 3H), 3.92 (s, 3H), 3.89-3.82 (m, 3H), 3.81-3.73 (m, 1H), 3.66-3.56 (m, 1H), 3.06-2.88 (m, 4H), 2.76-2.66 (m, 1H), 2.60-2.50 (m, 3H), 2.14-1.93 (m, 5H), 1.93-1.82 (m, 1H), 1.81-1.68 (m, 2H), 1.44-1.21 (m, 2H). [M+H]⁺=809.5.

### Example 102: 1-cyclopropyl-N-(2-((1r,4r)-4-(4-(1-(4-((R)-2,6-dioxopiperidin-3-yl)-3,5-difluorophenyl)azetidine-3-carbonyl)piperazin-1-yl)cyclohexyl)-6-(2-hydroxypropan-2-yl)-2H-indazol-5-yl)-2-oxo-1,2-dihydropyridine-3-carboxamide

### Step 1: methyl 2-((1r,4r)-4-(4-(tert-butoxycarbonyl)piperazin-1-yl)cyclohexyl)-2H-indazole-6-carboxylate

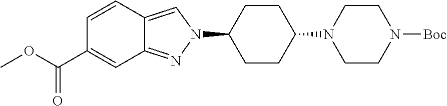 [SMILES: COC(=O)c1ccc2cn([C@H]3CC[C@H](N4CCN(C(=O)OC(C)(C)C)CC4)CC3)nc2c1]

To a solution of methyl 4-formyl-3-nitrobenzoate (4.2 g, 20 mmol) in propan-2-ol (100 mL) was added tert-butyl 4-((1r,4r)-4-aminocyclohexyl)piperazine-1-carboxylate (5.66 g, 20 mmol). The mixture was stirred for 3 hrs at 80° C. under N₂. To the above solution was added tributylphosphane (12.12 g, 60 mmol). The mixture was stirred overnight at 80° C. under N₂. The mixture was cooled to rt and concentrated under vacuum. The residue was purified by silica gel column chromatography (DCM:MeOH=30:1) to yield product (7 g, 79%). [M+H]⁺=443.3

### Step 2: methyl 5-nitro-2-((1r,4r)-4-(piperazin-1-yl)cyclohexyl)-2H-indazole-6-carboxylate

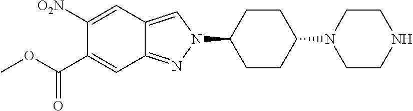 [SMILES: COC(=O)c1cc2nn([C@H]3CC[C@H](N4CCNCC4)CC3)cc2cc1[N+](=O)[O-]]

To a solution of methyl 2-((1r,4r)-4-(4-(tert-butoxycarbonyl)piperazin-1-yl)cyclohexyl)-2H-indazole-6-carboxylate (7 g, 15.84 mmol) in conc.H₂SO₄ (80 mL) was slowed added NaNO₃ (1.615 g, 19 mmol) at 0° C. The mixture was stirred for 2 hrs at 0° C. The mixture was quenched with ice-water, then adjusted to pH 9 with sat.Na₂CO₃ solution and extracted with DCM. The combined organic layer was dried over anhydrous Na₂SO₄, filtered and evaporated in vacuum to afford product (4.8 g, 78%), which was used without further purification. [M+H]⁺=388.2

### Step 3: methyl 2-((1r,4r)-4-(4-(tert-butoxycarbonyl)piperazin-1-yl)cyclohexyl)-5-nitro-2H-indazole-6-carboxylate

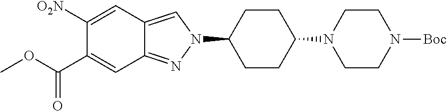 [SMILES: COC(=O)c1cc2nn([C@H]3CC[C@H](N4CCN(C(=O)OC(C)(C)C)CC4)CC3)cc2cc1[N+](=O)[O-]]

To a solution of methyl 5-nitro-2-((1r,4r)-4-(piperazin-1-yl)cyclohexyl)-2H-indazole-6-carboxylate (4.8 g, 12.4 mmol) in DCM (60 mL) was added Et₃N (3.76 g, 37.2 mmol) and Boc₂O (4.06 g, 18.6 mmol). The reaction mixture was stirred overnight at rt. The mixture was concentrated under vacuum. The residue was purified with silica gel column chromatography (DCM:MeOH=30:1) to yield product (4.9 g, 81%). [M+H]⁺=488.2

### Step 4: methyl 5-amino-2-((1r,4r)-4-(4-(tert-butoxycarbonyl)piperazin-1-yl)cyclohexyl)-2H-indazole-6-carboxylate

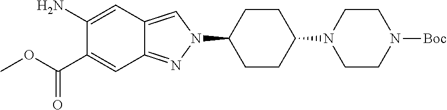 [SMILES: COC(=O)c1cc2nn([C@H]3CC[C@H](N4CCN(C(=O)OC(C)(C)C)CC4)CC3)cc2cc1N]

To a solution of methyl 2-((1r,4r)-4-(4-(tert-butoxycarbonyl)piperazin-1-yl)cyclohexyl)-5-nitro-2H-indazole-6-carboxylate (4.9 g, 10.06 mmol) in MeOH (50 mL) and water (10 mL) was added NH₄Cl (2.69 g, 50.3 mmol) and Fe powder (2.82 g, 50.3 mmol) at room temperature. The resulting mixture was stirred for 2 hrs at 70° C. The mixture was cooled to rt and concentrated under vacuum. The residue was purified by silica gel column chromatography (DCM:MeOH=15:1) to afford product (3.2 g, 70%). [M+H]⁺=458.3

### Step 5: tert-butyl 4-((1r,4r)-4-(5-amino-6-(2-hydroxypropan-2-yl)-2H-indazol-2-yl)cyclohexyl)piperazine-1-carboxylate

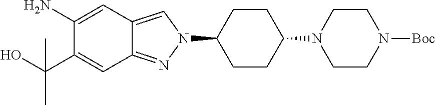 [SMILES: CC(C)(C)OC(=O)N1CCN([C@H]2CC[C@H](n3cc4cc(N)c(C(C)(C)O)cc4n3)CC2)CC1]

To a solution of methyl 5-amino-2-((1r,4r)-4-(4-(tert-butoxycarbonyl)piperazin-1-yl)cyclohexyl)-2H-indazole-6-carboxylate (1.2 g, 2.62 mmol) and LiCl (0.557 g, 13.1 mmol) in THF (20 mL) was added dropwise MeMgBr in THF (1 M, 26.2 mL, 26.2 mmol) at 0° C. The resulting mixture was stirred for 2 hrs at 0° C. The mixture was quenched with sat.NH₄Cl solution and extracted with DCM. The combined organic layer was dried over anhydrous Na₂SO₄, filtered and evaporated under vacuum. The residue was purified on silica gel column chromatography (DCM:MeOH=10:1) to afford product (0.48 g, 40%). [M+H]⁺=458.3

### Step 6: tert-butyl 4-((1r,4r)-4-(5-(1-cyclopropyl-2-oxo-1,2-dihydropyridine-3-carboxamido)-6-(2-hydroxypropan-2-yl)-2H-indazol-2-yl)cyclohexyl)piperazine-1-carboxylate

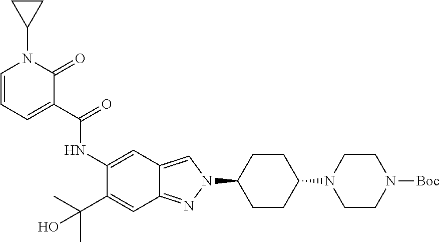 [SMILES: CC(C)(C)OC(=O)N1CCN([C@H]2CC[C@H](n3cc4cc(NC(=O)c5cccn(C6CC6)c5=O)c(C(C)(C)O)cc4n3)CC2)CC1]

To a solution of tert-butyl 4-((1r,4r)-4-(5-amino-6-(2-hydroxypropan-2-yl)-2H-indazol-2-yl)cyclohexyl)piperazine-1-carboxylate (0.48 g, 1.05 mmol) and 1-cyclopropyl-2-oxo-1,2-dihydropyridine-3-carboxylic acid (0.189 g, 1.05 mmol) in DMF (10 mL) was added DIPEA (0.406 g, 3.15 mmol) and HATU (0.4 g, 1.05 mmol) at rt. The resulting mixture was stirred for 2 hrs at rt. The mixture was diluted with water and extracted with DCM. The combined organic layer was dried over anhydrous Na₂SO₄, filtered and evaporated under vacuum. The residue was purified with silica gel column chromatography (DCM:MeOH=20:1) to afford product (0.4 g, 62%). [M+H]⁺=619.4

### Step 7: 1-cyclopropyl-N-(6-(2-hydroxypropan-2-yl)-2-((1r,4r)-4-(piperazin-1-yl)cyclohexyl)-2H-indazol-5-yl)-2-oxo-1,2-dihydropyridine-3-carboxamide

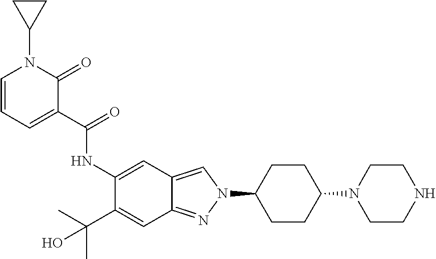 [SMILES: CC(C)(O)c1cc2nn([C@H]3CC[C@H](N4CCNCC4)CC3)cc2cc1NC(=O)c1cccn(C2CC2)c1=O]

To a solution of tert-butyl 4-((1r,4r)-4-(5-(1-cyclopropyl-2-oxo-1,2-dihydropyridine-3-carboxamido)-6-(2-hydroxypropan-2-yl)-2H-indazol-2-yl)cyclohexyl)piperazine-1-carboxylate (0.4 g, 0.647 mmol) in DCM (10 mL) was added TFA (2 mL) at rt. The resulting mixture was stirred for 1 h at rt. The mixture was quenched with sat.NaHCO₃ solution and extracted with DCM. The combined organic layer was dried over anhydrous Na₂SO₄, filtered and evaporated under vacuum to afford product (0.275 g, 82%). [M+H]⁺=519.3

### Step 8: 1-cyclopropyl-N-(2-((1r,4r)-4-(4-(1-(4-((R)-2,6-dioxopiperidin-3-yl)-3,5-difluorophenyl)azetidine-3-carbonyl)piperazin-1-yl)cyclohexyl)-6-(2-hydroxypropan-2-yl)-2H-indazol-5-yl)-2-oxo-1,2-dihydropyridine-3-carboxamide

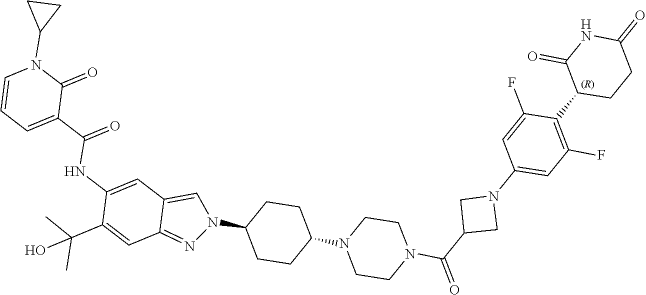

To a solution of 1-cyclopropyl-N-(6-(2-hydroxypropan-2-yl)-2-((1r,4r)-4-(piperazin-1-yl)cyclohexyl)-2H-indazol-5-yl)-2-oxo-1,2-dihydropyridine-3-carboxamide (41.44 mg, 0.08 mmol) and (R)-1-(4-(2,6-dioxopiperidin-3-yl)-3,5-difluorophenyl)azetidine-3-carboxylic acid (25.92 mg, 0.08 mmol) in DCM (5 mL) was added DIPEA (31 mg, 0.24 mmol) and T₃P in EtOAc (50%, 102 mg, 0.16 mmol) at rt. The resulting mixture was stirred for 2 hrs at rt. The mixture was diluted with water and extracted with DCM. The combined organic layer was dried over anhydrous Na₂SO₄, filtered and evaporated under vacuum. The residue was purified with Prep-HPLC (C-18 column chromatography (0.1% FA in water:acetonitrile=90:10˜60:40 gradient elution) to afford the desired product (20 mg, 30%). [M+H]⁺=825.4 ¹H NMR (500 MHz, DMSO) δ 12.04 (s, 1H), 10.86 (s, 1H), 8.38 (d, J=5.0 Hz, 1H), 8.28 (s, 1H), 8.16 (s, 1H), 7.92 (d, J=5.0 Hz, 1H), 7.58 (s, 1H), 6.46 (t, J=10.0 Hz, 2H), 6.18 (s, 1H), 6.16 (s, 1H), 5.40 (s, 1H), 4.46-4.38 (m, 1H), 4.07-4.02 (m, 3H), 3.94-3.89 (m, 2H), 3.86-3.80 (m, 1H), 3.52-3.45 (m, 3H), 3.29-3.27 (m, 2H), 2.81-2.73 (m, 1H), 2.56-2.51 (m, 2H), 2.49-2.41 (m, 4H), 2.20-2.01 (m, 3H), 2.00-1.89 (m, 5H), 1.59-1.41 (m, 8H), 1.10-1.03 (m, 2H), 0.97-0.92 (m, 2H).

### Example 103: 1-cyclopropyl-N-(2-((1R,4r)-4-(4-((R)-1-(4-((R)-2,6-dioxopiperidin-3-yl)-3,5-difluorophenyl)pyrrolidine-3-carbonyl)piperazin-1-yl)cyclohexyl)-6-(2-hydroxypropan-2-yl)-2H-indazol-5-yl)-2-oxo-1,2-dihydropyridine-3-carboxamide

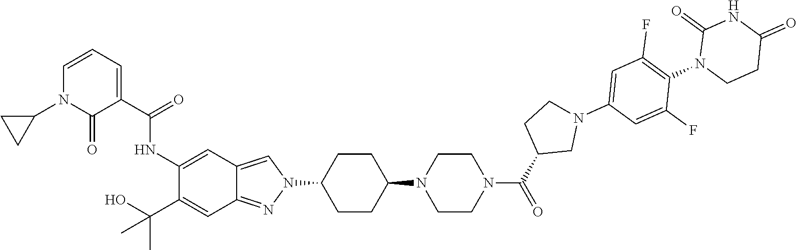

To a solution of 1-cyclopropyl-N-(6-(2-hydroxypropan-2-yl)-2-((1r,4r)-4-(piperazin-1-yl)cyclohexyl)-2H-indazol-5-yl)-2-oxo-1,2-dihydropyridine-3-carboxamide (20.72 mg, 0.04 mmol) and (R)-1-(4-((R)-2,6-dioxopiperidin-3-yl)-3,5-difluorophenyl)pyrrolidine-3-carboxylic acid (13.52 mg, 0.04 mmol) in DCM (5 mL) was added DIPEA (15.48 mg, 0.12 mmol) and T₃P in EtOAc (50%, 50.78 mg, 0.08 mmol) at rt. The resulting mixture was stirred for 2 hrs at rt. The mixture was diluted with water and extracted with DCM. The combined organic layer was dried over anhydrous Na₂SO₄, filtered and evaporated under vacuum. The residue was purified with Prep-HPLC (C-18 column chromatography (0.1% FA in water:acetonitrile=90:10˜60:40 gradient elution) to yield the desired product (9.75 mg, 30.4%). [M+H]⁺=839.4 ¹H NMR (500 MHz, DMSO) δ 12.05 (s, 1H), 10.85 (s, 1H), 9.65 (s, 1H), 8.38 (d, J=5.0 Hz, 1H), 8.31 (s, 1H), 8.18 (s, 1H), 7.93 (d, J=5.0 Hz, 1H), 7.58 (s, 1H), 6.47 (t, J=10.0 Hz, 2H), 6.26 (s, 1H), 6.23 (s, 1H), 5.42 (s, 1H), 4.58-4.50 (m, 2H), 4.31-4.24 (m, 1H), 4.02 (dd, J=10.0 Hz, 5.0 Hz, 1H), 3.61-3.40 (m, 8H), 3.29-3.18 (m, 4H), 3.15-2.97 (m, 2H), 2.82-2.71 (m, 1H), 2.33-1.90 (m, 10H), 1.80-1.70 (m, 2H), 1.57 (s, 6H), 1.10-1.05 (m, 2H), 0.97-0.93 (m, 2H).

### Example 117: (R)-1-cyclopropyl-N-(2-(1′-(1-(4-(2,6-dioxopiperidin-3-yl)-3,5-difluorophenyl)azetidine-3-carbonyl)-[1,4′-bipiperidin]-4-yl)-6-(2-hydroxypropan-2-yl)-2H-indazol-5-yl)-2-oxo-1,2-dihydropyridine-3-carboxamide

### Step 1: methyl 2-(1′-(tert-butoxycarbonyl)-[1,4′-bipiperidin]-4-yl)-5-(1-cyclopropyl-2-oxo-1,2-dihydropyridine-3-carboxamido)-2H-indazole-6-carboxylate

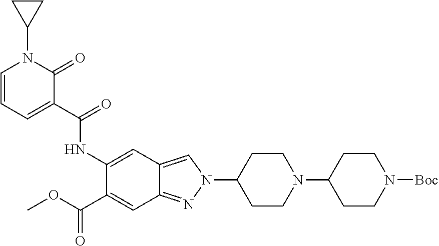 [SMILES: COC(=O)c1cc2nn(C3CCN(C4CCN(C(=O)OC(C)(C)C)CC4)CC3)cc2cc1NC(=O)c1cccn(C2CC2)c1=O]

To a solution of methyl 5-amino-2-(1′-(tert-butoxycarbonyl)-[1,4′-bipiperidin]-4-yl)-2H-indazole-6-carboxylate (0.4 g, 0.875 mmol) and 1-cyclopropyl-2-oxo-1,2-dihydropyridine-3-carboxylic acid (0.157 g, 0.875 mmol) in DMF (10 mL) was added DIPEA (0.339 g, 2.625 mmol) and HATU (0.332 g, 0.875 mmol) at rt. The resulting mixture was stirred for 2 hrs at rt. The mixture was diluted with water and extracted with DCM. The combined organic layer was dried over anhydrous Na₂SO₄, filtered and evaporated under vacuum. The residue was purified with silica gel column chromatography (DCM:MeOH=20:1) to afford product (0.41 g, 75.8%). [M+H]⁺=619.3

### Step 2: tert-butyl 4-(5-(1-cyclopropyl-2-oxo-1,2-dihydropyridine-3-carboxamido)-6-(2-hydroxypropan-2-yl)-2H-indazol-2-yl)-[1,4′-bipiperidine]-1′-carboxylate

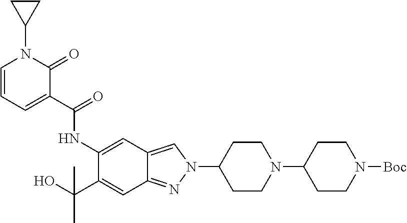 [SMILES: CC(C)(C)OC(=O)N1CCC(N2CCC(n3cc4cc(NC(=O)c5cccn(C6CC6)c5=O)c(C(C)(C)O)cc4n3)CC2)CC1]

To a solution of methyl 2-(1′-(tert-butoxycarbonyl)-[1,4′-bipiperidin]-4-yl)-5-(1-cyclopropyl-2-oxo-1,2-dihydropyridine-3-carboxamido)-2H-indazole-6-carboxylate (0.41 g, 0.663 mmol) and LiCl (0.141 g, 3.315 mmol) in THF (10 mL) was added dropwise MeMgBr in THF (1 M, 6.63 mL, 6.63 mmol) at 0° C. The resulting mixture was stirred for 2 hrs at 0° C. The mixture was quenched with sat.NH₄Cl solution and extracted with DCM. The combined organic layer was dried over anhydrous Na₂SO₄, filtered and evaporated under vacuum. The residue was purified with silica gel column chromatography (DCM:MeOH=20:1) to afford product (0.18 g, 43.9%). [M+H]⁺=619.4

### Step 3: N-(2-([1,4′-bipiperidin]-4-yl)-6-(2-hydroxypropan-2-yl)-2H-indazol-5-yl)-1-cyclopropyl-2-oxo-1,2-dihydropyridine-3-carboxamide

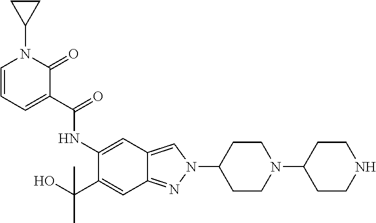 [SMILES: CC(C)(O)c1cc2nn(C3CCN(C4CCNCC4)CC3)cc2cc1NC(=O)c1cccn(C2CC2)c1=O]

To a solution of tert-butyl 4-(5-(1-cyclopropyl-2-oxo-1,2-dihydropyridine-3-carboxamido)-6-(2-hydroxypropan-2-yl)-2H-indazol-2-yl)-[1,4′-bipiperidine]-1′-carboxylate (0.18 g, 0.291 mmol) in DCM (10 mL) was added TFA (2 mL) at rt. The resulting mixture was stirred for 1 h at rt. The mixture was quenched with sat.NaHCO₃ solution and extracted with DCM. The combined organic layer was dried over anhydrous Na₂SO₄, filtered and evaporated under vacuum to afford product (0.11 g, 73.3%). [M+H]⁺=519.3

### Step 4: (R)-1-cyclopropyl-N-(2-(1′-(1-(4-(2,6-dioxopiperidin-3-yl)-3,5-difluorophenyl)azetidine-3-carbonyl)-[1,4′-bipiperidin]-4-yl)-6-(2-hydroxypropan-2-yl)-2H-indazol-5-yl)-2-oxo-1,2-dihydropyridine-3-carboxamide

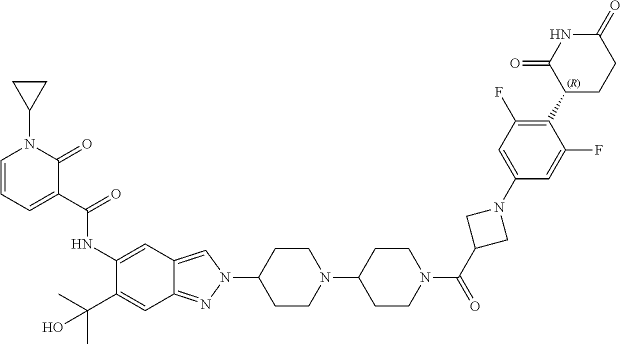

To a solution of N-(2-([1,4′-bipiperidin]-4-yl)-6-(2-hydroxypropan-2-yl)-2H-indazol-5-yl)-1-cyclopropyl-2-oxo-1,2-dihydropyridine-3-carboxamide (41 mg, 0.08 mmol) and (R)-1-(4-(2,6-dioxopiperidin-3-yl)-3,5-difluorophenyl)azetidine-3-carboxylic acid (26 mg, 0.08 mmol) in DCM (5 mL) was added DIPEA (31 mg, 0.24 mmol) and T₃P in EtOAc (50%, 102 mg, 0.16 mmol) at rt. The resulting mixture was stirred for 2 hrs at rt. The mixture was diluted with water and extracted with DCM. The combined organic layer was dried over anhydrous Na₂SO₄, filtered and evaporated under vacuum. The residue was purified with Prep-HPLC (C-18 column chromatography (0.1% FA in water:acetonitrile=90:10˜60:40 gradient elution) to afford the desired product (32.08 mg, 48.7%). [M+H]⁺=825.4 ¹H NMR (500 MHz, DMSO) δ 12.05 (s, 1H), 10.85 (s, 1H), 8.38 (d, J=5.0 Hz, 1H), 8.33 (s, 1H), 8.16 (s, 1H), 7.93 (d, J=5.0 Hz, 1H), 7.58 (s, 1H), 6.46 (t, J=10.0 Hz, 2H), 6.18 (s, 1H), 6.16 (s, 1H), 5.40 (s, 1H), 4.45-4.38 (m, 2H), 4.07-4.00 (m, 3H), 3.95-3.89 (m, 2H), 3.85-3.80 (m, 1H), 3.68-3.62 (m, 1H), 3.53-3.46 (m, 1H), 3.14-2.95 (m, 3H), 2.81-2.72 (m, 1H), 2.63-2.56 (m, 2H), 2.43-2.32 (m, 2H), 2.12-2.01 (m, 5H), 1.97-1.90 (m, 1H), 1.82-1.75 (m, 2H), 1.57 (s, 6H), 1.45-1.25 (m, 2H), 1.10-1.04 (m, 2H), 0.97-0.92 (m, 2H).

### Example 118: 1-cyclopropyl-N-(2-(1′-((R)-1-(4-((R)-2,6-dioxopiperidin-3-yl)-3,5-difluorophenyl)pyrrolidine-3-carbonyl)-[1,4′-bipiperidin]-4-yl)-6-(2-hydroxypropan-2-yl)-2H-indazol-5-yl)-2-oxo-1,2-dihydropyridine-3-carboxamide

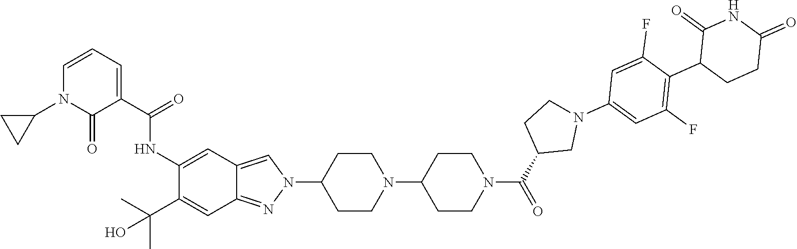 [SMILES: CC(C)(O)c1cc2nn(C3CCN(C4CCN(C(=O)[C@@H]5CCN(c6cc(F)c(C7CCC(=O)NC7=O)c(F)c6)C5)CC4)CC3)cc2cc1NC(=O)c1cccn(C2CC2)c1=O]

To a solution of N-(2-([1,4′-bipiperidin]-4-yl)-6-(2-hydroxypropan-2-yl)-2H-indazol-5-yl)-1-cyclopropyl-2-oxo-1,2-dihydropyridine-3-carboxamide (41 mg, 0.08 mmol) and (R)-1-(4-((R)-2,6-dioxopiperidin-3-yl)-3,5-difluorophenyl)pyrrolidine-3-carboxylic acid (27 mg, 0.08 mmol) in DCM (5 mL) was added DIPEA (31 mg, 0.24 mmol) and T₃P in EtOAc (50%, 102 mg, 0.16 mmol) at rt. The resulting mixture was stirred for 2 hrs at rt. The mixture was diluted with water and extracted with DCM. The combined organic layers were dried over anhydrous Na₂SO₄, filtered and evaporated under vacuum. The residue was purified with Prep-HPLC (C-18 column chromatography (0.1% FA in water:acetonitrile=90:10˜60:40 gradient elution) to afford the desired product (20.81 mg, 31%). [M+H]⁺=839.4 ¹H NMR (500 MHz, DMSO) δ 12.04 (s, 1H), 10.84 (s, 1H), 8.38 (d, J=5.0 Hz, 1H), 8.33 (s, 1H), 8.16 (s, 1H), 7.92 (d, J=5.0 Hz, 1H), 7.58 (s, 1H), 6.46 (t, J=10.0 Hz, 2H), 6.23 (s, 1H), 6.20 (s, 1H), 5.41 (s, 1H), 4.44-4.38 (m, 2H), 4.10-3.98 (m, 2H), 3.95-3.89 (m, 2H), 3.85-3.80 (m, 1H), 3.68-3.62 (m, 1H), 3.53-3.46 (m, 1H), 3.14-2.95 (m, 3H), 2.81-2.72 (m, 1H), 2.63-2.56 (m, 2H), 2.48-2.33 (m, 4H), 2.20-2.01 (m, 7H), 1.97-1.90 (m, 1H), 1.87-1.77 (m, 2H), 1.57 (s, 6H), 1.53-1.40 (m, 1H), 1.33-1.21 (m, 1H), 1.10-1.02 (m, 2H), 0.97-0.92 (m, 2H).

### Example 160: (R)—N-(2-(1′-(1-(4-(2,6-dioxopiperidin-3-yl)-3,5-difluorophenyl)azetidine-3-carbonyl)-[1,4′-bipiperidin]-4-yl)-6-(2-hydroxypropan-2-yl)-2H-indazol-5-yl)-6-(trifluoromethyl)picolinamide

### Step 1: tert-butyl 4-(6-bromo-2H-indazol-2-yl)-[1,4′-bipiperidine]-1′-carboxylate

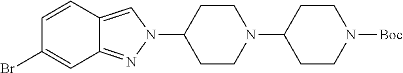 [SMILES: CC(C)(C)OC(=O)N1CCC(N2CCC(n3cc4ccc(Br)cc4n3)CC2)CC1]

To a solution of 4-bromo-2-nitrobenzaldehyde (2.3 g, 10 mmol) in propan-2-ol (50 mL) was added tert-butyl 4-amino-[1,4′-bipiperidine]-1′-carboxylate (2.83 g, 10 mmol). The mixture was stirred for 3 hrs at 80° C. under N₂. To the above solution was added tributylphosphane (6.06 g, 30 mmol). The mixture was stirred overnight at 80° C. under N₂. The mixture was cooled to rt and concentrated in vacuo. The residue was purified by silica gel column chromatography (DCM:MeOH=30:1) to give product (3.5 g, 76%). [M+H]⁺=463.2

### Step 2: methyl 2-(1′-(tert-butoxycarbonyl)-[1,4′-bipiperidin]-4-yl)-2H-indazole-6-carboxylate

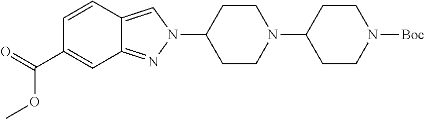 [SMILES: COC(=O)c1ccc2cn(C3CCN(C4CCN(C(=O)OC(C)(C)C)CC4)CC3)nc2c1]

To a solution of tert-butyl 4-(6-bromo-2H-indazol-2-yl)-[1,4′-bipiperidine]-1′-carboxylate (3.5 g, 7.58 mmol) and Et₃N (2.3 g, 22.74 mmol) in MeOH (40 mL) was added Pd(dppf)Cl₂ (0.555 g, 0.758 mmol) at rt. The mixture was stirred overnight at 80° C. under CO (g). The mixture was concentrated under vacuum. The residue was purified with silica gel column chromatography (DCM:MeOH=30:1) to afford product (2.5 g, 75%). [M+H]⁺=443.3

### Step 3: methyl 2-([1,4′-bipiperidin]-4-yl)-5-nitro-2H-indazole-6-carboxylate

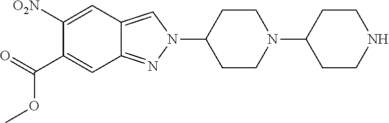 [SMILES: COC(=O)c1cc2nn(C3CCN(C4CCNCC4)CC3)cc2cc1[N+](=O)[O-]]

To a solution of methyl 2-(1′-(tert-butoxycarbonyl)-[1,4′-bipiperidin]-4-yl)-2H-indazole-6-carboxylate (2.5 g, 5.66 mmol) in conc.H₂SO₄ (30 mL) was slowed added NaNO₃ (0.577 g, 6.792 mmol) at 0° C. The mixture was stirred for 2 hrs at 0° C. The mixture was quenched with ice-water, then adjusted to pH 9 with sat.Na₂CO₃ solution and extracted with DCM. The combined organic layer was dried over anhydrous Na₂SO₄, filtered and evaporated under vacuum to afford product (1.5 g, 68.5%), which was used without further purification. [M+H]⁺=388.2

### Step 4: methyl 2-(1′-(tert-butoxycarbonyl)-[1,4′-bipiperidin]-4-yl)-5-nitro-2H-indazole-6-carboxylate

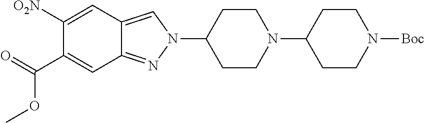 [SMILES: COC(=O)c1cc2nn(C3CCN(C4CCN(C(=O)OC(C)(C)C)CC4)CC3)cc2cc1[N+](=O)[O-]]

To a stirred solution of methyl 2-([1,4′-bipiperidin]-4-yl)-5-nitro-2H-indazole-6-carboxylate (1.5 g, 3.88 mmol) in DCM (20 mL) was added Et₃N (1.176 g, 11.64 mmol) and Boc₂O (1.269 g, 5.82 mmol). The reaction mixture was stirred overnight at rt. The mixture was concentrated under vacuum. The residue was purified with silica gel column chromatography (DCM:MeOH=30:1) to afford product (1.6 g, 84.5%). [M+H]⁺=488.2

### Step 5: methyl 5-amino-2-(1′-(tert-butoxycarbonyl)-[1,4′-bipiperidin]-4-yl)-2H-indazole-6-carboxylate

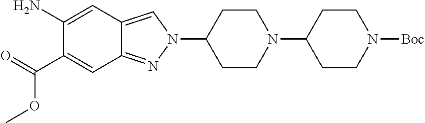 [SMILES: COC(=O)c1cc2nn(C3CCN(C4CCN(C(=O)OC(C)(C)C)CC4)CC3)cc2cc1N]

To a solution of methyl 2-(1′-(tert-butoxycarbonyl)-[1,4′-bipiperidin]-4-yl)-5-nitro-2H-indazole-6-carboxylate (1.6 g, 3.29 mmol) in MeOH (20 mL) and water (4 mL) was added NH₄Cl (0.88 g, 16.45 mmol) and Fe powder (0.92 g, 16.45 mmol) at room temperature. The resulting mixture was stirred for 2 hrs at 70° C. The mixture was cooled to rt and concentrated under vacuum. The residue was purified with silica gel column chromatography (DCM:MeOH=15:1) to afford product (0.9 g, 60%). [M+H]⁺=458.3

### Step 6: methyl 2-(1′-(tert-butoxycarbonyl)-[1,4′-bipiperidin]-4-yl)-5-(6-(trifluoromethyl)picolinamido)-2H-indazole-6-carboxylate

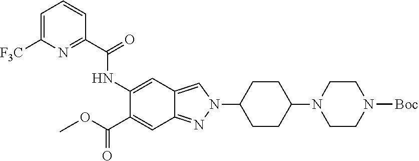 [SMILES: COC(=O)c1cc2nn(C3CCC(N4CCN(C(=O)OC(C)(C)C)CC4)CC3)cc2cc1NC(=O)c1cccc(C(F)(F)F)n1]

To a solution of methyl 5-amino-2-(1′-(tert-butoxycarbonyl)-[1,4′-bipiperidin]-4-yl)-2H-indazole-6-carboxylate (0.4 g, 0.875 mmol) and 6-(trifluoromethyl)picolinic acid (0.167 g, 0.875 mmol) in DMF (10 mL) was added DIPEA (0.339 g, 2.625 mmol) and HATU (0.332 g, 0.875 mmol) at rt. The resulting mixture was stirred for 2 hrs at rt. The mixture was diluted with water and extracted with DCM. The combined organic layer was dried over anhydrous Na₂SO₄, filtered and evaporated under vacuum. The residue was purified with silica gel column chromatography (DCM:MeOH=20:1) to afford product (0.4 g, 72.6%). [M+H]⁺=631.3

### Step 7: tert-butyl 4-(6-(2-hydroxypropan-2-yl)-5-(6-(trifluoromethyl)picolinamido)-2H-indazol-2-yl)-[1,4′-bipiperidine]-1′-carboxylate

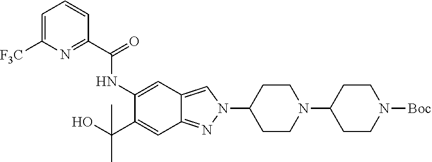 [SMILES: CC(C)(C)OC(=O)N1CCC(N2CCC(n3cc4cc(NC(=O)c5cccc(C(F)(F)F)n5)c(C(C)(C)O)cc4n3)CC2)CC1]

To a solution of methyl 2-(1′-(tert-butoxycarbonyl)-[1,4′-bipiperidin]-4-yl)-5-(6-(trifluoromethyl)picolinamido)-2H-indazole-6-carboxylate (0.4 g, 0.635 mmol) and LiCl (0.135 g, 3.175 mmol) in THF (10 mL) was added dropwise MeMgBr in THF (1 M, 6.4 mL, 6.4 mmol) at 0° C. The resulting mixture was stirred for 2 hrs at 0° C. The mixture was quenched with sat.NH₄Cl solution and extracted with DCM. The combined organic layer was dried over anhydrous Na₂SO₄, filtered and evaporated under vacuum. The residue was purified with silica gel column chromatography (DCM:MeOH=20:1) to afford product (0.24 g, 60%). [M+H]⁺=631.3

### Step 8: N-(2-([1,4′-bipiperidin]-4-yl)-6-(2-hydroxypropan-2-yl)-2H-indazol-5-yl)-6-(trifluoromethyl)picolinamide

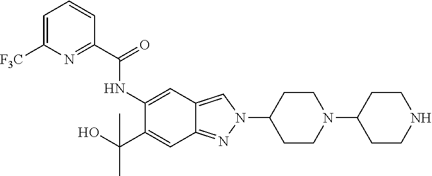 [SMILES: CC(C)(O)c1cc2nn(C3CCN(C4CCNCC4)CC3)cc2cc1NC(=O)c1cccc(C(F)(F)F)n1]

To a solution of tert-butyl 4-(6-(2-hydroxypropan-2-yl)-5-(6-(trifluoromethyl)picolinamido)-2H-indazol-2-yl)-[1,4′-bipiperidine]-1′-carboxylate (0.24 g, 0.381 mmol) in DCM (10 mL) was added TFA (2 mL) at rt. The resulting mixture was stirred for 1 h at rt. The mixture was quenched with sat.NaHCO₃ solution and extracted with DCM. The combined organic layers were dried over anhydrous Na₂SO₄, filtered and evaporated under vacuum to afford product (0.17 g, 85%). [M+H]⁺=531.3

### Step 9: (R)—N-(2-(1′-(1-(4-(2,6-dioxopiperidin-3-yl)-3,5-difluorophenyl)azetidine-3-carbonyl)-[1,4′-bipiperidin]-4-yl)-6-(2-hydroxypropan-2-yl)-2H-indazol-5-yl)-6-(trifluoromethyl)picolinamide

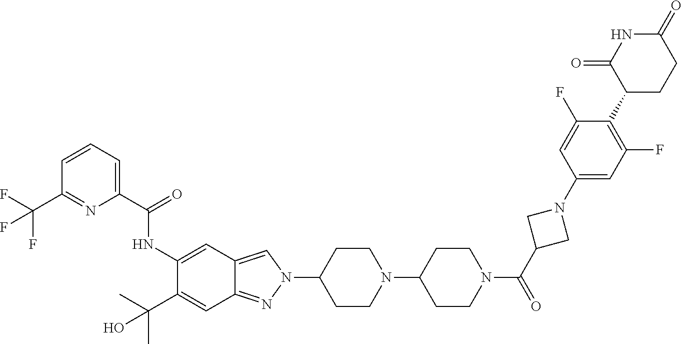 [SMILES: CC(C)(O)c1cc2nn(C3CCN(C4CCN(C(=O)C5CN(c6cc(F)c([C@H]7CCC(=O)NC7=O)c(F)c6)C5)CC4)CC3)cc2cc1NC(=O)c1cccc(C(F)(F)F)n1]

To a solution of N-(2-([1,4′-bipiperidin]-4-yl)-6-(2-hydroxypropan-2-yl)-2H-indazol-5-yl)-6-(trifluoromethyl)picolinamide (21 mg, 0.04 mmol) and (R)-1-(4-(2,6-dioxopiperidin-3-yl)-3,5-difluorophenyl)azetidine-3-carboxylic acid (13 mg, 0.04 mmol) in DCM (5 mL) was added DIPEA (15 mg, 0.12 mmol) and T₃P in EtOAc (50%, 51 mg, 0.08 mmol) at rt. The resulting mixture was stirred for 2 hrs at rt. The mixture was diluted with water and extracted with DCM. The combined organic layer was dried over anhydrous Na₂SO₄, filtered and evaporated under vacuum. The residue was purified with prep-HPLC (C-18 column chromatography (0.1% FA in water:acetonitrile=90:10˜60:40 gradient elution) to afford the desired product (7.27 mg, 22%). [M+H]⁺=837.3 ¹H NMR (500 MHz, DMSO) δ 12.37 (s, 1H), 10.86 (s, 1H), 8.72 (s, 1H), 8.45 (d, J=10.0 Hz, 1H), 8.40 (s, 1H), 8.38 (t, J=5.0 Hz, 1H), 8.16 (d, J=10.0 Hz, 1H), 7.57 (s, 1H), 6.18 (s, 1H), 6.16 (s, 1H), 5.94 (s, 1H), 4.44-4.38 (m, 2H), 4.10-3.98 (m, 3H), 3.95-3.89 (m, 2H), 3.85-3.79 (m, 1H), 3.68-3.62 (m, 1H), 3.04-2.95 (m, 3H), 2.81-2.72 (m, 1H), 2.64-2.55 (m, 3H), 2.45-2.32 (m, 2H), 2.23-2.01 (m, 5H), 1.97-1.90 (m, 1H), 1.82-1.75 (m, 2H), 1.62 (s, 6H), 1.44-1.24 (m, 2H).

### Example 161: N-(2-(1′-((R)-1-(4-((R)-2,6-dioxopiperidin-3-yl)-3,5-difluorophenyl)pyrrolidine-3-carbonyl)-[1,4′-bipiperidin]-4-yl)-6-(2-hydroxypropan-2-yl)-2H-indazol-5-yl)-6-(trifluoromethyl)picolinamide

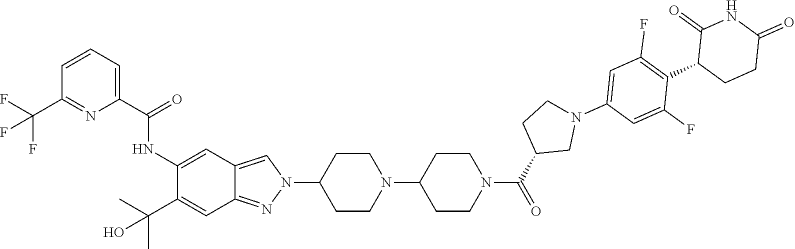 [SMILES: CC(C)(O)c1cc2nn(C3CCN(C4CCN(C(=O)[C@@H]5CCN(c6cc(F)c([C@H]7CCC(=O)NC7=O)c(F)c6)C5)CC4)CC3)cc2cc1NC(=O)c1cccc(C(F)(F)F)n1]

To a solution of N-(2-([1,4′-bipiperidin]-4-yl)-6-(2-hydroxypropan-2-yl)-2H-indazol-5-yl)-6-(trifluoromethyl)picolinamide (42 mg, 0.08 mmol) and (R)-1-(4-((R)-2,6-dioxopiperidin-3-yl)-3,5-difluorophenyl)pyrrolidine-3-carboxylic acid (27 mg, 0.08 mmol) in DCM (5 mL) was added DIPEA (31 mg, 0.24 mmol) and T₃P in EtOAc (50%, 102 mg, 0.16 mmol) at rt. The resulting mixture was stirred for 2 hrs at rt. The mixture was diluted with water and extracted with DCM. The combined organic layers were dried over anhydrous Na₂SO₄, filtered and evaporated under vacuum. The residue was purified with prep-HPLC (C-18 column chromatography (0.1% FA in water:acetonitrile=90:10˜60:40 gradient elution) to afford the desired product (27 mg, 39%). [M+H]⁺=851.4 ¹H NMR (500 MHz, DMSO) δ 12.37 (s, 1H), 10.84 (s, 1H), 8.72 (s, 1H), 8.45 (d, J=10.0 Hz, 1H), 8.40 (s, 1H), 8.37 (t, J=5.0 Hz, 1H), 8.16 (d, J=10.0 Hz, 1H), 7.57 (s, 1H), 6.23 (t, J=10.0 Hz, 2H), 5.94 (s, 1H), 4.44-4.38 (m, 2H), 4.10-3.98 (m, 2H), 3.58-3.50 (m, 1H), 3.48-3.40 (m, 1H), 3.36-3.31 (m, 2H), 3.29-3.21 (m, 2H), 3.10-2.98 (m, 3H), 2.81-2.72 (m, 1H), 2.64-2.55 (m, 2H), 2.44-2.35 (m, 2H), 2.20-2.01 (m, 7H), 1.97-1.90 (m, 1H), 1.87-1.76 (m, 2H), 1.62 (s, 6H), 1.52-1.38 (m, 1H), 1.35-1.22 (m, 1H).

### Example 162: (R)—N-(2-(1′-(1-(4-(2,4-dioxotetrahydropyrimidin-1(2H)-yl)phenyl)pyrrolidine-3-carbonyl)-[1,4′-bipiperidin]-4-yl)-6-(2-hydroxypropan-2-yl)-2H-indazol-5-yl)-6-(trifluoromethyl)picolinamide

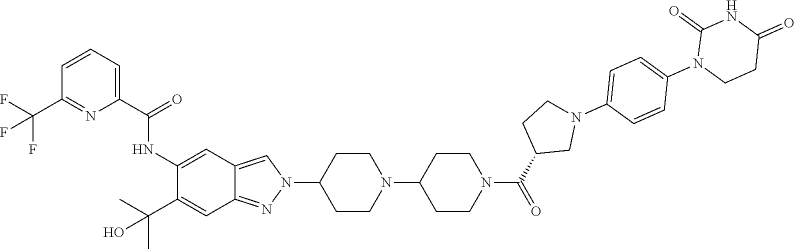 [SMILES: CC(C)(O)c1cc2nn(C3CCN(C4CCN(C(=O)[C@@H]5CCN(c6ccc(N7CCC(=O)NC7=O)cc6)C5)CC4)CC3)cc2cc1NC(=O)c1cccc(C(F)(F)F)n1]

To a solution of N-(2-([1,4′-bipiperidin]-4-yl)-6-(2-hydroxypropan-2-yl)-2H-indazol-5-yl)-6-(trifluoromethyl)picolinamide (42 mg, 0.08 mmol) and (R)-1-(4-(2,4-dioxotetrahydropyrimidin-1(2H)-yl)phenyl)pyrrolidine-3-carboxylic acid (obtained in a manner similar to Common intermediate B) (24 mg, 0.08 mmol) in DCM (5 mL) was added DIPEA (31 mg, 0.24 mmol) and T₃P in EtOAc (50%, 102 mg, 0.16 mmol) at rt. The resulting mixture was stirred for 2 hrs at rt. The mixture was diluted with water and extracted with DCM. The combined organic layer was dried over anhydrous Na₂SO₄, filtered and evaporated under vacuum. The residue was purified with prep-HPLC (C-18 column chromatography (0.1% FA in water:acetonitrile=90:10˜60:40 gradient elution) to afford the desired product (25.18 mg, 39%). [M+H]⁺=816.4 ¹H NMR (500 MHz, DMSO) δ 12.37 (s, 1H), 10.21 (s, 1H), 8.72 (s, 1H), 8.45 (d, J=10.0 Hz, 1H), 8.40 (s, 1H), 8.37 (t, J=5.0 Hz, 1H), 8.16 (d, J=10.0 Hz, 1H), 7.58 (s, 1H), 7.10 (d, J=10.0 Hz, 2H), 6.54 (t, J=10.0 Hz, 2H), 5.94 (s, 1H), 4.44-4.38 (m, 2H), 4.14-4.05 (m, 1H), 3.67 (t, J=10.0 Hz, 2H), 3.58-3.42 (m, 2H), 3.29-3.23 (m, 2H), 3.11-2.98 (m, 3H), 2.69-2.55 (m, 4H), 2.44-2.35 (m, 3H), 2.21-2.01 (m, 6H), 1.89-1.78 (m, 2H), 1.62 (s, 6H), 1.52-1.26 (m, 2H).

### Example 163: (S)—N-(2-(1′-(1-(4-(2,4-dioxotetrahydropyrimidin-1(2H)-yl)phenyl)pyrrolidine-3-carbonyl)-[1,4′-bipiperidin]-4-yl)-6-(2-hydroxypropan-2-yl)-2H-indazol-5-yl)-6-(trifluoromethyl)picolinamide

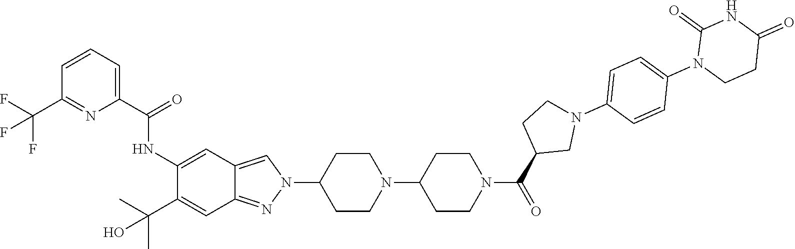 [SMILES: CC(C)(O)c1cc2nn(C3CCN(C4CCN(C(=O)[C@H]5CCN(c6ccc(N7CCC(=O)NC7=O)cc6)C5)CC4)CC3)cc2cc1NC(=O)c1cccc(C(F)(F)F)n1]

To a solution of N-(2-([1,4′-bipiperidin]-4-yl)-6-(2-hydroxypropan-2-yl)-2H-indazol-5-yl)-6-(trifluoromethyl)picolinamide (42 mg, 0.08 mmol) and (S)-1-(4-(2,4-dioxotetrahydropyrimidin-1(2H)-yl)phenyl)pyrrolidine-3-carboxylic acid (obtained in a manner similar to Common intermediate B) (24 mg, 0.08 mmol) in DCM (5 mL) was added DIPEA (31 mg, 0.24 mmol) and T₃P in EtOAc (50%, 102 mg, 0.16 mmol) at rt. The resulting mixture was stirred for 2 hrs at rt. The mixture was diluted with water and extracted with DCM. The combined organic layers were dried over anhydrous Na₂SO₄, filtered and evaporated in vacuum. The residue was purified with prep-HPLC (C-18 column chromatography (0.1% FA in water:acetonitrile=90:10˜60:40 gradient elution) to afford the desired product (32.95 mg, 51%). [M+H]⁺=816.4 ¹H NMR (500 MHz, DMSO) δ 12.37 (s, 1H), 10.21 (s, 1H), 8.72 (s, 1H), 8.45 (d, J=10.0 Hz, 1H), 8.40 (s, 1H), 8.37 (t, J=5.0 Hz, 1H), 8.16 (d, J=10.0 Hz, 1H), 7.58 (s, 1H), 7.10 (d, J=10.0 Hz, 2H), 6.54 (t, J=10.0 Hz, 2H), 5.94 (s, 1H), 4.44-4.38 (m, 2H), 4.14-4.05 (m, 1H), 3.67 (t, J=10.0 Hz, 2H), 3.58-3.42 (m, 2H), 3.38-3.31 (m, 1H), 3.29-3.23 (m, 2H), 3.11-2.98 (m, 3H), 2.69-2.55 (m, 4H), 2.44-2.35 (m, 2H), 2.21-2.01 (m, 6H), 1.89-1.78 (m, 2H), 1.62 (s, 6H), 1.50-1.25 (m, 2H).

### Example 178: 3-cyano-N-(2-((1r,4r)-4-(4-(1-(4-((R)-2,6-dioxopiperidin-3-yl)-3,5-difluorophenyl)azetidine-3-carbonyl)piperazin-1-yl)cyclohexyl)-6-(2-hydroxypropan-2-yl)-2H-indazol-5-yl)pyrrolo[1,2-b]pyridazine-7-carboxamide

### Step 1: 3,3-diethoxy-2-formylpropanenitrile potassium salt

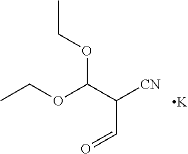

To a stirred solution of 3,3-diethoxypropane-nitrile (14.3 g, 100 mmol) and methyl formate (7.5 g, 125 mmol) in anhydrous THF (100 mL) at 10° C. was added 1.0 M potassium tert-butoxide in THF (110 ml, 110 mmol). The temperature was maintained between 10° C. to 15° C. during the 45 minutes addition. The resulting slurry was stirred for 2 hours at ambient temperature afterwards. Hexane (400 mL) was then added and the mixture was stirred for another 20 min. The slurry was filtered and the filter cake washed with 1:1 hexanes/THF and dried under vacuum to provide the product (16 g, 76 mmol, 76%).

### Step 2: pyrrolo[1,2-b]pyridazine-3-carbonitrile

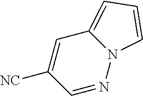 [SMILES: N#Cc1cnn2cccc2c1]

A stirring suspension of 3,3-diethoxy-2-formylpropanenitrile potassium salt (9 g, 42.85 mmol) in MeOH (80 ml) was cooled to 0° C., and concentrated HCl (13.73 mL, 178.5 mmol) was added dropwise. After addition was complete, the reaction was stirred at room temperature for 20 minutes. To this reaction mixture was added a solution of 1-aminopyrrole (1.95 g, 23.80 mmol) in methanol (10 mL). After addition, the reaction mixture was refluxed and stirred at 90° C. for 2 hours, and cooled to room temperature and concentrated to about half of the original volume. Saturated aqueous sodium bicarbonate was added carefully to the resulting residue until bubbling stopped. The solution was extracted with two portions of ethyl acetate. The combined organic layer was dried over sodium sulfate, filtered, concentrated under vacuum. The resulting residue was purified with silica gel chromatography (PE:EA=3:1) to provide the product (2.1 g, 14.6 mmol, 61.7%). [M+H]⁺=144.6.

### Step 3: 7-bromopyrrolo[1,2-b]pyridazine-3-carbonitrile

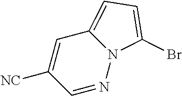 [SMILES: N#Cc1cnn2c(Br)ccc2c1]

To a solution of pyrrolo[1,2-b]pyridazine-3-carbonitrile (2.8 g, 19.6 mmol) in MeCN (50 mL) at room temperature was added N-bromosuccinimide (3.48 g, 19.6 mmol) in one portion. The reaction was stirred at room temperature for 30 minutes then poured into saturated aqueous sodium bicarbonate. The solution was concentrated under vacuum to remove the acetonitrile. The resulting aqueous layer was extracted with three portions of EtOAc. The combined organic layer was dried over sodium sulfate, filtered, concentrated under vacuum, and purified with silica gel chromatography (PE:DCM=7:3) to provide the product (1.5 g, 6.75 mmol, 34%) [M+H]⁺=222.6.

### Step 4: 3-cyanopyrrolo[1,2-b]pyridazine-7-carboxylic acid

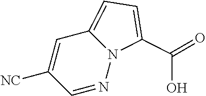 [SMILES: N#Cc1cnn2c(C(=O)O)ccc2c1]

To a high pressure reactor was added a suspension of 7-bromopyrrolo[1,2-b]pyridazine-3-carbonitrile (500 mg, 2.25 mmol), Pd(dppf)Cl₂ (79.86 mg, 0.11 mmol), Pd(OAc)₂ (25.28 mg, 0.11 mmol), Xantphos (130 mg, 0.23 mmol) and TEA (682 mg, 6.75 mmol) in THF/H₂O (30 mL/10 mL). The reactor was stirred for 15 h at 100° C. under the atmosphere of CO (40 atm). Then the suspension was concentrated under vacuum. The residue was diluted with water (30 mL) and acidified with HCl (2N in water) to pH=3. Then the mixture was extracted with DCM/i-PrOH=3/1 (50 mL×3). The organic layer was combined, dried over anhydrous Na₂SO₄, and concentrated in vacuo. The residue was purified with Combi-Flash (silica column, 12 g, DCM:MeOH=10:1) to yield the product (250 mg, 1.33 mmol, 59%). [M+H]⁺=188.6.

### Step 5: tert-butyl 4-((1r,4r)-4-(5-(3-cyanopyrrolo[1,2-b]pyridazine-7-carboxamido)-6-(2-hydroxypropan-2-yl)-2H-indazol-2-yl)cyclohexyl)piperazine-1-carboxylate

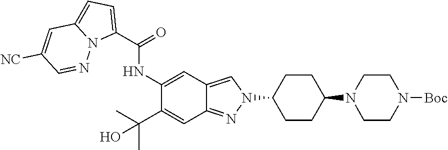 [SMILES: CC(C)(C)OC(=O)N1CCN([C@H]2CC[C@H](n3cc4cc(NC(=O)c5ccc6cc(C#N)cnn56)c(C(C)(C)O)cc4n3)CC2)CC1]

To a stirred solution of 3-cyanopyrrolo[1,2-b]pyridazine-7-carboxylic acid (30 mg, 0.16 mmol) and tert-butyl 4-((1r,4r)-4-(5-amino-6-(2-hydroxypropan-2-yl)-2H-indazol-2-yl)cyclohexyl)piperazine-1-carboxylate (66 mg, 0.15 mmol) in DMF (3 mL) was added HATU (66 mg, 0.17 mmol) and DIEA (29 mg, 0.45 mmol). The solution was stirred at rt for 4 h. Then the solution was diluted with EA (20 mL), washed with water and brine. The organic layer was dried over Na₂SO₄ and concentrated under vacuum. The residue was purified with Combi-Flash (silica column, 4 g, DCM:MeOH=20:1) to yield the product (55 mg, 0.09 mmol, 58%). [M+H]⁺=627.6.

### Step 6: 3-cyano-N-(6-(2-hydroxypropan-2-yl)-2-((1r,4r)-4-(piperazin-1-yl)cyclohexyl)-2H-indazol-5-yl)pyrrolo[1,2-b]pyridazine-7-carboxamide

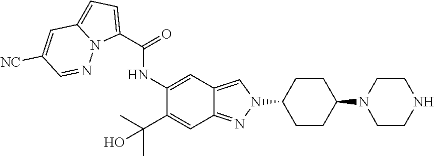 [SMILES: CC(C)(O)c1cc2nn([C@H]3CC[C@H](N4CCNCC4)CC3)cc2cc1NC(=O)c1ccc2cc(C#N)cnn12]

To a stirring solution of tert-butyl 4-((1r,4r)-4-(5-(3-cyanopyrrolo[1,2-b]pyridazine-7-carboxamido)-6-(2-hydroxypropan-2-yl)-2H-indazol-2-yl)cyclohexyl)piperazine-1-carboxylate (55 mg, 0.09 mmol) in DCM (3 mL) was added TFA (1 mL). The reaction mixture was stirred at rt for 2 h and concentrated under vacuum to afford the crude product (60 mg). [M+H]⁺=527.6.

### Step 7: 3-cyano-N-(2-((1r,4r)-4-(4-(1-(4-((R)-2,6-dioxopiperidin-3-yl)-3,5-difluorophenyl)azetidine-3-carbonyl)piperazin-1-yl)cyclohexyl)-6-(2-hydroxypropan-2-yl)-2H-indazol-5-yl)pyrrolo[1,2-b]pyridazine-7-carboxamide

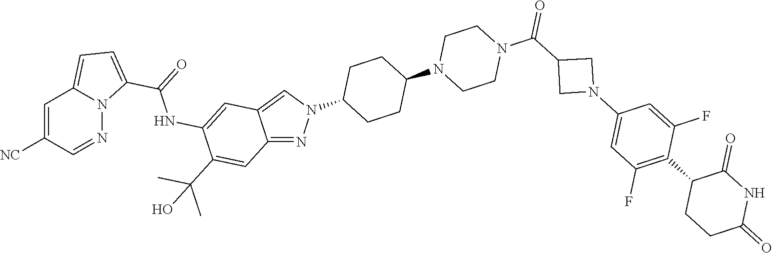 [SMILES: CC(C)(O)c1cc2nn([C@H]3CC[C@H](N4CCN(C(=O)C5CN(c6cc(F)c([C@H]7CCC(=O)NC7=O)c(F)c6)C5)CC4)CC3)cc2cc1NC(=O)c1ccc2cc(C#N)cnn12]

To a solution of crude 3-cyano-N-(6-(2-hydroxypropan-2-yl)-2-((1r,4r)-4-(piperazin-1-yl)cyclohexyl)-2H-indazol-5-yl)pyrrolo[1,2-b]pyridazine-7-carboxamide (60 mg, 0.09 mmol), (R)-1-(4-(2,6-dioxopiperidin-3-yl)-3,5-difluorophenyl)azetidine-3-carboxylic acid (32.4 mg, 0.10 mmol) and DIEA (35 mg, 0.272 mmol) in DCM (3 mL) was added T₃P (72 mg, 0.227 mmol) dropwise at room temperature. The resulting mixture was stirred at room temperature for 3 hour. The reaction was quenched with water (20 mL) and extracted with DCM (2×10 mL). The combined organic layers were dried over anhydrous Na₂SO₄, filtered and evaporated in vacuum to afford the crude residue, which was purified by silica gel column chromatography (DCM:MeOH=100:0˜10:1 gradient elution) to afford product, which was further purified with prep-HPLC (C-18 column chromatography (0.1% FA in water:acetonitrile=90:10˜60:40 gradient elution) to yield the desired product (23.5 mg, 31%).

¹H NMR (500 MHz, DMSO) δ 12.02 (s, 1H), 10.86 (s, 1H), 8.93 (d, J=2.0 Hz, 1H), 8.74 (d, J=2.0 Hz, 1H), 8.55 (s, 1H), 8.34 (s, 1H), 7.72 (d, J=4.7 Hz, 1H), 7.57 (s, 1H), 7.09 (d, J=4.7 Hz, 1H), 6.17 (d, J=11.1 Hz, 2H), 5.72 (s, 1H), 4.43 (t, J=11.4 Hz, 1H), 4.10-4.00 (m, 3H), 3.92 (t, J=6.3 Hz, 2H), 3.86-3.79 (m, 1H), 3.49 (s, 2H), 2.83-2.73 (m, 1H), 2.58-2.44 (m, 8H), 2.18 (d, J=8.8 Hz, 2H), 2.09-2.03 (m, 1H), 1.99-1.94 (m, 5H), 1.63 (s, 6H), 1.50 (q, J=11.8 Hz, 2H). [M+H]⁺=833.5.

### Example 105: 3-cyano-N-(2-((1r,4r)-4-(4-(1-(4-(2,4-dioxotetrahydropyrimidin-1(2H)-yl)-3,5-difluorophenyl)azetidine-3-carbonyl)piperazin-1-yl)cyclohexyl)-6-(2-hydroxypropan-2-yl)-2H-indazol-5-yl)pyrrolo[1,2-b]pyridazine-7-carboxamide

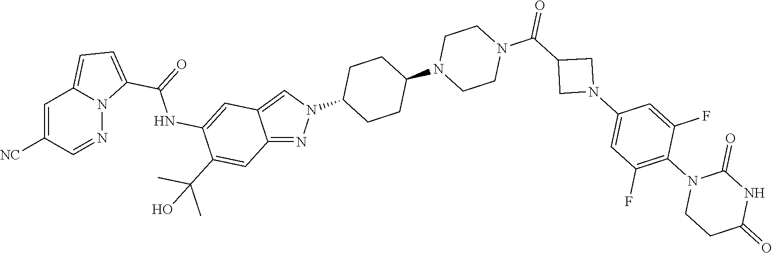 [SMILES: CC(C)(O)c1cc2nn([C@H]3CC[C@H](N4CCN(C(=O)C5CN(c6cc(F)c(N7CCC(=O)NC7=O)c(F)c6)C5)CC4)CC3)cc2cc1NC(=O)c1ccc2cc(C#N)cnn12]

The title compound (15 mg, 23.7%) was prepared in a manner similar to that described in Example 178.

¹H NMR (500 MHz, DMSO) δ 12.02 (s, 1H), 10.50 (s, 1H), 8.93 (d, J=2.0 Hz, 1H), 8.74 (d, J=2.0 Hz, 1H), 8.55 (s, 1H), 8.34 (s, 1H), 7.72 (d, J=4.5 Hz, 1H), 7.57 (s, 1H), 7.09 (d, J=4.5 Hz, 1H), 6.25 (d, J=10.0 Hz, 2H), 5.73 (s, 1H), 4.48-4.35 (m, 1H), 4.06 (t, J=8.0 Hz, 2H), 3.95 (t, J=6.5 Hz, 2H), 3.88-3.82 (m, 1H), 3.59 (t, J=6.6 Hz, 2H), 3.51-3.46 (m, 2H), 2.68 (t, J=6.6 Hz, 2H), 2.60-2.52 (m, 5H), 2.41-2.34 (m, 2H), 2.22-2.15 (m, 2H), 1.99-1.92 (m, 4H), 1.63 (s, 6H), 1.53-1.47 (m, 2H). [M+H]+=834.5.

### Example 110: 3-cyano-N-(2-((1R,4r)-4-(4-(1-(4-((R)-2,6-dioxopiperidin-3-yl)-3,5-difluorophenyl)azetidin-3-yl)piperazin-1-yl)cyclohexyl)-6-(2-hydroxypropan-2-yl)-2H-indazol-5-yl)pyrrolo[1,2-b]pyridazine-7-carboxamide

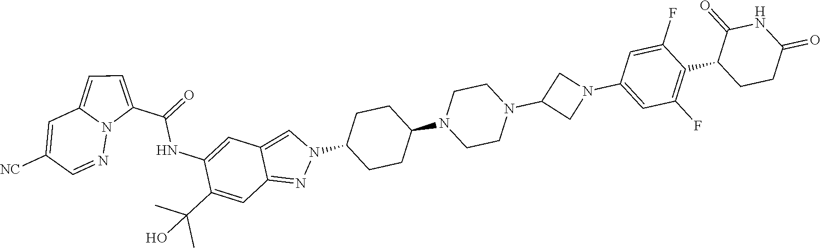 [SMILES: CC(C)(O)c1cc2nn([C@H]3CC[C@H](N4CCN(C5CN(c6cc(F)c([C@H]7CCC(=O)NC7=O)c(F)c6)C5)CC4)CC3)cc2cc1NC(=O)c1ccc2cc(C#N)cnn12]

The title compound (3 mg, 3.2%) was prepared in a manner similar to that described in Example 9. ¹H NMR (500 MHz, DMSO) δ 12.02 (s, 1H), 10.86 (s, 1H), 8.93 (s, 1H), 8.74 (s, 1H), 8.55 (s, 1H), 8.33 (s, 1H), 7.71 (s, 1H), 7.57 (s, 1H), 7.09 (s, 1H), 6.12 (d, J=11.5 Hz, 2H), 5.72 (s, 1H), 4.45-4.38 (m, 1H), 4.21 (s, 1H), 4.05-4.01 (m, 1H), 3.93-3.87 (m, 2H), 3.66-3.61 (m, 2H), 3.25-3.23 (m, 2H), 2.80-2.75 (m, 1H), 2.60-2.55 (s, 4H), 2.36 (s, 4H), 2.25 (s, 1H), 2.19-2.14 (s, 2H), 2.06 (s, 1H), 1.97-1.91 (m, 4H), 1.63 (s, 6H), 1.53-1.45 (m, 3H). [M+H]⁺=805.7.

This compound could also be synthesized in another manner.

### Step 1: methyl 2-((1r,4r)-4-(4-(tert-butoxycarbonyl)piperazin-1-yl)cyclohexyl)-2H-indazole-6-carboxylate

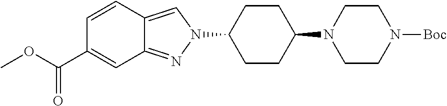 [SMILES: COC(=O)c1ccc2cn([C@H]3CC[C@H](N4CCN(C(=O)OC(C)(C)C)CC4)CC3)nc2c1]

To a solution of methyl 4-formyl-3-nitrobenzoate (104.5 g, 500 mmol) in propan-2-ol (1000 mL) was added tert-butyl 4-((1r,4r)-4-aminocyclohexyl)piperazine-1-carboxylate (141.5 g, 500 mmol). The mixture was stirred for 3 hrs at 80° C. under N₂. To the above solution was added tri-n-butylphosphane (303 g, 1500 mmol). The mixture was stirred overnight at 80° C. under N₂. The mixture was cooled to room temperature and concentrated in vacuo. The residue was slurried with petroleum ether (3000 mL) and filtered. The solid was filtered and dried under vacuum to yield product (160 g, 72.4%). [M+H]⁺=443.1

### Step 2: methyl 5-nitro-2-((1r,4r)-4-(piperazin-1-yl)cyclohexyl)-2H-indazole-6-carboxylate

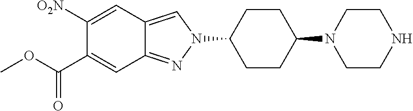 [SMILES: COC(=O)c1cc2nn([C@H]3CC[C@H](N4CCNCC4)CC3)cc2cc1[N+](=O)[O-]]

To a solution of methyl 2-((1r,4r)-4-(4-(tert-butoxycarbonyl)piperazin-1-yl)cyclohexyl)-2H-indazole-6-carboxylate (160 g, 362 mmol) in conc.H₂SO₄ (98%, 1000 mL) was slowed added NaNO₃ (46.155 g, 543 mmol) at 0° C. The mixture was stirred for 2 hrs at 0° C. The mixture was quenched with ice-water, then adjusted to pH 9 with sat.Na₂CO₃ solution and extracted with DCM (3×2000 mL). The combined organic layers were dried over anhydrous Na₂SO₄, filtered and evaporated in vacuum to yield product (140 g, crude), which was used without further purification. [M+H]⁺=388.1

### Step 3: methyl 2-((1r,4r)-4-(4-(tert-butoxycarbonyl)piperazin-1-yl)cyclohexyl)-5-nitro-2H-indazole-6-carboxylate

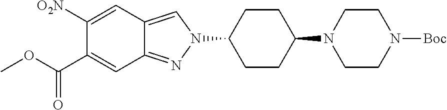 [SMILES: COC(=O)c1cc2nn([C@H]3CC[C@H](N4CCN(C(=O)OC(C)(C)C)CC4)CC3)cc2cc1[N+](=O)[O-]]

To a stirred solution of methyl 5-nitro-2-((1r,4r)-4-(piperazin-1-yl)cyclohexyl)-2H-indazole-6-carboxylate (140 g crude, 362 mmol) in DCM (1200 mL) was added Et₃N (109.686 g, 1086 mmol) and Boc₂O (94.7 g, 434.4 mmol). The reaction mixture was stirred overnight at room temperature. The mixture was concentrated in vacuo. The residue was purified with silica gel column chromatography (DCM:MeOH=30:1) to yield product (150 g, 84.9%). [M+H]⁺=488.1

### Step 4: methyl 5-amino-2-((1r,4r)-4-(4-(tert-butoxycarbonyl)piperazin-1-yl)cyclohexyl)-2H-indazole-6-carboxylate

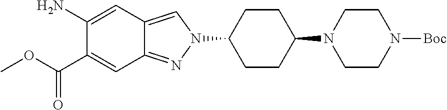 [SMILES: COC(=O)c1cc2nn([C@H]3CC[C@H](N4CCN(C(=O)OC(C)(C)C)CC4)CC3)cc2cc1N]

To a solution of methyl 2-((1r,4r)-4-(4-(tert-butoxycarbonyl)piperazin-1-yl)cyclohexyl)-5-nitro-2H-indazole-6-carboxylate (150 g, 308 mmol) in THF (1500 mL) was added Pd/C (5%, 30 g) at room temperature. The resulting mixture was stirred overnight at room temperature under H₂. The solid was filtered and washed with MeOH (800 mL). The filtrate was evaporated in vacuum to yield product (120 g, 85.2%). [M+H]⁺=458.2

### Step 5: methyl 2-((1r,4r)-4-(4-(tert-butoxycarbonyl)piperazin-1-yl)cyclohexyl)-5-(2,2,2-trifluoroacetamido)-2H-indazole-6-carboxylate

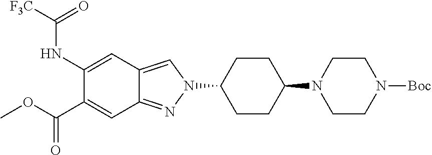 [SMILES: COC(=O)c1cc2nn([C@H]3CC[C@H](N4CCN(C(=O)OC(C)(C)C)CC4)CC3)cc2cc1NC(=O)C(F)(F)F]

To a solution of methyl 5-amino-2-((1r,4r)-4-(4-(tert-butoxycarbonyl)piperazin-1-yl)cyclohexyl)-2H-indazole-6-carboxylate (120 g, 262.58 mmol) in DCM (1000 mL) was added dropwise trifluoroacetic anhydride (66.15 g, 315 mmol) at 0° C. The mixture was stirred for 2 hrs at 0° C. The mixture was quenched with ice-water, then adjusted to pH 8 with sat.NaHCO₃ solution and extracted with DCM (3^{×}1000 mL). The combined organic layers were dried over anhydrous Na₂SO₄, filtered and evaporated in vacuum. The residue was purified with silica gel column chromatography (DCM:MeOH=30:1) to yield product (132 g, 90.9%). [M+H]⁺=554.2

### Step 6: tert-butyl 4-((1r,4r)-4-(6-(2-hydroxypropan-2-yl)-5-(2,2,2-trifluoroacetamido)-2H-indazol-2-yl)cyclohexyl)piperazine-1-carboxylate

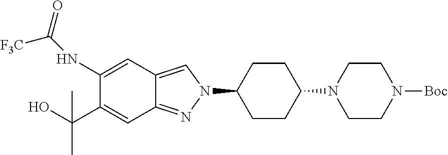 [SMILES: CC(C)(C)OC(=O)N1CCN([C@H]2CC[C@H](n3cc4cc(NC(=O)C(F)(F)F)c(C(C)(C)O)cc4n3)CC2)CC1]

To a solution of methyl 2-((1r,4r)-4-(4-(tert-butoxycarbonyl)piperazin-1-yl)cyclohexyl)-5-(2,2,2-trifluoroacetamido)-2H-indazole-6-carboxylate (132 g, 238.698 mmol) and LiCl (50.723 g, 1193.49 mmol) in THF (1500 mL) was added dropwise MeMgBr in THF (3 M, 795.66 mL, 2386.98 mmol) at 0° C. The resulting mixture was stirred overnight at 0° C. The mixture was quenched with sat.NH₄Cl solution and extracted with EtOAc (3^{×}1000 mL). The combined organic layer was dried over anhydrous Na₂SO₄, filtered and concentrated under vacuum. The residue was purified with silica gel column chromatography (DCM:MeOH=20:1) to yield product (105 g, 79.55%). [M+H]⁺=554.2

### Step 7: tert-butyl 4-((1r,4r)-4-(5-amino-6-(2-hydroxypropan-2-yl)-2H-indazol-2-yl)cyclohexyl)piperazine-1-carboxylate

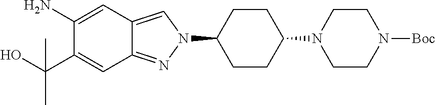 [SMILES: CC(C)(C)OC(=O)N1CCN([C@H]2CC[C@H](n3cc4cc(N)c(C(C)(C)O)cc4n3)CC2)CC1]

To a solution of tert-butyl 4-((1r,4r)-4-(6-(2-hydroxypropan-2-yl)-5-(2,2,2-trifluoroacetamido)-2H-indazol-2-yl)cyclohexyl)piperazine-1-carboxylate (105 g, 189.87 mmol) in MeOH (1000 mL) was added NaOH solution (2 M, 950 mL, 1898.7 mmol) at room temperature. The mixture was stirred for 2 hrs at 70° C. The mixture was cooled to room temperature, diluted with water (600 mL) and extracted with DCM/MeOH (V/V=10/1, 591000 mL). The combined organic layers were dried over anhydrous Na₂SO₄, filtered and evaporated in vacuum. The residue was slurried with petroleum ether (1000 mL) and filtered. The solid was filtered and dried under vacuum to yield product (80 g, 92.2%). [M+H]⁺=458.2

### Step 8: tert-butyl 4-((1r,4r)-4-(5-(3-cyanopyrrolo[1,2-b]pyridazine-7-carboxamido)-6-(2-hydroxypropan-2-yl)-2H-indazol-2-yl)cyclohexyl)piperazine-1-carboxylate

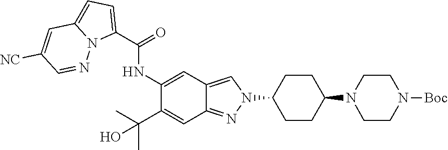 [SMILES: CC(C)(C)OC(=O)N1CCN([C@H]2CC[C@H](n3cc4cc(NC(=O)c5ccc6cc(C#N)cnn56)c(C(C)(C)O)cc4n3)CC2)CC1]

To a stirred solution of 3-cyanopyrrolo[1,2-b]pyridazine-7-carboxylic acid (36 g, 192.56 mmol) and tert-butyl 4-((1r,4r)-4-(5-amino-6-(2-hydroxypropan-2-yl)-2H-indazol-2-yl)cyclohexyl)piperazine-1-carboxylate (80 g, 175.05 mmol) in DMF (1000 mL) was added HATU (79.822 g, 210.06 mmol) and DIEA (67.744 g, 525.15 mmol). The mixture was stirred for 3 hrs at room temperature. The mixture was diluted with water (3000 mL). The solid was collected by filtration and washed with water (3^{×}100 mL). The solid was dried under vacuum to yield product (90 g, 82.13%). [M+H]⁺=627.5

### Step 9: 3-cyano-N-(6-(2-hydroxypropan-2-yl)-2-((1r,4r)-4-(piperazin-1-yl)cyclohexyl)-2H-indazol-5-yl)pyrrolo[1,2-b]pyridazine-7-carboxamide

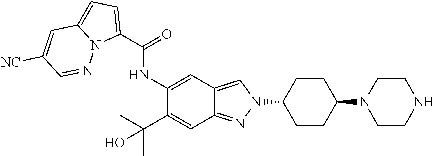 [SMILES: CC(C)(O)c1cc2nn([C@H]3CC[C@H](N4CCNCC4)CC3)cc2cc1NC(=O)c1ccc2cc(C#N)cnn12]

To a stirring solution of tert-butyl 4-((1r,4r)-4-(5-(3-cyanopyrrolo[1,2-b]pyridazine-7-carboxamido)-6-(2-hydroxypropan-2-yl)-2H-indazol-2-yl)cyclohexyl)piperazine-1-carboxylate (90 g, 143.77 mmol) in MeOH (600 mL) was added dropwise conc.HCl (36%, 300 mL) at 0° C. The resulting mixture was stirred overnight at room temperature. The solid was collected by filtration. The solid was dissolve with water (500 mL), then adjusted to pH 8 with sat.NaHCO₃ solution and extracted with DCM/MeOH (V/V=10/1, 5^{×}2000 mL). The combined organic layers were dried over anhydrous Na₂SO₄, filtered and evaporated in vacuum. The residue was slurried with petroleum ether (1000 mL) and filtered. The solid was filtered and dried under vacuum to yield product (64 g, 84.63%). [M+H]⁺=527.4

### Step 10: 3-cyano-N-(2-((1R,4r)-4-(4-(1-(4-((R)-2,6-dioxopiperidin-3-yl)-3,5-difluorophenyl)azetidin-3-yl)piperazin-1-yl)cyclohexyl)-6-(2-hydroxypropan-2-yl)-2H-indazol-5-yl)pyrrolo[1,2-b]pyridazine-7-carboxamide

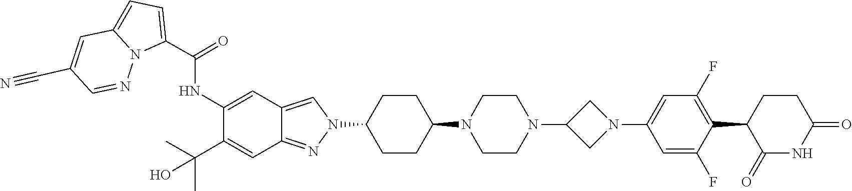 [SMILES: CC(C)(O)c1cc2nn([C@H]3CC[C@H](N4CCN(C5CN(c6cc(F)c([C@H]7CCC(=O)NC7=O)c(F)c6)C5)CC4)CC3)cc2cc1NC(=O)c1ccc2cc(C#N)cnn12]

To a stirred solution of 3-cyano-N-(6-(2-hydroxypropan-2-yl)-2-((1r,4r)-4-(piperazin-1-yl)cyclohexyl)-2H-indazol-5-yl)pyrrolo[1,2-b]pyridazine-7-carboxamide (60 g, 114.07 mmol) and (R)-3-(2,6-difluoro-4-(3-oxoazetidin-1-yl)phenyl)piperidine-2,6-dione (50.3 g, 171.1 mmol) in DCE (1000 mL) was added AcOH (6.844 g, 114.07 mmol) and NaBH(OAc)₃ (48.366 g, 228.14 mmol). The mixture was stirred overnight at room temperature. The mixture was diluted with MeOH (500 mL) and concentrated in vacuo. The residue was purified with silica gel column chromatography (DCM:MeOH=15:1). After, add acetonitrile (1600 mL), water (800 mL) and acetic acid (200 mL) to freeze dry to yield product (66 g, 62.62%).

### Example 181: 3-cyano-N-(2-((1R,4r)-4-(4-((R)-1-(4-((R)-2,6-dioxopiperidin-3-yl)-3,5-difluorophenyl)pyrrolidine-3-carbonyl)piperazin-1-yl)cyclohexyl)-6-(2-hydroxypropan-2-yl)-2H-indazol-5-yl)pyrrolo[1,2-b]pyridazine-7-carboxamide

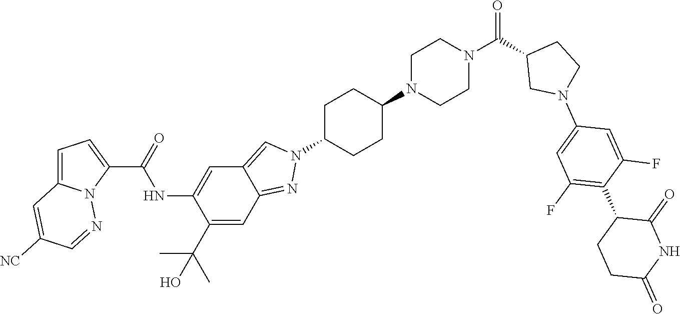 [SMILES: CC(C)(O)c1cc2nn([C@H]3CC[C@H](N4CCN(C(=O)[C@@H]5CCN(c6cc(F)c([C@H]7CCC(=O)NC7=O)c(F)c6)C5)CC4)CC3)cc2cc1NC(=O)c1ccc2cc(C#N)cnn12]

The title compound (45 mg, 46.6%) was prepared in a manner similar to that described in Example 178.

¹H NMR (500 MHz, DMSO) δ 12.02 (s, 1H), 10.84 (s, 1H), 8.93 (d, J=2.0 Hz, 1H), 8.74 (d, J=2.0 Hz, 1H), 8.55 (s, 1H), 8.34 (s, 1H), 7.72 (d, J=4.5 Hz, 1H), 7.57 (s, 1H), 7.09 (d, J=4.5 Hz, 1H), 6.23 (d, J=12.0 Hz, 2H), 5.72 (s, 1H), 4.47-4.44 (m, 1H), 4.02 (dd, J=12.5, 5.0 Hz, 1H), 3.60-3.42 (m, 7H), 3.30-3.20 (m, 4H), 2.82-2.74 (m, 1H), 2.61-2.55 (m, 2H), 2.20-2.15 (m, 3H), 2.11-2.05 (m, 2H), 2.00-1.89 (m, 6H), 1.63 (s, 6H), 1.57-1.44 (m, 3H). [M+H]⁺=847.5.

### Example 182: 3-cyano-N-(2-((1R,4r)-4-(4-(4-((R)-2,6-dioxopiperidin-3-yl)-3,5-difluorophenethyl)piperazin-1-yl)cyclohexyl)-6-(2-hydroxypropan-2-yl)-2H-indazol-5-yl)pyrrolo[1,2-b]pyridazine-7-carboxamide

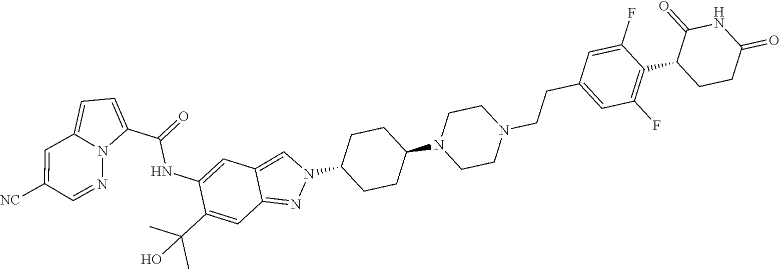 [SMILES: CC(C)(O)c1cc2nn([C@H]3CC[C@H](N4CCN(CCc5cc(F)c([C@H]6CCC(=O)NC6=O)c(F)c5)CC4)CC3)cc2cc1NC(=O)c1ccc2cc(C#N)cnn12]

The title compound (18 mg, 32.9%) was prepared in a manner similar to that described in Example 9. ¹H NMR (500 MHz, DMSO) δ 12.02 (s, 1H), 10.94 (s, 1H), 8.93 (d, J=2.0 Hz, 1H), 8.74 (d, J=2.0 Hz, 1H), 8.55 (s, 1H), 8.33 (s, 1H), 8.14 (s, 1H), 7.72 (d, J=4.5 Hz, 1H), 7.57 (s, 1H), 7.09 (d, J=4.5 Hz, 1H), 7.03 (d, J=10.0 Hz, 2H), 5.71 (s, 1H), 4.46-4.38 (m, 1H), 4.23-4.17 (m, 1H), 2.81-2.75 (m, 3H), 2.65-2.53 (m, 7H), 2.36-2.33 (m, 1H), 2.20-2.10 (m, 4H), 2.01-1.90 (m, 6H), 1.63 (s, 6H), 1.54-1.45 (m, 3H). [M+H]⁺=778.7.

This compound could also be synthesized in another manner.

### Step 1: methyl 2-((1r,4r)-4-(4-(tert-butoxycarbonyl)piperazin-1-yl)cyclohexyl)-2H-indazole-6-carboxylate

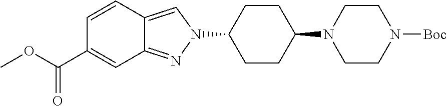 [SMILES: COC(=O)c1ccc2cn([C@H]3CC[C@H](N4CCN(C(=O)OC(C)(C)C)CC4)CC3)nc2c1]

To a solution of methyl 4-formyl-3-nitrobenzoate (104.5 g, 500 mmol) in propan-2-ol (1000 mL) was added tert-butyl 4-((1r,4r)-4-aminocyclohexyl)piperazine-1-carboxylate (141.5 g, 500 mmol). The mixture was stirred for 3 hrs at 80° C. under N₂. Then the mixture was cooled to rt and tri-n-butylphosphane (303 g, 1500 mmol) was added slowly. The mixture was stirred overnight at 80° C. under N₂. The mixture was cooled to room temperature and concentrated in vacuo. The residue was slurried with petroleum ether (3000 mL) and filtered. The solid was filtered and dried under vacuum to yield product (130 g, 58.7%). [M+H]⁺=443.1

### Step 2: methyl 5-nitro-2-((1r,4r)-4-(piperazin-1-yl)cyclohexyl)-2H-indazole-6-carboxylate

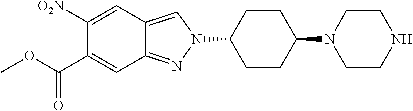 [SMILES: COC(=O)c1cc2nn([C@H]3CC[C@H](N4CCNCC4)CC3)cc2cc1[N+](=O)[O-]]

To a solution of methyl 2-((1r,4r)-4-(4-(tert-butoxycarbonyl)piperazin-1-yl)cyclohexyl)-2H-indazole-6-carboxylate (130 g, 293.7 mmol) in conc.H₂SO₄ (98%, 1000 mL) was added NaNO₃ (49.9 g, 587.4 mmol) in portions at 0° C. The mixture was stirred for 2 hrs at 0° C. Then the mixture was pour into ice-water, The mixture was alkalinized to pH 9 with saturated Na₂CO₃ aqueous solution and extracted with DCM (3×2000 mL). The combined organic layers were dried over anhydrous Na₂SO₄, filtered and concentrated in vacuum to yield product (130 g, crude), which was directly used in next step without any further purification. [M+H]⁺=388.1

### Step 3: methyl 2-((1r,4r)-4-(4-(tert-butoxycarbonyl)piperazin-1-yl)cyclohexyl)-5-nitro-2H-indazole-6-carboxylate

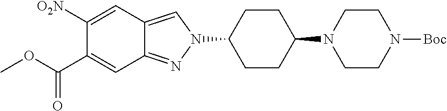 [SMILES: COC(=O)c1cc2nn([C@H]3CC[C@H](N4CCN(C(=O)OC(C)(C)C)CC4)CC3)cc2cc1[N+](=O)[O-]]

To a stirred solution of methyl 5-nitro-2-((1r,4r)-4-(piperazin-1-yl)cyclohexyl)-2H-indazole-6-carboxylate (140 g crude) in DCM (1000 mL) was added Et₃N (44.50 g, 440.55 mmol) and Boc₂O (64.1 g, 293.7 mmol). The reaction mixture was stirred overnight at room temperature. The mixture was diluted with DCM (2000 mL) and washed with water (1 L×2) and saturated salt solution (1 L×2). Then the organic layer was dried over anhydrous Na2SO4 and filtered. The filtrate was concentrated in vacuo. The residue was purified with silica gel column chromatography (DCM:MeOH=30:1) to yield product (130 g, 90.8% over 2 steps). [M+H]⁺=488.1

### Step 4: methyl 5-amino-2-((1r,4r)-4-(4-(tert-butoxycarbonyl)piperazin-1-yl)cyclohexyl)-2H-indazole-6-carboxylate

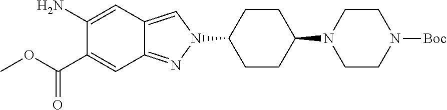 [SMILES: COC(=O)c1cc2nn([C@H]3CC[C@H](N4CCN(C(=O)OC(C)(C)C)CC4)CC3)cc2cc1N]

To a solution of methyl 2-((1r,4r)-4-(4-(tert-butoxycarbonyl)piperazin-1-yl)cyclohexyl)-5-nitro-2H-indazole-6-carboxylate (130 g, 266.6 mmol) in THF (1300 mL) was added Pd/C (5%, 30 g) at room temperature. The resulting mixture was stirred overnight at room temperature under H₂. The solid was filtered and washed with MeOH (800 mL). The filtrate was evaporated in vacuum to yield product (110 g, 90.3%). [M+H]⁺=458.2

### Step 5: methyl 2-((1r,4r)-4-(4-(tert-butoxycarbonyl)piperazin-1-yl)cyclohexyl)-5-(2,2,2-trifluoroacetamido)-2H-indazole-6-carboxylate

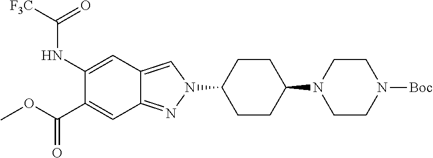 [SMILES: COC(=O)c1cc2nn([C@H]3CC[C@H](N4CCN(C(=O)OC(C)(C)C)CC4)CC3)cc2cc1NC(=O)C(F)(F)F]

To a solution of methyl 5-amino-2-((1r,4r)-4-(4-(tert-butoxycarbonyl)piperazin-1-yl)cyclohexyl)-2H-indazole-6-carboxylate (105 g, 229.4 mmol) in DCM (1000 mL) was added dropwise trifluoroacetic anhydride (50.6 g, 240.9 mmol) at 0° C. The mixture was stirred for 2 hrs at 0° C. The mixture was quenched with ice-water, then adjusted to pH 8 with sat.NaHCO₃ solution and extracted with DCM (3^{×}1000 mL). The combined organic layers were dried over anhydrous Na₂SO₄, filtered and evaporated in vacuum. The residue was purified with silica gel column chromatography (DCM:MeOH=30:1) to yield product (120 g, 94.5%). [M+H]⁺=554.2

### Step 6: tert-butyl 4-((1r,4r)-4-(6-(2-hydroxypropan-2-yl)-5-(2,2,2-trifluoroacetamido)-2H-indazol-2-yl)cyclohexyl)piperazine-1-carboxylate

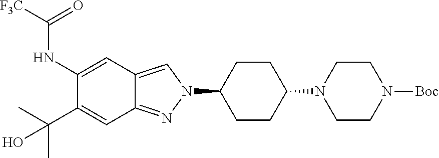 [SMILES: CC(C)(C)OC(=O)N1CCN([C@H]2CC[C@H](n3cc4cc(NC(=O)C(F)(F)F)c(C(C)(C)O)cc4n3)CC2)CC1]

To a solution of methyl 2-((1r,4r)-4-(4-(tert-butoxycarbonyl)piperazin-1-yl)cyclohexyl)-5-(2,2,2-trifluoroacetamido)-2H-indazole-6-carboxylate (115 g, 207.7 mmol) and LiCl (44.03 g, 1038.7 mmol) in THF (1500 mL) was added dropwise MeMgBr in THF (3 M, 692.4 mL, 2077.2 mmol) at 0° C. under N₂. The resulting mixture was stirred overnight at 0° C. The mixture was quenched slowly with sat.NH₄Cl solution (1500 mL) and acidified to PH 5 with HCl (1 mol/L). Then the mixture was extracted with EtOAc (3×1000 mL). The combined organic layer was dried over anhydrous Na₂SO₄, filtered and concentrated in vacuo. The residue was purified with silica gel column chromatography (DCM:MeOH=20:1) to yield product (100 g, 86.9%). [M+H]⁺=554.2

### Step 7: tert-butyl 4-((1r,4r)-4-(5-amino-6-(2-hydroxypropan-2-yl)-2H-indazol-2-yl)cyclohexyl)piperazine-1-carboxylate

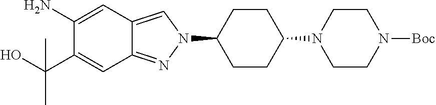 [SMILES: CC(C)(C)OC(=O)N1CCN([C@H]2CC[C@H](n3cc4cc(N)c(C(C)(C)O)cc4n3)CC2)CC1]

To a solution of tert-butyl 4-((1r,4r)-4-(6-(2-hydroxypropan-2-yl)-5-(2,2,2-trifluoroacetamido)-2H-indazol-2-yl)cyclohexyl)piperazine-1-carboxylate (100 g, 180.62 mmol) in MeOH (1000 mL) was added NaOH solution (2 N, 903 mL, 1806.2 mmol) at room temperature. The mixture was stirred for 2 hrs at 70° C. The mixture was cooled to room temperature and solid was precipitated. The mixture was filtered and the filter cake was washed with water. Then the filter cake was collected and dried under reduce pressure to give the product (75 g, 90.7%). [M+H]⁺=458.2

### Step 8: tert-butyl 4-((1r,4r)-4-(5-(3-cyanopyrrolo[1,2-b]pyridazine-7-carboxamido)-6-(2-hydroxypropan-2-yl)-2H-indazol-2-yl)cyclohexyl)piperazine-1-carboxylate

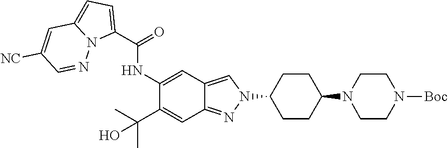 [SMILES: CC(C)(C)OC(=O)N1CCN([C@H]2CC[C@H](n3cc4cc(NC(=O)c5ccc6cc(C#N)cnn56)c(C(C)(C)O)cc4n3)CC2)CC1]

To a stirred solution of 3-cyanopyrrolo[1,2-b]pyridazine-7-carboxylic acid (36.81 g, 196.67 mmol) and tert-butyl 4-((1r,4r)-4-(5-amino-6-(2-hydroxypropan-2-yl)-2H-indazol-2-yl)cyclohexyl)piperazine-1-carboxylate (75 g, 163.89 mmol) in DMF (1000 mL) was added HATU (74.73 g, 196.67 mmol) and DIEA (42.28 g, 327.78 mmol). The mixture was stirred for 5 hrs at room temperature. The mixture was diluted with water (3000 mL). The solid was collected by filtration and washed with water (3^{×}100 mL). The solid was dried under vacuum to yield product (90 g, 87.62%). [M+H]⁺=627.5

### Step 9: 3-cyano-N-(6-(2-hydroxypropan-2-yl)-2-((1r,4r)-4-(piperazin-1-yl)cyclohexyl)-2H-indazol-5-yl)pyrrolo[1,2-b]pyridazine-7-carboxamide

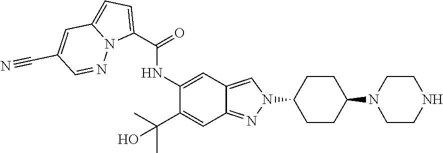 [SMILES: CC(C)(O)c1cc2nn([C@H]3CC[C@H](N4CCNCC4)CC3)cc2cc1NC(=O)c1ccc2cc(C#N)cnn12]

To a stirring solution of tert-butyl 4-((1r,4r)-4-(5-(3-cyanopyrrolo[1,2-b]pyridazine-7-carboxamido)-6-(2-hydroxypropan-2-yl)-2H-indazol-2-yl)cyclohexyl)piperazine-1-carboxylate (90 g, 143.77 mmol) in MeOH (600 mL) was added dropwise conc.HCl (36%, 300 mL) at 0° C. The resulting mixture was stirred overnight at room temperature. The solid was collected by filtration. The solid was dissolve with water (500 mL), then adjusted to pH 8 with sat.NaHCO₃ solution and extracted with DCM/MeOH (V/V=10/1, 5^{×}2000 mL). The combined organic layers were dried over anhydrous Na₂SO₄, filtered and evaporated in vacuum. The residue was slurried with petroleum ether (1000 mL) and filtered. The solid was filtered and dried under vacuum to yield product (70 g, 92.45%). [M+H]⁺=527.4

### Step 10: 3-cyano-N-(2-((1R,4r)-4-(4-(4-((R)-2,6-dioxopiperidin-3-yl)-3,5-difluorophenethyl)piperazin-1-yl)cyclohexyl)-6-(2-hydroxypropan-2-yl)-2H-indazol-5-yl)pyrrolo[1,2-b]pyridazine-7-carboxamide

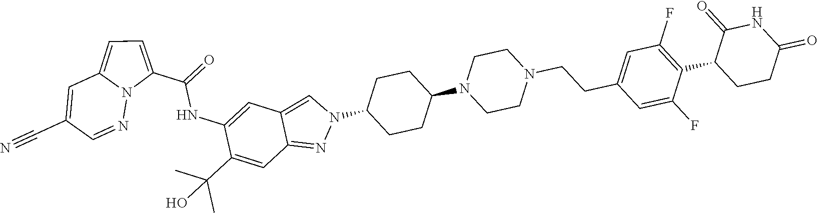 [SMILES: CC(C)(O)c1cc2nn([C@H]3CC[C@H](N4CCN(CCc5cc(F)c([C@H]6CCC(=O)NC6=O)c(F)c5)CC4)CC3)cc2cc1NC(=O)c1ccc2cc(C#N)cnn12]

To a stirred solution of 3-cyano-N-(6-(2-hydroxypropan-2-yl)-2-((1r,4r)-4-(piperazin-1-yl)cyclohexyl)-2H-indazol-5-yl)pyrrolo[1,2-b]pyridazine-7-carboxamide (4.9 g, 9.30 mmol) and (R)-2-(4-(2,6-dioxopiperidin-3-yl)-3,5-difluorophenyl)acetaldehyde (3.23 g, 12.09 mmol) (CAS: 2758533-79-6, which was obtained in the same way described in WO2022012622A1) in DCE (100 mL) was added AcOH (0.542 g, 9.30 mmol) and NaBH(OAc)₃ (2.96 g, 13.95 mmol). The mixture was stirred overnight at room temperature. The mixture was diluted with MeOH (30 mL) and concentrated in vacuo. The residue was purified with silica gel column chromatography (DCM:MeOH=15:1) to give the impure product. Then the impure product was slurried with MeOH (30 mL) and filtered. The filter cake was collected and dried under pressure to give the product (3.8 g, 52.53%). ¹H NMR (500 MHz, DMSO) δ 12.02 (s, 1H), 10.95 (s, 1H), 8.93 (d, J=2.2 Hz, 1H), 8.74 (d, J=2.2 Hz, 1H), 8.55 (s, 1H), 8.33 (s, 1H), 8.15 (s, 1H), 7.72 (d, J=4.8 Hz, 1H), 7.57 (s, 1H), 7.09 (d, J=4.8 Hz, 1H), 7.03 (d, J=10.0 Hz, 2H), 5.72 (s, 1H), 4.47-4.39 (m, 1H), 4.20 (dd, J=12.6, 5.0 Hz, 1H), 2.85-2.73 (m, 3H), 2.62-2.52 (m, 7H), 2.48-2.36 (m, 5H), 2.21-2.08 (m, 3H), 2.02-1.90 (m, 5H), 1.63 (s, 6H), 1.52-1.43 (m, 2H). [M+H]⁺=778.6

### Example 193: 3-cyano-N-(2-((1R,4r)-4-(4-((4-((R)-2,6-dioxopiperidin-3-yl)-3,5-difluorophenyl)glycyl)piperazin-1-yl)cyclohexyl)-6-(2-hydroxypropan-2-yl)-2H-indazol-5-yl)pyrrolo[1,2-b]pyridazine-7-carboxamide

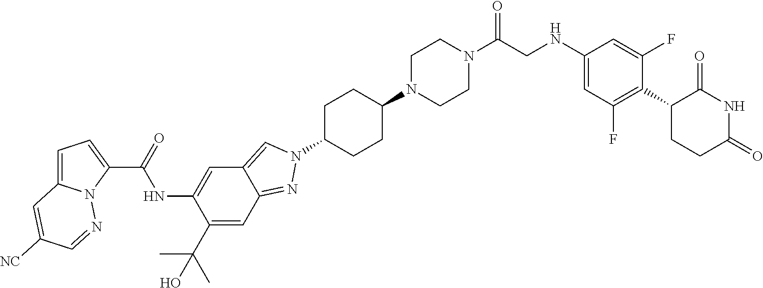 [SMILES: CC(C)(O)c1cc2nn([C@H]3CC[C@H](N4CCN(C(=O)CNc5cc(F)c([C@H]6CCC(=O)NC6=O)c(F)c5)CC4)CC3)cc2cc1NC(=O)c1ccc2cc(C#N)cnn12]

The title compound (18 mg, 28.9%) was prepared in a manner similar to that described in Example 178. ¹H NMR (500 MHz, DMSO) δ 12.02 (s, 1H), 10.83 (s, 1H), 8.93 (s, 1H), 8.74 (s, 1H), 8.55 (s, 1H), 8.34 (s, 1H), 7.72 (d, J=4.5 Hz, 1H), 7.57 (s, 1H), 7.09 (d, J=4.5 Hz, 1H), 6.41 (d, J=12.0 Hz, 2H), 6.23 (s, 1H), 5.72 (s, 1H), 4.48-4.38 (m, 1H), 4.03-3.97 (m, 1H), 3.95-3.86 (m, 2H), 3.55-3.40 (m, 5H), 2.83-2.72 (m, 1H), 2.62-2.54 (m, 3H), 2.22-2.14 (m, 2H), 2.10-2.03 (m, 1H), 2.00-1.88 (m, 6H), 1.63 (s, 6H), 1.56-1.40 (m, 3H). [M+H]⁺=807.6.

### Example 194: 3-cyano-N-(2-((1R,4r)-4-(4-(2-((4-((R)-2,6-dioxopiperidin-3-yl)-3,5-difluorophenyl)amino)ethyl)piperazin-1-yl)cyclohexyl)-6-(2-hydroxypropan-2-yl)-2H-indazol-5-yl)pyrrolo[1,2-b]pyridazine-7-carboxamide

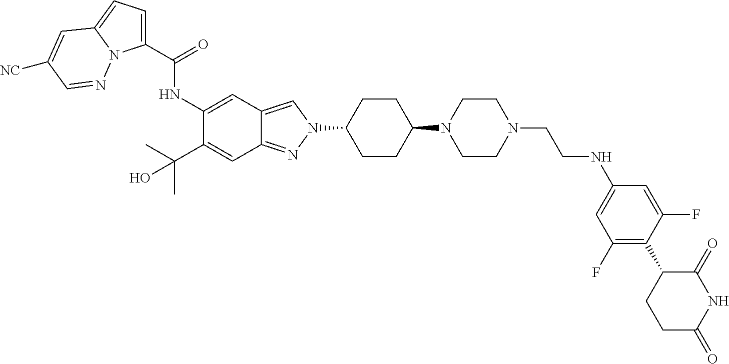 [SMILES: CC(C)(O)c1cc2nn([C@H]3CC[C@H](N4CCN(CCNc5cc(F)c([C@H]6CCC(=O)NC6=O)c(F)c5)CC4)CC3)cc2cc1NC(=O)c1ccc2cc(C#N)cnn12]

The title compound (52 mg, 57.9%) was prepared in a manner similar to that described in Example 9. ¹H NMR (500 MHz, DMSO) δ 12.02 (s, 1H), 10.83 (s, 1H), 8.93 (d, J=2.0 Hz, 1H), 8.74 (d, J=2.0 Hz, 1H), 8.55 (s, 1H), 8.33 (s, 1H), 7.72 (d, J=4.5 Hz, 1H), 7.57 (s, 1H), 7.09 (d, J=4.5 Hz, 1H), 6.27 (d, J=12.0 Hz, 2H), 6.07 (s, 1H), 5.72 (s, 1H), 4.48-4.38 (m, 1H), 3.98 (dd, J=12.5, 5.0 Hz, 1H), 3.14-3.11 (m, 3H), 2.80-2.74 (m, 1H), 2.70-2.55 (m, 6H), 2.40-2.27 (m, 3H), 2.21-2.16 (m, 2H), 2.10-1.92 (m, 7H), 1.63 (s, 6H), 1.56-1.48 (m, 2H), 1.22 (d, J=6.5 Hz, 1H). [M+H]⁺=793.5.

### Example 195: 3-cyano-N-(2-((1r,4r)-4-(4-(1-(4-(2,6-dioxopiperidin-3-yl)phenyl)piperidin-4-yl)piperazin-1-yl)cyclohexyl)-6-(2-hydroxypropan-2-yl)-2H-indazol-5-yl)pyrrolo[1,2-b]pyridazine-7-carboxamide

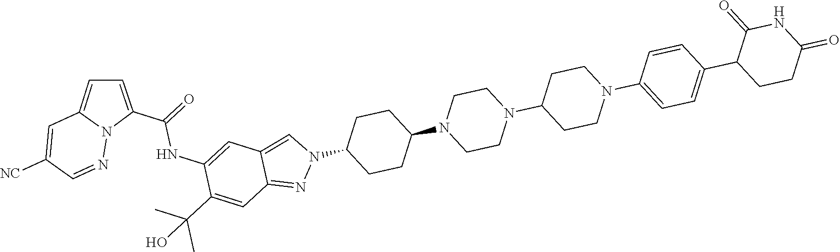 [SMILES: CC(C)(O)c1cc2nn([C@H]3CC[C@H](N4CCN(C5CCN(c6ccc(C7CCC(=O)NC7=O)cc6)CC5)CC4)CC3)cc2cc1NC(=O)c1ccc2cc(C#N)cnn12]

The title compound was prepared in a manner similar to that described in Example 9.

[M+H]⁺=797.5.

### Example 196: 3-cyano-N-(2-((1r,4r)-4-(4-(1-(4-((R)-2,6-dioxopiperidin-3-yl)-3,5-difluorophenyl)piperidin-4-yl)piperazin-1-yl)cyclohexyl)-6-(2-hydroxypropan-2-yl)-2H-indazol-5-yl)pyrrolo[1,2-b]pyridazine-7-carboxamide

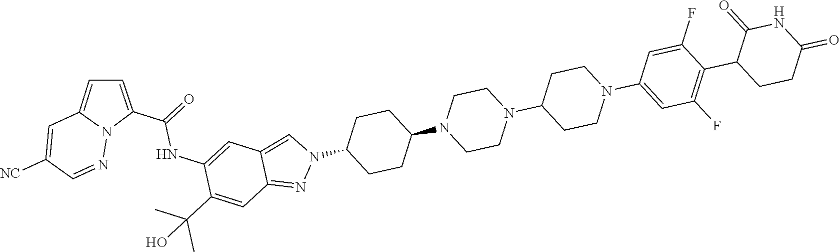 [SMILES: CC(C)(O)c1cc2nn([C@H]3CC[C@H](N4CCN(C5CCN(c6cc(F)c(C7CCC(=O)NC7=O)c(F)c6)CC5)CC4)CC3)cc2cc1NC(=O)c1ccc2cc(C#N)cnn12]

The title compound (6 mg, 9.4%) was prepared in a manner similar to that described in Example 9. ¹H NMR (500 MHz, DMSO) δ 12.02 (s, 1H), 10.87 (s, 1H), 8.93 (d, J=2.0 Hz, 1H), 8.74 (d, J=2.0 Hz, 1H), 8.55 (s, 1H), 8.33 (s, 1H), 7.72 (d, J=4.5 Hz, 1H), 7.57 (s, 1H), 7.09 (d, J=4.5 Hz, 1H), 6.62 (d, J=12.9 Hz, 2H), 5.72 (s, 1H), 4.45-4.38 (m, 1H), 4.08-4.03 (m, 1H), 3.78 (d, J=12.0 Hz, 2H), 3.56-3.47 (m, 2H), 2.80-2.70 (m, 4H), 2.58-2.52 (m, 5H), 2.40-2.30 (m, 3H), 2.18-2.07 (m, 3H), 1.98-1.90 (m, 5H), 1.82 (d, J=11.0 Hz, 2H), 1.63 (s, 6H), 1.49-1.39 (m, 4H). [M+H]⁺=833.6.

### Example 128: (S)—N-(2-(1′-(1-(4-(2,4-dioxotetrahydropyrimidin-1(2H)-yl)phenyl)pyrrolidine-3-carbonyl)-[1,4′-bipiperidin]-4-yl)-7-(2-hydroxypropan-2-yl)imidazo[1,2-a]pyridin-6-yl)-6-(trifluoromethyl)picolinamide

### Step 1: methyl 2-(1-(tert-butoxycarbonyl)piperidin-4-yl)-6-nitroimidazo[1,2-a]pyridine-7-carboxylate

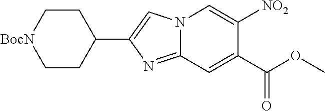 [SMILES: COC(=O)c1cc2nc(C3CCN(C(=O)OC(C)(C)C)CC3)cn2cc1[N+](=O)[O-]]

A mixture of methyl 2-amino-5-nitroisonicotinate (3.5 g, 17.77 mmol), tert-butyl 4-(2-bromoacetyl)piperidine-1-carboxylate (10.8 g, 35.54 mmol) and MgO (1.42 g, 35.54 mmol) in tetrahydrofuran (120 mL) was stirred in a sealing tube at 100° C. for 16 h. The mixture was evaporated under vacuum to afford the crude product, which was further purified with silica gel column chromatography (PE:EA=3:1˜1:2 gradient elution) to afford the product (3.9 g, 54.3%). [M+H]⁺=405.5.

### Step 2: methyl 6-amino-2-(1-(tert-butoxycarbonyl)piperidin-4-yl)imidazo[1,2-a]pyridine-7-carboxylate

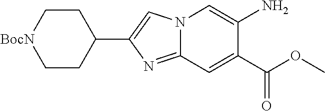 [SMILES: COC(=O)c1cc2nc(C3CCN(C(=O)OC(C)(C)C)CC3)cn2cc1N]

A solution of methyl 2-(1-(tert-butoxycarbonyl)piperidin-4-yl)-6-nitroimidazo[1,2-a]pyridine-7-carboxylate (3.9 g, 11.6 mmol), Fe (6.5 g, 116 mmol) and NH₄Cl (1.86 g, 34.8 mmol) in EtOH (80.0 mL) and water (20.0 mL) was stirred at 90° C. for 16 h. Reaction was monitored with LCMS. The mixture was filtered through a pad of Celite and washed with EtOH (60.0 mL). The filtrate was concentrated under vacuum and extracted with EA (3×15.0 mL) and washed with aqueous solution NaHCO₃ (20 mL). The organic layer was dried over anhydrous sodium sulfate and concentrated under vacuum to afford the product (2.5 g, 58%). [M+H]⁺=375.3.

### Step 3: methyl 2-(1-(tert-butoxycarbonyl)piperidin-4-yl)-6-(6-(trifluoromethyl)picolinamido)imidazo[1,2-a]pyridine-7-carboxylate

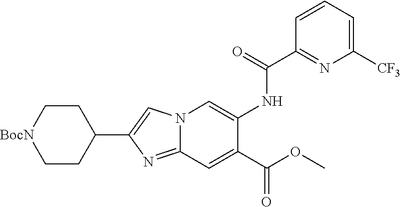 [SMILES: COC(=O)c1cc2nc(C3CCN(C(=O)OC(C)(C)C)CC3)cn2cc1NC(=O)c1cccc(C(F)(F)F)n1]

A mixture of 6-(trifluoromethyl)picolinic acid (1.28 g, 6.68 mmol), HATU (3.81 g, 10.03 mmol) and DIEA (2.6 g, 20.06 mmol) in DMF (100 mL) was stirred at room temperature for 30 minute, and methyl 6-amino-2-(1-(tert-butoxycarbonyl)piperidin-4-yl)imidazo[1,2-a]pyridine-7-carboxylate (2.5 g, 6.68 mmol) was added. The mixture was stirred at room temperature for 16 h. Reaction was monitored with LCMS. Water (80 mL) was added and the solid was filtrated, washed with water (50.0 mL), and dried to obtain the crude product, which was further purified with silica gel column chromatography (PE:EA=3:1˜1:2 gradient elution) to afford the product (2.5 g, 68.5%). [M+H]⁺=548.4.

### Step 4: tert-butyl 4-(7-(2-hydroxypropan-2-yl)-6-(6-(trifluoromethyl)picolinamido)imidazo[1,2-a]pyridin-2-yl)piperidine-1-carboxylate

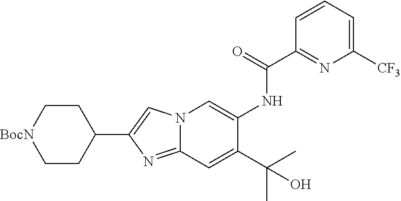 [SMILES: CC(C)(C)OC(=O)N1CCC(c2cn3cc(NC(=O)c4cccc(C(F)(F)F)n4)c(C(C)(C)O)cc3n2)CC1]

A mixture of MeMgCl (13.7 mL, 41.13 mmol, 3.0M) and LiCl (575.8 mg, 13.7 mmol) in THF (60 mL) was stirred at room temperature for 1 h. Methyl 2-(1-(tert-butoxycarbonyl)piperidin-4-yl)-6-(6-(trifluoromethyl)picolinamido)imidazo[1,2-a]pyridine-7-carboxylate (2.5 g, 4.57 mmol) in THF (20 mL) was added. The mixture was stirred at room temperature for 3 h. Reaction was monitored with LCMS. The reaction was quenched with aqueous solution NH₄Cl and extracted with DCM (2×50.0 mL) and water (20 mL). The organic layer was dried over anhydrous sodium sulfate and concentrated under vacuum to yield the crude product, which was further purified with silica gel column chromatography (PE:EA=3:1˜1:2 gradient elution) to afford the product (2.5 g, 100%). [M+H]⁺=548.4.

### Step 5: N-(7-(2-hydroxypropan-2-yl)-2-(piperidin-4-yl)imidazo[1,2-a]pyridin-6-yl)-6-(trifluoromethyl)picolinamide hydrochloride

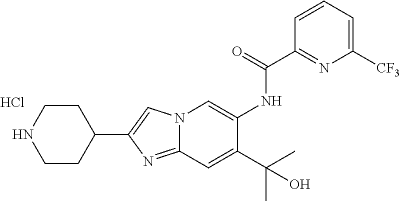 [SMILES: Cl ; CC(C)(O)c1cc2nc(C3CCNCC3)cn2cc1NC(=O)c1cccc(C(F)(F)F)n1]

Tert-butyl 4-(7-(2-hydroxypropan-2-yl)-6-(6-(trifluoromethyl)picolinamido)imidazo[1,2-a]pyridin-2-yl)piperidine-1-carboxylate (2 g, 3.65 mmol) in DCM (15 mL) was added 4N HCl/dioxane (40 mL) at 25° C. The mixture was stirred at 25° C. for 2 h. Reaction was monitored with LCMS. The solid was filtrated, washed with DCM (20.0 mL), and dried to obtain the product (1.5 g, 92%). [M+H]⁺=448.3.

### Step 6: tert-butyl 4-(7-(2-hydroxypropan-2-yl)-6-(6-(trifluoromethyl)picolinamido)imidazo[1,2-a]pyridin-2-yl)-[1,4′-bipiperidine]-1′-carboxylate

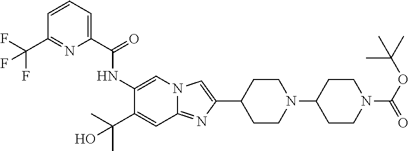 [SMILES: CC(C)(C)OC(=O)N1CCC(N2CCC(c3cn4cc(NC(=O)c5cccc(C(F)(F)F)n5)c(C(C)(C)O)cc4n3)CC2)CC1]

A mixture of N-(7-(2-hydroxypropan-2-yl)-2-(piperidin-4-yl)imidazo[1,2-a]pyridin-6-yl)-6-(trifluoromethyl)picolinamide hydrochloride (400 mg, 0.83 mmol) and tert-butyl 4-oxopiperidine-1-carboxylate (495 mg, 2.49 mmol) in DCM (30 mL) was stirred in a round bottom flask for 16 h at room temperature. Then NaBH(AcO)₃ (880 mg, 4.15 mmol) was added and stirred for 16 h at room temperature. The reaction was quenched with water and the mixture was washed once with saturated aqueous NaHCO₃, then extracted with DCM. The organic layer was dried over anhydrous Na₂SO₄, and evaporated under vacuum to afford the crude product (400 mg). [M+H]⁺=631.5.

### Step 7: N-(2-([1,4′-bipiperidin]-4-yl)-7-(2-hydroxypropan-2-yl)imidazo[1,2-a]pyridin-6-yl)-6-(trifluoromethyl)picolinamide hydrochloride

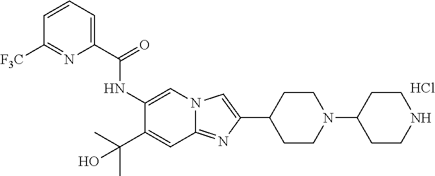 [SMILES: CC(C)(O)c1cc2nc(C3CCN(C4CCNCC4)CC3)cn2cc1NC(=O)c1cccc(C(F)(F)F)n1 ; Cl]

Tert-butyl 4-(7-(2-hydroxypropan-2-yl)-6-(6-(trifluoromethyl)picolinamido)imidazo[1,2-a]pyridin-2-yl)-[1,4′-bipiperidine]-1′-carboxylate (400 mg, 0.635 mmol) in DCM (25 mL) was added 4N HCl/dioxane (20 mL) at 25° C. The mixture was stirred at 25° C. for 2 h. Reaction was monitored with LCMS. The solid was filtrated, washed with DCM (20.0 mL), and dried to obtain the product (300 mg). [M+H]⁺=531.4.

### Step 8: (S)—N-(2-(1′-(1-(4-(2,4-dioxotetrahydropyrimidin-1(2H)-yl)phenyl)pyrrolidine-3-carbonyl)-[1,4′-bipiperidin]-4-yl)-7-(2-hydroxypropan-2-yl)imidazo[1,2-a]pyridin-6-yl)-6-(trifluoro methylpicolinamide

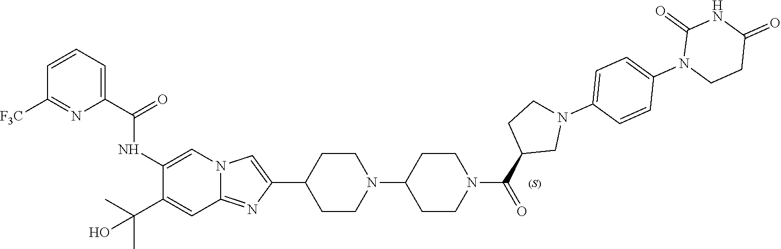

A mixture of (S)-1-(4-(2,4-dioxotetrahydropyrimidin-1(2H)-yl)phenyl)pyrrolidine-3-carboxylic acid (19 mg, 0.064 mmol), HATU (30 mg, 0.08 mmol) and DIEA (21 mg, 0.16 mmol) in DMF (8 mL) was stirred at room temperature for 20 minute, and N-(2-([1,4′-bipiperidin]-4-yl)-7-(2-hydroxypropan-2-yl)imidazo[1,2-a]pyridin-6-yl)-6-(trifluoromethyl)picolinamide hydrochloride (30 mg, 0.053 mmol) was added. The mixture was stirred at room temperature for 16 h. Reaction was monitored with LCMS. The resulting solution was extracted with DCM (2×15.0 mL) and washed with water (20 mL). The organic layer was dried over anhydrous sodium sulfate and concentrated under vacuum to yield the crude product. The crude product was purified with Prep-HPLC to afford the product (6.5 mg, 15%). ¹H NMR (500 MHz, DMSO) δ 12.47 (s, 1H), 10.22 (s, 1H), 9.31 (s, 1H), 8.49 (d, J=7.7 Hz, 1H), 8.42 (t, J=7.8 Hz, 1H), 8.24 (d, J=7.7 Hz, 1H), 7.67 (s, 1H), 7.11 (d, J=8.1 Hz, 2H), 6.55 (d, J=8.6 Hz, 3H), 4.60 (d, J=11.0 Hz, 1H), 4.25 (d, J=11.1 Hz, 1H), 3.73-3.52 (m, 7H), 3.32-3.07 (m, 5H), 2.68 (t, J=6.6 Hz, 2H), 2.65-2.54 (m, 2H), 2.38-2.28 (m, 2H), 2.25-2.04 (m, 5H), 1.98-1.85 (m, 2H), 1.66 (s, 7H), 1.60-1.49 (m, 1H). [M+H]⁺=816.5.

### Example 127: N-(2-(1′-((R)-1-(4-((R)-2,6-dioxopiperidin-3-yl)-3,5-difluorophenyl)pyrrolidine-3-carbonyl)-[1,4′-bipiperidin]-4-yl)-7-(2-hydroxypropan-2-yl)imidazo[1,2-a]pyridin-6-yl)-6-(trifluoromethyl)picolinamide

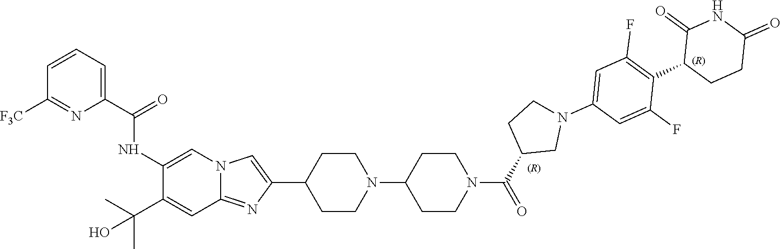

The title compound was prepared in a manner similar to that described in Example 128. ¹H NMR (500 MHz, DMSO) δ 12.41 (s, 1H), 10.85 (s, 1H), 9.80 (s, 1H), 9.36 (s, 1H), 8.48 (d, J=7.8 Hz, 1H), 8.42 (t, J=7.8 Hz, 1H), 8.23 (d, J=7.8 Hz, 1H), 7.42 (dd, J=17.2, 9.2 Hz, 1H), 6.43 (s, 1H), 6.22 (t, J=10.8 Hz, 2H), 4.59 (d, J=12.6 Hz, 1H), 4.22 (d, J=11.6 Hz, 1H), 4.05-3.99 (m, 1H), 3.65-3.51 (m, 5H), 3.25-3.02 (m, 7H), 2.84-2.72 (m, 1H), 2.67-2.57 (m, 1H), 2.39-2.26 (m, 2H), 2.25-2.02 (m, 6H), 1.99-1.85 (m, 3H), 1.75-1.59 (m, 7H), 1.58-1.48 (m, 1H). [M+H]⁺=851.5

### Example 126: (R)—N-(2-(1′-(1-(4-(2,6-dioxopiperidin-3-yl)-3,5-difluorophenyl)azetidine-3-carbonyl)-[1,4′-bipiperidin]-4-yl)-7-(2-hydroxypropan-2-yl)imidazo[1,2-a]pyridin-6-yl)-6-(trifluoromethyl)picolinamide

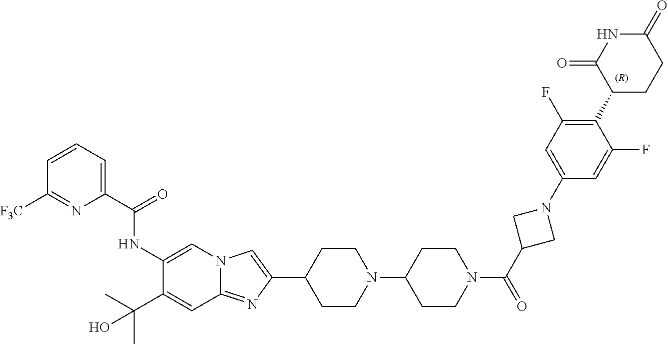

The title compound was prepared in a manner similar to that described in Example 128. ¹H NMR (500 MHz, DMSO) δ 12.38 (s, 1H), 10.79 (s, 1H), 9.78 (s, 1H), 9.50 (s, 1H), 8.41 (d, J=7.8 Hz, 1H), 8.36 (d, J=4.7 Hz, 1H), 8.17 (d, J=7.7 Hz, 1H), 7.63 (s, 1H), 6.43 (s, 1H), 6.10 (d, J=11.1 Hz, 2H), 4.50 (d, J=11.7 Hz, 1H), 4.06-3.93 (m, 3H), 3.93-3.76 (m, 3H), 3.71 (d, J=12.1 Hz, 1H), 3.19-3.07 (m, 3H), 3.06-2.95 (m, 2H), 2.92 (s, 2H), 2.77-2.66 (m, 1H), 2.56 (t, J=12.3 Hz, 1H), 2.31-2.20 (m, 2H), 2.09-1.94 (m, 4H), 1.94-1.81 (m, 3H), 1.64-1.53 (m, 7H), 1.53-1.43 (m, 1H). [M+H]⁺=837.5.

### Example 50: N-(2-(1-(1-((R)-1-(4-((R)-2,6-dioxopiperidin-3-yl)-3,5-difluorophenyl)pyrrolidine-3-carbonyl)azetidin-3-yl)piperidin-4-yl)-7-(2-hydroxypropan-2-yl)imidazo[1,2-a]pyridin-6-yl)-6-(trifluoromethyl)picolinamide

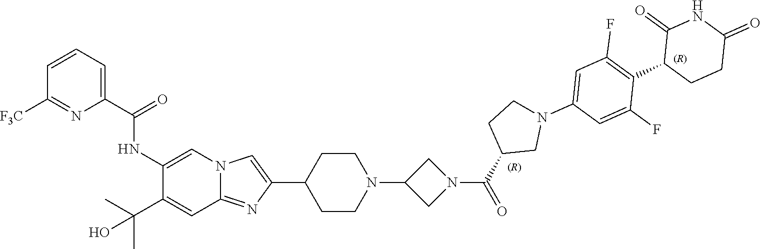

The title compound was prepared in a manner similar to that described in Example 128. ¹H NMR (500 MHz, DMSO) δ 12.43 (s, 1H), 10.85 (s, 1H), 9.82 (s, 1H), 8.48 (d, J=7.9 Hz, 1H), 8.42 (t, J=7.8 Hz, 1H), 8.23 (d, J=7.8 Hz, 2H), 7.66 (s, 1H), 6.47 (s, 1H), 6.23 (d, J=12.4 Hz, 2H), 4.59-4.41 (m, 2H), 4.20-4.07 (m, 3H), 4.02 (dd, J=12.3, 4.7 Hz, 2H), 3.92-2.89 (m, 7H), 2.83-2.72 (m, 2H), 2.35-2.25 (m, 2H), 2.23-2.13 (m, 2H), 2.14-2.02 (m, 3H), 1.98-1.91 (m, 1H), 1.91-1.80 (m, 2H), 1.65 (s, 6H). [M+H]⁺=823.5.

### Example 164: N-(2-((1R,4r)-4-(4-((R)-1-(4-(2,4-dioxotetrahydropyrimidin-1(2H)-yl)phenyl)pyrrolidine-3-carbonyl)piperazin-1-yl)cyclohexyl)-6-methoxy-2H-indazol-5-yl)-6-(trifluoromethyl)picolinamide

### Step 1: benzyl 4-(1,4-dioxaspiro[4.5]decan-8-yl)piperazine-1-carboxylate

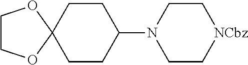 [SMILES: O=C(OCc1ccccc1)N1CCN(C2CCC3(CC2)OCCO3)CC1]

To a solution of 1,4-dioxaspiro[4.5]decan-8-one (50.0 g, 320 mmol) in DCM (1000 mL) was added benzyl piperazine-1-carboxylate (70.5 g, 320 mmol) at 0° C., then NaBH(OAc)₃ (101.8 g, 480 mmol) and AcOH (19.2 g, 320 mmol) were added. The mixture was stirred at 20° C. for 12 hours. The mixture was diluted with H₂O (500 mL) and extracted with DCM (500 mL×3). The combined organic layer was washed with 10 mL H₂O, 20 mL brine, dried over Na₂SO₄, filtered and concentrated to yield the crude product (51.0 g, 38% yield). ¹H NMR (400 MHz, CDCl₃-d) δ=7.45-7.29 (m, 5H), 5.12 (s, 2H), 3.97-3.89 (m, 5H), 3.66-3.59 (m, 4H), 2.81-2.65 (m, 5H), 1.90-1.77 (m, 5H), 1.69-1.52 (m, 5H).

### Step 2: benzyl 4-(4-oxocyclohexyl)piperazine-1-carboxylate

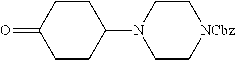 [SMILES: O=C1CCC(N2CCN(C(=O)OCc3ccccc3)CC2)CC1]

A mixture of benzyl 4-(1,4-dioxaspiro[4.5]decan-8-yl)piperazine-1-carboxylate (50.0 g, 139 mmol), HCl (6 N, 1430 mL) in dioxane (1000 mL) was evacuated and backfilled with N₂ for 3 times, and then the mixture was stirred at 20° C. for 12 hours under N₂ atmosphere. The aqueous phase was adjusted to pH 9 with solid NaHCO₃, the reaction mixture was quenched with H₂O (500 mL) at 20° C., and then extracted with EA (500 mL×3). The combined organic layer was washed with brine (500 mL×3), dried over Na₂SO₄, filtered and concentrated under reduced pressure to yield product (48.0 g, 97%). ¹HNMR (400 MHz, CDCl₃-d) δ=7.39-7.29 (m, 5H), 5.17-5.09 (m, 2H), 3.56-3.46 (m, 4H), 2.63-2.75 (m, 1H), 2.60-2.44 (m, 6H), 2.35-2.25 (m, 2H), 2.01-1.91 (m, 2H), 1.90-1.73 (m, 2H).

### Step 3: benzyl 4-((1s,4s)-4-hydroxycyclohexyl)piperazine-1-carboxylate

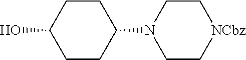 [SMILES: O=C(OCc1ccccc1)N1CCN([C@H]2CC[C@@H](O)CC2)CC1]

To a solution of benzyl 4-(4-oxocyclohexyl)piperazine-1-carboxylate (30.0 g, 95 mmol) in THF (300 mL) was added lithium trisec-butylboranuide THF solution (1 M, 190 mL) at 0° C. The mixture was stirred at 0° C. for 1 hour under N₂ atmosphere. The reaction mixture was quenched with H₂O (200 mL) at 20° C., and then extracted with EA (200 mL×3). The combined organic layer was washed with brine (100 mL×3), dried over Na₂SO₄, filtered and concentrated under reduced pressure to yield product (30.0 g, 99%). ¹H NMR (400 MHz, CDCl₃-d) δ=7.42-7.30 (m, 5H), 5.14 (s, 2H), 4.00 (t, J=3.0 Hz, 1H), 3.63 (s, 4H), 2.75-2.56 (m, 4H), 2.54-2.41 (m, 1H), 1.91-1.82 (m, 2H), 1.79-1.65 (m, 4H), 1.61-1.49 (m, 2H).

### Step 4: benzyl 4-((1s,4s)-4-((methylsulfonyl)oxy)cyclohexyl)piperazine-1-carboxylate

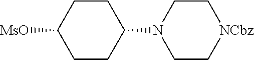 [SMILES: CS(=O)(=O)O[C@H]1CC[C@@H](N2CCN(C(=O)OCc3ccccc3)CC2)CC1]

To a solution of benzyl 4-((1s,4s)-4-hydroxycyclohexyl)piperazine-1-carboxylate (16 g, 50 mmol) in DCM (120 mL) was added TEA (20.34 g, 200.00 mmol) and MsCl (17.27 g, 150 mmol) at 0° C. The mixture was stirred at 20° C. for 6 hours. The reaction mixture was quenched with aq. NaHCO₃ (20 mL) at 20° C., and extracted with DCM (100 mL×3). The combined organic layer was washed with brine (100 mL×3), dried over Na₂SO₄, filtered and concentrated under reduced pressure to afford product (6.3 g, 28%). ¹HNMR (400 MHz, CDCl₃-d) δ=7.41-7.31 (m, 5H), 5.13 (s, 2H), 4.94 (s, 1H), 3.93 (dd, J=4.0, 7.2 Hz, 4H), 3.15-3.13 (m, 1H), 3.14-2.93 (m, 6H), 2.23-2.35 (m, 2H), 2.08-2.24 (m, 2H), 1.90-1.80 (m, 2H), 1.76-1.65 (m, 2H), 1.00-0.74 (m, 1H).

### Step 5: benzyl 4-((1r,4r)-4-(6-methoxy-5-(6-(trifluoromethyl)picolinamido)-2H-indazol-2-yl)cyclohexyl)piperazine-1-carboxylate

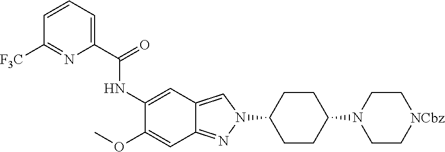 [SMILES: COc1cc2nn([C@H]3CC[C@@H](N4CCN(C(=O)OCc5ccccc5)CC4)CC3)cc2cc1NC(=O)c1cccc(C(F)(F)F)n1]

To a solution of benzyl 4-((1s,4s)-4-((methylsulfonyl)oxy)cyclohexyl)piperazine-1-carboxylate (4.0 g, 10.0 mmol) and N-(6-methoxy-2H-indazol-5-yl)-6-(trifluoromethyl)pyridine-2-carboxamide (754 mg, 2.2 mmol) in DMF (80 mL) was added Cs₂CO₃ (3.7 g, 11.2 mmol) at 20° C. The mixture was stirred at 80° C. for 12 hours. The reaction mixture was quenched with H₂O (20 mL) at 20° C., and extracted with EA (10 mL×3). The combined organic layer was washed with brine (10 mL×3), dried over Na₂SO₄, filtered and concentrated under reduced pressure to afford a residue. The residue was purified with prep-HPLC to get product (70 mg, 5%). ¹H NMR (400 MHz, CDCl₃-d) δ=10.71 (s, 1H), 8.87-8.79 (m, 1H), 8.50 (d, J=7.8 Hz, 1H), 8.12 (t, J=7.8 Hz, 1H), 7.92-7.80 (m, 2H), 7.41-7.31 (m, 5H), 7.07 (s, 1H), 5.15 (s, 2H), 4.36-4.27 (m, 1H), 4.07-4.05 (m, 1H), 4.03 (s, 2H), 3.56 (br s, 4H), 2.61 (br s, 4H), 2.37 (br d, J=12.0 Hz, 2H), 2.10 (br s, 2H), 2.02 (br d, J=12.4 Hz, 2H), 1.59-1.51 (m, 2H), 0.93-0.82 (m, 1H).

### Step 6: N-(6-methoxy-2-((1r,4r)-4-(piperazin-1-yl)cyclohexyl)-2H-indazol-5-yl)-6-(trifluoromethyl)picolinamide

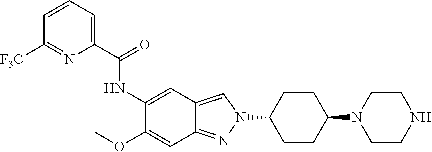 [SMILES: COc1cc2nn([C@H]3CC[C@H](N4CCNCC4)CC3)cc2cc1NC(=O)c1cccc(C(F)(F)F)n1]

To a solution of benzyl 4-((1r,4r)-4-(6-methoxy-5-(6-(trifluoromethyl)picolinamido)-2H-indazol-2-yl)cyclohexyl)piperazine-1-carboxylate (70.0 mg, 0.11 mmol) in EtOH (5 mL) and methylamine (1 mL) was added Pd(OH)₂ (15 mg, 0.11 mmol) and purged with H₂. The mixture was stirred at 20° C. for 12 hours under H₂. The suspension was filtered and the filter cake was washed with EtOH (10 mL×3). The combined filtrate was concentrated to dryness to afford product (20 mg, 33%). ¹H NMR (400 MHz, DMSO-d6) δ=10.50 (s, 1H), 8.68 (s, 1H), 8.43 (d, J=19.6 Hz, 2H), 8.32 (s, 1H), 8.21 (d, J=7.6 Hz, 1H), 7.15 (s, 1H), 4.35 (br s, 2H), 3.99-3.95 (m, 3H), 2.71-2.65 (m, 4H), 2.46-2.40 (m, 4H), 2.25-2.09 (m, 2H), 1.92 (br t, J=11.2 Hz, 4H), 1.53-1.41 (m, 4H).

### Step 7: N-(2-((1R,4r)-4-(4-((R)-1-(4-(2,4-dioxotetrahydropyrimidin-1(2H)-yl)phenyl)pyrrolidine-3-carbonyl)piperazin-1-yl)cyclohexyl)-6-methoxy-2H-indazol-5-yl)-6-(trifluoromethyl)picolinamide

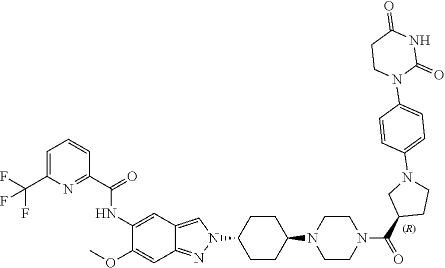

To a solution of N-(6-methoxy-2-((1r,4r)-4-(piperazin-1-yl)cyclohexyl)-2H-indazol-5-yl)-6-(trifluoromethyl)picolinamide (100 mg, 0.2 mmol), (R)-1-(4-(2,4-dioxotetrahydropyrimidin-1(2H)-yl)phenyl)pyrrolidine-3-carboxylic acid (60 mg, 0.2 mmol) and DIEA (129 mg, 1.0 mmol) in DCM (10 mL) was added 50% T3P in EA (159 mg, 0.2 mmol) at room temperature. The resulting mixture was stirred at room temperature for 18 hours. The reaction was evaporated under vacuum to afford the crude residue, which was purified with Prep-HPLC to afford product (55 mg, 35%). ¹H NMR (500 MHz, DMSO) δ 10.50 (s, 1H), 10.21 (s, 1H), 8.69 (s, 1H), 8.52-8.44 (m, 1H), 8.41 (t, J=7.5 Hz, 1H), 8.33 (s, 1H), 8.22 (d, J=8.0 Hz, 1H), 7.16 (s, 1H), 7.10 (d, J=8.5 Hz, 2H), 6.54 (d, J=8.5 Hz, 2H), 4.45-4.33 (m, 1H), 3.98 (s, 3H), 3.67 (t, J=6.5 Hz, 2H), 3.61-3.42 (m, 6H), 3.37-3.33 (m, 1H), 3.30-3.24 (m, 4H), 2.72-2.65 (m, 2H), 2.62-2.53 (m, 3H), 2.23-2.15 (m, 3H), 2.13-2.06 (m, 1H), 2.01-1.87 (m, 4H), 1.57-1.45 (m, 2H). [M+H]⁺=788.70.

### Example 37: N-(2-((1r,4r)-4-(4-(1-(4-(2,4-dioxotetrahydropyrimidin-1(2H)-yl)phenyl)azetidine-3-carbonyl)piperazin-1-yl)cyclohexyl)-6-methoxy-2H-indazol-5-yl)-6-(trifluoromethyl)picolinamide

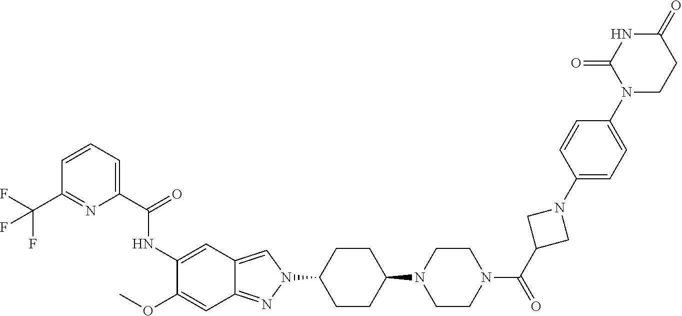 [SMILES: COc1cc2nn([C@H]3CC[C@H](N4CCN(C(=O)C5CN(c6ccc(N7CCC(=O)NC7=O)cc6)C5)CC4)CC3)cc2cc1NC(=O)c1cccc(C(F)(F)F)n1]

To a solution of N-(6-methoxy-2-((1r,4r)-4-(piperazin-1-yl)cyclohexyl)-2H-indazol-5-yl)-6-(trifluoromethyl)picolinamide (100 mg, 0.2 mmol), 1-(4-(2,4-dioxotetrahydropyrimidin-1(2H)-yl)phenyl)azetidine-3-carboxylic acid (58 mg, 0.2 mmol) and DIEA (258 mg, 2.0 mmol) in DCM (10 mL) was added 50% T3P in EA (318 mg, 0.5 mmol) at room temperature. The resulting mixture was stirred at room temperature for 18 hours, with about 40% starting material remaining. The reaction mixture was added HATU (40 mg, 0.1 mmol) and stirred at room temperature for extra 18 hours. After evaporated under vacuum to afford the crude residue, which was purified with Prep-HPLC to afford product (48 mg, 31%). ¹H NMR (500 MHz, DMSO) δ 10.50 (s, 1H), 10.24 (s, 1H), 8.69 (s, 1H), 8.46 (d, J=7.5 Hz, 1H), 8.41 (t, J=8.0 Hz, 1H), 8.33 (s, 1H), 8.22 (d, J=7.5 Hz, 1H), 8.13 (s, 1H), 7.15 (s, 1H), 7.12 (d, J=8.5 Hz, 2H), 6.45 (d, J=8.5 Hz, 2H), 4.46-4.33 (m, 1H), 4.03 (t, J=7.0 Hz, 2H), 3.92-3.80 (m, 3H), 3.67 (t, J=6.5 Hz, 2H), 3.57-3.22 (m, 4H), 2.67 (t, J=6.5 Hz, 2H), 2.61-2.52 (m, 3H), 2.48-2.40 (m, 2H), 2.28-2.12 (m, 2H), 2.06-1.83 (m, 4H), 1.66-1.39 (m, 3H), 0.98-0.88 (m, 1H). [M+H]⁺=774.50.

### Example 98: N-(2-((1r,4r)-4-(4-(1-(4-((R)-2,6-dioxopiperidin-3-yl)-3,5-difluorophenyl)azetidine-3-carbonyl)piperazin-1-yl)cyclohexyl)-6-(oxetan-3-yloxy)-2H-pyrazolo[3,4-b]pyridin-5-yl)-6-(trifluoromethyl)picolinamide

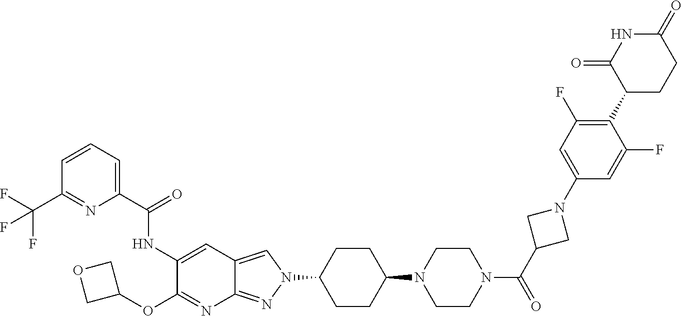 [SMILES: O=C1CC[C@H](c2c(F)cc(N3CC(C(=O)N4CCN([C@H]5CC[C@H](n6cc7cc(NC(=O)c8cccc(C(F)(F)F)n8)c(OC8COC8)nc7n6)CC5)CC4)C3)cc2F)C(=O)N1]

The title compound (10 mg, 31.7% yield) was prepared in a manner similar to that described in Example 164. ¹H NMR (500 MHz, DMSO) δ 10.86 (s, 1H), 10.54 (s, 1H), 8.95 (s, 1H), 8.48-8.43 (m, 2H), 8.36 (s, 1H), 8.26 (d, J=5.0 Hz, 1H), 6.17 (d, J=10.0 Hz, 2H), 5.76-5.74 (m, 1H), 5.05 (t, J=5.0 Hz, 2H), 4.67 (t, J=5.0 Hz, 2H), 4.38-4.34 (m, 1H), 4.05-4.02 (m, 2H), 3.92-3.83 (m, 3H), 3.48 (s, 2H), 2.81-2.75 (m, 2H), 2.57-2.48 (m, 6H), 2.16-2.07 (m, 5H), 1.95-1.88 (m, 5H), 1.49-1.47 (m, 2H). [M+H]⁺=852.6.

### Example 120: N-(2-((1R,4r)-4-(4-(4-((R)-2,6-dioxopiperidin-3-yl)-3,5-difluorophenethyl)piperazin-1-yl)cyclohexyl)-6-methoxy-2H-indazol-5-yl)-6-(trifluoromethyl)picolinamide

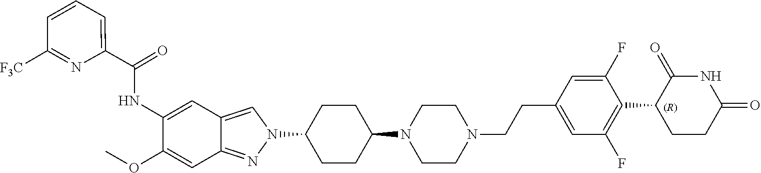

The title compound was prepared in a manner similar to that described in Example 9 with N-(6-methoxy-2-((1r,4r)-4-(piperazin-1-yl)cyclohexyl)-2H-indazol-5-yl)-6-(trifluoromethyl)picolinamide. ¹H NMR (500 MHz, DMSO) δ 10.95 (s, 1H), 10.50 (s, 1H), 8.68 (s, 1H), 8.56-8.35 (m, 2H), 8.32 (s, 1H), 8.22 (d, J=7.7 Hz, 1H), 7.16 (s, 1H), 7.03 (d, J=10.3 Hz, 2H), 4.41-4.35 (m, 1H), 4.20 (dd, J=15.5 Hz, 1H), 3.98 (s, 3H), 2.89-2.71 (m, 3H), 2.65-2.52 (m, 6H), 2.49-2.30 (m, 5H), 2.21-2.07 (m, 3H), 2.05-1.85 (m, 5H), 1.54-1.39 (m, 2H). [M+H]⁺=753.5

### Example 38: N-(2-((1r,4r)-4-(4-(1-(4-((R)-2,6-dioxopiperidin-3-yl)-3,5-difluorophenyl)azetidine-3-carbonyl)piperazin-1-yl)cyclohexyl)-6-(2-hydroxypropan-2-yl)-2H-indazol-5-yl)-6-(trifluoromethyl)picolinamide

### Step 1: benzyl 4-((1r,4r)-4-(6-(2-hydroxypropan-2-yl)-5-(6-(trifluoromethyl)picolinamido)-2H-indazol-2-yl)cyclohexyl)piperazine-1-carboxylate

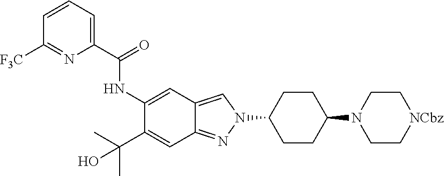 [SMILES: CC(C)(O)c1cc2nn([C@H]3CC[C@H](N4CCN(C(=O)OCc5ccccc5)CC4)CC3)cc2cc1NC(=O)c1cccc(C(F)(F)F)n1]

To a solution of N-[6-(1-hydroxy-1-methyl-ethyl)-2H-indazol-5-yl]-6-(trifluoromethyl)pyridine-2-carboxamide (obtained through the same way described in WO 2017186693 A1) (1.0 g, 2.7 mmol) and benzyl 4-(4-methylsulfonyloxycyclohexyl)piperazine-1-carboxylate (5.4 g, 13.7 mmol) in DMF (10 mL) was added Cs₂CO₃ (4.5 g, 13.7 mmol). The mixture was stirred at 80° C. for 12 hours. The mixture was cooled to rt, and diluted with H₂O (30 mL) and extracted with EA (50 mL×3). The combined organic layer was washed with 20 mL H₂O, 20 mL brine, dried over Na₂SO₄, filtered, and concentrated and purified with prep-HPLC to get product (300 mg, 16%). ¹H NMR (400 MHz, CDCl₃-d) δ ppm 12.28 (s, 1H), 8.87 (s, 1H), 8.51 (d, J=8.0 Hz, 1H), 8.12 (t, J=7.50 Hz, 1H), 7.91 (s, 1H), 7.85 (d, J=7.50 Hz, 1H), 7.74 (s, 1H), 7.33-7.40 (m, 5H), 5.15 (s, 2H), 4.36 (br t, J=11.60 Hz, 1H), 3.55 (s, 4H), 2.59 (s, 4H), 2.36 (d, J=13.20 Hz, 2H), 1.96-2.14 (m, 5H), 1.81 (s, 6H), 1.46-1.54 (m, 2H). [M+H]⁺=665.4.

### Step 2: N-(6-(2-hydroxypropan-2-yl)-2-((1r,4r)-4-(piperazin-1-yl)cyclohexyl)-2H-indazol-5-yl)-6-(trifluoromethyl)picolinamide

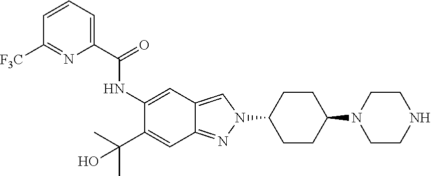 [SMILES: CC(C)(O)c1cc2nn([C@H]3CC[C@H](N4CCNCC4)CC3)cc2cc1NC(=O)c1cccc(C(F)(F)F)n1]

To a solution of benzyl 4-((1r,4r)-4-(6-(2-hydroxypropan-2-yl)-5-(6-(trifluoromethyl)picolinamido)-2H-indazol-2-yl)cyclohexyl)piperazine-1-carboxylate (150 mg, 0.2 mmol) in MeOH (10 mL) was added Pd/C (15 mg, 0.2 mmol, 10% wt) under N₂ atmosphere. The suspension was evacuated and backfilled with H₂ for 3 times. The mixture was stirred under H₂ (15 Psi) at 20° C. for 1 hour. The reaction mixture was filtered and concentrated to yield product (109 mg, 84% yield). ¹H NMR (400 MHz, CDCl₃-d) δ ppm 12.29 (s, 1H), 8.87 (s, 1H), 8.50 (d, J=7.60 Hz, 1H), 8.11 (t, J=7.60 Hz, 1H), 7.91 (s, 1H), 7.84 (d, J=7.70 Hz, 1H), 7.74 (s, 1H), 4.34 (d, J=8.80 Hz, 1H), 2.94 (d, J=10.80 Hz, 2H), 2.53-2.68 (m, 6H), 2.31 (br d, J=10.88 Hz, 3H), 1.94-2.19 (m, 4H), 1.80 (s, 6H), 1.46-1.58 (m, 2H). [M+H]⁺=531.2.

### Step 3: N-(2-((1r,4r)-4-(4-(1-(4-((R)-2,6-dioxopiperidin-3-yl)-3,5-difluorophenyl)azetidine-3-carbonyl)piperazin-1-yl)cyclohexyl)-6-(2-hydroxypropan-2-yl)-2H-indazol-5-yl)-6-(trifluoromethyl)picolinamide

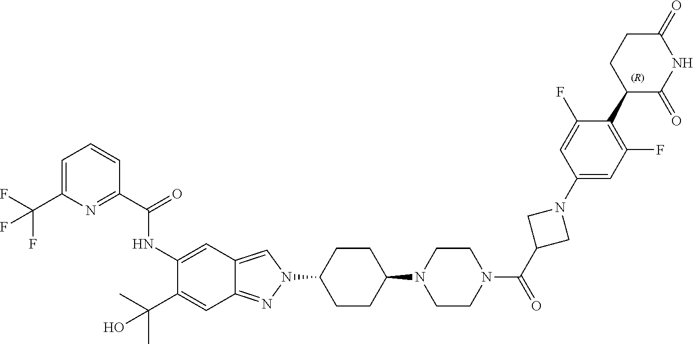

To a solution of N-(6-(2-hydroxypropan-2-yl)-2-((1r,4r)-4-(piperazin-1-yl)cyclohexyl)-2H-indazol-5-yl)-6-(trifluoromethyl)picolinamide (50 mg, 0.09 mmol), (R)-1-(4-(2,6-dioxopiperidin-3-yl)-3,5-difluorophenyl)azetidine-3-carboxylic acid (32 mg, 0.1 mmol) and DIPEA (65 mg, 0.5 mmol) in DMF (2.5 mL) was added HATU (38 mg, 0.1 mmol) at room temperature. The resulting mixture was stirred at room temperature for 18 hours. The reaction was evaporated under vacuum to afford the crude residue, which was purified with Prep-HPLC to afford product (37 mg, 44% yield). ¹H NMR (500 MHz, DMSO) δ 12.36 (s, 1H), 10.86 (s, 1H), 8.71 (s, 1H), 8.45 (d, J=7.5 Hz, 1H), 8.39-8.32 (m, 2H), 8.16 (d, J=7.5 Hz, 1H), 7.57 (s, 1H), 6.17 (d, J=11.0 Hz, 2H), 5.94 (s, 1H), 4.50-4.37 (m, 1H), 4.08-3.99 (m, 3H), 3.95-3.88 (m, 2H), 3.87-3.79 (m, 1H), 3.56-3.44 (m, 2H), 2.84-2.71 (m, 1H), 2.59-2.52 (m, 4H), 2.49-2.39 (m, 4H), 2.23-2.13 (m, 2H), 2.12-2.03 (m, 1H), 2.00-1.89 (m, 5H), 1.62 (s, 6H), 1.55-1.42 (m, 2H). [M+H]⁺=837.50.

### Example 39: N-(2-((1R,4r)-4-(4-((R)-1-(4-((R)-2,6-dioxopiperidin-3-yl)-3,5-difluorophenyl)pyrrolidine-3-carbonyl)piperazin-1-yl)cyclohexyl)-6-(2-hydroxypropan-2-yl)-2H-indazol-5-yl)-6-(trifluoromethyl)picolinamide

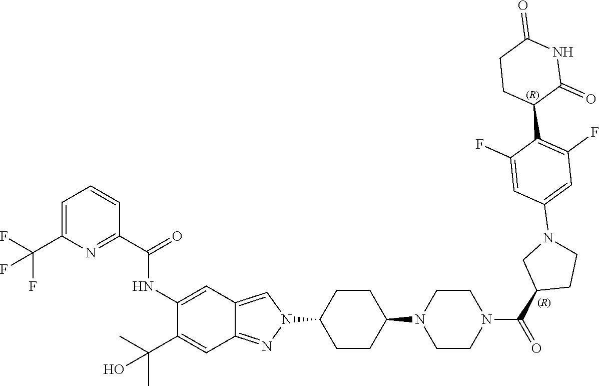

To a solution of N-(6-(2-hydroxypropan-2-yl)-2-((1r,4r)-4-(piperazin-1-yl)cyclohexyl)-2H-indazol-5-yl)-6-(trifluoromethyl)picolinamide (50 mg, 0.09 mmol), (R)-1-(4-((R)-2,6-dioxopiperidin-3-yl)-3,5-difluorophenyl)pyrrolidine-3-carboxylic acid (34 mg, 0.1 mmol) and DIPEA (65 mg, 0.5 mmol) in DMF (2.5 mL) was added HATU (38 mg, 0.1 mmol) at room temperature. The resulting mixture was stirred at room temperature for 18 hours. The reaction was evaporated under vacuum to afford the crude residue, which was purified with Prep-HPLC to afford product (37 mg, 44% yield). ¹H NMR (500 MHz, DMSO) δ 12.36 (s, 1H), 10.84 (s, 1H), 8.71 (s, 1H), 8.45 (d, J=7.5 Hz, 1H), 8.40-8.27 (m, 2H), 8.16 (d, J=8.0 Hz, 1H), 7.57 (s, 1H), 6.23 (d, J=12.0 Hz, 2H), 5.94 (s, 1H), 4.51-4.36 (m, 1H), 4.02 (dd, J=12.0, 4.5 Hz, 1H), 3.66-3.40 (m, 6H), 3.38-3.19 (m, 6H), 2.83-2.70 (m, 1H), 2.62-2.53 (m, 3H), 2.24-2.13 (m, 3H), 2.12-2.04 (m, 2H), 2.01-1.89 (m, 5H), 1.62 (s, 6H), 1.56-1.46 (m, 2H). [M+H]⁺=851.60.

### Example 40: N-(2-((1r,4r)-4-(4-(1-(4-(2,4-dioxotetrahydropyrimidin-1(2H)-yl)phenyl)azetidine-3-carbonyl)piperazin-1-yl)cyclohexyl)-6-(2-hydroxypropan-2-yl)-2H-indazol-5-yl)-6-(trifluoromethyl)picolinamide

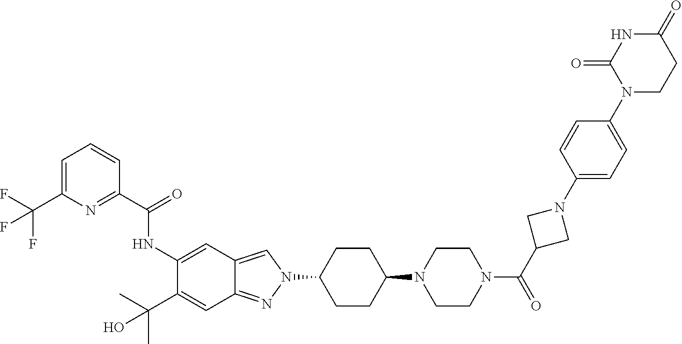 [SMILES: CC(C)(O)c1cc2nn([C@H]3CC[C@H](N4CCN(C(=O)C5CN(c6ccc(N7CCC(=O)NC7=O)cc6)C5)CC4)CC3)cc2cc1NC(=O)c1cccc(C(F)(F)F)n1]

To a solution of N-(6-(2-hydroxypropan-2-yl)-2-((1r,4r)-4-(piperazin-1-yl)cyclohexyl)-2H-indazol-5-yl)-6-(trifluoromethyl)picolinamide (50 mg, 0.09 mmol), 1-(4-(2,4-dioxotetrahydropyrimidin-1(2H)-yl)phenyl)azetidine-3-carboxylic acid (29 mg, 0.1 mmol) and DIPEA (65 mg, 0.5 mmol) in DMF (2.5 mL) was added HATU (38 mg, 0.1 mmol) at room temperature. The resulting mixture was stirred at room temperature for 18 hours. The reaction was evaporated under vacuum to afford the crude residue, which was purified with Prep-HPLC to yield product (37 mg, 44% yield). ¹H NMR (500 MHz, DMSO) δ 12.36 (s, 1H), 10.23 (s, 1H), 8.71 (s, 1H), 8.45 (d, J=7.5 Hz, 1H), 8.39-8.32 (m, 2H), 8.16 (d, J=7.5 Hz, 1H), 7.57 (s, 1H), 7.11 (d, J=8.5 Hz, 2H), 6.45 (d, J=8.5 Hz, 2H), 5.94 (s, 1H), 4.49-4.37 (m, 1H), 4.02 (t, J=7.0 Hz, 2H), 3.90-3.76 (m, 3H), 3.67 (t, J=6.5 Hz, 2H), 3.54-3.43 (m, 2H), 2.67 (t, J=6.5 Hz, 2H), 2.58-2.51 (m, 4H), 2.48-2.43 (m, 3H), 2.23-2.12 (m, 2H), 2.01-1.86 (m, 4H), 1.62 (s, 6H), 1.56-1.42 (m, 2H). [M+H]⁺=802.50.

### Example 47: N-(2-((1r,4r)-4-(4-(1-(4-((R)-2,6-dioxopiperidin-3-yl)-3,5-difluorophenyl)piperidine-4-carbonyl)piperazin-1-yl)cyclohexyl)-6-(2-hydroxypropan-2-yl)-2H-indazol-5-yl)-6-(trifluoromethyl)picolinamide

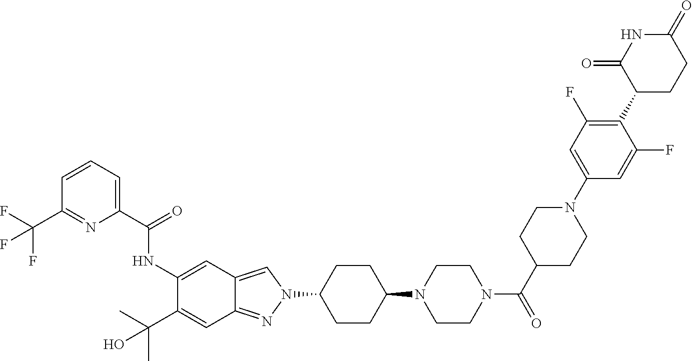 [SMILES: CC(C)(O)c1cc2nn([C@H]3CC[C@H](N4CCN(C(=O)C5CCN(c6cc(F)c([C@H]7CCC(=O)NC7=O)c(F)c6)CC5)CC4)CC3)cc2cc1NC(=O)c1cccc(C(F)(F)F)n1]

The title compound was prepare in a manner similar to a described in Example 38. ¹H NMR (500 MHz, DMSO) δ 12.36 (s, 1H), 10.87 (s, 1H), 8.71 (s, 1H), 8.46-8.36 (m, 3H), 8.16 (d, J=10.0 Hz, 1H), 7.57 (s, 1H), 7.63 (d, J=10.0 Hz, 2H), 5.94 (s, 1H), 4.44-4.42 (m, 1H), 4.06-4.04 (m, 1H), 3.80-3.77 (m, 2H), 3.54-3.46 (m, 4H), 2.85-2.76 (m, 5H), 2.63-2.53 (m, 5H), 2.19-2.05 (m, 3H), 1.96-1.94 (m, 5H), 1.65-1.49 (m, 12H). [M+H]⁺=865.7.

### Example 99: N-(2-((1r,4r)-4-(4-(1-(4-((R)-2,6-dioxopiperidin-3-yl)-3,5-difluorophenyl)piperidin-4-yl)piperazin-1-yl)cyclohexyl)-6-(2-hydroxypropan-2-yl)-2H-indazol-5-yl)-6-(trifluoromethyl)picolinamide

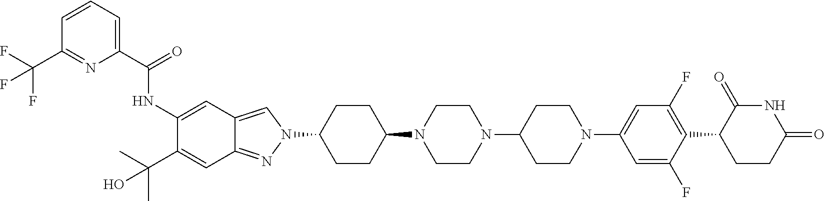 [SMILES: CC(C)(O)c1cc2nn([C@H]3CC[C@H](N4CCN(C5CCN(c6cc(F)c([C@H]7CCC(=O)NC7=O)c(F)c6)CC5)CC4)CC3)cc2cc1NC(=O)c1cccc(C(F)(F)F)n1]

The title compound (7.92 mg, 10.1% yield) was prepared with N-(6-(2-hydroxypropan-2-yl)-2-((1r,4r)-4-(piperazin-1-yl)cyclohexyl)-2H-indazol-5-yl)-6-(trifluoromethyl)picolinamide in a manner similar to that described in Example 9. ¹H NMR (500 MHz, DMSO) δ 12.36 (s, 1H), 10.87 (s, 1H), 8.71 (s, 1H), 8.46-8.35 (m, 3H), 8.16 (d, J=10.0 Hz, 1H), 7.57 (s, 1H), 6.64 (d, J=10.0 Hz, 2H), 5.94 (s, 1H), 4.42 (t, J=5.0 Hz, 1H), 4.05-4.03 (m, 1H), 3.79-3.77 (m, 2H), 2.82-2.71 (m, 5H), 2.39-2.34 (m, 4H), 2.17-2.48 (m, 6H), 2.16-2.05 (m, 6H), 1.97-1.90 (m, 6H), 1.83-1.81 (m, 2H), 1.62 (s, 6H), 1.47-1.42 (m, 5H). [M+H]⁺=837.6.

### Example 165: N-(2-((1r,4r)-4-(4-(1-(4-((R)-2,6-dioxopiperidin-3-yl)-3,5-difluorophenyl)azetidine-3-carbonyl)piperazin-1-yl)cyclohexyl)-6-(methoxy-d3)-2H-indazol-5-yl)-6-(trifluoromethyl)picolinamide

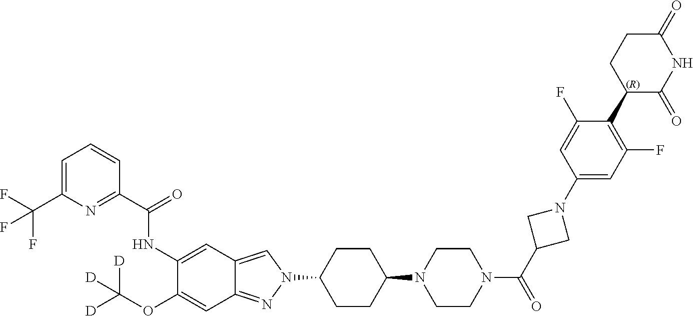

To a solution of N-(6-(methoxy-d3)-2-((1r,4r)-4-(piperazin-1-yl)cyclohexyl)-2H-indazol-5-yl)-6-(trifluoromethyl)picolinamide (obtained in a manner similar to Example 164) (50 mg, 0.1 mmol), (R)-1-(4-(2,6-dioxopiperidin-3-yl)-3,5-difluorophenyl)azetidine-3-carboxylic acid (32 mg, 0.1 mmol) and DIPEA (65 mg, 0.5 mmol) in DMF (2.5 mL) was added HATU (38 mg, 0.1 mmol) at room temperature. The resulting mixture was stirred at room temperature for 18 hours. The reaction was evaporated under vacuum to afford the crude residue, which was purified with Prep-HPLC to yield product (31 mg, 38%). ¹H NMR (500 MHz, DMSO) δ 10.86 (s, 1H), 10.50 (s, 1H), 8.68 (s, 1H), 8.46 (d, J=8.0 Hz, 1H), 8.41 (t, J=7.5 Hz, 1H), 8.33 (s, 1H), 8.21 (d, J=8.0 Hz, 1H), 7.15 (s, 1H), 6.17 (d, J=11.5 Hz, 2H), 4.45-4.32 (m, 1H), 4.04 (t, J=8.0 Hz, 3H), 3.92 (t, J=6.5 Hz, 2H), 3.87-3.78 (m, 1H), 3.55-3.43 (m, 2H), 3.30-3.28 (m, 1H), 2.84-2.73 (m, 1H), 2.56-2.51 (m, 4H), 2.48-2.43 (m, 3H), 2.22-2.13 (m, 2H), 2.13-2.02 (m, 1H), 1.99-1.86 (m, 5H), 1.56-1.41 (m, 2H). [M+H]⁺=812.50.

### Example 169: N-(2-((1R,4r)-4-(4-((R)-1-(4-((R)-2,6-dioxopiperidin-3-yl)-3,5-difluorophenyl)pyrrolidine-3-carbonyl)piperazin-1-yl)cyclohexyl)-6-(methoxy-d3)-2H-indazol-5-yl)-6-(trifluoromethyl)picolinamide

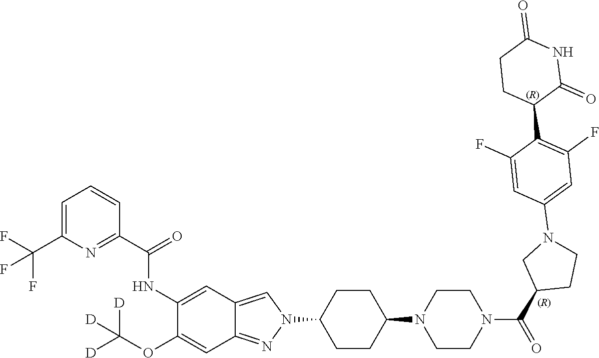

To a solution of N-(6-(methoxy-d3)-2-((1r,4r)-4-(piperazin-1-yl)cyclohexyl)-2H-indazol-5-yl)-6-(trifluoromethyl)picolinamide (50 mg, 0.09 mmol), (R)-1-(4-((R)-2,6-dioxopiperidin-3-yl)-3,5-difluorophenyl)pyrrolidine-3-carboxylic acid (34 mg, 0.1 mmol) and DIPEA (65 mg, 0.5 mmol) in DMF (2.5 mL) was added HATU (38 mg, 0.1 mmol) at room temperature. The resulting mixture was stirred at room temperature for 18 hours. The reaction was evaporated under vacuum to afford the crude residue, which was purified with Prep-HPLC to yield product (45 mg, 55%). ¹H NMR (500 MHz, DMSO) δ 10.85 (s, 1H), 10.51 (s, 1H), 8.69 (s, 1H), 8.46 (d, J=8.0 Hz, 1H), 8.41 (t, J=7.5 Hz, 1H), 8.33 (s, 1H), 8.22 (d, J=8.0 Hz, 1H), 7.15 (s, 1H), 6.23 (d, J=12.0 Hz, 2H), 4.46-4.36 (m, 1H), 4.02 (dd, J=12.5, 5.0 Hz, 1H), 3.64-3.50 (m, 3H), 3.49-3.42 (m, 2H), 3.37-3.33 (m, 2H), 3.31-3.23 (m, 3H), 2.82-2.74 (m, 1H), 2.54-2.51 (m, 3H), 2.50-2.47 (m, 2H), 2.27-2.15 (m, 3H), 2.13-2.02 (m, 3H), 2.00-1.86 (m, 4H), 1.73-1.38 (m, 2H). [M+H]⁺=826.60.

### Example 33: 1-cyclopropyl-N-(2-((1R,4r)-4-((4-((R)-1-(4-(2,4-dioxotetrahydropyrimidin-1(2H)-yl)phenyl)pyrrolidine-3-carbonyl)piperazin-1-yl)methyl)cyclohexyl)-6-methoxy-2H-indazol-5-yl)-2-oxo-1,2-dihydropyridine-3-carboxamide

### Step 1: 5-bromo-4-methoxy-2-nitrobenzaldehyde

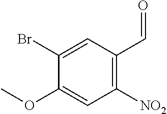

To a MeOH solution (200 mL) of 5-bromo-4-fluoro-2-nitro-benzaldehyde (20.0 g, 80.64 mmol) was added sodium methanolate (6.5 g, 120.96 mmol) at 0° C. The mixture was stirred at 20° C. under N₂ for 16 hours. The reaction mixture was poured into ice water, and the precipitate was filtered. The filter cake was washed with H₂O (100 mL), dried under to yield product (18.0 g, 80%). ¹H NMR (400 MHz, DMSO-d6) δ ppm 10.04 (s, 1H) 8.15 (s, 1H) 7.78 (s, 1H) 4.06 (s, 3H); [M+H]⁺=247.0.

### Step 2: ((1r,4r)-4-(5-bromo-6-methoxy-2H-indazol-2-yl)cyclohexyl)methanol

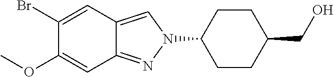 [SMILES: COc1cc2nn([C@H]3CC[C@H](CO)CC3)cc2cc1Br]

A mixture of 5-bromo-4-methoxy-2-nitrobenzaldehyde (18.0 g, 69.22 mmol) and ((1r,4r)-4-aminocyclohexyl)methanol (9.8 g, 76.14 mmol) in IPA (180 mL) was evacuated and backfilled with N₂ for 3 times, and the mixture was stirred at 80° C. under N₂ for 3 hours. Then the mixture was cooled to 20° C., tributylphosphane (42.0 g, 207.66 mmol, 51 mL) was added and the reaction mixture was stirred at 80° C. under N₂ for 16 hours. The mixture was diluted with H₂O (100 mL), extracted with EA (100 mL×3). The combined organic layer was washed with 50 mL H₂O, 50 mL brine, dried over Na₂SO₄, filtered and concentrated to afford crude product. The crude product was purified with column chromatography (SiO₂, Petroleum ether/Ethyl acetate=20/1 to 1/1) to afford the product (22.0 g, 93%). ¹H NMR (400 MHz, DMSO-d6) δ ppm 8.26 (s, 1H) 7.95 (s, 1H) 7.10 (s, 1H) 4.36 (ddt, J=11.51, 7.66, 3.86, 3.86 Hz, 1H) 3.86 (s, 3H) 3.28 (t, J=5.69 Hz, 2H) 2.07-2.14 (m, 2H) 1.86-1.92 (m, 3H) 1.56-1.61 (m, 2H) 1.07-1.21 (m, 2H); [M+H]⁺=339.1.

### Step 3: ((1r,4r)-4-(5-amino-6-methoxy-2H-indazol-2-yl)cyclohexyl)methanol

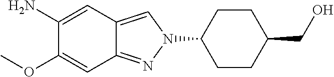 [SMILES: COc1cc2nn([C@H]3CC[C@H](CO)CC3)cc2cc1N]

A mixture of ((1r,4r)-4-(5-bromo-6-methoxy-2H-indazol-2-yl)cyclohexyl)methanol (4.0 g, 11.79 mmol), CuI (2.3 g, 11.79 mmol), L-PROLINE (1.6 g, 14.15 mmol) and NH₃·H₂O (21.8 g, 155.80 mmol, 24 mL, 25% purity) in DMSO (24 mL) was evacuated and backfilled with N₂ for 3 times, then the mixture was stirred at 80° C. under N₂ for 12 hours. Then the suspension was filtered and the filter cake was washed with EtOAc (500 mL×3). The combined filtrate was concentrated to dryness to yield crude product.

The crude product was purified with column chromatography (SiO₂, Petroleum ether/Ethyl acetate=10/1 to 0/1) to yield the product (10.0 g, 30%). ¹H NMR (400 MHz, DMSO-d6) δ ppm 7.85 (s, 1H) 6.83 (s, 1H) 6.61 (s, 1H) 4.56 (s, 2H) 4.47 (t, J=5.30 Hz, 1H) 4.17-4.27 (m, 1H) 3.81 (s, 3H) 3.27 (t, J=5.78 Hz, 2H) 2.04-2.11 (m, 2H) 1.77-1.92 (m, 4H) 1.44 (dtt, J=14.72, 5.89, 5.89, 3.08, 3.08 Hz, 1H) 1.05-1.17 (m, 2H); [M+H]⁺=276.2.

### Step 4: 1-cyclopropyl-N-(2-((1r,4r)-4-(hydroxymethyl)cyclohexyl)-6-methoxy-2H-indazol-5-yl)-2-oxo-1,2-dihydropyridine-3-carboxamide

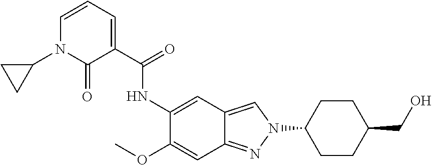 [SMILES: COc1cc2nn([C@H]3CC[C@H](CO)CC3)cc2cc1NC(=O)c1cccn(C2CC2)c1=O]

To a DMF solution (6 mL) of ((1r,4r)-4-(5-amino-6-methoxy-2H-indazol-2-yl)cyclohexyl)methanol (576 mg, 2.095 mmol) was added 1-cyclopropyl-2-oxo-1,2-dihydropyridine-3-carboxylic acid (450 mg, 2.514 mmol), HATU (1.2 g, 3.143 mmol) and DIEA (2 mL). The mixture was stirred overnight at room temperature under N₂. The solvent was removed under vaccum, and water (50 mL) was added and extracted with EA (50 mL×3). The organic layer was purified with combi flash, eluted with EA:PE=0:1˜1:0 to afford the product (860 mg, 94%). [M+H]⁺=437.2.

### Step 5: 1-cyclopropyl-N-(2-((1r,4r)-4-formylcyclohexyl)-6-methoxy-2H-indazol-5-yl)-2-oxo-1,2-dihydropyridine-3-carboxamide

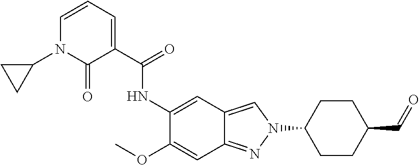 [SMILES: COc1cc2nn([C@H]3CC[C@H](C=O)CC3)cc2cc1NC(=O)c1cccn(C2CC2)c1=O]

To a DMSO solution (20 mL) of 1-cyclopropyl-N-(2-((1r,4r)-4-(hydroxymethyl)cyclohexyl)-6-methoxy-2H-indazol-5-yl)-2-oxo-1,2-dihydropyridine-3-carboxamide (860 mg, 1.9 mmol) was added IBX (1.06 g, 3.8 mmol). The mixture was stirred overnight at room temperature under N₂. To the mixture was added Na₂CO₃ aq. (50 mL) and extracted with DCM (50 mL×3). The organic layer was purified with combi flash, eluted with EA:PE=0:1˜1:0 to afford the product (700 mg, 85%). [M+H]⁺=435.2.

### Step 6: tert-butyl 4-(((1r,4r)-4-(5-(1-cyclopropyl-2-oxo-1,2-dihydropyridine-3-carboxamido)-6-methoxy-2H-indazol-2-yl)cyclohexyl)methyl)piperazine-1-carboxylate

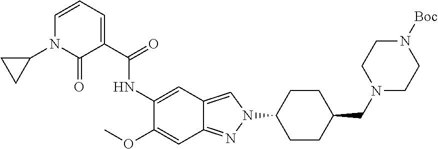 [SMILES: COc1cc2nn([C@H]3CC[C@H](CN4CCN(C(=O)OC(C)(C)C)CC4)CC3)cc2cc1NC(=O)c1cccn(C2CC2)c1=O]

To a DCM:MeOH (10 mL: 2 mL) solution of 1-cyclopropyl-N-(2-((1r,4r)-4-formylcyclohexyl)-6-methoxy-2H-indazol-5-yl)-2-oxo-1,2-dihydropyridine-3-carboxamide (700 mg, 1.61 mmol) was added tert-butyl piperazine-1-carboxylate (360 mg, 1.93 mmol) and AcOH (5 drops). The mixture was stirred at room temperature under N₂. To the mixture was added NaBH(Ac)₃ (119 mg, 0.56 mmol), then stirred for 0.5 h at room temperature under N₂. The reaction was quenched with Na₂CO₃ aq. (50 mL) and extracted with DCM (50 mL×3). The organic layer was dried over with Na₂SO₄, filtered and concentrated to afford the product (650 mg, 67%). [M+H]⁺=605.3.

### Step 7: 1-cyclopropyl-N-(6-methoxy-2-((1r,4r)-4-(piperazin-1-ylmethyl)cyclohexyl)-2H-indazol-5-yl)-2-oxo-1,2-dihydropyridine-3-carboxamide

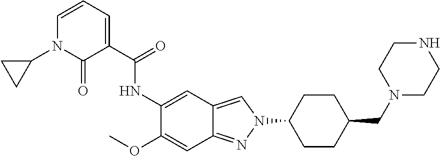 [SMILES: COc1cc2nn([C@H]3CC[C@H](CN4CCNCC4)CC3)cc2cc1NC(=O)c1cccn(C2CC2)c1=O]

To a DCM solution (10 mL) of tert-butyl 4-(((1r,4r)-4-(5-(1-cyclopropyl-2-oxo-1,2-dihydropyridine-3-carboxamido)-6-methoxy-2H-indazol-2-yl)cyclohexyl)methyl)piperazine-1-carboxylate (650 mg, 1.07 mmol) was added TFA (5 mL). The mixture was stirred overnight at room temperature under N₂. The solvent was removed under vacuum. Na₂CO₃ aq. (50 mL) was added to the residue and extracted with DCM (50 mL×3). The organic layer was dried over with Na₂SO₄, filtered and concentrated to afford the product (500 mg, 93%). [M+H]⁺=505.3.

### Step 8: 1-cyclopropyl-N-(2-((1R,4r)-4-((4-((R)-1-(4-(2,4-dioxotetrahydropyrimidin-1(2H)-yl)phenyl)pyrrolidine-3-carbonyl)piperazin-1-yl)methyl)cyclohexyl)-6-methoxy-2H-indazol-5-yl)-2-oxo-1,2-dihydropyridine-3-carboxamide

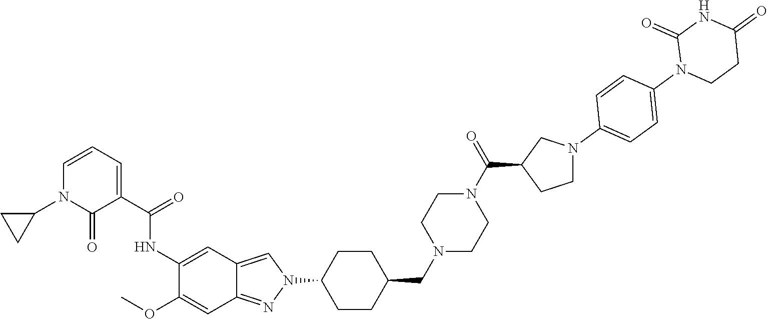 [SMILES: COc1cc2nn([C@H]3CC[C@H](CN4CCN(C(=O)[C@@H]5CCN(c6ccc(N7CCC(=O)NC7=O)cc6)C5)CC4)CC3)cc2cc1NC(=O)c1cccn(C2CC2)c1=O]

To a DCM solution (5 mL) of 1-cyclopropyl-N-(6-methoxy-2-((1r,4r)-4-(piperazin-1-ylmethyl)cyclohexyl)-2H-indazol-5-yl)-2-oxo-1,2-dihydropyridine-3-carboxamide (50 mg, 0.099 mmol) was added (R)-1-(4-(2,4-dioxotetrahydropyrimidin-1(2H)-yl)phenyl)pyrrolidine-3-carboxylic acid (36 mg, 0.1188 mmol), T₃P (6 drops) and DIEA (0.5 mL). The mixture was stirred overnight at room temperature under N₂. The result mixture was purified with combi flash, eluted with DCM:MeOH=1:0˜1:10 to yield the crude product which was further purified with prepHPLC to afford the product (15 mg, 19%). ¹H NMR (500 MHz, DMSO) δ 12.31 (s, 1H), 10.21 (s, 1H), 8.76 (s, 1H), 8.48 (d, J=5.0 Hz, 1H), 8.25 (s, 1H), 8.06 (d, J=5.0 Hz, 1H), 7.10 (d, J=10.0 Hz, 2H), 7.07 (s, 1H), 6.64-6.61 (m, 1H), 6.53 (d, J=10.0 Hz, 2H), 6.07-6.01 (m, 1H), 5.24 (d, J=10.0 Hz, 1H), 5.12 (d, J=10.0 Hz, 1H), 4.74 (d, J=5.0 Hz, 2H), 4.35 (t, J=10.0 Hz, 1H), 3.93 (s, 3H), 3.68-3.65 (m, 2H), 3.57-3.44 (m, 7H), 3.27-3.26 (m, 2H), 2.68 (t, J=10.0 Hz, 2H), 2.40-2.33 (m, 4H), 2.19-2.08 (m, 6H), 1.97-1.86 (m, 4H), 1.69-1.59 (m, 1H), 1.15-1.08 (m, 2H). [M+H]⁺=790.6.

### Example 34: 1-cyclopropyl-N-(2-((1S,4r)-4-((4-((S)-1-(4-(2,4-dioxotetrahydropyrimidin-1(2H)-yl)phenyl)pyrrolidine-3-carbonyl)piperazin-1-yl)methyl)cyclohexyl)-6-methoxy-2H-indazol-5-yl)-2-oxo-1,2-dihydropyridine-3-carboxamide

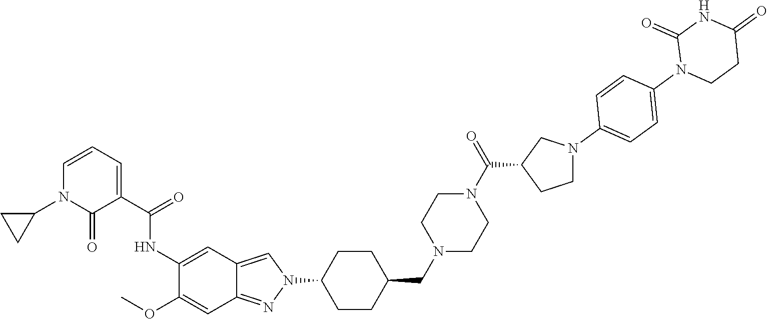 [SMILES: COc1cc2nn([C@H]3CC[C@H](CN4CCN(C(=O)[C@H]5CCN(c6ccc(N7CCC(=O)NC7=O)cc6)C5)CC4)CC3)cc2cc1NC(=O)c1cccn(C2CC2)c1=O]

The title compound was prepared in a manner similar to that described in Example 33. ¹H NMR (500 MHz, DMSO) δ 12.31 (s, 1H), 10.21 (s, 1H), 8.76 (s, 1H), 8.49 (d, J=5.0 Hz, 1H), 8.25 (s, 1H), 8.06 (d, J=5.0 Hz, 1H), 7.10 (d, J=10.0 Hz, 2H), 7.07 (s, 1H), 6.64-6.61 (m, 1H), 6.53 (d, J=10.0 Hz, 2H), 6.07-6.01 (m, 1H), 5.24 (d, J=10.0 Hz, 1H), 5.12 (d, J=10.0 Hz, 1H), 4.74 (d, J=5.0 Hz, 2H), 4.35 (t, J=10.0 Hz, 1H), 3.93 (s, 3H), 3.68-3.65 (m, 2H), 3.57-3.44 (m, 7H), 3.27-3.26 (m, 2H), 2.68 (t, J=10.0 Hz, 2H), 2.40-2.33 (m, 4H), 2.19-2.07 (m, 6H), 1.96-1.85 (m, 4H), 1.69-1.59 (m, 1H), 1.15-1.08 (m, 2H). [M+H]⁺=790.6.

### Example 114: N-(2-((1r,4r)-4-(4-(1-(4-(2,4-dioxotetrahydropyrimidin-1(2H)-yl)-3,5-difluorophenyl)azetidine-3-carbonyl)piperazin-1-yl)cyclohexyl)-6-(2-hydroxypropan-2-yl)-2H-indazol-5-yl)-6-(trifluoromethyl)picolinamide

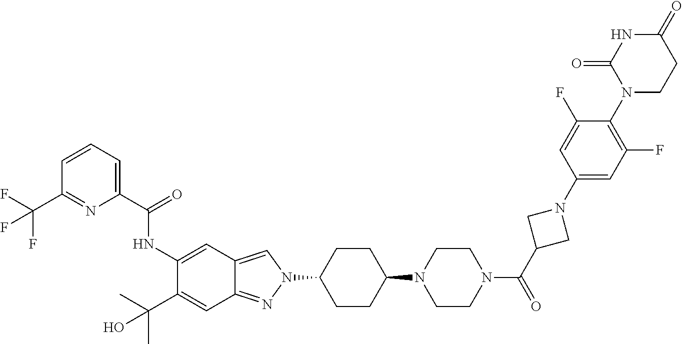 [SMILES: CC(C)(O)c1cc2nn([C@H]3CC[C@H](N4CCN(C(=O)C5CN(c6cc(F)c(N7CCC(=O)NC7=O)c(F)c6)C5)CC4)CC3)cc2cc1NC(=O)c1cccc(C(F)(F)F)n1]

The title compound was prepared in a manner similar to that described in Example 102. ¹H NMR (500 MHz, DMSO) δ 12.36 (s, 1H), 10.50 (s, 1H), 8.71 (s, 1H), 8.45 (d, J=10.0 Hz, 1H), 8.38-8.36 (m, 2H), 8.16 (d, J=10.0 Hz, 1H), 7.57 (s, 1H), 6.25 (d, J=10.0 Hz, 2H), 5.94 (s, 1H), 4.45-4.42 (m, 1H), 4.07-4.04 (m, 2H), 3.96-3.93 (m, 2H), 3.86-3.84 (m, 1H), 3.59 (t, J=5.0 Hz, 2H), 3.51-3.47 (m, 2H), 2.68 (t, J=5.0 Hz, 2H), 2.58-2.52 (m, 5H), 2.49-2.45 (m, 2H), 2.19-2.17 (m, 2H), 1.98-1.93 (m, 4H), 1.62 (s, 6H), 1.53-1.48 (m, 2H). [M+H]⁺=838.6.

### Example 115: N-(2-((1R,4r)-4-(4-((R)-1-(4-(2,4-dioxotetrahydropyrimidin-1(2H)-yl)-3,5-difluorophenyl)pyrrolidine-3-carbonyl)piperazin-1-yl)cyclohexyl)-6-(2-hydroxypropan-2-yl)-2H-indazol-5-yl)-6-(trifluoromethyl)picolinamide

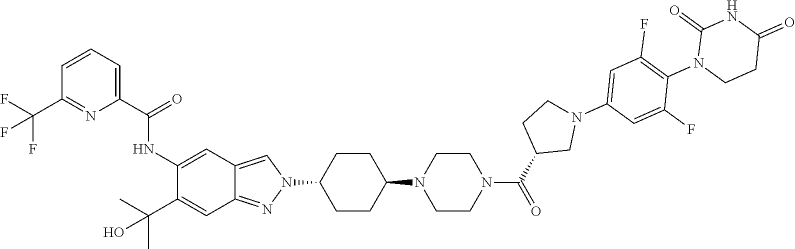 [SMILES: CC(C)(O)c1cc2nn([C@H]3CC[C@H](N4CCN(C(=O)[C@@H]5CCN(c6cc(F)c(N7CCC(=O)NC7=O)c(F)c6)C5)CC4)CC3)cc2cc1NC(=O)c1cccc(C(F)(F)F)n1]

The title compound was prepared in a manner similar to that described in Example 102.

¹H NMR (500 MHz, DMSO) δ 12.36 (s, 1H), 10.48 (s, 1H), 8.72 (s, 1H), 8.45 (d, J=10.0 Hz, 1H), 8.38-8.36 (m, 2H), 8.16 (d, J=10.0 Hz, 1H), 7.57 (s, 1H), 6.31 (d, J=10.0 Hz, 2H), 5.94 (s, 1H), 4.46-4.41 (m, 1H), 3.60-3.52 (m, 8H), 3.37-3.25 (m, 4H), 2.68 (t, J=5.0 Hz, 2H), 2.58-2.54 (m, 4H), 2.19-2.17 (m, 3H), 2.09-2.07 (m, 1H), 1.96-1.92 (m, 4H), 1.62 (s, 6H), 1.52-1.47 (m, 2H). [M+H]⁺=852.6.

### Example 171: 1-cyclopropyl-N-(2-((1R,4r)-4-((4-((R)-1-(4-(2,4-dioxotetrahydropyrimidin-1(2H)-yl)-3,5-difluorophenyl)pyrrolidine-3-carbonyl)piperazin-1-yl)methyl)cyclohexyl)-6-methoxy-2H-indazol-5-yl)-2-oxo-1,2-dihydropyridine-3-carboxamide

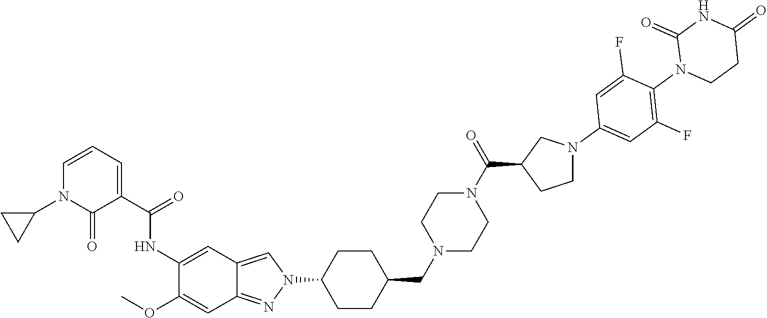 [SMILES: COc1cc2nn([C@H]3CC[C@H](CN4CCN(C(=O)[C@@H]5CCN(c6cc(F)c(N7CCC(=O)NC7=O)c(F)c6)C5)CC4)CC3)cc2cc1NC(=O)c1cccn(C2CC2)c1=O]

The title compound was prepared in a manner similar to that described in Example 102.

¹H NMR (500 MHz, DMSO) δ 12.31 (s, 1H), 10.47 (s, 1H), 8.76 (s, 1H), 8.48 (d, J=5.0 Hz, 1H), 8.25 (m, 1H), 8.05 (d, J=10.0 Hz, 1H), 7.07 (s, 1H), 6.64-6.61 (m, 1H), 6.31 (d, J=10.0 Hz, 2H), 6.05-6.04 (m, 1H), 5.25-5.10 (m, 2H), 4.73 (s, 2H), 4.36-4.34 (m, 1H), 3.93 (s, 3H), 2.40-2.32 (m, 4H), 2.19-2.05 (m, 7H), 1.96-1.85 (m, 4H), 1.68-1.55 (m, 2H), 1.15-1.10 (m, 2H). [M+H]⁺=827.3.

### Example 172: N-(2-((1R,4r)-4-(4-((R)-1-(4-((R)-2,6-dioxopiperidin-3-yl)-3,5-difluorophenyl)pyrrolidine-3-carbonyl)piperazin-1-yl)cyclohexyl)-6-(2-hydroxypropan-2-yl)-2H-indazol-5-yl)-8,8-difluoro-5,6,7,8-tetrahydroquinoline-2-carboxamide

### Step 1: 2-chloro-5,6,7,8-tetrahydroquinoline 1-oxide

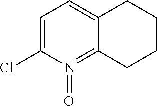 [SMILES: [O-][n+]1c(Cl)ccc2c1CCCC2]

To a DCM solution (30 mL) of 2-chloro-5,6,7,8-tetrahydroquinoline (2 g, 11.94 mmol) was added mCPBA (2.9 g, 14.33 mmol). The mixture was stirred overnight at room temperature. The solid was filtered and the filtrate was purified with combi flash, eluting with EA:PE=0:1˜1:0 to give the product (2.1 g, 95.8%). [M+H]⁺=184.3.

### Step 2: 2-chloro-5,6,7,8-tetrahydroquinolin-8-yl acetate

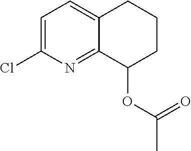

A solution of 2-chloro-5,6,7,8-tetrahydroquinoline 1-oxide (2.1 g, 11.44 mmol) in Ac₂O (50 mL) was stirred for 1 h at 120° C. under N₂. The solvent was removed under vacuum, and Na₂CO₃ aq. (30 mL) was added, then extracted with DCM (50 mL×2). The organic layer was purified with combi flash, eluted with EA:PE=0:1˜1:0 to afford the product (1.7 g, 66%). [M+H]⁺=226.3.

### Step 3: methyl 8-hydroxy-5,6,7,8-tetrahydroquinoline-2-carboxylate

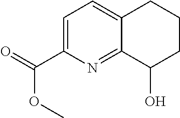

To a MeOH solution (20 mL) of 2-chloro-5,6,7,8-tetrahydroquinolin-8-yl acetate (1.7 g, 7.56 mmol) was added Pd(dppf)Cl₂·CH₂Cl₂ adduct (830 mg, 1.134 mmol) and Et₃N (2 mL). The mixture was stirred overnight at 90° C. under CO (4 atm). The mixture was concentrated and purified with combi flash, eluting with DCM:MeOH=1:0˜1:5 to afford the product (800 mg, 51%). [M+H]⁺=208.1.

### Step 4: methyl 8-oxo-5,6,7,8-tetrahydroquinoline-2-carboxylate

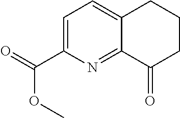

To a DMSO solution (10 mL) of methyl 8-hydroxy-5,6,7,8-tetrahydroquinoline-2-carboxylate (800 mg, 3.86 mmol) was added IBX (2.2 g, 7.72 mmol). The mixture was stirred overnight at room temperature, then H₂O (50 mL) was added, and extracted with DCM (50 mL×3). The organic layer was purified with combi flash, eluted with DCM:MeOH=1:0˜1:5 to afford the product (700 mg, 88%). [M+H]⁺=206.1.

### Step 5: methyl 8,8-difluoro-5,6,7,8-tetrahydroquinoline-2-carboxylate

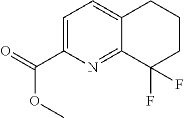 [SMILES: COC(=O)c1ccc2c(n1)C(F)(F)CCC2]

To a DCM solution (20 mL) of methyl 8-oxo-5,6,7,8-tetrahydroquinoline-2-carboxylate (700 mg, 3.41 mmol) was added Bis(2-Methoxyethyl)aminosulfur trifluoride (BAST) (3 mL) at 0° C. The mixture was stirred for 3 days at room temperature under N₂. The reaction was quenched with Na₂CO₃ aq. (50 mL) and extracted with DCM (50 mL×2). The organic layer was concentrated and purified with combi flash, eluted with DCM:MeOH=1:0˜1:5 to afford the product (700 mg, 90%). [M+H]⁺=228.1.

### Step 6: 8,8-difluoro-5,6,7,8-tetrahydroquinoline-2-carboxylic acid

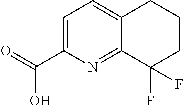 [SMILES: O=C(O)c1ccc2c(n1)C(F)(F)CCC2]

To a solution of methyl 8,8-difluoro-5,6,7,8-tetrahydroquinoline-2-carboxylate (700 mg, 3.07 mmol) in MeOH (20 mL) and H₂O (5 mL) was added NaOH (246 mg, 6.14 mmol). The mixture was stirred for 3 h at room temperature. The solvent was removed under vacuo. The residue was acidified to pH<7 with HCl aq., the solid was filtered and dried to yield the product (600 mg, 91%). [M+H]⁺=214.1.

### Step 7: methyl 2-((1r,4r)-4-(4-(tert-butoxycarbonyl)piperazin-1-yl)cyclohexyl)-5-(8,8-difluoro-5,6,7,8-tetrahydroquinoline-2-carboxamido)-2H-indazole-6-carboxylate

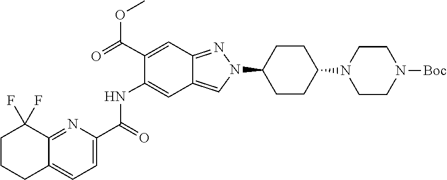 [SMILES: COC(=O)c1cc2nn([C@H]3CC[C@H](N4CCN(C(=O)OC(C)(C)C)CC4)CC3)cc2cc1NC(=O)c1ccc2c(n1)C(F)(F)CCC2]

To a DMF solution (3 mL) of 8,8-difluoro-5,6,7,8-tetrahydroquinoline-2-carboxylic acid (20 mg, 0.093 mmol) was added methyl 5-amino-2-((1r,4r)-4-(4-(tert-butoxycarbonyl)piperazin-1-yl)cyclohexyl)-2H-indazole-6-carboxylate (50 mg, 0.11 mmol), DIEA (0.5 mL) and HATU (53 mg, 0.14 mmol). The mixture was stirred overnight at room temperature. The mixture was purified with combi flash, eluted with DCM:MeOH=1-0 to 1:10 to afford the product (50 mg, 82%). [M+H]⁺=653.3.

### Step 8: tert-butyl 4-((1r,4r)-4-(5-(8,8-difluoro-5,6,7,8-tetrahydroquinoline-2-carboxamido)-6-(2-hydroxypropan-2-yl)-2H-indazol-2-yl)cyclohexyl)piperazine-1-carboxylate

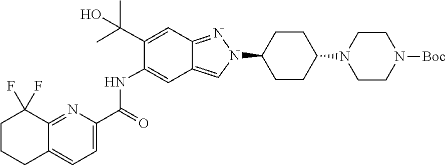 [SMILES: CC(C)(C)OC(=O)N1CCN([C@H]2CC[C@H](n3cc4cc(NC(=O)c5ccc6c(n5)C(F)(F)CCC6)c(C(C)(C)O)cc4n3)CC2)CC1]

To a solution of methyl 2-((1r,4r)-4-(4-(tert-butoxycarbonyl)piperazin-1-yl)cyclohexyl)-5-(8,8-difluoro-5,6,7,8-tetrahydroquinoline-2-carboxamido)-2H-indazole-6-carboxylate (50 mg, 0.077 mmol) in THF (10 mL) was added CH₃MgBr (3.2 N, 0.24 mL) at 0° C. under N₂. The mixture was stirred for 2 h at room temperature. The reaction was quenched with NH₄Cl aq. (50 mL) and extracted with DCM (50 mL×3). The organic layer was purified with combi flash, eluted with MeOH in DCM (0% to 10%) to afford the product (20 mg, 40%). [M+H]⁺=653.3.

### Step 9: 8,8-difluoro-N-(6-(2-hydroxypropan-2-yl)-2-((1r,4r)-4-(piperazin-1-yl)cyclohexyl)-2H-indazol-5-yl)-5,6,7,8-tetrahydroquinoline-2-carboxamide

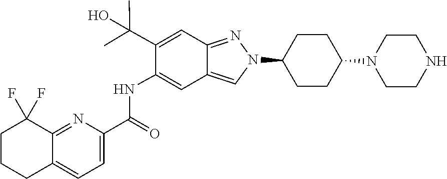 [SMILES: CC(C)(O)c1cc2nn([C@H]3CC[C@H](N4CCNCC4)CC3)cc2cc1NC(=O)c1ccc2c(n1)C(F)(F)CCC2]

To a DCM solution (10 mL) of tert-butyl 4-((1r,4r)-4-(5-(8,8-difluoro-5,6,7,8-tetrahydroquinoline-2-carboxamido)-6-(2-hydroxypropan-2-yl)-2H-indazol-2-yl)cyclohexyl)piperazine-1-carboxylate (20 mg, 0.31 mmol) was added TFA (3 mL). The mixture was stirred overnight at room temperature. The solvent was removed under vacuum. The residue was basified to pH>7 with Na₂CO₃ aq., extracted with DCM (50 mL×3). The organic layer was dried over Na₂SO₄, filtered and concentrated to afford crude product (13 mg, 77%). [M+H]⁺=553.3.

### Step 10: N-(2-((1R,4r)-4-(4-((R)-1-(4-((R)-2,6-dioxopiperidin-3-yl)-3,5-difluorophenyl)pyrrolidine-3-carbonyl)piperazin-1-yl)cyclohexyl)-6-(2-hydroxypropan-2-yl)-2H-indazol-5-yl)-8,8-difluoro-5,6,7,8-tetrahydroquinoline-2-carboxamide

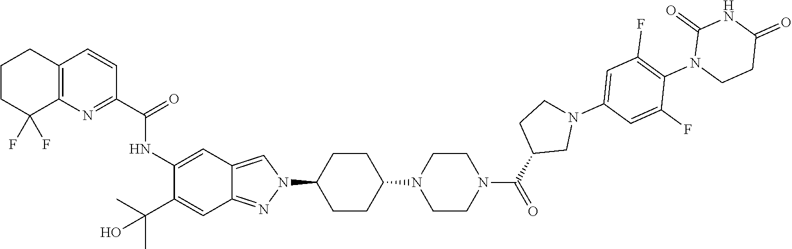 [SMILES: CC(C)(O)c1cc2nn([C@H]3CC[C@H](N4CCN(C(=O)[C@@H]5CCN(c6cc(F)c(N7CCC(=O)NC7=O)c(F)c6)C5)CC4)CC3)cc2cc1NC(=O)c1ccc2c(n1)C(F)(F)CCC2]

To a DMF solution (5 mL) of 8,8-difluoro-N-(6-(2-hydroxypropan-2-yl)-2-((1r,4r)-4-(piperazin-1-yl)cyclohexyl)-2H-indazol-5-yl)-5,6,7,8-tetrahydroquinoline-2-carboxamide (13 mg, 0.024 mmol) was added (R)-1-(4-((R)-2,6-dioxopiperidin-3-yl)-3,5-difluorophenyl)pyrrolidine-3-carboxylic acid (10 mg, 0.0288 mmol), DIEA (0.5 mL) and HATU (14 mg, 0.036 mmol). The mixture was stirred overnight at room temperature. The mixture was purified with combi flash, eluted with DCM:MeOH=1-0 to 1:10 to yield the crude product which was further purified with prep. HPLC to afford the product (11 mg, 54%). ¹H NMR (500 MHz, DMSO) δ 12.17 (s, 1H), 10.84 (s, 1H), 8.64 (s, 1H), 8.34 (s, 1H), 8.19 (d, J=10.0 Hz, 1H), 7.97 (d, J=10.0 Hz, 1H), 7.55 (s, 1H), 6.23 (d, J=10.0 Hz, 2H), 5.79 (s, 1H), 4.43 (t, J=10.0 Hz, 1H), 4.04-4.00 (m, 1H), 3.55-3.42 (m, 9H), 3.28-3.24 (m, 2H), 2.97 (s, 2H), 2.78-2.74 (m, 2H), 2.58 (s, 3H), 2.44-2.40 (m, 2H), 2.17-2.07 (m, 6H), 1.96-1.91 (m, 7H), 1.60 (s, 6H), 1.52-1.49 (m, 2H). [M+H]⁺=873.4.

### Example 173: N-(2-((1r,4r)-4-(4-(1-(4-(2,4-dioxotetrahydropyrimidin-1(2H)-yl)-3,5-difluorophenyl)azetidine-3-carbonyl)piperazin-1-yl)cyclohexyl)-6-(methoxy-d3)-2H-indazol-5-yl)-6-(trifluoromethyl)picolinamide

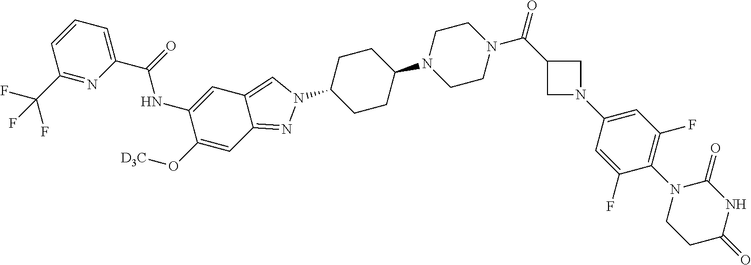 [SMILES: [2H]C([2H])([2H])Oc1cc2nn([C@H]3CC[C@H](N4CCN(C(=O)C5CN(c6cc(F)c(N7CCC(=O)NC7=O)c(F)c6)C5)CC4)CC3)cc2cc1NC(=O)c1cccc(C(F)(F)F)n1]

The title compound was prepared in a manner similar to that described in Example 164.

¹H NMR (500 MHz, DMSO) δ 10.50 (s, 2H), 8.69 (s, 1H), 8.45 (d, J=10.0 Hz, 1H), 8.42-8.39 (m, 1H), 8.33 (s, 1H), 8.23 (d, J=10.0 Hz, 1H), 7.15 (s, 1H), 6.25 (d, J=10.0 Hz, 2H), 4.39-4.37 (m, 1H), 4.07-4.04 (m, 2H), 3.96-3.93 (m, 2H), 3.86-3.83 (m, 1H), 3.59 (t, J=5.0 Hz, 2H), 3.51-3.47 (m, 2H), 2.68 (t, J=5.0 Hz, 2H), 2.58-2.42 (m, 7H), 2.19-2.17 (m, 2H), 1.94-1.89 (m, 4H), 1.53-1.48 (m, 2H). [M+H]⁺=813.3.

### Example 174: N-(2-((1R,4r)-4-(4-((R)-1-(4-(2,4-dioxotetrahydropyrimidin-1(2H)-yl)-3,5-difluorophenyl)pyrrolidine-3-carbonyl)piperazin-1-yl)cyclohexyl)-6-(methoxy-d3)-2H-indazol-5-yl)-6-(trifluoromethyl)picolinamide

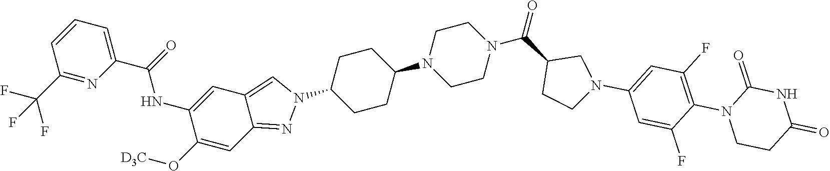 [SMILES: [2H]C([2H])([2H])Oc1cc2nn([C@H]3CC[C@H](N4CCN(C(=O)[C@@H]5CCN(c6cc(F)c(N7CCC(=O)NC7=O)c(F)c6)C5)CC4)CC3)cc2cc1NC(=O)c1cccc(C(F)(F)F)n1]

The title compound was prepared in a manner similar to that described in Example 164.

¹H NMR (500 MHz, DMSO) δ 10.54-10.48 (m, 2H), 8.69 (s, 1H), 8.46 (d, J=10.0 Hz, 1H), 8.42-8.39 (m, 1H), 8.33 (s, 1H), 8.22 (d, J=10.0 Hz, 1H), 7.15 (s, 1H), 6.32 (d, J=10.0 Hz, 2H), 4.39-4.37 (m, 1H), 3.60-3.45 (m, 7H), 3.38-3.27 (m, 4H), 2.69 (t, J=5.0 Hz, 2H), 2.58-2.42 (m, 6H), 2.19-2.07 (m, 4H), 1.95-1.89 (m, 3H), 1.53-1.48 (m, 2H). [M+H]⁺=827.6.

### Example 176: N-(2-((1r,4r)-4-(4-(1-(4-(2,4-dioxotetrahydropyrimidin-1(2H)-yl)-3,5-difluorophenyl)azetidine-3-carbonyl)piperazin-1-yl)cyclohexyl)-6-methoxy-2H-indazol-5-yl)-6-(trifluoromethyl)picolinamide

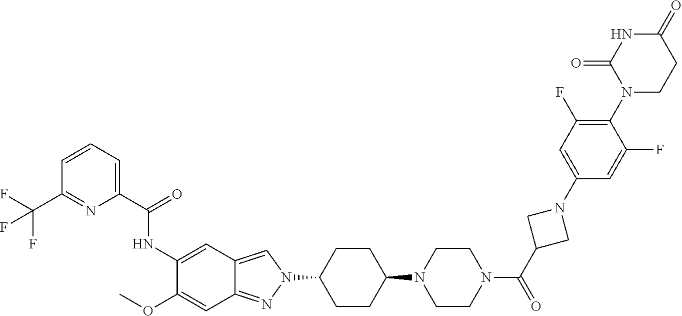 [SMILES: COc1cc2nn([C@H]3CC[C@H](N4CCN(C(=O)C5CN(c6cc(F)c(N7CCC(=O)NC7=O)c(F)c6)C5)CC4)CC3)cc2cc1NC(=O)c1cccc(C(F)(F)F)n1]

The title compound was prepared in a manner similar to that described in Example 164.

¹H NMR (500 MHz, DMSO) δ 10.50 (s, 2H), 8.69 (s, 1H), 8.46 (d, J=10.0 Hz, 1H), 8.42-8.39 (m, 1H), 8.33 (s, 1H), 8.22 (d, J=10.0 Hz, 1H), 7.15 (s, 1H), 6.25 (d, J=10.0 Hz, 2H), 4.38-4.35 (m, 1H), 4.07-4.04 (m, 2H), 3.96-3.93 (m, 5H), 3.85-3.83 (m, 1H), 3.59 (t, J=5.0 Hz, 2H), 3.49 (m, 2H), 2.68 (t, J=5.0 Hz, 2H), 2.58-2.45 (m, 7H), 2.19-2.17 (m, 2H), 1.95-1.89 (m, 4H), 1.53-1.48 (m, 2H). [M+H]⁺=810.6.

### Example 177: N-(2-((1R,4r)-4-(4-((R)-1-(4-(2,4-dioxotetrahydropyrimidin-1(2H)-yl)-3,5-difluorophenyl)pyrrolidine-3-carbonyl)piperazin-1-yl)cyclohexyl)-6-methoxy-2H-indazol-5-yl)-6-(trifluoromethyl)picolinamide

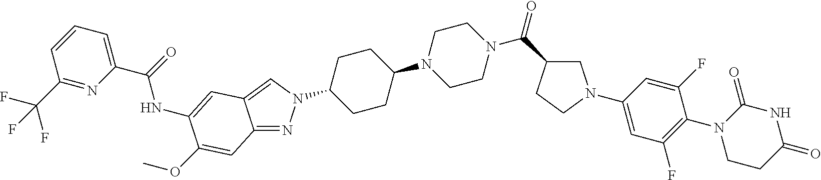 [SMILES: COc1cc2nn([C@H]3CC[C@H](N4CCN(C(=O)[C@@H]5CCN(c6cc(F)c(N7CCC(=O)NC7=O)c(F)c6)C5)CC4)CC3)cc2cc1NC(=O)c1cccc(C(F)(F)F)n1]

The title compound was prepared in a manner similar to that described in Example 164. ¹H NMR (500 MHz, DMSO) δ 10.50-10.48 (s, 2H), 8.69 (s, 1H), 8.46 (d, J=10.0 Hz, 1H), 8.42-8.39 (m, 1H), 8.33 (s, 1H), 8.22 (d, J=10.0 Hz, 1H), 7.16 (s, 1H), 6.32 (d, J=10.0 Hz, 2H), 4.44-4.35 (m, 1H), 3.98 (s, 3H), 3.60-3.47 (m, 7H), 3.38-3.27 (m, 4H), 2.69 (t, J=5.0 Hz, 2H), 2.58-2.45 (m, 6H), 2.19-2.10 (m, 4H), 1.98-1.89 (m, 3H), 1.53-1.48 (m, 2H). [M+H]⁺=824.7.

### Example 346: (R)-3-cyano-N-(2-(1′-(1-(4-(2,6-dioxopiperidin-3-yl)-3,5-difluorophenyl)azetidine-3-carbonyl)-[1,4′-bipiperidin]-4-yl)-6-(2-hydroxypropan-2-yl)-2H-indazol-5-yl)pyrrolo[1,2-b]pyridazine-7-carboxamide

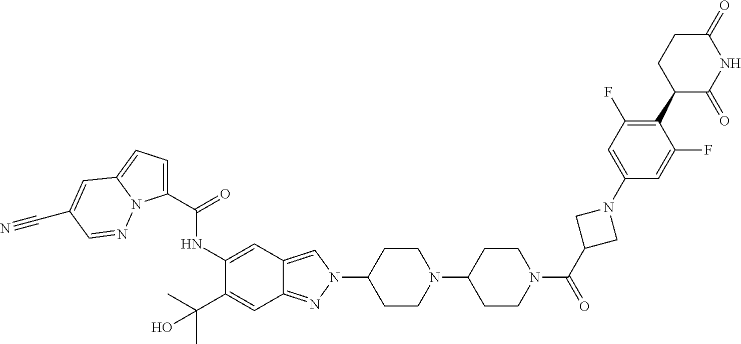 [SMILES: CC(C)(O)c1cc2nn(C3CCN(C4CCN(C(=O)C5CN(c6cc(F)c([C@H]7CCC(=O)NC7=O)c(F)c6)C5)CC4)CC3)cc2cc1NC(=O)c1ccc2cc(C#N)cnn12]

The title compound was prepared in a manner similar to that described in Example 178. ¹H NMR (500 MHz, DMSO) δ 12.03 (s, 1H), 10.86 (s, 1H), 8.94 (d, J=5 Hz, 1H), 8.74 (d, J=5 Hz, 1H), 8.56 (s, 1H), 8.38 (s, 1H), 7.72 (d, J=5 Hz, 1H), 7.58 (s, 1H), 7.09 (d, J=5 Hz, 1H), 6.17 (d, J=10 Hz, 2H), 5.73 (s, 1H), 4.46-4.38 (m, 2H), 4.07-4.00 (m, 3H), 3.95-3.86 (m, 2H), 3.85-3.81 (m, 1H), 3.04-2.95 (m, 3H), 2.82-2.73 (m, 1H), 2.64-2.53 (m, 2H), 2.48-2.33 (m, 4H), 2.14-2.02 (m, 4H), 1.98-1.90 (m, 1H), 1.82-1.75 (m, 2H), 1.63 (s, 6H), 1.45-1.37 (m, 1H), 1.35-1.27 (m, 1H). [M+H]⁺=833.6.

### Example 347: 3-cyano-N-(2-(1′-((R)-1-(4-((R)-2,6-dioxopiperidin-3-yl)-3,5-difluorophenyl)pyrrolidine-3-carbonyl)-[1,4′-bipiperidin]-4-yl)-6-(2-hydroxypropan-2-yl)-2H-indazol-5-yl)pyrrolo[1,2-b]pyridazine-7-carboxamide

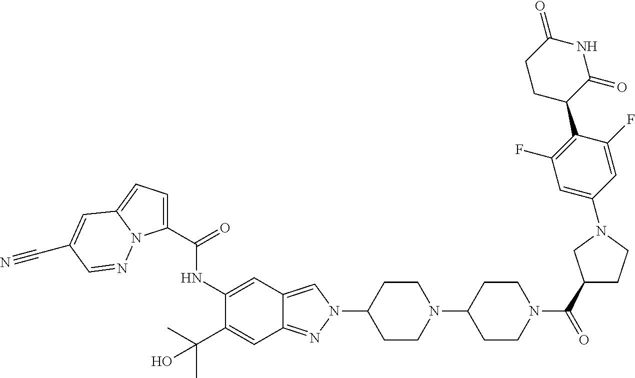 [SMILES: CC(C)(O)c1cc2nn(C3CCN(C4CCN(C(=O)[C@@H]5CCN(c6cc(F)c([C@H]7CCC(=O)NC7=O)c(F)c6)C5)CC4)CC3)cc2cc1NC(=O)c1ccc2cc(C#N)cnn12]

The title compound (14.67 mg, 34.7%) was prepared in a manner similar to that described in Example 178. ¹H NMR (500 MHz, DMSO) δ 12.03 (s, 1H), 10.84 (s, 1H), 8.93 (d, J=5 Hz, 1H), 8.74 (d, J=5 Hz, 1H), 8.56 (s, 1H), 8.38 (s, 1H), 7.72 (d, J=5 Hz, 1H), 7.58 (s, 1H), 7.09 (d, J=5 Hz, 1H), 6.23 (t, J=10 Hz, 2H), 5.73 (s, 1H), 4.48-4.37 (m, 2H), 4.10-3.98 (m, 2H), 3.57-3.51 (m, 2H), 3.47-3.41 (m, 2H), 3.29-3.22 (m, 2H), 3.10-2.98 (m, 4H), 2.83-2.74 (m, 2H), 2.65-2.55 (m, 2H), 2.45-2.34 (m, 2H), 2.20-2.02 (m, 5H), 1.97-1.91 (m, 1H), 1.86-1.75 (m, 2H), 1.63 (s, 6H), 1.52-1.37 (m, 1H), 1.35-1.23 (m, 1H). [M+H]⁺=847.8.

### Example 187: 3-cyano-N-(2-((1r,4r)-4-(4-(1-(6-(2,6-dioxopiperidin-3-yl)-5-fluoropyridin-3-yl)azetidine-3-carbonyl)piperazin-1-yl)cyclohexyl)-6-(2-hydroxypropan-2-yl)-2H-indazol-5-yl)pyrrolo[1,2-b]pyridazine-7-carboxamide

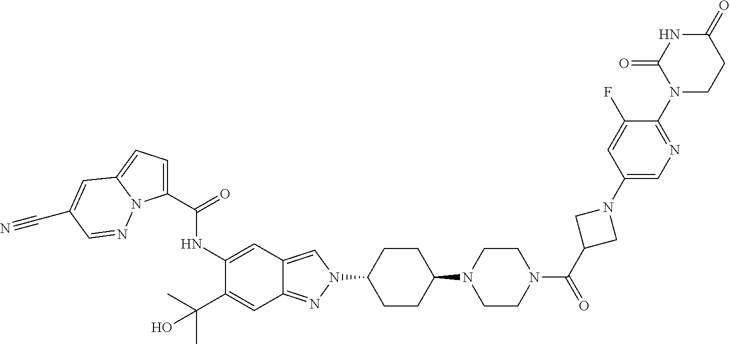 [SMILES: CC(C)(O)c1cc2nn([C@H]3CC[C@H](N4CCN(C(=O)C5CN(c6cnc(N7CCC(=O)NC7=O)c(F)c6)C5)CC4)CC3)cc2cc1NC(=O)c1ccc2cc(C#N)cnn12]

The title compound (96.33 mg, 62.2%) was prepared in a manner similar to that described in Example 178. ¹H NMR (500 MHz, DMSO) δ 12.02 (s, 1H), 10.84 (s, 1H), 8.93 (d, J=5 Hz, 1H), 8.74 (d, J=5 Hz, 1H), 8.55 (s, 1H), 8.34 (s, 1H), 7.72 (d, J=5 Hz, 1H), 7.64 (s, 1H), 7.57 (s, 1H), 7.09 (d, J=5 Hz, 1H), 6.82 (d, J=10 Hz, 2H), 5.73 (s, 1H), 4.47-4.40 (m, 1H), 4.13-4.06 (m, 3H), 3.99 (t, J=5 Hz, 2H), 3.92-3.85 (m, 1H), 3.52-3.45 (m, 2H), 3.36-3.33 (m, 4H), 2.72-2.63 (m, 1H), 2.57-2.51 (m, 4H), 2.25-2.15 (m, 3H), 2.07-1.90 (m, 5H), 1.63 (s, 6H), 1.56-1.45 (m, 2H). [M+H]⁺=816.6.

### Example 212: 3-cyano-N-(2-((1R,4r)-4-(4-((R)-1-(4-(2,4-dioxotetrahydropyrimidin-1(2H)-yl)-3-fluoro-5-methoxyphenyl)pyrrolidine-3-carbonyl)piperazin-1-yl)cyclohexyl)-6-(2-hydroxypropan-2-yl)-2H-indazol-5-yl)pyrrolo[1,2-b]pyridazine-7-carboxamide

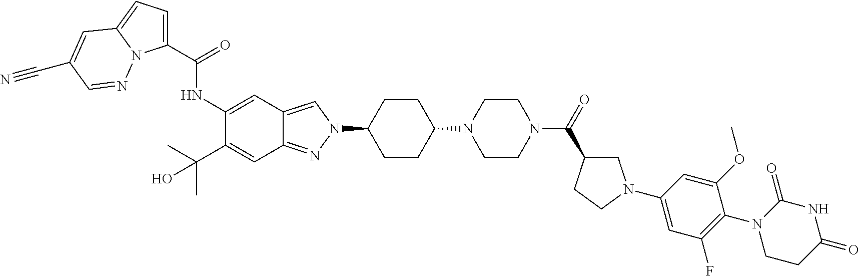 [SMILES: COc1cc(N2CC[C@@H](C(=O)N3CCN([C@H]4CC[C@H](n5cc6cc(NC(=O)c7ccc8cc(C#N)cnn78)c(C(C)(C)O)cc6n5)CC4)CC3)C2)cc(F)c1N1CCC(=O)NC1=O]

The title compound (86.48 mg, 53%) was prepared in a manner similar to that described in Example 178. ¹H NMR (500 MHz, DMSO) δ 12.02 (s, 1H), 10.29 (s, 1H), 8.93 (d, J=5 Hz, 1H), 8.74 (d, J=5 Hz, 1H), 8.55 (s, 1H), 8.34 (s, 1H), 7.72 (d, J=5 Hz, 1H), 7.57 (s, 1H), 7.09 (d, J=5 Hz, 1H), 6.03-5.98 (m, 2H), 5.72 (s, 1H), 4.47-4.40 (m, 1H), 3.80 (s, 3H), 3.60-3.43 (m, 8H), 3.42-3.35 (m, 2H), 3.31-3.25 (m, 2H), 2.66-2.55 (m, 4H), 2.54-2.51 (m, 2H), 2.55-2.02 (m, 4H), 2.01-1.90 (m, 4H), 1.63 (s, 6H), 1.56-1.46 (m, 2H). [M+H]⁺=860.7.

### Example 289: 3-cyano-N-(2-((1r,4r)-4-(4-(1-(4-((R)-2,6-dioxopiperidin-3-yl)-3,5-dimethylphenyl)piperidin-4-yl)piperazin-1-yl)cyclohexyl)-6-(2-hydroxypropan-2-yl)-2H-indazol-5-yl)pyrrolo[1,2-b]pyridazine-7-carboxamide

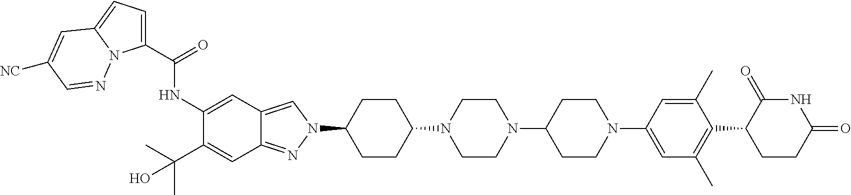 [SMILES: Cc1cc(N2CCC(N3CCN([C@H]4CC[C@H](n5cc6cc(NC(=O)c7ccc8cc(C#N)cnn78)c(C(C)(C)O)cc6n5)CC4)CC3)CC2)cc(C)c1[C@H]1CCC(=O)NC1=O]

The title compound (17.63 mg, 21.4%) was prepared in a manner similar to that described in Example 290. ¹H NMR (500 MHz, DMSO) δ 12.02 (s, 1H), 10.76 (s, 1H), 8.93 (d, J=5 Hz, 1H), 8.74 (d, J=5 Hz, 1H), 8.55 (s, 1H), 8.33 (s, 1H), 8.16 (s, 1H), 7.72 (d, J=5 Hz, 1H), 7.57 (s, 1H), 7.09 (d, J=5 Hz, 1H), 6.61 (s, 1H), 6.56 (s, 1H), 5.72 (s, 1H), 4.46-4.38 (m, 1H), 4.02 (dd, J=10, 5 Hz, 1H), 3.71-3.65 (m, 2H), 2.83-2.74 (m, 1H), 2.65-2.52 (m, 10H), 2.48-2.35 (m, 2H), 2.33-2.23 (m, 4H), 2.20-2.14 (m, 2H), 2.11-2.01 (m, 4H), 2.00-1.89 (m, 4H), 1.87-1.80 (m, 3H), 1.63 (s, 6H), 1.52-1.42 (m, 4H). [M+H]⁺=−825.6

### Example 292: 3-cyano-N-(2-((1r,4r)-4-(4-(4-(4-(2,6-dioxopiperidin-3-yl)phenyl)piperazine-1-carbonyl)piperazin-1-yl)cyclohexyl)-6-(2-hydroxypropan-2-yl)-2H-indazol-5-yl)pyrrolo[1,2-b]pyridazine-7-carboxamide

### Step 1: tert-butyl 4-(4-(2,6-bis(benzyloxy)pyridin-3-yl)phenyl)piperazine-1-carboxylate

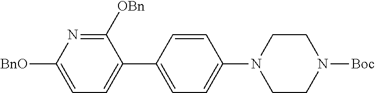 [SMILES: CC(C)(C)OC(=O)N1CCN(c2ccc(-c3ccc(OCc4ccccc4)nc3OCc3ccccc3)cc2)CC1]

To a solution of 2,6-bis(benzyloxy)-3-bromopyridine (3.69 g, 10 mmol) and tert-butyl 4-(4-(4,4,5,5-tetramethyl-1,3,2-dioxaborolan-2-yl)phenyl)piperazine-1-carboxylate (4.656 g, 12 mmol) in 1,4-dioxane (40 mL) and water (10 mL) was added K₂CO₃ (2.76 g, 20 mmol) and Pd(dppf)Cl₂ (0.732 g, 1 mmol). The mixture was stirred for 2 hrs at 100° C. under N₂. The mixture was cooled to rt and concentrated in vacuo. The residue was purified by silica gel column chromatography (PE:EA=3:1) to give product (4.8 g, 87.1%). [M+H]⁺=552.3.

### Step 2: tert-butyl 4-(4-(2,6-dioxopiperidin-3-yl)phenyl)piperazine-1-carboxylate

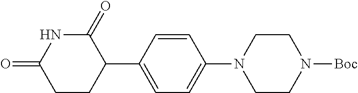 [SMILES: CC(C)(C)OC(=O)N1CCN(c2ccc(C3CCC(=O)NC3=O)cc2)CC1]

To a solution of tert-butyl 4-(4-(2,6-bis(benzyloxy)pyridin-3-yl)phenyl)piperazine-1-carboxylate (4.8 g, 8.71 mmol) in DMF (40 mL) and IPA (40 mL) was added Pd/C (1 g) at rt. To the above solution was introduce into H₂ (g). The mixture was stirred overnight at 45° C. The mixture was filtered. The filtrate was evaporated in vacuum to afford product (2.4 g, 73.8%), which was used without further purification. [M+H]⁺=374.2

### Step 3: 3-(4-(piperazin-1-yl)phenyl)piperidine-2,6-dione

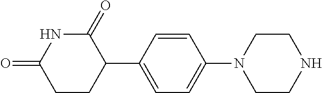 [SMILES: O=C1CCC(c2ccc(N3CCNCC3)cc2)C(=O)N1]

To a stirred solution of tert-butyl 4-(4-(2,6-dioxopiperidin-3-yl)phenyl)piperazine-1-carboxylate (2.4 g, 6.43 mmol) in DCM (30 mL) was added TFA (5 mL). The reaction mixture was stirred 1 h at rt. The mixture was diluted with water, adjusted to PH 8 with sat.NaHCO₃ solution and extracted with DCM. The combined organic layers were dried over anhydrous Na₂SO₄, filtered and evaporated in vacuum to give product (1.5 g, 85.3%). [M+H]⁺=274.1.

### Step 4: 4-(4-(2,6-dioxopiperidin-3-yl)phenyl)piperazine-1-carbonyl chloride

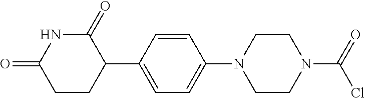 [SMILES: O=C1CCC(c2ccc(N3CCN(C(=O)Cl)CC3)cc2)C(=O)N1]

To a solution of triphosgene (89.1 mg, 0.3 mmol) and Et₃N (151.5 mg, 1.5 mmol) in DCM (10 mL) was added 3-(4-(piperazin-1-yl)phenyl)piperidine-2,6-dione (246.6 mg, 0.9 mmol) at 0° C. The resulting mixture was stirred overnight at rt. The mixture was diluted with water and extracted with DCM. The combined organic layers were dried over anhydrous Na₂SO₄, filtered and evaporated in vacuum to give product (145 mg, 48%). [M+H]⁺=336.1.

### Step 5: 3-cyano-N-(2-((1r,4r)-4-(4-(4-(4-(2,6-dioxopiperidin-3-yl)phenyl)piperazine-1-carbonyl)piperazin-1-yl)cyclohexyl)-6-(2-hydroxypropan-2-yl)-2H-indazol-5-yl)pyrrolo[1,2-b]pyridazine-7-carboxamide

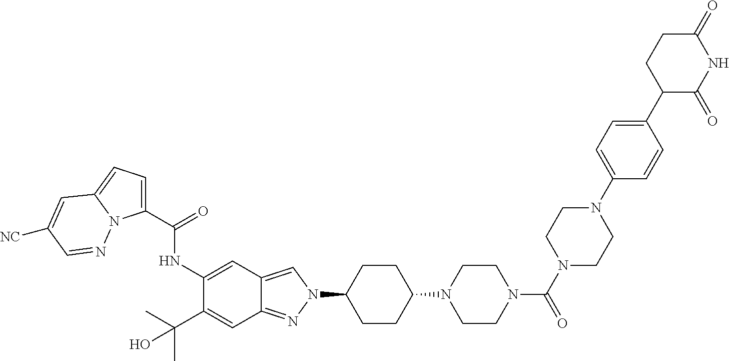 [SMILES: CC(C)(O)c1cc2nn([C@H]3CC[C@H](N4CCN(C(=O)N5CCN(c6ccc(C7CCC(=O)NC7=O)cc6)CC5)CC4)CC3)cc2cc1NC(=O)c1ccc2cc(C#N)cnn12]

To a solution of 3-cyano-N-(6-(2-hydroxypropan-2-yl)-2-((1r,4r)-4-(piperazin-1-yl)cyclohexyl)-2H-indazol-5-yl)pyrrolo[1,2-b]pyridazine-7-carboxamide (52.6 mg, 0.1 mmol) and Et₃N (30.3 mg, 0.3 mmol) in DCM (10 mL) was added 4-(4-(2,6-dioxopiperidin-3-yl)phenyl)piperazine-1-carbonyl chloride (50.4 mg, 0.15 mmol) at 0° C. The resulting mixture was stirred for 1 h at 0° C. The mixture was diluted with water and extracted with DCM. The combined organic layers were dried over anhydrous Na₂SO₄, filtered and evaporated in vacuum. The residue was purified by Prep-HPLC (C-18 column chromatography (0.1% FA in water:acetonitrile=90:10˜60:40 gradient elution) to give the desired product (37.93 mg, 45.9%). ¹H NMR (500 MHz, DMSO) δ 12.02 (s, 1H), 10.78 (s, 1H), 8.93 (d, J=5 Hz, 1H), 8.74 (d, J=5 Hz, 1H), 8.55 (s, 1H), 8.34 (s, 1H), 7.72 (d, J=5 Hz, 1H), 7.57 (s, 1H), 7.10-7.05 (m, 3H), 6.92 (d, J=10 Hz, 2H), 5.72 (s, 1H), 4.46-4.38 (m, 1H), 3.74 (dd, J=10, 5 Hz, 1H), 3.22-3.17 (m, 4H), 3.14-3.08 (m, 4H), 2.67-2.59 (m, 2H), 2.57-2.53 (m, 4H), 2.48-2.43 (m, 1H), 2.22-2.10 (m, 3H), 2.04-1.90 (m, 5H), 1.63 (s, 6H), 1.55-1.44 (m, 2H). [M+H]⁺=826.4.

### Example 225: 3-cyano-N-(2-((1r,4r)-4-(4-(4-(4-((R)-2,6-dioxopiperidin-3-yl)-3,5-difluorophenyl)piperazine-1-carbonyl)piperazin-1-yl)cyclohexyl)-6-(2-hydroxypropan-2-yl)-2H-indazol-5-yl)pyrrolo[1,2-b]pyridazine-7-carboxamide

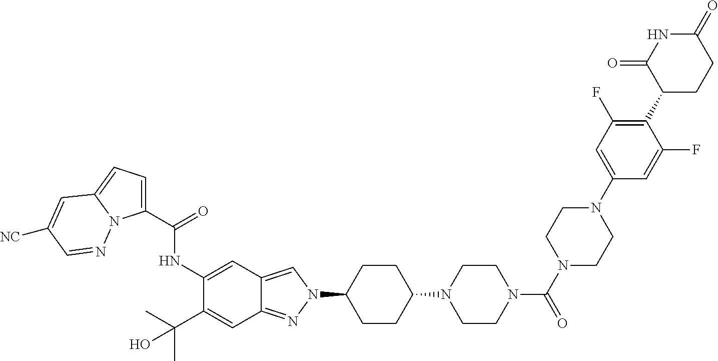 [SMILES: CC(C)(O)c1cc2nn([C@H]3CC[C@H](N4CCN(C(=O)N5CCN(c6cc(F)c([C@H]7CCC(=O)NC7=O)c(F)c6)CC5)CC4)CC3)cc2cc1NC(=O)c1ccc2cc(C#N)cnn12]

The title compound (57.64 mg, 66.9%) was prepared in a manner similar to that described in Example 292. ¹H NMR (500 MHz, DMSO) δ 12.02 (s, 1H), 10.88 (s, 1H), 8.93 (d, J=5 Hz, 1H), 8.74 (d, J=5 Hz, 1H), 8.55 (s, 1H), 8.34 (s, 1H), 7.72 (d, J=5 Hz, 1H), 7.57 (s, 1H), 7.09 (d, J=5 Hz, 1H), 6.66 (d, J=15 Hz, 2H), 5.72 (s, 1H), 4.46-4.39 (m, 1H), 4.06 (dd, J=10, 5 Hz, 1H), 3.28-3.17 (m, 12H), 2.83-2.74 (m, 1H), 2.56-2.52 (m, 5H), 2.47-2.43 (m, 1H), 2.22-2.04 (m, 3H), 2.00-1.90 (m, 5H), 1.63 (s, 6H), 1.55-1.44 (m, 2H). [M+H]⁺=862.4.

### Example 294 and Example 295: 3-cyano-N-(2-((1R,4r)-4-(4-((R)-1-(4-(2,4-dioxotetrahydropyrimidin-1(2H)-yl)phenyl)pyrrolidin-3-yl)piperazin-1-yl)cyclohexyl)-6-(2-hydroxypropan-2-yl)-2H-indazol-5-yl)pyrrolo[1,2-b]pyridazine-7-carboxamide and 3-cyano-N-(2-((1S,4r)-4-(4-((S)-1-(4-(2,4-dioxotetrahydropyrimidin-1(2H)-yl)phenyl)pyrrolidin-3-yl)piperazin-1-yl)cyclohexyl)-6-(2-hydroxypropan-2-yl)-2H-indazol-5-yl)pyrrolo[1,2-b]pyridazine-7-carboxamide

### Step 1: 3-cyano-N-(2-((1r,4r)-4-(4-(1-(4-(2,4-dioxotetrahydropyrimidin-1(2H)-yl)phenyl)pyrrolidin-3-yl)piperazin-1-yl)cyclohexyl)-6-(2-hydroxypropan-2-yl)-2H-indazol-5-yl)pyrrolo[1,2-b]pyridazine-7-carboxamide

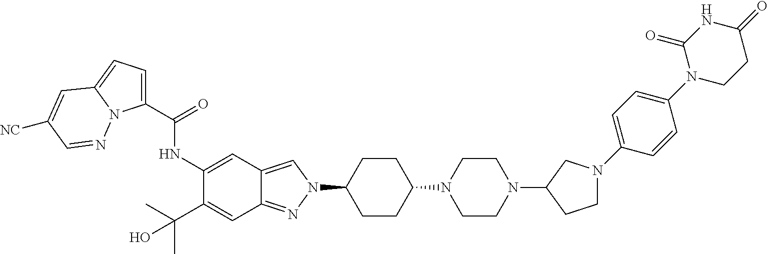 [SMILES: CC(C)(O)c1cc2nn([C@H]3CC[C@H](N4CCN(C5CCN(c6ccc(N7CCC(=O)NC7=O)cc6)C5)CC4)CC3)cc2cc1NC(=O)c1ccc2cc(C#N)cnn12]

The title compound (80 mg, 25.5%). was prepared in a manner similar to that described in Example 290. [M+H]⁺=784.4.

### Step 2: 3-cyano-N-(2-((1R,4r)-4-(4-((R)-1-(4-(2,4-dioxotetrahydropyrimidin-1(2H)-yl)phenyl)pyrrolidin-3-yl)piperazin-1-yl)cyclohexyl)-6-(2-hydroxypropan-2-yl)-2H-indazol-5-yl)pyrrolo[1,2-b]pyridazine-7-carboxamide and 3-cyano-N-(2-((1S,4r)-4-(4-((S)-1-(4-(2,4-dioxotetrahydropyrimidin-1(2H)-yl)phenyl)pyrrolidin-3-yl)piperazin-1-yl)cyclohexyl)-6-(2-hydroxypropan-2-yl)-2H-indazol-5-yl)pyrrolo[1,2-b]pyridazine-7-carboxamide

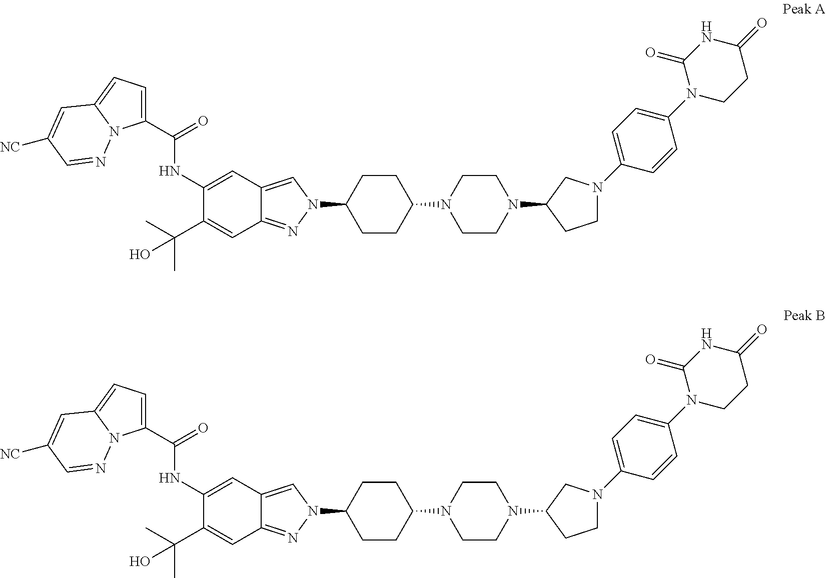 [SMILES: CC(C)(O)c1cc2nn([C@H]3CC[C@H](N4CCN([C@@H]5CCN(c6ccc(N7CCC(=O)NC7=O)cc6)C5)CC4)CC3)cc2cc1NC(=O)c1ccc2cc(C#N)cnn12 ; CC(C)(O)c1cc2nn([C@H]3CC[C@H](N4CCN([C@H]5CCN(c6ccc(N7CCC(=O)NC7=O)cc6)C5)CC4)CC3)cc2cc1NC(=O)c1ccc2cc(C#N)cnn12]

The 3-cyano-N-(2-((1r,4r)-4-(4-(1-(4-(2,4-dioxotetrahydropyrimidin-1(2H)-yl)phenyl)pyrrolidin-3-yl)piperazin-1-yl)cyclohexyl)-6-(2-hydroxypropan-2-yl)-2H-indazol-5-yl)pyrrolo[1,2-b]pyridazine-7-carboxamide (80 mg, 0.102 mmol) was separated by Chiral-HPLC on a CHIRALPAK IA with Hexane:EtOH=70:30 as an eluent at a flow rate of 20 mL/min. After chiral separation by HPLC under neutral condition, peak A (27.76 mg, 34.7%) and peak B (26.63 mg, 33.3%) were obtained respectively.

Peak A: ¹H NMR (500 MHz, DMSO) δ 12.02 (s, 1H), 10.21 (s, 1H), 8.93 (d, J=5 Hz, 1H), 8.74 (d, J=5 Hz, 1H), 8.55 (s, 1H), 8.33 (s, 1H), 7.72 (d, J=5 Hz, 1H), 7.57 (s, 1H), 7.12-7.04 (m, 3H), 6.53 (d, J=10 Hz, 2H), 5.72 (s, 1H), 4.46-4.39 (m, 1H), 3.66 (t, J=5 Hz, 2H), 3.46-3.44 (m, 1H), 3.37-3.34 (m, 1H), 3.30-3.27 (m, 1H), 3.07-3.03 (m, 1H), 2.96-2.85 (m, 1H), 2.68 (t, J=5 Hz, 2H), 2.65-2.52 (m, 5H), 2.48-2.35 (m, 3H), 2.22-2.12 (m, 3H), 2.02-1.90 (m, 4H), 1.86-1.77 (m, 1H), 1.63 (s, 6H), 1.54-1.43 (m, 2H). [M+H]⁺=784.7

Peak B: ¹H NMR (500 MHz, DMSO) δ 12.02 (s, 1H), 10.21 (s, 1H), 8.93 (d, J=5 Hz, 1H), 8.74 (d, J=5 Hz, 1H), 8.55 (s, 1H), 8.33 (s, 1H), 7.72 (d, J=5 Hz, 1H), 7.57 (s, 1H), 7.12-7.04 (m, 3H), 6.53 (d, J=10 Hz, 2H), 5.72 (s, 1H), 4.46-4.39 (m, 1H), 3.66 (t, J=5 Hz, 2H), 3.46-3.44 (m, 1H), 3.37-3.34 (m, 2H), 3.26-3.19 (m, 1H), 3.07-3.03 (m, 1H), 2.96-2.85 (m, 1H), 2.68 (t, J=5 Hz, 2H), 2.65-2.52 (m, 5H), 2.48-2.35 (m, 3H), 2.22-2.12 (m, 3H), 2.02-1.90 (m, 4H), 1.86-1.77 (m, 1H), 1.63 (s, 6H), 1.54-1.43 (m, 2H). [M+H]⁺=784.7

### Example 311: 3-cyano-N-(2-((1S,4r)-4-((S)-4-(1-(4-((R)-2,6-dioxopiperidin-3-yl)-3,5-difluorophenyl)azetidine-3-carbonyl)-3-methylpiperazin-1-yl)cyclohexyl)-6-(2-hydroxypropan-2-yl)-2H-indazol-5-yl)pyrrolo[1,2-b]pyridazine-7-carboxamide

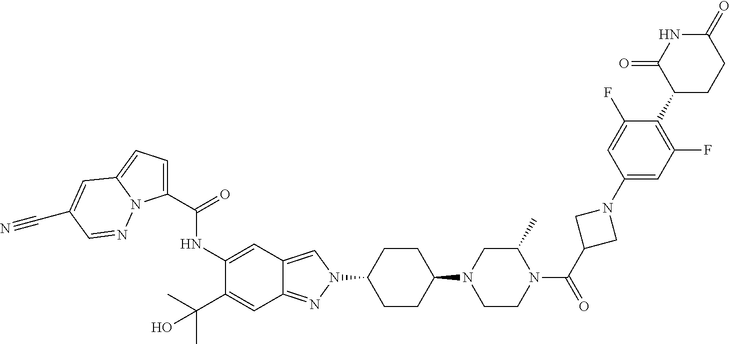 [SMILES: C[C@H]1CN([C@H]2CC[C@H](n3cc4cc(NC(=O)c5ccc6cc(C#N)cnn56)c(C(C)(C)O)cc4n3)CC2)CCN1C(=O)C1CN(c2cc(F)c([C@H]3CCC(=O)NC3=O)c(F)c2)C1]

The title compound (45.86 mg, 54.1%) was prepared in a manner similar to that described in Example 178. ¹H NMR (500 MHz, DMSO) δ 12.02 (s, 1H), 10.86 (s, 1H), 8.93 (d, J=5 Hz, 1H), 8.74 (d, J=5 Hz, 1H), 8.55 (s, 1H), 8.33 (s, 1H), 7.72 (d, J=5 Hz, 1H), 7.57 (s, 1H), 7.09 (d, J=5 Hz, 1H), 6.17 (dd, J=10, 5 Hz, 2H), 5.72 (s, 1H), 4.57-4.51 (m, 0.5H), 4.46-4.39 (m, 1H), 4.24-4.17 (m, 0.5H), 4.10-3.96 (m, 3H), 3.96-3.76 (m, 3.5H), 3.42-3.36 (m, 0.5H), 3.22-3.15 (m, 0.5H), 2.88-2.70 (m, 3.5H), 2.48-2.42 (m, 2H), 2.39-2.28 (m, 1H), 2.26-2.14 (m, 3H), 2.13-2.03 (m, 1H), 2.00-1.87 (m, 5H), 1.63 (s, 6H), 1.55-1.40 (m, 2H), 1.28-1.25 (m, 1.5H), 1.17-1.14 (m, 1.5H). [M+H]⁺=847.4

### Example 312: 3-cyano-N-(2-((1S,4r)-4-((S)-4-(1-(4-(2,4-dioxotetrahydropyrimidin-1(2H)-yl)-3,5-difluorophenyl)azetidine-3-carbonyl)-3-methylpiperazin-1-yl)cyclohexyl)-6-(2-hydroxypropan-2-yl)-2H-indazol-5-yl)pyrrolo[1,2-b]pyridazine-7-carboxamide

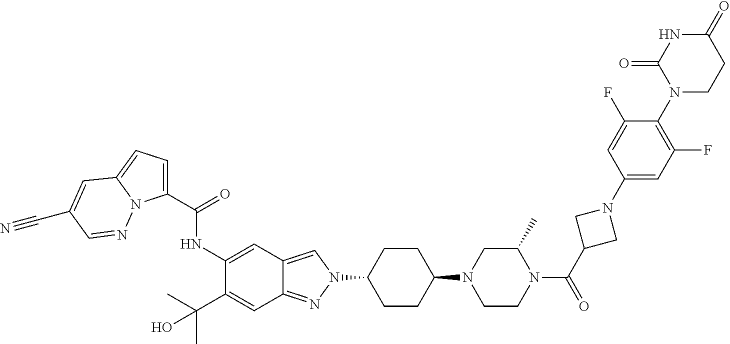 [SMILES: C[C@H]1CN([C@H]2CC[C@H](n3cc4cc(NC(=O)c5ccc6cc(C#N)cnn56)c(C(C)(C)O)cc4n3)CC2)CCN1C(=O)C1CN(c2cc(F)c(N3CCC(=O)NC3=O)c(F)c2)C1]

The title compound (39.48 mg, 46.6%) was prepared in a manner similar to that described in Example 311. ¹H NMR (500 MHz, DMSO) δ 12.02 (s, 1H), 10.49 (s, 1H), 8.93 (d, J=5 Hz, 1H), 8.74 (d, J=5 Hz, 1H), 8.55 (s, 1H), 8.33 (s, 1H), 7.72 (d, J=5 Hz, 1H), 7.57 (s, 1H), 7.09 (d, J=5 Hz, 1H), 6.25 (dd, J=10, 5 Hz, 2H), 5.72 (s, 1H), 4.57-4.51 (m, 0.5H), 4.46-4.39 (m, 1H), 4.24-4.17 (m, 0.5H), 4.13-3.78 (m, 5.5H), 3.59 (t, J=5 Hz, 2H), 3.42-3.36 (m, 0.5H), 3.22-3.15 (m, 0.5H), 2.88-2.81 (m, 1.5H), 2.75-2.71 (m, 1H), 2.70-2.65 (m, 2H), 2.48-2.43 (m, 1H), 2.40-2.27 (m, 1H), 2.26-2.13 (m, 3H), 2.00-1.87 (m, 4H), 1.63 (s, 6H), 1.55-1.41 (m, 2H), 1.28-1.25 (m, 1.5H), 1.17-1.14 (m, 1.5H). [M+H]⁺=848.4.

### Example 201: 3-cyano-N-(2-((1R,3r)-3-(4-(1-(4-((R)-2,6-dioxopiperidin-3-yl)-3,5-difluorophenyl)azetidine-3-carbonyl)piperazin-1-yl)cyclobutyl)-6-(2-hydroxypropan-2-yl)-2H-indazol-5-yl)pyrrolo[1,2-b]pyridazine-7-carboxamide

### Step 1: tert-butyl 4-(3-(((benzyloxy)carbonyl)amino)cyclobutyl)piperazine-1-carboxylate

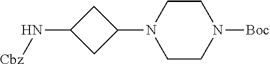 [SMILES: CC(C)(C)OC(=O)N1CCN(C2CC(NC(=O)OCc3ccccc3)C2)CC1]

To a solution of methyl benzyl (3-oxocyclobutyl)carbamate (10 g, 45.6 mmol) in DCE (200 mL) was added tert-butyl piperazine-1-carboxylate (8.5 g, 45.6 mmol) and NaBH(OAc)₃ (14.5 g, 68.4 mmol). The mixture was stirred for 16 hrs at 50° C. under N₂. The mixture was cooled to rt and concentrated in vacuo. The residue was purified by silica gel column chromatography (PE:EA=3:1) to give product (14.38 g, 81%). [M+H]⁺=390.2.

### Step 2: tert-butyl 4-(3-aminocyclobutyl)piperazine-1-carboxylate

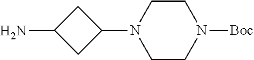 [SMILES: CC(C)(C)OC(=O)N1CCN(C2CC(N)C2)CC1]

To a solution of tert-butyl 4-(3-(((benzyloxy)carbonyl)amino)cyclobutyl)piperazine-1-carboxylate (14.38 g, 36.94 mmol) in MeOH (200 mL) was added Pd/C (10%, wet, 50% water). The mixture was stirred for 14 hrs at rt under H₂ atmosphere. The suspension was filtered and the filtrate was concentrated to give product (9 g, 95%). [M+H]⁺=256.2.

### Step 3: methyl 2-((1r,3r)-3-(4-(tert-butoxycarbonyl)piperazin-1-yl)cyclobutyl)-2H-indazole-6-carboxylate

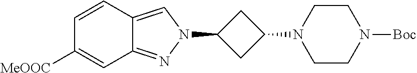 [SMILES: COC(=O)c1ccc2cn([C@H]3C[C@H](N4CCN(C(=O)OC(C)(C)C)CC4)C3)nc2c1]

To a solution of methyl 4-formyl-3-nitrobenzoate (7.37 g, 35.23 mmol) in propan-2-ol (100 mL) was added tert-butyl 4-(3-aminocyclobutyl)piperazine-1-carboxylate (9 g, 35.23 mmol). The mixture was stirred for 3 hrs at 80° C. under N₂. To the above solution was added tributylphosphane (21.35 g, 105.69 mmol). The mixture was stirred overnight at 80° C. under N₂. The mixture was cooled to rt and concentrated in vacuo. The residue was purified by silica gel column chromatography (PE:EA=3:1 to 1:1) to give product (5.82 g, 40%). [M+H]⁺=415.2.

### Step 4: methyl 5-nitro-2-((1r,3r)-3-(piperazin-1-yl)cyclobutyl)-2H-indazole-6-carboxylate

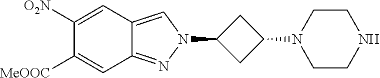 [SMILES: COC(=O)c1cc2nn([C@H]3C[C@H](N4CCNCC4)C3)cc2cc1[N+](=O)[O-]]

To a solution of methyl 2-((1r,3r)-3-(4-(tert-butoxycarbonyl)piperazin-1-yl)cyclobutyl)-2H-indazole-6-carboxylate (5.8 g, 14 mmol) in conc.H₂SO₄ (30 mL) was slowed added NaNO₃ (1.43 g, 16.8 mmol) at 0° C. The mixture was stirred for 2 hrs at 0° C. The mixture was quenched with ice-water, then adjusted to pH 9 with sat.Na₂CO₃ solution and extracted with DCM. The combined organic layers were dried over anhydrous Na₂SO₄, filtered and evaporated in vacuum to afford product (4.0 g, 80%), which was used without further purification. [M+H]⁺=360.2.

### Step 5: methyl 2-((1r,3r)-3-(4-(tert-butoxycarbonyl)piperazin-1-yl)cyclobutyl)-5-nitro-2H-indazole-6-carboxylate

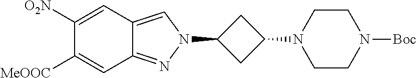 [SMILES: COC(=O)c1cc2nn([C@H]3C[C@H](N4CCN(C(=O)OC(C)(C)C)CC4)C3)cc2cc1[N+](=O)[O-]]

To a stirred solution of methyl 5-nitro-2-((1r,3r)-3-(piperazin-1-yl)cyclobutyl)-2H-indazole-6-carboxylate (4.0 g, 11.14 mmol) in DCM (60 mL) was added Et₃N (2.25 g, 22.28 mmol) and Boc₂O (2.91 g, 13.37 mmol). The reaction mixture was stirred overnight at rt. The mixture was concentrated in vacuo. The residue was purified by silica gel column chromatography (PE:EA=3:1 to 1:1) to give product (4.35 g, 85%). [M+H]⁺=460.2.

### Step 6: methyl 5-amino-2-((1r,3r)-3-(4-(tert-butoxycarbonyl)piperazin-1-yl)cyclobutyl)-2H-indazole-6-carboxylate

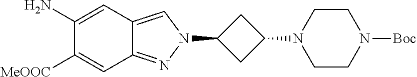 [SMILES: COC(=O)c1cc2nn([C@H]3C[C@H](N4CCN(C(=O)OC(C)(C)C)CC4)C3)cc2cc1N]

To a solution of methyl 2-((1r,3r)-3-(4-(tert-butoxycarbonyl)piperazin-1-yl)cyclobutyl)-5-nitro-2H-indazole-6-carboxylate (4.35 g, 9.47 mmol) in MeOH (50 mL) and water (10 mL) was added NH₄Cl (5.07 g, 94.7 mmol) and Fe powder (2.65 g, 47.4 mmol) at room temperature. The resulting mixture was stirred for 2 hrs at 70° C. The mixture was cooled to rt and concentrated in vacuo. The residue was purified by silica gel column chromatography (DCM:MeOH=15:1) to give product (3.25 g, 80%). [M+H]⁺=430.2.

### Step 7: methyl 2-((1r,3r)-3-(4-(tert-butoxycarbonyl)piperazin-1-yl)cyclobutyl)-5-(2,2,2-trifluoroacetamido)-2H-indazole-6-carboxylate

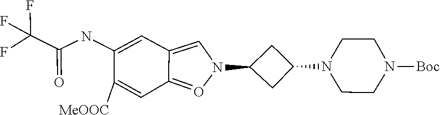

To a solution of methyl 5-amino-2-((1r,3r)-3-(4-(tert-butoxycarbonyl)piperazin-1-yl)cyclobutyl)-2H-indazole-6-carboxylate (3.25 g, 7.58 mmol) in DCM (30 mL) was added TFAA (1.91 g, 9.10 mmol) at 0° C. The resulting mixture was stirred for 2 hrs at 0° C. The mixture was quenched with water (20 mL) and concentrated in vacuo. The residue was purified by silica gel column chromatography (EA) to give product (3.60 g, 90.7%). [M+H]⁺=526.2.

### Step 8: tert-butyl 4-((1r,3r)-3-(6-(2-hydroxypropan-2-yl)-5-(2,2,2-trifluoroacetamido)-2H-indazol-2-yl)cyclobutyl)piperazine-1-carboxylate

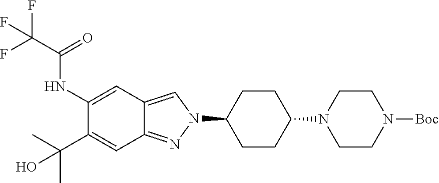 [SMILES: CC(C)(C)OC(=O)N1CCN([C@H]2CC[C@H](n3cc4cc(NC(=O)C(F)(F)F)c(C(C)(C)O)cc4n3)CC2)CC1]

To a solution of methyl 2-((1r,3r)-3-(4-(tert-butoxycarbonyl)piperazin-1-yl)cyclobutyl)-5-(2,2,2-trifluoroacetamido)-2H-indazole-6-carboxylate (3.60 g, 6.84 mmol) in THF (30 mL) was added MeMgBr (3M in THF) (22.8 mL, 68.4 mmol) at 0° C. The resulting mixture was stirred for 6 hours at rt. The mixture was cooled to 0° C. and quenched with saturated NH₄Cl solution. The mixture was extracted with DCM (50 mL×3) and concentrated. The residue was purified by silica gel column chromatography (DCM:MeOH=15:1) to give product (3.06 g, 85%). [M+H]⁺=526.2.

### Step 9: tert-butyl 4-((1r,3r)-3-(5-amino-6-(2-hydroxypropan-2-yl)-2H-indazol-2-yl)cyclobutyl)piperazine-1-carboxylate

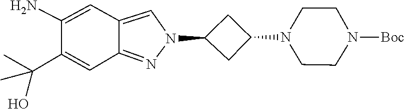 [SMILES: CC(C)(C)OC(=O)N1CCN([C@H]2C[C@H](n3cc4cc(N)c(C(C)(C)O)cc4n3)C2)CC1]

To a solution of methyl tert-butyl 4-((1r,3r)-3-(6-(2-hydroxypropan-2-yl)-5-(2,2,2-trifluoroacetamido)-2H-indazol-2-yl)cyclobutyl)piperazine-1-carboxylate (3.06 g, 5.81 mmol) in MeOH (30 mL) was added NaOH (2.32 g, 58.1 mmol) in water (20 mL) at rt. The resulting mixture was stirred for 2 hrs at 70° C. The mixture was extracted with DCM. The combined organic layers were dried over anhydrous Na₂SO₄, filtered and evaporated in vacuum. The residue was purified by silica gel column chromatography (DCM:MeOH=10:1) to give product (2.04 g, 82%). [M+H]⁺=430.3.

### Step 10: tert-butyl 4-((1r,3r)-3-(5-(3-cyanopyrrolo[1,2-b]pyridazine-7-carboxamido)-6-(2-hydroxypropan-2-yl)-2H-indazol-2-yl)cyclobutyl)piperazine-1-carboxylate

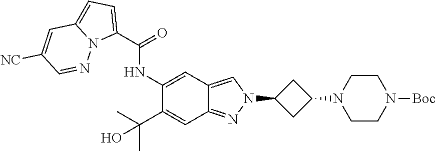 [SMILES: CC(C)(C)OC(=O)N1CCN([C@H]2C[C@H](n3cc4cc(NC(=O)c5ccc6cc(C#N)cnn56)c(C(C)(C)O)cc4n3)C2)CC1]

To a solution of tert-butyl 4-((1r,3r)-3-(5-amino-6-(2-hydroxypropan-2-yl)-2H-indazol-2-yl)cyclobutyl)piperazine-1-carboxylate (2.04 g, 4.76 mmol) and 3-cyanopyrrolo[1,2-b]pyridazine-7-carboxylic acid (0.89 g, 4.76 mmol) in DMF (10 mL) was added TEA (0.96 g, 9.53 mmol) and HATU (2.17 g, 5.71 mmol) at rt. The resulting mixture was stirred for 2 hrs at rt. The mixture was diluted with water and extracted with DCM. The combined organic layers were dried over anhydrous Na₂SO₄, filtered and evaporated in vacuum. The residue was purified by silica gel column chromatography (DCM:MeOH=20:1) to give product (2.42 g, 85%). [M+H]⁺=599.3.

### Step 11: 3-cyano-N-(6-(2-hydroxypropan-2-yl)-2-((1r,3r)-3-(piperazin-1-yl)cyclobutyl)-2H-indazol-5-yl)pyrrolo[1,2-b]pyridazine-7-carboxamide

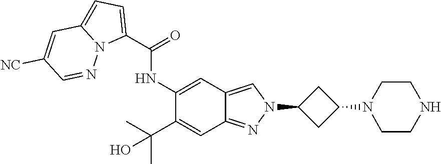 [SMILES: CC(C)(O)c1cc2nn([C@H]3C[C@H](N4CCNCC4)C3)cc2cc1NC(=O)c1ccc2cc(C#N)cnn12]

To a solution of tert-butyl 4-((1r,3r)-3-(5-(3-cyanopyrrolo[1,2-b]pyridazine-7-carboxamido)-6-(2-hydroxypropan-2-yl)-2H-indazol-2-yl)cyclobutyl)piperazine-1-carboxylate (2.42 g, 4.11 mmol) in MeOH (10 mL) was added DCM (10 mL) and con. HCl (10 mL). The resulting mixture was stirred for 2 hrs at rt. The mixture was quenched with sat. Na₂CO₃ solution and extracted with DCM. The combined organic layers were dried over anhydrous Na₂SO₄, filtered and evaporated in vacuum to give product (2.01 g, 84%). [M+H]⁺=499.3.

### Step 12: 3-cyano-N-(2-((1R,3r)-3-(4-(1-(4-((R)-2,6-dioxopiperidin-3-yl)-3,5-difluorophenyl)azetidine-3-carbonyl)piperazin-1-yl)cyclobutyl)-6-(2-hydroxypropan-2-yl)-2H-indazol-5-yl)pyrrolo[1,2-b]pyridazine-7-carboxamide

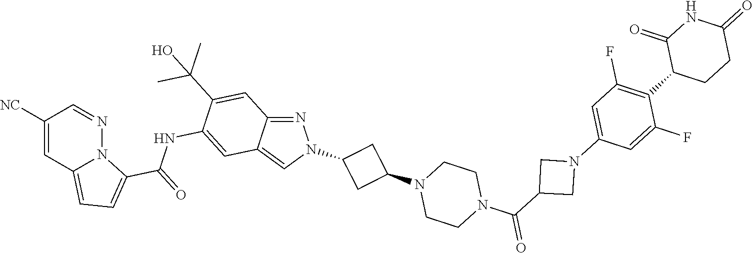 [SMILES: CC(C)(O)c1cc2nn([C@H]3C[C@H](N4CCN(C(=O)C5CN(c6cc(F)c([C@H]7CCC(=O)NC7=O)c(F)c6)C5)CC4)C3)cc2cc1NC(=O)c1ccc2cc(C#N)cnn12]

To a solution of 3-cyano-N-(6-(2-hydroxypropan-2-yl)-2-((1r,3r)-3-(piperazin-1-yl)cyclobutyl)-2H-indazol-5-yl)pyrrolo[1,2-b]pyridazine-7-carboxamide (40 mg, 0.08 mmol) and (R)-1-(4-(2,6-dioxopiperidin-3-yl)-3,5-difluorophenyl)azetidine-3-carboxylic acid (26 mg, 0.08 mmol) in DCM (5 mL) was added DIPEA (30.96 mg, 0.24 mmol) and T₃P in EtOAc (50%, 101.76 mg, 0.16 mmol) at rt. The resulting mixture was stirred for 2 hrs at rt. The mixture was diluted with water and extracted with DCM. The combined organic layers were dried over anhydrous Na₂SO₄, filtered and evaporated in vacuum. The residue was purified by Prep-HPLC (C-18 column chromatography (0.1% FA in water:acetonitrile=90:10˜60:40 gradient elution) to give the desired product (30 mg, 46.8%). [M+H]⁺=805.3. ¹H NMR (500 MHz, DMSO) δ 12.04 (s, 1H), 10.86 (s, 1H), 8.93 (d, J=2.2 Hz, 1H), 8.74 (d, J=2.2 Hz, 1H), 8.57 (s, 1H), 8.41 (s, 1H), 7.72 (d, J=4.8 Hz, 1H), 7.62 (s, 1H), 7.09 (d, J=4.8 Hz, 1H), 6.17 (d, J=11.0 Hz, 2H), 5.75 (s, 1H), 5.21-5.12 (m, 1H), 4.04 (t, J=7.4 Hz, 3H), 3.93 (t, J=6.0 Hz, 2H), 3.85 (dt, J=16.2, 8.0 Hz, 1H), 3.54 (brs, 2H), 3.43-3.34 (m, 3H), 3.21-3.12 (m, 1H), 2.83-2.72 (m, 1H), 2.67-2.54 (m, 4H), 2.41-2.28 (m, 4H), 2.13-2.01 (m, 1H), 1.95 (dd, J=9.3, 3.8 Hz, 1H), 1.64 (s, 6H).

### Example 329: 3-cyano-N-(2-((1R,3r)-3-(4-((R)-1-(4-((R)-2,6-dioxopiperidin-3-yl)-3,5-difluorophenyl)pyrrolidine-3-carbonyl)piperazin-1-yl)cyclobutyl)-6-(2-hydroxypropan-2-yl)-2H-indazol-5-yl)pyrrolo[1,2-b]pyridazine-7-carboxamide

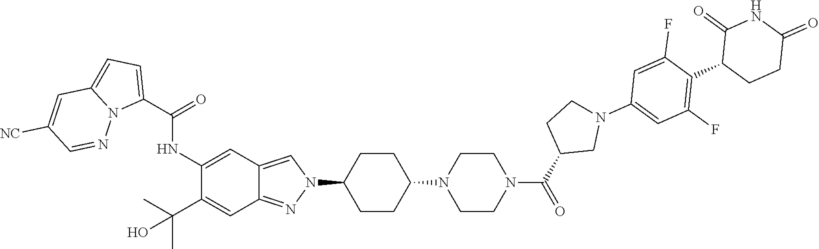 [SMILES: CC(C)(O)c1cc2nn([C@H]3CC[C@H](N4CCN(C(=O)[C@@H]5CCN(c6cc(F)c([C@H]7CCC(=O)NC7=O)c(F)c6)C5)CC4)CC3)cc2cc1NC(=O)c1ccc2cc(C#N)cnn12]

The title compound (30 mg, 49%) was prepared in a manner similar to that described in Example 201.

¹H NMR (500 MHz, DMSO) δ 12.05 (s, 1H), 10.84 (s, 1H), 8.93 (d, J=2.1 Hz, 1H), 8.74 (d, J=2.1 Hz, 1H), 8.57 (s, 1H), 8.41 (s, 1H), 7.72 (d, J=4.8 Hz, 1H), 7.62 (s, 1H), 7.09 (d, J=4.8 Hz, 1H), 6.23 (d, J=12.2 Hz, 2H), 5.76 (s, 1H), 5.20-5.13 (m, 1H), 4.02 (dd, J=12.5, 4.9 Hz, 1H), 3.67-3.50 (m, 5H), 3.49-3.40 (m, 1H), 3.32-3.21 (m, 4H), 3.19-3.13 (m, 1H), 2.84-2.73 (m, 1H), 2.66-2.55 (m, 4H), 2.46-2.27 (m, 4H), 2.23-2.04 (m, 3H), 2.01-1.89 (m, 1H), 1.64 (s, 6H). [M+H]⁺=819.4.

### Example 330: 3-cyano-N-(2-((1R,3r)-3-(4-(1-(4-((R)-2,6-dioxopiperidin-3-yl)-3,5-difluorophenyl)piperidin-4-yl)piperazin-1-yl)cyclobutyl)-6-(2-hydroxypropan-2-yl)-2H-indazol-5-yl)pyrrolo[1,2-b]pyridazine-7-carboxamide

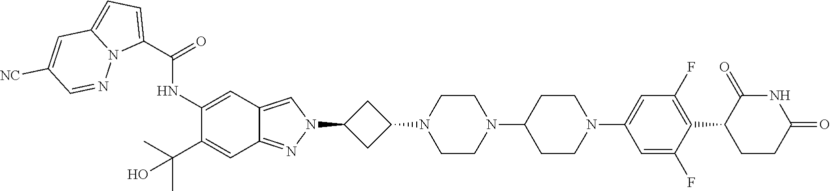 [SMILES: CC(C)(O)c1cc2nn([C@H]3C[C@H](N4CCN(C5CCN(c6cc(F)c([C@H]7CCC(=O)NC7=O)c(F)c6)CC5)CC4)C3)cc2cc1NC(=O)c1ccc2cc(C#N)cnn12]

To a solution of 3-cyano-N-(6-(2-hydroxypropan-2-yl)-2-((1r,3r)-3-(piperazin-1-yl)cyclobutyl)-2H-indazol-5-yl)pyrrolo[1,2-b]pyridazine-7-carboxamide (40 mg, 0.08 mmol) and (R)-3-(2,6-difluoro-4-(4-oxopiperidin-1-yl)phenyl)piperidine-2,6-dione (25.86 mg, 0.08 mmol) in DCE (5 mL) was added NaBH(OAc)3 (34 mg, 0.16 mmol) at rt. The resulting mixture was stirred for 2 hrs at 55° C. The mixture was diluted with water and extracted with DCM. The combined organic layers were dried over anhydrous Na₂SO₄, filtered and evaporated in vacuum. The residue was purified by Prep-HPLC (C-18 column chromatography (0.1% FA in water:acetonitrile=90:10˜60:40 gradient elution) to give the desired product (20 mg, 31.0%). [M+H]⁺=805.4 ¹H NMR (500 MHz, DMSO) δ 12.04 (s, 1H), 10.87 (s, 1H), 8.93 (d, J=2.2 Hz, 1H), 8.74 (d, J=2.2 Hz, 1H), 8.56 (s, 1H), 8.39 (s, 1H), 7.72 (d, J=4.8 Hz, 1H), 7.62 (s, 1H), 7.09 (d, J=4.8 Hz, 1H), 6.63 (d, J=12.8 Hz, 2H), 5.75 (s, 1H), 5.18-5.10 (m, 1H), 4.04 (dd, J=12.6, 5.0 Hz, 1H), 3.79 (d, J=12.3 Hz, 2H), 3.29 (d, J=13.6 Hz, 2H), 3.09 (s, 1H), 2.84-2.70 (m, 3H), 2.67-2.50 (m, 9H), 2.42-2.25 (m, 3H), 2.11-2.05 (m, 1H), 1.99-1.92 (m, 1H), 1.83 (d, J=10.9 Hz, 2H), 1.64 (s, 6H), 1.51-1.39 (m, 2H).

### Example 331: 3-cyano-N-(2-((1R,3r)-3-(4-(1-(4-((R)-2,6-dioxopiperidin-3-yl)-3,5-difluorophenyl)azetidin-3-yl)piperazin-1-yl)cyclobutyl)-6-(2-hydroxypropan-2-yl)-2H-indazol-5-yl)pyrrolo[1,2-b]pyridazine-7-carboxamide

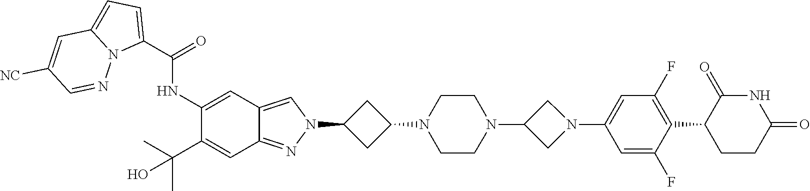 [SMILES: CC(C)(O)c1cc2nn([C@H]3C[C@H](N4CCN(C5CN(c6cc(F)c([C@H]7CCC(=O)NC7=O)c(F)c6)C5)CC4)C3)cc2cc1NC(=O)c1ccc2cc(C#N)cnn12]

The title compound (21 mg, 34%) was prepared in a manner similar to that described in Example 330. ¹H NMR (500 MHz, DMSO) δ 12.04 (s, 1H), 10.86 (s, 1H), 8.93 (d, J=2.1 Hz, 1H), 8.74 (d, J=2.0 Hz, 1H), 8.56 (s, 1H), 8.40 (s, 1H), 7.72 (d, J=4.8 Hz, 1H), 7.62 (s, 1H), 7.09 (d, J=4.8 Hz, 1H), 6.12 (d, J=11.1 Hz, 2H), 5.75 (s, 1H), 5.13 (t, J=9.2 Hz, 1H), 4.03 (dd, J=12.6, 4.9 Hz, 1H), 3.92 (t, J=7.1 Hz, 2H), 3.65 (s, 2H), 3.31-3.22 (m, 3H), 3.17-3.08 (m, 1H), 2.85-2.72 (m, 1H), 2.65-2.52 (m, 6H), 2.46-2.25 (m, 5H), 2.08 (dt, J=16.4, 11.4 Hz, 1H), 1.94 (dd, J=14.1, 8.9 Hz, 1H), 1.64 (s, 6H). [M+H]⁺=777.30.

### Example 309: 3-cyano-N-(2-((1R,4r)-4-((R)-4-(1-(4-((R)-2,6-dioxopiperidin-3-yl)-3,5-difluorophenyl)azetidine-3-carbonyl)-3-methylpiperazin-1-yl)cyclohexyl)-6-(2-hydroxypropan-2-yl)-2H-indazol-5-yl)pyrrolo[1,2-b]pyridazine-7-carboxamide

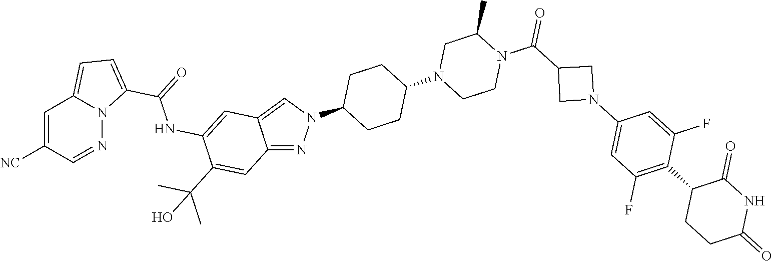 [SMILES: C[C@@H]1CN([C@H]2CC[C@H](n3cc4cc(NC(=O)c5ccc6cc(C#N)cnn56)c(C(C)(C)O)cc4n3)CC2)CCN1C(=O)C1CN(c2cc(F)c([C@H]3CCC(=O)NC3=O)c(F)c2)C1]

The title compound was prepared in a manner similar to that described in Example 178. ¹H NMR (500 MHz, DMSO) δ 12.03 (s, 1H), 12.03 (s, 1H), 10.86 (s, 1H), 10.86 (s, 1H), 8.93 (d, J=2.0 Hz, 1H), 8.74 (d, J=2.0 Hz, 1H), 8.55 (s, 1H), 8.33 (s, 1H), 7.72 (d, J=4.8 Hz, 1H), 7.57 (s, 1H), 7.09 (d, J=4.8 Hz, 1H), 6.17 (dd, J=11.0, 7.2 Hz, 2H), 5.73 (s, 1H), 4.57-4.51 (m, 1H), 4.43 (t, J=11.5 Hz, 1H), 4.20 (d, J=12.9 Hz, 0.5H), 4.05 (m, 3H), 3.97-3.74 (m, 3.5H), 3.43-3.36 (m, 1.5H), 3.24-3.13 (m, 0.5H), 2.90-2.77 (m, 2H), 2.76-2.63 (m, 1H), 2.48-2.42 (m, 1H), 2.34 (dd, J=31.5, 8.0 Hz, 1H), 2.28-2.02 (m, 4H), 2.03-1.87 (m, 5H), 1.61 (s, 6H), 1.56-1.39 (m, 2H), 1.24 (d, 12.1 Hz, 1.2H), 1.11 (d, J=12.1 Hz, 2H). [M+H]⁺=847.4

### Example 310: 3-cyano-N-(2-((1R,4r)-4-((R)-4-((1-(4-((R)-2,6-dioxopiperidin-3-yl)-3,5-difluorophenyl)azetidin-3-yl)methyl)-3-methylpiperazin-1-yl)cyclohexyl)-6-(2-hydroxypropan-2-yl)-2H-indazol-5-yl)pyrrolo[1,2-b]pyridazine-7-carboxamide

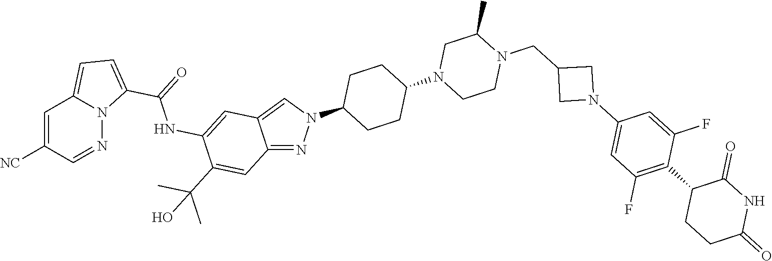 [SMILES: C[C@@H]1CN([C@H]2CC[C@H](n3cc4cc(NC(=O)c5ccc6cc(C#N)cnn56)c(C(C)(C)O)cc4n3)CC2)CCN1CC1CN(c2cc(F)c([C@H]3CCC(=O)NC3=O)c(F)c2)C1]

To a solution of 3-cyano-N-(6-(2-hydroxypropan-2-yl)-2-((1R,4r)-4-((R)-3-methylpiperazin-1-yl)cyclohexyl)-2H-indazol-5-yl)pyrrolo[1,2-b]pyridazine-7-carboxamide (40 mg, 0.074 mmol) and (R)-1-(4-(2,6-dioxopiperidin-3-yl)-3,5-difluorophenyl)azetidine-3-carbaldehyde (22.81 mg, 0.074 mmol) in DCE (5 mL) was added NaBH(OAc)₃ (32 mg, 0.148 mmol) at rt. The resulting mixture was stirred for 12 hrs at rt. The mixture was diluted with water and extracted with DCM. The combined organic layers were dried over anhydrous Na₂SO₄, filtered and evaporated in vacuum. The residue was purified by Prep-HPLC (C-18 column chromatography (0.1% FA in water:acetonitrile=90:10˜60:40 gradient elution) to give the desired product (32 mg, 52.0%). ¹H NMR (500 MHz, DMSO) δ 12.02 (s, 1H), 10.85 (s, 1H), 8.93 (d, J=1.9 Hz, 1H), 8.74 (d, J=1.8 Hz, 1H), 8.55 (s, 1H), 8.33 (s, 1H), 8.15 (s, 0.53H, FA), 7.72 (d, J=4.8 Hz, 1H), 7.57 (s, 1H), 7.09 (d, J=4.8 Hz, 1H), 6.10 (d, J=11.2 Hz, 2H), 5.73 (s, 1H), 4.42 (t, J=11.3 Hz, 1H), 4.02 (dd, J=12.5, 4.9 Hz, 1H), 3.98-3.86 (m, 2H), 3.54-3.49 (m, 2H), 2.98-2.86 (m, 3H), 2.84-2.69 (m, 4H), 2.44-2.31 (m, 4H), 2.21-2.15 (m, 3H), 2.13-2.04 (m, 2H), 1.99-1.87 (m, 5H), 1.63 (s, 6H), 1.47 (d, J=11.9 Hz, 2H), 1.01 (d, J=6.0 Hz, 3H). [M+H]⁺=833.407.

### Example 336: 3-cyano-N-(2-((1r,4r)-4-(4-(1-(4-((R)-2,6-dioxopiperidin-3-yl)-3,5-difluorophenyl)azetidine-3-carbonyl)piperazin-1-yl)cyclohexyl)-6-methoxy-2H-indazol-5-yl)pyrrolo[1,2-b]pyridazine-7-carboxamide

### Step 1: tert-butyl 4-((1r,4r)-4-(5-(3-cyanopyrrolo[1,2-b]pyridazine-7-carboxamido)-6-methoxy-2H-indazol-2-yl)cyclohexyl)piperazine-1-carboxylate

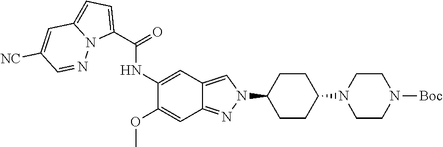 [SMILES: COc1cc2nn([C@H]3CC[C@H](N4CCN(C(=O)OC(C)(C)C)CC4)CC3)cc2cc1NC(=O)c1ccc2cc(C#N)cnn12]

To a solution of tert-butyl 4-((1r,4r)-4-(5-amino-6-methoxy-2H-indazol-2-yl)cyclohexyl)piperazine-1-carboxylate (500 mg, 1.164 mmol) and 3-cyanopyrrolo[1,2-b]pyridazine-7-carboxylic acid (218 mg, 1.164 mmol) in DMF (5 mL) was added TEA (235 mg, 2.33 mmol) and HATU (530 mg, 1.39 mmol) at rt.

The resulting mixture was stirred for 2 hrs at rt. The mixture was diluted with water and extracted with DCM. The combined organic layers were dried over anhydrous Na₂SO₄, filtered and evaporated in vacuum. The residue was purified by silica gel column chromatography (DCM:MeOH=20:1) to give product (592 mg, 85%). [M+H]⁺=599.3.

### Step 2: 3-cyano-N-(6-methoxy-2-((1r,4r)-4-(piperazin-1-yl)cyclohexyl)-2H-indazol-5-yl)pyrrolo[1,2-b]pyridazine-7-carboxamide

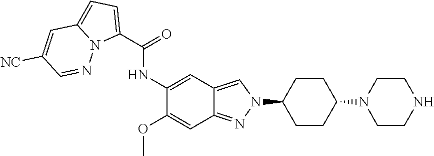 [SMILES: COc1cc2nn([C@H]3CC[C@H](N4CCNCC4)CC3)cc2cc1NC(=O)c1ccc2cc(C#N)cnn12]

To a solution of tert-butyl 4-((1r,4r)-4-(5-(3-cyanopyrrolo[1,2-b]pyridazine-7-carboxamido)-6-methoxy-2H-indazol-2-yl)cyclohexyl)piperazine-1-carboxylate (590 mg, 0.986 mmol) in MeOH (10 mL) was added DCM (10 mL) and con. HCl (10 mL). The resulting mixture was stirred for 2 hrs at rt. The mixture was quenched with sat.Na₂CO₃ solution and extracted with DCM. The combined organic layers were dried over anhydrous Na₂SO₄, filtered and evaporated in vacuum to give product (420 mg, 86%). [M+H]+=499.3.

### Step 3: 3-cyano-N-(2-((1r,4r)-4-(4-(1-(4-((R)-2,6-dioxopiperidin-3-yl)-3,5-difluorophenyl)azetidine-3-carbonyl)piperazin-1-yl)cyclohexyl)-6-methoxy-2H-indazol-5-yl)pyrrolo[1,2-b]pyridazine-7-carboxamide

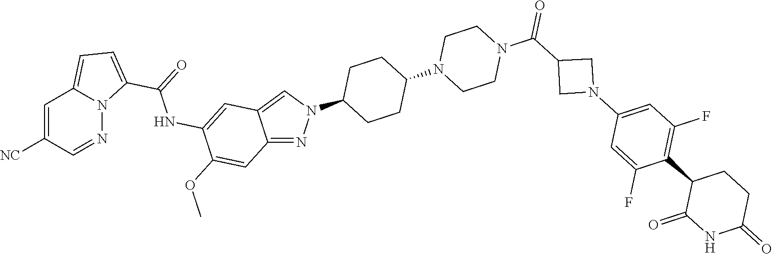 [SMILES: COc1cc2nn([C@H]3CC[C@H](N4CCN(C(=O)C5CN(c6cc(F)c([C@H]7CCC(=O)NC7=O)c(F)c6)C5)CC4)CC3)cc2cc1NC(=O)c1ccc2cc(C#N)cnn12]

To a solution of 3-cyano-N-(6-methoxy-2-((1r,4r)-4-(piperazin-1-yl)cyclohexyl)-2H-indazol-5-yl)pyrrolo[1,2-b]pyridazine-7-carboxamide (40 mg, 0.08 mmol) in DCM (5 mL) was added DIPEA (30.96 mg, 0.24 mmol) and T3P in EtOAc (50%, 101.76 mg, 0.16 mmol) at rt. The resulting mixture was stirred for 2 hrs at rt. The mixture was diluted with water and extracted with DCM. The combined organic layers were dried over anhydrous Na₂SO₄, filtered and evaporated in vacuum. The residue was purified by Prep-HPLC (C-18 column chromatography (0.1% FA in water:acetonitrile=90:10˜60:40 gradient elution) to give the desired product (30 mg, 46.8%). ¹H NMR (500 MHz, DMSO) δ 11.52 (s, 1H), 11.52 (s, 1H), 10.86 (s, 1H), 10.86 (s, 1H), 9.07 (d, J=1.7 Hz, 1H), 9.07 (d, J=1.7 Hz, 1H), 8.99 (d, J=1.7 Hz, 1H), 8.99 (d, J=1.7 Hz, 1H), 8.75 (s, 1H), 8.75 (s, 1H), 8.30 (s, 1H), 8.30 (s, 1H), 7.77 (d, J=4.8 Hz, 1H), 7.77 (d, J=4.8 Hz, 1H), 7.12 (d, J=5.1 Hz, 2H), 6.17 (d, J=11.1 Hz, 2H), 6.17 (d, J=11.1 Hz, 2H), 4.41-4.32 (m, 1H), 4.07 (s, 3H), 4.03 (t, J=8.0 Hz, 3H), 3.92 (t, J=6.4 Hz, 2H), 3.87-3.79 (m, 1H), 3.53-3.45 (m, 3H), 3.43-3.40 (m, 2H), 2.84-2.72 (m, 1H), 2.60-2.52 (m, 3H), 2.47-2.42 (m, 2H), 2.18 (d, J=9.3 Hz, 2H), 2.13-2.02 (m, 1H), 1.98-1.86 (m, 5H), 1.56-1.42 (m, 2H). [M+H]⁺=805.3.

### Example 337: 3-cyano-N-(2-((1R,4r)-4-(4-(4-((R)-2,6-dioxopiperidin-3-yl)-3,5-difluorophenethyl)piperazin-1-yl)cyclohexyl)-6-methoxy-2H-indazol-5-yl)pyrrolo[1,2-b]pyridazine-7-carboxamide

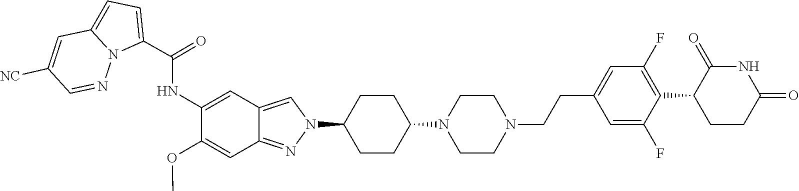 [SMILES: COc1cc2nn([C@H]3CC[C@H](N4CCN(CCc5cc(F)c([C@H]6CCC(=O)NC6=O)c(F)c5)CC4)CC3)cc2cc1NC(=O)c1ccc2cc(C#N)cnn12]

To a solution of 3-cyano-N-(6-methoxy-2-((1r,4r)-4-(piperazin-1-yl)cyclohexyl)-2H-indazol-5-yl)pyrrolo[1,2-b]pyridazine-7-carboxamide (40 mg, 0.08 mmol) and (R)-2-(4-(2,6-dioxopiperidin-3-yl)-3,5-difluorophenyl)acetaldehyde (21.5 mg, 0.08 mmol) in DCE (5 mL) was added NaBH(OAc)₃ (34 mg, 0.16 mmol) at rt. The resulting mixture was stirred for 12 hrs at rt. The mixture was diluted with water and extracted with DCM. The combined organic layers were dried over anhydrous Na₂SO₄, filtered and evaporated in vacuum. The residue was purified by Prep-HPLC (C-18 column chromatography (0.1% FA in water:acetonitrile=90:10˜60:40 gradient elution) to give the desired product (33 mg, 55.0%).

¹H NMR (500 MHz, DMSO) δ 11.52 (s, 1H), 10.95 (s, 1H), 9.08 (d, J=1.9 Hz, 1H), 8.99 (d, J=1.9 Hz, 1H), 8.75 (s, 1H), 8.29 (s, 1H), 7.77 (d, J=4.8 Hz, 1H), 7.12 (d, J=4.8 Hz, 2H), 7.03 (d, J=10.1 Hz, 2H), 4.35 (t, J=11.3 Hz, 1H), 4.20 (dd, J=12.6, 4.9 Hz, 1H), 4.07 (s, 3H), 2.88-2.73 (m, 4H), 2.63-2.52 (m, 6H), 2.47-2.34 (m, 4H), 2.22-2.06 (m, 4H), 2.06-1.86 (m, 6H), 1.52-1.41 (m, 2H). [M+H]+=750.30.

### Example 338: 3-cyano-N-(2-((1r,4r)-4-(4-(1-(4-((R)-2,6-dioxopiperidin-3-yl)-3,5-difluorophenyl)piperidin-4-yl)piperazin-1-yl)cyclohexyl)-6-methoxy-2H-indazol-5-yl)pyrrolo[1,2-b]pyridazine-7-carboxamide

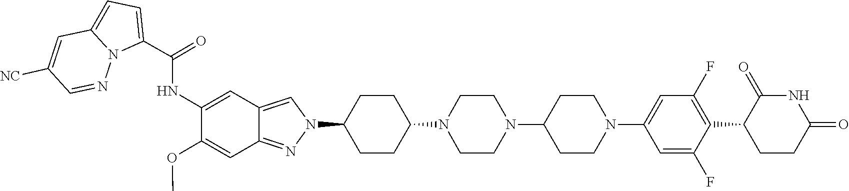 [SMILES: COc1cc2nn([C@H]3CC[C@H](N4CCN(C5CCN(c6cc(F)c([C@H]7CCC(=O)NC7=O)c(F)c6)CC5)CC4)CC3)cc2cc1NC(=O)c1ccc2cc(C#N)cnn12]

To a solution of 3-cyano-N-(6-methoxy-2-((1r,4r)-4-(piperazin-1-yl)cyclohexyl)-2H-indazol-5-yl)pyrrolo[1,2-b]pyridazine-7-carboxamide (40 mg, 0.08 mmol) and (R)-3-(2,6-difluoro-4-(4-oxopiperidin-1-yl)phenyl)piperidine-2,6-dione (25.9 mg, 0.08 mmol) in DCE (5 mL) was added NaBH(OAc)₃ (34 mg, 0.16 mmol) at rt. The resulting mixture was stirred for 12 hrs at 55° C. The mixture was diluted with water and extracted with DCM. The combined organic layers were dried over anhydrous Na₂SO₄, filtered and evaporated in vacuum. The residue was purified by Prep-HPLC (C-18 column chromatography (0.1% FA in water:acetonitrile=90:10˜60:40 gradient elution) to give the desired product (22 mg, 34.4%). ¹H NMR (500 MHz, DMSO) δ 11.52 (s, 1H), 10.85 (s, 1H), 9.08 (s, 1H), 8.99 (s, 1H), 8.75 (s, 1H), 8.29 (s, 1H), 7.77 (d, J=4.8 Hz, 1H), 7.12 (d, J=4.8 Hz, 2H), 6.10 (d, J=11.2 Hz, 2H), 4.38-4.31 (m, 1H), 4.07 (s, 3H), 4.02 (dd, J=12.4, 4.9 Hz, 1H), 3.92 (t, J=7.5 Hz, 2H), 3.52-3.43 (m, 4H), 2.94-2.87 (m, 1H), 2.82-2.71 (m, 1H), 2.60-2.52 (m, 5H), 2.45-2.31 (s, 5H), 2.16 (d, J=11.3 Hz, 2H), 2.07 (dd, J=24.1, 11.1 Hz, 1H), 1.97-1.85 (m, 5H), 1.51-1.40 (dd, J=23.4, 11.3 Hz, 2H). [M+H]⁺=805.40.

### Example 343: 2-((1r,4r)-4-(4-(1-(4-((R)-2,6-dioxopiperidin-3-yl)-3,5-difluorophenyl)azetidine-3-carbonyl)piperazin-1-yl)cyclohexyl)-N-(imidazo[1,2-b]pyridazin-3-yl)-6-methoxy-2H-indazole-5-carboxamide

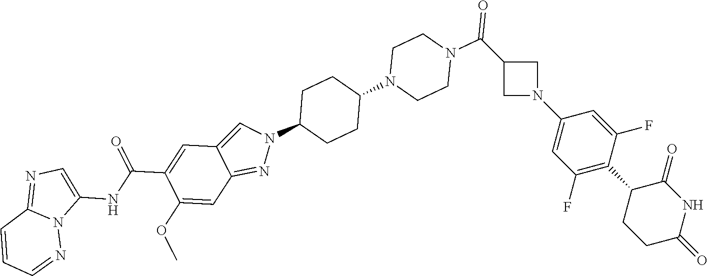 [SMILES: COc1cc2nn([C@H]3CC[C@H](N4CCN(C(=O)C5CN(c6cc(F)c([C@H]7CCC(=O)NC7=O)c(F)c6)C5)CC4)CC3)cc2cc1C(=O)Nc1cnc2cccnn12]

To a solution of N-(imidazo[1,2-b]pyridazin-3-yl)-6-methoxy-2-((1r,4r)-4-(piperazin-1-yl)cyclohexyl)-2H-indazole-5-carboxamide (40 mg, 0.084 mmol) (obtained through the similar way described in WO2022122876A1) and (R)-1-(4-(2,6-dioxopiperidin-3-yl)-3,5-difluorophenyl)azetidine-3-carboxylic acid (27.34 mg, 0.084 mmol) in DCM (5 mL) was added DIPEA (32.51 mg, 0.252 mmol) and T₃P in EtOAc (50%, 106.91 mg, 0.168 mmol) at rt. The resulting mixture was stirred for 2 hrs at rt. The mixture was diluted with water and extracted with DCM. The combined organic layers were dried over anhydrous Na₂SO₄, filtered and evaporated in vacuum. The residue was purified by Prep-HPLC (C-18 column chromatography (0.1% FA in water:acetonitrile=90:10˜60:40 gradient elution) to give the desired product (40 mg, 30.4%). ¹H NMR (500 MHz, DMSO) δ 11.05 (s, 1H), 10.86 (s, 1H), 8.67-8.63 (m, 1H), 8.58 (s, 2H), 8.17-8.13 (m, 1H), 8.05 (s, 1H), 7.25 (s, 1H), 7.22 (dd, J=9.2, 4.4 Hz, 1H), 6.17 (d, J=11.1 Hz, 2H), 4.46 (t, J=11.6 Hz, 1H), 4.12 (s, 3H), 4.07-4.00 (m, 3H), 3.92 (t, J=6.3 Hz, 2H), 3.87-3.79 (m, 1H), 3.55-3.41 (m, 3H), 3.32-3.16 (m, 3H), 2.84-2.72 (m, 1H), 2.63-2.52 (m, 4H), 2.21 (d, J=9.4 Hz, 2H), 2.14-2.03 (m, 1H), 1.94 (t, J=11.0 Hz, 5H), 1.60-1.42 (m, 2H). [M+H]⁺=781.3.

### Example 157: (R)-3-cyano-N-(2-(3-(4-(1-(4-(2,6-dioxopiperidin-3-yl)-3,5-difluorophenyl)azetidine-3-carbonyl)piperazin-1-yl)bicyclo[1.1.1]pentan-1-yl)-6-(2-hydroxypropan-2-yl)-2H-indazol-5-yl)pyrrolo[1,2-b]pyridazine-7-carboxamide

### Step 1: tert-butyl (3-(4-benzylpiperazin-1-yl)bicyclo[1.1.1]pentan-1-yl)carbamate

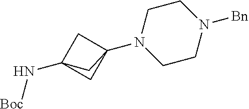 [SMILES: CC(C)(C)OC(=O)NC12CC(N3CCN(Cc4ccccc4)CC3)(C1)C2]

To a solution of tert-butyl (3-aminobicyclo[1.1.1]pentan-1-yl)carbamate (20 g, 101 mmol) in EtOH (300 mL) was added N-benzyl-2-chloro-N-(2-chloroethyl)ethan-1-amine hydrogen chloride (40.2 g, 150 mmol) and NaHCO₃ (29.4 g, 350 mmol). The mixture was stirred for 16 hrs at 80° C. under N₂. The mixture was stirred overnight at 80° C. under N₂. The mixture was cooled to rt, filtered and the filtrate was concentrated in vacuo. The residue was purified by silica gel column chromatography (PE:EA=5:1 to 1:1) to give product (22 g, 61%). [M+H]⁺=358.2.

### Step 2: 3-(4-benzylpiperazin-1-yl)bicyclo[1.1.1]pentan-1-amine

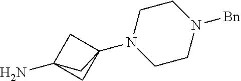 [SMILES: NC12CC(N3CCN(Cc4ccccc4)CC3)(C1)C2]

To a solution of tert-butyl (3-(4-benzylpiperazin-1-yl)bicyclo[1.1.1]pentan-1-yl)carbamate (22 g, 61.59 mmol) in DCM (150 mL) was added TFA (40 mL) at rt. The mixture was stirred for 3 hrs at rt under N₂. The mixture was concentrated in vacuo then adjusted to pH 9 with sat. Na₂CO₃ solution and extracted with DCM. The combined organic layers were dried over anhydrous Na₂SO₄, filtered and evaporated in vacuum to afford crude product (13 g, 82%). [M+H]⁺=258.2.

### Step 3: methyl 2-(3-(4-benzylpiperazin-1-yl)bicyclo[1.1.1]pentan-1-yl)-2H-indazole-6-carboxylate

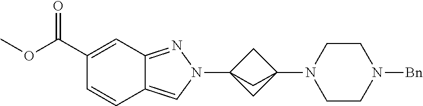 [SMILES: COC(=O)c1ccc2cn(C34CC(N5CCN(Cc6ccccc6)CC5)(C3)C4)nc2c1]

To a solution of methyl 4-formyl-3-nitrobenzoate (10.56 g, 50 mmol) in propan-2-ol (150 mL) was added 3-(4-benzylpiperazin-1-yl)bicyclo[1.1.1]pentan-1-amine (13 g, 50 mmol). The mixture was stirred for 3 hrs at 80° C. under N₂. To the above solution was added tributylphosphane (20.36 g, 100.8 mmol). The mixture was stirred overnight at 80° C. under N₂. The mixture was cooled to rt and concentrated in vacuo. The residue was purified by silica gel column chromatography (PE:EA=5:1 to 1:1) to give product (7.5 g, 35.7%). [M+H]⁺=417.2.

### Step 4: methyl 2-(3-(piperazin-1-yl)bicyclo[1.1.1]pentan-1-yl)-2H-indazole-6-carboxylate

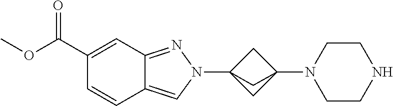 [SMILES: COC(=O)c1ccc2cn(C34CC(N5CCNCC5)(C3)C4)nc2c1]

To a solution of methyl 2-(3-(4-benzylpiperazin-1-yl)bicyclo[1.1.1]pentan-1-yl)-2H-indazole-6-carboxylate (7.5 g, 18 mmol) in DCM (50 mL) was added 1-chloroethyl carbonochloridate (3.86 g, 27 mmol). The mixture was stirred for 3 hrs at rt under N₂. The mixture was concentrated in vacuo. The residue was dissolved in MeOH (50 mL). The mixture was heated to 70° C. and stirred for 2 hrs. The mixture was cooled to rt and concentrated in vacuo. The residue was purified by silica gel column chromatography (PE:EA=5:1 to 1:1) to give product (4.7 g, 80%). [M+H]⁺=327.2.

### Step 5: methyl 5-nitro-2-(3-(piperazin-1-yl)bicyclo[1.1.1]pentan-1-yl)-2H-indazole-6-carboxylate

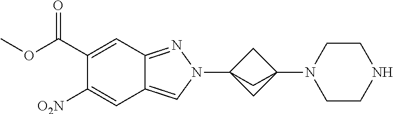 [SMILES: COC(=O)c1cc2nn(C34CC(N5CCNCC5)(C3)C4)cc2cc1[N+](=O)[O-]]

To a solution of methyl 2-(3-(piperazin-1-yl)bicyclo[1.1.1]pentan-1-yl)-2H-indazole-6-carboxylate (4.5 g, 13.80 mmol) in conc. H₂SO₄ (25 mL) was slowed added NaNO₃ (1.41 g, 16.56 mmol) at 0° C. The mixture was stirred for 2 hrs at 0° C. The mixture was quenched with ice-water, then adjusted to pH 9 with sat.Na₂CO₃ solution and extracted with DCM. The combined organic layers were dried over anhydrous Na₂SO₄, filtered and evaporated in vacuum to afford product (4.3 g, 84%), which was used without further purification. [M+H]⁺=372.2.

### Step 6: methyl 2-(3-(4-(tert-butoxycarbonyl)piperazin-1-yl)bicyclo[1.1.1]pentan-1-yl)-5-nitro-2H-indazole-6-carboxylate

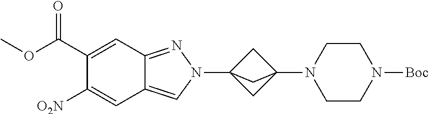 [SMILES: COC(=O)c1cc2nn(C34CC(N5CCN(C(=O)OC(C)(C)C)CC5)(C3)C4)cc2cc1[N+](=O)[O-]]

To a stirred solution of methyl 5-nitro-2-(3-(piperazin-1-yl)bicyclo[1.1.1]pentan-1-yl)-2H-indazole-6-carboxylate (4.3 g, 11.6 mmol) in DCM (60 mL) was added Et₃N (2.34 g, 23.2 mmol) and Boc₂O (3.04 g, 13.92 mmol). The reaction mixture was stirred overnight at rt. The mixture was concentrated in vacuo. The residue was purified by silica gel column chromatography (PE:EA=3:1 to 1:1) to give product (4.75 g, 87%). [M+H]⁺=472.2.

### Step 7: methyl 5-amino-2-(3-(4-(tert-butoxycarbonyl)piperazin-1-yl)bicyclo[1.1.1]pentan-1-yl)-2H-indazole-6-carboxylate

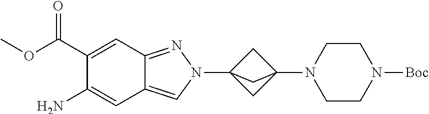 [SMILES: COC(=O)c1cc2nn(C34CC(N5CCN(C(=O)OC(C)(C)C)CC5)(C3)C4)cc2cc1N]

To a solution of methyl 2-(3-(4-(tert-butoxycarbonyl)piperazin-1-yl)bicyclo[1.1.1]pentan-1-yl)-5-nitro-2H-indazole-6-carboxylate (4.5 g, 9.5 mmol) in MeOH (50 mL) and water (10 mL) was added NH₄Cl (5.08 g, 95 mmol) and Fe powder (2.66 g, 47.5 mmol) at room temperature. The resulting mixture was stirred for 3 hrs at 70° C. The mixture was cooled to rt and concentrated in vacuo. The residue was purified by silica gel column chromatography (DCM:MeOH=15:1) to give product (3.03 g, 72%). [M+H]⁺=442.3.

### Step 8: methyl 2-(3-(4-(tert-butoxycarbonyl)piperazin-1-yl)bicyclo[1.1.1]pentan-1-yl)-5-(2,2,2-trifluoroacetamido)-2H-indazole-6-carboxylate

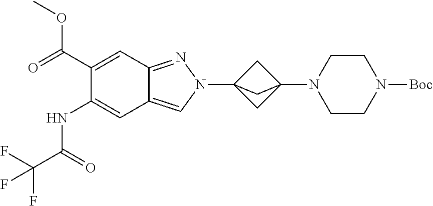 [SMILES: COC(=O)c1cc2nn(C34CC(N5CCN(C(=O)OC(C)(C)C)CC5)(C3)C4)cc2cc1NC(=O)C(F)(F)F]

To a solution of methyl 5-amino-2-(3-(4-(tert-butoxycarbonyl)piperazin-1-yl)bicyclo[1.1.1]pentan-1-yl)-2H-indazole-6-carboxylate (3 g, 6.8 mmol) in DCM (30 mL) was added dropwise TFAA (1.71 g, 8.16 mmol) at 0° C. The resulting mixture was stirred for 2 hrs at 0° C. The mixture was quenched with sat.NH₄Cl solution and extracted with DCM. The combined organic layers were dried over anhydrous Na₂SO₄, filtered and evaporated in vacuum. The residue was purified by silica gel column chromatography (DCM:MeOH=10:1) to give product (3.1 g, 85%). [M+H]⁺=538.2.

### Step 9: tert-butyl 4-(3-(6-(2-hydroxypropan-2-yl)-5-(2,2,2-trifluoroacetamido)-2H-indazol-2-yl)bicyclo[1.1.1]pentan-1-yl)piperazine-1-carboxylate

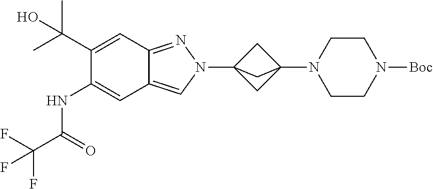 [SMILES: CC(C)(C)OC(=O)N1CCN(C23CC(n4cc5cc(NC(=O)C(F)(F)F)c(C(C)(C)O)cc5n4)(C2)C3)CC1]

To a solution of methyl 2-(3-(4-(tert-butoxycarbonyl)piperazin-1-yl)bicyclo[1.1.1]pentan-1-yl)-5-(2,2,2-trifluoroacetamido)-2H-indazole-6-carboxylate (3.1 g, 5.8 mmol) in THF (30 mL) was added MeMgBr (3M in THF) (19.3 mL, 58 mmol) at 0° C. The resulting mixture was stirred for 5 hrs at rt. The mixture was quenched with sat.NH₄Cl and extracted with DCM. The combined organic layers were dried over anhydrous Na₂SO₄, filtered and evaporated in vacuum. The residue was purified by silica gel column chromatography (DCM:MeOH=20:1) to give product (2.54 g, 82%). [M+H]⁺=538.2.

### Step 10: tert-butyl 4-(3-(5-amino-6-(2-hydroxypropan-2-yl)-2H-indazol-2-yl)bicyclo[1.1.1]pentan-1-yl)piperazine-1-carboxylate

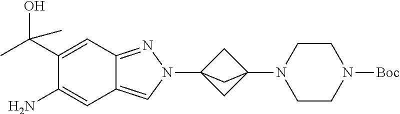 [SMILES: CC(C)(C)OC(=O)N1CCN(C23CC(n4cc5cc(N)c(C(C)(C)O)cc5n4)(C2)C3)CC1]

To a solution of tert-butyl 4-(3-(6-(2-hydroxypropan-2-yl)-5-(2,2,2-trifluoroacetamido)-2H-indazol-2-yl)bicyclo[1.1.1]pentan-1-yl)piperazine-1-carboxylate (2.5 g, 5.66 mmol) in MeOH (20 mL) was added NaOH (2.26 g, 56.6 mmol) in water (15 mL) at rt. The resulting mixture was stirred for 2 hrs at 70° C. The mixture was diluted with water and extracted with DCM. The combined organic layers were dried over anhydrous Na₂SO₄, filtered and evaporated in vacuum. The residue was purified by silica gel column chromatography (DCM:MeOH=20:1) to give product (1.52 g, 74%). [M+H]⁺=442.3.

### Step 11: tert-butyl 4-(3-(5-(3-cyanopyrrolo[1,2-b]pyridazine-7-carboxamido)-6-(2-hydroxypropan-2-yl)-2H-indazol-2-yl)bicyclo[1.1.1]pentan-1-yl)piperazine-1-carboxylate

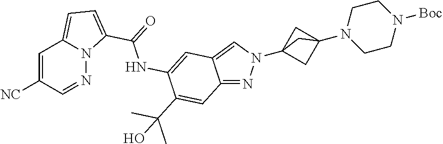 [SMILES: CC(C)(C)OC(=O)N1CCN(C23CC(n4cc5cc(NC(=O)c6ccc7cc(C#N)cnn67)c(C(C)(C)O)cc5n4)(C2)C3)CC1]

To a solution of tert-butyl 4-(3-(5-amino-6-(2-hydroxypropan-2-yl)-2H-indazol-2-yl)bicyclo[1.1.1]pentan-1-yl)piperazine-1-carboxylate (1.5 g, 3.40 mmol) in DMF (20 mL) was added 3-cyanopyrrolo[1,2-b]pyridazine-7-carboxylic acid (635 mg, 3.40 mmol), TEA (687 mg, 6.80 mmol), HATU (1.55 mg, 4.08 mmol) at rt. The resulting mixture was stirred for 14 hrs at rt. The mixture was diluted with water and extracted with DCM. The combined organic layers were dried over anhydrous Na₂SO₄, filtered and evaporated in vacuum. The residue was purified by silica gel column chromatography (DCM:MeOH=20:1) to give product (1.55 g, 75%). [M+H]⁺=611.4.

### Step 12: 3-cyano-N-(6-(2-hydroxypropan-2-yl)-2-(3-(piperazin-1-yl)bicyclo[1.1.1]pentan-1-yl)-2H-indazol-5-yl)pyrrolo[1,2-b]pyridazine-7-carboxamide

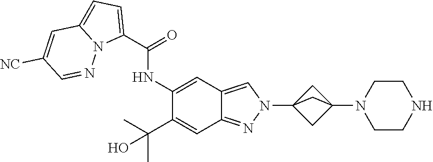 [SMILES: CC(C)(O)c1cc2nn(C34CC(N5CCNCC5)(C3)C4)cc2cc1NC(=O)c1ccc2cc(C#N)cnn12]

To a solution of tert-butyl 4-(3-(5-(3-cyanopyrrolo[1,2-b]pyridazine-7-carboxamido)-6-(2-hydroxypropan-2-yl)-2H-indazol-2-yl)bicyclo[1.1.1]pentan-1-yl)piperazine-1-carboxylate (1.5 g, 2.45 mmol) in DCM (10 mL) was added MeOH (10 mL) and con. HCl (10 mL) at 0° C. The resulting mixture was stirred for 2 h at rt. The mixture was quenched with sat.Na2CO₃ solution and extracted with DCM. The combined organic layers were dried over anhydrous Na₂SO₄, filtered and evaporated in vacuum to give product (1.1 g, 88%). [M+H]⁺=511.3.

### Step 13: (R)-3-cyano-N-(2-(3-(4-(1-(4-(2,6-dioxopiperidin-3-yl)-3,5-difluorophenyl)azetidine-3-carbonyl)piperazin-1-yl)bicyclo[1.1.1]pentan-1-yl)-6-(2-hydroxypropan-2-yl)-2H-indazol-5-yl)pyrrolo[1,2-b]pyridazine-7-carboxamide

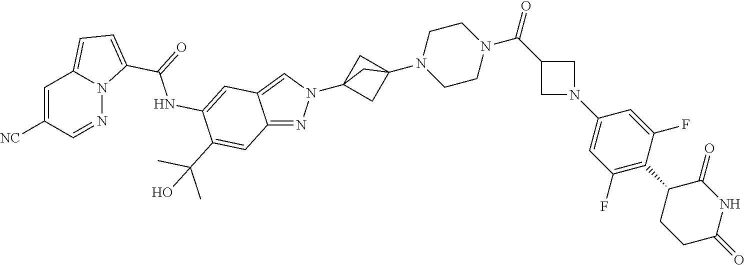 [SMILES: CC(C)(O)c1cc2nn(C34CC(N5CCN(C(=O)C6CN(c7cc(F)c([C@H]8CCC(=O)NC8=O)c(F)c7)C6)CC5)(C3)C4)cc2cc1NC(=O)c1ccc2cc(C#N)cnn12]

To a solution of 3-cyano-N-(6-(2-hydroxypropan-2-yl)-2-(3-(piperazin-1-yl)bicyclo[1.1.1]pentan-1-yl)-2H-indazol-5-yl)pyrrolo[1,2-b]pyridazine-7-carboxamide (40 mg, 0.078 mmol) and (R)-1-(4-(2,6-dioxopiperidin-3-yl)-3,5-difluorophenyl)azetidine-3-carboxylic acid (25.4 mg, 0.078 mmol) in DCM (5 mL) was added DIPEA (28.6 mg, 0.22 mmol) and T₃P in EtOAc (50%, 100 mg, 0.156) mmol) at rt. The resulting mixture was stirred for 2 hrs at rt. The mixture was diluted with water and extracted with DCM. The combined organic layers were dried over anhydrous Na₂SO₄, filtered and evaporated in vacuum. The residue was purified by Prep-HPLC (C-18 column chromatography (0.1% FA in water:acetonitrile=90:10˜60:40 gradient elution) to give the desired product (30 mg, 46.8%). ¹H NMR (500 MHz, DMSO) δ 12.06 (s, 1H), 10.86 (s, 1H), 8.93 (d, J=2.1 Hz, 1H), 8.74 (d, J=2.1 Hz, 1H), 8.59 (s, 1H), 8.37 (s, 1H), 7.72 (d, J=4.8 Hz, 1H), 7.60 (s, 1H), 7.09 (d, J=4.8 Hz, 1H), 6.18 (d, J=11.0 Hz, 2H), 5.78 (s, 1H), 4.10-4.00 (m, 3H), 3.93 (t, J=6.1 Hz, 2H), 3.85 (dt, J=15.0, 7.5 Hz, 1H), 3.59-3.51 (m, 2H), 3.44-3.35 (m, 4H), 2.83-2.72 (m, 1H), 2.48-2.43 (m, 3H), 2.35 (s, 6H), 2.13-2.01 (m, 1H), 1.99-1.89 (m, 1H), 1.63 (s, 6H). [M+H]⁺=817.3.

### Example 332: (R)-3-cyano-N-(2-(3-(4-((1-(4-(2,6-dioxopiperidin-3-yl)-3,5-difluorophenyl)azetidin-3-yl)methyl)piperazin-1-yl)bicyclo[1.1.1]pentan-1-yl)-6-(2-hydroxypropan-2-yl)-2H-indazol-5-yl)pyrrolo[1,2-b]pyridazine-7-carboxamide

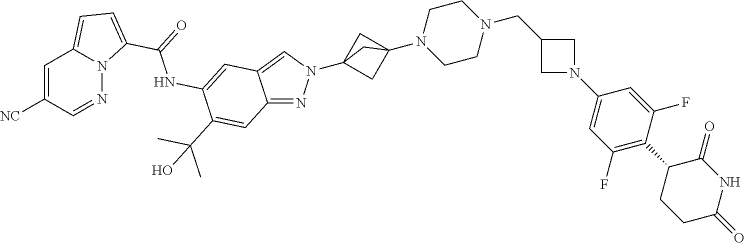 [SMILES: CC(C)(O)c1cc2nn(C34CC(N5CCN(CC6CN(c7cc(F)c([C@H]8CCC(=O)NC8=O)c(F)c7)C6)CC5)(C3)C4)cc2cc1NC(=O)c1ccc2cc(C#N)cnn12]

To a solution of 3-cyano-N-(6-(2-hydroxypropan-2-yl)-2-(3-(piperazin-1-yl)bicyclo[11.1.1]pentan-1-yl)-2H-indazol-5-yl)pyrrolo[1,2-b]pyridazine-7-carboxamide (40 mg, 0.078 mmol) and (R)-1-(4-(2,6-dioxopiperidin-3-yl)-3,5-difluorophenyl)azetidine-3-carbaldehyde (24.2 mg, 0.078 mmol) in DCE (5 mL) was was added NaBH(OAc)₃ (33.2 mg, 0.157 mmol) at rt. The resulting mixture was stirred for 12 hrs at rt. The mixture was diluted with water and extracted with DCM. The combined organic layers were dried over anhydrous Na₂SO₄, filtered and evaporated in vacuum. The residue was purified by Prep-HPLC (C-18 column chromatography (0.100 FA in water:acetonitrile=90:10˜60:40 gradient elution) to give the desired product (29.6 mg, 47.0%). ¹H NMR (500 MHz, DMSO) δ 12.05 (s, 1H), 10.85 (s, 1H), 8.93 (d, J=2.1 Hz, 1H), 8.74 (d, J=2.1 Hz, 1H), 8.59 (s, 1H), 8.35 (s, 1H), 7.72 (d, J=4.8 Hz, 1H), 7.60 (s, 1H), 7.09 (d, J=4.8 Hz, 1H), 6.16-6.07 (d, J=11.2 Hz, 2H), 5.78 (s, 1H), 4.02 (dd, J=12.5, 4.8 Hz, 1H), 3.93 (t, J=7.5 Hz, 2H), 3.48 (t, J=5.8 Hz, 2H), 2.97-2.87 (m, 1H), 2.82-2.72 (m, 1H), 2.58 (d, J=7.4 Hz, 2H), 2.48-2.37 (m, 6H), 2.33 (s, 6H), 2.12-2.02 (m, 1H), 1.99-1.91 (m, 1H), 1.63 (s, 6H). [M+H]⁺=803.4.

### Example 159: (R)-3-cyano-N-(2-(3-(4-(4-(2,6-dioxopiperidin-3-yl)-3,5-difluorophenethyl)piperazin-1-yl)bicyclo[1.1.1]pentan-1-yl)-6-(2-hydroxypropan-2-yl)-2H-indazol-5-yl)pyrrolo[1,2-b]pyridazine-7-carboxamide

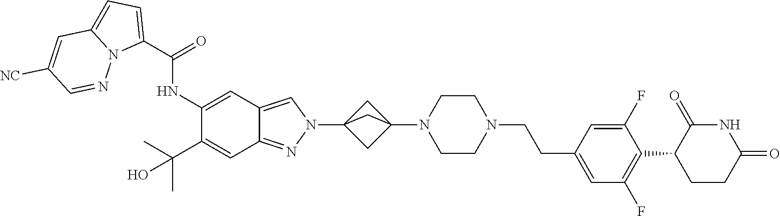 [SMILES: CC(C)(O)c1cc2nn(C34CC(N5CCN(CCc6cc(F)c([C@H]7CCC(=O)NC7=O)c(F)c6)CC5)(C3)C4)cc2cc1NC(=O)c1ccc2cc(C#N)cnn12]

The title compound (24 mg, 40.2%) was prepared in a manner similar to that described in Example 332. ¹H NMR (500 MHz, DMSO) δ 12.05 (s, 1H), 10.94 (s, 1H), 8.93 (d, J=2.1 Hz, 1H), 8.73 (d, J=2.1 Hz, 1H), 8.59 (s, 1H), 8.35 (s, 1H), 7.72 (d, J=4.8 Hz, 1H), 7.60 (s, 1H), 7.09 (d, J=4.8 Hz, 1H), 7.03 (d, J=10.0 Hz, 2H), 5.78 (s, 1H), 4.20 (dd, J=12.7, 5.0 Hz, 1H), 3.42-3.36 (m, 2H), 2.86-2.74 (m, 3H), 2.63-2.51 (m, 6H), 2.47-2.38 (m, 3H), 2.33 (s, 6H), 2.18-2.06 (m, 1H), 2.03-1.97 (m, 1H), 1.63 (s, 6H). [M+H]⁺=762.3.

### Example 254: 3-cyano-N-(2-((1r,4r)-4-(4-(1-(6-(2,6-dioxopiperidin-3-yl)pyridin-3-yl)piperidin-4-yl)piperazin-1-yl)cyclohexyl)-6-(2-hydroxypropan-2-yl)-2H-indazol-5-yl)pyrrolo[1,2-b]pyridazine-7-carboxamide

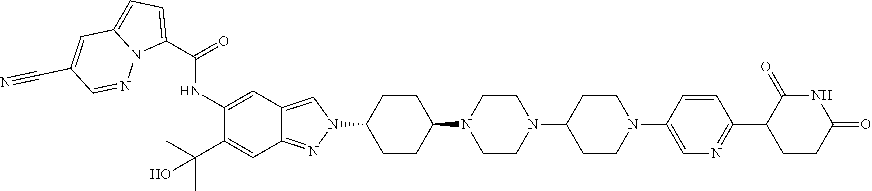 [SMILES: CC(C)(O)c1cc2nn([C@H]3CC[C@H](N4CCN(C5CCN(c6ccc(C7CCC(=O)NC7=O)nc6)CC5)CC4)CC3)cc2cc1NC(=O)c1ccc2cc(C#N)cnn12]

The title compound (18 mg, 29.7%) was prepared in a manner similar to that described in Example 290. ¹H NMR (500 MHz, DMSO) δ 12.03 (s, 1H), 10.78 (s, 1H), 8.94 (d, J=1.9 Hz, 1H), 8.75 (s, 1H), 8.55 (s, 1H), 8.34 (s, 1H), 8.21 (s, 1H), 7.72 (d, J=4.7 Hz, 1H), 7.57 (s, 1H), 7.33 (dd, J=8.9, 1.9 Hz, 1H), 7.17 (d, J=8.9 Hz, 1H), 7.09 (d, J=4.7 Hz, 1H), 5.74 (s, 1H), 4.44 (t, J=14.7 Hz, 1H), 3.90-3.86 (m, 1H), 3.79 (d, J=10.7 Hz, 2H), 2.83-2.68 (m, 9H), 2.65-2.54 (m, 5H), 2.24-2.15 (m, 3H), 2.13-2.02 (m, 3H), 1.98-1.88 (m, 4H), 1.63 (s, 6H), 1.59-1.49 (in, 4H). [M+H]⁺=798.6.

### Example 255: 3-cyano-N-(2-((1r,4r)-4-(4-(1-(6-(2,6-dioxopiperidin-3-yl)pyridin-3-yl)azetidine-3-carbonyl)piperazin-1-yl)cyclohexyl)-6-(2-hydroxypropan-2-yl)-2H-indazol-5-yl)pyrrolo[1,2-b]pyridazine-7-carboxamide

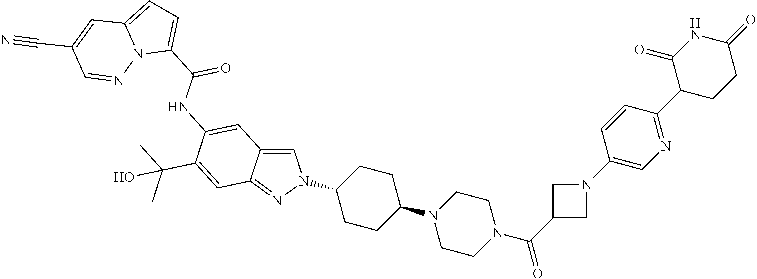 [SMILES: CC(C)(O)c1cc2nn([C@H]3CC[C@H](N4CCN(C(=O)C5CN(c6ccc(C7CCC(=O)NC7=O)nc6)C5)CC4)CC3)cc2cc1NC(=O)c1ccc2cc(C#N)cnn12]

The title compound (18 mg, 29%) was prepared in a manner similar to that described in Example 178. ¹H NMR (500 MHz, DMSO) δ 12.02 (s, 1H), 10.76 (s, 1H), 8.93 (d, J=2.0 Hz, 1H), 8.74 (d, J=2.0 Hz, 1H), 8.55 (s, 1H), 8.34 (s, 1H), 7.76 (d, J=2.5 Hz, 1H), 7.72 (d, J=4.7 Hz, 1H), 7.57 (s, 1H), 7.14 (d, J=8.4 Hz, 1H), 7.09 (d, J=4.7 Hz, 1H), 6.86 (dd, J=8.4, 2.5 Hz, 1H), 5.72 (s, 1H), 4.43 (t, J=9.7 Hz, 1H), 4.08 (t, J=7.6 Hz, 2H), 3.94 (t, J=6.5 Hz, 2H), 3.90-3.83 (m, 2H), 3.53-3.45 (m, 2H), 2.61-2.52 (m, 6H), 2.48-2.35 (m, 3H), 2.23-2.14 (m, 3H), 2.12-2.06 (m, 1H), 2.00-1.90 (m, 4H), 1.63 (s, 6H), 1.55-1.45 (m, 2H). [M+H]⁺=798.6.

### Example 257: 3-cyano-N-(2-((1r,4r)-4-(4-(1-(4-(2,4-dioxotetrahydropyrimidin-1(2H)-yl)-3-ethylphenyl)azetidine-3-carbonyl)piperazin-1-yl)cyclohexyl)-6-(2-hydroxypropan-2-yl)-2H-indazol-5-yl)pyrrolo[1,2-b]pyridazine-7-carboxamide

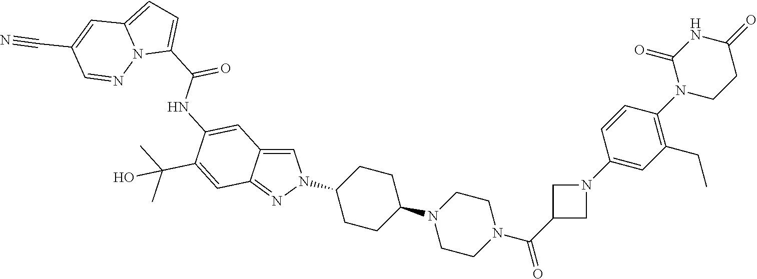 [SMILES: CCc1cc(N2CC(C(=O)N3CCN([C@H]4CC[C@H](n5cc6cc(NC(=O)c7ccc8cc(C#N)cnn78)c(C(C)(C)O)cc6n5)CC4)CC3)C2)ccc1N1CCC(=O)NC1=O]

The title compound (23 mg, 29.26%) was prepared in a manner similar to that described in Example 178. ¹H NMR (500 MHz, DMSO) δ 12.02 (s, 1H), 10.23 (s, 1H), 8.93 (d, J=2.1 Hz, 1H), 8.74 (d, J=2.1 Hz, 1H), 8.55 (s, 1H), 8.34 (s, 1H), 7.72 (d, J=4.8 Hz, 1H), 7.57 (s, 1H), 7.09 (d, J=4.8 Hz, 1H), 7.03 (d, J=8.3 Hz, 1H), 6.35-6.29 (m, 2H), 5.72 (s, 1H), 4.43 (t, J=11.3 Hz, 1H), 4.03 (t, J=7.5 Hz, 2H), 3.88 (t, J=6.5 Hz, 2H), 3.85-3.78 (m, 1H), 371-3.65 (m, 1H), 3.51-3.46 (m, 2H), 3.44-3.38 (m, 1H), 2.77-2.61 (m, 2H), 2.58-2.51 (m, 6H), 2.49-2.41 (m, 3H), 2.22-2.15 (m, 2H), 2.01-1.89 (m, 4H), 1.63 (s, 6H), 1.50 (q, J=12.0 Hz, 2H), 1.12 (t, J=7.6 Hz, 3H). [M+H]⁺=826.6.

### Example 258: 3-cyano-N-(2-((1r,4r)-4-(4-(1-(4-(2,4-dioxotetrahydropyrimidin-1(2H)-yl)-2-fluoro-5-methoxyphenyl)azetidine-3-carbonyl)piperazin-1-yl)cyclohexyl)-6-(2-hydroxypropan-2-yl)-2H-indazol-5-yl)pyrrolo[1,2-b]pyridazine-7-carboxamide

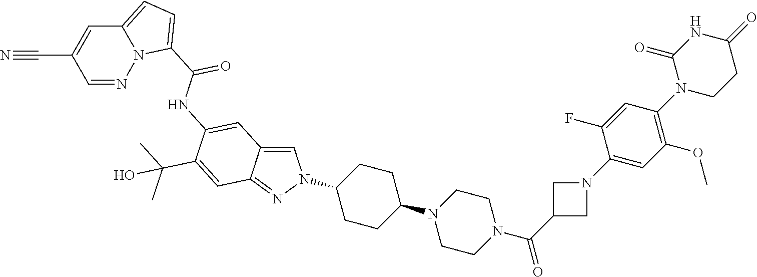 [SMILES: COc1cc(N2CC(C(=O)N3CCN([C@H]4CC[C@H](n5cc6cc(NC(=O)c7ccc8cc(C#N)cnn78)c(C(C)(C)O)cc6n5)CC4)CC3)C2)c(F)cc1N1CCC(=O)NC1=O]

The title compound (12 mg, 14.93%) was prepared in a manner similar to that described in Example 178. ¹H NMR (500 MHz, DMSO) δ 12.02 (s, 1H), 10.25 (s, 1H), 8.93 (d, J=2.1 Hz, 1H), 8.74 (d, J=2.1 Hz, 1H), 8.55 (s, 1H), 8.33 (s, 1H), 7.72 (d, J=4.8 Hz, 1H), 7.57 (s, 1H), 7.09 (d, J=4.8 Hz, 1H), 7.01 (d, J=12.6 Hz, 1H), 6.22 (d, J=8.5 Hz, 1H), 5.72 (s, 1H), 4.43 (t, J=11.5 Hz, 1H), 4.14 (t, J=7.1 Hz, 2H), 4.02 (t, J=6.4 Hz, 2H), 3.88-3.80 (m, 1H), 3.77 (s, 3H), 3.55-3.46 (m, 4H), 2.64 (t, J=6.6 Hz, 2H), 2.58-2.51 (m, 7H), 2.21-2.14 (m, 2H), 2.01-1.88 (m, 4H), 1.63 (s, 6H), 1.56-1.44 (m, 2H). [M+H]+=846.6.

### Example 288: 3-cyano-N-(2-((R)-1′-(1-(4-((R)-2,6-dioxopiperidin-3-yl)-3,5-difluorophenyl)azetidine-3-carbonyl)-3′,3′-difluoro-[1,4′-bipiperidin]-4-yl)-6-(2-hydroxypropan-2-yl)-2H-indazol-5-yl)pyrrolo[1,2-b]pyridazine-7-carboxamide

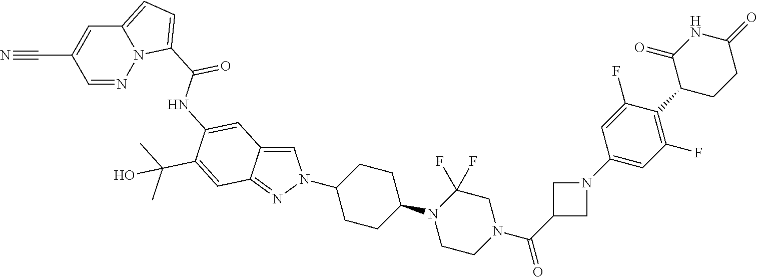

The title compound (30 mg, 38.20%) was prepared in a manner similar to that described in Example 178. ¹H NMR (500 MHz, DMSO) δ 12.03 (s, 1H), 10.86 (s, 1H), 8.93 (d, J=2.1 Hz, 1H), 8.74 (d, J=2.1 Hz, 1H), 8.56 (s, 1H), 8.38 (s, 1H), 7.72 (d, J=4.8 Hz, 1H), 7.58 (s, 1H), 7.09 (d, J=4.8 Hz, 1H), 6.18 (t, J=11.1 Hz, 2H), 5.73 (s, 1H), 4.63-4.42 (m, 2H), 4.11-4.01 (m, 3H), 3.94-3.87 (m, 3H), 3.85-3.71 (m, 1H), 3.50-3.37 (m, 1H), 3.26-2.96 (m, 5H), 2.88-2.61 (m, 4H), 2.15-2.01 (m, 5H), 1.97-1.91 (m, 1H), 1.88-1.82 (m, 1H), 1.63 (s, 6H). [M+H]⁺=869.6.

### Example 290: 3-cyano-N-(2-((1r,4r)-4-(4-((1-(4-((R)-2,6-dioxopiperidin-3-yl)-3,5-difluorophenyl)azetidin-3-yl)methyl)piperazin-1-yl)cyclohexyl)-6-(2-hydroxypropan-2-yl)-2H-indazol-5-yl)pyrrolo[1,2-b]pyridazine-7-carboxamide

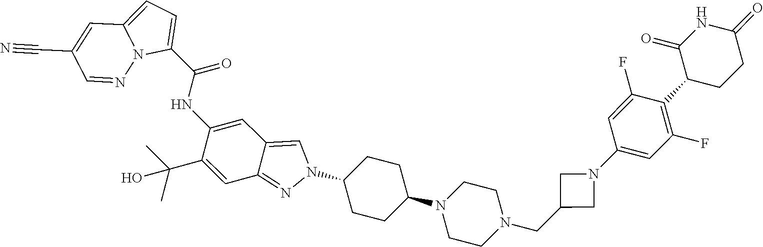 [SMILES: CC(C)(O)c1cc2nn([C@H]3CC[C@H](N4CCN(CC5CN(c6cc(F)c([C@H]7CCC(=O)NC7=O)c(F)c6)C5)CC4)CC3)cc2cc1NC(=O)c1ccc2cc(C#N)cnn12]

To a solution of 3-cyano-N-(6-(2-hydroxypropan-2-yl)-2-((1r,4r)-4-(piperazin-1-yl)cyclohexyl)-2H-indazol-5-yl)pyrrolo[1,2-b]pyridazine-7-carboxamide (50 mg, 0.095 mmol) and (R)-1-(4-(2,6-dioxopiperidin-3-yl)-3,5-difluorophenyl)azetidine-3-carbaldehyde (43.9 mg, 0.142 mmol) in DCE (2 mL) was added sodium triacetoxyborohydride (40.24 mg, 0.190 mmol) at rt. The mixture was stirred at rt for 3 h. Then the mixture was diluted with DCM (15 mL), washed with saturated aqueous sodium bicarbonate (10 mL) and brine (10 mL). The organic layer was dried over anhydrous sodium sulfate and filtered. The filtrate was concentrated in vacuo. The residue was purified by silica column (DCM:MeOH=10:1) and prep-HPLC (C-18 column chromatography (0.1% FA in water:acetonitrile=90:10˜60:40 gradient elution) to give 3-cyano-N-(2-((1r,4r)-4-(4-((1-(4-((R)-2,6-dioxopiperidin-3-yl)-3,5-difluorophenyl)azetidin-3-yl)methyl)piperazin-1-yl)cyclohexyl)-6-(2-hydroxypropan-2-yl)-2H-indazol-5-yl)pyrrolo[1,2-b]pyridazine-7-carboxamide (20 mg, 25.71%). ¹H NMR (500 MHz, DMSO) δ 12.02 (s, 1H), 10.85 (s, 1H), 8.93 (d, J=2.1 Hz, 1H), 8.74 (d, J=2.1 Hz, 1H), 8.55 (s, 1H), 8.33 (s, 1H), 7.72 (d, J=4.8 Hz, 1H), 7.57 (s, 1H), 7.09 (d, J=4.8 Hz, 1H), 6.10 (d, J=11.1 Hz, 2H), 5.72 (s, 1H), 4.42 (t, J=11.6 Hz, 1H), 4.02 (dd, J=12.5, 5.0 Hz, 1H), 3.93 (t, J=7.5 Hz, 2H), 3.47 (t, J=5.8 Hz, 2H), 2.97-2.86 (m, 1H), 2.82-2.72 (m, 1H), 2.63-2.52 (m, 6H), 2.49-2.29 (m, 6H), 2.20-2.15 (m, 2H), 2.16-2.02 (m, 1H), 2.00-1.88 (m, 5H), 1.63 (s, 6H), 1.52-1.41 (m, 2H). [M+H]⁺=819.6.

### Example 293: 3-cyano-N-(2-((1r,4r)-4-(4-(1-(4-(2,4-dioxotetrahydropyrimidin-1(2H)-yl)-3,5-difluorophenyl)piperidin-4-yl)piperazin-1-yl)cyclohexyl)-6-(2-hydroxypropan-2-yl)-2H-indazol-5-yl)pyrrolo[1,2-b]pyridazine-7-carboxamide

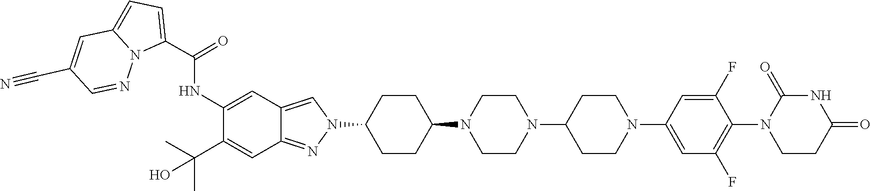 [SMILES: CC(C)(O)c1cc2nn([C@H]3CC[C@H](N4CCN(C5CCN(c6cc(F)c(N7CCC(=O)NC7=O)c(F)c6)CC5)CC4)CC3)cc2cc1NC(=O)c1ccc2cc(C#N)cnn12]

The title compound (16 mg, 20.21%) was prepared in a manner similar to that described in Example 290. ¹H NMR (500 MHz, DMSO) δ 12.02 (s, 1H), 10.50 (s, 1H), 8.93 (d, J=1.8 Hz, 1H), 8.74 (s, 1H), 8.55 (s, 1H), 8.33 (s, 1H), 7.72 (d, J=4.7 Hz, 1H), 7.57 (s, 1H), 7.09 (d, J=4.8 Hz, 1H), 6.72 (d, J=12.0 Hz, 2H), 5.72 (s, 1H), 4.42 (t, J=11.5 Hz, 1H), 3.81 (d, J=12.1 Hz, 2H), 3.60 (t, J=6.6 Hz, 2H), 2.77 (t, J=11.7 Hz, 2H), 2.68 (t, J=6.6 Hz, 2H), 2.60-2.51 (m, 8H), 2.40-2.32 (m, 2H), 2.20-2.13 (m, 2H), 2.00-1.88 (m, 4H), 1.85-1.78 (m, 2H), 1.63 (s, 6H), 1.46 (dd, J=26.6, 12.6 Hz, 4H). [M+H]⁺=834.6.

### Example 333: (R)-3-cyano-N-(2-(4-(4-(1-(4-(2,6-dioxopiperidin-3-yl)-3,5-difluorophenyl)piperidin-4-yl)piperazin-1-yl)bicyclo[2.2.2]octan-1-yl)-6-(2-hydroxypropan-2-yl)-2H-indazol-5-yl)pyrrolo[1,2-b]pyridazine-7-carboxamide

### Step 1: methyl 2-(4-((tert-butoxycarbonyl)amino)bicyclo[2.2.2]octan-1-yl)-2H-indazole-6-carboxylate

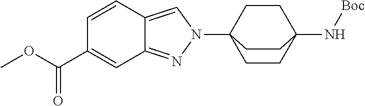 [SMILES: COC(=O)c1ccc2cn(C34CCC(NC(=O)OC(C)(C)C)(CC3)CC4)nc2c1]

To a solution of methyl 4-formyl-3-nitrobenzoate (7.87 g, 37.50 mmol) in propan-2-ol (100 mL) was added tert-butyl (4-aminobicyclo[2.2.2]octan-1-yl)carbamate (9 g, 37.50 mmol). The mixture was stirred for 3 hrs at 80° C. under N2. To the above solution was added tributylphosphane (22.72 g, 112.50 mmol). The mixture was stirred overnight at 80° C. under N₂. The mixture was cooled to rt and concentrated in vacuo. The residue was purified by silica gel column chromatography (PE:EA=3:1 to 1:1) to give product 12.0 g, 80%). [M+H]+=400.2

### Step 2: methyl 2-(4-aminobicyclo[2.2.2]octan-1-yl)-2H-indazole-6-carboxylate

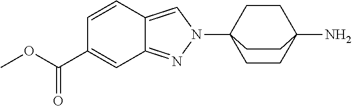 [SMILES: COC(=O)c1ccc2cn(C34CCC(N)(CC3)CC4)nc2c1]

To a solution of methyl 2-(4-((tert-butoxycarbonyl)amino)bicyclo[2.2.2]octan-1-yl)-2H-indazole-6-carboxylate (12.0 g, 30.0 mmol) in DCM (100 mL) was slowed added TFA (25 mL) at 0° C. The mixture was stirred for 2 hrs at RT. The mixture was concentrated. The residue was dissolved in DCM (100 mL), then adjusted to pH 9 with sat.Na₂CO₃ solution and extracted with DCM. The combined organic layers were dried over anhydrous Na₂SO₄, filtered and evaporated in vacuum to afford product (8.0 g, 89.9%), which was used without further purification. [M+H]⁺=300.2

### Step 3: methyl 2-(4-(4-benzylpiperazin-1-yl)bicyclo[2.2.2]octan-1-yl)-2H-indazole-6-carboxylate

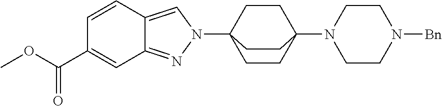 [SMILES: COC(=O)c1ccc2cn(C34CCC(N5CCN(Cc6ccccc6)CC5)(CC3)CC4)nc2c1]

To a stirred solution of methyl methyl 2-(4-aminobicyclo[2.2.2]octan-1-yl)-2H-indazole-6-carboxylate (8.0 g, 26.75 mmol) in ACN (200 mL) was added K2CO3 (14.76 g, 107 mmol) and N-benzyl-2-chloro-N-(2-chloroethyl)ethan-1-amine hydrogen chloride salt (10.78 g, 40.12 mmol), KI (1.76 g, 10.7 mmol). The reaction mixture was stirred overnight at 80° C. The mixture was concentrated in vacuo. The residue was purified by silica gel column chromatography (PE:EA=3:1 to 1:1) to give product (4.0 g, 32.6%). [M+H]⁺=459.3

### Step 4: methyl 2-(4-(piperazin-1-yl)bicyclo[2.2.2]octan-1-yl)-2H-indazole-6-carboxylate

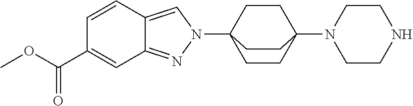 [SMILES: COC(=O)c1ccc2cn(C34CCC(N5CCNCC5)(CC3)CC4)nc2c1]

To a solution of methyl 2-(4-(4-benzylpiperazin-1-yl)bicyclo[2.2.2]octan-1-yl)-2H-indazole-6-carboxylate (4.0 g, 8.73 mmol) in DCM (40 mL) was added DIEA (1.35 g, 10.48 mmol) and 1-chloroethyl carbonochloridate (1.50 g, 10.48 mmol) at 0° C. The mixture was stirred for 12 hrs at RT. The mixture was concentrated. The residue was dissolved in MeOH (50 mL), then was stirred for 2 hrs at 80° C. The reaction mixture was directly concentrated. The residue was redissolved in water. The solution was adjusted to pH 9 with sat.Na₂CO₃ solution and extracted with DCM. The combined organic layers were dried over anhydrous Na₂SO₄, filtered and evaporated in vacuum to afford product (3.0 g, 93.7%), which was used without further purification. [M+H]⁺=369.2.

### Step 5: methyl 5-nitro-2-(4-(piperazin-1-yl)bicyclo[2.2.2]octan-1-yl)-2H-indazole-6-carboxylate

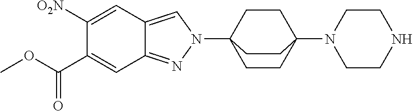 [SMILES: COC(=O)c1cc2nn(C34CCC(N5CCNCC5)(CC3)CC4)cc2cc1[N+](=O)[O-]]

To a solution of methyl 2-(4-(piperazin-1-yl)bicyclo[2.2.2]octan-1-yl)-2H-indazole-6-carboxylate (3.0 g, 8.15 mmol) in H₂SO₄ (20 mL) was slowly added NaNO3 (0.83 g, 9.78 mmol) at 0° C. The mixture was stirred for 2 hrs at RT. The mixture was poured into ice water. The solution was adjusted to pH 9 with Na₂CO₃ and extracted with DCM. The combined organic layers were dried over anhydrous Na₂SO₄, filtered and evaporated in vacuum to afford crude product (3.0 g, 89.0%), which was used without further purification. [M+H]⁺=414.2.

### Step 6: methyl 2-(4-(4-(tert-butoxycarbonyl)piperazin-1-yl)bicyclo[2.2.2]octan-1-yl)-5-nitro-2H-indazole-6-carboxylate

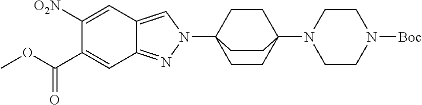 [SMILES: COC(=O)c1cc2nn(C34CCC(N5CCN(C(=O)OC(C)(C)C)CC5)(CC3)CC4)cc2cc1[N+](=O)[O-]]

To a solution of methyl 5-nitro-2-(4-(piperazin-1-yl)bicyclo[2.2.2]octan-1-yl)-2H-indazole-6-carboxylate (3.0 g, 7.26 mmol) in DCM (20 mL) was slowly added (Boc)₂O (1.9 g, 8.72 mmol) and TEA (1.47 g, 14.52 mmol) at RT. The mixture was stirred for 2 hrs at RT. The mixture was washed with water and extracted with DCM. The combined organic layers were dried over anhydrous Na₂SO₄, filtered and evaporated in vacuum. The residue was purified by silica gel column chromatography (PE:EA=3:1 to 1:1) to give product (2.0 g, 53.7%). [M+H]⁺=514.3

### Step 7: methyl 5-amino-2-(4-(4-(tert-butoxycarbonyl)piperazin-1-yl)bicyclo[2.2.2]octan-1-yl)-2H-indazole-6-carboxylate

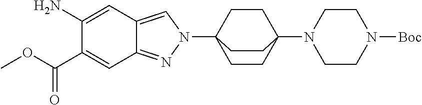 [SMILES: COC(=O)c1cc2nn(C34CCC(N5CCN(C(=O)OC(C)(C)C)CC5)(CC3)CC4)cc2cc1N]

To a solution of methyl 2-(4-(4-(tert-butoxycarbonyl)piperazin-1-yl)bicyclo[2.2.2]octan-1-yl)-5-nitro-2H-indazole-6-carboxylate (4.5 g, 8.77 mmol) in MeOH (100 mL) was slowly added NH4Cl (4.69 g, 87.7 mmol) and Fe powder (2.46 g, 43.85 mmol) at RT. The mixture was heated to 70° C. and stirred for 2 hrs. The mixture was in vacuum. The residue was purified by silica gel column chromatography (PE:EA=3:1 to 0:1) to give crude product (3.3 g, 77.8%). [M+H]⁺=484.3.

### Step 8: methyl 2-(4-(4-(tert-butoxycarbonyl)piperazin-1-yl)bicyclo[2.2.2]octan-1-yl)-5-(2,2,2-trifluoroacetamido)-2H-indazole-6-carboxylate

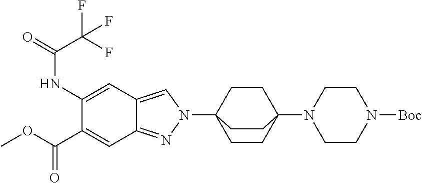 [SMILES: COC(=O)c1cc2nn(C34CCC(N5CCN(C(=O)OC(C)(C)C)CC5)(CC3)CC4)cc2cc1NC(=O)C(F)(F)F]

To a solution of methyl 5-amino-2-(4-(4-(tert-butoxycarbonyl)piperazin-1-yl)bicyclo[2.2.2]octan-1-yl)-2H-indazole-6-carboxylate (3.3 g, 6.83 mmol) in DCM (50 mL) was slowly added TFAA (2.15 g, 10.24 mmol) at RT. The mixture was stirred at rt for 2 hrs. The mixture was in vacuum. The residue was purified by silica gel column chromatography (PE:EA=3:1 to 0:1) to give crude product (3.6 g, 91%). [M+H]⁺=580.3.

### Step 9: tert-butyl 4-(4-(6-(2-hydroxypropan-2-yl)-5-(2,2,2-trifluoroacetamido)-2H-indazol-2-yl)bicyclo[2.2.2]octan-1-yl)piperazine-1-carboxylate

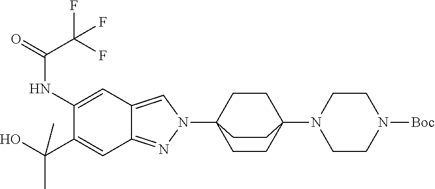 [SMILES: CC(C)(C)OC(=O)N1CCN(C23CCC(n4cc5cc(NC(=O)C(F)(F)F)c(C(C)(C)O)cc5n4)(CC2)CC3)CC1]

To a solution of methyl 2-(4-(4-(tert-butoxycarbonyl)piperazin-1-yl)bicyclo[2.2.2]octan-1-yl)-5-(2,2,2-trifluoroacetamido)-2H-indazole-6-carboxylate (3.6 g, 6.21 mmol) in dry THF (50 mL) was slowly added MeMgBr (10.4 mL, 31.07 mmol) at RT. The mixture was stirred at rt for 2 hrs. The mixture was quenched with saturated NH₄Cl and extracted with DCM. The combined organic layer was concentrated in vacuum. The residue was purified by silica gel column chromatography (PE:EA=3:1 to 0:1) to give crude product (3.0 g, 83.3%). [M+H]⁺=580.3.

### Step 10: tert-butyl 4-(4-(5-amino-6-(2-hydroxypropan-2-yl)-2H-indazol-2-yl)bicyclo[2.2.2]octan-1-yl)piperazine-1-carboxylate

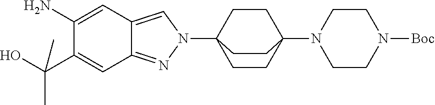 [SMILES: CC(C)(C)OC(=O)N1CCN(C23CCC(n4cc5cc(N)c(C(C)(C)O)cc5n4)(CC2)CC3)CC1]

To a solution of tert-butyl 4-(4-(6-(2-hydroxypropan-2-yl)-5-(2,2,2-trifluoroacetamido)-2H-indazol-2-yl)bicyclo[2.2.2]octan-1-yl)piperazine-1-carboxylate (3.0 g, 5.18 mmol) in MeOH (15 mL) was added aqueous NaOH (2.07 g, 51.8 mmol) in 15 mL water. The mixture was stirred at 70° C. for 3 hrs. The suspension was filtered and the solid was collected. this give product (2.0 g, 80.0%). [M+H]⁺=484.3.

### Step 11: tert-butyl 4-(4-(5-(3-cyanopyrrolo[1,2-b]pyridazine-7-carboxamido)-6-(2-hydroxypropan-2-yl)-2H-indazol-2-yl)bicyclo[2.2.2]octan-1-yl)piperazine-1-carboxylate

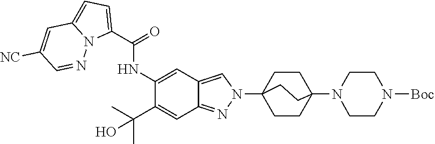 [SMILES: CC(C)(C)OC(=O)N1CCN(C23CCC(n4cc5cc(NC(=O)c6ccc7cc(C#N)cnn67)c(C(C)(C)O)cc5n4)(CC2)CC3)CC1]

To a solution of tert-butyl 4-(4-(5-amino-6-(2-hydroxypropan-2-yl)-2H-indazol-2-yl)bicyclo[2.2.2]octan-1-yl)piperazine-1-carboxylate (2.0 g, 4.13 mmol) in DMF (20 mL) was added 3-cyanopyrrolo[1,2-b]pyridazine-7-carboxylic acid (772 mg, 4313 mmol), TEA (834 mg, 8.26 mmol) and HATU (1.73 g, 4.54 mmol). The mixture was stirred at rt for 13 hrs. The suspension was poured into water (80 mL) and stirred for 10 min. The solid was collected and dried in vacuum. this give product (2.3 g, 85.0%). [M+H]⁺=653.3.

### Step 12: 3-cyano-N-(6-(2-hydroxypropan-2-yl)-2-(4-(piperazin-1-yl)bicyclo[2.2.2]octan-1-yl)-2H-indazol-5-yl)pyrrolo[1,2-b]pyridazine-7-carboxamide

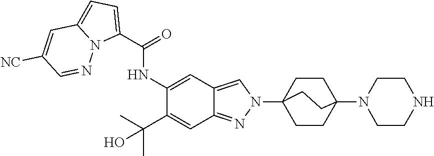 [SMILES: CC(C)(O)c1cc2nn(C34CCC(N5CCNCC5)(CC3)CC4)cc2cc1NC(=O)c1ccc2cc(C#N)cnn12]

To a solution of tert-butyl 4-(4-(5-(3-cyanopyrrolo[1,2-b]pyridazine-7-carboxamido)-6-(2-hydroxypropan-2-yl)-2H-indazol-2-yl)bicyclo[2.2.2]octan-1-yl)piperazine-1-carboxylate (2.3 g, 3.53 mmol) in DCM (10 mL) was added MeOH (10 mL) and con.HCl (10 mL). The reaction mixture was stirred at rt for 2 hrs. The solution was diluted with water (30 mL). The solution was adjusted to pH 9 with Na₂CO₃ and extracted with DCM. The combined organic layers were dried over anhydrous Na₂SO₄, filtered and evaporated in vacuum. The residue was further purification with reverse phase flash. This give product (1.3 g, 67.0%). [M+H]⁺=553.3.

### Step 13: (R)-3-cyano-N-(2-(4-(4-(1-(4-(2,6-dioxopiperidin-3-yl)-3,5-difluorophenyl)piperidin-4-yl)piperazin-1-yl)bicyclo[2.2.2]octan-1-yl)-6-(2-hydroxypropan-2-yl)-2H-indazol-5-yl)pyrrolo[1,2-b]pyridazine-7-carboxamide

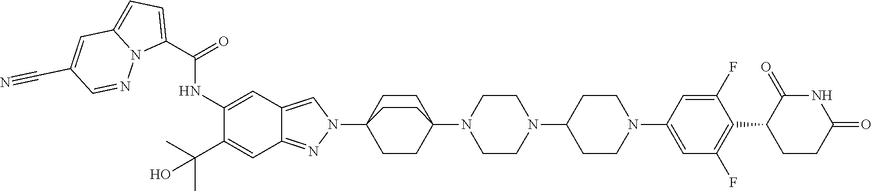 [SMILES: CC(C)(O)c1cc2nn(C34CCC(N5CCN(C6CCN(c7cc(F)c([C@H]8CCC(=O)NC8=O)c(F)c7)CC6)CC5)(CC3)CC4)cc2cc1NC(=O)c1ccc2cc(C#N)cnn12]

The title compound (18.5 mg, 23.33%) was prepared in a manner similar to that described in Example 290. ¹H NMR (500 MHz, DMSO) δ 12.02 (s, 1H), 10.87 (s, 1H), 8.93 (d, J=2.1 Hz, 1H), 8.74 (d, J=2.1 Hz, 1H), 8.55 (s, 1H), 8.34 (s, 1H), 7.71 (d, J=4.8 Hz, 1H), 7.57 (s, 1H), 7.09 (d, J=4.8 Hz, 1H), 6.63 (d, J=12.8 Hz, 2H), 5.71 (s, 1H), 4.04 (dd, J=12.5, 5.0 Hz, 1H), 3.78 (d, J=12.3 Hz, 2H), 2.82-2.70 (m, 3H), 2.60-2.52 (m, 9H), 2.40-2.38 (m, 1H), 2.24-2.15 (m, 6H), 2.13-2.03 (m, 1H), 1.98-1.93 (m, 1H), 1.86-1.80 (m, 2H), 1.80-1.71 (m, 6H), 1.63 (s, 6H), 1.42 (dd, J=20.9, 11.3 Hz, 2H). [M+H]⁺=859.6.

### Example 340: 3-cyano-N-(2-((R)-1′-(4-((R)-2,6-dioxopiperidin-3-yl)-3,5-difluorophenethyl)-3′,3′-difluoro-[1,4′-bipiperidin]-4-yl)-6-(2-hydroxypropan-2-yl)-2H-indazol-5-yl)pyrrolo[1,2-b]pyridazine-7-carboxamide

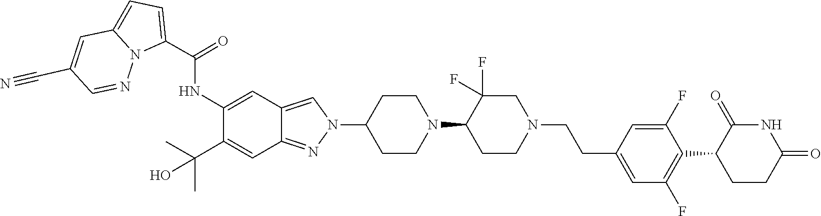 [SMILES: CC(C)(O)c1cc2nn(C3CCN([C@@H]4CCN(CCc5cc(F)c([C@H]6CCC(=O)NC6=O)c(F)c5)CC4(F)F)CC3)cc2cc1NC(=O)c1ccc2cc(C#N)cnn12]

The title compound (32 mg, 44.18%) was prepared in a manner similar to that described in Example 290. ¹H NMR (500 MHz, DMSO) δ 12.03 (s, 1H), 10.94 (s, 1H), 8.93 (d, J=2.1 Hz, 1H), 8.74 (d, J=2.1 Hz, 1H), 8.56 (s, 1H), 8.38 (s, 1H), 7.72 (d, J=4.8 Hz, 1H), 7.58 (s, 1H), 7.09 (d, J=4.8 Hz, 1H), 7.04 (d, J=10.1 Hz, 2H), 5.73 (s, 1H), 4.49-4.40 (m, 1H), 4.20 (dd, J=12.6, 4.9 Hz, 1H), 3.18-3.06 (m, 3H), 3.03-2.86 (m, 2H), 2.85-2.70 (m, 5H), 2.67-2.59 (m, 2H), 2.56-2.53 (m, 1H), 2.39-2.26 (m, 1H), 2.18-1.97 (m, 7H), 1.89-1.72 (m, 2H), 1.63 (s, 6H). [M+H]⁺=814.6.

### Example 341: 3-cyano-N-(2-((R)-1″-(4-((R)-2,6-dioxopiperidin-3-yl)-3,5-difluorophenyl)-3′,3′-difluoro-[1,4′:1′,4″-terpiperidin]-4-yl)-6-(2-hydroxypropan-2-yl)-2H-indazol-5-yl)pyrrolo[1,2-b]pyridazine-7-carboxamide

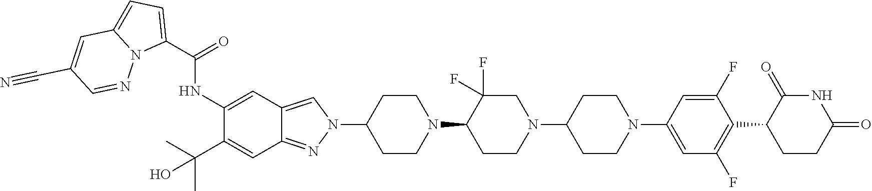 [SMILES: CC(C)(O)c1cc2nn(C3CCN([C@@H]4CCN(C5CCN(c6cc(F)c([C@H]7CCC(=O)NC7=O)c(F)c6)CC5)CC4(F)F)CC3)cc2cc1NC(=O)c1ccc2cc(C#N)cnn12]

The title compound (35 mg, 45.26%) was prepared in a manner similar to that described in Example 290. ¹H NMR (500 MHz, DMSO) δ 12.03 (s, 1H), 10.87 (s, 1H), 8.93 (d, J=2.1 Hz, 1H), 8.74 (d, J=2.0 Hz, 1H), 8.55 (s, 1H), 8.38 (s, 1H), 7.72 (d, J=4.8 Hz, 1H), 7.57 (s, 1H), 7.09 (d, J=4.8 Hz, 1H), 6.63 (d, J=12.8 Hz, 2H), 5.72 (s, 1H), 4.44 (tt, J=10.0, 5.0 Hz, 1H), 4.04 (dd, J=12.4, 5.0 Hz, 1H), 3.80 (d, J=12.1 Hz, 2H), 3.15-2.99 (m, 4H), 2.90-2.68 (m, 6H), 2.59-2.52 (m, 2H), 2.46-2.35 (m, 1H), 2.31-2.24 (m, 1H), 2.14-2.00 (m, 5H), 1.99-1.93 (m, 1H), 1.82-1.72 (m, 4H), 1.63 (s, 6H), 1.51-1.40 (m, 2H). [M+H]⁺=869.6.

### Example 342: 3-cyano-N-(2-((R)-1′-(1-(4-((R)-2,6-dioxopiperidin-3-yl)-3,5-difluorophenyl)azetidin-3-yl)-3′,3′-difluoro-[1,4′-bipiperidin]-4-yl)-6-(2-hydroxypropan-2-yl)-2H-indazol-5-yl)pyrrolo[1,2-b]pyridazine-7-carboxamide

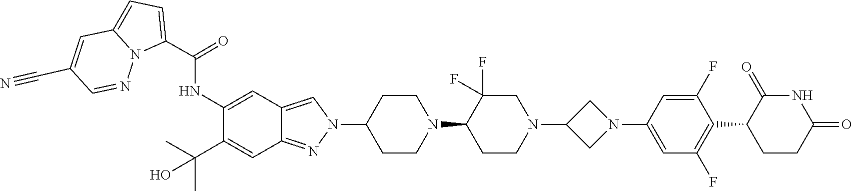 [SMILES: CC(C)(O)c1cc2nn(C3CCN([C@@H]4CCN(C5CN(c6cc(F)c([C@H]7CCC(=O)NC7=O)c(F)c6)C5)CC4(F)F)CC3)cc2cc1NC(=O)c1ccc2cc(C#N)cnn12]

The title compound (36 mg, 23.28%) was prepared in a manner similar to that described in Example 290. ¹H NMR (500 MHz, DMSO) δ 12.03 (s, 1H), 10.86 (s, 1H), 8.93 (d, J=2.2 Hz, 1H), 8.74 (d, J=2.2 Hz, 1H), 8.55 (s, 1H), 8.38 (s, 1H), 7.72 (d, J=4.8 Hz, 1H), 7.58 (s, 1H), 7.09 (d, J=4.8 Hz, 1H), 6.13 (d, J=11.1 Hz, 2H), 5.72 (s, 1H), 4.49-4.40 (m, 1H), 4.04 (dd, J=12.6, 4.9 Hz, 1H), 3.92 (dd, J=16.5, 7.8 Hz, 2H), 3.70-3.60 (m, 2H), 3.46-3.39 (m, 1H), 3.30-3.26 (m, 1H), 3.16-3.05 (m, 2H), 3.02-2.88 (m, 3H), 2.82-2.71 (m, 3H), 2.28 (dd, J=29.4, 11.3 Hz, 1H), 2.13-2.02 (m, 6H), 1.97-1.91 (m, 1H), 1.87-1.79 (m, 2H), 1.63 (s, 6H). [M+H]⁺=841.6.

### Example 183: 3-cyano-N-(2-((1S,4r)-4-((S)-4-(1-(4-((R)-2,6-dioxopiperidin-3-yl)-3,5-difluorophenyl)azetidine-3-carbonyl)-2-(methoxymethyl)piperazin-1-yl)cyclohexyl)-6-(2-hydroxypropan-2-yl)-2H-indazol-5-yl)pyrrolo[1,2-b]pyridazine-7-carboxamide

### Step 1: methyl 2-((1S,4r)-4-((S)-4-(tert-butoxycarbonyl)-2-(methoxymethyl)piperazin-1-yl)cyclohexyl)-2H-indazole-6-carboxylate

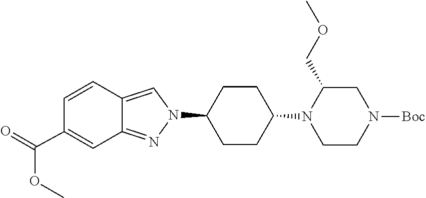 [SMILES: COC[C@@H]1CN(C(=O)OC(C)(C)C)CCN1[C@H]1CC[C@H](n2cc3ccc(C(=O)OC)cc3n2)CC1]

To a solution of methyl 4-formyl-3-nitrobenzoate (6.4 g, 30.5 mmol) in propan-2-ol (100 mL) was added tert-butyl (S)-4-((1r,4S)-4-aminocyclohexyl)-3-(methoxymethyl)piperazine-1-carboxylate (10.0 g, 30.5 mmol). The mixture was stirred for 3 hrs at 80° C. under N₂. To the above solution was added tributylphosphane (18.5 g, 91.5 mmol). The mixture was stirred overnight at 80° C. under N₂. The mixture was cooled to rt and concentrated under vacuum. The residue was purified by silica gel column chromatography (DCM:MeOH=30:1) to yield product (10.5 g, 70%). [M+H]⁺=487.3.

### Step 2: methyl 2-((1S,4r)-4-((S)-2-(methoxymethyl)piperazin-1-yl)cyclohexyl)-5-nitro-2H-indazole-6-carboxylate

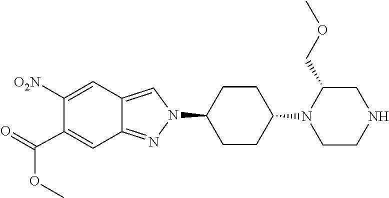 [SMILES: COC[C@@H]1CNCCN1[C@H]1CC[C@H](n2cc3cc([N+](=O)[O-])c(C(=O)OC)cc3n2)CC1]

To a solution of methyl 2-((1S,4r)-4-((S)-4-(tert-butoxycarbonyl)-2-(methoxymethyl)piperazin-1-yl)cyclohexyl)-2H-indazole-6-carboxylate (10.5 g, 21.5 mmol) in conc.H₂SO₄ (40 mL) was slowed added NaNO₃ (2.1 g, 32.3 mmol) at 0° C. The mixture was stirred for 2 hrs at 0° C. The mixture was quenched with ice-water, then adjusted to pH 9 with sat.Na₂CO₃ solution and extracted with DCM. The combined organic layer was dried over anhydrous Na₂SO₄, filtered and evaporated in vacuum to afford product (9 g, 97%), which was used without further purification. [M+H]⁺=432.2

### Step 3: methyl 2-((1S,4r)-4-((S)-4-(tert-butoxycarbonyl)-2-(methoxymethyl)piperazin-1-yl)cyclohexyl)-5-nitro-2H-indazole-6-carboxylate

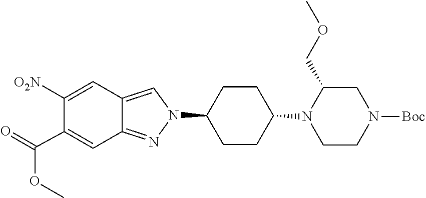 [SMILES: COC[C@@H]1CN(C(=O)OC(C)(C)C)CCN1[C@H]1CC[C@H](n2cc3cc([N+](=O)[O-])c(C(=O)OC)cc3n2)CC1]

To a solution of methyl 2-((1S,4r)-4-((S)-2-(methoxymethyl)piperazin-1-yl)cyclohexyl)-5-nitro-2H-indazole-6-carboxylate (9 g, 20.8 mmol) in DCM (100 mL) was added DIEA (5.4 g, 41.6 mmol) and Boc₂O (5.4 g, 24.96 mmol). The reaction mixture was stirred overnight at rt. The mixture was concentrated under vacuum. The residue was purified with silica gel column chromatography (DCM:MeOH=30:1) to yield product (7.7 g, 70%). [M+H]⁺=532.2.

### Step 4: methyl 5-amino-2-((1S,4r)-4-((S)-4-(tert-butoxycarbonyl)-2-(methoxymethyl)piperazin-1-yl)cyclohexyl)-2H-indazole-6-carboxylate

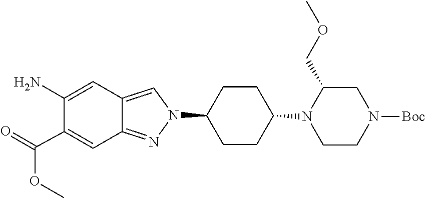 [SMILES: COC[C@@H]1CN(C(=O)OC(C)(C)C)CCN1[C@H]1CC[C@H](n2cc3cc(N)c(C(=O)OC)cc3n2)CC1]

To a solution of methyl 2-((1S,4r)-4-((S)-4-(tert-butoxycarbonyl)-2-(methoxymethyl)piperazin-1-yl)cyclohexyl)-5-nitro-2H-indazole-6-carboxylate (7.7 g, 14.5 mmol) in MeOH (100 mL) and water (20 mL) was added NH₄Cl (3.9 g, 72.5 mmol) and Fe powder (4.1 g, 72.5 mmol) at room temperature. The resulting mixture was stirred for 2 hrs at 70° C. The mixture was cooled to rt and concentrated under vacuum. The residue was purified by silica gel column chromatography (DCM:MeOH=15:1) to afford product (6.0 g, 82%). [M+H]⁺=502.3.

### Step 5: methyl 2-((1S,4r)-4-((S)-4-(tert-butoxycarbonyl)-2-(methoxymethyl)piperazin-1-yl)cyclohexyl)-5-(2,2,2-trifluoroacetamido)-2H-indazole-6-carboxylate

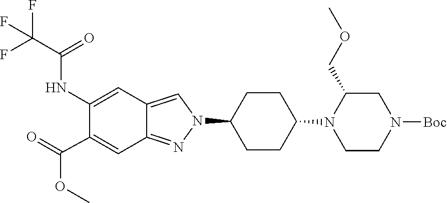 [SMILES: COC[C@@H]1CN(C(=O)OC(C)(C)C)CCN1[C@H]1CC[C@H](n2cc3cc(NC(=O)C(F)(F)F)c(C(=O)OC)cc3n2)CC1]

To a solution of methyl 5-amino-2-((1S,4r)-4-((S)-4-(tert-butoxycarbonyl)-2-(methoxymethyl)piperazin-1-yl)cyclohexyl)-2H-indazole-6-carboxylate (6.0 g, 11.95 mmol) in DCM (100 mL) was added (CF₃CO)₂O (3.0 g, 14.34 mmol). The reaction mixture was stirred overnight at rt. The mixture was concentrated under vacuum. The residue was purified with silica gel column chromatography (DCM:MeOH=30:1) to yield product (6.5 g, 91%). [M+H]⁺=598.3.

### Step 6: tert-butyl (S)-4-((1r,4S)-4-(6-(2-hydroxypropan-2-yl)-5-(2,2,2-trifluoroacetamido)-2H-indazol-2-yl)cyclohexyl)-3-(methoxymethyl)piperazine-1-carboxylate

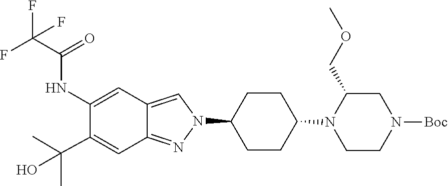 [SMILES: COC[C@@H]1CN(C(=O)OC(C)(C)C)CCN1[C@H]1CC[C@H](n2cc3cc(NC(=O)C(F)(F)F)c(C(C)(C)O)cc3n2)CC1]

To a solution of methyl 2-((1S,4r)-4-((S)-4-(tert-butoxycarbonyl)-2-(methoxymethyl)piperazin-1-yl)cyclohexyl)-5-(2,2,2-trifluoroacetamido)-2H-indazole-6-carboxylate (6.5 g, 10.87 mmol) and LiCl (1.8 g, 43.48 mmol) in THF (100 mL) was added dropwise MeMgBr in THF (3.2 M, 27 mL, 86.96 mmol) at 0° C. The resulting mixture was stirred for 2 hrs at 0° C. The mixture was quenched with sat.NH₄Cl solution and extracted with DCM. The combined organic layer was dried over anhydrous Na₂SO₄, filtered and evaporated under vacuum. The residue was purified on silica gel column chromatography (DCM:MeOH=10:1) to afford product (6 g, 92%). [M+H]⁺=598.3.

### Step 7: tert-butyl (S)-4-((1r,4S)-4-(5-amino-6-(2-hydroxypropan-2-yl)-2H-indazol-2-yl)cyclohexyl)-3-(methoxymethyl)piperazine-1-carboxylate

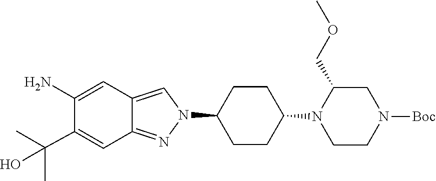 [SMILES: COC[C@@H]1CN(C(=O)OC(C)(C)C)CCN1[C@H]1CC[C@H](n2cc3cc(N)c(C(C)(C)O)cc3n2)CC1]

To a solution of tert-butyl (S)-4-((1r,4S)-4-(6-(2-hydroxypropan-2-yl)-5-(2,2,2-trifluoroacetamido)-2H-indazol-2-yl)cyclohexyl)-3-(methoxymethyl)piperazine-1-carboxylate (6.0 g, 10.0 mmol) in MeOH (50 mL) was added dropwise NaOH in H₂O (2.0 M, 50 mL, 100 mmol) at 0° C. The resulting mixture was stirred for 5 hrs at 70° C. The mixture was extracted with DCM. The combined organic layer was dried over anhydrous Na₂SO₄, filtered and evaporated under vacuum. The residue was purified on silica gel column chromatography (DCM:MeOH=10:1) to afford product (5.0 g, 99%). [M+H]⁺=502.3

### Step 8: tert-butyl (S)-4-((1r,4S)-4-(5-(3-cyanopyrrolo[1,2-b]pyridazine-7-carboxamido)-6-(2-hydroxypropan-2-yl)-2H-indazol-2-yl)cyclohexyl)-3-(methoxymethyl)piperazine-1-carboxylate

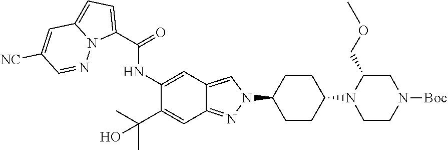 [SMILES: COC[C@@H]1CN(C(=O)OC(C)(C)C)CCN1[C@H]1CC[C@H](n2cc3cc(NC(=O)c4ccc5cc(C#N)cnn45)c(C(C)(C)O)cc3n2)CC1]

To a stirred solution of 3-cyanopyrrolo[1,2-b]pyridazine-7-carboxylic acid (744 mg, 3.98 mmol) and tert-butyl (S)-4-((1r,4S)-4-(5-amino-6-(2-hydroxypropan-2-yl)-2H-indazol-2-yl)cyclohexyl)-3-(methoxymethyl)piperazine-1-carboxylate (2.0 g, 3.98 mmol) in DMF (210 mL) was added HATU (2.3 g, 5.97 mmol) and DIEA (1.0 g, 7.96 mmol). The solution was stirred at rt for 4 h. Then the solution was diluted with EA (200 mL), washed with water and brine. The organic layer was dried over Na₂SO₄ and concentrated under vacuum. The residue was purified with Combi-Flash (silica column, 4 g, DCM:MeOH=20:1) to yield the product (2.1 g, 78%). [M+H]⁺=671.4.

### Step 9: 3-cyano-N-(6-(2-hydroxypropan-2-yl)-2-((1S,4r)-4-((S)-2-(methoxymethyl)piperazin-1-yl)cyclohexyl)-2H-indazol-5-yl)pyrrolo[1,2-b]pyridazine-7-carboxamide

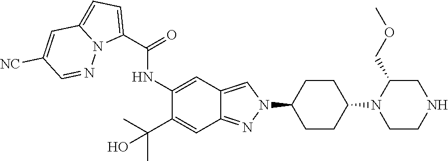 [SMILES: COC[C@@H]1CNCCN1[C@H]1CC[C@H](n2cc3cc(NC(=O)c4ccc5cc(C#N)cnn45)c(C(C)(C)O)cc3n2)CC1]

To a stirring solution of tert-butyl (S)-4-((1r,4S)-4-(5-(3-cyanopyrrolo[1,2-b]pyridazine-7-carboxamido)-6-(2-hydroxypropan-2-yl)-2H-indazol-2-yl)cyclohexyl)-3-(methoxymethyl)piperazine-1-carboxylate (2.1 g, 3.13 mmol) in DCM (20 mL) and MeOH (20 mL) was slowly dropwised con. HCl (20 mL) at 0° C. The reaction mixture was stirred at rt for 2 h at r.t. and to the mixture was added H₂O (50 ml), then adjusted to pH>8 with NaHCO₃ solid. The mixture was extracted with DCM (100 ml×6), the organic layer was dried with Na₂SO₄, filtered and concentrated to afford the crude product (1.5 g, 84%). [M+H]⁺=571.3.

### Step 10: 3-cyano-N-(2-((1S,4r)-4-((S)-4-(1-(4-((R)-2,6-dioxopiperidin-3-yl)-3,5-difluorophenyl)azetidine-3-carbonyl)-2-(methoxymethyl)piperazin-1-yl)cyclohexyl)-6-(2-hydroxypropan-2-yl)-2H-indazol-5-yl)pyrrolo[1,2-b]pyridazine-7-carboxamide

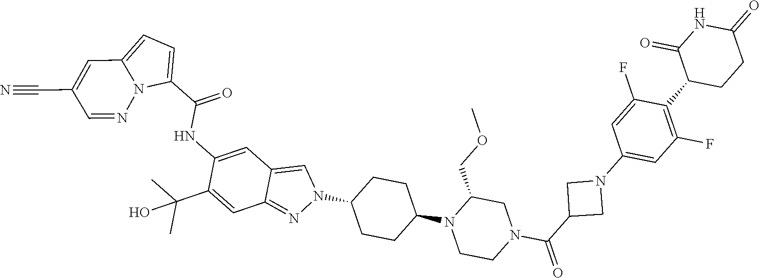 [SMILES: COC[C@@H]1CN(C(=O)C2CN(c3cc(F)c([C@H]4CCC(=O)NC4=O)c(F)c3)C2)CCN1[C@H]1CC[C@H](n2cc3cc(NC(=O)c4ccc5cc(C#N)cnn45)c(C(C)(C)O)cc3n2)CC1]

To a solution of 3-cyano-N-(6-(2-hydroxypropan-2-yl)-2-((1S,4r)-4-((S)-2-(methoxymethyl)piperazin-1-yl)cyclohexyl)-2H-indazol-5-yl)pyrrolo[1,2-b]pyridazine-7-carboxamide (100 mg, 0.175 mmol), (R)-1-(4-(2,6-dioxopiperidin-3-yl)-3,5-difluorophenyl)azetidine-3-carboxylic acid (68 mg, 0.21 mmol) and DIEA (0.5 ml) in DCM (5 mL) was added T₃P (10 drops) at room temperature. The resulting mixture was stirred at room temperature for 3 hour. The reaction was quenched with water (50 mL) and extracted with DCM (2×50 mL). The combined organic layers were dried over anhydrous Na₂SO₄, filtered and evaporated in vacuum to afford the crude residue, which was purified by silica gel column chromatography (DCM:MeOH=100:0˜10:1 gradient elution) to afford product, which was further purified with prep-HPLC (C-18 column chromatography (0.1% FA in water:acetonitrile=90:10˜60:40 gradient elution) to yield the desired product (58 mg, 37.9%).

¹H NMR (500 MHz, DMSO) δ 12.02 (s, 1H), 10.86 (s, 1H), 8.94 (d, J=5.0 Hz, 1H), 8.74 (d, J=5.0 Hz, 1H), 8.55 (s, 1H), 8.34 (s, 1H), 7.72 (d, J=5.0 Hz, 1H), 7.57 (s, 1H), 7.10 (d, J=5.0 Hz, 1H), 6.17 (d, J=10.0 Hz, 2H), 5.72 (s, 1H), 4.45-4.41 (m, 1H), 4.06-4.01 (m, 3H), 3.93-3.90 (m, 2H), 3.86-3.69 (m, 2H), 3.52-3.42 (m, 2H), 3.37-3.35 (m, 2H), 3.28 (s, 3H), 3.22-3.05 (m, 2H), 2.83-2.74 (m, 4H), 2.48-2.46 (m, 1H), 2.20-2.15 (m, 2H), 2.09-1.94 (m, 5H), 1.85-1.80 (m, 1H), 1.70-1.63 (m, 7H), 1.44-1.40 (m, 1H). [M+H]⁺=877.7.

### Example 185: 3-cyano-N-(2-((1R,4r)-4-((R)-4-(1-(4-((R)-2,6-dioxopiperidin-3-yl)-3,5-difluorophenyl)azetidine-3-carbonyl)-3-(methoxymethyl)piperazin-1-yl)cyclohexyl)-6-(2-hydroxypropan-2-yl)-2H-indazol-5-yl)pyrrolo[1,2-b]pyridazine-7-carboxamide

### Step 1: tert-butyl (R)-4-((1r,4R)-4-(((benzyloxy)carbonyl)amino)cyclohexyl)-2-(methoxymethyl)piperazine-1-carboxylate

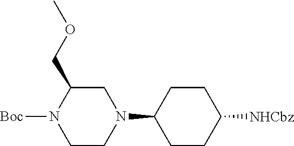 [SMILES: COC[C@H]1CN([C@H]2CC[C@H](NC(=O)OCc3ccccc3)CC2)CCN1C(=O)OC(C)(C)C]

Into a 1 L reactor, tert-butyl (R)-2-(methoxymethyl)piperazine-1-carboxylate (37.00 g, 160.658 mmol), THF (400 mL), HOAc (2 mL) and 4-N-Cbz-cyclohexanone (39.73 g, 160.658 mmol) were added. STAB (68.10 g, 321.316 mmol) was added dropwise to the resulting mixture at room temperature. The reaction mixture was stirred at room temperature for 16 hrs. Water (200 mL) was added to the mixture and pH was adjusted to 7 with saturated aq. Na₂CO₃. The mixture was extracted with EtOAc (200 mL×2) and the organic layers were washed with brine (200 mL), dried over MgSO₄, filtered and concentrated to give crude product as a light yellow oil.

The crude product was purified by prep.HPLC to give tert-butyl (R)-4-((1r,4R)-4-(((benzyloxy)carbonyl)amino)cyclohexyl)-2-(methoxymethyl)piperazine-1-carboxylate (30.00 g, 40.45%) ¹HNMR (400 MHz, CDCl₃) δ ppm: 7.38-7.28 (m, 5H), 5.10 (s, 2H), 4.62-4.56 (m, 1H), 4.20-4.14 (m, 1H), 3.88-3.84 (m, 1H), 3.72-3.67 (m, 1H), 3.44-3.32 (m, 5H), 2.94-2.89 (m, 2H), 2.73-2.67 (m, 1H), 2.28-2.23 (m, 3H), 2.11-2.07 (m, 2H), 1.87-1.80 (m, 2H), 1.50-1.10 (m, 13H).

Instrument: Shimadzu LC-20AP (QC-R-LC-09); Column: YMC Triart C18 50*250 mm 10 um, Mobile phase: A for H2O (0.1% NH₃·H₂O) and B for MEOH, Gradient: B 75% 27 min, Flow rate: 70 mL/min, Column temperature: 40° C., Wavelength: 220 nm &210 nm, Run time: 27 min, Sample preparation: Compound was dissolved in ˜800 m1 MEOH/H₂O, Injection: 24.0 ml per injection.

### Step 2: tert-butyl (R)-4-((1r,4R)-4-aminocyclohexyl)-2-(methoxymethyl)piperazine-1-carboxylate

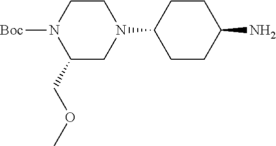 [SMILES: COC[C@H]1CN([C@H]2CC[C@H](N)CC2)CCN1C(=O)OC(C)(C)C]

To a solution of tert-butyl (R)-4-((1r,4R)-4-(((benzyloxy)carbonyl)amino)cyclohexyl)-2-(methoxymethyl)piperazine-1-carboxylate (30.00 g, 64.992 mmol) in MeOH (300 mL) was added 10% Pd/C (6.0 g). Under 1 atm hydrogen atmosphere, the mixture was stirred at room temperature for 16 hrs. The mixture was filtered and concentrated to give crude product. DCM (200 mL) was added. The solution was concentrated under reduced pressure to give tert-butyl (R)-4-((1r,4R)-4-aminocyclohexyl)-2-(methoxymethyl)piperazine-1-carboxylate (16.34 g, 76.78%). ¹HNMR (400 MHz, CDCl₃) δ ppm: 4.15 (s, 1H), 3.84-3.80 (m, 1H), 3.70-3.65 (m, 1H), 3.33-3.31 (m, 4H), 2.91-2.87 (m, 2H), 2.70-2.57 (m, 2H), 2.28-2.23 (m, 3H), 1.90-1.78 (m, 4H), 1.45 (s, 9H), 1.32-1.12 (m, 6H).

### Step 3: methyl 2-((1R,4r)-4-((R)-4-(tert-butoxycarbonyl)-3-(methoxymethyl)piperazin-1-yl)cyclohexyl)-2H-indazole-6-carboxylate

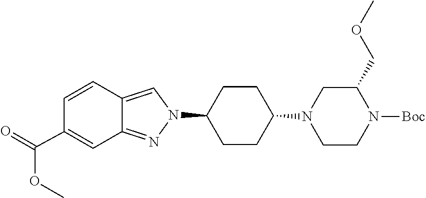 [SMILES: COC[C@H]1CN([C@H]2CC[C@H](n3cc4ccc(C(=O)OC)cc4n3)CC2)CCN1C(=O)OC(C)(C)C]

To a solution of methyl 4-formyl-3-nitrobenzoate (5.2 g, 24.4 mmol) in propan-2-ol (100 mL) was added tert-butyl (R)-4-((1r,4R)-4-aminocyclohexyl)-2-(methoxymethyl)piperazine-1-carboxylate (8.0 g, 24.4 mmol). The mixture was stirred for 3 hrs at 80° C. under N₂. To the above solution was added tributylphosphane (15.0 g, 73.2 mmol). The mixture was stirred overnight at 80° C. under N₂. The mixture was cooled to rt and concentrated under vacuum. The residue was purified by silica gel column chromatography (DCM:MeOH=30:1) to yield product (8.7 g, 74%). [M+H]⁺=487.3.

### Step 4: methyl 2-((1R,4r)-4-((R)-3-(methoxymethyl)piperazin-1-yl)cyclohexyl)-5-nitro-2H-indazole-6-carboxylate

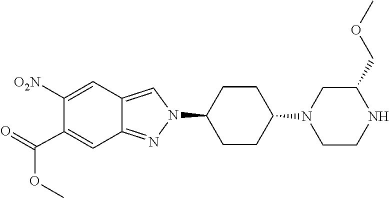 [SMILES: COC[C@H]1CN([C@H]2CC[C@H](n3cc4cc([N+](=O)[O-])c(C(=O)OC)cc4n3)CC2)CCN1]

To a solution of methyl 2-((1R,4r)-4-((R)-4-(tert-butoxycarbonyl)-3-(methoxymethyl)piperazin-1-yl)cyclohexyl)-2H-indazole-6-carboxylate (8.7 g, 17.9 mmol) in conc.H₂SO₄ (30 mL) was slowed added NaNO₃ (2.3 g, 35.8 mmol) at 0° C. The mixture was stirred for 2 hrs at 0° C. The mixture was quenched with ice-water, then adjusted to pH 9 with sat.Na₂CO₃ solution and extracted with DCM. The combined organic layer was dried over anhydrous Na₂SO₄, filtered and evaporated in vacuum to afford product (7.5 g, 97%), which was used without further purification. [M+H]⁺=432.2.

### Step 5: methyl 2-((1R,4r)-4-((R)-4-(tert-butoxycarbonyl)-3-(methoxymethyl)piperazin-1-yl)cyclohexyl)-5-nitro-2H-indazole-6-carboxylate

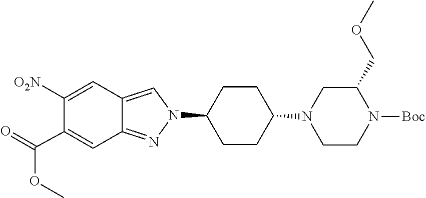 [SMILES: COC[C@H]1CN([C@H]2CC[C@H](n3cc4cc([N+](=O)[O-])c(C(=O)OC)cc4n3)CC2)CCN1C(=O)OC(C)(C)C]

To a solution of methyl 2-((1R,4r)-4-((R)-3-(methoxymethyl)piperazin-1-yl)cyclohexyl)-5-nitro-2H-indazole-6-carboxylate (7.5 g, 17.4 mmol) in DCM (100 mL) was added DIEA (4.5 g, 34.8 mmol) and Boc₂O (4.8 g, 20.9 mmol). The reaction mixture was stirred overnight at rt. The mixture was concentrated under vacuum. The residue was purified with silica gel column chromatography (DCM:MeOH=30:1) to yield product (8.5 g, 92%). [M+H]⁺=532.2.

### Step 6: methyl 5-amino-2-((1R,4r)-4-((R)-4-(tert-butoxycarbonyl)-3-(methoxymethyl)piperazin-1-yl)cyclohexyl)-2H-indazole-6-carboxylate

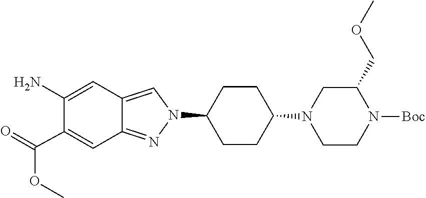 [SMILES: COC[C@H]1CN([C@H]2CC[C@H](n3cc4cc(N)c(C(=O)OC)cc4n3)CC2)CCN1C(=O)OC(C)(C)C]

To a solution of methyl 2-((1R,4r)-4-((R)-4-(tert-butoxycarbonyl)-3-(methoxymethyl)piperazin-1-yl)cyclohexyl)-5-nitro-2H-indazole-6-carboxylate (8.5 g, 16.0 mmol) in MeOH (100 mL) and water (20 mL) was added NH₄Cl (4.3 g, 80.0 mmol) and Fe powder (4.5 g, 80.0 mmol) at room temperature. The resulting mixture was stirred for 2 hrs at 70° C. The mixture was cooled to rt and concentrated under vacuum. The residue was purified by silica gel column chromatography (DCM:MeOH=15:1) to afford product (5.7 g, 71%). [M+H]⁺=502.3.

### Step 7: methyl 2-((1R,4r)-4-((R)-4-(tert-butoxycarbonyl)-3-(methoxymethyl)piperazin-1-yl)-cyclohexyl)-5-(2,2,2-trifluoroacetamido)-2H-indazole-6-carboxylate

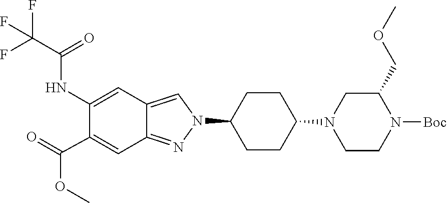 [SMILES: COC[C@H]1CN([C@H]2CC[C@H](n3cc4cc(NC(=O)C(F)(F)F)c(C(=O)OC)cc4n3)CC2)CCN1C(=O)OC(C)(C)C]

To a solution of methyl 5-amino-2-((1R,4r)-4-((R)-4-(tert-butoxycarbonyl)-3-(methoxymethyl)piperazin-1-yl)cyclohexyl)-2H-indazole-6-carboxylate (5.7 g, 11.4 mmol) in DCM (100 mL) was added (CF₃CO)₂O (2.9 g, 13.6 mmol). The reaction mixture was stirred overnight at rt. The mixture was concentrated under vacuum. The residue was purified with silica gel column chromatography (DCM:MeOH=30:1) to yield product (5.8 g, 85%). [M+H]⁺=598.3.

### Step 8: tert-butyl (R)-4-((1r,4R)-4-(6-(2-hydroxypropan-2-yl)-5-(2,2,2-trifluoroacetamido)-2H-indazol-2-yl)cyclohexyl)-2-(methoxymethyl)piperazine-1-carboxylate

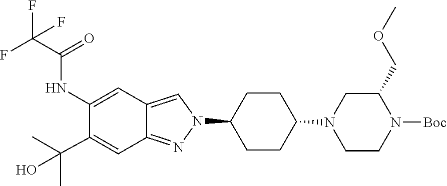 [SMILES: COC[C@H]1CN([C@H]2CC[C@H](n3cc4cc(NC(=O)C(F)(F)F)c(C(C)(C)O)cc4n3)CC2)CCN1C(=O)OC(C)(C)C]

To a solution of methyl 2-((1R,4r)-4-((R)-4-(tert-butoxycarbonyl)-3-(methoxymethyl)piperazin-1-yl)cyclohexyl)-5-(2,2,2-trifluoroacetamido)-2H-indazole-6-carboxylate (5.8 g, 9.7 mmol) and LiCl (1.63 g, 38.8 mmol) in THF (100 mL) was added dropwise MeMgBr in THF (3.2 M, 24 mL, 77.6 mmol) at 0° C. The resulting mixture was stirred for 2 hrs at 0° C. The mixture was quenched with sat.NH₄Cl solution and extracted with DCM. The combined organic layer was dried over anhydrous Na₂SO₄, filtered and evaporated under vacuum. The residue was purified on silica gel column chromatography (DCM:MeOH=10:1) to afford product (5.5 g, 95%). [M+H]⁺=598.3.

### Step 9: tert-butyl (R)-4-((1r,4R)-4-(5-amino-6-(2-hydroxypropan-2-yl)-2H-indazol-2-yl)cyclohexyl)-2-(methoxymethyl)piperazine-1-carboxylate

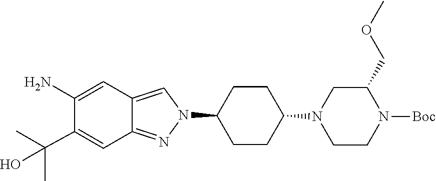 [SMILES: COC[C@H]1CN([C@H]2CC[C@H](n3cc4cc(N)c(C(C)(C)O)cc4n3)CC2)CCN1C(=O)OC(C)(C)C]

To a solution of tert-butyl (R)-4-((1r,4R)-4-(6-(2-hydroxypropan-2-yl)-5-(2,2,2-trifluoroacetamido)-2H-indazol-2-yl)cyclohexyl)-2-(methoxymethyl)piperazine-1-carboxylate (5.5 g, 9.2 mmol) in MeOH (50 mL) was added dropwise NaOH in H₂O (2.0 M, 46 mL, 92 mmol) at 0° C. The resulting mixture was stirred for 5 hrs at 70° C. The mixture was extracted with DCM. The combined organic layer was dried over anhydrous Na₂SO₄, filtered and evaporated under vacuum. The residue was purified on silica gel column chromatography (DCM:MeOH=10:1) to afford product (4.5 g, 98%). [M+H]⁺=502.3.

### Step 10: tert-butyl (R)-4-((1r,4R)-4-(5-(3-cyanopyrrolo[1,2-b]pyridazine-7-carboxamido)-6-(2-hydroxypropan-2-yl)-2H-indazol-2-yl)cyclohexyl)-2-(methoxymethyl)piperazine-1-carboxylate

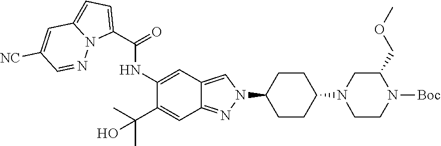 [SMILES: COC[C@H]1CN([C@H]2CC[C@H](n3cc4cc(NC(=O)c5ccc6cc(C#N)cnn56)c(C(C)(C)O)cc4n3)CC2)CCN1C(=O)OC(C)(C)C]

To a stirred solution of 3-cyanopyrrolo[1,2-b]pyridazine-7-carboxylic acid (744 mg, 3.98 mmol) and tert-butyl (R)-4-((1r,4R)-4-(5-amino-6-(2-hydroxypropan-2-yl)-2H-indazol-2-yl)cyclohexyl)-2-(methoxymethyl)piperazine-1-carboxylate (2.0 g, 3.98 mmol) in DMF (210 mL) was added HATU (2.3 g, 5.97 mmol) and DIEA (1.0 g, 7.96 mmol). The solution was stirred at rt for 4 h. Then the solution was diluted with EA (200 mL), washed with water and brine. The organic layer was dried over Na₂SO₄ and concentrated under vacuum. The residue was purified with Combi-Flash (silica column, 4 g, DCM:MeOH=20:1) to yield the product (2.0 g, 78%). [M+H]⁺=671.4.

### Step 11: 3-cyano-N-(6-(2-hydroxypropan-2-yl)-2-((1R,4r)-4-((R)-3-(methoxymethyl)piperazin-1-yl)cyclohexyl)-2H-indazol-5-yl)pyrrolo[1,2-b]pyridazine-7-carboxamide

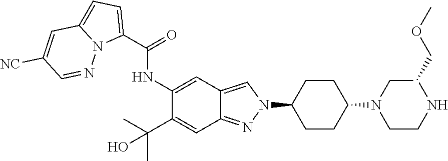

To a stirring solution of tert-butyl (R)-4-((1r,4R)-4-(5-(3-cyanopyrrolo[1,2-b]pyridazine-7-carboxamido)-6-(2-hydroxypropan-2-yl)-2H-indazol-2-yl)cyclohexyl)-2-(methoxymethyl)piperazine-1-carboxylate (2 g, 2.98 mmol) in DCM (20 mL) and MeOH (20 mL) was slowly dropwise con. HCl (20 mL) at 0° C. The reaction mixture was stirred at rt for 2 h at r.t. and to the mixture was added H₂O (50 ml), then adjusted the pH>8 with NaHCO₃ solid. The mixture was extracted with DCM (100 ml×6), the organic layer was dried with Na₂SO₄, filtered and concentrated to afford the crude product (1.5 g, 88%). [M+H]⁺=571.3.

### Step 12: 3-cyano-N-(2-((1R,4r)-4-((R)-4-(1-(4-((R)-2,6-dioxopiperidin-3-yl)-3,5-difluorophenyl)azetidine-3-carbonyl)-3-(methoxymethyl)piperazin-1-yl)cyclohexyl)-6-(2-hydroxypropan-2-yl)-2H-indazol-5-yl)pyrrolo[1,2-b]pyridazine-7-carboxamide

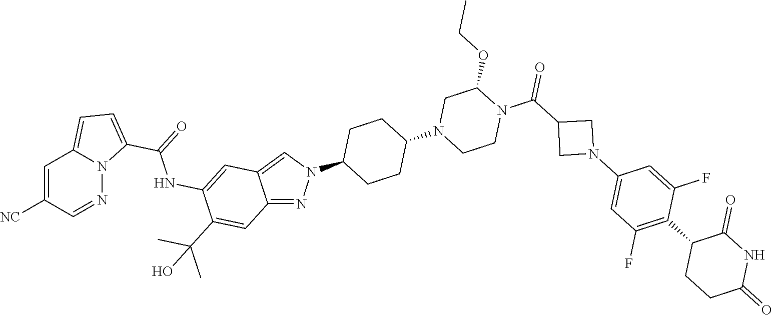 [SMILES: CCO[C@H]1CN([C@H]2CC[C@H](n3cc4cc(NC(=O)c5ccc6cc(C#N)cnn56)c(C(C)(C)O)cc4n3)CC2)CCN1C(=O)C1CN(c2cc(F)c([C@H]3CCC(=O)NC3=O)c(F)c2)C1]

To a solution of 3-cyano-N-(6-(2-hydroxypropan-2-yl)-2-((1R,4r)-4-((R)-3-(methoxymethyl)piperazin-1-yl)cyclohexyl)-2H-indazol-5-yl)pyrrolo[1,2-b]pyridazine-7-carboxamide (50 mg, 0.0876 mmol), (R)-1-(4-(2,6-dioxopiperidin-3-yl)-3,5-difluorophenyl)azetidine-3-carboxylic acid (34 mg, 0.1051 mmol) and DIEA (0.5 ml) in DCM (5 mL) was added T₃P (7 drops) at room temperature. The resulting mixture was stirred at room temperature for 3 hours. The reaction was quenched with water (20 mL) and extracted with DCM (2×10 mL). The combined organic layers were dried over anhydrous Na₂SO₄, filtered and evaporated in vacuum to afford the crude residue, which was purified by silica gel column chromatography (DCM:MeOH=100:0˜10:1 gradient elution) to afford product, which was further purified with prep-HPLC (C-18 column chromatography (0.1% FA in water:acetonitrile=90:10˜60:40 gradient elution) to yield the desired product (36 mg, 47%). ¹H NMR (500 MHz, DMSO) δ 12.03 (s, 1H), 10.86 (s, 1H), 8.93 (d, J=5.0 Hz, 1H), 8.74 (d, J=5.0 Hz, 1H), 8.55 (s, 1H), 8.33 (s, 1H), 7.72 (d, J=5.0 Hz, 1H), 7.57 (s, 1H), 7.10 (d, J=5.0 Hz, 1H), 6.17 (d, J=10.0 Hz, 2H), 5.73 (s, 1H), 4.56-4.22 (m, 2H), 4.06-4.03 (m, 2H), 3.94-3.84 (m, 4H), 3.70-3.59 (m, 1H), 3.52-3.38 (m, 2H), 3.26 (s, 3H), 3.12-2.74 (m, 4H), 2.09-2.06 (m, 1H), 1.96-1.88 (m, 5H), 1.63 (s, 6H), 1.50-1.43 (m, 2H). [M+H]⁺=877.7.

### Example 199: 3-cyano-N-(2-((1r,4r)-4-(4-(1-(4-((R)-2,6-dioxopiperidin-3-yl)-3,5-difluorophenyl)azetidine-3-carbonyl)-2-oxopiperazin-1-yl)cyclohexyl)-6-(2-hydroxypropan-2-yl)-2H-indazol-5-yl)pyrrolo[1,2-b]pyridazine-7-carboxamide

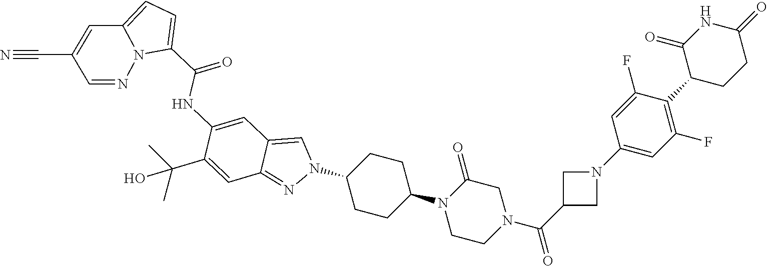 [SMILES: CC(C)(O)c1cc2nn([C@H]3CC[C@H](N4CCN(C(=O)C5CN(c6cc(F)c([C@H]7CCC(=O)NC7=O)c(F)c6)C5)CC4=O)CC3)cc2cc1NC(=O)c1ccc2cc(C#N)cnn12]

The title compound (30 mg, 35%) was prepared in a manner similar to that described in Example 185. ¹H NMR (500 MHz, DMSO) δ 12.03 (s, 1H), 10.86 (s, 1H), 8.94 (d, J=5.0 Hz, 1H), 8.74 (d, J=5.0 Hz, 1H), 8.55 (s, 1H), 8.38 (s, 1H), 7.72 (d, J=5.0 Hz, 1H), 7.58 (s, 1H), 7.10 (d, J=5.0 Hz, 1H), 6.17 (d, J=10.0 Hz, 2H), 5.73 (s, 1H), 4.49-4.39 (m, 2H), 4.05-4.03 (m, 5H), 3.95-3.88 (m, 3H), 3.69-3.68 (m, 2H), 3.59-3.58 (m, 1H), 3.39-3.32 (m, 2H), 2.81-2.74 (m, 1H), 2.22-2.20 (m, 2H), 2.07-2.02 (m, 3H), 1.96-1.93 (m, 1H), 1.86-1.75 (m, 4H), 1.64 (s, 6H). [M+H]⁺=847.5.

### Example 265: 3-cyano-N-(2-((1r,4r)-4-(4-(1-(4-(2,4-dioxotetrahydropyrimidin-1(2H)-yl)phenyl)azetidine-3-carbonyl)piperazin-1-yl)cyclohexyl)-6-(2-hydroxypropan-2-yl)-2H-indazol-5-yl)pyrrolo[1,2-b]pyridazine-7-carboxamide

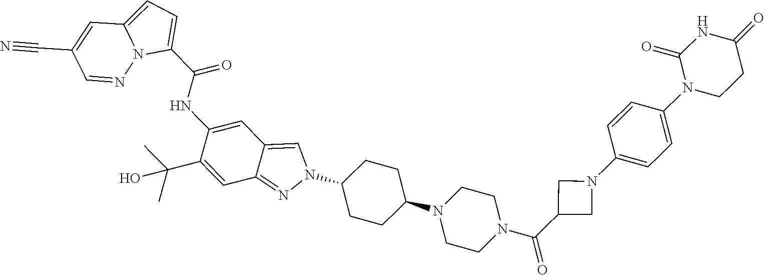 [SMILES: CC(C)(O)c1cc2nn([C@H]3CC[C@H](N4CCN(C(=O)C5CN(c6ccc(N7CCC(=O)NC7=O)cc6)C5)CC4)CC3)cc2cc1NC(=O)c1ccc2cc(C#N)cnn12]

The title compound (18 mg, 24%) was prepared in a manner similar to that described in Example 178. ¹H NMR (500 MHz, DMSO) δ 12.03 (s, 1H), 10.23 (s, 1H), 8.93 (d, J=5.0 Hz, 1H), 8.73 (d, J=5.0 Hz, 1H), 8.55 (s, 1H), 8.34 (s, 1H), 7.72 (d, J=5.0 Hz, 1H), 7.57 (s, 1H), 7.12-7.09 (m, 3H), 6.45 (d, J=10.0 Hz, 2H), 5.72 (s, 1H), 4.56-4.41 (m, 1H), 4.03-4.01 (m, 2H), 3.87-3.82 (m, 4H), 3.68-3.66 (m, 3H), 3.49-3.47 (m, 2H), 2.67-2.63 (m, 3H), 2.55-2.54 (m, 3H), 2.19-2.17 (m, 3H), 1.96-1.94 (m, 4H), 1.63 (s, 6H), 1.51-1.49 (m, 2H). [M+H]⁺=798.6.

### Example 273: 3-cyano-N-(2-((1R,4r)-4-(4-((1R,3S)-3-(4-(((S)-2,6-dioxopiperidin-3-yl)amino)phenyl)cyclobutyl)piperazin-1-yl)cyclohexyl)-6-(2-hydroxypropan-2-yl)-2H-indazol-5-yl)pyrrolo[12-b]pyridazine-7-carboxamide

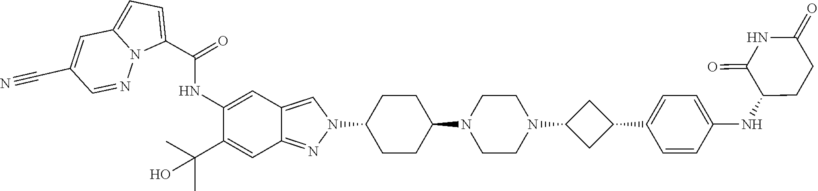 [SMILES: CC(C)(O)c1cc2nn([C@H]3CC[C@H](N4CCN([C@H]5C[C@@H](c6ccc(N[C@H]7CCC(=O)NC7=O)cc6)C5)CC4)CC3)cc2cc1NC(=O)c1ccc2cc(C#N)cnn12]

The title compound (52 mg, 17%) was prepared in a manner similar to that described in Example 290. ¹H NMR (500 MHz, DMSO) δ 12.02 (s, 1H), 10.76 (s, 1H), 8.94 (d, J=5.0 Hz, 1H), 8.74 (d, J=5.0 Hz, 1H), 8.55 (s, 1H), 8.33 (s, 1H), 7.72 (d, J=5.0 Hz, 1H), 7.57 (s, 1H), 7.10 (d, J=5.0 Hz, 1H), 6.95 (d, J=10.0 Hz, 2H), 6.61 (d, J=10.0 Hz, 2H), 5.72 (s, 1H), 5.67 (d, J=5.0 Hz, 1H), 4.43-4.40 (m, 1H), 4.30-4.25 (m, 1H), 2.96-2.93 (m, 1H), 2.78-2.71 (m, 1H), 2.64-2.55 (m, 7H), 2.43-2.25 (m, 6H), 2.18-2.08 (m, 3H), 1.97-1.84 (m, 5H), 1.76-1.73 (m, 2H), 1.63 (s, 6H), 1.51-1.47 (m, 2H). [M+H]+=783.6.

### Example 274: 3-cyano-N-(2-((1R,4r)-4-(4-((1S,3R)-3-(4-(((S)-2,6-dioxopiperidin-3-yl)amino)phenyl)cyclobutyl)piperazin-1-yl)cyclohexyl)-6-(2-hydroxypropan-2-yl)-2H-indazol-5-yl)pyrrolo[1,2-b]pyridazine-7-carboxamide

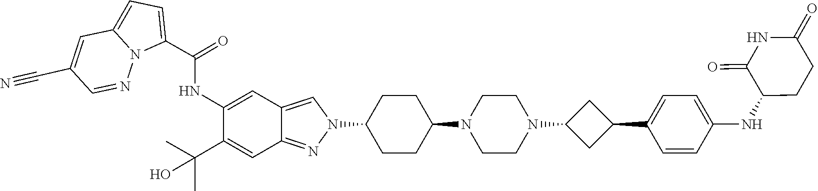 [SMILES: CC(C)(O)c1cc2nn([C@H]3CC[C@H](N4CCN([C@H]5C[C@H](c6ccc(N[C@H]7CCC(=O)NC7=O)cc6)C5)CC4)CC3)cc2cc1NC(=O)c1ccc2cc(C#N)cnn12]

The title compound (13 mg, 4.4%) was prepared in a manner similar to that described in Example 290. ¹H NMR (500 MHz, DMSO) δ 12.02 (s, 1H), 10.77 (s, 1H), 8.94 (d, J=5.0 Hz, 1H), 8.74 (d, J=5.0 Hz, 1H), 8.55 (s, 1H), 8.33 (s, 1H), 7.72 (d, J=5.0 Hz, 1H), 7.57 (s, 1H), 7.10 (d, J=5.0 Hz, 1H), 7.01 (d, J=10.0 Hz, 2H), 6.63 (d, J=10.0 Hz, 2H), 5.72 (s, 1H), 5.67 (d, J=5.0 Hz, 1H), 4.43-4.40 (m, 1H), 4.30-4.25 (m, 1H), 3.29-3.27 (m, 1H), 2.82-2.71 (m, 2H), 2.64-2.55 (m, 5H), 2.43-2.23 (m, 6H), 2.18-2.02 (m, 6H), 1.96-1.84 (m, 5H), 1.63 (s, 6H), 1.51-1.47 (m, 2H). [M+H]⁺=783.6.

### Example 271: 3-cyano-N-(2-((1R,4r)-4-(4-((1S,3S)-3-(4-(((R)-2,6-dioxopiperidin-3-yl)amino)phenyl)cyclobutyl)piperazin-1-yl)cyclohexyl)-6-(2-hydroxypropan-2-yl)-2H-indazol-5-yl)pyrrolo[1,2-b]pyridazine-7-carboxamide

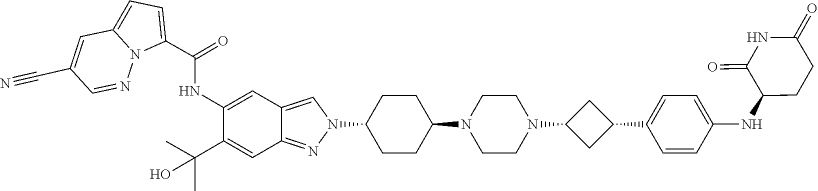 [SMILES: CC(C)(O)c1cc2nn([C@H]3CC[C@H](N4CCN([C@H]5C[C@@H](c6ccc(N[C@@H]7CCC(=O)NC7=O)cc6)C5)CC4)CC3)cc2cc1NC(=O)c1ccc2cc(C#N)cnn12]

The title compound (17 mg, 5.7%) was prepared in a manner similar to that described in Example 290. ¹H NMR (500 MHz, DMSO) δ 12.02 (s, 1H), 10.76 (s, 1H), 8.94 (d, J=5.0 Hz, 1H), 8.74 (d, J=5.0 Hz, 1H), 8.55 (s, 1H), 8.33 (s, 1H), 7.72 (d, J=5.0 Hz, 1H), 7.57 (s, 1H), 7.10 (d, J=5.0 Hz, 1H), 6.95 (d, J=10.0 Hz, 2H), 6.61 (d, J=10.0 Hz, 2H), 5.72 (s, 1H), 5.67 (d, J=5.0 Hz, 1H), 4.43-4.40 (m, 1H), 4.30-4.25 (m, 1H), 2.96-2.93 (m, 1H), 2.78-2.71 (m, 1H), 2.64-2.55 (m, 7H), 2.43-2.25 (m, 6H), 2.18-2.08 (m, 3H), 1.97-1.84 (m, 5H), 1.76-1.73 (m, 2H), 1.63 (s, 6H), 1.51-1.47 (m, 2H). [M+H]+=783.6.

### Example 272: 3-cyano-N-(2-((1R,4r)-4-(4-((1R,3R)-3-(4-(((R)-2,6-dioxopiperidin-3-yl)amino)phenyl)cyclobutyl)piperazin-1-yl)cyclohexyl)-6-(2-hydroxypropan-2-yl)-2H-indazol-5-yl)pyrrolo[1,2-b]pyridazine-7-carboxamide

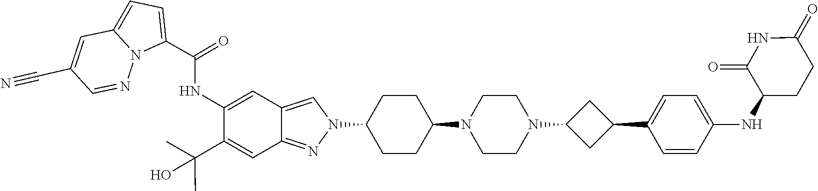 [SMILES: CC(C)(O)c1cc2nn([C@H]3CC[C@H](N4CCN([C@H]5C[C@H](c6ccc(N[C@@H]7CCC(=O)NC7=O)cc6)C5)CC4)CC3)cc2cc1NC(=O)c1ccc2cc(C#N)cnn12]

The title compound (4 mg, 1.3%) was prepared in a manner similar to that described in Example 290. ¹H NMR (500 MHz, DMSO) δ 12.02 (s, 1H), 10.77 (s, 1H), 8.94 (d, J=5.0 Hz, 1H), 8.74 (d, J=5.0 Hz, 1H), 8.55 (s, 1H), 8.33 (s, 1H), 7.72 (d, J=5.0 Hz, 1H), 7.57 (s, 1H), 7.10 (d, J=5.0 Hz, 1H), 7.01 (d, J=10.0 Hz, 2H), 6.63 (d, J=10.0 Hz, 2H), 5.72 (s, 1H), 4.43-4.40 (m, 1H), 4.30-4.25 (m, 1H), 3.29-3.27 (m, 1H), 2.82-2.71 (m, 2H), 2.64-2.55 (m, 5H), 2.43-2.23 (m, 6H), 2.18-2.02 (m, 6H), 1.96-1.84 (m, 5H), 1.63 (s, 6H), 1.51-1.47 (m, 2H). [M+H]⁺=783.6.

### Example 296: 3-cyano-N-(2-((1R,4r)-4-(4-((1s,3S)-3-(4-(2,6-dioxopiperidin-3-yl)-5-fluoro-2-methylphenyl)cyclobutyl)piperazin-1-yl)cyclohexyl)-6-(2-hydroxypropan-2-yl)-2H-indazol-5-yl)pyrrolo[1,2-b]pyridazine-7-carboxamide

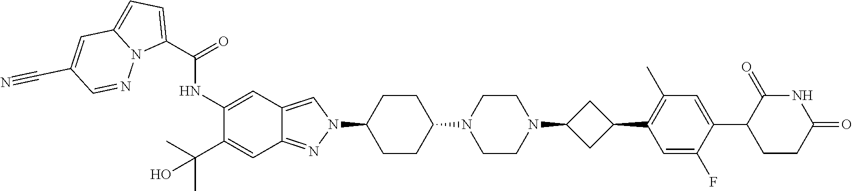 [SMILES: Cc1cc(C2CCC(=O)NC2=O)c(F)cc1[C@H]1C[C@@H](N2CCN([C@H]3CC[C@H](n4cc5cc(NC(=O)c6ccc7cc(C#N)cnn67)c(C(C)(C)O)cc5n4)CC3)CC2)C1]

The title compound (34 mg, 76%) was prepared in a manner similar to that described in Example 290. ¹H NMR (500 MHz, DMSO) δ 12.02 (s, 1H), 10.85 (s, 1H), 8.93 (d, J=5.0 Hz, 1H), 8.73 (d, J=5.0 Hz, 1H), 8.55 (s, 1H), 8.33 (s, 1H), 7.72 (d, J=5.0 Hz, 1H), 7.57 (s, 1H), 7.10 (d, J=5.0 Hz, 1H), 7.01 (d, J=10.0 Hz, 1H), 6.96 (d, J=10.0 Hz, 1H), 5.72 (s, 1H), 4.43-4.40 (m, 1H), 4.00-3.93 (m, 1H), 3.20-3.17 (m, 3H), 2.77-2.71 (m, 3H), 2.54-2.33 (m, 9H), 2.20-2.12 (m, 6H), 1.97-1.90 (m, 5H), 1.83-1.79 (m, 2H), 1.63 (s, 6H), 1.51-1.47 (m, 2H). [M+H]⁺=800.7.

### Example 297: 3-cyano-N-(2-((1R,4r)-4-(4-((1r,3R)-3-(4-(2,6-dioxopiperidin-3-yl)-5-fluoro-2-methylphenyl)cyclobutyl)piperazin-1-yl)cyclohexyl)-6-(2-hydroxypropan-2-yl)-2H-indazol-5-yl)pyrrolo[1,2-b]pyridazine-7-carboxamide

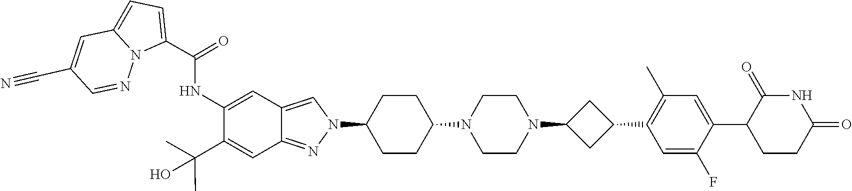 [SMILES: Cc1cc(C2CCC(=O)NC2=O)c(F)cc1[C@H]1C[C@H](N2CCN([C@H]3CC[C@H](n4cc5cc(NC(=O)c6ccc7cc(C#N)cnn67)c(C(C)(C)O)cc5n4)CC3)CC2)C1]

The title compound (6 mg, 8%) was prepared in a manner similar to that described in Example 296. ¹H NMR (500 MHz, DMSO) δ 12.03 (s, 1H), 10.86 (s, 1H), 8.93 (d, J=5.0 Hz, 1H), 8.73 (d, J=5.0 Hz, 1H), 8.55 (s, 1H), 8.34 (s, 1H), 7.72 (d, J=5.0 Hz, 1H), 7.57 (s, 1H), 7.13 (d, J=10.0 Hz, 1H), 7.10 (d, J=5.0 Hz, 1H), 7.06 (d, J=10.0 Hz, 1H), 5.72 (s, 1H), 4.45-4.40 (m, 1H), 3.97-3.94 (m, 1H), 3.50-3.48 (m, 3H), 2.80-2.70 (m, 2H), 2.58-2.55 (m, 4H), 2.41-2.31 (m, 6H), 2.22-2.12 (m, 8H), 1.99-1.91 (m, 5H), 1.63 (s, 6H), 1.52-1.45 (m, 2H). [M+H]⁺=800.7.

### Example 298: 3-cyano-N-(2-((1R,4r)-4-((R)-4-(1-(4-((R)-2,6-dioxopiperidin-3-yl)-3,5-difluorophenyl)azetidine-3-carbonyl)-2-methylpiperazin-1-yl)cyclohexyl)-6-(2-hydroxypropan-2-yl)-2H-indazol-5-yl)pyrrolo[1,2-b]pyridazine-7-carboxamide

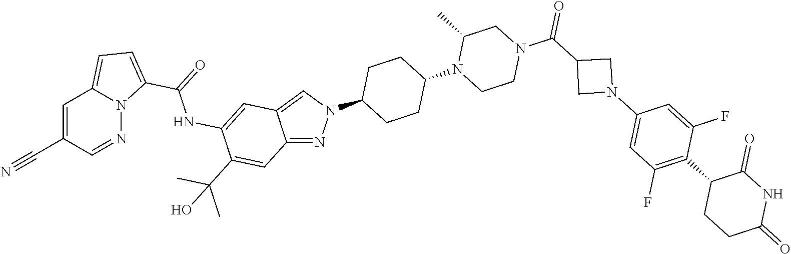 [SMILES: C[C@@H]1CN(C(=O)C2CN(c3cc(F)c([C@H]4CCC(=O)NC4=O)c(F)c3)C2)CCN1[C@H]1CC[C@H](n2cc3cc(NC(=O)c4ccc5cc(C#N)cnn45)c(C(C)(C)O)cc3n2)CC1]

The title compound (174 mg, 21%) was prepared in a manner similar to that described in Example 185. ¹H NMR (500 MHz, DMSO) δ 12.02 (s, 1H), 10.86 (s, 1H), 8.94 (d, J=5.0 Hz, 1H), 8.74 (d, J=5.0 Hz, 1H), 8.55 (s, 1H), 8.33 (s, 1H), 7.72 (d, J=5.0 Hz, 1H), 7.57 (s, 1H), 7.10 (d, J=5.0 Hz, 1H), 6.17 (d, J=10.0 Hz, 2H), 5.72 (s, 1H), 4.45-4.40 (m, 1H), 4.03-3.83 (m, 7H), 3.48-3.42 (m, 1H), 3.15-2.69 (m, 5H), 2.65-2.63 (m, 1H), 2.36-2.26 (m, 1H), 2.20-2.16 (m, 2H), 2.09-1.87 (m, 5H), 1.76-1.72 (m, 2H), 1.63 (s, 6H), 1.43-1.35 (m, 2H), 1.07-1.03 (m, 3H). [M+H]⁺=847.7.

### Example 299: 3-cyano-N-(2-((1R,4r)-4-((R)-4-(4-((R)-2,6-dioxopiperidin-3-yl)-3,5-difluorophenethyl)-2-methylpiperazin-1-yl)cyclohexyl)-6-(2-hydroxypropan-2-yl)-2H-indazol-5-yl)pyrrolo[1,2-b]pyridazine-7-carboxamide

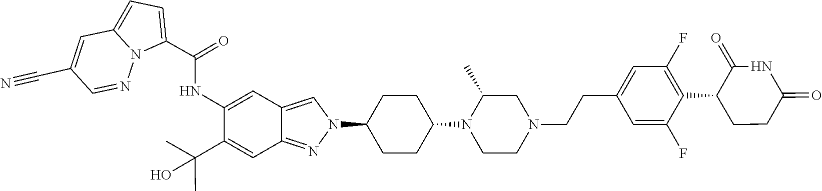 [SMILES: C[C@@H]1CN(CCc2cc(F)c([C@H]3CCC(=O)NC3=O)c(F)c2)CCN1[C@H]1CC[C@H](n2cc3cc(NC(=O)c4ccc5cc(C#N)cnn45)c(C(C)(C)O)cc3n2)CC1]

The title compound (31 mg, 39%) was prepared in a manner similar to that described in Example 314. ¹H NMR (500 MHz, DMSO) δ 12.03 (s, 1H), 10.95 (s, 1H), 8.93 (d, J=5.0 Hz, 1H), 8.74 (d, J=5.0 Hz, 1H), 8.55 (s, 1H), 8.33 (s, 1H), 7.73 (d, J=5.0 Hz, 1H), 7.57 (s, 1H), 7.10 (d, J=5.0 Hz, 1H), 7.02 (d, J=10.0 Hz, 2H), 5.72 (s, 1H), 4.44-4.39 (m, 1H), 4.22-4.18 (m, 1H), 2.91-2.72 (m, 8H), 2.55-2.54 (m, 2H), 2.46-2.42 (m, 1H), 2.20-2.07 (m, 4H), 1.99-1.89 (m, 5H), 1.74-1.70 (m, 2H), 1.63 (s, 6H), 1.44-1.38 (m, 2H), 1.04 (d, J=5.0 Hz, 3H). [M+H]⁺=792.8.

### Example 300: 3-cyano-N-(2-((1R,4r)-4-((R)-4-((1-(4-((R)-2,6-dioxopiperidin-3-yl)-3,5-difluorophenyl)azetidin-3-yl)methyl)-2-methylpiperazin-1-yl)cyclohexyl)-6-(2-hydroxypropan-2-yl)-2H-indazol-5-yl)pyrrolo[1,2-b]pyridazine-7-carboxamide

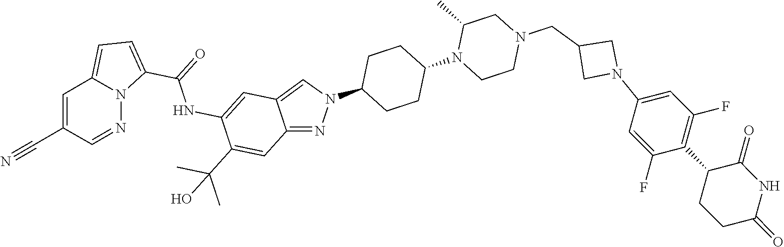 [SMILES: C[C@@H]1CN(CC2CN(c3cc(F)c([C@H]4CCC(=O)NC4=O)c(F)c3)C2)CCN1[C@H]1CC[C@H](n2cc3cc(NC(=O)c4ccc5cc(C#N)cnn45)c(C(C)(C)O)cc3n2)CC1]

The title compound (41 mg, 49%) was prepared in a manner similar to that described in Example 314. ¹H NMR (500 MHz, DMSO) δ 12.02 (s, 1H), 10.85 (s, 1H), 8.94 (d, J=5.0 Hz, 1H), 8.74 (d, J=5.0 Hz, 1H), 8.55 (s, 1H), 8.33 (s, 1H), 7.72 (d, J=5.0 Hz, 1H), 7.57 (s, 1H), 7.10 (d, J=5.0 Hz, 1H), 6.11 (d, J=10.0 Hz, 2H), 5.72 (s, 1H), 4.44-4.39 (m, 1H), 4.05-4.01 (m, 1H), 3.94-3.91 (m, 2H), 3.48-3.45 (m, 2H), 2.93-2.89 (m, 2H), 2.80-2.62 (m, 5H), 2.54-2.49 (m, 2H), 2.41-2.36 (m, 1H), 2.18-2.16 (m, 2H), 2.10-2.01 (m, 3H), 1.98-1.84 (m, 4H), 1.76-1.71 (m, 2H), 1.63 (s, 6H), 1.43-1.35 (m, 2H), 1.02 (d, J=5.0 Hz, 3H). [M+H]⁺=833.7.

### Example 301: 3-cyano-N-(2-((1R,4r)-4-((R)-4-(1-(4-((R)-2,6-dioxopiperidin-3-yl)-3,5-difluorophenyl)piperidin-4-yl)-2-methylpiperazin-1-yl)cyclohexyl)-6-(2-hydroxypropan-2-yl)-2H-indazol-5-yl)pyrrolo[1,2-b]pyridazine-7-carboxamide

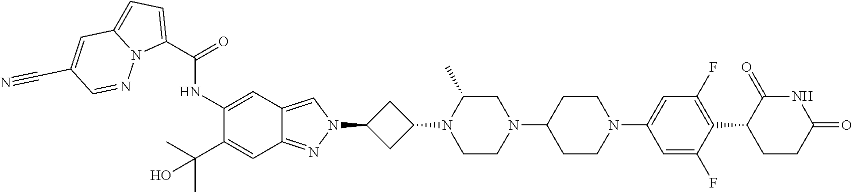 [SMILES: C[C@@H]1CN(C2CCN(c3cc(F)c([C@H]4CCC(=O)NC4=O)c(F)c3)CC2)CCN1[C@H]1C[C@H](n2cc3cc(NC(=O)c4ccc5cc(C#N)cnn45)c(C(C)(C)O)cc3n2)C1]

The title compound (9 mg, 11%) was prepared in a manner similar to that described in Example 314. ¹H NMR (500 MHz, DMSO) δ 12.02 (s, 1H), 10.87 (s, 1H), 8.93 (d, J=5.0 Hz, 1H), 8.74 (d, J=5.0 Hz, 1H), 8.55 (s, 1H), 8.33 (s, 1H), 7.72 (d, J=5.0 Hz, 1H), 7.56 (s, 1H), 7.10 (d, J=5.0 Hz, 1H), 6.64 (d, J=10.0 Hz, 2H), 5.73 (s, 1H), 4.44-4.39 (m, 1H), 4.05-4.02 (m, 1H), 3.81-3.79 (m, 2H), 2.89-2.75 (m, 8H), 2.54-2.53 (m, 1H), 2.40-2.30 (m, 2H), 2.25-1.87 (m, 9H), 1.82-1.72 (m, 4H), 1.63 (s, 6H), 1.43-1.35 (m, 3H), 1.02 (d, J=5.0 Hz, 3H). [M+H]⁺=847.8.

### Example 302: 3-cyano-N-(2-((1R,4r)-4-((R)-4-(1-(4-((R)-2,6-dioxopiperidin-3-yl)-3,5-difluorophenyl)azetidin-3-yl)-2-methylpiperazin-1-yl)cyclohexyl)-6-(2-hydroxypropan-2-yl)-2H-indazol-5-yl)pyrrolo[1,2-b]pyridazine-7-carboxamide

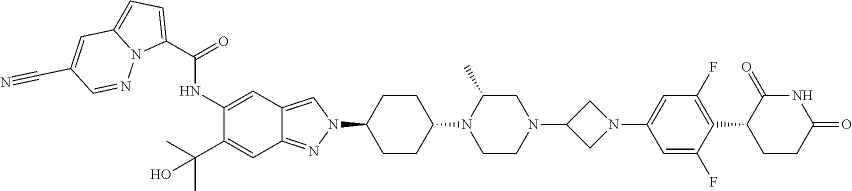 [SMILES: C[C@@H]1CN(C2CN(c3cc(F)c([C@H]4CCC(=O)NC4=O)c(F)c3)C2)CCN1[C@H]1CC[C@H](n2cc3cc(NC(=O)c4ccc5cc(C#N)cnn45)c(C(C)(C)O)cc3n2)CC1]

The title compound (12 mg, 15%) was prepared in a manner similar to that described in Example 314. ¹H NMR (500 MHz, DMSO) δ 12.02 (s, 1H), 10.86 (s, 1H), 8.94 (d, J=5.0 Hz, 1H), 8.74 (d, J=5.0 Hz, 1H), 8.55 (s, 1H), 8.33 (s, 1H), 7.72 (d, J=5.0 Hz, 1H), 7.56 (s, 1H), 7.10 (d, J=5.0 Hz, 1H), 6.12 (d, J=10.0 Hz, 2H), 5.73 (s, 1H), 4.44-4.39 (m, 1H), 4.05-4.02 (m, 1H), 3.92-3.89 (m, 2H), 3.66-3.64 (m, 2H), 3.24-3.23 (m, 1H), 2.90-2.59 (m, 6H), 2.45-2.39 (m, 1H), 2.20-2.15 (m, 2H), 2.11-2.02 (m, 3H), 1.95-1.90 (m, 3H), 1.80-1.71 (m, 2H), 1.63 (s, 6H), 1.43-1.35 (m, 3H), 1.02 (d, J=5.0 Hz, 3H). [M+H]⁺=819.7.

### Example 303: 3-cyano-N-(2-((1R,4r)-4-((R)-4-(1-(4-(2,4-dioxotetrahydropyrimidin-1(2H)-yl)-3,5-difluorophenyl)azetidine-3-carbonyl)-2-methylpiperazin-1-yl)cyclohexyl)-6-(2-hydroxypropan-2-yl)-2H-indazol-5-yl)pyrrolo[1,2-b]pyridazine-7-carboxamide

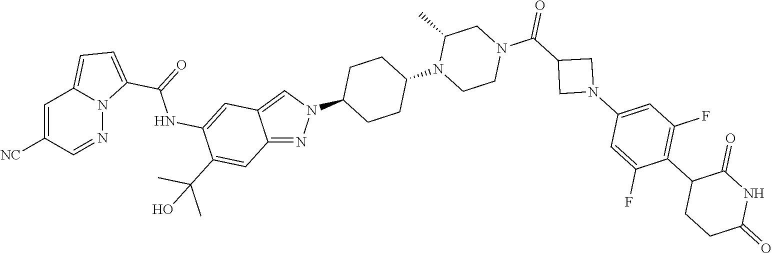 [SMILES: C[C@@H]1CN(C(=O)C2CN(c3cc(F)c(C4CCC(=O)NC4=O)c(F)c3)C2)CCN1[C@H]1CC[C@H](n2cc3cc(NC(=O)c4ccc5cc(C#N)cnn45)c(C(C)(C)O)cc3n2)CC1]

The title compound (35 mg, 41%) was prepared in a manner similar to that described in Example 185. ¹H NMR (500 MHz, DMSO) δ 12.03 (s, 1H), 10.50 (s, 1H), 8.94 (d, J=5.0 Hz, 1H), 8.74 (d, J=5.0 Hz, 1H), 8.55 (s, 1H), 8.33 (s, 1H), 7.73 (d, J=5.0 Hz, 1H), 7.57 (s, 1H), 7.10 (d, J=5.0 Hz, 1H), 6.26 (d, J=10.0 Hz, 2H), 5.74 (s, 1H), 4.44-4.40 (m, 1H), 4.09-3.82 (m, 6H), 3.61-3.58 (m, 2H), 3.17-3.11 (m, 1H), 2.96-2.84 (m, 3H), 2.74-2.2.64 (m, 3H), 2.36-2.27 (m, 1H), 2.19-2.15 (m, 2H), 2.07-1.87 (m, 3H), 1.80-1.71 (m, 2H), 1.63 (s, 6H), 1.41-1.35 (m, 1H), 1.02 (d, J=5.0 Hz, 3H). [M+H]+=848.7.

### Example 313: 3-cyano-N-(2-((1R,4r)-4-((R)-4-((R)-1-(4-((R)-2,6-dioxopiperidin-3-yl)-3,5-difluorophenyl)pyrrolidine-3-carbonyl)-3-(methoxymethyl)piperazin-1-yl)cyclohexyl)-6-(2-hydroxypropan-2-yl)-2H-indazol-5-yl)pyrrolo[1,2-b]pyridazine-7-carboxamide

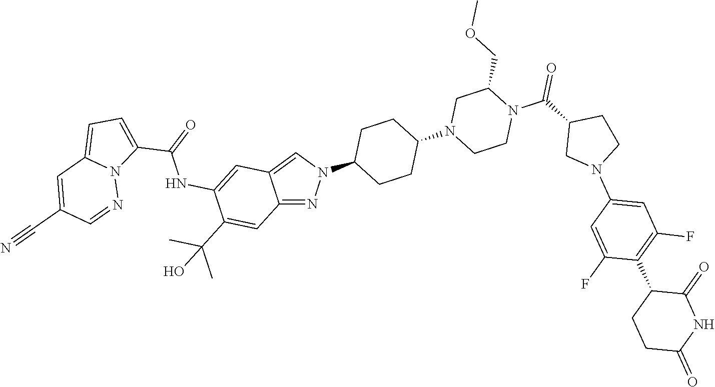 [SMILES: COC[C@H]1CN([C@H]2CC[C@H](n3cc4cc(NC(=O)c5ccc6cc(C#N)cnn56)c(C(C)(C)O)cc4n3)CC2)CCN1C(=O)[C@@H]1CCN(c2cc(F)c([C@H]3CCC(=O)NC3=O)c(F)c2)C1]

The title compound (31 mg, 40%) was prepared in a manner similar to that described in Example 185. ¹H NMR (500 MHz, DMSO) δ 12.02 (s, 1H), 10.84 (s, 1H), 8.94 (d, J=5.0 Hz, 1H), 8.74 (d, J=5.0 Hz, 1H), 8.55 (s, 1H), 8.33 (s, 1H), 7.72 (d, J=5.0 Hz, 1H), 7.58 (s, 1H), 7.10 (d, J=5.0 Hz, 1H), 6.23 (d, J=10.0 Hz, 2H), 5.73 (s, 1H), 4.58-3.84 (m, 1H), 4.43 (t, J=10.0 Hz, 1H), 4.27-4.22 (m, 1H), 4.04-4.00 (m, 1H), 3.72-3.51 (m, 3H), 3.45-3.42 (m, 1H), 3.30-3.20 (m, 8H), 2.98-2.74 (m, 3H), 2.45-2.39 (m, 1H), 2.29-2.05 (m, 7H), 1.95-1.90 (m, 5H), 1.63 (s, 6H), 1.52-1.45 (m, 2H). [M+H]+=891.5.

### Example 314: 3-cyano-N-(2-((1R,4r)-4-((R)-4-(4-((R)-2,6-dioxopiperidin-3-yl)-3,5-difluorophenethyl)-3-(methoxymethyl)piperazin-1-yl)cyclohexyl)-6-(2-hydroxypropan-2-yl)-2H-indazol-5-yl)pyrrolo[1,2-b]pyridazine-7-carboxamide

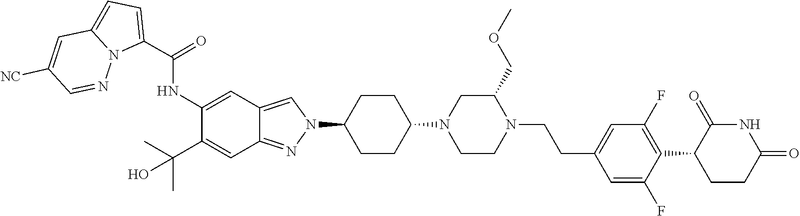 [SMILES: COC[C@H]1CN([C@H]2CC[C@H](n3cc4cc(NC(=O)c5ccc6cc(C#N)cnn56)c(C(C)(C)O)cc4n3)CC2)CCN1CCc1cc(F)c([C@H]2CCC(=O)NC2=O)c(F)c1]

To a solution of 3-cyano-N-(6-(2-hydroxypropan-2-yl)-2-((1R,4r)-4-((R)-3-(methoxymethyl)piperazin-1-yl)cyclohexyl)-2H-indazol-5-yl)pyrrolo[1,2-b]pyridazine-7-carboxamide (100 mg, 0.175 mmol) and (R)-2-(4-(2,6-dioxopiperidin-3-yl)-3,5-difluorophenyl)acetaldehyde (100 mg, 0.375 mmol) in DCE (30 mL) was added NaBH(Ac)₃ (111 mg, 0.525 mmol) at rt. The resulting mixture was stirred overnight at rt. The mixture was diluted with NaHCO₃ (saturated solvent) and extracted with DCM. The combined organic layer was dried over anhydrous Na₂SO₄, filtered and evaporated under vacuum. The residue was purified with Prep-HPLC (C-18 column chromatography (0.1% FA in water:acetonitrile=90:10-60:40 gradient elution) to afford the desired product (69 mg, 48%).

¹H NMR (500 MHz, DMSO) δ 12.02 (s, 1H), 10.95 (s, 1H), 8.94 (d, J=5.0 Hz, 1H), 8.74 (d, J=5.0 Hz, 1H), 8.55 (s, 1H), 8.33 (s, 1H), 7.72 (d, J=5.0 Hz, 1H), 7.57 (s, 1H), 7.10 (d, J=5.0 Hz, 1H), 7.01 (d, J=10.0 Hz, 2H), 5.72 (s, 1H), 4.48-4.43 (m, 1H), 4.22-4.18 (m, 1H), 3.51-3.52 (m, 1H), 3.33-3.25 (m, 5H), 2.85-2.60 (m, 10H), 2.43-2.36 (m, 2H), 2.17-2.07 (m, 4H), 2.01-1.93 (m, 5H), 1.63 (s, 6H), 1.52-1.45 (m, 2H). [M+H]⁺=822.6.

### Example 315: 3-cyano-N-(2-((1R,4r)-4-((R)-4-(1-(4-(2,4-dioxotetrahydropyrimidin-1(2H)-yl)-3,5-difluorophenyl)azetidine-3-carbonyl)-3-(methoxymethyl)piperazin-1-yl)cyclohexyl)-6-(2-hydroxypropan-2-yl)-2H-indazol-5-yl)pyrrolo[1,2-b]pyridazine-7-carboxamide

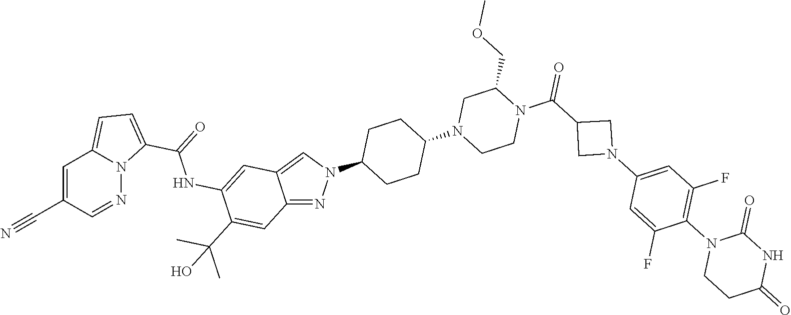 [SMILES: COC[C@H]1CN([C@H]2CC[C@H](n3cc4cc(NC(=O)c5ccc6cc(C#N)cnn56)c(C(C)(C)O)cc4n3)CC2)CCN1C(=O)C1CN(c2cc(F)c(N3CCC(=O)NC3=O)c(F)c2)C1]

The title compound (34 mg, 44%) was prepared in a manner similar to that described in Example 185. ¹H NMR (500 MHz, DMSO) δ 12.02 (s, 1H), 10.50 (s, 1H), 8.94 (d, J=5.0 Hz, 1H), 8.74 (d, J=5.0 Hz, 1H), 8.55 (s, 1H), 8.33 (s, 1H), 7.72 (d, J=5.0 Hz, 1H), 7.57 (s, 1H), 7.10 (d, J=5.0 Hz, 1H), 6.23 (d, J=10.0 Hz, 2H), 5.73 (s, 1H), 4.58-4.22 (m, 1H), 4.43 (t, J=10.0 Hz, 1H), 4.07-3.94 (m, 3H), 3.88-3.85 (m, 2H), 3.70-3.49 (m, 4H), 3.45-3.38 (m, 1H), 3.30 (s, 3H), 3.14-2.80 (m, 3H), 2.69-2.67 (m, 2H), 2.47-2.17 (in, 5H), 1.97-1.88 (m, 4H), 1.63 (s, 6H), 1.51-1.43 (m, 2H). [M+H]⁺=878.5.

### Example 316: 3-cyano-N-(2-((1R,4r)-4-((R)-4-((R)-1-(4-(2,4-dioxotetrahydropyrimidin-1(2H)-yl)-3,5-difluorophenyl)pyrrolidine-3-carbonyl)-3-(methoxymethyl)piperazin-1-yl)cyclohexyl)-6-(2-hydroxypropan-2-yl)-2H-indazol-5-yl)pyrrolo[1,2-b]pyridazine-7-carboxamide

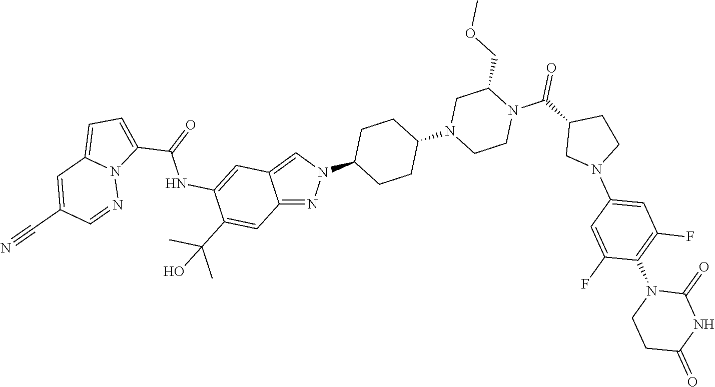

The title compound (26 mg, 33%) was prepared in a manner similar to that described in Example 185. ¹H NMR (500 MHz, DMSO) δ 12.03 (s, 1H), 10.48 (s, 1H), 8.93 (d, J=5.0 Hz, 1H), 8.73 (d, J=5.0 Hz, 1H), 8.55 (s, 1H), 8.33 (s, 1H), 7.72 (d, J=5.0 Hz, 1H), 7.57 (s, 1H), 7.10 (d, J=5.0 Hz, 1H), 6.32 (d, J=10.0 Hz, 2H), 5.73 (s, 1H), 4.58-4.23 (m, 1H), 4.43 (t, J=10.0 Hz, 1H), 3.87-3.51 (m, 6H), 3.47-3.19 (m, 7H), 2.98-2.80 (m, 2H), 2.70-2.67 (m, 2H), 2.48-2.39 (m, 1H), 2.29-2.07 (m, 5H), 1.95-1.90 (m, 4H), 1.63 (s, 6H), 1.52-1.44 (m, 2H). [M+H]⁺=892.5.

### Example 317: 3-cyano-N-(2-((1R,4r)-4-((R)-4-(1-(4-((R)-2,6-dioxopiperidin-3-yl)-3,5-difluorophenyl)piperidin-4-yl)-3-(methoxymethyl)piperazin-1-yl)cyclohexyl)-6-(2-hydroxypropan-2-yl)-2H-indazol-5-yl)pyrrolo[1,2-b]pyridazine-7-carboxamide

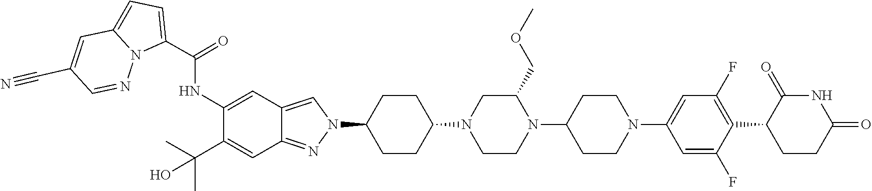 [SMILES: COC[C@H]1CN([C@H]2CC[C@H](n3cc4cc(NC(=O)c5ccc6cc(C#N)cnn56)c(C(C)(C)O)cc4n3)CC2)CCN1C1CCN(c2cc(F)c([C@H]3CCC(=O)NC3=O)c(F)c2)CC1]

The title compound (7 mg, 9%) was prepared in a manner similar to that described in Example 314. ¹H NMR (500 MHz, DMSO) δ 12.02 (s, 1H), 10.87 (s, 1H), 8.93 (d, J=5.0 Hz, 1H), 8.73 (d, J=5.0 Hz, 1H), 8.55 (s, 1H), 8.32 (s, 1H), 7.72 (d, J=5.0 Hz, 1H), 7.57 (s, 1H), 7.09 (d, J=5.0 Hz, 1H), 6.62 (d, J=10.0 Hz, 2H), 5.72 (s, 1H), 4.44-4.39 (m, 1H), 4.06-4.02 (m, 1H), 3.80-3.78 (m, 2H), 3.54-3.51 (m, 1H), 3.40-3.38 (m, 2H), 3.26 (s, 3H), 2.82-2.58 (m, 8H), 2.44-2.35 (m, 4H), 2.17-2.05 (m, 3H), 1.95-1.92 (m, 5H), 1.83-1.81 (m, 1H), 1.63-1.50 (m, 8H), 1.48-1.36 (m, 3H). [M+H]⁺=877.4.

### Example 318: 3-cyano-N-(2-((1R,4r)-4-((R)-4-(1-(4-((R)-2,6-dioxopiperidin-3-yl)-3,5-difluorophenyl)azetidin-3-yl)-3-(methoxymethyl)piperazin-1-yl)cyclohexyl)-6-(2-hydroxypropan-2-yl)-2H-indazol-5-yl)pyrrolo[1,2-b]pyridazine-7-carboxamide

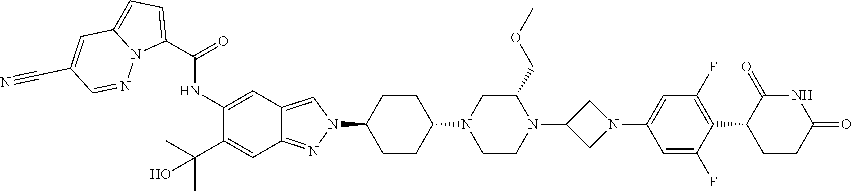 [SMILES: COC[C@H]1CN([C@H]2CC[C@H](n3cc4cc(NC(=O)c5ccc6cc(C#N)cnn56)c(C(C)(C)O)cc4n3)CC2)CCN1C1CN(c2cc(F)c([C@H]3CCC(=O)NC3=O)c(F)c2)C1]

The title compound (11 mg, 15%) was prepared in a manner similar to that described in Example 314. ¹H NMR (500 MHz, DMSO) δ 12.02 (s, 1H), 10.86 (s, 1H), 8.93 (d, J=5.0 Hz, 1H), 8.73 (d, J=5.0 Hz, 1H), 8.55 (s, 1H), 8.32 (s, 1H), 7.72 (d, J=5.0 Hz, 1H), 7.57 (s, 1H), 7.10 (d, J=5.0 Hz, 1H), 6.13 (d, J=10.0 Hz, 2H), 5.73 (s, 1H), 4.44-4.39 (m, 1H), 4.05-4.01 (m, 1H), 3.95-3.88 (m, 2H), 3.71-3.64 (m, 2H), 3.60-3.58 (m, 1H), 3.51-3.48 (m, 2H), 3.26 (s, 3H), 2.80-2.74 (m, 1H), 2.68-2.67 (m, 2H), 2.59-2.48 (m, 5H), 2.41-2.33 (m, 2H), 2.18-2.06 (m, 3H), 1.95-1.92 (m, 5H), 1.63 (s, 6H), 1.48-1.43 (m, 2H). [M+H]⁺=849.3.

### Example 325: 3-cyano-N-(2-((1S,4r)-4-((S)-4-(4-((R)-2,6-dioxopiperidin-3-yl)-3,5-difluorophenethyl)-2-(methoxymethyl)piperazin-1-yl)cyclohexyl)-6-(2-hydroxypropan-2-yl)-2H-indazol-5-yl)pyrrolo[1,2-b]pyridazine-7-carboxamide

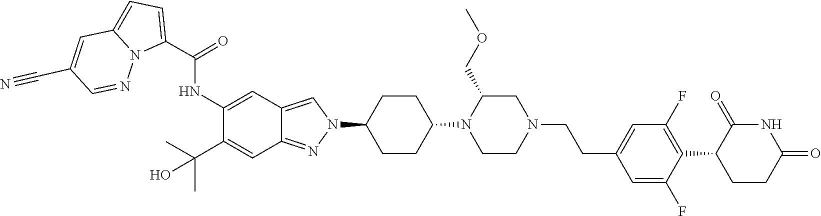 [SMILES: COC[C@@H]1CN(CCc2cc(F)c([C@H]3CCC(=O)NC3=O)c(F)c2)CCN1[C@H]1CC[C@H](n2cc3cc(NC(=O)c4ccc5cc(C#N)cnn45)c(C(C)(C)O)cc3n2)CC1]

To a solution of 3-cyano-N-(6-(2-hydroxypropan-2-yl)-2-((1S,4r)-4-((S)-2-(methoxymethyl)piperazin-1-yl)cyclohexyl)-2H-indazol-5-yl)pyrrolo[1,2-b]pyridazine-7-carboxamide (100 mg, 0.175 mmol) and (R)-2-(4-(2,6-dioxopiperidin-3-yl)-3,5-difluorophenyl)acetaldehyde (100 mg, 0.375 mmol) in DCE (30 mL) was added NaBH(Ac)₃ (111 mg, 0.525 mmol) at rt. The resulting mixture was stirred overnight at rt. The mixture was diluted with NaHCO₃ (saturated solvent) and extracted with DCM. The combined organic layer was dried over anhydrous Na₂SO₄, filtered and evaporated under vacuum. The residue was purified with Prep-HPLC (C-18 column chromatography (0.1% FA in water:acetonitrile=90:10˜60:40 gradient elution) to afford the desired product (46 mg, 31.9%).

¹H NMR (500 MHz, DMSO) δ 12.02 (s, 1H), 10.94 (s, 1H), 8.94 (d, J=5.0 Hz, 1H), 8.74 (d, J=5.0 Hz, 1H), 8.55 (s, 1H), 8.34 (s, 1H), 7.72 (d, J=5.0 Hz, 1H), 7.57 (s, 1H), 7.10 (d, J=5.0 Hz, 1H), 7.03 (d, J=10.0 Hz, 2H), 5.72 (s, 1H), 4.44-4.39 (m, 1H), 4.21-4.18 (m, 1H), 3.52-3.49 (m, 1H), 3.36-3.35 (m, 2H), 3.27 (s, 3H), 2.83-2.71 (m, 6H), 2.63-2.54 (m, 4H), 2.48-2.45 (m, 1H), 2.25-2.09 (m, 5H), 2.01-1.90 (m, 4H), 1.80-1.78 (m, 1H), 1.68-1.60 (m, 7H), 1.41-1.39 (m, 1H). [M+H]⁺=822.7.

### Example 326: 3-cyano-N-(2-((1S,4r)-4-((S)-4-((1-(4-((R)-2,6-dioxopiperidin-3-yl)-3,5-difluorophenyl)azetidin-3-yl)methyl)-2-(methoxymethyl)piperazin-1-yl)cyclohexyl)-6-(2-hydroxypropan-2-yl)-2H-indazol-5-yl)pyrrolo[1,2-b]pyridazine-7-carboxamide

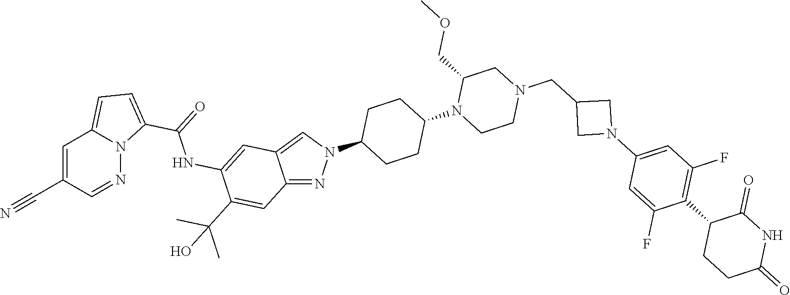 [SMILES: COC[C@@H]1CN(CC2CN(c3cc(F)c([C@H]4CCC(=O)NC4=O)c(F)c3)C2)CCN1[C@H]1CC[C@H](n2cc3cc(NC(=O)c4ccc5cc(C#N)cnn45)c(C(C)(C)O)cc3n2)CC1]

The title compound (74 mg, 49%) was prepared in a manner similar to that described in Example 325. ¹H NMR (500 MHz, DMSO) δ 12.02 (s, 1H), 10.85 (s, 1H), 8.94 (d, J=5.0 Hz, 1H), 8.74 (d, J=5.0 Hz, 1H), 8.55 (s, 1H), 8.34 (s, 1H), 7.72 (d, J=5.0 Hz, 1H), 7.57 (s, 1H), 7.10 (d, J=5.0 Hz, 1H), 6.12 (d, J=10.0 Hz, 2H), 5.72 (s, 1H), 4.44-4.39 (m, 1H), 4.04-4.01 (m, 1H), 3.94-3.91 (m, 2H), 3.51-3.46 (m, 3H), 3.35-3.32 (m, 2H), 3.28 (s, 3H), 2.83-2.71 (m, 5H), 2.62-2.54 (m, 4H), 2.48-2.43 (m, 1H), 2.17-2.16 (m, 3H), 2.08-1.87 (m, 6H), 1.78-1.76 (m, 1H), 1.70-1.63 (m, 7H), 1.43-1.36 (m, 1H). [M+H]⁺=863.6.

This compound could also be synthesized in another manner.

### Step 1: methyl 2-((1S,4r)-4-((S)-4-(tert-butoxycarbonyl)-2-(methoxymethyl)piperazin-1-yl)cyclohexyl)-2H-indazole-6-carboxylate

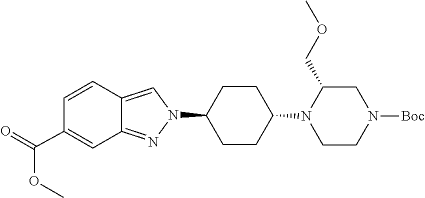 [SMILES: COC[C@@H]1CN(C(=O)OC(C)(C)C)CCN1[C@H]1CC[C@H](n2cc3ccc(C(=O)OC)cc3n2)CC1]

To a solution of methyl 4-formyl-3-nitrobenzoate (6.4 g, 30.5 mmol) in propan-2-ol (100 mL) was added tert-butyl (S)-4-((1r,4S)-4-aminocyclohexyl)-3-(methoxymethyl)piperazine-1-carboxylate (10.0 g, 30.5 mmol). The mixture was stirred for 3 hours at 80° C. under N₂. To the above solution was added tri-n-butylphosphane (18.5 g, 91.5 mmol). The mixture was stirred 12 hours at 80° C. under N₂. The mixture was cooled to rt and concentrated under vacuum. The residue was slurried with petroleum ether (200 mL) and filtered. The solid was filtered and dried under vacuum to yield product (10.5 g, 70.5%). [M+H]⁺=487.3.

### Step 2: methyl 2-((1S,4r)-4-((S)-2-(methoxymethyl)piperazin-1-yl)cyclohexyl)-5-nitro-2H-indazole-6-carboxylate

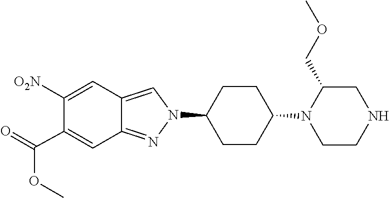 [SMILES: COC[C@@H]1CNCCN1[C@H]1CC[C@H](n2cc3cc([N+](=O)[O-])c(C(=O)OC)cc3n2)CC1]

To a solution of methyl 2-((1S,4r)-4-((S)-4-(tert-butoxycarbonyl)-2-(methoxymethyl)piperazin-1-yl)cyclohexyl)-2H-indazole-6-carboxylate (10.5 g, 21.5 mmol) in conc.H₂SO₄ (40 mL) was slowed added NaNO₃ (2.1 g, 32.3 mmol) batchwise at 0° C. The mixture was stirred for 2 hours at 0° C. The mixture was quenched with ice-water, then adjusted to pH 9 with sat.Na₂CO₃ solution and extracted with DCM. The combined organic layer was dried over anhydrous Na₂SO₄, filtered and evaporated in vacuum to afford product (9 g, 97.0%), which was used without further purification. [M+H]⁺=432.2.

### Step 3: methyl 2-((1S,4r)-4-((S)-4-(tert-butoxycarbonyl)-2-(methoxymethyl)piperazin-1-yl)cyclohexyl)-5-nitro-2H-indazole-6-carboxylate

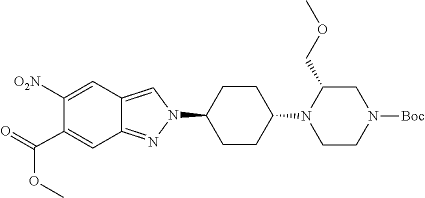 [SMILES: COC[C@@H]1CN(C(=O)OC(C)(C)C)CCN1[C@H]1CC[C@H](n2cc3cc([N+](=O)[O-])c(C(=O)OC)cc3n2)CC1]

To a solution of methyl 2-((1S,4r)-4-((S)-2-(methoxymethyl)piperazin-1-yl)cyclohexyl)-5-nitro-2H-indazole-6-carboxylate (9 g, 20.8 mmol) in DCM (100 mL) was added DIEA (5.4 g, 41.6 mmol) and (Boc)₂O (5.4 g, 24.96 mmol). The reaction mixture was stirred overnight at rt. The mixture was extracted with DCM (3×300 mL), dried over Na₂SO₄ and concentrated under vacuum. The residue was purified with silica gel column chromatography (DCM:MeOH=30:1) to yield product (7.7 g, 70%). [M+H]⁺=532.2

### Step 4: methyl 5-amino-2-((1S,4r)-4-((S)-4-(tert-butoxycarbonyl)-2-(methoxymethyl)piperazin-1-yl)cyclohexyl)-2H-indazole-6-carboxylate

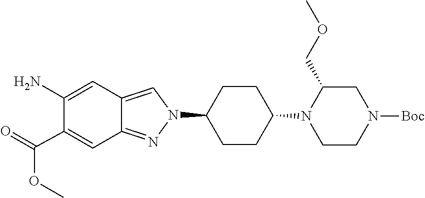 [SMILES: COC[C@@H]1CN(C(=O)OC(C)(C)C)CCN1[C@H]1CC[C@H](n2cc3cc(N)c(C(=O)OC)cc3n2)CC1]

To a solution of methyl 2-((1S,4r)-4-((S)-4-(tert-butoxycarbonyl)-2-(methoxymethyl)piperazin-1-yl)cyclohexyl)-5-nitro-2H-indazole-6-carboxylate (7.7 g, 14.5 mmol) in MeOH (100 mL) and water (20 mL) was added NH₄Cl (3.9 g, 72.5 mmol) and Fe powder (4.1 g, 72.5 mmol) at room temperature. The resulting mixture was stirred for 2 h at 70° C. The mixture was cooled to rt and concentrated under vacuum. The residue was purified by silica gel column chromatography (DCM:MeOH=15:1) to afford product (6.0 g, 82%). [M+H]⁺=502.3.

### Step 5: methyl 2-((1S,4r)-4-((S)-4-(tert-butoxycarbonyl)-2-(methoxymethyl)piperazin-1-yl)cyclohexyl)-5-(2,2,2-trifluoroacetamido)-2H-indazole-6-carboxylate

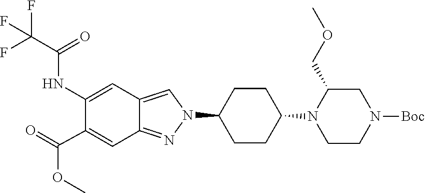 [SMILES: COC[C@@H]1CN(C(=O)OC(C)(C)C)CCN1[C@H]1CC[C@H](n2cc3cc(NC(=O)C(F)(F)F)c(C(=O)OC)cc3n2)CC1]

To a solution of methyl 5-amino-2-((1S,4r)-4-((S)-4-(tert-butoxycarbonyl)-2-(methoxymethyl)piperazin-1-yl)cyclohexyl)-2H-indazole-6-carboxylate (6.0 g, 11.95 mmol) in DCM (100 mL) was added (CF₃CO)₂O (3.0 g, 14.34 mmol). The reaction mixture was stirred overnight at rt. The mixture was concentrated under vacuum. The residue was purified with silica gel column chromatography (DCM:MeOH=30:1) to yield product (6.5 g, 91%). [M+H]⁺=598.3

### Step 6: tert-butyl (S)-4-((1r,4S)-4-(6-(2-hydroxypropan-2-yl)-5-(2,2,2-trifluoroacetamido)-2H-indazol-2-yl)cyclohexyl)-3-(methoxymethyl)piperazine-1-carboxylate

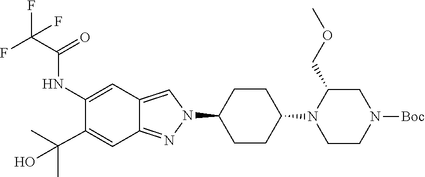 [SMILES: COC[C@@H]1CN(C(=O)OC(C)(C)C)CCN1[C@H]1CC[C@H](n2cc3cc(NC(=O)C(F)(F)F)c(C(C)(C)O)cc3n2)CC1]

To a solution of methyl 2-((1S,4r)-4-((S)-4-(tert-butoxycarbonyl)-2-(methoxymethyl)piperazin-1-yl)cyclohexyl)-5-(2,2,2-trifluoroacetamido)-2H-indazole-6-carboxylate (6.5 g, 10.87 mmol) and LiCl (1.8 g, 43.48 mmol) in THF (100 mL) was added dropwise MeMgBr in THF (3.2 M, 27 mL, 86.96 mmol) at 0° C. The resulting mixture was stirred for 2 h at 0° C. The mixture was quenched with sat.NH₄Cl solution and extracted with DCM. The combined organic layer was dried over anhydrous Na₂SO₄, filtered and evaporated under vacuum. The residue was purified on silica gel column chromatography (DCM:MeOH=10:1) to afford product (6 g, 92%). [M+H]⁺=598.3

### Step 7: tert-butyl (S)-4-((1r,4S)-4-(5-amino-6-(2-hydroxypropan-2-yl)-2H-indazol-2-yl)cyclohexyl)-3-(methoxymethyl)piperazine-1-carboxylate

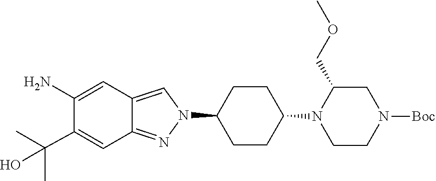 [SMILES: COC[C@@H]1CN(C(=O)OC(C)(C)C)CCN1[C@H]1CC[C@H](n2cc3cc(N)c(C(C)(C)O)cc3n2)CC1]

To a solution of tert-butyl (S)-4-((1r,4S)-4-(6-(2-hydroxypropan-2-yl)-5-(2,2,2-trifluoroacetamido)-2H-indazol-2-yl)cyclohexyl)-3-(methoxymethyl)piperazine-1-carboxylate (6.0 g, 10.0 mmol) in MeOH (50 mL) was added dropwise NaOH in H₂O (2.0 M, 50 mL, 100 mmol) at 0° C. The resulting mixture was stirred for 5 h at 70° C. The mixture was extracted with DCM. The combined organic layer was dried over anhydrous Na₂SO₄, filtered and evaporated under vacuum. The residue was purified on silica gel column chromatography (DCM:MeOH=10:1) to afford product (5.0 g, 99.5%). [M+H]⁺=502.3

### Step 8: tert-butyl (S)-4-((1r,4S)-4-(5-(3-cyanopyrrolo[1,2-b]pyridazine-7-carboxamido)-6-(2-hydroxypropan-2-yl)-2H-indazol-2-yl)cyclohexyl)-3-(methoxymethyl)piperazine-1-carboxylate

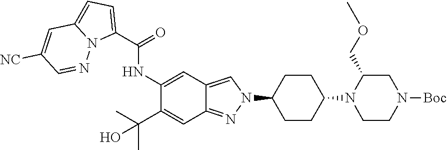 [SMILES: COC[C@@H]1CN(C(=O)OC(C)(C)C)CCN1[C@H]1CC[C@H](n2cc3cc(NC(=O)c4ccc5cc(C#N)cnn45)c(C(C)(C)O)cc3n2)CC1]

To a stirred solution of 3-cyanopyrrolo[1,2-b]pyridazine-7-carboxylic acid (744 mg, 3.98 mmol) and tert-butyl (S)-4-((1r,4S)-4-(5-amino-6-(2-hydroxypropan-2-yl)-2H-indazol-2-yl)cyclohexyl)-3-(methoxymethyl)piperazine-1-carboxylate (2.5 g, 4.98 mmol) in DMF (210 mL) was added HATU (2.84 g, 7.47 mmol) and DIEA (1.3 g, 9.96 mmol). The solution was stirred at rt for 4 h. Then the solution was diluted with EA (200 mL), washed with water and brine. The organic layer was dried over Na₂SO₄ and concentrated under vacuum. The residue was purified with Combi-Flash (silica column, 20 g, DCM:MeOH=20:1) to yield the product (3.0 g, 89.8%). [M+H]⁺=671.4.

### Step 9: 3-cyano-N-(6-(2-hydroxypropan-2-yl)-2-((1S,4r)-4-((S)-2-(methoxymethyl)piperazin-1-yl)cyclohexyl)-2H-indazol-5-yl)pyrrolo[1,2-b]pyridazine-7-carboxamide

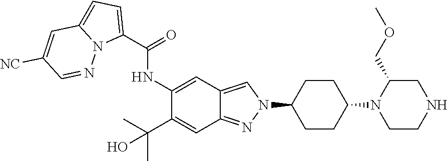 [SMILES: COC[C@@H]1CNCCN1[C@H]1CC[C@H](n2cc3cc(NC(=O)c4ccc5cc(C#N)cnn45)c(C(C)(C)O)cc3n2)CC1]

To a stirring solution of tert-butyl (S)-4-((1r,4S)-4-(5-(3-cyanopyrrolo[1,2-b]pyridazine-7-carboxamido)-6-(2-hydroxypropan-2-yl)-2H-indazol-2-yl)cyclohexyl)-3-(methoxymethyl)piperazine-1-carboxylate (3.0 g, 4.47 mmol) in DCM (40 mL) and MeOH (40 mL) was slowly droppwised con. HCl (40 mL) at 0° C. The reaction mixture was stirred at rt for 2 h at r.t. and to the mixture was added H₂O (100 ml), then adjusted the pH>8 with NaHCO₃ solid. The mixture was extracted with DCM (200 ml×6), the organic layer was dried with Na2SO4, filtered and concentrated to afford the crude product (2.3 g, 90.2%). [M+H]+=571.3.

### Step 10: 3-cyano-N-(2-((1S,4r)-4-((S)-4-((1-(4-((R)-2,6-dioxopiperidin-3-yl)-3,5-difluorophenyl)azetidin-3-yl)methyl)-2-(methoxymethyl)piperazin-1-yl)cyclohexyl)-6-(2-hydroxypropan-2-yl)-2H-indazol-5-yl)pyrrolo[1,2-b]pyridazine-7-carboxamide

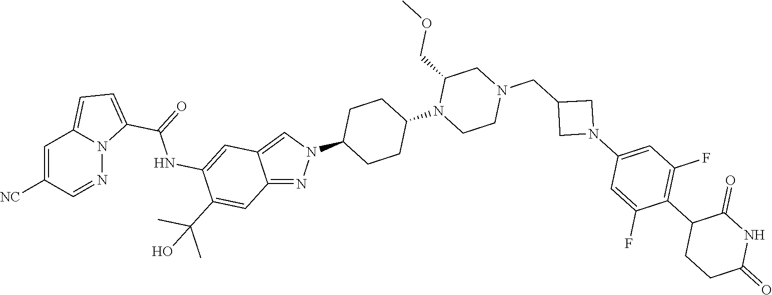 [SMILES: COC[C@@H]1CN(CC2CN(c3cc(F)c(C4CCC(=O)NC4=O)c(F)c3)C2)CCN1[C@H]1CC[C@H](n2cc3cc(NC(=O)c4ccc5cc(C#N)cnn45)c(C(C)(C)O)cc3n2)CC1]

A solution of 3-cyano-N-(6-(2-hydroxypropan-2-yl)-2-((1S,4r)-4-((S)-2-(methoxymethyl)piperazin-1-yl)cyclohexyl)-2H-indazol-5-yl)pyrrolo[1,2-b]pyridazine-7-carboxamide (1.5 g, 2.63 mmol) and (R)-1-(4-(2,6-dioxopiperidin-3-yl)-3,5-difluorophenyl)azetidine-3-carbaldehyde (CAS: 2873368-99-9, which was obtained in the same way described in WO2022012622A1) (972 mg, 3.156 mmol) in DCE (50 mL) was stirred at room temperature for 0.5 hours, then NaBH(OAc)₃ (836 mg, 3.945 mmol) was added portion wise. The solution was stirred at room temperature for another 2 hours. Then the mixture was purified with Combi-Flash (silica column, 80 g, DCM:MeOH=10:1) to yield the product (1.6 g, 70.5%). [M+H]⁺=863.6.

### Example 327: 3-cyano-N-(2-((1S,4r)-4-((S)-4-(1-(4-((R)-2,6-dioxopiperidin-3-yl)-3,5-difluorophenyl)azetidin-3-yl)-2-(methoxymethyl)piperazin-1-yl)cyclohexyl)-6-(2-hydroxypropan-2-yl)-2H-indazol-5-yl)pyrrolo[1,2-b]pyridazine-7-carboxamide

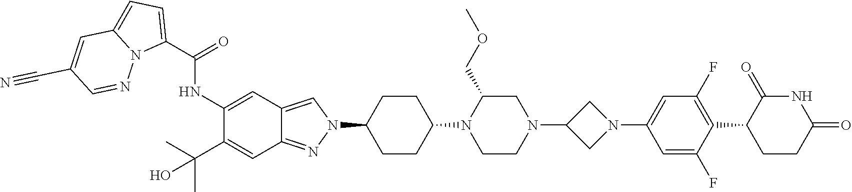 [SMILES: COC[C@@H]1CN(C2CN(c3cc(F)c([C@H]4CCC(=O)NC4=O)c(F)c3)C2)CCN1[C@H]1CC[C@H](n2cc3cc(NC(=O)c4ccc5cc(C#N)cnn45)c(C(C)(C)O)cc3n2)CC1]

The title compound (47 mg, 32%) was prepared in a manner similar to that described in Example 325. ¹H NMR (500 MHz, DMSO) δ 12.02 (s, 1H), 10.86 (s, 1H), 8.94 (d, J=5.0 Hz, 1H), 8.74 (d, J=5.0 Hz, 1H), 8.55 (s, 1H), 8.34 (s, 1H), 7.72 (d, J=5.0 Hz, 1H), 7.57 (s, 1H), 7.10 (d, J=5.0 Hz, 1H), 6.13 (d, J=10.0 Hz, 2H), 5.72 (s, 1H), 4.42-4.39 (m, 1H), 4.05-4.01 (m, 1H), 3.92-3.90 (m, 2H), 3.64-3.61 (m, 2H), 3.54-3.52 (m, 1H), 3.36-3.21 (m, 6H), 2.84-2.74 (m, 4H), 2.57-2.51 (m, 3H), 2.15-1.90 (m, 9H), 1.79-1.75 (m, 1H), 1.68-1.63 (m, 7H), 1.43-1.40 (m, 1H). [M+H]⁺=849.6.

### Example 328: 3-cyano-N-(2-((1S,4r)-4-((S)-4-(1-(4-((R)-2,6-dioxopiperidin-3-yl)-3,5-difluorophenyl)piperidin-4-yl)-2-(methoxymethyl)piperazin-1-yl)cyclohexyl)-6-(2-hydroxypropan-2-yl)-2H-indazol-5-yl)pyrrolo[1,2-b]pyridazine-7-carboxamide

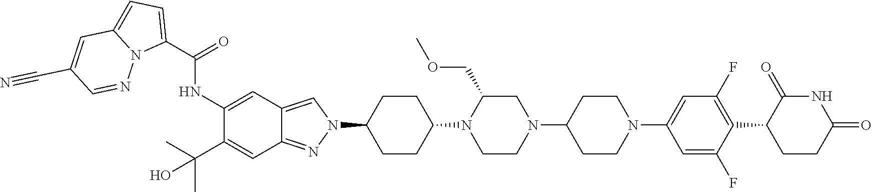 [SMILES: COC[C@@H]1CN(C2CCN(c3cc(F)c([C@H]4CCC(=O)NC4=O)c(F)c3)CC2)CCN1[C@H]1CC[C@H](n2cc3cc(NC(=O)c4ccc5cc(C#N)cnn45)c(C(C)(C)O)cc3n2)CC1]

The title compound (4 mg, 3%) was prepared in a manner similar to that described in Example 325.

¹H NMR (500 MHz, DMSO) δ 12.02 (s, 1H), 10.87 (s, 1H), 8.94 (d, J=5.0 Hz, 1H), 8.74 (d, J=5.0 Hz, 1H), 8.55 (s, 1H), 8.34 (s, 1H), 7.72 (d, J=5.0 Hz, 1H), 7.57 (s, 1H), 7.10 (d, J=5.0 Hz, 1H), 6.63 (d, J=10.0 Hz, 2H), 5.73 (s, 1H), 4.43-4.39 (m, 1H), 4.05-4.03 (m, 1H), 3.82-3.76 (m, 2H), 3.52-3.50 (m, 2H), 3.27 (s, 3H), 2.79-2.64 (m, 8H), 2.52-2.51 (m, 1H), 2.44-2.42 (m, 1H), 2.34-2.28 (m, 2H), 2.19-2.15 (m, 3H), 2.08-1.89 (m, 5H), 1.82-1.76 (m, 3H), 1.69-1.63 (m, 7H), 1.42-1.39 (m, 3H). [M+H]⁺=877.8.

### Example 334: 3-cyano-N-(2-((1R,4r)-4-(4-(4-((R)-2,6-dioxopiperidin-3-yl)-3,5-difluorophenethyl)-2-oxopiperazin-1-yl)cyclohexyl)-6-(2-hydroxypropan-2-yl)-2H-indazol-5-yl)pyrrolo[1,2-b]pyridazine-7-carboxamide

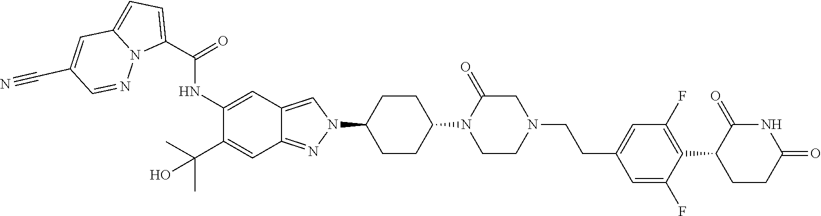 [SMILES: CC(C)(O)c1cc2nn([C@H]3CC[C@H](N4CCN(CCc5cc(F)c([C@H]6CCC(=O)NC6=O)c(F)c5)CC4=O)CC3)cc2cc1NC(=O)c1ccc2cc(C#N)cnn12]

The title compound (11 mg, 14%) was prepared in a manner similar to that described in Example 314. ¹H NMR (500 MHz, DMSO) δ 12.03 (s, 1H), 10.95 (s, 1H), 8.94 (d, J=5.0 Hz, 1H), 8.74 (d, J=5.0 Hz, 1H), 8.55 (s, 1H), 8.38 (s, 1H), 7.73 (d, J=5.0 Hz, 1H), 7.58 (s, 1H), 7.10 (d, J=5.0 Hz, 1H), 7.05 (d, J=10.0 Hz, 2H), 5.74 (s, 1H), 4.50-4.36 (m, 2H), 4.22-4.18 (m, 1H), 3.27-3.25 (m, 2H), 3.10 (s, 2H), 2.81-2.78 (m, 3H), 2.72-2.70 (m, 2H), 2.64-2.61 (m, 2H), 2.55-2.53 (m, 1H), 2.21-1.99 (m, 6H), 1.85-1.81 (m, 2H), 1.72-1.70 (m, 2H), 1.64 (s, 6H). [M+H]⁺=792.6.

### Example 335: 3-cyano-N-(2-((1R,4r)-4-(4-(1-(4-((R)-2,6-dioxopiperidin-3-yl)-3,5-difluorophenyl)azetidin-3-yl)-2-oxopiperazin-1-yl)cyclohexyl)-6-(2-hydroxypropan-2-yl)-2H-indazol-5-yl)pyrrolo[1,2-b]pyridazine-7-carboxamide

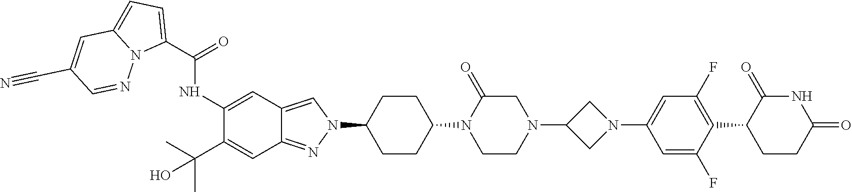 [SMILES: CC(C)(O)c1cc2nn([C@H]3CC[C@H](N4CCN(C5CN(c6cc(F)c([C@H]7CCC(=O)NC7=O)c(F)c6)C5)CC4=O)CC3)cc2cc1NC(=O)c1ccc2cc(C#N)cnn12]

The title compound (19 mg, 23%) was prepared in a manner similar to that described in Example 314. ¹H NMR (500 MHz, DMSO) δ 12.03 (s, 1H), 10.86 (s, 1H), 8.94 (d, J=5.0 Hz, 1H), 8.74 (d, J=5.0 Hz, 1H), 8.56 (s, 1H), 8.37 (s, 1H), 7.72 (d, J=5.0 Hz, 1H), 7.58 (s, 1H), 7.10 (d, J=5.0 Hz, 1H), 6.12 (d, J=10.0 Hz, 2H), 5.73 (s, 1H), 4.50-4.37 (m, 2H), 4.05-4.02 (m, 1H), 3.95-3.92 (m, 2H), 3.73-3.70 (m, 2H), 3.41-3.36 (m, 1H), 3.30-3.28 (m, 3H), 3.06 (s, 2H), 2.82-2.74 (m, 1H), 2.66-2.64 (m, 2H), 2.21-2.19 (m, 2H), 2.09-2.01 (m, 3H), 1.95-1.93 (m, 1H), 1.87-1.80 (m, 2H), 1.73-1.73 (m, 2H), 1.63 (s, 6H). [M+H]⁺=819.6.

### Example 210: 3-cyano-N-(2-((1r,4r)-4-(4-(1-(4-(2,4-dioxotetrahydropyrimidin-1(2H)-yl)-3-fluoro-5-methoxyphenyl)azetidine-3-carbonyl)piperazin-1-yl)cyclohexyl)-6-(2-hydroxypropan-2-yl)-2H-indazol-5-yl)pyrrolo[1,2-b]pyridazine-7-carboxamide

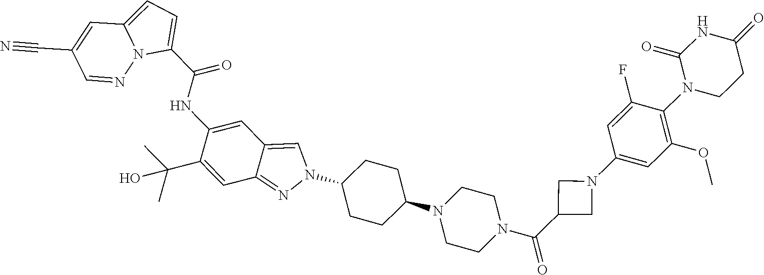 [SMILES: COc1cc(N2CC(C(=O)N3CCN([C@H]4CC[C@H](n5cc6cc(NC(=O)c7ccc8cc(C#N)cnn78)c(C(C)(C)O)cc6n5)CC4)CC3)C2)cc(F)c1N1CCC(=O)NC1=O]

The title compound (20 mg, 31%) was prepared in a manner similar to that described in Example 178. ¹H NMR (500 MHz, DMSO) δ 12.02 (s, 1H), 10.31 (s, 1H), 8.94 (s, 1H), 8.74 (s, 1H), 8.55 (s, 1H), 8.34 (s, 1H), 7.72 (s, 1H), 7.57 (s, 1H), 7.09 (s, 1H), 5.93 (s, 2H), 5.72 (s, 1H), 4.49-4.38 (m, 1H), 4.11-4.01 (m, 2H), 3.97-3.91 (m, 2H), 3.90-3.67 (m, 5H), 3.59-3.39 (m, 5H), 2.66-2.61 (m, 2H), 2.58-2.53 (m, 3H), 2.39-2.29 (m, 2H), 2.21-2.14 (m, 2H), 2.01-1.89 (m, 4H), 1.63 (s, 6H), 1.53-1.45 (m, 2H). [M+H]⁺=846.2.

### Example 263: 3-cyano-N-(2-((1R,4r)-4-(4-((R)-1-(4-(2,4-dioxotetrahydropyrimidin-1(2H)-yl)phenyl)pyrrolidine-3-carbonyl)piperazin-1-yl)cyclohexyl)-6-(2-hydroxypropan-2-yl)-2H-indazol-5-yl)pyrrolo[1,2-b]pyridazine-7-carboxamide

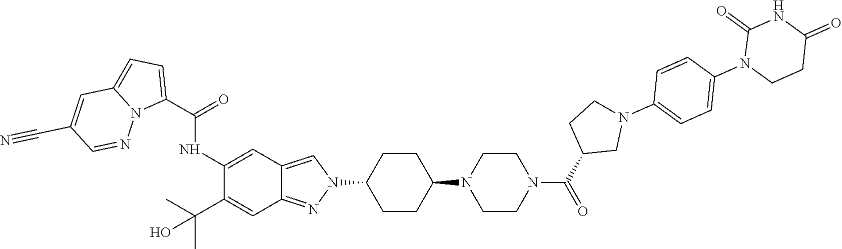 [SMILES: CC(C)(O)c1cc2nn([C@H]3CC[C@H](N4CCN(C(=O)[C@@H]5CCN(c6ccc(N7CCC(=O)NC7=O)cc6)C5)CC4)CC3)cc2cc1NC(=O)c1ccc2cc(C#N)cnn12]

The title compound (30 mg, 37%) was prepared in a manner similar to that described in Example 178. ¹H NMR (500 MHz, DMSO) δ 12.02 (s, 1H), 10.21 (s, 1H), 8.93 (d, J=1.5 Hz, 1H), 8.74 (d, J=2.0 Hz, 1H), 8.55 (s, 1H), 8.34 (s, 1H), 8.20 (d, J=4.0 Hz, 1H), 7.72 (d, J=4.5 Hz, 1H), 7.57 (s, 1H), 7.13-7.06 (m, 3H), 6.54 (d, J=8.5 Hz, 2H), 4.50-4.37 (m, 1H), 3.67 (t, J=6.5 Hz, 2H), 3.59-3.54 (m, 2H), 3.54-3.42 (m, 4H), 3.36-3.33 (m, 3H), 3.30-3.24 (m, 3H), 2.68 (t, J=6.5 Hz, 2H), 2.61-2.55 (m, 2H), 2.22-2.14 (m, 3H), 2.14-2.06 (m, 1H), 2.01-1.89 (m, 4H), 1.63 (s, 6H), 1.56-1.43 (m, 2H). [M+H]+=812.7.

### Example 287: 3-cyano-N-(2-((S)-1′-(1-(4-((R)-2,6-dioxopiperidin-3-yl)-3,5-difluorophenyl)azetidine-3-carbonyl)-3′,3′-difluoro-[1,4′-bipiperidin]-4-yl)-6-(2-hydroxypropan-2-yl)-2H-indazol-5-yl)pyrrolo[1,2-b]pyridazine-7-carboxamide

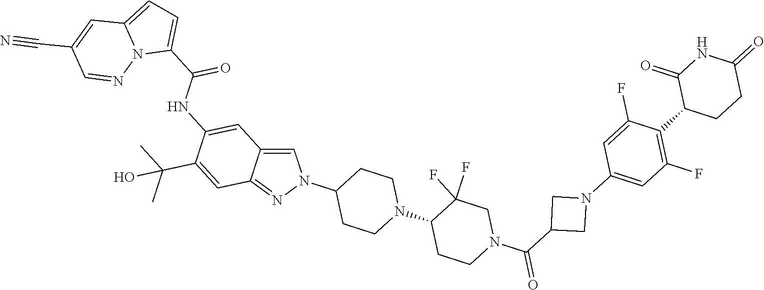 [SMILES: CC(C)(O)c1cc2nn(C3CCN([C@H]4CCN(C(=O)C5CN(c6cc(F)c([C@H]7CCC(=O)NC7=O)c(F)c6)C5)CC4(F)F)CC3)cc2cc1NC(=O)c1ccc2cc(C#N)cnn12]

To a solution of (S)-3-cyano-N-(2-(3′,3′-difluoro-[1,4′-bipiperidin]-4-yl)-6-(2-hydroxypropan-2-yl)-2H-indazol-5-yl)pyrrolo[1,2-b]pyridazine-7-carboxamide (56 mg, 0.1 mmol) and (R)-1-(4-(2,6-dioxopiperidin-3-yl)-3,5-difluorophenyl)azetidine-3-carboxylic acid (32 mg, 0.1 mmol) in DCM (5 mL) was added DIPEA (129 mg, 1 mmol) and T₃P in EA (50%, 128 mg, 0.2 mmol) at room temperature. The resulting mixture was stirred for 2 hours at room temperature. The mixture was added water (5 mL) and extracted with DCM (5 mL×3). The combined organic layer was dried over anhydrous Na₂SO₄, filtered and evaporated under vacuum. The residue was purified with Prep-HPLC (C-18 column chromatography (0.1% FA in water:acetonitrile=90:10˜60:40 gradient elution) to afford the desired product (55 mg, 63%). ¹H NMR (500 MHz, DMSO) δ 12.03 (s, 1H), 10.86 (s, 1H), 8.94 (d, J=1.5 Hz, 1H), 8.74 (s, 1H), 8.56 (s, 1H), 8.38 (s, 1H), 7.72 (d, J=4.5 Hz, 1H), 7.58 (s, 1H), 7.09 (d, J=4.5 Hz, 1H), 6.18 (t, J=11.0 Hz, 2H), 4.64-4.41 (m, 2H), 4.12-4.01 (m, 3H), 3.91 (s, 3H), 3.84-3.70 (m, 1H), 3.50-3.38 (m, 2H), 3.29-2.95 (m, 6H), 2.83-2.71 (m, 3H), 2.13-2.04 (m, 4H), 1.97-1.90 (m, 1H), 1.89-1.82 (m, 1H), 1.63 (s, 6H), 1.57-1.43 (m, 1H). [M-17]+=851.6.

### Example 339: 3-cyano-N-(2-((S)-1′-(4-((R)-2,6-dioxopiperidin-3-yl)-3,5-difluorophenethyl)-3′,3′-difluoro-[1,4′-bipiperidin]-4-yl)-6-(2-hydroxypropan-2-yl)-2H-indazol-5-yl)pyrrolo[1,2-b]pyridazine-7-carboxamide

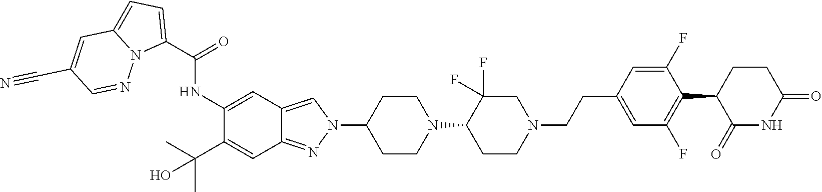 [SMILES: CC(C)(O)c1cc2nn(C3CCN([C@H]4CCN(CCc5cc(F)c([C@H]6CCC(=O)NC6=O)c(F)c5)CC4(F)F)CC3)cc2cc1NC(=O)c1ccc2cc(C#N)cnn12]

To a solution of (S)-3-cyano-N-(2-(3′,3′-difluoro-[1,4′-bipiperidin]-4-yl)-6-(2-hydroxypropan-2-yl)-2H-indazol-5-yl)pyrrolo[1,2-b]pyridazine-7-carboxamide (56 mg, 0.1 mmol) and (R)-2-(4-(2,6-dioxopiperidin-3-yl)-3,5-difluorophenyl)acetaldehyde (30 mg, 0.11 mmol) in DCM (5 mL) was added STAB (106 mg, 0.5 mmol) at room temperature. The resulting mixture was stirred for 2 hours at room temperature. The mixture was purified with Prep-TLC (DCM:MeOH=8:1) to afford the desired product (21 mg, 26%). ¹H NMR (500 MHz, DMSO) δ 12.03 (s, 1H), 10.94 (s, 1H), 8.93 (d, J=2.0 Hz, 1H), 8.74 (d, J=2.0 Hz, 1H), 8.56 (s, 1H), 8.38 (s, 1H), 7.72 (d, J=4.5 Hz, 1H), 7.58 (s, 1H), 7.09 (d, J=5.0 Hz, 1H), 7.04 (d, J=10.0 Hz, 2H), 5.73 (s, 1H), 4.49-4.39 (m, 1H), 4.20 (dd, J=12.5, 5.0 Hz, 1H), 3.18-3.06 (m, 3H), 3.04-2.98 (m, 1H), 2.96-2.86 (m, 1H), 2.81-2.73 (m, 4H), 2.67-2.60 (m, 2H), 2.57-2.52 (m, 2H), 2.37-2.25 (m, 1H), 2.21-2.03 (m, 6H), 2.02-1.95 (m, 1H), 1.87-1.73 (m, 2H), 1.63 (s, 6H). [M+H]⁺=814.6.

### Example 184: 3-cyano-N-(2-((1R,4r)-4-((R)-4-(1-(4-((R)-2,6-dioxopiperidin-3-yl)-3,5-difluorophenyl)azetidine-3-carbonyl)-2-(methoxymethyl)piperazin-1-yl)cyclohexyl)-6-(2-hydroxypropan-2-yl)-2H-indazol-5-yl)pyrrolo[1,2-b]pyridazine-7-carboxamide

### Step 1: methyl 2-((1R,4r)-4-((R)-4-(tert-butoxycarbonyl)-2-(methoxymethyl)piperazin-1-yl)cyclohexyl)-2H-indazole-6-carboxylate

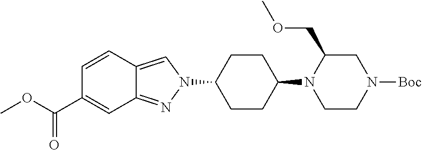 [SMILES: COC[C@H]1CN(C(=O)OC(C)(C)C)CCN1[C@H]1CC[C@H](n2cc3ccc(C(=O)OC)cc3n2)CC1]

To a solution of methyl 4-formyl-3-nitrobenzoate (3.2 g, 15.3 mmol) in propan-2-ol (100 mL) was added tert-butyl (R)-4-((1r,4R)-4-aminocyclohexyl)-3-(methoxymethyl)piperazine-1-carboxylate (5.0 g, 15.3 mmol). The mixture was stirred for 3 hours at 80° C. under N₂. To the above solution was added tributylphosphane (9.3 g, 46 mmol). The mixture was stirred overnight at 80° C. under N₂. The mixture was concentrated under vacuum to dryness and added PE (100 mL) stirred at room temperature for 2 hours filtered and evaporated in vacuum to afford product (5.9 g, 80%). [M+H]⁺=487.6.

### Step 2: methyl 2-((1R,4r)-4-((R)-2-(methoxymethyl)piperazin-1-yl)cyclohexyl)-5-nitro-2H-indazole-6-carboxylate

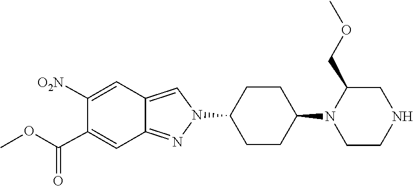 [SMILES: COC[C@H]1CNCCN1[C@H]1CC[C@H](n2cc3cc([N+](=O)[O-])c(C(=O)OC)cc3n2)CC1]

To a solution of methyl 2-((1R,4r)-4-((R)-4-(tert-butoxycarbonyl)-2-(methoxymethyl)piperazin-1-yl)cyclohexyl)-2H-indazole-6-carboxylate (5.9 g, 12.3 mmol) in conc.H₂SO₄ (60 mL) was slowed added NaNO₃ (2.12 g, 24.6 mmol) at 0° C. The mixture was stirred for 2 hours at 0° C. The mixture was quenched with ice-water, then adjusted to pH 9 with sat.Na₂CO₃ solution and extracted with DCM (50 mL×3). The combined organic layer was dried over anhydrous Na₂SO₄, filtered and evaporated in vacuum to afford product (5.8 g, crude), which was used for next step without further purification. [M+H]⁺=432.5.

### Step 3: methyl 2-((1R,4r)-4-((R)-4-(tert-butoxycarbonyl)-2-(methoxymethyl)piperazin-1-yl)cyclohexyl)-5-nitro-2H-indazole-6-carboxylate

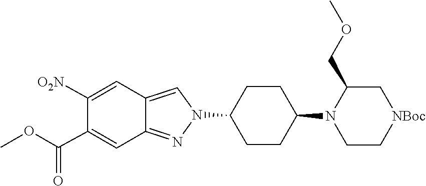 [SMILES: COC[C@H]1CN(C(=O)OC(C)(C)C)CCN1[C@H]1CC[C@H](n2cc3cc([N+](=O)[O-])c(C(=O)OC)cc3n2)CC1]

To a solution of methyl 2-((1R,4r)-4-((R)-2-(methoxymethyl)piperazin-1-yl)cyclohexyl)-5-nitro-2H-indazole-6-carboxylate (5.8 g, crude) in DCM (50 mL) was added DIPEA (3.5 g, 27 mmol) and Boc₂O (3.43 g, 16 mmol). The reaction mixture was stirred 18 hours at room temperature. The mixture was concentrated under vacuum to afford product (9 g, crude), which was used for next step without further purification. [M+H]⁺=532.5.

### Step 4: methyl 5-amino-2-((1R,4r)-4-((R)-4-(tert-butoxycarbonyl)-2-(methoxymethyl)piperazin-1-yl)cyclohexyl)-2H-indazole-6-carboxylate

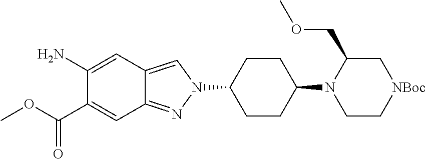 [SMILES: COC[C@H]1CN(C(=O)OC(C)(C)C)CCN1[C@H]1CC[C@H](n2cc3cc(N)c(C(=O)OC)cc3n2)CC1]

To a solution of methyl 2-((1R,4r)-4-((R)-4-(tert-butoxycarbonyl)-2-(methoxymethyl)piperazin-1-yl)cyclohexyl)-5-nitro-2H-indazole-6-carboxylate (9 g, crude) in MeOH (50 mL) and water (10 mL) was added NH₄Cl (7.2 g, 135 mmol) and Fe powder (3.8 g, 68 mmol) at room temperature. The resulting mixture was stirred for 2 hours at 70° C. The mixture was cooled to room temperature and concentrated under vacuum to afford product (11 g, crude), which was used for next step without further purification. [M+H]⁺=502.5.

### Step 5: methyl 2-((1R,4r)-4-((R)-4-(tert-butoxycarbonyl)-2-(methoxymethyl)piperazin-1-yl)cyclohexyl)-5-(2,2,2-trifluoroacetamido)-2H-indazole-6-carboxylate

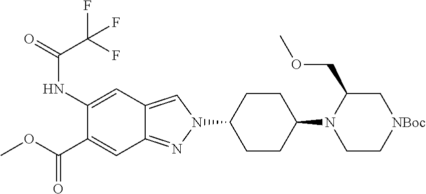 [SMILES: COC[C@H]1CN(C(=O)OC(C)(C)C)CCN1[C@H]1CC[C@H](n2cc3cc(NC(=O)C(F)(F)F)c(C(=O)OC)cc3n2)CC1]

To a solution of methyl 5-amino-2-((1R,4r)-4-((R)-4-(tert-butoxycarbonyl)-2-(methoxymethyl)piperazin-1-yl)cyclohexyl)-2H-indazole-6-carboxylate (11 g, crude) in DCM (50 mL) was added (CF₃CO)₂O (4.25 g, 20 mmol) at room temperature. The resulting mixture was stirred for 2 hours at room temperature. The mixture was concentrated under vacuum. The residue was purified by silica gel column chromatography (DCM:MeOH=30:1) to yield product (4.36 g, 66%). [M+H]⁺=598.4.

### Step 6: tert-butyl (R)-4-((1r,4R)-4-(6-(2-hydroxypropan-2-yl)-5-(2,2,2-trifluoroacetamido)-2H-indazol-2-yl)cyclohexyl)-3-(methoxymethyl)piperazine-1-carboxylate

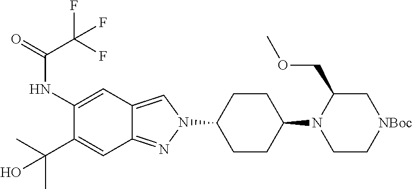 [SMILES: COC[C@H]1CN(C(=O)OC(C)(C)C)CCN1[C@H]1CC[C@H](n2cc3cc(NC(=O)C(F)(F)F)c(C(C)(C)O)cc3n2)CC1]

To a solution of methyl 2-((1R,4r)-4-((R)-4-(tert-butoxycarbonyl)-2-(methoxymethyl)piperazin-1-yl)cyclohexyl)-5-(2,2,2-trifluoroacetamido)-2H-indazole-6-carboxylate (4.36 g, 7.3 mmol) and LiCl (1.57 g, 36.5 mmol) in THF (40 mL) was added dropwise MeMgBr in THF (1 M, 75 mL, 75 mmol) at 0° C. The resulting mixture was stirred for 2 hours at 0° C. The mixture was quenched with sat.NH₄Cl solution and extracted with DCM. The combined organic layer was dried over anhydrous Na₂SO₄, filtered and evaporated under vacuum. The residue was purified on silica gel column chromatography (DCM:MeOH=10:1) to afford product (5.1 g, crude). [M+H]⁺=598.4.

### Step 7: tert-butyl (R)-4-((1r,4R)-4-(5-amino-6-(2-hydroxypropan-2-yl)-2H-indazol-2-yl)cyclohexyl)-3-(methoxymethyl)piperazine-1-carboxylate

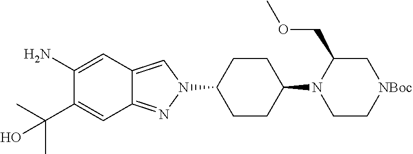 [SMILES: COC[C@H]1CN(C(=O)OC(C)(C)C)CCN1[C@H]1CC[C@H](n2cc3cc(N)c(C(C)(C)O)cc3n2)CC1]

To a solution of tert-butyl (R)-4-((1r,4R)-4-(6-(2-hydroxypropan-2-yl)-5-(2,2,2-trifluoroacetamido)-2H-indazol-2-yl)cyclohexyl)-3-(methoxymethyl)piperazine-1-carboxylate (5.1 g, 8.5 mmol) and NaOH (50 mL, 2N, 100 mmol) in EtOH (100 mL) was stirred for 2 hours at 80° C. The mixture was extracted with DCM (100 mL×3). The combined organic layer was dried over anhydrous Na₂SO₄, filtered and evaporated under vacuum. The residue was purified on silica gel column chromatography (DCM:MeOH=10:1) to afford product (1.87 g, 44%). [M+H]⁺=502.5.

### Step 8: tert-butyl (R)-4-((1r,4R)-4-(5-(3-cyanopyrrolo[1,2-b]pyridazine-7-carboxamido)-6-(2-hydroxypropan-2-yl)-2H-indazol-2-yl)cyclohexyl)-3-(methoxymethyl)piperazine-1-carboxylate

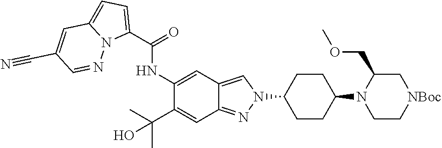 [SMILES: COC[C@H]1CN(C(=O)OC(C)(C)C)CCN1[C@H]1CC[C@H](n2cc3cc(NC(=O)c4ccc5cc(C#N)cnn45)c(C(C)(C)O)cc3n2)CC1]

To a solution of tert-butyl (R)-4-((1r,4R)-4-(5-amino-6-(2-hydroxypropan-2-yl)-2H-indazol-2-yl)cyclohexyl)-3-(methoxymethyl)piperazine-1-carboxylate (1.87 g, 3.7 mmol) and 3-cyanopyrrolo[1,2-b]pyridazine-7-carboxylic acid (0.7 g, 3.7 mmol) in DCM (25 mL) was added DIPEA (0.96 g, 7.4 mmol) and HATU (1.4 g, 3.7 mmol) at room temperature. The resulting mixture was stirred for 2 hours at room temperature. The mixture was evaporated under vacuum. The residue was purified with silica gel column chromatography (DCM:MeOH=100:1 to 90:10) to afford product (2.45 g, 98%). [M+H]⁺=691.7.

### Step 9: 3-cyano-N-(6-(2-hydroxypropan-2-yl)-2-((1R,4r)-4-((R)-2-(methoxymethyl)piperazin-1-yl)cyclohexyl)-2H-indazol-5-yl)pyrrolo[1,2-b]pyridazine-7-carboxamide

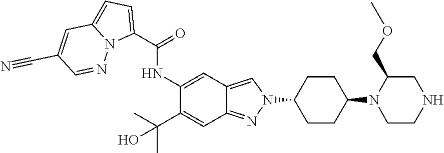 [SMILES: COC[C@H]1CNCCN1[C@H]1CC[C@H](n2cc3cc(NC(=O)c4ccc5cc(C#N)cnn45)c(C(C)(C)O)cc3n2)CC1]

To a solution of tert-butyl (R)-4-((1r,4R)-4-(5-(3-cyanopyrrolo[1,2-b]pyridazine-7-carboxamido)-6-(2-hydroxypropan-2-yl)-2H-indazol-2-yl)cyclohexyl)-3-(methoxymethyl)piperazine-1-carboxylate (2.45 g, 3.65 mmol) in DCM (20 mL) and MeOH (20 mL) was added conc.HCl (20 mL) at 0° C. The resulting mixture was stirred for 1 hour at 0° C. The mixture was quenched with sat.Na₂CO₃ solution and extracted with DCM (20 mL×3). The combined organic layer was dried over anhydrous Na₂SO₄, filtered and evaporated under vacuum to afford product (1.78 g, 85%). [M+H]⁺=571.4.

### Step 10: 3-cyano-N-(2-((1R,4r)-4-((R)-4-(1-(4-((R)-2,6-dioxopiperidin-3-yl)-3,5-difluorophenyl)azetidine-3-carbonyl)-2-(methoxymethyl)piperazin-1-yl)cyclohexyl)-6-(2-hydroxypropan-2-yl)-2H-indazol-5-yl)pyrrolo[1,2-b]pyridazine-7-carboxamide

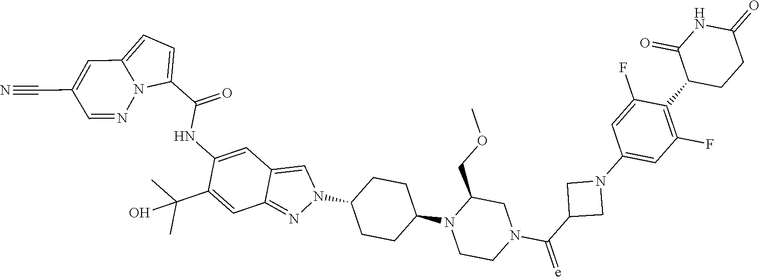

To a solution of 3-cyano-N-(6-(2-hydroxypropan-2-yl)-2-((1R,4r)-4-((R)-2-(methoxymethyl)piperazin-1-yl)cyclohexyl)-2H-indazol-5-yl)pyrrolo[1,2-b]pyridazine-7-carboxamide (57 mg, 0.1 mmol) and (R)-1-(4-(2,6-dioxopiperidin-3-yl)-3,5-difluorophenyl)azetidine-3-carboxylic acid (32 mg, 0.1 mmol) in DCM (5 mL) was added DIPEA (129 mg, 1 mmol) and T₃P in EA (50%, 128 mg, 0.2 mmol) at room temperature. The resulting mixture was stirred for 2 hours at room temperature. The mixture was added water (5 mL) and extracted with DCM (5 mL×3). The combined organic layer was dried over anhydrous Na₂SO₄, filtered and evaporated under vacuum. The residue was purified with Prep-HPLC (C-18 column chromatography (0.1% FA in water:acetonitrile=90:10˜60:40 gradient elution) to afford the desired product (30 mg, 34%). ¹H NMR (500 MHz, DMSO) δ 12.02 (s, 1H), 10.86 (s, 1H), 8.94 (d, J=2.0 Hz, 1H), 8.74 (d, J=2.0 Hz, 1H), 8.55 (s, 1H), 8.34 (d, J=2.5 Hz, 1H), 7.72 (d, J=4.5 Hz, 1H), 7.57 (s, 1H), 7.09 (d, J=5.0 Hz, 1H), 6.17 (dd, J=11.5, 4.0 Hz, 2H), 5.72 (s, 1H), 4.49-4.39 (m, 1H), 4.10-3.98 (m, 3H), 3.96-3.88 (m, 2H), 3.86-3.66 (m, 2H), 3.54-3.41 (m, 2H), 3.33 (s, 1H), 3.30-3.28 (m, 2H), 3.23-3.00 (m, 2H), 2.87-2.74 (m, 4H), 2.55-2.52 (m, 2H), 2.46 (s, 1H), 2.23-2.13 (m, 2H), 2.12-1.90 (m, 5H), 1.86-1.77 (m, 1H), 1.68-1.58 (m, 7H), 1.46-1.36 (m, 1H). [M+H]⁺=877.6.

### Example 321: 3-cyano-N-(2-((1R,4r)-4-((R)-4-(4-((R)-2,6-dioxopiperidin-3-yl)-3,5-difluorophenethyl)-2-(methoxymethyl)piperazin-1-yl)cyclohexyl)-6-(2-hydroxypropan-2-yl)-2H-indazol-5-yl)pyrrolo[1,2-b]pyridazine-7-carboxamide

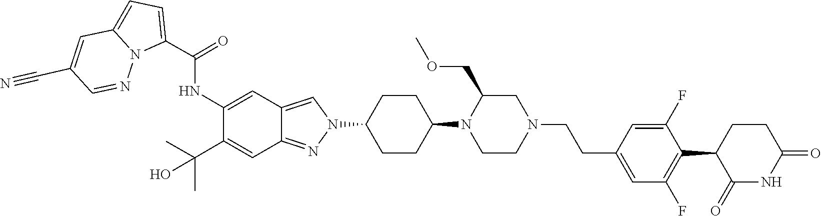 [SMILES: COC[C@H]1CN(CCc2cc(F)c([C@H]3CCC(=O)NC3=O)c(F)c2)CCN1[C@H]1CC[C@H](n2cc3cc(NC(=O)c4ccc5cc(C#N)cnn45)c(C(C)(C)O)cc3n2)CC1]

To a solution of 3-cyano-N-(6-(2-hydroxypropan-2-yl)-2-((1R,4r)-4-((R)-2-(methoxymethyl)piperazin-1-yl)cyclohexyl)-2H-indazol-5-yl)pyrrolo[1,2-b]pyridazine-7-carboxamide (57 mg, 0.1 mmol) and (R)-2-(4-(2,6-dioxopiperidin-3-yl)-3,5-difluorophenyl)acetaldehyde (27 mg, 0.1 mmol) in DCM (5 mL) was added STAB (106 mg, 0.5 mmol) at room temperature. The resulting mixture was stirred for 2 hours at room temperature. The mixture was added water (5 mL) and extracted with DCM (5 mL×3). The combined organic layer was dried over anhydrous Na₂SO₄, filtered and evaporated under vacuum. The residue was purified with Prep-HPLC (C-18 column chromatography (0.1% FA in water:acetonitrile=90:10˜60:40 gradient elution) to afford the desired product (34 mg, 41%). ¹H NMR (500 MHz, DMSO) δ 12.02 (s, 1H), 10.94 (s, 1H), 8.93 (d, J=2.0 Hz, 1H), 8.74 (d, J=2.0 Hz, 1H), 8.55 (s, 1H), 8.34 (s, 1H), 7.72 (d, J=5.0 Hz, 1H), 7.57 (s, 1H), 7.09 (d, J=5.0 Hz, 1H), 7.03 (d, J=10.0 Hz, 2H), 5.72 (s, 1H), 4.47-4.34 (m, 1H), 4.20 (dd, J=12.5, 5.0 Hz, 1H), 3.50 (dd, J=9.5, 4.5 Hz, 1H), 3.37-3.35 (m, 2H), 3.27 (s, 3H), 2.86-2.80 (m, 2H), 2.79-2.70 (m, 3H), 2.62-2.52 (m, 4H), 2.49-2.44 (m, 2H), 2.28-2.08 (m, 5H), 2.05-1.94 (m, 3H), 1.93-1.86 (m, 1H), 1.83-1.75 (m, 1H), 1.70-1.58 (m, 7H), 1.45-1.36 (m, 1H). [M+H]⁺=822.7.

### Example 322: 3-cyano-N-(2-((1R,4r)-4-((R)-4-((1-(4-((R)-2,6-dioxopiperidin-3-yl)-3,5-difluorophenyl)azetidin-3-yl)methyl)-2-(methoxymethyl)piperazin-1-yl)cyclohexyl)-6-(2-hydroxypropan-2-yl)-2H-indazol-5-yl)pyrrolo[1,2-b]pyridazine-7-carboxamide

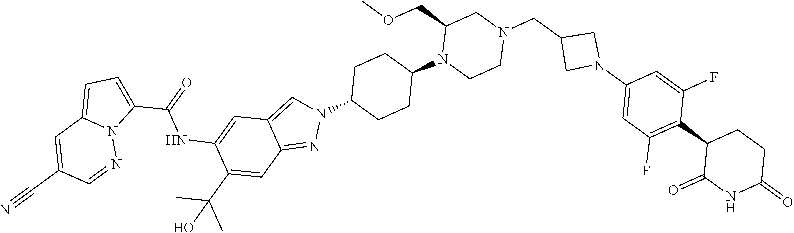 [SMILES: COC[C@H]1CN(CC2CN(c3cc(F)c([C@H]4CCC(=O)NC4=O)c(F)c3)C2)CCN1[C@H]1CC[C@H](n2cc3cc(NC(=O)c4ccc5cc(C#N)cnn45)c(C(C)(C)O)cc3n2)CC1]

The title compound (55 mg, 64%) was prepared in a manner similar to that described in Example 321.1H NMR (500 MHz, DMSO) δ 12.02 (s, 1H), 10.85 (s, 1H), 8.94 (d, J=2.0 Hz, 1H), 8.74 (d, J=2.0 Hz, 1H), 8.55 (s, 1H), 8.34 (s, 2H), 7.72 (d, 1H), 7.57 (s, 1H), 7.09 (d, 1H), 6.15-6.09 (m, 2H), 5.73 (s, 1H), 4.47-4.38 (m, 1H), 4.02 (dd, J=12.5, 5.0 Hz, 1H), 3.92 (dd, J=13.5, 7.5 Hz, 2H), 3.57-3.44 (m, 4H), 3.28 (s, 3H), 2.94-2.87 (m, 1H), 2.84-2.77 (m, 2H), 2.75-2.71 (m, 1H), 2.62-2.54 (m, 5H), 2.47-2.41 (m, 1H), 2.20-2.13 (m, 3H), 2.11-2.04 (m, 2H), 2.02-1.88 (m, 4H), 1.80-1.74 (m, 1H), 1.69-1.60 (m, 7H), 1.46-1.34 (m, 1H). [M+H]⁺=863.7.

This compound could also be synthesized in another manner.

### Step 1: methyl 2-((1R,4r)-4-((R)-4-(tert-butoxycarbonyl)-2-(methoxymethyl)piperazin-1-yl)cyclohexyl)-2H-indazole-6-carboxylate

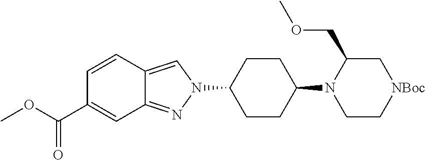 [SMILES: COC[C@H]1CN(C(=O)OC(C)(C)C)CCN1[C@H]1CC[C@H](n2cc3ccc(C(=O)OC)cc3n2)CC1]

To a solution of methyl 4-formyl-3-nitrobenzoate (33.2 g, 158 mmol) in propan-2-ol (500 mL) was added tert-butyl (R)-4-((1r,4R)-4-aminocyclohexyl)-3-(methoxymethyl)piperazine-1-carboxylate (50 g, 158 mmol). The mixture was stirred for 4 hours at 80° C. under N₂. To the above solution was added tri-n-butylphosphane (95.3 g, 472 mmol). The mixture was stirred for 18 hours at 80° C. under N₂. The mixture was cooled to room temperature and concentrated under vacuum. The residue was slurried with petroleum ether (800 mL) and filtered. The solid was filtered and dried under vacuum to afford product (55 g, 71.4%). [M+H]⁺=487.4.

### Step 2: methyl 2-((1R,4r)-4-((R)-4-(tert-butoxycarbonyl)-2-(methoxymethyl)piperazin-1-yl)cyclohexyl)-5-nitro-2H-indazole-6-carboxylate

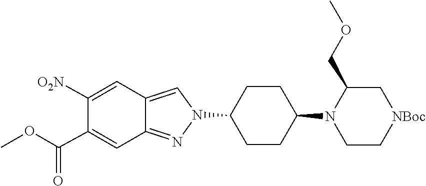 [SMILES: COC[C@H]1CN(C(=O)OC(C)(C)C)CCN1[C@H]1CC[C@H](n2cc3cc([N+](=O)[O-])c(C(=O)OC)cc3n2)CC1]

To a solution of methyl 2-((1R,4r)-4-((R)-4-(tert-butoxycarbonyl)-2-(methoxymethyl)piperazin-1-yl)cyclohexyl)-2H-indazole-6-carboxylate (55 g, 113 mmol) in conc. H₂SO₄ (200 mL) was slowed added NaNO₃ (15 g, 174 mmol) batchwise at 0° C. The mixture was stirred for 3 hours at 0° C. The mixture was quenched with ice-water, then adjusted to pH 8 with sat. Na₂CO₃ solution then added DCM (500 mL), DIPEA (29.2 g, 226 mmol) and Boc₂O (24.6 g, 113 mmol). The mixture was stirred for 18 hours at room temperature and extracted with DCM (500 mL×3). The combined organic layer was dried over anhydrous Na₂SO₄, filtered and evaporated in vacuum to afford product (58 g, 96.4%), which was used next step without further purification. [M+H]⁺=532.5.

### Step 3: methyl 5-amino-2-((1R,4r)-4-((R)-4-(tert-butoxycarbonyl)-2-(methoxymethyl)piperazin-1-yl)cyclohexyl)-2H-indazole-6-carboxylate

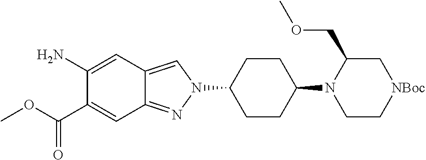 [SMILES: COC[C@H]1CN(C(=O)OC(C)(C)C)CCN1[C@H]1CC[C@H](n2cc3cc(N)c(C(=O)OC)cc3n2)CC1]

To a solution of methyl 2-((1R,4r)-4-((R)-4-(tert-butoxycarbonyl)-2-(methoxymethyl)piperazin-1-yl)cyclohexyl)-5-nitro-2H-indazole-6-carboxylate (58 g, 109 mmol) in MeOH (100 mL) and THF (100 mL) was added Pd/C (5.8 g, 10% weight) under H₂. The solution was stirred at room temperature for 24 hours, filtered and evaporated in vacuum to afford product (55 g, 99.9%), which was used next step without further purification. [M+H]⁺=502.5.

### Step 4: methyl 2-((1R,4r)-4-((R)-4-(tert-butoxycarbonyl)-2-(methoxymethyl)piperazin-1-yl)cyclohexyl)-5-(2,2,2-trifluoroacetamido)-2H-indazole-6-carboxylate

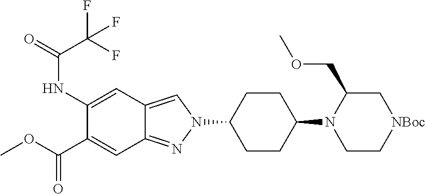 [SMILES: COC[C@H]1CN(C(=O)OC(C)(C)C)CCN1[C@H]1CC[C@H](n2cc3cc(NC(=O)C(F)(F)F)c(C(=O)OC)cc3n2)CC1]

To a solution of methyl 5-amino-2-((1R,4r)-4-((R)-4-(tert-butoxycarbonyl)-2-(methoxymethyl)piperazin-1-yl)cyclohexyl)-2H-indazole-6-carboxylate (55 g, 109 mmol) in DCM (500 mL) was slowed added DIPEA (16.9 g, 130 mmol) and (CF₃CO)₂O (25.2 g, 120 mmol). The mixture was stirred for 2 hours at room temperature and washed with sat. NaHCO₃ (500 mL) and brined (500 mL). The organic layer was dried over anhydrous Na₂SO₄, filtered and evaporated in vacuum to afford product (70 g, crude), which was used without further purification. [M+H]⁺=598.5.

### Step 5: tert-butyl (R)-4-((1r,4R)-4-(6-(2-hydroxypropan-2-yl)-5-(2,2,2-trifluoroacetamido)-2H-indazol-2-yl)cyclohexyl)-3-(methoxymethyl)piperazine-1-carboxylate

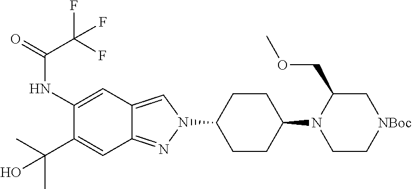 [SMILES: COC[C@H]1CN(C(=O)OC(C)(C)C)CCN1[C@H]1CC[C@H](n2cc3cc(NC(=O)C(F)(F)F)c(C(C)(C)O)cc3n2)CC1]

To a solution of methyl 2-((1R,4r)-4-((R)-4-(tert-butoxycarbonyl)-2-(methoxymethyl)piperazin-1-yl)cyclohexyl)-5-(2,2,2-trifluoroacetamido)-2H-indazole-6-carboxylate (70 g, 109 mmol) and LiCl (23 g, 547 mmol) in THF (800 mL) was added dropwise MeMgBr in MeTHF (3M, 184 mL, 552 mmol) at 0° C. The resulting mixture was stirred for 2 hours at 0° C. The mixture was quenched with sat. NH₄Cl solution and extracted with DCM (3×500 mL). The combined organic layer was washed with brined (500 mL) and dried over anhydrous Na₂SO₄, filtered and concentrated under vacuum. The residue was purified on silica gel column chromatography (PE:EA=100:1 to 1:100) to afford product (45 g, 75.4% two step). [M+H]⁺=598.5.

### Step 6: tert-butyl (R)-4-((1r,4R)-4-(5-amino-6-(2-hydroxypropan-2-yl)-2H-indazol-2-yl)cyclohexyl)-3-(methoxymethyl)piperazine-1-carboxylate

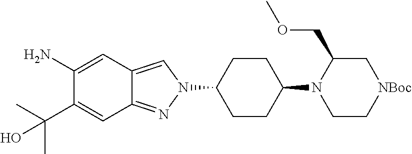 [SMILES: COC[C@H]1CN(C(=O)OC(C)(C)C)CCN1[C@H]1CC[C@H](n2cc3cc(N)c(C(C)(C)O)cc3n2)CC1]

To a solution of tert-butyl (R)-4-((1r,4R)-4-(6-(2-hydroxypropan-2-yl)-5-(2,2,2-trifluoroacetamido)-2H-indazol-2-yl)cyclohexyl)-3-(methoxymethyl)piperazine-1-carboxylate (45 g, 75 mmol) in MeOH (360 mL) was added NaOH aqueous solution (2 M, 360 mL, 720 mmol). The mixture was stirred at room temperature for 2 hours. Then the mixture was adjusted to pH 7 with HCl 6 M then extracted with DCM (500 mL×3). The combined organic layer was dried over anhydrous Na₂SO₄, filtered and concentrated under vacuum to afford product (34.5 g, 91.6%), which was used next step without further purification. [M+H]⁺=502.4.

### Step 7: tert-butyl (R)-4-((1r,4R)-4-(5-(3-cyanopyrrolo[1,2-b]pyridazine-7-carboxamido)-6-(2-hydroxypropan-2-yl)-2H-indazol-2-yl)cyclohexyl)-3-(methoxymethyl)piperazine-1-carboxylate

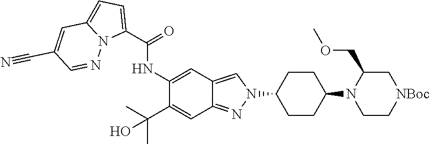 [SMILES: COC[C@H]1CN(C(=O)OC(C)(C)C)CCN1[C@H]1CC[C@H](n2cc3cc(NC(=O)c4ccc5cc(C#N)cnn45)c(C(C)(C)O)cc3n2)CC1]

To a stirred solution of 3-cyanopyrrolo[1,2-b]pyridazine-7-carboxylic acid (12.6 g, 67 mmol) and tert-butyl (R)-4-((1r,4R)-4-(5-amino-6-(2-hydroxypropan-2-yl)-2H-indazol-2-yl)cyclohexyl)-3-(methoxymethyl)piperazine-1-carboxylate (33.6 g, 67 mmol) in DCM (325 mL) was added HATU (25.5 g, 67 mmol) and DIPEA (17.3 g, 134 mmol). The solution was stirred at room temperature for 3 hours. The solution was added water (500 mL) and extracted with DCM (500 mL×3). The combined organic layer was dried over Na₂SO₄ and concentrated under vacuum. The combined organic layer was concentrated under vacuum and purified on silica gel column chromatography (DCM:MeOH=90:10) to afford product (45 g, 100%). [M+H]⁺=671.7.

### Step 8: 3-cyano-N-(6-(2-hydroxypropan-2-yl)-2-((1R,4r)-4-((R)-2-(methoxymethyl)piperazin-1-yl)cyclohexyl)-2H-indazol-5-yl)pyrrolo[1,2-b]pyridazine-7-carboxamide

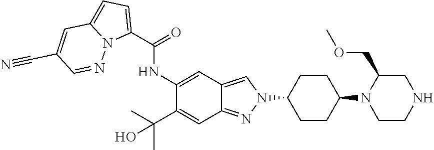 [SMILES: COC[C@H]1CNCCN1[C@H]1CC[C@H](n2cc3cc(NC(=O)c4ccc5cc(C#N)cnn45)c(C(C)(C)O)cc3n2)CC1]

To a stirring solution of tert-butyl (R)-4-((1r,4R)-4-(5-(3-cyanopyrrolo[1,2-b]pyridazine-7-carboxamido)-6-(2-hydroxypropan-2-yl)-2H-indazol-2-yl)cyclohexyl)-3-(methoxymethyl)piperazine-1-carboxylate (45 g, 67 mmol) in MeOH (400 mL) and DCM (400 mL) was added 12 M HCl (400 mL). The reaction mixture was stirred for 2 hours at room temperature, adjusted to pH 7 with sat. NaHCO₃ and extracted with DCM (500 mL×3). The combined organic layer was dried over Na₂SO₄ and concentrated under vacuum. The combined organic layer was concentrated to dryness to afford product (34 g, 88.8%). [M+H]⁺=571.7.

### Step 9: 3-cyano-N-(2-((1R,4r)-4-((R)-4-((1-(4-((R)-2,6-dioxopiperidin-3-yl)-3,5-difluorophenyl)azetidin-3-yl)methyl)-2-(methoxymethyl)piperazin-1-yl)cyclohexyl)-6-(2-hydroxypropan-2-yl)-2H-indazol-5-yl)pyrrolo[1,2-b]pyridazine-7-carboxamide

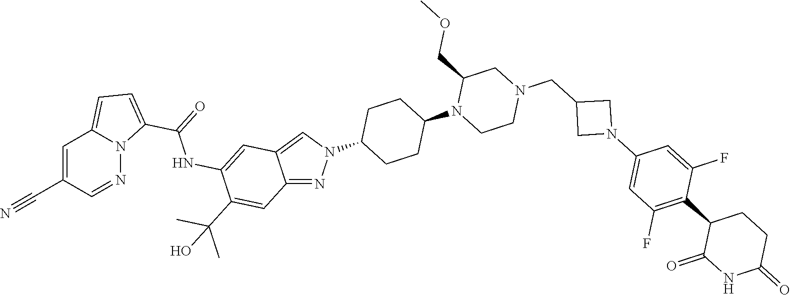 [SMILES: COC[C@H]1CN(CC2CN(c3cc(F)c([C@H]4CCC(=O)NC4=O)c(F)c3)C2)CCN1[C@H]1CC[C@H](n2cc3cc(NC(=O)c4ccc5cc(C#N)cnn45)c(C(C)(C)O)cc3n2)CC1]

A solution of 3-cyano-N-(6-(2-hydroxypropan-2-yl)-2-((1R,4r)-4-((R)-2-(methoxymethyl)piperazin-1-yl)cyclohexyl)-2H-indazol-5-yl)pyrrolo[1,2-b]pyridazine-7-carboxamide (4 g, 7 mmol) and (R)-1-(4-(2,6-dioxopiperidin-3-yl)-3,5-difluorophenyl)azetidine-3-carbaldehyde (CAS: 2873368-99-9, which was obtained in the same way described in WO2022012622A1) (2.2 g, 7 mmol) in DCM (80 mL) was stirred at room temperature for 1 hour, then NaBH(OAc)₃ (3.0 g, 14 mmol) was added portion wise. The solution was stirred at room temperature for 4 hours. The mixture was concentrated under vacuum and purified on silica gel column chromatography (DCM:MeOH=90:10) to afford product (4.0 g, 66.3%). ¹H NMR (500 MHz, DMSO) δ 11.95 (s, 1H), 10.78 (s, 1H), 8.87 (d, J=2.5 Hz, 1H), 8.67 (d, J=2.0 Hz, 1H), 8.52-8.46 (m, 1H), 8.27 (s, 1H), 8.07 (s, 1H), 7.65 (d, J=4.5 Hz, 1H), 7.50 (s, 1H), 7.02 (d, J=4.5 Hz, 1H), 6.05 (s, 1H), 6.03 (s, 1H), 5.73-5.57 (m, 1H), 4.40-4.32 (m, 1H), 3.96 (dd, J=12.5, 5.0 Hz, 1H), 3.86 (dd, J=12.5, 7.5 Hz, 2H), 3.47-3.39 (m, 3H), 3.24-3.20 (m, 4H), 2.92-2.79 (m, 3H), 2.76-2.67 (m, 2H), 2.64-2.45 (m, 6H), 2.42-2.37 (m, 1H), 2.17-2.05 (m, 4H), 2.02-1.85 (m, 5H), 1.76-1.70 (m, 1H), 1.61-1.54 (m, 7H), 1.42-1.32 (m, 1H). [M+H]⁺=863.6.

### Example 323: 3-cyano-N-(2-((1R,4r)-4-((R)-4-(1-(4-((R)-2,6-dioxopiperidin-3-yl)-3,5-difluorophenyl)azetidin-3-yl)-2-(methoxymethyl)piperazin-1-yl)cyclohexyl)-6-(2-hydroxypropan-2-yl)-2H-indazol-5-yl)pyrrolo[1,2-b]pyridazine-7-carboxamide

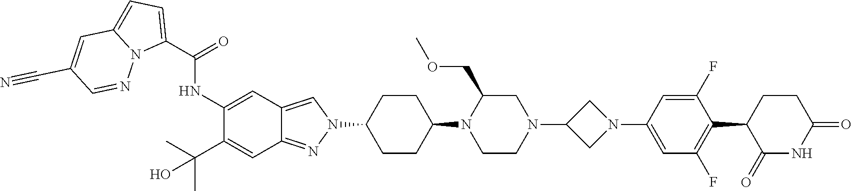 [SMILES: COC[C@H]1CN(C2CN(c3cc(F)c([C@H]4CCC(=O)NC4=O)c(F)c3)C2)CCN1[C@H]1CC[C@H](n2cc3cc(NC(=O)c4ccc5cc(C#N)cnn45)c(C(C)(C)O)cc3n2)CC1]

The title compound (8 mg, 9%) was prepared in a manner similar to that described in Example 321. ¹H NMR (500 MHz, DMSO) δ 12.02 (s, 1H), 10.85 (s, 1H), 8.93 (s, 1H), 8.74 (s, 1H), 8.55 (s, 1H), 8.33 (s, 1H), 7.72 (d, J=5.0 Hz, 1H), 7.57 (s, 1H), 7.09 (d, J=4.5 Hz, 1H), 6.16-6.08 (m, 2H), 5.72 (s, 1H), 4.45-4.39 (m, 1H), 4.08-4.00 (m, 1H), 3.91 (t, J=7.5 Hz, 2H), 3.66-3.58 (m, 2H), 3.55-3.51 (m, 1H), 3.38-3.34 (m, 1H), 3.28 (s, 3H), 3.25-3.21 (m, 1H), 2.88-2.74 (m, 4H), 2.61-2.55 (m, 1H), 2.48-2.43 (m, 2H), 2.21-2.08 (m, 4H), 2.04-1.91 (m, 5H), 1.80-1.75 (m, 1H), 1.68-1.61 (m, 7H), 1.43-1.38 (m, 1H), 1.24-1.23 (m, 1H). [M+H]⁺=849.7.

### Example 324: 3-cyano-N-(2-((1R,4r)-4-((R)-4-(1-(4-((R)-2,6-dioxopiperidin-3-yl)-3,5-difluorophenyl)piperidin-4-yl)-2-(methoxymethyl)piperazin-1-yl)cyclohexyl)-6-(2-hydroxypropan-2-yl)-2H-indazol-5-yl)pyrrolo[1,2-b]pyridazine-7-carboxamide

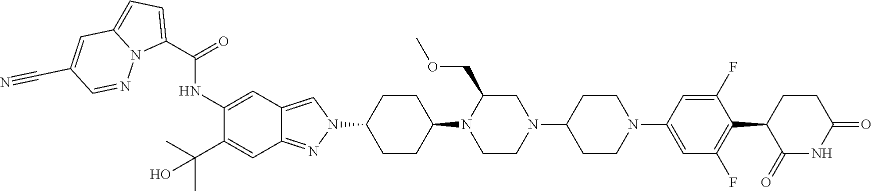 [SMILES: COC[C@H]1CN(C2CCN(c3cc(F)c([C@H]4CCC(=O)NC4=O)c(F)c3)CC2)CCN1[C@H]1CC[C@H](n2cc3cc(NC(=O)c4ccc5cc(C#N)cnn45)c(C(C)(C)O)cc3n2)CC1]

The title compound (12 mg, 14%) was prepared in a manner similar to that described in Example 321. ¹H NMR (500 MHz, DMSO) δ 12.02 (s, 1H), 10.87 (s, 1H), 8.93 (d, J=2.0 Hz, 1H), 8.74 (d, J=2.0 Hz, 1H), 8.55 (s, 1H), 8.33 (s, 1H), 7.72 (d, J=4.5 Hz, 1H), 7.58 (s, 1H), 7.09 (d, J=5.0 Hz, 1H), 6.67-6.57 (m, 2H), 5.72 (s, 1H), 4.41 (s, 1H), 4.04 (dd, J=12.0, 4.5 Hz, 1H), 3.81-3.72 (m, 2H), 3.53-3.47 (m, 1H), 3.36-3.33 (m, 2H), 3.27 (s, 3H), 2.84-2.72 (m, 6H), 2.47-2.40 (m, 1H), 2.36-2.24 (m, 2H), 2.21-2.12 (m, 3H), 2.11-2.05 (m, 1H), 2.01-1.93 (m, 3H), 1.92-1.87 (m, 1H), 1.84-1.74 (m, 3H), 1.69-1.61 (m, 7H), 1.48-1.37 (m, 3H), 1.28-1.21 (m, 2H). [M+H]⁺=877.8.

### Example 304: 3-cyano-N-(2-((1S,4r)-4-((S)-4-(1-(4-((R)-2,6-dioxopiperidin-3-yl)-3,5-difluorophenyl)azetidine-3-carbonyl)-2-methylpiperazin-1-yl)cyclohexyl)-6-(2-hydroxypropan-2-yl)-2H-indazol-5-yl)pyrrolo[1,2-b]pyridazine-7-carboxamide

### Step 1: tert-butyl (S)-4-((1r,4S)-4-(5-amino-6-(2-hydroxypropan-2-yl)-2H-indazol-2-yl)cyclohexyl)-3-methylpiperazine-1-carboxylate

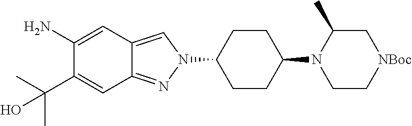 [SMILES: C[C@H]1CN(C(=O)OC(C)(C)C)CCN1[C@H]1CC[C@H](n2cc3cc(N)c(C(C)(C)O)cc3n2)CC1]

To a solution of tert-butyl (S)-4-((1r,4S)-4-(6-(2-hydroxypropan-2-yl)-5-(2,2,2-trifluoroacetamido)-2H-indazol-2-yl)cyclohexyl)-3-methylpiperazine-1-carboxylate (0.56 g, 1.0 mmol) and NaOH (20 mL, 2N, 40 mmol) in EtOH (40 mL) was stirred for 2 hours at 80° C. The mixture was extracted with DCM (40 mL×3). The combined organic layer was dried over anhydrous Na₂SO₄, filtered and evaporated under vacuum to give product (0.47 g, 98%). [M+H]⁺=472.4.

### Step 2: tert-butyl (S)-4-((1r,4S)-4-(5-(3-cyanopyrrolo[1,2-b]pyridazine-7-carboxamido)-6-(2-hydroxypropan-2-yl)-2H-indazol-2-yl)cyclohexyl)-3-methylpiperazine-1-carboxylate

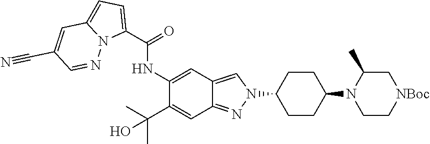 [SMILES: C[C@H]1CN(C(=O)OC(C)(C)C)CCN1[C@H]1CC[C@H](n2cc3cc(NC(=O)c4ccc5cc(C#N)cnn45)c(C(C)(C)O)cc3n2)CC1]

To a solution of tert-butyl (S)-4-((1r,4S)-4-(5-amino-6-(2-hydroxypropan-2-yl)-2H-indazol-2-yl)cyclohexyl)-3-methylpiperazine-1-carboxylate (0.46 g, 0.97 mmol) and 3-cyanopyrrolo[1,2-b]pyridazine-7-carboxylic acid (0.19 g, 1.0 mmol) in DCM (15 mL) and DMF (10 mL) was added DIPEA (0.26 g, 2.0 mmol) and HATU (0.38 g, 1.0 mmol) at room temperature. The resulting mixture was stirred for 18 hours at room temperature. The mixture was evaporated under vacuum. The residue was purified with silica gel column chromatography (DCM:MeOH=100:1 to 90:10) to afford product (0.63 g, 98%). [M+H]⁺=641.7.

### Step 3: 3-cyano-N-(6-(2-hydroxypropan-2-yl)-2-((1S,4r)-4-((S)-2-methylpiperazin-1-yl)cyclohexyl)-2H-indazol-5-yl)pyrrolo[1,2-b]pyridazine-7-carboxamide

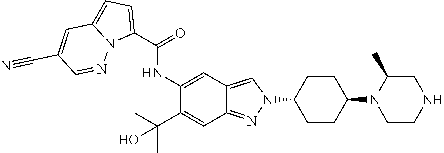 [SMILES: C[C@H]1CNCCN1[C@H]1CC[C@H](n2cc3cc(NC(=O)c4ccc5cc(C#N)cnn45)c(C(C)(C)O)cc3n2)CC1]

To a solution of tert-butyl (S)-4-((1r,4S)-4-(5-(3-cyanopyrrolo[1,2-b]pyridazine-7-carboxamido)-6-(2-hydroxypropan-2-yl)-2H-indazol-2-yl)cyclohexyl)-3-methylpiperazine-1-carboxylate (0.63 g, 1.0 mmol) in DCM (10 mL) and MeOH (10 mL) was added conc.HCl (10 mL) at 0° C. The resulting mixture was stirred for lhour at 0° C. The mixture was added sat.Na₂CO₃ solution to make pH to 6-7 and extracted with DCM (20 mL×3). The combined organic layer was dried over anhydrous Na₂SO₄, filtered and evaporated under vacuum to afford product (0.56 g, crude). [M+H]⁺=541.7.

### Step 4: 3-cyano-N-(2-((1S,4r)-4-((S)-4-(1-(4-((R)-2,6-dioxopiperidin-3-yl)-3,5-difluorophenyl)azetidine-3-carbonyl)-2-methylpiperazin-1-yl)cyclohexyl)-6-(2-hydroxypropan-2-yl)-2H-indazol-5-yl)pyrrolo[1,2-b]pyridazine-7-carboxamide

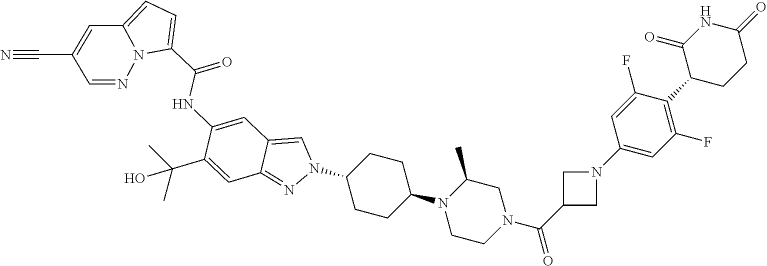 [SMILES: C[C@H]1CN(C(=O)C2CN(c3cc(F)c([C@H]4CCC(=O)NC4=O)c(F)c3)C2)CCN1[C@H]1CC[C@H](n2cc3cc(NC(=O)c4ccc5cc(C#N)cnn45)c(C(C)(C)O)cc3n2)CC1]

To a solution of 3-cyano-N-(6-(2-hydroxypropan-2-yl)-2-((1S,4r)-4-((S)-2-methylpiperazin-1-yl)cyclohexyl)-2H-indazol-5-yl)pyrrolo[1,2-b]pyridazine-7-carboxamide (54 mg, 0.1 mmol) and (R)-1-(4-(2,6-dioxopiperidin-3-yl)-3,5-difluorophenyl)azetidine-3-carboxylic acid (32 mg, 0.1 mmol) in DCM (5 mL) was added DIPEA (129 mg, 1 mmol) and T₃P in EA (50%, 128 mg, 0.2 mmol) at room temperature. The resulting mixture was stirred for 2 hours at room temperature. The mixture was added water (5 mL) and extracted with DCM (5 mL×3). The combined organic layer was dried over anhydrous Na₂SO₄, filtered and evaporated under vacuum. The residue was purified with Prep-HPLC (C-18 column chromatography (0.1% FA in water:acetonitrile=90:10 to 60:40 gradient elution) to afford the desired product (30 mg, 35%). ¹H NMR (500 MHz, DMSO) δ 12.02 (s, 1H), 10.86 (s, 1H), 8.94 (d, J=2.0 Hz, 1H), 8.74 (d, J=2.0 Hz, 1H), 8.55 (s, 1H), 8.34 (d, J=2.5 Hz, 1H), 7.72 (d, J=4.5 Hz, 1H), 7.57 (s, 1H), 7.09 (d, J=5.0 Hz, 1H), 6.17 (d, J=13.0 Hz, 2H), 5.72 (s, 1H), 4.48-4.38 (m, 1H), 4.09-4.00 (m, 3H), 3.95-3.88 (m, 2H), 3.88-3.79 (m, 1H), 3.51-3.40 (m, 1H), 3.30-3.08 (m, 1H), 2.93-2.72 (m, 4H), 2.67-2.62 (m, 1H), 2.36-2.25 (m, 1H), 2.22-2.14 (m, 2H), 2.10-2.00 (m, 2H), 1.97-1.91 (m, 2H), 1.90-1.84 (m, 1H), 1.79-1.69 (m, 2H), 1.63 (s, 6H), 1.42-1.39 (m, 1H), 1.25-1.22 (m, 2H), 1.07-1.03 (m, 3H). [M+H]⁺=847.7.

### Example 305: 3-cyano-N-(2-((1S,4r)-4-((S)-4-(4-((R)-2,6-dioxopiperidin-3-yl)-3,5-difluorophenethyl)-2-methylpiperazin-1-yl)cyclohexyl)-6-(2-hydroxypropan-2-yl)-2H-indazol-5-yl)pyrrolo[1,2-b]pyridazine-7-carboxamide

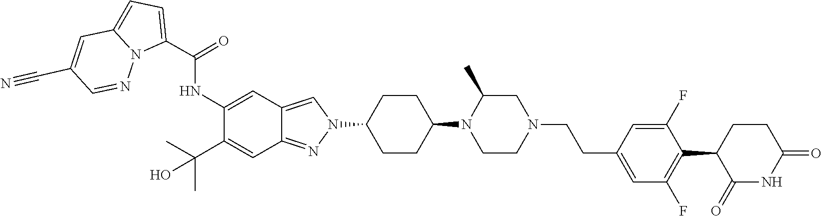 [SMILES: C[C@H]1CN(CCc2cc(F)c([C@H]3CCC(=O)NC3=O)c(F)c2)CCN1[C@H]1CC[C@H](n2cc3cc(NC(=O)c4ccc5cc(C#N)cnn45)c(C(C)(C)O)cc3n2)CC1]

To a solution of 3-cyano-N-(6-(2-hydroxypropan-2-yl)-2-((1S,4r)-4-((S)-2-methylpiperazin-1-yl)cyclohexyl)-2H-indazol-5-yl)pyrrolo[1,2-b]pyridazine-7-carboxamide (54 mg, 0.1 mmol) and (R)-2-(4-(2,6-dioxopiperidin-3-yl)-3,5-difluorophenyl)acetaldehyde (27 mg, 0.1 mmol) in DCM (5 mL) was added STAB (106 mg, 0.5 mmol) at room temperature. The resulting mixture was stirred for 2 hours at room temperature. The mixture was added water (5 mL) and extracted with DCM (5 mL×3). The combined organic layer was dried over anhydrous Na₂SO₄, filtered and evaporated under vacuum. The residue was purified with Prep-TLC (DCM:MeOH=9:1) to afford the desired product (21 mg, 26%). ¹H NMR (500 MHz, DMSO) δ 12.02 (s, 1H), 10.94 (s, 1H), 8.93 (d, J=2.0 Hz, 1H), 8.74 (d, J=2.0 Hz, 1H), 8.55 (s, 1H), 8.34 (s, 1H), 7.72 (d, J=5.0 Hz, 1H), 7.57 (s, 1H), 7.09 (d, J=5.0 Hz, 1H), 7.03 (d, J=10.0 Hz, 2H), 5.72 (s, 1H), 4.46-4.35 (m, 1H), 4.20 (dd, J=13.0, 5.0 Hz, 1H), 3.31-3.28 (m, 2H), 2.88-2.68 (m, 7H), 2.57-2.52 (m, 1H), 2.47-2.38 (m, 2H), 2.21-2.08 (m, 4H), 2.03-1.96 (m, 2H), 1.94-1.86 (m, 2H), 1.79-1.69 (m, 2H), 1.63 (s, 6H), 1.43-1.35 (m, 1H), 1.25-1.22 (m, 1H), 1.07-0.97 (m, 3H). [M+H]⁺=792.7.

### Example 306: 3-cyano-N-(2-((1S,4r)-4-((S)-4-((1-(4-((R)-2,6-dioxopiperidin-3-yl)-3,5-difluorophenyl)azetidin-3-yl)methyl)-2-methylpiperazin-1-yl)cyclohexyl)-6-(2-hydroxypropan-2-yl)-2H-indazol-5-yl)pyrrolo[1,2-b]pyridazine-7-carboxamide

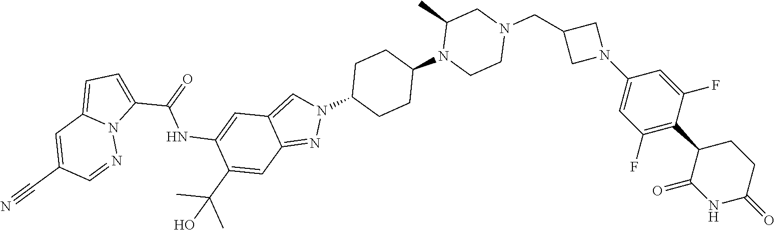 [SMILES: C[C@H]1CN(CC2CN(c3cc(F)c([C@H]4CCC(=O)NC4=O)c(F)c3)C2)CCN1[C@H]1CC[C@H](n2cc3cc(NC(=O)c4ccc5cc(C#N)cnn45)c(C(C)(C)O)cc3n2)CC1]

The title compound (25 mg, 31%) was prepared in a manner similar to that described in Example 305. ¹H NMR (500 MHz, DMSO) δ 12.02 (s, 1H), 10.85 (s, 1H), 8.94 (d, J=2.0 Hz, 1H), 8.74 (d, J=2.0 Hz, 1H), 8.55 (s, 1H), 8.33 (s, 1H), 7.72 (d, J=4.5 Hz, 1H), 7.57 (s, 1H), 7.09 (d, J=5.0 Hz, 1H), 6.11 (d, J=11.0 Hz, 2H), 5.72 (s, 1H), 4.46-4.36 (m, 1H), 4.02 (dd, J=12.5, 5.0 Hz, 1H), 3.93 (t, J=6.5 Hz, 2H), 3.50-3.44 (m, 2H), 3.31-3.27 (m, 2H), 2.96-2.84 (m, 2H), 2.81-2.73 (m, 2H), 2.71-2.58 (m, 3H), 2.44-2.37 (m, 1H), 2.21-2.14 (m, 2H), 2.13-1.99 (m, 3H), 1.97-1.83 (m, 4H), 1.77-1.69 (m, 2H), 1.63 (s, 6H), 1.45-1.35 (m, 1H), 1.27-1.21 (m, 1H), 1.02 (d, J=6.0 Hz, 3H). [M+H]⁺=833.7.

### Example 308: 3-cyano-N-(2-((1S,4r)-4-((S)-4-(1-(4-((R)-2,6-dioxopiperidin-3-yl)-3,5-difluorophenyl)azetidin-3-yl)-2-methylpiperazin-1-yl)cyclohexyl)-6-(2-hydroxypropan-2-yl)-2H-indazol-5-yl)pyrrolo[1,2-b]pyridazine-7-carboxamide

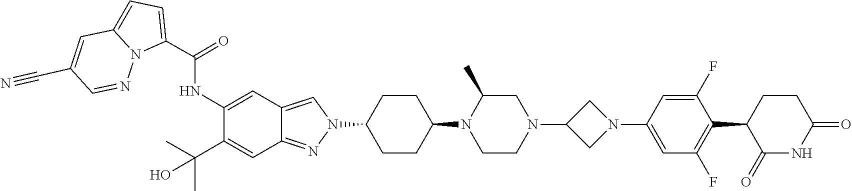 [SMILES: C[C@H]1CN(C2CN(c3cc(F)c([C@H]4CCC(=O)NC4=O)c(F)c3)C2)CCN1[C@H]1CC[C@H](n2cc3cc(NC(=O)c4ccc5cc(C#N)cnn45)c(C(C)(C)O)cc3n2)CC1]

The title compound (23 mg, 28%) was prepared in a manner similar to that described in Example 305. ¹H NMR (500 MHz, DMSO) δ 12.02 (s, 1H), 10.86 (s, 1H), 8.94 (d, J=2.0 Hz, 1H), 8.74 (d, J=2.0 Hz, 1H), 8.55 (s, 1H), 8.33 (s, 1H), 7.72 (d, J=4.5 Hz, 1H), 7.56 (s, 1H), 7.09 (d, J=5.0 Hz, 1H), 6.12 (d, J=11.5 Hz, 2H), 5.72 (s, 1H), 4.48-4.38 (m, 1H), 4.03 (dd, J=12.5, 5.0 Hz, 1H), 3.95-3.86 (m, 2H), 3.69-3.60 (m, 2H), 3.31-3.28 (m, 2H), 3.27-3.17 (m, 1H), 2.94-2.84 (m, 1H), 2.81-2.73 (m, 2H), 2.62-2.56 (m, 1H), 2.47-2.38 (m, 1H), 2.23-2.14 (m, 2H), 2.11-1.99 (m, 3H), 1.97-1.86 (m, 3H), 1.84-1.69 (m, 3H), 1.63 (s, 6H), 1.47-1.35 (m, 1H), 1.29-1.19 (m, 1H), 1.16-0.94 (m, 3H). [M+H]+=819.6.

### Example 307: 3-cyano-N-(2-((1S,4r)-4-((S)-4-(1-(4-((R)-2,6-dioxopiperidin-3-yl)-3,5-difluorophenyl)piperidin-4-yl)-2-methylpiperazin-1-yl)cyclohexyl)-6-(2-hydroxypropan-2-yl)-2H-indazol-5-yl)pyrrolo[1,2-b]pyridazine-7-carboxamide

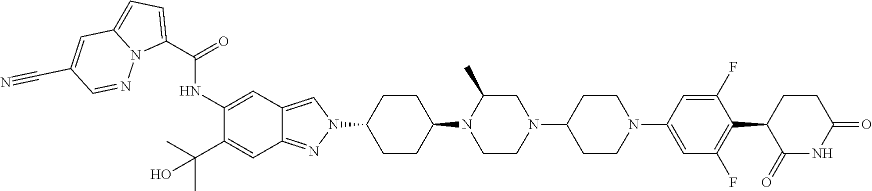 [SMILES: C[C@H]1CN(C2CCN(c3cc(F)c([C@H]4CCC(=O)NC4=O)c(F)c3)CC2)CCN1[C@H]1CC[C@H](n2cc3cc(NC(=O)c4ccc5cc(C#N)cnn45)c(C(C)(C)O)cc3n2)CC1]

The title compound (6 mg, 7%) was prepared in a manner similar to that described in Example 305. ¹H NMR (500 MHz, DMSO) δ 12.02 (s, 1H), 10.87 (s, 1H), 8.93 (d, J=2.1 Hz, 1H), 8.74 (d, J=2.1 Hz, 1H), 8.55 (s, 1H), 8.33 (s, 1H), 8.20 (s, OH), 7.72 (d, J=5.0 Hz, 1H), 7.57 (s, 1H), 7.09 (d, J=4.5 Hz, 1H), 6.62 (d, J=12.5 Hz, 2H), 5.72 (s, 1H), 4.46-4.37 (m, 1H), 4.04 (dd, J=12.5, 5.0 Hz, 1H), 3.77 (d, J=12.5 Hz, 2H), 2.92-2.85 (m, 1H), 2.81-2.63 (m, 7H), 2.55-2.52 (m, 1H), 2.40-2.35 (m, 1H), 2.34-2.28 (m, 1H), 2.26-2.20 (m, 1H), 2.19-2.12 (m, 2H), 2.10-1.85 (m, 6H), 1.83-1.77 (m, 2H), 1.74-1.67 (m, 2H), 1.63 (s, 6H), 1.48-1.34 (m, 3H), 1.02 (d, J=6.0 Hz, 3H). [M+H]⁺=847.6.

### Example 149: (R)-3-cyano-N-(2-(4-(4-(1-(4-(2,6-dioxopiperidin-3-yl)-3,5-difluorophenyl)azetidine-3-carbonyl)piperazin-1-yl)bicyclo[2.2.2]octan-1-yl)-6-(2-hydroxypropan-2-yl)-2H-indazol-5-yl)pyrrolo[1,2-b]pyridazine-7-carboxamide

### Step 1: tert-butyl (4-(4-benzylpiperazin-1-llbicyclo[2.2.2]octan-1-yl)carbamate

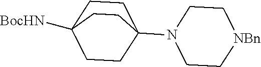 [SMILES: CC(C)(C)OC(=O)NC12CCC(N3CCN(Cc4ccccc4)CC3)(CC1)CC2]

To a solution of tert-butyl (4-aminobicyclo[2.2.2]octan-1-yl)carbamate (2.4 g, 10 mmol) and N-benzyl-2-chloro-N-(2-chloroethyl)ethan-1-amine hydrochloride (3.2 g, 12 mmol) in EtOH (80 mL) was added Na₂CO₃ (3.7 g, 35 mmol). The mixture was stirred at 80° C. for 16 h. The solvent was removed by reduced pressure. The residue was washed water (100 mL), extracted with dichloromethane (3×150 mL). The organic phase was dried over anhydrous sodium sulfate, filtered and concentrated under reduced pressure to afford the crude product, which was purified with silica gel column chromatography (DCM:MeOH=100:0˜30:1 gradient elution) to give the product (1.5 g, 38%). [M+H]⁺=400.6.

### Step 2: 4-(4-benzylpiperazin-1-yl)bicyclo[2.2.2]octan-1-amine

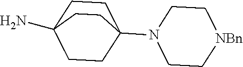 [SMILES: NC12CCC(N3CCN(Cc4ccccc4)CC3)(CC1)CC2]

To a solution of tert-butyl (4-(4-benzylpiperazin-1-yl)bicyclo[2.2.2]octan-1-yl)carbamate (1.5 g, 3.76 mmol) in DCM (20 mL) was dropwise HCl/Dioxane (9.4 mL, 4N, 37.6 mmol). The reaction was stirred at rt for 4 h. The mixture was then cooled. The mixture was concentrated under reduced pressure. The residue was dissolved in water. The mixture pH value was adjusted to 10-11 by saturated Na₂CO₃. The mixture was extracted with DCM (3×60 mL). The organic phase was dried over anhydrous sodium sulfate, filtered and concentrated under reduced pressure to afford the crude product (1.1 g, 98%). [M+H]⁺=300.4.

### Step 3: methyl 2-(4-(4-benzylpiperazin-1-yl)bicyclo[2.2.2]octan-1-yl)-2H-indazole-6-carboxylate

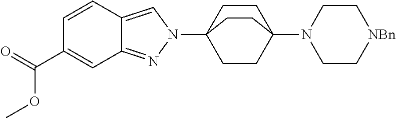 [SMILES: COC(=O)c1ccc2cn(C34CCC(N5CCN(Cc6ccccc6)CC5)(CC3)CC4)nc2c1]

A solution of 4-(4-benzylpiperazin-1-yl)bicyclo[2.2.2]octan-1-amine (1.1 g, 3.68 mmol) and methyl 4-formyl-3-nitrobenzoate (769 mg, 3.68 mmol) in IPA (15 mL) was stirred at 85° C. for 4 h. Then, the reaction was cooled down to room temperature. Bu₃P (2.23 g, 11.04 mmol) was added to the reaction. The reaction was stirred at 85° C. for 12 h. The solvent was removed by reduced pressure. The residue was slurry with PE (3×10 mL) and filtered to afford the crude product, which was purified with silica gel column chromatography (DCM:MeOH=100:0˜19:1 gradient elution) to give the product (1.17 g, 69%). [M+H]⁺=459.4

### Step 4: methyl 2-(4-(piperazin-1-yl)bicyclo[2.2.2]octan-1-yl)-2H-indazole-6-carboxylate hydrochloride

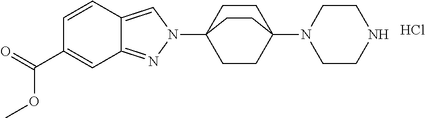 [SMILES: COC(=O)c1ccc2cn(C34CCC(N5CCNCC5)(CC3)CC4)nc2c1 ; Cl]

To a solution of methyl 2-(4-(4-benzylpiperazin-1-yl)bicyclo[2.2.2]octan-1-yl)-2H-indazole-6-carboxylate (1.17 g, 2.55 mmol) in ACN (25 mL) was added 1-chloroethyl carbonochloridate (1.81 g, 12.76 mmol). The mixture was stirred at 80° C. for 16 h. The solvent was removed by reduced pressure. The residue was dissolved in MeOH (30 mL). The mixture was stirred at 50° C. for 3 h. The mixture was concentrated in vacuum to afford the product (950 mg, 92%). [M+H]⁺=369.3.

### Step 5: methyl 2-(4-(4-(tert-butoxycarbonyl)piperazin-1-yl)bicyclo[2.2.2]octan-1-yl)-5-nitro-2H-indazole-6-carboxylate

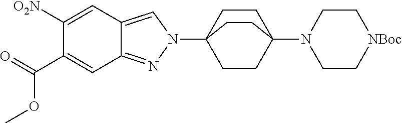 [SMILES: COC(=O)c1cc2nn(C34CCC(N5CCN(C(=O)OC(C)(C)C)CC5)(CC3)CC4)cc2cc1[N+](=O)[O-]]

To a solution of methyl 2-(4-(piperazin-1-yl)bicyclo[2.2.2]octan-1-yl)-2H-indazole-6-carboxylate hydrochloride (950 mg, 2.35 mmol) in H₂SO₄ (8 mL) was dropwise HNO₃ (342 mg, 65%, 3.53 mmol) at 0° C. The mixture was stirred at room temperature for 16 h. The mixture pH value was adjusted to 10-11 by saturated Na₂CO₃. Na₂CO₃ (374 mg, 3.53 mmol) in water and THF (60 mL) were dropwise to mixture. Then, (Boc)₂O (615 mg, 2.82 mmol) was added to the reaction. The mixture was stirred at room temperature for 4 h. The mixture was washed by water (100 mL), extracted with dichloromethane (3×100 mL). The organic phase was dried over anhydrous sodium sulfate, filtered and concentrated under reduced pressure to afford the crude product, which was purified with silica gel column chromatography (DCM:MeOH=100:0˜19:1 gradient elution) to give the product (1.05 g, 87%). [M+H]⁺=514.6.

### Step 6: methyl 5-amino-2-(4-(4-(tert-butoxycarbonyl)piperazin-1-yl)bicyclo[2.2.2]octan-1-yl)-2H-indazole-6-carboxylate

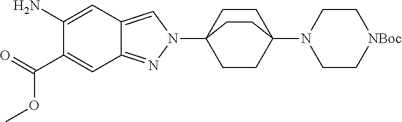 [SMILES: COC(=O)c1cc2nn(C34CCC(N5CCN(C(=O)OC(C)(C)C)CC5)(CC3)CC4)cc2cc1N]

To a solution of methyl 2-(4-(4-(tert-butoxycarbonyl)piperazin-1-yl)bicyclo[2.2.2]octan-1-yl)-5-nitro-2H-indazole-6-carboxylate (1.05 g, 2.05 mmol) and Fe(574 mg, 10.25 mmol) in EtOH (20 mL) was dropwise NH₄Cl (548 mg, 10.25 mmol) in water. The mixture was stirred 85° C. for 4 h. The mixture pH value was adjusted to 10-11 by saturated Na₂CO₃. The mixture was filtered by celite. The filtrate was concentrated under reduced pressure to afford the crude product, which was purified with silica gel column chromatography (DCM:MeOH=100:0˜19:1 gradient elution) to give the product (460 mg, 46%). [M+H]⁺=484.5.

### Step 7: methyl 2-(4-(4-(tert-butoxycarbonyl)piperazin-1-yl)bicyclo[2.2.2]octan-1-yl)-5-(2,2,2-trifluoroacetamido)-2H-indazole-6-carboxylate

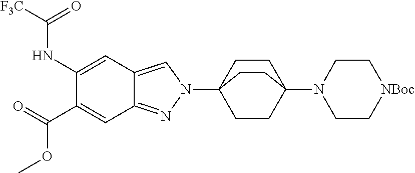 [SMILES: COC(=O)c1cc2nn(C34CCC(N5CCN(C(=O)OC(C)(C)C)CC5)(CC3)CC4)cc2cc1NC(=O)C(F)(F)F]

To a solution of methyl 5-amino-2-(4-(4-(tert-butoxycarbonyl)piperazin-1-yl)bicyclo[2.2.2]octan-1-yl)-2H-indazole-6-carboxylate (460 mg, 0.95 mmol) in DCM (6 mL) was dropwise TFAA (240 mg, 1.14 mmol). The mixture was stirred room temperature for 1 h. The mixture pH value was adjusted to 10-11 by saturated Na₂CO₃. The mixture was washed by water (20 mL) and extracted with DCM (3×20 mL). The mixture was dried over Na₂SO₄ and filtered. The filtrate was concentrated under reduced pressure to afford the crude product. The crude product was slurry by PE (3×6 mL) to give the product (380 mg, 69%). [M+H]⁺=580.6.

### Step 8: tert-butyl 4-(4-(6-(2-hydroxypropan-2-yl)-5-(2,2,2-trifluoroacetamido)-2H-indazol-2-yl)bicyclo[2.2.2]octan-1-yl)piperazine-1-carboxylate

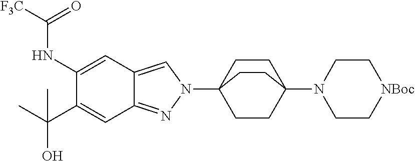 [SMILES: CC(C)(C)OC(=O)N1CCN(C23CCC(n4cc5cc(NC(=O)C(F)(F)F)c(C(C)(C)O)cc5n4)(CC2)CC3)CC1]

To a solution of methyl 2-(4-(4-(tert-butoxycarbonyl)piperazin-1-yl)bicyclo[2.2.2]octan-1-yl)-5-(2,2,2-trifluoroacetamido)-2H-indazole-6-carboxylate (380 mg, 0.66 mmol) and LiCl (139 mg, 3.28 mmol) in THF (10 mL) was dropwise MeMgBr (6.6 mL, 1M, 6.6 mmol) at 0° C. The mixture was stirred room temperature for 6 h. The mixture was quenched by saturated NH₄Cl at 0° C. The mixture was washed by water (20 mL) and extracted with EtOAc (3×30 mL). The mixture was dried over Na₂SO₄ and filtered. The filtrate was concentrated under reduced pressure to afford the crude product, which was purified with silica gel column chromatography (DCM:MeOH=100:0˜19:1 gradient elution) to give the product (250 mg, 66%). [M+H]⁺=580.6.

### Step 9: tert-butyl 4-(4-(5-amino-6-(2-hydroxypropan-2-yl)-2H-indazol-2-yl)bicyclo[2.2.2]octan-1-yl)piperazine-1-carboxylate

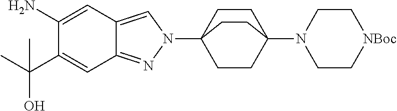 [SMILES: CC(C)(C)OC(=O)N1CCN(C23CCC(n4cc5cc(N)c(C(C)(C)O)cc5n4)(CC2)CC3)CC1]

To a solution of tert-butyl 4-(4-(6-(2-hydroxypropan-2-yl)-5-(2,2,2-trifluoroacetamido)-2H-indazol-2-yl)bicyclo[2.2.2]octan-1-yl)piperazine-1-carboxylate (250 mg, 0.43 mmol) in MeOH (6 mL) was NaOH (2.15 mL, 2N, 4.3 mmol). The mixture was stirred 70° C. for 2 h. The mixture was filtered to give the product (185 mg, 89%). [M+H]⁺=484.6.

### Step 10: tert-butyl 4-(4-(5-(3-cyanopyrrolo[1,2-b]pyridazine-7-carboxamido)-6-(2-hydroxypropan-2-yl)-2H-indazol-2-yl)bicyclo[2.2.2]octan-1-yl)piperazine-1-carboxylate

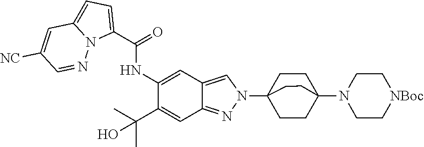 [SMILES: CC(C)(C)OC(=O)N1CCN(C23CCC(n4cc5cc(NC(=O)c6ccc7cc(C#N)cnn67)c(C(C)(C)O)cc5n4)(CC2)CC3)CC1]

To a solution of tert-butyl 4-(4-(5-amino-6-(2-hydroxypropan-2-yl)-2H-indazol-2-yl)bicyclo[2.2.2]octan-1-yl)piperazine-1-carboxylate (185 mg, 0.38 mmol), DIEA (147 mg, 1.14 mmol) and 3-cyanopyrrolo[1,2-b]pyridazine-7-carboxylic acid (71.6 mg, 0.38 mmol) in DMF (3 mL) was added HATU (159 mg, 0.42 mmol). The reaction was stirred at rt for 4 h. The solvent was removed by reduced pressure. The residue was washed water (15 mL), extracted with dichloromethane (3×20 mL). The organic layers were combined and washed with brine (3×30 mL). The organic phase was dried over anhydrous sodium sulfate, filtered and concentrated under reduced pressure to afford the crude product, which was purified with silica gel column chromatography (DCM:MeOH=100:0˜18:1 gradient elution) to give the product (215 mg, 87% yield). [M+H]⁺=653.3.

### Step 11: 3-cyano-N-(6-(2-hydroxypropan-2-yl)-2-(4-(piperazin-1-yl)bicyclo[2.2.2]octan-1-yl)-2H-indazol-5-yl)pyrrolo[1,2-b]pyridazine-7-carboxamide

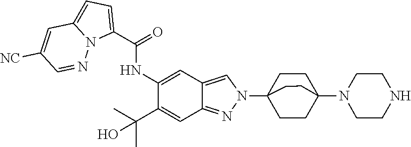 [SMILES: CC(C)(O)c1cc2nn(C34CCC(N5CCNCC5)(CC3)CC4)cc2cc1NC(=O)c1ccc2cc(C#N)cnn12]

To a solution of tert-butyl 4-(4-(5-(3-cyanopyrrolo[1,2-b]pyridazine-7-carboxamido)-6-(2-hydroxypropan-2-yl)-2H-indazol-2-yl)bicyclo[2.2.2]octan-1-yl)piperazine-1-carboxylate (215 mg, 0.33 mmol) in DCM (5 mL) was dropwise TFA (1 mL). The reaction was stirred at rt for 3 h. The mixture pH value was adjusted to 10-11 by saturated Na₂CO₃. The mixture was washed water (15 mL), extracted with dichloromethane (3×20 mL) The organic phase was dried over anhydrous sodium sulfate, filtered and concentrated under reduced pressure to afford the product (175 mg, 96% yield). [M+H]⁺=553.6.

### Step 12: (R)-3-cyano-N-(2-(4-(4-(1-(4-(2,6-dioxopiperidin-3-yl)-3,5-difluorophenyl)azetidine-3-carbonyl)piperazin-1-yl)bicyclo[2.2.2]octan-1-yl)-6-(2-hydroxypropan-2-yl)-2H-indazol-5-yl)pyrrolo[1,2-b]pyridazine-7-carboxamide

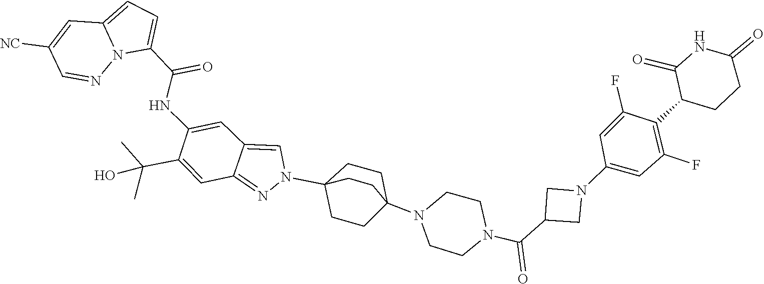 [SMILES: CC(C)(O)c1cc2nn(C34CCC(N5CCN(C(=O)C6CN(c7cc(F)c([C@H]8CCC(=O)NC8=O)c(F)c7)C6)CC5)(CC3)CC4)cc2cc1NC(=O)c1ccc2cc(C#N)cnn12]

To a solution of 3-cyano-N-(6-(2-hydroxypropan-2-yl)-2-(4-(piperazin-1-yl)bicyclo[2.2.2]octan-1-yl)-2H-indazol-5-yl)pyrrolo[1,2-b]pyridazine-7-carboxamide (55.2 mg, 0.1 mmol), DIEA (40 mg, 0.3 mmol) and (R)-1-(4-(2,6-dioxopiperidin-3-yl)-3,5-difluorophenyl)azetidine-3-carboxylic acid (32.4 mg, 0.1 mmol) in DMF (5 mL) was added T₃P (125.2 mg, 50%, 0.2 mmol). The reaction was stirred at rt for 4 h. The mixture was washed water (15 mL), extracted with dichloromethane (3×20 mL). The organic layers were combined and washed with brine (3×30 mL). The organic phase was dried over anhydrous sodium sulfate, filtered and concentrated under reduced pressure to afford the crude product, which was purified with silica gel column chromatography (DCM:MeOH=100:0˜18:1 gradient elution) to give the product (15 mg, 17% yield). [M+H]⁺=859.7. ¹H NMR (500 MHz, DMSO) δ 12.02 (s, 1H), 10.86 (s, 1H), 8.93 (d, J=2.1 Hz, 1H), 8.74 (d, J=2.0 Hz, 1H), 8.55 (s, 1H), 8.35 (s, 1H), 7.71 (s, 1H), 7.57 (s, 1H), 7.09 (d, J=4.7 Hz, 1H), 6.17 (d, J=11.1 Hz, 2H), 5.71 (s, 1H), 4.09-4.01 (m, 3H), 3.96-3.76 (m, 3H), 3.49-3.41 (m, 2H), 3.28 (dd, J=6.1, 4.0 Hz, 2H), 2.84-2.73 (m, 1H), 2.60-2.52 (m, 5H), 2.27-2.16 (m, 6H), 2.12-2.03 (m, 1H), 1.98-1.90 (m, 1H), 1.84-1.75 (m, 6H), 1.63 (s, 6H).

### Example 151: (R)-3-cyano-N-(2-(4-(4-(4-(2,6-dioxopiperidin-3-yl)-3,5-difluorophenethyl)piperazin-1-yl)bicyclo[2.2.2]octan-1-yl)-6-(2-hydroxypropan-2-yl)-2H-indazol-5-yl)pyrrolo[1,2-b]pyridazine-7-carboxamide

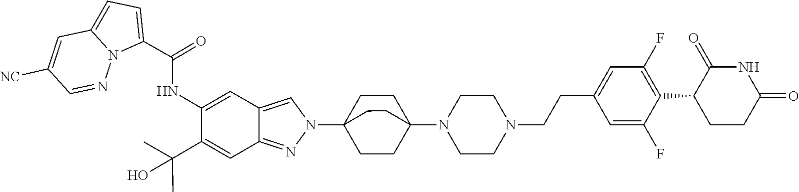 [SMILES: CC(C)(O)c1cc2nn(C34CCC(N5CCN(CCc6cc(F)c([C@H]7CCC(=O)NC7=O)c(F)c6)CC5)(CC3)CC4)cc2cc1NC(=O)c1ccc2cc(C#N)cnn12]

To a solution of 3-cyano-N-(6-(2-hydroxypropan-2-yl)-2-(4-(piperazin-1-yl)bicyclo[2.2.2]octan-1-yl)-2H-indazol-5-yl)pyrrolo[1,2-b]pyridazine-7-carboxamide (55.2 mg, 0.1 mmol) and (R)-2-(4-(2,6-dioxopiperidin-3-yl)-3,5-difluorophenyl)acetaldehyde (26.7 mg, 0.1 mmol) in DCE (8 mL) was added NaBH(OAc)₃ (53 mg, 0.25 mmol). The mixture was stirred at 50° C. for 4 h. The solvent was removed by reduced pressure. The residue was washed water (20 mL), extracted with dichloromethane (3×30 mL). The organic phase was dried over anhydrous sodium sulfate, filtered and concentrated under reduced pressure to afford the crude product, which was purified with silica gel column chromatography (DCM:MeOH=100:0˜9:1 gradient elution) to give the product (18 mg, 22%). [M+H]⁺=804.7.

¹H NMR (500 MHz, DMSO) δ 12.02 (s, 1H), 10.94 (s, 1H), 8.93 (d, J=2.1 Hz, 1H), 8.74 (d, J=2.1 Hz, 1H), 8.54 (s, 1H), 8.35 (s, 1H), 7.71 (d, J=4.8 Hz, 1H), 7.57 (s, 1H), 7.09 (d, J=4.8 Hz, 1H), 7.02 (d, J=10.1 Hz, 2H), 5.71 (s, 1H), 4.19 (dt, J=11.4, 5.8 Hz, 1H), 2.85-2.71 (m, 3H), 2.61-2.51 (m, 7H), 2.47-2.38 (m, 3H), 2.28-2.06 (m, 8H), 2.04-1.96 (m, 1H), 1.81-1.73 (m, 6H), 1.63 (s, 6H).

### Example 207: N-(2-(trans)-4-(4-(1-(4-((R)-2,6-dioxopiperidin-3-yl)-3,5-difluorophenyl)azetidine-3-carbonyl)piperazin-1-yl)cyclohexyl)-6-(2-hydroxypropan-2-yl)-2H-indazol-5-yl)-3-(trifluoromethyl)pyrrolo[1,2-b]pyridazine-7-carboxamide

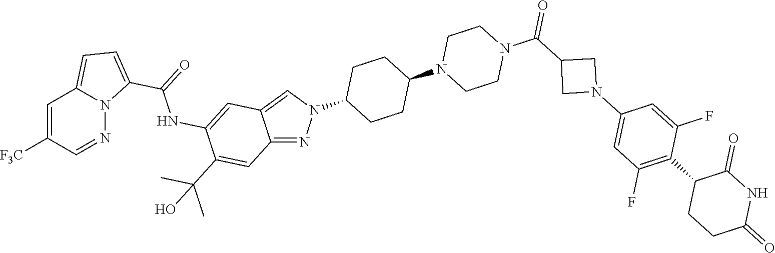 [SMILES: CC(C)(O)c1cc2nn([C@H]3CC[C@H](N4CCN(C(=O)C5CN(c6cc(F)c([C@H]7CCC(=O)NC7=O)c(F)c6)C5)CC4)CC3)cc2cc1NC(=O)c1ccc2cc(C(F)(F)F)cnn12]

The title compound (10 mg, 14% yield) was prepared in a manner similar to that described in Example 178. ¹H NMR (500 MHz, DMSO) δ 12.08 (s, 1H), 10.86 (s, 1H), 8.86-8.76 (m, 2H), 8.58 (s, 1H), 8.34 (s, 1H), 7.73 (d, J=4.7 Hz, 1H), 7.57 (s, 1H), 7.06 (d, J=4.7 Hz, 1H), 6.17 (d, J=11.0 Hz, 2H), 5.71 (s, 1H), 4.52-4.39 (m, 1H), 4.08-3.98 (m, 3H), 3.95-3.79 (m, 3H), 3.55-3.43 (m, 2H), 3.30-3.27 (m, OH), 2.78 (tt, J=13.6, 4.9 Hz, 1H), 2.66-2.51 (m, 3H), 2.46 (d, J=6.4 Hz, 1H), 2.23-2.14 (m, 2H), 2.13-1.90 (m, 6H), 1.65 (s, 6H), 1.57-1.42 (m, 2H). [M+H]⁺=876.7.

### Example 259: 3-cyano-N-(2-(trans)-4-(4-(1-(4-((R)-2,6-dioxopiperidin-3-yl)-3-methoxyphenyl)azetidine-3-carbonyl)piperazin-1-yl)cyclohexyl)-6-(2-hydroxypropan-2-yl)-2H-indazol-5-yl)pyrrolo[1,2-b]pyridazine-7-carboxamide

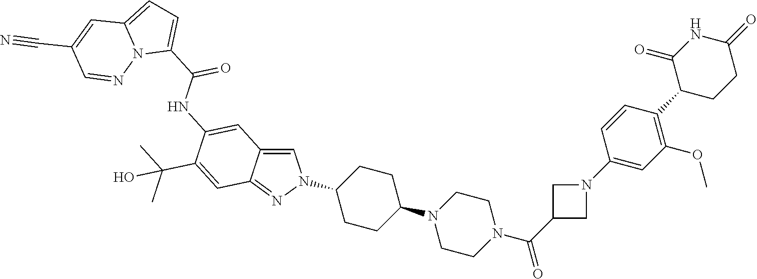 [SMILES: COc1cc(N2CC(C(=O)N3CCN([C@H]4CC[C@H](n5cc6cc(NC(=O)c7ccc8cc(C#N)cnn78)c(C(C)(C)O)cc6n5)CC4)CC3)C2)ccc1[C@H]1CCC(=O)NC1=O]

The title compound (16 mg, 19%) was prepared in a manner similar to that described in Example 178. ¹H NMR (500 MHz, DMSO) δ 12.02 (s, 1H), 10.65 (s, 1H), 8.93 (d, J=2.2 Hz, 1H), 8.74 (d, J=2.2 Hz, 1H), 8.55 (s, 1H), 8.34 (s, 1H), 7.72 (d, J=4.8 Hz, 1H), 7.57 (s, 1H), 7.09 (d, J=4.8 Hz, 1H), 6.89 (d, J=8.2 Hz, 1H), 6.07 (d, J=2.0 Hz, 1H), 5.99 (dd, J=8.1, 2.0 Hz, 1H), 5.72 (s, 1H), 4.49-4.38 (m, 1H), 4.06-3.97 (m, 2H), 3.91-3.85 (m, 2H), 3.84-3.74 (m, 2H), 3.71 (s, 3H), 3.52-3.45 (m, 2H), 3.33 (s, 2H), 2.63 (ddd, J=17.3, 12.3, 5.2 Hz, 1H), 2.56 (dd, J=14.7, 1.7 Hz, 4H), 2.47-2.41 (m, 2H), 2.22-2.08 (m, 3H), 2.01-1.90 (m, 4H), 1.87-1.82 (m, 1H), 1.63 (s, 6H), 1.57-1.44 (m, 2H). [M+H]⁺=827.9.

### Example 269: 3-cyano-N-(2-(trans)-4-(4-(1-(4-(2,4-dioxotetrahydropyrimidin-1(2H)-yl)-3-methoxyphenyl)azetidine-3-carbonyl)piperazin-1-yl)cyclohexyl)-6-(2-hydroxypropan-2-yl)-2H-indazol-5-yl)pyrrolo[1,2-b]pyridazine-7-carboxamide

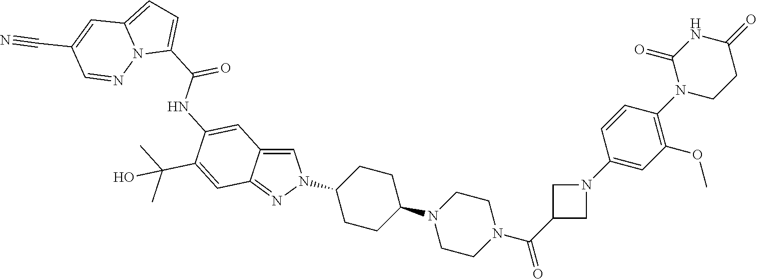 [SMILES: COc1cc(N2CC(C(=O)N3CCN([C@H]4CC[C@H](n5cc6cc(NC(=O)c7ccc8cc(C#N)cnn78)c(C(C)(C)O)cc6n5)CC4)CC3)C2)ccc1N1CCC(=O)NC1=O]

The title compound (13 mg, 15%) was prepared in a manner similar to that described in Example 178. ¹H NMR (500 MHz, DMSO) δ 12.02 (s, 1H), 10.18 (s, 1H), 8.94 (d, J=2.2 Hz, 1H), 8.74 (d, J=2.2 Hz, 1H), 8.55 (s, 1H), 8.34 (s, 1H), 7.72 (d, J=4.8 Hz, 1H), 7.57 (s, 1H), 7.09 (d, J=4.8 Hz, 1H), 7.00 (d, J=8.3 Hz, 1H), 6.11 (d, J=2.3 Hz, 1H), 6.01 (dd, J=8.4, 2.3 Hz, 1H), 5.76 (d, J=40.1 Hz, 1H), 4.51-4.39 (m, 1H), 4.05 (dd, J=14.4, 6.6 Hz, 2H), 3.93 (dd, J=19.8, 7.5 Hz, 2H), 3.89-3.83 (m, 1H), 3.76 (s, 3H), 3.58-3.46 (m, 4H), 3.36 (dd, J=7.4, 3.6 Hz, 2H), 2.69-2.52 (m, 5H), 2.23-2.17 (m, 2H), 2.07-1.86 (m, 4H), 1.63 (s, 6H), 1.55-1.46 (m, 2H). [M+H]⁺=829.0.

### Example 291: 3-cyano-N-(2-(trans)-4-(4-(1-(4-((R)-2,6-dioxopiperidin-3-yl)-3,5-difluorophenyl)piperidine-4-carbonyl)piperazin-1-yl)cyclohexyl)-6-(2-hydroxypropan-2-yl)-2H-indazol-5-yl)pyrrolo[1,2-b]pyridazine-7-carboxamide

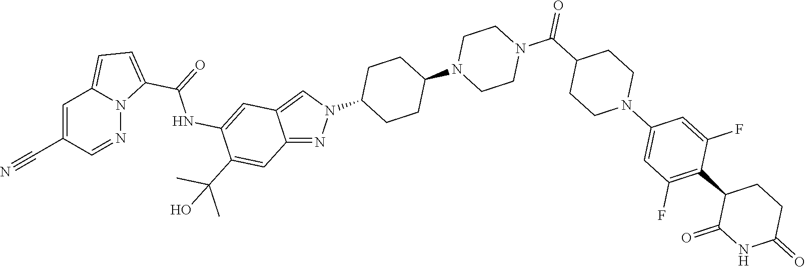 [SMILES: CC(C)(O)c1cc2nn([C@H]3CC[C@H](N4CCN(C(=O)C5CCN(c6cc(F)c([C@H]7CCC(=O)NC7=O)c(F)c6)CC5)CC4)CC3)cc2cc1NC(=O)c1ccc2cc(C#N)cnn12]

The title compound (25 mg, 21%) was prepared in a manner similar to that described in Example 178. ¹H NMR (500 MHz, DMSO) δ 12.02 (s, 1H), 10.87 (s, 1H), 8.94 (d, J=2.1 Hz, 1H), 8.74 (d, J=2.1 Hz, 1H), 8.55 (s, 1H), 8.34 (s, 1H), 7.72 (d, J=4.8 Hz, 1H), 7.57 (s, 1H), 7.09 (d, J=4.8 Hz, 1H), 6.63 (d, J=12.8 Hz, 2H), 5.72 (s, 1H), 4.48-4.36 (m, 1H), 4.09-3.99 (m, 1H), 3.84-3.72 (m, 2H), 3.61-3.41 (m, 4H), 3.30-3.27 (m, 1H), 2.93-2.73 (m, 4H), 2.59-2.52 (m, 3H), 2.24-2.02 (m, 4H), 2.01-1.89 (m, 5H), 1.71-1.41 (m, 13H). [M+H]⁺=861.8.

### Example 319: 3-cyano-N-(2-(trans)-4-((S)-4-(1-(4-((R)-2,6-dioxopiperidin-3-yl)-3,5-difluorophenyl)piperidin-4-yl)-3-(methoxymethyl)piperazin-1-yl)cyclohexyl)-6-(2-hydroxypropan-2-yl)-2H-indazol-5-yl)pyrrolo[1,2-b]pyridazine-7-carboxamide

### Step 1: methyl 2-(trans)-4-((S)-4-(tert-butoxycarbonyl)-3-(methoxymethyl)piperazin-1-yl)cyclohexyl)-2H-indazole-6-carboxylate

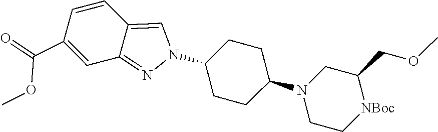

A solution of tert-butyl (S)-4-(trans)-4-aminocyclohexyl)-2-(methoxymethyl)piperazine-1-carboxylate (7.5 g, 22.9 mmol) and methyl 4-formyl-3-nitrobenzoate (4.8 g, 22.9 mmol) in IPA (130 mL) was stirred at 85° C. for 4 h. Then, the reaction was cooled down to room temperature. Bu₃P (13.88 g, 68.7 mmol) was dropwise to the reaction. The reaction was stirred at 85° C. for 12 h. The solvent was removed by reduced pressure. The residue was slurry with PE (3×50 mL) and filtered to afford the product (4.87 g, 44%). [M+H]⁺=487.5.

### Step 2: methyl 2-(trans)-4-((S)-4-(tert-butoxycarbonyl)-3-(methoxymethyl)piperazin-1-yl)cyclohexyl)-5-nitro-2H-indazole-6-carboxylate

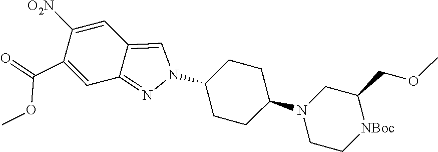

To a solution of methyl 2-(trans)-4-((S)-4-(tert-butoxycarbonyl)-3-(methoxymethyl) piperazin-1-yl)cyclohexyl)-2H-indazole-6-carboxylate (4.87 g, 10 mmol) in H₂SO₄ (50 mL) was dropwise HNO₃ (1.45 g, 65%, 15 mmol) at 0° C. The mixture was stirred at room temperature for 16 h. The mixture pH value was adjusted to 10-11 by saturated Na₂CO₃. Na₂CO₃ (1.59 g, 15 mmol) in water and THF (160 mL) were dropwise to mixture. Then, (Boc)₂O (2.62 g, 12 mmol) was added to the reaction. The mixture was stirred at room temperature for 4 h. The mixture was washed by water (200 mL), extracted with dichloromethane (3×200 mL). The organic phase was dried over anhydrous sodium sulfate, filtered and concentrated under reduced pressure to afford the crude product, which was purified with silica gel column chromatography (DCM:MeOH=100:0˜19:1 gradient elution) to give the product (3.5 g, 66%). [M+H]⁺=532.6.

### Step 3: methyl 5-amino-2-(trans)-4-((S)-4-(tert-butoxycarbonyl)-3-(methoxymethyl) piperazin-1-yl)cyclohexyl)-2H-indazole-6-carboxylate

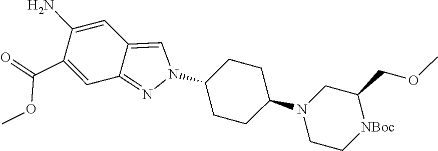

To a solution of methyl 2-(trans)-4-((S)-4-(tert-butoxycarbonyl)-3-(methoxymethyl)piperazin-1-yl)cyclohexyl)-5-nitro-2H-indazole-6-carboxylate (3.5 g, 6.58 mmol) and Fe (1.84 g, 32.9 mmol) in EtOH (50 mL) was dropwise NH₄Cl (1.76 g, 32.9 mmol) in water. The mixture was stirred 85° C. for 4 h. The mixture pH value was adjusted to 10-11 by saturated Na₂CO₃. The mixture was filtered by celite. The filtrate was concentrated under reduced pressure to afford the crude product, which was purified with silica gel column chromatography (DCM:MeOH=100:0˜19:1 gradient elution) to give the product (1.5 g, 45%). [M+H]⁺=502.5.

### Step 4: methyl 2-(trans)-4-((S)-4-(tert-butoxycarbonyl)-3-(methoxymethyl)piperazin-1-yl)cyclohexyl)-5-(2,2,2-trifluoroacetamido)-2H-indazole-6-carboxylate

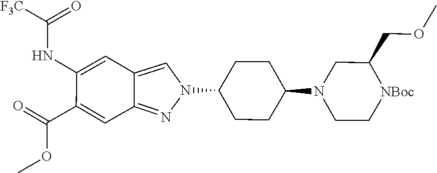

To a solution of methyl 5-amino-2-(trans)-4-((S)-4-(tert-butoxycarbonyl)-3-(methoxymethyl)piperazin-1-yl)cyclohexyl)-2H-indazole-6-carboxylate (1.5 g, 2.99 mmol) in DCM (20 mL) was added dropwise 2,2,2-trifluoroacetic anhydride (240 mg, 4.49 mmol). The mixture was stirred room temperature for 1 h. The mixture pH value was adjusted to 10-11 by saturated Na₂CO₃. The mixture was washed by water (20 mL) and extracted with DCM (3×30 mL). The mixture was dried over Na₂SO₄ and filtered. The filtrate was concentrated under reduced pressure to afford the crude product. The crude product was slurry by PE (3×6 mL) to give the product (1.25 g, 70%). [M+H]⁺=598.6.

### Step 5: tert-butyl (S)-4-(trans)-4-(6-(2-hydroxypropan-2-yl)-5-(2,2,2-trifluoroacetamido)-2H-indazol-2-yl)cyclohexyl)-2-(methoxymethyl)piperazine-1-carboxylate

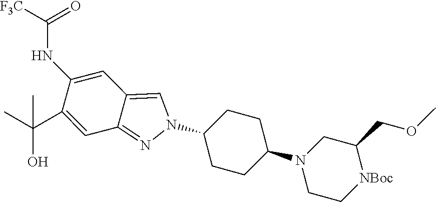 [SMILES: COC[C@@H]1CN([C@H]2CC[C@H](n3cc4cc(NC(=O)C(F)(F)F)c(C(C)(C)O)cc4n3)CC2)CCN1C(=O)OC(C)(C)C]

To a solution of methyl 2-(trans)-4-((S)-4-(tert-butoxycarbonyl)-3-(methoxymethyl)piperazin-1-yl)cyclohexyl)-5-(2,2,2-trifluoroacetamido)-2H-indazole-6-carboxylate (1.25 g, 2.09 mmol) and LiCl (444 mg, 10.45 mmol) in THF (10 mL) was dropwise CH₃MgBr (20.9 mL, 1M, 20.9 mmol) at 0° C. The mixture was stirred room temperature for 6 h. The mixture was quenched by saturated NH₄Cl at 0° C. The mixture was washed by water (30 mL) and extracted with EtOAc (3×50 mL). The mixture was dried over Na₂SO₄ and filtered. The filtrate was concentrated under reduced pressure to afford the crude product, which was purified with silica gel column chromatography (DCM:MeOH=100:0˜19:1 gradient elution) to give the product (750 mg, 60%). [M+H]⁺=598.6

### Step 9: tert-butyl (S)-4-(trans)-4-(5-amino-6-(2-hydroxypropan-2-yl)-2H-indazol-2-yl)cyclohexyl)-2-(methoxymethyl)piperazine-1-carboxylate

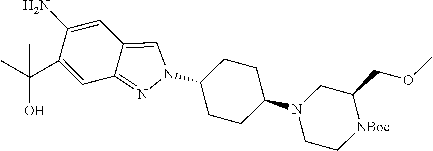 [SMILES: COC[C@@H]1CN([C@H]2CC[C@H](n3cc4cc(N)c(C(C)(C)O)cc4n3)CC2)CCN1C(=O)OC(C)(C)C]

To a solution of tert-butyl (S)-4-(trans)-4-(6-(2-hydroxypropan-2-yl)-5-(2,2,2-trifluoroacetamido)-2H-indazol-2-yl)cyclohexyl)-2-(methoxymethyl)piperazine-1-carboxylate (750 mg, 1.26 mmol) in MeOH (16 mL) was NaOH (6.3 mL, 2N, 12.6 mmol). The mixture was stirred 70° C. for 2 h. The mixture was filtered to give the product (450 mg, 73%). [M+H]⁺=502.6

### Step 6: tert-butyl (S)-4-(trans)-4-(5-(3-cyanopyrrolo[1,2-b]pyridazine-7-carboxamido)-6-(2-hydroxypropan-2-yl)-2H-indazol-2-yl)cyclohexyl)-2-(methoxymethyl)piperazine-1-carboxylate

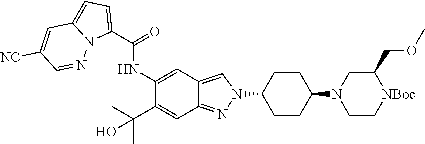 [SMILES: COC[C@@H]1CN([C@H]2CC[C@H](n3cc4cc(NC(=O)c5ccc6cc(C#N)cnn56)c(C(C)(C)O)cc4n3)CC2)CCN1C(=O)OC(C)(C)C]

To a solution of tert-butyl (S)-4-(trans)-4-(5-amino-6-(2-hydroxypropan-2-yl)-2H-indazol-2-yl)cyclohexyl)-2-(methoxymethyl)piperazine-1-carboxylate (450 mg, 0.9 mmol), DIEA (348.3 mg, 2.7 mmol) and 3-cyanopyrrolo[1,2-b]pyridazine-7-carboxylic acid (168 mg, 0.9 mmol) in DMF (13 mL) was added HATU (376 mg, 0.99 mmol). The reaction was stirred at rt for 4 h. The solvent was removed by reduced pressure. The residue was washed water (45 mL), extracted with dichloromethane (3×50 mL), and washed with brine (3×100 mL). The organic phase was dried over anhydrous sodium sulfate, filtered and concentrated under reduced pressure to afford the crude product, which was purified with silica gel column chromatography (DCM:MeOH=100:0˜18:1 gradient elution) to give the product (500 mg, 83% yield). [M+H]⁺=671.3

### Step 7: 3-cyano-N-(6-(2-hydroxypropan-2-yl)-2-(trans)-4-((S)-3-(methoxymethyl)piperazin-1-yl)cyclohexyl)-2H-indazol-5-yl)pyrrolo[1,2-b]pyridazine-7-carboxamide

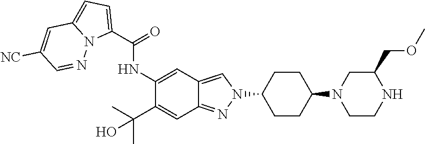 [SMILES: COC[C@@H]1CN([C@H]2CC[C@H](n3cc4cc(NC(=O)c5ccc6cc(C#N)cnn56)c(C(C)(C)O)cc4n3)CC2)CCN1]

To a solution of tert-butyl (S)-4-(trans)-4-(5-(3-cyanopyrrolo[1,2-b]pyridazine-7-carboxamido)-6-(2-hydroxypropan-2-yl)-2H-indazol-2-yl)cyclohexyl)-2-(methoxymethyl)piperazine-1-carboxylate (500 mg, 0.75 mmol) in DCM (10 mL) was dropwise TFA (3 mL). The reaction was stirred at rt for 3 h. The mixture pH value was adjusted to 10-11 by saturated Na₂CO₃. The mixture was washed water (15 mL), extracted with dichloromethane (3×20 mL) The organic phase was dried over anhydrous sodium sulfate, filtered and concentrated under reduced pressure to afford the product (418 mg, 98% yield). [M+H]⁺=571.5

### Step 8: 3-cyano-N-(2-(trans)-4-((S)-4-(1-(4-((R)-2,6-dioxopiperidin-3-yl)-3,5-difluorophenyl)piperidin-4-yl)-3-(methoxymethyl)piperazin-1-yl)cyclohexyl)-6-(2-hydroxypropan-2-yl)-2H-indazol-5-yl)pyrrolo[12-b]pyridazine-7-carboxamide

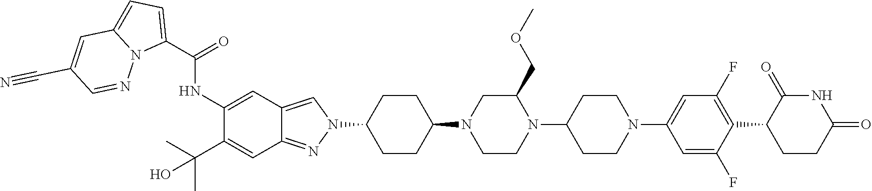 [SMILES: COC[C@@H]1CN([C@H]2CC[C@H](n3cc4cc(NC(=O)c5ccc6cc(C#N)cnn56)c(C(C)(C)O)cc4n3)CC2)CCN1C1CCN(c2cc(F)c([C@H]3CCC(=O)NC3=O)c(F)c2)CC1]

To a solution of 3-cyano-N-(6-(2-hydroxypropan-2-yl)-2-(trans)-4-((S)-3-(methoxymethyl)piperazin-1-yl)cyclohexyl)-2H-indazol-5-yl)pyrrolo[1,2-b]pyridazine-7-carboxamide (57 mg, 0.1 mmol) and (R)-3-(2,6-difluoro-4-(4-oxopiperidin-1-yl)phenyl)piperidine-2,6-dione (32.2 mg, 0.1 mmol) in DCE (8 mL) was added NaBH(OAc)₃ (53 mg, 0.25 mmol). The mixture was stirred at 50° C. for 4 h. The solvent was removed by reduced pressure. The residue was washed water (20 mL), extracted with dichloromethane (3×30 mL). The organic phase was dried over anhydrous sodium sulfate, filtered and concentrated under reduced pressure to afford the crude product, which was purified with silica gel column chromatography (DCM:MeOH=100:0˜9:1 gradient elution) to give the product (6 mg, 7%). [M+H]⁺=877.8.

¹H NMR (500 MHz, DMSO) δ 12.02 (s, 1H), 10.87 (s, 1H), 8.93 (d, J=2.1 Hz, 1H), 8.74 (d, J=2.1 Hz, 1H), 8.55 (s, 1H), 8.32 (s, 1H), 7.72 (d, J=4.8 Hz, 1H), 7.57 (s, 1H), 7.09 (d, J=4.8 Hz, 1H), 6.62 (d, J=12.8 Hz, 2H), 5.72 (s, 1H), 4.47-4.37 (m, 1H), 4.10-3.99 (m, 1H), 3.84-3.74 (m, 2H), 3.55-3.49 (m, 1H), 3.40 (dt, J=8.7, 5.0 Hz, 2H), 3.26 (s, 3H), 2.87-2.55 (m, 9H), 2.47-2.34 (m, 4H), 2.21-2.03 (m, 3H), 2.00-1.87 (m, 5H), 1.86-1.78 (m, 1H), 1.65-1.55 (m, 7H), 1.49-1.35 (m, 3H).

### Example 320: 3-cyano-N-(2-(trans)-4-((S)-4-(4-((R)-2,6-dioxopiperidin-3-yl)-3,5-difluorophenethyl)-3-(methoxymethyl)piperazin-1-yl)cyclohexyl)-6-(2-hydroxypropan-2-yl)-2H-indazol-5-yl)pyrrolo[1,2-b]pyridazine-7-carboxamide

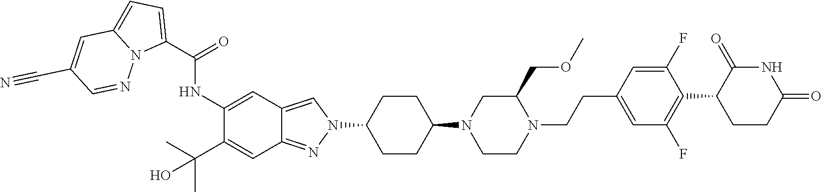 [SMILES: COC[C@@H]1CN([C@H]2CC[C@H](n3cc4cc(NC(=O)c5ccc6cc(C#N)cnn56)c(C(C)(C)O)cc4n3)CC2)CCN1CCc1cc(F)c([C@H]2CCC(=O)NC2=O)c(F)c1]

The title compound (8 mg, 10%) was prepared in a manner similar to that described in Example 319. ¹H NMR (500 MHz, DMSO) δ 12.02 (s, 1H), 10.94 (s, 1H), 8.93 (d, J=2.1 Hz, 1H), 8.74 (d, J=2.1 Hz, 1H), 8.55 (s, 1H), 8.33 (s, 1H), 8.21 (s, OH), 7.72 (d, J=4.8 Hz, 1H), 7.57 (s, 1H), 7.09 (d, J=4.8 Hz, 1H), 7.00 (d, J=10.0 Hz, 2H), 5.72 (s, 1H), 4.48-4.39 (m, 1H), 4.19 (dt, J=11.3, 5.7 Hz, 1H), 3.54-3.47 (m, 1H), 3.25 (s, 3H), 2.95-2.52 (m, 11H), 2.45-2.35 (m, 3H), 2.26-2.07 (m, 4H), 2.03-1.88 (m, 5H), 1.63 (s, 6H), 1.54-1.40 (m, 2H). [M+H]⁺=822.7.

### Example 355: 3-cyano-N-(2-(trans)-4-(4-(1-(4-((R)-2,6-dioxopiperidin-3-yl)-3,5-difluorophenyl)azetidine-3-carbonyl)piperazin-1-yl)cyclohexyl)-2H-[1,3]dioxolo[4,5-g]indazol-5-yl)pyrrolo[1,2-b]pyridazine-7-carboxamide

### Step 1: tert-butyl 4-(trans)-4-(5-bromo-2H-[1,3]dioxolo[4,5-g]indazol-2-yl)cyclohexyl)piperazine-1-carboxylate

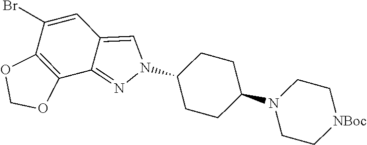 [SMILES: CC(C)(C)OC(=O)N1CCN([C@H]2CC[C@H](n3cc4cc(Br)c5c(c4n3)OCO5)CC2)CC1]

A solution of 7-bromo-4-nitrobenzo[d][1,3]dioxole-5-carbaldehyde (CAS: 1001433-41-5) (273 mg, 1.0 mmol) and tert-butyl 4-(trans)-4-aminocyclohexyl)piperazine-1-carboxylate (283 mg, 1.0 mmol) in IPA (10 mL) was stirred at 85° C. for 4 h. Then, the reaction was cooled down to room temperature. Bu₃P (606 mg, 3.0 mmol) was added to the reaction. The reaction was stirred at 85° C. for 12 h. The solvent was removed by reduced pressure. The residue was slurry with PE (3×15 mL) and filtered to afford product (360 mg, 71%). [M+H]⁺=507.4, 509.4.

### Step 2: tert-butyl 4-(trans)-4-(5-((diphenylmethylene)amino)-2H-[1,3]dioxolo[4,5-g]indazol-2-yl)cyclohexyl)piperazine-1-carboxylate

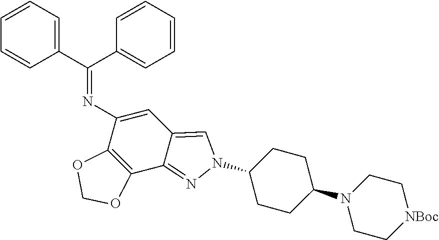 [SMILES: CC(C)(C)OC(=O)N1CCN([C@H]2CC[C@H](n3cc4cc(N=C(c5ccccc5)c5ccccc5)c5c(c4n3)OCO5)CC2)CC1]

To a solution of tert-butyl 4-(trans)-4-(5-bromo-2H-[1,3]dioxolo[4,5-g]indazol-2-yl)cyclohexyl)piperazine-1-carboxylate (360 mg, 0.71 mmol), diphenylmethanimine (129 mg, 0.71 mmol), Pd₂dba₃ (32.6 mg, 0.036 mmol), Xantphos (41.1 mg, 0.071 mmol) in Dioxane (10 mL) was added tBuONa (136.6 mg, 1.42 mmol). The reaction was stirred at 100° C. for 16 h. The mixture was then cooled. The mixture was concentrated under reduced pressure. The residue was washed by water (20 mL) and extracted with DCM (3×30 mL). The organic phase was dried over anhydrous sodium sulfate, filtered and concentrated under reduced pressure to afford the crude product (240 mg, 56%). [M+H]⁺=608.6.

### Step 3: tert-butyl 4-(trans)-4-(5-amino-2H-[1,3]dioxolo[4,5-g]indazol-2-yl)cyclohexyl)piperazine-1-carboxylate

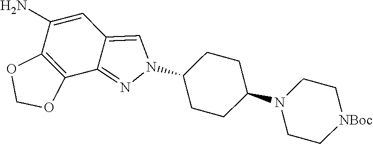 [SMILES: CC(C)(C)OC(=O)N1CCN([C@H]2CC[C@H](n3cc4cc(N)c5c(c4n3)OCO5)CC2)CC1]

A solution of tert-butyl 4-(trans)-4-(5-((diphenylmethylene)amino)-2H-[1,3]dioxolo[4,5-g]indazol-2-yl)cyclohexyl)piperazine-1-carboxylate (240 mg, 0.4 mmol) in water (5 mL) was dropwise HCl (2 mL, 1M, 2 mmol). The mixture was stirred at rt for 15 min. The mixture was washed by water (20 mL) and extracted with EtOAc (2×30 mL). The aqueous phase pH value was adjusted to 10-11 by saturated Na₂CO₃. The mixture was extracted with dichloromethane (3×30 mL). The organic phase was dried over anhydrous sodium sulfate, filtered and concentrated under reduced pressure to afford product (120 mg, 68%). [M+H]⁺=444.6.

### Step 4: tert-butyl 4-(trans)-4-(5-(3-cyanopyrrolo[1,2-b]pyridazine-7-carboxamido)-2H-[1,3]dioxolo[4,5-g]indazol-2-yl)cyclohexyl)piperazine-1-carboxylate

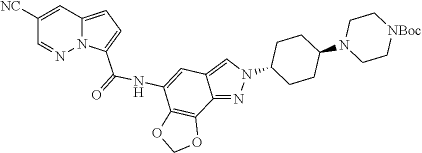 [SMILES: CC(C)(C)OC(=O)N1CCN([C@H]2CC[C@H](n3cc4cc(NC(=O)c5ccc6cc(C#N)cnn56)c5c(c4n3)OCO5)CC2)CC1]

To a solution of tert-butyl 4-(trans)-4-(5-amino-2H-[1,3]dioxolo[4,5-g]indazol-2-yl)cyclohexyl)piperazine-1-carboxylate (120 mg, 0.27 mmol), DIEA (104.5 mg, 0.81 mmol) and 3-cyanopyrrolo[1,2-b]pyridazine-7-carboxylic acid (50.5 mg, 0.27 mmol) in DMF (6 mL) was added HATU (113 mg, 0.3 mmol). The reaction was stirred at rt for 4 h. The solvent was removed by reduced pressure. The residue was washed water (15 mL), extracted with dichloromethane (3×20 mL), and washed with brine (30 mL). The organic phase was dried over anhydrous sodium sulfate, filtered and concentrated under reduced pressure to afford the crude product, which was purified with silica gel column chromatography (DCM:MeOH=100:0˜18:1 gradient elution) to give the product (150 mg, 91% yield). [M+H]⁺=613.5.

### Step 5: 3-cyano-N-(2-(trans)-4-(piperazin-1-yl)cyclohexyl)-2H-[1,3]dioxolo[4,5-g]indazol-5-yl)pyrrolo[1,2-b]pyridazine-7-carboxamide

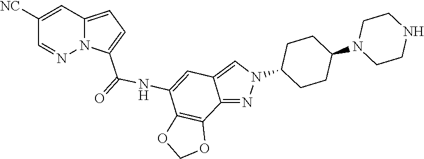 [SMILES: N#Cc1cnn2c(C(=O)Nc3cc4cn([C@H]5CC[C@H](N6CCNCC6)CC5)nc4c4c3OCO4)ccc2c1]

To a solution of tert-butyl 4-(trans)-4-(5-(3-cyanopyrrolo[1,2-b]pyridazine-7-carboxamido)-2H-[1,3]dioxolo[4,5-g]indazol-2-yl)cyclohexyl)piperazine-1-carboxylate (150 mg, 0.24 mmol) in DCM (5 mL) was dropwise TFA (1 mL). The reaction was stirred at rt for 3 h. The mixture pH value was adjusted to 10-11 by saturated Na₂CO₃. The mixture was washed water (15 mL), extracted with dichloromethane (3×20 mL) The organic phase was dried over anhydrous sodium sulfate, filtered and concentrated under reduced pressure to afford the product (101 mg, 81% yield). [M+H]⁺=513.6

### Step 6: 3-cyano-N-(2-(trans)-4-(4-(1-(4-((R)-2,6-dioxopiperidin-3-yl)-3,5-difluorophenyl)azetidine-3-carbonyl)piperazin-1-yl)cyclohexyl)-2H-[1,3]dioxolo[4,5-g]indazol-5-yl)pyrrolo[1,2-b]pyridazine-7-carboxamide

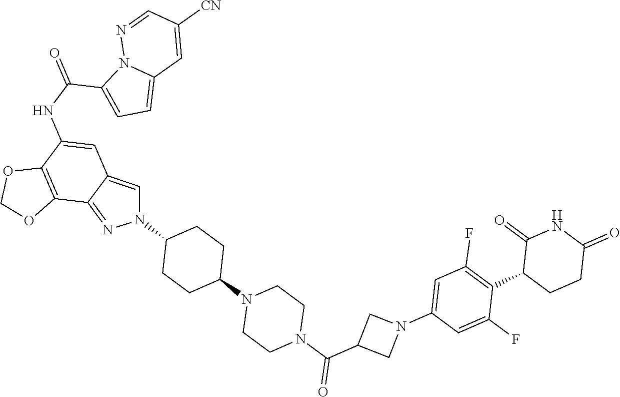 [SMILES: N#Cc1cnn2c(C(=O)Nc3cc4cn([C@H]5CC[C@H](N6CCN(C(=O)C7CN(c8cc(F)c([C@H]9CCC(=O)NC9=O)c(F)c8)C7)CC6)CC5)nc4c4c3OCO4)ccc2c1]

To a solution of 3-cyano-N-(2-((1r,4r)-4-(piperazin-1-yl)cyclohexyl)-2H-[1,3]dioxolo[4,5-g]indazol-5-yl)pyrrolo[1,2-b]pyridazine-7-carboxamide (25.6 mg, 0.05 mmol), DIEA (19 mg, 0.15 mmol) and (R)-1-(4-(2,6-dioxopiperidin-3-yl)-3,5-difluorophenyl)azetidine-3-carboxylic acid (16.2 mg, 0.05 mmol) in DMF (5 mL) was added T₃P (63.6 mg, 50%, 0.1 mmol). The reaction was stirred at rt for 4 h. The mixture was washed water (15 mL), extracted with dichloromethane (3×20 mL). The organic layers were combined and washed with brine (3×30 mL). The organic phase was dried over anhydrous sodium sulfate, filtered and concentrated under reduced pressure to afford the crude product, which was purified with silica gel column chromatography (DCM:MeOH=100:0˜9:1 gradient elution) to give the product (7 mg, 17% yield). [M+H]⁺=819.4. ¹H NMR (500 MHz, DMSO) δ 10.85 (s, 2H), 8.96 (d, J=41.7 Hz, 2H), 8.47 (s, 1H), 8.21 (s, 1H), 7.78 (d, J=4.8 Hz, 1H), 7.13 (d, J=4.9 Hz, 1H), 6.24 (s, 2H), 6.17 (d, J=11.3 Hz, 2H), 4.47-4.38 (m, 1H), 4.08-3.99 (m, 3H), 3.95-3.89 (m, 2H), 3.87-3.81 (m, 1H), 3.52-3.45 (m, 2H), 2.83-2.72 (m, 2H), 2.25-2.02 (m, 9H), 2.00-1.91 (m, 6H), 1.54-1.45 (m, 2H).

### Example 356: 3-cyano-N-(2-(trans)-4-(4-(1-(4-((R)-2,6-dioxopiperidin-3-yl)-3-methoxyphenyl)azetidine-3-carbonyl)piperazin-1-yl)cyclohexyl)-2H-[1,3]dioxolo[4,5-g]indazol-5-yl)pyrrolo[1,2-b]pyridazine-7-carboxamide

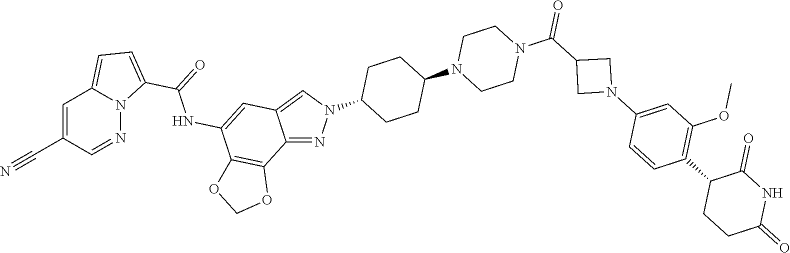 [SMILES: COc1cc(N2CC(C(=O)N3CCN([C@H]4CC[C@H](n5cc6cc(NC(=O)c7ccc8cc(C#N)cnn78)c7c(c6n5)OCO7)CC4)CC3)C2)ccc1[C@H]1CCC(=O)NC1=O]

The title compound (10 mg, 15%) was prepared in a manner similar to that described in Example 355. ¹H NMR (500 MHz, DMSO) δ 10.85 (s, 1H), 10.65 (s, 1H), 9.00 (d, J=1.9 Hz, 1H), 8.92 (s, 1H), 8.48 (d, J=7.9 Hz, 1H), 8.21 (s, 1H), 7.78 (d, J=4.7 Hz, 1H), 7.13 (d, J=4.7 Hz, 1H), 6.89 (d, J=8.1 Hz, 1H), 6.24 (s, 2H), 6.06 (d, J=7.9 Hz, 1H), 5.99 (d, J=7.7 Hz, 1H), 4.47-4.37 (m, 1H), 4.05-3.98 (m, 2H), 3.91-3.85 (m, 2H), 3.85-3.73 (m, 3H), 3.71 (s, 3H), 3.51-3.46 (m, 2H), 2.22-2.10 (m, 8H), 2.22-2.11 (m, 5H), 1.88-1.82 (m, 2H), 1.57-1.45 (m, 3H). [M+H]⁺=813.6.

### Example 358: 3-cyano-N-(2-((1S,4r)-4-((S)-4-((1-(4-((R)-2,6-dioxopiperidin-3-yl)-3,5-difluorophenyl)azetidin-3-yl)methyl)-3-(methoxymethyl)piperazin-1-yl)cyclohexyl)-6-(2-hydroxypropan-2-yl)-2H-indazol-5-yl)pyrrolo[1,2-b]pyridazine-7-carboxamide

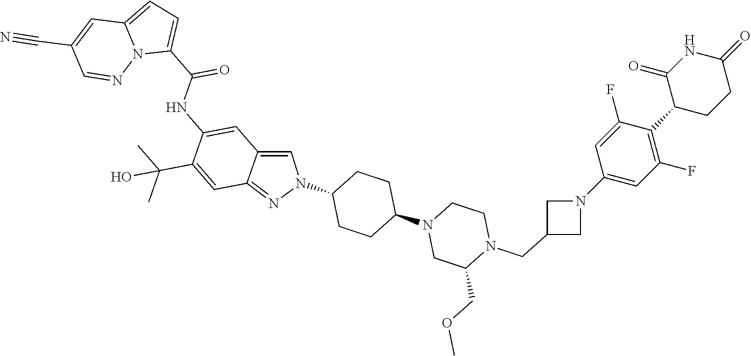 [SMILES: COC[C@@H]1CN([C@H]2CC[C@H](n3cc4cc(NC(=O)c5ccc6cc(C#N)cnn56)c(C(C)(C)O)cc4n3)CC2)CCN1CC1CN(c2cc(F)c([C@H]3CCC(=O)NC3=O)c(F)c2)C1]

The title compound (56.35 mg, 62%) was prepared in a manner similar to that described in Example 290. ¹H NMR (500 MHz, DMSO) δ 12.02 (s, 1H), 10.85 (s, 1H), 8.93 (d, J=5 Hz, 1H), 8.74 (d, J=5 Hz, 1H), 8.55 (s, 1H), 8.33 (s, 1H), 7.72 (d, J=5 Hz, 1H), 7.57 (s, 1H), 7.09 (d, J=5 Hz, 1H), 6.10 (d, J=10 Hz, 2H), 5.72 (s, 1H), 4.46-4.39 (m, 1H), 4.02 (dd, J=5 Hz, 10 Hz, 1H), 3.95-3.86 (m, 2H), 3.55-3.51 (m, 1H), 3.49-3.43 (m, 2H), 3.24 (s, 3H), 3.00-2.86 (m, 2H), 2.82-2.73 (m, 3H), 2.68-2.63 (m, 1H), 2.48-2.45 (m, 4H), 2.42-2.34 (m, 2H), 2.28-2.12 (m, 4H), 2.11-2.02 (m, 1H), 1.98-1.88 (m, 5H), 1.63 (s, 6H), 1.52-1.40 (m, 2H). [M+H]⁺=863.7.

### Example 387: 3-cyano-N-(2-((1S,4r)-4-((S)-4-(1-(4-((R)-2,6-dioxopiperidin-3-yl)-3,5-difluorophenyl)azetidin-3-yl)-3-(methoxymethyl)piperazin-1-yl)cyclohexyl)-6-(2-hydroxypropan-2-yl)-2H-indazol-5-yl)pyrrolo[1,2-b]pyridazine-7-carboxamide

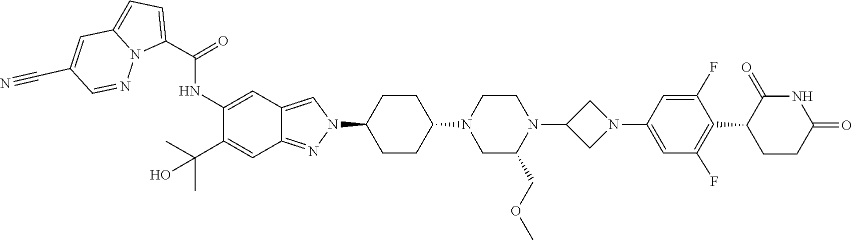 [SMILES: COC[C@@H]1CN([C@H]2CC[C@H](n3cc4cc(NC(=O)c5ccc6cc(C#N)cnn56)c(C(C)(C)O)cc4n3)CC2)CCN1C1CN(c2cc(F)c([C@H]3CCC(=O)NC3=O)c(F)c2)C1]

The title compound (120.06 mg, 57.64%) was prepared in a manner similar to that described in Example 290. ¹H NMR (500 MHz, DMSO) δ 12.02 (s, 1H), 10.85 (s, 1H), 8.93 (d, J=5 Hz, 1H), 8.74 (d, J=5 Hz, 1H), 8.55 (s, 1H), 8.32 (s, 1H), 7.72 (d, J=5 Hz, 1H), 7.57 (s, 1H), 7.09 (d, J=5 Hz, 1H), 6.13 (d, J=10 Hz, 2H), 5.72 (s, 1H), 4.46-4.39 (m, 1H), 4.02 (dd, J=5 Hz, 10 Hz, 1H), 3.95-3.86 (m, 2H), 3.73-3.57 (m, 3H), 3.52-3.48 (m, 1H), 3.38-3.34 (m, 1H), 3.26 (s, 3H), 2.82-2.73 (m, 1H), 2.71-2.63 (m, 2H), 2.60-2.52 (m, 5H), 2.42-2.31 (m, 2H), 2.20-2.02 (m, 3H), 1.98-1.88 (m, 5H), 1.63 (s, 6H), 1.52-1.40 (m, 2H). [M+H]⁺=849.8.

### Example 389: 3-cyano-N-(2-((1S,4r)-4-((S)-2-(cyanomethyl)-4-(4-((R)-2,6-dioxopiperidin-3-yl)-3,5-difluorophenethyl)piperazin-1-yl)cyclohexyl)-6-(2-hydroxypropan-2-yl)-2H-indazol-5-yl)pyrrolo[1,2-b]pyridazine-7-carboxamide

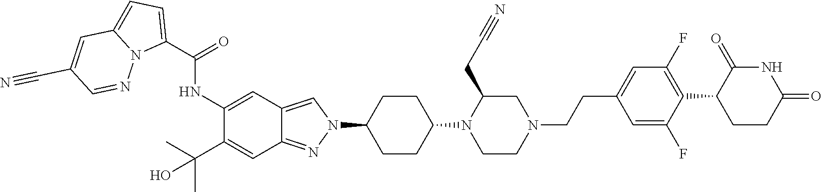 [SMILES: CC(C)(O)c1cc2nn([C@H]3CC[C@H](N4CCN(CCc5cc(F)c([C@H]6CCC(=O)NC6=O)c(F)c5)C[C@@H]4CC#N)CC3)cc2cc1NC(=O)c1ccc2cc(C#N)cnn12]

The title compound (19.92 mg, 23%) was prepared in a manner similar to that described in Example 290. ¹H NMR (500 MHz, DMSO) δ 12.03 (s, 1H), 10.95 (s, 1H), 8.94 (d, J=5 Hz, 1H), 8.74 (d, J=5 Hz, 1H), 8.55 (s, 1H), 8.32 (s, 1H), 7.72 (d, J=5 Hz, 1H), 7.57 (s, 1H), 7.09 (d, J=5 Hz, 1H), 7.04 (d, J=10 Hz, 2H), 5.72 (s, 1H), 4.46-4.38 (m, 1H), 4.20 (dd, J=5 Hz, 10 Hz, 1H), 3.08-3.00 (m, 1H), 2.94-2.88 (m, 1H), 2.85-2.62 (m, 7H), 2.60-2.52 (m, 5H), 2.37-2.30 (m, 2H), 2.21-2.08 (m, 3H), 2.07-1.82 (m, 5H), 1.69-1.58 (m, 7H), 1.46-1.38 (m, 1H). [M+H]⁺=817.7.

### Example 410: 3-cyano-N-(2-((1S,4r)-4-((S)-4-(1-(4-(2,4-dioxotetrahydropyrimidin-1(2H)-yl)-3,5-difluorophenyl)azetidine-3-carbonyl)-3-(methoxymethyl)piperazin-1-yl)cyclohexyl)-6-(2-hydroxypropan-2-yl)-2H-indazol-5-yl)pyrrolo[1,2-b]pyridazine-7-carboxamide

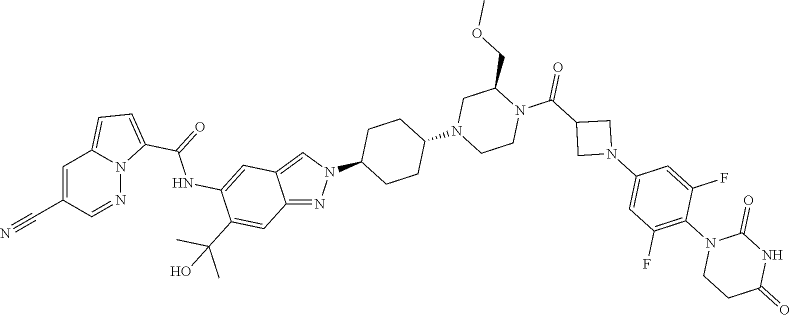 [SMILES: COC[C@@H]1CN([C@H]2CC[C@H](n3cc4cc(NC(=O)c5ccc6cc(C#N)cnn56)c(C(C)(C)O)cc4n3)CC2)CCN1C(=O)C1CN(c2cc(F)c(N3CCC(=O)NC3=O)c(F)c2)C1]

The title compound (104.24 mg, 67.75%) was prepared in a manner similar to that described in Example 178. ¹H NMR (500 MHz, DMSO) δ 12.02 (s, 1H), 10.50 (s, 1H), 8.93 (d, J=5 Hz, 1H), 8.74 (d, J=5 Hz, 1H), 8.55 (s, 1H), 8.33 (s, 1H), 7.72 (d, J=5 Hz, 1H), 7.57 (s, 1H), 7.09 (d, J=5 Hz, 1H), 6.25 (d, J=10 Hz, 2H), 5.72 (s, 1H), 4.58-4.54 (m, 0.5H), 4.47-4.38 (m, 1H), 4.25-4.20 (m, 0.5H), 4.12-4.03 (m, 1.5H), 3.99-3.82 (m, 4H), 3.71-3.57 (m, 3H), 3.54-3.48 (m, 0.5H), 3.46-3.37 (m, 1H), 3.30 (s, 1.5H), 3.26 (s, 1.5H), 3.15-3.07 (m, 0.5H), 2.97-2.77 (m, 2.5H), 2.68 (t, J=5 Hz, 2H), 2.46-2.40 (m, 2H), 2.38-2.14 (m, 4H), 2.00-1.85 (m, 4H), 1.63 (s, 6H), 1.55-1.40 (m, 2H). [M+H]⁺=878.7.

### Example 412: 7-(2-(1′-((R)-1-(4-((R)-2,6-dioxopiperidin-3-yl)-3,5-difluorophenyl)pyrrolidine-3-carbonyl)-[1,4′-bipiperidin]-4-yl)-8,8-dimethyl-2,8-dihydro-[1,3]oxazino[4,5-f]indazol-6-yl)pyrrolo[1,2-b]pyridazine-3-carbonitrile

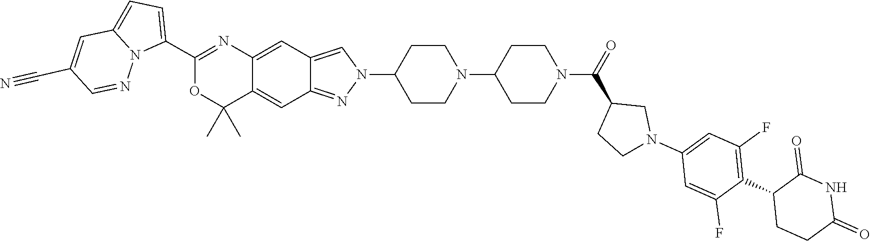 [SMILES: CC1(C)OC(c2ccc3cc(C#N)cnn23)=Nc2cc3cn(C4CCN(C5CCN(C(=O)[C@@H]6CCN(c7cc(F)c([C@H]8CCC(=O)NC8=O)c(F)c7)C6)CC5)CC4)nc3cc21]

The title compound (4.13 mg, 6.56%) was prepared in a manner similar to that described in Example 178. ¹H NMR (500 MHz, DMSO) δ 10.84 (s, 1H), 10.50 (s, 1H), 8.84 (d, J=5 Hz, 1H), 8.70 (d, J=5 Hz, 1H), 8.40 (s, 1H), 7.62-7.58 (m, 2H), 7.51 (s, 1H), 7.04 (d, J=5 Hz, 1H), 6.23 (t, J=10 Hz, 2H), 4.46-4.40 (m, 2H), 4.10-4.39 (m, 2H), 3.57-3.50 (m, 1H), 3.46-3.41 (m, 1H), 3.27-3.22 (m, 2H), 3.11-2.97 (m, 3H), 2.82-2.73 (m, 1H), 2.65-2.55 (m, 2H), 2.45-2.25 (m, 5H), 2.20-2.00 (m, 6H), 1.98-1.90 (m, 1H), 1.88-1.78 (m, 2H), 1.74 (s, 6H), 1.51-1.38 (m, 1H), 1.35-1.20 (m, 1H). [M+H]⁺=829.8.

### Example 106: 3-cyano-N-(2-((1R,4r)-4-(4-((R)-1-(4-(2,4-dioxotetrahydropyrimidin-1(2H)-yl)-3,5-difluorophenyl)pyrrolidine-3-carbonyl)piperazin-1-yl)cyclohexyl)-6-(2-hydroxypropan-2-yl)-2H-indazol-5-yl)pyrrolo[1,2-b]pyridazine-7-carboxamide

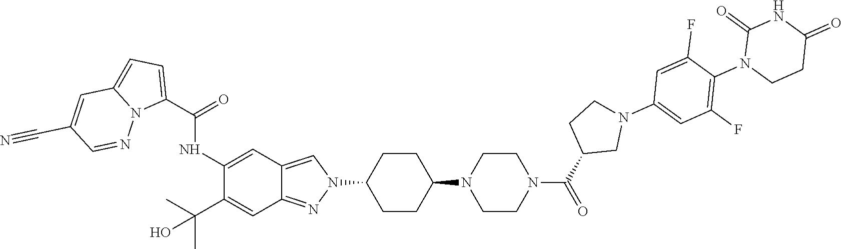 [SMILES: CC(C)(O)c1cc2nn([C@H]3CC[C@H](N4CCN(C(=O)[C@@H]5CCN(c6cc(F)c(N7CCC(=O)NC7=O)c(F)c6)C5)CC4)CC3)cc2cc1NC(=O)c1ccc2cc(C#N)cnn12]

The title compound (30 mg, 35.35%) was prepared in a manner similar to that described in Example 178. ¹H NMR (500 MHz, DMSO) δ 12.02 (s, 1H), 10.47 (s, 1H), 8.93 (d, J=2.0 Hz, 1H), 8.74 (d, J=2.0 Hz, 1H), 8.55 (s, 1H), 8.34 (s, 1H), 7.72 (d, J=5.0 Hz, 1H), 7.57 (s, 1H), 7.09 (d, 1H), 6.35-6.28 (m, 2H), 5.72 (s, 1H), 4.49-4.38 (m, 1H), 3.63-3.53 (m, 5H), 3.51-3.43 (m, 3H), 3.38-3.34 (m, 2H), 3.30-3.24 (m, 3H), 2.68 (t, J=6.5 Hz, 2H), 2.62-2.55 (m, 2H), 2.53-2.52 (m, 1H), 2.22-2.14 (m, 3H), 2.13-2.04 (m, 1H), 2.01-1.90 (m, 4H), 1.63 (s, 6H), 1.56-1.44 (m, 2H). [M+H]⁺=848.60.

### Example 186: 3-cyano-N-(2-((1S,4r)-4-((S)-4-(1-(4-((R)-2,6-dioxopiperidin-3-yl)-3,5-difluorophenyl)azetidine-3-carbonyl)-3-(methoxymethyl)piperazin-1-yl)cyclohexyl)-6-(2-hydroxypropan-2-yl)-2H-indazol-5-yl)pyrrolo[1,2-b]pyridazine-7-carboxamide

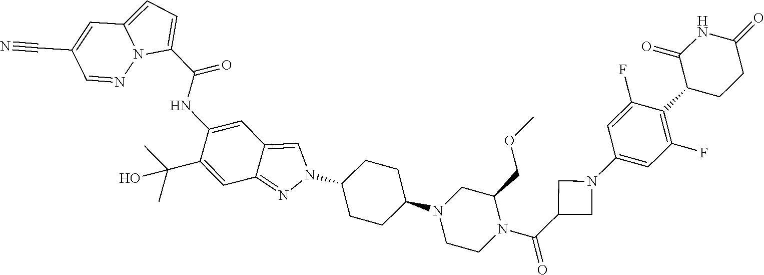 [SMILES: COC[C@@H]1CN([C@H]2CC[C@H](n3cc4cc(NC(=O)c5ccc6cc(C#N)cnn56)c(C(C)(C)O)cc4n3)CC2)CCN1C(=O)C1CN(c2cc(F)c([C@H]3CCC(=O)NC3=O)c(F)c2)C1]

The title compound (40 mg, 45.61%) was prepared in a manner similar to that described in Example 178. ¹H NMR (500 MHz, DMSO) δ 12.02 (s, 1H), 10.86 (s, 1H), 8.93 (d, J=2.0 Hz, 1H), 8.74 (d, J=2.0 Hz, 1H), 8.55 (s, 1H), 8.33 (s, 1H), 7.72 (d, J=5.0 Hz, 1H), 7.57 (s, 1H), 7.09 (d, J=5.0 Hz, 1H), 6.16 (dd, J=11.0, 4.0 Hz, 2H), 5.74 (s, 1H), 4.60-4.52 (m, 1H), 4.46-4.37 (m, 1H), 4.27-4.19 (m, 1H), 4.08-4.01 (m, 2H), 3.96-3.83 (m, 4H), 3.70-3.58 (m, 1H), 3.55-3.38 (m, 2H), 3.30 (s, 2H), 3.26 (s, 1H), 3.12 (t, J=11.5 Hz, 1H), 3.00-2.74 (m, 4H), 2.38-2.22 (m, 2H), 2.21-2.16 (m, 2H), 2.12-2.04 (m, 1H), 1.99-1.86 (m, 5H), 1.63 (s, 6H), 1.55-1.41 (m, 2H). [M+H]⁺=877.7.

### Example 197: 3-cyano-N-(2-((S)-1′-((1-(4-((R)-2,6-dioxopiperidin-3-yl)-3,5-difluorophenyl)azetidin-3-yl)methyl)-3′,3′-difluoro-[1,4′-bipiperidin]-4-yl)-6-(2-hydroxypropan-2-yl)-2H-indazol-5-yl)pyrrolo[1,2-b]pyridazine-7-carboxamide

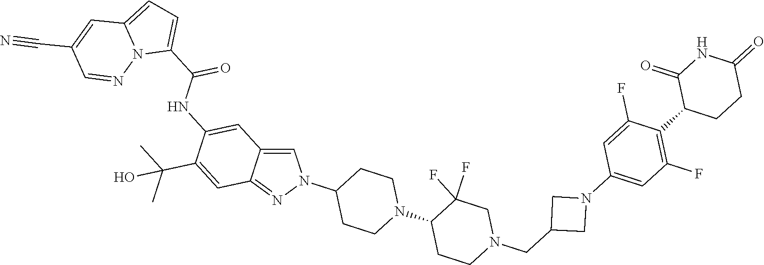 [SMILES: CC(C)(O)c1cc2nn(C3CCN([C@H]4CCN(CC5CN(c6cc(F)c([C@H]7CCC(=O)NC7=O)c(F)c6)C5)CC4(F)F)CC3)cc2cc1NC(=O)c1ccc2cc(C#N)cnn12]

The title compound (20 mg, 23.37%) was prepared in a manner similar to that described in Example 290. ¹H NMR (500 MHz, DMSO) δ 12.03 (s, 1H), 10.85 (s, 1H), 8.93 (d, J=2.0 Hz, 1H), 8.74 (d, J=2.0 Hz, 1H), 8.56 (s, 1H), 8.38 (s, 1H), 7.72 (d, J=5.0 Hz, 1H), 7.58 (s, 1H), 7.09 (d, J=5.0 Hz, 1H), 6.12 (d, J=11.0 Hz, 2H), 5.73 (s, 1H), 4.49-4.39 (m, 1H), 4.02 (t, J=6.5 Hz, 1H), 3.93 (t, J=7.5 Hz, 2H), 3.47 (s, 2H), 3.16-3.06 (m, 2H), 3.04-2.98 (m, 1H), 2.95-2.85 (m, 3H), 2.81-2.71 (m, 3H), 2.67-2.63 (m, 2H), 2.49-2.46 (m, 1H), 2.34-2.24 (m, 1H), 2.18-2.12 (m, 1H), 2.11-2.01 (m, 5H), 1.97-1.90 (m, 1H), 1.85-1.73 (m, 2H), 1.63 (s, 6H). [M+H]⁺=855.7.

### Example 390: 3-cyano-N-(2-((1r,4r)-4-(1-((1-(4-((R)-2,6-dioxopiperidin-3-yl)-3,5-difluorophenyl)azetidin-3-yl)methyl)piperidin-4-yl)cyclohexyl)-6-(2-hydroxypropan-2-yl)-2H-indazol-5-yl)pyrrolo[1,2-b]pyridazine-7-carboxamide

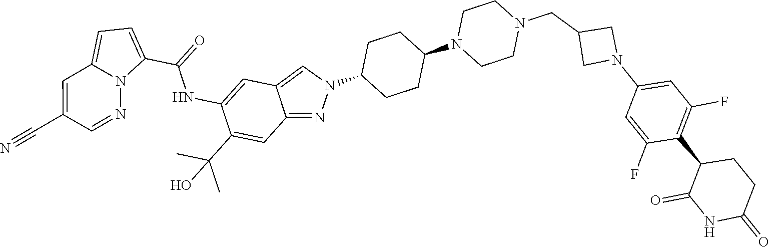 [SMILES: CC(C)(O)c1cc2nn([C@H]3CC[C@H](N4CCN(CC5CN(c6cc(F)c([C@H]7CCC(=O)NC7=O)c(F)c6)C5)CC4)CC3)cc2cc1NC(=O)c1ccc2cc(C#N)cnn12]

The title compound (46 mg, 56.23%) was prepared in a manner similar to that described in Example 290. ¹H NMR (500 MHz, DMSO) δ 12.02 (s, 1H), 10.85 (s, 1H), 8.93 (d, J=2.0 Hz, 1H), 8.74 (d, J=2.0 Hz, 1H), 8.55 (s, 1H), 8.33 (s, 1H), 7.72 (d, J=5.0 Hz, 1H), 7.57 (s, 1H), 7.09 (d, J=5.0 Hz, 1H), 6.10 (d, J=11.0 Hz, 2H), 5.71 (s, 1H), 4.40 (t, J=11.5 Hz, 1H), 4.02 (dd, J=12.5, 5.0 Hz, 1H), 3.93 (t, J=7.5 Hz, 2H), 3.47 (s, 2H), 3.31-3.28 (m, 1H), 2.98-2.73 (m, 4H), 2.56-2.52 (m, 1H), 2.49-2.46 (m, 1H), 2.20-2.12 (m, 2H), 2.10-2.03 (m, 1H), 1.98-1.81 (m, 7H), 1.69-1.59 (m, 8H), 1.26-1.17 (m, 5H), 1.09 (s, 1H). [M+H]⁺=818.6.

### Example 403: N-(2-((1r,4r)-4-(4-((1-(4-((R)-2,6-dioxopiperidin-3-yl)-3,5-difluorophenyl)azetidin-3-yl)methyl)piperazin-1-yl)cyclohexyl)-6-(2-hydroxypropan-2-yl)-2H-indazol-5-yl)furo[3,2-b]pyridine-3-carboxamide

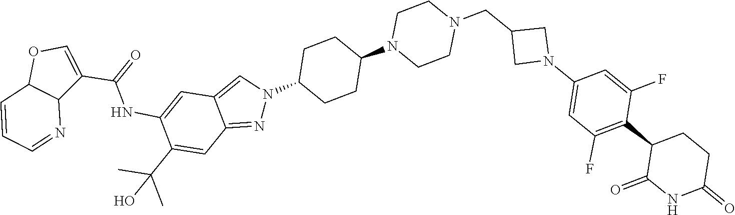 [SMILES: CC(C)(O)c1cc2nn([C@H]3CC[C@H](N4CCN(CC5CN(c6cc(F)c([C@H]7CCC(=O)NC7=O)c(F)c6)C5)CC4)CC3)cc2cc1NC(=O)C1=COC2C=CC=NC12]

The title compound (26 mg, 32.71%) was prepared in a manner similar to that described in Example 290. ¹H NMR (500 MHz, DMSO) δ 12.05 (s, 1H), 10.85 (s, 1H), 8.67 (d, J=4.0 Hz, 1H), 8.64 (s, 1H), 8.37 (s, 1H), 8.16 (d, J=8.5 Hz, 1H), 7.72 (s, 1H), 7.59 (s, 1H), 7.54 (dd, J=8.5, 5.0 Hz, 1H), 6.52 (s, 1H), 6.11 (d, J=11.0 Hz, 2H), 4.43 (t, J=11.5 Hz, 1H), 4.02 (dd, J=12.5, 5.0 Hz, 1H), 3.92 (t, J=7.5 Hz, 2H), 3.47 (t, J=5.5 Hz, 2H), 2.95-2.87 (m, 1H), 2.82-2.73 (m, 1H), 2.59-2.53 (m, 5H), 2.49-2.32 (m, 7H), 2.21-2.14 (m, 2H), 2.12-2.02 (m, 1H), 2.00-1.88 (m, 5H), 1.67 (s, 6H), 1.54-1.43 (m, 2H). [M+H]+=795.4.

### Example 404: N-(2-((1R,4r)-4-(4-(1-(4-((R)-2,6-dioxopiperidin-3-yl)-3,5-difluorophenyl)azetidin-3-yl)piperazin-1-yl)cyclohexyl)-6-(2-hydroxypropan-2-yl)-2H-indazol-5-yl)furo[3,2-b]pyridine-3-carboxamide

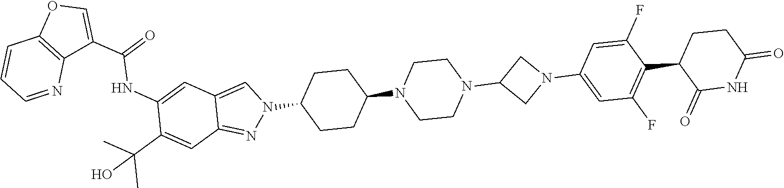 [SMILES: CC(C)(O)c1cc2nn([C@H]3CC[C@H](N4CCN(C5CN(c6cc(F)c([C@H]7CCC(=O)NC7=O)c(F)c6)C5)CC4)CC3)cc2cc1NC(=O)c1coc2cccnc12]

The title compound (19 mg, 20.21%) was prepared in a manner similar to that described in Example 290. ¹H NMR (500 MHz, DMSO) δ 12.05 (s, 1H), 10.87 (s, 1H), 8.67 (d, J=5.0 Hz, 1H), 8.64 (s, 1H), 8.37 (s, 1H), 8.24 (s, OH), 8.16 (d, J=8.5 Hz, 1H), 7.72 (s, 1H), 7.59 (s, 1H), 7.53 (dd, J=8.5, 4.5 Hz, 1H), 6.53 (s, 1H), 6.12 (d, J=11.0 Hz, 2H), 4.48-4.38 (m, 1H), 4.03 (dd, J=12.5, 5.0 Hz, 1H), 3.91 (t, J=7.5 Hz, 2H), 3.68-3.60 (m, 2H), 3.27-3.18 (m, 3H), 2.84-2.73 (m, 1H), 2.59-2.53 (m, 3H), 2.46-2.25 (m, 5H), 2.21-2.14 (m, 2H), 2.10-2.04 (m, 1H), 2.00-1.88 (m, 5H), 1.67 (s, 6H), 1.52-1.42 (m, 2H). [M+H]+=781.4.

### Example 405: N-(2-((1r,4r)-4-(4-(1-(4-((R)-2,6-dioxopiperidin-3-yl)-3,5-difluorophenyl)piperidin-4-yl)piperazin-1-yl)cyclohexyl)-6-(2-hydroxypropan-2-yl)-2H-indazol-5-yl)furo[3,2-b]pyridine-3-carboxamide

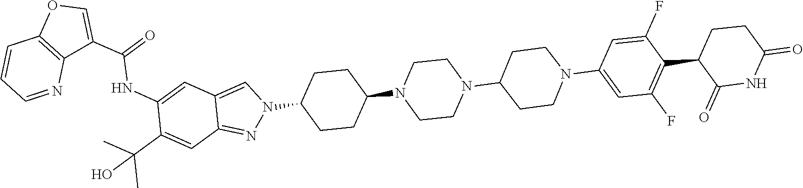 [SMILES: CC(C)(O)c1cc2nn([C@H]3CC[C@H](N4CCN(C5CCN(c6cc(F)c([C@H]7CCC(=O)NC7=O)c(F)c6)CC5)CC4)CC3)cc2cc1NC(=O)c1coc2cccnc12]

The title compound (23 mg, 28.43%) was prepared in a manner similar to that described in Example 290. ¹H NMR (500 MHz, DMSO) δ 11.98 (s, 1H), 10.80 (s, 1H), 8.60 (d, J=4.5 Hz, 1H), 8.57 (s, 1H), 8.30 (s, 1H), 8.13 (s, 1H), 8.09 (d, J=8.5 Hz, 1H), 7.65 (s, 1H), 7.53 (s, 1H), 7.47 (dd, J=8.5, 5.0 Hz, 1H), 6.61-6.51 (m, 2H), 4.34 (t, J=11.5 Hz, 1H), 3.98 (dd, J=12.5, 5.0 Hz, 1H), 3.70 (d, J=12.0 Hz, 2H), 2.78-2.61 (m, 3H), 2.51-2.44 (m, 8H), 2.32-2.23 (m, 2H), 2.13-2.05 (m, 2H), 2.04-1.96 (m, 1H), 1.93-1.80 (m, 5H), 1.78-1.70 (m, 2H), 1.57 (s, 6H), 1.45-1.27 (m, 4H). [M+H]⁺=809.3.

### Example 409: 3-cyano-N-(2-((1r,4r)-4-(1′-(4-((R)-2,6-dioxopiperidin-3-yl)-3,5-difluorophenyl)-[1,4′-bipiperidin]-4-yl)cyclohexyl)-6-(2-hydroxypropan-2-yl)-2H-indazol-5-yl)pyrrolo[1,2-b]pyridazine-7-carboxamide

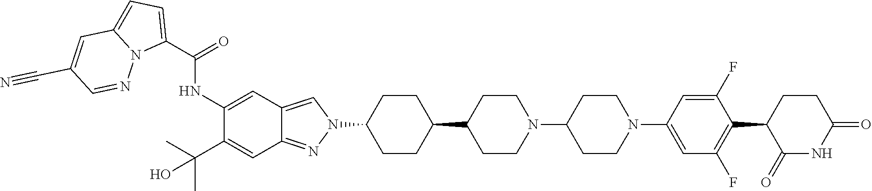 [SMILES: CC(C)(O)c1cc2nn([C@H]3CC[C@H](C4CCN(C5CCN(c6cc(F)c([C@H]7CCC(=O)NC7=O)c(F)c6)CC5)CC4)CC3)cc2cc1NC(=O)c1ccc2cc(C#N)cnn12]

The title compound (4 mg, 4.81%) was prepared in a manner similar to that described in Example 290. ¹H NMR (500 MHz, DMSO) δ 12.02 (s, 1H), 10.87 (s, 1H), 8.93 (d, J=2.0 Hz, 1H), 8.74 (d, J=2.0 Hz, 1H), 8.55 (s, 1H), 8.33 (s, 1H), 7.71 (d, J=5.0 Hz, 1H), 7.57 (s, 1H), 7.09 (d, J=5.0 Hz, 1H), 6.70-6.57 (m, 2H), 5.72 (s, 1H), 4.40 (s, 1H), 4.04 (dd, J=13.0, 5.0 Hz, 1H), 3.84-3.75 (m, 2H), 3.30-3.26 (m, 2H), 2.97-2.90 (m, 2H), 2.82-2.69 (m, 5H), 2.19-2.06 (m, 5H), 1.99-1.83 (m, 6H), 1.82-1.76 (m, 2H), 1.72-1.66 (m, 2H), 1.63 (s, 6H), 1.51-1.44 (m, 2H), 1.14-1.02 (m, 2H), 0.87-0.79 (m, 2H). [M+H]+=832.6.

### Example 153: (R)—N-(2-(3-(4-(1-(4-(2,6-dioxopiperidin-3-yl)-3,5-difluorophenyl)azetidine-3-carbonyl)piperazin-1-yl)bicyclo[1.1.1]pentan-1-yl)-6-(2-hydroxypropan-2-yl)-2H-indazol-5-yl)-6-(trifluoromethyl)picolinamide

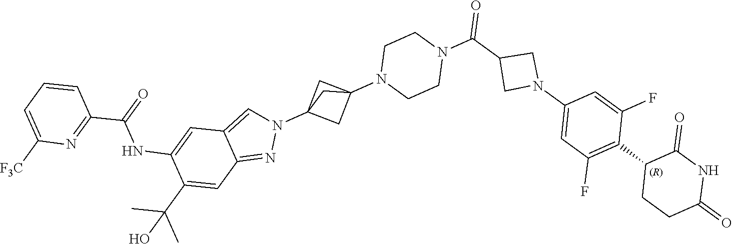

The title compound (4 mg, 4.81%) was prepared in a manner similar to that described in Example 178. ¹H NMR (500 MHz, DMSO) δ 12.40 (s, 1H), 10.86 (s, 1H), 8.74 (s, 1H), 8.45 (d, J=7.7 Hz, 1H), 8.40-8.33 (m, 2H), 8.16 (d, J=7.9 Hz, 1H), 7.61 (s, 1H), 6.18 (d, J=11.0 Hz, 2H), 5.99 (s, 1H), 4.09-4.01 (m, 4H), 3.98-3.90 (m, 3H), 3.89-3.81 (m, 2H), 3.58-3.53 (m, 3H), 3.41-3.37 (m, 4H), 2.35 (s, 6H), 2.14-2.00 (m, 1H), 1.97-1.92 (m, 1H), 1.62 (s, 6H). [M+H]⁺=821.8.

### Example 152: N-(2-(3-(4-((R)-1-(4-((R)-2,6-dioxopiperidin-3-yl)-3,5-difluorophenyl)pyrrolidine-3-carbonyl)piperazin-1-yl)bicyclo[1.1.1]pentan-1-yl)-6-(2-hydroxypropan-2-yl)-2H-indazol-5-yl)-6-(trifluoromethyl)picolinamide

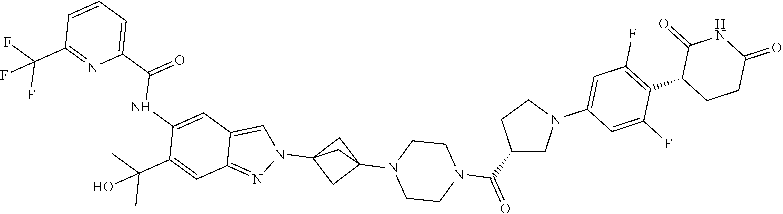 [SMILES: CC(C)(O)c1cc2nn(C34CC(N5CCN(C(=O)[C@@H]6CCN(c7cc(F)c([C@H]8CCC(=O)NC8=O)c(F)c7)C6)CC5)(C3)C4)cc2cc1NC(=O)c1cccc(C(F)(F)F)n1]

The title compound was prepared in a manner similar to that described in Example 178. ¹H NMR (500 MHz, DMSO) δ 12.40 (s, 1H), 10.84 (s, 1H), 8.74 (s, 1H), 8.45 (d, J=7.9 Hz, 1H), 8.39-8.34 (m, 2H), 8.16 (d, J=7.7 Hz, 1H), 7.61 (s, 1H), 6.23 (d, J=12.2 Hz, 2H), 5.98 (d, J=11.9 Hz, 1H), 4.07-3.98 (m, 1H), 3.66-3.60 (m, 2H), 3.56 (t, J=9.3 Hz, 3H), 3.50-3.43 (m, 2H), 3.38-3.33 (m, 2H), 3.30-3.23 (m, 4H), 2.82-2.74 (m, 1H), 2.36 (s, 4H), 2.21-2.04 (m, 5H), 1.98-1.92 (m, 1H), 1.74 (s, 1H), 1.62 (s, 6H). [M+H]⁺=835.7.

### Example 108: 3-cyano-N-(2-((1r,4r)-4-(4-((1-(4-(2,4-dioxotetrahydropyrimidin-1(2H)-yl)-3,5-difluorophenyl)azetidin-3-yl)methyl)piperazin-1-yl)cyclohexyl)-6-(2-hydroxypropan-2-yl)-2H-indazol-5-yl)pyrrolo[1,2-b]pyridazine-7-carboxamide

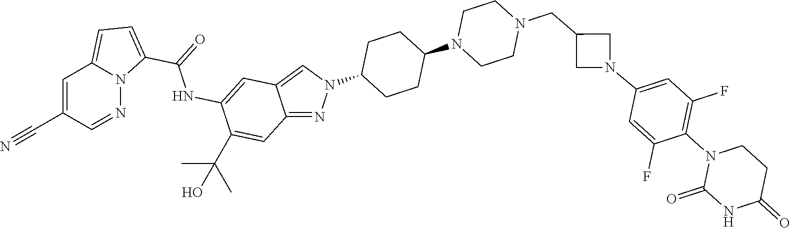 [SMILES: CC(C)(O)c1cc2nn([C@H]3CC[C@H](N4CCN(CC5CN(c6cc(F)c(N7CCC(=O)NC7=O)c(F)c6)C5)CC4)CC3)cc2cc1NC(=O)c1ccc2cc(C#N)cnn12]

The title compound (48 mg, 32%) was prepared in a manner similar to that described in Example 290. ¹H NMR (500 MHz, DMSO) δ 12.03 (s, 1H), 10.49 (s, 1H), 8.94 (d, J=5 Hz, 1H), 8.74 (d, J=5 Hz, 1H), 8.55 (s, 1H), 8.34 (s, 1H), 7.73 (d, J=5 Hz, 1H), 7.57 (s, 1H), 7.10 (d, J=5 Hz, 1H), 6.19 (d, J=10 Hz, 2H), 5.73 (s, 1H), 4.45-4.40 (m, 1H), 3.97-3.94 (m, 2H), 3.60-3.57 (m, 2H), 3.51-3.49 (m, 2H), 2.94-2.91 (m, 1H), 2.70-2.67 (m, 2H), 2.56-2.55 (m, 6H), 2.45-2.37 (m, 5H), 2.18-2.17 (m, 2H), 1.98-1.91 (m, 4H), 1.64 (s, 6H), 1.51-1.47 (m, 2H). [M+H]⁺=820.9.

### Example 348: 3-cyano-N-(2-((1r,4r)-4-(4-(3-(1-(2,6-dioxopiperidin-3-yl)-3-methyl-2-oxo-2,3-dihydro-1H-benzo[d]imidazol-4-yl)propyl)piperazin-1-yl)cyclohexyl)-6-(2-hydroxypropan-2-yl)-2H-indazol-5-yl)pyrrolo[1,2-b]pyridazine-7-carboxamide

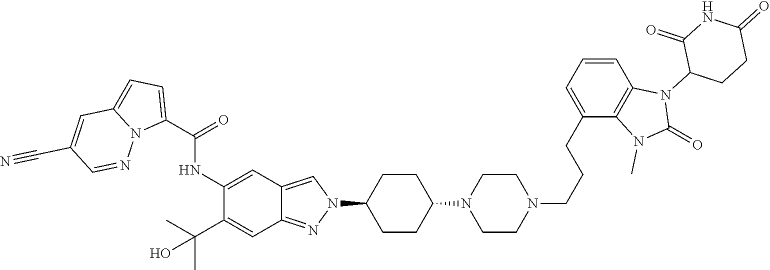 [SMILES: Cn1c(=O)n(C2CCC(=O)NC2=O)c2cccc(CCCN3CCN([C@H]4CC[C@H](n5cc6cc(NC(=O)c7ccc8cc(C#N)cnn78)c(C(C)(C)O)cc6n5)CC4)CC3)c21]

The title compound (60 mg, 55%) was prepared in a manner similar to that described in Example 290. ¹H NMR (500 MHz, DMSO) δ 12.02 (s, 1H), 11.09 (s, 1H), 8.94 (d, J=5 Hz, 1H), 8.74 (d, J=5 Hz, 1H), 8.55 (s, 1H), 8.33 (s, 1H), 7.72 (d, J=5 Hz, 1H), 7.57 (s, 1H), 7.09 (d, J=5 Hz, 1H), 6.96-6.89 (m, 3H), 5.72 (s, 1H), 4.45-4.40 (m, 1H), 3.58-3.56 (m, 4H), 2.93-2.90 (m, 3H), 2.70-2.60 (m, 4H), 2.45-2.36 (m, 7H), 2.18-2.16 (m, 2H), 2.01-1.85 (m, 6H), 1.78-1.70 (m, 2H), 1.63 (s, 6H), 1.53-1.40 (m, 2H). [M+H]⁺=826.6.

### Example 359: 3-cyano-N-(2-((1S,4r)-4-((3aS,6aS)-5-((1-(4-((R)-2,6-dioxopiperidin-3-yl)-3,5-difluorophenyl)azetidin-3-yl)methyl)hexahydropyrrolo[3,4-c]pyrrol-2(1H)-yl)cyclohexyl)-6-(2-hydroxypropan-2-yl)-2H-indazol-5-yl)pyrrolo[1,2-b]pyridazine-7-carboxamide

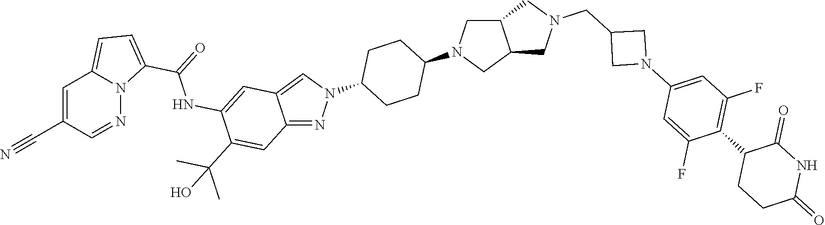

The title compound (128 mg, 42%) was prepared in a manner similar to that described in Example 290. ¹H NMR (500 MHz, DMSO) δ 12.03 (s, 1H), 10.85 (s, 1H), 8.94 (d, J=5 Hz, 1H), 8.74 (d, J=5 Hz, 1H), 8.56 (s, 1H), 8.34 (s, 1H), 7.72 (d, J=5 Hz, 1H), 7.57 (s, 1H), 7.09 (d, J=5 Hz, 1H), 6.10 (d, J=10.0 Hz, 2H), 5.72 (s, 1H), 4.48-4.43 (m, 1H), 4.04-4.01 (m, 1H), 3.91-3.88 (m, 2H), 3.49-3.48 (m, 3H), 3.01-2.96 (m, 2H), 2.94-2.88 (m, 2H), 2.84-2.72 (m, 5H), 2.67-2.63 (m, 2H), 2.55-2.49 (m, 3H), 2.20-1.91 (m, 9H), 1.63 (s, 6H), 1.53-1.42 (m, 2H). [M+H]⁺=845.7.

### Example 360: 3-cyano-N-(2-((1R,4r)-4-((3aR,6aR)-5-(1-(4-((R)-2,6-dioxopiperidin-3-yl)-3,5-difluorophenyl)azetidin-3-yl)hexahydropyrrolo[3,4-c]pyrrol-2(1H)-yl)cyclohexyl)-6-(2-hydroxypropan-2-yl)-2H-indazol-5-yl)pyrrolo[1,2-b]pyridazine-7-carboxamide

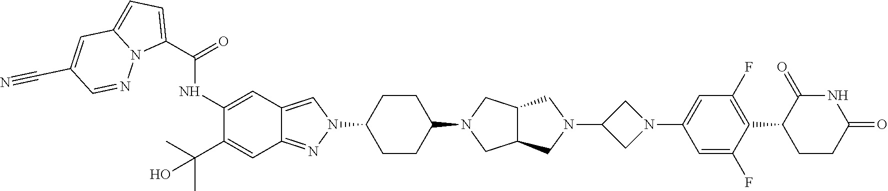

The title compound (134 mg, 45%) was prepared in a manner similar to that described in Example 290. ¹H NMR (500 MHz, DMSO) δ 12.02 (s, 1H), 10.85 (s, 1H), 8.94 (d, J=5 Hz, 1H), 8.74 (d, J=5 Hz, 1H), 8.55 (s, 1H), 8.34 (s, 1H), 7.72 (d, J=5 Hz, 1H), 7.57 (s, 1H), 7.09 (d, J=5 Hz, 1H), 6.11 (d, J=10.0 Hz, 2H), 5.72 (s, 1H), 4.48-4.43 (m, 1H), 4.04-4.01 (m, 1H), 3.92-3.83 (m, 3H), 3.68-3.66 (m, 2H), 2.93-2.74 (m, 5H), 2.67-2.54 (m, 5H), 2.14-1.93 (m, 11H), 1.63 (s, 6H), 1.47-1.42 (m, 2H). [M+H]⁺=831.7.

### Example 361: 3-cyano-N-(2-((1R,4r)-4-((3aR,6aR)-5-(1-(4-((R)-2,6-dioxopiperidin-3-yl)-3,5-difluorophenyl)piperidin-4-yl)hexahydropyrrolo[3,4-c]pyrrol-2(1H)-yl)cyclohexyl)-6-(2-hydroxypropan-2-yl)-2H-indazol-5-yl)pyrrolo[1,2-b]pyridazine-7-carboxamide

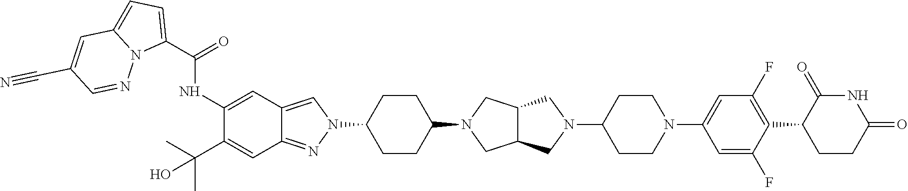

The title compound (108 mg, 35%) was prepared in a manner similar to that described in Example 290. ¹H NMR (500 MHz, DMSO) δ 12.03 (s, 1H), 10.87 (s, 1H), 8.94 (d, J=5 Hz, 1H), 8.74 (d, J=5 Hz, 1H), 8.55 (s, 1H), 8.34 (s, 1H), 7.72 (d, J=5 Hz, 1H), 7.57 (s, 1H), 7.09 (d, J=5 Hz, 1H), 6.63 (d, J=10.0 Hz, 2H), 5.72 (s, 1H), 4.48-4.43 (m, 1H), 4.06-4.03 (m, 1H), 3.73-3.71 (m, 2H), 2.95-2.90 (m, 4H), 2.81-2.71 (m, 6H), 2.64-2.54 (m, 4H), 2.15-1.82 (m, 12H), 1.63 (s, 6H), 1.49-1.41 (m, 4H). [M+H]⁺=859.7.

### Example 362: 3-cyano-N-(2-((1S,4r)-4-((3aS,6aS)-5-(4-((R)-2,6-dioxopiperidin-3-yl)-3,5-difluorophenethyl)hexahydropyrrolo[3,4-c]pyrrol-2(1H)-yl)cyclohexyl)-6-(2-hydroxypropan-2-yl)-2H-indazol-5-yl)pyrrolo[1,2-b]pyridazine-7-carboxamide

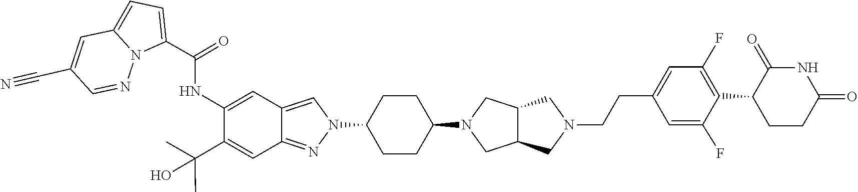 [SMILES: CC(C)(O)c1cc2nn([C@H]3CC[C@H](N4C[C@@H]5CN(CCc6cc(F)c([C@H]7CCC(=O)NC7=O)c(F)c6)C[C@H]5C4)CC3)cc2cc1NC(=O)c1ccc2cc(C#N)cnn12]

The title compound (35 mg, 12%) was prepared in a manner similar to that described in Example 290. ¹H NMR (500 MHz, DMSO) δ 12.02 (s, 1H), 10.95 (s, 1H), 8.94 (d, J=5 Hz, 1H), 8.74 (d, J=5 Hz, 1H), 8.55 (s, 1H), 8.34 (s, 1H), 7.72 (d, J=5 Hz, 1H), 7.57 (s, 1H), 7.09 (d, J=5 Hz, 1H), 7.02 (d, J=10.0 Hz, 2H), 5.73 (s, 1H), 4.48-4.43 (m, 1H), 4.21-4.18 (m, 1H), 2.91-2.77 (m, 7H), 2.74-2.52 (m, 8H), 2.16-1.91 (m, 10H), 1.63 (s, 6H), 1.48-1.42 (m, 2H). [M+H]⁺=804.7.

### Example 363: 3-cyano-N-(2-((1S,4r)-4-(4-((1S,3S)-3-(4-((R)-2,6-dioxopiperidin-3-yl)-3,5-difluorophenyl)cyclobutyl)piperazin-1-yl)cyclohexyl)-6-(2-hydroxypropan-2-yl)-2H-indazol-5-yl)pyrrolo[1,2-b]pyridazine-7-carboxamide

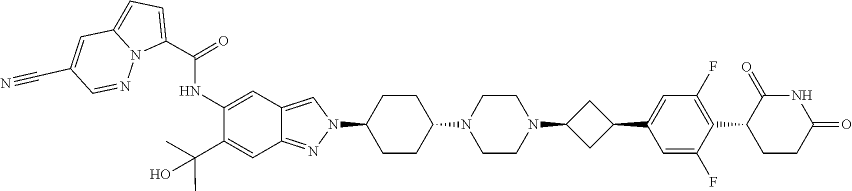 [SMILES: CC(C)(O)c1cc2nn([C@H]3CC[C@H](N4CCN([C@H]5C[C@@H](c6cc(F)c([C@H]7CCC(=O)NC7=O)c(F)c6)C5)CC4)CC3)cc2cc1NC(=O)c1ccc2cc(C#N)cnn12]

The title compound (128 mg, 53%) was prepared in a manner similar to that described in Example 290. ¹H NMR (500 MHz, DMSO) δ 12.03 (s, 1H), 10.95 (s, 1H), 8.93 (d, J=5 Hz, 1H), 8.74 (d, J=5 Hz, 1H), 8.55 (s, 1H), 8.32 (s, 1H), 7.72 (d, J=5 Hz, 1H), 7.57 (s, 1H), 7.09 (d, J=5 Hz, 1H), 6.97 (d, J=10.0 Hz, 2H), 5.73 (s, 1H), 4.44-4.41 (m, 1H), 4.22-4.18 (m, 1H), 3.17-3.14 (m, 1H), 2.81-2.77 (m, 1H), 2.68-2.66 (m, 1H), 2.56-2.52 (m, 4H), 2.43-2.37 (m, 7H), 2.17-2.11 (m, 3H), 2.01-1.90 (m, 6H), 1.86-1.80 (m, 2H), 1.63 (s, 6H), 1.50-1.46 (m, 2H). [M+H]⁺=804.7.

### Example 364: 3-cyano-N-(2-((1R,4r)-4-(4-((1R,3R)-3-(4-((R)-2,6-dioxopiperidin-3-yl)-3,5-difluorophenyl)cyclobutyl)piperazin-1-yl)cyclohexyl)-6-(2-hydroxypropan-2-yl)-2H-indazol-5-yl)pyrrolo[1,2-b]pyridazine-7-carboxamide

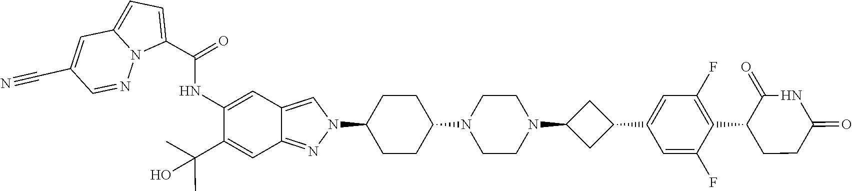 [SMILES: CC(C)(O)c1cc2nn([C@H]3CC[C@H](N4CCN([C@H]5C[C@H](c6cc(F)c([C@H]7CCC(=O)NC7=O)c(F)c6)C5)CC4)CC3)cc2cc1NC(=O)c1ccc2cc(C#N)cnn12]

The title compound (25 mg, 10%) was prepared in a manner similar to that described in Example 290. ¹H NMR (500 MHz, DMSO) δ 12.02 (s, 1H), 10.95 (s, 1H), 8.94 (d, J=5 Hz, 1H), 8.74 (d, J=5 Hz, 1H), 8.55 (s, 1H), 8.33 (s, 1H), 7.72 (d, J=5 Hz, 1H), 7.57 (s, 1H), 7.10 (d, J=5 Hz, 1H), 7.05 (d, J=10.0 Hz, 2H), 5.72 (s, 1H), 4.44-4.41 (m, 1H), 4.22-4.19 (m, 1H), 3.48-3.46 (m, 1H), 2.85-2.78 (m, 2H), 2.64-2.55 (m, 4H), 2.43-2.31 (m, 7H), 2.19-2.12 (m, 6H), 2.01-1.91 (m, 5H), 1.63 (s, 6H), 1.52-1.48 (m, 2H). [M+H]⁺=804.7.

### Example 365: 3-cyano-N-(2-((1R,4r)-4-((R)-4-((1S,3S)-3-(4-((R)-2,6-dioxopipendin-3-yl)-3,5-difluorophenyl)cyclobutyl)-2-methylpiperazin-1-yl)cyclohexyl)-6-(2-hydroxypropan-2-yl)-2H-indazol-5-yl)pyrrolo[1,2-b]pyridazine-7-carboxamide

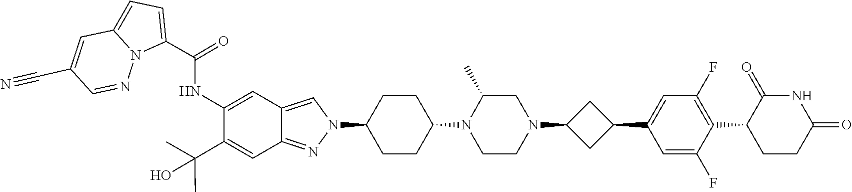 [SMILES: C[C@@H]1CN([C@H]2C[C@@H](c3cc(F)c([C@H]4CCC(=O)NC4=O)c(F)c3)C2)CCN1[C@H]1CC[C@H](n2cc3cc(NC(=O)c4ccc5cc(C#N)cnn45)c(C(C)(C)O)cc3n2)CC1]

The title compound (10 mg, 11%) was prepared in a manner similar to that described in Example 290. ¹H NMR (500 MHz, DMSO) δ 12.02 (s, 1H), 10.95 (s, 1H), 8.94 (d, J=5 Hz, 1H), 8.74 (d, J=5 Hz, 1H), 8.55 (s, 1H), 8.33 (s, 1H), 7.72 (d, J=5 Hz, 1H), 7.57 (s, 1H), 7.10 (d, J=5 Hz, 1H), 6.98 (d, J=10.0 Hz, 2H), 5.72 (s, 1H), 4.43-4.41 (m, 1H), 4.22-4.18 (m, 1H), 3.18-3.14 (m, 1H), 2.89-2.77 (m, 3H), 2.64-2.63 (m, 3H), 2.55-2.54 (m, 2H), 2.43-2.36 (m, 3H), 2.20-2.09 (m, 3H), 2.01-1.84 (m, 7H), 1.75-1.70 (m, 2H), 1.63 (s, 6H), 1.45-1.39 (m, 2H). [M+H]⁺=818.6.

### Example 366: 3-cyano-N-(2-((1R,4r)-4-((R)-4-((1R,3R)-3-(4-((R)-2,6-dioxopiperidin-3-yl)-3,5-difluorophenyl)cyclobutyl)-2-methylpiperazin-1-yl)cyclohexyl)-6-(2-hydroxypropan-2-yl)-2H-indazol-5-yl)pyrrolo[1,2-b]pyridazine-7-carboxamide

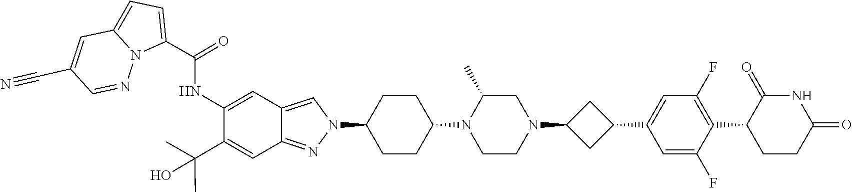 [SMILES: C[C@@H]1CN([C@H]2C[C@H](c3cc(F)c([C@H]4CCC(=O)NC4=O)c(F)c3)C2)CCN1[C@H]1CC[C@H](n2cc3cc(NC(=O)c4ccc5cc(C#N)cnn45)c(C(C)(C)O)cc3n2)CC1]

The title compound (1.53 mg, 1.7%) was prepared in a manner similar to that described in Example 290. ¹H NMR (500 MHz, DMSO) δ 12.02 (s, 1H), 10.95 (s, 1H), 8.94 (d, J=5 Hz, 1H), 8.74 (d, J=5 Hz, 1H), 8.55 (s, 1H), 8.34 (s, 1H), 7.72 (d, J=5 Hz, 1H), 7.57 (s, 1H), 7.10 (d, J=5 Hz, 1H), 7.05 (d, J=10.0 Hz, 2H), 5.72 (s, 1H), 4.44-4.41 (m, 1H), 4.22-4.19 (m, 1H), 3.48-3.46 (m, 1H), 2.90-2.78 (m, 3H), 2.75-2.63 (m, 2H), 2.56-2.52 (m, 3H), 2.37-2.31 (m, 3H), 2.20-2.12 (m, 5H), 2.01-1.91 (m, 5H), 1.75-1.72 (m, 2H), 1.63 (s, 6H), 1.45-1.39 (m, 2H). [M+H]⁺=818.6.

### Example 367: 3-cyano-N-(2-((1R,4r)-4-((R)-4-((1-(4-((R)-2,6-dioxopiperidin-3-yl)-3,5-difluorophenyl)azetidin-3-yl)methyl)-3-(methoxymethyl)piperazin-1-yl)cyclohexyl)-6-(2-hydroxypropan-2-yl)-2H-indazol-5-yl)pyrrolo[1,2-b]pyridazine-7-carboxamide

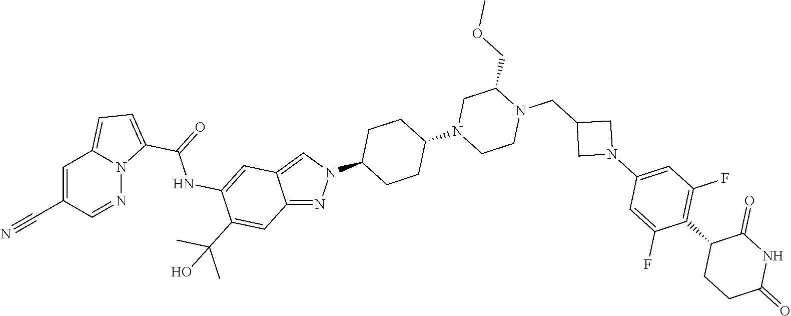 [SMILES: COC[C@H]1CN([C@H]2CC[C@H](n3cc4cc(NC(=O)c5ccc6cc(C#N)cnn56)c(C(C)(C)O)cc4n3)CC2)CCN1CC1CN(c2cc(F)c([C@H]3CCC(=O)NC3=O)c(F)c2)C1]

The title compound (169 mg, 56%) was prepared in a manner similar to that described in Example 290. ¹H NMR (500 MHz, DMSO) δ 12.02 (s, 1H), 10.85 (s, 1H), 8.94 (d, J=5 Hz, 1H), 8.74 (d, J=5 Hz, 1H), 8.55 (s, 1H), 8.33 (s, 1H), 7.72 (d, J=5 Hz, 1H), 7.57 (s, 1H), 7.10 (d, J=5 Hz, 1H), 6.11 (d, J=10.0 Hz, 2H), 5.72 (s, 1H), 4.43-4.40 (m, 1H), 4.04-4.01 (m, 1H), 3.94-3.88 (m, 2H), 3.52-3.43 (m, 3H), 3.24 (s, 3H), 2.98-2.89 (m, 2H), 2.81-2.74 (m, 3H), 2.66-2.64 (m, 1H), 2.49-2.45 (m, 4H), 2.40-2.36 (m, 2H), 2.28-2.16 (m, 4H), 2.09-2.05 (m, 1H), 1.95-1.92 (m, 5H), 1.63 (s, 6H), 1.49-1.44 (m, 2H). [M+H]⁺=863.7.

### Example 369: 3-cyano-N-(2-((1S,4r)-4-((S)-4-(4-((R)-2,6-dioxopiperidin-3-yl)-3,5-difluorophenethyl)-2-(2-hydroxy-2-methylpropyl)piperazin-1-yl)cyclohexyl)-6-(2-hydroxypropan-2-yl)-2H-indazol-5-yl)pyrrolo[1,2-b]pyridazine-7-carboxamide

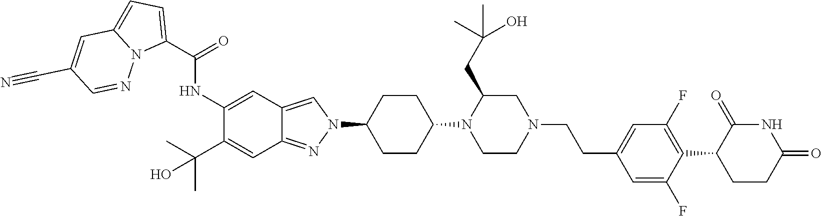 [SMILES: CC(C)(O)C[C@H]1CN(CCc2cc(F)c([C@H]3CCC(=O)NC3=O)c(F)c2)CCN1[C@H]1CC[C@H](n2cc3cc(NC(=O)c4ccc5cc(C#N)cnn45)c(C(C)(C)O)cc3n2)CC1]

The title compound (12 mg, 28%) was prepared in a manner similar to that described in Example 290. ¹H NMR (500 MHz, DMSO) δ 12.02 (s, 1H), 10.94 (s, 1H), 8.94 (d, J=5 Hz, 1H), 8.74 (d, J=5 Hz, 1H), 8.55 (s, 1H), 8.33 (s, 1H), 7.72 (d, J=5 Hz, 1H), 7.57 (s, 1H), 7.10 (d, J=5 Hz, 1H), 7.04 (d, J=10.0 Hz, 2H), 5.72 (s, 1H), 4.43-4.40 (m, 1H), 4.21-4.18 (m, 1H), 3.01-2.98 (m, 1H), 2.84-2.73 (m, 5H), 2.64-2.54 (m, 4H), 2.47-2.29 (m, 4H), 2.18-2.12 (m, 4H), 2.02-1.90 (m, 4H), 1.80-1.78 (m, 1H), 1.63 (s, 6H), 1.58-1.51 (m, 1H), 1.43-1.34 (m, 2H), 1.14 (s, 6H). [M+H]⁺=850.7.

### Example 382: 3-cyano-N-(2-((1R,4r)-4-(4-(((1R,3R)-3-(4-((R)-2,6-dioxopiperidin-3-yl)-3,5-difluorophenyl)cyclobutyl)methyl)piperazin-1-yl)cyclohexyl)-6-(2-hydroxypropan-2-yl)-2H-indazol-5-yl)pyrrolo[1,2-b]pyridazine-7-carboxamide

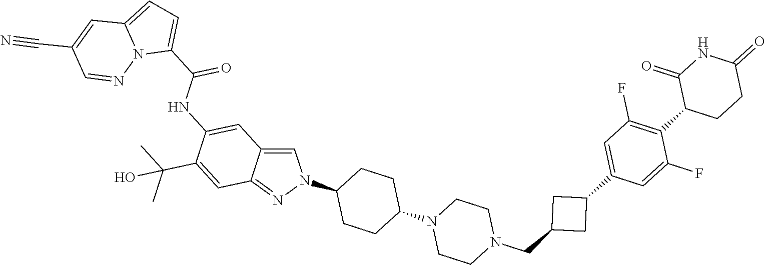 [SMILES: CC(C)(O)c1cc2nn([C@H]3CC[C@H](N4CCN(C[C@H]5C[C@H](c6cc(F)c([C@H]7CCC(=O)NC7=O)c(F)c6)C5)CC4)CC3)cc2cc1NC(=O)c1ccc2cc(C#N)cnn12]

The title compound (43 mg, 51%) was prepared in a manner similar to that described in Example 290. ¹H NMR (500 MHz, DMSO) δ 12.03 (s, 1H), 10.95 (s, 1H), 8.94 (d, J=5 Hz, 1H), 8.74 (d, J=5 Hz, 1H), 8.55 (s, 1H), 8.34 (s, 1H), 7.72 (d, J=5 Hz, 1H), 7.57 (s, 1H), 7.10 (d, J=5 Hz, 1H), 7.03 (d, J=10.0 Hz, 2H), 5.72 (s, 1H), 4.45-4.40 (m, 1H), 4.22-4.19 (m, 1H), 3.62-3.55 (m, 1H), 2.85-2.78 (m, 1H), 2.64-2.54 (m, 9H), 2.47-2.37 (m, 4H), 2.23-2.09 (m, 7H), 2.01-1.91 (m, 5H), 1.63 (s, 6H), 1.52-1.45 (m, 2H). [M+H]⁺=818.4.

### Example 388: 3-cyano-N-(2-((1S,4r)-4-((S)-4-(((S)-1-(4-((R)-2,6-dioxopiperidin-3-yl)-3,5-difluorophenyl)pyrrolidin-3-yl)methyl)-2-(methoxymethyl)piperazin-1-yl)cyclohexyl)-6-(2-hydroxypropan-2-yl)-2H-indazol-5-yl)pyrrolo[1,2-b]pyridazine-7-carboxamide

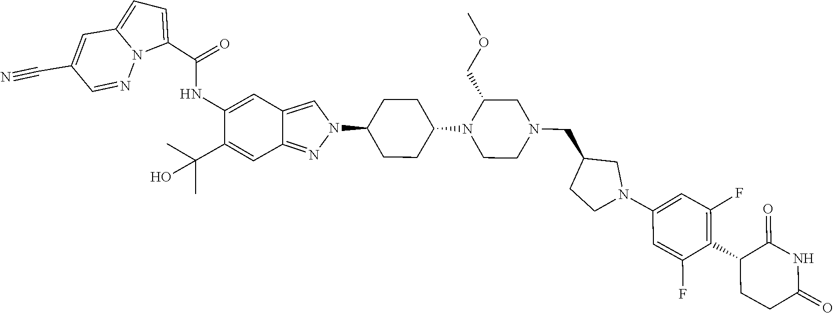 [SMILES: COC[C@@H]1CN(C[C@@H]2CCN(c3cc(F)c([C@H]4CCC(=O)NC4=O)c(F)c3)C2)CCN1[C@H]1CC[C@H](n2cc3cc(NC(=O)c4ccc5cc(C#N)cnn45)c(C(C)(C)O)cc3n2)CC1]

The title compound (76 mg, 49%) was prepared in a manner similar to that described in Example 290. ¹H NMR (500 MHz, DMSO) δ 12.02 (s, 1H), 10.84 (s, 1H), 8.94 (d, J=5 Hz, 1H), 8.74 (d, J=5 Hz, 1H), 8.55 (s, 1H), 8.34 (s, 1H), 7.72 (d, J=5 Hz, 1H), 7.57 (s, 1H), 7.10 (d, J=5 Hz, 1H), 6.18 (d, J=10.0 Hz, 2H), 5.72 (s, 1H), 4.43-4.39 (m, 1H), 4.03-3.99 (m, 1H), 3.53-3.51 (m, 1H), 3.38-3.35 (m, 3H), 3.28-3.19 (m, 5H), 2.96-2.93 (m, 1H), 2.85-2.74 (m, 4H), 2.65-2.63 (m, 1H), 2.55-2.48 (m, 3H), 2.28-2.27 (m, 2H), 2.18-2.16 (m, 3H), 2.07-1.90 (m, 7H), 1.80-1.66 (m, 3H), 1.63 (s, 6H), 1.43-1.40 (m, 1H). [M+H]⁺=877.6.

### Example 392: 3-cyano-N-(2-((1S,4r)-4-(4-(((S)-1-(4-((R)-2,6-dioxopiperidin-3-yl)-3,5-difluorophenyl)pyrrolidin-3-yl)methyl)piperazin-1-yl)cyclohexyl)-6-(2-hydroxypropan-2-yl)-2H-indazol-5-yl)pyrrolo[1,2-b]pyridazine-7-carboxamide

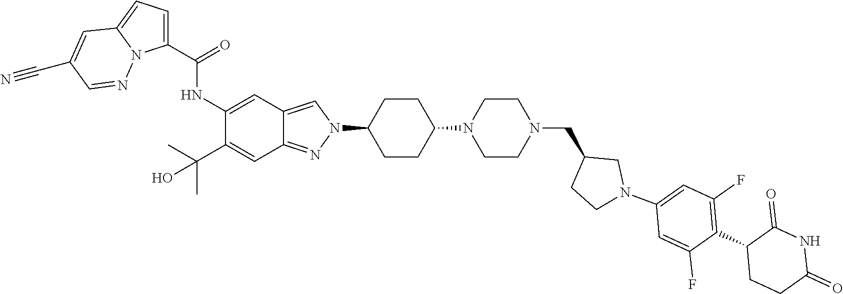 [SMILES: CC(C)(O)c1cc2nn([C@H]3CC[C@H](N4CCN(C[C@@H]5CCN(c6cc(F)c([C@H]7CCC(=O)NC7=O)c(F)c6)C5)CC4)CC3)cc2cc1NC(=O)c1ccc2cc(C#N)cnn12]

The title compound (103 mg, 65%) was prepared in a manner similar to that described in Example 290. ¹H NMR (500 MHz, DMSO) δ 12.02 (s, 1H), 10.84 (s, 1H), 8.93 (d, J=5 Hz, 1H), 8.74 (d, J=5 Hz, 1H), 8.55 (s, 1H), 8.33 (s, 1H), 7.72 (d, J=5 Hz, 1H), 7.57 (s, 1H), 7.09 (d, J=5 Hz, 1H), 6.18 (d, J=10.0 Hz, 2H), 5.72 (s, 1H), 4.45-4.40 (m, 1H), 4.03-3.99 (m, 1H), 3.28-3.17 (m, 4H), 2.96-2.93 (m, 1H), 2.78-2.74 (m, 1H), 2.57-2.51 (m, 5H), 2.44-2.36 (m, 5H), 2.31-2.30 (m, 2H), 2.18-2.16 (m, 2H), 2.10-2.05 (m, 2H), 1.98-1.91 (m, 5H), 1.72-1.66 (m, 1H), 1.63 (s, 6H), 1.51-1.45 (m, 2H). [M+H]⁺=833.8.

### Example 396: 3-cyano-N-(2-((1R,4r)-4-(4-((3R)-1-(4-(2,6-dioxopiperidin-3-yl)-3,5-difluorophenyl)pyrrolidin-3-yl)piperazin-1-yl)cyclohexyl)-6-(2-hydroxypropan-2-yl)-2H-indazol-5-yl)pyrrolo[1,2-b]pyridazine-7-carboxamide

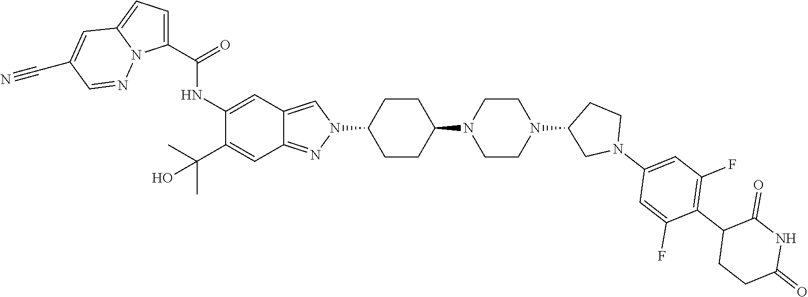 [SMILES: CC(C)(O)c1cc2nn([C@H]3CC[C@H](N4CCN([C@@H]5CCN(c6cc(F)c(C7CCC(=O)NC7=O)c(F)c6)C5)CC4)CC3)cc2cc1NC(=O)c1ccc2cc(C#N)cnn12]

The title compound (14 mg, 18%) was prepared in a manner similar to that described in Example 290. ¹H NMR (500 MHz, DMSO) δ 12.02 (s, 1H), 10.84 (s, 1H), 8.94 (d, J=5 Hz, 1H), 8.74 (d, J=5 Hz, 1H), 8.55 (s, 1H), 8.33 (s, 1H), 7.72 (d, J=5 Hz, 1H), 7.57 (s, 1H), 7.10 (d, J=5 Hz, 1H), 6.23 (d, J=10.0 Hz, 2H), 5.72 (s, 1H), 4.45-4.40 (m, 1H), 4.03-4.00 (m, 1H), 3.47-3.45 (m, 1H), 3.37-3.35 (m, 3H), 3.23-3.19 (m, 1H), 3.04-3.01 (m, 1H), 2.89-2.74 (m, 2H), 2.60-2.54 (m, 4H), 2.46-2.36 (m, 3H), 2.18-2.16 (m, 3H), 2.10-2.05 (m, 2H), 1.98-1.91 (m, 5H), 1.82-1.78 (m, 1H), 1.63 (s, 6H), 1.51-1.45 (m, 2H). [M+H]⁺=819.8.

### Example 413: 3-cyano-N-(2-((1R,4r)-4-(4-(4-((R)-2,6-dioxopiperidin-3-yl)-3,5-difluorophenethyl)piperazin-1-yl)cyclohexyl)-6-(2-hydroxypropan-2-yl)-2H-indazol-5-yl)pyrrolo[1,2-a]pyrimidine-8-carboxamide

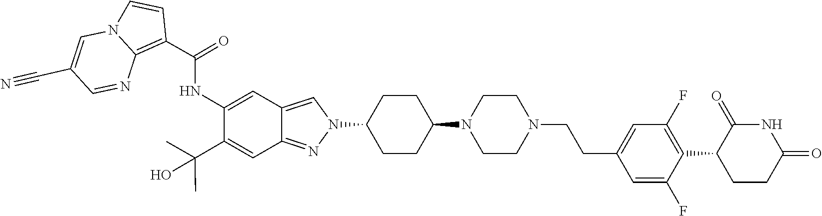 [SMILES: CC(C)(O)c1cc2nn([C@H]3CC[C@H](N4CCN(CCc5cc(F)c([C@H]6CCC(=O)NC6=O)c(F)c5)CC4)CC3)cc2cc1NC(=O)c1ccn2cc(C#N)cnc12]

The title compound (3 mg, 10%) was prepared in a manner similar to that described in Example 290. ¹H NMR (500 MHz, DMSO) δ 11.37 (s, 1H), 10.95 (s, 1H), 8.51 (d, J=5 Hz, 1H), 8.38 (s 1H), 8.30 (s, 1H), 7.83 (d, J=5 Hz, 1H), 7.57 (s, 1H), 7.76 (d, J=5 Hz, 1H), 7.62 (d, J=5 Hz, 1H), 7.55 (s, 1H), 7.03 (d, J=10.0 Hz, 2H), 5.64 (s, 1H), 4.43-4.40 (m, 1H), 4.21-4.18 (m, 1H), 3.51-3.41 (m, 3H), 2.83-2.74 (m, 3H), 2.54-2.51 (m, 6H), 2.49-2.36 (m, 3H), 2.18-2.11 (m, 3H), 2.01-1.93 (m, 5H), 1.63 (s, 6H), 1.50-1.45 (m, 2H). [M+H]⁺=778.6.

### Example 112: 3-cyano-N-(2-((1R,4r)-4-(4-(((R)-1-(4-(2,4-dioxotetrahydropyrimidin-1(2H)-yl)-3,5-difluorophenyl)pyrrolidin-3-yl)methyl)piperazin-1-yl)cyclohexyl)-6-(2-hydroxypropan-2-yl)-2H-indazol-5-yl)pyrrolo[1,2-b]pyridazine-7-carboxamide

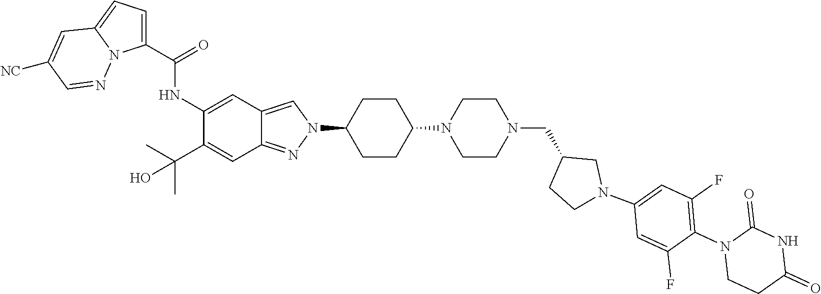 [SMILES: CC(C)(O)c1cc2nn([C@H]3CC[C@H](N4CCN(C[C@H]5CCN(c6cc(F)c(N7CCC(=O)NC7=O)c(F)c6)C5)CC4)CC3)cc2cc1NC(=O)c1ccc2cc(C#N)cnn12]

The title compound was prepared in a manner similar to that described in Example 290. 1H NMR (500 MHz, DMSO) δ 11.95 (s, 1H), 10.40 (s, 1H), 8.86 (d, J=2.1 Hz, 1H), 8.67 (d, J=2.1 Hz, 1H), 8.48 (s, 1H), 8.27 (s, 1H), 7.65 (d, J=4.8 Hz, 1H), 7.50 (s, 1H), 7.02 (d, J=4.8 Hz, 1H), 6.20 (d, J=11.3 Hz, 2H), 5.66 (s, 1H), 4.36 (t, J=11.3 Hz, 1H), 3.50 (dd, J=18.3, 11.6 Hz, 2H), 3.22-3.09 (m, 4H), 2.89 (dd, J=9.5, 6.7 Hz, 1H), 2.61 (t, J=6.7 Hz, 2H), 2.55-2.47 (m, 5H), 2.32 (dd, J=28.0, 6.4 Hz, 4H), 2.24 (d, J=7.4 Hz, 2H), 2.10 (d, J=10.5 Hz, 2H), 2.00 (dd, J=11.7, 5.0 Hz, 1H), 1.88 (dd, J=23.5, 12.0 Hz, 4H), 1.64 (dt, J=18.4, 6.9 Hz, 1H), 1.56 (s, 6H), 1.47-1.33 (m, 2H). [M+H]⁺=834.4.

### Example 357: 3-cyano-N-(2-((1R,4r)-4-(4-(3-(4-((R)-2,6-dioxopiperidin-3-yl)-3,5-difluorophenyl)propyl)piperazin-1-yl)cyclohexyl)-6-(2-hydroxypropan-2-yl)-2H-indazol-5-yl)pyrrolo[1,2-b]pyridazine-7-carboxamide

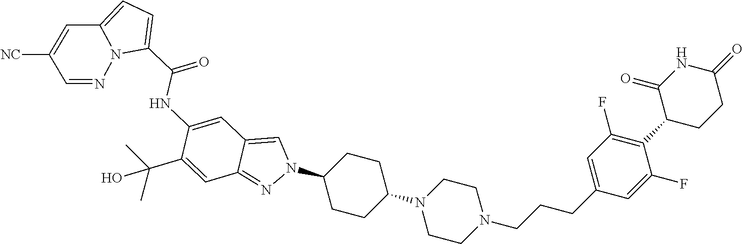 [SMILES: CC(C)(O)c1cc2nn([C@H]3CC[C@H](N4CCN(CCCc5cc(F)c([C@H]6CCC(=O)NC6=O)c(F)c5)CC4)CC3)cc2cc1NC(=O)c1ccc2cc(C#N)cnn12]

The title compound (20 mg, 24.6%) was prepared in a manner similar to that described in Example 290. ¹H NMR (500 MHz, DMSO) δ 12.02 (s, 1H), 10.94 (s, 1H), 8.93 (d, J=1.8 Hz, 1H), 8.73 (d, J=1.7 Hz, 1H), 8.55 (s, 1H), 8.33 (s, 1H), 7.72 (d, J=4.7 Hz, 1H), 7.57 (s, 1H), 7.09 (d, J=4.8 Hz, 1H), 6.99 (d, J=10.2 Hz, 2H), 5.72 (s, 1H), 4.42 (t, J=11.7 Hz, 1H), 4.19 (dd, J=12.6, 5.1 Hz, 1H), 2.85-2.76 (m, 1H), 2.65-2.52 (m, 7H), 2.45-2.31 (m, 5H), 2.30-2.25 (m, 2H), 2.21-2.06 (m, 3H), 2.04-1.88 (m, 5H), 1.77-1.69 (m, 2H), 1.63 (s, 6H), 1.54-1.39 (m, 2H). [M+H]⁺=792.6.

### Example 345: 2-((1r,4r)-4-(4-((1-(4-((R)-2,6-dioxopiperidin-3-yl)-3,5-difluorophenyl)azetidin-3-yl)methyl)piperazin-1-yl)cyclohexyl)-N-(imidazo[1,2-b]pyridazin-3-yl)-6-methoxy-2H-indazole-5-carboxamide

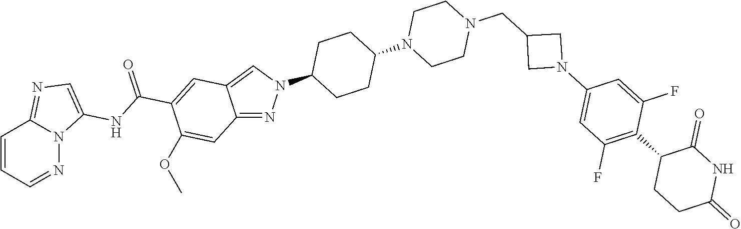 [SMILES: COc1cc2nn([C@H]3CC[C@H](N4CCN(CC5CN(c6cc(F)c([C@H]7CCC(=O)NC7=O)c(F)c6)C5)CC4)CC3)cc2cc1C(=O)Nc1cnc2cccnn12]

The title compound (16 mg, 18.7%) was prepared in a manner similar to that described in Example 290. ¹H NMR (500 MHz, DMSO) δ 11.05 (s, 1H), 10.85 (s, 1H), 8.64 (dd, J=4.4, 1.4 Hz, 1H), 8.58 (s, 2H), 8.15 (dd, J=9.2, 1.4 Hz, 1H), 8.05 (s, 1H), 7.26 (s, 1H), 7.22 (dd, J=9.2, 4.4 Hz, 1H), 6.10 (d, J=11.1 Hz, 2H), 5.76 (s, OH), 4.45 (t, J=11.5 Hz, 1H), 4.12 (s, 3H), 4.02 (dd, J=12.6, 5.0 Hz, 1H), 3.93 (t, J=7.4 Hz, 2H), 3.47 (t, J=5.8 Hz, 2H), 2.91 (dt, J=13.3, 6.5 Hz, 1H), 2.83-2.73 (m, 1H), 2.61-2.52 (m, 7H), 2.37 (dd, J=10.8, 8.9 Hz, 5H), 2.19 (d, J=9.9 Hz, 2H), 2.07 (dt, J=13.2, 9.3 Hz, 1H), 1.94 (dd, J=22.6, 11.6 Hz, 5H), 1.49 (q, J=11.6 Hz, 2H). [M+H]⁺=767.6.

### Example 349: (R)-3-cyano-N-(2-(3-(4-(2-(1-(4-(2,6-dioxopiperidin-3-yl)-3,5-difluorophenyl)piperidin-4-yl)ethyl)piperazin-1-yl)bicyclo[1.1.1]pentan-1-yl)-6-(2-hydroxypropan-2-yl)-2H-indazol-5-yl)pyrrolo[1,2-b]pyridazine-7-carboxamide

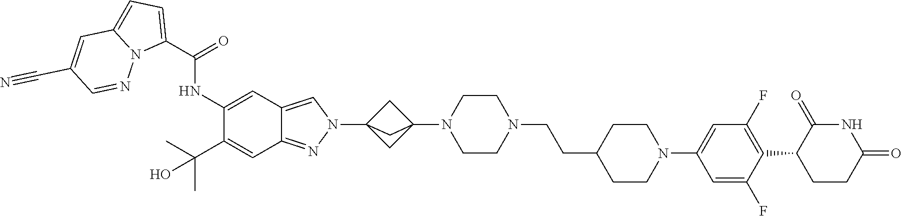 [SMILES: CC(C)(O)c1cc2nn(C34CC(N5CCN(CCC6CCN(c7cc(F)c([C@H]8CCC(=O)NC8=O)c(F)c7)CC6)CC5)(C3)C4)cc2cc1NC(=O)c1ccc2cc(C#N)cnn12]

The title compound (29 mg, 35.6%) was prepared in a manner similar to that described in Example 290. ¹H NMR (500 MHz, DMSO) δ 12.05 (s, 1H), 10.86 (s, 1H), 8.93 (d, J=1.9 Hz, 1H), 8.73 (d, J=1.8 Hz, 1H), 8.59 (s, 1H), 8.35 (s, 1H), 7.72 (d, J=4.7 Hz, 1H), 7.60 (s, 1H), 7.09 (d, J=4.7 Hz, 1H), 6.60 (d, J=12.9 Hz, 2H), 5.78 (s, 1H), 4.04 (dd, J=12.3, 4.8 Hz, 1H), 3.74 (d, J=12.3 Hz, 3H), 2.87-2.61 (m, 6H), 2.45-2.23 (m, 12H), 2.13-2.02 (m, 1H), 1.99-1.92 (m, 1H), 1.72 (d, J=11.2 Hz, 3H), 1.63 (s, 6H), 1.49 (brs, 1H), 1.42-1.34 (m, 2H), 1.23-1.12 (m, 2H). [M+H]⁺=845.8.

### Example 350: 3-cyano-N-(2-(3-(4-(3-(1-(2,6-dioxopiperidin-3-yl)-3-methyl-2-oxo-2,3-dihydro-1H-benzo[d]imidazol-4-yl)propyl)piperazin-1-yl)bicyclo[1.1.1]pentan-1-yl)-6-(2-hydroxypropan-2-yl)-2H-indazol-5-yl)pyrrolo[1,2-b]pyridazine-7-carboxamide

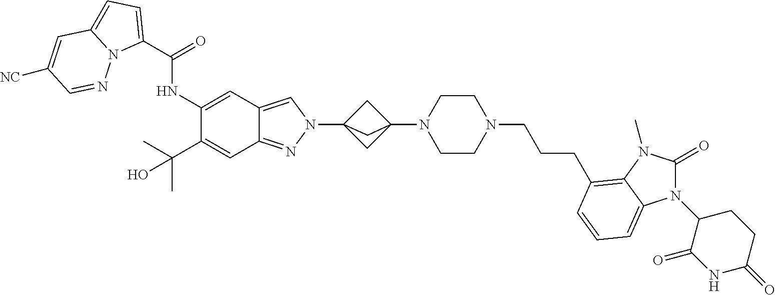 [SMILES: Cn1c(=O)n(C2CCC(=O)NC2=O)c2cccc(CCCN3CCN(C45CC(n6cc7cc(NC(=O)c8ccc9cc(C#N)cnn89)c(C(C)(C)O)cc7n6)(C4)C5)CC3)c21]

The title compound (25 mg, 31.5%) was prepared in a manner similar to that described in Example 290. ¹H NMR (500 MHz, DMSO) δ 12.05 (s, 1H), 11.09 (s, 1H), 8.93 (d, J=1.6 Hz, 1H), 8.73 (d, J=1.6 Hz, 1H), 8.59 (s, 1H), 8.35 (s, 1H), 7.72 (d, J=4.7 Hz, 1H), 7.61 (s, 1H), 7.09 (d, J=4.7 Hz, 1H), 6.97 (d, J=4.2 Hz, 2H), 6.92-6.83 (m, 1H), 5.77 (d, J=15.3 Hz, 1H), 5.37 (dd, J=12.4, 5.1 Hz, 1H), 3.58 (s, 3H), 3.02-2.83 (m, 4H), 2.76-2.55 (m, 3H), 2.47-2.35 (m, 9H), 2.33 (s, 6H), 2.05-1.95 (m, 1H), 1.80-1.72 (m, 2H), 1.63 (s, 6H). [M+H]⁺=810.7.

### Example 353: 3-cyano-N-(2-((1r,4r)-4-(4-((1-(2-(2,6-dioxopiperidin-3-yl)-1-oxoisoindolin-5-yl)piperidin-4-yl)methyl)piperazin-1-yl)cyclohexyl)-6-(2-hydroxypropan-2-yl)-2H-indazol-5-yl)pyrrolo[1,2-b]pyridazine-7-carboxamide

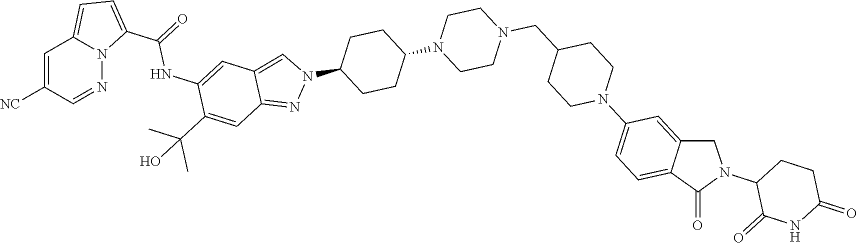 [SMILES: CC(C)(O)c1cc2nn([C@H]3CC[C@H](N4CCN(CC5CCN(c6ccc7c(c6)CN(C6CCC(=O)NC6=O)C7=O)CC5)CC4)CC3)cc2cc1NC(=O)c1ccc2cc(C#N)cnn12]

The title compound (30 mg, 45.6%) was prepared in a manner similar to that described in Example 290. ¹H NMR (500 MHz, DMSO) δ 12.02 (s, 1H), 10.94 (s, 1H), 8.93 (d, J=2.2 Hz, 1H), 8.74 (d, J=2.1 Hz, 1H), 8.55 (s, 1H), 8.33 (s, 1H), 8.17 (s, 1H), 7.72 (d, J=4.8 Hz, 1H), 7.57 (s, 1H), 7.50 (d, J=8.5 Hz, 1H), 7.09 (d, J=4.8 Hz, 1H), 7.04 (d, J=8.5 Hz, 2H), 5.72 (s, 1H), 5.04 (dd, J=13.3, 5.1 Hz, 1H), 4.42 (t, J=11.2 Hz, 1H), 4.32 (d, J=16.8 Hz, 1H), 4.19 (d, J=16.9 Hz, 1H), 3.87 (d, J=12.8 Hz, 2H), 2.98-2.76 (m, 3H), 2.68-2.52 (m, 6H), 2.46-2.27 (m, 5H), 2.22-2.09 (m, 4H), 2.04-1.87 (m, 5H), 1.78 (d, J=11.5 Hz, 3H), 1.63 (s, 6H), 1.54-1.40 (m, 2H), 1.19 (t, J=18.6 Hz, 2H). [M+H]⁺=866.8.

### Example 354: 3-cyano-N-(2-(3-(4-((1-(4-(2,4-dioxotetrahydropyrimidin-1(2H)-yl)-3,5-difluorophenyl)azetidin-3-yl)methyl)piperazin-1-yl)bicyclo[1.1.1]pentan-1-yl)-6-(2-hydroxypropan-2-yl)-2H-indazol-5-yl)pyrrolo[1,2-b]pyridazine-7-carboxamide

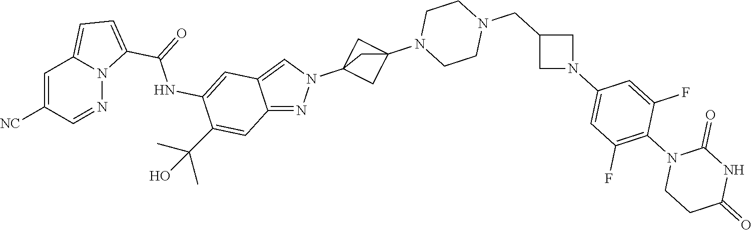 [SMILES: CC(C)(O)c1cc2nn(C34CC(N5CCN(CC6CN(c7cc(F)c(N8CCC(=O)NC8=O)c(F)c7)C6)CC5)(C3)C4)cc2cc1NC(=O)c1ccc2cc(C#N)cnn12]

The title compound (35 mg, 52.3%) was prepared in a manner similar to that described in Example 290. ¹H NMR (500 MHz, DMSO) δ 12.06 (s, 1H), 10.49 (s, 1H), 8.94 (d, J=2.1 Hz, 1H), 8.74 (d, J=2.1 Hz, 1H), 8.59 (s, 1H), 8.36 (s, 1H), 7.72 (d, J=4.7 Hz, 1H), 7.61 (s, 1H), 7.10 (d, J=4.8 Hz, 1H), 6.19 (d, J=10.2 Hz, 2H), 5.79 (s, 1H), 3.95 (t, J=7.7 Hz, 2H), 3.59 (t, J=6.7 Hz, 2H), 3.52 (dd, J=14.2, 8.2 Hz, 2H), 3.30-3.22 (m, 2H), 2.94 (dd, J=13.4, 6.9 Hz, 1H), 2.68 (t, J=6.7 Hz, 2H), 2.59 (d, J=7.3 Hz, 2H), 2.48-2.39 (m, 6H), 2.33 (s, 6H), 1.64 (s, 6H). [M+H]⁺=804.3.

### Example 398: 3-cyano-N-(2-((1r,4r)-4-(4-(1-(6-(2,6-dioxopiperidin-3-yl)-2-methylpyridin-3-yl)piperidin-4-yl)piperazin-1-yl)cyclohexyl)-6-(2-hydroxypropan-2-yl)-2H-indazol-5-yl)pyrrolo[1,2-b]pyridazine-7-carboxamide

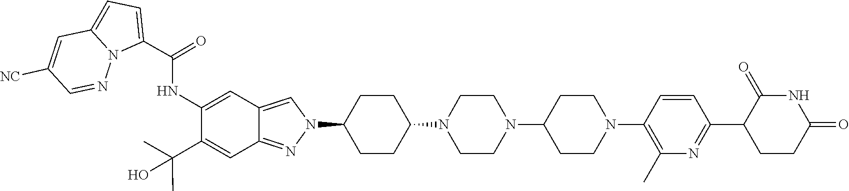 [SMILES: Cc1nc(C2CCC(=O)NC2=O)ccc1N1CCC(N2CCN([C@H]3CC[C@H](n4cc5cc(NC(=O)c6ccc7cc(C#N)cnn67)c(C(C)(C)O)cc5n4)CC3)CC2)CC1]

The title compound (68 mg, 67.8%) was prepared in a manner similar to that described in Example 290. ¹H NMR (500 MHz, DMSO) δ 11.96 (s, 1H), 10.71 (s, 1H), 8.86 (d, J=2.0 Hz, 1H), 8.67 (d, J=2.0 Hz, 1H), 8.49 (s, 1H), 8.26 (s, 1H), 7.65 (d, J=4.8 Hz, 1H), 7.51 (s, 1H), 7.29 (d, J=8.2 Hz, 1H), 7.11-6.93 (m, 2H), 5.67 (brs, 1H), 4.35 (t, J=11.5 Hz, 1H), 3.82 (dd, J=9.4, 5.3 Hz, 1H), 3.04 (d, J=10.3 Hz, 2H), 2.54-2.49 (m, 6H), 2.47 (s, 7H), 2.33 (s, 3H), 2.26 (t, J=10.9 Hz, 1H), 2.17-2.06 (m, 3H), 2.05-1.97 (m, 1H), 1.94-1.77 (m, 6H), 1.57 (s, 6H), 1.54-1.45 (m, 2H), 1.45-1.35 (m, 2H). [M+H]⁺=812.5.

### Example 399: 3-cyano-N-(2-((1R,4r)-4-((R)-4-(1-(4-((R)-2,6-dioxopiperidin-3-yl)-3,5-difluorophenyl)piperidin-4-yl)-2-(hydroxymethyl)piperazin-1-yl)cyclohexyl)-6-(2-hydroxypropan-2-yl)-2H-indazol-5-yl)pyrrolo[1,2-b]pyridazine-7-carboxamide

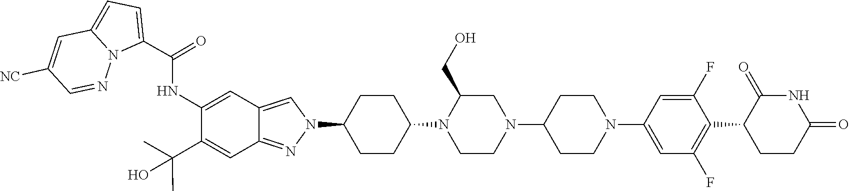 [SMILES: CC(C)(O)c1cc2nn([C@H]3CC[C@H](N4CCN(C5CCN(c6cc(F)c([C@H]7CCC(=O)NC7=O)c(F)c6)CC5)C[C@@H]4CO)CC3)cc2cc1NC(=O)c1ccc2cc(C#N)cnn12]

The title compound (1.39 mg, 7.8%) was prepared in a manner similar to that described in Example 290. ¹H NMR (500 MHz, DMSO) δ 11.95 (s, 1H), 10.80 (s, 1H), 8.87 (d, J=2.0 Hz, 1H), 8.67 (d, J=1.9 Hz, 1H), 8.48 (s, 1H), 8.27 (s, 1H), 8.07 (s, OH), 7.65 (d, J=4.8 Hz, 1H), 7.49 (s, 1H), 7.02 (d, J=4.8 Hz, 1H), 6.58 (d, J=13.2 Hz, 2H), 5.66 (s, 1H), 4.43-4.32 (m, 1H), 4.02-3.93 (m, 1H), 3.77-3.68 (m, 2H), 3.58-3.52 (m, 1H), 2.75-2.65 (m, 7H), 2.57 (s, 4H), 1.95-1.75 (m, 13H), 1.56 (s, 7H), 1.46-1.29 (m, 4H). [M+H]⁺=863.3.

### Example 400: 3-cyano-N-(2-((1R,4r)-4-((R)-4-((1-(4-((R)-2,6-dioxopiperidin-3-yl)-3,5-difluorophenyl)azetidin-3-yl)methyl)-2-(hydroxymethyl)piperazin-1-yl)cyclohexyl)-6-(2-hydroxypropan-2-yl)-2H-indazol-5-yl)pyrrolo[1,2-b]pyridazine-7-carboxamide

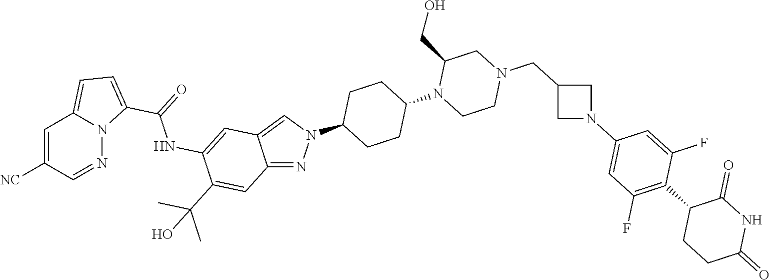 [SMILES: CC(C)(O)c1cc2nn([C@H]3CC[C@H](N4CCN(CC5CN(c6cc(F)c([C@H]7CCC(=O)NC7=O)c(F)c6)C5)C[C@@H]4CO)CC3)cc2cc1NC(=O)c1ccc2cc(C#N)cnn12]

The title compound (18 mg, 32.6%) was prepared in a manner similar to that described in Example 290. ¹H NMR (500 MHz, DMSO) δ 12.02 (s, 1H), 10.85 (s, 1H), 8.94 (s, 1H), 8.74 (s, 1H), 8.55 (s, 1H), 8.34 (s, 1H), 7.72 (d, J=4.7 Hz, 1H), 7.57 (s, 1H), 7.10 (d, J=4.7 Hz, 1H), 6.11 (d, J=11.3 Hz, 2H), 5.73 (s, 1H), 4.43 (t, J=7.5 Hz, 1H), 4.03 (dd, J=12.4, 4.5 Hz, 1H), 3.94 (t, J=7.3 Hz, 2H), 3.61 (d, J=7.7 Hz, 2H), 3.52-3.45 (m, 5H), 3.07-2.96 (m, 1H), 2.95-2.87 (m, 2H), 2.86-2.68 (m, 8H), 2.18 (d, J=9.8 Hz, 3H), 2.12-2.02 (m, 3H), 1.98-1.89 (m, 2H), 1.81 (brs, 1H), 1.63 (s, 6H), 1.50-1.40 (m, 1H). [M+H]⁺=849.2.

### Example 401: 3-cyano-N-(2-(3-(4-((1-(2-(2,6-dioxopiperidin-3-yl)-1-oxoisoindolin-5-yl)piperidin-4-yl)methyl)piperazin-1-yl)bicyclo[1.1.1]pentan-1-yl)-6-(2-hydroxypropan-2-yl)-2H-indazol-5-yl)pyrrolo[1,2-b]pyridazine-7-carboxamide

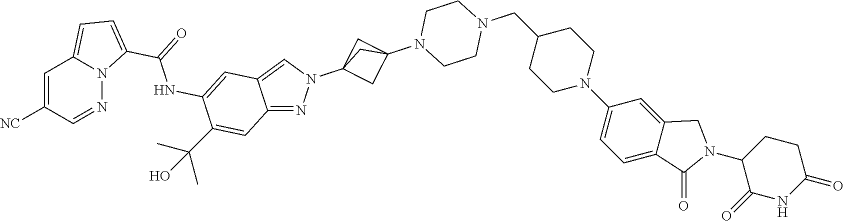 [SMILES: CC(C)(O)c1cc2nn(C34CC(N5CCN(CC6CCN(c7ccc8c(c7)CN(C7CCC(=O)NC7=O)C8=O)CC6)CC5)(C3)C4)cc2cc1NC(=O)c1ccc2cc(C#N)cnn12]

The title compound (19 mg, 33.7%) was prepared in a manner similar to that described in Example 290. ¹H NMR (500 MHz, DMSO) δ 11.99 (s, 1H), 10.87 (s, 1H), 8.87 (d, J=2.1 Hz, 1H), 8.67 (d, J=2.1 Hz, 1H), 8.53 (d, J=8.6 Hz, 1H), 8.29 (s, 1H), 7.65 (d, J=4.7 Hz, 1H), 7.53 (s, 1H), 7.44 (d, J=8.4 Hz, 1H), 7.03 (d, J=4.8 Hz, 1H), 6.98 (d, J=8.4 Hz, 2H), 5.72 (s, 1H), 4.97 (dd, J=13.3, 5.1 Hz, 1H), 4.25 (d, J=16.9 Hz, 1H), 4.13 (d, J=16.9 Hz, 1H), 3.81 (d, J=12.0 Hz, 2H), 2.89-2.71 (m, 4H), 2.59-2.46 (m, 9H), 2.33-2.19 (m, 10H), 1.96-1.82 (m, 1H), 1.72 (d, J=11.2 Hz, 3H), 1.56 (s, 6H), 1.17-1.06 (m, 2H). [M+H]⁺=850.9

### Example 109: 3-cyano-N-(2-((1r,4r)-4-(4-(1-(4-(2,4-dioxotetrahydropyrimidin-1(2H)-yl)-3,5-difluorophenyl)azetidin-3-yl)piperazin-1-yl)cyclohexyl)-6-(2-hydroxypropan-2-yl)-2H-indazol-5-yl)pyrrolo[1,2-b]pyridazine-7-carboxamide

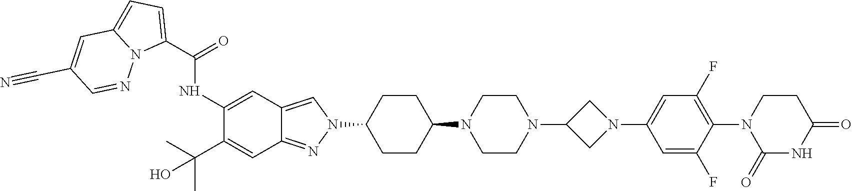 [SMILES: CC(C)(O)c1cc2nn([C@H]3CC[C@H](N4CCN(C5CN(c6cc(F)c(N7CCC(=O)NC7=O)c(F)c6)C5)CC4)CC3)cc2cc1NC(=O)c1ccc2cc(C#N)cnn12]

The title compound (16 mg, 29.7%) was prepared in a manner similar to that described in Example 290. ¹H NMR (500 MHz, DMSO) δ 12.03 (s, 1H), 10.50 (s, 1H), 8.94 (d, J=2.0 Hz, 1H), 8.74 (d, J=2.0 Hz, 1H), 8.56 (s, 1H), 8.34 (s, 1H), 7.72 (d, J=4.8 Hz, 1H), 7.57 (s, 1H), 7.09 (d, J=4.8 Hz, 1H), 6.22 (d, J=9.2 Hz, 2H), 5.73 (s, 1H), 4.63-4.38 (m, 1H), 4.05-3.87 (m, 2H), 3.72-3.51 (m, 5H), 3.47-3.35 (m, 2H), 3.19-2.96 (m, 2H), 2.67 (dd, J=18.7, 12.0 Hz, 4H), 2.45-2.11 (m, 6H), 2.07-1.88 (m, 3H), 1.77-1.61 (m, 7H), 1.56-1.40 (m, 1H). [M+H]⁺=806.6.

### Example 135: 3-cyano-N-(2-((1r,4r)-4-(4-(2-((4-(2,6-dioxopiperidin-3-yl)-3,5-difluorophenyl)amino)ethyl)piperazin-1-yl)cyclohexyl)-6-(2-hydroxypropan-2-yl)-2H-indazol-5-yl)pyrrolo[1,2-b]pyridazine-7-carboxamide

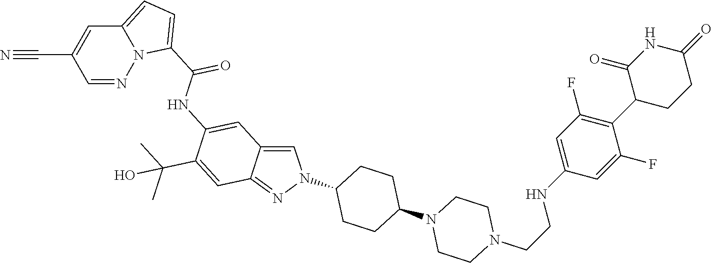 [SMILES: CC(C)(O)c1cc2nn([C@H]3CC[C@H](N4CCN(CCNc5cc(F)c(C6CCC(=O)NC6=O)c(F)c5)CC4)CC3)cc2cc1NC(=O)c1ccc2cc(C#N)cnn12]

The title compound (52 mg, 46%) was prepared in a manner similar to that described in Example 290. ¹H NMR (500 MHz, DMSO) δ 12.02 (s, 1H), 10.83 (s, 1H), 8.93 (d, J=2.0 Hz, 1H), 8.74 (d, J=2.0 Hz, 1H), 8.55 (s, 1H), 8.33 (s, 1H), 7.72 (d, J=4.8 Hz, 1H), 7.57 (s, 1H), 7.09 (d, J=4.8 Hz, 1H), 6.27 (d, J=12.0 Hz, 2H), 6.07 (s, 1H), 5.72 (s, 1H), 4.43 (t, J=12.0 Hz, 1H), 3.98 (dd, J=12.4, 5.0 Hz, 1H), 3.19-3.08 (m, 3H), 2.79-2.74 (m, 1H), 2.70-2.58 (m, 6H), 2.40-2.25 (m, 3H), 2.22-2.16 (m, 2H), 2.10-1.91 (m, 7H), 1.63 (s, 6H), 1.55-1.47 (m, 2H), 1.25-1.20 (m, 1H). [M+H]⁺=793.6.

### Example 198: (R)-3-cyano-N-(2-(1′-(1-(4-(2,4-dioxotetrahydropyrimidin-1(2H)-yl)-3,5-difluorophenyl)azetidine-3-carbonyl)-3′,3′-difluoro-[1,4′-bipiperidin]-4-yl)-6-(2-hydroxypropan-2-yl)-2H-indazol-5-yl)pyrrolo[1,2-b]pyridazine-7-carboxamide

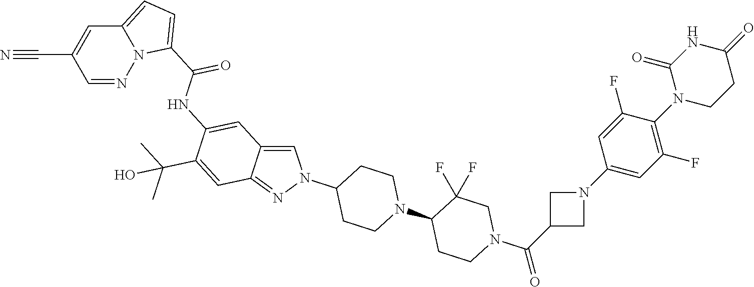 [SMILES: CC(C)(O)c1cc2nn(C3CCN([C@@H]4CCN(C(=O)C5CN(c6cc(F)c(N7CCC(=O)NC7=O)c(F)c6)C5)CC4(F)F)CC3)cc2cc1NC(=O)c1ccc2cc(C#N)cnn12]

The title compound (50 mg, 76.26%) was prepare in a manner similar to at described in Example 178. ¹H NMR (500 MHz, DMSO) δ 12.03 (s, 1H), 10.50 (s, 1H), 8.93 (d, J=2.1 Hz, 1H), 8.74 (d, J=2.1 Hz, 1H), 8.56 (s, 1H), 8.38 (s, 1H), 7.72 (d, J=4.8 Hz, 1H), 7.58 (s, 1H), 7.09 (d, J=4.8 Hz, 1H), 6.27 (t, J=10.2 Hz, 2H), 5.73 (s, 1H), 4.63-4.41 (m, 2H), 4.13-4.04 (m, 2H), 3.95-3.70 (m, 3H), 3.88-3.72 (m, 1H), 3.59 (t, J=6.7 Hz, 2H), 3.50-3.35 (m, 1H), 3.26-3.03 (m, 4H), 3.01-2.72 (m, 3H), 2.68 (t, J=6.7 Hz, 2H), 2.17-2.00 (m, 4H), 1.90-1.80 (m, 1H), 1.63 (s, 6H). [M+H]⁺=870.6.

### Example 414: 3-bromo-N-(2-((1R,4r)-4-(4-(4-((R)-2,6-dioxopiperidin-3-yl)-3,5-difluorophenethyl)piperazin-1-yl)cyclohexyl)-6-(2-hydroxypropan-2-yl)-2H-indazol-5-yl)pyrrolo[1,2-b]pyridazine-7-carboxamide

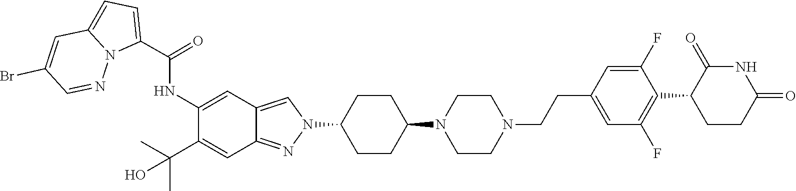 [SMILES: CC(C)(O)c1cc2nn([C@H]3CC[C@H](N4CCN(CCc5cc(F)c([C@H]6CCC(=O)NC6=O)c(F)c5)CC4)CC3)cc2cc1NC(=O)c1ccc2cc(Br)cnn12]

The title compound (19.2 mg, 33.93%) was prepared in a manner similar to that described in Example 290. ¹H NMR (500 MHz, DMSO) δ 11.91 (s, 1H), 10.95 (s, 1H), 8.58 (dd, J=12.8, 2.4 Hz, 2H), 8.53 (s, 1H), 8.31 (s, 1H), 7.59-7.54 (m, 2H), 7.02 (d, J=10.1 Hz, 2H), 6.73 (d, J=4.6 Hz, 1H), 5.66 (s, 1H), 4.42 (t, J=11.4 Hz, 1H), 4.20 (dd, J=12.5, 5.0 Hz, 1H), 2.85-2.72 (m, 3H), 2.60-2.53 (m, 7H), 2.45-2.35 (m, 5H), 2.18-2.11 (m, 3H), 2.02-1.90 (m, 5H), 1.63 (s, 6H), 1.52-1.45 (m, 2H). [M+H]⁺=831.6.

### Example 383: 3-cyano-N-(2-((1R,4r)-4-(4-(((1S,3S)-3-(4-((R)-2,6-dioxopiperidin-3-yl)-3,5-difluorophenyl)cyclobutyl)methyl)piperazin-1-yl)cyclohexyl)-6-(2-hydroxypropan-2-yl)-2H-indazol-5-yl)pyrrolo[1,2-b]pyridazine-7-carboxamide

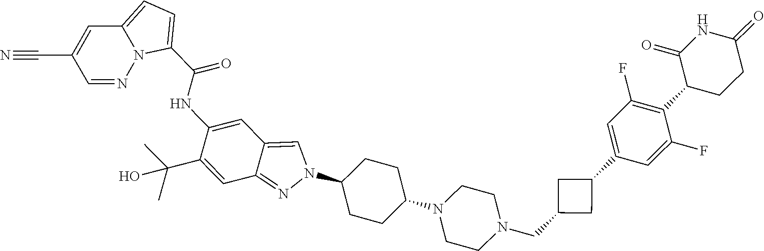 [SMILES: CC(C)(O)c1cc2nn([C@H]3CC[C@H](N4CCN(C[C@H]5C[C@@H](c6cc(F)c([C@H]7CCC(=O)NC7=O)c(F)c6)C5)CC4)CC3)cc2cc1NC(=O)c1ccc2cc(C#N)cnn12]

The title compound (39 mg, 27.20%) was prepared in a manner similar to that described in Example 290. ¹H NMR (500 MHz, DMSO) δ 12.02 (s, 1H), 10.95 (s, 1H), 8.93 (d, J=2.0 Hz, 1H), 8.74 (d, J=2.0 Hz, 1H), 8.55 (s, 1H), 8.33 (s, 1H), 8.16 (s, 1H), 7.72 (d, J=4.8 Hz, 1H), 7.57 (s, 1H), 7.30 (dd, J=15.3, 8.5 Hz, 1H), 7.07 (dd, J=18.4, 6.9 Hz, 2H), 5.72 (s, 1H), 4.42 (t, J=11.2 Hz, 1H), 4.22 (dd, J=12.6, 5.0 Hz, 1H), 3.57-3.45 (m, 2H), 2.85-2.78 (m, 1H), 2.60-2.53 (m, 5H), 2.47-2.35 (m, 9H), 2.20-2.08 (m, 3H), 2.03-1.89 (m, 5H), 1.78-1.73 (m, 2H), 1.63 (s, 6H), 1.52-1.41 (m, 2H). [M+H]⁺=818.6.

### Example 384: 3-cyano-N-(2-((1r,4r)-4-(4-(1-(4-((R)-2,6-dioxopiperidin-3-yl)-3,5-difluorophenyl)azetidine-3-carbonyl)piperazin-1-yl)cyclohexyl)-7,7-difluoro-2H-[1,3]dioxolo[4,5-g]indazol-5-yl)pyrrolo[1,2-b]pyridazine-7-carboxamide

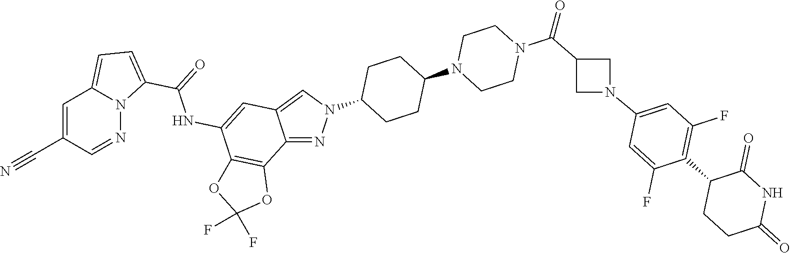 [SMILES: N#Cc1cnn2c(C(=O)Nc3cc4cn([C@H]5CC[C@H](N6CCN(C(=O)C7CN(c8cc(F)c([C@H]9CCC(=O)NC9=O)c(F)c8)C7)CC6)CC5)nc4c4c3OC(F)(F)O4)ccc2c1]

The title compound (33.5 mg, 47.33%) was prepared in a manner similar to that described in Example 290. ¹H NMR (500 MHz, DMSO) δ 10.86 (s, 1H), 10.84 (s, 1H), 9.01 (d, J=2.1 Hz, 1H), 8.97 (d, J=2.0 Hz, 1H), 8.68 (s, 1H), 8.27 (s, 1H), 7.79 (d, J=4.8 Hz, 1H), 7.13 (d, J=4.8 Hz, 1H), 6.17 (d, J=11.1 Hz, 2H), 4.51 (t, J=11.6 Hz, 1H), 4.08-4.01 (m, 3H), 3.92 (t, J=6.2 Hz, 2H), 3.86-3.79 (m, 1H), 3.55-3.45 (m, 2H), 3.31-3.27 (m, 2H), 2.82-2.72 (m, 1H), 2.57-2.50 (m, 6H), 2.25-2.18 (m, 2H), 2.13-2.03 (m, 1H), 1.02-1.90 (m, 5H), 1.55-1.45 (m, 2H). [M+H]⁺=855.6.

### Example 385: 3-cyano-N-(2-((1R,4r)-4-(4-(1-(4-((R)-2,6-dioxopiperidin-3-yl)-3,5-difluorophenyl)azetidin-3-yl)piperazin-1-yl)cyclohexyl)-7,7-difluoro-2H-[1,3]dioxolo[4,5-g]indazol-5-yl)pyrrolo[1,2-b]pyridazine-7-carboxamide

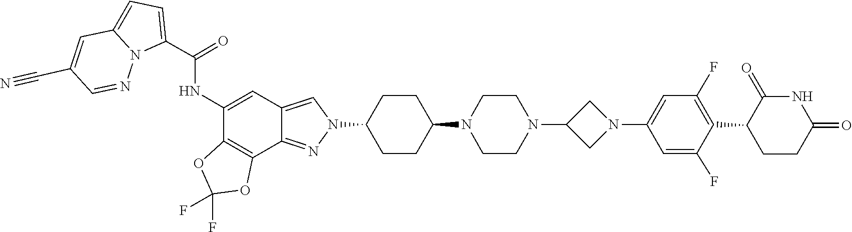 [SMILES: N#Cc1cnn2c(C(=O)Nc3cc4cn([C@H]5CC[C@H](N6CCN(C7CN(c8cc(F)c([C@H]9CCC(=O)NC9=O)c(F)c8)C7)CC6)CC5)nc4c4c3OC(F)(F)O4)ccc2c1]

The title compound (14 mg, 20.21%) was prepared in a manner similar to that described in Example 290. ¹H NMR (500 MHz, DMSO) δ 10.85 (d, J=9.6 Hz, 2H), 9.01 (d, J=2.1 Hz, 1H), 8.97 (d, J=2.1 Hz, 1H), 8.68 (s, 1H), 8.27 (s, 1H), 7.79 (d, J=4.8 Hz, 1H), 7.13 (d, J=4.8 Hz, 1H), 6.12 (d, J=11.1 Hz, 2H), 4.50 (t, J=11.6 Hz, 1H), 4.03 (dd, J=12.6, 5.0 Hz, 1H), 3.91 (t, J=7.1 Hz, 2H), 3.67-3.60 (m, 2H), 3.26-3.19 (m, 2H), 2.81-2.73 (m, 1H), 2.62-2.53 (m, 4H), 2.47-2.28 (m, 5H), 2.23-2.17 (m, 2H), 2.11-2.03 (m, 1H), 2.00-1.90 (m, 5H), 1.54-1.44 (m, 2H). [M+H]⁺=827.6.

### Example 386: 3-cyano-N-(2-((1R,4r)-4-(4-(4-((R)-2,6-dioxopiperidin-3-yl)-3,5-difluorophenethyl)piperazin-1-yl)cyclohexyl)-7,7-difluoro-2H-[1,3]dioxolo[4,5-g]indazol-5-yl)pyrrolo[1,2-b]pyridazine-7-carboxamide

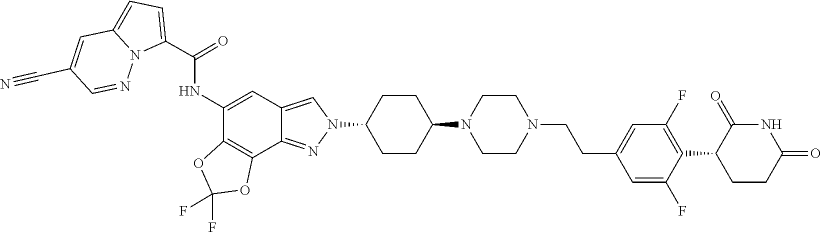 [SMILES: N#Cc1cnn2c(C(=O)Nc3cc4cn([C@H]5CC[C@H](N6CCN(CCc7cc(F)c([C@H]8CCC(=O)NC8=O)c(F)c7)CC6)CC5)nc4c4c3OC(F)(F)O4)ccc2c1]

The title compound (31 mg, 35.33%) was prepared in a manner similar to that described in Example 290. ¹H NMR (500 MHz, DMSO) δ 10.95 (s, 1H), 10.84 (s, 1H), 9.01 (d, J=2.1 Hz, 1H), 8.97 (d, J=2.1 Hz, 1H), 8.68 (s, 1H), 8.27 (s, 1H), 7.79 (d, J=4.8 Hz, 1H), 7.13 (d, J=4.8 Hz, 1H), 7.02 (d, J=10.0 Hz, 2H), 4.54-4.45 (m, 1H), 4.20 (dd, J=12.6, 4.9 Hz, 1H), 2.85-2.72 (m, 4H), 2.60-2.53 (m, 7H), 2.48-2.36 (m, 4H), 2.23-2.07 (m, 2H), 2.15-2.08 (m, 1H), 2.02-1.91 (m, 5H), 1.54-1.42 (m, 2H). [M+H]+=800.6.

### Example 395: 3-cyano-N-(2-((1S,4r)-4-(4-((3S)-1-(4-(2,6-dioxopiperidin-3-yl)-3,5-difluorophenyl)pyrrolidin-3-yl)piperazin-1-yl)cyclohexyl)-6-(2-hydroxypropan-2-yl)-2H-indazol-5-yl)pyrrolo[1,2-b]pyridazine-7-carboxamide

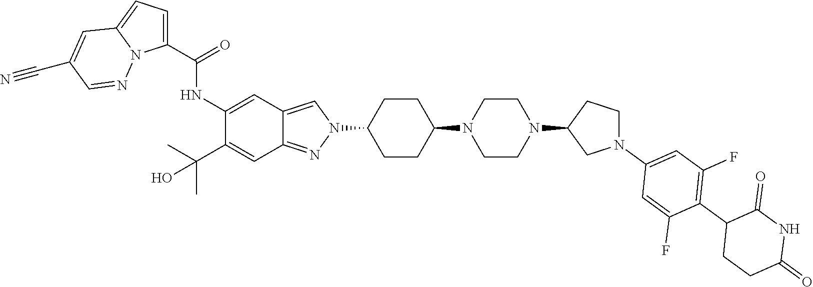 [SMILES: CC(C)(O)c1cc2nn([C@H]3CC[C@H](N4CCN([C@H]5CCN(c6cc(F)c(C7CCC(=O)NC7=O)c(F)c6)C5)CC4)CC3)cc2cc1NC(=O)c1ccc2cc(C#N)cnn12]

The title compound (9 mg, 11.18%) was prepared in a manner similar to that described in Example 290. ¹H NMR (500 MHz, DMSO) δ 12.03 (s, 1H), 10.84 (s, 1H), 8.94 (d, J=2.1 Hz, 1H), 8.74 (d, J=2.1 Hz, 1H), 8.56 (s, 1H), 8.34 (s, 1H), 7.72 (d, J=4.8 Hz, 1H), 7.57 (s, 1H), 7.47 (d, J=8.0 Hz, 1H), 7.14-7.07 (m, 2H), 6.24 (d, J=12.3 Hz, 2H), 5.72 (s, 1H), 4.52-4.42 (m, 1H), 4.02 (dd, J=12.7, 4.8 Hz, 1H), 3.52-3.45 (m, 1H), 3.42-3.35 (m, 4H), 3.24-3.19 (m, 1H), 3.10-2.63 (m, 10H), 2.29 (s, 1H), 2.25-2.18 (m, 3H), 2.11-1.93 (m, 6H), 1.87-1.80 (m, 1H), 1.63 (s, 6H), 1.61-1.50 (m, 2H). [M+H]⁺=819.6.

### Example 397: 3-cyano-N-(2-((1r,4r)-4-(4-((1-(4-((R)-2,6-dioxopiperidin-3-yl)-3,5-difluorophenyl)azetidin-3-yl)methyl)piperazin-1-yl)cyclohexyl)-6-(2-hydroxypropan-2-yl)-2H-indazol-5-yl)-1,2-dihydropyrrolo[1,2-b]pyridazine-7-carboxamide

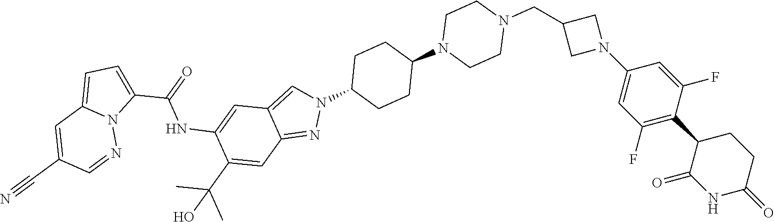 [SMILES: CC(C)(O)c1cc2nn([C@H]3CC[C@H](N4CCN(CC5CN(c6cc(F)c([C@H]7CCC(=O)NC7=O)c(F)c6)C5)CC4)CC3)cc2cc1NC(=O)c1ccc2cc(C#N)cnn12]

The title compound (140 mg, 5.26%) was prepared in a manner similar to that described in Example 178. ¹H NMR (500 MHz, DMSO) δ 11.59 (s, 1H), 10.85 (s, 1H), 8.41 (s, 1H), 8.29 (s, 1H), 7.59 (s, 1H), 7.54 (s, 1H), 6.81-6.72 (m, 2H), 6.43 (d, J=4.2 Hz, 1H), 6.10 (d, J=11.2 Hz, 2H), 5.64 (s, 1H), 4.40 (s, 1H), 4.02 (dd, J=12.6, 4.8 Hz, 1H), 3.96-3.88 (m, 4H), 3.47 (t, J=5.8 Hz, 2H), 2.94-2.89 (m, 1H), 2.82-2.74 (m, 1H), 2.57-2.53 (m, 8H), 2.45-2.35 (m, 4H), 2.20-2.13 (m, 2H), 2.10-2.02 (m, 1H), 1.97-1.87 (m, 5H), 1.60 (s, 6H), 1.51-1.42 (m, 2H). [M+H]⁺=821.6.

### Example 402: 3-cyano-N-(2-((1R,4r)-4-((3R)-4-((1-(2-(2,6-dioxopiperidin-3-yl)-1-oxoisoindolin-5-yl)piperidin-4-yl)methyl)-3-(methoxymethyl)piperazin-1-yl)cyclohexyl)-6-(2-hydroxypropan-2-yl)-2H-indazol-5-yl)pyrrolo[1,2-b]pyridazine-7-carboxamide

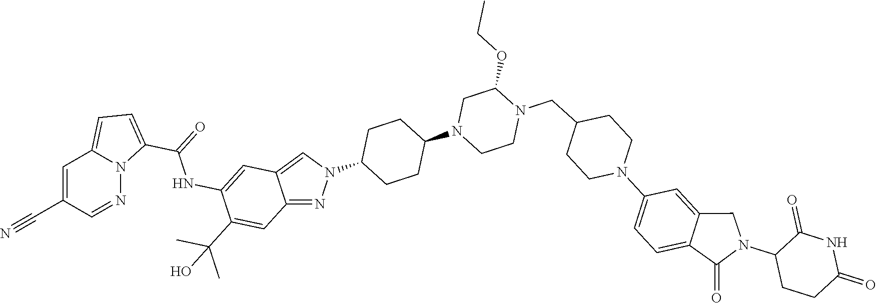 [SMILES: CCO[C@H]1CN([C@H]2CC[C@H](n3cc4cc(NC(=O)c5ccc6cc(C#N)cnn56)c(C(C)(C)O)cc4n3)CC2)CCN1CC1CCN(c2ccc3c(c2)CN(C2CCC(=O)NC2=O)C3=O)CC1]

The title compound (77 mg, 58.28%) was prepared in a manner similar to that described in Example 290. ¹H NMR (500 MHz, DMSO) δ 11.95 (s, 1H), 10.87 (s, 1H), 8.86 (d, J=2.1 Hz, 1H), 8.67 (d, J=2.1 Hz, 1H), 8.48 (s, 1H), 8.26 (s, 1H), 8.07 (s, OH), 7.65 (d, J=4.8 Hz, 1H), 7.51 (s, 1H), 7.43 (d, J=8.5 Hz, 1H), 7.02 (d, J=4.8 Hz, 1H), 6.97 (d, J=8.3 Hz, 2H), 5.65 (d, J=6.9 Hz, 1H), 4.97 (dd, J=13.3, 5.0 Hz, 1H), 4.36 (t, J=11.3 Hz, 1H), 4.25 (d, J=16.8 Hz, 1H), 4.13 (d, J=16.8 Hz, 1H), 3.80 (d, J=11.8 Hz, 2H), 3.42 (dd, J=9.5, 3.8 Hz, 1H), 3.17 (s, 3H), 2.90-2.50 (m, 10H), 2.35-2.09 (m, 7H), 2.05-1.99 (m, 1H), 1.93-1.84 (m, 5H), 1.76 (d, J=12.0 Hz, 1H), 1.65-1.53 (m, 8H), 1.47-1.35 (m, 2H), 1.16-1.02 (m, 2H). [M+H]⁺=910.6.

### Example 411: 6-cyano-N-(2-((1r,4r)-4-(4-(1-(4-((R)-2,6-dioxopiperidin-3-yl)-3,5-difluorophenyl)azetidine-3-carbonyl)piperazin-1-yl)cyclohexyl)-6-(2-hydroxypropan-2-yl)-2H-indazol-5-yl)pyrazolo[1,5-a]pyrimidine-3-carboxamide

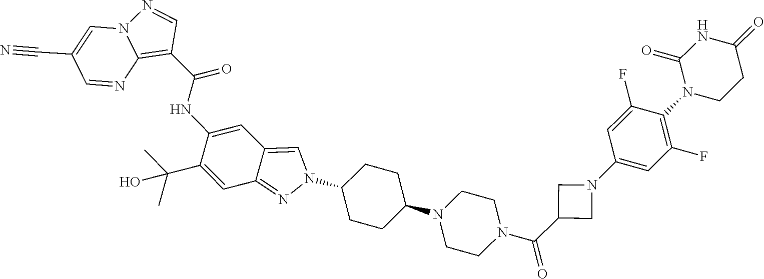

The title compound (2.03 mg, 9%) was prepared in a manner similar to that described in Example 178. ¹H NMR (500 MHz, DMSO) δ 10.79 (s, 1H), 9.90 (s, 1H), 9.07 (s, 1H), 8.68 (s, 1H), 8.60 (s, 1H), 8.30 (d, J=31.5 Hz, 2H), 7.49 (s, 1H), 7.35 (s, 1H), 6.10 (d, J=11.3 Hz, 2H), 4.41-4.33 (m, 1H), 4.01-3.92 (m, 3H), 3.88-3.82 (m, 2H), 3.80-3.73 (m, 1H), 3.48-3.35 (m, 6H), 2.75-2.66 (m, 1H), 2.52-2.45 (m, 4H), 2.15-2.08 (m, 2H), 2.05-1.96 (m, 1H), 1.93-1.83 (m, 5H), 1.65 (s, 6H), 1.48-1.38 (m, 2H). [M+H]⁺=834.6.

### Example 344: 2-((1r,4r)-4-(4-(1-(4-((R)-2,6-dioxopiperidin-3-yl)-3,5-difluorophenyl)azetidine-3-carbonyl)piperazin-1-yl)cyclohexyl)-6-methoxy-N-(pyrazolo[1,5-a]pyrimidin-3-yl)-2H-indazole-5-carboxamide

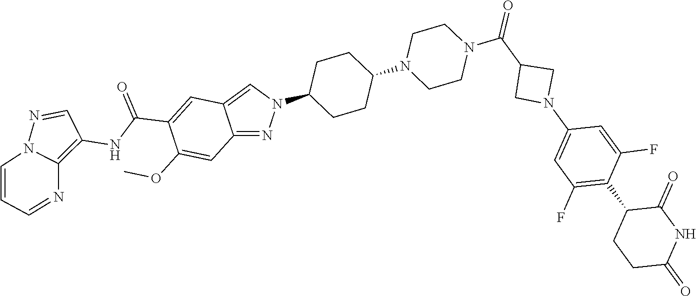 [SMILES: COc1cc2nn([C@H]3CC[C@H](N4CCN(C(=O)C5CN(c6cc(F)c([C@H]7CCC(=O)NC7=O)c(F)c6)C5)CC4)CC3)cc2cc1C(=O)Nc1cnn2cccnc12]

The title compound was prepared in a manner similar to that described in Example 178. 1H NMR (500 MHz, DMSO) δ 10.84 (s, 1H), 10.34 (s, 1H), 9.07 (dd, J=7.1, 1.5 Hz, 1H), 8.73 (s, 1H), 8.52 (dd, J=21.1, 16.4 Hz, 3H), 7.21 (s, 1H), 7.05 (dd, J=7.0, 4.0 Hz, 1H), 6.16 (d, J=11.1 Hz, 2H), 4.49-4.40 (m, 1H), 4.11-3.99 (m, 6H), 3.95-3.77 (m, 3H), 3.52-3.46 (m, 2H), 2.83-2.72 (m, 1H), 2.58-2.51 (m, 5H), 2.49-2.42 (m, 3H), 2.24-2.16 (m, 2H), 2.13-2.01 (m, 1H), 2.00-1.87 (m, 5H), 1.59-1.43 (m, 2H). [M+H]⁺=781.6.

### Example 415: 3-cyano-N-(2-((1R,4r)-4-((2R)-4-(1-(6-(2,6-dioxopiperidin-3-yl)-2-methylpyridin-3-yl)piperidin-4-yl)-2-(methoxymethyl)piperazin-1-yl)cyclohexyl)-6-(2-hydroxypropan-2-yl)-2H-indazol-5-yl)pyrrolo[1,2-b]pyridazine-7-carboxamide

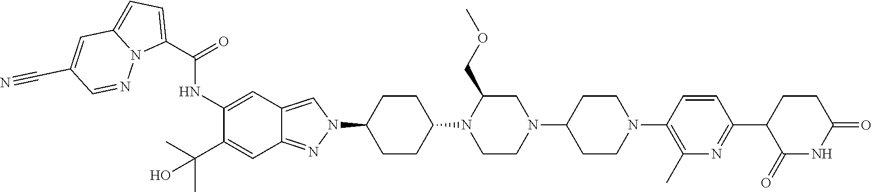 [SMILES: COC[C@H]1CN(C2CCN(c3ccc(C4CCC(=O)NC4=O)nc3C)CC2)CCN1[C@H]1CC[C@H](n2cc3cc(NC(=O)c4ccc5cc(C#N)cnn45)c(C(C)(C)O)cc3n2)CC1]

The title compound (50 mg, 29.2%) was prepared in a manner similar to that described in Example 290. ¹H NMR (500 MHz, DMSO) δ 12.03 (s, 1H), 10.78 (s, 1H), 8.94 (d, J=2.0 Hz, 1H), 8.74 (d, J=2.0 Hz, 1H), 8.56 (s, 1H), 8.34 (s, 1H), 7.72 (d, J=5.0 Hz, 1H), 7.58 (s, 1H), 7.36 (d, J=8.0 Hz, 1H), 7.13-7.08 (m, 2H), 5.77-5.68 (m, 1H), 4.46-4.38 (m, 1H), 3.92-3.87 (m, 1H), 3.56-3.51 (m, 1H), 3.42-3.35 (m, 2H), 3.30-3.29 (m, 3H), 3.29-3.28 (m, 3H), 3.11 (d, J=10.0 Hz, 2H), 2.87-2.80 (m, 2H), 2.79-2.73 (m, 2H), 2.66-2.61 (m, 3H), 2.59-2.56 (m, 3H), 2.39 (s, 3H), 2.28-2.21 (m, 2H), 2.19-2.15 (m, 2H), 2.10-2.06 (m, 1H), 2.03-1.96 (m, 2H), 1.89-1.83 (m, 2H), 1.80-1.75 (m, 1H), 1.64 (s, 6H), 1.61-1.56 (m, 2H), 1.45-1.38 (m, 1H). [M+H]⁺=856.8

### Example 417: 3-cyano-N-(2-((1r,4r)-4-(4-(1-(2-(2,6-dioxopiperidin-3-yl)-1-oxoisoindolin-5-yl)piperidin-4-yl)piperazin-1-yl)cyclohexyl)-6-(2-hydroxypropan-2-yl)-2H-indazol-5-yl)pyrrolo[1,2-b]pyridazine-7-carboxamide

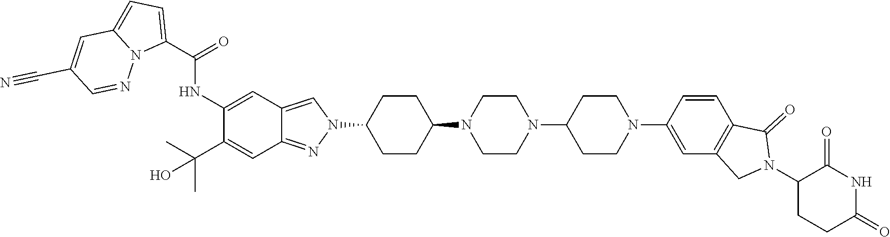 [SMILES: CC(C)(O)c1cc2nn([C@H]3CC[C@H](N4CCN(C5CCN(c6ccc7c(c6)CN(C6CCC(=O)NC6=O)C7=O)CC5)CC4)CC3)cc2cc1NC(=O)c1ccc2cc(C#N)cnn12]

The title compound (36 mg, 21.1%) was prepared in a manner similar to that described in Example 290. ¹H NMR (500 MHz, DMSO) δ 12.02 (s, 1H), 10.93 (s, 1H), 8.94 (d, J=2.0 Hz, 1H), 8.74 (d, J=2.0 Hz, 1H), 8.54 (s, 1H), 8.33 (s, 1H), 7.71 (d, J=4.5 Hz, 1H), 7.57 (s, 1H), 7.50 (d, J=8.5 Hz, 1H), 7.14-6.92 (m, 3H), 5.71 (s, 1H), 5.04 (dd, J=13.0, 5.0 Hz, 1H), 4.47-4.38 (m, 1H), 4.35-4.28 (m, 1H), 4.23-4.17 (m, 1H), 3.96-3.84 (m, 2H), 2.93-2.79 (m, 3H), 2.65-2.51 (m, 8H), 2.41-2.32 (m, 4H), 2.21-2.14 (m, 2H), 2.00-1.91 (m, 5H), 1.88-1.80 (m, 2H), 1.63 (s, 6H), 1.54-1.41 (m, 4H). [M+H]⁺=852.6.

### Example 416: 3-cyano-N-(2-(4-(4-(1-(6-(2,6-dioxopiperidin-3-yl)-2-methylpyridin-3-yl)piperidin-4-yl)piperazin-1-yl)bicyclo[2.2.2]octan-1-yl)-6-(2-hydroxypropan-2-yl)-2H-indazol-5-yl)pyrrolo[1,2-b]pyridazine-7-carboxamide

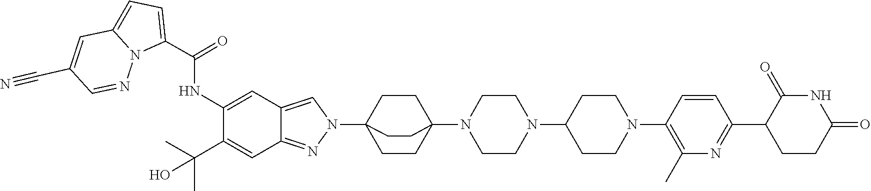 [SMILES: Cc1nc(C2CCC(=O)NC2=O)ccc1N1CCC(N2CCN(C34CCC(n5cc6cc(NC(=O)c7ccc8cc(C#N)cnn78)c(C(C)(C)O)cc6n5)(CC3)CC4)CC2)CC1]

The title compound (83 mg, 32.24%) was prepared in a manner similar to that described in Example 290. ¹H NMR (500 MHz, DMSO) δ 12.03 (s, 1H), 10.87 (s, 1H), 8.93 (d, J=2.0 Hz, 1H), 8.74 (d, J=2.0 Hz, 1H), 8.56 (s, 1H), 8.33 (s, 1H), 7.72 (d, J=4.8 Hz, 1H), 7.57 (s, 1H), 7.09 (d, J=4.8 Hz, 1H), 6.62 (d, J=12.8 Hz, 2H), 5.71 (s, 1H), 4.04 (dd, J=12.6, 4.9 Hz, 1H), 3.76 (d, J=11.9 Hz, 2H), 2.82-2.70 (m, 3H), 2.50-2.46 (m, 11H), 2.32-2.28 (m, 1H), 2.21-2.15 (m, 6H), 2.12-2.04 (m, 1H), 1.99-1.93 (m, 1H), 1.82-1.72 (m, 9H), 1.63 (s, 6H), 1.40 (dd, J=20.9, 10.3 Hz, 2H). [M+H]⁺=838.6.

### Example 419: 3-cyano-N-(2-(3-(4-(1-(6-(2,6-dioxopiperidin-3-yl)-2-methylpyridin-3-yl)piperidin-4-yl)piperazin-1-yl)bicyclo[1.1.1]pentan-1-yl)-6-(2-hydroxypropan-2-yl)-2H-indazol-5-yl)pyrrolo[1,2-b]pyridazine-7-carboxamide

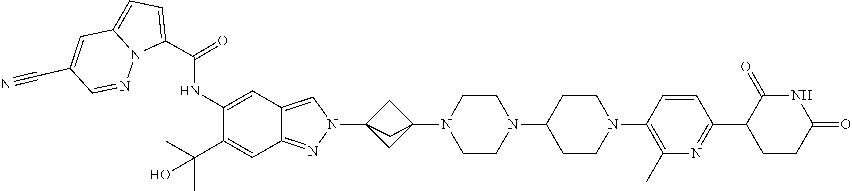 [SMILES: Cc1nc(C2CCC(=O)NC2=O)ccc1N1CCC(N2CCN(C34CC(n5cc6cc(NC(=O)c7ccc8cc(C#N)cnn78)c(C(C)(C)O)cc6n5)(C3)C4)CC2)CC1]

The title compound (278 mg, 62.21%) was prepared in a manner similar to that described in Example 290. ¹H NMR (500 MHz, DMSO) δ 12.06 (s, 1H), 10.78 (s, 1H), 8.93 (d, J=1.8 Hz, 1H), 8.74 (d, J=1.8 Hz, 1H), 8.60 (s, 1H), 8.35 (s, 1H), 7.72 (d, J=4.7 Hz, 1H), 7.61 (s, 1H), 7.36 (d, J=8.2 Hz, 1H), 7.13-7.05 (m, 2H), 5.78 (s, 1H), 3.89 (dd, J=9.3, 5.3 Hz, 1H), 3.14-3.08 (m, 2H), 2.65-2.54 (m, 8H), 2.50-2.46 (m, 3H), 2.39 (s, 3H), 2.36-2.30 (m, 7H), 2.23-2.17 (m, 1H), 2.11-2.04 (m, 1H), 1.90-1.83 (m, 2H), 1.66-1.55 (m, 9H). [M+H]⁺=796.6.

### Example 426: 3-cyano-N-(2-((1r,4r)-4-(4-(1-(6-(2,6-dioxopiperidin-3-yl)-2-methylpyridin-3-yl)azetidin-3-yl)piperazin-1-yl)cyclohexyl)-6-(2-hydroxypropan-2-yl)-2H-indazol-5-yl)pyrrolo[1,2-b]pyridazine-7-carboxamide

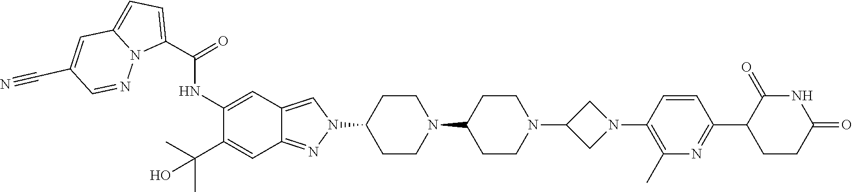

The title compound (23 mg, 20.63%) was prepared in a manner similar to that described in Example 290. ¹H NMR (500 MHz, DMSO) δ 12.02 (s, 1H), 10.74 (s, 1H), 8.93 (d, J=2.0 Hz, 1H), 8.74 (d, J=2.0 Hz, 1H), 8.55 (s, 1H), 8.33 (s, 1H), 7.72 (d, J=4.8 Hz, 1H), 7.57 (s, 1H), 7.09 (d, J=4.8 Hz, 1H), 7.01 (d, J=8.2 Hz, 1H), 6.81 (d, J=8.3 Hz, 1H), 5.72 (s, 1H), 4.42 (t, J=11.3 Hz, 1H), 4.03-3.93 (m, 2H), 3.82 (dd, J=9.0, 5.3 Hz, 1H), 3.64 (t, J=6.5 Hz, 2H), 3.17 (dt, J=12.6, 6.4 Hz, 1H), 2.64-2.52 (m, 7H), 2.46-2.34 (m, 4H), 2.31 (s, 3H), 2.22-2.14 (m, 3H), 2.09-2.03 (m, 1H), 1.98-1.88 (m, 4H), 1.63 (s, 6H), 1.53-1.43 (m, 2H). [M+H]⁺=784.6.

### Example 433: N-(2-((1r,4r)-4-((4-(1-(4-((R)-2,6-dioxopiperidin-3-yl)-3,5-difluorophenyl)azetidin-3-yl)piperazin-1-yl)methyl)cyclohexyl)-6-(2-hydroxypropan-2-yl)-2H-indazol-5-yl)-2-(1H-pyrazol-4-yl)thiazole-4-carboxamide

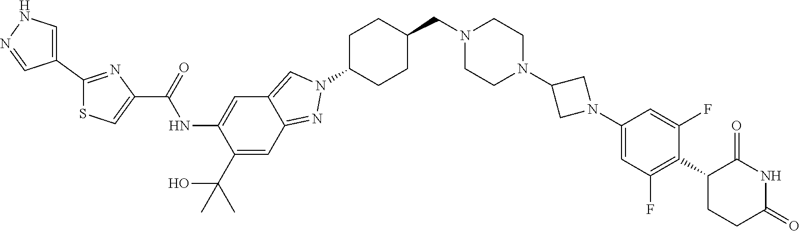 [SMILES: CC(C)(O)c1cc2nn([C@H]3CC[C@H](CN4CCN(C5CN(c6cc(F)c([C@H]7CCC(=O)NC7=O)c(F)c6)C5)CC4)CC3)cc2cc1NC(=O)c1csc(-c2cn[nH]c2)n1]

The title compound (22 mg, 23.21%) was prepared in a manner similar to that described in Example 290. ¹H NMR (500 MHz, DMSO) δ 13.37 (s, 1H), 12.08 (s, 1H), 10.86 (s, 1H), 8.70 (s, 1H), 8.57-8.15 (m, 4H), 7.56 (s, 1H), 6.25-5.94 (m, 3H), 4.40 (t, J=11.5 Hz, 1H), 4.03 (dd, J=12.4, 4.9 Hz, 1H), 3.90 (t, J=7.0 Hz, 2H), 3.66-3.60 (m, 2H), 3.27-3.23 (m, 2H), 2.83-2.74 (m, 1H), 2.48-2.30 (m, 9H), 2.16-2.04 (m, 5H), 1.96-1.86 (m, 5H), 1.67 (s, 6H), 1.15-1.04 (m, 2H). [M+H]⁺=827.6.

### Example 434: N-(2-((1r,4r)-4-((4-(1-(4-((R)-2,6-dioxopiperidin-3-yl)-3,5-difluorophenyl)azetidine-3-carbonyl)piperazin-1-yl)methyl)cyclohexyl)-6-(2-hydroxypropan-2-yl)-2H-indazol-5-yl)-2-(1H-pyrazol-4-yl)thiazole-4-carboxamide

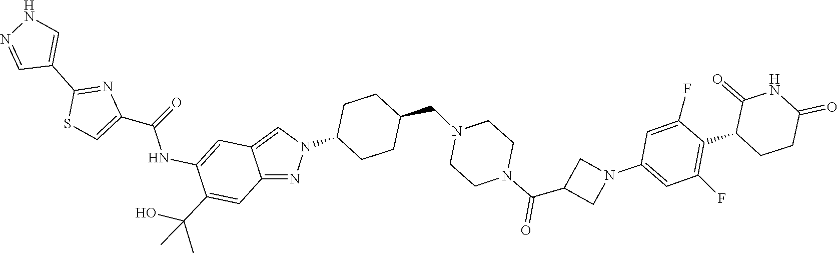 [SMILES: CC(C)(O)c1cc2nn([C@H]3CC[C@H](CN4CCN(C(=O)C5CN(c6cc(F)c([C@H]7CCC(=O)NC7=O)c(F)c6)C5)CC4)CC3)cc2cc1NC(=O)c1csc(-c2cn[nH]c2)n1]

The title compound (23 mg, 20.28%) was prepared in a manner similar to that described in Example 178. ¹H NMR (500 MHz, DMSO) δ 13.38 (s, 1H), 12.08 (s, 1H), 10.86 (s, 1H), 8.70 (s, 1H), 8.47 (s, 1H), 8.33 (s, 1H), 8.23 (s, 1H), 8.16 (s, 1H), 7.57 (s, 1H), 6.17 (d, J=11.1 Hz, 2H), 6.04 (s, 1H), 4.48-4.38 (m, 1H), 4.07-4.00 (m, 3H), 3.94-3.89 (m, 2H), 3.86-3.80 (m, 1H), 3.52-3.47 (m, 2H), 2.82-2.73 (m, 1H), 2.56-2.51 (m, 4H), 2.40-2.30 (m, 4H), 2.22-2.04 (m, 5H), 1.98-1.86 (m, 5H), 1.67 (s, 6H), 1.17-1.08 (m, 2H). [M+H]⁺=855.6.

### Example 431: 3-cyano-N-(2-((1S,4r)-4-(4-(((3S)-1-(2-(2,6-dioxopiperidin-3-yl)-1,3-dioxoisoindolin-5-yl)pyrrolidin-3-yl)methyl)piperazin-1-yl)cyclohexyl)-6-(2-hydroxypropan-2-yl)-2H-indazol-5-yl)pyrrolo[1,2-b]pyridazine-7-carboxamide

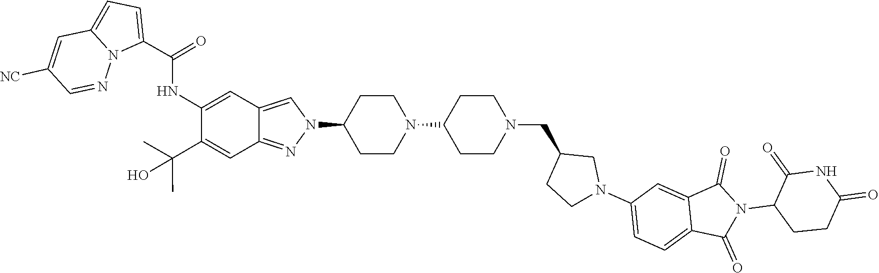

The title compound (40 mg, 48.60%) was prepared in a manner similar to that described in Example 290. ¹H NMR (500 MHz, DMSO)¹H NMR (500 MHz, DMSO) δ 12.02 (s, 1H), 11.07 (s, 1H), 8.93 (d, J=2.0 Hz, 1H), 8.74 (d, J=2.0 Hz, 1H), 8.55 (s, 1H), 8.33 (s, 1H), 7.72 (d, J=4.7 Hz, 1H), 7.64 (d, J=8.4 Hz, 1H), 7.57 (s, 1H), 7.09 (d, J=4.8 Hz, 1H), 6.89 (s, 1H), 6.81 (d, J=8.6 Hz, 1H), 5.72 (brs, 1H), 5.05 (dd, J=12.8, 5.3 Hz, 1H), 4.42 (t, J=11.5 Hz, 1H), 3.58-3.46 (m, 3H), 3.39-3.33 (m, 2H), 3.15-3.10 (m, 1H), 2.93-2.83 (m, 1H), 2.65-2.53 (m, 6H), 2.48-2.28 (m, 6H), 2.22-2.07 (m, 3H), 2.06-1.87 (m, 5H), 1.75 (td, J=15.5, 7.6 Hz, 1H), 1.63 (s, 6H), 1.56-1.41 (m, 2H). [M+H]⁺=866.4.

### Example 432: 3-cyano-N-(2-((1R,4r)-4-(4-(((3R)-1-(2-(2,6-dioxopiperidin-3-yl)-1,3-dioxoisoindolin-5-yl)pyrrolidin-3-yl)methyl)piperazin-1-yl)cyclohexyl)-6-(2-hydroxypropan-2-yl)-2H-indazol-5-yl)pyrrolo[1,2-b]pyridazine-7-carboxamide

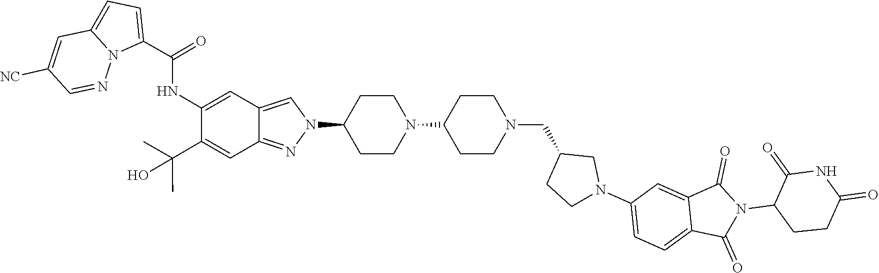

The title compound (35 mg, 42.52%) was prepared in a manner similar to that described in Example 290. ¹H NMR (500 MHz, DMSO)¹H NMR (500 MHz, DMSO) δ 12.03 (s, 1H), 11.07 (s, 1H), 8.92 (d, J=1.9 Hz, 1H), 8.74 (d, J=2.0 Hz, 1H), 8.54 (d, J=21.3 Hz, 1H), 8.33 (s, 1H), 7.72 (d, J=4.7 Hz, 1H), 7.63 (d, J=8.4 Hz, 1H), 7.57 (s, 1H), 7.09 (d, J=4.7 Hz, 1H), 6.88 (s, 1H), 6.80 (d, J=8.5 Hz, 1H), 5.74 (brs, 1H), 5.06 (dd, J=12.9, 5.3 Hz, 1H), 4.42 (t, J=11.4 Hz, 1H), 3.52 (dd, J=21.0, 12.9 Hz, 2H), 3.38 (dd, J=17.3, 7.7 Hz, 1H), 3.18-3.08 (m, 1H), 2.96-2.82 (m, 1H), 2.70-2.53 (m, 8H), 2.48-2.26 (m, 6H), 2.24-2.07 (m, 3H), 2.05-1.86 (m, 5H), 1.74 (td, J=15.6, 7.6 Hz, 1H), 1.64 (s, 6H), 1.55-1.41 (m, 2H). [M+H]+=866.4.

### Example 418: 3-cyano-N-(2-((1S,4r)-4-((3S)-4-(1-(6-(2,6-dioxopiperidin-3-yl)-2-methylpyridin-3-yl)piperidin-4-yl)-3-(methoxymethyl)piperazin-1-yl)cyclohexyl)-6-(2-hydroxypropan-2-yl)-2H-indazol-5-yl)pyrrolo[1,2-b]pyridazine-7-carboxamide

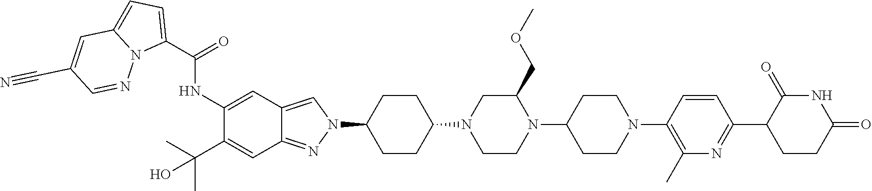 [SMILES: COC[C@@H]1CN([C@H]2CC[C@H](n3cc4cc(NC(=O)c5ccc6cc(C#N)cnn56)c(C(C)(C)O)cc4n3)CC2)CCN1C1CCN(c2ccc(C3CCC(=O)NC3=O)nc2C)CC1]

The title compound (13.7 mg, 49.3%) was prepared in a manner similar to that described in Example 290. ¹H NMR (500 MHz, DMSO) δ 12.02 (s, 1H), 10.78 (s, 1H), 8.93 (d, J=5 Hz, 1H), 8.74 (d, J=5 Hz, 1H), 8.55 (s, 1H), 8.33 (s, 1H), 7.72 (d, J=5 Hz, 1H), 7.58 (s, 1H), 7.35 (d, J=5 Hz, 1H), 7.11-7.08 (m, 2H), 5.71 (s, 1H), 4.46-4.39 (m, 1H), 3.88 (dd, J=5 Hz, 10 Hz, 1H), 3.55-3.51 (m, 1H), 3.40-3.35 (m, 1H), 3.10 (s, 3H), 3.14-3.07 (m, 2H), 2.87-2.83 (m, 1H), 2.81-2.53 (m, 9H), 2.44-2.33 (m, 6H), 2.25-2.14 (m, 3H), 2.11-2.04 (m, 1H), 1.99-1.84 (m, 5H), 1.76-1.67 (m, 2H), 1.63 (s, 6H), 1.58-1.41 (m, 3H). [M+H]⁺=856.6

### Example 425: N-(6-acetyl-2-((1R,4r)-4-(4-(1-(4-((R)-2,6-dioxopiperidin-3-yl)-3,5-difluorophenyl)azetidin-3-yl)piperazin-1-yl)cyclohexyl)-2H-indazol-5-yl)-3-cyanopyrrolo[1,2-b]pyridazine-7-carboxamide

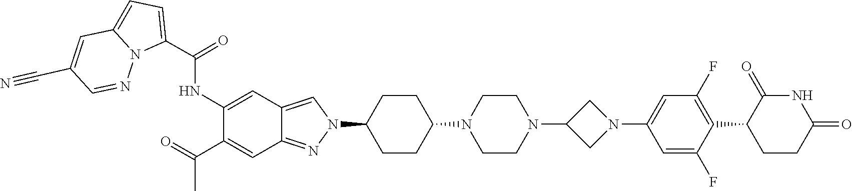 [SMILES: CC(=O)c1cc2nn([C@H]3CC[C@H](N4CCN(C5CN(c6cc(F)c([C@H]7CCC(=O)NC7=O)c(F)c6)C5)CC4)CC3)cc2cc1NC(=O)c1ccc2cc(C#N)cnn12]

The title compound (131.08 mg, 57.64%) was prepared in a manner similar to that described in Example 290. ¹H NMR (500 MHz, DMSO) δ 12.29 (s, 1H), 10.86 (s, 1H), 8.95 (d, J=5 Hz, 1H), 8.85 (d, J=5 Hz, 1H), 8.79 (s, 1H), 8.51 (d, J=10 Hz, 2H), 7.73 (d, J=5 Hz, 1H), 7.09 (d, J=5 Hz, 1H), 6.12 (d, J=10 Hz, 2H), 4.55-4.47 (m, 1H), 4.03 (dd, J=5 Hz, 10 Hz, 1H), 3.90 (t, J=10 Hz, 2H), 3.66-3.60 (m, 2H), 3.27-3.21 (m, 1H), 2.82-2.73 (m, 4H), 2.65-2.53 (m, 5H), 2.48-2.27 (m, 5H), 2.24-2.17 (m, 2H), 2.11-2.03 (m, 1H), 2.01-1.90 (m, 5H), 1.54-1.44 (m, 2H). [M+H]⁺=789.6

### Example 427: 3-cyano-N-(2-(3-(1-(6-(2,6-dioxopiperidin-3-yl)-2-methylpyridin-3-yl)piperidin-4-yl)-3-azaspiro[5.5]undecan-9-yl)-6-(2-hydroxypropan-2-yl)-2H-indazol-5-yl)pyrrolo[1,2-b]pyridazine-7-carboxamide

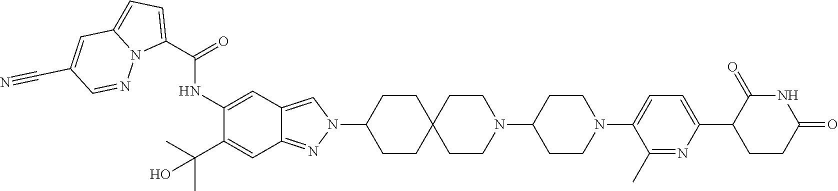 [SMILES: Cc1nc(C2CCC(=O)NC2=O)ccc1N1CCC(N2CCC3(CCC(n4cc5cc(NC(=O)c6ccc7cc(C#N)cnn67)c(C(C)(C)O)cc5n4)CC3)CC2)CC1]

The title compound (21.03 mg, 23%) was prepared in a manner similar to that described in Example 290. ¹H NMR (500 MHz, DMSO) δ 12.02 (s, 1H), 10.78 (s, 1H), 8.93 (d, J=5 Hz, 1H), 8.74 (d, J=5 Hz, 1H), 8.55 (s, 1H), 8.38 (s, 1H), 7.72 (d, J=5 Hz, 1H), 7.59 (s, 1H), 7.36 (d, J=5 Hz, 1H), 7.13-7.06 (m, 2H), 5.73 (s, 1H), 4.46-4.39 (m, 1H), 3.88 (dd, J=5 Hz, 10 Hz, 1H), 3.12 (d, J=10 Hz, 2H), 2.65-2.52 (m, 8H), 2.45-2.35 (m, 4H), 2.25-2.15 (m, 1H), 2.12-2.03 (m, 3H), 1.96-1.77 (m, 6H), 1.70-1.57 (m, 10H), 1.45-1.38 (m, 2H), 1.35-1.26 (m, 2H). [M+H]⁺=797.7

### Example 428: (R)-3-cyano-N-(2-(3-(1-(4-(2,6-dioxopiperidin-3-yl)-3,5-difluorophenyl)azetidin-3-yl)-3-azaspiro[5.5]undecan-9-yl)-6-(2-hydroxypropan-2-yl)-2H-indazol-5-yl)pyrrolo[1,2-b]pyridazine-7-carboxamide

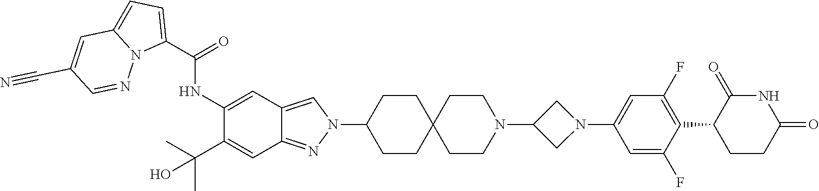 [SMILES: CC(C)(O)c1cc2nn(C3CCC4(CC3)CCN(C3CN(c5cc(F)c([C@H]6CCC(=O)NC6=O)c(F)c5)C3)CC4)cc2cc1NC(=O)c1ccc2cc(C#N)cnn12]

The title compound (55.51 mg, 35.9%) was prepared in a manner similar to that described in Example 290. ¹H NMR (500 MHz, DMSO) δ 12.02 (s, 1H), 10.86 (s, 1H), 8.93 (d, J=5 Hz, 1H), 8.74 (d, J=5 Hz, 1H), 8.55 (s, 1H), 8.38 (s, 1H), 7.72 (d, J=5 Hz, 1H), 7.58 (s, 1H), 7.09 (d, J=5 Hz, 1H), 6.12 (d, J=10 Hz, 2H), 5.72 (s, 1H), 4.47-4.38 (m, 1H), 4.03 (dd, J=5 Hz, 10 Hz, 1H), 3.92 (t, J=10 Hz, 2H), 3.64-3.59 (m, 2H), 3.31-3.24 (m, 2H), 2.82-2.73 (m, 1H), 2.35-2.27 (m, 4H), 2.12-2.01 (m, 3H), 1.98-1.89 (m, 3H), 1.84-1.76 (m, 2H), 1.43-1.37 (m, 2H), 1.35-1.26 (m, 2H). [M+H]⁺=790.7

### Example 429: 3-cyano-N-(2-((1r,4r)-4-(4-(1-(2-(2,6-dioxopiperidin-3-yl)-1,3-dioxoisoindolin-5-yl)piperidine-4-carbonyl)piperazin-1-yl)cyclohexyl)-6-(2-hydroxypropan-2-yl)-2H-indazol-5-yl)pyrrolo[1,2-b]pyridazine-7-carboxamide

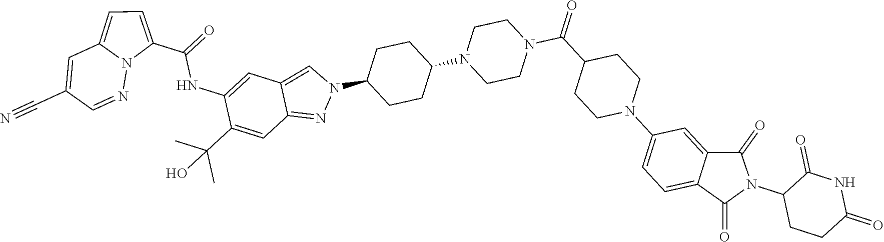 [SMILES: CC(C)(O)c1cc2nn([C@H]3CC[C@H](N4CCN(C(=O)C5CCN(c6ccc7c(c6)C(=O)N(C6CCC(=O)NC6=O)C7=O)CC5)CC4)CC3)cc2cc1NC(=O)c1ccc2cc(C#N)cnn12]

The title compound (239.18 mg, 70.4%) was prepared in a manner similar to that described in Example 178. ¹H NMR (500 MHz, DMSO) δ 12.02 (s, 1H), 11.07 (s, 1H), 8.93 (d, J=5 Hz, 1H), 8.74 (d, J=5 Hz, 1H), 8.55 (s, 1H), 8.34 (s, 1H), 8.14 (s, 1H), 7.71 (d, J=5 Hz, 1H), 7.66 (d, J=5 Hz, 1H), 7.57 (s, 1H), 7.33 (s, 1H), 7.25 (d, J=5 Hz, 1H), 7.09 (d, J=5 Hz, 1H), 5.72 (s, 1H), 5.07 (dd, J=5 Hz, 10 Hz, 1H), 4.46-4.38 (m, 1H), 4.07 (d, J=5 Hz, 2H), 3.58-3.52 (m, 2H), 3.49-3.43 (m, 2H), 3.08 (d, J=5 Hz, 2H), 3.01-2.83 (m, 2H), 2.65-2.52 (m, 5H), 2.49-2.45 (m, 2H), 2.45-2.25 (m, 5H), 2.22-2.15 (m, 2H), 2.05-1.90 (m, 5H), 1.74-1.67 (m, 2H), 1.66-1.56 (m, 8H), 1.55-1.45 (m, 2H). [M+H]⁺=894.7

### Example 430: 3-cyano-N-(2-((1r,4r)-4-(4-(1-(2-(2,6-dioxopiperidin-3-yl)-1,3-dioxoisoindolin-5-yl)piperidin-4-yl)piperazin-1-yl)cyclohexyl)-6-(2-hydroxypropan-2-yl)-2H-indazol-5-yl)pyrrolo[1,2-b]pyridazine 7-carboxamide

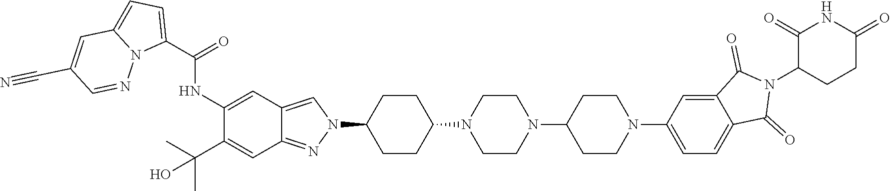 [SMILES: CC(C)(O)c1cc2nn([C@H]3CC[C@H](N4CCN(C5CCN(c6ccc7c(c6)C(=O)N(C6CCC(=O)NC6=O)C7=O)CC5)CC4)CC3)cc2cc1NC(=O)c1ccc2cc(C#N)cnn12]

The title compound (15.9 mg, 29%) was prepared in a manner similar to that described in Example 290. ¹H NMR (500 MHz, DMSO) δ 12.02 (s, 1H), 11.07 (s, 1H), 8.93 (d, J=5 Hz, 1H), 8.74 (d, J=5 Hz, 1H), 8.55 (s, 1H), 8.33 (s, 1H), 7.71 (d, J=5 Hz, 1H), 7.66 (d, J=5 Hz, 1H), 7.57 (s, 1H), 7.32 (s, 1H), 7.25 (d, J=5 Hz, 1H), 7.09 (d, J=5 Hz, 1H), 5.72 (s, 1H), 5.07 (dd, J=5 Hz, 10 Hz, 1H), 4.45-4.37 (m, 1H), 4.06 (d, J=5 Hz, 2H), 3.01-2.83 (m, 3H), 2.65-2.52 (m, 6H), 2.48-2.33 (m, 6H), 2.19-2.13 (m, 2H), 2.04-1.81 (m, 7H), 1.63 (s, 6H), 1.51-1.36 (m, 4H). [M+H]⁺=866.8

### Example 435: N-(3-(difluoromethyl)-1-((1r,4r)-4-(4-((1-(4-((R)-2,6-dioxopiperidin-3-yl)-3,5-difluorophenyl)azetidin-3-yl)methyl)piperazin-1-yl)cyclohexyl)-1H-pyrazol-4-yl)-2-(1H-pyrazol-4-yl)thiazole-4-carboxamide

### Step 1: tert-butyl 4-((1r,4r)-4-(4-(2-(1H-pyrazol-4-yl)thiazole-4-carboxamido)-3-(difluoromethyl)-1H-pyrazol-1-yl)cyclohexyl)piperazine-1-carboxylate

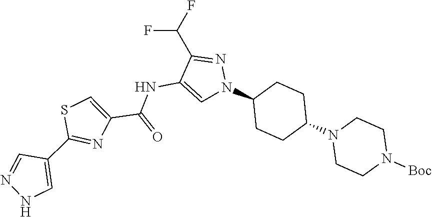 [SMILES: CC(C)(C)OC(=O)N1CCN([C@H]2CC[C@H](n3cc(NC(=O)c4csc(-c5cn[nH]c5)n4)c(C(F)F)n3)CC2)CC1]

To a solution of tert-butyl 4-((1r,4r)-4-(4-amino-3-(difluoromethyl)-1H-pyrazol-1-yl)cyclohexyl)piperazine-1-carboxylate (600 mg, 1.5 mmol) and 2-(1H-pyrazol-4-yl)thiazole-4-carboxylic acid (292.5 mg, 1.5 mmol) in DMF (10 mL) was added HATU (570 mg, 1.5 mmol) and DIEA (580.5 mg, 4.5 mmol). The mixture was stirred for 2 h at rt. The mixture was diluted with water (30 mL) and extracted with DCM (3×50 mL). The combined organic layer was washed water, brine, dried over anhydrous Na₂SO₄, filtered and concentrated under vacuum. The residue was purified on silica gel column chromatography (DCM:MeOH=15:1) to afford product (740 g, 85.65%). [M+H]⁺=577.2

### Step 2: N-(3-(difluoromethyl)-1-((1r,4r)-4-(piperazin-1-yl)cyclohexyl)-1H-pyrazol-4-yl)-2-(1H-pyrazol-4-yl)thiazole-4-carboxamide

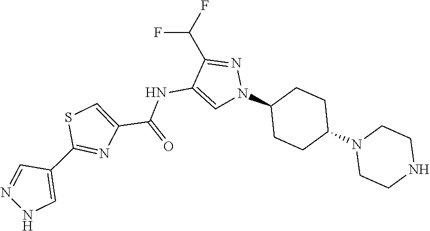 [SMILES: O=C(Nc1cn([C@H]2CC[C@H](N3CCNCC3)CC2)nc1C(F)F)c1csc(-c2cn[nH]c2)n1]

To a stirred solution of tert-butyl 4-((1r,4r)-4-(4-(2-(1H-pyrazol-4-yl)thiazole-4-carboxamido)-3-(difluoromethyl)-1H-pyrazol-1-yl)cyclohexyl)piperazine-1-carboxylate (740 mg, 1.285 mmol) in DCM (10 mL) was added TFA (2 mL). The mixture was stirred for 2 h at rt. The mixture was diluted with water (10 mL), then adjusted PH 8 with sat.NaHCO₃ solution and extracted with DCM (3×20 mL). The combined organic layer was washed water, brine, dried over anhydrous Na₂SO₄, filtered and concentrated under vacuum to afford product (520 mg, 85%). [M+H]⁺=477.2

### Step 3: N-(3-(difluoromethyl)-1-((1r,4r)-4-(4-((1-(4-((R)-2,6-dioxopiperidin-3-yl)-3,5-difluorophenyl)azetidin-3-yl)methyl)piperazin-1-yl)cyclohexyl)-1H-pyrazol-4-yl)-2-(1H-pyrazol-4-yl)thiazole-4-carboxamide

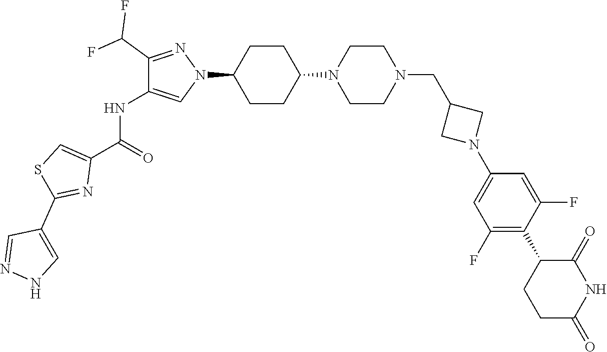 [SMILES: O=C1CC[C@H](c2c(F)cc(N3CC(CN4CCN([C@H]5CC[C@H](n6cc(NC(=O)c7csc(-c8cn[nH]c8)n7)c(C(F)F)n6)CC5)CC4)C3)cc2F)C(=O)N1]

To a solution of N-(3-(difluoromethyl)-1-((1r,4r)-4-(piperazin-1-yl)cyclohexyl)-1H-pyrazol-4-yl)-2-(1H-pyrazol-4-yl)thiazole-4-carboxamide (40 mg, 0.084 mmol) and (R)-1-(4-(2,6-dioxopiperidin-3-yl)-3,5-difluorophenyl)azetidine-3-carbaldehyde (CAS: 2873368-99-9, which was obtained in the same way described in WO2022012622A1) (51.75 mg, 0.168 mmol) in DCM (10 mL) was added NaBH(OAc)₃ (35.62 mg, 0.168 mmol). The mixture was stirred for 2 h at rt. The mixture was diluted with water (20 mL) and extracted with DCM (3×30 mL). The combined organic layers were dried over anhydrous Na₂SO₄, filtered and evaporated in vacuum. The residue was purified by Prep-HPLC (C-18 column chromatography (0.1% FA in water:acetonitrile=90:10˜60:40 gradient elution) to give the desired product (25.66 mg, 39.78%).

¹H NMR (500 MHz, DMSO) δ 13.38 (s, 1H), 10.85 (s, 1H), 9.73 (s, 1H), 8.43 (br s, 1H), 8.30 (s, 1H), 8.21 (s, 1H), 8.14 (s, 1H), 7.16 (t, J=5 Hz, 1H), 6.10 (d, J=10 Hz, 2H), 4.25-4.17 (m, 1H), 4.02 (dd, J=5 Hz, 10 Hz, 1H), 3.92 (t, J=10 Hz, 2H), 3.47 (d, J=10 Hz, 2H), 2.95-2.85 (m, 1H), 2.82-2.73 (m, 1H), 2.65-2.52 (m, 6H), 2.48-2.30 (m, 6H), 2.13-2.02 (m, 3H), 1.96-1.87 (m, 3H), 1.84-1.73 (m, 2H), 1.46-1.35 (m, 2H). [M+H]⁺=769.8

### Example 437: 3-cyano-N-(2-((1r,4r)-4-(4-(2-((2-(2,6-dioxopiperidin-3-yl)-1,3-dioxoisoindolin-4-yl)amino)ethyl)piperazin-1-yl)cyclohexyl)-6-(2-hydroxypropan-2-yl)-2H-indazol-5-yl)pyrrolo[1,2-b]pyridazine-7-carboxamide

### Step 1: 2-(2,6-dioxopiperidin-3-yl)-4-((2-hydroxyethyl)amino)isoindoline-1,3-dione

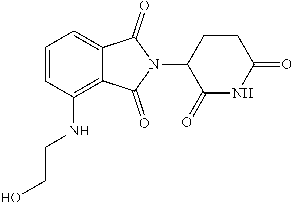 [SMILES: O=C1CCC(N2C(=O)c3cccc(NCCO)c3C2=O)C(=O)N1]

To a solution of 2-(2,6-dioxopiperidin-3-yl)-4-fluoroisoindoline-1,3-dione (2.76 g, 10 mmol) in DMSO (50 mL) was added 2-aminoethan-1-ol (1.22 g, 20 mmol) and DIEA (3.87 g, 30 mmol). The mixture was stirred overnight at 90° C. under N₂. The mixture was diluted with water (100 mL) and extracted with DCM (3×200 mL). The combined organic layer was washed water, brine, dried over anhydrous Na₂SO₄, filtered and concentrated under vacuum. The residue was purified on silica gel column chromatography (DCM:MeOH=15:1) to afford product (1.28 g, 40.38%). [M+H]⁺=318.1

### Step 2: 2-((2-(2,6-dioxopiperidin-3-yl)-1,3-dioxoisoindolin-4-yl)amino)ethyl methanesulfonate

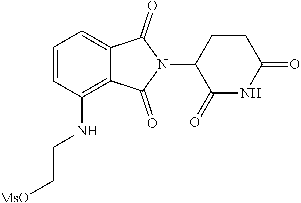 [SMILES: CS(=O)(=O)OCCNc1cccc2c1C(=O)N(C1CCC(=O)NC1=O)C2=O]

To a stirred solution of 2-(2,6-dioxopiperidin-3-yl)-4-((2-hydroxyethyl)amino)isoindoline-1,3-dione (1.28 g, 4.025 mmol) and Et₃N (1.22 g, 12.075 mmol) in DCM (30 mL) was added methanesulfonyl chloride (0.55 g, 4.83 mmol) at 0° C. The mixture was stirred for 2 h at 0° C. The mixture was diluted with water (30 mL) and extracted with DCM (3×50 mL). The combined organic layer was washed water, brine, dried over anhydrous Na₂SO₄, filtered and concentrated under vacuum to afford product (1.3 g, 81.56%). [M+H]⁺=396.1

### Step 3: 3-cyano-N-(2-((1r,4r)-4-(4-(2-((2-(2,6-dioxopiperidin-3-yl)-1,3-dioxoisoindolin-4-yl)amino)ethyl)piperazin-1-yl)cyclohexyl)-6-(2-hydroxypropan-2-yl)-2H-indazol-5-yl)pyrrolo[1,2-b]pyridazine-7-carboxamide

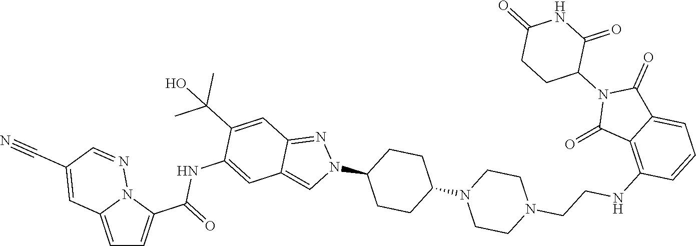 [SMILES: CC(C)(O)c1cc2nn([C@H]3CC[C@H](N4CCN(CCNc5cccc6c5C(=O)N(C5CCC(=O)NC5=O)C6=O)CC4)CC3)cc2cc1NC(=O)c1ccc2cc(C#N)cnn12]

To a solution of 3-cyano-N-(6-(2-hydroxypropan-2-yl)-2-((1r,4r)-4-(piperazin-1-yl)cyclohexyl)-2H-indazol-5-yl)pyrrolo[1,2-b]pyridazine-7-carboxamide (263 mg, 0.5 mmol) and 2-((2-(2,6-dioxopiperidin-3-yl)-1,3-dioxoisoindolin-4-yl)amino)ethyl methanesulfonate (297 mg, 0.75 mmol) in acetonitrile (10 mL) and DMSO (5 mL) was added DIEA (193.5 mg, 1.5 mmol) and KI (166 mg, 1 mmol). The resulting mixture was stirred overnight at 80° C. under N₂. The mixture was diluted with water (30 mL) and extracted with DCM (3×50 mL). The combined organic layers were dried over anhydrous Na₂SO₄, filtered and evaporated in vacuum. The residue was purified by Prep-HPLC (C-18 column chromatography (0.1% FA in water:acetonitrile=90:10˜60:40 gradient elution) to give the desired product (187.02 mg, 45.28%).

¹H NMR (500 MHz, DMSO) δ 12.02 (s, 1H), 11.09 (s, 1H), 8.93 (d, J=5.0 Hz, 1H), 8.74 (d, J=5.0 Hz, 1H), 8.55 (s, 1H), 8.33 (s, 1H), 7.72 (d, J=5 Hz, 1H), 7.62-7.52 (m, 2H), 7.10 (t, J=10 Hz, 2H), 7.04 (d, J=5 Hz, 1H), 6.75 (s, 1H), 5.72 (s, 1H), 5.08 (dd, J=5 Hz, 10 Hz, 1H), 4.46-4.38 (m, 1H), 3.39-3.35 (m, 2H), 2.93-2.84 (m, 1H), 2.65-2.52 (m, 11H), 2.48-2.35 (m, 2H), 2.21-2.15 (m, 2H), 2.06-1.90 (m, 5H), 1.63 (s, 6H), 1.54-1.43 (m, 2H). [M+H]⁺=826.63

### Example 439: 3-cyano-N-(2-((1S,4r)-4-(4-(2-(((2S)-1-((2,6-dioxopiperidin-3-yl)amino)-1-oxo-3-phenylpropan-2-yl)amino)-2-oxoethyl)piperazin-1-yl)cyclohexyl)-6-(2-hydroxypropan-2-yl)-2H-indazol-5-yl)pyrrolo[1,2-b]pyridazine-7-carboxamide

### Step 1: tert-butyl ((2S)-1-((2,6-dioxopiperidin-3-yl)amino)-1-oxo-3-phenylpropan-2-yl)carbamate

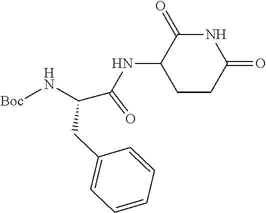 [SMILES: CC(C)(C)OC(=O)N[C@@H](Cc1ccccc1)C(=O)NC1CCC(=O)NC1=O]

To a solution of (tert-butoxycarbonyl)-L-phenylalanine (2.65 g, 10 mmol) and 3-aminopiperidine-2,6-dione (1.536 g, 12 mmol) in DCM (50 mL) was added EDCI (2.872 g, 15 mmol) and HOBT (2.04 g, 15 mmol). The mixture was stirred overnight at rt. The mixture was diluted with water (50 mL) and extracted with DCM (3×50 mL). The combined organic layer was washed water, brine, dried over anhydrous Na₂SO₄, filtered and concentrated under vacuum to afford product (2.2 g, 58.67%). [M+H]⁺=376.2

### Step 2: (2S)-2-amino-N-(2,6-dioxopiperidin-3-yl)-3-phenylpropanamide

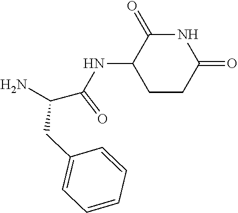 [SMILES: N[C@@H](Cc1ccccc1)C(=O)NC1CCC(=O)NC1=O]

To a stirred solution of tert-butyl ((2S)-1-((2,6-dioxopiperidin-3-yl)amino)-1-oxo-3-phenylpropan-2-yl)carbamate (2.2 g, 5.87 mmol) in DCM (40 mL) was added HCl/1,4-dioxane (4 N, 10 mL). The mixture was stirred for 2 h at rt. The mixture was concentrated under vacuum to afford product (1.8 g crude). [M+H]⁺=276.1

### Step 3: (2S)-2-(2-bromoacetamido)-N-(2,6-dioxopiperidin-3-yl)-3-phenylpropanamide

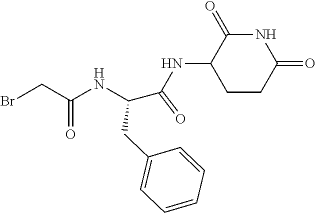 [SMILES: O=C1CCC(NC(=O)[C@H](Cc2ccccc2)NC(=O)CBr)C(=O)N1]

To a solution of (2S)-2-amino-N-(2,6-dioxopiperidin-3-yl)-3-phenylpropanamide hydrochloride (1.8 g crude, 5.87 mmol) in DCM (50 mL) was added Et₃N (1.778 g, 17.61 mmol) and 2-bromoacetyl bromide (1.409 g, 7.044 mmol) at 0° C. The mixture was stirred overnight at rt. The mixture was diluted with water (50 mL) and extracted with DCM (3×100 mL). The combined organic layer was washed water, brine, dried over anhydrous Na₂SO₄, filtered and concentrated under vacuum to afford product (1.58 g, 68%). [M+H]⁺=396.2

### Step 4: 3-cyano-N-(2-((1S,4r)-4-(4-(2-(((2S)-1-((2,6-dioxopiperidin-3-yl)amino)-1-oxo-3-phenylpropan-2-yl)amino)-2-oxoethyl)piperazin-1-yl)cyclohexyl)-6-(2-hydroxypropan-2-yl)-2H-indazol-5-yl)pyrrolo[1,2-b]pyridazine-7-carboxamide

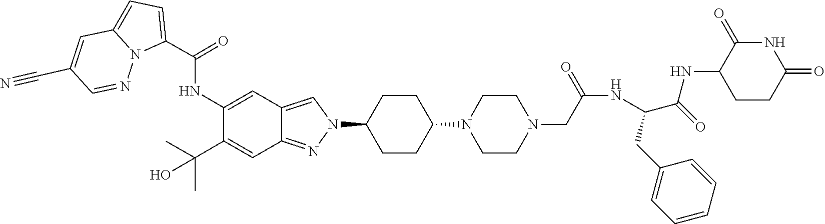 [SMILES: CC(C)(O)c1cc2nn([C@H]3CC[C@H](N4CCN(CC(=O)N[C@@H](Cc5ccccc5)C(=O)NC5CCC(=O)NC5=O)CC4)CC3)cc2cc1NC(=O)c1ccc2cc(C#N)cnn12]

To a solution of 3-cyano-N-(6-(2-hydroxypropan-2-yl)-2-((1r,4r)-4-(piperazin-1-yl)cyclohexyl)-2H-indazol-5-yl)pyrrolo[1,2-b]pyridazine-7-carboxamide (105.2 mg, 0.2 mmol) in DMF (10 mL) was added DIEA (193.5 mg, 0.6 mmol) and (2S)-2-(2-bromoacetamido)-N-(2,6-dioxopiperidin-3-yl)-3-phenylpropanamide (79 mg, 0.2 mmol). The mixture was stirred overnight at rt. The mixture was diluted with water (30 mL) and extracted with DCM (3×50 mL). The combined organic layers were dried over anhydrous Na₂SO₄, filtered and evaporated in vacuum. The residue was purified by Prep-HPLC (C-18 column chromatography (0.1% FA in water:acetonitrile=90:10˜60:40 gradient elution) to give the desired product (48.62 mg, 28.87%).

¹H NMR (500 MHz, DMSO) δ 12.02 (s, 1H), 10.86 (s, 1H), 8.94 (d, J=5.0 Hz, 1H), 8.74 (d, J=5.0 Hz, 1H), 8.55 (s, 1H), 8.52-8.47 (m, 1H), 8.34 (s, 1H), 7.72 (d, J=5 Hz, 1H), 7.70-7.63 (m, 1H), 7.58 (s, 1H), 7.30-719 (m, 5H), 7.09 (d, J=5 Hz, 1H), 5.72 (s, 1H), 4.71-4.53 (m, 2H), 4.46-4.40 (m, 1H), 3.14-3.01 (m, 1H), 2.99-2.88 (m, 2H), 2.80-2.66 (m, 2H), 2.56-2.52 (m, 2H), 2.48-2.15 (m, 10H), 2.02-1.85 (m, 6H), 1.63 (s, 6H), 1.54-1.42 (m, 2H). [M+H]⁺=842.53

### Example 440: 3-cyano-N-(2-((1r,4r)-4-(4-(4-(3-((2,6-dioxopiperidin-3-yl)amino)-3-oxopropyl)phenyl)piperazin-1-yl)cyclohexyl)-6-(2-hydroxypropan-2-yl)-2H-indazol-5-yl)pyrrolo[1,2-b]pyridazine-7-carboxamide

### Step 1: tert-butyl 3-(4-(4-((1r,4r)-4-(5-(3-cyanopyrrolo[1,2-b]pyridazine-7-carboxamido)-6-(2-hydroxypropan-2-yl)-2H-indazol-2-yl)cyclohexyl)piperazin-1-yl)phenyl)propanoate

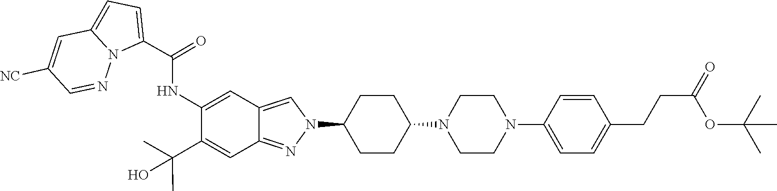 [SMILES: CC(C)(C)OC(=O)CCc1ccc(N2CCN([C@H]3CC[C@H](n4cc5cc(NC(=O)c6ccc7cc(C#N)cnn67)c(C(C)(C)O)cc5n4)CC3)CC2)cc1]

To a solution of 3-cyano-N-(6-(2-hydroxypropan-2-yl)-2-((1r,4r)-4-(piperazin-1-yl)cyclohexyl)-2H-indazol-5-yl)pyrrolo[1,2-b]pyridazine-7-carboxamide (263 mg, 0.5 mmol) and tert-butyl 3-(4-bromophenyl)propanoate (170.4 mg, 0.6 mmol) in 1,4-dioxane (15 mL) was added Cs₂CO₃ (489 mg, 1.5 mmol) and Ruphos Pd G3 (42 mg, 0.05 mmol). The mixture was stirred overnight at 100° C. The mixture concentrated under vacuum. The residue was purified on silica gel column chromatography (DCM:MeOH=15:1) to afford product (204.4 mg, 56%). [M+H]⁺=731.4

### Step 2: 3-(4-(4-((1r,4r)-4-(5-(3-cyanopyrrolo[1,2-b]pyridazine-7-carboxamido)-6-(2-hydroxypropan-2-yl)-2H-indazol-2-yl)cyclohexyl)piperazin-1-yl)phenyl)propanoic acid

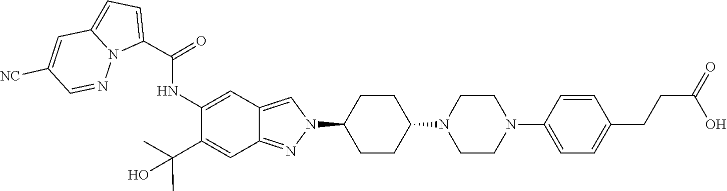 [SMILES: CC(C)(O)c1cc2nn([C@H]3CC[C@H](N4CCN(c5ccc(CCC(=O)O)cc5)CC4)CC3)cc2cc1NC(=O)c1ccc2cc(C#N)cnn12]

To a stirred solution of tert-butyl 3-(4-(4-((1r,4r)-4-(5-(3-cyanopyrrolo[1,2-b]pyridazine-7-carboxamido)-6-(2-hydroxypropan-2-yl)-2H-indazol-2-yl)cyclohexyl)piperazin-1-yl)phenyl)propanoate (204.4 mg, 0.28 mmol) in DCM (10 mL) was added TFA (2 mL). The mixture was stirred for 2 h at rt. The mixture was concentrated under vacuum to afford product (200 mg crude). [M+H]⁺=675.3

### Step 3: 3-cyano-N-(2-((1r,4r)-4-(4-(4-(3-((2,6-dioxopiperidin-3-yl)amino)-3-oxopropyl)phenyl)piperazin-1-yl)cyclohexyl)-6-(2-hydroxypropan-2-yl)-2H-indazol-5-yl)pyrrolo[1,2-b]pyridazine-7-carboxamide

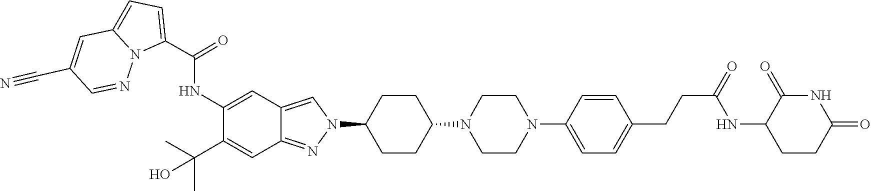 [SMILES: CC(C)(O)c1cc2nn([C@H]3CC[C@H](N4CCN(c5ccc(CCC(=O)NC6CCC(=O)NC6=O)cc5)CC4)CC3)cc2cc1NC(=O)c1ccc2cc(C#N)cnn12]

To a solution of 3-(4-(4-((1r,4r)-4-(5-(3-cyanopyrrolo[1,2-b]pyridazine-7-carboxamido)-6-(2-hydroxypropan-2-yl)-2H-indazol-2-yl)cyclohexyl)piperazin-1-yl)phenyl)propanoic acid (200 mg crude, 0.28 mmol) and 3-aminopiperidine-2,6-dione (53.76 mg, 0.42 mmol) in DCM (10 mL) was added DIEA (108.36 mg, 0.84 mmol) and HATU (106.4 mg, 0.28 mmol). The mixture was stirred for 2 h at rt. The mixture was diluted with water (10 mL) and extracted with DCM (3×20 mL). The combined organic layers were dried over anhydrous Na₂SO₄, filtered and evaporated in vacuum. The residue was purified by Prep-HPLC (C-18 column chromatography (0.1% FA in water:acetonitrile=90:10˜60:40 gradient elution) to give the desired product (29.12 mg, 28.87%).

¹H NMR (500 MHz, DMSO) δ 12.03 (s, 1H), 10.79 (s, 1H), 8.94 (d, J=5.0 Hz, 1H), 8.74 (d, J=5.0 Hz, 1H), 8.56 (s, 1H), 8.35 (s, 1H), 8.19 (d, J=5 Hz, 1H), 7.72 (d, J=5 Hz, 1H), 7.58 (s, 1H), 7.11-7.05 (m, 3H), 6.85 (d, J=5 Hz, 2H), 5.72 (s, 1H), 4.55 (dd, J=5 Hz, 10 Hz, 1H), 4.48-4.41 (m, 1H), 3.12-3.04 (m, 4H), 2.77-2.66 (m, 7H), 2.48-2.44 (m, 2H), 2.41-2.35 (m, 2H), 2.23-2.16 (m, 2H), 2.04-1.84 (m, 6H), 1.63 (s, 6H), 1.57-1.47 (m, 2H). [M+H]⁺=785.59

### Example 422: 3-cyano-N-(2-((1R,4r)-4-((3R)-4-(1-(6-(2,6-dioxopiperidin-3-yl)-2-methylpyridin-3-yl)piperidin-4-yl)-3-(methoxymethyl)piperazin-1-yl)cyclohexyl)-6-(2-hydroxypropan-2-yl)-2H-indazol-5-yl)pyrrolo[1,2-b]pyridazine-7-carboxamide

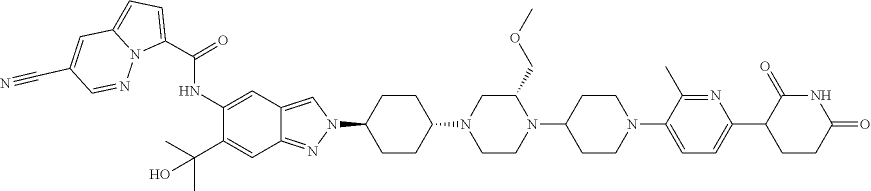 [SMILES: COC[C@H]1CN([C@H]2CC[C@H](n3cc4cc(NC(=O)c5ccc6cc(C#N)cnn56)c(C(C)(C)O)cc4n3)CC2)CCN1C1CCN(c2ccc(C3CCC(=O)NC3=O)nc2C)CC1]

The title compound (45 mg, 15.14%) was prepared in a manner similar to that described in Example 290. ¹H NMR (500 MHz, DMSO) δ 12.02 (s, 1H), 10.78 (s, 1H), 8.94 (d, J=5.0 Hz, 1H), 8.74 (d, J=5.0 Hz, 1H), 8.55 (s, 1H), 8.33 (s, 1H), 7.72 (d, J=5.0 Hz, 1H), 7.58 (s, 1H), 7.36 (d, J=10.0 Hz, 1H), 7.10-7.09 (m, 2H), 5.72 (s, 1H), 4.43-4.40 (m, 1H), 3.90-3.87 (m, 1H), 3.54-3.51 (m, 1H), 3.27 (s, 3H), 3.12-3.10 (m, 2H), 2.89-2.85 (m, 1H), 2.80-2.54 (m, 9H), 2.43-2.34 (m, 6H), 2.24-2.16 (m, 3H), 2.09-2.06 (m, 1H), 1.98-1.86 (m, 5H), 1.75-1.71 (m, 2H), 1.63 (s, 6H), 1.57-1.44 (m, 3H). [M+H]+=856.7.

### Example 423: 3-cyano-N-(2-((1S,4r)-4-((2S)-4-(1-(6-(2,6-dioxopiperidin-3-yl)-2-methylpyridin-3-yl)piperidin-4-yl)-2-(methoxymethyl)piperazin-1-yl)cyclohexyl)-6-(2-hydroxypropan-2-yl)-2H-indazol-5-yl)pyrrolo[1,2-b]pyridazine-7-carboxamide

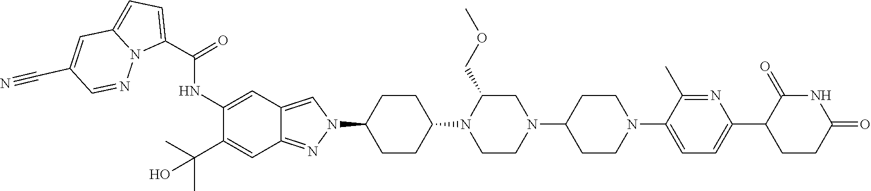 [SMILES: COC[C@@H]1CN(C2CCN(c3ccc(C4CCC(=O)NC4=O)nc3C)CC2)CCN1[C@H]1CC[C@H](n2cc3cc(NC(=O)c4ccc5cc(C#N)cnn45)c(C(C)(C)O)cc3n2)CC1]

The title compound (96 mg, 32.04%) was prepared in a manner similar to that described in Example 290. ¹H NMR (500 MHz, DMSO) δ 12.02 (s, 1H), 10.78 (s, 1H), 8.94 (d, J=5.0 Hz, 1H), 8.74 (d, J=5.0 Hz, 1H), 8.55 (s, 1H), 8.34 (s, 1H), 7.72 (d, J=5.0 Hz, 1H), 7.57 (s, 1H), 7.36 (d, J=10.0 Hz, 1H), 7.11-7.09 (m, 2H), 5.72 (s, 1H), 4.43-4.40 (m, 1H), 3.90-3.87 (m, 1H), 3.54-3.51 (m, 1H), 3.43-3.38 (m, 1H), 3.27 (s, 3H), 3.12-3.10 (m, 2H), 2.89-2.72 (m, 5H), 2.63-2.54 (m, 5H), 2.39-2.17 (m, 9H), 2.09-1.86 (m, 6H), 1.81-1.77 (m, 1H), 1.71-1.55 (m, 9H), 1.46-1.41 (m, 1H). [M+H]⁺=856.7.

### Example 424: 6-cyano-N-(2-((1R,4r)-4-(4-(1-(4-((R)-2,6-dioxopiperidin-3-yl)-3,5-difluorophenyl)azetidin-3-yl)piperazin-1-yl)cyclohexyl)-2H-indazol-5-yl)pyrazolo[1,5-a]pyrimidine-3-carboxamide

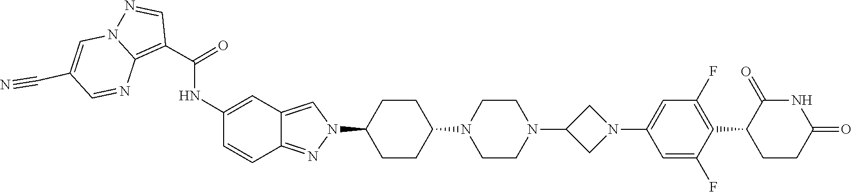 [SMILES: N#Cc1cnc2c(C(=O)Nc3ccc4nn([C@H]5CC[C@H](N6CCN(C7CN(c8cc(F)c([C@H]9CCC(=O)NC9=O)c(F)c8)C7)CC6)CC5)cc4c3)cnn2c1]

The title compound (0.31 mg, 0.41%) was prepared in a manner similar to that described in Example 290. ¹H NMR (500 MHz, DMSO) δ10.86 (s, 1H), 10.22 (s, 1H), 9.82 (s, 1H), 9.16 (s, 1H), 8.93 (s, 1H), 8.37 (s, 1H), 7.57 (s, 1H), 8.30 (s, 1H), 7.62 (d, J=10.0 Hz, 1H), 7.35 (d, J=10.0 Hz, 1H), 6.12 (d, J=10.0 Hz, 2H), 4.43-4.40 (m, 1H), 4.05-4.01 (m, 1H), 3.92-3.90 (m, 2H), 3.65-3.64 (m, 2H), 3.29-3.27 (m, 2H), 2.79-2.75 (m, 1H), 2.57-2.56 (m, 2H), 2.46-2.36 (m, 6H), 2.21-2.17 (m, 2H), 2.09-2.06 (m, 2H), 2.01-1.92 (m, 5H), 1.50-1.46 (m, 2H). [M+H]⁺=748.5.

### Example 441: N-(2-((1r,4r)-4-(4-((E)-3-(adamantan-1-yl)acryloyl)piperazin-1-yl)cyclohexyl)-6-(2-hydroxypropan-2-yl)-2H-indazol-5-yl)-3-cyanopyrrolo[1,2-b]pyridazine-7-carboxamide

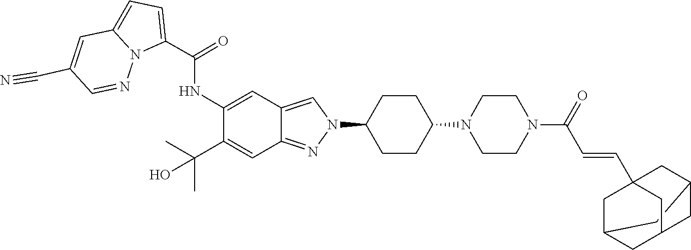 [SMILES: CC(C)(O)c1cc2nn([C@H]3CC[C@H](N4CCN(C(=O)/C=C/C56CC7CC(CC(C7)C5)C6)CC4)CC3)cc2cc1NC(=O)c1ccc2cc(C#N)cnn12]

The title compound (79 mg, 58%) was prepared in a manner similar to that described in Example 178. ¹H NMR (500 MHz, DMSO) δ 12.02 (s, 1H), 8.94 (d, J=5.0 Hz, 1H), 8.74 (d, J=5.0 Hz, 1H), 8.55 (s, 1H), 8.33 (s, 1H), 7.72 (d, J=5.0 Hz, 1H), 7.57 (s, 1H), 7.09 (d, J=5.0 Hz, 1H), 6.54 (d, J=15 Hz, 1H), 6.22 (d, J=15 Hz, 1H), 5.72 (s, 1H), 4.44-4.42 (m, 1H), 3.54-3.50 (m, 4H), 2.54-2.44 (m, 4H), 2.18-2.17 (m, 2H), 1.98-1.91 (m, 7H), 1.72-1.61 (m, 19H), 1.53-1.47 (m, 2H). [M+H]⁺=715.66

### Example 438 3-cyano-N-(2-((1r,4r)-4-((4-(2-((2-(2,6-dioxopiperidin-3-yl)-1,3-dioxoisoindolin-4-yl)amino)ethyl)piperazin-1-yl)methyl)cyclohexyl)-6-(2-hydroxypropan-2-yl)-2H-indazol-5-yl)pyrrolo[1,2-b]pyridazine-7-carboxamide

### Step 1: tert-butyl 4-(((1r,4r)-4-(5-(3-cyanopyrrolo[1,2-b]pyridazine-7-carboxamido)-6-(2-hydroxypropan-2-yl)-2H-indazol-2-yl)cyclohexyl)methyl)piperazine-1-carboxylate

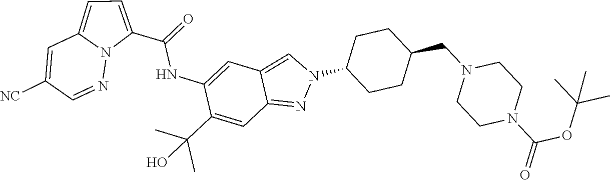 [SMILES: CC(C)(C)OC(=O)N1CCN(C[C@H]2CC[C@H](n3cc4cc(NC(=O)c5ccc6cc(C#N)cnn56)c(C(C)(C)O)cc4n3)CC2)CC1]

To a stirred solution of 3-cyanopyrrolo[1,2-b]pyridazine-7-carboxylic acid (226 mg, 1.21 mmol) and tert-butyl 4-(((1r,4r)-4-(5-amino-6-(2-hydroxypropan-2-yl)-2H-indazol-2-yl)cyclohexyl)methyl)piperazine-1-carboxylate synthesized in a manner similar to that described in Example 157 (570 mg, 1.21 mmol) in DMF (15 mL) was added HATU (552 mg, 1.45 mmol) and DIEA (468 mg, 3.63 mmol). The solution was stirred at rt for 4 h. Then the solution was diluted with EA (30 mL), washed with water and brine. The organic layer was dried over Na₂SO₄ and concentrated under vacuum. The residue was purified with silica gel column, DCM:MeOH=7:1) to yield the product (700 mg, 90.4%). [M+H]⁺=641.4.

### Step 2: 3-cyano-N-(6-(2-hydroxypropan-2-yl)-2-((1r,4r)-4-(piperazin-1-ylmethyl)cyclohexyl)-2H-indazol-5-yl)pyrrolo[1,2-b]pyridazine-7-carboxamide

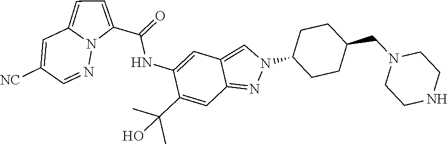 [SMILES: CC(C)(O)c1cc2nn([C@H]3CC[C@H](CN4CCNCC4)CC3)cc2cc1NC(=O)c1ccc2cc(C#N)cnn12]

To a stirring solution of tert-butyl 4-(((1r,4r)-4-(5-(3-cyanopyrrolo[1,2-b]pyridazine-7-carboxamido)-6-(2-hydroxypropan-2-yl)-2H-indazol-2-yl)cyclohexyl)methyl)piperazine-1-carboxylate (700 mg, 1.09 mmol) in MeOH (10 mL) was added 12N HCl (5 mL). The reaction mixture was stirred at rt for 1 hours and to afford the product (390 mg, 66%). [M+H]⁺=541.3.

### Step 3: 3-cyano-N-(2-((1r,4r)-4-((4-(2-((2-(2,6-dioxopiperidin-3-yl)-1,3-dioxoisoindolin-4-yl)amino)ethyl)piperazin-1-yl)methyl)cyclohexyl)-6-(2-hydroxypropan-2-yl)-2H-indazol-5-yl)pyrrolo[1,2-b]pyridazine-7-carboxamide

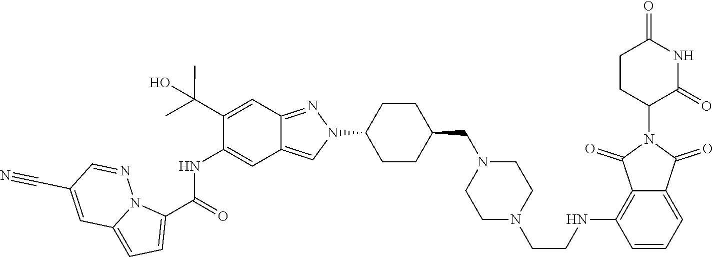 [SMILES: CC(C)(O)c1cc2nn([C@H]3CC[C@H](CN4CCN(CCNc5cccc6c5C(=O)N(C5CCC(=O)NC5=O)C6=O)CC4)CC3)cc2cc1NC(=O)c1ccc2cc(C#N)cnn12]

A solution of 3-cyano-N-(6-(2-hydroxypropan-2-yl)-2-((1r,4r)-4-(piperazin-1-ylmethyl)cyclohexyl)-2H-indazol-5-yl)pyrrolo[1,2-b]pyridazine-7-carboxamide (30 mg, 0.056 mmol), 2-((2-(2,6-dioxopiperidin-3-yl)-1,3-dioxoisoindolin-4-yl)amino)ethyl methanesulfonate (29 mg, 0.072 mmol), KI (18 mg, 0.111 mmol), DIEA (22 mg, 0.167 mmol) and DMSO (0.3 mL) in Acetonitrile (1 mL) was stirred at 80° C. for 16 hours, Then the solution was diluted with EA (30 mL), washed with water and brine. The organic layer was dried over Na₂SO₄ and concentrated under vacuum. The residue was purified by PREP-HPLC (C-18 column chromatography (0.1% FA in water:acetonitrile=85:15˜65:35 gradient elution) to give the desired product (9.99 mg, 22%).

¹H NMR (500 MHz, CD₃OD) δ 12.14 (s, 1H), 8.68 (d, J=5 Hz, 1H), 8.54 (d, J=5 Hz, 1H), 8.42 (s, 1H), 8.25 (s, 1H), 7.82 (d, J=5 Hz, 1H), 7.69 (s, 1H), 7.61-7.58 (m, 1H), 7.11-7.07 (m, 3H), 5.09-5.05 (m, 1H), 4.52-4.48 (m, 1H), 3.58-3.47 (m, 4H), 3.22-2.99 (m, 6H), 2.90-2.83 (m, 3H), 2.78-2.60 (m, 4H), 2.34-2.27 (m, 2H), 2.15-1.97 (m, 6H), 1.71 (s, 6H), 1.42-1.34 (m, 2H). [M+H]⁺=840.4.

### Example 420: 3-cyano-N-(2-((1r,4r)-4-((4-(1-(6-(2,6-dioxopiperidin-3-yl)-2-methylpyridin-3-yl)piperidin-4-yl)piperazin-1-yl)methyl)cyclohexyl)-6-(2-hydroxypropan-2-yl)-2H-indazol-5-yl)pyrrolo[1,2-b]pyridazine-7-carboxamide

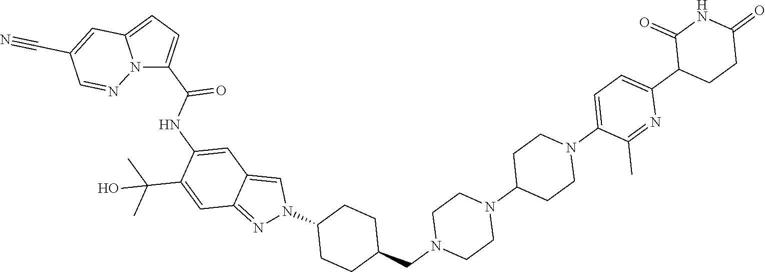 [SMILES: Cc1nc(C2CCC(=O)NC2=O)ccc1N1CCC(N2CCN(C[C@H]3CC[C@H](n4cc5cc(NC(=O)c6ccc7cc(C#N)cnn67)c(C(C)(C)O)cc5n4)CC3)CC2)CC1]

The title compound was prepared in a manner similar to that described in Example 290. ¹H NMR (500 MHz, DMSO) δ 12.02 (s, 1H), 10.78 (s, 1H), 8.94 (d, J=5 Hz, 1H), 8.74 (d, J=5 Hz, 1H), 8.55 (s, 1H), 8.34 (s, 1H), 7.72 (d, J=5 Hz, 1H), 7.57 (s, 1H), 7.36 (d, J=5 Hz, 1H), 7.10-7.09 (m, 2H), 5.72 (s, 1H), 4.44-4.40 (m, 1H), 3.90-3.87 (m, 1H), 3.11 (d, J=10 Hz, 2H), 2.63-2.55 (m, 9H), 2.38-2.30 (m, 7H), 2.25-2.13 (m, 5H), 2.10-2.04 (m, 1H), 1.95-1.86 (m, 6H), 1.63-1.51 (m, 9H), 1.15-1.08 (m, 2H). [M+H]⁺=826.4.

### Example 421: 3-cyano-N-(2-((1r,4r)-4-((4-(1-(4-((R)-2,6-dioxopiperidin-3-yl)-3,5-difluorophenyl)azetidin-3-yl)piperazin-1-yl)methyl)cyclohexyl)-6-(2-hydroxypropan-2-yl)-2H-indazol-5-yl)pyrrolo[1,2-b]pyridazine-7-carboxamide

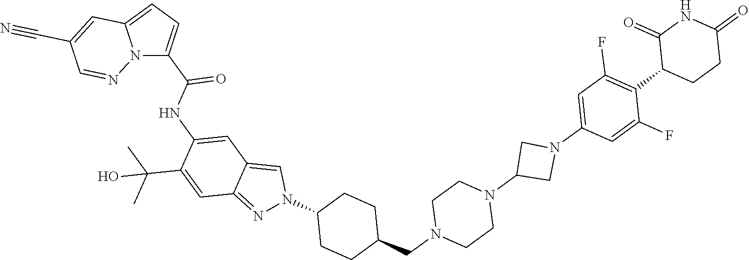 [SMILES: CC(C)(O)c1cc2nn([C@H]3CC[C@H](CN4CCN(C5CN(c6cc(F)c([C@H]7CCC(=O)NC7=O)c(F)c6)C5)CC4)CC3)cc2cc1NC(=O)c1ccc2cc(C#N)cnn12]

The title compound was prepared in a manner similar to that described in Example 290. ¹H NMR (500 MHz, DMSO) δ 12.02 (s, 1H), 10.86 (s, 1H), 8.94 (d, J=5 Hz, 1H), 8.74 (d, J=5 Hz, 1H), 8.55 (s, 1H), 8.34 (s, 1H), 7.72 (d, J=5 Hz, 1H), 7.57 (s, 1H), 7.10 (d, J=5 Hz, 1H), 6.12 (d, J=10 Hz, 2H), 5.72 (s, 1H), 4.45-4.40 (m, 1H), 4.05-4.01 (m, 1H), 3.92-3.89 (m, 2H), 3.65-3.62 (m, 2H), 3.26-3.23 (m, 1H), 2.81-2.74 (m, 1H), 2.49-2.28 (m, 9H), 2.17-2.04 (m, 5H), 1.95-1.88 (m, 5H), 1.63 (s, 7H), 1.15-1.10 (m, 2H). [M+H]⁺=819.4.

### Example 436: 3-cyano-N-(2-((1r,4r)-4-((4-(1-(2-(2,6-dioxopiperidin-3-yl)-1,3-dioxoisoindolin-5-yl)piperidin-4-yl)piperazin-1-yl)methyl)cyclohexyl)-6-(2-hydroxypropan-2-yl)-2H-indazol-5-yl)pyrrolo[1,2-b]pyridazine-7-carboxamide

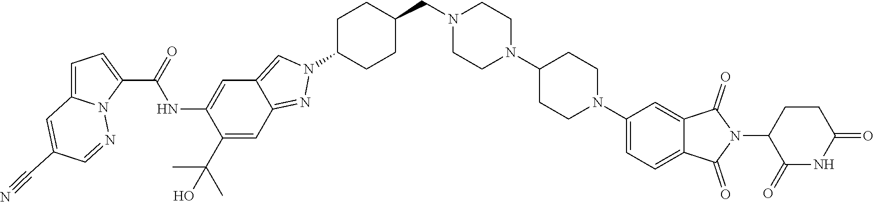 [SMILES: CC(C)(O)c1cc2nn([C@H]3CC[C@H](CN4CCN(C5CCN(c6ccc7c(c6)C(=O)N(C6CCC(=O)NC6=O)C7=O)CC5)CC4)CC3)cc2cc1NC(=O)c1ccc2cc(C#N)cnn12]

The title compound was prepared in a manner similar to that described in Example 290. ¹H NMR (500 MHz, DMSO) δ 12.02 (s, 1H), 11.07 (s, 1H), 8.94 (d, J=5 Hz, 1H), 8.74 (d, J=5 Hz, 1H), 8.55 (s, 1H), 8.33 (s, 1H), 7.72 (d, J=5 Hz, 1H), 7.66 (d, J=5 Hz, 1H), 7.57 (s, 1H), 7.32 (s, 1H), 7.25 (d, J=5 Hz, 1H), 7.09 (d, J=5 Hz, 1H), 5.72 (s, 1H), 5.08-5.05 (m, 1H), 4.44-4.39 (m, 1H), 4.07-4.05 (m, 2H), 2.99-2.86 (m, 3H), 2.64-2.51 (m, 5H), 2.42-2.32 (m, 4H), 2.14-2.10 (m, 4H), 2.03-1.99 (m, 1H), 1.94-1.84 (m, 7H), 1.63 (s, 8H), 1.49-1.42 (m, 2H), 1.14-1.06 (m, 2H). [M+H]⁺=880.4.

### Biological Assays

### IRAK4 Degradation Assay

IRAK4 degradation was measured using a TR-FRET-based method (Degorce, François, et al. Current chemical genomics. 2009, 3: 22). Karpas299 maintained in RPMI 1640 medium (Thermo Scientific) supplemented with 20% fetal bovine serum (FBS, Thermo Scientific), 100 units/mL penicillin and 0.1 mg/mL streptomycin (Thermo Scientific) were seeded into 96-well plates at the density of 0.2 million cells per well and then treated with a series dilution of compounds. After 24 hours or 5 hours treatment, cells were pelleted via centrifugation and 40 μL lysis buffer (Cisbio) was added to each well after removal of the supernatant. Plates were then incubated at room temperature with shaking for 30 minutes. A total of 16 μL of cell lysates from each well of a 96-well plate was transferred to a 384-well white assay plate. IRAK4 level was quantitated using the HTRF kit as described by the manufacturer manual (Cisbio). FRET signals were measured using a PHERAstar FSX reader (BMG Labtech).

### TNF-α Production Assay in THP-1 Cells

THP-1 cells maintained in RPMI 1640 medium (Thermo Scientific) supplemented with 10% FBS (Thermo Scientific), 100 units/mL penicillin and 0.1 mg/mL streptomycin (Thermo Scientific) were seeded into 96-well plates at the density of 0.2 million cells per well and then treated with compounds for 24 h. Lipopolysaccharide was subsequently added at the final concentration of 1 μg/mL for 24 h. Culture supernatant was collected for measurement of TNF-α concentration by a TR-FRET-based method (Degorce, François, et al. Current chemical genomics. 2009, 3: 22) as described by the manufacturer manual (Cisbio). FRET signals were measured using a PHERAstar FSX reader (BMG Labtech).

### IL-6 Production Assay in Human PBMC

Frozen human PBMC (AllCells) were thawed in RPMI 1640 medium (Thermo Scientific) supplemented with 10% heat-inactivated FBS (Thermo Scientific) and recovered at 37° C. overnight. The next day, PBMC were seeded into 96-well plates at the density of 0.2 million cells per well and treated with compounds for 24 h. Then lipopolysaccharide was added at the final concentration of 10 ng/mL for 5 h. Culture supernatant was collected for subsequent measurement of IL-6 concentration by a TR-FRET-based method (Degorce, François, et al. Current chemical genomics. 2009, 3: 22) as described by the manufacturer manual (Cisbio). FRET signals were measured with PHERAstar FSX reader (BMG Labtech).

IRAK4 Degradation Assay with Human Serum

IRAK4 degradation was measured using a TR-FRET-based method (Degorce, François, et al. Current chemical genomics. 2009, 3: 22). Karpas299 resuspended in Human serum AB (GEMINI, 100-512) were seeded into 96-well plates at the density of 0.2 million cells per well and then treated with a series dilution of compounds. After 24 hrs treatment, cells were pelleted via centrifugation and washed with PBS, add 40 μL lysis buffer (Cisbio) to each well after removal of the supernatant. Plates were then incubated at room temperature with shaking for 30 minutes. A total of 16 μL of cell lysates from each well of a 96-well plate was transferred to a 384-well white assay plate. IRAK4 level was quantitated using the HTRF kit as described by the manufacturer manual (Cisbio). FRET signals were measured using a PHERAstar FSX reader (BMG Labtech).

### HTRF Assay

After treatment, add HTRF lysis buffer to each well; seal the plate and incubate 1 hour at room temperature on a plate shaker; Once the cells are lysed, 16 μL of cell lysate are transferred to a PE 384-well HTRF detection plate; 4 μL of pre-mixed HTRF antibodies are added to each well; Cover the plate with a plate sealer, spin 1000 rpm for 1 min, Incubate overnight at room temperature; Read on BMG PheraStar with HTRF protocol (337 nm-665 nm-620 nm).

The inhibition (degradation) percentage of the compound was calculated by the following equation:

$$Inhibition percentage of Compound = 100 - 100 \times ( Signal - low control ) / ( High control - low control ) ,$$

wherein signal=each test compound group
Low control=only lysis buffer without cells, indicating that IRAK4 is completely degraded;
High control=Cell group with added DMSO and without compound, indicating microplate readings without IRAK4 degradation;
Dmax is the maximum percentage of inhibition (degradation).

The IC₅₀ (DC₅₀) value of a compound can be obtained by fitting the following equation

$$Y = Bottom + ( TOP - Bottom ) / ( 1 + ( ( {IC}_{50} / X ) \hat{} hillslope ) )$$

Wherein, X and Y are known values, and IC₅₀, Hillslope, Top and Bottom are the parameters obtained by fitting with software. Y is the inhibition percentage (calculated from the equation), X is the concentration of the compound; IC₅₀ is the concentration of the compound when the 5000 inhibition is reached. The smaller the IC₅₀ value is, the stronger the inhibitory ability of the compound is. Vice versa, the higher the IC₅₀ value is, the weaker the ability the inhibitory ability of the compound is; Hillslope represents the slope of the fitted curve, generally around 1 *; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software.

TABLE 1
IRAK4 Degradation (Karpas-299) result
for Example 1 to Example 441

| | IRAK4 in Karpas-299 (24 h) | | IRAK4 in Karpas-299 (5 h) | |
|---|---|---|---|---|
| Example | DC₅₀ (nM) | Dmax (%) | DC₅₀ (nM) | Dmax (%) |
| 1 | >10000 | 46 | NA | NA |
| 2 | >10000 | 6.5 | NA | NA |
| 3 | >10000 | 9.0 | NA | NA |
| 4 | 6.2 | 95 | NA | NA |
| 5 | 14 | 92 | NA | NA |
| 6 | >10000 | 11 | NA | NA |
| 7 | 137 | 66 | NA | NA |
| 8 | 2.1 | 97 | NA | NA |
| 9 | 2.5 | 95 | NA | NA |
| 10 | 5.4 | 92 | NA | NA |
| 11 | 1.6 | 97 | 2.4 | 94 |
| 12 | 1.8 | 98 | 5.4 | 97 |
| 13 | 2.7 | 94 | 7.5 | 84 |
| 14 | 3.4 | 70 | NA | NA |
| 15 | >10000 | 30 | NA | NA |
| 16 | 0.67 | 98 | 5.7 | 96 |
| 17 | 2.2 | 97 | 8.8 | 96 |
| 18 | 9.7 | 97 | 100 | 95 |
| 19 | 1.0 | 95 | NA | NA |
| 20 | 4.8 | 97 | NA | NA |
| 21 | 4.4 | 98 | NA | NA |
| 22 | 6.8 | 90 | NA | NA |
| 23 | 6.0 | 95 | NA | NA |
| 24 | 2.8 | 98 | NA | NA |
| 25 | 2.5 | 99 | NA | NA |
| 26 | 2.2 | 99 | NA | NA |
| 27 | 2.8 | 99 | NA | NA |
| 28 | 2.5 | 96 | NA | NA |
| 29 | 6.9 | 96 | 162 | 76 |
| 30 | 3.2 | 95 | 5.3 | 76 |
| 31 | 5.8 | 96 | 8.2 | 89 |
| 32 | 2.7 | 95 | 15 | 91 |
| 33 | 3.1 | 99 | 33 | 81 |
| 34 | 3.6 | 95 | 18 | 81 |
| 35 | >10000 | 10 | NA | NA |
| 36 | 15 | 89 | NA | NA |
| 37 | 0.7 | 96 | 9.1 | 93 |
| 38 | 0.4 | 99 | 2.3 | 95 |
| 39 | 0.8 | 99 | 4.6 | 96 |
| 40 | 0.9 | 99 | 23 | 89 |
| 41 | 1.1 | 95 | NA | NA |
| 42 | 2.2 | 95 | 4.9 | 83 |
| 43 | 7.7 | 95 | NA | NA |
| 44 | 9.3 | 99 | NA | NA |
| 45 | 6.3 | 98 | 16 | 88 |
| 46 | 17 | 97 | NA | NA |
| 47 | 0.6 | 98 | NA | NA |
| 50 | 1.5 | 94 | 2.2 | 78 |
| 51 | 1.2 | 96 | 4.8 | 93 |
| 52 | 2.0 | 96 | NA | NA |
| 61 | 5.0 | 96 | 24 | 86 |
| 63 | 15 | 95 | NA | NA |
| 76 | 3.2 | 97 | NA | NA |
| 85 | 5.2 | 95 | NA | NA |
| 86 | 8.9 | 94 | NA | NA |
| 90 | 3.1 | 96 | NA | NA |
| 98 | 0.6 | 99 | NA | NA |
| 99 | 0.4 | 99 | NA | NA |
| 100 | 2.2 | 97 | NA | NA |
| 101 | 1.2 | 97 | NA | NA |
| 102 | 2.0 | 94 | 12 | 61 |
| 103 | 4.4 | 90 | NA | NA |
| 105 | 0.7 | 98 | 3.1 | 97 |
| 106 | 2.4 | 97 | NA | NA |
| 108 | 3.9 | 97 | NA | NA |
| 110 | 0.8 | 98 | 1.1 | 94 |
| 112 | 8.6 | 96 | NA | NA |
| 114 | 1.3 | 96 | NA | NA |
| 115 | 3.8 | 96 | NA | NA |
| 117 | 4.2 | 78 | NA | NA |
| 118 | 2.8 | 85 | NA | NA |
| 120 | 4.2 | 99 | NA | NA |
| 126 | 1.1 | 99 | 8.4 | 92 |
| 127 | 1.3 | 102 | 12 | 94 |
| 128 | 7.2 | 100 | 41 | 90 |
| 129 | 1.9 | 99 | NA | NA |
| 130 | 0.5 | 98 | NA | NA |
| 135 | 1.1 | 98 | 1.6 | 93 |
| 149 | 0.3 | 97 | NA | NA |
| 151 | 0.9 | 99 | NA | NA |
| 152 | 2.1 | 96 | 3.8 | 96 |
| 153 | 1.6 | 97 | NA | NA |
| 157 | 0.4 | 98 | NA | NA |
| 159 | 1.5 | 95 | NA | NA |
| 160 | 0.4 | 99 | 4.0 | 90 |
| 161 | 0.3 | 97 | 5.0 | 91 |
| 162 | 5.7 | 96 | NA | NA |
| 163 | 3.8 | 95 | NA | NA |
| 164 | 2.0 | 97 | NA | NA |
| 165 | 0.7 | 98 | 13 | 97 |
| 166 | 2.6 | 96 | NA | NA |
| 167 | 0.8 | 99 | 5.3 | 94 |
| 168 | 1.3 | 100 | 5.2 | 87 |
| 169 | 4.3 | 99 | 17 | 95 |
| 170 | 6.8 | 96 | NA | NA |
| 171 | 10 | 95 | NA | NA |
| 172 | 1.6 | 94 | NA | NA |
| 173 | 1.8 | 98 | 17 | 99 |
| 174 | 7.7 | 97 | NA | NA |
| 175 | 1.7 | 100 | 12 | 88 |
| 176 | 1.6 | 97 | 12 | 96 |
| 177 | 7.8 | 97 | 44 | 94 |
| 178 | 0.4 | 98 | 0.6 | 97 |
| 181 | 0.4 | 97 | 2.0 | 97 |
| 182 | 0.3 | 98 | 2.3 | 96 |
| 183 | 0.3 | 98 | NA | NA |
| 184 | 0.7 | 96 | NA | NA |
| 185 | 0.7 | 96 | NA | NA |
| 186 | 0.9 | 99 | NA | NA |
| 187 | 0.6 | 96 | NA | NA |
| 193 | 0.4 | 97 | NA | NA |
| 194 | 1.1 | 98 | 1.6 | 93 |
| 195 | 2.8 | 98 | 13 | 97 |
| 196 | 0.6 | 99 | 5.3 | 99 |
| 197 | 4.8 | 96 | NA | NA |
| 198 | 9.0 | 93 | NA | NA |
| 199 | 0.4 | 99 | NA | NA |
| 201 | 0.8 | 96 | NA | NA |
| 207 | 1.9 | 97 | NA | NA |
| 210 | 3.3 | 97 | 6.8 | 97 |
| 212 | 3.6 | 94 | NA | NA |
| 225 | 1.0 | 97 | NA | NA |
| 254 | 4.4 | 96 | 6.7 | 97 |
| 255 | 1.2 | 97 | 2.7 | 96 |
| 257 | 1.7 | 97 | NA | NA |
| 258 | 2.6 | 96 | NA | NA |
| 259 | 0.8 | 98 | NA | NA |
| 263 | 2.6 | 96 | NA | NA |
| 265 | 2.6 | 97 | NA | NA |
| 269 | 1.8 | 97 | NA | NA |
| 271 | 4.0 | 94 | NA | NA |
| 272 | 6.7 | 96 | NA | NA |
| 273 | 5.0 | 95 | NA | NA |
| 274 | 1.6 | 97 | 9.8 | 97 |
| 287 | 1.9 | 94 | NA | NA |
| 288 | 2.3 | 96 | NA | NA |
| 289 | 1.3 | 98 | NA | NA |
| 290 | 0.6 | 98 | NA | NA |
| 291 | 0.6 | 97 | NA | NA |
| 292 | 1.8 | 94 | NA | NA |
| 293 | 9.4 | 96 | NA | NA |
| 294 | 3.4 | 97 | NA | NA |
| 295 | 16 | 87 | NA | NA |
| 296 | 1.8 | 97 | 5.4 | 91 |
| 297 | 1.4 | 97 | 3.4 | 96 |
| 298 | 0.3 | 99 | NA | NA |
| 299 | 0.8 | 98 | NA | NA |
| 300 | 0.9 | 96 | NA | NA |
| 301 | 0.7 | 100 | NA | NA |
| 302 | 0.8 | 98 | NA | NA |
| 303 | 0.4 | 101 | NA | NA |
| 304 | 0.5 | 96 | NA | NA |
| 305 | 1.7 | 96 | NA | NA |
| 306 | 1.3 | 98 | NA | NA |
| 307 | 0.5 | 100 | NA | NA |
| 308 | 1.5 | 97 | NA | NA |
| 309 | 0.4 | 97 | NA | NA |
| 310 | 0.6 | 98 | NA | NA |
| 311 | 0.8 | 98 | NA | NA |
| 312 | 2.4 | 97 | NA | NA |
| 313 | 0.3 | 98 | NA | NA |
| 314 | 0.6 | 98 | NA | NA |
| 315 | 0.6 | 95 | 0.9 | 97 |
| 316 | 1.6 | 97 | NA | NA |
| 317 | 0.8 | 96 | 1.9 | 95 |
| 318 | 1.1 | 91 | NA | NA |
| 319 | 2.0 | 98 | 1.2 | 95 |
| 320 | 1.0 | 99 | NA | NA |
| 321 | 1.4 | 96 | NA | NA |
| 322 | 0.7 | 97 | 12 | 98 |
| 323 | 2.1 | 94 | NA | NA |
| 324 | 1.0 | 95 | NA | NA |
| 325 | 0.9 | 96 | NA | NA |
| 326 | 0.7 | 100 | 1.9 | 97 |
| 327 | 0.9 | 90 | NA | NA |
| 328 | 0.8 | 98 | NA | NA |
| 329 | 0.8 | 96 | NA | NA |
| 330 | 0.6 | 96 | NA | NA |
| 331 | 3.7 | 92 | NA | NA |
| 332 | 0.6 | 97 | 1.5 | 97 |
| 333 | 2.4 | 97 | 3.9 | 98 |
| 334 | 0.6 | 95 | NA | NA |
| 335 | 0.6 | 93 | NA | NA |
| 336 | 0.2 | 100 | NA | NA |
| 337 | 2.1 | 96 | NA | NA |
| 338 | 1.1 | 95 | NA | NA |
| 339 | 2.5 | 95 | NA | NA |
| 340 | 3.4 | 98 | NA | NA |
| 341 | 1.5 | 97 | NA | NA |
| 342 | 1.5 | 102 | NA | NA |
| 343 | 0.1 | 97 | NA | NA |
| 344 | 0.24 | 98 | NA | NA |
| 345 | 0.2 | 98 | NA | NA |
| 346 | 1.4 | 97 | 1.6 | 93 |
| 347 | 1.3 | 96 | 2.2 | 93 |
| 348 | 0.4 | 94 | NA | NA |
| 349 | 2.4 | 97 | 3.9 | 94 |
| 350 | 2.1 | 89 | NA | NA |
| 353 | 0.8 | 95 | NA | NA |
| 354 | 2.4 | 98 | NA | NA |
| 355 | 0.4 | 95 | NA | NA |
| 356 | 1.3 | 98 | NA | NA |
| 357 | 1.1 | 97 | NA | NA |
| 358 | 0.5 | 99 | 1.9 | 95 |
| 359 | 2.4 | 97 | NA | NA |
| 360 | 0.6 | 97 | NA | NA |
| 361 | 4.3 | 97 | NA | NA |
| 362 | 2.5 | 98 | NA | NA |
| 363 | 0.8 | 96 | 1.2 | 95 |
| 364 | 1.3 | 96 | 0.8 | 96 |
| 365 | 1.1 | 93 | NA | NA |
| 366 | 1.4 | 98 | NA | NA |
| 367 | 0.7 | 99 | 3.1 | 97 |
| 369 | 0.6 | 96 | NA | NA |
| 382 | 0.4 | 98 | NA | NA |
| 383 | 1.6 | 94 | NA | NA |
| 384 | 2.2 | 97 | NA | NA |
| 385 | 4.8 | 91 | NA | NA |
| 386 | 3.2 | 94 | NA | NA |
| 387 | 0.8 | 98 | 1.3 | 90 |
| 388 | 1.1 | 97 | NA | NA |
| 389 | 1.0 | 94 | NA | NA |
| 390 | 2.0 | 99 | NA | NA |
| 392 | 0.9 | 97 | NA | NA |
| 395 | 0.8 | 97 | NA | NA |
| 396 | 0.8 | 98 | NA | NA |
| 397 | 1.7 | 95 | NA | NA |
| 398 | 2.3 | 97 | 1.9 | 97 |
| 399 | 0.5 | 96 | NA | NA |
| 400 | 0.5 | 97 | NA | NA |
| 401 | 1.2 | 94 | NA | NA |
| 402 | 0.9 | 95 | NA | NA |
| 403 | 0.7 | 96 | NA | NA |
| 404 | 0.8 | 93 | NA | NA |
| 405 | 0.9 | 96 | NA | NA |
| 409 | 3.5 | 97 | NA | NA |
| 410 | 1.8 | 98 | NA | NA |
| 411 | >10000 | 9 | NA | NA |
| 412 | >10000 | 5 | NA | NA |
| 413 | >1000 | 25 | NA | NA |
| 414 | 1.5 | 96 | NA | NA |
| 415 | 1.4 | 97 | NA | NA |
| 416 | 1.9 | 98 | NA | NA |
| 417 | 0.9 | 95 | NA | NA |
| 418 | 1.8 | 94 | NA | NA |
| 419 | 1.6 | 97 | NA | NA |
| 420 | 2.8 | 97 | NA | NA |
| 421 | 0.8 | 95 | NA | NA |
| 422 | 2.1 | 97 | NA | NA |
| 423 | 1.1 | 98 | NA | NA |
| 424 | >1000 | 42 | NA | NA |
| 425 | 0.6 | 98 | NA | NA |
| 426 | 5.5 | 95 | NA | NA |
| 427 | 2.9 | 96 | NA | NA |
| 428 | 2.9 | 93 | NA | NA |
| 429 | 1.0 | 97 | NA | NA |
| 430 | 1.4 | 97 | NA | NA |
| 431 | 1.6 | 94 | NA | NA |
| 432 | 1.9 | 95 | NA | NA |
| 433 | 0.2 | 99 | NA | NA |
| 434 | 0.2 | 97 | NA | NA |
| 435 | 0.4 | 100 | NA | NA |
| 436 | 10.8 | 96 | NA | NA |
| 437 | 4.7 | 99 | NA | NA |
| 438 | 2.5 | 96 | NA | NA |
| 439 | 251.7 | 58 | NA | NA |
| 440 | 183.1 | 80 | NA | NA |
| 441 | >1000 | 4 | NA | NA |

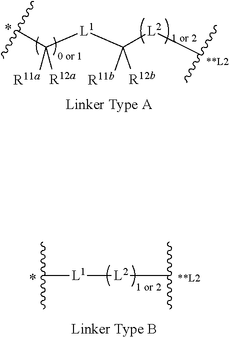

TABLE 2
Degradation (Karpas-299) comparison of selected
compounds with Linker Type A and Linker Type B.

| | Compound (Linker Type A) | | Compound (Linker Type B) | |
|---|---|---|---|---|
| Example | DC₅₀ (nM) | Dmax (%) | DC₅₀ (nM) | Dmax (%) |
| 1 | >10000 | 46 | | |
| 2 | >10000 | 6.5 | | |
| 3 | >10000 | 9.0 | | |
| 6 | >10000 | 11 | | |
| 7 | 137 | 66 | | |
| 35 | >10000 | 10 | | |
| 4 | 6.2 | 95 | | |
| 16 | | | 0.67 | 98 |
| 5 | 14 | 92 | | |
| 37 | | | 0.7 | 96 |
| 36 | 15 | 89 | | |
| 164 | | | 2.0 | 97 |

TABLE 3
IRAK4 Degradation (Karpas-299) with human
serum assay result for selected Examples

IRAK4 degradtion with human serum assay

| Example | DC₅₀ (nM) | Dmax (%) |
|---|---|---|
| 105 | 21 | 98 |
| 106 | 57 | 97 |
| 108 | 28 | 94 |
| 110 | 8.3 | 100 |
| 135 | 7.1 | 100 |
| 151 | 3.9 | 101 |
| 152 | 55 | 98 |
| 153 | 50 | 94 |
| 157 | 21 | 99 |
| 159 | 5.2 | 94 |
| 178 | 17 | 100 |
| 181 | 11 | 101 |
| 182 | 3.7 | 99 |
| 183 | 35 | 95 |
| 184 | 126 | 97 |
| 185 | 14 | 100 |
| 186 | 9.2 | 101 |
| 187 | 11 | 98 |
| 193 | 5.8 | 102 |
| 194 | 6.2 | 96 |
| 195 | 10 | 98 |
| 196 | 8.8 | 93 |
| 199 | 22 | 95 |
| 201 | 11 | 99 |
| 207 | 191 | 97 |
| 210 | 7.0 | 98 |
| 212 | 40 | 95 |
| 254 | 25 | 98 |
| 255 | 7.8 | 96 |
| 257 | 28 | 97 |
| 258 | 21 | 97 |
| 259 | 6.3 | 101 |
| 263 | 15 | 99 |
| 265 | 21 | 99 |
| 269 | 11 | 94 |
| 271 | 11 | 101 |
| 272 | 19 | 94 |
| 273 | 9.3 | 98 |
| 274 | 3.7 | 99 |
| 289 | 19 | 98 |
| 290 | 4.4 | 99 |
| 291 | 48 | 97 |
| 292 | 37 | 95 |
| 293 | 9.4 | 96 |
| 294 | 14 | 98 |
| 296 | 18 | 100 |
| 297 | 6.0 | 99 |
| 298 | 9.8 | 103 |
| 299 | 4.8 | 99 |
| 300 | 2.4 | 102 |
| 301 | 3.2 | 102 |
| 302 | 8.8 | 96 |
| 303 | 16 | 101 |
| 304 | 65 | 97 |
| 305 | 5.1 | 99 |
| 306 | 8.3 | 100 |
| 307 | 3.6 | 99 |
| 308 | 8.9 | 99 |
| 309 | 14 | 100 |
| 310 | 3.2 | 102 |
| 311 | 15 | 99 |
| 312 | 34 | 99 |
| 313 | 24 | 96 |
| 314 | 3.4 | 98 |
| 315 | 18 | 99 |
| 316 | 35 | 95 |
| 317 | 25 | 100 |
| 318 | 39 | 95 |
| 319 | 6.7 | 98 |
| 320 | 4.1 | 98 |
| 321 | 5.5 | 98 |
| 322 | 19 | 101 |
| 323 | 14 | 98 |
| 324 | 23 | 99 |
| 325 | 15 | 91 |
| 326 | 2.9 | 95 |
| 327 | 4.2 | 95 |
| 328 | 19 | 101 |
| 329 | 19 | 98 |
| 330 | 10 | 98 |
| 332 | 10 | 101 |
| 333 | 23 | 93 |
| 334 | 6.3 | 94 |
| 335 | 64 | 99 |
| 336 | 13 | 92 |
| 337 | 10 | 89 |
| 338 | 13 | 92 |
| 339 | 20 | 99 |
| 340 | 16 | 97 |
| 341 | 15 | 96 |
| 342 | 14 | 98 |
| 343 | 2.3 | 102 |
| 344 | 2.7 | 100 |
| 345 | 0.7 | 96 |
| 346 | 5.5 | 99 |
| 347 | 25 | 103 |
| 348 | 6.6 | 95 |
| 349 | 7.7 | 98 |
| 350 | 16 | 97 |
| 353 | 3.5 | 88 |
| 354 | 10 | 93 |
| 355 | 75 | 73 |
| 356 | 14 | 102 |
| 357 | 12 | 97 |
| 358 | 5.4 | 96 |
| 359 | 4.2 | 103 |
| 360 | 1.8 | 98 |
| 361 | 5.0 | 93 |
| 362 | 5.1 | 97 |
| 363 | 5.9 | 97 |
| 364 | 4.6 | 99 |
| 365 | 7.7 | 93 |
| 366 | 14 | 97 |
| 367 | 12 | 96 |
| 369 | 3.0 | 97 |
| 382 | 11 | 96 |
| 383 | 5.8 | 93 |
| 387 | 6.9 | 95 |
| 388 | 12 | 98 |
| 389 | 13 | 100 |
| 390 | 24 | 99 |
| 392 | 11 | 98 |
| 395 | 21 | 99 |
| 396 | 34 | 95 |
| 397 | 12 | 96 |
| 398 | 2.6 | 98 |
| 399 | 11 | 99 |
| 400 | 3.8 | 97 |
| 401 | 7.4 | 94 |
| 402 | 5.5 | 87 |
| 403 | 3.5 | 99 |
| 404 | 5.4 | 94 |
| 409 | 10 | 96 |
| 410 | 18 | 91 |
| 414 | 439 | 97 |
| 415 | 8.6 | 98 |
| 416 | 9.2 | 99 |
| 417 | 4.5 | 95 |
| 418 | 6.1 | 100 |
| 419 | 4.0 | 97 |
| 420 | 17 | 96 |
| 421 | 15 | 97 |
| 422 | 5.2 | 97 |
| 423 | 3.0 | 96 |
| 425 | 19 | 94 |
| 426 | 12 | 95 |
| 427 | 8.4 | 94 |
| 428 | 33 | 94 |
| 429 | 32 | 94 |
| 430 | 15 | 98 |
| 431 | 8.7 | 95 |
| 432 | 14 | 92 |
| 436 | 14 | 85 |
| 437 | 9.4 | 101 |
| 438 | 17 | 96 |

TABLE 4
Cytokine (IL-6/TNF-α) production stimulated
with LPS for selected Examples.

| | IL-6 (PBMC) 5 h | | TNF-α (THP-1) 24 h | |
|---|---|---|---|---|
| Example | IC₅₀ (nM) | IMax (%) | IC₅₀ (nM) | IMax (%) |
| 12 | 4.6 | 91 | 25 | 90 |
| 17 | 6.7 | 93 | 34 | 87 |
| 33 | | | 101 | 53 |
| 34 | 18 | 85 | 77 | 72 |
| 37 | 11 | 90 | 34 | 82 |
| 38 | 2.1 | 91 | 19 | 87 |
| 105 | | | 36 | 78 |
| 110 | 1.5 | 63 | 27 | 83 |
| 114 | | | 53 | 78 |
| 126 | 23 | 90 | 43 | 88 |
| 151 | 7.4 | 53 | 27 | 84 |
| 161 | 4.9 | 90 | 38 | 85 |
| 165 | 7.9 | 91 | 73 | 82 |
| 173 | | | 69 | 79 |
| 176 | | | 120 | 78 |
| 178 | 1.2 | 61 | 6.4 | 92 |
| 181 | | | 30 | 69 |
| 182 | 2.5 | 64 | 33 | 71 |
| 185 | | | 12 | 91 |
| 186 | | | 29 | 89 |
| 193 | | | 20 | 58 |
| 194 | | | 30 | 53 |
| 195 | | | 118 | 72 |
| 196 | 11 | 72 | 20 | 86 |
| 201 | | | 6.7 | 80 |
| 259 | | | 19 | 90 |
| 269 | | | 24 | 89 |
| 274 | 2.0 | 64 | | |
| 290 | 0.8 | 71 | 9.0 | 96 |
| 298 | | | 13 | 93 |
| 299 | 4.0 | 74 | 43 | 94 |
| 301 | | | 42 | 96 |
| 302 | | | 38 | 84 |
| 304 | | | 31 | 88 |
| 309 | | | 31 | 90 |
| 311 | | | 29 | 91 |
| 314 | 2.3 | 51 | 26 | 88 |
| 315 | | | 22 | 93 |
| 317 | 1.0 | 71 | 41 | 87 |
| 319 | | | 39 | 90 |
| 320 | 9.2 | 65 | 43 | 85 |
| 321 | 0.4 | 58 | 55 | 77 |
| 322 | 9.7 | 81 | 42 | 94 |
| 325 | 4.2 | 72 | | |
| 326 | 1.9 | 65 | 13 | 99 |
| 330 | | | 19 | 89 |
| 332 | 1.4 | 70 | 7.6 | 93 |
| 333 | 4.7 | 62 | 10 | 96 |
| 339 | | | 122 | 67 |
| 345 | 3.5 | 60 | | |
| 354 | | | 44 | 96 |
| 356 | | | 12 | 90 |
| 358 | | | 17 | 96 |
| 360 | 0.6 | 67 | | |
| 362 | 6.0 | 71 | | |
| 363 | 2.2 | 67 | | |
| 364 | 0.6 | 74 | 14 | 98 |
| 367 | | | 11 | 96 |
| 387 | 1.7 | 53 | | |
| 398 | 2.4 | 70 | 21 | 97 |
| PF-06650833 | | <50 | 19 | 67 |
| I-417(WO2020113233) | | <50 | | 33 |

It is to be understood that, if any prior art publication is referred to herein; such reference does not constitute an admission that the publication forms a part of the common general knowledge in the art in any country.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1-53. (canceled)

54. A compound selected from the group consisting of: 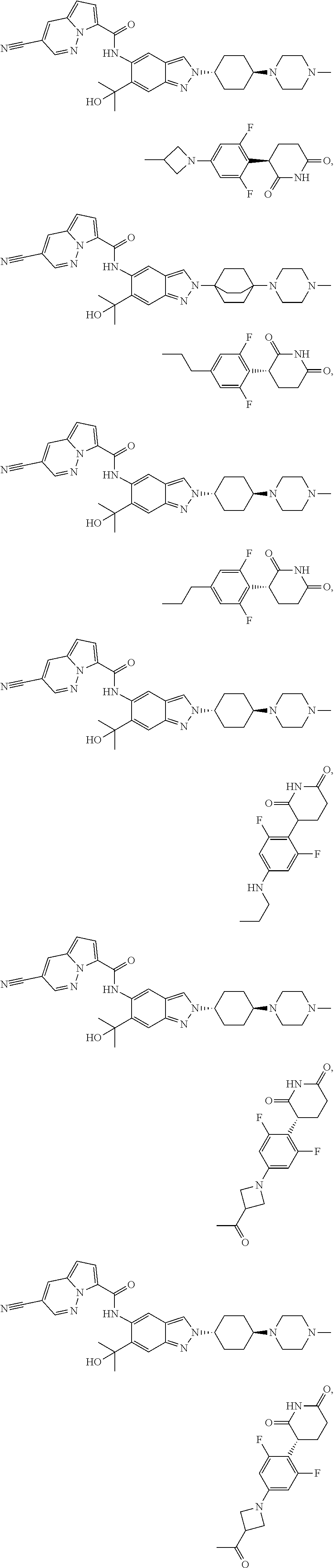 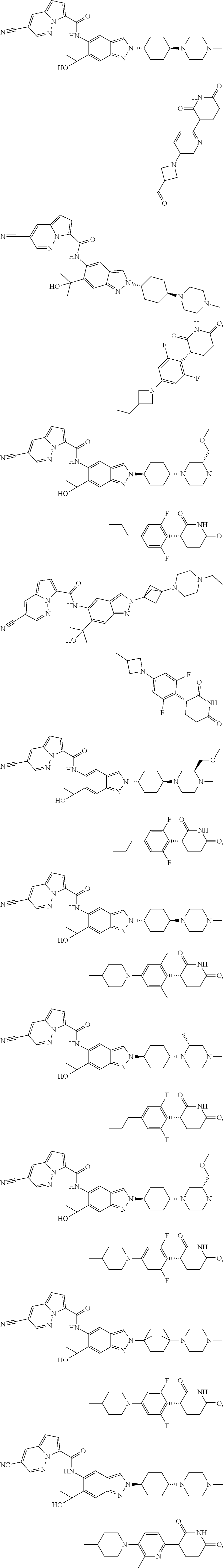 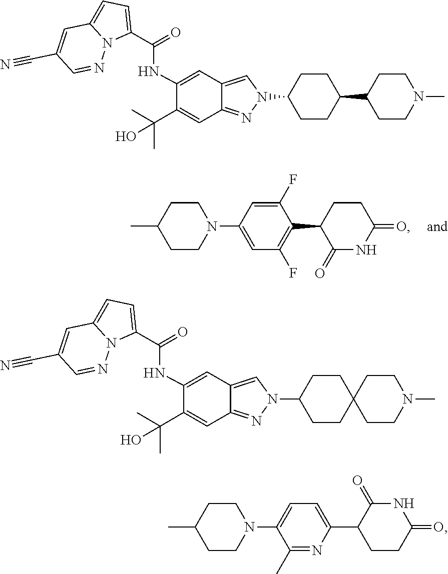
or a pharmaceutically acceptable salt thereof.

55. The compound of claim 54, wherein the compound is 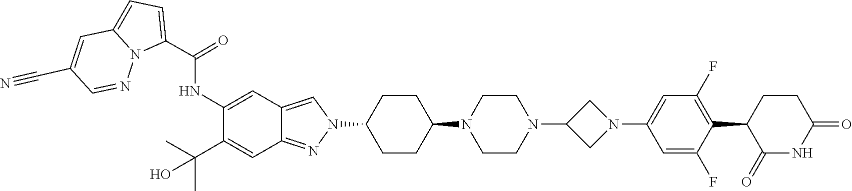 [SMILES: CC(C)(O)c1cc2nn([C@H]3CC[C@H](N4CCN(C5CN(c6cc(F)c([C@H]7CCC(=O)NC7=O)c(F)c6)C5)CC4)CC3)cc2cc1NC(=O)c1ccc2cc(C#N)cnn12]
or a pharmaceutically acceptable salt thereof.

56. The compound of claim 54, wherein the compound is 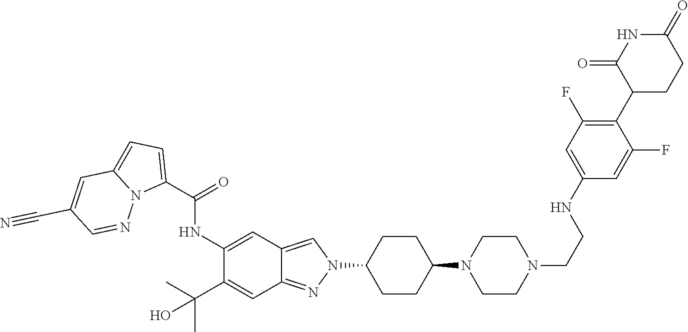 [SMILES: CC(C)(O)c1cc2nn([C@H]3CC[C@H](N4CCN(CCNc5cc(F)c(C6CCC(=O)NC6=O)c(F)c5)CC4)CC3)cc2cc1NC(=O)c1ccc2cc(C#N)cnn12]
or a pharmaceutically acceptable salt thereof.

57. The compound of claim 54, wherein the compound is 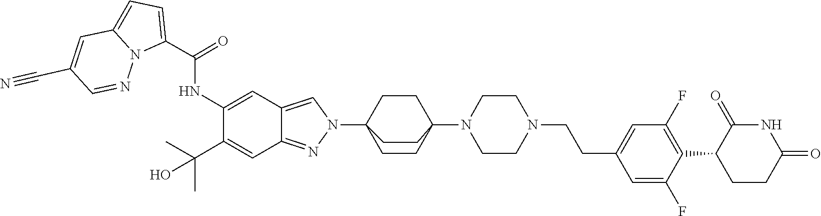 [SMILES: CC(C)(O)c1cc2nn(C34CCC(N5CCN(CCc6cc(F)c([C@H]7CCC(=O)NC7=O)c(F)c6)CC5)(CC3)CC4)cc2cc1NC(=O)c1ccc2cc(C#N)cnn12]
or a pharmaceutically acceptable salt thereof.

58. The compound of claim 54, wherein the compound is 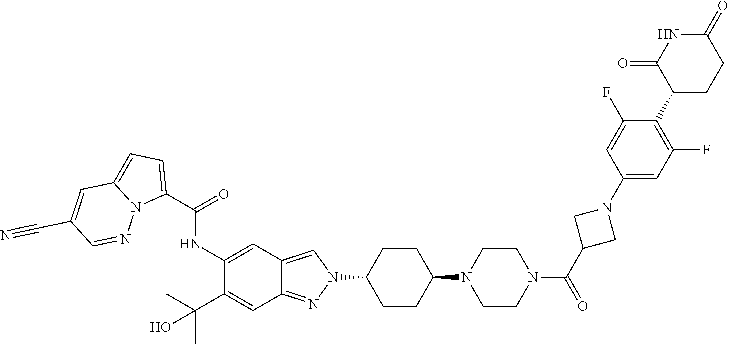 [SMILES: CC(C)(O)c1cc2nn([C@H]3CC[C@H](N4CCN(C(=O)C5CN(c6cc(F)c([C@H]7CCC(=O)NC7=O)c(F)c6)C5)CC4)CC3)cc2cc1NC(=O)c1ccc2cc(C#N)cnn12]
or a pharmaceutically acceptable salt thereof.

59. The compound of claim 54, wherein the compound is 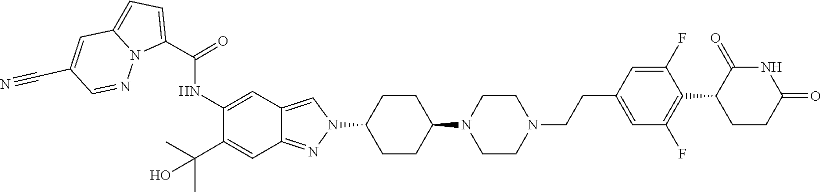 [SMILES: CC(C)(O)c1cc2nn([C@H]3CC[C@H](N4CCN(CCc5cc(F)c([C@H]6CCC(=O)NC6=O)c(F)c5)CC4)CC3)cc2cc1NC(=O)c1ccc2cc(C#N)cnn12]
or a pharmaceutically acceptable salt thereof.

60. The compound of claim 54, wherein the compound is 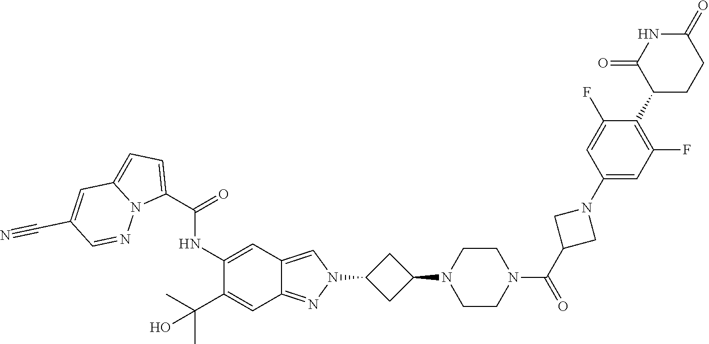 [SMILES: CC(C)(O)c1cc2nn([C@H]3C[C@H](N4CCN(C(=O)C5CN(c6cc(F)c([C@H]7CCC(=O)NC7=O)c(F)c6)C5)CC4)C3)cc2cc1NC(=O)c1ccc2cc(C#N)cnn12]
or a pharmaceutically acceptable salt thereof.

61. The compound of claim 54, wherein the compound is 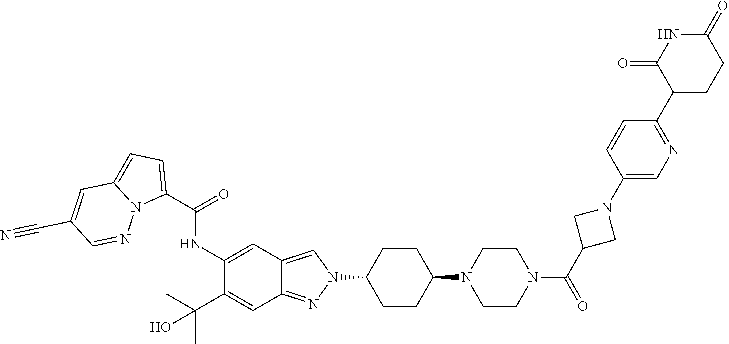 [SMILES: CC(C)(O)c1cc2nn([C@H]3CC[C@H](N4CCN(C(=O)C5CN(c6ccc(C7CCC(=O)NC7=O)nc6)C5)CC4)CC3)cc2cc1NC(=O)c1ccc2cc(C#N)cnn12]
or a pharmaceutically acceptable salt thereof.

62. The compound of claim 54, wherein the compound is 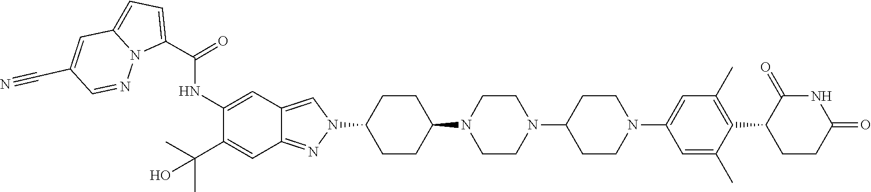 [SMILES: Cc1cc(N2CCC(N3CCN([C@H]4CC[C@H](n5cc6cc(NC(=O)c7ccc8cc(C#N)cnn78)c(C(C)(C)O)cc6n5)CC4)CC3)CC2)cc(C)c1[C@H]1CCC(=O)NC1=O]
or a pharmaceutically acceptable salt thereof.

63. The compound of claim 54, wherein the compound is 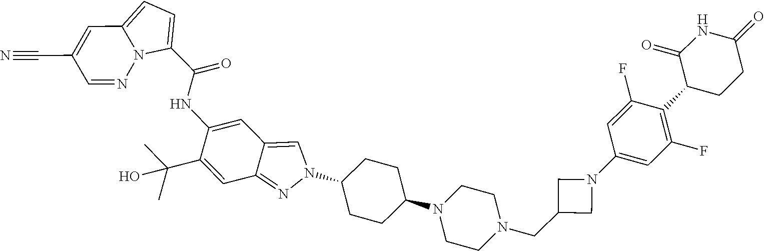 [SMILES: CC(C)(O)c1cc2nn([C@H]3CC[C@H](N4CCN(CC5CN(c6cc(F)c([C@H]7CCC(=O)NC7=O)c(F)c6)C5)CC4)CC3)cc2cc1NC(=O)c1ccc2cc(C#N)cnn12]
or a pharmaceutically acceptable salt thereof.

64. The compound of claim 54, wherein the compound is 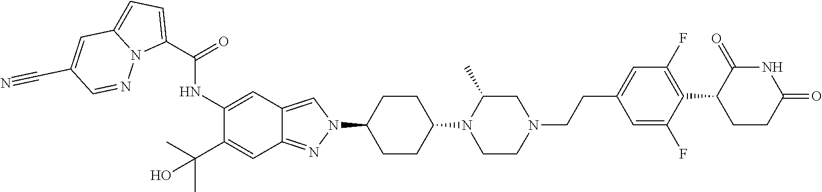 [SMILES: C[C@@H]1CN(CCc2cc(F)c([C@H]3CCC(=O)NC3=O)c(F)c2)CCN1[C@H]1CC[C@H](n2cc3cc(NC(=O)c4ccc5cc(C#N)cnn45)c(C(C)(C)O)cc3n2)CC1]
or a pharmaceutically acceptable salt thereof.

65. The compound of claim 54, wherein the compound is 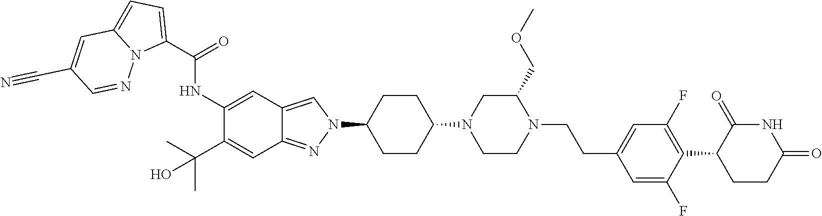 [SMILES: COC[C@H]1CN([C@H]2CC[C@H](n3cc4cc(NC(=O)c5ccc6cc(C#N)cnn56)c(C(C)(C)O)cc4n3)CC2)CCN1CCc1cc(F)c([C@H]2CCC(=O)NC2=O)c(F)c1]
or a pharmaceutically acceptable salt thereof.

66. The compound of claim 54, wherein the compound is 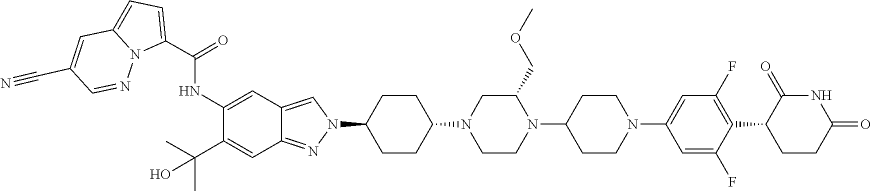 [SMILES: COC[C@H]1CN([C@H]2CC[C@H](n3cc4cc(NC(=O)c5ccc6cc(C#N)cnn56)c(C(C)(C)O)cc4n3)CC2)CCN1C1CCN(c2cc(F)c([C@H]3CCC(=O)NC3=O)c(F)c2)CC1]
or a pharmaceutically acceptable salt thereof.

67. The compound of claim 54, wherein the compound is 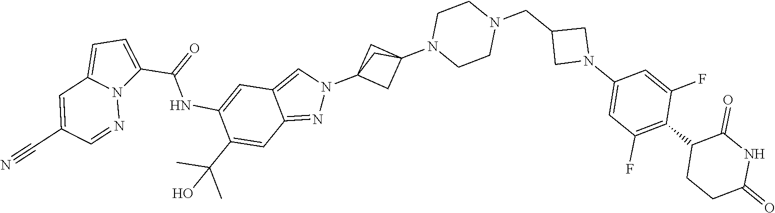
or a pharmaceutically acceptable salt thereof.

68. The compound of claim 54, wherein the compound is 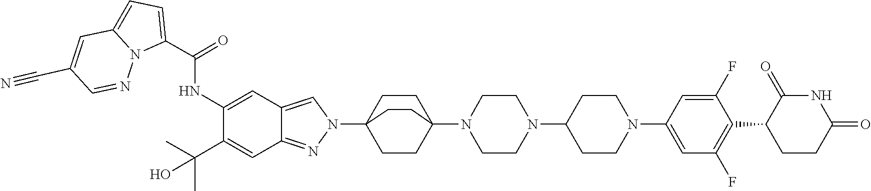 [SMILES: CC(C)(O)c1cc2nn(C34CCC(N5CCN(C6CCN(c7cc(F)c([C@H]8CCC(=O)NC8=O)c(F)c7)CC6)CC5)(CC3)CC4)cc2cc1NC(=O)c1ccc2cc(C#N)cnn12]
or a pharmaceutically acceptable salt thereof.

69. The compound of claim 54, wherein the compound is 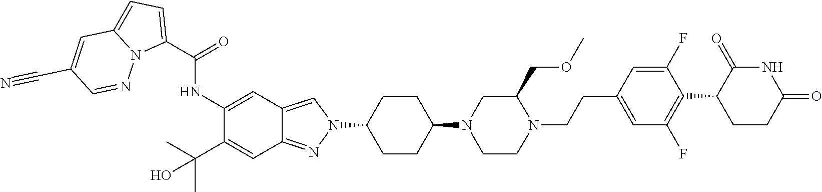 [SMILES: COC[C@@H]1CN([C@H]2CC[C@H](n3cc4cc(NC(=O)c5ccc6cc(C#N)cnn56)c(C(C)(C)O)cc4n3)CC2)CCN1CCc1cc(F)c([C@H]2CCC(=O)NC2=O)c(F)c1]
or a pharmaceutically acceptable salt thereof.

70. The compound of claim 54, wherein the compound is 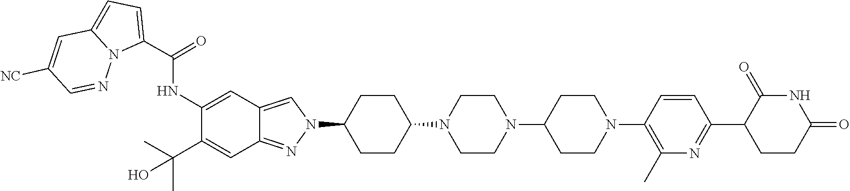 [SMILES: Cc1nc(C2CCC(=O)NC2=O)ccc1N1CCC(N2CCN([C@H]3CC[C@H](n4cc5cc(NC(=O)c6ccc7cc(C#N)cnn67)c(C(C)(C)O)cc5n4)CC3)CC2)CC1]
or a pharmaceutically acceptable salt thereof.

71. The compound of claim 54, wherein the compound is 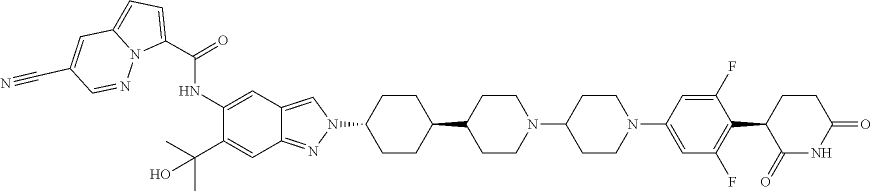 [SMILES: CC(C)(O)c1cc2nn([C@H]3CC[C@H](C4CCN(C5CCN(c6cc(F)c([C@H]7CCC(=O)NC7=O)c(F)c6)CC5)CC4)CC3)cc2cc1NC(=O)c1ccc2cc(C#N)cnn12]
or a pharmaceutically acceptable salt thereof.

72. The compound of claim 54, wherein the compound is 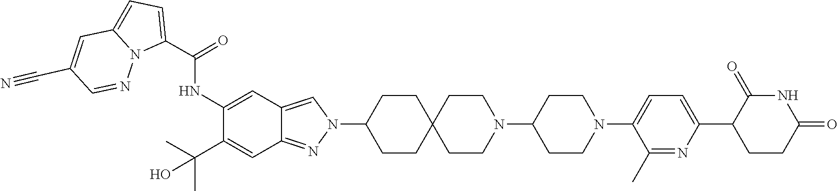 [SMILES: Cc1nc(C2CCC(=O)NC2=O)ccc1N1CCC(N2CCC3(CCC(n4cc5cc(NC(=O)c6ccc7cc(C#N)cnn67)c(C(C)(C)O)cc5n4)CC3)CC2)CC1]
or a pharmaceutically acceptable salt thereof.

73. A pharmaceutical composition comprising a compound or a pharmaceutically acceptable salt thereof of claim 54 and a pharmaceutically acceptable excipient.
